(19) United States
(12) Patent Application Publication
(10) Pub. No.: US 2021/0130833 A1
(43) Pub. Date: May 6, 2021

(54) BACTERIAL DEFENSE SYSTEMS AND METHODS OF IDENTIFYING THEREOF

(71) Applicants: THE BROAD INSTITUTE, INC., Cambridge, MA (US); MASSACHUSETTS INSTITUTE OF TECHNOLOGY, Cambridge, MA (US)

(72) Inventors: Feng Zhang, Cambridge, MA (US); Linyi Gao, Cambridge, MA (US)

(73) Assignees: THE BROAD INSTITUTE, INC., Cambridge, MA (US); MASSACHUSETTS INSTITUTE OF TECHNOLOGY, Cambridge, MA (US)

(21) Appl. No.: 17/085,937

(22) Filed: Oct. 30, 2020

Related U.S. Application Data
(60) Provisional application No. 62/928,269, filed on Oct. 30, 2019.
(60) Provisional application No. 63/051,161, filed on Jul. 13, 2020.

(51) Int. Cl. C12N 15/70; C12Q 1/689; C12N 9/14; C12N 9/78; C12N 9/12; C12N 9/22

(52) U.S. Cl. CPC C12N 15/70; C12Q 1/689; C12N 9/14; C12Y 306/01003; C12N 9/78; C12N 2800/80; C12N 9/1276; C12Y 207/07049; C12N 9/22; C07K 2319/00; C12Y 305/04004

(57) ABSTRACT

Engineered systems comprising components of defense systems identified in prokaryotes are provided.

### CROSS-REFERENCE TO RELATED APPLICATIONS

This application claims the benefit of U.S. Provisional Application No. 62/928,269, filed Oct. 30, 2019, and U.S. Provisional Application No. 63/051,161, filed Jul. 13, 2020. The entire contents of the above-identified applications are hereby fully incorporated herein by reference.

### STATEMENT REGARDING FEDERALLY SPONSORED RESEARCH

This invention was made with government support under Grant Nos. HG009761, MH110049, and HL141201 awarded by the National Institutes of Health. The government has certain rights in the invention.

### REFERENCE TO AN ELECTRONIC SEQUENCE LISTING

The contents of the electronic sequence listing (“BROD-4610US_ST25.txt”; Size is 2,039,992 bytes and it was created on Oct. 30, 2020) is herein incorporated by reference in its entirety.

### TECHNICAL FIELD

The subject matter disclosed herein is generally directed to bacterial defense systems and methods of identifying thereof.

### BACKGROUND

To survive from attacks by viruses (e.g., phages), bacteria have developed a variety of defense systems, including proteins and nucleic acids that help recognize and eliminate foreign proteins and nucleic acids, e.g., those from the infecting phages. A number of bacteria defense systems have been discovered, many of which have been adopted and engineered to tools in biotechnology. An example is the CRISPR-Cas systems, which recognize and cleave foreign RNA or DNA in bacteria and have been developed as a powerful gene editing tool. In view of the great potential of bacterial defense systems in biotechnology and new therapeutic or diagnostic applications, there is a need for identification of novel defense systems in a high throughput manner.

### SUMMARY

In one aspect, the present disclosure provides an engineered system comprising an ATPase and an adenosine deaminase. In some embodiments, the ATPase comprises a sequence of WP_012906049.1 or WP_155731552.1, and the adenosine deaminase comprises a sequence of WP_012906048.1 or WP_064360593.1. In some embodiments, the ATPase comprises 1100 or less amino acid residues. In some embodiments, the adenosine deaminase comprises 1100 or less amino acid residues. In some embodiments, the system further comprises a membrane protein. In some embodiments, the membrane protein comprises a SLATT domain or Csx27. In some embodiments, the system is configured to modify a target nucleic acid. In some embodiments, the target nucleic acid is RNA. In some embodiments, the modification of the target nucleic acid comprises causing an A to G mutation in the target nucleic acid. In some embodiments, the system further comprises one or more phage proteins. In some embodiments, the one or more phage proteins are in Tables 18A-18B.

In another aspect, the present disclosure provides an engineered system comprising one or more reverse transcriptases comprising one or more UG1, UG2, UG3, UG8, UG15, or UG16 reverse transcriptase. In some embodiments, the system comprises a first and a second reverse transcriptase. In some embodiments, the first and the second reverse transcriptases are comprised in a protein. In some embodiments, the system further comprises a SLATT domain. In some embodiments, the system further comprises a DNA polymerase. In some embodiments, the DNA polymerase is a family A DNA polymerase. In some embodiments, the system further comprises a serine protease domain linked to or associated with the reverse transcriptase. In some embodiments, the system further comprises an MBL domain. In some embodiments, the system further comprises a nitrilase. In some embodiments, the nitrilase and the one or more reverse transcriptases are comprised in a protein, and the nitrilase is at a C-terminus of the protein. In some embodiments, the system further comprises a non-coding RNA element. In some embodiments, the reverse transcriptase comprises an active site, e.g., (Y/F)×DD (SEQ ID NO: 1-2), where X is any amino acid.

In another aspect, the present disclosure provides an engineered system comprising a retron or one or more molecules encoded by the retron. In some embodiments, the retron is an Ec67 retron. In some embodiments, the retron is an Ec86 retron. In some embodiments, the retron is an Ec78 retron. In some embodiments, the retron is a Tol/interleukin 1 receptor (TIR) domain-associated retron. In some embodiments, the TIR domain has NAD+ hydrolase activity. In some embodiments, the retron is a topoisomerase-primase (TOPRIM) domain-associated retron. In some embodiments, the TOPRIM domain has nuclease activity.

In another aspect, the present disclosure provides an engineered system comprising an NTPase of a STAND (signal transduction ATPases with numerous associated domains) superfamily. In some embodiments, the system further comprises DUF4297, Mrr-like nuclease, SIR2, a trypsin-like serine protease, and/or a helical domain.

In another aspect, the present disclosure provides an engineered system comprising a von Willebrand factor (VWF), a PP2C-like serine/threonine protein phosphatase, and a serine/threonine kinase.

In another aspect, the present disclosure provides an engineered system comprising SIR2 or a function domain thereof.

In another aspect, the present disclosure provides an engineered system comprising a transmembrane ATPase.

In another aspect, the present disclosure provides an engineered system comprising an ATPase, QueC synthase, and TatD endonuclease.

In another aspect, the present disclosure provides an engineered system comprising a S8 peptidase.

In another aspect, the present disclosure provides an engineered system comprising DUF4011, a helicase, an a Vsr endonuclease.

In another aspect, the present disclosure provides an engineered system comprising a silent information regulator (SIR)2-DUF4020.

In another aspect, the present disclosure provides an engineered system comprising a Polymerase and Histidinol Phosphatase (PHP)-ATPase.

In another aspect, the present disclosure provides an engineered system comprising SIR2 and HerA.

In another aspect, the present disclosure provides an engineered system comprising DUF4297 and HerA.

In another aspect, the present disclosure provides an engineered system comprising DUF 1887.

In another aspect, the present disclosure provides an engineered system comprising DUF499, DUF3780, and DUF1156 methyltransferase and a helicase.

In another aspect, the present disclosure provides an engineered system comprising a type I-E CRISPR-associated ATPase.

In another aspect, the present disclosure provides an engineered system comprising ApeA.

In some embodiments, any one of the systems herein comprises two proteins fused together. In some embodiments, any one of the systems herein comprises one or more components in a retrotransposon system.

In another aspect, the present disclosure provides a polynucleotide comprising coding sequences for one or more proteins in the system herein.

In another aspect, the present disclosure provides a vector comprising a polynucleotide herein.

In another aspect, the present disclosure provides a cell comprising the polynucleotide herein.

In another aspect, the present disclosure provides a method of identifying a defense system in a microorganism, the method comprising: identifying genes of known defense systems in a plurality of genomes of the microorganism; recording candidate genes located within 10 kb or 10 open reading frames from the identified genes of known defense systems in the genomes; identifying homologs of each candidate gene in the genomes; and selecting candidate genes, wherein at least 10% of homologs of the candidate genes are within 5000 nucleotides or 5 genes from one or more known defense systems on the genomes.

In some embodiments, identifying genes of known defense systems comprises identifying known defense genes and filtering false positive hits among the identified known defense genes. In some embodiments, the method further comprises validating the selected candidate genes. In some embodiments, the homologs of the candidate genes share at least 70% sequence identity with the candidate genes and/or the homologs have an e-value of 10⁻⁵ or lower. In some embodiments, the recorded candidate genes are within 10 kb from the identified genes of known defense systems on the genomes. In some embodiments, at least 15% of homologs of the selected candidate genes are within 5000 nucleotides or 5 genes from one or more known defense systems on the genomes. In some embodiments, the plurality of genomes comprises at least 100,000 genomes. In some embodiments, the known defense systems comprise one or more of a CRISPR system, Type I RM and McrBC system, BREX-associated system, Zorya system, Wadjet system, Druantia-associated system, Hachiman system, Lamassu system, Thoeris-like system, Gabija system, Septu system, pAgo system, Shedu system, Kiwa system, DUF499-DUF1156 system, and Toxin/antitoxin system. In some embodiments, the microorganism is E. coli.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1Y. FIG. 1A shows diagrams of exemplary identified defense system comprising reverse transcriptase and nitrilase. FIG. 1B shows diagrams of exemplary identified defense system comprising a reverse transcriptase and a nitrilase, and a topoisomerase-primase (TOPRIM). FIG. 1C shows diagrams of exemplary identified defense system comprising a reverse transcriptase and TOPRIM. FIG. 1D shows diagrams of exemplary identified defense system comprising a reverse transcriptase. FIG. 1E shows diagrams of exemplary identified defense system comprising a deaminase. FIG. 1F shows diagrams of exemplary identified defense system comprising a transmembrane ATPase. FIG. 1G shows diagrams of exemplary identified defense system comprising an ATPase, QueC synthase, and TatD endonuclease. FIG. 1H shows diagrams of exemplary identified defense system comprising a protease. FIG. 1I shows diagrams of exemplary identified defense system comprising DUF4011 domain. FIG. 1J shows diagrams of exemplary identified defense system comprising an Hsp90 ATPase and SF2-family helicase. FIG. 1K shows diagrams of exemplary identified defense system comprising trypsin-STAND. FIG. 1L shows diagrams of exemplary identified defense system comprising DUF4297-STAND and another protein. FIG. 1M shows diagrams of another exemplary identified defense system comprising DUF4297-STAND. FIG. 1N shows diagrams of exemplary identified defense system comprising a STAND ATPase. FIG. 1O shows diagrams of another exemplary identified defense system comprising Mrr-STAND. FIG. 1P shows diagrams of exemplary identified defense system comprising VWA, phosphatase, and kinase. FIG. 1Q shows diagrams of exemplary identified defense system comprising SIR2 and a DUF4020 domain. FIG. 1R shows diagrams of exemplary identified defense system comprising SIR2. FIG. 1S shows diagrams of exemplary identified defense system comprising SIR2-STAND. FIG. 1T shows diagrams of exemplary identified defense system comprising PHP-ATPase. FIG. 1U shows diagrams of exemplary identified defense system comprising SIR2 and HerA. FIG. 1V shows diagrams of exemplary identified defense system comprising DUF1887. FIG. 1W shows diagrams of exemplary identified defense system comprising a CRISPR-associated enzyme and an ATPase. FIG. 1X shows diagrams of exemplary identified defense system comprising reverse transcriptase and a protease. FIG. 1Y shows figure legends used in FIGS. 1A-1X.

FIG. 2 shows diagrams of exemplary identified defense system comprising reverse transcriptase and amidase.

FIG. 3 shows diagrams of exemplary identified defense systems that comprise reverse transcriptase.

FIG. 4 shows an exemplary method of identifying defense systems.

FIG. 5 shows another exemplary method of identifying defense systems.

FIGS. 6A-6B show the examples of the identified bacterial defense systems, their domain structures, and their effects on phage growth.

FIG. 7 shows selected identified bacterial defense systems and mutated forms, and their effects on phage growth.

FIGS. 8A-8C: Domain-independent identification of novel systems that were enriched in defense islands. (FIG. 8A) Computational pipeline to identify uncharacterized putative defense systems across all sequenced bacterial and archaeal genomes. Defense systems were identified based on de novo analysis of amino acid sequences, independent of pre-existing protein domain annotations. Histograms of defense association probabilities for (FIG. 8B) selected known systems used as control and (FIG. 8C) novel seed genes (minimum 50 identified homologs). Seeds to the right of the dashed line (0.15) were selected for further analysis.

FIGS. 9A-9B: Experimental validation of 29 novel defense gene cassettes. (FIG. 9A) Experimental validation pipeline using phage plaque assays on E. coli heterologously expressing a cloned candidate defense system. (FIG. 9B) Anti-phage activity across a diverse panel of coliphages with dsDNA, ssDNA, and ssRNA genomes (mean of n=2 replicates). Also shown is a bar graph of the abundance of each system within sequenced bacterial and archaeal genomes. See also FIGS. 12-13.

FIGS. 10A-10E: RADAR employs a divergent adenosine deaminase that edits RNA in response to phage infection. (FIG. 10A) Examples of genomic loci containing three subtypes of RADAR (standalone, Csx27-associated, and SLATT-associated). (FIG. 10B) Mutations at putative rdrA and rdrB active sites abolish activity against phage T5. (FIG. 10C) Representative RNAseq reads from E. coli expressing either RADAR or an empty vector control. (FIG. 10D) Examples of editing sites in the host and phage RNA, with identified RNA secondary structures. (FIG. 10E) Growth kinetics of RADAR-containing E. coli in comparison with an empty vector control under varying multiplicity of infection (MOI).

FIGS. 11A-11C: A diversity reverse transcriptases (RTs) mediate antiviral immunity. (FIG. 11A) Examples of genomic loci containing novel antiviral RTs. Three validated RT systems are shown (with two representative subtypes for each system). Domain architectures and component essentiality of (FIG. 11B) non-retron RTs and (FIG. 11C) retron-like RTs. See also FIG. 15.

FIG. 12: Novel defense systems with diverse domain architectures. Graphics show domains identified using HHpred, with mutations at active sites.

FIG. 13: Representative plaques for phages T3, T7, φV-1, and φX174 (n=2 replicates) on E. coli strain C, corresponding to the right panel of FIG. 9B. A total of 5×10⁶ virions were deposited per spot, and images were acquired after 68 h incubation at 37° C.

FIG. 14: Abundance of defense systems within sequenced genomes stratified by phylum. Defense system homologs were predicted using a two-step HMM-based search across all sequenced bacterial and archaeal genomes in Genbank.

FIG. 15: Anti-phage defense activity for two RT-containing systems 28 and 29 (see also FIGS. 11A-11C). Ten-fold serial dilutions of phage were spotted on a soft agar overlay containing E. coli. D313 is the putative conserved active site aspartate for the family A DNA polymerase PolA.

FIGS. 16A-16C: Domain-independent prediction of putative antiviral defense systems. (FIG. 16A) Computational pipeline to identify uncharacterized putative defense systems across all sequenced bacterial and archaeal genomes. Defense systems were predicted based on analysis of amino acid sequences, independent of domain annotations. (FIG. 16B) Histograms of defense association frequencies before filtering and after neighborhood context-based filtering (minimum 50 homologs). Seeds to the right of the dashed line (0.1) were selected for further analysis. (FIG. 16C) Pie chart of the domain diversity among predicted defense genes, based on additional analysis using HHpred against pfam domains.

FIGS. 17A-17D: Candidate defense systems exhibit antiviral activity in a heterologous system. (FIG. 17A) Experimental validation pipeline using phage plaque assays on E. coli heterologously expressing a cloned candidate defense system. Example plaques (FIG. 17B) and zones of lysis (FIG. 17C) for six candidate defense systems. (FIG. 17D) Anti-phage activity across a panel of 12 coliphages with dsDNA, ssDNA, and ssRNA genomes (mean of n=2 replicates). The bar graph shows the abundance of each system within sequenced bacterial and archaeal genomes. Domains: MTase: methyltransferase; RT: reverse transcriptase; TIR: Toll/interleukin-1 receptor homology domain; TOPRIM: topoisomerase-primase domain; QueC: 7-cyano-7-deazaguanine synthase-like domain; SIR2: sirtuin; S/T phos: serine/threonine protein phosphatase; membrane: transmembrane helix; DUF: domain of unknown function. Proposed gene names (underlined): DRT: defense-associated reverse transcriptase; RADAR: phage restriction by ADAR; AVAST: antiviral ATPase/NTPase of the STAND superfamily; drs: defense-associated sirtuin; tmn: transmembrane NTPase; qat: QueC-like associated with ATPase and TatD DNAase; hhe: HEPN, helicase, and Vsr endonuclease; mza: MutL, Z1, and AIPR; upx: uncharacterized (P)D-(D/E)-XK defense protein; ppl: polymerase/histidinol phosphatase-like.

FIGS. 18A-18F: RADAR mediates RNA editing in response to phage infection. (FIG. 18A) Examples of genomic loci containing three subtypes of RADAR (standalone, Csx27-associated, and SLATT-associated). (FIG. 18B) Essentiality of the core RADAR genes rdrAB and the accessory gene rdrD against phages T2 and T5. (FIG. 18C) Representative RNAseq reads from E. coli expressing either RADAR or an empty vector control. (FIG. 18D) Expression of phage T2 RNA relative to total host RNA in E. coli containing RADAR. Each dot represents a phage gene. Cells were infected at a multiplicity of infection (MOI) of 2. The p value was determined by a Wilcoxon signed-rank test. (FIG. 18E) Representative editing sites in the host and phage transcriptomes, with corresponding predicted RNA secondary structures. (FIG. 18F) Growth kinetics of RADAR-containing E. coli in comparison with an empty vector control under varying MOI by phage T2.

FIGS. 19A-19E: Diverse families of reverse transcriptases (RTs) mediate antiviral defense. (FIG. 19A) Examples of genomic loci containing two validated RT systems (DRT type 1 and type 3), with two representative subtypes shown for each system. (FIG. 19B) Essential components of non-retron RTs (left panel) and retrons (right panel). (FIG. 19C) Effect of defense RTs on the expression of phage T2 genes in E. coli infected at an MOI of 2. (FIG. 19D) RNAseq reads mapping to the DRT type 3 system. (FIG. 19E) Predicted secondary structure of the highly expressed non-coding RNA identified in (FIG. 19D).

FIG. 20: Domain architectures and mutational analysis of additional defense systems. Graphics show domains identified using HHpred, and stars indicate locations of active site mutations. Bar graphs (n=4 replicates per bar) show either log₁₀ fold change of efficiency of plating (for phages T2, P1, and λ) or log₂ fold change in the area of the zone of lysis (for phages T7 and φV-1) relative to the empty vector control. MBL: metallo β-lactamase; SIR2: sirtuin; HerA: helicase; QueC: 7-cyano-7-deazaguanine synthase-like domain; TatD: DNAse; vWA: von Willebrand factor type A; PHP: polymerase/histidinol phosphatase; MTase: methyltransferase; PLD: phospholipase D.

FIGS. 21A-21C: Selection of filtering thresholds for prediction of putative defense genes. Contour density plots for predicted (FIG. 21A) toxin-antitoxin/abi genes, (FIG. 21B) mobilome genes, and (FIG. 21C) CRISPR-Cas genes. Boxes indicated the parameter thresholds selected for filtering putative defense genes.

FIG. 22: Summary of tested homologs of candidate defense systems, stratified by source organism (Enterobacteriaceae vs. non-Enterobacteriaceae). Systems 1-29 correspond to the numbering in FIG. 17D.

FIG. 23: Representative zones of lysis for phages T3, T7, V-1, and X174 on E. coli strain C (n=2 replicates each), corresponding to the right panel of FIG. 2D. A total of 5×106 virions were deposited per spot.

FIG. 24: Abundance of validated defense systems within sequenced genomes, stratified by phylum. Defense system homologs were predicted using a two-step HMM-based search across all bacterial and archaeal genomes in Genbank (see Methods).

FIGS. 25A-25B: Domain and locus architecture of the RADAR deaminase. (FIG. 25A) Unrooted neighbor-joining tree of RdrB homologs with the Jukes-Cantor genetic distance model. Distinct clades of RADAR incorporate accessory membrane proteins RdrC (Csx27) or RdrD (SLATT). (FIG. 25B) RdrB contains a split deaminase domain (red) with uncharacterized insertions. Domain boundaries were predicted using HHpred. Percent identity was calculated from a multiple sequence alignment of 535 representative homologs with at most 98% pairwise similarity.

FIGS. 26A-26B: Deamination by the RADAR system occurs only on adenosines within RNA and requires both RADAR genes. (FIG. 26A) Empirical probability mass functions of editing frequency for each of the 12 possible RNA base changes, calculated using the highest-expressed mRNAs in the transcriptome of E. coli K-12 (ATCC25404) expressing the RADAR system from Citrobacter rodentium DBS100. Cells were harvested 1 hr after infection by phage T2 at an MOI of 2. (FIG. 26B) Editing frequency at a selected site within the transfer messenger RNA (tmRNA) locus (RNA or DNA). Sequences below the graphs show representative reads.

FIG. 27: RADAR preferentially deaminates adenosines within loop regions of RNA stem-loops. Predicted RNA secondary structures of the 48 highest-expressed strong RADAR editing sites (50% editing).

FIGS. 28A-28F: Effect of expression of specific phage genes on RNA editing by RADAR. (FIG. 28A) Phage genes were cloned after IPTG-inducible T7 promoter and transformed into E. coli heterologously expressing the RADAR system from Citrobacter rodentium DBS100. (FIG. 28B) Structure of E. coli transfer messenger RNA (tmRNA) (PDBID: 6Q9A), highlighting adenosines strongly edited by RADAR. (FIG. 28C) Scatter plots of RNA editing frequencies for two replicates. Each dot represents a different phage fragment. (FIG. 28D) Locations of fragments on the phage T2 genome. Each colored box represents a distinct fragment. (FIG. 28E) RNA editing frequencies of the fragments shown in (FIG. 28D) at A93 and A121 of the E. coli tmRNA. (FIG. 28F) RNA editing frequencies induced by expression of RADAR with individual genes within six of the highest-activity fragments identified in (FIG. 28D). Purple squares indicate active site mutants created by site-directed mutagenesis. dam=DNA adenine methyltransferase; a-gt: DNA alpha glucosyltransferase; gp50: head completion protein; gp2: DNA end protector protein; frd: dihydrofolate reductase; rnh: RNase H; dsbA: dsDNA binding protein; denA: endonuclease II.

FIGS. 29A-29C: Mutational analysis of three RT-containing defense systems. Active site mutations abolish defense activity against phage T5 for the (FIG. 29A) RT (UG2), (FIG. 29B) RT (UG15), and (FIG. 29C) retron+ATPase+HNH (Ec78) systems. The ATPase and HNH proteins in Ec78 comprise the Septu defense system.

FIGS. 30A-30C: The nitrilase domain of the RT (UG1) defense system forms a distinct Glade among nitrilase enzymes. (FIG. 30A) Stacked histogram of E-values of sequence-profile matches (RPSBLAST) between prokaryotic proteins in Genbank against a custom position-specific scoring matrix for the RT (UG1) nitrilase domain (minimum 20% coverage). Proteins matching a known nitrilase PSSM from the CDD database (E-value −10⁻⁶; minimum 40% coverage) are shown in green. (FIG. 30B) Unrooted neighbor-joining tree of the reverse transcriptase (RT) domain in nitrilase-associated RTs (n=588). Colors indicate distinct clades (cutoff tree distance 0.15). (FIG. 30C) Unrooted neighbor-joining tree of the nitrilase domain in proteins in (FIG. 30B) with the same color scheme (based on RT domain Glade). Also included in the tree are the non-RT-associated nitrilases (green) that are most similar to the nitrilase domain in RT (UG1) among all prokaryotic proteins.

FIG. 31: Effect of mutations in the multi-copy single-stranded DNA (msDNA) hairpin on defense activity for the Ec86 retron from E. coli BL21.

FIGS. 32A-32B: Bacterial densities over time for (FIG. 32A) retron-TIR, RT-nitrilase (UG1), and RT (UG3)+RT (UG8) defense systems infected with phage T2 and (FIG. 32B) additional defense systems infected with phage T7.

FIGS. 33A-33C: Phage and prophage association frequencies for validated defense system clusters. (FIG. 33A) Overall association frequency for 28 defense systems in this study. The rexA immunity gene from phage lambda is shown in red. (FIG. 33B) Per-system analysis of the distribution of phage association frequencies for each associated cluster in (FIG. 33A). (FIG. 33C) Example of the transmembrane ATPase located within an incomplete prophage.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

As used herein, when an enzyme is mentioned, the term also includes a functional domain of the enzyme. For example, a reverse transcriptase may refer to a reverse transcriptase protein or a reverse transcriptase domain.

A protein or nucleic acid derived from a species means that the protein or nucleic acid has a sequence identical to an endogenous protein or nucleic acid or a portion thereof in the species. The protein or nucleic acid derived from the species may be directly obtained from an organism of the species (e.g., by isolation), or may be produced, e.g., by recombination production or chemical synthesis.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides various types of bacterial defense systems and the methods of identifying thereof. In some aspects, the present disclosure includes a number of newly identified defense systems. In some embodiments, the systems may be engineered, e.g., to have a desired activity or function. The engineered systems may be used as tools (e.g., to manipulate expression and/or activity of target genes or proteins) in biotechnology and medical applications. In one example, the system comprises an ATPase and an adenosine deaminase. Such system may be engineered to function as a base editor for gene editing applications. In another example, the system comprises one or more reverse transcriptases. In another example, the system comprises a retron or one or more molecules encoded by the retron. In another example, the system comprises an NTPase of a STAND (signal transduction ATPases with numerous associated domains) superfamily.

In another aspect, the present disclosure includes methods of identifying novel defense systems. In general, the methods are based on the fact that defense systems are often clustered in bacterial genomes. In some embodiments, the methods comprise identifying genes of known defense systems in a plurality of genomes of a bacterial species, identifying homolog genes close (e.g., within 10 kb) of the known defense systems, and selecting candidate genes among these homologs. For example, candidate genes may be selected when at least 10% of homologs of the genes are within 5000 nucleotides or 5 genes from one or more defense systems.

### Defense Systems

In one aspect, the present disclosure provides defense systems in prokaryotes such as bacteria. The defense systems may include proteins and nucleic acids that play roles in the defense of virus and other foreign organisms' attack and invasion. The present disclosure also includes nucleic acids encoding the components of the defense systems and vectors comprising such nucleic acids. The functions and applications of the defense systems herein are not limited to defending bacteria from foreign organisms (e.g., virus). Rather the defense systems may be used in various applications, e.g., as research tools and reagents, therapeutic agents, and diagnostic agents. In some cases, a defense system may be engineered to have a desired function. Such engineered defense system may not have a function related to defending bacteria from foreign organisms.

The defense systems provided herein may be of various types. These defense systems may comprise one or more enzymes that can manipulate (e.g., cleave, eliminate, degrade, etc.) the proteins and nucleic acids from the foreign organisms. In some examples, a host cell with the defense system may be resistant to foreign organism attacks. The term “resistance” to, for example, foreign nucleic acid invasion, encompasses a decrease in activity (e.g. phage genomic replication, phage lysogeny, circularization of phage genome) in bacteria expressing a functional defense system in comparison to bacteria of the same species under the same developmental stage (e.g. culture state) which does not express a functional defense system. According to specific embodiments the decrease provided by such resistance to foreign organism invasion is at least 1.5-fold, at least 2-fold, at least 3-fold, at least 5-fold, at least 10-fold, or at least 20-fold as compared to same in the absence of the functional defense system.

In some embodiments, the defense systems have an anti-phage activity. The term “anti-phage activity” or “resistant to infection by at least one phage” may encompasses an activity providing increased resistance of a host cell to infection by at least one phage in comparison to the host cell of the same species under the same developmental stage (e.g. culture state) which does not express the functional defense system. In some embodiments, a host cell may comprise a microbial cell. In some embodiments, a host comprises a bacterium. Anti-phage activity or resistance of a host cell to infection by at least one phage may be determined by, for example but not limited to, bacterial viability, phage lysogeny, phage genomic replication or phage genomic degradation, or a combination thereof.

In some embodiments, the defense systems may provide a host cell with resistance to foreign nucleic acid invasion. In some embodiments, a defense system described herein, provides the host cell with resistance to a foreign nucleic acid invasion, wherein the foreign nucleic acid invasion comprises resistance to at least one phage infection, or resistance to plasmid transformation, or a combination of resistance to at least one phage infection and resistance to plasmid transformation. In some embodiments, it is the combination of defense systems that provides a host cell with resistance to a foreign nucleic acid invasion. One skilled in the art would appreciate that defense against a foreign nucleic acid invasion may encompass, defending against entry of a foreign nucleic acid into the host cell, as well as, defending against the actions of a foreign nucleic acid that has entered the host cell. In some embodiments, defense against a foreign nucleic acid invasion comprises defense from phage infection. In some embodiments, defense against a foreign nucleic acid invasion comprises defense from plasmid transformation. In some embodiments, defense against a foreign nucleic acid invasion comprises defense against entry of a conjugative element. In some embodiments, defense against a foreign nucleic acid invasion comprises defense against any combination of phage infection, plasmid transformation, and entry of a conjugative element.

In some embodiments, the components in the system may be heterologous, i.e., they do not naturally occur together in the same cell or an organism.

The components in a system herein may be derived from the same or different prokaryotes. In some cases, the components may be engineered to be optimized for expressing in eukaryotic (e.g., mammalian) cells.

### Gene Clusters

In some embodiments, the components of a defense system may be in a gene cluster in a prokaryotic cell. The terms “gene cluster”, “cassette of genes”, “cassette”, and “components of a system”, may in some embodiments herein be used interchangeably having all the same meanings and qualities. In some embodiments, each gene of a “cassette of genes” comprises a nucleic acid sequence encoding a polypeptide component of the defense system. In some embodiments, a “cassette of genes” comprises nucleic acid sequences encoding components of the defense system including open reading frames encoding defense system polypeptide components, regulatory sequences, and non-coding RNAs. A skilled artisan would appreciate that a “cassette of genes” may encompass an operon. In some embodiments, a cassette of genes comprises regulatory sequences. In some embodiments, a cassette of gene comprises non-coding RNAs.

### Host Cells

The defense systems may be from or originate from microorganisms such as bacteria or archaea. In some embodiments, the defense may be from or originate from bacteria. As used herein, when a defense system originates form a species, it may be the wild type defense system in the species, or a homolog of the wild type defense system in the species. The defense system that is a homolog of the wild type defense system in the species may comprise one or more variations (e.g., mutations, truncations, etc.) of the wild type defense system. The terms “ortholog” and “homolog” are well known in the art. By means of further guidance, a “homolog” of a protein as used herein is a protein of the same species which performs the same or a similar function as the protein it is a homolog of. Homologous proteins may but need not be structurally related, or are only partially structurally related. An “ortholog” of a protein as used herein is a protein of a different species which performs the same or a similar function as the protein it is an ortholog of. Orthologous proteins may but need not be structurally related, or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225.). See also Shmakov et al. (2015) for application in the field of CRISPR-Cas loci. Homologous proteins may but need not be structurally related, or are only partially structurally related.

In some example, the host cells are E coli. In some embodiments, the bacteria may be gram positive bacteria. The term “Gram-positive bacteria” as used herein refers to bacteria characterized by having as part of their cell wall structure peptidoglycan as well as polysaccharides and/or teichoic acids and are characterized by their blue-violet color reaction in the Gram-staining procedure. Representative Gram-positive bacteria include: Actinomyces spp., Bacillus anthracis, Bifidobacterium spp., Clostridium botulinum, Clostridium perfringens, Clostridium spp., Clostridium tetani, Corynebacterium diphtherias, Corynebacterium jeikeium, Enterococcus faecalis, Enterococcus faecium, Erysipelothrix rhusiopathiae, Eubacterium spp., Gardnerella vaginalis, Gemella morbillorum, Leuconostoc spp., Mycobacterium abcessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Proprionibacterium spp., Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), Streptococcus salivarius, and Streptococcus sanguis.

In some embodiments, the term “Gram-negative bacteria” as used herein refer to bacteria characterized by the presence of a double membrane surrounding each bacterial cell. Representative Gram-negative bacteria include Acinetobacter calcoaceticus, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chalmydia pneumoniae, Chlamydia psittaci, Chlamydia trachomatis, to Chromobacterium violaceum, Citrobacter spp., Eikenella corrodens, Enterobacter aerogenes, Escherichia coli, Flavobacterium meningosepticum, Fusobacterium spp., Haemophilus influenzae, Haemophilus spp., Helicobacter pylori, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhi, Serratia marcescens, Shigella spp., Treponema carateum, Treponema pallidum, Treponema pallidum endemicum, Treponema pertenue, Veillonella spp., Vibrio cholerae, Vibrio vulnificus, Yersinia enterocolitica, and Yersinia pestis.

### Examples of Systems

A system provided herein may include one or more enzymes or functional protein domains, and/or polynucleotides encoding thereof. The systems may comprise one or more wild type proteins and/or polynucleotides. In certain cases, the systems may be engineered systems, e.g., comprising one or more mutations or variants compared to corresponding wild type counterparts.

In some embodiments, the systems herein may be configured to modify a nucleic acid, e.g., DNA, RNA, or a hybrid or duplex of RNA and DNA. In one example, the systems may be configured to modify RNA.

The systems and components thereof may be or share sequence homology (e.g., sequence identity) with the example systems and components herein. In some embodiments, the systems or components thereof may share at least 50%, at least 60%, at least 70%, at least 80%, at least 90%, at least 95%, at least 99%, or 100% sequence homology (e.g., sequence identity) with the example systems or components herein.

### Systems Comprising ATPase and Adenosine Deaminase

In some examples, the systems comprise an ATPase and an adenosine deaminase. The ATPase may be a KAP-family ATPase. In some cases, the ATPase may comprise 1500 or less, e.g., 1400 or less, 1300 or less, 1200 or less, 1100 or less, 1000 or less, 950 or less, 900 or less, 850 or less, 800 or less, 750 or less, 700 or less, 650 or less, 600 or less, 500 or less, 400 or less, 300 or less, 200 or less, 100 or less amino acid residues. In one example, the ATPase may comprise 1000 or less amino acid residues. In certain examples, the ATPase may comprise 900 or less amino acid residues. In some cases, the adenosine deaminase may comprise 1500 or less, e.g., 1400 or less, 1300 or less, 1200 or less, 1100 or less, 1000 or less, 950 or less, 900 or less, 850 or less, 800 or less, 750 or less, 700 or less, 650 or less, 600 or less, 500 or less, 400 or less, 300 or less, 200 or less, 100 or less amino acid residues. In one example, the adenosine deaminase may comprise 1000 or less amino acid residues. In certain examples, the adenosine deaminase may comprise 900 or less amino acid residues.

In some examples, the system comprises an ATPase that is or share at least 50%, at least 60%, at least 70%, at least 80%, at least 90%, at least 95%, at least 99%, or 100% sequence homology (e.g., sequence identity) with the sequence of WP_012906049.1 and a adenosine deaminase that is or share at least 50%, at least 60%, at least 70%, at least 80%, at least 90%, at least 95%, at least 99%, or 100% sequence homology (e.g., sequence identity) with the sequence of WP_012906048.1. In some examples, the system comprises an ATPase that is or share at least 50%, at least 60%, at least 70%, at least 80%, at least 90%, at least 95%, at least 99%, or 100% sequence homology (e.g., sequence identity) with the sequence of WP_155731552.1 and a adenosine deaminase that is or share at least 50%, at least 60%, at least 70%, at least 80%, at least 90%, at least 95%, at least 99%, or 100% sequence homology (e.g., sequence identity) with the sequence of WP_064360593.1.

In some embodiments, the system comprising ATPase and an adenosine deaminase may further comprise one or more proteins or polypeptide domains. In some examples, the system may further comprise a membrane protein or domain. In certain examples, the system further comprises a SMODS and LOG-Smf/DprA-Associating Two TM (SLATT) domain. In certain examples, the system further comprises a CRISPR ancillary protein. The type VI-B CRISPR ancillary protein, e.g., Csx27.

In some embodiments, the systems may be engineered to function as a base editor in gene editing applications. For example, the systems may modify a nucleic acid. The modification may cause an A to G mutation in a nucleic acid. In some cases, the systems may modify RNA. In some cases, the systems may modify DNA.

In some embodiments, the adenosine deaminase may be those described in International Patent Publication Nos. WO2019071048, WO2019084063, WO2019126716, WO2019126709, WO2019126762, and WO2019126774; Cox DBT, et al., RNA editing with CRISPR-Cas13, Science. 2017 Nov. 24; 358(6366):1019-1027; Abudayyeh 00, et al., A cytosine deaminase for programmable single-base RNA editing, Science 26 Jul. 2019: Vol. 365, Issue 6451, pp. 382-386; Gaudelli N M et al., Programmable base editing of A⋅T to G⋅C in genomic DNA without DNA cleavage, Nature volume 551, pages 464-471 (23 Nov. 2017); Komor A C, et al., Programmable editing of a target base in genomic DNA without double-stranded DNA cleavage. Nature. 2016 May 19; 533(7603):420-4, or any variants, homologs, or orthologs thereof.

In some embodiments, the system further comprise one or more phage proteins. Examples of phage proteins include those in Tables 18A-18B.

### Systems Comprising Reverse Transcriptase(s)

In some examples, the systems herein comprise one or more reverse transcriptases. A reverse transcriptase refers to an enzyme capable of synthesizing DNA strand (e.g., complementary DNA or cDNA) using RNA as a template. In some embodiments, the reverse transcriptase is error prone. For example, the reverse transcriptase may have low proof-reading ability. For example, the reverse transcriptase may introduce one or more errors (i.e., nucleotides that are not complementary to the corresponding nucleotides on the template). Examples of reverse transcriptases include the transcriptases from Vibrio harveyi ML phage, Bifidobacterium longum, Bacteroides thetaiotaonicron, Treponema denticola, cyanobacteria, such as Trichodesmium erythrism, the genus Nostoc, or Nostoc punctiforme.

As used herein, the reverse transcriptase may be full-length reverse transcriptase or a functional fragment thereof. A functional fragment of a full-length reverse transcriptase may be a polypeptide that is shorter than the full-length reverse transcriptase but has reverse transcriptase activity. For example, a functional fragment of a full-length reverse transcriptase may have at least about 50%, at least about 60%, at least about 70, % at least about 80%, at least about 90%, at least about 95%, at least about 99%, or at least about 100% of the activity of the corresponding reverse transcriptase. The reverse transcriptase activity may be measured as amount of cDNA generated with certain amount of RNA template.

For example, the systems may comprise a first reverse transcriptase and a second reverse transcriptase. The first and the second reverse transcriptases may be comprised in the same protein. The first and the second reverse transcriptase may be the same. In certain cases, the first and the second reverse transcriptase may be the different. The reverse transcriptase may be error prone.

Examples of reverse transcriptases include UG1, UG2, UG3, UG8, UG15, or UG16 reverse transcriptases. In some examples, the system comprises an UG1 reverse transcriptase that is or share at least 50%, at least 60%, at least 70%, at least 80%, at least 90%, at least 95%, at least 99%, or 100% sequence homology (e.g., sequence identity) with the sequence of WP_115196278.1. In some examples, the system comprises an U2 reverse transcriptase that is or share at least 50%, at least 60%, at least 70%, at least 80%, at least 90%, at least 95%, at least 99%, or 100% sequence homology (e.g., sequence identity) with the sequence of WP_012737279.1. In some examples, the system comprises an UG3 reverse transcriptase that is or share at least 50%, at least 60%, at least 70%, at least 80%, at least 90%, at least 95%, at least 99%, or 100% sequence homology (e.g., sequence identity) with the sequence of 087902017.1 and an U8 reverse transcriptase that is or share at least 50%, at least 60%, at least 70%, at least 80%, at least 90%, at least 95%, at least 99%, or 100% sequence homology (e.g., sequence identity) with the sequence of WP_062891751.1. In some examples, the system comprises an UG15 reverse transcriptase that is or share at least 50%, at least 60%, at least 70%, at least 80%, at least 90%, at least 95%, at least 99%, or 100% sequence homology (e.g., sequence identity) with the sequence of GCK53192.1. In some examples, the system comprises an UG16 reverse transcriptase that is or share at least 50%, at least 60%, at least 70%, at least 80%, at least 90%, at least 95%, at least 99%, or 100% sequence homology (e.g., sequence identity) with the sequence of WP_001524904.1.

In some examples, the systems comprising one or more reverse transcriptases may further comprise one or more proteins or polypeptide domains. In some examples, the systems further comprise a Cas protein, e.g., Cas1. In some examples, the systems further comprise Abi. In some examples, the systems further comprise a nitrilase-family C—N hydrolase. In some examples, the systems further comprise a DNA polymerase. The DNA polymerase may be a family A DNA polymerase. In some examples, the systems further comprise a nitrilase. In some examples, the systems comprise a protein comprising one or more reverse transcriptases and a nitrilase domain. The nitrilase domain may be at the C-terminus of the protein. In some examples, the systems further comprise a topoisomerase-primase (TOPRIM), and a nitrilase. In some examples, the systems further comprise a Tol/interleukin 1 receptor (TIR). In some examples, the systems further comprise a protease. The systems may further comprise a serine protease domain linked to or associated with the reverse transcriptase. In some examples, the systems further comprise an integrase. In some examples, the systems further comprise a transposase. In some examples, the systems further comprise an MBL domain.

In some cases, the system may comprise a polynucleotide encoding the reverse transcriptase. In certain examples, the polynucleotide comprising the variable region and/or the template region may comprise a coding sequence for the reverse transcriptase. In some examples, the polynucleotide encoding the reverse transcriptase may be different from the polynucleotide comprising the variable region and/or the template region.

In some embodiments, the reverse transcriptase comprises an active site, e.g., (Y/F)×DD (SEQ ID NOs: 1-2), where X is any amino acid.

### Systems Comprising Retrons or Molecules Encoded by Retrons

In some examples, the systems herein comprise one or more retrons or molecules encoded by retrons. As used herein, a retron refers to a genetic element (e.g., a DNA molecule) which encodes components enabling the synthesis of branched RNA-linked single stranded DNA (msDNA) and a reverse transcriptase. Molecules encoded by retrons includes retron msr RNA that is the non-coding RNA produced by retron elements and is the immediate precursor to the synthesis of msDNA. Molecules encoded by retrons also include the reverse transcriptase and the corresponding RNA (e.g., mRNA).

In some examples, the retron is Ec67 retron. In some examples, the retron is Ec86 retron. In some examples, the retron is Ec78 retron. In some examples, the retron is TIR domain-associated retron. The TIR domain may have NAD+ hydrolase activity. In some examples, the retron is TOPRIM domain-associated retron. The TOPRIM domain may have nuclease activity.

### Systems Comprising STAND NTPase

In some examples, the systems herein comprise one or more NTPases of a STAND (signal transduction ATPases with numerous associated domains) superfamily. In some examples, the systems comprising the NTPase may further comprise one or more proteins or polypeptide domains, such as DUF4297, Mrr-like nuclease, SIR2, a trypsin-like serine protease, and/or a helical domain.

### Additional Examples of Systems

In some examples, the system may comprise a von Willebrand factor (VWF), a PP2C-like serine/threonine protein phosphatase, and a serine/threonine kinase. In some examples, the system may comprise SIR2 or a function domain thereof.

In some examples, the system may comprise a reverse transcriptase and a nitrilase. In some examples, the system may comprise a reverse transcriptase and a nitrilase, and a topoisomerase-primase (TOPRIM). In some examples, the system may comprise a reverse transcriptase and TIR. In some examples, the system may comprise an Ec67 retron. In some examples, the system may comprise Ec86 retron. In some examples, the system may comprise a reverse transcriptase. In some examples, the system may comprise two reverse transcriptases. In some examples, the system may comprise adenosine deaminase. In some examples, the system may comprise KAP ATPase. In some examples, the system may comprise KAP TatD. In some examples, the system may comprise a transmembrane ATPase. In some examples, the system may comprise an ATPase, QueC synthase, and TatD endonuclease. In some examples, the system may comprise S8 peptidase. In some examples, the system may comprise a DFU4011 domain. In some examples, the system may comprise a DFU4011 domain, a helicase, and a Vsr endonuclease. In some examples, the system may comprise a DUF3684 Hsp90-like ATPase and a helicase. In some examples, the system may comprise Trypsin-AAA35. In some examples, the system may comprise DUF4297-AAA3 and another protein. In some examples, the system may comprise DUF4297-AAA35. In some examples, the system may comprise AAA35. In some examples, the system may comprise RE-AAA35. In some examples, the system may comprise VWA and phosphatase and a kinase. In some examples, the system may comprise SIR2-DUF4020. In some examples, the system may comprise SIR2-STAND-TPR. In some examples, the system may comprise Polymerase and Histidinol Phosphatase (PHP)-ATPase. In some examples, the system may comprise PHP-SMC. In some examples, the system may comprise SIR2 and HerA. In some examples, the system may comprise DUF4297 and HerA. In some examples, the system may comprise Unknown-DUF1887. In some examples, the system may comprise DUF262 and DUF262-HNH. In some examples, the system may comprise DUF499, DUF3780, DUF1156 methyltransferase, and helicase. In some examples, the system may comprise Type I-E CRISPR-associated protein. In some examples, the system may comprise RT-protease. In some examples, the system may comprise ApeA.

Details of these systems are shown in Tables 1, 2, 5, 6, 9, 10, 12, 13, 15A, and 16A. Sequences of example systems are shown in Tables 6, 12, 15A, 15B, 15C, 16A, and 16B.

TABLE 1

| | # genes in | | | | |
|---|---|---|---|---|---|
| Construct | operon | Short Description | Donor Strain | Diagram File Name | Note |
| pLG018 | 1 | RT-nitrilase | Klebsiella pneumoniae | pLG018_RT-nitrilase | UG1/UG6 in Zimmerly & |
| | | | NCTC9143 | | Wang (2015) |
| pLG022 | 1 | TOPRIM-RT-nitrilase | Vogesella indigofera | pLG022_TOPRIM- | UG10 in Zimmerly & Wang |
| | | | DSM3303 | RT-nitrilase | (2015) |
| pLG024 | 1 | RT-TIR | Shigella dysenteriae | — | Novel retron |
| | | | NCTC2966 | | |
| pLG026 | 1 | Ec67 retron | Escherichia coli | pLG026_RT-TOPRIM | Ec67 retron (reported in |
| | | | NCTC8623 | (retron | Lampson et al. Science 1989; |
| | | | | | function unknown until |
| | | | | | present study) |
| pLG199 | 1 | Ec86 retron | Escherichia coli BL21 | — | Ec86 retron (reported in Lim |
| | | | | | et al. Cell 1989; function |
| | | | | | unknown until present study) |
| pLG028 | 1 | RT | Escherichia coli | pLG028_RT | |
| | | | 21-C8-A | | |
| pLG125 | 2 | RT-x2 | Escherichia coli | — | Two RTs acting in concert; |
| | | | ECOR12 | | UG3/UG8 in Zimmerly & |
| | | | | | Wang (2015) |
| pLG032 | 2 | Adenosine deaminase | Citrobacter rodentium | pLG032_Deaminase | ATPase + highly divergent |
| | | | DBS100 | | adenosine deaminase |
| pLG034 | 1 | KAP ATPase | Escherichia coli | pLG034_KAP- | Large transmembrane |
| | | | ECOR25 | transmembrane | ATPase; described |
| | | | | | computationally in Aravind et |
| | | | | | al. Genome Biol (2004) |
| pLG037 | 4 | KAP_TatD | Escherichia coli | pLG037_KAP | Described computationally in |
| | | | NCTC9009 | | Aravind et al. Genome Biol |
| | | | | | (2004) |
| pLG039 | 2 | S8 peptidase | Escherichia coli | pLG039_Protease | Proteasome-like ATPase + |
| | | | ECOR52 | | serine protease |
| pLG041 | 1 | DUF4011 | Escherichia coli | pLG041_DUF4011 | |
| | | | ATCC43886 | | |
| pLG044 | 2 | DUF3684 Hsp90-like | Vibrio harveyi | pLG044_Hsp90 | Large gene (~2500aa) with |
| | | ATPase + helicase | ATCC43516 | | large stretches of unknown |
| | | | | | regions; associated with a |
| | | | | | helicase |
| pLG046 | 3 | Trypsin-AAA35 | Erwinia | pLG046_Protease- | STAND ATPase (these are |
| | | | piriflorinigrans | STAND | not typically thought to be |
| | | | CFBP5888 | | defensive) |
| pLG049 | 2 | DUF4297-AAA3 + | Salmonella enterica | pLG049_DUF4297- | STAND ATPase |
| | | unknown | NCTC13175 | STAND | |
| pLG050 | 1 | DUF4297-AAA35 | Salmonella enterica | pLG050_DUF4297- | STAND ATPase |
| | | | NCTC10718 | STAND | |
| pLG051 | 1 | AAA35 | Escherichia coli | pLG051_STAND | STAND ATPase |
| | | | NCTC9087 | | |
| pLG053 | 1 | RE-AAA35 | Escherichia coli | pLG053_STAND | STAND ATpase |
| | | | NCTC11132 | | |
| pLG056 | 3 | VWA + phosphatase + | Escherichia coli | pLG056_VWA_ | |
| | | kinase | NCTC9094 | phophatase_kinase | |
| pLG061 | 1 | SIR2-DUF4020 | Escherichia coli | pLG061_SIR2- | |
| | | | NCTC9112 | DUF4020 | |
| pLG062 | 1 | SIR2 | Cronobacter sakazakii | pLG062_SIR2 | |
| | | | NCTC8155 | | |
| pLG063 | 1 | SIR2-STAND-TPR | Escherichia coli | pLG063_SIR2- | STAND ATpase |
| | | | NCTC13384 | STAND | |
| pLG066 | 1 | PHP-SMC | Escherichia coli | pLG066_ | |
| | | | NCTC8620 | Phosphoesterase | |
| | | | | (PHP)-SMC | |
| pLG070 | 2 | SIR2 + HerA | Escherichia coli | pLG070_HerA | Modular system (HerA pump |
| | | | NCTC11129 | | can be paired with SIR2, |
| | | | | | DUF4297, etc.) |
| pLG071 | 2 | DUF4297 + HerA | Escherichia coli | pLG070_HerA | Modular system (HerA pump |
| | | | NCTC11131 | | can be paired with SIR2, |
| | | | | | DUF4297, etc.) |
| pLG080 | 1 | Unknown-DUF1887 | Salmonella enterica | pLG080_DUF1887 | ~1200aa gene; first ~1000aa |
| | | | NCTC6026 | | are unknown |
| pLG157 | 2 | DUF262 + | Escherichia coli | — | Described computationally |
| | | DUF262-HNH | ATCC43886 | | in Makarova et al. 2011 |
| pLG078 | 4 | DUF499 + DUF3780 + | Escherichia coli | — | Restriction-modification-like |
| | | DUF1156 | ECOR58 | | system described |
| | | methyltransferase + | | | computationally in |
| | | helicase | | | Anantharaman et al. 2013 |

TABLE 2

| | # genes in | | Donor | Diagram | |
|---|---|---|---|---|---|
| Construct | operon | Short Description | Strain | File Name | Note |
| — | 6 | Type I-E CRISPR- | — | CRISPR_ATPase | Described computationally in Shmakov |
| | | associated | | | et al. PNAS 2017; predicted to be non- |
| | | | | | defense |
| — | 1 | RT-protease | — | RT-protease | Retron; described computationally in |
| | | | | | Zimmerly & Wang (2015) |

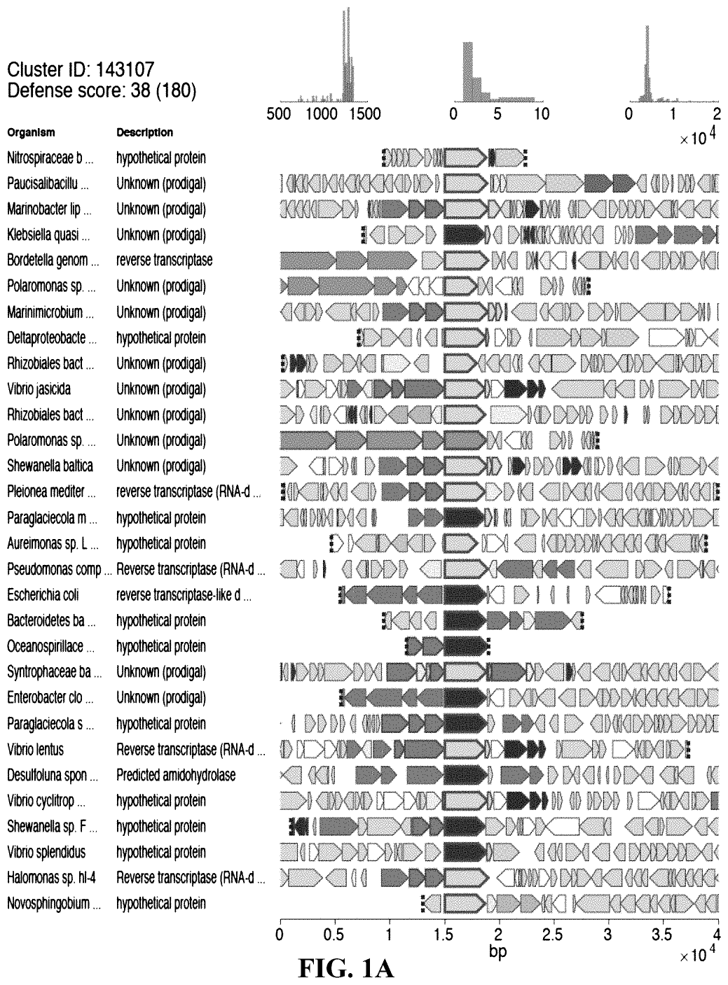

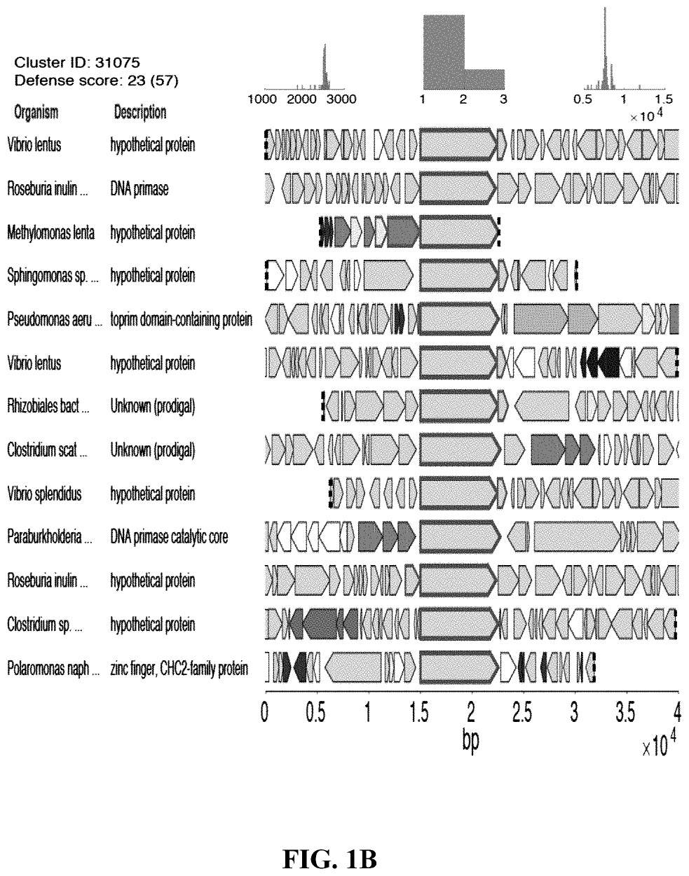

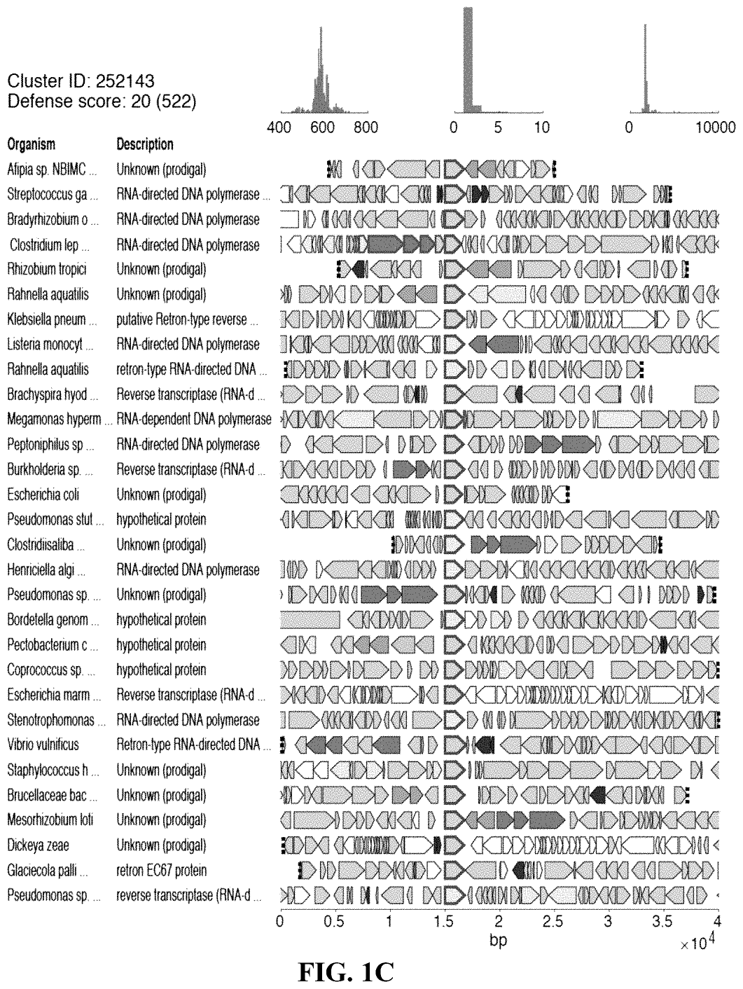

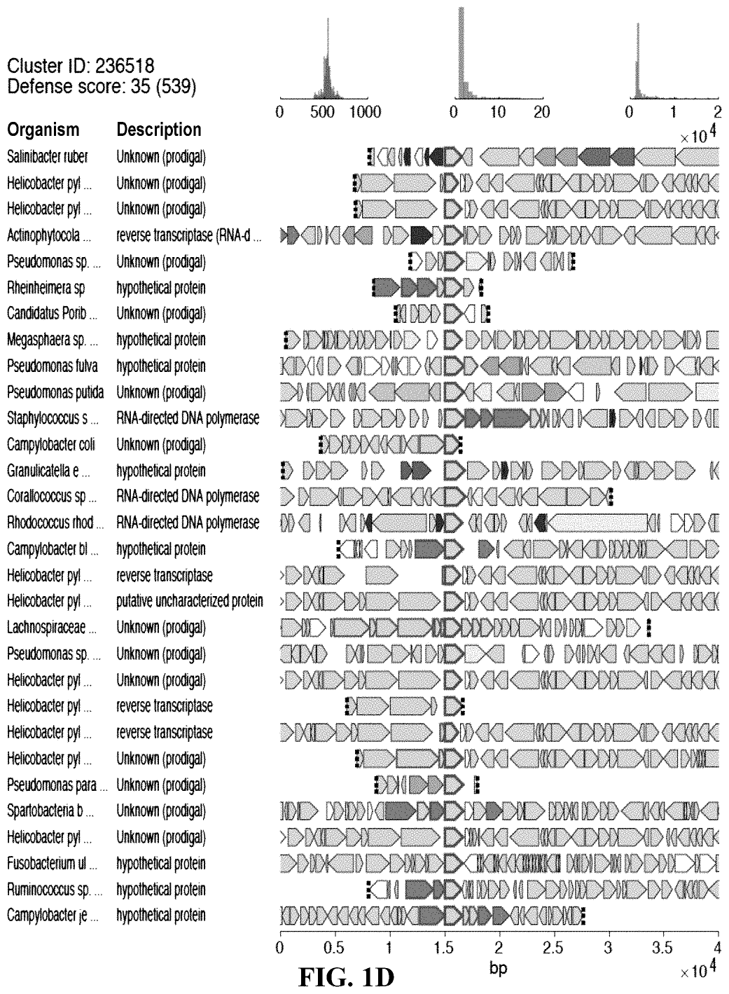

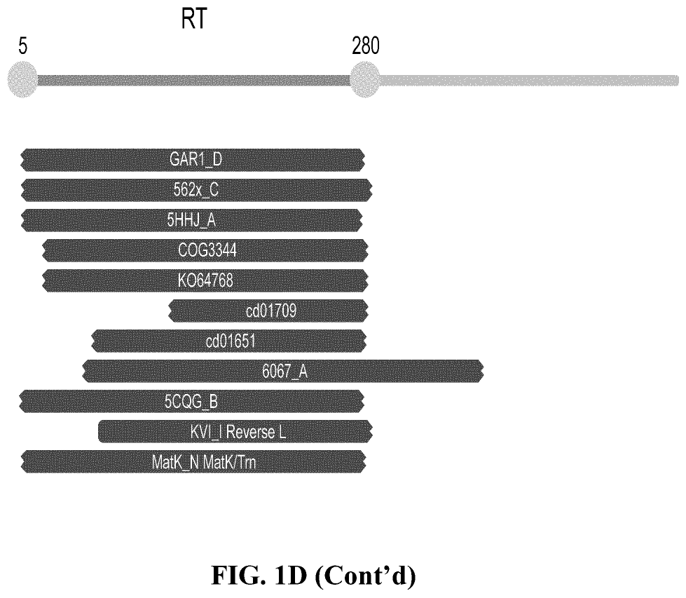

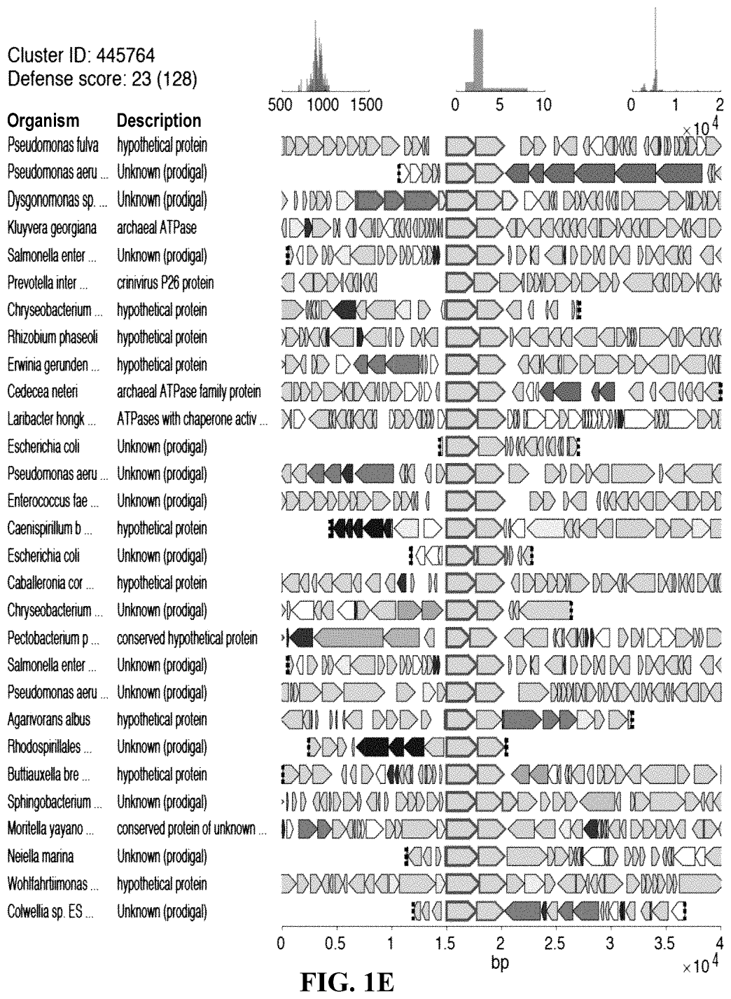

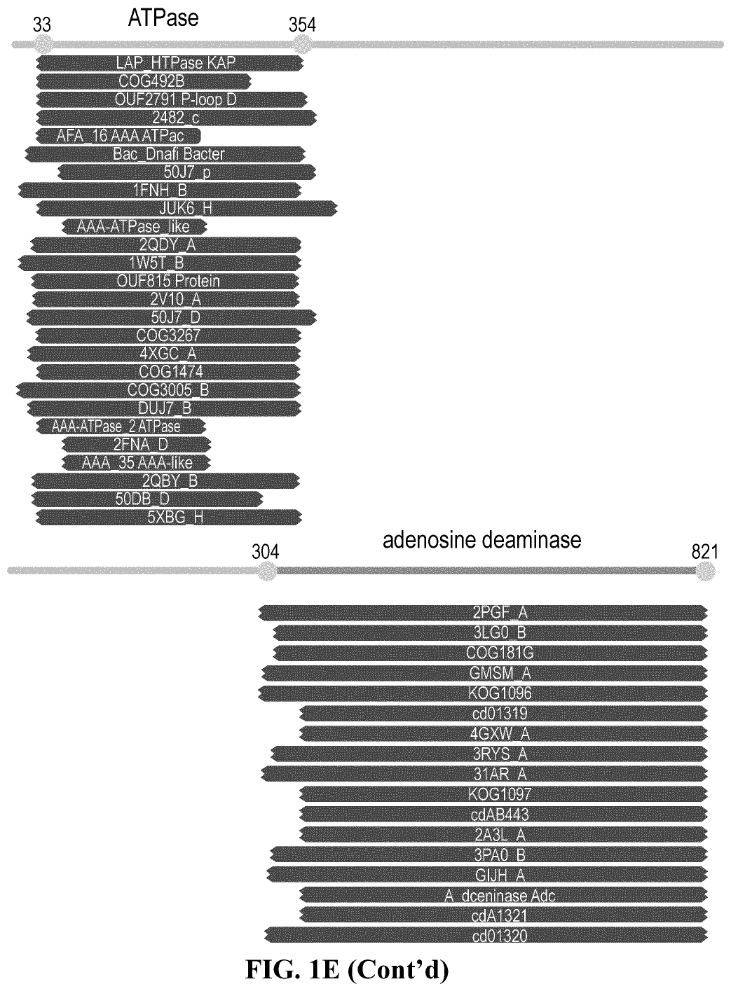

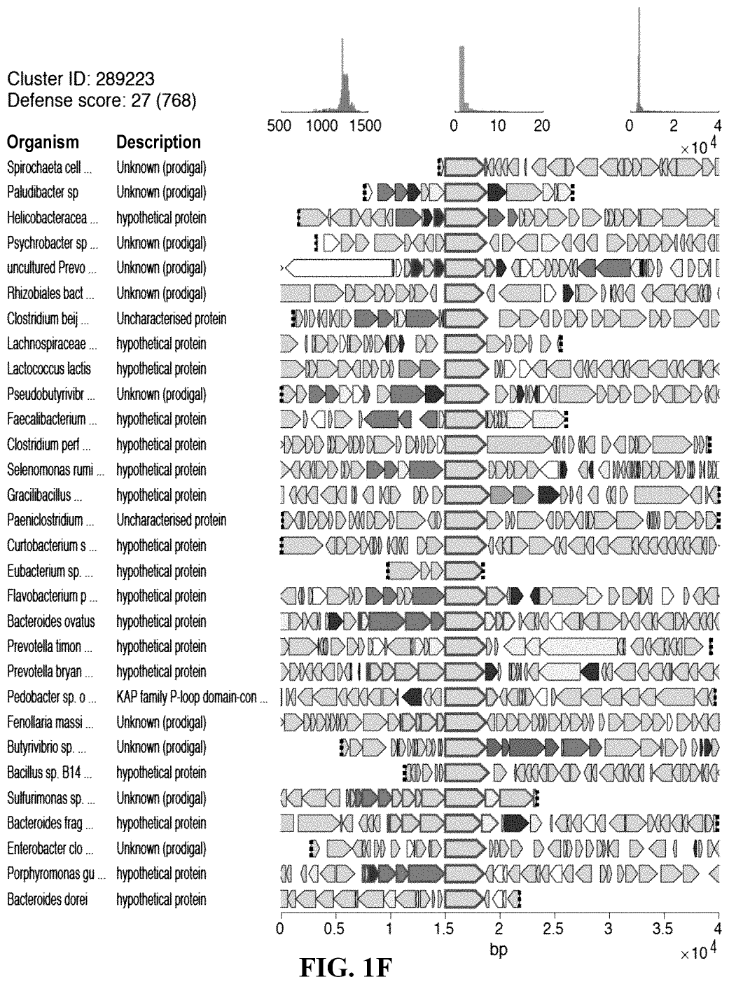

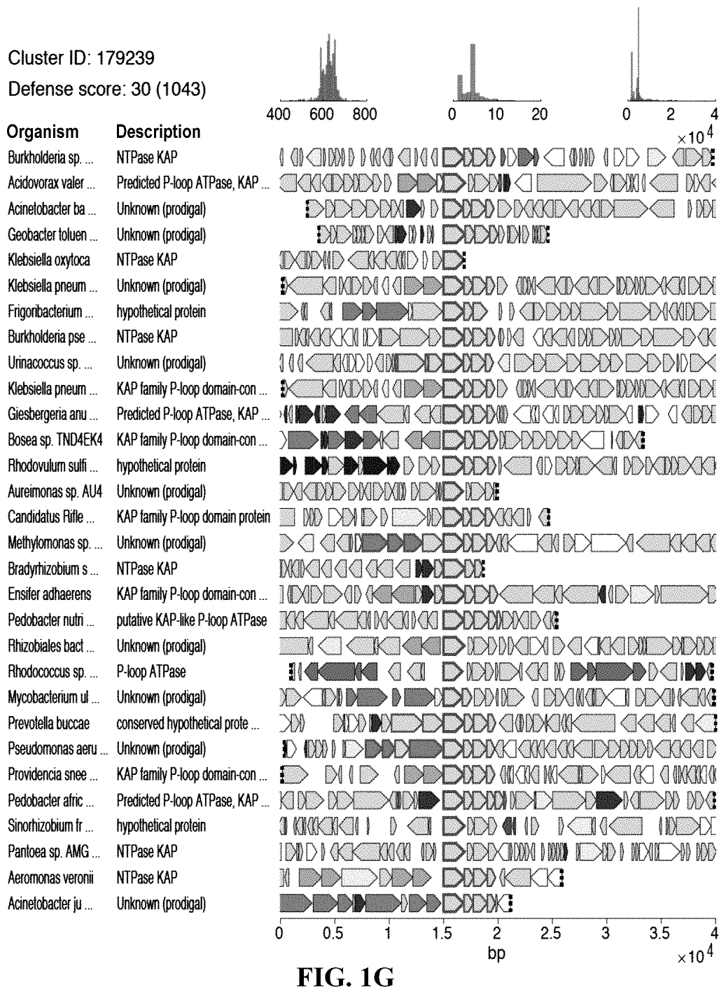

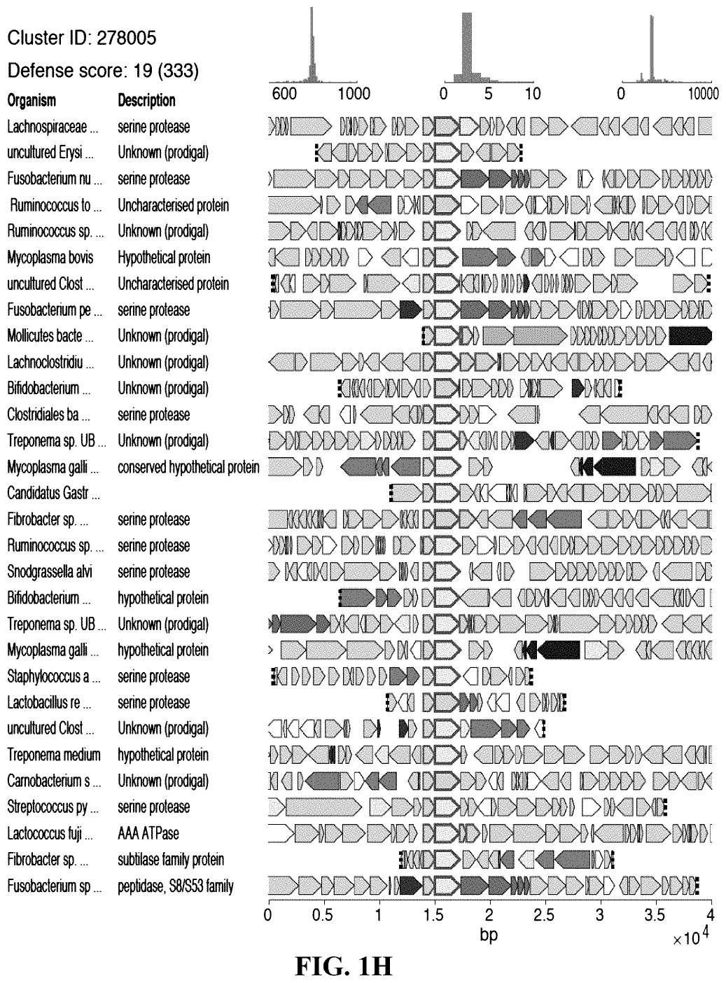

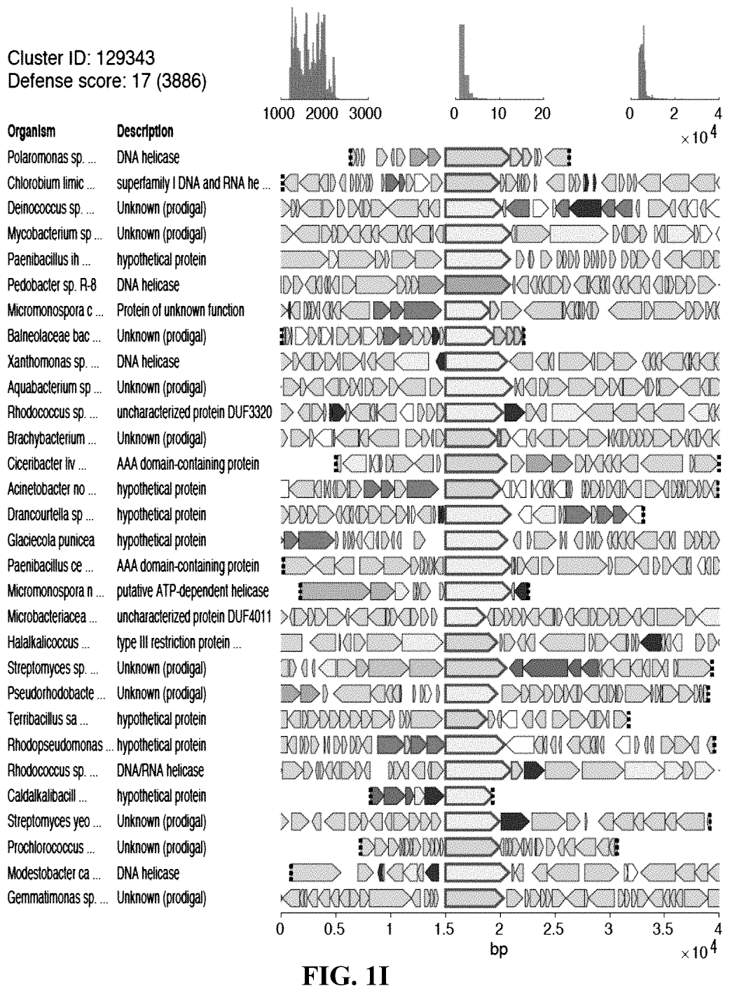

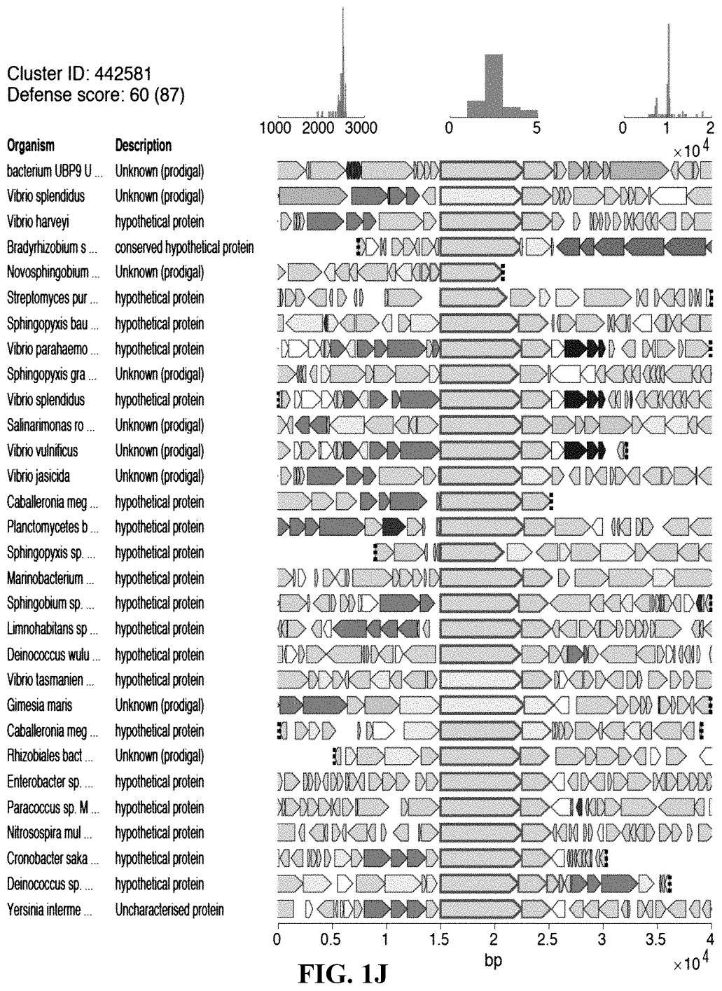

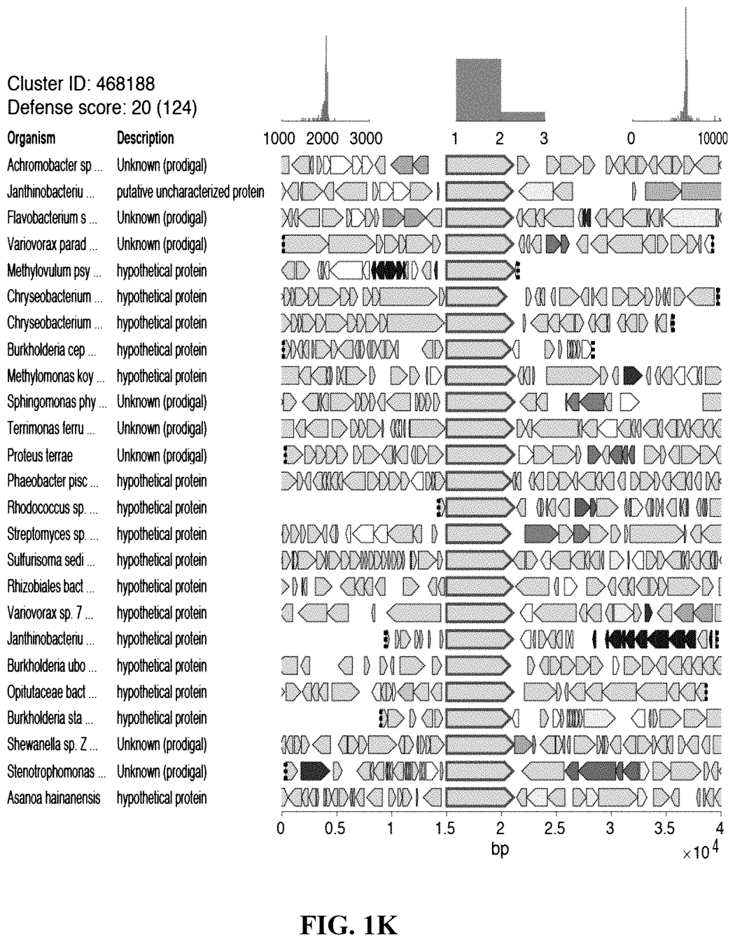

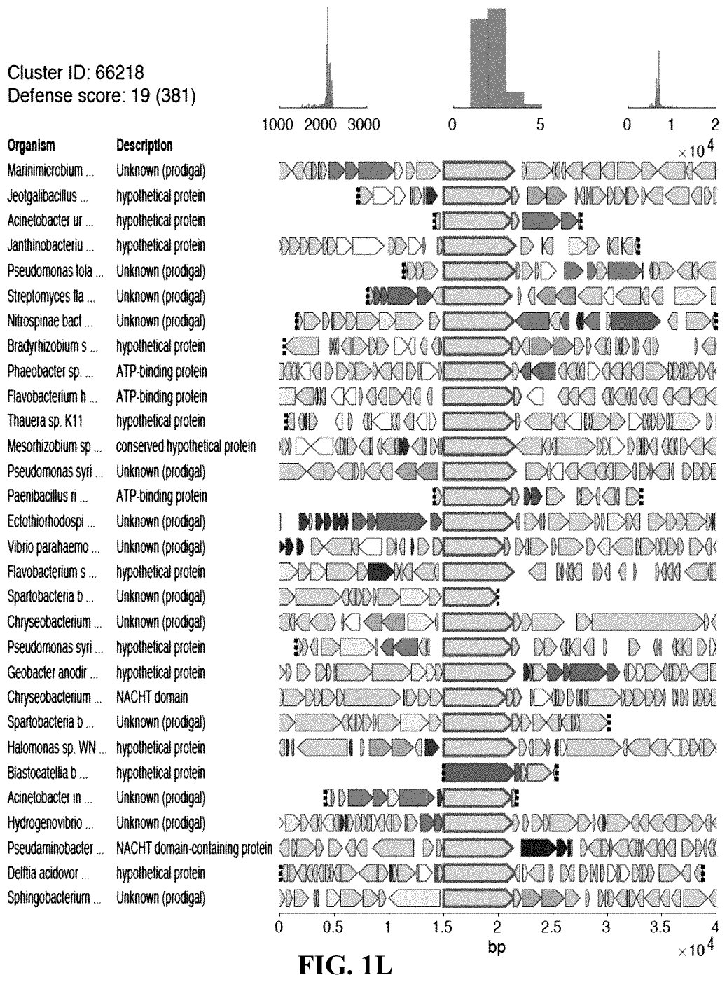

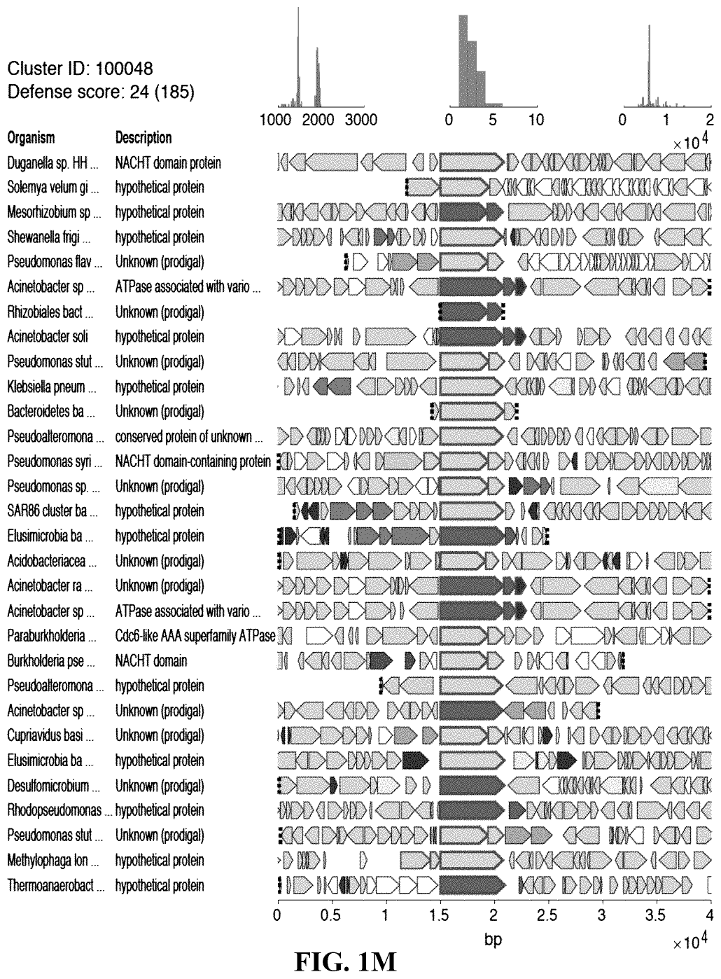

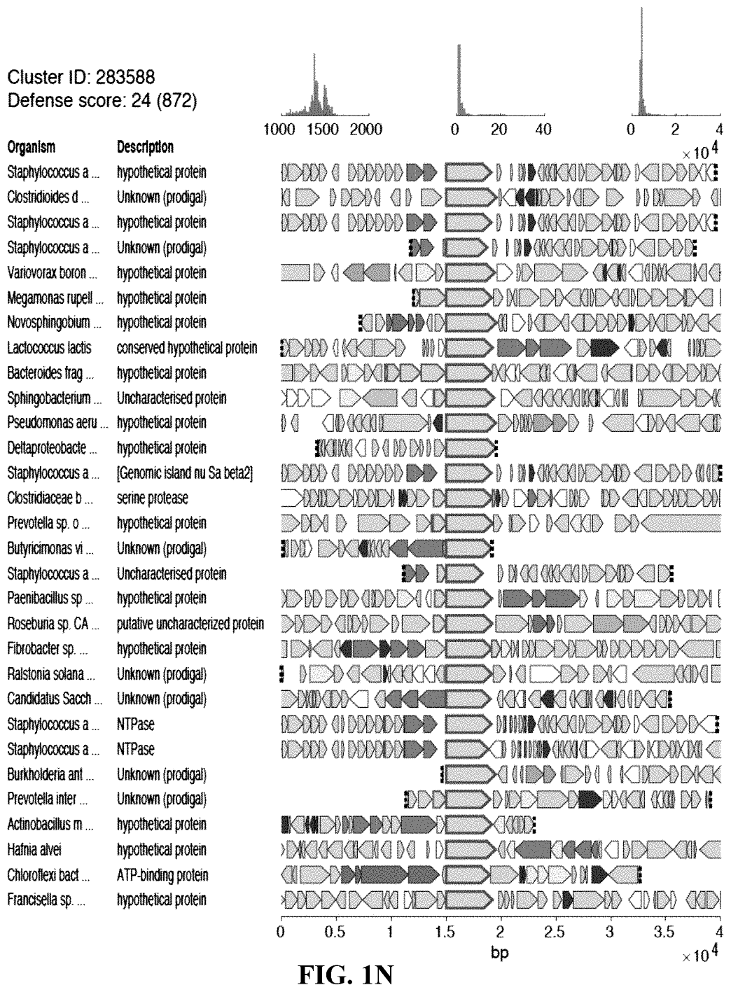

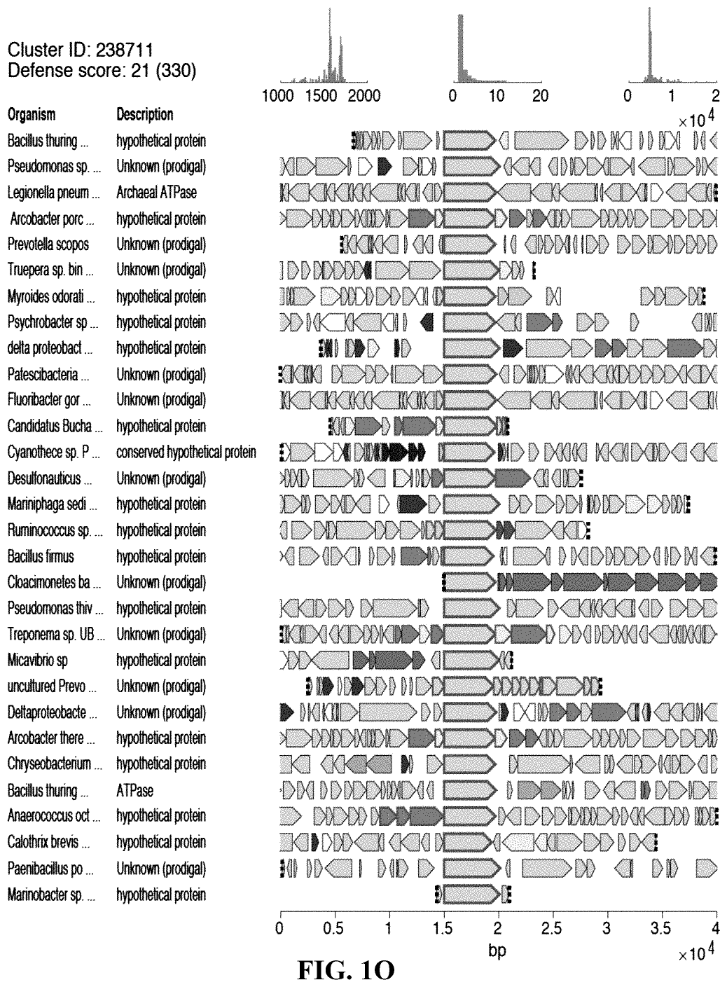

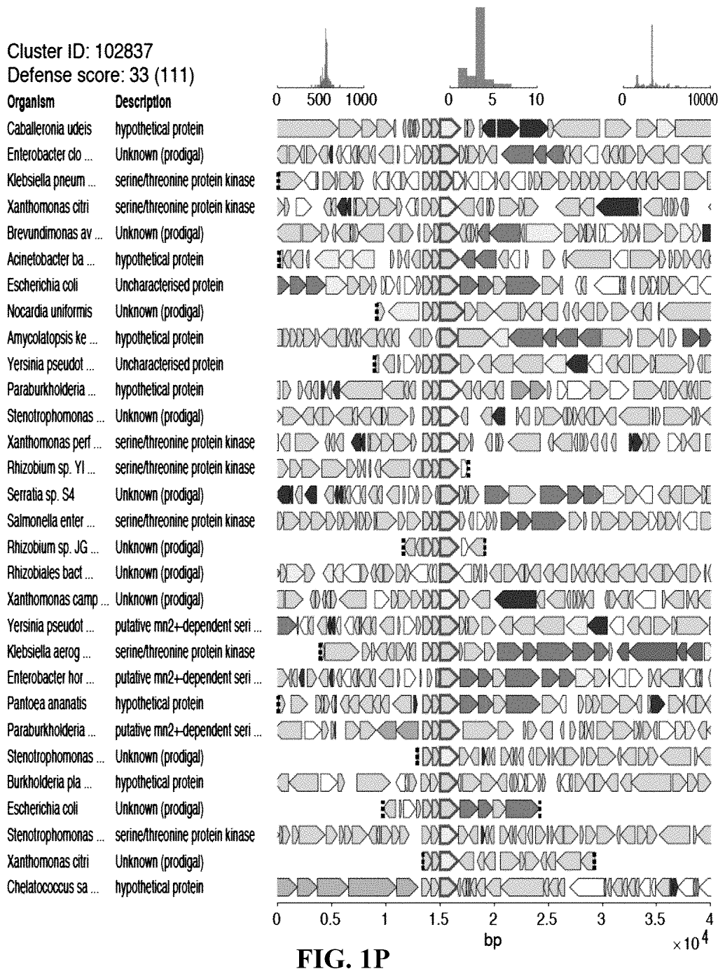

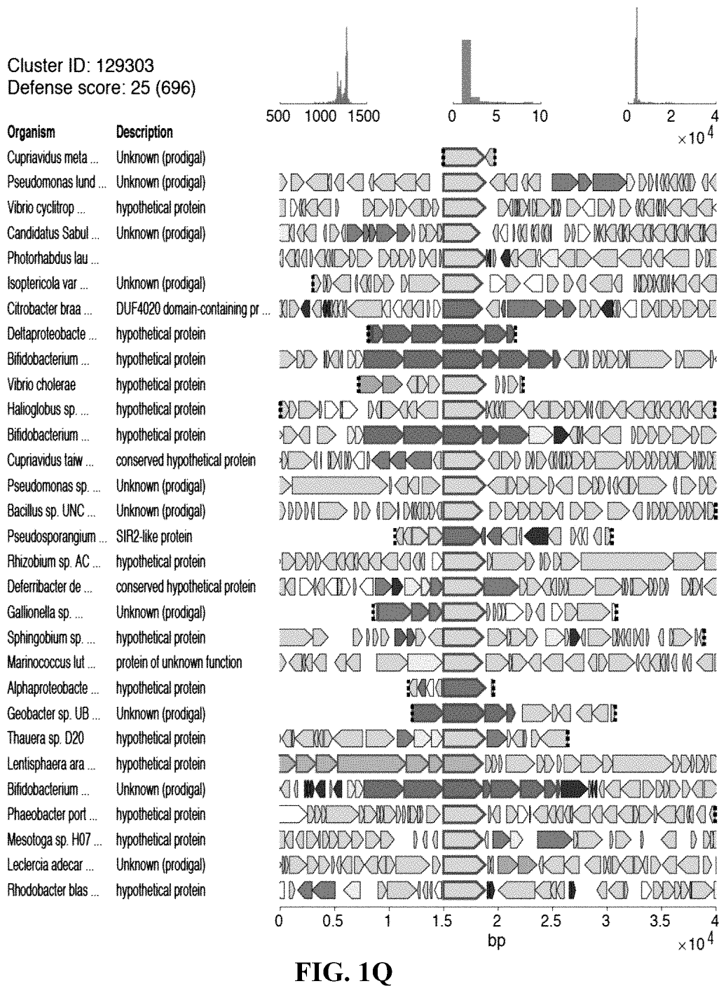

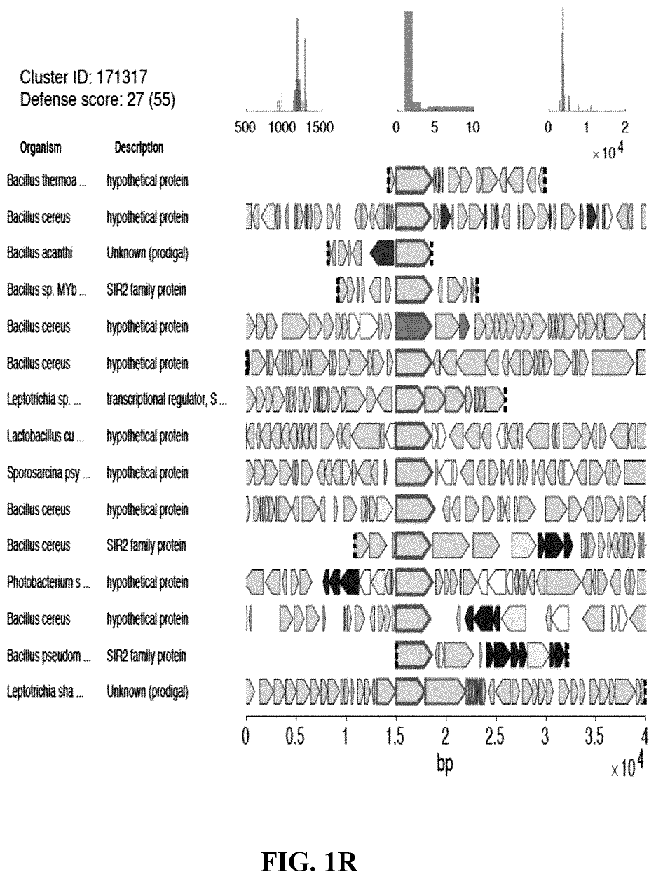

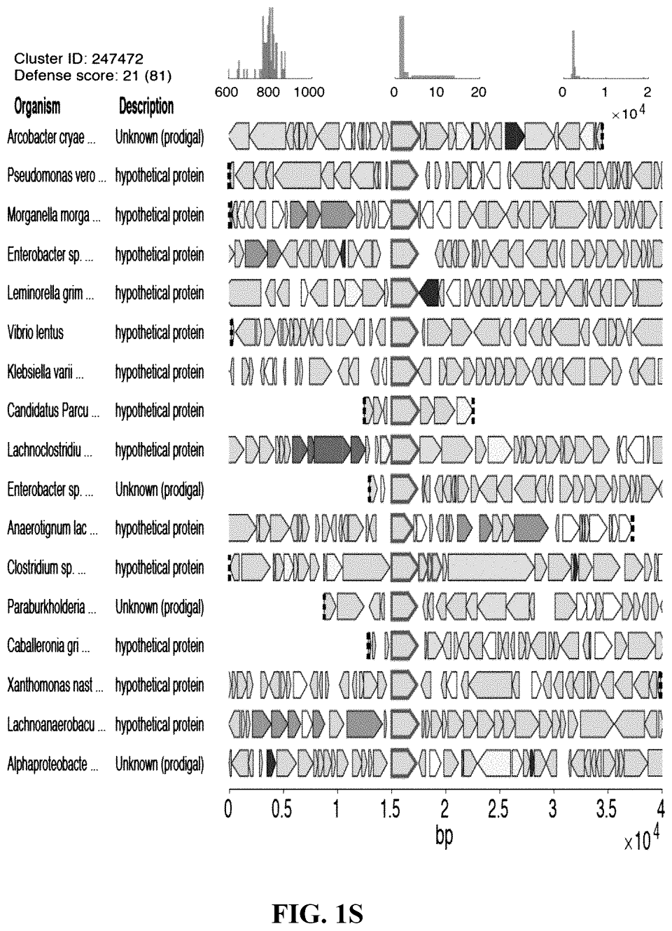

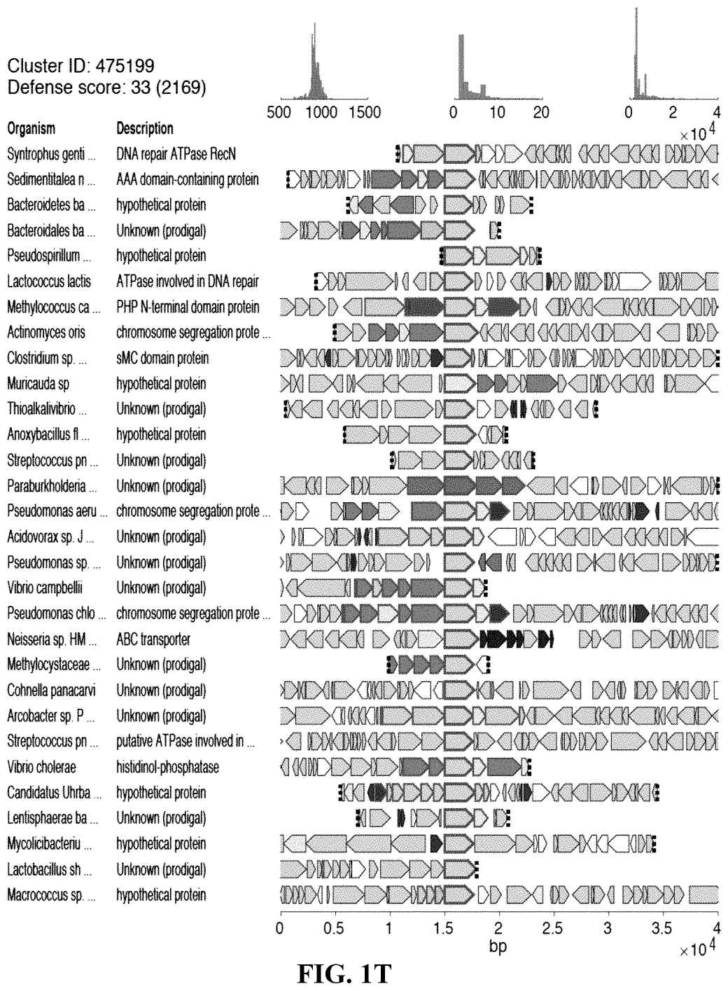

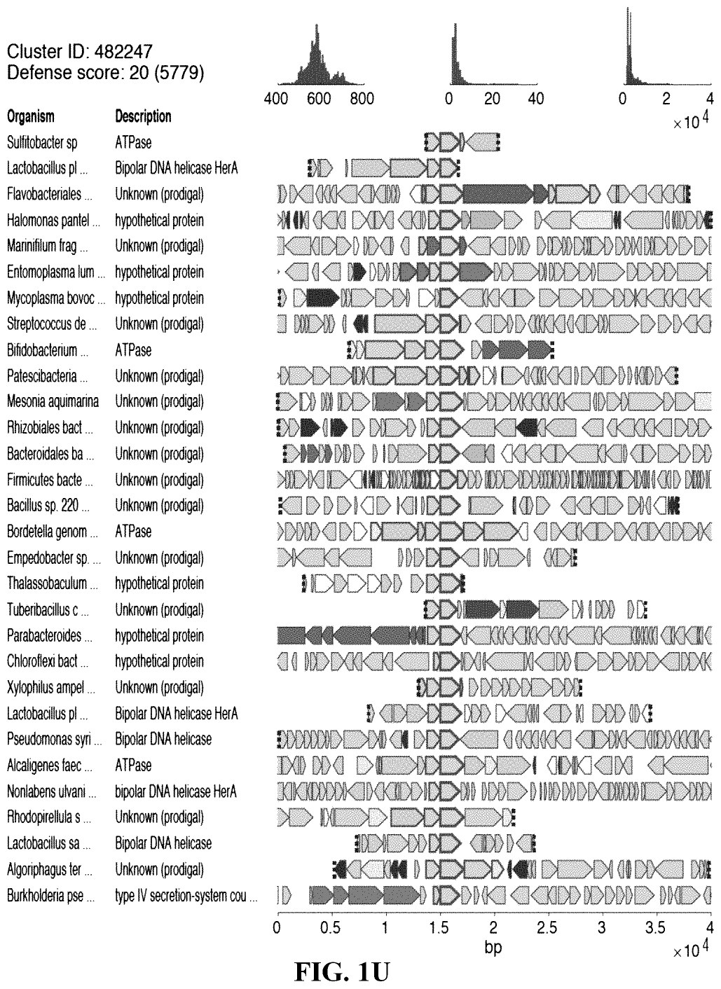

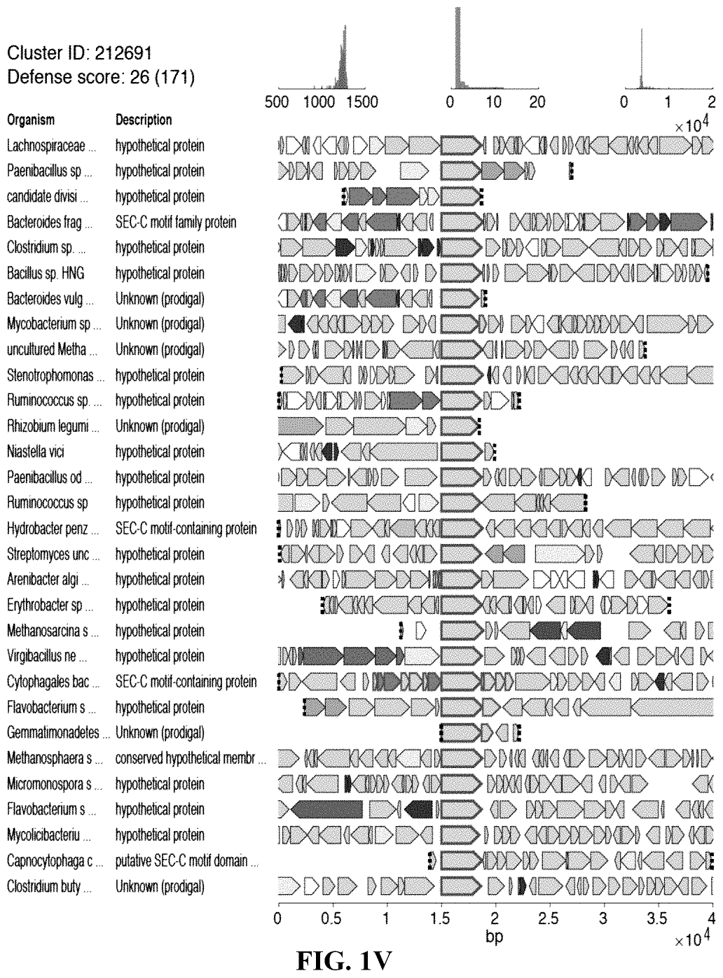

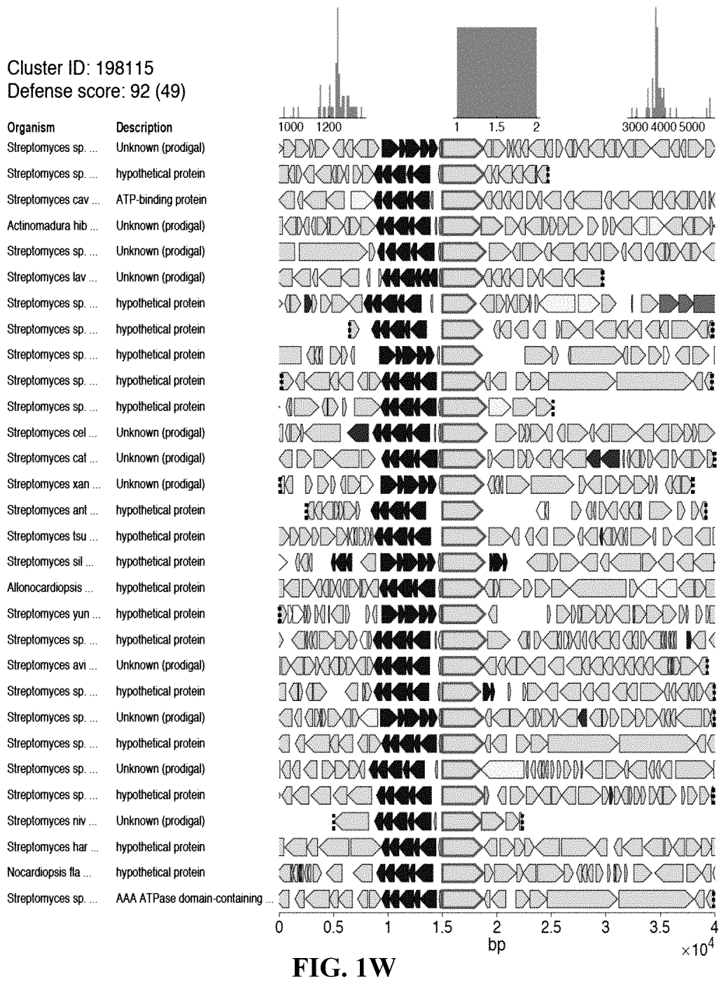

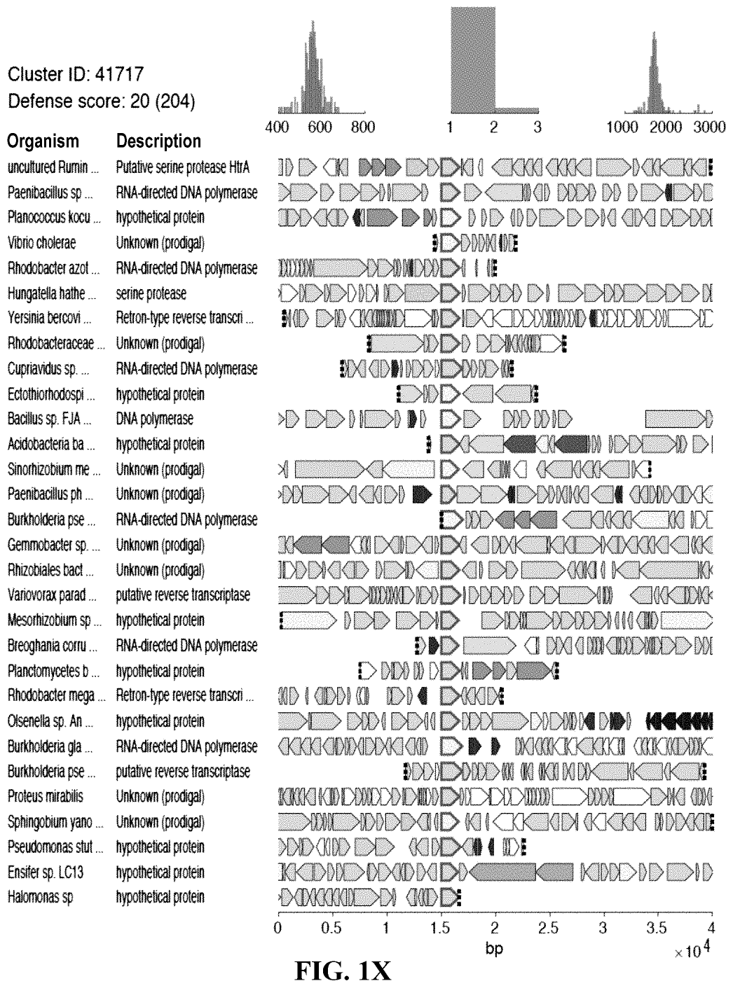

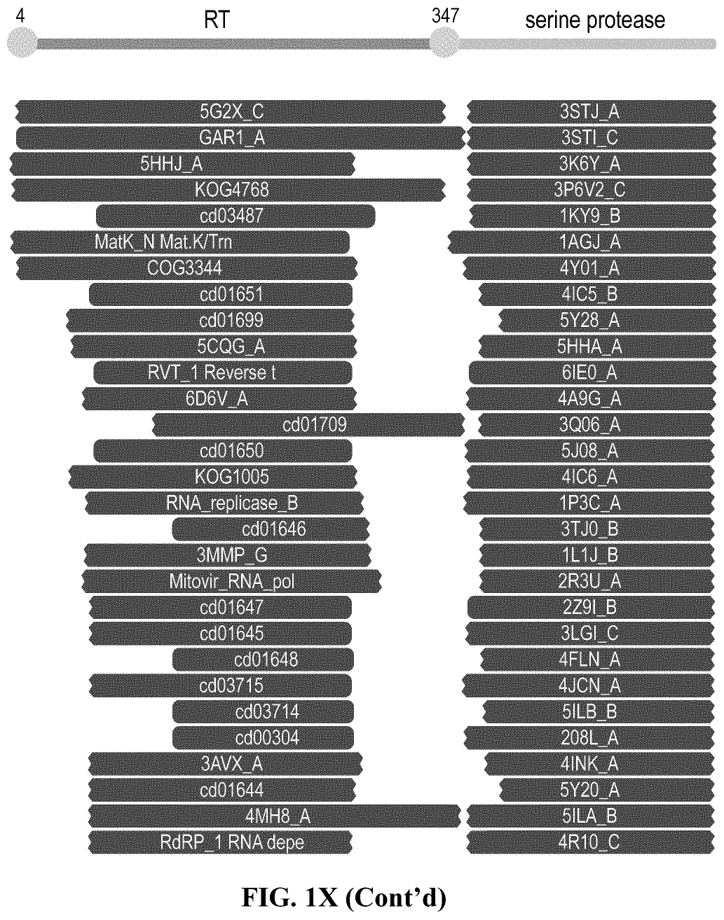

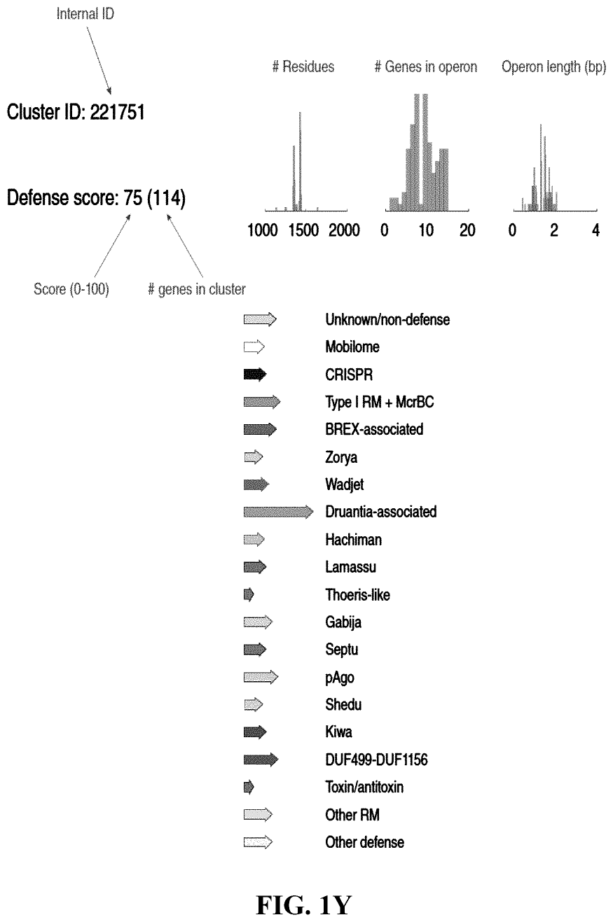

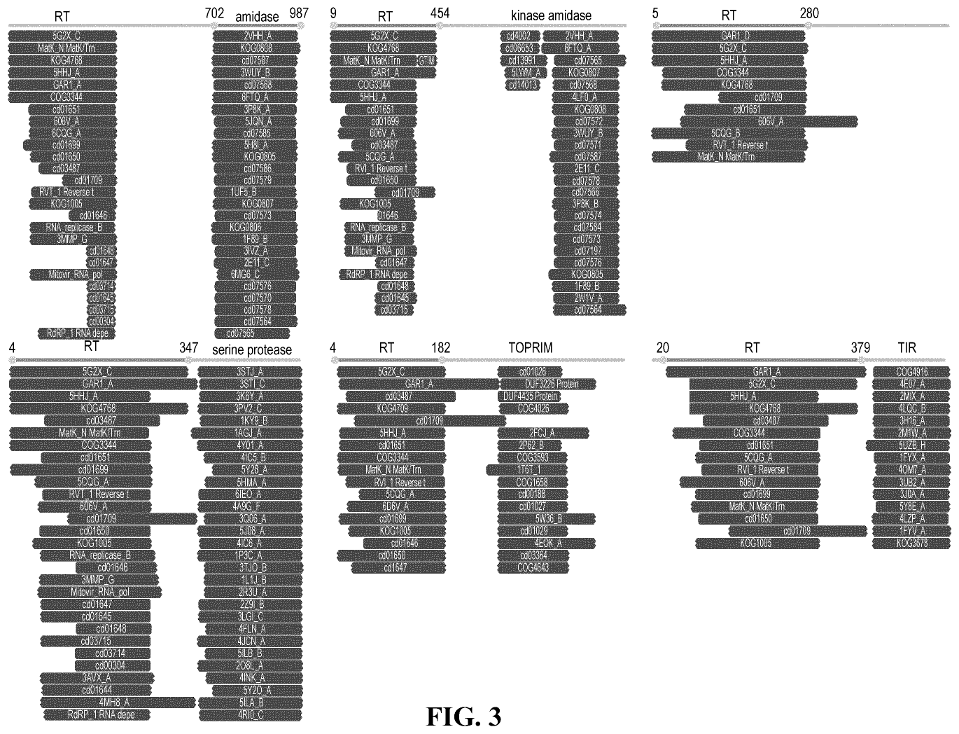

FIGS. 1A-1Y, 2, and 3 show diagrams of domain structures of exemplary defense systems.

### Additional Exemplary Systems

Additional examples of systems are shown in Tables 3A-3B below.

TABLE 3A

| Row | | | | # | | | | | |
|---|---|---|---|---|---|---|---|---|---|
| No. | Vector | System | System details | genes | Organism | Strain | bp | Note | Source |
| 1 | pLG003 | Control | BREX type I | 6 | E. coli | NCTC9078 | 13703 | Goldfarb et al. | |
| | | | | | | (DSM5212) | | 2014 | |
| 2 | pLG004 | Control | Druantia type I | 5 | E. coli | NCTC9078 | 11823 | Doron et al. | |
| | | | | | | (DSM5212) | | Science 2018 | |
| 3 | pLG005 | Control | Type I RM | 3 | E. coli | NCTC13846 | 6946 | | bloodculture, |
| | | | | | | (DSM105182) | | | human |
| | | | | | | | | | bacteraemia, |
| | | | | | | | | | UK |
| 4 | pLG006 | Control | Zorya type II | 3 | E. coli | ATCC8739 | 3917 | Doron et al. | Feces |
| | | | | | | | | Science 2018 | |
| 5 | pLG007 | Control | RT-AbiA | 1 | E. coli | ECOR30 | 1921 | Odegrip et al. | Bison, Alberta, |
| | | | | | | (ATCC35349) | | 2006 | Canada |
| 6 | pLG008 | Control | RT-AbiK | 1 | Lactococcus | W-1 | 2102 | Wang et al. | |
| | | | | | lactis | | | NAR 2011 | |
| 7 | pLG009 | RT | RT-protease | 1 | Stenotrophomonas | TG_2005 | | | |
| | | | | | maltophilia | | | | |
| 8 | pLG010 | RT | RT-protease | 1 | Haematobacter | KC2145 | | | |
| | | | | | massiliensis | | | | |
| 9 | pLG011 | RT | RT-protease | 1 | Sphingobium | ATCC51230 | 2029 | | clinical |
| | | | | | yanoikuyae | (DSM7462) | | | specimen |
| 10 | pLG012 | RT | RT-protease | 1 | Proteus mirabilis | 127_PMIR | 2009 | | |
| 11 | pLG013 | RT | RT-protease | 1 | Pseudomonas | PA-W9 | | | |
| | | | | | aeruginosa | | | | |
| 12 | pLG014 | RT | RT-protease | 1 | Photobacterium | NCTC11646 | 2657 | | human, leg |
| | | | | | damselae | | | | wound |
| 13 | pLG015 | RT | RT-protease | 1 | Paraburkholderia | PSCR-88 | | | |
| | | | | | silvatlantica | | | | |
| 14 | pLG016 | RT | RT-protease | 1 | Bacillus subtilis | ATCC13952 | 2203 | | |
| 15 | pLG017 | RT | RT-kinase- | 1 | E. coli | N1 | 4154 | | |
| | | | nitrilase | | | | | | |
| 16 | pLG018 | RT | RT-kinase- | 1 | Klebsiella | NCTC9143 | 5272 | SLATT | Urine |
| | | | nitrilase | | pneumoniae | | | associated | |
| 17 | pLG019 | RT | RT-nitrilase | 1 | E. coli | NCTC4169 | 3679 | | human, excreta |
| 18 | pLG020 | RT | RT-nitrilase | 1 | Klebsiella | KPNIH39 | 3479 | | uterine |
| | | | | | pneumoniae | | | | secretion |
| 19 | pLG021 | RT | TOPRIM-RT- | 1 | Pseudomonas | DSM16299 | 8446 | | rhizosphere |
| | | | nitrilase | | rhizosphaerae | | | | of grasses |
| 20 | pLG108 | RT | TOPRIM-RT- | 1 | Vogesella | DSM3303 | | | Garden soil, |
| | | | nitrilase | | indigofera | | | | Pacific Grove |
| | | | | | | | | | California |
| 21 | pLG023 | RT | RT-TIR | 1 | E. coli | NCTC9024 | 2393 | | |
| 22 | pLG024 | RT | RT-TIR | 1 | Shigella | NCTC2966 | 2139 | | monkey with |
| | | | | | dysenteriae | | | | enteritis |
| 23 | pLG025 | RT | RT-TOPRIM | 1 | E. coli | NCTC13441 | 2569 | | |
| 24 | pLG026 | RT | RT-TOPRIM | 1 | E. coli | NCTC8623 | 2405 | | gastro- |
| | | | | | | | | | enteritis |
| 25 | pLG027 | RT | RT-345 | 1 | E. coli | STEC 66 | 1951 | | |
| 26 | pLG028 | RT | RT-345 | 1 | E. coli | 21-C8-A | 2141 | | |
| 27 | pLG029 | RT | RT-x2 | 2 | E. coli | NCTC9091 | 3648 | | |
| 28 | pLG030 | RT | RT-x2 | 3 | Acinetobacter | NCTC7412 | 4236 | SLATT | human, urine |
| | | | | | calcoaceticus | | | associated | |
| 29 | pLG031 | ADA | Adenosine | 2 | E. coli | NCTC11116 | 5533 | | |
| | | | deaminase | | | | | | |
| 30 | pLG032 | ADA | Adenosine | 2 | Citrobacter | ATCC51459 | 5526 | | Laboratory |
| | | | deaminase | | rodentium | | | | mouse |
| 31 | pLG033 | ADA | Adenosine | 3 | Pluralibacter | ATCC33028 | 6689 | SLATT | Urine, France |
| | | | deaminase | | gergoviae | | | associated | |
| 32 | pLG034 | KAP | Transmembrane | 1 | E. coli | ECOR25 | 4415 | | Dog, New York |
| | | | KAP ATPase | | | (ATCC35344) | | | |
| 33 | pLG035 | KAP | Transmembrane | 1 | E. coli | NCTC8620 | 4037 | | human, diarrhoea |
| | | | KAP ATPase | | | | | | |
| 34 | pLG036 | KAP | KAP + | 4 | E. coli | ECOR10 | 4891 | | Adult human, |
| | | | unknown + | | | (ATCC35329) | | | New York |
| | | | QueC + TatD | | | | | | |
| 35 | pLG037 | KAP | KAP + | 4 | E. coli | NCTC9009 | 5408 | | |
| | | | unknown + | | | | | | |
| | | | QueC + TatD | | | | | | |
| 36 | pLG038 | Protease | ATPase + | 2 | E. coli | ECOR12 | 3678 | | Adult human, |
| | | | serine protease | | | (ATCC35331) | | | Sweden |
| 37 | pLG039 | Protease | ATPase + | 2 | E. coli | ECOR52 | 3676 | | Orangutan, |
| | | | serine protease | | | (ATCC35371) | | | Seattle Zoo, |
| | | | | | | | | | Washington |
| 38 | pLG040 | Protease | ATPase + | 2 | E. coli | NCTC9008 | 3917 | | pathogenic |
| | | | serine protease | | | | | | to chicks |
| 39 | pLG041 | DUF4011 | DUF4011- | 1 | E. coli | ATCC43886 | 5958 | | Feces, human |
| | | | helicase-Vsr- | | | | | | |
| | | | DUF3320 | | | | | | |
| 40 | pLG042 | DUF4011 | DUF4011- | 1 | Citrobacter | NCTC9067 | 6502 | | |
| | | | helicase-Vsr- | | braakii | | | | |
| | | | DUF3320 | | | | | | |
| 41 | pLG043 | DUF3684 | Hsp90-like | 2 | Pectobacterium | CFBP3304 | 10581 | | Japanese |
| | | | ATPase + | | wasabiae | (ATCC43316) | | | horseradish, |
| | | | SNF2 | | | | | | Eutrema wasabi, |
| | | | | | | | | | Japan |
| 42 | pLG044 | DUF3684 | Hsp90-like | 2 | Vibrio harveyi | ATCC43516 | 10687 | | Mouth of |
| | | | ATPase + | | | | | | shark, Bahamas |
| | | | SNF2 | | | | | | |
| 43 | pLG045 | DUF3684 | Hsp90- | 1 | Raoultella | NCTC9528 | 5918 | | butter |
| | | | DUF3684- | | planticola | | | | |
| | | | DUF3883- | | | | | | |
| | | | PDDEXK(CTD) | | | | | | |
| 44 | pLG046 | AAA35 | Protease- | 3 | Erwinia | CFBP 5888 | 7847 | | necrotic |
| | | | AAA35 | | piriflorinigrans | (DSM26166) | | | pear blossoms, |
| | | | | | | | | | Valencia, Spain |
| 45 | pLG047 | AAA35 | Protease- | 3 | Pectobacterium | M022 | 7740 | | |
| | | | AAA35 | | fontis | (LMG30744) | | | |
| 46 | pLG048 | AAA35 | DUF4297- | 1 | E. coli | NCTC9036 | 6514 | | |
| | | | AAA35-TPR | | | | | | |
| 47 | pLG049 | AAA35 | DUF4297- | 2 | Salmonella | NCTC13175 | 7175 | | |
| | | | AAA35 | | enterica | | | | |
| 48 | pLG050 | AAA35 | DUF4297- | 1 | Salmonella | NCTC10718 | 6261 | | |
| | | | AAA35 | | enterica | | | | |
| 49 | pLG051 | AAA35 | Unknown- | 1 | E. coli | NCTC9087 | 5109 | | |
| | | | AAA35- | | | | | | |
| | | | unknown | | | | | | |
| 50 | pLG052 | AAA35 | Unknown- | 1 | E. coli | NCTC10650 | 4781 | | |
| | | | AAA35- | | | | | | |
| | | | unknown | | | | | | |
| 51 | pLG053 | AAA35 | RE-AAA35 | 1 | E. coli | NCTC11132 | 4964 | | |
| 52 | pLG054 | Kinase | DUF2357 | 7 | Obesumbacterium | DSM2777 | 12191 | | ale yeast |
| | | | | | proteus | | | | |
| 53 | pLG055 | Kinase | Kinase- | 2 | E. coli | NCTC13919 | 6873 | | Clinical isolate. |
| | | | helicase_1600aa | | | | | | Human, rectum |
| 54 | pLG056 | Kinase | VWA + | 3 | E. coli | NCTC9094 | 3605 | | |
| | | | phosphatase + | | | | | | |
| | | | kinase | | | | | | |
| 55 | pLG057 | Kinase | 5-gene McrBC- | 5 | Plasticicumulans | DSM25287 | 11931 | | lactate-fed |
| | | | like | | lactativorans | | | | bioreactor |
| | | | | | | | | | inoculated with |
| | | | | | | | | | activated sludge |
| | | | | | | | | | from a sewage |
| | | | | | | | | | treatment plant, |
| | | | | | | | | | Kralingseveer, |
| | | | | | | | | | Rotterdam, |
| | | | | | | | | | Netherlands |
| 56 | pLG058 | GTPase | GTPase | 3 | Pantoea | LMG 2657 | 4789 | | cypripedium orchid, |
| | | | | | cypripedii | (DSM3873) | | | California |
| 57 | pLG059 | GTPase | GTPase | 3 | Pectobacterium | CFBP3304 | 5216 | | Japanese |
| | | | | | wasabiae | (ATCC43316) | | | horseradish, |
| | | | | | | | | | Eutrema wasabi, |
| | | | | | | | | | Japan |
| 58 | pLG060 | GTPase | GTPase | 3 | E. coli | NCTC10962 | 4577 | | faeces(arabian |
| | | | | | | | | | gulf) |
| 59 | pLG061 | SIR2 | SIR2-DUF4020 | 1 | E. coli | NCTC9112 | 4212 | | |
| 60 | pLG062 | SIR2 | SIR2-TPR- | 1 | Cronobacter | NCTC8155 | 4329 | | tin of dried |
| | | | HEAT | | sakazakii | | | | milk |
| 61 | pLG063 | SIR2 | SIR2-AAA35 | 1 | E. coli | NCTC13384 | 3411 | | |
| | | | | | | (ATCC11229) | | | |
| 62 | pLG064 | Misc | Dcm + | 5 | Pseudomonas | NCTC10727 | 11911 | | |
| | | | unknown + | | aeruginosa | | | | |
| | | | unknown + | | | | | | |
| | | | HerA + Vsr | | | | | | |
| 63 | pLG065 | Misc | Dcm + | 5 | Aquimonas voraii | DSM16957 | 11635 | | water, |
| | | | unknown + | | | | | | Assam, India |
| | | | unknown + | | | | | | |
| | | | HerA + Vsr | | | | | | |
| 64 | pLG066 | Misc | Phosphoesterase | 1 | E. coli | NCTC8620 | 3066 | | human, diarrhoea |
| | | | (PHP)-SMC | | | | | | |
| 65 | pLG067 | Misc | Helicase- | 2 | E. coli | NCTC9033 | 7356 | | |
| | | | nuclease_unknown | | | | | | |
| 66 | pLG068 | Misc | DUF3893 | 3 | Pseudomonas | DSM10604 | 6714 | | common lilac |
| | | | (possible pAgo) | | syringae | | | | |
| 67 | pLG069 | Misc | RecQ | 1 | Klebsiella | NCTC11696 | 5424 | | |
| | | | | | oxytoca | | | | |
| 68 | pLG070 | Misc | SIR2 + HerA | 2 | E. coli | NCTC11129 | 3308 | | |
| 69 | pLG071 | Misc | DUF4297 + | 2 | E. coli | NCTC11131 | 3419 | | |
| | | | HerA | | | | | | |
| 70 | pLG072 | Misc | Dcm + Hsp90- | 4 | E. coli | NCTC86 | 7655 | | |
| | | | sensor histidine | | | (DSM301) | | | |
| | | | kinase + | | | | | | |
| | | | response regulator | | | | | | |
| 71 | pLG073 | Misc | Dcm + Hsp90- | 4 | E. coli | NCTC11560 | 6042 | | |
| | | | sensor histidine | | | | | | |
| | | | kinase + | | | | | | |
| | | | response regulator | | | | | | |
| 72 | pLG074 | Misc | Palatin + | 4 | Klebsiella | NCTC9735 | 4755 | | |
| | | | nucleotidyltrans- | | aerogenes | | | | |
| | | | ferase + | | | | | | |
| | | | UBCc/ThiF + | | | | | | |
| | | | ubiquitin-like | | | | | | |
| 73 | pLG075 | Misc | Sensor histidine | 2 | Pseudomonas | NCTC13717 | 4088 | | |
| | | | kinase + | | aeruginosa | | | | |
| | | | phosphoribosyltrans- | | | | | | |
| | | | ferase | | | | | | |
| 74 | pLG076 | Misc | PH-TerB- | 2 | Klebsiella | NCTC11357 | 3637 | | |
| | | | DUF726 | | pneumoniae | | | | |
| | | | (transmembrane) + | | | | | | |
| | | | Nup (transmembrane) | | | | | | |
| 75 | pLG077 | Misc | TerB- | 3 | E. coli | NCTC9024 | 6037 | Identified in | |
| | | | DUF2791-Lhr | | | | | Doron et al. | |
| | | | | | | | | Science 2018 | |
| 76 | pLG078 | Misc | DUF499 + | 3 | E. coli | ECOR58 | 9809 | Identified in | Lion, |
| | | | DUF1156 | | | (ATCC35377) | | Anantharaman | Seattle Zoo, |
| | | | | | | | | et al. Biology | Washington |
| | | | | | | | | Direct 2013, | |
| | | | | | | | | 8: 15 | |
| 77 | pLG079 | Kinase | 5-gene McrBC- | 5 | Yoonia | DSM29955 | 11425 | | tidal flat |
| | | | like | | sediminilitoris | | | | sediment, |
| | | | | | | | | | South Korea |
| 78 | pLG080 | Misc | DUF1887 | 1 | Salmonella | NCTC6026 | 4100 | | |
| | | | CTD; no other | | enterica | | | | |
| | | | domains | | | | | | |

TABLE 3B
Sequences of loci of row numbers 1-78 of Table 3A.

| Row | | |
|---|---|---|
| No. | Vector | Locus |
| 1 | pLG003 | acagcaccacgttcatcttccttttttaactgattttacagagactttaatacagttaaaattttatttcctgagctgtaatcgat |
| | | taagttgatgcatttaatgggaatgatatagggtcatttccagtctcacttatagaaatggctaaagcatgactctcgccaaaacc |
| | | gtttatgtgttgtacataacgcgatcatccctctcacaaattgccttttctcatggcatctcgcccggtcccccattacaatcact |
| | | ttttgttttttgcgagctgcattccagtcttcagagggtttttcgatgattaaaaatgacaaggcatggataggagacttgctggg |
| | | cggaccgctcatgagcagggaaagccgcgtcattgccgaactgttgctaaccgatcccgatgaacagacatggcaagagcaaattg |
| | | ttggccacaacattttacaagcctcttctcctaacaccgcaaaacgttacgcggcaacaatcaggcttcgcctgaacacgctggat |
| | | aaaagcgcgtggacattgattgccgaaggtagtgaacgggaacgccaacaacttctgtttgtggctctgatgctacattcgccggt |
| | | agttaaggattttctggctgaagtggtgaacgatctgcgcaggcagttcaaggaaaagttgcctggcaatagctggaacgaatttg |
| | | tgaatagccaggttcgcctacatccggtactcgccagctactcagattcatctattgcaaaaatgggaaacaatctggtgaaggcg |
| | | cttgctgaagcgggttatgtggatacgccccgcagacgtaacctgcaggcagtttaccttttaccggaaactcaggcagtgttaca |
| | | gcgcctgggacaacaggacttgatatctattctggagggaaaacggtgatagatcccgttcttgaatatcgcctgtctcaaatcca |
| | | gagtcgcattaacgaagatcgcttcctcaaaaataacggctccggaaatgaaattggtttttggatctttgattatcccgcgcagt |
| | | gcgaactgcaggtacgggagcatttgaaatatctgctccggcatctggaaaaggaccataaatttgcctgtctgaatgtcttccaa |
| | | atcatcatcgatatgctcaatgaacgcggccttttcgagcgcgtctgccagcaggaagtcaaagtgggtactgagacgctgaaaaa |
| | | gcagcttgctggtccgttaaatcagaaaaagatcgctgattttatagcgaaaaaagtcgatctggctgcccaggattttgtcattc |
| | | ttaccggcatgggcaacgcctggccattagtacgcggtcatgaactgatgagtgccttgcaggatgtcatggggttcaccccactg |
| | | ctgatgttttatcctggcacctacagcgggtacaacctttccccgctcacagacaccggttcacaaaattattatcgcgctttcag |
| | | actggtaccagatacgggacccgcagcaacattgaatcctcaatgaagagcataacaatgaatattgaacagatttttgaaaaacc |
| | | tctaaaacgaaatataaacggggtagtcaaagcagagcaaaccgatgatgccagcgcgtacatcgagttagatgaatatgtcatca |
| | | cccgcgaactggaaaaccatcttcgccatttcttcgaatcctatgttcctgccactggcccggaacggatccgtatggaaaacaag |
| | | atcggcgtatgggtttcaggcttcttcggttcaggtaaatcgcactttattaagattctttcttatcttttatctaaccgcaaagt |
| | | tacacataacggtacggaacgtaatgcttactccttctttgaagataaaatcaaagatgcattattccttgccgatattaacaaag |
| | | cggtgcattacccgactgaagtcattctgttcaatattgattcgcgtgccaacgtagatgacaaagaagatgccattcttaaagtc |
| | | ttcctgaaagttttcaacgaacgcattggatactgcgctgattttccgcatattgcccatcttgagcgcgagctggataaacgcgg |
| | | tcagtatgaaacctttaaagccgcgtttgccgatatcaatggctcgcgctgggaagacgagcgcgacgcttactacttcatcagcg |
| | | atgacatggcacaagcattaagccaggccacgcagcagagtcttgaatcctcccgccaatgggtggaacaactcgacaaaaacttc |
| | | ccgctggatatcaataatttttgccagtgggtaaaagagtggctggatgacaatggtaagaacatcctctttatggtggatgaagt |
| | | cggtcagttcattggcaaaaatacgcaaatgatgctgaagctgcagactattactgaaaaccttggggtaatttgcggtggccgcg |
| | | catgggttatcgtgacttcgcaggccgatatcaacgcggcaatcggtggtatgagcagtcgcgacggacaggacttctccaagatc |
| | | caggggcgcttctctacacgcctgcaactttccagctctaacacatcagaagttatccagaaacgtttgttggtaaagactgacga |
| | | agcaaaagcggcactggcaaaagtgtggcaagagaaagccgatatcctgcgtaaccagctggcttttgacactacaacaactactg |
| | | cactacgtccttttaccagcgaagaagagttcgttgacaactacccgtttgtcccgtggcactatcagattctgcaaaaagtgttt |
| | | gaatctattcggacgaaaggtgcagcgggtaaacaattggccatgggtgagcgttctcagctggaggcattccagacggcggcgca |
| | | gcaaatctcagcgcaagggctggattctctggtgcctttctggcgcttctatgccgccattgagagcttcctggaacctgccgtta |
| | | gccgcaccatcactcaggcttgccagaatggcattcttgatgagttcgatggcaacctgcttaaaacgctgttcctgatccgctat |
| | | gtggaaacgctgaaaagcaccctggataacctggtcacattgtctatcgataggatcgatgccgataaagttgagttgcgccgccg |
| | | ggtcgaaaaaagtctcaacacgcttgaacgcctgatgctcattgcgcgcgttgaagataaatatgtgttcctgaccaacgaagaga |
| | | aagagatcgaaaacgagatccgtaacgttgatgtcgatttctctgcgatcaacaaaaaactggcatcgatcatctttgatgacatt |
| | | ctgaaaagccgtaaatatcgttatccggctaacaagcaagactttgatatcagccgcttcctgaacgggcatccattagacggcgc |
| | | agtgcttaacgatctggtggtgaagatcctgacccctaaagatccgacttattcgttctataacagcgatgcgacctgtcgccctt |
| | | atacgtcagaaggcgacggctgtattttgattcgtctgcccgaagagggccgtacctggagcgatattgatttagtcgtccagact |
| | | gaaaagttcctcaaagataacgccgggcaacgtccggaacaggcaaccctgctctcagaaaaagcgcgtgaaaacagcaaccggga |
| | | aaaattactccgtgttcagttggaatcactacttgcagaagcagacgtctgggcgattggcgaacgcttaccgaaaaaatcctcca |
| | | cgccatcgaacattgtcgatgaagcctgccgttacgtgattgaaaacaccttcggcaagctgaagatgctgcggccttttaacggt |
| | | gacatctcccgtgaaattcatgcattactgacggttgagaacgacaccgaactggatctcggtaacctcgaagagtccaaccccga |
| | | cgccatgcgcgaggtagaaacctggatcagcatgaatatcgaatacaataaacctgtgtatttacgcgatattctgaaccattttg |
| | | cgcgtcgcccttatggctggcccgaagacgaagtgaaactgctagtagcccgtctggcctgcaaaggtaaattcagcttcagccag |
| | | caaaacaacaacgtcgagcgaaaacaggcgtgggagttatttaataacagccgccgccatagcgaattgcgtctgcataaagttcg |
| | | ccgtcatgatgaagcgcaggtgcgtaaagccgcgcaaaccatggctgacatcgctcagcagccgtttaacgaacgggaagagccgg |
| | | cgctggttgaacatattcgtcaggtatttgaagagtggaagcaagagctgaacgtattccgcgccaaggcagagggcggaaacaat |
| | | ccggggaaaaacgagattgaatccggtctgcgcctgcttaatgccattcttaatgagaaagaagattttgccctgatcgaaaaagt |
| | | ctcatcgctgaaagatgaacttctggatttcagcgaagaccgtgaagatttggtcgacttctaccgtaagcaattcgccacctggc |
| | | aaaaactgggtgctgcgctgaatggcagctttaaatctaaccgcagcgcgctggaaaaagacgccgcagcggttaaagcgctgggc |
| | | gagctggaaagcatctggcaaatgccggaaccttataagcatctcaatcgcatcacgccgttgattgaacaggtccagaacgtcaa |
| | | ccatcagttagtcgaacagcatcgccagcacgccctcgaacgcattgacgcccgcattgaggaaagccgtcaacgcttgctggaag |
| | | cgcacgccacgtcggagctgcaaaacagcgttctgctgccgatgcaaaaagccagaaaacgcgctgaagtcagccagtcgattccg |
| | | gaaattttggcggaacagcaagagacaaaagcgctgcaaatggatgcagataaaaagattaacctgtggatcgacgagctgcgtaa |
| | | aaagcaagaagcacaactccgggcagcaaatgaagctaaacgcgctgccgactcagaacagacttatgttgtggtggaaaaaaccg |
| | | ttatccaaccggtaccgaaaaaaacgcatctggtgaatgtcgccagtgagatgcgtaatgccaccggtggtgaagttctggaaacg |
| | | accgaacaggtggaaaaggcgctcgacacgttacgcacaacgctgctggccgtcattaaagcaggcgatcgcattcgccttcagta |
| | | actcccatttcagggcagcactctgctgccctttgcaggattttctatgaataccaataacattaaaaaatatgccccacaggccc |
| | | gtaacgacttccgcgatgcggtgatccagaagctaacgacgcttgggatcgctgcagataaaaaaggcaatttgcagattgccgag |
| | | gccgaaaccattggcgagaccgtgcgttacggtcagtttgattacccgttatcgacccttccccgccgcgaacggctggtaaaacg |
| | | cgcccgtgagcagggttttgaggtgctggttgagcactgcgcctacacctggtttaaccgcttatgtgcaattcgctatatggagc |
| | | tacacggttatcttgagcacggcttccgtatgttgtcccacccggagacgccgaccgcgtttgaggtgctggatcatgtgccggaa |
| | | gtggcagaagccctgctgccggaaaataaggcgcagctggttgaaatgaagctttccggtaatcaggacgaagccctgtaccgcga |
| | | actgctgctggggcagtgccacgccctgcaccacgcgatgccgttcctgtttgaagcggtagatgacgaagcggaactgctgttgc |
| | | cggataacctgacccgtaccgactctattctgcgtgggctggttgatgatattccggaagaagactgggagcaggtagaggttatc |
| | | ggctggctgtatcagttctatatttcggaaaagaaagatgccgtgattggcaaagtggtgaagagcgaagatattcctgccgccac |
| | | ccagctgtttacgccaaactggattgtgcagtatctggtacaaaactccgttggccgccagtggttgcagacctacccggactcgc |
| | | cgctgaaagacaaaatggagtactacatcgagcctgcggaacaaacgccggaagtgcaggcgcagctggcggcgattaccccagcc |
| | | agcattgaacccgaaagtattaaagtgctcgacccagcctgcggctccggtcatattttgattgaagcctataatgtgctgaaaaa |
| | | tatctacgaagagcgtggttatcgcgggcgtgatattccacaactgattctggaaaataatatttttggtcttgatatcgacgacc |
| | | gcgcggcacagctttccggctttgcattattaatgatggcgcgtcaggatgaccgcagaatatttacccgcgatgtacgtctgaat |
| | | attgtctctttgcaggaaagcctgcatctggatatcgccaaactctggcagcaactgaatttccaccagcaggtacaaaccggcag |
| | | tatgggggatatgtttgctgaaaataacgcgttaacccaaactgacagcgcagaatatcagctgctgatgcgcacgctgaaacgct |
| | | ttgtgaatgcaaaaacgctgggctcactgattcaggtgccgcaggaagaagaagcggaactgaaggtattcctggacgcgttgtat |
| | | cgcctggaacaggaaggcgatttccagcagaagacggcggcaaaagcgtttattccgtttattcagcaggcgtggattttagcgca |
| | | gcgatatgatgcggtagtggcgaatccgccgtatatggggggtaattatatggagacagaacttaagaatttcgtctcttcttact |
| | | accctcaaggaaaggcggatctttattcttcatttatggtcagattacttttacaattaaaagataatcgcactttaagcctaatg |
| | | accccctttacttggatgaatttatcatcatttgaagagctccgaaaaattatacttacaaatttcagcattcagtcattagtaca |
| | | gcctgaatatcattcattttttgagtcagcttatgtcccaatttgtgcttttagcatttcaaataccccattaagctggaatgcaa |
| | | aattttttgatttatcagatttttatggagaaaaaaatcaagctccaaattttcagtatgcaattaaaaatgacaataaatgtcat |
| | | tggaaatataacagaatcaccacggactttctatgtactcccggatatatcattgcttactctctgcctgattctgcgttatcttg |
| | | cttcaaaacatccaaaaaacttcatgatgtttgcaatctaaaacaaggattaattactggtgataatgaaagatacctaagattct |
| | | ggcatgaaatcagctataactctttcagtctcaatgaaaaaagaaaaaaaacaaaatggttcccatatcaaaaaggtggtgcatac |
| | | cgtaaatggtatggtaataatgattatgttgttgactgggagaatgatggttattccattaaaaacttttataatgacaaaggtaa |
| | | attacgctcacgccctcaaaacatacaattttattgtaaagagggtttaacatggacaagtttaactatttcgtcactatcgatga |
| | | gatatgtaccaaatggatatatttttgatgcaaaaggacctatgtgttttccgaaatcctctttggatatctggaatattcttggc |
| | | tatgcgaatagcaaagtaatagatatatttctcaaacaattagcgcccaccatggattattctcaagggcctgttggaaatgtccc |
| | | attcaaatttaacgatggtgatttgaacgagataataaaagaactcgtaaacattcacaaacgtgactgggatgaaaatgaaacat |
| | | cttttgagtttaagagagatatgttggttcatttttcaagagatattaacactattaagggtagttttacactaaggcaaggggaa |
| | | aataaaaaagcgattaacagaacaaaatttttagaagaaatgaataactctttctttataaattgctttaatctaactgatatttt |
| | | atctccagaaattgaactaaacaaaatcacgttaacgcatgcaactattgaaattgatattcaaaaaataatttcatatgcaatag |
| | | gctgccaaatgggacgttactcccttgatcgcgaaggtctggtatacgctcatgaaggcaataatggcttcgccgatcttgtcgcc |
| | | gaaggtgcttataaaagcttcccggctgatagtgacggcattctgccgctaatggatgaagagtggtttgacgatgacgtcacctc |
| | | tcgcgtcaaggagtttatccgcaccgtttggggcgaagaatatttgcgcgaaaacctcgattttatagccgaagttctcaagccca |
| | | aaaaaggcgaatctgcgctggagaccattcgtcgctatctttccacccagttctggaaagatcatctgaaaatgtataaaaagcgt |
| | | ccaatctactggctattcagctccggtaaagagaaagcgtttgagtgcttggtgtatctgcatcgctataacgatgccacgctgtc |
| | | gagaatgcgtaccgaatatgtggtgccgctgctggcgcgttatcaggccaatattgatcgcctgaacgatcaacttgatgaggctt |
| | | ctggcggtgaatccacacgtctgaaacgcgaacgcgacagcctgatcaaaaaattcagcgaactgcgcagctatgacgatcgcctg |
| | | cgtcactatgctgatatgagaatcagtattgatctcgacgatggcgttaaggttaactacggcaagtttggcgatctgctggcaga |
| | | tgtcaaagccatcaccggcaatgccccagaggtgatctaaaccagacggcacgttctcctgttgccgggttctgcccggtggcaaa |
| | | taccaccgggaaacgcgccgctgctgacatttctccacctcacttcatgataaaatgcgccaccgtgtcaaaatctccttttcgcg |
| | | ttttggcgctttcttattcatcgtaacaacatgggattgtgaacttgcaaaatcaggactttattgctggccttaaagctaaattt |
| | | gccgaacatcgcatcgttttctggcacgatcccgataaacgttttattgaggaactggaacagctcaagcttgaaagcgtcacgct |
| | | aatcaacatgacccacgagtcacagctggcggtaaaaaaacgcatcgagattgatgagccagaacagcagttcctgctgtggttcc |
| | | cccatgatgcgccgcctcatgaacaagactggctgctggatatccgcctttacagcagcgaattccatgccgattttgccgccatc |
| | | accctgaacacgctgggcattccccagcttggcctgcgcgagcatattcagcgacgcaaggccttcttcagcactaaacgcacgca |
| | | ggcgctgaaaaatctggcgacagaacaggaagatgaagcctcgctggataagaaaatgattgcggtgatcgctggcgcaaagaccg |
| | | cgaaaaccgaagacattttgttcaacctgattacccagtacgttaaccaacaaatagaagacgacagcgaactggaaaacacgcag |
| | | gcgatgctgaaacgccacggtctggactcggtattgtgggaaatgctcaaccacgaaatgggctaccaggcagaggagccatcgct |
| | | ggaaaacctgctcctgaaactgwtgtaccgatctctctgcccaggccgacccacagcagcgcgcctggctggaaaaaaatgtcctg |
| | | ctgacgccatccggcagagcatctgccctggcatttatggtgacctggcgtgccgatcgtcgctataaagaggcttatgactactg |
| | | cgctcagcaaatgcaggccgccctgcacccggaagatcattaccgactcagctcgccgtatgatttgcacgaatgcgaaaccaccc |
| | | tcagcatcgaacaaaccattattcatgcgctggtaacacagctgctggaagagagcaccacgctcgatcgggaagcctttaaaaaa |
| | | ctgctctctgagcgccagagcaaatactggtgtcagacacaaccagagtattacgccatctatgacgcattgcgccaggctgagcg |
| | | gttgctgaacctgcgcaatcgccacatcgatggtttccactaccaggacagcgccaccttctggaaagcctactgcgaagaactgt |
| | | tccgcttcgaccaggcttatcgcctgtttaatgaatatgccttgctggttcacagcaaaggagcgatgatcctcaagagcctggat |
| | | gattatatcgaggcgctctacagcaactggtatctggcagagttaagccgtaactggaacgaagtgctggaagcggaaaatcgtat |
| | | gcaggcgtggcaaatccctggcgtgccgcgtcagcagaacttcttcaatgaggtggtgaagccacagttccaaaatccgcaaatca |
| | | aacgcgtgttcgtgataatttccgatgccctgcgttatgaagtggcggaggagctggggaatcaaatcaataccgagaaacgcttt |
| | | accgcagaactgcgctcgcagctcggcgtgctccccagctacacccaactgggaatggcggcattgctgccccatgaacaactttg |
| | | ctatcaacccggtaacggcgacatcgtttatgctgatgggctgtcgacctcgggtattcctaaccgcgataccattctgaagaact |
| | | ataagggaatggcgataaaatcgaaggaccttctggagttaaaaaatcaggaagggcgagaccttattcgcgattacgaagtggtg |
| | | tatatctggcataacacgattgatgccactggcgacacggcatccacggaagataaaaccttcgaagcgtgccgcacggcggtggc |
| | | tgaactgaaagatttagtcaccaaggtgatcaaccgcctccacggcacacgcatttttgttacggcggatcacggtttcctgttcc |
| | | agcaacaggcgctttcggttcaggataaaaccactctgcaaattaagccggaaaacaccatcaagaaccacaaacgctttattatc |
| | | ggccatcagcttcccgccgatgatttttgctggaaagggaaagtggcggataccgcaggcgtgagcgacaacagcgagttcctgat |
| | | tccgaaagggatccagcgcttccatttctctggcggcgcgcgcttcgttcatggcggcaccatgttgcaggaggtttgcgttccgg |
| | | tattgcagataaaagccctgcaaaaaaccgccgcagaaaaacagccacagcgccgcccggtggatattgtcgcttaccatccgatg |
| | | attaagctagtgaacaatatcgataaagtgagcctgttgcagacgcatccggtgggcgaactttatgaaccgcgtatcctgaacat |
| | | ttacattgtcgacaacgccaacaatgtggtctcgggcaaagagcgcatcagctttgacagtgataacaacaccatggaaaaacgcg |
| | | tacgcgaagttacgctgaagctgattggcgctaacttcaaccgtcgcaatgagtactggttgatactggaagacgcacaaacggaa |
| | | acggggtatcagaagtacccggtcattatcgatctggcgttccaggatgatttcttctaagtgaggcgatatgcaaacccatcatg |
| | | atttacctgtttcaggcgtatccgcaggggaaattgcctccgagggttacgatctggacgccctgctgaaccagcattttgctggt |
| | | cgcgtggtgcgtaaagatctcaccaagcaactcaaggaaggggcaaacgtcccggtgtatgtgcttgagtatctgctcggcatgta |
| | | ctgcgcctctgacgatgacgatgtggtcgagcaagggttgcaaaacgttaagcgtattctggctgataactatgtgcgcccggatg |
| | | aagcggagaaagtgaagtcgctgatccgcgagcgtggttcgtacaaaatcatcgataaagtgtcggtgaaactgaaccagaaaaaa |
| | | gacgtttacgaagcccagctttctaacctcggcatcaaagacgcgctggtgccctcgcagatggttaaagacaacgagaagctact |
| | | gacgggcggtatctggtgcatgattaccgtcaactatttctttgaagaagggcagaagacctcacccttctcattgatgacgctca |
| | | agcctatccagatgccgaatatggatatggaagaggtgttcgatgcgcgtaaacactttaaccgtgaccagtggatcgatgtgctg |
| | | ctgcgctcggtgggtatggagcccgccaatattgagcaacgcaccaaatggcaccttatcacccgtatgatcccgttcgtggagaa |
| | | caactataacgtttgcgagctggggccgcgtggcaccggtaaaagccatgtgtataaagagtgttctcctaactccctgttagttt |
| | | ccggcgggcaaacgaccgttgccaacttgttctacaacatggccagtcgccagatcggcctggttggcatgtgggatgtggtagcg |
| | | ttcgacgaagtcgcggggatcactttcaaagataaagacggcgtgcaaatcatgaaagattacatggcgtcaggatctttctctcg |
| | | cggcagagattcgattgaaggtaaagcgtcgatggttttcgtcggcaacatcaatcaaagcgtagagactctcgttaaaaccagcc |
| | | atttgctggcaccatttccgactgcgatgattgatacagcatttttcgaccgctttcatgcctatattcccggttgggaaatcccc |
| | | aaaatgcgcccggaattctttaccaaccgttacgggctgattacggattatctcgctgaatatatgcgcgaaatgcgcaaacgcag |
| | | tttctctgatgcgattgataaattctttaagctgggtaacaacctcaaccagcgtgacgttattgccgttcgacgtaccgtgtcgg |
| | | ggttgttaaaactcatgcatcccgatggcgcgtacagcaaagaagatgtgcgagtctgcctgacctatgcgatggaagttcgccgc |
| | | cgcgtgaaagagcaacttaaaaaactgggcggtctggagttcttcgatgtgaactttagctacatcgacaacgaaacgctggaaga |
| | | gttttttgtgagcgtaccggaacagggcggcagcgaacttattcctgccggaatgccaaagccgggtgttgtgcatctggtcactc |
| | | aggcagaaagcggcatgaccgggctgtatcgttttgaaacacagatgactgccggtaatggtaagcatagtgtatcgggtctgggt |
| | | tcaaatacctccgcgaaagaagctatccgcgtcggtttcgattacttcaaaggcaatttgaatcgggtaagcgcggccgcgaaatt |
| | | ctccgatcatgaatatcaccttcatgtcgttgaactgcataatactggcccaagcaccgcaaccagtcttgctgcgcttatcgctt |
| | | tatgttcgatattgctggcaaaaccggtgcaggaacagatggtggtgttgggcagtatgacgcttggtggggtaattaacccggtg |
| | | caggatcttgccgccagtttgcagctcgccttcgacagcggtgcaaaacgggttctgttgccgatgtcctcggctatggatattcc |
| | | aacggttccggcagagttatttaccaagtttcaggtgagtttttactcagacccggttgatgctgtttataaggcgctgggtgtga |
| | | attaacgtagtaactattttaatgaac (SEQ ID NO: 3) |
| 2 | pLG004 | ggtgaacgtttggttgatagggtagtaaaactagtaatcatcctataattagctatattcgtggttattagattgaaaacagataa |
| | | cattaacaaaatctataaatcgatttgaatgatttttttcatcaatactgttgtaagctcctgctatcaaaagttttgcacacaat |
| | | ctataagctcccagaattgcttgtataaatgctatcattggcgctgtcccgatcgagggagcaaggaggggactctcttgtgccat |
| | | gcgattaatcactggggctctaagtgaaatttagtgggactaaatactaattggaacgtgagataaaaatgcacaaatatccctct |
| | | ataatagttaatatcaaccttcgagaagccaaactgaaaaagaaggtacgtgagcatttacaatccttgggttttacaagatctga |
| | | ttctggagcgctccaggccccgggaaataccaaagatgtaatacgggctcttcatagttctcaacgagctgagcggatatttgcaa |
| | | accaaaagttcataacgctaagagcggcaaagcttattaaatttttcgcatccggcaatgaggtcattccggataagatttcaccg |
| | | gtacttgaacgtgtaaagtcaggaacctggcaaggagatctctttaggttagcagcattaacttggtccgtacctgtttcaagcgg |
| | | atttggaaggcgtctccggtatcttgtatgggatgaaagcaacggaaaattgatagggctgatcgcaattggtgaccctgtgttca |
| | | accttgcagtccgagataatttgattgggtgggatactcatgccagaagttcccggcttgttaatttgatggatgcatacgtcctc |
| | | ggtgctcttcccccttataatgccctgctgggaggaaaattaattgcatgtctgcttcgtagccgcgatctttatgatgactttgc |
| | | aaaggtctatggtgataccgttggagtaatatctcaaaaaaagaaacaagcacgtcttttggctattacaacaacatcgtctatgg |
| | | ggcgctcatcggtatataaccgtttaaagctggatggaattcaatatttaaaatcgattggatatacaggcggttgggggcatttt |
| | | catatacctgatagcttgttcattgaattacgtgattacttacgtgatatggatcacgcttatgcagatcattatatgtttggtaa |
| | | tgggcctaactggcgtttacgtacaactaaggcagctttaaatgcactaggatttagagataatttgatgaagcatggaattcaac |
| | | gtgaagtgtttatcagtcagctagcagaaaatgcaactagtattctgcaaacaggcaaaggtgaaccagatctaacctctttgctt |
| | | tctgctaaagagatagctgagtgtgcgatggcacgatggatggttccacgatcaattcgcaatccagaatatcggctttggaaagc |
| | | aagagatctatttgattttattagtaatgactcgctaaactttcccccgtttgacgagatagcgaaaacagttgtctaatcttaac |
| | | tgaagggggagtaagtgaattacgctattgataagttcaccgggacactgatattagcagctcgagcaacgaaatatgctcaatat |
| | | gtttgcccagtttgtaaaaaaggtgttaacctccgtaaagggaaggttatacccccatattttgctcatttgcccggacatggtac |
| | | gtcagactgtgaaaattttgttcccggaaattctatcattgtcgaaactattaaaactatttcaaagcgatatatggatttgcgct |
| | | tattgattcctgtcggaagtaatagtcgagagtggtcattagaattagtgttgccaacctgtaatttatgtagagcaaagataacg |
| | | ttagatgtaggaggcagaagccaaacgcttgatatgaggagtatggtaaagagtcgccagattggtgctgaattatcagtaaaatc |
| | | ttaccgtattgtttcatatagtggtgaaccagatccaaaatttgtaacagaagttgaaagagaatgcccaggtttaccttctgagg |
| | | gagcagcagttttcactgctttagggcgtggggcatcgaagggatttccacgagcacaagagttaagatgtactgaaacatttgcc |
| | | tttctttggcgacaccctgttgctccagattttcctgatgaattagaaataaaaagtttagctagtaaacagggatggaatttagc |
| | | tcttgttacaattcctgaagtcccttctgtggagagtatttcatggctaaaatctttacataccttcctgttgttcctgccagaac |
| | | atctattacagcaatttggccgttcctaaatcaaaaaacaagtattaatcatgtcgaatgtgtttattctgacacaatattgttgt |
| | | caacaaatatggcaccaacatcatcagaaaatgttggaccaactatgtacgcacaaggttcctctttattactttcagcggttggt |
| | | gttgaaacatcacctgctttcttcattctaaatcctggagaaaatgactttgtgggcgtttctggctcaattgagcaggacgtaaa |
| | | cttatttttttctttctataaaaaaaacgtttctgtacccagaaaatatccctcaatagatttggtttttactaagaggaataaag |
| | | aaaagaccatcgtttccttacatcaaagaagatgcattgaagttatgatggaagcacgaatgtttggccataaattagaatacatg |
| | | tctatgccttctggtgttgaaggagtggcaagaattcaaagacaaactgaaagtaatgttattaagttagtttctaatgatgacat |
| | | tgcagctcatgataagagcatgcggttactatctcctgttgcgttatctcaattatctgattgcttagcaaacttaacatgtcatg |
| | | tagaaatagattttttaggtcttggtaaaatatttttacctggttcttctatgctatcattagatgacgggaaatttattgaatta |
| | | tctcctaatcttcgctcacggatattaagttttatacttcaaatggggcacaccctccatggttttagtttaaataatgatttttt |
| | | attagttgagaaattagtggatttgcagccggaaccacacttattaccgcattatagagcattggtaaaagaagttaagaccaatg |
| | | gatttgaatgtaaccgctttagataaggtgccttcgaatgagttaccaatatagccaagaggcaaaggaacggatctctaagttgg |
| | | gacaatccgaaattgttaactttatcaatgagatttctccaactttacgacgtaaagcttttggttgtttaccaaaagtaccggga |
| | | ttcagggcaggacatcccactgaaattaaagaaaaacagaaaagattgattgggtatatgttccagtcacatccttcctctgagga |
| | | gagaaaagcatggaaaagtttttctcttttttggcagttttgggctgaagagaaaattgacaaatcatttagtatgattgataatt |
| | | taggattaaaagaaaactctggctctatttttattagagagcttgctaaaaactttcctaaagttgctagagagaatatcgagcgc |
| | | ctgtttatctttagtgggtttgctgatgatccagacgttataaatgcatttaacctttttcctcctgcagttgttcttgcccgcga |
| | | tatcgtgattgatactcttccaattcgtttagatgagcttgaagcacgtattagtttaattgccgataatgttgagaaaaaaaata |
| | | atcatattaaagaacttgagttaaaaatagatgctttttccgaacagtttgataattactttaataatgaaaagagcagtttaaaa |
| | | ataattaatgaactacaatctttgataaactcagagactaaacaatctgatattgctaataaagctattgacgagctttatcattt |
| | | taatgaaaaaaacaaacagctaatattatctcttcaagaaaaattagattttaatgctctggctatgaatgatatttctgagcatg |
| | | aaaaattgataaaaagtatggctaatgacatttcagaatttaaaaatgcattaacgatcttgtgtgataataaaataaagaataac |
| | | gagttagattatgtcaatgaattaaaaaaactcactgaacgaatagatacacttgaaataaacacatctcaagctagcgaagtgag |
| | | tgtcaccaatagatttacaaaattccatgaaatagcgcactatgaaaattatgagtatctttcatcctccgaagacatatctaata |
| | | gaatttctttaaatttacaggctgttggattgacaaaaaattcagcagaaaaattggctagattgacattagctaccttcgtttct |
| | | ggacaaatcattcaattcagtggctctttggcagatattatcgcggatgcaattgccattgctattggtgcaccacgttatcacat |
| | | atggagagttccagttggtattatttctgacatggatgcttttgattttatagagactatagctgaatcatctcgctgtctccttt |
| | | tgaaaggggccaatctttcagcatttgagatttatggagcggcaattagagatatagttgttcaacggcaaatacatccaacaaat |
| | | tatgaccatctggcattgatagctacctggaaacaaggcccagctacattccctgatggaggaatgttggccgagttgggacctgt |
| | | tattgatactgatacattaaaaatgcgtggtttatcagctactttaccccaattgaaaccaggttgtcttgccaaggataaatgga |
| | | caaatattgatggactacatcttgatagtgttgatgattatgtagatgaattaagagcattactggacgaagctggatttgatggg |
| | | ggaactttgtggaagagaatgattcatattttctatacttcactcataaggatccctaatggaaattatatttatgatctttattc |
| | | tgtcttgtctttttatactcttacatgggcaaaaattaaaggtggccccgtccaaaagatagaagatattgccaatcgtgaattaa |
| | | aaaattatagtgcaaaaatatcttcttgaggaggtggttaatggagtggagagcagtatcacgagacaaagcactggatatgttat |
| | | caactgcattaaattgtcgatttgatgatgaagggttgagaatttcagcagtttcagaatgcttaaggagcgtattatatcaatat |
| | | tctatatctgaaacagaagaagctaggcaaactgtaacctcgcttcgactcactagtgcagtaaggcgaaaattggtacctttatg |
| | | gccagacattgctgatattgataatgctatacatccgggcattatgtctatattgaacagcttggctgaattgggtgacatgatta |
| | | agttagaaggtggtaattggctaacagctcccccacatgcagtacgaattgacaataagatggctgttttttttggtggagagcct |
| | | tcctgtacattttcaacgggcgtggtagctaaatctgctggaagagttcgcttggttgaagaaaaagtgtgtactggaagtgttga |
| | | aatctgggatgcaaatgagtggattggtgccccagcagaaggcaatgaagaatggtcatccagactactatctggaactatttccg |
| | | gctttatcgatgcacctggcaatatgagtgaaacgactgcatatgtgcggggaaaatggctccatttgtcagaactttcttttaat |
| | | aaaaagcaaatctacttatgcagaatgtccgttgataatcacttttcctattatttaggagaaattgaagctggacgcttatgtag |
| | | aatgaattcgttagaatcgtctgatgatgtcagaagattacgtttttttctcgatacaaaagataattgtccgctaaaggtccgta |
| | | tcaaaatatctaatgggctagcaagattaagattaaccagaagattaccaagacgagaaacgaaggtactcctgctaggctggaga |
| | | gaatcaggttttgaaaatgaacattcaggaataacacaccatgtattccccgaggaaatattacccatagtgcgtagcgcttttga |
| | | agggcttggtattatttggattaacgaattcacgcgacggaatgaaatatgattaataaaaataaagtaactgaacgttcaggtat |
| | | acatgataccgtgaaaagccttagtgaaaatctgagaaaatacattgaggcacaatatcatatccgggatgaagggttaattgctg |
| | | agcgacgagcgcttttacagcaaaatgaaactattgctcaagctccttatatagaagcaaccccaatttatgaacctggtgcgcca |
| | | tacagtgaattgcctattcccgaagcagcaagtaatgtgctaactcaactatcagaacttggaattggcctctatcaacgccccta |
| | | taaacaccaatcacaggcacttgagtcatttcttggcgaaaacgcttctgatctggtcattgcaacaggtacaggctccggtaaga |
| | | ctgaaagctttctaatgccaattattggaaaattggcgattgaatcttccgagagacctaaatctgcatcccttccaggttgtaga |
| | | gcaattttattatatccaatgaatgcattagttaacgatcaacttgctcgtatcagacgtctttttggtgattctgaagcctctaa |
| | | aatactgagatctggaagatgtgcccctgtacgctttggcgcttatacgggaagaacgccttaccctggtcgtcgtagctctagac |
| | | gagacgagctttttatcaaaccccttttcgatgagttttacaataaactcgcaaataacgcccccgtacgtgcggaactgaaccgc |
| | | attggtcgctggccaagtaaagatcttgatgctttttatgggcaaagcgcatctcaggctaaaacctacgtctcaggcaaaaaaac |
| | | gggtaagcaatttgttttgaacaattggggggagaggctaattacccagcctgaggatcgtgagctaatgacccggcatgaaatac |
| | | agaatcgctgtccagaattactgataacgaactactccatgcttgagtatatgctgatgcgacctatcgagcgtaatatttttgag |
| | | cagactaaggaatggctcaaagctgatgagatgaatgagcttatcttagtgcttgatgaagcgcatatgtatagaggagcaggggg |
| | | agcagaggtagcccttttaatacgtcgcctctgtgctcggttggatattccccgggaacgtatgcgctgcatccttaccagtgcta |
| | | gtctagggtccattgaggatggagaacgttttgcccaagacttaactggcttatcaccaacctcttcgaggaaatttcgaattatt |
| | | gagggtacaagggaatcgcgtcctgagtcacaaattgttaccagtaaagaagctaatgcactggctgaattcgacctaaattcatt |
| | | tcagtgcgtagctgaggatcttgaatctgcatatgcagcaatagagtctcttgccgaacgaatgggctggcaaaagccgatgataa |
| | | aagatcatagtacactacgtaattggttatttgataatttgactggttttggtcctattgaaacgcttattgaaatagtttcaggt |
| | | aaagcggttaagctaaatatcttgagtgaaaacctttttccagactctccacagcaaatcgcagagcgagcaacagatgcattact |
| | | cgcattgggttgctatgctcagagggcatccgatggcagagtgcttattccaactcgcatgcatcttttttatcggggattaccag |
| | | gtctttatgcctgtatagatcccgattgtaatcaacgtttgggtaaccatagcgggccaactatacttggccgcctttatacgaaa |
| | | ccactggatcaatgtaaatgcgcttcaaaagggcgagtctacgaattatttacccaccgtgactgcggtgcggcttttattcgtgg |
| | | atacgttagttccgaaatggactttgtatggcaccagccgaacggaccattatcagaagatgaggatatcgatcttgttcccatag |
| | | atatattggtcgaggaaacacctcatgtacatagtgattaccaggacagatggctacatatagcaacaggacgcctttctaaacag |
| | | tgtcaagatgaggattctggttatcgtaaagtctttatacctgaccgagttaagtctggatcagaaattacatttgatgaatgccc |
| | | tgtttgtatgcgtaagacaagaagtgctcagaatgaaccgtctaaaattatggatcatgttacaaaaggggaagcaccttttacaa |
| | | cgttagtacgtacacagatatctcaccagccagcgagtcgtcctattgatggtaaacatcccaatgggggaaaaaaagtacttatt |
| | | ttttctgatggccgacaaaaagcagctcggcttgcacgtgatattcctagagatattgagcttgatttgtttcggcaatccattgc |
| | | tctcgcctgttctaaactgaaagatatcaatcgggaacccaaaccaacatcagtactttaccttgctttcctatcagtcctttctg |
| | | aacatgacttgcttatttttgatggggaagattcacgaaaagttgtaatggcccgtgatgaattttatcgtgattataatagcgat |
| | | ctggctcaagcttttgatgatagcttcagcccccaagagtcaccgtcacgatataaaatagcgttgcttaaacttttatgtagcaa |
| | | ttactattctctttccggaacaacagttggttttgttgaaccatcgcagcttaaatcaaaaaaaatgtgggaagatgtgcagtcca |
| | | agaagctcaatattgagagcaaggatgttcatgctttagctgttgcttggattgataccttactcactgaatttgcttttgatgaa |
| | | tctattgattcgacactacgaatcaaagcagctggattctacaaacccacttggggtagtcaaggacggtttggaaaagctcttag |
| | | gaaaaccctgatacagtatcctgctatgggggagctttatgtggaagttttggaggagatttttcgtactcatctgacattaggaa |
| | | aagatggtgtctactttcttgctccaaatgcactacgtctgaaaatagatctcttgcatgtctggaaacaatgtaatgactgcacg |
| | | gcactaatgccatttgctttagaacattctacttgccttgcttgtggtagtaacagtgtcaaaacagtcgagccgtcggaaagcag |
| | | ctatattaatgcacgaaaaggattctggcgttcgccggtagaagaagttttggtttcaaattcgcggcttctaaaccttagcgttg |
| | | aagagcatactgctcaactctcacatagagatagggccagcgttcatgccactacagaactctacgaactgagattccaagatgtt |
| | | cttattaatgataacgacaagcccattgatgtacttagttgtacgacgacgatggaagtgggggttgatattggatctctggttgc |
| | | tgttgctttaagaaacgtccctccgcaacgagaaaattatcagcaacgtgctgggcgagcaggccgccgtggcgcatctgtttcaa |
| | | cggtggttacatattctcaaaatggccctcatgatagttattatttccttaatcctgaacgcattgttgcaggttctcctcgtaca |
| | | cctgaagtgaaagtaaataatcccaaaatagccagaagacacgttcattcttttttagttcagaccttttttcacgagttaatgga |
| | | acaaggaatttataatcccgcagagaaaactgccatacttgagaaagcacttggtactacacgagatttttttcatggagcaaaag |
| | | atactggcctaaatctcgatagctttaataattgggttaaaaaccgtattctatctactaatggtgatttgagaacaagtgttgca |
| | | gcatggcttcctcctgttcttgaaactggagggctttctgccagtgactggtttgctaaggtagcagaggaatttttaaatacact |
| | | ccatgggctggctgaaattgttccacaaactgccgttcttgttgatgaggaaaatgaagatgatgagcagacttctggtggaatga |
| | | aatttgcacaagaagaattacttgagttcctgttttaccatggtttattaccaagttatgcatttcctacaagcctctgtagtttc |
| | | ttggtagaaaaaattgtaaagaatattagaggttcttttgaggtgcgaacagtacaacagcctcagcaatcaatttctcaggctct |
| | | gagtgaatatgccccgggacgtttgattgttattgataggaaaacctatcgctctggtggtgttttttctaatgcattgaaaggcg |
| | | aactaaaccgggcaagaaagcttttcaataatcccaaaaagtttattcattgcgataagtgctcttttgtccgcgatcctcataat |
| | | aatcagaatagcgaaaatacttgtccgatctgtggtggcattctaaaagtagaaataatgattcagcccgaagtctttggacctga |
| | | aaatgccaaggaacttaatgaggacgacagagagcaagaaatcacctatgtaacagcggcacaatatccacaacctgttgatcctg |
| | | aagattttaagttcaataatggaggtgctcatattgtttttactcacgcaatagatcagaaactggtgacggtgaaccgagggaaa |
| | | aatgagggggagtccagtggtttttcagtatgttgcgaatgtggtgcggcctccgtttatgattcctactcaccggcaaagggggc |
| | | acatgaaagaccgtataaatatatagcaactaaggaaacgcctcgcttatgctctggcgagtataaacgcgtttttctcggacatg |
| | | atttccgtactgatttgcttttattacgaataaccgttgggtctccgcttgtaactgatacttcaaatgctatcgttttacggatg |
| | | tatgaagatgcattatatacaatagcggaagcactaaggcttgcagctagtcgccataaacaactggatcttgatcctgctgagtt |
| | | tggctctggtttcagaattttacccactatagaggaagatactcaggcattggatctcttcctttatgatactttatccggcggtg |
| | | cgggttatgcggaagtagcagcagcgaatctagatgacattcttactgcaacactcgcattgttagaaagctgtgagtgcgatacc |
| | | tcctgtacagattgtctcaatcatttccacaaccagcatatacaaagccgtctcgataggaaactaggtgcatctttacttcgtta |
| | | tgcactatacggaatggttcctcgttgtgcttcacctgatattcaggtagaaaaattgtctcaattgagggcaagtctggaattgg |
| | | atggttttcaatgcataattaagggaactcaggaggcacctatgattgtgagtttgaatgaccgttctattgcagtgggaagttat |
| | | cctggtcttattgatcgacccgactttcaacacgacgtatataagtcaaagcatactaatgctcatatagcctttaatgaatatct |
| | | tcttcgttcaaatctgccacaatcgcatcaaaatattagaaaaatgttgcgctgatagcagcagtattgagtgccctaaagccctg |
| | | tagggcactcaaggttttcagtgcgtgagcgggctttaactgaagccataaatgtacgtatgggagaaaatgtgaccatttaactc |
| | | gccagcaactattgcacaatgtaaaattatgcccattgag (SEQ ID NO: 4) |
| 3 | pLG005 | acggtaatgctgagtttctccattaccattgcaaatgactcaccagagcagactgaacagcgcagaagtgggattgtggatacgtg |
| | | aagtgagagtaaggggaaaatccacaataatcatctatcgaacagggaggcgaactttacacgatggttttccgggagtgcttacc |
| | | cggggttcctcacctctggctaatctctggattgagtcgcgatactccaacaaaagcaacaagctaacgcagcaagaagttaacgc |
| | | tcatcgagagtaaaatgcacacttttatggcttactcgttacaataacagccagtttgttcagaaaaccggattcagtatggccag |
| | | aataccaaccaaaaaagctaaagcaaaaaaagggtttgaagaaacattatgggatgccgcaaatcagcttcgcggcagcgttgagt |
| | | cctccgaatacaagcacgtggtgttgagcctcgtgttcctgaaattcatcagcgataagtttgaaacacgccgcaaaaaaatgatt |
| | | gccgatgggcaggcagatttccttgagatggaagtgttctaccagcaggacaacattttctacctgccggaagaggcgcgttggtc |
| | | atttatcaaacaaaatgcaaaacaggacgatattgcggttcgtattgacaccgccctctcgaccattgagaaacgtaacccaaccc |
| | | tgaaaggtgcgctgccagacaactacttcagccgtcagaatctggaaaccaaaaaactggcatcactgattgataccatcgacaac |
| | | atcgaaacgctggcacacgagactgacgttgaaacgttatcgaaagaagacctggtcggacgcgtttatgaatacttcctcggtaa |
| | | gtttgccgccactgaaggcaaaggcggtggtgagttctacacgccaaaatgtgtggtcacgctgttaactgaaatgctcgaaccct |
| | | tccagggcaaaatttatgacccgtgctgcggctcggcaggaatgttcgtgcagtcggtgaagtttgtcgagagccatcagggtaaa |
| | | agccgtgatatcgcgcgtatggtcaggagctgacagccacgacgtataaactggcaaaaatgaacctcgctattcgcggtctttca |
| | | gctaacctcggcgaacgcccggcaaacactttctttagcgaccagcacccggacctgaaagctgactatattctggcgaacccgcc |
| | | gttcaacctgaaagactggcgtaacgaagcagaattaaccaaagatccacgttttgccggttatcgtatgccgccaaccggtaacg |
| | | ccaactacggctggattttgcatatgctctccaagctgtcggctaacggcacagcgggttttgtgctggcaaacggttcgatgagt |
| | | tctaacaccagcggtgaaggcgagatccgtgcacagatgatcgaaaatgatctgatcgactgcatgattgctctgccaggtcagtt |
| | | gttttacaccacgcagatcccggtgtgtttatggtttatgaccaaatcgaaggctgccgatccggccaaaggttatcgtgatcgtc |
| | | agggcgagacgctgtttattgatgcgcgtaacctcggcaccatgattagccgcacaactaaagagttaacagcggaagatattgcc |
| | | acaatcgccgatacttaccatgcttggcgtagcacgccagaagaactggctgcacggattgcgcgtggtgacagcaagctggaaaa |
| | | atatgaagaccaggcaggcttctgcaaagttgcgaccctgcaagatattaaagataacgactacgttctgacaccgggccgctatg |
| | | tgggtgcagccgagcaggaagaagacggcgtggcatttgagaccaaaatgcgtgaattgtcgaagacgttgtttgagcagatgaag |
| | | caggcggaagaactggatcgtgcgattcgccagaatctggaggcgctgggttatggggagtaaatgggagaaaataaaacttaaag |
| | | aagttgtagatattatcactactaaagttgatgtatcgcaaattagtctttgcgattacatatcaactgaaaatatgcttaccaat |
| | | tttggaggtatatcaatagcaaatagtaaacctagcacagggaaaataacaaaatttcattctggagatattttattctcgaatat |
| | | cagaacatattttaaaaaactatggcttgcagatcgaactggtggctgttctaacgatgtaattgtattccgtcccaaaaaacata |
| | | ttaattctaattatattttatcagtattaatggatcaaaaattcatcgaatatactgttttaacatccaaaggcaccaaaatgcca |
| | | aggggtgataaaacagctatattagattatgaatttaatcttgcaccagataaatattgccaacatatcgcaaaaacaaacactct |
| | | tatatttagtaagttaaaatccaatgaagtaataaataagtcattagaacaaatgtcccaaactctcttcaaatcctggtttgtgg |
| | | attttgatccggtgatttataacgctctggatgcaggaaatccaatcccggaagctctgcaatctcgtgccgaattacgtcaaaaa |
| | | gtacgtaatagtacagattttaaaccgcttccggcggaaatccgttcgcttttcccaagtgaatttgaagaaacggagttgggttg |
| | | ggtgccgaaaggatggagtattgttcgaactgaagatattgcattgaaaataggaatgggaccatttggttccaatattaaagtat |
| | | ccacatttgttaatgctggtgtaccaattataagcggccaacatctgaaagccctccttcttatcgatggggataataatttcatt |
| | | actccagagcatgctgaaaagctcaaaaactctgctgtatatagaaaagacataatttttacacatgcaggtaatattggccaagt |
| | | ttctttaattcctgaagattctgaatatgacagatatataatttcccaacgtcaatttttcttacgcgtaaatgaatcaaaatcat |
| | | cgccgtactatttgattcattattttaggtcagaaaaaggacaacatgctctgctttctaacgcctctcaggttggtgttccttca |
| | | attgctcagccttcaacacatttgaaaaatatatcattcctaaatcccccaatggttttgcttaaagagtttgaaaaatttagcac |
| | | ccctttattccatcgctttagtaaaaatagaaaatgtggagtctcactaacagccctccgcaacaccctgctcccgaaacttatct |
| | | ccggtgagctatccctggaagatcttccggatctcagcaccgatacagaagccgcataacgcattttgcccctgtaaaatcagggg |
| | | ctttctggtaaggttttctactgatacaggaatgcttaccagaaattagccagggttggagcgcgatatgagtctctctttcagtg |
| | | aagcaaaattagaacaagcgatcattgaactgttacaggatcaggggtatcaacatctgatcggcgataatgtcccacgttcgagt |
| | | ctcgatcaggtcattatcgaagacgatctccgtcattatttagcggcacgctaccagcctgatggcattactgaagaagagattca |
| | | gcgactgatcaaacagttcaccacgcttccggcttccgatctttatgaaagcaacaaaacattttgtcgctggctggcaaatggttt |
| | | tctgttcaaacgcgacgatcggcaacaaaaagatctctacattgaattgctcgacacccggcatctacctgccgcactgcgccaga |
| | | tatttgacgccgaagatgtcctgttgcaacaggctgcggaactcccgccctcctatattaatccgccgcttaacctgattaagatt |
| | | gttaatcagcttaaaatctccggcaaagataatcagagtcgtattcctgacggcattctctatatcaacggtctgccactggtcgt |
| | | ctttgaatttaaaagtgcggtgcgcgagcaggatgctagtattggcaatgcctggagacaactctgcaaacgctatcgccgggata |
| | | ttccgcaactgtttatctacaacgcgctctgcattattagcgatggagttaataaccggatgggcaacctgtttgcgccctatgaa |
| | | tatttttactcatggcgaaaagtcaccggtaatgaaaaccgtgaacaggatggaattccatcattgcactcaatgattcaggggct |
| | | gtttcatccggtacgtctgctggatgtaattaaaaactttatctgcttcccggataaagccaggcacgaagtaaaaatttgctgcc |
| | | gatatccgcagtactatgccgcccgcaaactctattacagcatcaagcaagcgcgtaaacctttcggtaacggtaaaggcggcact |
| | | tactttggcgcaacgggctgtggcaaaagttacaccatgcaatttttaacgcgtcttttgatgaagagcgtagagtttgccagccc |
| | | gaccattgttttgatcaccgaccgcaccgatctggacgatcagctttctgcgcaaatgtgcaacgccaaaaattacattggtgacg |
| | | acaccatccttcccgttaccagccgtgaagatttgcgtaatcaactggcgggacgcaatagtggcggtgtcttcctgacaacgatc |
| | | cataaattcaccgaagacaccgaactcctttctgaacgcagcaatatcatttgcatctcggacgaagcacatcgcagccaggttaa |
| | | cctcgaccagaaagtcatcatcgataaagaaagcggaaaagtgcgcaaaacttatggctttgcgaaatacctgcacgattcactgc |
| | | caaacgccacctatgttggctttaccggcacaccgattgacgcgacgctcgatgtcttcggtgaggtgatcgacagctacaccatg |
| | | accgaagccgttcaggatgaaatcactgtacgcatcgtgtacgaaggccgtgcggctaaagtgatcctggactccagcaaactgga |
| | | ggaagtcgaaaagtattacgaagagtgcgcaaacgcaggcaccaatgagtggcaaatcgacgaaagcaaaaaagccaccgcaacca |
| | | tgaatgcggttctgggtgatgaagatcgattaaaagccctcgcggaagattttgccaaacattatgaaaaacgcgtagccgaaggt |
| | | tccaccgtaaaaggcaaagccatgtttgtttgtgccagccgtgaaattgcctgggatttctaccgccagcttaaagctattcgccc |
| | | tgcctggtttgaagtgaagcaagcccccgatggcgtcttcctgacagaacaggagcaaaaagagttaccgccttctgaaatggtga |
| | | agatggtcatgacgcgcggtaaagatgacgacgaggcgctttatgatttactgggcacaaaagaatatcgcaaagagctggataag |
| | | cagttcaaaaacgctaaatcgaatttcaaaattgccattgttgttgatatgtggctgaccggttttgatgttcctgaactggatac |
| | | tatctatattgataagcccttacaaaaacataaccttatccagactatttctcgcgttaaccgtaaactggaaggcaaaagcaaag |
| | | ggttagtggtggactacatcggcattaaaagtcagatgaaccaggcactggcaatgtattcccgcattgatgccaccaactttgaa |
| | | gatattcagcaatcggtgactgaagttaaaaaccatctcgatttgttggggcaagtcttttacgactttgacagtcgggattattt |
| | | tagtggtgagccacaagcgcaattatcctgcctcaaccgcgcggcggaattcgttctgcgtacccagaaagttgaacgtcgtttta |
| | | tgggactggttaaacgcatgaaagccgcctacgacgtctgctgcggcagtgaagcactatcacagacagaacgtgatcatattcac |
| | | tattatcttgctgttcgttcaattgttttcaaactgacgaaaggtgacgcaccggatgttacccagatgaatgcacgcgttcgtga |
| | | aatgattgcagaagcgctaaaagctgatggcgtagaagaaatttattttcttggcgataaaaaagcggaatccatcgatatttttg |
| | | acgaagattatctggcgcgaattaacaagatcaaacttccggcaacgaagatccagctattacaaaaattactggaaaaagcgatc |
| | | agcgacttcaggaaagtgaaccagttgcaagggattaacttcacccgccgcttccaggctattatagatcgttataatgagcggcg |
| | | agaagatgatgtactcaacggtgaagaattcgatacattcagtcaggaaatgaccgatattatctatgatattaaaacagaaatgg |
| | | gcacctgggccgatttaggtattgatattgaagaaaaagcgttcttcgacattcttgctcatatgcgcgataaatatcagttcacc |
| | | tatgacgatgaaaaaatgctgtcgctggcaaaagagatgaaaagcgtggttgacaacacatcgaaatatcctgactggagtaaacg |
| | | cgatgatattaaagcgaaactgaaagttgaacttattctgcttctacacaagcataagttcccgccagtagcgaatgatgatgttt |
| | | atatgggggtactggcgcaagcagagaactttaagaaaaatcacatgagttgagtctgtcataatggagtatctcatcagatactc |
| | | cttctttatctattttgtaagagccaaaatagataaattatgttacgcataaccagctcatttaaactatctggtctgtttcctcc |
| | | ggttctacaaaaatagataggggtgcacctacgttaccaatactggcatcatggctacatacggtggtcagtttacgcttactcac |
| | | cattctttacttttttataagcgtcaataggtttgtaagcgactcgtcagaaccgtattgatat (SEQ ID NO: 5) |
| 4 | pLG006 | acctgccttcctttgatacaattcgtaacaggttactatcatcataaaaaagctcaacccgatgaactcgctaaaaatgagacaaa |
| | | tcatttatatctcgaaaaaacttgttacaatcatgagcgctacaccgaacttaaccatataaattatgtgtgttttgtttattttt |
| | | taaacgattacaactatccattatttacacaggtatcaaaatgttagcgcagctttttgagcagttgtttcaatcgatagactcta |
| | | cactgatcaccaatattttcatctgggctgttatattcgtatttttatcagcgtggtggtgtgacaaaaaaaatatacatagtaag |
| | | tttagagaatatgctccaaccttaatgggggcattaggtattctgggtactttcattggtattattattggtttactcaattttaa |
| | | taccgaaagtattgataccagcatccccgtattattaggtggcctaaaaacagcattcattacaagcattgtaggtatgttttttg |
| | | ccattttatttaatggaatggatgctttcttttttgccaataaacgaagtgcgttagctgaaaataaccctgaatctgttacacct |
| | | gaacatatctatcatgaattaaaagagcagaaccagactctgactaaattagtctcgggtattaacggtgatagtgaaggttctct |
| | | tattgctcaaataaaattactacgtactgagattagcgattcctcgcaggcacaattagctaatcacactcatttcagtaataagc |
| | | tttgggaacaacttgaacaatttgcagatctaatggcaaaaggtgctacagaacaaattattgatgctttgcgacaagtcattatt |
| | | gattttaatgaaaatttaactgaacagtttggtgaaaactttaaagctcttgatgcctctgtaaaaaaacttgttgagtggcaggg |
| | | aaattataaaacgcaaattgagcagatgtcagaacaatatcaacaaagtgtcgagtccctggttgaaacaaaaactgcggttgcag |
| | | ggatttgggaagaatgtaaagaaattcctctggctatgtctgaactgcgtgaagtgcttcaggtgaaccaacatcaaatcagcgaa |
| | | ctctcccgccatttagaaacctttgtcgccatccgcgataaagctacaaccgtattacctgaaatacagaacaaaatggctgaagt |
| | | gggtgaactgctgaaatccggagctgcaaatgttagtgcatctcttgagcaaaccagccagcaaatacttcttaatgcagattcaa |
| | | tgcgcgttgccctggatgaaggtaccgaaggattcagacaatcggttacccaaacacaacaagcatttgcctcgatggcgcatgat |
| | | gtcagcaattcctccgaaaccctaaccagcacgttaggtgaaacaattactgaaatgaaacaaagtggtgaagaattcctgaaatc |
| | | actagagtcgcactcgaaagaattgcatagaaatatggaacaaaatacgacgaatgtgattgatatgttcagtaagactggtgaaa |
| | | agattaaccatcaactatccagtaatgccgataatatgtttgattcaatccagacatcatttgataaggctggtgcagggctgact |
| | | tctcaagtcagagaatcaattgaaaaatttgctctatccatcaacgagcagttacatgcttttgagcaagcaactgaacgtgaaat |
| | | gaaccgtgaaatgcaatcattaggtaatgctctgctttcaatcagcaaaggttttgtcggtaactatgaaaaacttattaaagatt |
| | | accaaatagttatggggcagttacaagcattaatttctgctaataaacatcgagggtaatcgatcatggataagattatagggaaa |
| | | caattacctaaaaaagatcaagataatgaacattgggtatccatgtcagacctaatggcagggctgatgatggtttttatgttcat |
| | | atctattgcttatatgcactacgtacgtattgaaaaagaaaaaattaaagaagttgccgtagcctacgagaatgctcagttacaga |
| | | tttataatgctctggatattgagtttgcaaaggatttacaagactgggatgcagagatcgataaacagactctggaggttcgattt |
| | | aaatcaccggatgttttatttggcttaggaagcacagagctaaaaccaaagtttaaactcattcttgacgacttctttcctcgcta |
| | | cctaaaagttctagataattatcaggaacatattactgaagtccgcattgaaggtcacacaagtactgactggacaggaacaacga |
| | | atcctgatattgcttattttaataatatggcactatcgcaaggtcgtacacgtgcagtattacaatacgtttatgacataaaaaat |
| | | atcgcgacacaccaacaatgggttaaaagtaaatttgccgcagtaggttattcatctgcacatcccattcttgataaaaccggcaa |
| | | agaggaccctaatcgctctcgtcgtgtcaccttcaaagttgtaacaaatgccgagttgcagattagaaagattattcaggagtaag |
| | | agatgaaattatctatcgacatttcagaacttattcaattagggaagaaaatgttaccagaaggagtcgatttttttctggatgaa |
| | | tcccctattgactttgatcctatagatattgagttatccacgggtaaagaagttagtatcgaagatcttgaccctggtagcgggct |
| | | tatctcttatcatggccgccaggttcttttatatattcgggaccattcagggcgttatgatgcggctatcgtagatggcgaaaaag |
| | | gaaaacgttttcatattgcctggtgcagaactcttgatgaaatgcgccataaaaatcgatttgaaaggtatcatgcaactaaccgc |
| | | atagatggtttattcgaaattgatgatggttcaggtcggagccaggatgttgatttacgggtatgtatgaattgcctcgaacgact |
| | | taattataaaggaagtattgataaacaacgaaaaagagagatttttaaatcattctcattaaatgagtttttttcagattatagta |
| | | cctgttttcgtcatatgcctaagggtatctatgacaaaacaaatagtgggtatgtcgaaaactggaaggaaatatctaaagaaata |
| | | cgagaaaaggcaaattatgtttgtaatgattgtggcgtgaatttatcaaccgccaaaaacttgtgccatgtccatcataaaaatgg |
| | | catcaaatatgataatcaccatgaaaaccttcttgttctgtgcaaggattgccatcgaaaacagcccctccacgaaggtatattcg |
| | | ttacccaagcagagatggctatcattcaacgtttacgttcccaacaagggttattaaaagcagaatcctggaatgaaatatatgac |
| | | ctgactgatccatcagtgcatggtgatattaatatgatgcaacataaaggctttcaacctcctgttcctgggttagatcttcaaaa |
| | | ctcagaacatgaaattattgcaaccgtagaagctgcatggccaggccttaaaattgcagttaaccttactcccgccgaagtcgaag |
| | | gatggagaatatataccgtgggtgagctggttaaagaaatacaaaccggagcctttacgccagcaaaattgtaaattctaaaactc |
| | | cgtgaaagttaaggctttcacggaagataaataaagtttccctgatttgtgactcaaattacaaaagtagtttatggcataacttg |
| | | tctgatttttatggtgtaacaggtataaaagcatatgctatggttcgcctcatacttaaaacttccctcatatgggtgaaggttaa |
| | | agcttggtagacagaagacagtcacaatgaataaagcaataaattga (SEQ ID NO: 6) |
| 5 | pLG007 | acatcccgtcatcatgccatcacgacgcgctgagacgctgaaaaaataaaatcagcaccaccgtcagcgcgcagtgctttccccgc |
| | | ctcgcccgcccgcttcatgagacggttttaatgcagttgcattatgtcccgctcctcagtgctgcgctccatcctgattacaaaaa |
| | | ccgttatcaaaaacacatgcaaatagacgcagtcaaatgcgctaccgcctctcgcaataccttcaatttcatgataaaaaacatca |
| | | tccctaacaagagcattatcctcatgaaaaaagtatatgaactaaccagtgaagaagcactgtcatattttcttcgccatgactcc |
| | | tacacaacattagaattaccggcttatattaatttcaccacattattaaatgatattaattcatctatccataacaaaaaaattaa |
| | | aattgaaccaaccgccaaggagctgatgggtaaagatatcaattatgaggtgcttgtcagtaaagatggtctatatagctggcgta |
| | | ggataacacttatcaatcccctttattatgtctacttctgtagaaaaatcacagcaccagcaacctgggaaatcataacagaaaaa |
| | | ttcaaatcttttgaatcaaacgacctttttacatgttcaagcatccccgtcagaaaagacaactcgtcaaacattgctgcgtctgt |
| | | aatgaattggtgggaagattttgaacaaaaaagccttgcccttgctcttgaatacgaattcatgttcagcactgacatctcaaact |
| | | tctacccatcaatatatactcatagttttgaatgggtattcatatcaaaagaagaggcaaagaagaaaaaaagcaaaaataaccca |
| | | gggggattaattgacagccacattcaaatgatgatgaacaaccagacaaatggtattccactcggcagcacattgatggatacatt |
| | | tgctgagcttatcttgggtcaaatcgatatagaattaagaaaaaaaactaacgaactcaaaataataaactacaaggtagtacgct |
| | | accgtgatgattaccggatcttctctaatagcaaagatgatttagacataatatcaaaatgtttagtcaatgtattgggcgatttt |
| | | ggtttagatctaaactcaaaaaaaactgaactatatgaagacatcatacttcattcgttgaaacaagctaaaaaagactacatcaa |
| | | agaaaaaagacataagtcactccagaaaatgctctattcaatatatttattttcacttaaacatccaaactcgaaaacaaccgtta |
| | | gatatctaaatgattttcttaggaatttatttaagcgaaagacaattaaagataacggccaacaggttgatgctatgcttggtatt |
| | | atttcaagcatcatggcaaaaaaccctacaacgtacccagtaggaacggcaattttctcaaaactcctcagttttctttatggtga |
| | | tgacacccaaaaaaaattaacaaagctagaacaactccataaaaaactggataaacaacccaatacagaaatgcttgacatatggt |
| | | ttcagcgaactcaagcaaaaataaacctagagtggaataaatcttataagtcagctctatgcgtccgtataaatgatgaactcaca |
| | | aaagagaaaacattttctgtaaataatttatggaatattgactggatccaaggaaaagaaacaagccccaataaagccaaaatatt |
| | | atccttgctaagaaaaacaaaaatcgttgacacagataaatttgataaaatggatgacaatataacacctgaagaagttaatctat |
| | | tctttaaagagcacagcaattaatatcccaaagccatgttagtaacataacatggcttttttaaatcactcattatcagttatcaa |
| | | gaacgaacataacattctattccgaggag (SEQ ID NO: 7) |
| 6 | pLG008 | agttttttaaaggggttattttctaattatagtcccttaatttccattttcgtgtctaattatttgacattagtccatacaatagt |
| | | gactctaagatttaaggataacatcaactttcaacataagcacaataactatttttttattataattgaaaagagaattgaattat |
| | | tacctataaaacttaaaggagtataattatgaaaaaagagtttactgaattatatgattttatatttgatcctatttttcttgtaa |
| | | gatacggctattatgatagatctattaaaaacaaaaaaatgaatactgcaaaagttgaattagacaatgaatatggaaaatcagat |
| | | tctttttattttaaagtatttaatatggaatcctttgcagattatttaaggagtcatgatttaaaaacacattttaacggtaaaaa |
| | | acctctatcaacagacccagtatattttaatattccaaaaaatatagaagctagaagacaatataagatgcccaatttatacagtt |
| | | atatggcattaaattattatatatgtgacaataaaaaagagtttatagaagtatttattgataacaaattttcaacgtcaaaattt |
| | | tttaatcaattgaattttgattatcctaagacacaagaaattacacaaacattattatatggaggaataaagaaattacatttaga |
| | | tttatctaatttttatcatactttatatacacatagtataccatggatgattgatggaaaatctgcatctaaacaaaatagaaaaa |
| | | aagggttttctaatacattagatactttgattacagcttgtcaatacgacgaaacacatggcattccaactggaaatctattgtct |
| | | aggattattaccgaactatatatgtgccattttgataaacaaatggaatataagaagtttgtgtattcaagatatgtagatgattt |
| | | tatatttccgtttacttttgagaatgaaaagcaagaatttttaaatgaatttaatctaatctgtcgagaaaataacttaattatta |
| | | atgataataaaacgaaagttgacaatttcccgtttgttgataaatcgagtaaatcggatattttttctttttttgaaaatattactt |
| | | caactaattccaacgacaagtggattaaagaaataagcaattttatagattattgtgtgaatgaagaacatttagggaataagggag |
| | | ctataaaatgtattttcccagttataacaaatacattgaaacaaaaaaaagtagatactaaaaatatagacaatatcttttcgaaaa |
| | | gaaacatggttaccaattttaatgttttcgaaaaaatattagatttatcattaaaagattcaagattaactaataagtttttgactt |
| | | tctttgaaaatattaatgaatttggattttcaagtttatcagcttcaaatattgtaaaaaaatattttagtaataattcaaagggc |
| | | ttaaaagaaaaaatagaccactatcgtaaaaataattttaatcaagaattatatcaaatattgttgtatatggttgtctttgaaat |
| | | agatgatttattaaatcaagaagaattactaaacttaattgatttaaatattgatgattattctttaattttagggacgattttat |
| | | acctaaagaatagttcatataaattggaaaaattattaaaaaaaatagatcaattatttattaatactcatgccaactacgacgtt |
| | | aaaacttctcgtatggcagaaaaattatggctatttcgttatttcttttattttttaaattgtaagaatatttttagtcaaaaaga |
| | | gataaatagttattgtcaatctcaaaactataattcaggacagaacggatatcaaacagaacttaattggaattatattaaaggtc |
| | | aagggaaggatcttagagcgaataacttttttaatgaattgatagtaaaagaagtttggttaatttcttgtggtgagaacgaagat |
| | | ttcaaatatttaaattgataagtatttgaaatctattattagttcctgaaaaaatagctgtgtcttgtcaatataaatgacaagac |
| | | acagctattttttttaattttgaaatttataatt (SEQ ID NO: 8) |
| 7 | pLG009 | |
| 8 | pLG010 | |
| 9 | pLG011 | gcccatcattgcattaagtgatgggcggagcctttggcctctaatctggaactagctgcgattttcagactcgaatgctaaaaggt |
| | | cgtttcgcacctgaaatcaagctgctagagttctcttacggggttctcccctcgcatacgcgctgtagtaactgcggcgtaagagta |
| | | aatgtctgcacatatcatgcccgccatgatcattcggtaattcctggcgtgactggaagggagaccccgtgccacctatgggccata |
| | | tttttggaccagtgagtttcgtgaagttgccgccggagttgatgagtgaggccagtcttcttgctcatcttggcgttggccgtgccg |
| | | aacttaatgtcattagttggtacgccggtaggatgtaccataaattcgacattaaaaagaagtctggcaaggcgagggtgattaatg |
| | | cgccggatcgtcggctgaagatgttgcagaggaagatcgccgatttgctgacgcctctctatcggaggcgcaaccctgttcacgggt |
| | | tcgtgatcggtcgttctgtgaagaccaatgctcagtcccatctgggcagcaagttcatcgtcaacttggatttgaaggatttcttcc |
| | | cgtccatttcgtacggacgcgtgacgggcgtgctgcgttcgcttggcatgaagcgcgaggtcgcggaagctattgcgacaatttgct |
| | | gcctcaatgggacgttgccccaaggcgctccgagcagtccgatcttgtccaatatggtttgcttccgcttggatcggaggctgcggg |
| | | agttagccaaggacgcccgttgcatttacacccgctatgcggacgacctgagcttttccagctaccagccgctaatgggattgttcg |
| | | aaacgacaccaccggcttcagggcatttctcaccggatctgttgtcggaaaaacttaagcagattttcagcggtaacgggtttgtgc |
| | | tgaacccggacaaggctcactatgctgacaagcattcgcgccgcaccgtgacaggcatccggattaacgaggctctcaatgtcgacc |
| | | ggcggtttgtgaggaatttgcgggcagccctttactctgttgaaactttgggactggccgccgcccaggcaaaattcaaatccttgc |
| | | atggtggtaaagctgacgtcggccagcacctgcaaggcaaggtatcgtggttggggtacatcaaaggcgcatctgacccagtctttc |
| | | ggagtgtcgcatcccgtttcaacgctgcattcccgccgctcgcgctcgatattttgcccagtccccaagaaatacgagaacgatcag |
| | | tgtggctgattgagcactgggaaacagggggtgaccaaggcacggcgtttttcatgaagggtgtcggtctggtaacggcagagcatt |
| | | gcatatcgccgtccggtatagttgagttgtatcacccgacgaagccgtcgaataaattcgcggcgtccgtgaagcatcgatgcccag |
| | | atcgcgatctggccgttctcgaccatgcaatccccaacaacgaattctatgagctcgaaaccgccggcaaggcagccgcgacaggcg |
| | | atgccacgaccgcgatcgggtatcccggttatggacccggcgacagactgaacatccgacctggcgcagttacgtccctgccaacta |
| | | agagtgcggtgaagatggtcgaggtccagcagatgctgacgccgggcatgtcaggagggccattgctggatgtggatgaccgcgtcg |
| | | ttggcgtcgttcacaagggcggccatgatcatggtcggcaactcgctattgccatatctgaactgcatgcttggctgccctgacctg |
| | | attagccgaaccggctaatcgcgcaggcgccgaaccagccgtttccagcttgcttcactgttcatccagtcaggccggtccggttgt |
| | | cgaggcgttggagcaaatcgttcaggatgtccccgacagcgcgtgcagcgcaggtgcgatccgacggtttccatagcggtgttccag |
| | | caatgcgcgaggaaccagcggttgagttt (SEQ ID NO: 9) |
| 10 | pLG012 | tctatctaaaagtatacatatagtatttcaatgaaggttatattatattttgtggctgttttctaattttatcaataagattattg |
| | | caaaaggctgataaatataatagctttattatatcggaggagttgatttaactttcctatactatctgtataggctaataccaatg |
| | | gcaattttgccctcaaattggtctccttaatgtttatcaacgtgttatacggtagtgataaaacctcctccgatatttttctcatg |
| | | aattgggatattttaaatatgttttgctcagtaaccaagttgcatgaatgtaaaaatgttgaacaattatactattttttaggatg |
| | | tgaagaggctgaaattagtaggtttttatatagtggagtaattaaataccgctctttttccatacttaaaaaaaatggtaatttta |
| | | gaaatataagagcacctgtaaagtatttaaaagaaattcagtataagataaaggatgagctcgaaaaatattataccccgaaatca |
| | | tgtactcatggttttatagctggaaggaatataatcacaaatgcgaaacctcatataagaaaagaatttattttaaatatagattt |
| | | aaaggatttttttgattcaattaattttggacgagttagtcgtttatttcaaagccaacctctaaacttgccagagaatgttgccc |
| | | atgttttggcacatatttgttgctataatagagccttacctcaaggtgctcccacatccccaattatatctaatatgatatcttat |
| | | cgtttagacagacaattgaaggagttggcaagaaataatgcgtgtacttataccagatatgcagatgatataactttttcttttac |
| | | taaaactaaaaagtatcttccaaaatcaattgtttctttaagtaaagataataacattatactaggccatgaattaaaaaaggtaa |
| | | ttgaagataattggtttgaaataaatgaaggaaaagtaaggttacaacataaaacacaaagacaatcagtaacaaatattacggtt |
| | | aacactaaaattaatataagtagaaaatttaaaaaacaaacttcagctatggttaatgcattatttaaatatggagcatctaaagc |
| | | tgaaagagaatattttagtaagtatcacaagggttatatagcagaaaggcaatataataagattaaagaaaaaccaggtttattat |
| | | ttacacaaaaagtaagaggaaggttgaattatatccgattagtttgtggtaagaataatgaaagctggagaaagctcatgtataaa |
| | | tatactgtggcaataggacaacctaatgaggagtacaatagaacattgtgggatattgctggtgattcaacgttcattctttggtc |
| | | gaattcctcacaaggaagtggtttttttcttgaaaatattggtttagttacaaatgagcatgtaatcgaaggaatagaaaacagca |
| | | atattaataatgatctaataatactttggttaccaaatgaaagaaaagaatatattgagttacacttagcttggaaagatgataat |
| | | actgatttagctgtaattacttctaatatatcttttcttgacataaagcctttacaagtagagccagttcctatttatgatatagg |
| | | aacagaagtatatgcagttgggtatcctaattatgacgccagaggctcaattggaaaacctactattattacagcaaaaataacga |
| | | gtataattactcgagaaaggcaagaaagaatcgttatagaccaaccaatagtacatgggcatagtggtggggtcgttttaaatgct |
| | | gatggacgtgtaataggcattgttgcaaatggaaatgccgagggggaattaagagtagttcctaatgcttttattcctattgaaat |
| | | attattaaatgagcacaagttacgaactaaatcataaaattattattcttaaaataattaaatattttttaaaaccactagtttga |
| | | taactagcggttttttatttttggagtacat (SEQ ID NO: 10) |
| 11 | pLG013 | |
| 12 | pLG014 | ttataacaagcatttatagtttaaagatactttttctaatcaagtagaacctttgggtggcatcggcctatctcgcttttgtccaa |
| | | atgtgggctgatggggcatgaaaaatggaaatgccccattcctacttagtgctattactcattcatacctcgttaacgtgattttg |
| | | gattagttttattcactgtatatatcaacagttataatgaagcgcggtgattttatcgctttagttctgtttttaataagaaatat |
| | | ttcttgttaaaaacagaagtgaaatcataactaattgaaaattatatcgtttaacatttcagtttgtatttaataagactgattaa |
| | | atacatttcttacttttcacaccctctttcaaatcggtgagtataagaaagtgccagtaagctcataatatttaacgattatatcg |
| | | agtataatatctatcttttataagtatatttttgcgtaaaagtaagaatgcttattaatatactgttagttgcatcaagtgatgca |
| | | ttgcattctgtttagtattgttatagattctgccgcaagaggcgagagtttaactttctgctgttaatctgcggcggtcataagca |
| | | tgtttctttttaccggttttcagctagtctgatgatgccgttacgctgtacaagagaaaacaaaatcgcctcgttctttaagggtt |
| | | tgttactttggtagacatttcattaatttcccaaattgcagctaaagctgcattctcatccaatattcaagtacctctacctaata |
| | | aattgaaagattgctcatgcgttgaagggctgactcaatatctgggttttacgaattatgatgagctgaaaaaactgatatacccc |
| | | tcagttgaccacctatataaaggctttagcattcctaaaaaaaatggcgagtttcgaacgattgatgcgccaaaaaaggagctaaa |
| | | aacaatacaaagtttcctttcgaaggaattggttcaagtttactctcctcgtaatgctactcatggttttgtaaaagatcgaagta |
| | | tagttacaaatgcgtcgaagcatgtagacaaaaaatacgtactcaatttagatcttgaggacttcttcggctcaattcattttggt |
| | | cgcgttcgaaacctgtttcaatcgcatcctttgaacttacaccattcggtggcgacggttttatctcacctatgctgccacaatgg |
| | | caagttacctcaaggcgctccaacatccccgatcatctcaaatatgatcgcttatcgtttagacaagcaactgcagacattggctt |
| | | ctaaaaatagatgcacatatacacgctatgctgacgatataacattctctttcacacaaactcgtgggcgcttgcccaaatctatt |
| | | gttacgttaactcgcgatctacaactctctttgggtaatgagctaaaggagcttattactgagaatggttttgttatcaattctga |
| | | taaaactagaatagctgcgcgaagtaataggcaagaggtcactggtgtgatcgtcaatgagcgtatgaatgtgtctcgaaagtaca |
| | | ttaaacaaacacgttccatgctatatgcatggaaaaagtttggtctcgaagatgctgaagaaacctacttgagaaagtttcatgga |
| | | aaaacagtgtttgagaagcaccagcggcgaattgacgaaaagaaagggcagttttttaagaaagttgtaaaaggcagaattaactt |
| | | tattaaaatggttcgtggtgctgaagatttaatatacagaaaaatagcttacgaattctctgtattaattagcaagcctaaaccag |
| | | agcttgtgcaaaccccattggataaagcgtgtgattcaatatttatcgttgaaaatatggtggagaagagccaagggacagcgttt |
| | | ttgctgaagggaattggtatcgttacaaatgaacatgttgtgcgtggaatcgatgaggaactgtcagatcttttggagctatttag |
| | | gtatcatgagcaggaaactaagcgtccagttaaatttcaaaagtcatgcagatctagggatttggctattctaaaaccaactacaa |
| | | gctacaacggtattaagcgcttggatgttggtgatgatagtcagatcggtattggttcggttgtaaccgtcttaggttttccccag |
| | | tattcgcctggtgaaacgccttatatcaatacaggcaaaattatccaatctaaagtattgtttggtgaacgcgtctggttgctaga |
| | | tatacctgtaatccatggaaatagtggtggccctgttcttaatgaccgtcaagaagttatcggcgtagctgcaataggttcgccaa |
| | | cacatgaccactcaacgaaactccatggcttcataccaatttccacgttattagcgtatgtggaagaatgcaactaacaaataagg |
| | | atatgtgtcgcgaagccgacacctatccgaagtgttggacaagcccaagccaccttatataagtaaataccatcaagagtaatgtc |
| | | aaatccttacttttcctaatctctaaaagcctaaatagaacgaacggtctaagaagcttttgtccaacaacgagctagcttatgtg |
| | | atagctagtttgtgatcaaactttagatttttacactctacaaatagcttgaaaagtcacatttccgatcagactta |
| | | (SEQ ID NO: 11) |
| 13 | pLG015 | |
| 14 | pLG016 | cgttaataattatgttgttagcttaccacatttcattatcataaatacttacagtaggtaagataatgtaaaacatcgcgattaaa |
| | | tataaacttttcaaaaatgctgttaatattgatgaatatatatagtataatttacactgacagcaagggtaagaaaaaattgactt |
| | | tatggcggtgaaatcgccgtctgttatttaaagggtatacttaatttacacgcttattttatcttcgaagttttattcgatttgtc |
| | | taatcgctattaggagaagggtagaattttaacccttgctgttgtaaataggaggggattgctatggtttataagttaaattttga |
| | | attacagagcaatctagaggatattaaacaaaatttcaagaatttatcttgttttgaagatgtagctctccttttagaggtaccaa |
| | | aagaattattgtggaaagtacttataaaaaataaaggagctaattataaggcgtttaaattaaaaaagaaaaatggttcagaacgt |
| | | gttattttttcgcctactttaagtttatctattctgcaaaaaaagctagcttatattttggagtctaactataaaaaccataggca |
| | | atcatatggttttgtaaaaggaagaggaatagttgataatgctcaaaagcatttaaataaaaaatatgtactaaattttgatatag |
| | | agaattttttcgaaagtataacctttagaagagttagatcaatgtttatgacatattataaatttaatgaaaaagttgctacaacc |
| | | ttagcaaatatatgttgtcatccgaatggttttctgccacagggagcagcaacatcccctatcatatcaaatattatatgtaatag |
| | | aatagataaagagttttctaaattggccaaaaacaacagatgtcaatatactaggtatgctgacgatataacgttttctacaagca |
| | | ggagggttttccctcatgatattgcatatataaaagaggggtctatttttctgaatagtaatgtaattagtattgtggaatatcag |
| | | gggtttaagattaataaagaaaagacaagacttcagaattatagacaaaatcaaactgtaacgggaattacggtaaatgaaaaatta |
| | | aatgttaaaagaagctatgtaagaagaataaggtcaattcttcactgtattgaaaaaaacgttgaagatttacagaaagcagaacaa |
| | | attttcgaagaaaaatacccatttcgtcaaaagaaatatcttgataatattaatatgtttgctattttaaaaggtatgatttcaca |
| | | tgttgggcatgtaaaaggaaaagatgaccctttatatttgaaattagcaaagagatttaataaaatatcttatcttagtgaaacta |
| | | tatctccttttaaattagaatctttaaagaaatttcatgaaacttatacatatataattgattatgatgataaagttcctttagtt |
| | | tgttttgaaaacgataaaatggaggaaatattatacggtcaaggaacgggctttttattaaagggagttggcttaatcactaatgc |
| | | tcacgttatagaagatgcaatagaagctattaaggacaataaaaaatttaacaatgagtatggtatctcattttttagaggtaatt |
| | | atcctgatttaaaatataaagcgaaagtatccaaatatgacctagataaagatattgcaattttagatataaaaggttttaatata |
| | | gacaatcaaggatatgaatataacattgacatgaaagatgggcagaaaattgaattaatagggtatccagactacaaaatagggca |
| | | agaaataaaaatcgaaactggccacctaaaaggtattagaaaacatagagattcaaccggaacgttccattcacgacgggaaatat |
| | | cggcaatcatatacggaggaaacagtggcggacctataataaatgaaagtaatgaagtcataggagttgcagttaaaggtgctacc |
| | | cttcatggtgtttccccaagcgagattattccaattgaagatgtaattaatttaaactccagtaactcagaggtcagctccaagat |
| | | tgcaactaagcctcattaaaagatttaatattttaatgcgaaaagtcgatttttaatcaatctacttttttatttttcattttaag |
| | | ttgtaaatatctcttacaatttattttatttcaacgacatatttgggtatc (SEQ ID NO: 12) |
| 15 | pLG017 | gtggcaagattataccccatcaggcataagatgctttgacttataacgcatcagtttgaaacacaatggtgatgggggtcacaggg |
| | | gctgacatgtacttttaagattaaaaagcattaacatctacttttgaagaaaacagaaaaaaacaatcacaaacctttaaaaacaa |
| | | aaactatgccaattattaataaaaagtatcaagagcttcagttaacagatgagtacattaccgatccactgctcatggccctagcc |
| | | tggaagaaaagccatcactacatacgtaccacaaattggtatgctgacaactttgaactagacctgtcggctttggacctaatgca |
| | | gcactgtaaagattgggtcaagagaatgcaggacaaaaaagaatttaaattttcagagctacaacttgttcctgtaccaaaagcct |
| | | gtaaatgggagtttaagactgtcgaaaataaggttctatggcaaccttgtgatgaaaaagaacttaccctacgcccccttgcccat |
| | | atacccatagctgaacaaaccatcatgacattagtcatgatgtgcctagccaatacaatagaaaccaagcaaggaaacccagacac |
| | | cagctatgacatcgtccaccagaaaggtatcgtcaattacggaaatagactttattgtcagtatattgacgataaagcagagcaca |
| | | gcttcggtgcaacagtgacatatagtaaatacttcactgattatcggaaatttttaaataggccttatcattttgcgtcaaaagcg |
| | | caaggtgaaatttcgccggacgaagccgtttacatcatagaactagatcttgcgaagtttttcgatttagtaaacaggaagactct |
| | | aattcaaaagataaaaaaccatatcagtgagtcaataaacaataaagaaaacccactcgccaatcatttatttaaatgttttgcaa |
| | | actgggactggactgcatctagcataaaaaattatgacatatgcaagtcagacgaagtaacagaaataccaaaaggcatccctcaa |
| | | ggattggttgcagcagggtttctatcaaatatttacttacttgaattagatcaattcttgcataataaaattaacacagacataac |
| | | tgatgacattaaatttgttgattactgtcgatatgtcgatgacatgcgatttgtggttaaggttaaaaaatcaaaaaataataata |
| | | ccgcattcataaatgatgtaataaccaatcttcttaaaaatgagatagataatcttggactgataattaatcctaaaaaaacaaaa |
| | | gtagaaatttttagaggcaaatccgcaggcatctcgcgtagcttggaaaacatccagaccagattaagcggcccaatatcaatgga |
| | | tagcgccaacgaacaacttgggcatcttgagtcattattaagtctgacaaaaaccgattttgaaccaccgaaaaatggtaaatcaa |
| | | atagattagctgagattgaaaaagaccgtttcgatgtcagggaggacactcttaagcgcttttctgccaataaaatcagtaagata |
| | | ctaaaagagttaagacatttcatctcgcaggatatagatactgatggggaggttattgccggggaatgggattatctgcaagaacg |
| | | tttggcacggcgttttattgtctgttggagccatgacccgtcactggcactgctactcaagaaagggctggaacttttccctgatc |
| | | ctaagctattagaccctatacttgaacagctttgctcactcattgaaagcgataatgaaaaacaaagtgcagtagctacttattgc |
| | | cttgctgaaatatttcgacattcagcaatgactattcataaaaaagacacctatgcattccctgcacaagccaatgtggatgggta |
| | | ctttgaaaaaatacaacattgcgccgcgacattcattaataagcgcagcgcctctgacaacgaaacttggaacctgttaattaatc |
| | | aggctagttttctgttgcttgtgcgtttagataatacattagaaaaaaatggcactgatgccaggcatgatcttatcttaaaactg |
| | | gcatcaggctttagaacaattacacttcccactaaaatggatagcaagactatagcctcatgtattttgttggctagtcaattagt |
| | | taaagataacaaaccatttattcgctcctgcgcttctttgtgcgaaagaatttatgacaaagaacacgtcataaaattgaagaaaa |
| | | tagttagcataatatcacatcaaaacttatcattgtttaaatccttagtttatcattcacgacctttacaacagaagtggctaaac |
| | | tcagactccgtgaaaataataattaatgaatgccatatagatatacaacctttggcgacttctttaggcatgataaaaagtagtca |
| | | ctcattacttagaatcatatcaagacctgataacccatttgccaatgagataatggcattaaaactgatgcaagcccttttattgg |
| | | acaggattgtttgcctggataataaaaaagattatcaaataagtgtagcaaacaccaaagtgacgtttcataactactccaaccct |
| | | ccaacatcgaatgtcttcgatgcaggaatggatatggatgcaaaattattcaaatcatcgggatgggtcgattctattttcacgga |
| | | tgatgcagacactcaaatattgtatagagttgccatgtgcatccgttcagtactactcggcaaacaagactggacagattttggtc |
| | | aagcaatitcccccaaacagggttatcggggiattaaaactagtagagacaaacgtcaattggggatgatgacaacacctgagtcc |
| | | attgccggtgagaactctcaggtttctggttggcttaccacactcttatccaagttgcttgcctggccgggaatttcagtgggtga |
| | | taatggatatcaatggccagcaatttttacagtagatgctgtcagaaaactagttgatgctcggctgagtaaacttaagcaggatt |
| | | actgcaaactatcaggaactccgggacttacagaaaaaatacagttcaactggtctgactcgaaaaaagccctaacagttgctatg |
| | | gtccagtcaaaactgcctgcaacgaaagattttgtcagccatggacttcttttaaactccgcaaagtatagagtgattcatcgcag |
| | | acatgttgctgaagtggctgatttagttgtaaaacacacgcttgcacaaaaaacaactcaacgaactcatggtgaaaaaatagaga |
| | | acattgatttaatagtatggcctgagctcgctgtacatagtgacgatttggatgtactcatcgccttatctagaaaaacgaatgca |
| | | atcatatactcgggcctgacatttattgagcaacctggaatcaaaggaccaaataattgtgccgtttggattgtcccacctaaaag |
| | | caatagcagccagaaagaaatgataagacttcaaggcaagcataatatgatggaagatgagaaaggccgggttgaaccctggagac |
| | | cataccaattgatgcttgaacttgttcacccccaatttactgataaaaaaggatttgttctcacaggctccatttgttatgacgca |
| | | accgacatcgcgctaagtgcagatctcagggataaatcaaatgcttatcttgtagcagcattaaacagggatgttaatacattcga |
| | | ttccatggttgaagcactgcattatcatatgtaccagcatgttgtgctcgttaactcaggggaattcggaggatcttacgctaaag |
| | | caccttacaaggagccgtttaatcgtttgattgctcatgttcatggcaatgatcaggtagctataagtacgtttgaaatgaacatg |
| | | tttgatttccgtcgtgataatataggaaaaagtatgcaatccgggttagataaaaaaactgctcctgcaggaatcataatgtaata |
| | | aatattagatatttttatattagaggtgaggagatggcgtcacctctaatattttcgctgattgtatttagcatcaaataataaag |
| | | gtacaattaatttaagtgactatcatgaaaaaattagttccgccatatcaagtaaccccggcacaaatctatcgttccgttgccag |
| | | ttctacagccattgaaaccggaaaac (SEQ ID NO: 13) |
| 16 | pLG018 | gcttatcccctccctactggtaacagcgttatcgaacttggaataccatcatttatacctatatctgttggtagatgtgcattgaa |
| | | gtgggttgaccttgagagagccagtatcgcgggcgcaggaatgacaggtaagcactaaatttcaggcacaaaaaaagctgccctta |
| | | agcgacttgattgtatcttttggtgcgaaggccggactcgcacataaaacttaacctcatgatttaaaaaagataacaaaaaacag |
| | | tttaattttataccaacacagataccaacacgaaaattcattgttcttgggtatcgaacccggacaaacatgactgagttgtatta |
| | | gctcagatttgacctgacacagttatggcacagatctcaacctaatctgacaggcagctccgtatcagaagcggaagtgatgacca |
| | | agtttaagcatcattcttggcttgtatgagaatggcactgatctagcgatcagtaaaacttcatcgcttcatcgaaatgccctaaa |
| | | actttagattaggagaaagttctatttatgccagctacaatttttcgggggagttaccttaccgctaaataaaccgaaaatcgatg |
| | | ctggacaatctctaactcggtggtcaattttcgttgaccactacataatggtcctcctgatgcatctgatgtatcaggaggaccgt |
| | | ccttaaacacgacaaaacctgtgatacttaccatggattcctctatgaaggaaaggtagtatagccattttgggtgatacatacag |
| | | tgaatgtcattgctgtagttgaagtgagtaagagcgcttaagattaagttgagagaaaatgaaactacttgataaaaagtattaca |
| | | acctcgagcccaaatatgagtaccttaaggactcatttattttaggactggcatggaaaaaaacagatagttttgtaagaactcac |
| | | aattggtatgcagatattttagagctggacaagtgtgcgtttgatattagtgatgaagtcactaattggtcaaacgagatctcaaa |
| | | gaacgctctttccaaaagtgatattgaattgataccggctccaaaaggagcaagctggttcattaatcaaggtaaatggactacca |
| | | ataaagataatagaaagataaggcctttggctaacatatctattagggatcagtcttttgctacagcagtaacaatgtgccttgct |
| | | gatgctatagaaacaagacagaaagactgttcgttgagcaatcttggctatgctgagcatgtaaagaacaaggttgttagttacgg |
| | | aaataggcttgtctgcgattgggacaatgaaagggcaagatttcgttggggaggaagtgaatattataggaagttctcttccgatt |
| | | atcgaagctttctacaaagacctatctatataggcagggaaacagtaaataaagttagcggaattgatgatgtatatatcatcagt |
| | | ttagatctgaaaaattttttcggttctataaaaataaaccttctgttagaaaaaatcaaaaaaatatccgctgatcattatgcagc |
| | | taaattcataaatgataatgaattttggactttggcgaatcggattttaagttgggattggcctgaagaatctttatctttacttg |
| | | agagtttggatataaaagaaaaaaatgttggtcttccccagggattagcttctgctggtgctctggcgaatgcatatctcattgag |
| | | tttgatgaatctttaatttctaagcttcgtactaagatagaagacagccaaataatactgcatgattattgtcgatatgtcgatga |
| | | tattagattagtgatttcaggagaagcactagaaagtaataagattaaggaatctattcatgcattagttcagggcattcttgatg |
| | | agacattggctcaaaatccgtcagataatgaaccatatttaaaaattaacgatagcaagacttatattcttgagctttcagacatt |
| | | gacaacggaagtgggcttacaaatcgaatcaatgaaattcagcatgaagtaggagcttcgagtatcccagagcgtaacggactcga |
| | | taataatatcccggcacttcaacaattattactgaccgaacaggataatttttccgaggatgttgatagtttatttcccgggttta |
| | | aaaatgataagtcgataaaggtagaatctgtacgtagattttctgcccataggctggaaaaaagtttggctaaaaaaagcaagcta |
| | | atttcacctgaggagaggaaacaatttgataatgaaacctcactgattgcaaaaaaattattaaaagcttggctaaaagatccatc |
| | | aattatggttatcttccgcaaagcgatagctatcaatcctaatctagatgcttatagcaccattcttgaaattattttttcaagaa |
| | | tacaacgcaatcgtgataaacgagataaatatataatgctgtatcttctttctgatatatttcgtagcgtcattgatgtctatcga |
| | | aacctagaatcagaatacgtcgacgattatcaaaaattgatgggtgaagttacattgtttgcccaaaaaatactttcctgcaaatc |
| | | ttttattccaaattacgcatatcagcaagcattattttatctcgcagtgatcaataaaccatttatagctagtaataaagcttctt |
| | | ttgatcttgcaaggcttcaatgcgtcttaattaaacagcatttagaaccgttgaatagtagtgatggatacctatttgaggtatct |
| | | gctcaaatcagtaaagactaccgagcaaatgccgcttttctactttctcatacaaatagtaacaaagtagtagacttaattatcga |
| | | aaaatttgctttccgaggaggtgaattctggaatgcaatttggaaagaaattgttaggatgcaagataaagataggattaacgaat |
| | | ttagatgggccatatcaaaatatgagtcaaagccaaatagttcggagcactatctttcatcagtgatcagtttcaaggaaaaccca |
| | | tttagatatgaacatgcgcttctcaagctaggtgtagcattagttgaactctttgatgatacagagaaaaacgtatggcaacctga |
| | | tggtaagcagtattctccacatgaaataaaagtaaaattagaaggtaactcaacctcatggggtgaattatggcgtccaaatttta |
| | | gtatttcatgctcgatagataagaaaggtgaacctggtaaagacccacgctatataagccctgagtggttggcaaattatccacag |
| | | actcaaaatgatgaacaaaaaatctattgggtttgcagtgtgctaagaagtgctgctttaggcaatgtagattatactcaaagaaat |
| | | gatttaaaacttgataaagctaagtatgatggtatccattctcagttttacaagcgacgtatgggaatgttacatacaccagagtca |
| | | attgttggttcatatggaactataacagattggtttgcaagttttcttcagcatggattgcaatggccaggtttttcttcttcgta |
| | | tataagccaagaagatatattgtcaattactaatattattgagtttaaaaactgtttattggaacggctaggctacttaaataagc |
| | | agatatgtatttcatcgaatgttccaaccttaccgactgttgtcaacaggcctgaattagcatctaaccattttagaattgttacg |
| | | gttcagcagttatttcctaaggatactaatttccatccttctgacgtgactttggctaatcccgatgtgcgctggaagcacagaga |
| | | gcaccttgcggaaatctgtaagctaacggagcaaactttaaatgcaaaacttaaaactgagtctagggaacatacaagcacagctg |
| | | atctaatcgttttttctgagttagcagttcacccagaagatgaagatatagttagagcactggcatttagaaccaaagccatcatt |
| | | ttttccggctttgtcttctgtgaacaagatggccgaatagttaacaaagctcgttggattattccagactcttcagagtctgggac |
| | | ccaatggcgtgtccgtgatcaggggaaacatcatatgaccagtgatgaagtggctcttggcattcaaggatatagaccatcccaac |
| | | atattatttcaattgagggtcaccctgagggaccatttaaattaactggtgcgatttgctacgatgcaacagatataaagcttgcg |
| | | gcagatctgagagatttgactgacatgtttgtcattgcagcatacaataaagatgtagacacatttgataatatggcttcagcact |
| | | acaatggcatatgtatcagcatattgttattacgaatacgggagaatatggaggctcaactatgcaagccccgtacaaagagaaat |
| | | atcataaattgatttctcatgctcatgggactggtcaaatagcaattagtactgctgatatagatttagcagcattcaggcggaag |
| | | ctacaaatatataaaaagaccaaaacccagcctgctggatacaatagaaaacattaaggatttttatggatactttagttaagtta |
| | | gctacaattatttctccattaattagtgctggagtagctatttgggcaattttggttgctaaaaaaaccatcagtgaaagcaaaga |
| | | aattgccaagaaaaccatcgctgatacggcctaccaagcatatttgcaattagccatggagaacccacaattttcgaaaggctaca |
| | | gcgcagattgtagacaggagcgagaccctatgtatgatcaatatgtttggtacgtggctaggatgatattctgctttgagaaaatc |
| | | atcgaggttgaagtaaacttaaaagatagttcttgggcaaatacgttggaaaaacatttgaagtttcattctgaacattttaagaa |
| | | aacgaatgttgtcgaagaggctctctatattccccctattttggatctcataagatgtgcagctaactaataacttatcccaatag |
| | | gattatattccacacgataagcccactggaaaatgtaacatcccaagatagtttttgggattgtttcccagtgggcggaaagtatc |
| | | atgatagttgtcacccccggtggagctgcaaagatttttatggggtgggtgttacattgcgcgataaatttgaaatcgtggcttta |
| | | atttctgcttcttgctcaaaagcagactgtcagatttgattgtgtgctgccagtgagaagcgtcagatcaagtctgagctaataca |
| | | actgagttaagatgccgaaatctg (SEQ ID NO: 14) |
| 17 | pLG019 | agggatacgccacagcaagaaatagtttacttattcctcattttgtcgactaaaaatcgacattaaacaaaaaattcaaacttaat |
| | | cactttcgggaaaaatgtgacaaatatatgctcggactggttgcggggagcgtgtaacatggatacaaatcaaaattattgccagc |
| | | ctcactgatggattactggtgtcaagagccccccttcgggcatgaaacggctggctaattctgtacagactgtaatctaaggacga |
| | | taacgcatgacatatcaggcaattttcactggctgggatgatctgacgattgaagaccttctggtcgcttaccggaaagcaaaagc |
| | | cgatagcttctttgagaatacatttcctgttgctatcaaatttgccgagtatgagcaggaattacttgaaaacctgcaaaaactct |
| | | tagatcttttgcagagcgaagatggattcagtagcaataagaagttgattggcaaatttcgtttgttaccgaaaaaattaaccaca |
| | | aagaaaaaacatgaatcccaaaatggacacgtccacttttctaatcctaaacgagcagccgaccatttatttaataattttgatct |
| | | gataccagagtttcgtattattggtgacttcccggttgatagtcacattatctctgcactatggattaacatggtcgggcataaat |
| | | ttgatgccagcttagataactgttgctatggcgcgcggctaaagcgtattcgtaatgatgaattatttagcaatgagcaggataat |
| | | ccattccatatcagtgccgtgggttcttttagcccctacttccagccctaccaaaaatggcgtggtgatggcttaaaagctatacg |
| | | tgacgagttggaaaaagatcgtgacattatcgccgcctcactggatttaaaaagttactatcattttattgatccactggctataa |
| | | cctctgatgatctctataacacactaaacataaaactgactgaggatgaaaaagcgtttactgcacagttagcagtattcttaaag |
| | | cactggtctgacggcgcagcggcatttggaaagaaaatagcgtacaaaacacctgttattaatggtggtctggtcattggattaac |
| | | agccagtcggatcatttcaaatatattgctacaccattgggataaattagtcattgaaaaactatcaccaattcactacggtcgtt |
| | | atgtcgatgatatgttccHgtaatacgcgatacagggacaattactaataatcacgaatttatgttattgctgcaagataggcttg |
| | | gcaatgattgcgtttatttgaaaaacgagcaaaaacaaatatggcaaatacagcagggcgagcatttccagggtaagaccaccatc |
| | | cagttacaatccgataagcaaaaacttttcgtgcttcaagggagggctggaatagacctgctcgacagtatcgaaaaggagatcta |
| | | cgagctttctagtgaacaccgcttgatgccttcaccggatcaactggaacactccaccgcagctaaagtcctttccgctgccggta |
| | | gtgtaggtgaaaatgccgatactctgcgccgtgcggatggattaaccattcgtcgtttgggctggtcactgcaattacgctacgtt |
| | | gaaacactggcacgagatctgcctccaagtgaatggaaagaacagcgggaagagttttatcagtttgcctacaaccatattcttag |
| | | ggctgataatctatttgcacattttagttatctgccaaggctgcttggctttgctatcagtatgaatgaatggcagcacgcggaaa |
| | | aaattgtacttaaagcttacgaatccatcaacctgttggcatcggtgattacttcaggtaaggaagtgaatataaatggttgcaaa |
| | | actcgagcagtaaatgatctttggcgctgtataaaaggcacattaagctggctatttgttgatgcagcgacacgatattacagtcc |
| | | tgacagattatttcttgataaacgttcaaagaaagaagagtgccttgcggatacattttttaatcatatttcacaaagtctgacga |
| | | atctaaaggatttactggatcttcgctttgattcagcagatttttatttaaaagcgccattggtagctcgagctgatttagcaaag |
| | | gaaccttataaacagatcgtaaagagtcagtcggcagaaaaacttgttaatcagcgtgatagtaaaaaagaagttaaaatactgaa |
| | | attaatgagcgactcatcgcttattgatattgacgttattaagctatttttgaaatcaaccaagaatacccgactggaaaaagtgg |
| | | ctaaaggaaatcgtaagaacgaaagttacctaccttacattttccctacacgtcctttaacacccgctgaaatatcagaactggcc |
| | | cccgaatgtgttggattaccctccacatccgacaaaaaaccagatgagagaccgtccaccatttgggcaaaatatactcaagcatt |
| | | acgcggagtatggatcaaaccgacgttgctagcatcggagcaggactcagatgaagcgacaaaaaaagctcggcctaagaaattca |
| | | ttcatattggcacagacaggaaacataaagttgtcgttgcgctaaccagcattaaaacagaggaggacgactgggctaaaatggcc |
| | | tgcaataaatctaacttgtcccgttcaaggtaccagcggatttctgaactggttaatgcaacattgaaactatctcctaaacctga |
| | | ttatgttttattccctgagctttcaatcccgttacgctgggttaacagtattgctgatcgtttgagttcggcgggtatcagtctaa |
| | | ttgcgggaacagaataccgccacttagacgataatcaactgaagagtgaggccgtacttgtcctttcagataacagactcggctat |
| | | ccagcgagtgtcaaaatatggcaacccaagctggaacccgccgtaggtgaagatgaggcattattttcaatttatggtaagtcttg |
| | | ggattcgacacttaatgttaaacaacgtaagccggtatatattcatcacggcgtcaattttggcgttatgatttgctctgaactcc |
| | | agaatagtaaagcgaggatccgttttcagggcgcactcgatgcattaatggtattgagctggaataaagatctagatacgtttgca |
| | | tcgttgattgaatcagcagcgctggatattcatgcctatactattttagtgaataaccgaaaatacggcgatagtcgcgtacgttc |
| | | cccggcaaaagaaccctttatgcgtgatattgctcgtgtgaagggcggtgataatgactttgtggtcgctgcaacgctggatatcg |
| | | actcgttaagggcatttcagagcagggcaaaacgctggcctaaaggcggcgataaattcaaaccgttacctgaaggattccagttg |
| | | gcaaagaaccgcaaaaagctaccgccaaaataagaaactgattttcgctattaataatcagggtatttttgcgtgagatgttggta |
| | | aacatgatgtagcccttgccactcatgaccaatcgcagtatctttctcccgcgcctgcaaaatcaggcgtcgggattagcctcctg |
| | | aagaaatcttatcggcgacacatgacgcgccagcgtctttttttgtgttgttcgcacggttacatc (SEQ ID NO: 15) |
| 18 | pLG020 | ttttcaaaggagtttcgctttccaaatatacaagaaatcattatttctaaaggtatctataagtggatgattcgttttattggaac |
| | | agttgcattctcgttaattaaagcggctgcttccgaccggcgaatggtcattcagaagctgagaatgtggttattttttaaagagg |
| | | aattggcatgattattagccttgaagagcttggccttgcctaccgaaaagcaaaagtcgatctgtactattcatcccatgtttcgc |
| | | tggaagcaattgcgtcttacgaagagtccctacatacgaatctgacggttctgcaggaaaaaatacaaggtgacgacgaatcatgg |
| | | gtggaagagaatgagttcactggcaactggtttctggccacaaaatctgtagacatgtcttgctgggaacagcagcgagaaccgca |
| | | agctaacggtctcatattttcctcacctgctgaaaagtgggcatatgcttgcaacccaatggctgataaaaacgaacaaaaaaaaa |
| | | tcaaagccgagtttcgagtaatggctcaatgcagtctggattttcatgttctctcgactctttggatgttaaaagtcgggcatctt |
| | | tttgatgccaaattatctacctgtgcttacggtaaccgcctgcgccgtactctagatggaaaagacatcaatgcactttcaattgg |
| | | ttcttttcaaccttacctcagaccttttcgtgattggcgtgacaatggcattaacgccatgcggagcgcgctaagtgaaagcaaaa |
| | | aaatcgtggcactcactgctgatgttagttctttctatcacgaactgaatcccgggtttatgcttgatccaaccttcgtcaaagat |
| | | attttggagttggaactcactgctgaacaaagcaagcttaatcgattattcattaatgcgttaaaagcatgggcaattgagactcc |
| | | gttgaagaaagggttaccagtaggtctccctgcttcagctgttgttgccaacgtagccctgatcgagctggatcgcgttattgagc |
| | | agcaagtcgcacctatatattacggacggtatgtagatgacatcattctggtcatggaaaatggtgcgaatttccgttccatggca |
| | | gagctatggcaatggttgttcgcccgttcttccggcaaactggactgggtaaagggcgaggaaaacaaacagatcagttttcaacc |
| | | aaactacctgcatgacagccagattcgttttgcaaatgcgaagaataaagtgtttatccttgcgggtgactccggaaaaaccttag |
| | | tggaagctattgctcatcagatttatgaacgagccagcgagtggcgagccatgcctcggttaccgcattcctcgaacaatgttgga |
| | | actgatttgcttgctgcaactcaaagtaatggcgaagtcgctgacaatttgcgtaaagcagatgcactgactatgcgtagggctgg |
| | | ttttgccatcaaactacgcgactttgaagcctatgagcgtgacctgcaaccgggcacatggaaaggccatcgccaggcattttttc |
| | | gggcatttattgatcatgttgtggtgctgccacaattctttgatttatcagtctacctaccccgagtgatccgactggccacggcc |
| | | tgtgaggactttgtcgaactgcgcaaacttatcttagcgctcgagaatatttgcgatgaagttcgagaaaattgcctccttaccat |
| | | caaggcgtgtcctgatgatcacctcccttttgaagcagagattattggcaaatggagggctcagctttttagcagtgtgcttgaag |
| | | ctatcgttgcggcatttcctccgcgtatttccaaggtgggtaagcaaacctggaatgaccatttaaaaaactggcacgcccggtgt |
| | | gggctagacattcaatattcgggtcgtgatttttcattaaagggctaccaagaacagcaggcgagattattctctttcgacttagc |
| | | gcacatgccattccgctttattggtctaccaaaagagatgattgctcaacggggcatacccgctccgaaaacagtagcccactgtg |
| | | cggaagcagcagaattactgcctgatattgtcgttttgggtaatcaggttgtagcaaaatggtgcaaatttaaaatcattccacat |
| | | ggactgctatttgccacccggcctttcagcctgccggaactctttatcctaaacaatgaggcttatacagcttcagctcagcaaga |
| | | aatgcgagctattattttcgctgttcgcggttttgtactcggtaataaaacaccttgtgtcgataaacaaggcatattgcaaatcc |
| | | ctgacggccaatctgctggaaaatatggggttgccatatctagctggaaaacgtccatgtcaagctggactgcggcggtcatgcgt |
| | | tcagccgatccggatgcaaaccgttacgctcgcttatgtcgcttgcttgatggtgtgatagcccaaccacataacagtcgttactt |
| | | aattctgccggagctctcactccctgcgcactggtttattagaattgcccgtaagttacaaggtcgcgggatttcacttgtcaccg |
| | | gcattgaatatttacatgccagtaaagcaagagtacgcaatcaggtatgggcttccttgtctcatgatggattgggttttccttca |
| | | ctaatgatttaccgtcaggacaaacaacgcccagcactgcatgaagagcaggaattacaacgaatagcagggctagaaatgaaacc |
| | | agaaaagaaatggacaacgcctcccatcattcaacacggtgattttcgtttttccttgttgatttgtagtgagctgaccaatatta |
| | | gttatcgcgcagcgctgcgtggcaacgttgacgcgctgtttgtgccagaatggaatcaggatactgaaactttcaatgccttggtc |
| | | gagtctgctgcgctagatatccatgcttacatcatccaatgcaatgaccgccagtatggcgatagccgcatccgaggccctttcaa |
| | | agatagctggaagcgtgatgtattgcgagtcaaaggtggtattacagattattgtgtaataggcgaaattgacgtacattctttac |
| | | gacaatttcaaagtagctatcgttctcctggtaaaccctttaagccggttccggatggatttgagatagagcactctcgaaaaatg |
| | | ttgccagaagcataagtaaaattggaaaaaaatatcgatgcaggttattaaagatgaggcaacatgccatagtcaatcataacctg |
| | | cagatgtaatttgaaactgcatgttgagaattacggatttatttgtgtattcaccctcgcataaaaatgaagtagctttcatattc |
| | | cacactactgataccccctgaaaatatataactaaaaaaaacaattttaaaacatgaggtaggaatagcaatctgactgtgatgta |
| | | gttatttttttgatgaagataattaggtgctcgttgttc (SEQ ID NO: 16) |
| 19 | pLG021 | ccactacaccggtgaccatgatttattgatcgttcctccttagtgaaccgattctgcccgcttaaccttaccccctggggggtaga |
| | | tgtaagcaacggagttctgttcgccgccaggtcaaaccacgatgacttgatcggcaggacagggaccacaatagaccttcaggtcg |
| | | gaatcagggatagaaggggacatgggcgaccgacagatatgaagatatgatggctatggcggcatctctgcccaccctcaggtcca |
| | | aagcgaaaggaatcggaatgccccgtatcaacgttgagaaactgctgcttgagatcgaaatcgacaaggtggcagagcgattgggt |
| | | atggcgcttaggagcgaatcagctacgcgcaagctcacgctgtgcccgttccatgacgataaaactccttcccttctaattgatac |
| | | gagcagagataattctggacagcattaccactgctttgcctgcggtgaacatggagatgcaatcgatctggtgaagggagttcttc |
| | | atatcgatttcaaaggtgcattagagtggctgtcaccaaactctactaccacccctgtaaatagggcgagaaaacagaaggctatg |
| | | cagcctgagcagccagaaggctcagggcttgcgcaagcttataagttatacctgttaagcaatgacaagcaacgactagctaactg |
| | | ggtgactgatcgcaagcttgatatttttttgatggaagatgcaggattcatatacgcacacaaaaactcactatctaaacaggttt |
| | | cctcaagaaaagattttggaacgaagcgtgaattagcagcaacattggaagaagcgaacctaatacgcaaaatccttccaagctcg |
| | | gggttccaaaactactatttaaatctacagtcaatccacgacaacaactatatagactttttttcaggggatcgaatcgtattccc |
| | | gataagagacgatcagaaaaaactactaggccttgccgcccgggcggtagatgagcaaccagcaaaatacctattctcaaaaaact |
| | | ttccaaaatccaaagctatttttagaatagagcaagctacaaccactctacgagcattggctaagcgaggcgaaacagatctacgc |
| | | ttatatatctgcgaaggattttttgacgctctaagattggaaagcttgggatttcctgcagtagcagtaatgggaacatcaattag |
| | | caaagaacaaattaagattatgaaagggcttagcgacacgctcccttcaaagctagcctctttgacaatctgtatttgttttgatc |
| | | gcgatgaagcgggattaagaggagcatccgaggctgtactaaaattcttaggcgctaatctcgacgtggtatttgtatggcctact |
| | | actgctcagcttacaagcgcagaccattcaaacacaagcataaaagatcctgacgaatatttgagaaatttgtccgcgccgcaggc |
| | | caagtcacttatcgatgtttccacctatggacctgtagtagcagtactagcaaatcagtttggtgtgcatgccgacgaactgcttg |
| | | aaaatctaaagtggaacagtgccagtcgctctcgaaaatacaggtcatttgagaaaactcgtgctgaactcaggaaagttgtagcc |
| | | aacccccatctccaatcaagcgacctttttttaaatggccgaacagatcttgactcggcggctcaaatagaatggattgatttttt |
| | | aagtgtcgacattgcgactgaagccgctccatcggaatgttatcttaccaactcaggcaccagactaaaccacgcccgactgctcg |
| | | cctatatgggctcacgaagaggagagttgccctgcgaagaatcaaaatgggagcggttagatattgcggcaagtgcattcaatgtg |
| | | ttgctcgctgaacgattggctaatgaaatacatggacccatcgacccgttcgaggccgtatgggtgccgaggtccttcggcgcaga |
| | | agagccgagattaaaggtgatgcctcaacctgaggatttaatagcgcatcagtacttactaaatgagctacttacagaacgctggg |
| | | atgcttccgctctcggtgttacagcattcagccagtgcataccagctgtccgctattaccgcgaagaaagaaaaactgttacgaca |
| | | ggaatatctaccccctcagataacacccaacctattatacttgaacagacgctaagtttcgcctatcaaattgatatggaggttat |
| | | tgagggcaggcagccagcttcagatcagggaatgtttcgtccgttcctagactgctggcgagactttatgcagtcccttaaaaatc |
| | | aagccaaatctataaattacgtgcatgttatccgcctcgatgtcagtcgatattacgaccgcatccgcagacacgtcgtaagagac |
| | | agcattcaaccatttatacaacaagctctggaaactgtcgctgataatgcaccggcgtttgctgaactgatgaaaatacaagcatc |
| | | tgcggatgaagcagcggacaaatccgcaataattgtcgagcaattatgcgacatgctctttggctacccataccttagccctgata |
| | | acgggagaattaataaatcagatcccttacgcggtattcctcaaggcccagtaatctcagcatggttaggctcagtggctttgttt |
| | | ccagtagatctcgcggcactggaaatgatgaacaaatacaatgtagacggggaaactcatctagggtatgcaaggtatgtagatga |
| | | catagttttactagctagcagctccgtacttcttgaggaactgagagagctagttgatcaaaaaactcggagcttagacctggcgt |
| | | tggtcgcgaaagctgacgctattccgccaatgtctgctgaggaatttgcagattatgcaaatcaagggcgagctttagaagcatct |
| | | ggtccagcgtgggaaccaccgttggctggcgatggtgaagcggggtgggagttttggtcaggcactcccccctcagatagacaatc |
| | | tgccctgcaactgctatcaaattgggagatatacaaaagcccaatagaaataatcttgcaaacagtgaaaacgtccttcctagcta |
| | | tggatttacgttctagcgagcttgcaaagggagcaaggctaatatggtacgttgtagcatccgacctcctctcagctgacattgat |
| | | ccaagcgatgcggcagatttagcgtgggaaatttatgatcgctattggaaggaatgtactgaggagtgtgggtggcagttaaaccc |
| | | ggatagtttcggatgggaggcaccgaatctgttcgcacttgagggactggaaaagcttatagatcataaaaatagcctccaatcgg |
| | | gtttaactgctttagaaaataccgttcggcacaaacgcatctctttcctagctagaaccgtgcttggggagcggttcaaactgcat |
| | | gctcttgaaagcagctctacgcttaagcaccagatagataaaagactagatctcctcgaatggaaagcgtcaaaatcgtgcggaat |
| | | gcccgttcgtagaactaaatcctacgcagagcgatcaatgtatattcgctcctggcaacccttcaactggttccatgccgcagtag |
| | | aagatttcatgctcgcggatcagtccagcggatccgacccattgagttcatatgtcactcagttccaatctatagaaaagagcatc |
| | | agacctaatcacgccgcttcttatgagttcttccggtatttactgccatccgatggcagcgatagcgatcttgagtttttctcaaa |
| | | aacagagaatcgatactccggcttagcaattcagattttggttgcattagtccctcgggaaagcataatacagattctctcaaata |
| | | gagcgcgcttactttgtcctctagaagctggtaaaaaactattagtcatgccccctcttcctggcgtcaatcagcaacgtatagtt |
| | | gcttgccagatcgatagctcctcagaaaacaaaatcaaaaaaatcagctcgtttgagtgctatgaaatagattcaactaaaaccaa |
| | | taccacatctctagacttttttggtgcaaactctgcgggcgtagttgtgcttacacccacatggaacaccgaagcccaacctcaat |
| | | ccgccatacttcgatcaaactcagaagtcccgaaaaatcttttgttggaggtatttgagaaaccgtcaaccggtttcccttccgct |
| | | attcagggattgaagcacgtagcctcactatatagagccattgtggtaataatggctgaatacgagaggcaaaatgatggtttaga |
| | | gcttatacccgcttggccataccttgccacagatatgacctctgggaactgctacctaatttgtgagggcgtaacgaaaggagaag |
| | | taggaaaccgagcatttgtaagagacggtgggcgggccctaagaaccattgagataccgatatacgaagcccagttgtggcgagcc |
| | | ggggttgcgctaagcgattacataggcctgcacgacgatattgctaaatttagctcctccgaatccgaaatacctttggatgcgac |
| | | aacgcttgccgccccgtcacagtacgtgctacgaagccaacttcgtaaactgaggggtgcctttgctaactcacaaatagggcggc |
| | | gcgttatgcccccaagttttcttccggcaagtgttgaacgtgcgcttgagttattggagcattttccggaagactcagatagtaca |
| | | aagatgcagctaatgcatctgcttgccactgaaaccgaaactgcgggaatgcgcgtccgctatgagaaaaatattgaggtcacaga |
| | | gctcacggtatttctacgtgcggtcgccgacagggttctaacgaaactacccttaagcataggtgaggtcattgctgcaccgacta |
| | | cagcagtcagtggcctgaggagagacctgagtggggtcttgacccttgccagaagcatatggtcgatggatgaagaagaaaaactc |
| | | tctccaatttttgcgtggaagatttttcgagctggaattgtaggtattggtatcgctgttgctctacgggggattatagcttcact |
| | | aagaagccacggggggtttgcacgctttgagggatttgattttccagcggaatgggagcttccccctgccacagcagttttatccg |
| | | aaccggcgacaacagataaaaccactgatgaaaatgtaagcctcctcgaccatttccgggtactcgtatcacatctcggacaccga |
| | | atgaggttggacgacaacggcgagccacaaatcccagaagaaatcagcacagaaataagaaaatacgctacagcattagcgggcct |
| | | cactactaaagactcaactgcggtggacgcaagcgactggcctttctttgatatcagcgaaaaagtttttgataccctaaatatag |
| | | aattattagagaacgtcagcaatctaatcaaaaacttagattccgcgcttggtctccaggtaattttggttacgcaacaatcatac |
| | | ggcttcaatgctcaaaccaaacgcttcactgactcaagaggacttgcatgggatataaagccatggatgatctcgcaatacccatt |
| | | gcgtgctcgccacgttgaggagtgttttgatcaagaccgtagaatcgtacgtgtatggagcgagatttacgaaaaaaacagtcaac |
| | | gcctgctttctatatcagtactaggcgagcctttcgcatcaattgcactatgtaaggacttggaatcgccttatgccgagactaaa |
| | | aatgtagacagcaagcacaacactgtattaggtcctagcgagcagggttctgaaagcgcacccatagatatttcaccgattcttga |
| | | aactgctgagcctgaggccgagactgccttagcagacacacaattaataccaaccccaaaccaaactagcactgaagacagctttg |
| | | ataaaatagatactgagcgtaatacaacacacaataaaaaactaccgcttaccgacgcaacactcaacgcccgaaagaattcattt |
| | | agaaatagccagctaacagcctggagcgataggaagtccaataaaaaccctgcccatgttcgggtagctctatttcagtgggacca |
| | | agagctgagctatgcacaccctatggtggaggccaccccacaaaaatggcctttcagttccgtctgtaaaccagcagttttaaaag |
| | | aacttaaacgcctatataactctccctatcaagcccttttgaatgcaactgaatctgccggtcaacaccacctatggaaaaacgaa |
| | | aatatttccctacccagctggggtgagcttcgtcgtcggcgattattgctcaacgcagtgaacgcatgccagtcatttggcgtgga |
| | | cttattgatacttcctgaatactcagtccgtgcagaaactgttaagtggttaaaagaagagtgcttacccggaaagacggtagcgg |
| | | ttttagcaggaacatttttagctttcgactccggtccgccccccctaaaacaaagcgcgagcctcaacctcttgtggcccgtaccg |
| | | cgtgatattgccgaatgcctcaaaccgcttgcacccaaaacaaatgaagatgctatgtccttgagtgacaagattgacaagggcat |
| | | tgtattgcaatggggcagatcaaagaaataccgatcagtagctctaaatgagttcatccggcctggaactgatcctctcacccccc |
| | | tgttcatgcccggaaaaataatagatgaattgagacgtgcaaattgggatctggacgctgatggtgttgttaagttgctagccaac |
| | | acagagttgccacttgcgaatttcatggagctgatatgctctgagattttcctgttcacgagcccaaccaacattccagagatggc |
| | | aagagattatgtttcaatgtgtgcaagatttggcttcggcgctgcagaagctcaagtctgggcggatctcaaactactatctaaat |
| | | ggctttcggtctgttccaagcctggtggtgccgactctagacgatcaattttgatcgtacctgccgcgaccactcgtactgctgat |
| | | tattggatagcaggccaagctggcttgcttgccgccggcactacaactgtatttatcaatggcgtaggatctgggcttaagggtgg |
| | | cagttgttttattggcagagagagctggaaaacaggggctggttctcacggttacattgagaccattacgccataccatggctggt |
| | | caaaaggaatttactataatagcaaacatgacccactgagcgaaattgatcaagcattggtgatcgcagatatcgatcctcataac |
| | | atgcttgaaggcaaacctagacctcagatgctgccagttcccttacagctagtggcatacctaccaatcgttgaaactgtcgacga |
| | | aacaagcttggaccaaactctctgtgacgcagttcaggttgaccataacaatattgcaagaattaatcagggtcagcgattgggtg |
| | | gacgacttaaaagtcgaaatgagttctggcaacttatcacgcaaagtataaataatgatgtcgacaacgactttatcattaacttc |
| | | agtaaatactttactgatgggaaagcgattcttgagcgagcaaactctttcttcaacaatggacaccaacagcctttttcatcggt |
| | | agttaagctagacctgctctgctctccggcactttacgactggctagaggccgatatgacgttgcgggagggtgaggcgttaccca |
| | | acatctcagtcccttcatggaccaaataacttcggatagattacgagcccctaggataaagcctgtcgataggggctggtcacatt |
| | | ccccgcagcagggcggtgccgataatagctgctcacatagcttagagagcagtcaccgcttggcactttggagctgggagagcgtt |
| | | ggcatcgtagaatcgtcggcagtgaaaattcggtacagctacggtacggcacctagcttctgtcaactaattcaaactacactcaa |
| | | caccatatactacggtgcctccagctatgccaacctacgttcagctaagaacgacttcactaggcatacatggtcgcccagcaact |
| | | cataatcccttggtcgcaggttcgagtcctgctgggcccaccaagctttgagagccgcgctttgcgcggctttttttgtgaagcca |
| | | agcactcagtttggtccgaacaccacgccaaagtgtttttcaagatcgcacatcccagaccacacgatgcacagacttcatgttga |
| | | agcgccgtcttcagaaataagctgggaaaaggtcaatagctttcaatttgtagcagccaaccgtgatcacaggtagagcacgggtc |
| | | gatttgatcttgcaatcctttgggcagcaagacccttgggctgttcaccggcgttgctgcacaaccagccacgctggaatcattac |
| | | tgtcatcaaggttgagaa (SEQ ID NO: 17) |
| 20 | pLG108 | |
| 21 | pLG023 | atccctgaattccccgaaggtgaacaatccactgttcacccttcaccgtatattaacccgttatcacactgaaattaaaagagaaa |
| | | aatgaaaggtgaacagtgtgaacaatcaaatcaaaaaaactttctactcccactatagcctgactggtcgtctccaaaacgagcgg |
| | | aaaagcatcaacaatgaatagttaactgttaactccgcgccaactcattaccacttaactcaatgatattaaatggaaaactatcg |
| | | aaatgaatactctgcaaaattaaatgcaaaaaaatatatgccagtcaaatttcgttacgcactctcttccaagaaagagataaatg |
| | | ctttatacgtccaccatactatgttatttttttaatacggctctgccttaaatctgtgaggttgtttcgcctcgaagtatcttatg |
| | | ttagcacatcacgctaccaatcagcggttagttacttgacgtaactgttaattggctaaagtttgcatagagtgattgggcggagc |
| | | cgtaaatttagtccataaatacagtaacgaggtagagagtgtctttacatgacaagctactgatgcttagtctcaattcggcgaat |
| | | aaagaagaagatgagacaatcccggagttacctaagttagagcctcagccctatcaagctggaaataagttgaaatgggataataa |
| | | agagctgaaaaatcagcccatcacttcaaagaatgacattaatgtaatatgcaaaaaaattgaaaacaaaagcattgtaattacat |
| | | cagcaaacgatgtagccaatctgttagaagtcccggtcggacaattattatttattttatataataaaaaagataactatagaact |
| | | tttgaaataaaaaagaaaaatggaaaaagtagaatcataaatgcacctcaaggcggtttatcaattctgcaagagaaattaaagcc |
| | | agttcttgagtacttttatcgccccaaaaaaccagcacatggatttattaaggataaaagtatattaacaaatgcagaaaaacata |
| | | caaagaaaaaatatgttgttaatgtagatttagaaaattattttggttcagtcactttcgctagagtatatgggatatttaaaagt |
| | | aagccatttaatttctctcatcctgcggcgagtatattagctcaactatgtactaaggatggaaaattacctcaaggagcatgtac |
| | | ctcccctgttctagcaaatttagcatcagcctcactcgataaacacctaacccaactggcacgtagaaaaaacatcacatatacaa |
| | | gatatgcagatgatattactttttcatttaatcaacgacaagtcagagaaatcataacgctagataatgaaaataattttgaattg |
| | | ggcgaggcgattatctctgtgatagagaaaagtggcttcagcataaacacaagtaaattcagagttcagaaaagaaatgaacgtca |
| | | aaaagttactggtctagtggtaaatgaaaaagtaaatgttgagcgtaaatatcttagagttactcgttcattagttcataaatgga |
| | | gagaagacaagttaacatcagcattgttgtttgttactaaaaaaggttttaaggcaacaaataacgaacatgctatatcaattttt |
| | | cgcaatcatatttatgggcgattgagttttataaaaatgatccgtggtgaggacttcccgttatatcttaaattaatggctgaaat |
| | | gagtcatcatgatcctttaaaaacaaaagaagggcttagagcaatgaaagaaactgaaacttacgatgtatttatttgtcatgcaag |
| | | cgaagataaaacatccatcgcaattccaatttacgaagaattaattaaattaaatatatcaacattcatagatcatgttgaaataaa |
| | | ttggggcgattcattaatccaaaaaattaactcagctcttgtaaagtctaaatatgtaattgccattctttcggctaattctgtag |
| | | ataaacattggcctaagaaagaattgcattctgtgcttgcaagagaaatcactgaaggtgaagtaaaattacttactcttgtaaaa |
| | | gaagcagatgaagcaatagttgctgaatctttgccgctcttaagtgataagctttatatgacctataaagataatccggcagaagt |
| | | tgcagataaggttcgtgcgcttttaaacaagtgacagctactgtcaaatgtgtataaagtcattgatattttatataaaatcaatg |
| | | gattgcaatccatataagattccttatgcatcagtgacccggtgctcgcccggtcactgcttcagtcccagcagaactcagacgag |
| | | gcgcttaacatctaacgggatgccaacccgacgtttggttttatcggctatctagcctatatagaagca (SEQ ID NO: 18) |
| 22 | pLG024 | ctattgtgagcgagaaacgcgctactactatatatagacagacaagatgcacttactgaataaatactcataacggagaaaccagc |
| | | tgtatagtgaacaatagatttccagtagcatatttttacttcacttttagttattaatatgataatcataaactacggctctgcct |
| | | taaatttgtgaggttgtttcgcctcgaaggaactaatgttaggacatacgccaccgttcagtcgatggtaacgcttcttaactagt |
| | | ggtccgctaagtgatgcgcaaagtgattgggcagagccgaaacgtttacaatccgataggagttggttttgtcgctacatgataaa |
| | | ttattaatgcataacttcgcattagccaataaaaaaagccctgacttcatatctgaacttcctcaaattgaacctaaaccatacag |
| | | caatggacataaaattaaatggataaaccacacacttactagcactgaagttactccccctgataacctgattaaaatatgcatat |
| | | tgattgagtcaggggaaattgctataacatcagtaagtgatattgccaatttacttggagttcctgctggccaattactttatata |
| | | ctatatcgtaaaaaagataattatcgtacttttgaaatagaaaagaagaatggtaaaaaaagagtcattaatgctccttgtggcgg |
| | | tctatcgatactccaaacgagactaaagcccgttcttgaatatttctacaggccaaagaaatctgctcatggttttataaaaggaa |
| | | agagcatcattactaatgctgggatgcatattaaaaaaaattttgtcgtaaacattgatctagaaaactatttcgaatcaataagt |
| | | tttgctagggtttatggaatatttaaaagtaaaccttttaattttgctcatcctgcagctactgttttagctcagttatgtactca |
| | | caatggaaaattacctcaaggtgcgtgtacatcgccaatattagcaaatattgcatcagcttctctagacaaacagctcacccaat |
| | | ttgcaggaagaaaaaaaatatcttattctaggtatgctgacgacataactttttctttcaatcagagaaatattgatataatcaaa |
| | | aaaaacgacgacggaagttatagtcttagtgaaactatagacaatattatttcaaaaaatggctttaaaataaattatgataaatt |
| | | tagagttcaaaccagaaatacaagacaaagtgttactggcttagtggttaatgataaagttaacattaacagaagatatataagaa |
| | | ttacacgttcaatgattcatagatggacagatgataagctaaagtatgcacttctctttgctacagaaaaaggatatcaggcaaag |
| | | gataataaccacgcaattcaaattttccgaaatcatatttatggaaggcttagctttataaaaatggttagagggaaagactatcc |
| | | aggatatttaaaactgatgtcatacatgagtcataacgatccattaaaaacccaagaaggattgcgagcaatgaaagaaacagaaa |
| | | actttgatgtttttatatgccatgcaagcgaagacaaaaaagacattgcaattccaatatatgacgagttaactaaacttaaaatt |
| | | tcagccttcatagatcatgttgagataaaatggggcgactccttaattgataaaataaatgcagcactagttaaatcaaaatatgt |
| | | catcgctattttatctgctaattcagtcaataaggaatggcctcaaaaagaattaagagcagttttagccagcgaaatatcgagtg |
| | | gcgacgtaaaacttttgaccttattaaaaaaagaagacgaggaggtcgtaaacctatcattacctttacttagtgataagttttat |
| | | atggtctatgataataatcctgaagtagtcgccaacaatattaaatcactcttacaacgataattctctcacaaaagaaaatgtgc |
| | | agattgatgcgtattaagtattaatctgcacatacaaaaaaaataataaaataatacatttttcataacttgtaggtaacaacaat |
| | | atatgtcgtaacgaatatttggataacctctataccctattaaccaaccaattaactctatgtaatctcgcagcc |
| | | (SEQ ID NO: 19) |
| 23 | pLG025 | cacgtaaatatgaaaactgttagcccacatagcccaacaaaaatatttgatagttaaccttctgttactaaagaaaacaggaaagt |
| | | aaaagtgggctaaagcttatgcgccctcgatgttgggctagccccaaaaacggtaaatttagcttaagtgcataattggttagctc |
| | | aaaagcattatttttcatttaaataaattagttaattggtcttgtttagatgattcaactgggctgactactttctttgtatatac |
| | | tccggataaattttcccagctaacttgcctaatcatcactctgatgccagaaatgaacagaacgcaaaccatctataacttattga |
| | | ggattttgaaaaaaattgattgggggcttgagttatatgatgactatgctaatttaatacggcacatgcaggtagatttgttggtt |
| | | gtggtatcgcaatcagtgttaacaaggtcgggagtattcgccctctgactgccgtcaagtcatcttggcgtcaccgttaaatgcgt |
| | | aagagtacctgcatgtgcattaacataatcaataatggaatttactgttatgtttaaacctacctatctggcaaggctgcaggctt |
| | | gttgtaacaaatttgaactggctgatttgcttcagattaaagttacatttctgactaatgttttgtatagaataaggccagaaaat |
| | | caatacaaaaaatttactataaagaaaaagtctggaggagagcgggagatctttgctcctgatgaaaaactgaaagatattcaaca |
| | | acgactttctgaacttctatatatatgccaggaagaaatttgggcaaaaaataatattaaacaaaatgtatcacatggttttgaga |
| | | agaataaaactataattacaaatgctgagaggcatcgagataaaaatattgtatttaatattgatattgagaatttcttcccatcc |
| | | tttaattttggtcgcgtgcgaggatattttattgcaaaccaaaatttcaagttacatccaaatgttgcaaccattattgcgcagat |
| | | agcctgcctggatggatcgcttccgcaaggaagcccttgttctccagtaataactaatcttatttgtaggattttagatttcagat |
| | | tatcaaagctagcagtcacatatggttgtagttacagccgctatgcagatgacattacgttttcaacaaacaaaaaaaacatccct |
| | | gatgcattagtttctaatgagaaagaaaacgaaccaggtaagatattggtagaagaaattcatcgtgcaggcttcactttaaacca |
| | | taataaaaacagagtgtctaggtgtacatcaagacagcaagttacaggtttaactgtaaataaaaaaataaatgtaagcagagagt |
| | | atataaagaatacaagagcgatggcgcattctttatactttgaaggttcgtatacacttattgagaaagatggaaaacatagaaag |
| | | ggcacccttagtgaattagaagggcgatttgcatttatcgatatgcttgataaatataataatgtggaagcaaagaaaaatgcgcg |
| | | tcctgagagatatgtggttaaaggatttgggttggattttaagcagagacttaactccagagagaaagcatacagcaaattcctat |
| | | actataaaaatttctatggaaatgagcaaataacaatcttaacagaagggaaaactgacccggtttatcttaagtgtgcaattgat |
| | | tctttgtttttggattaccctcagttagttagagaggaaaaaaacacaaagaatagagtgttaaaagttaatttatttaaaaccaa |
| | | tgacaagaaaaaatattttctcgatttgtctggtggagctgcagactattcgaggtttttcagacgacatggtttactttgtaaag |
| | | cgtatgaaaaacagcctcctaaaaatccagtgataattttattagataatgacacagggccatctgacttcataaatcaaataata |
| | | aaggattattcgcatctaccaaaaaaagcggaggatgttagaaaaggggcgttttatcacttagagagtaatttatatgttctttt |
| | | tactccgttattaccaggggataactattcttcactagaggatttttttgaaccaaaagttttgcaaatgaagtataatggaaaaa |
| | | gcttcgataaaagcaataatcatgacagttctactacatttggaaaagatagatttgctacttatatagtaagggaaaatagaaaa |
| | | actatcgatttttcattattcaaacccatacttgattcaattattgaaatcaaaaaacattttatcaatctacacccatcaaagtg |
| | | atggttatgaaaagagataaaaatgctgatgtcaaaagaggcttatgctcggcacagtggagtgagctgccaaactgtcgatgact |
| | | gggtagccggtggggcggaagtagttatgtcccgtagcaaggttaagatttgctcttgtgtgtggggaaccttagtcaattacttt |
| | | cctggcgcactgtgttagattttgtaaaattttaaaagactaaagatttaatatcacttctccatggaggttgtg |
| | | (SEQ ID NO: 20) |
| 24 | pLG026 | ctatacgccgttatagctgaattttccggtgatttcagggcacattaaccaatttagataatactatagtaatggttgggctgatt |
| | | tttcaagaacaaaagtaattttcaagctttgtaacatgttgattttccgcttttcgctcaagcgagctttcatctttgcaagccca |
| | | tatgttcgtttttcaagcgattattcagatacgttaacttcccatggcagtgcatgactatgctgcatgaaatcgcatgatcgatc |
| | | gaggatcgtctatgcttagaccagccagaaatggcgggcttttgctcatgtcatgcagctgcatgaaaaccactgcataaagtggg |
| | | caggcgtggcggggatacgagggcgcgctatcacgtaaaataggcaaaatacttctggaaaacagaaagttgaagtgatatgttca |
| | | taaacacgcatgtaggcagatttgttggttgtgaatcgcaaccagtggccttaatggcaggaggaatcgcctccctaaaatccttg |
| | | attcagagctatacggcaggtgtgctgtgcgaaggagtgcctgcatgcgtttctccttggccttttttcctctgggatgaagaaga |
| | | aatgacaaaaacatctaaacttgacgcacttagggctgctacttcacgtgaagacttggctaaaattttagatgttaagttggtat |
| | | ttttaactaacgttctatatagaatcggctcggataatcaatacactcaatttacaataccgaagaaaggaaaaggggtaaggact |
| | | atttctgcacctacagaccggttgaaggacatccaacgaagaatatgtgacttactttctgattgtagagatgagatctttgctat |
| | | aaggaaaattagtaacaactattcctttggttttgagaggggaaaatcaataatcctaaatgcttataagcatagaggcaaacaaa |
| | | taatattaaatatagatcttaaggatttttttgaaagctttaatttcggacgagttagaggatattttctttccaatcaggatttt |
| | | ttattaaatcctgtggtggcaacgacacttgcaaaagctgcatgctataatggaaccctcccccagggaagtccatgttctcctat |
| | | tatctcaaatctaatttgcaatattatggatatgagattagctaaactggctaaaaaatatggatgtacttatagcagatatgctg |
| | | atgatataacaatttctacaaataaaaatacatttccgttagaaatggctactgtgcaacctgaaggggttgttttgggaaaagtt |
| | | ttggtaaaagaaatagaaaactctggattcgaaataaatgattcaaagactaggcttacgtataagacatcaaggcaagaagtaac |
| | | gggacttacagttaacagaatcgttaatattgatagatgttattataaaaaaactcgggcgttggcacatgctttgtatcgtacag |
| | | gtgaatataaagtgccagatgaaaatggtgttttagtttcaggaggtctggataaacttgaggggatgtttggttttattgatcaa |
| | | gttgataagtttaacaatataaagaaaaaactgaacaagcaacctgatagatatgtattgactaatgcgactttgcatggttttaa |
| | | attaaagttgaatgcgcgagaaaaagcatatagtaaatttatttactataaattttttcatggcaacacctgtcctacgataatta |
| | | cagaagggaagactgatcggatatatttgaaggctgctttgcattctttggagacatcatatcctgagttgtttagagaaaaaaca |
| | | gatagtaaaaagaaagaaataaatcttaatatatttaaatctaatgaaaagaccaaatattttttagatctttctgggggaactgc |
| | | agatctgaaaaaatttgtagagcgttataaaaataattatgcttcttattatggttctgttccaaaacagccagtgattatggttc |
| | | ttgataatgatacaggtccaagcgatttacttaattttctgcgcaataaagttaaaagctgcccagacgatgtaactgaaatgaga |
| | | aagatgaaatatattcatgttttctataatttatatatagttctcacaccattgagtccttccggcgaacaaacttcaatggagga |
| | | tcttttccctaaagatattttagatatcaagattgatggtaagaaattcaacaaaaataatgatggagactcaaaaacggaatatg |
| | | ggaagcatattttttccatgagggttgttagagataaaaagcggaaaatagattttaaggcattttgttgtatttttgatgctata |
| | | aaagatataaaggaacattataaattaatgttaaatagctaatgaacagccctaacgttatgaacgctaaggctgatttttcg |
| | | (SEQ ID NO: 21) |
| 25 | pLG027 | aattccccgaaaatccgcccgtttttactgaaaaaagccatgcatcgataaggtgcatggctttgcatgcgttttcctgcctcatt |
| | | ttctgcagaccgcgccattcccggcgcggcctgagcgtgtcagtgcaactgcattaaaactgccccgcaaagcgggcgggcgaggc |
| | | ggggaaagcactgcgcgcaagctatgtgaggtgatgtgtaatacatatcacgaatagcgtaggtagctgttggctttgcctgatca |
| | | aggtgacagtatacatatcttaaaatataaatatttatgattatttatttgaaagaggttgaataatgatttttgatgaaaaaaga |
| | | catttatatgaagctctgctgcggcataattattttccgaatcagaaggggacgatttcagaaatcccaccatgtttttcttcaag |
| | | aacttttacaccagaaatttgtgaattaatagtttctaatgagccggggaaaagaaaattacatggatacgattgtgtcgaatact |
| | | catcgactaggtataataactttcccagagtattatccttaattcacccaagagcatatgcacagttagcaaagcatttgtatgag |
| | | tcttgggatgagattcgaaaaatcaaagaaaataaaaacagtatgattaaacctgaaatgcatcctgacggtagactttttatcat |
| | | gaattatgaggatgcagaaacaagaactgtaagggagttaaacgatggatttggaagacgatttaaagttaaaactgatatcgcag |
| | | gatgttttaacaatatatattcacactcaattccttgggctgttgtcggtgtgaataaggcaaagacatcaatgaataagcataaa |
| | | aatagccaagatgttcattggagtgatagattggattattatcaaagacaaacaagacgaggcgaaactcatggtgtccctgttgg |
| | | acctgcaacgtcaagtattgtatgtgagataatattaagttccatagataatattcttgagaataaaggattcttattcagacgtt |
| | | acattgatgattatacatgttattgtaaaactcatgatgaagcgaaagagtttctccatgttttaggtactgaactttctaagtta |
| | | aagttatctctaaatttgcataaaactaaaattaccagtcttcccagtacattgaatgatgattgggtgtcgttgcttagtattaa |
| | | ctctccatccaggagagtattcaggaataatgactcggatatattatctgcatctgaggttataagctttttggattatgcggtac |
| | | aacttcatctgacgaatgggggcggtagtatattaaagtatgctatatctttaattattaataaagtagatgaggcgtcagcaaga |
| | | gagatgtacgactacgttttaaatctgagttggcactatcctatattaattccatatttagatgtattgcatccaaagattaacat |
| | | taatgatgaggtcaggttaaaacttaatgaggttttgaattcctgcatagataataagttttctgatggcatggcttgggtgttgt |
| | | attattgcttaaaatattccattgatattgacagttgtctcattagtaagatttttgaaaacggtgattgcctaagtatttgtatt |
| | | ttggataaaactggaagatatgataaggaaatagaagaattttctaaaaatataatttcattggattatttgtatgaggttgataa |
| | | atattggatattgttttatcagcgattctattcagggaaaggatataatccttacaatgatgattgttgtttcgatataatgaaaa |
| | | catatggagttaattttatgcctgatgatggttatcaaacgaaagctgaacactattgtaatatagtaaatagtccatttcttgag |
| | | aatgatgaacaagtaataagttttaacgattattgttcataatttataattagcctccg (SEQ ID NO: 22) |
| 26 | pLG028 | cctgtcaaaaaatccccgtaaatcccgctatttttaacgaaataagccatgcatccataaggtgcatggttttgcatgcgttttcc |
| | | cgttcctgtactcccgaccagcgtcagtcccggcgcgacctgaggtcacctttgcacctgcattaaaagcggccccttaagcgggc |
| | | aggcgtggcggggagagcattgcgcgccaaagcgtattgatatactgccagcattttttgatactcacacccatctacaggagtag |
| | | gtcactaccgatgtagagcttttccggattcagataaaaccacttagcatcggagcaaagtaactcaataccgaacaataaatatg |
| | | agcccttcgtgaaaccgggtaaggtcaaactcataaaccaacaaaaggggaaaagtgggatatgtgaggcgtgtatgatttttatt |
| | | tattgggcttcgttaaaaatggtgatttaatagccctttaaatttatcactttttaactaactccgagggtttatggttatttttg |
| | | atgaaaaacggcatttgtatgaagccttactgaggcacaattatttccctaaccaaaaaggttcaataagtgaaatacctccgtgc |
| | | ttttcttccagaacattcacaccggaaatagcagagctaatttcatctgatacatcagggcgcaggagtctacaaggttatgattg |
| | | cgtggaatattacgccaccagatataataacttcccaagaacgctgtcaatcatccatccaaaagcgtactcaaagctagccaagc |
| | | atatacatgataactgggaggaaatacggtttataaaagaaaatgaaaacagcatgatcaaaccagacatgcatgctgacggtcgc |
| | | atcataatcatgaattatgaggacgcagaaactaaaaccataagagagctaaatgatggttttggacggcgatttaaagttaacgc |
| | | agatatatcaggctgctttacaaatatctactcacactctatcccgtgggcagttataggggttaataatgcaaaaatagccttaa |
| | | atactaaagtaaaaaaccaggataaacattggagcgacaaacttgactactttcagcgtcaagctaaaagaaatgaaacacatggt |
| | | gttcctattggtcctgcaacctcaagcattgtttgtgagattattttaagtgctgtggataagcgtcttagggatgatggattttta |
| | | tttagacgttatatagatgattacacatgctattgcaaaacacacgatgatgctaaggagtttttacatttactcggtatggagttg |
| | | tctaagtataagttatcactgaacttacataaaactaaaataactaatctcccaggaactttgaatgataactgggtttctttgct |
| | | taatgtaaattcaccaacaaaaaaacgttttacagatcaggatttaaacaagctaagttcttctgaagtaattaatttcctagatt |
| | | acgctgtacaattgaacactcaggttggtggtggaagcatactaaaatatgctatttccttggttataaataatttagatgagtat |
| | | acaatcactcaggtgtatgactaccttctaaacttatcatggcattatccaatgctcatcccatatctaggcgtacttatcgaaca |
| | | tgtctatttagatgatggtgatgaatataaaaataaattcaatgaaattttgagtatgtgtgcagagaataaatgttctgacggca |
| | | tggcctggactctttatttttgcatcaagaataacattgatattgatgatgatgttatagaaaagattatatgtttcggcgactgc |
| | | ttgagcttatgcttgctagatagctcagatatatatgaagaaaaaattaataattttgttagcgatatcatcaaactagattatga |
| | | atatgacattgacagatattggctccttttttatcagcggttctttaaagataaagccccaagcccttataatgacaaatgctttg |
| | | atattatgaaaggttatggcgttgactttatgccagatgaaaattacaaaactaaagctgagtcatattgtcatgtcgtcaataac |
| | | ccatttctagaagacggagatgagattgtaagctttaatgattatatggcgatagcgtagcttttaggcctcatt |
| | | (SEQ ID NO: 23) |
| 27 | pLG029 | gcgttgaatggtataactatggcacggttaccgcatgttttgagctgtaatcgaagttatgaaaattgctatataaagcggtcgct |
| | | gttgtggagatacgattgcgggaagtgatggaaagagctataaaaagtacagaggatagtttaatgagggtattatgaaccgtcag |
| | | ccgtttacttcagcagcacttaaacgaaacttaagtgaaagtgagaaggcttattattttaaaaaaaataatgttgctgagttaga |
| | | atcattaattagtgatgccgttttaattgctaatgagaattttcgctctggtgtgagtgtaaagaaactaaatattaagggacgct |
| | | gcgtttacactgcttcatgtttgaaggaaaaaataatacttagacattgcaatgcaaatttaaaatgccttgaatcgcttcgtccc |
| | | aaacaacgaaatacaataattagtgagcttaaaatttatttggaagaaggtactccattcaaaatatatcgtttggatataaagtc |
| | | tttctttgaatcaattgatttaccgcagctttttcagctcttacataacgaaacacgactgtctagacatacaaaaaatttgctag |
| | | aatggtatcttaaatcgtgtgaaaggcttcactcttcgaaaggattacctagagggttagaaattagtcctatgttatcagaattg |
| | | tacttggcacaatttgataatagtattcataggcatccagaagtattttattattcaagatttgtagatgatatggtaatcgtttc |
| | | aagtggttgtgaatgtgaagcgtcctttatggaatttatacaagatgtattaccaaagggattggcwaaataaaaataaattaaaa |
| | | atatctccatgcataccaaagagaagtaagggtttaaataaacaggataaattgcttcatgaatttgactttctagggtactcgtt |
| | | ttctataatagacacacctttgagcaaagatggtgagattaatagctgttacagaaaggttgttgttaatttatctaaatctcgcc |
| | | tgaagaaaattaaaacaagaatagctaggtctttctactcttatcatattaatggtgattttaaactattgctagacaggatttct |
| | | tttttgactagtaacagggatttaaatcgcaaaataaaatcgttaagttctttagaaaaaagcaagataagtacaggtatttatta |
| | | cagtaatgcgaagttagatgttgactccatatccctaaaaaaattagatgactttttgctatattgtgtgcaatctaatactgggc |
| | | gtttgaatagtgttgcaaaaaaaccttttaatttgaagcaaaaaaaagaactgctaagaaatagttttagaaaaggctttgtggat |
| | | agagtatatagaaagtataactttaagcgctatactgagattacaaaaatatggttataaagaaaaacattaaacttgataagaaa |
| | | gattatctcagggctttactatgtgatacactgcccggtgattgtccaattattttttcaaatgatggcttatatataaacttaac |
| | | agaatatgatagagtttgtaatgatttgttacattttactccggtttcttctttcttaaaaaaaatagttaaccctaatttagact |
| | | cttctattagtgtcgcagatcgccaccgagaaaagaagaaacaaagctccccatttggctattgtatagtaaaagatgcctttagc |
| | | caaagacatctttctttaattcacccaagatctcaaattaattattcggaattttataaaacatactcatccgttatcacattaaa |
| | | tactttaaaaagtaatttttctattcgctacccacgtaaggtcgctaactctttctttttatatgaaaataatgctttggaaaaat |
| | | ataaaggggaagatatcgaaacaacaaaggatgagttaatgaggaaatattcatcctcttattttagttatggcggtttcaacagg |
| | | atatataaactatttcaaagtaagatgtttattgagcttgagaaaagattctcggtgatgtggatgttagatgtatcacattgttt |
| | | tgatagcatatatacgcattcggtttcttgggcattaaaaaataaatcatatatcaaaaaacatgttaaacacagcaatcaatttg |
| | | gacaagaattagatacactgatgcaacgtagcaataataatgaaacaaatggaatacctattggttcagagtttagcagggttttt |
| | | gcagaattaatatttcagcgaattgattgcaatattgagtcatgccttcttagtgaacatggatgggttaataataaagattatgt |
| | | tatattgagatatgtagatgattttattgttttttgtaatggtgagtcaagtgccgaagttattacaaaaataattaatgtgaagt |
| | | taaatgaatataatctacaattaaatgtaaacaagcttaagaagtattctaggccattttgcactagcaagacaagtttgattgtc |
| | | aaagttaatgaattaattcgcaatttagaaattaaactgtatgaaaaacgtgatagtggctttactttaaataaaataagaagtaa |
| | | gcatgatttaaagatatatgtaattaatcatgtcaagtctatatgcattgaaaatcaagtgtcttattctgatgtttcatcatata |
| | | taatatcatctctttccaaaagattaatatcaataattgatatattacgagttcaagaaaatgaagatgatgtagatgtaaaaaaa |
| | | aggattaaggacttaattttcacaataaccgatattatgttgttctttttcagtgttaacccaactgtttcatcatcttataaatt |
| | | atcaaagacaatggttgttgttaataactatttgaatgaaatatctagtgactatagtagtatttttatgactacgttagtgaatg |
| | | ctgcggaaaacattaattttggtgagaatgataatgggctgtttattgatgatttcatttcaattgaaaaggttaatttaatcttg |
| | | gctgctactttttttggagataattatcttataagtgacagtttttttcatggagttatacataaaaagaaattggactactttac |
| | | tataatctcactgctattctattttagaaacagaagatcattccgaaaattgaagtgtataatagagggtgaaataaaggaaatat |
| | | taagttctaatatggatttgctgcaatcatcggaaaaggcacatttatttttggatgtcatgtcatgtccatttgtctcaatagag |
| | | acaaggcgttttttatatagaaaatatctcaagagctatgagccaaagctgaacagaagtcatctggagattgagaatgatttgca |
| | | atctctgcttcaaacatattggtttgtcaagtgggatgagttagatattgtgaaaatgattgagaaaaaagaattgaaagaaagct |
| | | attaatttgataaatatgagtcgtggtcagtttcaaaatacttacgtcatcgtcgtcggtgtattttatatcgattatgaagacga |
| | | tttcgctggaactgaaatcggcttgaatgcttaaacttaagctaaaaaaacagtttgagaccaaagcctaaattattaggctttgg |
| | | attttcaggttcagttgagagtaattgctgtctg (SEQ ID NO: 24) |
| 28 | pLG030 | cttgagtttgcgtaagataatttcgtgaaaattaaagcaattaatataaaaaatgtaattactagtgtgtacagatatgaaaaatg |
| | | atagttataaaaccatatgaaaattgaagaaagagttcaatttttgccttgtcagtaacaaataggtagcttattgaaaaaagata |
| | | aaaaattaacaaaaaatcaataaattcatatagaataaaaatattaaagaaatgaaataagtgtttgcttcatcagttttagggat |
| | | acattaaagtggttgataaagaaaaatattatactggattaataaaagatataaaaatagtagcttatgcaagattcaataaaata |
| | | cgtcgtttaaagagaaataattttttaggattgttatctatttcggtagtttctatcttagttattatattatcaattgtagaaaa |
| | | aatttataatataaaaacaatgagtttaattccattgtttgaaccaaatatagaaatatggttcttttgtatacttgcttcaataa |
| | | ttattctttgtatatctattgcactctctactatgaagattgatattgaaatagaaaggttaaataaaagtgcagttgaacttaat |
| | | gaagtaaggcggaaaattgaatttaatattgagaatagtaattatcaaaatagtacattgtttgataaatatcttgaaataataaa |
| | | gtcagacttaataaatcatgatgaggttgattataaaataaataagtatttagtcagtaaagttggtagtaagtttgcttattatc |
| | | gaatgtattttattgatcagaattttacatcaatattttatctttttataacatttttaagcttttcttcaattatttcaattatt |
| | | ttgcaggtaatgttgaagtgataagacaagattttagtgtaaattccctgttgagaatcacaactaaaaatgaaattgttaaattt |
| | | aacttgggtcgtaataaggaagagtatgctattgcattatctcaagtttctaattatctattagagggcaatgaaataatagataa |
| | | tttaagctgtagaatagaaagaaataaagttatatttagtactaattcaattaatactttttatgctttaaaaaaaatttctaaag |
| | | atttaagccgattgtataaaattgagcctcctaatagagatgatatttctgaacaaatttatagaatttttgaacactctacaagc |
| | | tatagtattgtaaggttagacattaaaagtttttatgaaaatattcaatataatgaggtaattaaaaagctggatagagataaaat |
| | | actagttgcaaaatctattaaaattcttaaggatttatataactttattgataatggtttaccacgaggtttatctataagtccta |
| | | ttttgtcagaaatatttatgaaagaagtcgatcaacaaattagaaatatagatcatgtatactattatgctagatatgttgatgac |
| | | ataatagtaatttcaacagataagagtgattctatatatgaaaaaacaattaaagttttagagaaatatgatttaaatgttaatag |
| | | taagagatatataaaaaatattcctgctgtgaacaataatgaaatctcaactttatataagtttgattacttaggatataagtata |
| | | ttatagatacaatttcatataaaaataaacgaatagttaaagcggaactgtcagatgataaaaaaagaaaaattaaaactagaata |
| | | atacatagtcttttagatagagtttataatacaacgcattatgatcgggaggagttgttaattaagcgattaaaagtgttatcctc |
| | | taactactcaataacatataatgaattgtcaaaaactaatttaaaagctggtatgttttatagtcataggttagtaaataattatg |
| | | gtatttttagtgaatttaataaatttttatctaaagctatctactgtcaacaaaacaatttctttggtaaagctatgtcgcagatt |
| | | cctagtaaagaaaaagaaaatattattaaaagtatttgttttgttagtggatttaaagataaaaactttattgagttagagagggt |
| | | tgaaatggaacgagtaaaaaagtgttggaaaaataaacgatataagaagctttgaggtaaaaatgaaaagtaagatttatttagat |
| | | aaaaaggatttttatagagtattgttaactgatgtattaccctatgaagtaccttttattttaagtaatgaaggtttttatagaaa |
| | | cttaaaaagcaactcatttcattcagttactaaaaaaatattagaattaactttatttacttcacaagtaaacactaatcctttta |
| | | attttaaaatctctaaagatgatagtaattttaggaagttatatttagttcacccaagttcacaaataaaaatatcaaatttatat |
| | | aaaaattattatcaattaattacgcatttgtgtagtagaagttctttttcacttagatatccaacttatgttgcaaaagcttttta |
| | | tagtatagaaagagatagatctaattccgaaaattataaagatgaagatattgaattactgtcacaaaaaagccctaaatatgcaa |
| | | gtacttattttgtatataaagatatcagttttttatataaattctatgattcttatagatttcaccgtattgaaaaaaagtttaat |
| | | aaactattaaagtttgatattgctaaatgttttgactcaatatcaacatttcaattacctagatcagttaataaaaattgtagctt |
| | | tgaaagtcatacagatatacatagttttgaacatttattttcttcaattatgaaaggtgcttatcatggtaatacacatggtattg |
| | | taataggaccagagttttctagaattttcgctgaaattttattgcaatctatagatgtagcaataaaaaataagttaagaaatgaa |
| | | atgggaattaaggagggtgttgattatgttataaaaagatatgtagatgattattttttattttataataatgagcaaacttcaaa |
| | | tttaatttttgaatgtattgttgaagaactttctaagtatagactattttgcaatgaatcaaaaagtattaggactactattcctt |
| | | ttattacaggtattactattgctaaacatgaaataaggaagagattagaaactttttttgaattatttgagtcaataaataataaa |
| | | gatgattatattgggctaaaattaaatcattattataaaatatcaaatcaattaattagtgatattaagtgtattgtttttaataa |
| | | taatgtaagttattcaagtatttctggttatttttttactttaatgaaaaatcatgttttgcatataaaaaatagtttttcttttg |
| | | aggataaatctaaagttgaaaatttaagtaagttatttcttattattcttgatgtttcgttttttgtttactgtatgaattttaaa |
| | | gttagaagcacatatttaatttctcaaattatagttttgattagtactattgctgaatcatttgatttaaatttgatagatttaat |
| | | taataaaaaaatatatgatgaggtggatttggttttaaagataaagtcaaattcaaacttattgaataatattgaaattttaaatc |
| | | tattaattgctgttagagatattgatcttaattatcagatcttagtagatgatcttatgttattgttttcttcagaaaggattaat |
| | | aagtataattatttctctttaatgacttttttattttatgttcaaaggaaaaaacagtatcagcctatcagagatagaatttatgc |
| | | aataataattcaaaaatttaatcagaataatctaaatgtctcaaatgattctgagttaattcacattttttttgactcacttagct |
| | | gtccttatttaactaaaaatcaaaaaattaatataactaactctgcattaaattctattattaaattaaatgataatgaaattgat |
| | | gtttttgtagaagaaatgagcaaaactaattggtttattgactggaacttgcaaacaaaagatgcaattcagcgtttgctgatgaa |
| | | aaaagaattgaaatcaccctatgaaaattgagataattaagctagaaactagatatacctccgacatttgttggttgattttacac |
| | | actatataactcctagtttctataaaaggatgtttctaacatccttttattttttttgagatttaatttttcttttagtgacaact |
| | | aagttttactataactaatagc (SEQ ID NO: 25) |
| 29 | pLG031 | actgctcgacaaaacgaaccgttcattcgcgaggatggtggcagtgaatgaggtggtcagttttatcagcgcttcaaggtagcttt |
| | | ataggatggattgtagcgaagtgcccaacaaattgattgaagctaagggcattgagcattgcatgcatcatgctcagactgacaaa |
| | | aaatcaaaataaatggattgatacggacatgacagacagcgtacagactgaaactaccgagggaaaaatcatcatcaacttgtttg |
| | | ctcccaatcttcccggaagtaccaaagaagatgatctcattcagaaatctctgcgtgaccagttggttgagagtatccgaaactcg |
| | | attgcttatcctgacaccgataagtttgctgggctaacacggtttattgatgagtccggccgtaatgtattttttgtggatggtac |
| | | tcgcggtgcgggtaaaactacttttatcaatagcgtggtcaaatctctgaacagtgatcaagatgatgtcaaagtcaacatcaagt |
| | | gtttgccgaccatcgaccccaccaagttgccgcgtcatgagccaattttggtcactgtgactgcccgtctgaataaaatggtgtcc |
| | | gacaaattaaaaggatactgggcgtcgaatgactatagaaaacaaaaagaacaatggcagaatcatcttgcacaacttcagcgtgg |
| | | tttacatctgctgacagacaaggaatataagccggaatatttcagtgacgctttgaaactggatgcccagcttgattactccattg |
| | | gtggtcaggatttgtcagaaatctttgaggagctggttaaacgcgcgtgtgaaattctcgactgcaaagccattttgattactttt |
| | | gatgatattgatactcagtttgacgcgggttgggatgtacttgaatctattcgtaaattctttaacagccggaaattggtggtggt |
| | | agcgacaggtgacttgcgtctatattcccaattgattcgcggtaaacaatacgaaaattacagcaaaactttgctcgaacaggaaa |
| | | aagagagcgtccgcttagcagagcgaggctatatggttgaacaccttgaacagcaatatttattaaaactttttccggtacaaaaa |
| | | cgtattcaattgaaaacaatgttgcaattggtcggcgaaaagggaaaagccggtaaagaggagatcaaggttaaaaccgagccagg |
| | | catgcaggatattgacgccatagatgttcggcaagcaattggcgatgctgttagggaaggccttaatttgagagagggatcagatg |
| | | ctgacatgtatgtaaatgaactgctgaagcagccagtgcggttgttgatgcaggtgcttcaggatttctatacaaaaaaatatcat |
| | | gccacatcggtaaagcttgatggtaaacaaagcagaaatgaaaggcctaatgagttatcagttccgaatttacttagaaatgcctt |
| | | atatggctcgatgctaagcagcatttatcgtgcagggttaaattatgaacagcatcgatttggtatggattcgctctgtaaggaca |
| | | tttttacctatgtaaagcaggatcgtgattttaacactgggttttatttacggcctcagtcagaaagcgaagcattaagaaattgc |
| | | tctatttacttagcgtctcaggtgagtgaaaactgtcagggcagtctgtcaaagttcctacagatgcttttggttggttgtggctc |
| | | tgtcagcatattcaaccaatttgtgaccgagttagcacgagctgaaaatgatagagaaaaattcgaacagcttattagtgagtatg |
| | | tagcttatatgtctgttggcagaattgaaagtgcctcacattgggctaatcgatgttgtgcggtggttgcaaacagccctaatgat |
| | | gagaaaattggtgtttttcttggcatggtgcaattaaatcgtaaatcacgacaacacatgcctgggggttacaaaaaatttaacat |
| | | tgatactgagaatggcctagcaaaagccgcaatggcgtcttccttgagtacggtagcttcaaataatcttatggatttctgtagtg |
| | | tttttaatctgattggtgctattgcagatatctcagcatgccgttgtgaaaggtcagccattactaatgcttttaataaagttata |
| | | gctcagacaacatgtattgttcccccatggagcgaggctgctgttcgtgcagaaatgaaaggctcaagtaaaagtgcagataacga |
| | | tgctgctgttttggatgtagaccttgatcccaaggatgatggcgtgattgatgaaagtcagcaggatgacgcaacggaattttctg |
| | | atgccattactaaagttgagcaatggcttaaaaacgtaaacgaaatcgagattggaattcgtccgtcggcacttttgattggtaaa |
| | | gtatggagtcggttctatttcaaccttaataatgtagctgatcaacataaaaccagactctatagaaatgcagagcatggacgaat |
| | | ggctagtcaatcaaatgccgcgaaaattatgcgttttaatgttttagcatttcttcatgcggtattggttgaagagagtttatatc |
| | | attcggttagtgatagggaatatatcggtgaggggttaagactaaatccagttacttcagttgatgagtttgagaaaaagataaaa |
| | | ataattggtgagaaattaaaagcggataataaaacatggaaaaatacccatccattgtttttcttattaattagctgtccaattct |
| | | acatccgttcatttttcctgttggtgggattaattgttcagtcaaagcactgaacaaagaaacaagtttcaataagctgattgatg |
| | | aaattgttggcgataaattactttctgatgaagaatgggactatctgactaaaaataatgatcaaaaaacaaacactagacaacaa |
| | | atttttcaaaatactataacatcgctgaattcctccacaatcgtcggagcatcatacgataaggatacaccagccaggaaaaccaa |
| | | gtcacctttattaggtgatagcgaagaaaaatgataatggccttcgtataaggattgggtatggaaaggtttcttcttaactcaac |
| | | agttctgttatataggctaagcacagtctctttggatgaggtatcacttgatgagagagtggagtcatctgtattccttgctcaat |
| | | acgaacaggctcgtagtttacctgatcatgtagctaaatctgcttggtcatatttagtgcaacaaatcaaacagcggaatatgaaa |
| | | ctcggcccagtagcaatcttacgcctgatagctgaaaagtttattaaaaacgagaaaggtggccccaaaatcgatctacctatgtt |
| | | ctcggaatggcaaacgctgatgagtcgagtatcgtgtctaccaattatagcgtgtcatcaggtatttaatccagggccagccagtc |
| | | aggaatatagttttcgctggcctttatacccatatcacccgacggttgaagactacattacccgtgaatgcttacatgaaactcac |
| | | caacacctaaatggcagtaccagtgcagaagagtgttggctggatgcactcaaacacccagaagcatgcctcagagattttgagaa |
| | | gggctgggcatctcaagagatgaaacaactctgcgcccagattgatccatctctgacacctagaatcttcaaggatcgtttgcaaa |
| | | tcgcctgtaatattcgcgaaattctttgtcgggttgctcagggcgtggaattgccagagtggatagcatcaatgcaaaatccgcag |
| | | caactggcgaatagcacaattctgcataatggccgggagtatgggtttgcgacagtttggccaattgacgacaaatacagtcagga |
| | | gtctgagttttgctggctaaccggattgttggaaaaatggcggtttaatgcgccagaagggttagaacgattgctttggatttacc |
| | | tgctgattcaaaatcagtacttgaccttactggttcagcgagacgattttttcggatttgaacagttccagaattacaccatgacg |
| | | gagttgagggaggaaacagagaaatcttatttgtctcgttttaaacatgctcatggtgcaggagtgtattctcaggtgcgttatct |
| | | ggaaggacgttttgctccgaagagcgaccccaacaaaatgcaaaagctgctcttcagtgtgttaagaggatattgggaatatctga |
| | | gtgctcatatgtccatggaatgggtgcatgaaaagcctctgactatatcgcaagtgctcgataacctcgaactggttgaacctcat |
| | | ggcaagtgtgtagagctggcgctagtgccgcactttatcaaaagaaagcccaaaaatggtgaggcctatcctcacgcattactatt |
| | | caaagacctgaaaaatcaggcagctattctgatggacatgctgaagtctgaaccgcgtctgacaggctggattcgaggagtagatg |
| | | ccgcagctaatgagatgcacgcaccacctgagttattttgccccttgttccgggtactagccaaatcaggtattgctcattttacc |
| | | tatcatgttggcgaggactttccgcatctgatcagtggtattcgctccattgatgatgccttgagatttttaccattgcgtaatgg |
| | | cgatcgtcttggtcactgcacggcgattggtattacacctagcatctggaaacgctctttgccattgtccttatccatgaccaaag |
| | | agacgagattgctcgatttggtgtttatctggcgggaacttcgaagtcatccggaactgctgcgUacgctagtgatgcagcgattg |
| | | aagctgttcgcttggctcataaagtgttttcgctggaagaggaagtctcgattaccacccttgatcaggtatttgaaatgcggggg |
| | | ctgttggccgaatcggaaggcctactgagtgagctaaatgaaccattaaaacccaaatccctctggttggaagagtatgagcgcgc |
| | | cagagagttggttaaaacaacgggtatgaaaaggccgttgaagttgtataagcaatggctaacatctgacaatgtgcgaaagcagc |
| | | gtgctgaatatgttgaagttgccctagaatatttgccggatgaagcagttgttgcattacaacaagctgtaatggcaaaaatggca |
| | | gaccgaaacattgcgatagaatgcccaccgaccagcaatacacgtatcagtcagtaccgaaacgtcagcgagcatcatatctttcg |
| | | ctggatgggcttgccgggtgaggcgattgaaggtgatgttcctatgtctatttgccttggctctgatgatccggggatcttcgctg |
| | | cggacttgaaatccgagttctatcatctgttcgttgtgttaacccgaaagttcggtttgtcgccagcagatgctttgagaaaggta |
| | | gctgaggtgaacgagaatgggcgcatttatcgctttcatgatgtcagctagcctgtatacattgaggattctgtaattgttcaaga |
| | | ccagcagtgctcattgctaactatctat (SEQ ID NO: 26) |
| 30 | pLG032 | gaggatttatgcacaaaatcctgatgcgaaatgttttcaaaaattgtcaggttaacgttcctgcagatctttgcgttacatgtcat |
| | | ttctggatcctttcccgacaggttaggttgtgattgatatgatgcccatctctcattttagtgatcgttatccctttataaacagg |
| | | agtttatatgttatctatatgcaatagacttaaatcgatatacgtgcgcagcttacgattcacctctctacttactatttaaggaa |
| | | aagagtgaggggagaattgattttcattaagatattatgagagaattatgactagtgaaatagtgttaaatcttgatttcccagaa |
| | | tataaggatgatttttgtactgatagcattgatgagcaagataatgagttgtggcagcaacaggccaataaaaagctactttcgtt |
| | | tctcgaggtgatgggggaggaagcaagacgatataaagaaaataattcccgtagtacgcatccacattataagacattgagtagtt |
| | | atcaccatgcaatctttatcagtggcgcgcggggggcggggaaaactgttttcatgagaaatgccagatttagctggcaaaaacat |
| | | tataataaagatctaaaacgccctaagctatattttattgatgtgattgacccgacgctattgaatattgatgaccgtttttctga |
| | | agtcattatcgcttcaatatatgctacggtagaaaagcggatgaagcaacctgatattgcgcagaatatcaaagataattttatta |
| | | attcgcttaagacgttgtccggtgcattaggtaaatcaaaagattatgatgaatataggggcattgatcgtattcaaaaatatcgt |
| | | tctggaatccaccttgaaaaatatttccatcagttcttgatttcaagcgttgagttactggattgcgatgcgctggttttgccgat |
| | | tgatgatgttgatatgaaaatagataacgcttttggtgttctggacgatattcgctgcctgttgtcatgtccattagttctaccat |
| | | tagttagtggggataatgatctttatcggttcattgccaaaagtaaatttgaggaattattaaatcgtaaagcaaactctaattat |
| | | gctaaagaaggcagcgagatagcagaaagattatcagaagcatatattactaaagtattccccagccatgtgaagatacccctcca |
| | | accgatagatgagttgttgccatatctttatatacattctaatgaagatgaaaataaacaacatacaagctattctgaatttatca |
| | | aacttgtacaacaaaaattctactttctttgtaatgggcaagaacgaagcacaaattggccgcagccgagaagcgcacgtgaagtt |
| | | acgcaactaatccgttctttacctccgtctactcttagtaaggaagatgattcgggaactgatttatggcaacgcttcgctgtctg |
| | | ggcggaagaacgtcgcgatggattagcattaaccaatgttgaatcttatctgtttattaagaatgcgaaagcagtagaagatttaa |
| | | atctgtcaaatcttattgcttttaatcctttactgcaaaaaggaaaatatccctgggcagaaaaggatttttataaacagcagtcc |
| | | caacgtcggaaagagctcaatgcccccgaaacaaattcaggtatccttaataccgtattttccgaacaaaggaaagattttatttt |
| | | aagaagtatgcctgcgctggaactcattatggagcctatgtatgtcactaagacggtagcagaaaaaaatgataattctgcgctta |
| | | tagcgatctatacccattctgattattacagccagcagcagaacagacgatgtcatatattttttggcagagcttttgaaataatg |
| | | ttctggtcagtattagcgaaaactgaaaatcttccacaagaattttatgaaaaagataagtttaaatctttatttggtaatatttt |
| | | caaaaaagtaccattctactcaatattttcaatgaaccctacaaaggttgttgatgaagaaaatgacgatggcagtgaacctgatt |
| | | tttcgcaaaaactggacgatagcattaatgaactggtggaagatatatatatctgggcaaccagtaataaattgcgagccttcaaa |
| | | aataaaaatttaatacccttaatgacgtgcgtttttaataaggtattttcacagatcaatgtactgagaaaaaacgtgcaggacag |
| | | agttaaatttagagatgaacatttgtcagatctggctaagcgatttgagtatatgtttattaatgctatctttactttcatcagag |
| | | aaggggtagttgtcaataccaatgtggcaacaggcgcagctcctgccagagtacgtaatttatcagagtttaataggtatgataaa |
| | | acattatccaggaatatgtccgggattttatccgtgaaagaggataatggcttaacgatagtcaaagagagtgagggcgatatcgc |
| | | agatctgttatttgaaatttggcatagcccattatttaaattaacaaccaggacatgttacccaataggtaaaataaattcgcaaa |
| | | atacggcccaggaaaatttatcatcagattttaattcattttttgaaaatggtatcaacttcgaattgataaaacaatattattgg |
| | | caaacttcaaatcatgataatatcaggacagcagacgttagggaatgggcaacttcacgtcttaatgaagcaatcatccttttttc |
| | | atggatgaaagaaagcaagtctattaaagcgaaaattgacggacagagctacgagggtcggctctttcgcgggcttcagcaggcgc |
| | | tggaaggttatgaggaggtctgagtatgtttaatcaggatccttattggctcattcctaccctttgtctggcatcagaccgaattt |
| | | tttatgcacaattgcgagaccacttaggccagaaaagtagcggtgaacgcaaaaaagaaaaaaatggatatatactggtacaggcg |
| | | gcacaagactatcaattctattttggcggccgtattcggaaagaggatgtgcaaaataatgccttaatgtggcagatagaaactgg |
| | | taatgaaaattgcttatcgatgcttgatagtttgtcagcatatttcctcacatggcgcggcaattgttttgaggtcaggcgtgagc |
| | | gacttgaaccctggctgatgatctgttccgtgatagatcccgcatggattattgcctatgcataccaacaattgattaaacaaaat |
| | | gttgtatgtgatagtgagcttatttctttgctgacagaacatcaatgtccatttgcctttccaaaaggcagaggggacatttcctt |
| | | tgctgataatcatgtccatcttaatggtcatggttatagttcaatttcaatgctgaactttatagatggaaattataaggttaaaa |
| | | aagggataaaatggccctatcggcaggaatacaccctctttgaaagtggtcttctggataaaaatgatcttccccgctggctgtcc |
| | | gcttatagctcttgcttacttaaaaatgtatataattcatttcaacaaggaaaaagatccgaggtagatttcacatgtctgaagga |
| | | tgcggtcgaaacggtgcttgcggatgaggataaatattattttttagaggtagcttcgctatatgatgttgtcaccttgcagcaaa |
| | | gagtgctttatgaagccgcccagcagaaatatcactcacatcaacgttggttactgtatacttgcggaataatgttaggtacagaa |
| | | tctgaagattatgcgaatgcgctggctaacctgatccgaatcagcaatattctaagaaactatatggttgtatctgcggttggatt |
| | | gggacaatttattgattttttcggcttcaactatcgtcgaataacaaagccagctgatacaaacaaccgagttcattatgattctt |
| | | ctgctggtatttccagagaatatcgtgtctctcctgattttgtactgggtagcggcgtaatgcctgatatatatgccaggcaactt |
| | | ttcgatttttattgtacccaagcacgcaagggcgtacccgaacaaggacatattgttgttcattttacacgttcctttcctgacaa |
| | | aaaatcaacatatgataaattgctaaccgagtgtcgcgaacggttacgttctcagtgtgattattttggccgttttttaacatcgc |
| | | ttactttgcagtcgatagaatataaaaatttatctactgatgaagatcgaagcatagacattagaaaattagttcgtggctatgat |
| | | gttgctggaaatgaaaacgagctacaaatagaggtatttgccccggttctccgggtactgcgtgctgctaaatttaaaggggaggg |
| | | ggtgaactttaaaaggctacagcgcccttttattactgtacatgctggtgaggattattgtcatatactcagtggccttcgggcta |
| | | tggatgaagccgttgaattttgtatgttaggagaaggcgatcgtatagggcatggattagctctgggagtagatataaaactatgg |
| | | gcgaatcgccaaaagcgagcatacctgacggttggacaacatcttgataatttggtttgggcatatcatcaggcagtattactttc |
| | | tcaacatattgtcgagcatataccagtaatgcatgaattaagggataagatccattattggtctcatcaattatatagtgaaactt |
| | | atacgccagatttactctttaaagcatggctgctccgccgtaactggccggattataagtcaatcatatctgatccagcaaatatc |
| | | aatgaatgggtgcctgaccaacatattttagtcagtacagatgagactacagctaaggccagaaaaatttgggaacgttatttaaa |
| | | tagcggtctggcagaaaatgatgtttttaacagaataatttcagtaaattgtgcgcccgatacagcgcaaaatttttcaatgacct |
| | | ttaatgaaaatgaagatattttatccaaaggggaattattattgtatgaagctatccaggatttcttaatcgaaaaatatagtagg |
| | | ttgggtttagtcatagaagcttgtccaacctcaaatatttatattggcagactggagaaatatcatgagcacccattattccgttg |
| | | gaatcctcctgactcccaatggattaaacctggtgggaaatttaatcgctttggattgcgcacaggacctttatctgtctgtataa |
| | | atacagatgacagtgcattgatgccaaccacaattgaaaacgaacatcgcttaatgagagactgcgccatacatttttatggtatt |
| | | ggaacatggatggcggatttatggataaactcaatacgcataaaaggtattgaaatattcaaaggtaatcatttaagtcaggattt |
| | | agataatttaatctaaatgtaaacaagaaatccacgcaaatgcgtggattttaagtcaacttattattctctgaaacggtttaacc |
| | | gttcggaacaacagattaaatc (SEQ ID NO: 27) |
| 31 | pLG033 | tgtggttagttatcacagcactaacctattttcgagctttttgattgaccaataccatttcttttaattatgaataatgatgcgtc |
| | | aaccgatggcgaacgggccaaatccactcttctacaactgcccattgtcacggtgtggaataattaaaaattttagatttttgaga |
| | | ttattctcattaccatcttgattttatttggttttgcatcaaaattcatagttcacaagcttttctcactccaaaaacaactgtaa |
| | | agggattattgtgaacacgatatacataccattagacagcggagagtctgcggttcttaaggatccagataccttacttccccgaa |
| | | atatttacgaacagcttactcgatttattgaaaaggctgttaatgaagtaccgaagcctcacgaagcgcttaatgaaacccgtagc |
| | | cataaggctatatcgattgacggcgcaagggggacaggaaaaacgtcggtgctagtgaatttgaacgactatctgcagagtaatgc |
| | | tcagcaactggcggggaaaattcatatccttgatcctatcgatccgactctacttgaagatggtgagtcgctgttcttgcatatta |
| | | ttgttgctgccgtgcttcatgataaagagatcaaaactgcccaaagcagagacctcgataagtccagagtgtttacccagaagctt |
| | | gagaacttggcacacggactggagtccgttgatttgcaacagaatcaacgtggaatggataaaattcgctccttatatggcagcaa |
| | | gcatctggcaaattgcgttgaagagtttttaaaatctgcgttggagttgatcggaaagaaattattgatactaccgattgatgatg |
| | | tggacacttcactaaaccgggcatttgaaaatctggaaatattgcgtcgttatcttacctctccgtatgttttgccggtagtgagc |
| | | ggcgatcgccgtttatatgatgaggtctgctggcgagattttcatggaaggttgaataaggattcagcatataatcgcaagaacac |
| | | atatgatattgctagagatttggcaattgagtatcagcgtaaaattctgccgctaccgcgcagactgagtatgcccgatgtaagtg |
| | | attactggcagcaagatggtatcgaagttacgctagataaaaatggcattcctctgcgtaattttatggcatggttgaaaatattt |
| | | attactggccccgtgaatggccttgagggtagtgatttacctctaccgataccttcaatacgtgctttaacccagttcatcaacca |
| | | ttgcagggatttaattcgtgagcttcctgaaccattcagaaagaaagtcagtacgctggccttacgtcgtatgtggcaaatgcctg |
| | | atgttcctcttgatgttcttgaaagttttgctgaaaaacatcgggaattgagtaaagaagctaagcgtgaatatggggaggcttac |
| | | aagctattttatgatggactaaagaattttactgcttgggatagtaaggcttatctagaagatgataaacaatctgcatggctcga |
| | | taggttgtgtgagtattttcgttttgaacctaaggctggggctgtgtttttaacgcttcaggcaaaacagttctgggtctcatggg |
| | | cgcagggtgacaatcgtaatcaatcgattcttgcgactccgctttttcaacccttattgcataattttcgtgaatacgatgtcttt |
| | | gaaaggtatgatgatctttctgattgggaatctcagttaagaacaaggttaccggagagttggttgactgccattaaagggcaaaa |
| | | aacgcttttaccctatcctgtagcagaagcgggaattaataccagtttaaagtggaggtattgggaagaattagagaactatgggt |
| | | ttgatcctgctttggaaagcaaggcaaatttccttttgtccacgttgatgcagaggaatttttatacaaactctaaacagtcagtc |
| | | gtgataaatattggtagagtttttgaaataattattgctagtcttgtttcggatttagagttggccgacttgcagagaattagaca |
| | | acgttctccattttactctgctagcgcgcttgcacctaccaaaacgttagatttggaagaggattttacgaaaaagaatacaagat |
| | | ttatgaataacagaagtgaaactgacagagacatttctgatgatattcttgttgatgtgccggataaaaatgaggacgcatggaaa |
| | | aaaatttgtgatgaaataaaccattggagaaagacacacaatgtggctagtacaaacttatcaccttggctggtttataaggtctt |
| | | taataaaacatatagtcaggttgctaataatgtgtttgttcccagtggaatgcaaaatgttgatgcggctctaaatgtttttggta |
| | | gggttttttatgcagtttggtcagcatttggtagttttgaaaaaggcgaattgttcggactatccgatgtggttgctacaactaat |
| | | attatttcggcaaaaaatttttataatcatgataacttccgagtgaatgttggaccgtttacgcctgagcaaaaccaaaattctga |
| | | cagcgatcgtgaggcatatcagcatcgcaaaatgtatggtgaaaaaaccagagcggtaagttatgtattagcaactcatccgctga |
| | | aaaaatggatcgacgaggtattacgcactgagtttaaacaaaaacagaatgctcagattcagaccgagagaaaaatgccgattcag |
| | | gctgagaaaattatagatatcagcccggcaagagagtttatcacaagaaaactttcattaaattcacactcccggttggttaaaac |
| | | acgtataataaaacagcttaagatgttatatccaaactacgataaggctaaggacttcattgatgaagttacaaaccacttccctc |
| | | agaatgatcccgcaattaatacgcttcagaaagcatttgcagaactttaccccgatggtgacaaataatgttaactcggtctctaa |
| | | gtgaacatgctgcagggtgttttttcactgatgagcgtctgtcacaacgctttctagatatccttttatcgccacccaaggatttt |
| | | gaaacgtggtcatcattgcaggaggaatctttcaagctgctcgttaagagcatcgatagccgatatccacgcacttaccggttaac |
| | | cgacgtacgccagcttgtggggaacatatgtgacaacgggttactgacgagtccgacactaccttggctcgatgtcattgcggatc |
| | | agttactgttgcggaatggcgacttactctattaccgcgaaaataaggttcaagactacgtgcgaatagctgcggaactcgaccct |
| | | gcccttctagtgggatggcgtcttggcgactggcttttgcaaagcccaccgccgcgattgacggacataacccgtgtggtgatggc |
| | | gcagaatccgttttttgctccacctgctaatgcaggtaaaccttttgccgaggggcacgtacatctcgggggagtgacggctggag |
| | | atactattttggatggctatctttttgaagagattgaactacccaaaagcaaagatatgttgttgtgggcgcacaaagagcatgat |
| | | gagttaacaccgttgataaatcgagcaaagtctttgcttacagttctactttctgccccccctcaaacggtttctgagcaaactca |
| | | aaatggttttgatcagcgtaaaactgtatctgagaagtacaaggcattacagaacccaatggatagcatccatcgtctcccagact |
| | | ggttattgcttgctaaaaagaatcgcggaactgaaagcgtcagccccggctggtttttaaaccaactggcgcatgcctccgaaaaa |
| | | aaacatccctcgcgctggctgtggctgcagctatacctttgccactcttatcagcttaaagacactcatccactggagcgcacggc |
| | | aatactctgtttttggcttacggtaaatgcgctacggcgtcacattattatggacggacaggggcttgcgtgttttaccgagcgtt |
| | | attttaatggtgctttacgtgcgggtaagaaagctgacagtagcaatatgcgctacctgtttgccggtaaagacgatgtggccgaa |
| | | gtgaaagcatccccaaaggctttcgatcatgagatggtcactggattttcctcgacattgctgaaaaccctcggcattccagctgt |
| | | ttttccaccgtatatttttggtgagcatgagattaagccagatgaacgcgtgctgcgctatattggagcactggagcgctggcagt |
| | | tttgtgggcacttttctcgctctaaaactgcaagtcgcggcaagcgagcaaaggctgatttgcaggctaactggacagaagcggag |
| | | cgattgttacagaaactgtacagtcataatggctggaatcatcccgtcttcttagggggtaaacgtaacccacattttcattttca |
| | | gccgtcgaactggtttcgggggcttgatgttgcaggggatgaaaacgtactaaaaattgcaggctttgccccgatgctgcgctggc |
| | | tacgaagtggattatatcccgtaccagaagggcttcgcgccagtatgagttttcatttcagtattcatgccggggaggattacgca |
| | | catccggcgtcaggattgcgtcatattgatgaaacggttcgcttctgcgaaatgcgggagggagaccggctaggacatgctctggc |
| | | tctcggaattgaacctgcgctctgggcgaaacggcatggtgaaatgatactacctctggatgaacatttagataatcttgtctggc |
| | | agtggcactatgctacgcttttatcggcttcattgcctctcgctcaggcggtattaccgctgcttgagcgtagaattgcacgcttt |
| | | attgcacggtgcgaatggtgcaaaaagagacctccgcaaatagataacagtgtggtggggaaacaggcctgtagtgatgataaacc |
| | | tctggaaaatattacacctgatacgctctaccgggcctggctactgcggcgtaattgttcatatcgactccagcaactccacggcg |
| | | gttcccctttgacctcgcaagagaaatgtgcgctgccggattgggccacgctcagcgataaaggcaatgtggcggcgcagctttat |
| | | cagcaaagacactcgagtctccttgacgatatgccgccgcaactggtagttgtgcgtgtagcggacgaatggggaactcaggagct |
| | | tattggcttgggaaatcctggtaaactgcgtcagcaggctcttgacggtaaagatatcctccaagacattgatacgccggtagagc |
| | | tgcaatttatgcatgctttacaggactatttgctagatcactatgatcgtaaagggttaattatagaaaccaacccaacatcaaac |
| | | gtatatatcgcgcgattcaaaaagcacgtagagcatcctatttttcgttggaatcctccggatgaagaactgttgaaaccaggcgc |
| | | tgaatttaatcgttatggattgcgccgtgggccagtcagggttctggtcaatactgacgatccagggattatgcctacgacattac |
| | | ggacggaatttttactactgcgagaggctgcgattgagcgtggtgtcagccgaacgatggcagaatattggctggaaaggctgcgc |
| | | ctgtacgggctggaacagtttcagcgtaatcatttaaatgtatttgaagttattgaatagaggattttatcgtgagtggtacattc |
| | | ccttacttgcaatatacggatgtcaatgggctacaacctaagctcaaagaagagttgaaaaatttacggagaaaagagtatttgtc |
| | | ctactggcctcgttttctgatacgtagaatttcgctttatgctcttccattcctcatgttcttcacttttttcttttgtctgagtc |
| | | tgacgaagaaagttggggcagaggaagtgactaatattcttggaaccgtgagtatatccttcagtagttgcctgctgctggggatt |
| | | attatttctggtgtcgtgttactcttgcagtggacgtgcttcaactgtaaatacagtccgcaggatacgaatggagttgttggggc |
| | | tcgtaagttaaattataaattacttgctcatgttgtatttgttattgcatgcgtgcttttatttgtttttatttattgcaccaata |
| | | ataaagtgttttatggttttatcgtgtttcttggtttgacattattaccattggtaattgaccgtaccttgggggtgactcgtcaa |
| | | aatgaacgtcacaaactctatatcagaaggttagagcgcctcgatgaattgaatattctccgggagaaaatgaatattaaattcga |
| | | agaatcccatttcatcgagtatatgaagcttgttgatgaagctgatcacggaaaaaaccaggatacagtaagcgatacatcctatt |
| | | ttatgacgttgatagaaaataagctaaaagtgtaatcggttttaatatgatgctgtataaaaaactacgcaattgcgtggtttttt |
| | | gtcggactatgagggcaaggttgccctaaaacagaggttaaacgttgggatgtgatttattgcacatcatgccgtgcccatccagt |
| | | agaatccggttcgaaatgtgtataggattgtgtatatgtttctgttcggtctcggattcttatacac (SEQ ID NO: 28) |
| 32 | pLG034 | accgtgctggcatgtttttacggagtgacgctttcattaacctgtacacgaacttctattccggcatcatgacaggcctgcagcca |
| | | ctgcgccacttccagcggatcgccctcccggcgtaccactctgccttctttattccataactgcagacaggtgctgccgtcgagacg |
| | | caccacaaaatccccacggcaggcctgataggggtttgagggccaaccgtacgaaaacgtacggtaagaggaaaattatcgtcttaa |
| | | aaatcgatttatgctatcacagtcgtctcttcaggtaagtacggttgcctttgcctgctttcttctcgtctggttaagttaagaaat |
| | | tcagagatccatgcttgagataaaagcggaataaaaccagtaaaatgtaactaaaacaacaacggaattgtatcaatgataatgtcc |
| | | acaccgtggctgacaccgatcgttgccgatagtgatcatgctgaggcaaatgcagtgagctatgaagcactgactccgacagaactc |
| | | gactcagataaagcaggctgttatatcagcgcgcttaattatgcttatgaacatccggatatccggaatattgctgttaccgggccg |
| | | tatggggcagggaaaagctcagtattaaaaacatggtgcaaagctcacaatgggacactgcgggtgttaaccgtttctcttgctgat |
| | | tttgatatgcagagacatgtggatgaaagtaatggggacagcagtagtgacgaagggacgaaaaatactggtagtgttgaaaaatct |
| | | attgaatacagtattctgcaacaaatactctacaaaaataaaaagcatgagcttccctgttcccgcattgaccgtatatcagatgtg |
| | | actgcgggacaaatattgcggtctgcgtcttttctgacaggaaccattttactgagtggagctgctttatttttccttgcgccggat |
| | | tacgttacaacaaagctatctttgccgggagcattcgcccgttaccttcttgaatgcccgtttggggtgcgtgtgtccggtgcagtg |
| | | gcatctgtgatgggatcgttatgcctgcttttgaaccagttacatcgtatcggtatatttgacaggaaagtaagtcttgataaagtg |
| | | gaccttctgaaaggcgctgttacaacccgggcatcatcaccttctttacttaatgtctatattgatgaaattgtctatttttttgat |
| | | tcgactaaatatgatgtagtgatattcgaagatcttgaccgttttaacaatggccggattttcgtgaaattgcgggaaatcaatcaa |
| | | attattaataactgcctttctgacagaaaacctgtaaaatttatttatgctgtcagagatggtattttcaactcagcagagtcaaga |
| | | acgaaattctttgattttgttatgcctgttattccagtgatggataaccagaatgcttatgagcattttgttaaaaaattcaaagaa |
| | | gaagagataaataataacttaagcgaatgtatttctcgtattgcgacatttattcccaatatgcgtgtaatgcataatattacaaat |
| | | gagtttcgactctatcagaatttagtcaatagtcgggaaaatctggccaaactacttgccatgatagcatataaaaatctctgtgcg |
| | | gaagattatcatggtatagatagtaaaaaaggtgttctttatcattttattcaaagctacttagaccatgaaattcagaatgaatta |
| | | ttacattctgcaaataacgaacttgaggatatggcacagtcacttgtagcgataacaaatgaaaaactcgcaaaccgggaaaatctg |
| | | cgcgaagaactgctcatgccttaccttagtaaaaattatagcggcgcgcttgttttttatacagaaggaaggcaaataagtcttgat |
| | | gatttgatacaagatgaagatgaatttctcatgcttttagataaggaaaatattcaggtcgttaccccctataacagacaaaatttt |
| | | ctcatgataaatcagcgggatacagaaaaactgaagcagcagtatgaaaaacgatgccatttaattgaaactaaatctgttgataat |
| | | ataaccagagtgaaaaataatatttccagtctggagtcattgaggaccgaaattctttccggaactgtagctgatatagcagaaaag |
| | | atgacaaatgaaggctttgttgcctggataaagaagaaagaggatacaggtgtcctgacgattcagtcggaacatgaacagattgat |
| | | tttatattttttctgttatcaagtggttatttatcaacagattacatgtcctatcgctcaatcttcattcccggagggctgagtgag |
| | | acagataatttatttcttaaggatgttatgtctggtaaaggtccggaaaaaacattctcattccatcttgataacgttaataatatt |
| | | gttgaacgactcaaaaagctgggggttctgcagcgtgacaatgctcaacatcctgctgttatcagatggctgattgataatgaccct |
| | | gataccctgaaaaacaatataatggcattactgagtcagacgggtagccagcgtgtggttagtttgctgatgttgatgcagaacgat |
| | | ttcacaacgtatgttcgcctgcgttacctggagatttttatgtcagatgaacatatactgaacagattgctggcacatttatgtgcg |
| | | tcagaagaacgcacacccgagcaaaagttttttgttcaggaaatagcggcacacctgttatgcctgactgaaaaatcaaatatctgg |
| | | caatcggttgagattaataaacgtatcggtgagcttatagattcctccccaattcttattactgctgtgccaaaaggatatggtgat |
| | | gcgttttttgaagtgttgaaagataatacactttcagtttcatatattccaggtgatgtgggagacgagaagtgttctgttatcagg |
| | | aaaattgcgggtgcaggattattcaaatattccgtcagtaatcttaaaaatgtttatctttgcctgacgcaagacaagaatgaagaa |
| | | agaatgtcattctctctttatccgtttcattgtctcgagtccctggctatttctgaattaacagaaattctgtggactaacatagaa |
| | | gattttattttatcggtatttattgaatcggaagagattgatcgtattcctgaattgctgaattcttctgaagtctcaatgactgtt |
| | | gttgaacagattatagccaaaatggatttttgtataaataatctggatgatattattaatcgttcagagtgtgcggacaataatgct |
| | | tcagggagaaatatctatagcatgctgttgcagcatgacaggatttttccatcctttgataatattattcatttattgcatgataca |
| | | tcaattaatacttccggtgaacttgttcagtgggtaaatgagaaacactttgaatttgaaccatctgatatagtcataaatgataca |
| | | ggaatatttaataattttatttctgaattaatttgctcgccagtcatttcagaagaagctttactgaaagtactgagtaatttaaac |
| | | gttgttattatcgatgtgcctgaaaacattccattgcgaaatgctgaactgttatgttcagagaaaaaactggcaccgacagttaat |
| | | gtctttacggtgttgtttaatgctctcagtgaaaatgttgatgatattaacaggatgaatactctgcttggtaaccttattgcccag |
| | | cgtcctgagattattacccaggagccagaagatattttttatatcgagggtgactttgatgaagaactggcaagcgaactttttcgt |
| | | cacaagctaatcggtatgaatataaaagttgccgctttacgctggttgcgtgataacaaaccgggaattcttgataagagctacctg |
| | | ctgtcattagatattctggcagaactgagtccctggatgggtgacgatgatctgcgcctgacactgcttaaacgttgtctggttgcc |
| | | ggggatgctggcaaagacgcgctttgcgtggtgctgaacagttttgctgatgagagctatcatggactgttaccacatgacaggttc |
| | | aggaaaatccctcactccgtggatttgtgggaagtggccgaattaatcagcaatcttggatttattcagccgccaaaaatggggtca |
| | | gggcgtgatgaacacaaaattgttattactcccgtacgctatgtccgtgatgttgagttttatgactgagcatcattgatacggtgt |
| | | tttaattgccttaaatacaaaaataaaaacagattaatgcttaatgtgcattaatctgttttagttatcaatggctgttaattattg |
| | | ttaattttacattaatctttctttttcttcaggaagatccgaaaactcctggtcacggatcttcct (SEQ ID NO: 29) |
| 33 | pLG035 | attatctgccaaccgataagatggctgcctaagtcgtagcgattcagcactgttttagcggcgctcgattgcaaagtcgtgctttg |
| | | ctgacttgcgattgtgctctttacgagcaaagctttcaggtatagtaagtgctaactgtagtgtaaaattatagggatagatgaag |
| | | aaaacaacgaggctttagctaatctttgcagttgtgtctgctataataaggcgaaattttatctgcatgattttgtttgattaact |
| | | ccgaaagccagctctctcggtgaagattgggaagggatatcaatgagtgatgatagctataaatttcaaaagttaacgccgttcag |
| | | cgatgttgagctgggtgtatataaaaatgcgatagattttgtttttgccaataacgatctaaaaaatgttgcgatatcagggcaat |
| | | atagcgcaggaaaaagtagtcttatcgaatcctataagaaaagtcattcaaatataaagtttgttcatatctcacttgctcatttc |
| | | agatcgattgaggaagctgaaactaatgaaccaagtaaagatataaatgaaaccgcgttagaaggtaaagttcttaaccagttaat |
| | | tcaccaaattaatgctgatgatattccccagacacattttaaagtaaagaaaaaaataaaaactaacaacattgtgataaacacca |
| | | tctttacggtgttatttatcgccatgatactacatatcacgctatttaataagtgggaaaagtttgtttcacttttatctgaaggt |
| | | aatataaagacactacttacattatcaactaaatacgatacgcttttaattagtgggtttatatgtactatcctatcttgtatttt |
| | | catttacaagttaataaaaacccaaaagaatcgtaatgttcttaagaaaataaatttacagggtaatgaaatagagatttttgaag |
| | | aaagtaacgagtcttatttcgatagatatttaaatgaagtattgtaccttttcgagaacgttgatgctgatgccattgtttttgaa |
| | | gacatggaccgttttaatagtaataacatctttgaacgtcttcatgaggttaacagactggttaatattcaacgggacacagcagg |
| | | gcacaagaaatcgacgttacgttttatttacttgcttcgtgatgatatcttcatttcgaaggatagaaccaaattctttgattata |
| | | tcattccagttattcctgttgttgatagttctaactcttacgatcagtttatcacacattttgatggtggtggtattctcaagttg |
| | | ttcaatgaaagatttctacaagggatgtctttatatattgatgatatgagaatattgaagaatatttataacgaatttcaaattta |
| | | ttataacaaattaaacacgacagaacttgactgtaataaaatgttggccattattgcctataagaatattttcccaagagatttta |
| | | gtgagttgcaacttaatcaaggtatggtttataccatatttagtgaaaaagacaaccttattattgaagaaataaagaaaatagaa |
| | | aaagatattagagatagaaaaaaagagattgaggcaatcaatgatgaaatactcaactctagtcaggaggttgatgctatatacga |
| | | taaggaattatctagatataataatcatcctcactataatcaggctgagaaagctgatatagcaaagagaagggcggctagaaaag |
| | | aaagtgttgaaaataaatttaatggtaaaatagaagaaattaatgagcttatatcaagatcaagagaaagtttggttgattctaga |
| | | aacaaaagacttaaagaagtaataactagagaaaacattgatgaaatatttaaactcacctataccaatgaaattggagaggaaag |
| | | agactttaatgaaataaaaagcagtgagcattttgacttgcttaaataccttattcgtgatggttatattgatgaaacctataccg |
| | | actatatgacctatttttatgaaaatagcctgagtcgaattgataagatgtttttacgcagcattaccgatcaaaaaggcaaagag |
| | | ttcacttatcaactcaagaaccccaagctggtcgttgcccgccttcgagaagtggattttgaacaggaagaggcgcttaattttga |
| | | tttattagcttatctgcttcaaacgccagcccaggtaaacttaataaaacgtttattcaaacaactaagaaaagatagaagagttg |
| | | agtttattcgtggttactttgaaactgagagggctcagcctgtcttcattaatcgattaaatacacagtggcctgagtttttttct |
| | | tatgcgctgacagagagtgaattttctgctgattgggttaaactctactctataggcacgttttattattctgccaatgacgccat |
| | | cgaggccattaatattgatgattgtctgactgattacatctctgattcggcaggttatttagcaatatcagaaccgaaggttgaca |
| | | aattaattagtggttttaagttgcttaacgtctcttttgtcagtattaaatUgaaaacgcaaataaagtactctUgatgcggttta |
| | | ccagcattcactttatgatattaatttttccaacctgaccttaatgctgagtaaggtttacacgcttaatagtgaagatgatattc |
| | | gccataagaactatacactagtgatgtcacaacctgattctcccttggctagttatgttaataaccatattagggactatctggat |
| | | atggttttatctagttgtgatggttcaatcgtggatgatgaatccattgttttatccgttcttaataatgagggaatatctgatga |
| | | acaaaaaggccagtatataaacgctttgcaaactttcgtgacatctctgagtgaggttgagagcgaatctttatggtcatctttgt |
| | | tggataaagatagagcagtgtgctctgaggaaaatattgtctcttattttgaacatgttgatggactggatgactcacttatcgaa |
| | | tttatcaatagaactgatgtagacctgaattttcaaaatattaatattgataacgagcttaaaggtaaattatttaaatcgattgt |
| | | tatctgtaatgatttatcaaatgataaatatgaaaaattaatttgctcactaaatattatttgtaaaacatcctttagcgctagta |
| | | atatcgcgagtgataagttcaaaatattagtggataaaaatattattcgtatgaatgttgcgccacttaatttcatacgagataac |
| | | tattcagagcaactttcctattatattcataagaatatcagggcatacgttgaattaatgacgattgataactttattttggatga |
| | | ggctatatcaatactttcttggaaagttgatgatgatttgaaagttaagctactcgagtttgttaaaactccgttggctatttata |
| | | gtaagaattactctcaggtcgttaatgactatattttagaaaataattttaaaccagatgaacttctaatcttgacgtcatcttat |
| | | aaaacttggggaacctctactcagtcgctcatcttgagtcgagcaatacaggatatatcagcattgatagcaagtcctaatgatgt |
| | | ttctgaaccgttactaaaaaacctgtttgtcgcagagggactgaatatgcagaataaaatagcactgctaatcgctttgttgccgg |
| | | gtaaggatttgagtaagacgacttgcaaagagtatcttgatctgcttggtttatcggagttcagtaaaattttggggcgaggcaaa |
| | | cctaaaattgaagttgattcaactaatcaaagtttattaacagcattaagagataaccacttcttctctgattttgaggtggataa |
| | | tgaaaatcccacttattataaaataacaaggcggcgctctatgtttggctcagatacatagcattatgtatttttctacagtttgg |
| | | gcacttttatagtgcccaatttttacgctgaaacttacgcagataatctgactttttcccagttgacgagtacacctag |
| | | (SEQ ID NO: 30) |
| 34 | pLG036 | atctatagcagtcatcatattggattattggtgaagtggtacactgaatttgcccacctgaacagagttggttttatcaaacctgt |
| | | agtttactcaatgacgtaaaaattggtgatgtaaaggatataaaaatgtggtcagacaaagagtcatcagaagactacctaaattt |
| | | tggtgaagtatctcagttagccgtggatgtacttaccacgaaagatatgttaccagtatctatcggaatttttggaaactgggggg |
| | | caggtaaatcctctctgttaaaactgatagagcaaaaacttgagcaagacgacaaagattggattgttatcaattttgactcttgg |
| | | ctctatcaggggtacgacgacgcccgtgccgcacttcttgaagtcatcgctacagaattgacaaaagctgctgaaggtaattctac |
| | | ccttatatcaaaaactaagagactccttagtcgagttgatggttttagagctatgggattactagctgagggtacagctttaatgg |
| | | caggattacctactggcggtttgctttctagggggattggtgcattaagaaatatcaccgatggcatccagagccaggaagagtat |
| | | gaggctttaggcaatatagctaaagaaggtaaagaaactgcttgtggtttgattaaaccacaaacaaaaaaaagcccccctcagca |
| | | gattgatgcctttcgtaaggaatatggggaaattctagaagaacttggaaagccactcattgtggtaatagataacctagaccgct |
| | | gtctccctgccaatgctatccatacacttgaagctatcaggctattccttttcttgactaatacagcctttattattgcagcagat |
| | | gaggacatgattcgctcttctgtggctgattacttcaaaggggcatcacagcgccatcaaatagattatctggataagctaatcca |
| | | ggttcctattcgggtgcctaaggctggggtccgtgagatccgttcgtatctgttcatgctttatgccattgaacatggcttagaag |
| | | gcgaaaaaataactatgctccgtgagggcttagagaaggcgttacagcaatcctggaaagatgaaccaatctcacgtcaggaggcc |
| | | ttaaaaatgactggtgaagcggatgatagcaacctcgcgctggcgtttgcgcgtgctgaccgtattgctcccattttagccaactc |
| | | tccaattattcatggtaatcccaggatcgttaaacgcttgttgaatgttgtgaaaatgcgatctcaaattgcgaagcgacgagcaa |
| | | tgcctttggatgaagcaattattactaagctagtaatttttgaacgctgtgttggagtggatggcaccgctgatttatatcatctc |
| | | gtggatattgaacaaggtgttccccagatacttaaacagcttgacgataatggcggtcaaatacctactgatgcaccaaagacatg |
| | | gactgatagtccaacgactaaatctttcatcagtcaatgggcccaacttgaacctcgtcttggtgggattgacttaagggccgcca |
| | | tatatctgtcccgagaaactatgccaataggtgcatatgtggttggtttatcgccatctggacgggaagtactaaatgcactaatt |
| | | gaattgaaaaacactagttctcctacagcagaaaaccttttgaaagcacttcctcgtgaggagcaaatacctgtaatggaaggttt |
| | | aattaaccagttacggcaggtatcagattgggatcgtaagcccagaggcttttccggcgcatgtctgttggcccgctactcaacag |
| | | atgcagccagcatattaattcgttatctacaggaattacagttggggatgaaacgaccagcgtggatgactgcagcattaaaagat |
| | | gaacaatggaataaggacgcttaatgggaacatcacaatcaagtaaaggtccaggaggtggctctccgctggttccaccatgggct |
| | | gatgatcagccacagcaaccgttaccctcgccgcaagaaaggaggtttgcgccatttcgagaatcgttgggaaatgcggtatcaaa |
| | | tggaaatcgagcagatttcagaaaagccatagggcactacgcgcgaaaagcctccggagggagcagtaacgctgctcggcgattag |
| | | ggagtgtcacgcaagctggggccgaattatttggggctttagtgggaatgccttcggctcccggagaaccaagcatcgatttgggc |
| | | agtttggcaggccttccatgcgaaatagcaatatcaactattgctcaagctttaacatcacaggatggtgactcagaaaagatctg |
| | | tgcggccatgaaccatgctttagtggaggctcttgatggcgtagaaattttcgatcctcaaaaaataactgatggtttgattgttg |
| | | acacaatgattggttatctagcggaaagtattttccttcagatggtaatggattctaatagggcatggaacaaagcagatacacct |
| | | tcaaaggcaattcatgcagaaattgaactccgggaattgattaaagttgttgttgataaacatatggcaccaaaacttgccggtaa |
| | | cataagatcgttcacacgaaaccaaatggtaaaaattgaacgtcaggccattattgaggcctggcaagaatgggaggcataccagt |
| | | gacacaattagttttccatcataaacatcaccatttgccgccagcaagtgagaaagtgttacctgttcagctatatggattaagtg |
| | | gtcagaggcgcggagatatatctgttatcgggaatcctgcgattgatcggatcagacgtttgggagtacagcttccagctaaggtc |
| | | atggattttctgagtgttgcattagcagtaactgcagcagatactttcgttcagcgtgaaagttccgaggatggttggacccgcca |
| | | attgtcgttacgactcccccttcatgaaccatccagatggattagtctaaagaaagaacttgagagtgctttgcattttcttagtg |
| | | gagacatctgggatttcgaattttgtgacgatggttatgcaccgccagagccttatagccagcattcaaggcatcgtctgattaag |
| | | ctaaaagggcttgactgtgtcagcttattttcaggaggtctggattcagctattggtgcaatagatcttctggctgcagggcgcgc |
| | | tccacttttggttagtcatgcttataaaggggataagtctcgtcaagatcagattgctgaaaaattaagtggccaattttcgcgct |
| | | ttgagattaatgctgacccacacatttatcaaggcgtgactgatattacgatgcgaactcgtagcctcaattttcttgcccttgcg |
| | | gccgtaggtgcttgtgccgtacaagagatatctcaacaagaaaagattgatttgttcgtacctgaaaatggatttatctcattaaa |
| | | tgcaccacttactccacggcggataggttcgctgagcacacgaacaacacatccacattttattacgagcatacaaaagatctttg |
| | | atgcgctcggtatttcttgtcaaataatcaatccatatcagtttaagacaaaaggaaaaatgatctccgaatgttcaaataagcag |
| | | ctcttatctaaaattgtggaaagtacagtatcctgcagtcattggaaacgaatggggcagcaatgtggggtatgtataccgtgtat |
| | | cattcgacgagcatcacttcatgcagggggaattagtagagatgttgaatatattttccagtccttagctaaagtaatgaatgaaa |
| | | tagatcgcagggacgacctgatcgcccttaggattgcgatcacgcagaaatcgactttgaaaataggtacatggattgccaaaagt |
| | | ggccctttgcctacggcagaatttgataatttcaagcaagtatttaaggatggcctagatgaggttgaaagctatttactgagtga |
| | | gaacatagtatgagcatcgatatgcactgtcatctagacttatatcctcggccagacctcgtggctgaagaaagtaaacgtcgagg |
| | | gacttatattctgtcggtgacaacaacacctaaagcatggcatggtacttctttattggctaaagaaagtcaacgaatccgaactg |
| | | ctcttgggctacatcctcaaatcgcgcatcaaagatcgcatgagttagacctgtttgattcattgctttcggaaactaagtatgta |
| | | ggggaaatagggcttgatggtggacagggatttaaagaacattgggatattcaattgaaagtgttccgacacattctcaacagtgt |
| | | aaatcgggctggtggcaagattatgactatccatagtcggggaagtgcatcagcggtgcttgatgagattgaaaatatcgatgggg |
| | | tggcaatattgcattggttcactggaacacctaagcagcttgaaagggcaattgatttaggatgctggttctcagtggggcctgct |
| | | atgctcgatacaataaagggtaaggccttagttttgaaaatacccaaatcacgcattcttacagaaacagatgggccatttgctaa |
| | | gtttcgtaatgacccactaatgccatgggatagtgggattgcagagaaacagttagccgcattatgggggattagtcagatggagg |
| | | ttaatgctcagctagttgataattttaaggtattatgtacatcataagaatgaaaaacttagatatgcatttacagttcaattcat |
| | | ttttcgtcatcagttaattacacataaaattaaaagtaagaatatatctaccctgtgaatgagcaaggcggatttatatagtttgt |
| | | aattagtttaaatgtaagcagttcgtcagagtgcgtattccgctctattcgatcacggattggccgttatgaccc |
| | | (SEQ ID NO: 31) |
| 35 | pLG037 | gaaattatttggaatggatgatggcgcttgattactggaacaggtctatgacatgaaggttatgatttgttcactgctatgaggtt |
| | | aacactttaacaatttcccttactattcttgtactaattccttccaaatacttctgcttgagattaggatttatcctcttgtagtg |
| | | ttatttacaataaagattgtgatgctgatttaacccaacgtgttgtcagttgccttgctgaactaagttcagtatctagaaattag |
| | | ctcttgatacatgagcgaatcagcgaaaattttcatcccgaccaattaatgaccgtaatggataggatgttgctgctatttggctt |
| | | ccatgagggaacatatgtttttaaacgatcaagaaacgtccactgacctgctgtactacaccgctatcgccagcacagtggttagg |
| | | cttgttgatgaaacgtcagatgcacccattacgattggtgtgcatggtgattggggggcgggaaaatcaagcgtactaaaaatgct |
| | | tgaggctgcctgcgagaaaaaggataaaacgcactgtatctggtttaacggatggacgtttgagggattcgaagatgctaaaactg |
| | | taatcatcgaaaccatcgtcgaggatcttgttgcctcgcgcccgatgagcaccaaggtggcagaagcagcaaaaaaggttcttcgt |
| | | cgaattgactggttgaaaatggccaagaaagcggggggactggcgtttaccgcatttactggcatacccacatttgatcagattaa |
| | | ggggatgtacgaactggcatccgactttctaagtgctccgcaggacaagctttctgctgcagatttcaaagcgtttgctgaaaaag |
| | | caggaggcttcatcaaagaggccgatactgatagtaatacgctacccaaacatattcatgctttccgtgaggagttcagggcgctg |
| | | cttgatgctgctgaaattgaaaagctagtggtgatcgttgacgatcttgatcgctgcctgcctaaaaccgcgattgaaacgctcga |
| | | agctattcgccttttcttgtttgtagagaaaactgcatttgttatcggtgcagatgaagccatgatcgaatatgcggtaaaagacc |
| | | atttccccgacctgcctcaaagcaccgggccggtaagttatgcacgcaactatcttgaaaagctcatacaggttccatttcgaatc |
| | | cccgcactgggaactgcagaaacgcgtatatataccacgttgttgcttgcagaaaatgcgttgggttcggaggacgacaattttaa |
| | | agcattgctcaataaagcacgggaagagatgaagcgtccttggatcagccgcgggcttgacagagaggcagtgatggcagcgttaa |
| | | atggaaagattccggaggttgtggaaaacgcgctgctattcagcctacacgttacccctatgcttagttcggggacacatggtaat |
| | | ccaaggcagattaaacgctttttgaactcaatgatgttacgccaggcgattgctgatgaacgcgggttcggtagtgacattaagcg |
| | | tcctgtactggcaaaaattatgcttgctgagcgtttttaccccagcgtatacggaaagcttgttcagcttgtatctaatcatccag |
| | | agggaaaaccggaagctttggcggagtttgaagccttggtcagaggggggaaaactgctccgaagagtcgcgctgacagcaaagag |
| | | aattcctcagagtctgaagacgtccaaaactggctgaagattgattgggcgatcggttgggcaaaagcagagcccgcactttctgg |
| | | agaggatcttcgtccatatgtgtttgtcactcgtgacaaacacagtactttgagtaatctggtcgtatcaagccatctcattccta |
| | | taatggagaaacttcttggtccgaaaattgggatggtgaaaatcaaaggggatttagagaaactgagtccaccggatgctgatgaa |
| | | ttattcgaaatgcttagcgataagcttttccaagaagacagtttcaatcgaaaaccaagaggatttgacggcctcgaatatctcgt |
| | | agaaacacaacctcaccttcaaaggagattgattgattttgcacggcgcattcctgtaaaaaaagcagggggatggcttgctaccc |
| | | gtattgcgcaaagcctagtggaccctacgttaatagaagaatatacaaaactgatccaagaatgggcgagtcaggacgaaaatctg |
| | | tccctctctaaatcagcaaaagcaaccctccagttatcgggatatcaacattaatgggaacctcaaaagcttacggggggcctgtt |
| | | catggcctaatccccgatttcgtggagaatccatctccaccgaccctgccgcctgttgaccctgcggatgatagcacgctggatac |
| | | gccgctcattccaccggattcgagtggctcagggccacttagcacaccgaaagcaaactttactcgatactcccgttcaggaagtc |
| | | gtagttctctgggtaaggcggtcgctggatatgtccgcaatggagtggggggcgcaggcagggccagccgccgtatgggggcctca |
| | | cgcgctgcagcagggggactgctcggtctcatcagcgactatcagcagggaggtgctactcaggctcttgagcgcttcaatcttgg |
| | | taatttggcagggcagtctgcatcgactgctcttctctcccttgttgaatttttatgccctccaggtggttctgttgacgaggggg |
| | | ttgcgcggcaggctatgctagagaccatcgccgatatgtctgatgtaggagaggagaattttgatgagctcactcccgatcaatta |
| | | aaagaagtctttattggtttcgtggttcactccattgaagggaggctcatggcggatattggtaaaaatgggatcaagttaccaga |
| | | cgacatagacgctatcgtcagtatccaggaggacctgcatgattttgttgatggagctactcgtacacagctccgtgaggagctga |
| | | ggaatcttacagggctttcaggggatgctatagacagaaaagtggaggagatttacaccgtggcatttgaattacttgcccgagaa |
| | | ggggagagattggaatgagccatcataccttagttgcccgtttgggcactgacgataactccgatttacagctcagccgccaaagc |
| | | acgcatctgacagaaattaattttctcaaagagaacggtaaactggatttcggtctcgggcaggcgctgaatggtttgagtgatct |
| | | tggtttaacgccaatggatgtctccgtggatctggcactactggccgcaacggtgactgcggcggacacccgaatctcacgtgggc |
| | | ataacgctcaagatctgtggacgcgcgaaattgcactttatatcccggtagcttccccgacattatggaatagtcagactggattg |
| | | ctcagcaggatgttgaattttcttaccggcgaccgttggacaattcatttccgctcgcgccctgttattgagcacgggctcattca |
| | | gcgatcctctaaggaacgttcggtgaaccctacttctgtttgcttgttttccggggggctcgacagcttcatcggtgccattgatt |
| | | tattatctaatgggggaaccccccttctgatcagccactactgggatacgactaccagcgtttatcagcagaagtgtgctcagctg |
| | | ctgtcggagcgatatggacaatcgttcagccatgtgcgagctcgtgttgggtttgaaaaaacaacgattgagggagaagatggaga |
| | | aaacacccttcgtggccgctctttcatgtttttctcgctcgcgacaatggccgcagacgccctcggcgggccggtcacgataaacg |
| | | tccctgaaaatggtttgatctctctcaacgttcccctcgatccgcttcgtgtcggagcgctaagtactcggacaacccatccgttt |
| | | tacatggcgcgttttaatgagctgctgggcaaccttggcatcagtgcacatctggaaaatccctacgcctacaaaaccaaaggtga |
| | | gatggctatccattgccatgaccatgcttttctaaggcaacacgcggctgacaccatgtcatgttcgtctccgcaaagtacgcgtt |
| | | ggaaccctgcgctgaatgagcagcaatcaacacactgtggccgatgtgttccatgcttaatcaggcgagcatcattgtttacagct |
| | | ttcggcacggacgatacgatttaccgtatcccggatctccgtagccgggtactggacagctctaagcctgaaggtgaacacgttcg |
| | | ggcatttcaatttgctctggcaagattggcgcgatcaccgagtcgagcaaaatttgatattcacaaaccagggccgctcagcgact |
| | | atcccgactgcttagctgagtatgaaggtgtttatctgagaggaatgaaagaagttgaacgcctgctgagtggagtcataacgagg |
| | | ccccttacatgaaattagcaggacagaagcccgctccacaatgggtcgattttcactgtcatctggatctataccccaatcactct |
| | | gcactcatccgtgaatgtgacatttcacgtgttgccacgctagcggtgacgacaacccccaaggcatggatgcgtaaccgggagtt |
| | | aacttccgattctccttatgttcgtgtcgcacttggtctacatccccagctgattgcggaacgtgagcatgagatagcgttactgg |
| | | agcactatctcccttctgcacgttacgttggggagatagggcttgatgccagcccgcgcttttatcgcagctttgaagcacaggag |
| | | cggattttttcccgtattctgaatgcctgtttcgagcagggggataagattctcagcatccacagcgttcgcgctgcagccaaagt |
| | | gttgggacatttggaaaacaccagacttactgaaaattgcaaggctgtcctacactggttcactgggagtatctccgaggctcgac |
| | | gagctgttgaacttggatgctatttctctattaatgaagagatgctacgttctcctaaacatcgaaagctggtgtcctttttgcct |
| | | ttcgaacgtatcttgacggagaccgatggaccttttgtgtttcacgaagaaaaagcgatacaccctcgtgatgtgcagcgtacggt |
| | | tcatgaaatcgcgcagatccaccacgtatcggacacagatgctgctatgagaatactttataatcttcgaagtttagtcaccaata |
| | | gttctcacagtgagaatagttcatgaatctaattagttggattaatacaggggaatagttgaatacttcagtcccctaaaagctaa |
| | | tatgctctatgtcatctaatgataagtggctccaaagagccacttatcattaacttttctaaagggaggtagaagt |
| | | (SEQ ID NO: 32) |
| 36 | pLG038 | ttaatgcaaacgcatcaggaagggcagacctagtcacatgtagaatacgatagcaataaaaaagtctaattagaatgcaaattgat |
| | | gcaactctatgccctccaagaactccaaacctgaaagatttatgtaaaacatagtgttcgtttcaccaaaatacatataaactaca |
| | | ttaaaatagaaatttgtctcacctataagccatttagacaacagattaatgaggtttgtatcacaaatgaccacaaacgagatact |
| | | ttcgcagcttatcagtcttggactcaaaggggataaagttgcttttgttcggcaggcttcgaaactcgcgcgttcctatgattcta |
| | | tggggctgcctgagcttgcttcagccattagaggtagtattcaagataaaaacacgtttaacttgcagaaagtatcacgcagtaca |
| | | tcacctatttttgaacgtcttgatacattacctgtagataaagaaactaaatttgatttagcagacgtaactcaaccgtcttctga |
| | | aattcaactcccattgttgaaagatagcactctgaaaaaaattaaagaatttttgactttcactgaacgagctaaagaattaaagg |
| | | atgccggtcttggcgtgacatcctctatgattttatatgggccaccaggttgtggtaaaaccttgacatcaaaatatattgcatcc |
| | | tgtctaaatttaccgcttcttactgcaagatgtgactccttagtctcatcatatctggggtctacttctaaaaatatcaggcagct |
| | | atttgagtatgcaagtaaagcaccatgtgttttatttctagatgaactagattctctagcaaaggctagagatgatcagcatgagt |
| | | taggtgaactgaagagggtggtggtttctttattgcaaaatattgacaatctacctgaagaaacaatattgattgctgcaagtaat |
| | | catgaaaatcttctagatagcgcagtttggaggcgctttgagtatagaatatctattggattgcctgattttgaagtcagaaaaca |
| | | actatttgaacaatattcaaacataaaagctacatatgacgattttgttgatgaccttgcggaaatatcatcagggctaaactgct |
| | | catttatagaacaatgctgcttaagatctgagcgacatgctctggtttacaataataaacaaatcgatacccgatttttagtcgag |
| | | gctatcttagaagcgaagggagttacatttgatgaagaagataatttacttataaagattgtgaccactctcagagaatacaatcc |
| | | caaaagatttacaatacgaaagatagcaaaaatactagggctttcaaatgctaaagtgtcaaggctaactaagaactatagagaga |
| | | tattatgagtaacaaagaaagaccaataaaaataattgaggcgacacctcaagattttactgaaaaaacatataatttcggaaaga |
| | | aacaacctatccgaacagtaacaactagtctaaaaaatagactcaaacaagaagtcgatgacgttaaaaattttttccagagctca |
| | | tttaaaaaatggcccaatataccggcggtggctagagttactcttcatgaaaaagctcttgctaagtcacatcgcccatcaagcct |
| | | attaggtgataatacatgtcccgtaataggcagtgataattttggagaattacttataagtgttactgaaaaagggttagcacaac |
| | | ttcgcaaaaaaattgaaaatagcactaattctcataatgggacagtacatattgctgtaattgaaaagatcgaaccttttagtctt |
| | | aaccatgatgttatagataaaaataaatcagatagttttcttctgaaactctttgaccataaagatagaacaactaaccgcagtat |
| | | cgacaaagaattaatggaatttgcagatgaactaggaatacaaaaacccaaaaagtatgatatcagttcagatttgagtatatatg |
| | | aagtaaaagggaatgataacatcgcccaactggcaagttttattggcatacgaaaattagaacctatgccaacatttggtcttact |
| | | catacagtatcgcaatatattcctgctgaaactctagacctagatgattttcccttacctcaagaggataaacattatccactact |
| | | cggaattatagatagcggagtcgatcccaataacaacatacttaggccatggatttgggatagtttagatttagtaaaaggagaac |
| | | acgactattctcatgggaacatggttgcaagtttagcaattaatggaagatggttgaataactatgctggttttcctcaatgccaa |
| | | gctgaaattgttgatgttgcagcctttcccaaagatggtacgctcaaattgccacaattaatgaaagctatccgagaggctgtgac |
| | | cacctatccagaagtacgtgtatggaatctgtcattaggttgtcaatccccatgttctgaagacagcttctctgaattggggcatt |
| | | ttttaaatgcacttcatgatgagcatgattgtcttttcgtcgtagcatccggcaactacatttatgatcctcaacgaacctggcct |
| | | cctcaagaattaggtgggcatgacagaatatcagcccccgcagattctgttcgttcattaactgttggctcagttgcccatttaga |
| | | atcgtctgactctgtggtcaaaagatttgaaccttcatctttttctagaagaggtcccggcccagcctttatacccaaaccagaga |
| | | taaatcactttggaggtaattgtgacagtaaattaaactgtgaacataccggaatcatagctattggcgaggacaatgctctttgc |
| | | gaaagtattggcacaagtttatcagcaccgttaatctcaagtttagcggcatcactgtggcatgaactagatgttaatggttctat |
| | | ttcaccatcgcctgaacgtatcaaggcactattaattcattctgcgttaaaaaactcaccagccaaaacggagcattatgcgttta |
| | | attatcaaggatttggacgcccaagcgatcatataaatgatattattggttgcaataaaaatgagattacatttctatttgaaata |
| | | gatacccgagaaggtattgaattcagtagaacgccatttgtaataccacagtcattacgtactgaggatggaaaattcacaggtga |
| | | aattattatgacactcgtttattctccaccgcttgattatgactacccatctgaatattgccgttctaatgtggatgtgtcattcg |
| | | ggacttacacttatgatccagttaacgctaaatggatacatagcggaaaaattccacaaataaaagaaaagagtgaattatttgaa |
| | | aaggtactgatagaaaatggcttcaaatggtctccagtcaaagtttatagaaaacaatttccgcaaggtataaatggggagcaatg |
| | | gagacttaaacttgatgttcagagacgagcagagcaagagcctctatcttcacctcaacgtgctgtattggctattacgttaagat |
| | | ctcttgccaattctactacagtctacaacgaagccgaggttgaaataaataatcttggttggaaagaaactgatattgttgttcgt |
| | | gaacaaccaaaaatcaggattcgtcaaaaataagcattatggtcaccttttataggtgaccat |
| | | tta (SEQ ID NO: 33) |
| 37 | pLG039 | atagaacgatgaaggatggaagctacatattctcggtactaagatttatttttctgacacaaaatgaccatttggcgttacataat |
| | | cccaaaaaaacgtatcaaaaatctcaaaatgcgttacgattagagagtattttgattctgcgtgctcattttttgattgctgtggc |
| | | tttttgttgtgggagtgttgaatggattatttatcagaagtgttaaaaatcattgaaggtgcaacaaaggcaaatgcttcgatggc |
| | | tagtaattatgctgggttgctggcagataagctcgaacaaaaaggggaggtcaagcaagccagaatgataagagaaaggttgctta |
| | | gagctccccaggcgttggcaggagctcaaagggctggaggtgggatatctctgggctcattaccggtagatattgatagtcgactc |
| | | aacactgttgatgtcagttatcctaaattagacagttcagagatttttctgcctgcagcaatcagtacccgtgttgaagagtttat |
| | | cactaatgttcaacgttatgatgagtttgttaaagctgatgcagcattgccgagtcgtatgctcgtgtatggaaagccaggaacag |
| | | gtaagactatgttatctaagtacatcgctacccgcttagattttccacttcttacagtgcgttgcgatactttgattagtagttta |
| | | ttgggacaaaccagcaaaaatcttagacaggttttcgattatgtaatgcagaggccatcagtgctttttttagacgaatttgatgc |
| | | tttagctggagcaagaggtaatgagagagatataggtgagcttcagcgagttgtcatttcactattgcagaatatggatgcggcat |
| | | cagaggatacggtaattattgcctcaactaaccatgagcaacttctggatcctgcaatctggaggcgatttagcttcagaattcca |
| | | atgcctctgcctgacatacatcagagagagttaatttggaaaaatcgtttaaagaatatgatatgtagcgatctagatttaagtga |
| | | tttatcaagaaaatcggaaggattatccggagcaataattgaacaggtgagcttggatgcacgtagggatgcagttattgaaggtg |
| | | caagtgtgataaatcaccataaattgtataggcgtttgtatcttgctcaatcgcttatggaaggtgtaaatttaagcacttacgaa |
| | | gatgaaattcgttggttacgttctaaagataaaaaattattttctatcagagttcttgctaatttgtacaaacttacatcaagagt |
| | | aatttcaaacattctgaaggagtcaggagcatatgagcagaaggggtacacagtttagtaacgcaaaagttacaaacccaatgtta |
| | | agaatccctttttccagtagtgacttgggtgcaatagtaaacgctggcggtggggcaaaggtattggttgatgtaacagccgaata |
| | | tagacaagggctagtaagaaatttaacaaccagtaaacattatttagaatccaaactttcagagtaccctggaagcttgggtactt |
| | | tggttttcaaattaagagaccagggaatagccaaaacgcataggccgaacaaaattgctcaagaggctggattgcaaaatgccggt |
| | | catgccaaaatagatgaaatgttggttgctgctcatgccggctgttttgacgtattagagtcagtcattttacatcggaatattaa |
| | | agcgattttggctaatctaagcgcgattgagcgcattgaaccttgggatgagaataggaaggttccaggaggcactgatggtttgt |
| | | ttgaatcatcaaacatccttgtacgactatttgagtacacaggtgaagatgcaacttacaacaactatgaaaacgttatttctata |
| | | ttagaacaacacggagttaaatatgatgagattagacaaaaatgtggtcttcccttattaaggataatggatttatccccaaatga |
| | | tagatatatattagacattctcattgattacccgggtataagaacgttaattcctgaaccaaaatattcagcattcccggttagtg |
| | | taagtgattctgttggcattgaaacaaatagctttcccgtaccatcagaagaattacccattgttgctgtatttgacactggggta |
| | | agccccatcgcggcaacaattactccttgggtagtgagtagggaaacatacgtaattcctcctgatacgagttatgaacatgggac |
| | | tatggtgtcttcattgatatcaggcgctcattttttaaatgacaatcatccatggattcctgatacaaaatctaaaatccatgatg |
| | | tttgtgccttagatgaaaatggatcttatatatcagatttaattctgaggctagcagatgctgtaaataaaagaccagatataaaa |
| | | gtctggaatttgtctttgggaggcggaccatgtaatgagcagacgtttagtgattttgcgatggagttagatcggctcagcgataa |
| | | atttggtattttgtttgtagttgctgcaggtaattatgtagatgaacctatacgtacatggccaaatcctgatccgcttggaggtg |
| | | ctgatttaatttcctctcctggagagtcagtccgagcactaacagttggttcagtttctcatatggaagctaatgatgctttaagt |
| | | gaaattggaacaccgacaccatatactcgtcgtggccctgggcctgtatttactccaaagccagatataatccatgctggcggtgg |
| | | ggttcatagaccttggaatgtaggagcaagcagtttaaaggtcgtagggccagataataggctttgctctaattttggtactagtt |
| | | ttgctgctccaattgtggcaagtttagctgcgcatacatggcagagaatagccactaatacagactttaatgtttcaccatcattg |
| | | attaaagcattattaattcattccgctcaattatcttctcctgattactcgccaagtgaaagacgctatttgggagcgggaattcc |
| | | taatgaagttattgagaccttatatgatagtgatgataggtttactctgattttccaaacattcttggttcctggggtgaggtgga |
| | | gaaaggataactatcccataccatcggcacttattcaaaatggaaaatttaaaggtgagattgtaattactgctgcatatgcacca |
| | | ccactgaaccctaatgccggcagtgaatatgttcgcgcgaacgtagagctaagttttggcttaattgagaataatactataaaagg |
| | | aaaagtgcctatggaaggagaaaacggtcaatctggatatgagagagctcaaattgagcatggtggaaagtggtcaccagtaaaaa |
| | | ttcatcgcaaggcatttaataaaggaattacttcgggtaactgggctcttcaggctaaaacaacgttgagagcgaatgaaccggcc |
| | | ttaatggagcctttacctgtaactattgtagtaactttaaaatcattagatggaaacacacaagtttatgctgatggcgtaagagc |
| | | tttaaatgctaataactgggctcactatccattgcctgctcgtgtgccagtttccgtataacaactatataaatcaaacccgctgt |
| | | agcgggtttgatttatttgtgggtgtgttttataaaaataccgcccatacacaacaaaatacaa (SEQ ID NO: 34) |
| 38 | pLG040 | gggacactcaggttacataacaatgagtgatacagttcacgtagtgaaggtactatgcctaggtgtttgattacactttgatcatt |
| | | gatgatacgctcatgaaggtattactttcctgtaatgagcaggtaggtaacgatgtcgaactaaatgaatttatagtaaactttgc |
| | | aacaagagaacaagggagtatgaggggttatggctactgcagagcagatcaaagctttattgaaaagccacgttgatcgtgatgat |
| | | cagcgtttcttttctattgctttgcaggtggcagctaaggaagcaaggcaaggtcatcataagcttgctaatgatataaaaaactt |
| | | agttgataaaaatcagaaaacaacgagttctgtaggtttagttgaaaaacgacttacaccatttgttaagcagcctgatggtgatc |
| | | ttaaggggttacttgagcaaacgaacaagccagtacatcttcaagatctggtgatttctggaagcgttagggaaagattgaatcag |
| | | gttctgcttgaacaaaaacagaaagataaactttctgagtttgggcttattccaagaagaaaaattcttttcactggtcctcccgg |
| | | tactggtaagacaatgtccgcatcagtcattgctacagagttaaagctaccactttatacagtcgtcttagataatctaatcactc |
| | | gctatatgggtgaaactgcagctaagctgcgtttaatttttgaccacatacggcaaacaagagctgtatatttttttgacgagttc |
| | | gatgctataggaactcagcgtggcgctcagaatgacgttggagaaattcgtagggtcttaaattcttttttaatgtttgtagagca |
| | | ggatgattctgagagcatagttttagctgcaaccaatcatccagagcttttagatcgcgccttatatagacgatttgacgatatta |
| | | taccgttcacaaggcctgaggataatctaatcaggaatcttattgaacagagactcgctgtctttgacctcggtaatttattttgg |
| | | agtgagatcattgatagtgcttcaggtctaagtgcagcggagatcacgcgagcaagtgaagatgctgccaaagaatcagtgcttta |
| | | taatgcaaacaatattacaaccgatttgttagtaaaggctataaagcgtaggcaagaaagtagacaataagggatgaaatgactac |
| | | caacaagaggcatattttattaaacggctatgtttcccccgaaaactatcgctctaggagcaatggtcgtagtccccaagtcccag |
| | | ctcgtgatcgagcggtacatggtatatcattactaaatcagtatagccgtatattgaatcattatgatgaaagaccgaggcttccc |
| | | cctgttactgatgaaaaagggatttatgttaggctaatcagttttgaacaatgcgatcttcctatagataaaatcgataatactta |
| | | tttcaagctttgttctttagttaaatcaaataatcgtgaaactgcgattatatacattaatgaaaatgacagaactaaattcacta |
| | | aaaaaataaatgactatttgaatccatcgaaggatggtatcgagttccctagaaatcatttgttaattgatagcatacaaaatatc |
| | | gagttagcagatataacttctttctggacagataaaaaagatcttattccggatgatcacggtgttgaaaagtggtttgagctttg |
| | | gcttaagggtaataaggaggatgtgctaaatattgctcggcgtttatgcgaaagaattaatggaaggctcgggaatacttctatta |
| | | attttttcgatactactgttgttcttatccgtacgagtctatcgagattaaaagtttgtcctgaattaatatctaatttaaaagag |
| | | ataagatcagcgagggatgatatatcagttatagttaattccttacctacagaacagcatcagtgggcagaaaatgttgctgcaag |
| | | aattacgcgtaacaatgaagctgatgtttctgtttgtatattagatacaggtgttaactacaataatccactattatctagattta |
| | | ctaactcatcactggcagctgcttgggacatatcttggccacttttcgatgattataatcaaaggccttataatgaccacggttcc |
| | | agacaagcaggactatgtgtttatggagatttcctgtctgttttattgaacgatcaggacatttcgattccgtacaatatcgaatc |
| | | aggaaggatactacctccaagagctactaatgatcctaatctttatggagctattactacaggaacgtcaagtcgtctggagctgg |
| | | aaaacccgaactggcgcagagtttattcgcttgctgtgacagcagagcctaatactcttggaggccaaccgtcctcatggtctgca |
| | | gagattgacaagtttagttttggtttagaggatgatatccgcagattatttataatttctgcgggtaactctcaacctacaaattt |
| | | agaattagattattgggattcagtgactcttgctgaaattgaagatcctgctcaatcttggaatgcattaactgtaggggcgtata |
| | | ctgataaaacaacccatacagaccgcgaatatgatggttggtctcctttcgctatgtcagaagatattgcaccgtcatctcggtca |
| | | tcggtatcctggggatggaaaaagcatgccccatataagccagatttagtagaggaaggcggaaacaaacttatatcacctagccg |
| | | tgatgaaatcacaaatacaattgaattatctttgctcacaacctctggcagggcaacaaatcaattgtttgaagttaattcagata |
| | | ctagcgcagcctgtgctctagtatcaaaacatgctgctatgctaatggctcagtacccagaatattggcctgaaactattagggga |
| | | ttacttgttcatacagcaagatggactagtcgtatgcacgaacgatatagaacagaacgtgcacaggggacaccaaaatcggctaa |
| | | agaaagcttattaaggatggttggttatggagtacctaatttaaatcgagcaatgcatagtgcggaaaatgcacttacattaatat |
| | | ctcagtcggaaatcaccccatttaaaagagatggttctactgatcctacattgaatgaaatgcatctgttttcactcccttggccc |
| | | gtagaagctcttcgcttactaccaccagaaacaaatgttattttaagaatcacattgtcgtattttattgaacctaatccaagtca |
| | | aaaaggattcagacgacaatattcgtatcaatctcatggattgagatttgcagttattagacctaatcagacccttgaaaatttcc |
| | | gtgcttcgataaaccgtaatgcgaataatgaagaatacaatggacctgaaggagatgcgtcaggatggtttctggggcctcaactc |
| | | agagttagaggttcattacactcagatgcttggaaaggcagtgctgcagatttaacagagatgaatactatcgctgtctatcctgt |
| | | tggtggatggtggaaatatcgtactgcgcaggatcgctatattaacaatgttaaatatagtttattggttagcatagatgtaccag |
| | | atgagaacattgatatttacagtgagattcaaaacattattcaaattgataatcaaatagatattgaacattaaggttttatgcct |
| | | aaggtttaatgagtttgaaatgaaaaatcctttactaattggctgggtcgatgataaagacctggccatctttttatacggaaatg |
| | | atttatgttttattttactaaatttatattagaaccatcgtgcagattgtgataattccttcatactgattttttacctattatag |
| | | ttgatttttgttgcttgatatctctctttaatacaacggcgtagtac (SEQ ID NO: 35) |
| 39 | pLG041 | cggattgaatctgtttatgaaatttggctgctatcaactaatgggcgttaagttgattgtatgatctgattgataaagaaggggct |
| | | aaaaatctcctcttctttgcagcagtttactgcggtctttttgtgatgcatcagcataaaacgttttacttgtggaccctaagaaa |
| | | tggagaacattatgtcgactgtagatacctctacagcagaggaactcaatcaaggaggctcagattttattctgacttccctcgag |
| | | gctatgcgtaagaagttattggaccttacgtctcgaaatcgacttttgaatttccctatcactcaaaaagggtcttcactacgtat |
| | | tgttgatgaattaccagaacagctttatgaaaccctttgctcggaaatcccgatggaatttgctcctgtgcccgatccaactagag |
| | | cgcagctgttagagcatggctatctcaaagttgggccagatggtaaagatatacagttaagagctcatcctagcgctaaggattgg |
| | | gcgcacgtcttaggaatccgtacagattttgatttaccagatagccataaaacggttgtttctgattcagatagagagttgctgga |
| | | aaaagcccatcagtttatcttgcaatatgcccaaggccagaatggaaaattaacagggattcgttctgaatacgttaatcaaggta |
| | | tagctttgtcagcgttgaaggaggcgtgctgcttagcaggctatgaagggcttgaggattttgaacgacaggcaaaggctgggaat |
| | | gagattagtatatcttcttccaatccctctcatgacgataatcggatacaggctctgctttatccaaatgaactggaagcttgttt |
| | | gcgcgccatctatggtaaggctcaaactgctttggaggagagtggcgccaacatcttgtatttggcgttagggttccttgagtggt |
| | | atgaaagcgattcctctgaaaaggcacgttatgcaccgttatttacaattccggtgagatgtgaacgaggaaaattagatccgaag |
| | | gatggtctttacaagtttcaactttattacacgggtgaagatattttgcccaatctctctttgaaggaaaaacttcaggctgactt |
| | | tggcctcgctcttcctttgttcaatgaagaggaaactccagagtcttattttgcttcggtgaagaaggttgtagagcagcacaaac |
| | | ctaaatggtctgtgaaacgttatggtgcacttagcttgctcaattttggcaagatgatgatgtatcttgacctcgatcctgcccgc |
| | | tggccttgtgacaagcgcaatatattgtctcatgaagtaattcgtcgctttttcaccagtcagagctgtggtcaagagaattccgg |
| | | cttacctggtggcttcggtcagcatgagtactgcatcgatagttaccctgatattcatgacaaggttccactaatcgatgatgcgg |
| | | atagctcgcagcacagtgcgttgatcgatgctatccgtggtcaaaacttagtcattgagggccctcctggtagtggcaaatcacaa |
| | | acgatcaccaacttgattgcagcagctctgctcaacggtaagaaagtcctgtttgtggcagagaagatggctgcactggaggttgt |
| | | caaacgtcgcttggatcgtgcggggctaggtcaattttgcttagagttgcacagtcataaaactcataagcgcaaggtgctggatg |
| | | atattaatgctcgcttggtgagtcaggcgaccatgcctactatggaagagattgatgctcagattttgcgttatgaagatcttaag |
| | | cagcagctcaatgaatatgccgcattgatcaataaccaatgggcgcaaacaggcaaaacgatccatcagattttgagtggtgcaac |
| | | ccgttatcgtcacaaattagatattgatgcaacagcacttcatatcgaaaacctttccgggaagcagttggataaagtgacccaat |
| | | tacggctgcgtgaccaaatagtagaatttagccgcatctacaaagaggttcgtgagcaggtgggggctaatgcagaaatatatgag |
| | | cacccttggagcggtgtgaataacacacaaattcaattgtttgacagcgctcgtatagtcgatttgctacaaacttggcagacatc |
| | | aattatcgactttcaacatagctatcaagaatatgtagataagtgggcgttagaaggcgaaagccttaatacgcttcaatatattg |
| | | agcaatfggtagaagatcagtcgaatcttccagtgttgtgtggttcagagcatttcccagcacttagtgagctagattcacccgat |
| | | gccattgcacgggtgcgtcactatttagataggttcgagttgctacaaggtcattatgtggccttgagccaggttatcgagcctca |
| | | aaagctacgacttttagaacaaggacaatcgtgtgactttcctcgtgaagagctggaaaaatatggtgcagcagaggatttcactt |
| | | tacgtgatttggtcaggtggcttgaatccatccaatcaattcatgatgagttatcatctatttatgcgcaattaaacgatttcaaa |
| | | aatgctttgccagatggtattgcttcgtatatcgatgattcgcaagctggattgctattctgctctgagttgttgtcgattctggg |
| | | tgctttaccgactgagcttattagagttcgagatcctctttttgatgatgatgatatcgatgcagtattgcgcgacttaatgtgtc |
| | | aaatcgaaacattgcgtcctttaagagatggtctatctactttgtatcaattggaccagttgccttcccaagagatgctcgcgcat |
| | | gccgttgctgttatccagcaagggggattatttgcatggtttaagagtgattggcgtagtgccaaggcactgctcatggcgcaatc |
| | | tcgaaagcctgacactaagtttgctgagttaaaacgctgctcagctgatttgctcaagtattcggagctgttacaacggtttgaac |
| | | aaagtgactttggtaatcaacttggtaatgcattccgagggttggacaccgactgtgaacaactcatgttattgcgtgattggtac |
| | | aagaaggtccgagcttgttacgggataggttttggaaagcgagttgcgataggctctggattatttaacctagatggtgagattat |
| | | caaaggtgtgcatttaatcgagaaatcgcagattagctcaagattaatgactttggttaaacgggtcgagcacgaggctaagttat |
| | | taccgcgtatttctagcttgttggaagaacatgcatcttggttaggtgagcaaggtgtattgatgcaatcttaccgacaggtgcgg |
| | | aatactctcattgccttgcagggatggtttatcaatccagatatatcattagagcagatgactcattcctccgagattttgcaaaa |
| | | cataaacgatcttcagatatcccttgaaaatgactcgttacagttaggggcgtttttacaattaaccccattggcttgcggtgcgt |
| | | ataaaaataatcaactgacgttagacactattaacgacacgctgaattttgccgagcaactggttgataagataaattgcgtatcc |
| | | ttggctacccagatcagacatttggctagtggtagtgattacgatttactatgtcgtgatggtggagaaatagtttcgaaatggaa |
| | | tgaacagattaaaaatgctgagttatatgcgctagaaacaaagttagagcggagtcagtggctcaagtcgactgatggttctctta |
| | | atacattaatcgagcgcaacgaaagagcaatacagcaaccccgttggttgaacgggtgggttaactttattcgttgttacgagcag |
| | | atgcatgaaaatggattgcagcgaatctggagtgctgtacttgcgggctcgctcccgattgaaaaagttgaattgggtttagcatt |
| | | agcaattcatgaccagctggcgcgggaggttattcacatccaccctgaattgatgagagtttccggctcacagcgcaatgctttgc |
| | | agaagtcatttaaagagtacgacaaaaaactgattgaattacaacgtcagcggattgcagcaaaaattgcttgccgaaatatacca |
| | | gaagggaattctggtggtaagaaaagtgaatatacagaactagctttgatcaaaaatgagttgggtaaaaaaaccagacatattcc |
| | | aattaggcaattggttaaccgtgcatgtaatgcgctggttgcaattaaaccttgtttcatgatggggccaatgtcagcagctcatt |
| | | acctagaacctggacgaatggaatttgatctggtggtgatggacgaagcgtctcaggtgaagccagaggatgcattgggtgtcatc |
| | | gcgaggggcaagcaactagtggtcgttggtgacccgaaacagctaccaccaaccagtttctttgatcgaagtgccgacggagaaga |
| | | tgacgatgatgccgcggctttaagtgatactgacagcattttggatgctgctttgccactgtttcctatgagacgtttgcgttggc |
| | | actatcgttcacgacatgaaaagttgattgcatactctaaccgccatttttataacagtgatttggtgatattcccttccccaaat |
| | | gctgagtctccagagtatgggattaaatttacctatgtgtcaaaaggtcggttctccaatcaacacaatattgaagaagcccaagc |
| | | agttgctgaggccgtacttcatcatgcgcatcaccggccgggtgagtcactcggggtagtggccatgagttccaagcaacgcgatc |
| | | aaattgagcgcgctatcgatgaattgcgccgaaatcgccctgaatttaacgatgcaatcgatggcttacatgccatggaagagcca |
| | | ctttttgtgaaaaaccttgagaacgttcaaggggatgagcgtgatgtaatctttatttcctttacctatggaccttctgagcatgg |
| | | tggaaaggtttatcaacgctttggacctatcaattccgatgttggctggcgtcgcttgaatgtgcttttcactcgatcaaaaaaac |
| | | ggatgcatgtgtttagttcaatgcgttctgaagatgtattgacgagtgaaaccagtaaacttggtgttatttcgttgaaaggtttt |
| | | ttacagtttgccgaaagtggcaaactagattccctcacaacgcataccggcagggctccagatagtgactttgaggttgctgtaat |
| | | ggaagcactcaatcacgctgggtttgagtgtgaacctcaggtaggggttgcaggattctttattgatctagctgtgaaagatccag |
| | | gttgtcctggccgttatttaatgggcatagagtgtgatggtgcggcttatcactcagctaaatctgctcgtgatcgtgaccgtttg |
| | | cgtcaagaggttctggagcgtttgggttggagaattagccgcatttggtccactgattggttcagtaatcctgatgaggttctatc |
| | | tccgattatccgtaaactccatgagcttaaaacattggctccagacgttgttgtaccttcctatgaatatgtcgaaacgattgagt |
| | | caagcgctgaagtggcgtctgactcaattgattctcttatgcccaatttggggcttaaggagcaacttaagtattttgccacacat |
| | | gtcattgaggttgagcttcctaatgttgatgctgatcgtcgtttgttgcggcccgcaatgcttgaggctttgctggaacatcagcc |
| | | tttatcacgttccgagtttgttgaacgaatacctcattatctgcggcaagcaacagatgtatacgaagcacaacgctttcttgacc |
| | | gagtcttggcattaattgatggcgcagaggctgaagcgaatgatgcagcgtttgagtctgaattggcataattagttaaaggtaat |
| | | aagaacagtgacaactgtcgg (SEQ ID NO: 36) |
| 40 | pLG042 | gctatcctacctcagattactgggctgacctaatctatagatcaggttctctttatactttatgttagcgaaatactaagatgctt |
| | | cttagtgacgacctcttgacggtagaggacgcgtgcatagattttacaatcactgcctttcgccccctaacctaatccgcgaatga |
| | | tgcatcctgaacttgcgcgccagttcttatactcgccgtcagagcaatcaaattgctgatgctttctgcctgttcaaggcatctcc |
| | | tgtcgtcagcaatactgtgcatatttgattgatttcctcttaaggagaattagtttcatgggtattaaagcgcaggtgagtatcgc |
| | | gcacaagctggggttcacatcacaccaaaatgcagttccgctgttacgtgagcttatcttgcataatgagtccgaagagacatttc |
| | | aggatctgacactgcatctgaggaccgtgccagctgtgctcgaagaaaaaaaatggaatatcgatcgcctgcttcccggtacttca |
| | | cttgatatcagagatcgggatatcaaacttaatgctgaatggctagccgaactgactgaaagcgtactctgcgaagtcacgctaag |
| | | tttgcgccagggtgaggaagaactcttcattacccattacccgcttgaggcactggcgaaaaatgaatggggcggcagtgcaatga |
| | | ttgaattgctcccttcatttattattcctaatgatccggctgtggatcgtgtactcaaggcaacctctgatgtccttcgccgtgca |
| | | ggcaaggatgacgctcttaatggttatgaaagcaagtcgagaactcgtgtctgggaaattgcctcagctctctggactgctgtttg |
| | | caacctcaatatcagttatgcccttcccccagccagttttgaacgcaatggccagaaaattcgcactccaggagccattctggaag |
| | | gaaaagtcgcgacctgtctggatacaacattattatttgcttcagcactggaacagattggtctgaattcactgctaatgctcagt |
| | | gaaggtcatgcgtttgctggtgtctggttacaaccgcaggaattttcgcagctagtgacagatgacgtctctgcggtgcgcaaacg |
| | | tgtcgacctgaaagaaatggtcgtatttgagacaactctcgcgaccagagctcacccgccttcatttactcaggcatctgatgaag |
| | | cgttaaagcatcttaacgaggatgtttttcacgcagccattgattcccgtcgcgcgcgtatgcagaaaattcggccactggctctg |
| | | gggggcactcgccttgaagaccagtcggatgcctgcgaggttattttgcatgggtttgaggaagccccctatatccccgatgttga |
| | | tattgatatcgagacaactggcgaaaaagaagccggggggcggctggtacagtggcaacgaaaacttctggacttaaccacccgta |
| | | accgcctgttacacctgtctgaaagcgctaaaggcattcgtttgatctgtgcgaatccgggccatcttgaagataaactggctgaa |
| | | ggcaaacgcattcgcattgtcccgctccctgatctcgaaagcggcggccgcgatgccgaactttatcagcagctcacaaatgagaa |
| | | cctgcaggaagaatacgctcagattgcgctggaacgcggtgaagtcgtctcctcaatggaaaaataccgcctcgagtcatccctga |
| | | tcgacctctatcgaaaatcgaaaagtgatctcgaggaaggtggtgccaacactcttttcctcgctgttggcttccttaaatggaaa |
| | | aaatctgctgatgaccccaaaagttactctgctccactgatactgctgccgattcaacttgaccgtaaaagtgcactttcgggcgt |
| | | gaccatgcgtttgctggaagaagagccccgcttcaaccttacactgcttgagctgctgcataatgactttgctctgacaatcaacg |
| | | gcctcgatggtgatctacccaccgatgaaagtggtgttgatgtggatggtatctggaatatggtacggcgtgctgtacgcgacata |
| | | cccggtttcgaagtcacccgcgatgtcgtgattggcacattctcttttgccaaatatctgatgtggaaagatctcatcgaccgggc |
| | | acctcagctgatgcaaagtgcgctggtaaagtatcttatcgaacgcggccaggaaaatgccgttctggataagagcggagaagtca |
| | | tcaacgctcatgaactcgatgacaacatcaatacgcaggatcttttcttgccgttgcctgcagattcctcgcaaatcgccgctgtt |
| | | gtagcctctgcaaaaggcagggattttgttctggatggcccacccggtaccggtaagtcgcaaaccatagccaatatgatcgcgca |
| | | taaccttgcgctaggcaggcgcgtactttttgtcgctgaaaagaaagcggcgctggatgtggtctatcgtaggcttgaggcccagg |
| | | gactcggtgaattttgtctggaactgcactcgagcaaaacgtccaagatggattttctgaaacagctcgagcgggcatgggatgcg |
| | | cgtgatctactaaccaccgaggagtggaaggaagaagcggccaaggtgcagcacctgcgtgacaaactcaatgaggttgtccgttt |
| | | gctccatcggcgctggcccaatggcttaacactccatcaggcaatgggcacagttatcagggatgcaagtagcgccacgccgcact |
| | | ttagctggcctgcatcgactttgcattcttctgcagagatgacacagttcagagagatagtaaaacgtctggagctgaaccgtgat |
| | | gcatggaaacagcacggcgatcattttgaactcatcgcgcaggctgactggaccaatggatggcagtcctctctcattgctgcagc |
| | | aaactcattgcctgcaaccatcgatcaccttgaagacgcgaccgaggcgttactgaaggcgacgggagttactctgctctctaccg |
| | | agccggagagactgtcgcagttaacttcattctgtgaattattgtcggaagcttacggcattgatctgagtttcatgttcgcaccg |
| | | gatgccgcaagccgtatagagtcagcgaataaagccgttcacctcctgaaagagattgaagcgacaaaggctaatctgtcagttac |
| | | ctacccttgtaacagttggcagcacgttaatgtcccacagatcagaaacgcacttgacgtcgctgacaaaaaattctggttctttg |
| | | cgaccagtgcccgcaagaaagtcattggtgaagttatccgacaacactcgctaacgtcagcccccgacttatccgttgatctcccc |
| | | attgctgaaactctgcagacattgctgcaacgtctgaccgagcttaactctgctactgtatctctgccgggatgggttggactgga |
| | | taccaacgttgcacagttgcagaccaccctgcaacttgccgaatctatccgcaattcgcttggtggtttcgcttcttcgccacagc |
| | | agttggccgagatccgcactgcggtaaaaaacctgattgttgatgccaatgaccttctcggttcgcagggcgttatctccgcacta |
| | | acccggaaactgcgcacagcgatcgccgatttcaatgatgcacaggttagcttctgcaatctgataaaaccatctgaggataaacc |
| | | atcgctcccggcactgcgtgactgcgcactcaatatcctgcaacatcagtccgctcttaaagcctggagtgactggagccgtgtgc |
| | | gtgaggaagcgatttcacatggcctgcaaccagtgatcaacgcgctggtccatcttgactcaggagacatcagcgcggcagagatt |
| | | tttgaaactgcctattgccgctggtttgcatcgtggatgatcgattcagagccgctgctgcacaattttgtgccggctgagcacat |
| | | gagtgatattgaggcttaccgtacgcaaaccgatcgtctgtccaaactggcagtacgctacatccgtgcccgtttatgtggcgtca |
| | | ttcctgcaaaaaatgaggtcagcaagcagggtggttttgctctgcttaaacatgaactacagaaatcccgtcgtcataaaccggta |
| | | cgtcagatggcagcagaaatgggagatgccatggccaaacttgccccctgcatgcttatgagtccgctttcagtcgcccagttcct |
| | | gccctcggaccaggacttgtttgaccttgtgattttcgatgaagcatcgcagattgccccgtgggatgctatcggcaccatggcgc |
| | | gtggcaaacaggtggtaatcgctggcgatccccgccaaatgccgcctaccagcttttttaatcgtgcagccaatgacactgacgat |
| | | gatactgaagaagatatggaaagcattctggatgagtgtcttgctgccggcctgtataaccacagcctgagctggcattaccggag |
| | | ccgtcatgaaagcctgattaccttctccaaccatcgctactatgacagtagcctgattacgttccccgcttcggaaacaaagcaaa |
| | | gtgctgtccagtggtgcaaggttgcaggcgtctactctaaagggaaaggacgtcataatcaggccgaggcagaagcgatcgtcgct |
| | | gaaacggtgaagcgactgactgataaagagttcgttgcatcaggcagatcgataggcattatcacgctgaataccgaacagcaaaa |
| | | gctagtcagcgatctgctggaccgtgccagacagcaacaccctgaaattgaacccttcttccagtctgaactggaagaacctgttg |
| | | tggttaaaaacctcgaaacggttcagggggatgaacgcgatttgatcatactctgcatcgggtacggcccgactgaaccgggcgca |
| | | aatacaatgtcgatgaattttggaccgcttaatcgcgagggaggctggcgccgactgaatgttgccgtcacacgtgcgcggcagga |
| | | aatgatggtcttcagctcgttcgatccttcctttatcgaccttaatcggaccaacgcccgcgcggttgctgacctcaaacacttta |
| | | ttgagtttgcccagcgcggccctgtagctcttgcccaggcagtacgtgggtctgtaggcggttatgactcaccgtttgaagaggca |
| | | gtggcaaatggcctgagaagaaaaggctggcatgttgtcccgcaaattggcgtatcccgtttccgtattgatttggggatcgttca |
| | | tccggataagcctggcgactatcttgtcggtgttgaatgtgacggcgccacttaccatagcgcagcaacagcacgcgatcgcgata |
| | | aagtccggagctccatcctgcagggcctgggctggaaattactgcgcctctggtcaacagaatggtggattgataaagaaggcgca |
| | | ctcgacaggctggatgcagcaataagtcgcctgctggaggactccagagcagcggaagccgcactgattgctgaagcagaaaaaca |
| | | aaagcagattacgccagtcatcgctcccgtaaccaatgatgtcagtgatgacatactggtttctgaaactacacctgtcgctaatg |
| | | atgcggaaatatccgcgtcagtaacccctgtcatcccgcttactgccaaagtaagcgaagatgatggtaacactgggctgaggtat |
| | | gcatctttagcttctcagaataacgacaagccagtgaatgtcggtaagtatgtcgttaacgatcttcaggaatggtgcgacaggac |
| | | agatgcagaacaattctatatcgctgaatatgatgagacacttaaaaccctcattgaagcggtggtgacaagtgaatcaccggtcc |
| | | tggatacaacgcttgtgcaacgcatcgcacgtatacacggcttcactcgcgccggcagactgatacgtgaacgcgtaatggaaatt |
| | | gtggatcaacactatcaccttgcaaccgatcactcaggtgaagacttcgtctggctgtccgcagcgcaacgtgctgactggaatgt |
| | | gtttcgtttgccagccacggataacgacattcgtcaggttgacgcgatccccagtgaggaattacgcgcactggcgctgagtattg |
| | | aaggtgacaataagatacaggaaatgacccgctcgcttggcattaaacgcctgactagtcaggcaaaaaaaaggattgaatcagta |
| | | cttgatgttgtttgaaggtcaaccgtgtggaaaacctcttttagagactaacagtctgaaatatagagtcttattcgatcatcttg |
| | | agaccgaatgtattagagtcgatttctgacacctcttatcgtggttttctgcatcaccaacatcgaccagttgggcgtaatcaagg |
| | | aggacgtctggaaaacgaatctatggtcactcccgtttttgcaacaccgattttgacaataagttggtttgcttgaatctattcgg |
| | | catcagaatggaattttttttccacgcctcgatgagttccgcgcctgatgaa (SEQ ID NO: 37) |
| 41 | pLG043 | aatcccaccctgacaaaaggcctgaaaaggtcttttgtcatttcttcacagttagagccctatcgagacgcgcaaggaagagtcgc |
| | | gccagcctgtttttacgctagcgctctgctagtgacagccagctcacagggagtgagctggcagtgtttaacgtcctaccgagggg |
| | | cgtaaattgcacacagaggttaatgatggctaaagcgcactccacgccgctcaacgatattgcgattatcgctgcgaatttaaaag |
| | | accgttataaaaatggcttccctgttctgaaagaaattgtgcaaaacgcagatgacgcacaagcgtcatcattaatctttggctgg |
| | | agccctggtattgctggggcagatcaccctttattgggcgatcccgcgcttttctttatcaataatgcgccgctgacactcgaaga |
| | | tgtagaggggatcctctccattggcattggcactaaaccgggtgatgaaaatgcggtggggaaatttgggctcggtatgaaaagcc |
| | | tgttccatctcggtgaagtatttttttaccagtcctttgactggcatactgcttcggccaaatcagacgtttttaacccctgggac |
| | | agttacagatcttcttgggccgaggtgagcgagcaggataaagttcgtattgaggatgaagtccgcgcaattacccaaaatgcgtg |
| | | tgatgattatttcgttgtctgggttccgctgcgttcagagagtatctatcaggcgcgccaggatgatgaaaactttattattgtcg |
| | | gcgaagactatcgttatgaggtgcctgattttatttcagacccgggactcggggataagctcgccagcctgttaccgctgatgaaa |
| | | accttgcaggacattgagctggtcgtgaaaacagggcaggggtatcagcgtcaaatacatatctcgctgcctgaaaaggcaactcg |
| | | cccacaatttaccaatcttaatggtgctggggaatggcaaggccacattaccgttcagcgtgctggattgccggaccctcagcaaa |
| | | aattctacgtcgggcatgaggttttgctgaatgctcctgagttttctgccctgaaatcacaacgcgcctggccattcagttattca |
| | | cgagaaggtaagaagactgcggataaagcgctgcctcatgccgctgtggtgatgctggcggagaaagtaccagaaggagaggcaac |
| | | gctggcggtggaatgggcggtgtttttacctttgggtgagcaggacaccgcgcagcatgcgcagaaacaaacattctctatttctg |
| | | gtcagtactcgtatcaaattattctgcacggttactttttcatcgatgccgggcgagtgggtatccaggggctggctacactcacc |
| | | agcgccacgccgttattcaatgccccagattctccaggccaggaacaactggttcaggaatggaaccgctgtcttgctactcaggg |
| | | aacgttgccgctattaccgaaagcgcttgcctctcttatgtcgcttattcacgccagggatgcggaaaaagcggcaatttcggatg |
| | | gtgtgcgtagagctttacgcaacaataatgcctggttccactgggtaacgttgtaccatctgtgggtatgcgaactaacgcgggat |
| | | ggaagtcagtggtgtttagttgatgcgaacactcccgttcgtcgattgcctgccacaccttcaggtgaagcgcatcgcccctggga |
| | | agtgctgcccgctctggaaagtctgggtgtaacgcaccgatttatcgatgaaacgcagcagaatatctacaacgaatttaaaagta |
| | | agtggcagttgtcggagattcaggtgttgctgcatagcgtacccgaaatggtgttcactagcttaaagcttacaaattatctcaat |
| | | caattgctgaaagaactgccgattcagtcagacagctttgtgcttgacctgattgcattgctcagaaaaacgttatttagcgtgcc |
| | | gctggttgagctctcacgtaaccaggcggcgatcggagaattgatggcgttcattcgtccgacctggcgttacaggattgccattg |
| | | accgtcaggagcaggccctgtgggaaacgcttgggcgtaccgctatggataggttgttggttcctgcttttctcgataacagtaaa |
| | | gaacctgccagcgcatctctgaattgggagacggttggcagcctgctgcaagcgatgcagaaacaggcttctgccagcgataactt |
| | | tgaaaaattggtgcgggattttattggcaagctctcatctcccgatcgtcaggagctataccgtcggtttgataccttgaaggtct |
| | | ttaaggtttcacagccaacggggatatcttacctggagacgcgctgtcacttgcttgaactaaaacaaaagcgaaggatattcaaa |
| | | cttggcgggagcgctaattttggtatgggtttaagcgcattgttgcagcaggcattgcttgaaaaagaaatcgtattgatcaccaa |
| | | tgatattaaccagaccttatttggtggttctgaatattcagaagcaaaggagtgtgacagcgaaggggttatccatctgcttgagc |
| | | ttcaccctcgtctggattcgccgacaaaacgtatcgatttactcaataaaatggctgcggacggggacaaatttagcgccggagat |
| | | cggcttgtctatcgctatctgatgcacggtaattcggatgatactggtgaagctgaattgtggaaggcgggtaaagcgcatcccgt |
| | | atgggcaaaaattctttctgatgccgattcggagcaggtcaagtggactattatttcgccagaaattgagcagaatcttggactga |
| | | ctcccggattcgagaaggcgcttaggcttgatagtgtaacgccggatcatgtgatccaccgcttcaaagaaagccttgaatatctg |
| | | gagtttgatgacttatctgcagaagatgcggaagaagttctgatgcacattggccgctctatgggcgaaacaatgtggcggcagat |
| | | ggctcttcatcgtagggaaggcaaagaggggtatatatcccttgatgatcgttgtttcttgcgtggggggcgcattgaactgccca |
| | | ctgaattgaatgacaacgtgacgttcatccaacccgccagtcagccagagatgcaggatcagcagcgcaaatatctgacaatggtg |
| | | aacgccgaacatgcggtcatgctggctttatccgggccgaacccggaacgttactgcgactttatcctgcaattgttaatgcaacc |
| | | gacgaatgatttgtcttcagagagagcattcaataacctgcgccgccaaaaatggctattgcaccgcggtgtggcgatggcaccag |
| | | aaaatattctggatattagcgcggcagactatccggagatcgcgaagctgacagaagcgacgccgctcatcgctctgcttgaggat |
| | | attgctctcccagatgaggctaactgtgcgctgagttcattggtcgtgcgaggcaaggctgcgttttacaaggcgctcactgtagc |
| | | aggtacacttccactttatgcaatcggtagcagcttacgtctcactgatacgattattcttcaggccagtgacaggtcgtacgcgt |
| | | ttgagagctttgacggttggttgctcttaattgagtgtctcaaaggtgctgagtcgcttgagggtaatgaggctatcaatgcgctg |
| | | agtttttcgcatccggttacagacaagatagttgctagctaccggcatctcgttgacagcatgaatccaacccaaagtggtgaatt |
| | | gcgtaaagcactgttaagcacgctgtgtcatacccattcagatcccgccagcgtactgcgttcaatcccgctcagaacggctgctg |
| | | atacctgggcgttagccaccaatctctgttatggcgtaacgggagcagaacgtagtgctgtcctacatgacgacgactgggcgtat |
| | | ttgtccccttggctgcaggctaatgacttgtcggtagacagtactgagtccgaagggcatctcagtcatgttgagcattctgccaa |
| | | tgtcttaagggaatactttgcgccctgggaacgctgggttccacgtaaggcaattgctgcactgctggctttgctggcggggaatc |
| | | gtaaggttcataagctatgtgagagctacctggggttgcaaagttatgccctgttcgtgaatgaactgtcgcaagacagcaaaccc |
| | | ttaactaaccatgacgctcactttgcagagttaacgctcttacagtgcattgagaaatatgcctttgccgtgaaggtttacgaaga |
| | | aaacacgttgcaggttcattctctgttccaggaacgtttgaccgtggcgctggcaactgacctggatacgatctttgtgggtcagc |
| | | acggctacgctttttataccggtcaggcaccgcaaatcttcattcgccgattttccccagaccagtatacgcctcagcaacttttg |
| | | gcgattctgaaacgcagcaccagctggctgcaggaaggtatttatctgcagaaggcaaggctagacacgctctggcaatcctttga |
| | | gcaggccgagcagttggatgtgaatatcgcgcgcgtcactatcctgaacagcattgttgagcgcctgaaaacactgggccttaaaa |
| | | actctcagcttaacgttttaatgagagcctatgagagtgagcttcactctcttgctgaaagtagtgacggcaagttgctccacagc |
| | | tcgaggctcactgaaattgtctatgacattgcaaatgctatccaggatcgccctgaactgcaggctgaaatattaacggcggtcag |
| | | aaagcgtatagaggatgctcagtatcagccatcaagcgttccttttgagctgttccagaatgccgatgatgcagtagaagagttgt |
| | | tcaagctggatagcgatgcccgtcatgagcgggtacaccagaaatttatggtgaaagagcaaaacggcggattgtcattcttcaac |
| | | tgggggagagaaattaaccgctttcagagcgtgaaaaatgagcaagtcgagaatgtacatgatggctacaaaaacgatctgaaaaa |
| | | aatgctggcgctttaccagtcggataaagagcagggcgttaccggcaagttcggtctcggcttcaaaagctgtctgctggtgtctg |
| | | atcatccttacctattgtcggggcggctggcgactaaaatagcgggtggaattgtgcccgaatcctgtgatgctgaaagttataaa |
| | | caactaaaccaactcactgaaagtgccgcgacaaatggcctgtcacctactcttgtgtatttgccactgcgccagcatatgcaagc |
| | | ggaagtggtgttaaaagattttactctgtatgcaggtttgctaagtctttatgcacgtaacttgtgccagattgtcattgatgagc |
| | | atgaatggcgctgggagcctgttcagtatgcacgtattcctggtctgtcattgggcaaggttatgctgcctaacggcaagggtgct |
| | | cagtcgccagtgcgggtggtggtttaccagactgaaatcgatgatgagcgctgccatctggttttccaggtcacgcgtaggggcct |
| | | gagaagttttgatactcatattccgcgattgtggaacttgtcgccattgatgagtgatacccggcagggctttttgattaacgctg |
| | | gatttgaggttgatattggtcgacgccagttggctattgaagctgaccgtaatcggggcattatccagaaagcgggagcaaaagtt |
| | | cattcgctgctggaattactttggtgggaaacggagcataactgggaggagctggttgttgagtgggaactgagccctgaattgac |
| | | ccatactcagttctgggaaagcttctgggacgtgatgtctacaggcattagtaacgatattaacgcgatggaaaacgaaaaattgc |
| | | tacagcagctttacgaaagcgaaaatggcatcatgagcttctatcgctcatatcccgcgctgcctaacggatttaaagagcaggct |
| | | gccggactgataacgtggagcgacagagtgcgtagcgcggatgaactggtttctcgtctggcgagttcactgattcatctccctgc |
| | | gtttcaggcattgcacagtgcacagtgcctggtggcagacacgacgggaagcaaacttaaagtcgaaagtaaactgtcgcttgaat |
| | | cattaataagctcgtcgttgccggataaacagggtgttgatatccagcatctgtcaccgcgggatgctgaaaagctggcagtcgta |
| | | tttaacgaagagttcgacaagcgactgggtgaactgacaggctggcaggacaaaattgaggctttcagaaaacagctgataaacct |
| | | gcatgtgcaaacacaagcaggctctacacgcccgattagccaaattttgctcggtaacactccttgtgccgaaaaaaatgaacgga |
| | | tgatctctgggtttgcacctaccgatgccatcatttcatcatcatattctaagcaggcctgtgaatttattgtttattgcaaacgc |
| | | agaagtcagggatatgtttttgaggatttagtcaaatgggcaaagcgcaaaggcctggcggctgataatcaaaagcggcaggcatt |
| | | ttgtcgttttctgattgaaggactggaaggggagaaactggcgggtatgctgatggaagagataccaccggactggttgcttgaac |
| | | ttaagctgcgcccaggcgccttcccggcagactggcactggagcaataatgatattgcctctctcctgcaggggcggttactgact |
| | | aacattgacagaacaaaggcatgggagcgcgagattcgggagacaccggaagaatacgaaccgttggtgacaccaggtgaagccgt |
| | | acaaaaaatacacacctggtgggagaggaaccagcaggaagagttggtgaaatacaatgctcggctctaccctgaaggctggtttg |
| | | actgggaagctttaagaaatgcctctgacgatcagcgttcacgcctggcgttattgaaactcctgtatctaggctcatgccagacc |
| | | attgggcggactcaggaagagcaacacagtgccgcaattgagtattttgaggacaaaggctggtgggaaacctttatcaaccctga |
| | | tgcagcgcagcaatggctggatgtgatggacaattatctggaggattctttgtacggagatacctaccgtatctggctgcaaatat |
| | | tgcctctgtatcgtttttcaaagcatttagattcctatcgcaaactactggatatgtcggaagcgttccttgaggatattggggat |
| | | ttgctgcgaccggcatccagtttcaatctttcgggaacgggcgtgggaactgtagtcccggagttacgtgcaactctgggtactgg |
| | | ggtgaacttcatcttccgtgaattggtgcgtaataacgtatttatcgattccagcattcatcgatattgtttctctgcgccggaac |
| | | gcgtcaggcgtctgttactggcgatggagttcgacgaaatggatgttaagcaatccactgccagtgactcgcttctgctgtggacg |
| | | tttttccgcgaacatctcggtgaggaagatgcgacctttaatcattgtttcgacataccgctgcgcattttaaccagcgaagggaa |
| | | acgctcacttcgtattgagatatttggacaggatcccctggattacgtatgaaaatgatctttcagcagggccagcaggtacgaca |
| | | tgaacgctttgggctggggacgattgaactcttgcgggaaaacactgcactcattcgtttcgagtcgagttttgaagaacgtccac |
| | | tttccgaactggagccggtgcgcagtgctcaggatgctttggcagaaggaaattatgacgatctgcgtgaagttctggcgcgcagt |
| | | caggcgcttgcgatccgctccatcaatgatagttggggggtgttctctacttcacgtatcaacctgctgccgcatcagttatgggt |
| | | atgtcaccgcgtgttacggcaatggccggtacaaaagctgattgctgatgacgtagggttggggaaaaccgttgaggcggggctaa |
| | | tcctttggccgctgctggctaaaaagcgtgtgcagcgtctgttggttttagcgcctgcatcgttagtaccgcagtggcaggagcgt |
| | | ttgcggcagatgtttgatattcgtttgtccctctactccgcggaaattgatactgagcgatcagattactggaatacgcatccctg |
| | | ggtggtcgcttcattgccgacactgcgaaaagatattaatggcaggcacgagcgaatgctcaaagcagacgactgggacttgctga |
| | | tcatcgatgaagcacatcaccttaactcgctagaagattcgggggcgactcagggctatcgatttgtgcagaagcttatcgatcac |
| | | ggaaagttcgcctcacggctttttttcacagctaccccccatcgcgggaaaaattacggcttctttgctctgttgaggcttttacg |
| | | tccagacttatttgacgtgaataagccatttgaaactcagcagcatcatgttcgggatgttgtgattcgcaataataagcaaaccg |
| | | tcacgaatatggacggtgagcgtttgttcaagaccgtcaacgtgacctcacagacctatcatttttctgaggctgaacagtcattc |
| | | tatgaccggctcacacgatttattctttcagggcaggcctacgcttcgtcgctaagctctgcaaaccagcaggccgtgcaactggt |
| | | gttaacggcaatacagaaactggcggcaagttcggtagcggcaatttatgccgcaataaatgggcgtatcgccaggctcggggaaa |
| | | atcagaaaaagctgcaggcgctgaatgatgaaatgaatgccatcatgagtgattctcaggccccggatctcgatgatgcctacatt |
| | | gcgcttgaaagcgaatatgttgaaatgtctgcttcggttcaacttatgcaaaatgagctgcccatgcttgaagagctgcaggcgct |
| | | tgcggggaatgtggaatcggaaacgaaaatccagaccttgcttcatgtgctggaaaacacgtttcttaatcgcaccgtcgtattct |
| | | ttactgaatataaagcgacacaggccctgctaattaatactctgaatgctcgctttggctatggttgcgtcagctttatcaatggc |
| | | gaaggacgcctggaagggatttacaataaacagggcgtcaaaacgtcatggagtatggatcgctaccatgctgcggagcaatttaa |
| | | aagcgggcaggtacgctttattgtttgtactgaagccggtggtgaaggtattgatttgcaggacaactgttattccatgattcatg |
| | | ttgatctgccgtggaatccgatgcgtcttcaccagcgtgtagggcgactcaaccgctatggtcaaaaaaatcaggttgaagttatt |
| | | actttacgcaaccccgatactgtagagtccagaatatgggacttgttaaacagcaaaataaccacagtcatgcgttctttgggcga |
| | | cgcgatggaggaaccggaagatctgttgcagcttattcttgggatgagtgataaagtttttttcaattcactttttgctgatggcc |
| | | tgacacaaaagccagaaactctaaatacgtggttcgattctagagcagggaccttcggtggtcagtcagccgtcagcgtggttaaa |
| | | ggtcttgtaggccatgcggataagttcgagtatcagaacttagatgaggttccgaagcttgatcttatccatatgtatggtttcct |
| | | cgagaacatgctgaaattgaatggacaccgtctggacaatgataagggtgttcttagctttgtcactcccaaagactggatcacac |
| | | agtttggtatcaagaagaaatataacaatatgacttttgaacgtgttcctacagagaaatcgttagaagtgcttgggatagggcat |
| | | gtgattattaataatgctattaatcaggctgagaaatttaacgcctctacggcagtagcaaggggtatttcctcagctttactgat |
| | | ttacacattgagagaccagattactggcgatagtaatgtacaatcattttcagttgttggagtggtactggaagataatattcaaa |
| | | ttttggtcaacgctgagttagtcaataaactggcttttatatatgacaacctacctaaaggttcgacggtgattaagcttgacagt |
| | | gcattccatgttaattttgagagggatataaagcgtgctgaggccgcattagatctctttattcctgggttgaatttaccctatga |
| | | gcaagtagtatggcaacatacagcaacttttttgccacagtaaatatagcagtgttcaggatagcattgggaatgagaaaaactat |
| | | atgaaaatatggtgctgataaagtattagtactatggtcgaacggctatgcgcttatgtcatggagctgattccagagagccttga |
| | | aaacgaaagatttaattttccccccagcgtcatccgctctggcaggtgagtcgcccgagtccgagtgcccagcattttcaaatcac |
| | | cat (SEQ ID NO: 38) |
| 42 | pLG044 | tgagaacttacacaattaacgccaattttcttattccatcacgcatacgataaccgtgatcaactttttctttttgcagcacccta |
| | | taatgcaaccagtttaatttctttggatgcgtaatagtcagtgtgctgctcttgataaacagtagtcaataggcatagtccatatc |
| | | cgaaatctaacttttattaacgtacaaatagcaaaagaataaataacttagagcataggtcctcgaaaaatttttctaatgttcga |
| | | tagtcttgcttttggcgtaatgtggtaagtccaataggtgataatgtgtatagttgcattgacctagtcttgtgagattgcattta |
| | | ggatctccatcatcaattcatctttcgattcaatttcaaaaaaggttctaaaatggcgggtgcttcaatagacgctattggtgtga |
| | | ttaaccaaatcaaagacaacttaacagaccgatacgaggatggctttcctgtccttaaagagatcattcaaaatgctgacgatgcg |
| | | ggtgcgaacgaattaactattggttggagtaaaggtttctgcaatgcagaaaatgaactactcaatgcgccagcgctgttttttat |
| | | caatgatgcaccactggcagaggaacaccgtgatgccattttatcgatagcgcagagctcgaaagctacatctaaggcatcagttg |
| | | gaaagtttggtttgggaatgaaaagtttgtttcatatgggtgaggcattcttctttatgtccgatcaatggcgaattgagcattgg |
| | | gcgtcagatgttttcaatccatgggataagtatcgtgatgcatggaatgaattcggtgaaaatgacaaatgccagatcgcaacaaa |
| | | gttaaaagggtttttaagtaccgataagccttggtttgttgtttgggtcccgttgcgtacaaaagcgctagctaaagcacacaata |
| | | actacattatcatcaacaactttagtggtgatgaaaaactccctagtttctttaatcaggctcacttatcagagaaaacttctgag |
| | | attttgcctcaactcaagaatctcaaagacatcggctttttctgcgagtctgacaagggtgtgtttgatgaagtgacctccataca |
| | | gttacatgaagattcgtctcgaagctctttttgcggtgaaccgcgattaaataatggagactcttttgcagtcttctcagggaaaa |
| | | tctattcaaattcgaatgaagagcgttgtgcactggactatgcaggatgcgagcgagtcatctttgatgagcgtttaaatcaatta |
| | | aaagacgaaaatatggggtggcctaagagttatcagttcgacaagaaagcgaacttgcctgttgaggctctcgacaaagctgaaca |
| | | gcatgcttctgtaacattttcgcgttttaaaacaaaggggcaagcgtacctcaaagccaactgggctgttttccttcccttaagcc |
| | | aaaccaaggaacttgttgctgtgcctatcgagggggagtacgactacaatctctatttacacggctacttctttgttgatgctggg |
| | | cgtaaggggttgcatggccacgacaatcttgggttttctacctccctagagcatgtaaaaaatgatgagaaaaagctgcgtgaggt |
| | | ttggaacatcattctagccagtgaggggacattcaacctcgttttaccggctctaaatgagttttgtcagaagttaaggctgccac |
| | | atcaaataaaaactgttttgaccaaggctttgtacgatctcctcatagaaagatatagaaaagaagtatccaagagcgccaattgg |
| | | ataatcaatatcgatgacaagggggctgcttggtctttacttgataagaatgcccaatgcttaccgatccctcgtccagagaatag |
| | | tgattactctcgaatttggtcaacgttgcctggtttgagtaagttactggataaaaagtcactgtatgaagccacgggtaatgaat |
| | | ttttaaccgagcagaatcaacgtgatagttggaatattacgctcctggaagaagcgttaggaagtggtgttgtcaacgcattttac |
| | | agatcaatcaatattgaatatctgcttcagttccttcaactagctaaggagcagtgcacgacggaagattttgataacctgattat |
| | | tccacagttccgagaggtattgtctactcataagcttgctgaactttcattgaacaaggctcttaacacgcaagtttttgagcttg |
| | | ttagcgcacctaaaaccgtcgtactaccaattgataaagatgatcaatctatttgggaacttgtctgcaagatcattcctgcaaag |
| | | ctactgctccctaaatttctgtctactcacaataagccaattcatgacaatgtcactgaagaagagctcttcgcacttttaaccct |
| | | agtagatagctacatcaaaaaacagggtgaacgtttatcctctgatgaatcgtctgcctgtgagcgtctcattacatttgttattg |
| | | attgtgtaaatgcaagtgaggtaatccaaaaaagcgatttttatcagaagagtgggcatttaaagcttctaaaagtggaagctctt |
| | | ggttcgcaacagagcacaaaatatcgctccttaaacgaactcatagtgttaaaagaaaaataccagctgtttcttcgtggagggga |
| | | gcggaactttggtaaagggttggggaaagagctagttgcagtcgtgcctggcttggagctttgttttataagcaaggattttgaaa |
| | | ttggtggcctatatgaagggcttaccgcttgttctgaagccgcgtgcctacgactgctttccacgtacccaaatcttggttcaaat |
| | | tcggcaagactagcgctcactaaagtattctctgccgagctctctacagatgaggagaaaagaggtttccggtatttgattcacgg |
| | | cagcaaagaagacgacttgagacaaacgctttggaagccaaacagggcaactaacccagtatggatgaaaatttggcgtatgtgtc |
| | | agccagaagatttccctggatggtgtgagttagatgaagagttttctaatgctttgacaaaccagtacgaacattttattggcgtt |
| | | aaagagcagttctataaagacattatctctgaatacagaacaatactgcctgaatgcaattttgataactttgatgactgggaagt |
| | | ggagcaactgctcgcagatattggtagtcaaggagatgaaaggctatggaaagcgttgcctgtccataggacagctcataacacta |
| | | gagtcgcgattacgaccaaatgcctgatggaaggaagtgcaacagttccaagtgaatgggatgttcaccttattcaacattcagcc |
| | | attgctgaagtcgccgcttgccagcataaatgggtgaatcatggtctacctaaagagctgatcgagattgcgcttacccaatcaag |
| | | tccagctcagtattccgcatttattttggaccagctctgcgctattcgtattgcgaatgaaggaattgagcatgagttggaaggca |
| | | agataaataataccaagtggctgcgattagcgtcaggaaccgaggtttcaccggaagctattttatctttctctgccaatgagctg |
| | | cctgagtctgcaaagttctgcgagttaaaagagtcaaacatttacatgttctctcaactcgatggaaacatgtttgagcacgatca |
| | | agcacgtggtttcttgagagagtgggtcgcaaaaagtaacagctcagtttgctcgtgcattttggcagaagccgcgcaacatcaaa |
| | | gttatgtagttggtaatttttccaacatttctgctcaggtgctagaacagatttcatgcatcccgccattgatgcagctatctgca |
| | | ggctggggcttactggttgagctctaccaaagccaatatctttcagtgaatgaaaacaagcaagtgatgctatgtaaggaaacaga |
| | | accacaatcattatggtgggcgctggagcgtattgctgatgatgatattcacggtcagtcaaaggaacttcggaaagcatttttag |
| | | aagcgttgtgtaacaccgagggaggcgttgattatcttcctaaactgagatttcgcaatgagaacggaagttatgtatcgggcaac |
| | | acactggtatcgaatgttgctcaggtagttgctgataacttaatttcgccacaagaatacgcagtcattgagagttattgcagtaa |
| | | atctgctctcacgaatggtaatacgtcaaaaatcattgagttagcgggcgataatgcgccagtacttagtgattacttcgatgact |
| | | gggaagggatggttccccctgatgccatagcgacatttatagcactgtttgctaaatctggtggcgtcgagaaattggttaacaat |
| | | tatctaagacagtcaacgctggagtcgataaagcaggggtatgaggaaaagtggaactccggaaagggacgtagaggcgaattttc |
| | | acactatccgtatagctcgttatataaaagtgttgattttgaactggcaatttgtgcagaaaatgcggcgtacatgacgtcgattt |
| | | tcggcgaaagaattcaagttaaattacaaaaaacaccagattcattgcttgttcaccaagcgaacaagtccaagacgaaaaggata |
| | | gagcttcgccgagttgatacaaagaatgtatcaaaagaccaacttctccgcatgcttgccaaagctgtagaaacgatttttactga |
| | | tgtgtttggtgcagagtgtattcgatttgaaagtgaatttttgaagaggtttggtgcttcagaacaggtagatattcagattaccc |
| | | gacagatagtcttggagaatgttgtccccctacttgaaaggcttcaagtgcgagaagaaggactttgtgatttacgttcagattac |
| | | aaacgtgaacagcgtgttttggcgagcagtgatccttctgtactacaagatcgctcacgccttaacagcgtccttacgaagattaa |
| | | agagactcttgaaaataacgaaaaagtgcaatctttggtactcgaatctgtacgaaaagagatgagtaaacatttccaatactcgc |
| | | ctttcagcgtgccatttgagctgtttcaaaatgccgatgatgctttgtgtgaacttattgaaatgcagggcgactcaaccaatgta |
| | | ctgactcgatttgatgtggtttctggcagtgatgggactcttaacttctaccattgggggagagaggttaactactgtaaaagttc |
| | | atatgtcgcaggcaaaaaccaatttgaccgcgacttagaaaagatggtgagtctcaacgtttcggataagtcagatggaaaaacag |
| | | gcaagtttggactgggctttaaaagttcattgcttcttaccgacattccacgtttggtgagtggtgatatttgtgcagaaattcat |
| | | gctggcgtattaccgagtgttcctagcaaaccagtgatgacggaacttaatcaaaatgtcgatgagtataaaattggaaatcgtaa |
| | | accgacattaatccagttgcctaaatgtgataagaagcgggcagatttgaagttggttttgggacgtttcaaaagtaacgctggca |
| | | ttctcacggttttttcacgacaaattcgagaaatcaatattgatgagcagcgatttgggtggtcgggacaggctctccataatatc |
| | | cctgaagtacttgtcggtgaagtgaaactgccaacaaatacttctgaagagtctaacgttatccttcgaagtaatagagtgcttat |
| | | tatcaataccgagtccggtcagttcctttttgctttggattctaacggagttgtttctctttcgaatcgaaaaaacctaagtagct |
| | | tttgggtgttaaacccgattgacgaagatctgaaattgggtttctgcatcaacgcgccatttgcggttgatattggtcgctctcag |
| | | cttgctgtagataacggagacaatatcgatctttccagttcactcggcaaagcgttatcagctgtgttggtcaaaatgtttgcagc |
| | | ttcttcgaataattggaatgaatttgctgaagaggttggcctgggacaaagcagcacatttatcaagttttgggcgtcactttggg |
| | | atgtaataacagcccattggccagcaaggcttggagagacgaactctaaagctgaactgattaaacaaatgttcacagtggaagat |
| | | ggtctgcttgcgttttaccagagatgtgcggctcttcctcgaaatcttggtgtaaaggaagattctcttgttcaacttaaaaacgt |
| | | tgatactggagcgaataaacctttgaccaaggcatttaataccttgggaaatcacccgatacttcaacggctatataaagaccaac |
| | | aactcgtcgggcatgacacctttgagtttttgaagagtatcgattttagaccgaataatggtgcgttaactaagctcgaattgatc |
| | | gatttgattggacaggactttcctcacaatgaagtaaaccacgacagagcaagtttctatggtcgcctatttggtaaaaactttga |
| | | aaagttaatgtcgaattttgaaatgacagtgactgagaaaaaggtgttggaagagcgtttttctgaattgaagtttctcaacaaaa |
| | | ccggtgtatacgtgactgcaagcaaactgattgttgaggggagccctgagagagacttgctatccaagtttgcaccagacagcgcg |
| | | aagttaagtgaaaaatatgaccaagcatcaatggacttggttagcttcattcgtcgtgacgtaagctatgacattcattcatgggc |
| | | taagcaaataagatctgaagaatctaacaggggaggaaagcaggaagggttgtgtagcttccttgttgaaggcggctatttagcat |
| | | catcgcttctcagaaaactacagacggatcaccccgcgtttcttacaaagggacgttttgatccgagcgtattaacagaaaaatgg |
| | | cgttggagttcttcaaaggcttcggctttcattagcatttggattgatacagaggaagataaagcaaggcacgtacgacaagcgca |
| | | aaaagagtttattccgaatgtgaccaatggtgagcagatcctcgaaaacatcacgaactggtggaatcaatgtcgtaatcaaagct |
| | | taattgattatgacaaacagctctatgctcaaccaatgccttggaaggcaatgacagaggacttcgagcttgaaacgttagaggtt |
| | | cgtaaaggttggttgaagttgttctatttagggagttgccaaacattaggtttcaataacgatgtagctaatcggaatgttgtttc |
| | | ttggttcgaggacaaggggtggtgggataaactagccgttgccaatggtcctagccctgaagtatggaaagaattaatggaagaat |
| | | atcttcaaacagcacgcgttgatgagcgttatagagtttggattcaagttcttcctttgtatcgctttgctactaagctcaaggac |
| | | tatgtcgctctcttcatgaacgcttcctttattgataatcttgatgatttgttaaaaccaaatagttcaaacaagttatcaggctc |
| | | tggcatccaagtatctgagttaaaaggaacgctcggtattgggattaatttcattttacgagagttgcaaaggcaccaagttttgg |
| | | agcgtgagtattgtgaagatatccaaaagtacgcatttgttttgcctgctcgattacgaaagttactcaaaaaaatgggagcaggt |
| | | ttaagctttgacgcagagccagagaattcagagcgagcttacgactatttcgtttcggcattaaatagtgaaacccaccctcttct |
| | | taaggactttgacatcccatttagagtcttgttggctgataagcaagcgtttgaacgttgttttaattttgctctagatgagcagt |
| | | ttgaggaagtatatggataacattatacgcgttattcacccaaaattcggtgtcggtaccgtcgaattcgaaaaagctgagacatc |
| | | tcttgtccgatttgaacatggttttgaggagtgtttgaaaagtgagcttgaggcggtcgctgatcttaagtccgatcttgtttctg |
| | | gacagagtgtcgctgcctctgaacttgcgttaaaaacattagcgcactcactaaaaagtgttaatgaaaattggagtgttttttct |
| | | aaatcgaacattaatttacttcctcatcagttatgggtatgccatcgagttctaaggcaatggccaacaaatcaactgattgctga |
| | | tgatgttggtttaggtaaaacgatagaggcgggcttgattttatggccccttatcgagaggaaaagagtcaagcgtcttctgattt |
| | | tgacgccagcacctttggttgagcagtggcaccaaagaatgcttgatatgtttgatattcgtttgagtatgtatgcaccagaaaat |
| | | gatacctcgcgcgtcaattactgggactcaaacaatatggttgtcgcttctctacctacgctaaggaacgacaagaatgggcgttt |
| | | agagcggatgttaaatgctgagccgtgggatatgctcattgttgatgaggcgcaccatctaaattcaacggaagataagggtggaa |
| | | cgttaggctttcgctttatacagacgttgattgaaaatgataagtttgaatcgaagttattttttacagcgacgccgcatcgagga |
| | | aaagaacacggattcttctccttattgcagttgctgagaccggatttgttcaacgttaagcaaatggatgagcgagaaatgcgccc |
| | | atttgtgaaagatgtgttgattcgaaacaataaacaatttgttacggatatgaatggtgagaggttatttaaacctctgtctgtgt |
| | | cctcaagaacttacagttacagtgaacaagagcaacatttctatgacctcttaaccaagtttattgtatcgggtcaagcgtatgca |
| | | tcctctttgaattcaagggatcaaagagcggttatgttggttcttaccgcaatgcagaagctcgcttctagttcaattgcagctat |
| | | cgagagagctctaaaaggacggatagagaaacataaactaggtaagcaacgtcttcaggatattgaagttcaacaggctgctttat |
| | | tagaaaagcgtgaggagtcagaatcgcagtctgaaagcgagatatacagtgatgaattagcgcaattagaactggaatttattgaa |
| | | acgacaacgcgggttcaattgatggatgatgagctccctagaattatggagttgttgtctgcttgtcagaaagttggctctgaaac |
| | | aagaattttaacaatattagatatcctagaaacggagttcaaagatagaactgtcgtcttttttactgagtataaagctacgcaag |
| | | cgctattaatgggtgctttgaataaaaagtatggtgaaggctgcgttacttttattaatggtgaaaatcgtcttctgaatgtagag |
| | | aatggctcaggagtatgtgttgattatgtcaccgatagatacaatgccgcgaagcgttttaatgaaggcaaagtacgatttataat |
| | | ttctacagaggctggtggtgaagggattgatttacaacaaaattgtttttcaatgattcatgtcgacttgccttggaacccgatgc |
| | | gacttcatcaacgtgtggggaggttgaatcgatatgggcaagtcaaaaacgtagaagtaatcactcttcgaaatcctgataccgtc |
| | | gagtcaagaatctgggatttgctgaatacgaagatcgatttaatcatgcgttcggttggcggtgcgatggatgagccagaaaacct |
| | | aatggagttgatattaggtatggcggatagcacattgtttaatgagttgtttacagaagcagccaatcgtaaaaactctgaatctc |
| | | tctctgcttggtttgaccataaaacaaaaacattcggtggcgagtctgtagtgcaaaaagtgaaagacttgattggtagagcagaa |
| | | aaatttgactatcaagatcttgaggctgtaccgcgtttagatcttggagatttaaaaccgttttttactcagatgctttcatttaa |
| | | tcaaagacgttgtaagtatgatgaaaatggtggtttatcgtttttgacacctcacgcatggttggggcaatttggaaccagacgct |
| | | cgtatgagaaattgcattttgaccgcaaagctaaacagcttgattcagaagctgacatcataggctttgggcatcccatgttttca |
| | | aaagcggttaatcaaggagagcaaatccctggaagttacgcgtttcttaacggtatagagaaagatcttgtagtgtttaaggttca |
| | | agatcaggttacgggaaccgatgcatcagtaaaagtgagtattgttggactggtgctcgatgataatggcgattgtgaattggtca |
| | | aggacgaagaccttatcgggtatttaaacgagtatcttaaaatttccaatgatgttgactctaaacgtacaccagaggatttagtg |
| | | tctgttattcaaactgctaatgattatctaatggagaatgtgtcatcaattggcttaccatttaggctgcctaattctgaaccatt |
| | | aacggtattctacaaagcaagtaactaactattattctatagctgagcattacgaaaaagttcggtagtgattctggcttaatatt |
| | | tgggccgaagctaagaggtcgtt (SEQ ID NO: 39) |
| 43 | pLG045 | gtcatagtcccttacggagataattcattgaaattaatatcttatacagcacatgtaaatagccgtggtgtatttttatccaatga |
| | | atcgttacaaaaataagatgcatgcccaccctgttctgtgtgaacgctacgaccagctacggatttataccaaaagtaggaattct |
| | | atatgtcacgtattaccatcaacgttttatggttaaccgtaccaatagcgcggaagtgggcatgagcgaagtagcagatcaacagc |
| | | aattggaaactcagccagcgggtgatgacctcctgcaaggtgtcaaacgcgttctcaggcatgccgttcaggcgtacggggatggg |
| | | ttaaaggtttatcaaagcctgcaaaatctcaacgaggtgattggcacggagtacggtaatcgggtcatttatgagttgattcaaaa |
| | | tgcgcatgatgcgcatacgtccgaagaacgtgggcggatagctgtcagcctggtgcttgaaaacctttcacggggaacgctctaca |
| | | tcgctaatgatgggcgagggtttcgccatcaggatgttgaagcggtcaaaaacctggcgatcagctccaaagagattggcgaaggt |
| | | attggcaataaggggcttggatttcgcagtatcgaggcgctgacgcaatccgtgaggatctattctcgctcaaatacgaacggcaa |
| | | ggaccgatttgagggttactgtttccgtttcgcagatactgacgaaatcgcgcataatattcgcgatctcggtgttgatgacgcga |
| | | tcagcaacgaagttgccaaaacgcttccccgctatcttgtgcctgttcctctagatgatcaaccggaggatgtccgcacttttgcc |
| | | cgcaacggtttctccaccgttatcgtggcaccgttagaaactgaagcggcagttacgcttgccagaacgcaggtgaaggagctgac |
| | | caatcgcgatgttccactgatgcttttcctcgatcgtattaccgaaatcagtatcgaaattttatccccggatgagaaagccgaaa |
| | | agcgcaccatgcaacggcaggaaaaggcgctgggaagtattcctgacgcgcctgatgtcagtctctacgaagtcgatataggtcag |
| | | cggaaacgctttttagtggccagaagcaatgtcgataaagcgcgcgtgcagcaagcggtgagcgatagcttattgactgcacctca |
| | | gctaaagcgttggctgaactggcaagggataccggttgtttctgtcgccgttggcctgaacaaatcaacagtaacttctggaagac |
| | | tctacaactttttgccaatgggcactgaggccgcttcaccgatttgcggctatatcgatgcaccattttttaccgatattgacagg |
| | | cgtaacacgaacatgagtttgcagctgaaccggctgttaatggaagtggctgcggaaacctgtgccgctgctgctttgtccgtcgt |
| | | atcccgtgagctggatataggtgcatctgcggtttttgatctgtttgcctggacgggggaacatcgtcgcatgatgcaaacagcac |
| | | tggaacggaaagatacttcgctcagcaaagcccgcctgattccggtgatggctccgccaggaaaacagcaatggtcgagtcttgaa |
| | | gaagtcagtatctggccggaggtgaaatttgccatcctgaagccgaaagacgttgccagatacagtggcgcgcagttggtttctag |
| | | cgaattgaatacgccgcgcatagtgcgtttgagggagataacaaaatttccctatatgtatcagtcattagatccttcggcgcaga |
| | | cactggtgaaatgggcagaagcctttgccctttcgctggtggaacggaaattctcccctgccagttggaccaaattctatgatgat |
| | | ttggtcaccttgtttgctgcggtaaaagtgaaactcaacacacttgagaactgcctgatcctgtatgaccgccagggcaaactccg |
| | | gcccgcaggcgggcataacagtaatgaacacaatggcgtttttgtacgtcggcatgtatccagaggcgacaaaaagaaagataagc |
| | | gtaccgggattccgttgccgccagcgattgtttctcggcgctaccggtttctggatgaaaaaatcgtgcttagtgcggcgacgttc |
| | | aatgcgtttaccgtcgccgacctgataagagagtacgatccgatcaaagccctgtcagggctgaatacggccctgagtaataaggc |
| | | gacagtcagacagcgccaggatgcactattgtgggcatttgaggtctggcgcagcagtagtgtcgttgtcgatgtggagctgaaaa |
| | | aagccgatctccatattcccgtgcagtcgggttggtgtgcggcaagcaaggctatgttttcatcctcctggacgccaacagggaag |
| | | gttgtggaaagctatttaaccggcgcgatggggatctcgcctgactgccgtctggcagcgggtttgttattgattgagctgcaaga |
| | | ctggccgggcgtcgtgcaaaacagcaaaaccgactggattaaattcctccgcgtgcttggcgttgcagatggattacagccggttg |
| | | aatctaaggtaagagcgcgagcatatggcgatagttggaatagctttttacgcaatggcgacgagcatgaggggtttgatagcgac |
| | | tggagggcagaagtaaagcgggcacatataagtttctaccatcctcagacggtctatacctcggaaggaaaaacatggcgattgcc |
| | | cgggcaacttgagcacgcaacattgccagacgatctgagggagctgttgtgtacgctgattttcgcctttctgaagtcgcagacta |
| | | cggagttttttacctttgaggtcggtcgttttgagcgacagaattcgcaaacagactcccgtacgctgccaacgccgcttggcact |
| | | tttttacgcactaaagcttggcttgccagcactagctcactatctgaaggattgcattttagccgtccagatgcgtgctgggcttc |
| | | gcgggagcggcgcaataaacctccgcgtttcctagaccatttgattgagcacaacgttgatattattgaagagagtcaactagcgg |
| | | agcgcttgttttctgcgaaaattggcctacgtgattggaatcataccgggacggcgttggatcgcattaaagaactggtctacatt |
| | | gttccgcagttgaacgctggcgataaggcggatttacagcgggaatatcaacgaagctggcgtgatatcctcgacagcgacgaagc |
| | | tcttcccgacggattggacctgattgtttttcgccgtgggcagcatgaagtgctgcgcggcaacagcgatctgcctcctgcggtga |
| | | ttgtcaccagtattgcacaaaaaattgaagcacaaatgcttgcttctgcaggctacgcaatactcggtattggcctggatgagacc |
| | | gatacactcgtctcctgcctcggtgatacgggacgattttcaccccgtaagattaatgacggcggagtgcaactttacctcgatgg |
| | | taagccgttttatcccgatgagagcgatccgttgcttatctccttcgacatgaactggttaccggaaatcctggttattggtctgg |
| | | cgttactcggggaaaacttagagcggggcgttcacgccaccaaggttgataagcagctgcgcgcaatcagggtacgccgttgtaag |
| | | accctctcttttgccgtgcagggcgatgatgccaccccaacggagtcgttcgtcagctattcctggccccatgaaacgatgccgac |
| | | gctgattattgaagaggggctggtgtttaactggcagaccttagcgaagatttcccgcaacctctcacggctggtggataaccggt |
| | | tacgtttcattgaaaccttacttttgcgcctcgcagttggtcgcgataatggctcgttgagtaaaccggatgacgttaccctggct |
| | | tgggagatgaattgcgatgttcaaacgatccgtgatcattacgcccgactgcgcacggacatcactcatgtgatagacatgctact |
| | | tcctgtggtgacgtatctcaacggtattgagcttgctcaggttctcaagcgggaatatgccttatctaggtcagtatttgatgtgc |
| | | gtagttggatttcatcacatctatctgatagtgatatacctgctgaaaagctgctggacgtgtgtgaaacagcaaccgatcgggtt |
| | | gaactccgtaaaatgctgtcgtttgattttcagcaatttaacctggctctggaagcgttaggggaaacaccgctgtccaatgagga |
| | | tgctctgcgcagattttttacggcctttgtcgggcagaggcgttcacatattatcgatcggttacgccgacactatctggcgacct |
| | | ttgataccggcggagatttgtcacaatacgttcagcataaatctttgggcttcatttccttcaactctgaatggattttgacacat |
| | | gaaaccttggaaaaggagatggtggactcgcaggttgacacgcaacttttgagtgcgttaggaccggacaatggtgaagagctgtc |
| | | tgcacttaatacgttattagacgcgaatcgtaaaaatgtgcgcgaatttgccatgcaggctcagccgcgagtttccgcctggtgca |
| | | gacaaaatgatgtcccggtgaatgctcactggcagtacaacgatcctcaggcgttttgccgacagctcgaaaataagggctttctt |
| | | gatttccggctctttgagccggattcactaccggattactgcctgcgcgccgggctatggccaccaacgatgccgcccagcctaga |
| | | tcaggatgtgctgaatatcgacatgaggaaagtttcccaggaaaaagaacgcgctgagcaggcaaaacggcaacaggaacttgagc |
| | | gtcgcagtatctttttttccgggcagtcgcttgatacagccagcccgctatttgccgatcaacttcgggaactggcgagtaccgat |
| | | agtagttggcaggtgcgcagccagcacaagacgcaggccttgatggattttggcgtggtgacaatgcgtcaggcgagcggcggagg |
| | | ttgcggaaaaagaaccgggcgtgcgtatcgggagcctcgattgacacctgcacagcagcaagccatggggctggcgagcgagtggc |
| | | tggcttttcagtatctgcgcgatcgctttccggattatacggatgaaacttgctgggtatctggtaatcgggcttcgttttgcggg |
| | | ggcgaggaaggagatgattcggccgggtatgatttcatagtgaagacgccgaaagtggaatggcttttcgaagtcaaatccaccct |
| | | cgaagatggtcaggagtttgaactgactgccaatgaacttcgtgtggcaagtgcggcggctaaagacgcaagccgacgttaccgaa |
| | | tcctctacgtcccttatgtgctttcgccggatagatggtgcgttatcgaattaccaaacccgatgggcgataaaacacgcaatcac |
| | | ttcagcgttgtggggcatggatctttgcgtttgcgttttcagcggcaggagaactgacagcaaccctgctcagggaaacctgagcg |
| | | gggtttttaaatatggcctctatggataggggacactttctgcagtaaatggataataagaaagctaacgttgaagtctgattctg |
| | | ccattttccacgacagctaaatgctggatcttctttttaggatcccaacatacctagcagtaggacgtaagtatgcttgagttcat |
| | | ctcgatatccttgtttctgaatgacaggcattactatttcgtgggtgtgaaccgatgaagggggtgatgtcattggaaaataatga |
| | | ggtagtagcaaggagaagttctgctcttatcatagtgaaaaagcggtttgggaacaaatcggaactgata (SEQ ID NO: 40) |
| 44 | pLG046 | cactcaataccacacaattctcaactccgaaggacttcgtgaaacgtgagtaagcgtcaactcagctccgtctggtttacctcgtc |
| | | aggctctgtagtttaggtgttgccatggcgtataaccctgccaacagaataacttaccttactccagtcaataccgccttcgctgt |
| | | acgcttacgcttttcgctcaaactgtgtgaaaacgtttttgatcgcataaattaccaaaacagggctgaaaaccgcgctcatacgt |
| | | aaaattcggctcaactaaccagtcgaccaatttcagattttgcgtagacgcgcgcacttcagttttagtcagggttttcacacagc |
| | | ctgcgctcatggctgctttaagctaaaacaaacagatagaaagaagttacgataccctgtgaattcttgcaggcagatatcaagga |
| | | gggttcattggtagcgataaaaatgtatccggcaaaggatggggatgcttttcttattatttgcgatgaggaaaaaagtgcatttc |
| | | tgattgacggaggctacgcggaaacgttcaggcaacatattttgcctgacttacgtgagctgagttttaacggttaccggttacgt |
| | | ctggtcatggcaacacatattgattcagatcacattggtggtctcgtggacttctttcttgtaaatggacacgcagcagagcctgc |
| | | agtgattactgttgaccgcgtatggcacaacagcctcagggcgatgacgagacccgaaaataatgcacaaaaagtggattcccgag |
| | | aaatcactgactttttgagacggagatatcatgtcgaagccgataaagccaaaccgcatgaaatcagcgcgcgtcaggggagttca |
| | | ctggctgccagccttctggctggcgattatcattggaatgagggaaaagggtatcagtgtatctgcaccggtacctccattcccaa |
| | | cttgatgtgcgataacagtctaacaattctgagcccctctaaggagagaatttcagcgctctgcctgtggtggcgcagacaacttg |
| | | catcgctgggcttttcgggacggtcctcctcgagtgaggcatttgatgatgctttcgaatttttttgtaaaagggaagcatctcag |
| | | gttcctcttccgcatgtcatcaatgcaagaacaccgttgcttgagagggattatgcacgggatacctcgccaacaaatggcagttc |
| | | gatagcgttcagtctggtgctcaataagaagagaatattgatgctaggagatgcctgggcggaagaagttgtgacatctctgggtg |
| | | ccagtggggcgtcccatcattttgatatcattaaaatctcacatcacggtagtattagaaacacaagcccgaatcttttaaagatc |
| | | atagatgctcctgtgtacctgatctcaaccgacggaaaaaagcatgccagacaccctaacctggcggttctgaaagcgattgtgga |
| | | cagacctgcggcgtttacgcgaacgctctattttaactatgccaacagcgcatctgcttttatgaaaaattacctttctgcaagtg |
| | | gtgcacaattcagaatcattgaaggatcaacggattggataacactgtgagatatgctgctactgaaactgaaataaggaacgcaa |
| | | ctgtactcattgaatgcgcgggttacactggttccggaaccctgatcgcagcagacaaggtccttacggctgcacattgtgtagta |
| | | tcggatgatcctgagacaccaattacagtgacattttttggtgcggatgaagacgtctgtgtcaatgcgacaatttcagaaataga |
| | | tacatcgtgcgatgcctgtctgctaacactttctgactctgtcgacattccgcctattacacttatgacacagccggagcgagagg |
| | | gaagccaatggaaagcctttggctatccggcatcacgcaatgggccatcacattatcttcatggcactataagtcagattttacca |
| | | aggcttttccatggcgttgatatggatttgtcggtcagtgccgattgtgttctggaagagtacagtggagtttctggtgccgccat |
| | | tctatcagaaaataaatgcattgcgatggtgcgcatcaggatggatggtggactaggtgcagtaagtcttgataagttaagcggtt |
| | | tgctgattcgaaacggcctcatcccagatgacattgcatccctgccagattcatcactgtcgggtgaagttgtcctgaaccgcaca |
| | | gaatttcgcgacaactttgaatcgttcgtcctggagcacaagggacgtgcagtgcttttggaaggtagtcccggctctggtaagac |
| | | taccttctgccgccattatcagccccgtagtgagcaactcgcagtggcgggtgtctatgaatttacaccggaagacggtgctggta |
| | | cgacattcaaaattcttcctgaggtatttgccgattggctgcataaccaggtttctatactgctttcaggtaggcctgctcgcagg |
| | | gaggaaacagaaaagatcaatctgacccaaaaggtgtctgaccttctacatactttctcagattactggaagcacaaaggaaaata |
| | | tggcgtcattttcattgatgctgtgaatgaggcaagcgagtgcggggatgaggcagtatcgcgctttacagcattactgccggtga |
| | | cacttccggagaacgtcaaacttgttttcaccgcaccatcattatcatcagctggtaaggctttccggcactggctcacacctcag |
| | | gattgtatcagcctaacgcttttaagccatagggaggtgttacagctaacagctcgagagcttaaaacttccgccccttctttgtc |
| | | actactcacacgagttagtgatatagctcagggccatccactttatctccgatacattcttgggtatctgaaagcgaatccggatc |
| | | aggttaatctggagatattcccggttttcagtggcagcattgaaacctactacgaaaggctctggcaggggctggttaaggatgag |
| | | agcgctgtaaatctgctcggtattctctcgcggatgcgctggggcattgatatttcatcactgatccctgttctaacaccgcagga |
| | | acagacggtgtttgttccaacccttgaccgtattcagcatctgcttcttaatgataaatcatcagcattgtgccaccaatcatttg |
| | | cggcgtttatcaacagtaaaacggcggtaattaactcgctgctgcacggacgccttgccgacttctgccttaccagtggagagagt |
| | | tatggcctgattaatcgcgcttatcacctgctcctagcctctcacgacagacatcctgaagccgcattggtgtgcacgcaggaatg |
| | | ggctgacgcctgtatcgtcaagggggctcagccggatattctaattcacgatatccgtcagaccctgaagaacacgcttattcgtg |
| | | ccgatgcagtggcatcgattcgtctgttgctgcttttccaacgcatgaccttcagacaccattttttgtttctgcagtcagcttat |
| | | cactcaggccttgccctggctgcacttggcagaccggatgaggcccttgagcagctcataccatctggaagcctcgttgttgatgc |
| | | agttgatgcaattgtcagcgcacagactctcgcgcgtatgggaaacagtgaacacgcgctgaagctattggaaaaggtgaagtcag |
| | | ctgtcgaccaagaatttgaacgcaatcccgtcaatctatctgattttatcggcctttccctggcttgggtgagagctgagctgatg |
| | | gctggggtggttgatggccacggacgcacacgcgaggttgttgagtatttgtacggttgtgggcaagtcgttcgcgataattttga |
| | | acaatcagcgcatagtaaatcagcatatacacgcgctttttatcctcttcaggcagaaatggaagccgtgaacatagcctttaatg |
| | | accgctccgtatctttacggacggttaaagaaaagtttggtagcttaccggaaaatattcttgatctgatgctcagttcagttatg |
| | | cgggcacatgacatcattctgcaacatcagttgccgatgccccagcatgctttgcaacccgtttggtacaatctggacagattact |
| | | tcatactgatattccgtattcgaacgaaattcgttttaattcattaagtagccttatttttttcaatgcgccttctgctcttatta |
| | | tcaggatggcgggggtattttctttcgaagtagtacccgaaataacgttgctcaatgaagaaaatgagatagcagcagacagcatt |
| | | gacgttagtgaacagggacaactctggctggtgagcgcctaccttaatgaaacgcaaccctgtcccgatattaaacatccgagtca |
| | | gggatgttctgaatggctcaagacattgactgaggctattttttggtacagcgggcaggcgcgccgggcagttattgacggcaacg |
| | | atgagaaaaaagaactgcttttagtcaaggtgcagaatgatattctccctgctctttcgtactcgctggaagagcgcatggcatgg |
| | | ccgaattcatgggcaatgcctgaacagattatccccatgatttacgaagagttagtaaacatgttcggcgcatgctggcccgataa |
| | | gatatcagtgatcactgatttcattctggctcatacgcctcagcaatgtggactttattccgaggggtacaggcgtttactgaaca |
| | | gagttattcagactcttctaaatgagcatcggtttttggggcaatctgatacgacatttcaactacttgagacgttgcatgcgttt |
| | | gtttctgcttttactgagaatcggcaggagctggttcctgaattactgaatattattccagcttatattagccttgatgctcctca |
| | | gctggcacaggacacttacactgagcttttaggtgtgtcgatgggccctgactggtacaaagaagaccaatttgccctcatgacaa |
| | | ctatgctgcgcgtgataccacagcatacagacacaaatactacactttcacaagttgcaggattccttgaacatgcttcgggtgaa |
| | | atgacatttaggcgttatgttaggcaggaaaaatcacagtttattggcgaacttattcgtcgtgggaattatgcacacgggtttaa |
| | | ctattatcgtcagcagtcctgcggatcccatgaggaaatgctcacccaacttagccacccagctgcagatagccctcatccattga |
| | | aaggcatgcggttcccggggggagcgctggatgaggaacatgctgtagaatgcattgtcagtgaactgcgaaacagagtcgactgg |
| | | cggcttcgctggggacttcttgaaatattcagctttggcagtattggtaatcttgcagtgccctttgctgaacttatcaatgaatt |
| | | ttctgcagacactgaagaccttaatgaaatacccaaaaggttgcacaacattttacatggtgatgtgcctttctcagaacacagaa |
| | | attttatcaaaaatttcacagagcaccttgcagacaaccataagccactctttgctgaatttatcagtttgctatccgaagacact |
| | | agcgataacgacgttaagcctcccccctctggtgatgctaaccagaagggtactgatacctcagatgatgtggcaatgcagccagg |
| | | actttttgggaagcgttctgcgatcaatagggctgaagcctgcatggaaaatgcccgaaaagccgcagcacgcagaaacacagttc |
| | | gtgcaagtgagttagccgttgaaagcctgcatataattcaggatggtgactggtcagtctggagaaagaacaaccatctggcggaa |
| | | cttacacggacgtacatattggacaactctgcggatgcaggttcggtcattcgtgcttatgcttcgcttgtagaaaaagaacgtta |
| | | tgccccggcatgggtaattgctagtcatctcatcgaaatagcagccagtaaattctctgatcaagaagcccaagctattaaccaga |
| | | tcgtacttgaacacaaccgccacatgcttgggaataccgaagcggatgctgcgcatttttcttttcttaatgaacctgatacctca |
| | | gatgcaggtgaagaaacactctattttctgttttggctgctggaacacccactgaaattcagacgcgaacgggctctggaagtact |
| | | gaagtggcttgcatcagacgatgataagattctgggccaatgcgtgacggaggcactcgtttcagacattgcctcacgagctgaag |
| | | cactaatggcattgacagactgggtgtcagctagatctcctcagcgaatatgggactttatagttaaagagcgcagcctttttgaa |
| | | tggcttgaaggcactactgcactaagccaagtccatctcctggagcgagtaaccagcagagcgggatttgttttaagaaatgagat |
| | | tgccgcatttgagcgaccccgaaagcttttactgacatcagaagcctctggacaacggaatattccagaaaatttaccaacatggg |
| | | tgcaatccttgtcgcagacccttgccgtgatggaaaagcagggaatagatatcccagctttgcttaccttactcgaaaaacgggtt |
| | | ttacagcagagtggattggctgatatcacggtggcttttgagctggaaaagttacttgcgcgtggttttactgtgaatagaacacca |
| | | agtcaccatcgctgggagacgatggtgcgatttgcattaaaccagatcatacatgaggcggccgcacaggatgaactgcaaaacatt |
| | | gaacccttgctacgtgcctggaaccccgcgtcagaggagtgtgttgagccgtgggaggtttgtaaccgggcaaaacagattatctgc |
| | | gctgttatggaaggtagacatcagcaagcttcgggcatagaggatggctttttcttgcattatcttgatgaagtggaggtttcccga |
| | | gaaggtcaaacgcatctggtggaaatctcagcggtgttaacgacagctcataatggtcatgagagccttagaccaggtgcagaaagc |
| | | gaatttaatgcaacacagacacctgatatagagcggacgcttagtgtgcaccttacatgccagcgagtcaaaatgcagcctttgct |
| | | ttttgggggagctacgcctgccgcagtgtcgaaaaagtttatgcagatgactggaacgttgccttcagactttattcgcaggcaat |
| | | ggcgaagcgggcgttctcttagtaaaaacagatggggggaaccaataagcagaggaagtctgttactcatgaaaagaacaactacc |
| | | ctccctccaggactgggcttagcgtggtatgtcactgtcgatgggaagttgatgaatatattttcatatgccccgaggaggagata |
| | | atgaaatacagttcaatggaaacgccaaaaacgcgagaggaatttgaggctcgctgttttcacctgctcaatgcgatcaagttagg |
| | | acggtatcatggcattccgggtgaaggtaacaaagagcaggttccttttctccctaacggacgagttgatctggcaaacattgata |
| | | ccatgactcgcctctcgatgaactcgttatatgatttccactataacagggataattatccgcagtttgatctctctgaaaatgac |
| | | gagaatgaagaggctacggattgagctggccgatagaataatgtgcttggatcttagaggggcttccaaagaattagaacgctaag |
| | | gttgccaaagttgtgtacgaaaaatgattgatttggttgaacgctaaaaagaaagtgagtagcggtttgaagccaggctttcgagc |
| | | ttatataaacattctgc (SEQ ID NO: 41) |
| 45 | pLG047 | caggaagaagcattctattgacgctactatgttattagtgggcgtttgcgacagaatcaatggatagaattcacgggcgatgtagc |
| | | attttagacatctaagaagcactttagtcgataatctttcacctgttcgtctgtcaacatagatgcttgtgcgtggagtagtacgc |
| | | atacggccgagggctattgaccatagtgcattgtttgcttaacgttagtgcgtaggaaagaaataatctgggaaaagaattgaaaa |
| | | agatagaaaatattgcaacgtcgtgttaaaggcccgttttactggtacagggaaacaggcgctaggtgctggatgataatgacagg |
| | | aaatgacgatgctgaatataaggatgtatcctgcccggaatggtgatgcgtttttgctttgtgcagatagagccacattgcttatt |
| | | gatggcgggtatagttcaacgtttaacaactatattgtcgacgatctacggaaactggcttcagaggggcaagcccttgatctggt |
| | | gattaatacgcatattgatgccgatcatattggcggcatccttcgctttctatctattaacggcgcagcggcacgtcctgaaatta |
| | | tccagattaaacgcatctggcataacagtttacgcagtctgacggccccgcagactgagccggttgagcttaataatgaaattat |
| | | tttaaacacccttactcaacgcggttatttgacccccaatgaagaggggcagggcgccaaggctatcagtgcccggcagggcaata |
| | | cgctcgcctctctcattcatgacgggcaatatgactggaatgaaggcgacggattacgccgtatctcagttgagtctatgcctgga |
| | | atcaacttgcctggcgggcgcgttactgtactgacaccatcgaatacggcgctggatgcactactggtgttttggcaaaagagcct |
| | | gaggcgctttggatttaagggtgaggtgggggctgatacgctggctgaagatgcctttgaatgcggtgtgtcacacctgcaggagg |
| | | ccgtcgggaaaccaccttcgctaatttcagcaggtcgtcccaggcagcttgaagaagtttaccgacctgacacctctgtgacgaat |
| | | gccagttccattgcgacgcttgttgaacttgatggttgtcgcattttaatgctggccgattcccctgcagaagacatcgttcatca |
| | | gttgaaaattttgcaagctgagggctgttccctgctatttgatgcaatcaagatctcccatcatggcagttgcagtaatacaaatc |
| | | ctgaactgctggggcttgttgatgcaccggtgtattttatttcatccgacggcagtcgacaccagcatccagatgtggaggtgttg |
| | | acggccatcgttgacaggcctgccgctttttcccgcaccctttactttaactaccgaaccccgtcttcagactacttacaacatta |
| | | tacgacgattactggggcaccttttaccgtagaagcaggcacgtcctgctggattgagattggaaaacgccaatgatgctggatgc |
| | | ggaagtcaggcttgccacctgtaggattgcttgcgggaaagatacaggaaccggctggttgatatcacaggataaagtgctgacgg |
| | | cgcgacactgcgttgagaatgccctttttaatcaagcgcccgtgtctctgacatttaggcaggcagacacacaggtggaactgaag |
| | | gccacagtcctggatgaagatgaaaacacggacgtctgtttgctgttgcttgatgcaccgcaggatctgacccctgtacgattgag |
| | | tgaaactcgcccgttgccggggagctccttttatgcctatggatggcctcagagtaaactgggcatcgggcatcgcgtggagggaa |
| | | cgatcgcgcagatcctcgccgagccgctgctcggaatggatatagaaatagccatagagcagaatgcggtacttccccgctatgaa |
| | | gggctatctggtgcggcacttatcaccggggggaactgtacggggattttgcgggtttccattgagaatacggtgggcgtcatttc |
| | | agttgcagagatggcagcgtttctgcggcgtaacaacctgcttccggcacccgttacaccgacggagagttatgagaacaccagtg |
| | | aggcgcagcgggttgaattccggcacagttttgagcgcgttattaccttaaaacgcgggggatatctatttctggagggcgcgcac |
| | | ggtataggcaaatcgacgttttgtgcaaagtttacgcctaaagacccgacgattgagcattttgggacctatagctttaacacagg |
| | | ccgtgacggcgtgaatgcagttcagcaggctcaacctgagaccttcgttaactggttaagtatgcaggtttccctattcctgacgc |
| | | gggaacccgggcggcttatcaaaggggactactccgtactcatcaatgaagccggacaactgctgacgcgcctaggtgaagagtat |
| | | gcccgccgcaacaagacaggggtgctcttcatcgatggacttgatgaggttgataagtacgatgaggccctgcttaatcggtttac |
| | | agccctgttacccctgcagctcagtgaaggcttggtagtgatcttttctgccccgggctatacccgttattcagcacaactgggtg |
| | | tcagggtatcgcctgcggactgctgcacactgccagctctgactcaggcatcagcgcgggaatactgcagacagtcgctcaaagaa |
| | | gtaccatcgcaggggatgatcagggttatctgcgatcttgcgcaggggcatcctctgtatcttcgctatctgatcgatctggccaa |
| | | tgcgggaaaagcagaggaagagcttgctcagttaccgctcattgacggacgtatccgaaattattatgaaatgctgtgggttagcc |
| | | tgcaaaacaacccgctagtggttaatcttctggcgattatcgtgcgtttacgctggggaatttcacatgcgcagctcaccgaactg |
| | | ctcagtcttgaagagctgagcgtcctagtcagcacacttgaacgcatcagccaccttctgatgacccctggtgagacaaccattta |
| | | tcacgcctcatttgctgattttctggcagaaaaaactgtcctacgtgaagcagatattcagcagcggctgtctgcctactgtgaaa |
| | | gtcaccctgacactaggtatggccttctgaatcttatgtatcacagcctgcgctgcgacccgacccggcagatgtgggcaatcagc |
| | | cgctgcgatcagcactgggctgaccgctgtgttaccgagggggttaatccggcgttacttcttggcgatgttcgggaaacgctgaa |
| | | tgccgcattggcaagcggcagtctgacggataccgtacgccttcttctgttatcccaccggctgagctttcgctacaacacccttt |
| | | ttgcgcaatctgctttactcacagccagggcattgatccggattggccatcctcaggaagcgttgcaacacgttattcgtttcggg |
| | | cggctcagtctaccagtgacgcaagccctgcaggtggcgtttgacctgattcgtgcggataacgacagcgatgctcttgcgcttct |
| | | cagtctggcagatgactgggtggaggagcagctggcagaggtaaaaaccggtctttcttatccggaatttttacagctttatgata |
| | | tgcgtatgaatatctactttctcaaagggctggccggagacaggcgtgcggaaggagatttaaagcaatttcagctttactggatg |
| | | aacgtgattgagcaagtctgtgacgatgaggggacggtcagggggcttcgcggtcagatgtgtgcctcgttctttgcaggcatgct |
| | | gtttttccatggacgttatatttcgcttgcgaaactgagtgagaatttcacggggcccctgcaggaggtcacgcaatcgttcgtga |
| | | taacgttcatgtattaccattttctctgtgaggagtttcaggtcagtattgatccggagctgctggaccagctctttaaagacctg |
| | | acaacgctgagctgtctggaacatgaatctcctgtgtacgtagatccccggacacttgatgctatgatctcgtctggtgcccctgc |
| | | gcaaatgataagaaattttcagggggatacatcagtaccactgcaaccggtacgtttcattggtgatgataatgtgtcagcgaatg |
| | | atgtgtcgttcctggaggagatggctaaacataaaattcaggcattttgcgatccatcgtatgactgtccggcgcccgttgcgctg |
| | | acagcaactggctggatcgtaggcatggaggaattgtgtaggatggtggcatggtgtgagggggcggcaggacgttttcatttaga |
| | | gggagatgaagcagcccttgagtcggtgtggactgtcattgaaaagcaggtactgagcagcctgacatttccattatcagaccgtg |
| | | tggcatggcatgatgcctatgctcttcctgaagctattgtaccacagctttatgaacggctggcactcctgatatcgtctgttttc |
| | | ccttcccgactggacgcgcttttggcctttattgagcagcatttcccccgtcaatttgggctgtattcggaagggttccgagccac |
| | | gttactaaagattctaacactcctgagccaggtggtggatgacggtggaattcagaaccgcctttatgatctggccttccgttggt |
| | | atgagtttgtgctgggcaatctgcagaatcgccatgaacttgtgccagagttgttgcacctggtttcattatttgtccggctggat |
| | | gcgggtgaaagtgcacggcaggcttaccagcaggtgctggcattctcaatgggccccgactggtataaagaggatcagtttggtct |
| | | gatgataacagcgctcaagtcaatgagcgaggcggacgcgatccctcagcgtttgctcgcccgtattgcctgtctgctggatatgg |
| | | ctggcggtgagatgacctttcagcgttacgtgcgatatgcgcgccgtgatttcactgcggcgttgtgccagcacggtaatttctcc |
| | | caggcagccgcgtattttatgtgtcaaacatacggtacaacagctcagctttatgctgaagctacgcatggcgacatcgatcgtgt |
| | | gtcattactgaaaggaacgcgtttccccgggggcgcactagatgaacaggatgtgatcctgaacattgtgcgtttcgctgtcccga |
| | | tgtgtgactgggcgttatgctgggcattgcttgagacctaccattttggcgatgcgcgtcatcttgataattatgcagatgcctat |
| | | gctcaaatgatgatcaacatgcaggactgtcaggatgcaatggcgatgatcgcacaacggctcacgcttatttttgaagctgaact |
| | | gatgcctgggaaccggcacctgtttatgaaatacctgcgaagcgcacttcctgaggctctcagggataaaactgattttctgaacg |
| | | tttacctttcagataacaacagcgccccagcacagcagagcgagccatttgaagacgtcgcagaaacgcagcatgcaccgcctaat |
| | | gtttttgcaagggcatcgcttgcgcttgatgaggctgaaagtcaattgcacagacgtaacacgtcacaggcgcagcacaaggcaat |
| | | caatgcacttgagatgtttcagcaggagggatggtcggtatggagcgacttatcagaggagcatagacgtgcaggctccatactgc |
| | | tgaaaagcacggattcggtgtcggaggttgtgacgctgagtagggcgttaatttctgcagagcagcatacggagagctggcgtatc |
| | | gctgacaagctgattgaatggttgtctcctgcagcggatgagagtgtacaggctgagctggctgagcattcgctatcacacatgga |
| | | gatactaaccggcatgcctgttgccgtcatcgaacggtatgattttcttaacaggaaagaggatcagcatccgtcttctgcgctta |
| | | cccgtctgcttctgcatgctgttgatcatcctgtctggatgcgcagtgagaaagctgcggatatgttgctgtggctgctgcagcat |
| | | catccccattacgtatccgacgttgggcctctggcattttcaatggtttcactgaaccatccggatgtgctgtgcgggatactcga |
| | | taagctttctcaggatgatcctgggtctttatggactttgctgtcagcacatctggatgtggcagagacaaaaaaatcctgctgtc |
| | | atgctggccggctcgccacattagggcgaattgcgagacgggctgcatccttggggaacgcgagtgctgctgaggcgctagcgtta |
| | | ttgcatgacggggaagtacgccagcccttgcaggaaaaaatcgcacagcagagtccagcgtgtccaaaatgggctgagataattgc |
| | | ttttcagtggcgacagttagcggatgccgggctggttgacggcagcctgtcagagagggcatttgctgtgctgtgtgaggcgtgtc |
| | | atcccttcgggtgggaaacagtagaggctcttgaagaacttttggcgacgggcatgagcggaagcacggcctggaacggccgatgg |
| | | gaggcaaaacttcgctttgccttacaggtagcacttatgtccgttctggacgatgcacagtgccttcaggctgaggctattttccg |
| | | tatctgtaatcctgagccgactgacacattcagaattacgcatttttcatcgcctggtaagcaatggctcaaccagttgatgcagg |
| | | ggaaggttaaattttcacctattgctgacagccagctctatctcgatttttacgagaggcggaatattaacggcgtactcgttctg |
| | | ttgaggctgacggcttatttctaccgtgacggggtagatgctccctgcttatccggacgttttcctgcaaccgctcttgccacatc |
| | | tgtgcgggcaggccaactggacacatgcgtgaatgttcaagcgacgcctgcatattttggcagtttcacgccagcaattccttctc |
| | | aagggctaataacgctcactagggctctttcgcatcattttaaacgagctagttggcgaaaggggcgggatgttgagagtcagggg |
| | | ggcgcgcctctggaagaagggtgttatttatccattaaacgggacgcgttcagactcccgccgggaataagggttgtatgggtttg |
| | | tgaattcaacaacgaaccgattgcgcttatgaacgccgctggcgcactgaagattcactaggaagaatatgaatataccgttaacg |
| | | cgaagtgaattcgagcaccgacttcatctgcttgagaatcattcaaaaacgggtcggctcatgctggcagagggggtatccggtga |
| | | gagtttgcttaaagtcaggcgactgccaaacggccggattgattttctctccgtggatgaaactgcccgtcttcaggcgaatatga |
| | | tggagtggatgaagtcgattcccctgccgaacataccgaacgatgagggcactccctaaacttaagtatcgagttaatcctagtag |
| | | aaggggatgtgaaaagatacctttgaaaggtgcgaggtcaatggaacaactttcagagatttatctcttatctgaatgttcatcac |
| | | ggagctgcgttgtagtggccccgaaaaaactcactatagagaacggtctaggagaagactgtaaaagcatttgcttgcgttaattcg |
| | | (SEQ ID NO: 42) |
| 46 | pLG048 | gaaatttcgcgacagagatccttaacggtgcgtcgagcttcgacggaattcagaataatgatggtctggtgttcggtgaatcgtgc |
| | | tttgcgcatggcgatctcctatcagaacaaaaccagtatgccggatgatctctaaaagtgaatggaccgatatgcagggatgctta |
| | | cagtgggtcttcgacctttataagcatagtaaagaatagaatatgccaatgtacgataatctgtgcactctattacctgcgcaaaa |
| | | aagtacaccagaattgtttgtctggtttggcaaattgagatcattaggcggcatagcgaatgactttaaatgaaaagcccgattca |
| | | tcaataaagattgttaaaacaaaaaccttgcccccagcagagggcgagcgccgggcaatgcgtggctatatgggccaatatgaaag |
| | | agccggtgcagccatttatgctgaattagagcgtgggcaattggagtggataggcgtagcggaccgcagtgcgggtatcgttgatg |
| | | atttagtacttggatttaatggccttatcgttgggcaccagttcaaaacgtcccgtttccctggtacatttacagtacagacactc |
| | | ttagtagggtctgatggtctgcttaagccattagtttgcgcctggcaaaatctttgtagtgctaacccaacgtctcaggtagaaat |
| | | tcgtttagttgtcaacgattatccatcagttaacgacgctcccggaatggaagctccagctcatagcgctgccttccttgatgagt |
| | | ttgaacattatcccaaacgcacgcttgaggaatggcgctacagtaactggggccgtttagtcgaaatattatttcaacattcctgc |
| | | ctaggtgacgatgatttcgagagattttttcatgcgttgcgcataattcatggttctgcagcagattttatacaattccataaact |
| | | cagtgcagaacaagcgagactggcgtctgatatagcaaaaatattacctcgactggtctccgataaacgagatagggatcgatggt |
| | | cctgtgaagaactattatatgaactagggtggaaagatcccaccaaaacacgccacttacatcgttttcccatcggtgctcacgtc |
| | | caacgcaaccgcgatacggaactacaacttctccagacgatacgcaacacaatccagggctatgtggcattgattgggcctccagg |
| | | ttcggggaaatcgaccttgctacagacaaccctagctaccgagtataacactcgggtcgtgcgctatctggctttcataccgggcg |
| | | ctgcgcaaggtgtagggcgcggggaagctgatgatttcttcgaagacatttctgcccagttacgcagcagcgggctgcctggactt |
| | | cgccttcgagacagcagccaatttgaaaggcgcgaacaattcggtgaactgctcaaacaagctggcgagcgttatcaacgtgatac |
| | | agtaagaaccatcattattgttgatgggctggatcatatcccccgcgaagaactaccagcccattcgctgttaggggaattgccgc |
| | | tgcctgcagccatccctttgggcgtgacatttatacttggcacccagcgactggaactcaggcatctcaaacccgcagtacaggaa |
| | | caggctgggcatccggatcgtctcgtaacaatgcatccacttgagagagtggcggtcgccaggatggcagacgttttaggtcttga |
| | | ttcaaccatttcgcgtgtaaaactttatgaacttagccgcggtcatccgctggcggccaattatctcattaaggcactgttatcgg |
| | | ctgatgaacaggacatatcatgcatcctcgccggagggatggaatttaatggcgatattgaatcagtttacgcatctgcctggaga |
| | | gaaatcgcaaacgaccctgatgttatgcatgtactgggtttcattgcccgtgtcgaagctccgatgccgctgaaattgctggcaac |
| | | aatcgtagatgctcaggcgatagagcgtaccttaaagaccgtccggcatttactcaaggaaacctcaaaggggtggactgtattcc |
| | | ataacagcttccgtctatttgtgctctccaaaccaaagataacactgggcagtatagatgaaacctattcacaacatatttatcgt |
| | | gaattagctaaactatctcgtcatgcaccagaacattcattacagtcctggctaacactgcgctatctcgcccggtcaggagagcg |
| | | tgatgaacttctggcactcgcaactccagcatattttcgacaccagtttgcacatggacgttcctgttcagagattgatgcggaca |
| | | ttcacttggctctgattgctgcgcgttccacgtatgatggtgtaattgccacacggttattactttgccgtgatgagatatccaga |
| | | cgaactcaagcactggagtatgccaatgaacttccgcgcgcgatgttaaaagttggcgatattgatgcggcgatctctttcgtcca |
| | | ggactttcccaatgcgggctatgaagttgttgaccttcttttggaacagggtgattttgaccgcgcgaaagaactgtttgagcacc |
| | | ttgagccattatctcaattgcatacccccagattcgagcactatggggattcgcataatctacaagaattcaaaaaatgggcaaaa |
| | | cgagttgttcacttccgcgacgctgagcaaattaagcaggcaatagactatttgaccgttgaggggtttaaacacgccacaagtgt |
| | | atcaaccgatgaaaatatttcctctattcgcgaacagttaaagtggacagtggtcgaggcaattgttaactggcaatcagacgtta |
| | | atattcaggatacctgcaatcagtatggcattcatgtgcaagagataccggttttgatgactcaggctggatttattgctagagac |
| | | agaggaaataacaccttagcatcggaattatttaagactgccatggcattgtctgattttaatgatgtttctaatggggggcgaag |
| | | atcgattgcattattttatgccacatcaggctgcaccgatctggcttcaaaattattcgaaaacctttttgcgcctgcaatttcga |
| | | tgggagacaatgaattagaatcaacaaaagcactgacgcttgcagccatggaacatgcgcaactttgcgttttgctcggcaaatcc |
| | | ttgcccgacgtagtcacctcaacacacgctatcttacgaccgctgcagacacatgcttcagaaacgggacgcttgttggggctgtc |
| | | cataataaatgcctcatgtattccttctggaaatattaaaatggtctgtcgcatggtgatgagatatgtaatgcaactcaatagct |
| | | attctggaaacgatacctatcaggctcaattggcattgacagctacatcaccactgatttgtacattaattaaaatttctgcgctg |
| | | tgtggtaaggttgaatattattcagtaataaatgaaattgataatgcaatgcctgctttaatattaaaaggcaatacactactccg |
| | | gcgtgaaatagcattggcaatgtatcaggctgacggtgaccgtgaaagggcggccgccagatttgagcctatggtaaacgagttgg |
| | | tagaaaatacacctagcgagcaactcgagactctgtcagttctggcaaacagctttgctgcaattggcgatgttgaccgggcacta |
| | | aacttacttgcttcgatacatgaccactgtttaggctacgctctggcagcgcgtaaggaccctttatactctgtttggaaagacat |
| | | attgattttggccaatgcggcagacccagaacaccgtgctcaacgaataggtcagttgatacgacaggttgatggtatgaaggaaa |
| | | ccgagggagcatctgccgcatatcgtttgacagaagtgttaatcaatgaagcaatgcgtatgaatgcgcacagtggttataccgtg |
| | | gcacagaaactcagcaactgggggctgattccatggccaaatcaggtaaatgaactggtaattggtatgctagatcgccgtcctga |
| | | aatggtgtttctctgtacacaaatttggtgcgggctatgccttccattctacattgaaccctattatcgtgaccctacacatgtag |
| | | gcaattatattgacgttgctgcaaatgcagcggggccttcatcaattgccaaactggtatcaattctattaccggcaatccaggtt |
| | | catagtcgagctcacgagcgactcacgctaataaatcgcctgagcaaggcggcattaagacacggttataccgataaccaacttga |
| | | taatgccattactcgatggacttcagaggcccccgaagcccgccgctcctacacgccacaaacgtacgacgaagcttcaacccttg |
| | | acgaacttcaacaggcatttgaatcaaatgattccgaacctgagtatcatgcgccttatcgtttttgtgagcttgcagagtccgcc |
| | | gcattagacaaggtggtgaaaatgtatgagtgctggcattgcctgcagtcggatgcacgttgtcgttttttggttgcagagcggct |
| | | agttaatgcgggggacacgacgttagccagaaaattagttgatgattacgataccagtagtgaccgggagatgtcatggagccaat |
| | | ggttaggaggaaatcgattccgtctcttccacgcgcgtaagctactcgatggagcagcaattcatcatgaagcatatgaagacttc |
| | | atcagttcaattgtggctgggaaagagagcaccatgtcgttgctaacagatatggcagacattcttcctgtgatctgtgagtcgcc |
| | | agactggcccgccgtctggtctatcctggcagagcagatgtctttcactcgcgaacaccgtattggtgaacttttcgaatttggaa |
| | | atgaaaatatgaccgacgaagagttacttgcggaattgctccatttttcattacgattgcctatcaccgaagctcgacgacacgca |
| | | gagaaaactgcactaattctggcggtacattcaacaggagggcaaatcgtatttgagaacaccataacacgactcctgaacggcac |
| | | ccttgatgaaccattccaggcattgcaaattttgcttttgctaaaacagaaccactttgctgctaaatttggtgatttagtctctg |
| | | gccttacgaatcatcgtgatgtagctgttgctgaagctgcgtgcttgttagcacaatattggcagctacctgtatcgattgatttt |
| | | catccgttgccgttgacctatcgattggcactcgacggagaccctgatcatgaaaatgctctgttagatcctgtgagtggggcaat |
| | | gcgtattgaagtcgacttaggatggacacaaatgcttcgtcccgttgcacggagacttgcagagtttgctgattgtgacgaaatga |
| | | acatacgccagcgtgccgcaacgtttattcagcaatggggagggctggcagcctttggccctggagcaacaaaaaaaatcgaatct |
| | | cagttacgcacactctcaatgcaaatcacctatcttaagccccatgcttacattggcatactggcacttcgtcatgtcgctggaga |
| | | gctgagcttggcaggcttgctctcgccaagggataaaccatcgctactggaacaaatggatgcagtacttccgccaactcctcgcc |
| | | ctgaaatgcaaatccggccaactggcattaggcgaccgcttaaagtcaaggatgccccgtggagtgaagctgaagaaatgtggaca |
| | | aatttggttgacgaggatgttaaaccctggataggtcgtgccgacgaattcgtaatagccgaggtttcacaattcaaaatgcatga |
| | | tacccggcgtgctgaatatcaggtctatcgtattagcgcacctcaaattcatatttctgatgccaaattcatggcatggtatcaaa |
| | | gtttgcccgctgtcgtttggctgggaaaaatgatcccacttgacgaagacctcgcaccgacaatagtcaggcgtgtagtaagctcc |
| | | atcgggacaatgtcttcgccgggatatgccattgcattatgtcctaatatccagatgcatctgggatggcatgaatgctgcgagat |
| | | gcctaatatttataccgaccagaactcaacaatcgtagcaagattagtgaactggcgagacgccgggccagtggatattgatgatg |
| | | attatatatggggggaaggttgctatctgacgctttccaatgcaggcctgatacaagtcaagactctgttcggcgaattcaccgtg |
| | | cgtaatttcgcaagcagggctgttcggcaattgcgacaaggcgaagcgcaaatgataaagacagctcagaatcagttcccgatact |
| | | gtagcgagacgatttcacaacacggttcgattacctgacttctccaaccatggtctgaagaagtcagggagtgtagatcatgccgg |
| | | cattctgtttctgaatggcgcaggatttcgggtcagggtcaccacaacaggcttgtccttttct (SEQ ID NO: 43) |
| 47 | pLG049 | acaattttttgccataagacgctttcctgaaactcttctcattctcagcaggaaagcgttctcttctcaatactctctggttatag |
| | | agtattaaaaaataaggagttataatccttgtagcccaactgacataaggacgatgctcaatgtctgacagcctgcttgttcgcac |
| | | cagtagagatggcgatcagtttcattatctttgggcggctcgccgcgcccttcgactactggaacctcagtcaactcttgttgccc |
| | | tgaccattgaaggggcatcaacgacggaaatgggctctcagccagtggttgaggatggggaggagctgattgatattgctgaatat |
| | | tacggcagtaacgagctcgcaacagcaacaactgttcgttatatgcagctaaagcattcaacaatgcactcagatactccatttcc |
| | | ccctagtgggttacaaaaaaccatcgaaggttttgcaacccgttataaggcacttatacaaaaaataccggtagaaacgttacgca |
| | | ctaaactcgagttctggtttgtgacgaaccgtccagtcagtagcagcttcagtgaagcgatcaatgatgccgcgaaccaacacgtt |
| | | acacgccatccacatgatctggcgaaacttgagaaatttaccgggcttcaaggcgctgagttatcgatattctgccagcttttaca |
| | | tatagaaggtcagcaggacgatttatggagtcagcggaatatcctgctaagagaatcagcgggatatctccccgacctggatactg |
| | | aagcccctctgaaattaaaagagctggttaacagaaaagcgttaaccgaaagcgccgcaaatccttccattaccagaatggatgtg |
| | | ttgcgtgctttgggggtggatgaaacagatctttttcctgcgccctgtcgtattgaaagaatagaaaattccgtctcaagaactca |
| | | agaggcgacgctggttcaacgtgttgttgaagcattcggcgcacctgtgatcatccatgccgatgccggtgtggggaaatcaattt |
| | | tctctactcatatagaggagcatcttcccactggttctgttagcatcttatatgactgtttcggactgggtcagtaccgtaacgcg |
| | | tcttcctaccgccaccaccatcgtacagcattggttcagatggctaatgaaatggcatctcgtggtctctgtcatccattgatccc |
| | | aaatgctggtactggcatatcccagtatatgcgtgcgtttctgcatcgcctttctcagagcatttcaatactccgggcctctgagc |
| | | ccttggccgtattgtgtattattattgatgctgcggacaatgcacagatggcggcggaagaaatcggtgaaacgcgttcttttatc |
| | | aaagatttaattagagaaaagcttcctgatggagtctgccttgttgcactttgccgaccttatagacgggaattacttgatccacc |
| | | tcctgaagcactcacattatccctacaaacttttaatcgcgatgagacagccgctcatcttcaccaaaaatttccagatgccagcg |
| | | aaagtgatgttgacgagttccatcgtctaagctcttgcaacccccgggttcaggctctgtcattatcacaaaatcttccactgaac |
| | | gacacattgagacttttggggccaaatcccaaaacggtagaagatactattggtgaagtgctggaaaaatccattgctcgcttacg |
| | | tgatacagccggaatatctgaacgtgctcaaattgatacgatttgttccgcactggcaatattgcgtccattaattccattatctg |
| | | tgctatctgccatttccggagtagctggttctgctattaaaagtttcgcacttgatctgggacgcccgttaatcgttagtggcgag |
| | | actattcagttctttgatgaaccggccgaaacatggtttcagaggcgctttaggccatcggccgctgatctgcatcagtttattac |
| | | taaactgagaccactaacaaaagatagttcctatgcagcatcagttttacctgcattgatgctggaaggaaaccagctttctgaac |
| | | tgatcgagctagcgatatcctcacaagctctgcctgaaaccagcgcggttgaacgcagggacatagaacttcaaagattacagttt |
| | | gcgttaaaagcagccttacgcacaggtcgataccaggatgcggctaaactggcactgaaagctggtggagaatgcgcgggtgacaa |
| | | caggcaaagagtcctgctgagggacaatatcgatctggcagcaaaatttgtgggaagcaacggcgttcaggaactggtttcccgta |
| | | acgcatttccagatactggctggcctggctccagaaatgcttattatgccgcaatactttccgaatatcctgaactctcaggagag |
| | | gcccgcagtcgccttcgactcaccatggagtggttaacaaactggagtcaattaccagatgatgagcggagcaggcaaaatgttac |
| | | cgatcaggacagagcggtaatgctcattgcctgcctgaatattcatggcgcggaagcggcagcaagggagctcagaaggtggcggc |
| | | ctcgaaaactatcttttgacgctggaaaaattgttgccatgcagttactggcccacgcccgttatgatgaacttgatcagttggct |
| | | attgcggctggaaacgatatcagcctggttatgggaattgtactggaagcaagaaaacttcaccgtccagtcgctgaacaagcaat |
| | | cagaagaacctggcgcttgttaaaaagtcagcgagtcagcattaaagacagaaaccacgctaataaccagacaatagcagcaatca |
| | | ctggcatggttgaaatggcgcttatccaatctgtttgtactgaatcagaaagcatccagttgttggatcgttatttaccaaaggtt |
| | | cccccctatgctctgacttctgagtatagtaaagaaagagttgcttacgtccgggcatatgctctgcaggcaaacctgatgggctc |
| | | tcaattagcgcttagcgatttagcctccacagaggttaaaaaagaacttatggctgaaaaacgccacggcgaatctgatgacctgc |
| | | gtcaactgaagcagtacagcggagtattaatcccttggtataatttatgggccaaagtaattcttggtaaaacaaggaaagcagac |
| | | ttagaaagtgagctaagtgatactcaaaaagaatcgacggctattaaaggtcattcttactctgagcattcattatcatcaaatga |
| | | gatcgcaaatgtatggtttgatattctgatcgaagcaggtaatgtatcaaaagacgatgtggaaaacatcatcaaatggagtcagc |
| | | ataaagggaatagagtattcacaccaacgcttcaccgtttcagttctgtatgtgcagagatttcagggcttggagagctttcatat |
| | | cacttcgcagaacttgccttatctttatggagggatgagcactctgatgctcagatcaaagctgacggctatatagacctttcccg |
| | | ttcactcatttcacttgatgaaccagaagctaaagaatactttaaccaagcgattgaagttacaaataagttaggcgatgaaaatt |
| | | taagtcgatgggaagcgatacttgatcttgctgaatatgttgctggtaaaacgcaagtccctcctgaaatttcctataaactagcc |
| | | cgatgtgcggaactaaccagagaatatgttgatcgtgataaacattttgcatggagtgatactgttgagattttggctgagttatg |
| | | tccatcttcagccctagcaataataagtcgttggcgtgaccgtacatttggcaatcatagaagcatactggcatggaccattgagc |
| | | atcttgtaaagaaaaataaaattaatgcactcgatgcacttcctttaatcacatttgagaatgattggcataaatgcgacttgctt |
| | | gattcagttttatcctcgtgtactgatgacaaagataagatcatggcattcgaagtggtttaccactatacaaaatttaacgtaca |
| | | aaatatccaaaatcttaaaaagctggatgctatttctacatcattaggtattgaacacacagaactgaaagaaagaatttcaggtc |
| | | tacaacatactgagacggtttcaaaaaaatccagtctctcatcgaatgataatgagcaaggccatgaccaggaatgggagtccatt |
| | | tttaaagattgtgatttatcgtctattgatggtattagtgcagcatacgaaaaatttcgtaatgttcctgaattctattccaaaga |
| | | aaccttcatcaagaaagcaataagccgagttaagacgggcaaagaatgtagtttcattactgccattggtgctatatttcactggg |
| | | ggctttatgattttaaatatattcttgaatctatacccgacgaatggacatctcgtttaagcattaaaaccaccctggcaggttta |
| | | ataaaagaatattgccaacgcttctgtatgcgaatcagaaaaagtcgcgtttacgagatttttcccttcagtctggccagcaggct |
| | | ttctggtataagtgaaaaagagattttcggtattaccctggaggccattgcagaatcgccagagcccgcaaactctgaccgtttat |
| | | ttagccttcctggccttcttgttagtaaactggagagtaatgaagcgttagatgtattatcttatgccttggatttattcgacgag |
| | | gtgctaaaagatgaggatggtgacggcccatggaacgagaaattatctccgccaactcatgtagaggattcacttgcaggctatat |
| | | ttgggcgcggctgggttctccggaggcggaaatgcgctggcaggcagcacatgcggttctggcactatgtcgaatgagtcgtacat |
| | | gcgttatacaaggaattttccagcacgcaataaatgctaccactttacctttttgtgatcgcaatctgcccttttataccctccat |
| | | gctcaattgtggttgatgatcgctgctgcaagggttgcgctggatgatggaaaatcgctgattcccaatattggttatttctacca |
| | | ttatgccactactgatcagccacatgtattaatccgtcattttgctgccagaactttacttgcactgcatgatagcgacctgatct |
| | | ctatcccagcacaagaagagaataaactccgaaatataaaccagtctacgactctccctgtgcttgataaggttgaagatcataga |
| | | ggtgaagattcatatacttttggtatcgactttggcccttactggctaaaacctctgggacgttgtttcggtgtatctcaaaaaca |
| | | gttagaacctgaaatgcttcgcattattcgtgatgttcttggttttaaaggtagccgcaactgggatgaggatgagcgtaataaac |
| | | gacgctattatcaagacagagataatcatcacagtcatggttcctatccacgggtcgatgactaccatttttacttgtcataccat |
| | | gcaatgtttatgaccgctgggcagttattagcgacaaaaccattagttggtagtgactacgacgatgtcgaggatgttttccagga |
| | | ctggttaagaagacatgatatttctcggaacgatcatcgctggctcgccgatcggagagatattccccccaaagagcgctccagtt |
| | | ggcttaatagcagttctgacaatagggatgaatggctagcgtcaatctctgaaaatgtatttaacgaaacactatgtcccagcccc |
| | | ggactattaacgctatggggacgttggtctgacgtttgttcagatcgaaaagaatctattattgtccattctgcgttagtatcgcc |
| | | ggagcgatctttatcgctcctcagagcattacaaacaactaaaaatgtatatgactataaaatccctgatgctggagataatcttg |
| | | aaatagatcacgcacactatcagctaaaaggatggattaaagatattgctgaatactgtggaattgatgagtttgatccctgggca |
| | | ggtaatgtaaggtttccaatcccagaaccagcctcatttatcattgatgcgatgaaattaactactgataaagatcatcgggtatg |
| | | gtattcaccttctgatgttgaaccggcgatgatttccagtatctggggccatctatcaggtaaaaatgatgaggaaaaatcacatg |
| | | gttataggctatgtgcttcaatacacttcataaaatcagcattagaaacattcaacatggatctcattttagaggttgatgttgat |
| | | cgctattcacggaacagcagatatgaacggaataatgaaaatgagctcgacaatatcccttcaagcactcgactcttcctcttccg |
| | | acatgacggaaccatccacacgctatacggcaattatagaaatggggaaaaaactagttgatgagcttgagctaaatgactctgtt |
| | | gatacattaagcagatggatggctcatcatatcgcagagctcatttatgatgctgaacattgtacagacgacatcgtccgtacagc |
| | | taaacaagcggagattagggactctatctggtcattctggtctaacagatacgaattgccaattggtagcagaccatttcaggagc |
| | | tcgaacctattctaagaaccttaaaaggtcttgatcctgaaaatgagcaaccgagatttttttcaccttaccgagatctaattaat |
| | | gtagaaaaagaaaccagtgaggtccaaaaatggctaaccgccgctaaggatattgattcagcagcaaaaatactgattgattactg |
| | | tttatcgttagcagcagaaaatgctatcgataaatcccaagaatgggtggaattagcacagaaagctggattgaacaaagatgttg |
| | | atctgcttgaaattcgtatctttcagttacgaggtaccccagccaatacagacaatcccaataatgcacaacggagaatactggaa |
| | | aaaaggcaaaaaaggcttgaagcttttctcttattgggctcccagttaaacgaacaactcaaatctcagcttgaagccttaccagc |
| | | aattgaggatgagccaacggatgacgacgaagacttttgatatgacttgctttagcactggagacggctcacaagacggaccacat |
| | | aatagcctaacccaagacttttctactagtcctaatg (SEQ ID NO: 44) |
| 48 | pLG050 | ttgtgcgtagcacttctccagtttttgttgaaacagataaagagactaaatcgatcattcgaacccaaaaatggccgatttgatgc |
| | | agacaacgatttaagccatatctggtagcgcaatcgtcacctatgacaaaagttacatacttgtaatattctgaattcaatattct |
| | | tcgtgaaattcattcaatgcttctttgagtagtgttttggcgttatgataatttcctaaatatcataaggttatcaggcggtgatg |
| | | tatgaggcgatttgtctatggcgattaaaaacagcgcaatcatttatgcaggctatgattatcagacactccaaggtgtcaggcta |
| | | ctggcggattggctcaatacaccaactaaatataaccgaatagcatttgaggctgatgcgaaacaagttgatgctccacaaggcat |
| | | tgatgatattgtctgcgaacgtcaggatggtaaaacagatttttggcaagttaagtttacgccagataccgacaaagaagacaatc |
| | | aactatcatgggaatggttactgaaacgtagtggtcatagtattcgagctcgttctatactgcaaaaaatagctgatgctgttgat |
| | | aaagtacctgcggaaagaaggggagatattactcttttgaccaataaaatacctaatcgtgagatagcaacttgcttgcgaaataa |
| | | caaaatagattggaatcaggttccaattgctaagcagcaaagcattattcttcagttaggtacccaggaaagagcaaagcaatttt |
| | | tcgatatattacaaatatgtcatagtgatcaaagttatacgcgattaaatagtattgtcccagaactacttcgcaaacataccaac |
| | | gaggagggggtatatcgcctgattgaacgagctaaacgttgggctatccagcgtaattcaccttcggatggtggatggatatgtct |
| | | tgaacatattcgtgcagtgatttcaactaatagacctgaacctattccgcagacttttgtcttgccagataactatattgttcctg |
| | | atgcagattttcacgacaaattcattgattcactttttaatcctactaatcgattagttgtcttaactggtgctccaggaaagggt |
| | | aaaagtacttacatcagccatatttgtcagatattacaaactcgcgagtttccttatattcgccatcattattttcttgggttaga |
| | | tgatcgtacgacagatagattaagtcccagaatcgttgctgaagacttgatgtgtcaggtcaaagcattttgctcacaaatcgaaa |
| | | tgaaaaattatcatgcagagcacctacataaagtgctggctgaatgtgggcagatatataaagaagaaggtaaacgatttttcatc |
| | | attattgatggtttggatcatgtctggcgtgataacggcaaagataaatctccactggatgagctattttgccaattgttaccgtt |
| | | gcctgataatgtaacattattggttggtactcaaccagtagatgatgagctattgccatcaagattgttacagaacagtccaagag |
| | | aagaatggttgcacctaccaaatatgtcaggcgatgctattcgtaaatatctatcgggacaagttgaaagtggccgtatcgtattc |
| | | aattttcatcaaagccagtatgaagaagttttatcacagtgtgctgagttgttgactactaaaactcagggatatcctcttcatgt |
| | | tatctactcatgtgaaaaattacatgttgaaggtaaagggttatcgcactgggaaatagaaaacctgcctcgctgcgaaggcggaa |
| | | acattacaaattattataatgaattatggaaaatattaaattacgagcaacgcgatattcttcatctctgttgtgcttttcctttt |
| | | ttatggcctgccacatcattttctgagattttttctgagaggactgaaactataccgaatgttaaggctgtaatccatttgcttta |
| | | tgagtccattgctggattaagaccgtttcatgaaagcttgattgtttttacccgtagcacaactgaacatgagaatagaataaaat |
| | | tattattgccagcgctaatttcatggctggagaaaagcgcacccaaaccgataaaaaattgttggtactggtcatgtcttgcttac |
| | | aatggtgatccatatcctttaagaaatggcttaactagagactggatattggaacggttggctgaagggtatcgacaggatgagtt |
| | | tattcgattactcactcaggctgaaacttctgctttagccgaagggcattttagtgaggcctatcagcatcgttcacgcaagactc |
| | | gactacttaatgctaggttgcaaatctgggatatgtcgacgttgggcgtttgcagtatgattaatgcttctgaagcattgcttaaa |
| | | caatatcaatctacccagaatgtcagttcaccaaagatactggcaactttggctatcgctttatggtttcgtaatcatttcgatga |
| | | agcaaagcgcattacaagattggcgttacaacgctactcaaatgaatcatccgtatataccaataaaaatagcgatgagtcgcgtg |
| | | ctgacattcgtttattaatcaaagctgctgttttgactgagtgtttcgatgaaaaatggttggcaaccggttcagtacacaagtgg |
| | | agtgatagtaatattaatctgcttatcgaatgtgcggaatataaatcagatataggattactattttcattacatgatgtttttaa |
| | | gcaaactgtcataaaaaataaaatagtaaatgcgattgtcagagttgggattgttgaacaaatagatttagaatactggccacatt |
| | | tttctggtcttgactccgctctgctgcggttatacagtcatttatccactgcacatccatgttcacttataacagagcaaggtgaa |
| | | agtgaaatcggtagatatcatgttcatccagaagtatcctacgatgaatggttctatgacagccttttttatcgtcttaatgccag |
| | | tggagattattgttggctaccggttagcacgggggaaggacaggaggaagtcagcagtcattttctccatttaaatgatttctcag |
| | | atattattgctgaaagtatggctctaaatattcaacaaagcttcagcgatttttgttcacttattgctttggtatcagatcttaaa |
| | | gatcatcaaatgcaaatccaacagaagcgaatgttttttaaaactgattgggtaagcattgctttaaatttacacttaatcatgca |
| | | ttgcaagccggttaatacggaagaaattgatattattcttaattctgagcatacagccctgtatcggctgcataaaactattctta |
| | | actttcatagtagagccttcgaatctgatgcaatagcaaactttctggtatttgaggatgggaggcagaaggaaaaactacaagag |
| | | acaaatgaatatttggcgaataatcttgagttgtcagagattgcgcttcattatgatctcaatcaatcaattttttttgagcgagt |
| | | caagttatgttgggactatggtctgggatacggacatcataaagatatagctctgaatcaggtgctgactgcaataaaaactattg |
| | | caactgttgagcctaaatatgcattaacgcagcttgagcgtgtgagtccattggttcataatatttgtgacttcacagatggtgac |
| | | catactcaacattccgtaacggaattgtctgcgctatatgctcatctttctccccttactttaagtagtatctatgacagttatgt |
| | | tagcgagggtgagtggtatgatgcggataatgcattaacgcaatacttaaaacatgctgatctatcatcacctttcgttgagagtt |
| | | tatgccggacattactagatgatgggcaaattgaaataatacagaatcgtgctaaagacaatgccatattgactacgttttggccg |
| | | gaaatattaccacgaaaaatggattatagtagtagcgcaaaacgttcattaagggggactgaaaaatttgatccagcaaaaatcag |
| | | ccctgctgatgtaactaatttactcaatgttcggtcaagttatgaaaatattcctaagtggtatcattattggaaagaccaaggaa |
| | | aagttacagaagtaattaacgtattgctgccaatcattaataatggcttgccagaatatagtgaatttcgttatatattatctgat |
| | | ttatttgaagatacattgcgtttgaaaggtaaaaaatatgcttttcccattttagtgcaggaacatattcagcgaaatggttgggg |
| | | tgaatggggggagtctgatgatcaaacatatgctcggttagataaagttatcagattgtatccggataaaattgatgactttcttt |
| | | acaagacgactcgacttcatcactataaaactaaagaagagaacttggtaattcccgggaataagctaacatatttattagtaaat |
| | | gtaggccgagtggatgaggcgaaaagtctatgtgaagcgatgatttcggaggtagaggcagaaacccagaatcttccgttgtgcaa |
| | | acctcaatggcaatgggagggagaattagataacgatatgatcgccgttaaattcatcattcgtcgtcttttttggcctgttcaat |
| | | gtgtaaaacatcttgtcgctgatcaattgtctcatctcttagttaatggtcaatgtgctgaagaaattgaaaatttacttgtagtt |
| | | gagatgggaaatcgtcaactggagtcagaggtggtagatattttaactgttctctggttagctagtttgaaaggttataaggttca |
| | | gaataatatatcttcctttatttatgctcgtagctttctttcagatgcattgctggaggctatcgttccaaatttaccaaacctca |
| | | gtcgctatcaagtgctgtataaacatcctgatgatgatggtaatcactatggctttgaaaaaacacttggcaatgaacttccccat |
| | | atattttgggatgaagtaaaaaggcttgaggagaaatctggagctccggctaaaatattaatgaaaaaagaatggaatgatatttg |
| | | ttataatcatgttcaacgatgggaaagggttgattatttcttcggttcagagcgtgatggttttactatgagtttttccacaagga |
| | | atacacgatttggtatatctgcatacttgagaaccattaaccggcttatcaacgaatttagaatgccaaagcattatgcagaacat |
| | | tattcgatttgtttaatgtcagccaacccattattttattccgtatctaatcaccgacctggttggttacctttatggcaatatgg |
| | | ggagattaccacaaaggaaaatgtaaaaacatatgttgaggaatgcctgaatgcattcaaaaatgaacaggaaaattcaatattag |
| | | gagcattgtcattacctgtacgcatcgatgaaaataattggttagatattacggctgttatggggatacaaacagaagaatatgcc |
| | | tcttttaagatacaacatgccgactgtggtcatagtgtagatagtttacttcaagcttatagaaatattaaattttcatttgcaaa |
| | | atgggctgaataccaaaattgtgtaccactattgggaagtacacgcgaattactgagaatagcacggtgggatataatgtacgaat |
| | | ttcgtgggcttttctcattcggttgccaggaacaggttactgcctacccggctaaaaatcgtattaacttcgattatcagggtaaa |
| | | accatcggctatagtgacttctggcaagcaataccattatcaatttatcctaaggatatacgctcacctgttgctacttacactgc |
| | | ttatgataaggaccttgcctgtaactggaaaaatcatagcgtactgaaaaagcctaatatcatgttatgtgattgtaaggtactaa |
| | | agagagaaaatagttacagtccttttgaaatatcagatattcgttttcactttgaatctgagccgttatagtaaggattattttgc |
| | | gataattaatcaacggggagctggtcaaagtgcctgctcccatattgactaatatacaaatgtgtttgttaagacctttccaaagg |
| | | tagggggaattatgaatttccgctcctcgctcatagccgcctgccagatttaaccccaccctaccacagggccccctcaagccaag |
| | | ccgccgccaatacaattttcccccacaccaaaacgcctccctccctagagcacgtactcacaacgccga (SEQ ID NO: 45) |
| 49 | pLG051 | gggatttccaccacctcccaccgaccatctaagactttatgccactgtccctaggactgctatgtactaggagcggatgttaaact |
| | | cagactcgtttcagctacattgcgttttgaataatattccatcataataactctttgaaaaatgtgatcttttcatttataacact |
| | | gatgacttgcttatctcattgggatatcggaggagaatacttaactatgacaagcccgattattatgacactggctatattatata |
| | | gattgatattaaaatgtaggattaggttcttgccaaggtgtcaagatttacagataggtttaaaaccatataaatatgttttacgg |
| | | tgagatacaatacatattgtaaggcataaacgcttggtaaaattttaattattggaagaagctaatcatggaacccatatcaatta |
| | | cagtggcaacttatgtagcaactaaacttattgatcaattcatctctcaagaaggatatggttgtattaagaaagcattattcccc |
| | | caaaaaagatatgtggatagattatatcaactaattgaagagacggcaattgagtttgaagaaacatatccagtagaaagtggagc |
| | | aataccattttatcattccgaaccattgtttgagatgttgaatgagcacatcttttttaaagagttccctgacaaagagatattat |
| | | tagacaagttcaaagaatatccaagtatcactcccccaactcaacaacaactcagccttttttatgagatgttatcattaaaaatc |
| | | aataattgttcgaagttaaaaaagctacatatcgaagaaacgtataaagaaaaaatattcgatattaatgaagagctcattcaagt |
| | | caaacttattttacggtctatagatgagaaactaacttttcacttaagtgatgattggttaaatgaaaaaaatagtcaagcaatag |
| | | ctgacttgggaggtcgatacacacccgaactcaacgtaaagctagaaatagcagagatatttgatggcctcggtagaactaatgat |
| | | ttttctaaaatattttattcgcatatagatagctttctggtcgctggaaagaaattacatagttgcgatgtaatttcctcagaatt |
| | | atttgaaataaaccagtccttaaaagaaatttctgatatatatcaggagattaatttttctaaattagatgaaatccctataaata |
| | | aatttaataactatgtttctagctgccagacagctattggcggagcggtatcaatattgtgggaactccgagaaaagtcagagcaa |
| | | gtaggtgaaaccaagcattacagtgataagtattcatctactctgcgaatgcttcgggaatttgactatgcgtgcaatgaattacg |
| | | tatattcattaattcaacaacagtgaagttggctaacaacccattcttacttctcgaaggaaaagcaggaattggtaagtctcatt |
| | | tactggctgatgtgattaaaaatcgaattgcttctgggtatccttcactactcatactagggcaacaacttacttcagatgaatct |
| | | ccatggtcacaaatcttcaagagattacagcttaaaatcacttctcgtgaattcctagaaaaactgaatttatatggcaaaaaaac |
| | | aggaaaaagagtcttagtttttattgatgctattaatgaaggtaatggaaataaattctggaatgacaatattaacagttttgtcg |
| | | atgaaatcagatgctttgaatggcttggtctgataatgtcagtcagaacaacatatagaaatgtaacaatttcacatgagaatgtt |
| | | gtgcgaaataattttgaaattcatgaacatattggattccagaacgttgagttggaagcggttagtctattttatgattattacaa |
| | | tattgagaggccttcatctcctaaccttaatccagagtttaaaaatcctctatttcttaagttattgtgtgaaggcattaagaaaa |
| | | atggtttaaccaaagtgcctgttggatttaatgggatttcaaatatttttaactttttagttgaaggggtaaataaatcattagca |
| | | tcgccaaaaaaatatgcattcgatcccagttttcctcttgttaaagatgctctcaatgaaatcataaaattcaaattagagattgg |
| | | tcgtaatagtatttcacttaaagatgctcactcagtggttcaatctgtagttaatgattatgttgctgataaaaccttcctcagcg |
| | | ccttgattgacgaaggattattgactaaaggcatagtgagaaatgatgataattctactgaggaagtagtttatgtggcttttgaa |
| | | aggtttgatgatcatttaactgttaattttttattaaatgatgttgaaaatatcgaaagtgaatttaagcctgatggtcgtctgaa |
| | | aaaatattttcatgatgaatgtgatttttatataaaatcgggaatagtagaggcgttgtctattcaattgccagaaaggtatgaaa |
| | | aagagctttatgaatttctgccggagttcagcaataatcttaaattactagaagcctttattgatagcttgatatggcgcgatatt |
| | | aaggctattgatttcgaaaaaattagacctttcatcaatgaacatgtttttaaatttaaagatagttttgatcatttcctcgaggc |
| | | agtgatctctatttcaggtttagttggccatccctttaatgctaatttcttgcatgattggctaaaagattattctttggcaaatc |
| | | gagattcgttttggactacagaacttaaatataaatatagtgaagactcagcatttaggcatctaatcgattgggcatgggccaga |
| | | acagataaaagctttgtttcggatgagtcaatcgagctagttgcaactagtttatgctggtttttaacttctagtaaccgagaact |
| | | tcgagattgctcaactaaggctttagtgagtttactcgagccaagaattcctgtattgagaaaaataattgataagttttatggtg |
| | | taaatgatccttacgtttgggaaagaatatttgcagttgcattaggctgtacattgcgaactgataatattaaagaactaaaatat |
| | | ttagccgaaactgtttaccaaaaggtattttgttctaagtatgtgtatccaaatatattacttagagattatgctagagagattat |
| | | tgaatttgctaatcatcttggattggaacttgaaagcattgaattatccaagactagaccaccctacaacagcatttggcctgaca |
| | | agattccttcaaaagaggaactagagtccctttatgataaagaaccttatcgggaactctggagctctattatggaagatggtgac |
| | | ttttcacgatatactattggaacaaattataatcattctgattggtctggttgcaagtttaatgaaacccctgttgaccgtaagca |
| | | agtttttaaaactttcaaatgtaaactaactgatcaacaaaaagacttgtatgatgccacagatcctttcatttatgatgataaat |
| | | gcgaaggaattaaatttggtcgtgtggtcggtagaaaagcacaggaagaaataaaggcgagcaagaaattatttaagaattcattg |
| | | tcatacgatctgttaagtgagtttgaaaatgaaatagagccatacctggatcataataataatctgctggaaactgataaacactt |
| | | tgatcttcgactagctcaacaatttatattcaatcgtgttatagagcttggttgggatccggagaagcatggtaattttgaccaac |
| | | aaataggaactggacgtggacgtagagaggcattccaagaacggattggtaaaaaataccaatggattgcttattatgaatacatg |
| | | gcaaggctagccgataattttactcgttttgaaggttatggtgacgaacgaaaggaaaatccataccaagggccatgggagcctta |
| | | cgtaagagatatagatcccactatcttacttaaagaaactggaacgaaaaaaataagcaataaagaaatgtggtggcttaatgatg |
| | | aagtgtttgattggacttgctctaatgaagactgggttaaaagttctactactataactaattcatatgcttttattgaagttaaa |
| | | gatgataatggtgatgaatggatagtattagaaagtcatccatcatggaaagaaccaaaaattattggaaacgatgattgggggca |
| | | cccacgaaaagaggtttggtatcagatcagaagttatatcgttaaagttgaagaatttgaaaattttagatgttgggcaatagctc |
| | | aagactttatgggcaggtggatgccggaatgtactgatagataccaattatttaatagggagtactattggtccgaagcatttaag |
| | | tcttttaaatcagattattatggtggatctgactggacttcggtaacagaccgggagtctggagctaagatagctgatgttagtgt |
| | | cacttcgattaattatttgtgggaagaggagttcgacaaatcaaaaatagaaactttgaattttttgaagcctagtaacttaatct |
| | | ttgaaaagatgggattaaaaagtggggaagtagagggtagcttcaatgatgaaaatggaactatggtttgctttgcagctgaagct |
| | | gtatatgcttcaaagccgcatctacttgttaaaaaagaaccatttttaacaatgttaagggacaatggttttgaaatcgtttggac |
| | | attattaggtgaaaagggcgttatagggggctcactcatatcaagtcatcattatggtcgacaggagtttagtggagcattttatt |
| | | atgaagacagtcagctaacaggaagtcataaaactagctttacgagataaaaatgaatctcagagctgaatatataagtagtatta |
| | | gaaaccgggttatacttaagaaatcaatcttaagtgtggcagtcgaatggtagctaatatgctagcggcgctaatgcctgtttgtt |
| | | gctcataacaggcattcactttagttatggcagaaaagtatacatgctgggttgggaaagtgtgaaagaaaggaagattgctgcgc |
| | | cgtttgtcgtcacgtttatcttcattggctatgca (SEQ ID NO: 46) |
| 50 | pLG052 | aaatctctttcgcgtcaatagtggtaatatttttttatcattgtcctctttctactgacatactgattgtccgacagtggagccag |
| | | tcgaaattgttgacagctagtcggggctcgtctggtctttctagcagtaagaaacgtattaatattggatcgccactagtttaaca |
| | | gatacctcagaattatttatagactgacaccaccccggcagacgatcctgccctataggaagctaagtggaaacttatccagtaac |
| | | agcttgtcgattttatcccagagggtgttcctcaggatgtatcgctgaaatcaaatccagcactaagaatgaggggtgagaaacca |
| | | tttccttggtgggtctttgaccatttctgttgaactaatgtttttgggttatcaaggatacaaattcaaggcagtgtttcactaaa |
| | | ccttacctcgcttcaataccaatacatttttaatgggtataatatgtgactgcttttgccgcattattgacaggaacaaggactgg |
| | | tgatgaatattgatttcagtttaattcgtagcgcccccaaaagccgtaacgatagctttgaagcactcgccgtacagttatttagg |
| | | aaaacctgtcgagtaccgacaaattcaacatttattagtctgcgtggagatggtggagacggtggcgttgaggcatatttccgctc |
| | | accggacggtgccgtattcggtgttcaggcaaaatactttttccagcttgcttccgcagagcttacacagattgatagttccctta |
| | | aagctgcgctaagcaaccatcccacactaaccgaatactggatttatataccgtttgacctgaccgggcgtgttgctgcgggaaag |
| | | cgaggaaaaagccaggcggaacgctttgaagaatggaaaagtaaagtcgaatcggaagcgtcagcgaaagggaagtcactttctat |
| | | tgtcctttgtaccgctgctgttatctgcaatcaattacttgagatagacccttacggagggatgcgcaggtattggtttgatgaca |
| | | cgttgctgacaacagctcaaattcaacaatgtctggaggacgccattgcttttgccgggccaagatatacttcaatgctggatgtg |
| | | gtgacgaatgctcatgtcggcctggatttctttggtgggactggtgacttttgcgagtggtacgaaacatcattaacaccaatcgt |
| | | tcgagagttccattcactgaatggatacggacgcaaatcgctggatatactcggcgaaacccgtgctacatctgccacggcattga |
| | | ttgaagaaataattgcctactgtgagagcatgagagataacaatgtcacggccacatcggttacagatctttccgtcgctctgtca |
| | | tccctattgacacttttcgctgatgcccgccatgctcaagaagataaattttatgaaaagcatggcaagcatagtgatacagaatc |
| | | gttccgacagttccacgcagagtatatgtgtgcatttcctgccggagatatggatgcggcgagaaaatgggaagagcaggcgcagc |
| | | aactgcaaaatttgctgacttctcaggtcattggtgccgcaacagcacattccttactgctggttgggccagcgggtatcggcaaa |
| | | acccacgcgattgtcagcgcagcattgcgtcgactggaacatggtggtttttcactggtcgtctttggagacgactttggcaaagc |
| | | agagccttgggaagtgctacgcagtaaaatagggctgggtgccgccatcgatcgttcgacattatttgaatgcatacaggcctgcg |
| | | ccgaacatactggcttaccttttgtcatttatatcgatgcattgaacgaaagcccgcgagaagtgcgctggaaggacaagcttccc |
| | | gaattgctcgctcaatgcaagtcttatccagacatcaaaatctgcgtttcaacccgagatacctatcgcaatcttgtggtcgattc |
| | | acgctttccagggtttgctttcgaacacatcggtttttcaggacatcaattcgaagcggtacaagctttcgcagcctactatgagc |
| | | tggatgcagagattacaccacttttttcacccgaactcggtaatcctttatttttacacttggcctgtaaaacgctaaagggcgaa |
| | | ggccgtgacagtctggatatttctttgccgggttttacctctctgtttcaaggacatctcaaacattgcgatgttttaattcgaga |
| | | acgcctccactacgcaaaccctcgtaatctggtaagggctgcaatgatggcactcgcgaaaaccctgacacatgagttgccgcaga |
| | | accgaacgtgggaaacctgttgcgaagcactgagcaaaatagtgggaactgagaccacacctgaatcctttttaaatgcattggca |
| | | catgaaggcctcattatcctttctgttgtagatgaggataccttcctgatccgtctgggttatcaacgctacggtgacatactccg |
| | | tgctatcagccttgtggaaactcttgattcggatacagtaaaactagcggagaaaattgcagcgttaacagaagaagatgctggat |
| | | tgctggaagctcttgccgccgtgctgccagagaaaactgctcttgaaattactgctgaagaagtaggattaccatccgaacaagcc |
| | | cataagctgttcatccagtcattggtttggcgctcccgacaaagtgtagtggaagaaattgatgaacacatccatgcagcactgca |
| | | tacacctggattatgggagtcggtttatgaagcgctgttttcacttagtctggttcctgaccatcgtctaaacgcaactaactggc |
| | | tggggccatttttacggcagtcatccttagctgaacgtgacacctacttgtcattagctgcgctgggatcatttgataataagact |
| | | gctgtctattcactcatccatgcagcactatttgctgacataacccattggcctgctgaaagccggaggctggccagtctaacact |
| | | tgcctggctcacttcgtgtgctgaccgccgaatcagggatttatcctcaaaagggctaagcagaatcctggcaaactacccggaga |
| | | actgccaaacagtaatcagtgaatttgcatattgtgatgatgattacgtattagagcgtattagccttgctatctacagtgcatgc |
| | | ttattgtcataccaacgcagaaatgcgtttatgccagcgctccctggtctattaagcattgcgtcagatagcaagaatattctgct |
| | | ccgggatacggttcagctattagtaaacttgttgaaaacaggagaatttcccacagccgtaacaagccaattacagcattaccaga |
| | | caaacgtatcattaccatcacgatggcctgtactggcggatgtcaaacccctcctagatctggaacatttaccatcaaacatggtg |
| | | ctctggggagaatccatggccccggatttctggcgttatcaggtggaatcgaagatttccggctttgacttggagagcgccaatat |
| | | cagccatgaaaacattgcctgttggttaatgcgagaagcacttaatttaggatatcccggttataaccactgcgcgctcaattatg |
| | | atcgccatatcgggagtcagtatggctcgggacggggtagaaaagggtatgctgaccgactcggtaaaaaatattactggatcgcc |
| | | ttacatcgactactgggcattctggccagtaatgttcccgcactggaagacccatattccgactacgaacctacaagtgatcttct |
| | | atggtcagtcgacgtccgtaaagttgacctgaccgatgtacgcgatatcaccgcagaaggtgtctatccagtactgatggaggaaa |
| | | caaattatgcattccctgaccacaattcagatatcaaaggttgggttaggaccgatgattttccaccttatgaagcttgtcttatt |
| | | cgaactgacgaggaaggagagcagtgggtagcgctttcacatagctattgggatgacgataaagcgccgaatgaaaatagctggga |
| | | ttccccgtacttgggagtgcgtgcttcctactcaagcgcactcataaatgaaagcatccagaactttaaacagaaaagatcacgcg |
| | | atattttccaatataatcagggaagtagttgttatcgcggttatcttgctgaatatcctgacagcccggtatacaaacaacttctt |
| | | aatagtgatgaagatagtgaagcgtttaattttacagaagtcagtttactgcgcggaaacgaatgggaatacgactactcatatac |
| | | catgcccgagcgccaggataacctcattgcgccatgcctgggaattattcaaaaactcgaacttttatgggattgtcaaagcggtt |
| | | gggttgatcattctggcaaacttatcgccttccatcaaaaaggtgtaaaacaacgcggacttttcatccatcgttcggcattgaac |
| | | gcctatctgtccataacaggtgaagagcttatacatcgccgttttgctaacagaggatattttgatttagctggtcgtaatagcac |
| | | gcaaatagacctgaaaacttggatccagtaccgggcagacaaggcaccggtagttttacgagaagaggaactgccgtttaactgct |
| | | gacaacgatacttattaagtaatcaactggctgccttggcatcgaatgccagaagagccatttcgcactaccaatttaagtagact |
| | | gaaggaatacttggtacaagcaaacgcacgccatatcggatagaggggact (SEQ ID NO: 47) |
| 51 | pLG053 | gcgcagctgacaaagattgaccgtgagcgctctgatggagaaagacgatagttgctgagtacgatatcgagggtacatttctctgt |
| | | gtaggggtagttatttacaaaaaaataggagaataattaaatggtcaaaccaaactgggataactttaaagctaaatttagtgaga |
| | | atcctcaaggtaattttgagtggttttgctacttgttgttctgtcaagaattcaaaatgcccgcaggtatatttagatataagaat |
| | | caatctggtatcgaaactaatccaataaccaaagataatgaaattatcggttggcaatctaaattctatgacacaaaattgtcgga |
| | | taacaaagctgatcttatagaaatgattgagaaaagcaaaaaggcttatccaggattaagtaaaatcattttctatactaatcaag |
| | | agtgggggcaggggagaaagtcccatgaacctgaaggcgataagaacgctgataattatttggaaactgtcggaaatagtaacgat |
| | | cccaaaataaaaattgaagttgatcagaaagcatatgagtcgggtatcgaaatagtatggagagttgctagtttttttgaatcacc |
| | | gtttgtaatagttgagaatgaaaagattgctaaacatttcttctcccttaatgaaagcatctttgatttattagaagaaaagcgca |
| | | agcacacagaaaatgttttatatgaaattcaaaccaatatagagttcaaagacagaagtattgaaattgacagacgacattgcata |
| | | gaacttctacatgagaatctagttcagaaaaaaattgtcatcgtcagcggagaaggtggggttggaaaaacagcagttatcaaaaa |
| | | aatttatgaagcagaaaaacaatacactcctttctatgtctttaaggctagcgagtttaaaaaggacagcattaatgagttattcg |
| | | gtgcgcatggcttagacgatttctctaatgctcatcaagacgaattacgtaaagtcatagtcgtagattctgctgaaaagctttta |
| | | gaactgaccaatatcgatccttttaaagaattcctgactgttttaataaaggataaatggcaggttgttttcacaacccgtaacaa |
| | | ttacttggcagatctgaactatgctttcatagatatttataagataactcctggaaacttagtaataaagaaccttgaacgcggcg |
| | | agctaatagagttatctgataacaatggatttagccttcctcaagatgttcgattattagaactaatcaaaaatccattttatcta |
| | | agtgaatatttgaggttctataccggtgaaagcatcgattatgtgagcttcaaagaaaagctatggaataagattatcgtcaaaaa |
| | | taaaccttctcgggagcagtgtttcttagcgactgcttttcagcgggctagtgagggccaattttttgtctccccggcatgtgata |
| | | ctggaattttagatgagttagttaaagacggaattgtcggctatgaagctgctggttacttcattacacatgatatatacgaggaa |
| | | tgggcattagaaaagaaaatttctgtcgattatatccgtaaagcgaacaataacgagttcttcgaaaaaataggagaatcacttcc |
| | | tgttcgccgtagttttcggaattggatatctgaacgattgcttttagatgaccagtccataaagccttttatcgcagaaatagtct |
| | | gtggagaaggaatatcaaatttttggaaagacgagttatgggtagctgtccttctttccgacaattcaagcatattttttaattac |
| | | tttaaaagatatttacttagtagtgaccagaatctattaaaaagacttactttcttattgaggcttgcttgcaaggacgttgatta |
| | | cgatctgcttaaacagttaggtgtaagtaattcagatctgctttccattaaatatgttcttactaagcctaagggaactggttggc |
| | | agagtgtgatccaatttatctatgaaaatttagatgaaatagggatcagaaatattaattttatacttcctgtgattcaggagtgg |
| | | aatcaaagaaacaaagtgggtgaaacgactcgattatctagtttgatagctctaaaatattatcaatggactatagatgaggatgt |
| | | ctatttatccggaagggataatgagaaaaatattctgcatacgattcttcatggggcggccatgattaaacctgaaatggaagagg |
| | | ttttagttaaggttcttaaaaataggtggaaagagcatggtaccccatatttcgaccttatgaccttaatccttactgacttagat |
| | | tcatatccggtttgggcatctctcccggaatatgttctacaattggcagatctgttctggtatcggccacttaaagaaacaggcga |
| | | acgttatcacagtatggatattgaagatgagttcggtctatttaggtctcatcacgactattatccagaaagtccatatcagactc |
| | | ctatatattggttactacaatcacagttcaaaaaaacaatagactttattcttgattttacgaacaagacaacgatatgttttgcc |
| | | cactcccattttgctaaaaacgaaattgaagaagtagatgtctttattgaagaaggaaagtttataaagcaatatatatgcaatcg |
| | | tctgtggtgctcataccgaggaacacaggtctctacctacttactttcatcaattcatatggcattggaaaagttttttcttgaga |
| | | attttaaaaatgcagactcgaaagtgttggaaagttggcttcttttcttgttaagaaataccaagtcagcttctatttctgcagta |
| | | gttacgagtattgtacttgcattccctgagaagacattcaatgtagctaaagtactattccaaacaaaggacttcttccgttttga |
| | | tatgaatcgaatggttctagacagaacacataaaagttcattaatctccctcagggatggctttggcggtacagattacagaaact |
| | | ctttgcacgaagaagatagaattaaagcttgcgatgatgtgcatagaaatacttatcttgaaaatcttgccttgcattatcaaatt |
| | | ttcaggagtgaaaatgtaacggagaaagatgccattgaaaggcaacaagtgctctgggatattttcgacaaatactataatcagct |
| | | tccagatgaagctcaagaaactgaagccgataagacgtggaggctctgcttggcaagaatggatcggcgaaagatgaaaataacta |
| | | ccaaggagaaagatgaagggattgagatatcattcaatcctgagattgaccctaaactaaagcaatatagtgaggaagcaataaag |
| | | aaaaactccgagcatatgaagtatgtaacgctgaaactatgggcaagctataaaagagaaaaggatgaacgttataagaattatgg |
| | | aatgtatgaggacaatccgcaaattgctttacaagagaccaaagaaataataaaaaagcttaatgaggaagggggtgaagatttca |
| | | gactattaaatggtaatataccagcagacgtttgttctgtattactgttagattattttaatcagttgaataatgaagagagagaa |
| | | tactgtaaagatattgttctagcgtattctaaacttccgttgaaggaaggctataattatcaggtacaagatggaacaacctcggc |
| | | aatttcagccttacccgtgatttatcataattatccaatggaaagggagactataaaaacaatattacttttgacactgtttaatg |
| | | accactctattggaatggcaggtgggcgctactcagtatttcctagtatggtgattcataaattatggctagactattttgatgat |
| | | atgcagtccctattgtttggttttttgattttaaagccaaaatatgtaatcctttcaagaaaaatcattcatgaaagttatcgtca |
| | | agtagactatgacattaaaaaaataaatattaataaggtgtttttaaataactataagcattgcatatcaaatgtcatcgataata |
| | | aaatatctatagatgatttgggaagtatggataaagttgatctacatattttgaacacagctttccaattaattccagttgatact |
| | | gttaatattgaacataagaaattggtttccttaattgttaaaagattttctacaagcctattgtcaagtgttcgagaagatagagt |
| | | tgattacgctcttcggcagtctttcttggaaagatttgcctactttacgcttcatgcgcccgtgagcgatattcccgattatataa |
| | | aaccttttcttgatggtttcaacggttcagagcctatttcagagttatttaaaaaatttattctcgtcgaagatagattaaatact |
| | | tacgccaaattttggaaggtttgggatttgttttttgataaagtggttactttgtgcaaggatggagataggtattggtatgtaga |
| | | taaaattataaaaagttacctttttgctgaatctccatggaaagaaaactctaatggttggcacacatttaaagatagcaatagtc |
| | | aattcttttgcgatgtatctaggactatgggccattgcccttcaactttatattctcttgccaaatctttgaataacattgccagt |
| | | tgctatcttaatcaaggtataacttggctttcagaaatattgtcggttaataaaaagctatgggaaaagaaattggaaaatgatac |
| | | tgtttattatttggaatgtttggttaggcggtatattaacaatgagcgtgagcgaattagacgaaccaaacagttgaaacaagagg |
| | | tcttagtaatattggattttttggtagagaaaggatcggttgttggttatatgtcacgggaaaatattctgtgatgtagttgaaaa |
| | | taataattttaatgagagcttttccaatttaggctccagggattggagcctttttattatcg (SEQ ID NO: 48) |
| 52 | pLG054 | accttcttcgctaactgatggctaatgaggccgtaataaaacttaccttacctgtaaatacttttactactcattcagatcagaat |
| | | gaagaggtttattttatttcattgaaaattaataaataaaaatattggcacggtatgtgcttatacagaatgccattttactaaca |
| | | aggaatttaccgatgtcggaattaaaaaaatttcaggtacaaacagcacgtgcattgccggtgattgtgttggcggataccagtgg |
| | | gagtatgtcaacagatggcaagattgatgcacttaatctggggctcagggaaatgcttgatagttttaaacaagagagccgcctgc |
| | | gcgctgaaattcaggtcagcgttattacgtttggtggtcaccaggctgaagttagcttgccattgacgcctgctcaccagttgcaa |
| | | agtattacctccctggaggcaaatggcatgactccactgggtggcgcactatcgctggcctgcgagattattgaaaatccaacgcg |
| | | aaaatttcagccgattatcgtgcttatctccgatggctaccctaacgacgactgggaagccccttttgctcgcctgattcacggtg |
| | | aacttactgccaaggcctcccgttttgccatggctatcggtgcagatgccgatgaatcaatgctcaacgaatttgcaaatgatcct |
| | | gaggctcctctcttccacgcagaaaacgcgcgtgacattcgccgttttttcagagcggtaagcatgagcgtcagcgcacgaagccg |
| | | ttccgcaaccccgaatcagtctacaccgttgcagatcccgagtgctgatgatcaggactgggagttctgatgcgcctgtacgcttc |
| | | tggcacctcggtacgtggtcccgcacaccaacaggatgatgaacccaatcaggatgctgtagggatttacggtctgcgtggtggct |
| | | ggtgtattgccgttgctgacgggttgggtagccgatcaaaaagtcatttgggttcccgtaaggcagtcaatctgctgcggcagatc |
| | | atgcgcggtgcggagatgctggtcgctgccgaagtgactccagcgttacgtgaagcttggctaaaccactttggtactgactatca |
| | | cgattacgaaactacctgtttgtgggcctgtgtcgaggcgtcgggccatggcgtgatcggacaggtaggcgatggcctgctgctgg |
| | | tcagaagtgctggggtgttcaacgtaatgagcacaccacgacggggttacagcaatcacactgagactctggcacagcgtgcacat |
| | | ttagatagttgcagtgccagagtggcattaacccaacccggagatggcgtactgatgatgaccgacggtatcgctgatgaccttat |
| | | cccggatcagctggagtcattctttaatgctatctaccaacggatacggcaatgcagcaagcgtcgtacacgtcgctggttaacac |
| | | aggaacttaacggctggtcgactccaaatcatggtgacgacaagagcctcgctggaattttcaggatggactgaccacatgacatc |
| | | aatagtaaaaacgcaaccaaaacgcgtggtgaaggataccaggggatcaagttacgagctgacagaggtaattaaccgtggtggac |
| | | aaggcattgtttaccggacgacctatccgcaaaccctggtgaaaggttttactaatcaggacccacaggaacgccagcgctggcgc |
| | | aaccatattacatggctgctcagccaggatcttagcgacctcaaacttgcacgtccattaatacttctggcggagcctcgctttgg |
| | | ttacgtaatggagctgatggatggcctggttccattggatagcctgttgaacagctttataaacgcaggggaggagtctctggcgg |
| | | attatctgcgtcagggaggactccgtcggcggattcgtatcctttgccagctggcacgcacactcaatcagcttcacgcacgcggc |
| | | atgttgtatggtgatctctcccccagcaatatttttgtttcagacgatccaagacacgcggagacctggcttatcgactgcgataa |
| | | catcagcctgacagcccatcacaatctgactctgcataccgtggactatggtgctcccgaagtggtcaggggagaatcgttactgt |
| | | ccagcctgaccgatgtatggagcttcgccgtcattgcctggcaactgctgactcataaccatccgtttaaaggggaactggtcagt |
| | | aatggtcctcctgagatggaagaagctgccatgcgcggtgaatacccgtggatcaatgacgcacaggatgacgcgaatcactgctt |
| | | cgtcaatctgccaccggagctgattgcacatagtgcactgccaactctcttcgctcgctgctttgaacagggaaggtttgaacctc |
| | | atgagcgtccgggtatggctgaatggcttgaggcgctgagtgctgtggatgagcgtctgtttacctgtgacagctgtgggggaagc |
| | | acgctcctggcagaggaagcagaaagcgcgaacgatgccgtttgcttttactgtgacagtcccgccgaccgcctcctggtccggtt |
| | | tagtgaatatgtgactgagcaacaagacggctcgaatccagacaccaaaaccttgattgccacagggcgaaatgtatggctgcagc |
| | | caggtcaccgtgttgagttaaagcgcctgttgccaagttttatctatgaccactggccatcagatcatctgcagattgattacacc |
| | | gcccgcgggattgggatccatccgttgcttggcggagagctatacctacaacgcggtgaaactatcaaaccactgcgggggtttca |
| | | gggactcaaaaacgagctgcgcggaacaggtggggagccttggcagatccatatcggcgatcctggccagtcgcatgtaatctggc |
| | | agttcacgtggtgacaatatatgaaaattaacgaatttccactgatgtccaaagatattctgctgctggaaacggataaaggaacc |
| | | accgggttccggccaaagcaagctatcacctttcaggcgtatggtgagaattggctggcggtacagggggatcattgcgtaagtgt |
| | | ccagtgctcccctggtgatcacgaactctttagccgtctggtgatgagggatcaggttcgttggttgctgaccagtaaagcggaaa |
| | | aacagttgcgggttcaatattgcacgcctgttgaagtcacaccaatgcagctcgagttgggaattgatgagcgaattgcggaagac |
| | | cttttcgcgaaaaaacagatcaataacaacgatattgagcttgcctgccgctggtttgaagagacttttattgtccatagcgagtc |
| | | agaaagtgactggttaacggttggccgttttagcaatcatgcagccaaaggtggttttcagctattgggaaacggctggcgtgcgg |
| | | atgttgagcgcaacccggaccacggctttcttatcagacgtattactggtcatttaagccatgatacaggcttctcgttgctggtt |
| | | ggacacttcgccttccgggatatgtcagttgctgcggtgctgaatagtgcaacccagcaggcaatgctcgatgccgcactgcgaga |
| | | cagtgccagctaccttgagctctggaatctctacaacgataaagagtggcagagcgagttgaaaaaggccgaaacgctgggtgttc |
| | | tgcgctttgttgcgtgcgagggcaccgaagctggccgggaaaatgtctggcatctgactccccgaactcctgaagaatacagagaa |
| | | tttcgccagcgctggcgcgcgctcgatctgcccgcaggcactcaggttgacctgggcgctgaaactcccgactgggcagaagaact |
| | | cagtaccgaagaggatacggtactgaaaacgccgcgcgggaagatcgagttcgctgatgaatatgtggtctttacttcagcctcga |
| | | atcgccgagacgtgcgccccgcaaagcctgaaggatggctctacctctcgttggcaggatatcgcacagtcggcaaacgtcgcctg |
| | | gcggcaaaacgtgccattgattccggtaaacgcatgccacagttgaagtggctgctggaaggggtcgttgttcctgctgctcggcg |
| | | tcgcaacatccaggggatgacaccctacgcccgcgaaatctttaagggtggcaaaccaacgggcaaccaggaactggctgtgttta |
| | | ccgctctgaacacacccgacattgctatcgtaattggcccgcccggaacagggaaaacccaggtgatcgctgcgctacagcgacgt |
| | | ctggcggaagaggcccaggaaaagaatattgctgctcaggttttaatcagcagttttcagcatgatgccgtcgataacgcgctgga |
| | | ccgcagtgacgttttcggtctgcctgcatcacgtgtgggcgggcgtcgtgcttcagtagaagacgagtcaccactggatccctggt |
| | | tgtctcgccacgccagtcatctgcaggagaaaattgctgaccagtatcaacgctacccggagttgaaaacaattgccgacctcact |
| | | tcccggcttgccctgcagcgattggcaaacgacctgcctcaacaacgggcagaggctttttcgcatatttatcaggacgtcaattc |
| | | cctggcagagaaagggctggtcacggactcccggcttgagatacgtctgcaggactatattaagcatctgaaacaggatggtgttg |
| | | ctgaggtcagtacggtgatgaatgtagcagtattgcgccgcattcgcgcgttacggaccactcagactgctttctcagatgatggt |
| | | gccgatcgtgcctgggatttgctgcgatggttgaagcggaatgttcctgacatcgacgctgagctgacctcggtattggaaatagc |
| | | tgccgatgccagagaagttcctgtggcactcgtcgagtgccagcaacagctgctggagcgttttctgcccgattatcgacctccgg |
| | | ccctcaaaaataagatcgatgatgaaggactggctctactgaatgacctcgacaagcatctttccgacttgatgcatcggcgtaag |
| | | cagggtgtggcatgggtgcttgaacaaatggccgatacgctggagatggaccgccgtgccgcacaggaggtggtggatgaatacgc |
| | | catggtggtgggagcgacctgccagcaggccgccgggcaacagatggccagcctcaagtcggtttcaggagtcaagagcagtgaca |
| | | ttgagttcgataccgtagtcgttgacgaggctgcacgcgccaaccctcttgacctgtttgtgcctatgtcgatggccacgcggaga |
| | | attattctggtcggcgacgaccgccagcttccgcatatgctggaaccggatattgaaggccagttacaggaggagcatcagcttac |
| | | ggcactgcaactggctgcctttcgttcaagtctttttgagcgcatgaggctaaagctactggacctgcaaaagaaagataatttac |
| | | agagggttgtgatgcttgataagcagttccgcatgcatccactgctgggagatttcatcagccagcagttttatgaaaaagaaggg |
| | | ctggggagagtggaaccaggccgtagcgcagaggaatttgtctttgacgaaggtttcctgagagcgctggggccactggcgtcggc |
| | | ctatcgtgacaaggtctgccagtggatcgacctgcccgcttctgctgggctggcagaaaaatcaggaaccagccgtatccgcacca |
| | | ttgaagcggagcgtattgctcaagaggtggcacagttactgaaagccggaggagaaaccctctctgttggggtaattactttctat |
| | | gccgcacaacgagaactgattatggaaaagttatccgaaatcaggctggaaggcgtgccactgatggaaaaacgtaacggaaccta |
| | | tgaaccgcatgaaaactttcgctgggtgcgcaagtaccgtgctgacggttcgttcagccaggaagagcggttacgagtaggttcgg |
| | | tggatgccttccagggtaaagagttcgatgttgtactgctatcctgcgtgcgcacctggcgtcagccgaggtcctcatctgccgcc |
| | | gatgatgcagctgccagggaacaaatgcttaatgaactgttcggtttcctgcgtctgcctaaccgcatgaacgtcgccatgagccg |
| | | acaacgacagatgctgctttgcttcggcgatgcagcactggccaccgctcccgaagccctggaagccgcgccagcactggcagcat |
| | | ttcataccttatgcggaggcgttcatggcactcttcgctgaaacaggtatttatattcaatctgccccacggccgcagggtgaagc |
| | | gcgcccgatactctggccagtcaggatacatagggtgctctacccggaaagctatcaggctcagatcaatgtcttccaacgcgcaa |
| | | ttctcggattggtacgagcgcgcgtcgtacgtccgaccgaactggcagaactgaccggtctgcaccctaaacttattacgcttatc |
| | | ctggcacaaagcgtcagtaatggctggcttgagtccggtgaagataccctcacttcagcgggtcagcggttgctggatgatgagga |
| | | tgacggtattggcaaacaaaaatcaggctatgtattgcaggatgctgtaagcggaaagttctggccgcgtctggtcagcacattga |
| | | agcaaatcgaaccggtcaatcctctggataaatatccgcaatttatactgaccaggaaaacaggagcgacactgcgacctttcctg |
| | | atgaatgccagccgatcgccactgccgcctctggaacgcaaagaactgaagcgtgcctggcgtgactatcgtgacgactatcgtgc |
| | | cagtcagcaactgggcgtcagccgtttgccgccacacattaacctgcacggtctgcagcagctagaggaaccaccgcagtgcgcac |
| | | gaatactggtgtggatcaccactgatcgagagagtggacagctatggagtgccgcggacccatttgctctgcgcagtaacgcatgg |
| | | tggctggacctgccttcaatcgtggaaagtgactcccggttgcaaaagatactggaaccgctggttgtggtgccacgcgccgcaga |
| | | acaaacctaccagcagtggcttgaggctatcgcgcacgaaactgattttaagatgatgagtcaatacccttgggccgaacgtttac |
| | | cggatgtgaaacgttatttggtggcgctattggtacatagagggaggatcgagcagggtgataacggtcaaagtgagctggatgcc |
| | | gcactgaacgagtgccagaagctgctggaggttgttatgcagtggctgattcgtcgtcatccagccaacgcggaattattacccaa |
| | | gggccgcctggataaaattaatacggccaacttgctcaaggatatgaaaataccagcatttaccccatcagttattgatggcctat |
| | | ctggccagataatacgtcaggtgcgctacgcatgtagcaacccatccggctcattgaaggcactactttttgcagcggctgtcggt |
| | | gcgaaccaggatccacagcacccattttggtcactggatgactcagcgttacaactgccaatgctgctgcaactggcggatcgtcg |
| | | caacaagagtagtcatggacagagtaaatatcttgataagccggtacaggaactcactcagcagatggttgaggaaagtatcagtt |
| | | atgcattgagttttaccgaacgttttaaggaatggatgtaatgtcaaaacgagcacaacagaagtatacctcacctattcccaagc |
| | | agagaaatggctctgctgcggcatctgccatcaccacacttcagaggtctgcaatgacaaccgagtcgcagattattgccgcagcc |
| | | catcacacagctcagagtgaaaagcttccaaaagatatcgattttgatgtgacatggctggaacgtatcagtcaacgtcttcagca |
| | | ggaaggagatgatcaatttgtctcctggcttcagacatttactcttttctgccagaaactggcgcaaagggatgaagagacgcaag |
| | | cagcagcacagcgtattcaacagctggagctgacgctggaggagcaaagcgaaaagttagaacaggaccgtgttgaacatgacatt |
| | | caagctcgggaactggcggaaaagaaagccgggatcgtgagcaaagaacgagagctgaatgaacgtgagctcaacgccaaagcggg |
| | | cttcagcgagcagaatgcagcatcgctgcgaaacctgacccagaggcagcagttactcgaccagcagcatcaggaggatattcaac |
| | | agctcatcacacaaaagcaggggttaatgcgggaaatatcgcaggccattgtccagttgacccagttacaaatccagcaaagcgac |
| | | gcggaggcacagcgcagcttgtcactggaccagcgcgaagaagacatcatcaggaaagaggaggatctgaagcgcgccagccgtcg |
| | | tctggaacgagacgagcggtctgtagaggcggagagacaggcgctgaacgaatgtttggctgaagcaatgcaaacagaacgccttg |
| | | agtttgaaaagaagctggatcagaaagagcgtcagttcgacaaagctcaggaacgggtgcaaaacctcagtgaacgcctcatggaa |
| | | tgggaggaacttgatcaggcgctcaatggccaatccgcttcgcaaatgctgaatgagctggataagttacgcgatgaaaaccgcga |
| | | acttaaaagtcagttcgcgcacactaacctagcagagctggagcgcgagaacaaatctctggccaacagcaaaagcgctcttaaaa |
| | | atcagctggaaaatctgcttgcagagatggacaagctacaacgcgaggtggatcttcagcgagtggctgcgacccagcttgagaca |
| | | gtggcacgggagaagcggcttcttgagcagcagaaacatctgcttggtcaccagattgatgagattgaagctcgtattggcaagct |
| | | gaccgatgccagcaaaacccagacgccgttccctgccatgtcacaaatggacgagaagaatgggctcaacgcaaaacgtgatcatc |
| | | gagaggtcggtgacctgaaaaattttgccagtgagcttcagcagcgtattgctcaggcggaagagagcgtgcagctattctatcca |
| | | ctggaaagtatccagctgctgcttggtggtctggcgatgagccaactgcacctgttccaagggatcagcgggaccggaaaaaccag |
| | | cctcgccaaggcctttgcaaaagcgatggggggattttgtaccgatatttcggtgcaggctggctggcgtgaccgcgacgatcttc |
| | | taggccactataatgccttcgagcggcgctattacgagaaagactgccttcaggcactctaccgtgctcaaacaccgtactggcag |
| | | gacacctgtaatgtcattcttctcgatgagatgaatctttctcgaccggagcagtattttgctgagtttctctcggccctggagaa |
| | | gaacagccacgctgatcgaaaaattgcccttaccgaaacagctttactcaatgccccggaacggctcgttgaaggacgccatattc |
| | | tggtaccaggtaacctgtggtttattggcaccgccaaccatgatgaaaccacaaatgagctggccgacaaaacctacgatcgtgcc |
| | | catgtgatgacactaccgaagcacgacactcgctttcctgtcagggagatggagaaaaccagctattcgtggcggtcactgcatga |
| | | agcctttgctaaagcaaaaacgcaacatgcggaaacggtcaggaacatgctggagcaactgtccggtcatgaatttactcacctgc |
| | | tggaaacagattttggcatcggctggggcaaccgttttgacaagcaggcgatggatttcatcccggtgacgatggcctccggggca |
| | | gaagctgggcgcgcgctcgatcatctgctggcgacccgtattatgcgctcaggtaaggttaccgggcgctataatattggcttgga |
| | | atcggtcacacgactcaaagaagaacttgaatttttctggattcaggtcggtctgcaaggcgatccggttgaatctatggcattgc |
| | | tggaggcagatatccgccgtctgtcaggtgcgcgctgatgtggcacgatcgtttaactggtaggcaacatgcacatcttccgcaac |
| | | ggattgatcacgggcgttactcaatcgaggcttcccctctgacgctaaatggacatacaccgaattttttcggattgctggtcagc |
| | | gacggcggagcaaattgtcggctggacgatacgctgcataacttcattcagcctccgcccggccatgaagaggaaacccggctgct |
| | | ggaggaagccatcaccacgatcggtgccgcagttgatgatgacatcagtgtgctatcgccgctgatgccagcagctattgtcgata |
| | | atcaaagccttttgctacctttcgaacgtgcactgctggaggtgatacaaaaaggacatttacagcatatatcacagcggccgcgg |
| | | ctggatttacgttatgacgatgaggtggccgacgttgcccgcgtgcgtcgtctggcaaagggtgcactggtacatctggcgtcaca |
| | | ctccgaatgctggcagcgtcagacactcggcggcgtggtacccaagcagatactggcacagtttagcgaagatgatttcaatatct |
| | | acgagaatcgggtttatgcgcgattactggataagatcgaacgtcatttgtatcaccggctgcgcactttgagaagcctgcaatct |
| | | actcttgcccaagcactggacttctatcaatctcaggaggtgaattaccgcctgcgcaatgctatttgtcagttgtgggggatgac |
| | | ttacgatgaggatgcgactgatggcgcatctcggcagctcaacgccacattggcgacgctggagcaaattttccgcatcatttccg |
| | | gtctgcgacaaagcggcctctatctgcgggtaagtcgtactgcgcaagtgacaggtggagttcatatgacgaatattttaagtcac |
| | | gatcctcactatggtcatttgcctttactatgggcacagttggctgacggggctcagcccgaaaatttgcctcaacaacgcctcag |
| | | agtgaaccagagcctggcagctgcgtatagcagctatgccgggttggtgttacgccatgcgttgcagccctggttacacggtaaga |
| | | gtgaaggaagctgggctggtcgcactctgcgacttcgccagcaaggcatggaatggctgctgagctgtgattccaatgacagtgcc |
| | | agtgaagagacgctgttgtctctggtgccatttctgaaccaccagcaggtagcggtagacctaccggaaaatcggtatatcgcctg |
| | | gccttgcgtggggcatttacagcaggcattacctgataaagagggctggattcggctttcacctttagatatgtactgtgtagagc |
| | | gttttggcttactgatagataaaattcttagccgggaattattgcgaaactttgcccgtccggttatccgtattccccggtgcgta |
| | | ttaccacttgctacaaaactgtcttcactgacagttgatcaacagttaaatcagataacactgcatggggatctgactaaagctga |
| | | gctggaacaattaacctctcatttaatcaacaacaatgctagcacacaggcagaggaaattacgctgcgataccgggaatggcgag |
| | | cattgcaacagtgccctgtctgcgaccatacaaccgaactggtttatcaatatcccggtggatttaaaaccctctgtaaaaactgc |
| | | aataccgctcgttatttcagccagcatgaaaatgcacacttttttgaacaaaccagaacagtagaaagagaaagtaaaaccttcct |
| | | ggctcaggggcggagagtttttaactttcagttttagcagggtttttacgactcgctgcatttttaaagagttaagaataatgaaa |
| | | cttcagggcatcttttatatatcggtattacgcaaatcagtagtttcggttgcgcgttttgtatacataccggcaagtgtccaatc |
| | | acagtgaatagccaaaatcgccgggagcacgttcggtcagcctgcggacatggtttttatcacgt (SEQ ID NO: 49) |
| 53 | pLG055 | ggattcaccattatagtgacatgttcaagatgatgatatatctttgaaaagtgttctctttgcgaacggtatagaatttctagcgt |
| | | tacttttcataattacactttttagggttaggcaggcacaatctatgcgctgtcttagataactacatccatttttactggactac |
| | | caccaacaaaaatttagtggtgcaggagaaaacgtgaagtatcagatagtaggtggtgctggcctgcaccgcagcgaaaccaaaac |
| | | agttgatatgatggttaagcagttaccagatagttggtttggctatgctggcttagttgttactgatagccaagggtcgatggaaa |
| | | tcgatatgctaattattactgctgaccgtctgctattagtcgagcttaaagagtggaatggtaacatcacatttgaaggggggaag |
| | | tggctgcaaaatggtaagtcacgaggcaaaagtccctatcagatcaagcgtgagcatgcactgcgactaaaagatttgttgcagga |
| | | agagttatctcgtaagctgggttactttttgcatgttgaggctcatgtagtgctgtgtggcacagctggtcctgaaaacttgccat |
| | | taagtgagaggcgctatgttcatacccgtgatgaattcttgactataggtaacccaaaaaattacgaaaagctggtgcaacacact |
| | | aacttttttcatctttttgaagggggaaagcctcgaccaaattctgatgaggcattacctataattaagtccttctttgaaggacc |
| | | aaaagtcaggcctttgccactaaaagaaagcggttatcttgcgaacgataagccattctttagtcaccctcacatggtctacaacg |
| | | aattcagggctacccacaaagacaatagtcaacacagaggtctgctacggcagtggaactttgatgccttgggtgtagcaaacgca |
| | | atgcaaacattgtgggctgagatagctctgcgtgagactcgagtcggtcgcctagttcgtcatggcagcgcaactatgcaggatta |
| | | tatgttgcgtgctgtaagggaactatccgaggaggatataactgatgatgcccgtgagctgtatgagttacgccgtagttttagcc |
| | | gattagatgagattctagatagcgaagctgacggatggagtaaatctgagcgtattgatcgcgttcgtgcattattagctccattc |
| | | tcggaattacatagcttgggtatcagtcattgtgatattgacccgcacaatctatggtacgcaggggatcagaagagcattgtcgt |
| | | tactggctttggcgcagcctcactggagggacataatagcctagagtcattgcgtccgacattgcaaagtgctccatatattttgc |
| | | ccgaagatgcttttgaagaagcagttgagccctatcgcctagatgtattcatgttggctgtaattgcttatcgtatttgttttgca |
| | | ggtgaatcattactgactcctggacagatgcctgaatggagagctccattaactgatccttttagcggtattctaaatagctggtt |
| | | tgagcaagctcttaaccttgagccaagtaaacgctttccacgtgcggacataatgctcaatgagtttaatgcagctactaaggaac |
| | | atagccaagaatttgatgaagctaaccagatttatcaagaattaaagcaaaacaaattctttcgcgaagggatgaacagcgttggt |
| | | gtgttaattgagtttcctccacttcctgaacagttgtctatggtttactctgctcttgctgctattgctacgactggcagcatcag |
| | | ttatcactgtgaacaaggtgggaaagctctgcaggtaaaattgtgggatggtgttattttgacccctcaacaacctggtgttaacc |
| | | gccgtatccacgcttttaagcaacggatcgataagcttacgcatataaatctgccaactcctaaggtgcagtcctatggactatta |
| | | ggacaaggcggcttgtatgtagtgagcgagtatgtggatggcctaccgtggtcacagtttattgctgagaacgtgttagtacaatc |
| | | ccaacgttttacaattgcggaaaagttgatcaacaccattcatgcttttcatgaaaagcagttacctcatggagatctttgcccag |
| | | agaaactgctggtacaagtcggggagcagacagtaattactctgattggattgcttgaattcagtgatgaattaactgcagataat |
| | | cgctaccagccagagaatcccgaaagtactgatgcttttgggcgagattgctttgcagtatatcgtatggtggaggagctatttag |
| | | tgaagatatgccagtactggtgcaggctgagctagaacgcgcaaaacaaaccgttgacggtatacctatcgcgctcgatcctttgc |
| | | tgcagtcaattcgagcaccggaacaagctgagattaatcaagttgtggcgtctgagtcacaggataaggtaattcctgtttgctgg |
| | | ggcacagatgattggccgcaagaagtgaagcttctagaacaaaatgatgggatctattattttcaatgtaactggtcatctaaccc |
| | | acgctttgcgcatgaattgcgttgttacatcactggcctaggagagcggctattgatagacttagatcctgataatcgcactatta |
| | | atagaatagtgtatgaaaaaggattatcgatcgaagaaagtataaaggctggtaaatattcccaggctaaaattaatactcaactt |
| | | tcattacaacgtggctcacttaatcagcgtaatacttttattgaactactgtttaacctcgagccagtaattgatgccatcattga |
| | | gcgagctaatcctaatcaagagatggatgaagatgacttcgatagtagtgagtcaagcccaattgagttatggcaggcattatctg |
| | | atacagaagtagacctacgagatatagtcaacatcgactctactgactttcaggaatcaccgagtggttgcttactctacccatat |
| | | actacggaatccggtgctgacctcagctttgaacttgatgataagatcattgtttatattaaagataagcgtgaatcagtgcaatt |
| | | aggggaattgcagctaagtgagactacgccgagtctattggctattcgctttgattttgatgctgctcgtaagcgaattagtagcg |
| | | gcagccagctacaattggaatcgatccgtgacaaatcatcaagagagttgcgtcaaagagcccttcaacgggtaattgaaaacaaa |
| | | gcagagatccagcatctgccacagtattttgattaccaccagaaaccctgcatgcagcaaatgcaaccgcggccatccgcggagac |
| | | attacgcgcactttatgatcagcctggacaacgttttaatgaacagcagctaatggcatttcaacagttggtcgagtttggaccag |
| | | ttggagttctgcagggaccacctggaacaggtaaaacaacatttatttcaaaatttattcactatctgtatcaacattgcggtgtg |
| | | aataacattcttttggtcgggcaatcccatgcctctgttgataatgtagccatcaaggctcgagagctctgccatacgaaaggaat |
| | | ggaactggatacagtacgtattggtaatgaacttatgattgatgagggtatgctaagtgttgcaactaaagctcttcagcgacaga |
| | | ttcagcataaatttcaccgtgaatatgatctgcgagttagctccctaggaaagcgcctagggatggccccattattagtccaacag |
| | | ttatgtcagttacatcgtacgctgaatcccttgatggtgacatatggccaatatagccgtgagctggataaagtagaacaaataaa |
| | | gagtagtagtattagtcatcaagagcgactggctgaattattagaacaaagcaatcagcttaaactgcgaacacaagaaattatta |
| | | actcaatattcgatgacagcttgctgaaaactcttgtctatgatgaaaccttgataagacagttggctgagcaagttgccatacaa |
| | | tacaattataacaatccagagaaccttgaacgttttatgcagctattggaaatgagccaagagtggatggatgtattacgcggcgg |
| | | cgaggctggatttgatcgatttatgttcaaaagtaagcgattggtttgtggaactcttgttggtgttgggaatcgtcgactagaac |
| | | tagctgagtccagctttgattgggtaatagttgatgaggctggccgagcacaagctgctgaattgatggtagcgctgcaatcaggc |
| | | aagcgggtgctgttggtaggggatcataaacaattgccaccattctatcatcaacagcatcttaagttagcctctaagaaattaga |
| | | actcgggaaagggatcttttatgagtctgattttgaacgtgcttttaaagcaacaggcggcgtaacactcgatactcaatatcgaa |
| | | tggtagaaccaattggcgagttagtatcggagtgcttttacgctcaagatatcggtaaactgcattcatcgaggaaagtctcgcca |
| | | gattggtattccaagttaccaatcccttggaacaaaactgttacttggatcgatagttcgagccctaatgaagcaggtgcagaaga |
| | | acataagggtaatggtcgttactataatcaacgagaagtccggctactgctagaggctttgcagtcattgtcgagtgatggctgca |
| | | ttgcacagcttgagcaaactattaccacagaacagccatatcctattggtataatcacaatgtatcgtcagcaaaaagaggaaatt |
| | | gacaatgctatcagtcgggctgaatgggctgcatcgttacgtggtttgatcaagatcgataccgttgattcatatcagggccagga |
| | | aaacaagataattatcctcagtctggttcgcgataatcccaacaaactacaaggtttcctgcgcgacgcgccgcgaataaacgttg |
| | | ctatttcgcgagctcaagaaaggttattgattctgggagcaaggcgtatgtggtcaaagaccaataatgattcagcacttggaaac |
| | | gttcatgaatttattagtaaacaggttgcagtagatgaacccaactaccaaatcctgtgtggtcaaagtctgcttggagataacaa |
| | | ctaatgtcagaaccacgtctgggtaatctgattaccgttttactacctgcgcgtagttacaagatcaactgcgctttgaccactga |
| | | aaaactgatgcctggaattgaacagtttgcatgtcgcttgctgctgatttttgatcaactctatcccagcgagttacagaattact |
| | | ttggtctaactgatcgtgagcgagaggtattgcttgatgggttgctggctaacagactgatcaacattaatcctgatgggcatatt |
| | | gaggctagctcattcctacgtaagcatgcagctaataatggtgggaagccaagtttagttaaatatcaagaatgtacggaggaagt |
| | | tgcattcgatctactaactctttcgatatgtaaaccgcaaccaaatcgtcgttttacttctggactgccagagctattgccgcggc |
| | | atcagatcgggggagatgctgctgcggtaacagaggcttttagttcccagtttcggcaccatcttttgctcagccgcaacagcgag |
| | | tatgagcgtcaacggactaaattatataagataatgggctgtagttcgcatgagatggtgcagctcccaatagagatagaggttag |
| | | ctacggtgtttctgctgggagcattgagccgcagaaatttactcgttcctatgaatatttaggtaacacccggctgccgctttcaa |
| | | acgagctggaagctcatatcgcagattttttgggagaacataaactagatgaattcggtatcgactgtgaagatttctgtaaacta |
| | | gcaaatgataaagtgttgttacaatttgctaatggttataagttcaactattccggctggatagaggctcgtgaacaacgtaaaac |
| | | tggctacggtacttcattgactaccggcatgttaggggctgtttatttgccgcacaattctaagctgttcattagtatgttgcata |
| | | atgcattacgtgattatataggtaaaacagctccaaaagcgctgtggtatagcagtaaagtaccactgtggggagctaatggtagt |
| | | caactttcgcgttttactcgcgctctaggcgatatacttggcaattatgccgatgataagattgctcgcatttcgcttttacactc |
| | | aagtgcagatgaaggtgaaaaacgtcaagagcgtaagcggcacttaggtcgttttcctaccggtattggccttacttcagaggcta |
| | | aatttgatcgtttggagatcctcttaattcctgatgtgattgctttggtgcaataccacggtcaacctaattctgatagtgcatta |
| | | accctgccgattggttatataactgttgagccagagcgtttagaattacttaaaaaactaatgattaagcgaactgaaggggctgt |
| | | tgcaaccattacttggtctgaatcaaaatttgaaaatttagcttcgctattacctgttgagtttctgattaaactgaataagaaaa |
| | | gcggtgaagatgtggatgctgcaataaaaaaaatgcagatctataaccgtgctgaaaccgcacgggcaattttatcgctacgcaag |
| | | tagcatttatattgcaacgaataaatttttctaggttgctatgaactagctaaagggcaacaaatagataaacggcgttattcatg |
| | | tcaaatgagataatgttaaattgatagggatttataccccgccggccattttgaatggtcggagttgttataaacgtta |
| | | (SEQ ID NO: 50) |
| 54 | pLG056 | cgtgatgaatgaagcggctaaatacattaatgataattataatttaattcattaaaatcagtaatatataaatataaaagttgtga |
| | | aatgtgatattcgtcaaagcatgtcaaaaagttttgactgttctttaggcatcattcgcaattgtctaacaacttgataggatagg |
| | | aacaatctcaaaaaggaaaatgacatatggcatacgaagctcaaatcagccgtactaatccagcagcatttcttttcgtcgtcgat |
| | | cagtcaggttcaatgtccgacaaaatgtcttccggccgaagcaaggctgagtttgtcgccgatgctcttaatcgaactttaatgaa |
| | | cctaatcactcgctgcactaagtctgaaggcgtacgtgattatttcgaaattggtgttttgggttatggcggtcaaggggtttcta |
| | | atggtttctctggttcactgggaggacaagtcctcaatccaatttctgctctcgaacagaatccagccagagtagaagatcgcaaa |
| | | cggaagatggatgatggagctggcggaatcatcgagacagcaattaagtttccagtatggttcgatcctattgctagtggcggcac |
| | | gcctatgcgtgaagccctgaccagagccgccgaagagttggtgacttggtgtgatgcccatccggattgctatcctccgactatcc |
| | | tgcatgtgactgacggcgaatcaaacgacggtgacccggaagagattgccaatcatctacgacaaattcgcaccaatgacggtgaa |
| | | gttctgattcttaatatccatgtcagttctctcggaaatgatccaatcagattcccctcctcagacactggcttaccggatgccta |
| | | cgctaaactgcttttccgtatgtccagccctcttccggaacatctggtgcgtttcgcgcaggaaaaaggtcatacggtcggtatag |
| | | aatctcgtggattcatgttcaacgctgaggctgccgaactcgtcgatttcttcgacatcggaacccgcgcttctcagttgcgttga |
| | | ttcagcaatgaaactggagttcttagggacagttccgaaagatcctgaataccctaaggcgaatgaagataaatttgccttctccg |
| | | aagatgggagaaggctggcgctatgtgatggcgcgagtgagtccttcaactcaaagttatgggccgatcttcttgctcgtaaattt |
| | | actgcagatccgaaagtaaatcctgaatgggtagcatctgctttagcggaatattctgccacgcatgacttcccttctatgtcctg |
| | | gtcccagcaagcggcattcgaaagaggcagttttgcgacactaataggtgtagaggaatttgaagagcatcaggcggtagagattc |
| | | ttgctattggagatagcatcaccatgctggttgattgcgggaaactcatttgcgcatggcctttcgataatccagaaaaatttaat |
| | | gagcggccaacactgcttgctacgctgtacgctcataacaatttcgtcggtggaagcactttctggacacggcatgggaaaacttt |
| | | ttaccttgaaaaactcacccaacccaaactcctctgtatgacagatgcgctcggcgaatgggcactgaaacaagcgctggcagagg |
| | | attctggttttatcgaattactttcgctgcaaactgaagaagagcttgcagagttagttctgagagagcgtgcagcaaaacgtatg |
| | | catatcgacgactcaacgctgcttgtactatcgttttaacgcggaaagtaaagatgccttacccatctcttgaacaatacaaccaa |
| | | gcgtttcagctacatagtaagctgctaatcgatcctgaattgaaatctggtaccgttgccacgacagggttgggtctccccctagc |
| | | catcagcggtggctttgcactgacctatacaatcaaatcaggcgctaagaaatacgccgttcgttgctttcatagagagtcaaaag |
| | | ccttagaacgccgttatgaggctatatccaggaagatttcaagccttcgctctccctactttctcgatttccagtttcagccccaa |
| | | ggggtcaaagtcgaaggaatatcataccctatcgtcaaaatggcatgggccaagggagagacgctaggagaattccttgaggtcaa |
| | | caggcgttctgcacaagcaatagcgaaactatctgcatcgattgaatcacttgccgcctaccttgaaaaagaaaaaattgcacatg |
| | | gtgatttccagactggaaacctgatggtctccgacggaggtgcaaccgtccagttaatcgactatgacggcatgttcgttgatgag |
| | | attaagacattaggaagctcggagttggggcatgtcaattttcagcatccccgtcgtaaagcaacgaatccgttcaatcacactct |
| | | ggatcgtttctcactaatttcactctggctggctcttaaagccttgcaaatcgatccgtccatttgggataaatcaaattcggaac |
| | | tggatgcaatcatttttcgagctaatgactttgtagaccccggttcatcttccatcttagggatgctatcgggaattcaacagctt |
| | | tccacccatgtaaagaattttgccgcagtctgcgcttcagcgatggaaaaaacgccttccctcggtgacttcattgcaagtaaaaa |
| | | cattcccatatcgctagcttcgatcagtatgaatggggatattccagtcagcaggctgaaacccggttatatcggtgcctacaccg |
| | | tcctgtcagccttggattacagtgcttgccttcagcgagttggtgataaagttgaagttatcggaaagattattgacgtcaaactc |
| | | aataagacccgaaatggcaaaccatatatctttgttaatttcggagattggcgcggtaatatctttaaaatatcaatatggagtga |
| | | aggcattagcgctttaccttcaaaacccgatgcctcatggatagggaaatggattagtgtaatcggccttatggaaccgccttacg |
| | | ttagcgggaaatacaaatattcacatatctcaattacagtaacgactatcggtcaaatgaccgttctttcagaaccagatgcccgc |
| | | tggcgtcttgctgggccaaacgaaagtcgacaaacattaacttctactagcagtaatcaggaagccttggagcgcattaagagtaa |
| | | gagcaccacttcaactcctatgcccatgaacactaacgccacaactgcaaatcaggcaatccttaacaagttacgggcttctacgc |
| | | aaactgtagcggcagcaagagcgcaaactcagcatgtagtacctaataaatcatcaacgcattatgtggcaccgacgggaacatca |
| | | gcttcgcagccagttcaaaatattccgagccctgctagtacctcaaagcagcaaacctctcaaaaaaatatagttacaaagatttt |
| | | gaaatggctttttggatgattggtacttgtaaagaacaagcgcaatttcagtggccgtatcacttgcgcttgaggtgcctgcgggt |
| | | atgatcttgcgacatacaccactaaaacgaattcgtggcggcacttttagcctgcccctgtgttttcccgaggatttac |
| | | (SEQ ID NO: 51) |
| 55 | pLG057 | ggggcgaaaaggggaatgccggtcattgccggacgagtgcaccttaaaatgtgcggcagggggcgcccgcgggctgatccatttgg |
| | | cagaatggccgtgcatgcgacgatcgagcgcgggagacggctgaccctgatggacaaacgcgctttgagcgagcgggacatctgca |
| | | ctaagttcatcacgtcgtggcttgacagatgtttgccttgaccggtcgaatagccccattcggggccgtgtactttgcaaatgggc |
| | | cgaggtgcccgaaaaaccggtctggagccaggacaagaattacagtgcgcgaaccccaccggttactcacagcccgcttattggag |
| | | ttgatcgaaacccatcccgaaggattgcgactcgacgaggttcaggcgcgtacgcgtgttgaagggtgtcgcgcgggagtcgatga |
| | | tctcgcagcagcgctactcgatctccagcaccaaggtcttgcacatataaacgcagcccggcgctggtttccgaagcgggcggcga |
| | | gtgtacgaccatcctccgcagtcactggttcggatgacgtggcgggtgcagggctggtgctgcaggcgctaccggcgcgcatcact |
| | | ggcaacgatatggcggtagcaccagcacctgcattgagtgctaccggcacctcgctcaagccgacttggggcctgttacgcagcct |
| | | gctgccgtattacgccgaggcgctagcccgcaatgaacgggcgttgctactcggaacgcctgagcgctacggcgagcagttcctgc |
| | | tcgtggcaccacgcggccgatggtggccagcagcagggttaggctacgggctagaactctcgcgtacgcatctgccggttgctttt |
| | | ctcaccgcgttagcccgacgcacgcgcgaaccgattcatgtagcctaccccatcgcgctggtgcggccccgcgacgccgcgcgcag |
| | | cccctttctgttaccagtggcaactgtggcagcggactggaccctcgacgccgagaaactgcgcctgaatctgccggcccaaacgc |
| | | cggcgatcgaatggtcgtgggtgcgcggacagcgccagcgcggacgccagattcgcgagttgctcgatgcacttgatgtcaatgct |
| | | gacgacgaagtctggcgggcaggctccttcgtcgactgggcgaccttcgtcgatcgtctcgctgcaaccacccctaccgaggtgcg |
| | | cacaccgctcgatctcgctcagcccaacaatgagttggattgtggccaggcgggcggtatttacggggcgttggggctgttcctgt |
| | | cgagcgaattgcagttcgcgcgcggggcggtgcgtgatctcaagtccatgacgcagtggtcagatgacgagctggccacaacggcg |
| | | ctggctgcgtgcttcagcgatgccatccacaaggcaccgaatccggtcatcgttccggtgctggagccgcttgtgcttggcgagga |
| | | tcagcttgcggccgtgcgtgccgggctaaacgatcggctgaccgtggtaaccgggccgcccgggaccggcaagtcacaggtcgccg |
| | | ttgccctgatggctagcgcagcgcttgtcggtcgcagcgtcctgtttgccagccgcaatcatcaggcgatcgacgcagtcgtcggg |
| | | cggctggccgaagtagttgaagaccggccgctggtaatccgtgccaatgcgcgcgaaagcgatgacagcttcgactttacccgtgc |
| | | gatcgaagccatcctcgcgcggcccggtggtgagaggcccggcgaagggctggctggctcgatcgaagtgctgacgcggctcgatg |
| | | cggcacggaccgctgcgatcgaacaggccgccactgctaaccaagcgatcaacgaactcgggcggctggaagcagcgatcggagat |
| | | ctgacggcagcccttggcatcgacgcagccgctccactaccgcgggatctgcccgctgccacacgacccttgcatagttggctaga |
| | | gcgcctgtttgcgccttgggtacggtaccggcgactacaacggctacggcgtctagcgctgggatggggccagcttggttttggcg |
| | | agtgcgacgaatcgacgctggagctacacgaacaacgtctactcgacctgcaggagctggctgcgctgcgggtcgagcgggatcag |
| | | gcagaggcagccgtgcgtcaactccgttcaaccggcgatccgatcgcgctcggagagcggctgtgcgcttcatccaaattgcgtct |
| | | gcaggggctcgccgaactgcttatcgagtgtgcgcctgaagatcgccgtgcgttgaccgcgttgcgcggcgatctggctctggcgc |
| | | gcggtgatggcgccgccggtgctgcccgtgctcgggaactctggtcggctcagcgagccctgatcctcggccagatgccgctatgg |
| | | gccgtgtcaaacctcggcgcagccagccgcattccgctggtacccgggttgttcgattatgtggtgcttgacgaggcatcgcagtg |
| | | tgatatcgcttcggctttgccgctgctggcccgggctcggcaggcgatcgtgattggtgatcccgcgcagcttacgcatatctccc |
| | | aagtgcgccgggagtgggaagccgaaaccctgcgcaatgccggcttgatgaggcctggcatcggcagctatttgttctcgaccaac |
| | | agtttgttccatcttgctgctgctgccgccggcgaccatcacctgctgcgcgatcacttccgctgccatgaagatattgccgacta |
| | | cattagtgccacattctacggcaatcgcctgcggccattgaccgacccgcgtagcctgcgggcaccagtcggacaggcagccggtt |
| | | ttcactggacgaccgcgcccggtccgatccaaccagcccgcaccggctgctttgcaccagccgagatcgaagccatcgtgcacgaa |
| | | ttgcattggttgctgggtgagggcggcttcactggaagcattggcgtagtcacatcgtttcgcgaacaggccaaccgtctacgcga |
| | | ccgcatcgagcattgtttgagtgccgaggcgattgcaagcgcacgattggaggttcacaccgctcacggcttccagggcgatgcgc |
| | | gcgatgtgattctactcagtttatgtatcggtccggatatgccggctggggcgcgagccttcctgcacgacacgggaaatctcgtt |
| | | aatgttgcggtgagccgtgcccgcgccgtttgccatatcttcggcaacctggagtatggagctcactgcggtatccggtatgtcga |
| | | ggcactgctggcacggcgccatcgaacaggcgatgccactgccagtttcgaatccccctgggaagaaaagctctggcgcgccttgg |
| | | ctgagcgcggtatcgagacaacaccacaatacccgattgccggtcgccggcttgatctggcattgctgaccgacagtgtgcgtctc |
| | | gatattgaggtcgatggcgaccgttttcatcgcgacctcgacggtcggcgcaaggtgggtgatctatggcgagatcatcaattgca |
| | | ggcgctcggctggcgggtcgtgcgcttctgggtttacgaactgcgggagaacatggatggttgcgtcgaacgcatccttgtccaca |
| | | tccgaagcaccgattactgagcatcaccgttccccaccagcagcagccgtgccaccagcgaattggcggcgaatgcaactcgtgct |
| | | cgggctggccggggctctggcgctggctagcctcgtcactgtattggtgggtgtaatcggcgacgccaccgaacgcgagagttggc |
| | | gagtacggcgtagcgagcatcaggaggtgctgggcgcgctcagcaccgcacgtgcccagcttgatgaggaagtcgccaacctacgc |
| | | cgtaatcgtgctgcgctcgatgcagacctgaatcgtctccggaccagcgccgaagctgagcagggcggcgcagcacggctgcgtga |
| | | ggaagtcgccgcactacgccaggagctcgccgccggccgcgccgagttggctgtggctacgcagcggcgcgacaccctgcaggctg |
| | | cagtgaagacggccgatacgacgctggcggaactgaacgcgcgccgcgatgaggccgagcgtcagaccggtgaggcagcagaacgc |
| | | cggcgggtcgcggccgaagccgagcgggccgcgaaggcccagcagagcaaggccgaacaagcccgcgacagtgcggttgcacagca |
| | | gaaggaggctgagcggcgcatcgagcagatccttcaggacctgaaaaccgccgaagaacgagtaggtggactgcgcacgcaagagg |
| | | ctcaactaaaagcggctacaactgcctccactgccgaacgtgaccggctggatgctgaagccaagcggctcggactggagcttgtc |
| | | aagctcgatcagcagcgccagcagcttgagcgcgatacccgtactaccgccgaaactcgacggacggccgaggggctccagcagca |
| | | gctcgaccaagcgaaccgggatctcggtaccgtccgcgaagccctgaagaccgcgcaggggcagctagccgaaacgcgcggccagc |
| | | agacccaactcgccgacgaactggcccggctgcgcgcacagaaaaccggcctggatggcgtgatcaccgcggctgctaacgctcaa |
| | | gcggaacttgacaaactgcaggctcagcagaaacgggcggagcaagcagcagaaacgacgcgtctcgatgttcgtcagctcgaatc |
| | | tcggaaaacggcactggaagccgacatcatcaaattcaccgccagcggcaaggatttggaaaagttccgtgccgaactggctgata |
| | | ccaatgcagaactcgaacgtctgcgtcagcaattggttgaggcacggagccggcgcgagactatcgcgattgaagtggaacgccta |
| | | acgcaacagcgcggcgaactggagcgcaccatcggttcactaacgccgcgagcgcaggaggccgaagcgctacggatccggctcca |
| | | gcaagacaacggcactttgctcgccctgcgcgagcagattgaacgcttgcgcactgaacgtgacagcttgcagcagccggtcacat |
| | | cttccatgcatgtccccggcgacaacgccgcggcacgctgatcaaggatcgcgctgatggacacgaacaccctggtctggcttgca |
| | | tcgggtggcacgcttgccggcatcgtcagtgttatcaccgcattggtgtgcggcatgcactacggtgcggcgctacgccgcatacc |
| | | ggctgcggcctttttggaagatatcgtcgcacgcgtcgcaactcgtcgcgaggaactcgaacggctggatgcccaattgggcgagc |
| | | gccacaacggcctccagggcctgcggggcgaaacggagatgctgacggcccgccgggatgccttggcagcgcaactgcgcgaactg |
| | | caggaggacctggttgcactcgatgggcgccgggccgacatcgcttcggtgcgcgatgagttggcggaagcacggacgcaacttgc |
| | | catgctcgtcagtgaactgaccgaacggcggacgcagcaggagcaactcgaacgcgcggccgaacgtgcccgtgcacaactgtccc |
| | | tgctcgaagaacgccggagcgagatcgaggcaatcgatacagccgagcgcgaagcacggatacggctcaccgaggcgcagacggaa |
| | | ctgggcaccgtcgtccaggcgcgggaagcggcacggcgtgaagccgaggcggcagcgcgcgacagggagatgctggcaacgaacat |
| | | cgaccggctcaccgatgagcgcaacgaactgcgcgctgacatcgccagtctccaagccgaacgcaatccgctgtcgactgaagttc |
| | | agggcctgcgccggcacttggagcagttgcatcttcagcagcaggcactcgacggcgatcttcaacgcctgcaatccctacagccg |
| | | gtactggaagataaaatcagcggcctgcaacaggaagttgttacccggaccgctgaactcaaagaccttcaggccgaacgtgatcc |
| | | gctgtcgactgaagttcagggtctgcgccggcacttggagcagttgcaccttcagcggcagacactcgacggcgatcttcaacgcc |
| | | tgcaatccctacagccggtactggaagacaaaatcagcggcctgcaacaggaagttgttacccggaccgctgagctcaaagacctt |
| | | caggccgaacgtgatccgctggcagcggacattgatggcctgcgtcggcaactcgaaccgctgcgtacacagtgcgacgaagtcga |
| | | agcggaactcgcccgccgccgcgccgaactcgccgcgatcgagcaggagatccgtaccaaaggcggtggtagcgtcggcaacccgg |
| | | aagacgtgctcgccgatctcgaacaggcaccggcttgtctggtcggcgacggcggcaggggaccgttgatgccgaatccgcagcgc |
| | | gacgacgacgaaacagcaatgctcggccgcgtgcggacacaccttgatcggctccgtctgcactttcccgagcgcactctttatgc |
| | | ttttcatactgcgctcaagacggcaacgattagtccgcttacagtgctggccggcatttccggtaccggcaagagtcagctgccgc |
| | | gccgctatgccgaagcaatgggtatccatttcttgaaactgccggttcaaccacgttgggatagcccgcaggacatgctcggtttc |
| | | tacaattatttggagaagcgctacaaagcgaccgaatttgcacgggctctggtgcatttcgacacgtacaactggccgcttgcccg |
| | | gcctttcaaggatcggctactgttgatcctgcttgacgaactgaacctcgctcgcgtcgagtactacttcagcgagtttctgagcc |
| | | aactcgaaggccgtcccgccccgggcgatcgcgatcctgagcacatccgcagttcggaaatcgtgctcgatactggcggcgttggc |
| | | ggaccgccgccacgcatctatcccggccacaacctgctgttcgtcggcacgatgaacgaggatgagtcgacacagacactttccga |
| | | caaggtgctcgatcgcgccaacctgctgcgcttcccgcgccccgaaaaactggccggagaaacgctggcgagcggcggcgagccgg |
| | | cggaaggcttcctgccggcctctcgctggcatgcgtggcggcgcagttttggcacgctgccggcaacgctgcgcgaaccagtcgaa |
| | | cgttggatccacgatctcaatgagcatctagacgggctgcatcgaccgttcgcgcaccgtgtcaatcaggcgatgctcgcctacat |
| | | cgccaactatccgggtgtcgccgagccgatggcgcaaaccagtcctctggatcaggcccgcattgcctttgccgatcaactcgaac |
| | | agcgcattctgccgaagctacgaggcattgacctgggtgactctggagtcacccagcacctcgaccgcatccgtgcgttgatcgac |
| | | aacgagttgcatgatgcaacactggctcgcgcctttcagcgcgccgcgcaagatgacggcagcggcaggccgttcgtgtggaaagg |
| | | cgtacgccgtgaatcgatatgatcccgctggtgctggctatgccatggggactactggcacagactccgatcgccggccagccgac |
| | | gcgccgaccgttacatgacggtgaaacggtcgaactcgatgggcggtacggtgccatggtggcgctacccgagcggaccgacctgc |
| | | aactgggcagtcggcgctggccggtgcaggtggaaggtgccgcctttgcctggttcgagggatcctttcggttggtgtcgctgccg |
| | | actgcagccttgaccagcgaacgtcagatccggttcgatcttctaacggcgggcgagtctgtgctgagtgtcgggctcgtgttgcg |
| | | taatcatctactgcgtccgcgcggagccggacgtgacgatccggccgccgatgcattgcacacctttgtgttgcaggttctcgacc |
| | | gcatccgtgaggccgaaccgtccggtgccggagacgattgggatgatctcggcaccggttgggcgcggctgcgcaccgcctggctt |
| | | gagcgcgatgcgcagatcgaagaagcgcgccgcgatctgatcgtcgaacatgctgaacaactcccggcccacatcaca |
| | | gaaatcgctatccacccgcgtcgggtgctcaaacgcacccgcgagttgctgccgatcgatcgtatccaggaactcgacaccgcctg |
| | | tctcgaatggctgatccggcagcccggcgttaccgttgccgaaaaggccggtccgcgccagcgactgctcggcatcgcgcgcgagg |
| | | agcatctcgatacgctcgaaaaccgggtgctgaaagatttcctgcgtctgagcgtcgaggctgccagcgtctggcagcgggagaac |
| | | cggcgttttcacaacagtgagcgcgcccggctggtcgggcgttatctcgcgctgtgccgcatgcatcatcgcgaactgtgcgcggc |
| | | tggcatcggtgaccccatgcccccggtcgctccgaatttcgtgctgcaacaagattcccgctaccgcgtgatctggcgcgcgtacc |
| | | gcgaactgttgagcgctgagcagcgtatggacgatctctggcgctggcagtgtcggttgtggagcgacttcgctcggcttgtcgtg |
| | | gtgatgggggtgcaagagttgtgcgacaagccgagtgcgctctcgcccctcttcgtgcgcagggaacaggcaagcggacgctggtc |
| | | ggacacgctcggcctgctcggtgtattcctgatcgacctgaacggcaggtcgtatgtggcggaagtctgtgatgcgagccagttgc |
| | | cccgaaacgacacgtcacgagcgaagctggcgtcctggcagtatgcactcggttgcacagcactcatccgcctcatcgatttgtgg |
| | | agtgggcattgtgcgagcctgtgtgtctgggccatgcatagcgctacagccgagacgcttccgttgaccgagttggtcgcttcagc |
| | | cgatgaagccctgagtacggccatcagacaggaaggtctgcgcaacggcgagcaacttcgggcacgtggactggtgatccgctcgg |
| | | cgccgccgggaaagaccgagtacgccacccaggctgggcaggtctacggactgacgctggccatcgggtcggaacatatccgcgag |
| | | gcgcttggcgagtgcactttgatcctgcaggacagtctggagcgcctgtttgcatgagcggagtgcacggcattgatctcaatggt |
| | | gtgctcgattgcgtggtgcgcctcgatcgggcaccgcgaccagcgccgacaccgccggtgatcgtctccggttcaccacagggcct |
| | | gctgacgggagccgcggcactgcaatcgccctgcggccgacctggcatggaagccgaggaaggtatccgcctgccagtgctggccc |
| | | tgctgcacgcgctcagtggtgaggggcggcacgatacgcacgatacggccgtgctgctcggccgacacctgcgtagcctgttgtcc |
| | | gatgatacgcatgctgctgtcgtcgcagtgcctgacacacctggtttcgacgaacgagctcgcacccggctgctggatggcgcgct |
| | | acgcgccgggctcgatctgcacctactatggcgcccggtcgcagcgttgcttggttggggcgaaacactgggaaacggcgaactcc |
| | | aagccctgcacggccggacggcctgcgtcgtgcagttgttgccggacggcatctcgattggcgatttcggcctcgaatgcgtggtg |
| | | cagggtggccggccgacgttagtaccggtgcgccggcgcgacggcgaacgtcaattttactcgtggagcggtggtggactggttgc |
| | | actgctcgcgcgcgaagctggaaccgacgaagccagtctgtgggtcggaccgtgggtatggaaggtcttgcttgggcagcctgcag |
| | | aacgcgaggtgctggccgacccgcatgcaccgggtggttggcgactcgccagcggtccttccacactgtgcggcgccttagccgcg |
| | | gagttgcgcacaggcctgcgtatagcactcggagccgcgcgctcggcactgcgcaatgcagcggtcaUctgatcgaggggcctatc |
| | | gccgatgcaccgcUtcggacgcaatgcagccaacactcgcgctacgccagatcgtggctgcggaactgaccgtggtgctcggcccg |
| | | acggtgtccgcaagactcgtcgccatgccgctcgccgatgctctaattgccagaggggccgctatctgtgctgcgcgtcaagcggc |
| | | gcggcagatcacgtattacgatttcctgccgatgctcgaaatcaatgtgctgcaggccggagagcatgcgttcgttgaactcatcg |
| | | gtcgcgaagagcgcatcgcggggggcatgagttacacgaatacgttggccgatcgcttcaccgttgccgcaagcacgcgctcgctc |
| | | gagttctacctgctgaaagaggacgaagcaggcgctcgtcacagcgaaacggtgctgccggtaccgccggcagccgacgtggaaat |
| | | cagcctgcacgtcacgcagacacccgctcaaggctacgcacgcgtggagatactctcggccgtccggggcgcgctcggtgaagcac |
| | | cgatcctgctcgattggtcagcgatgacagagattgaaggctcgcgcgaggatattctgcgcgaactcgaattcgaggggctcggc |
| | | tatccggacatcgtaccgcaacgtgcacatcacctgctctgggattaccagcgcagtgacggcatgactatcgctgccgcgatgcg |
| | | ggccttcaattgtaagcctatcctaagttcaccgcgcaaccagtacaatcaattggttaaacaaacgcgcgcactcgtcgggctgc |
| | | gcagcaatctgttttttctgacaaagggcaccagttctgatcgtagtgcttacaccgccgtcgattcggatggccaattgccacct |
| | | ggaatcgcgccgacaatccaacaggaattcgaaaactttcgagtgcggctcgacacggattttgccgcaatcaccagcgtccgtaa |
| | | tcgacaagatatcgcaacccggcgtgaattggcgcgactgggcgcctgcttgtatgcagcgtgtcctaatgcaattgttcattact |
| | | tccaacgcattgtcgcacgtagcgccgatgacctgacactggtgttgcatgccggcaaagtgctgagcaccgaaccagatcttgac |
| | | agtcttttccattattgcgcgtctcgctacgatgaagccatccgcgctgtcaagagactgtcggtccacgtggtacgcgcggcagg |
| | | cgatgctttggcttatcatgaaaaagctggaggcattcttgataaccgaagcgctgacaagttggctgaagctgcgctcctattgc |
| | | taaaggaggaaatccaggcacataattacaaaatacgattccgtgccgccgcgcgactcggcctatttctgttacgccaccggcag |
| | | cggcggcgcgatttcctgcatccgagtagcgctgacacggctaatcgtcggcgtgccaaagagttcgatgccctgttgatccaggc |
| | | tatcgcatcgaagcgccttaaccaagatctggaaaatgccttggaagaaatccgtgcacaaatccgatatcgcggtacaaatgcga |
| | | tcgttgatatcgatcctgacgaagatggcgagattaacgagaacgaagtggagtagaggctgttgggcacccgctcgccatccctg |
| | | tcgagcatcccggcttcgcgggcgcccatcccgtgcctttacggcgtgttcaacggccccggttcgccctgcgtatcgggctcctg |
| | | ctacgcccgtcgagacgcgctgcgcagactcgacgctcaaatggcttgacgccattctccctggctacc(SEQ ID NO: 52) |
| 56 | pLG058 | tcgcgatcaaggggtgagcaggggataaacgcaaagacattgaagttgaggagaatttagttgccttacctgcgaaaaatctgagc |
| | | gatcttgcattaaagattttctatctcaggccgatgctcataagagcatttcctgaatttcaccctttttttgctcgccatccctc |
| | | tgcgaataaggacaccgcgccagatatgtcactcatcacccatacattagaaaacctcacaaaagccttgcgtactgcgttgcgtg |
| | | tctcaattgaatgcaatgagcgcagcgaaaatacccataaaattttaaacgtgttacgtcaggttgagctgacgctgatgctgcat |
| | | caacaacctatctatgccattgccggtacgcagggagcgggtaaaaccactctggcaaaaagcctgctgggcattgacgatagctg |
| | | gcttgaggcgaatccgggacggggcgagcagataccgttatttattgagcaacggcacgatgttcagggtgattatccgcaattta |
| | | tttatgtctgtgctcaccacaaaaccggtgaaatttttgacagccagccgcgcagtggcgatgagctgaaacagatgctgcgtgac |
| | | tggtcgcaaatggtgaatcaggagatagaagggggcaaaatcctctatccgaaattaatcattaataagtcagacagttttattga |
| | | tgaagagatggtctgggcgctgttgcccggctacgagatcagcaacagccagaatcatcgctggcagggcatgatgcggcatgtca |
| | | tggtcaacgccagaggcgtgttgctggtcactgacccgacgttaatggcaaatacgaaccagagcctgctggtgaacgatctgcgc |
| | | agtgtgttcgccgatcgttctccggtgattgtcgtgaccaaaacagaaagcctgaacgatgcggagaaggccgaggtaaaagcgag |
| | | cgctgccgcactttttcatgagacctcctcaccggtggtcgctgccggtgtcgataatcaagcgcagtggataggtgagctccgca |
| | | ctgcatttgctgagggtatccataatagcgccgcgtcagaagcggccgcgatcgaacgtttgatgactctggtcaatgacgatgtt |
| | | gcggatattattgataacctgaatctgctgtacgcggagcaggacagtggcgaggaacgtaccgtcgctattcttgaagcgttcga |
| | | taaagcagccgagcgctatgaacagcaactgcgtaaagccatcaaacgagaaactgacgggcatcggcaaaaagccactgaatctt |
| | | gccagcgccgttatcaggaagaagaagaagggccggtcaataatttaaaaggactcggtcgtcgtctgatgtttcagggggcggag |
| | | attgatcgtgaacgcaaaaatcgggtactggacgcctggcaaacccgctttgagcagcaatctctggccgatcacaatatggtcgc |
| | | gctggaaacgctcaaccgtcgtgagttgaggcattacggtctttcacaggagacgctgtcaccccaacggttgacctcgcccgcgg |
| | | cgacaatgggatatttgtcggtggctgaggaggataatttttcctcgctggcccctttgcgccatctgctgggatcggctgcaaca |
| | | agggatgcgccgccgcagttagaccagctttccacggtattaaaagtgctgcctgccatgacgatggaatatgcgcgcggttgggt |
| | | ggcgatcaaccaggcgatgcccgcagcgtcagagctaaccagcgagttgcggccacaacaaattctcgacgcgatttttagcgcgc |
| | | agagtagcatccacccggtgaaaaccgcgctgatggcgtttatcggtgccgacgccgcggacggcacgctggatggcgaagtgggc |
| | | actccgcagaatgaagatagcggcgtatttacgcctgtcgcgatagcaggcaaagcgatgctggtcggtgcggcggtttatgcgtt |
| | | gtatcaggtggcgggcgtggtgagtgagagtgataaagctcaggcctggtatattgaacggatgatgaaggaactggcgcaatata |
| | | atgaaaacgtcatcatcgagcgttatcaggacacgatgggcgatctgcgtcagctgattgaaatcaacctcaaccgtttatttggc |
| | | gtgcaggatgtcctcacgcagaaaagctatctctggttagctattcagggactcacgacggtacaaaaggaagcccggcagtatga |
| | | agccagtatcaaacaatatctggcgtgatatttgccatgagcgttatcgatgggcggaaaatagctacatcaacctgctgcgtcag |
| | | gttgatgccgagcggttaatccagcctcatgcagacatctcccgccagatatcggtcattgtctatggtccgacgcaggtgggaaa |
| | | aacctccctgattctgaccctgctgggcgtcagggatgactgttttaaagaacttaaccagctgctgcgtggtgggcaggcattag |
| | | gtcacgcgtcaacggcgcgaacttaccgttaccggatatcacgggatgatgcctggtattttagccacaaagaccagggaacaacc |
| | | gcctggtcggatagcggggcggcagatattttcgccagcctgcgtgcagaggttcaggcgggcaggcgctactttgacagtatcga |
| | | cgtatttattccgcaacgtttcttccatcctcagcagcggcaaaatggtttgttaatccgcgacctgccgggtattcaggctgcgg |
| | | atgacaatgaaagggaatatgtgactcagcttgccagccagtttattcgttctgcggatgtgatcctgctgaccggcaaagcggat |
| | | tatttaggctttctgaaacccgaggagttgggtaatgacctactggctgactggttctggcagccacatcgctacaaaattgtatt |
| | | aacccggacttttagcaacagttccattcgggaaatgttgcgccgtgtttcccccgataaatcctggctgcaggcttatttgtttg |
| | | agcaaatcaatacgctggaattgcaacttccggcggagatgcgtcaacacatttatccgctcgaatgcggtcactcctggcaaacc |
| | | ctgattgaggggggtgacgattatgctgactattgccaacggttgcgtgagcagatattaaccgacctgcgccatcatatgttgca |
| | | ggcggtccatccactttctcgtttacgtacgggatacgccttacctgaattaattatccgccaccgggacaagttgcagcagcagt |
| | | acacagcgctgcacagcacgctggacaaagaacaggaatattacctgcgtaaaaaagagcagctgtcgtctgtgcagactgaatat |
| | | tcccggcatctggcaaagagccagacacgactggacagattgcagcggctacgggaacggctgaataaaagacaggcgcgcaacgc |
| | | gcatcaatccatcgctgtgccaccgatgggcacaagaacggtcagtgccttactgaaaatgattgctgaggcaagagaagagatgg |
| | | cgcttcatccggcgttaaagcaccttcctgcccatttcgctgcgcaacagattaaccaccatgccttcacggcgattgagcaaaag |
| | | ctgcatggctatcatgcggataattatctctttgccagcaactataagcatgactatcaggaaacgatcaacgcgatcaaacaaca |
| | | cctgaaactgatcaccacattagccgctaatttccagcgtagtgagctggagagacacatcaaggaacatcgtcgtcgccagcaac |
| | | gtttacaacaccacaccacccggcgagacaaactcctgacggcagtgaccaataagcttacgcgcatcaatacgcagcaacaggaa |
| | | ttaacgcacagccatatgcgtgacgaggatcattatcagcagctgattggcgagagccgtcgctttcaggaactgatcagagtggc |
| | | gaaaaatgaacgagccaccctgattgaacaacacattaggcgtacggatattggtcaggctgagcgactggcctggctactcgctg |
| | | cccgtgcgttaaagaaagactacgaatatgtcagagcattaggagagtagtgcatgtcagtggaacatgacccggttattgcgcag |
| | | gataatgacgagcggatgctggatgaattggtgcaggaactgtttctgaccttgctgacgcgtgagctggcgcaacagaaagcggt |
| | | tatcgaaaccattaatgacaacgtctcgtatcaggctggtgagtcattaaaatcgttgaaacgggagatcaaactttccatcagca |
| | | ccctgtcgaatgcgcaacagcaatatcaggaagagcaggccatcgccagggaggaatacgagaagcggctggagcagcagactcaa |
| | | acatttgccagtgatgcggaaaaaaatcaccaacagtcacagcagcagatggcagcacttcggcaaggtgagcagcagctggctgc |
| | | acagttaacagatttgcagcaacagcatgccacacttcatcagcgctcaggtcagatgctgaatagcattaaatggctggtggtgg |
| | | ggctggggggcgtcaacctgctgctgtttgcggctgtcatcatgatgttttttctcgggcatcgataatcatccgcgcatgcaggt |
| | | ttgtccggatatggtgcgcctggtgcaccatgacttttctctggcacggataaacggacgcacaggcagcgaatgacgcgccctga |
| | | ataaactggcacaacttctgcattcatttcctcaggcttgtatacaaggccgcataccg (SEQ ID NO: 53) |
| 57 | pLG059 | cgcatctgtaatgcaaacttattagacttaatccctataatgcaatataaatcatattgttaccttgtggctcctttatctgattg |
| | | cacggatttatccctcgcgtacttattcagcatgatatagctgggtatcatgtgcctactcttaacctgaatgaaacttacaaacg |
| | | ttcgtggtatccacatgctaagtgaggctgagatagcaaaatttctcatatggttgctgcccctaagatcaacaacgcactgagca |
| | | tgactctctggacaaggtgccacacaccaggcgcacgtctaaaaggaaatatacatcaaatacctgattgctaagttataccaagt |
| | | ggaaatcgggtatagtaggtcaaaacgaaagcgtgtcttaacactgcatattaacgatcaggaaggtcttagcatgtcaattaata |
| | | tcaatacgttgcataatcttcgtcgcgcgttacttactgcgctggagctctcgattgagcacaatgaagaaacagaaaatgtcgat |
| | | cacattactgatgttctgcggcaggtggagttgacagtacttttgcagcaagaatccatttacgccatcgcaggtatgcaaggggc |
| | | aggtaaaacaaccttggcgaaagcgatccttggtattgatgatgaatggttagatgccaatccgggtcgtggcgaacaggtaccgc |
| | | tttttatcgaacaggtggatggcgatccctccgattttccacaagttgtctatcagtgcctaaaccttaaaacaggcgaaattgct |
| | | ccgcaaaagggcgagggtggggagcaacttcaaagtctgcttcgcgattggagcagtattcgtcgttatgaaaaagcgggctttaa |
| | | actgctctaccctaaattgctgatcagtaaaaaaaactcgttcatcaatgagcaagtgacttgggcgctgttgccgggctatgagg |
| | | tagccacaagtaaaaactatctctggcaggatatgatgcgccacgtattggttaacgcccgtggtgtcatgttcgtgaccgatccc |
| | | tctctcttagccaatgacagcaaatccgcagtgctgcaagatttgcgagataacttcaaggaacgcggcccagtggtggtcatcag |
| | | caaaacagagatgctcggagaacatgaaatcaaacagctcaaaaccagtgccgctgaacgtgttttccccaatgttgggatgaaaa |
| | | aagaggatatcgtagctactggttctggtaataacgacatctggattgatgcactacgtgacacagtcatcaataagctcaccagc |
| | | agtgcggtatctgaagcaattgcactagataacttcatgggacttatccgcgaagacgtggccgaaataatcaataatctgaagat |
| | | attggcggatacacagcagcatcacgaatccatagtggatgagatcctagacgttttcgatgaatcagcctccacccatgagcaaa |
| | | aattacgtgaagcgatcaaaaaggagacccgtcagcactttactgatgcgcttaagtactgtgaaaaaagctataaaagagaagag |
| | | gtaggttttcaaaaaaacctcaaaattttcgcccgccgactgtcgtttcgcggcatagaagtggatgatgagcgcagtcaacgtat |
| | | tatagatgcttggaatagacagtacgaaaacatcagtattcacgaacataatttcgacgcactgacgtctgtgaatacccgggtgc |
| | | tgcgtgccaaggggctattgcctgtcgttgaaaatcagcaactattaccgggcagcgcagtcgggagaatggggtatctggttcag |
| | | gataaacaagcagagtactcaataatggatcctgacctgatgacgggtttgtatacactgctcaaaaagccgggcggcgctcatca |
| | | agcaccgccgcctaaaaaactcgctgcggcgctggagattatgcctgctttaatgctggaaaacgctcgtactaggttggcaatgc |
| | | atcttgacccggcctgcacaacccaactggcagaggagatccagcctaaacaaatttttgatgcgctcttttcgagcagagaacag |
| | | taccatcctattaaaacagccatgatggcgtttttgggtgctgatgcggcagatggaactgtagacggtaagagcacgccaaatac |
| | | cgaggggggattcgctccgctagcgctggtaggtaaagcggcattggtagcaagcgtggcttatggcatctatcaactaacaggag |
| | | ttattcgcgacagcgataaagcgcagatttattacattcgtcgtgtgatggaggaattgtcattccataacgaacagaccgttatt |
| | | ggcaattataaggagatgattggcgaattgcgtgattatattgcgtataacctgaagcaaatatttggcgaaacggatgccctggc |
| | | aaatcgaagcgccttgacgcttgccattaaaaatcttgttgccgcacaaaaggaagcaaaattgtatgaaactcacttccgaaaaa |
| | | tcctgggctgatctttgccaggagcgttatctgtgggcggaagagagttttgtcacgtttctacaaaaatttgacgcacagaggtt |
| | | gatccagtcggcagacaatgccaataggcaggtttcagtgatcctgtacggtccggcccaagtaggtaaaacctcattaatcctga |
| | | ccctgctgggtattcgtgatgactgcttcaccgagctcaatactttgctacgcggcgagcaggggctgggcacaatgtccacggct |
| | | cgcacctatcgctatcgcatggcgaaagatgacttctggtatttcagccatagggagtacggtgcaactcggtttagtgacaagga |
| | | ggcgaaagtcatttttgcagattttcgtcaggctgtggagcagggcgagcgtgaattcgatagtgtggatgttttcctgccgcgcc |
| | | gtttttttgatccgaagttacagagcagtgcccagttgctgatccgtgatttacctggaactcactcaaccaacgccaacgagcag |
| | | tattatgtcaacatgcttgccagccgatatcttgcttctgccgatgtggtactgctgaccggcaaggctgatgcgttggccttcct |
| | | taagccggaagagttagacaatgctctgctgaacgactggcactggcaacgccaccgctacaagattgtactgacccgtgcttatt |
| | | cagatgccacactccagcgttttatcaaacaaaaacggtttgataaaaaagcaatgcggatatttttgcttcaacagattaatacc |
| | | atggatctgggcttgcctgaaagcatcagtgaactgatttaccccgtggagtgcggtcattcttggctggcaatcaatgccaaaga |
| | | tgacgagtttgcccgccagtgccgtgatttgcggcgagatgtattgcaagatttactcgactctctgcaccaggcatcgaacccat |
| | | tatcacgcttacgttcgggatacgcgctgccacatatcattaaacagcagatagctgtcgaaaaagagctttacgagacggaaaac |
| | | gcattgctgcaaaaacagctctctcggctgggggaatatgttgatatgtacgagaaacgggtcagcagtaatagagataatcacct |
| | | gaggttacaagtaaagctgcaagcactattacaaaaacgtgaggacgcgttgagtacagattttcgtgaacattcgaatgcgtttc |
| | | aaataatttcgcaatcatctctcggttatcttaagtctcaaatttatgcatctcgtgaaacaaataccaaacgctggaacgatctg |
| | | ctggaaatctaccagcttccacttgaaagagtaccggagatgcccaatctagagcgggtcttaaaaagactaaacggctacttgtt |
| | | tgagacctattttcgagagaaaacacgtcagaatgatcagtatgagatagaagaggcaggctttaaagacgcaaactgcttaacgt |
| | | atattttccacgaacgaatcaaggttaagtttggtgccgaagagcgcgccttgaacaataagatagccaaaaacgagcgggcagcg |
| | | tgccgactggtgcgtatcgHgaacaattgtcgaaaaaaatggtgcacacgcagtcaagactcttccagatcaagcaggagttaggc |
| | | gtatcgttaactctrtattttcagagatataaagagagtaaaaacttttcgaaagtcattgtttcggcgaaaaatactcgagcgcg |
| | | tgaaatcgaatgcaacgctaaaaaaccgaatattacacgcagcgagcgtctcgcttgggtgctgatgtatagagcgttaaagaatg |
| | | attttgactacgtaaagtccttagatgaggagagcactaaagttgaataaaaatcttgctgtcgcggaagtgtccagcgatgagca |
| | | gttactggaccaactggtgcaggagctgtttttagagcatttgcgacgtgaactgggtgtgcagaagaagagtattgacgacagta |
| | | atgacaaactctttaatctcgaccgaaaatttgtcgctgaatttaaaaacgtgagcggattgcttgatacgatatccgacactctt |
| | | ggcgaacagactcgtgaactgaatgatgctaaagctgatgcccaaacacattatcgttctttgctgaatagtttggcacagaaccg |
| | | aacggacaccgctgctctgcaagatatactccagcaactaagtagtaagcgtcataaggaacaaggcgagcaactgcaacggatcc |
| | | aggaacagttgtttcatcagagcgctgaactccaagcgcaatactccgtgttgacagaacagaatgcagtgttaaaccagcagcag |
| | | gaggtccttcagaaacaacggttcactgctactctggccgaaatgcaagagcaaaacgtgacgctggcgtcacttacggaacagaa |
| | | taagtcgctgcatcgacagtttctcaccttagaagatgaacaacgtgcagattttcggacaaatagtcgctggggtaagcttgccg |
| | | ctggattctccatagcgaatacgcttatcctgataagcgtgaccgcactgtttatagttaagtactttctataaagaacccgcgtg |
| | | cacaactcttcttcatataaaatatcttttccaacagatattgcattgaggatttcttttattgctgtttatgaaatggctaaata |
| | | tcctccgacaaataagaacagtggcggatttttcatcctcgtctttttcagggag (SEQ ID NO: 54) |
| 58 | pLG060 | atcagggcaaggaccgttgcccatatgtgactggttttggtgtcggctatgtggccaggctgcgtgaaagctactgatcgcttttt |
| | | aatctaagtggtggatttatatgatcaatcattattgataaactcatgaagaaacctaatttatttaataaaattaaaaagtatac |
| | | gattagatattgcgggtgtagatatgactcaccacattaaaggtcaaggcagacatcaggtgacgttgctctctgacgtgcttgat |
| | | gattttgtcacagaagataaaaacacgttgaagagagaaaaatgaataccgcagaagactttaaccgcctctatgccgacgtttca |
| | | cgcaatattcagcagacgctgactgatatcgctgcacttcatgttgaaaatgaagagggaaagcagcagctacaatcgatggtcac |
| | | tcagttgcaatccctgcaggatggctttaaccagaagctcacgtggctgcaaaagcatgccgaatgggacaaatttaccctggcat |
| | | tctttggcgaaaccaacgccggtaagagtacgataatcgaatcgctgcgcatcttgtttgacgaagaatcccgccgccagctgctg |
| | | caaaaaaaccacaacgacctggaaaaagccgagctggaattacaggaaatctcggaacgactgcgcagcgacttagggcggatcta |
| | | tagcgatgtagtggataaaatcaccgatatcagtttttccgctctgcgtctgatgcaaattctcgacaatgaaagcgccctgcgtc |
| | | acaaacgggaagaggaagagagcaaggaacgcctgctggttgaaaagacggaaagccagtcgcgattgcaaattctgcaaaaacac |
| | | accagcgccaaaacacgattaaccctgtgcattgccgccgtcatctcttttgtcgcaggcgcaggcgcgagcgccgccgtggtgtt |
| | | caatatgatggcggggcaataggatgagtaacgcactagatcttcaggctagtaccacgtcagtacgttcgcaacgaaagtcctca |
| | | ttgaatattcaggagctcctgaataaaacgctgcctcacctggttcagaccataatcaggaatgagagattaaaaaacaccctact |
| | | tcaggttgatggtctcattatcggtaccggcgaggcggattttaccaaagggaatacccgctacgccttacatattgacgataaga |
| | | ccttccatctgctggacgtacccggcattgaaggcaatgagtcacgctatatcagccaggtgaaggaggctatcgccgaagcgcat |
| | | atggtagtgtacgttaacggtaccaacaaaaagcctgaaaccgccaccgccgaaaagatcaaatcatacctcgaatacggtacgca |
| | | ggtttatccgctggttaacgtgcgtggatatgccgacgcctatgaattcgaagaagatcgccacgatctgatgcagcaaggaggcg |
| | | caggagaagcgctgaagcaaaccgtcggggtactgcaaccggtgctgggctccgatgtgctgcttcccggtaactgcgttcagggg |
| | | ctgctggccttctgcgggctagcctatgacgatgcgacgcaaagcaccactatccacccctcgcgcgcgcacaacctcgccacgca |
| | | acagaaacgctatttccagcacttttcttctcgtcgggagatgcaggaatttagccagattgacgccattgcccgcgtcattcgcg |
| | | gtaaagtcgccacttttcgcgaagatattgttgaaagcaacaaaggcaaagtgcgagagtcactgggtcagtatctacaggtacta |
| | | aacacgcaactcaccaatcatcgcgcatttctaaagaaaacagagccggaatttgacaaatgctgcgtcgcctttgctaacgccat |
| | | tgcagcctttgaacgccgaatcatcaataaccgccgtaaccgctggaacgactttttcaatgatctgatggaaaaaagcgacgaca |
| | | ttgttgaagacgattttggtgataaagaggcgattgcccagcgtattagccagcagtttaaatcgcgtcgcgtcgaggtgaaaaaa |
| | | ttaatgctccaggacactgaggagggcgttaaggccttacaggagcagatgattcaagcggtggctcgtttgttgcaagatattaa |
| | | gcacattgagttccagcagcatgtcgatttcgcccacggcggtgaattcgaatttggtcgcgagatcgcgctgggttatgaccttg |
| | | ggttaagggatttcggctcaatggcctttaaaatcggcagctacgccttaagcggcgccacagtcggtagcgccttcccggtgatc |
| | | ggtacggccattggtgccgtagcaggcgctttagtcggcgtcgtcatgaccgttgtcggtttctttaccagcaaagcgtcgaaagt |
| | | tcgcaaagcgcaggggaaagtgcgcgacaagctagaaagcgccagagataaagcgctggacggtattgatgatgaggtccgtaacc |
| | | tggttgcggctatcgagaatgaactgaaaagcagcctgctgcaaaaagtgaatgccatgcatacggcattgcagcagccgatcgcc |
| | | attttcgaacagcaaatcacgcaagtcacccatttaaaaaatcaactcgagaacatgccttatggaacaattcaaacagttcagta |
| | | ttgagaagcaggctgccattaactcgctgctacagctgcgcggcatgctggaaacgctgggcgaaatggagatcgatgtcaacgac |
| | | gatctgcaaaaaatcgcgtcggccatcacagccgttgagtccgacgtgttgcgcattgccctgttgggggctttttcggacggtaa |
| | | aaccagcgttatcgccgcctggctcggcaaaatcatggaagatatgaatatctcgatggacgaatcttctgaccgtctgagcatct |
| | | ataagccggaaggattacccggagaatgtgagatcgtagataccccggggctgtttggtgataaagaacgagaaatagacggcaaa |
| | | caggtgatgtatgaagatctcaccaaacgttttatttccgaagcgcatctgcttttttacgttgtcgatgccactaatccgcttaa |
| | | agagagtcacagcgccatcgcaaaatgggtgctacgcgatctgaataagctgtcatcgaccatcttcatcatcaacaaaatggatg |
| | | aagtgactgatttaaccgatcaggcgctgtttgcagaacaggcggccatcaaaaaagagaacctaaagggcaagctacagcgcgcg |
| | | gcaaacctgaatgcgctagagcttgaacagcttaatattgtttgcattgcttcaaatccaaacggtcgtggccttcccttctggtt |
| | | caacaaacctgaacattacgaaagccgctcacgcatcaacgatctcaaaacagttgccgctgagattctgaaaaccaatgttcccg |
| | | aagtgctgctggcgaaaactggcatggatgtggtgaaagatatcgtcacccagcgtatcaccagcgcccagctgcatctcagcaaa |
| | | ctcagcacgttcgttgcgaaaaatgatgaagatacttcgcgttttacatgcgatatccagcaaagccgtaacgaggtcaaacgtct |
| | | ggctggcgaaatgtttgaagaacttagtttgctggaaaagcagctgatgagccagctacgcccgttggagctggatggcattcgcc |
| | | cctttatggacgacgaactgggctataacgatgagggcgtcggctttaaattacacctgcgtattaagcatattgtggatcgcttt |
| | | tttgcgcaatcctccgccgtcacgcagcgactgtcggacgatattactcgtcagcttaattccagcgagagcttcttaagcggagt |
| | | tggcgaaggggcatttaaatccctcggcggcgtgtttaaagggatttccaaaattagcccggagacgattaaaaccacgatttttg |
| | | ctgcacgcgataccattgggcaattaacgggctatgtctacacctttaaaccgtgggaagcgaccaaactggctggcggcatcgct |
| | | aagtgggctggtccggccggggccgcatttaccatcggctctgatctatgggatgcctataaagcgcatgaacgtgagcgagagct |
| | | ggaagaggcgaaaaatgagttgacccggatgatcaaagatccgttcagcgatatctatagcgtcttgagttcagatgaaaagacgt |
| | | tcgctttctttgccccccagattcaagagatggaaaaagtcatttgcgatctgacagaaaaaagcgacaccattcggaagagccag |
| | | caaaagctaagcatactccagcagaagctcgagcagtttaaccgttcgagcgagcagcaagtgtcctgatacacaaacggcagccc |
| | | gcaggccacgtttagttataaatcaaactaaacgtggccaggtgacatgccccccgttgattaacacacgttatcgtcgggtggaa |
| | | aggacaacctcctacgtccgcttcacagcggacactcaggtttaacagtccagtacgtttagcttacggataaatcattttatgat |
| | | gatgtggagaatgggggat (SEQ ID NO: 55) |
| 59 | pLG061 | tattttgcgtagctagaacgcaatcaaatctagcagtccgctttgttcggagttcggacattatgagttggcaagtaaagtagctt |
| | | gctaggaagccggatttgcacggtcggtataataagatgtaaccccttgccttcatttactcgaatgaacgtgcacattggatagg |
| | | aggaaaaggaatgcaattcattaccaacggccctgatattcctgatgagcttttgcaggcgcacgaggaagggcgcgttgtgttct |
| | | tctgtggagcaggcatttcctaccctgctggtttacctggtttcaaagggttggtagaactaatttaccagaggaacggaacaaca |
| | | ctttcagaaattgagcgtgaggttttcgagcgtgggcaatttgacggcacattagatttgctggaacggcgcttaccagggcagcg |
| | | tatagccgtccgacgcgcgttggaaaaagcccttaagccaaagctccgtcgtaggggcgctattgatactcaggcggcgctgttac |
| | | gtttagcccgtagccgcgagggtgcccttcgattggtcactaccaactttgaccgtctctttcatgtggcagctaaacgtacaggc |
| | | caggcttttcaggcctatgtagcgccgatgctgccaattccaaaaaacagccgctgggatggacttgtatacctgcatgggctgtt |
| | | accggaaaaggcggatgatactgccctgaatcgtctggttgttaccagcggtgactttggcttggcttatctcactgagcgttggg |
| | | cagctcgctttgtgagtgagttatttcgtaactatgtggtctgcttcgttggctacagcatcaacgacccggtactgcgctacatg |
| | | atggatgcgcttgcagcagatcggaggctcggtgaagtcacaccacaagtatgggcactgggggagtgtgagccggggcaggagca |
| | | ccggaaagccatcgagtgggaggccaaaggggtcactcctatcctttacaccgtaccggcgggctccactgatcattcagtgctgc |
| | | atcaaacgttgcacgcttgggcagatacttatcgagatggtatacagggcaaaaaggctatagtcgtcaaacatgctctggcccgc |
| | | ccgcaggacagcactcgtcaggacgatttcgttggtcggatgttgtgggccttgtcagataaatcaggtttaccagcaaaacgctt |
| | | tgcggaactcaatcctgcaccgccgctggattggttattgaaagctttctcggacgaacgatttaaatacagcgatctgccacgct |
| | | tttgtgtatctccgcatgtcgaaattgacccgaaactccgattcagtctggttcagcgtcctgcgccctatgagctggccccgcag |
| | | atgtcgctggtttctggatgtgtcagtgctagcaaatgggatgacgtaatgtcccatatagcccgttggctagttcgttatctggg |
| | | cgaccctaggttgatcatatggattgctgaacgcggcggacaaatacacgaccgttggatgtttctgattgagagcgaactagatc |
| | | gcttagcagcactgatgcgggagcgtaagacttctgagttagatgaaattctcttgcattcccccctggctattcctggtccacct |
| | | atgtctactttatggcggcttctgcttagtggtcgtgtgaaatcgccattgcagaacctggatttgtatcgttggcaaaaccgctt |
| | | aaagaatgaaggcttgacgactacattgcgcttggagttacgcgggttgctttctcccaaggttatgttgaggcggccgtttcgct |
| | | atagtgaagacgattcgagcagcactgatgaacccttgcgaatcaagcaattggtggattgggagctggtgctgactgctgattac |
| | | gtacgttcaaccctgttcgaccttgctgacgagtcatggaaatcgtccttgccatacctgttggaagattttcagcagttgttgcg |
| | | tgatgcactggacttgttgcgggagttgggagagtccgacgatcgtcacgaccgctcgcattgggatttgccgtccatcactccgc |
| | | actggcagaaccgggggttccgcgattgggtgagcctgattgaattacttcgggattcatggttagccgttcgagccaaagacagc |
| | | gatcaggcctcgcgcattgctcagaattggtttgagttgccatatcccaccttcaaacgtctggcactgtttgccgcaagccaaga |
| | | caactgcataccacctgagcggtgggttaattggttgttagaggacggttcatggtggttgtgggccacggatactcggcgagagg |
| | | tattcagactgtttgttttgcagggacgacatctgacaggaattgcacaagagcgtctggaaactgctatcttggcagggcctccg |
| | | cgcgagatgtacgaggataatttggaagcagacaggtggcattatttggtggctcattccgtctggttgtgtctagcgaagctcag |
| | | gggagcgggccttgttttgggagagtctgcggctacacgtttgacggaaatatccacagcatacccaaaatggcaactggcaacca |
| | | acgagcgtgatgaattctctcactggatgagcggaaccggtgatccaggcttcgaggagagtatagatgtcgacattgcgccccgt |
| | | aagtggcaggaattagtgcaatggctcgcaaagcctatgccagaaagactgcctttctatgaggacacttggagtgatgtttgccg |
| | | tacgcgcttttttcacagtctgtatgcgttacgtaaactatcacaagatgatgtgtggcctgttggtcggtggcgtgaagctctgc |
| | | agacttgggctgaaccagggatgattttgcgttcgtggcggtacgccgcaccgttggtgcttgacatgcctgacgcagtacttcag |
| | | gagatttcccacgctgtcacttggtggatggaggaggcttcgaagaccatcctctgccacgaggagattctactggccctttgtcg |
| | | tcgggttctgatgatagaaacaagcccagagtctagcaccattcgaaacggaattgagacctatgatcctgtttctacggcgatca |
| | | atcatcccattgggcatgtcacgcaatcactgatcaccctatggttcaaacagaacccgaatgacaatgatttgcttcctgttgaa |
| | | ttgaaaacacttttcaccaaattgtgtaatgtacagatagagctattccgccatggtcgggtgttgctggggtcgcggctgatcgc |
| | | attttttcgcgtagatcgaccttggaccgaacagtatctattgcccttgtttgcttggagtaatcccgtcgaagcaaaagctgtgt |
| | | gggaaggcttcctctggtcgccacgcctgtatgaaccgttgctgatagctttcaagtcagattttttggagagcgccaatcactat |
| | | tctgatcttggcgagcaccggcagcaattcgctattttcctgacttatgcagctctgggccctaccgagggatataccgtggagga |
| | | gttccgaacggcaattagtgctcttccacaagaaggtctggaggtagccgcgcaggcgttataccaggcacttgaaggtgcgggcg |
| | | atcagcgcgaggagtattggaaaaatcgtgtccagccattttggcaacaggtttggccaaagtcccgcaacttggccaccccacgc |
| | | atatccgaatcgttgactcgtatggtgattgctgcccgaggtgaatttccggcggctttggcagtggtgcaggactggctgcaacc |
| | | gctcgaacaccttagctacgacgttcgccttttgctagaatcagatatttgcagccgatatcctgcggacgctctatccctgctga |
| | | atgccgtgattgccgaacaacactgggggcctcgagagttggggcaatgcttgcttcaaattgttcaagctgctccacaactggag |
| | | caagatgttcgttatcagcgattaaatgaatattctcgaaggcgcagcgtgtgaaagtgacaggcgttggacagtgcgaactgtgg |
| | | agcctaacaaggtaaagacactctaactgataatgctgcgccgctcgtgcaatgcaatacagtttttatctagcggtgaattatgg |
| | | tgttaaaagttagcccctgacacagggtgggtagttggctctgtgtcattgatgggtattagttctgatatgagctaataccca |
| | | (SEQ ID NO: 56) |
| 60 | pLG062 | gtaagacaagggttgagcaggctactaatcgttacacaggctaacaaaggcatattaagacgatttgtagcgctgtaaccttgaaa |
| | | attatgtacaagcgccccgcattacgtcgttttaaaggccatcggattcaggcccgacgcggcttcacgcgattataaccgtgaaa |
| | | aatcccccccgcatagaacctgaattatccccgccgccgcgcagaactgacagcgcttcagaaccgttaaccctctcagaaatccc |
| | | gcttttttactgtaaaaaaccatgcataaggtgcatggttttgcatgcgtttcaccgacactgaatcccccgccagcgccagcagt |
| | | agcgtgccctgaggccgttaatgcacccgtattaaaagcgccctgttaagcgagcaggcggggcggggcgagcattgcgcgtcggt |
| | | gttaccaattctatatggacattgagcaattcaaatataataaaggttgggtatatttcgtcctcaacgatgtcaaaaactgcaaa |
| | | agcgtattataattcagatcattttcagaccacctattttaatcatgcatgcaaaatggaatatgtgatgacaaataaaaacaaaa |
| | | tcaaaccattattaaataatatatccgctcgcctttgggatggtcgtgcagctatattgataggagctgggttcagtcggaatgca |
| | | aagccattaacaagcaaggcaagaaagtttccaatgtggaacgacttaggtgacattttttatgaaagtgtttactgcaaaaaaaa |
| | | cgacaatagatattcaaatgtattgaagctaggagatgaagttcaggctgcatttggtagagcgacacttgataaattaatcatgg |
| | | atcatgttccagataaagaatatgaaccatccaaattacatgtttcccttctttccttgccgtggattgatgtttttacgactaat |
| | | tatgatacattacttgagcgagcaagtgttaatgtcgactccagaaaatatgacattgtccttaataaaaatgatttaatgaatgc |
| | | tgaaagaccaagaattataaaactgcatggtagcttcccatcagaaaggcccttcatagttacggaggaagattacagaaagtatc |
| | | ctttagaaaattctccttttgtgaataccgttcaacaatcattgattgagaatactctatgtctgataggattttcgggtgacgat |
| | | cctaacttcttaaattggattggttggataagagataatcttggcacagaaaattcacccaaaatatacttgatcggtcttttttc |
| | | atttaatgaagcacaacgtaagcttttagaaaaaagaaatatttccattgttgatttaagttttctaggtgattttggcaaggatc |
| | | attatctagcacaccaacgctttatccaattcttatacgaatcaaaaaatcgagacaacctaatagagtggccaatagaaaccaat |
| | | tatgacagaattgtttttaatgatggcattgaattaaaaactgagaaaattaaaaagtgtatcttagaatgggctcagtcaagaca |
| | | atcatacccgaactggcttattttgccggaatcaaacagaagtaatttatggcaaaacactatagattggttatctgttgctaatt |
| | | atgatgtcgcttgggatggttctgatgatcttgattttggatatgaaattacatggcgactaaataaagctttgctaccaattttc |
| | | aatgatacatcagaattcttatttaagttgattgaaaaatatgagatcaattacgtttcggggataaataataaaatcattgactt |
| | | tgatgaaaaatactctcatataaccctcagtttaatgagattctgtcgacaagaaaaccttattgataaatggaagaatctaaacg |
| | | atttattaattcaaaatcttgatcgattaacaccagaggtaaaatctgattattattatgaaaatatattattttcatacttcaat |
| | | ttaaacttcgatgaagccagaaacaaactctccaactgggaaacgaataaactcctcccccatcatgaaataaaaagagcaggatt |
| | | acttgccgaatttggaatgcttgatgaagcaatcaatcttcttgaagaaactttatctacgattcgaagaaacagtttgctttcat |
| | | ctagaaacattgactattccagtgaatctcaagaagcatatggaatctatattttgcgaatgtttaaacggagtttgcgtttagat |
| | | agcaaagatgacgattattcatctgagtataactcgcggttggctacattatcacaatatcgcagcgatcctgaaaacgaaataaa |
| | | atacctagaaattaaactagagtcactaccaggtaccttcaagaataccaatgacacggatttcgatcttaacaaaagaacggtga |
| | | ccacttatttaggaggaagcccaacagaagtgaggtcattagatgcttttagtttctttctactggcagaggaacttggcctccct |
| | | ttccacataccaggaatgaacatttttagtggaatagttgagaatgcagctcgacatatttatcaatactctccagagtgggctat |
| | | tttttcaatatttagaacatttaacaaggataaggccaagagtctattcaatcgaaatagaatttcgtctcttgagcgaaaaaagg |
| | | ttgaagatttatttgatggatactacaaaaaatatgagcaaattatcacaaaaaaaatagaagatagattaaacgataaacttgag |
| | | atagaaatttctacgctatcaatcattcctgaaattctttcccggctagttacaaaagtatcatttaataaaaagaaagacattat |
| | | tcaccttttgcttaaactgtttaactcggataattttcatcaatacatggagactaaagatctattaaagcgcactatttccaatt |
| | | tgagcgacttacaaaagatctcactaatagatattttcattgatttcccctccgcgcctcccaatacccaattacatatgggtcaa |
| | | agatacaacttccttactccatttgaatgtctattaggggttacaataacccccccaaaagaaaactctaaaaaaatcgcatctgc |
| | | aaaattaaaaaaagatataaacgatttaaaaagtgataatttagacttgaggaaagctgtatcacaaaagctcataacattatata |
| | | acctagaaatgcttaacaaatctgacacgactaaacttataaaaaacctttggtcaaagcgtgataactttggattcccaataggc |
| | | agtggttactataaatttttctttataaacaaccttaacccagataatgaaaatatagccgacaaattcatttctataattaaaac |
| | | atacaaatttcctgtgcaagaaggaaaaagagttagtattacaggtgggttagatgagtattgtactgaactcaatggagcgctac |
| | | accatataagtcttccagagaaaaccctatctgaaataatttcaaaaatacatgactggtatgtcaaggatcgggcctggcttgaa |
| | | aaaagagatgatttagccaaggagttcactcttagattcagaaatatcacaaatatcataacgacaattttagaacaccataagga |
| | | caaattacatgctgaatctataaatgaaatatcaagcctactagataaaatgaaagaagacaagatacctgtaaactcagcagtaa |
| | | caatgctttgtctgaaaaataaaagcacttacctcgagagaataaaagatatagagaatggactatatagctttaataaagatgat |
| | | gttattgaagctatcaactcaacttatgtctttattagaaacaatgaatttccactaaccatcattcaagctatcagcgataaaat |
| | | cgcatgggatagaaaccctcgccttcctgattgctacaatttaattgcatatataattaactcgtgtgaatttactcttccagatt |
| | | atttaatagagaaaatccttcgagggctggcatatcaaataaacattgatgatagagattttgttgataacaatgaatatttgaat |
| | | caccttgagaaaaaacttagtgcaacaaagctggctgcttctatgtttagaaaaaatgaaacactaggtattgaccaaccttctat |
| | | cattcaagagtggaaaaacatgtgcaactctagaaatgagttcgatgaaattaggaatgaatggaacaacaatatataaataaagg |
| | | aagaacacccaatttatattgggtgttctgttcacgaaacccttttaccataatcgaatggcaatataaattgagattgaaattta |
| | | ttctcatctaattaatcagcccaccattg (SEQ ID NO: 57) |
| 61 | pLG063 | actagctaagcaataagggcgatcggctctcccatagatcgaggccgaatgatgttagcaatgttcactcttggctggaatctgcc |
| | | agaaatcgaggtcatatggtctgctttgagtgaggagcgcaaatggataaagccctcatgagttctttttcaatgacctaactttt |
| | | gagaggcactgggttagatcatgtttcatgtttgcaatacaatatatatttaaacttaggtttataacttaaatgttagttcctga |
| | | tctaaaccagattattaatcactcctagagtgaaatgagttaagccaagagttgataaaattaacagttttttttacaatatctgg |
| | | atgtttgctagcgaacaggcatctaaaataactatgctgagctaaacttacaattcaaattgtaccgaggataaaatgcaagtaca |
| | | acatcatactgaaccaaacttgaagaatgagattgtggctttatttaaggcttctcaattgatacctttttttggcagtggattta |
| | | ctagagatattagagcaaaaaatggtaaagttcctgatgctattaaatttacggagttgattaggaatatagcggcagaaaaagaa |
| | | gggttaacacaaacagaaatagatgaaattctaagaatcagccagcttaaaaaagcgtttggacttctaaatatggaggaatatat |
| | | acccaaacgaaaatcgaaggcattattaggtaacattttttcagagtgtaaactctctgatcacgaaaagacaaaaataataaatt |
| | | tagattggcctcatattttcacgtttaatattgacgatgctatagaaaacgttaataggaaatacaaaattctgcatccaaatcga |
| | | gcagttcagagagaatttatatctgctaataagtgtctattcaaaattcatggcgatattactgaatttattaaatacgaagatca |
| | | aaatctgatatttacttggcgtgaatatgcacacagtatagaagaaaataaatccatgctatcctttttatctgaggaagccaaaa |
| | | actcagctttccttttcataggttgcagtcttgatggagagcttgatttaatgcatttatcaagaagcacaccatttaagaaatca |
| | | atttatttgaagaaaggatatttaaatttagaagaaaaaatagctctttcggagtacggcatcgaaaaagtaattacctttgacac |
| | | ttacgatcagatatatcaatggttaaataacacacttcagaatgttgagcgaaaatcccccacaagaagtttcgaactcgatgact |
| | | ccaagttaatgaaagaagaggctataaatttattcgctaatggaggccctgtaactaaaatagtggataataaaagaatcctgcga |
| | | aattctataactttttctcaacgagatgtctgtgatgatgcaattaaagcactacgtaatcatgactatatcctaattacaggtcg |
| | | acgtttcagcggaaaatctgtacttttatttcaaattattgaggcaaaaaaagaatataatgcctcttattactcttcgactgaca |
| | | cattcgatccttccattaaaaactcattgataaaattcgagaatcatatattcgttttcgactctaatttctttaatgcacaaagc |
| | | attgatgaaattttaaccacaagggtgcatcctagtaacaaagttgttttatgctcgagttttggtgacgcagagttatatagatt |
| | | caagttaaaggataaaaagatattacataccgaaattcagattaaaaataacttgattaatgaagaaggtaactatctcaatgata |
| | | agctttcttttgaggggctaccactttataaatcttcagaaacgttgttgaattttgcttatcgatactatagcgagtataaaaat |
| | | ttagactaagtggttctaatttatttaataagcaatttgatgaagattcaatgtttgttttgattttaattgcagcttttaataaa |
| | | gccacatatggtcatatcaacagtcacaataaatattttgatattcagaattttatttcgcaaaatgatagattatttgaattgga |
| | | gtcaactaacacagatccaagtggagttataatctgcaattcaccatcctggcttttaagagttatcagtgagtatattgataaga |
| | | atcctgcatcttataaaacagtatctgatttaataatatctcttgcgtcaaaaggatttcttgcagcatcaaggaaccttataagc |
| | | tttgataaactaaatgaacttgggaatggaaaaaatgtccataaatttatcaggggtatatataaggaaattgcacatacctatcg |
| | | tgaagatatgcactactggttacaaagggctaagtcagaattaatatcggcacacacaattgatgacctcgtcgaaggaatgagtt |
| | | atgcaagcaaagtaagactcgatagtgccgagtttaaaaatcaaacttattacagtgccacattagtattagcgcagttgtctgca |
| | | agggctctatctataaataatgataaaatatatgcgctgagcttctttgaaagtagcctagaatccatccggaattataataataa |
| | | ctcaaggcacataaacaaaatgatggataaaaatgatggtggctttagatatgcaatacaatatcttaaggataatccattaatag |
| | | aactccttcctcgtaaggacgaagttaatgaattaattaacttctatgagagtcgtaagaaataatcatccttaaattaataaatg |
| | | gcaagtaactcattcccttgtcatttattaaactcttaagagccttatcccgaaaagtattaatctgagctaataagattgttttt |
| | | cagctatgtcattattttattgccaatatatttacacttaagcattgacaggtagcggatagttatttttggcttgtaaataagcc |
| | | ttttaataatagaactgtaagacaatcgctctgattttttgaaatttatctcaatgttaaattcttccgcttttggcacaaacggg |
| | | ctagagcagacagatttaatgagataagggtatagatgaattctccatacccttgaacgattacttcccagttgatttgcttggtt |
| | | tcagtcctggggtattaccgggtgtatccttattatcacgtctgcgttgatcgggttttcctgttgattttgcaattggttttgga |
| | | ccaggtttaagccccataatcgtactccttagccatgtcagaggttattcctcagtgtggatataaggggagcggtaagaattatc |
| | | aagcttggatgggcggtgaaaaatgactacttgactattatgtgagcaatgtcagcttttgacatttagaggccagcccattactg |
| | | aagtaagccaaaaatgagtcgcgatgagccctcaacaatgagggccacctcggagattg (SEQ ID NO: 58) |
| 62 | pLG064 | gacagcttccagggtatcgtggacgcgtcatgcaaagagatggggatgagggattttaatattctaccccttgtaccccatgccag |
| | | tggtcgacctcataaatcattgattttaaaagcctcacttagggcgctcgctgccaccgatgccccacgatgcctgacgatcttca |
| | | acgactccccgcaaaagtccctatgcctcggaaaagccgccaaccccaacaacaccacctaacaacaagaaacaggacctcgtgcc |
| | | gagcttgttagcgcgactgactagccgtccgaaagcaaaaacaccgcgagccaaacaaggcaatttcttgcccccctaaggaacca |
| | | cctgaggattgaacaccagcgcagcttactgtatataaaaacagttaaagtcctgttctcaggctgcatctggatcacacagccgc |
| | | cgttactcggaaacacggcggattagcgcgcacgctcaggccctccagccctaacggaatatgaatatccagaaaatcaaacacat |
| | | atcagcctcacgcagcgcatagcgccctgccagaacacagcaggaagtcattgcgtttgcgttcctggcaatccatcattcacggt |
| | | tagggcccctataagacctgcagaagcagcgcgccatgggcagacccggcaaaagcccccaaacgggtgtggagaagctttatgga |
| | | gaaggaaatcccccacgaaggattcacaggctctagtaaagagccgctccagacgctccttccctttaatatcgatgaacccgggc |
| | | aggagcccatgaaaatccaagatttccccccactccccgcctccgaacagccgttgatgtttgcagacttgtttgcaggctgtggt |
| | | ggcctgtccctcggtctctcactttcaggcatgaacggcgtgtttgccatcgaacgcgacaagatggctttctcgaccctatccgc |
| | | caacttgcttgaagggcggaaggtgccggctccgcagttttcatggccctcatggctaggcaagaaagcctgggcaatcgacgagg |
| | | ttctcgaaaagcacccgattgagctcagtcagctaaagggcaagatccatgtcttggcaggaggaccaccctgccaaggtttcagc |
| | | tttgcaggaaaaaggaatgaatccgacccccgcaacaagctgttcgagaagtacgtcgaaatggtccaggccatccgaccatcggc |
| | | ccttgtcctggaaaatgtccctggaatgaaggtggcgcacgccacaaagaaatggaagcaactaggtatctcgatcaagccccagt |
| | | cctactacgacaagctggtagagagtctggacaggatcggataccacgtccagggcaatatcgtcgactcctctcgcttcggggta |
| | | cctcagaagcgcccacgcctgatagtaattgggctcagaaaggacctggcccagcacctcgaaggcggggtagcccgagcctttgt |
| | | gctgctagaggaagcccggctcaagcagctacaagagttcgaccttcccgaggccatccatgccgaggatgccatctcggatatgg |
| | | agataggtcacgcgggaacgaggccctgcaatgaccctgactcccctaggaaattcgaagagattgcctataccggccctcgaacg |
| | | gcgttccaaaggctcatgcatcgaggctgtgatggcaccatcgatagcttgcgcctcgccaggcacaagccagagataaaggctag |
| | | gttccaggcgatcatcgacgaccccaactgtgccaagggcgtacggatgaacgccgagatacgccaagcatatggactcaagaaac |
| | | accgcatctacccaatgcaggccagcgctccggctcccactatcacgacactgccggacgatgtcctccactacaaggagcccagg |
| | | atactgaccgttcgggagtctgctcgactgcagtcattcccggactggttccagttccgaggaaaattcaccactggcggtagcca |
| | | acggacgaaggagtgcccgcgctacacccaggtgggcaacgcggtaccaccttatttggcacgcgccgtcggcttggctatcaagg |
| | | caatgttggatgaggccgtgatgctcgccggccaacaggcagagcgagaacaagaagagaaaatgatagccatcgcttgaacacat |
| | | aggagtcgaggggaatggatagctcccaactggaaggggcgcaatacccggccgcgcttgtcgactgggccggccatcactcagga |
| | | ggcgtaaaaaggctgctggataaaaatagcggccagcctaacaagcagctgctacggacgaaccttttgtcccgtctccaggcctg |
| | | ggctaacaggcttcccaccgagacctcagctgtccccaggattgtcctgcttgtgggtggtcccgggaatgggaagacagaggcaa |
| | | tcgagtgcaccatccgctggctcgacgagagcctcggctgcgatggccggttggtcgaggaactctcgaaagccttccatccctca |
| | | accggctccgcagtcccccggctggccagggtagatgccggcagccttgccaagctagatagcagactgagcctcgacattgtcca |
| | | ggatgcctctgctaccgccgggcatgagggaagcaccgcccccgtccttcttatagaggagcttgccaggctactggatggacctc |
| | | cgacccaagcctatctctgctgtgtcaatcgtggtgtcctcgatgatgccctgatccacgcaatagacaacaatctggaacaagca |
| | | cgaactcttctcgaggcggttacccgggctgtaagcctggcgtacaacgcgccttcatgctggcccctcgagggtttcccatccat |
| | | tgcagtctggccgatggatgccgagtcgctcttggtaaagccggacgacgagcccgtagcccctgccgagatactcctaggccaag |
| | | ccactgctcccgatatgtggccagcgaaaggggaatgcccagcaggcgacaaatgccctttctgcgccagccaggccatcctcgcg |
| | | cgggatgagaacagggcatccttgctgaagatattgcgctggtatgagctcgccagtggcaagcgttggagtttccgggacctgtt |
| | | ctccctcacctcgtacttgctagcaggccaccatcctgtagtccacgatccctcagggactccccaccagtccactccttgccaat |
| | | gggctgcgaaccttgtcgacctcgaccaaaaggccctaacggcgaaaaggcatggcaagcagtcgctaactgccattttccacctg |
| | | tcgacttcgagctaccaacatgcgctcttccatcgctgggacaaggacgcagctacctcgctccgccgcgacctcaaggatcttgg |
| | | cctcgagaaggaactcgagatggaggaagggcgaaccctaatggggcttgtctatttcctttcggagcgcaaaagccactatctcc |
| | | cagcgaccatcgcccctctgctggaggggctggtcgaaacgctagatccagccttcgcaagcccagacggagaagttgcagtcagc |
| | | agtcgaaacacaatagtcctcggcgacttggatatgcgtttcagtcggtccctggccggaggtattgaattcgttcgtaagtacca |
| | | ggtgctatcgccaaacgagctcgatttactccggcgcctatccgcatcagacgccatgctttcgttaccgagcatacggcgcaaga |
| | | ggccggtggccgccagccgagtccagcacgtcctccgtgatttcgcatgtcgcctagtacgcagaagcatatgcacccggacggcc |
| | | atcgtggcggacgctcccattctcgaggcattccagcaggtcgtcgaggacagcgacaagcaccatcacctcttcaaggtggtaag |
| | | gcaagtaaaggaattgctgaacactgggaaggagttcgaggtgtcactaaccactacctttggccaaccactcccccctcgacaac |
| | | gccaggcaacgctggtcgtcccgcagagcccggtccggatgtccccccagaacaacaagggacgccctcacccaccgatttgctat |
| | | ctccatgtcggccaagggcaatcagtccagccagtcccactgacctacgaccttttcaaagccgtgaaggaactggaaagagggct |
| | | ctcacctgcatcccttccacgcacagtcgttgcactgctggacacgactaaggcccggctttccggcccgattgtccgcgaccatg |
| | | aactactcgatgatgcccggatccgcatcggcgcagatggcacggtggtcggccgctcgtggaatggttttgctgaaagccgggag |
| | | gacgacgtatgagccttgcggatttcaagcagaccccgtggagcaaatcacatccgaactaccagaagtcggccctggcaatcagc |
| | | cctgcccctgagtatgcgagctcggaagtcctgcttgcctcgctctaccgaaccataggcttcgcaacagccagcgagggcggcgt |
| | | gccgcaggccgggcgagatctagacaagcgtatccagaaactccgcgagaaacgccaatccccaccaacaggagcggtagtcggtg |
| | | tagaggcttggaatactgtgcttcacgggatcctggagagcccgaagcttcccaaccagtcgtccaagcgtttcctccaggtaacg |
| | | cccatcgtacccggggccgcactcttctccgggtctgcccgtctgagcagcaactcgtggcccgcaggcagcttgattcgccgcat |
| | | ggtctgcctgggatcgatggatggggagacggcgcaacgactttggcaacgcctcttcgctgcattgaacgtggacgacgaggacg |
| | | atgtcttcgcacgctggcttgaccaagagacatcggcgtggaacccgggagcaagcaactgggcactctcgccaatacccgcggac |
| | | gagatggtcacgttggagacggcagatttcctggggatcccctttctccccgcccggcgatttaccaaggacctacaggccatcat |
| | | gcaggccaagggttcaatgacccgccggcagtggactagccttctcgaggcattgcttcgcctggcagccgcatcccacgtgacgt |
| | | ggctgtgcgacgtccacgccaggacttggagctgcctgtgggccgcactaacggatggcattgctccttccagtgaactggaagca |
| | | agacgggcgctgttcccggaagccccgcagtacatgacgtacgggggaaaagccctccaaggcatcaaggacaaggtgtctagcta |
| | | cctaaatgcccggctgggaatcaatgccctcctctggtctctggcgcagataggagctccctattctggcaacctctcctcgagcg |
| | | ccggaattgctgcactttgccagcatattcgtcagcacaaggccgagcttactcgcctaggcacgcttgagacgattgccgatgtg |
| | | cgcgagcaagaagcccgtgcgcttctttgcaagaaaggcatcggctctaacctgctggagtttgcgcggcacgtccttgggcaacg |
| | | ccaggctgcagtcccattgctgagggggtacgaccagggatacatcctgaagaagaaaggcagcagcccgtccagcccatgggttg |
| | | tctccctcggccccgtcgccgtgcttgccttggtccactgcgcccttgcaggaatgggcggtccccgctcggtccaccggcttgga |
| | | cagcacctagaggcttatggcatggccgtggacaagcatgacattggcaggaacgacctgggccaccagttgcgaatgctcggcct |
| | | agtgctagatagccccgatgccgaaagtggcatgctgctactccccccgttccccataaaccaagccagccagggcccggaacatg |
| | | aatagacttgcacactggcttgccgccactgtccacgagaaagtcaggggctcgacacaagggttcggaggtaccagcctagaata |
| | | tcggcttatcttccgcggcccacccctcgagctactcgaaccggcctacgacgagctggcccgcaacggagggatccaggtgccaa |
| | | gcggggcagacggaggactggtgaccctgccggtactgctccagtatccagccggccagctgcagggacccaggccacgcatcgga |
| | | gcatccggtaagtgtgacaacgaccacttgcttgatatacgcaacgaccctgccaaccctagctttattgccctggtcccgccggg |
| | | actgcacaacaacctctcgatcgagtcaaccaccgacgaattcggattgggggcagccaccagcacggggcatgcatccttcgaac |
| | | aatggtgggaggatggctttgtccagcaagcagtcaacgaggcgttgatcgctgccggcataacggacgcccagagggatgacgcc |
| | | aggggcctggtccgcgcaaccgcagcctcggtcgacgaggtggatccagacaagggaggtcatcgcgcggcctggcgcctactctc |
| | | gcgcatctactcgatagcaaacgtgaatcaagggttgcctgcaggaacagcgctatcactggcatgtggtcttcccccaatgaagg |
| | | agggaggaatttccgccaagactcagctttcggtcctgggaaaaatcgccgacgagcttgcggacggtttcaagactggcatcgag |
| | | cgcctggcacaaggcgtccaacaaggggttgcgcaagcgctgcgcgaactgctttcccatctccactcgaattgcgacgtacctac |
| | | ggccttcgagcgtgccacagcggctttctacctgcccagtgccgatattgaactggcgcctcctccatcctggtggaccacgctca |
| | | ccaccgagcagtggacggaactacttgccgacgagcctgacgaggtcgtcggcgagctaacgatccggtgtaccaatagtttgatc |
| | | cctatggggaaaggcttgccggccgtagtacgggacaaagtcgagctattgatttccacaagcgaagagagccaaccaaaggagct |
| | | cctgttgacaggcggatcctacggcaaggttccgacgtcattgccagcgggccctaatgggactaccagccacattgacctatttc |
| | | cctcctcccacaaagcgccaatgagctacaaggtttccgcggacggctgcaagcctgcgagcgtccgggtcatctccctcgcgagc |
| | | tggaagcccggaatactcgttacctgcaggcttgcgacaaagctctcgccaccgaggaagccccgcaagaactcagctgcgatgga |
| | | ctgggaaacatccctgtcgctgccgggctccggtcgttatgagctccagctccaccttgctccgggggcgagcattggaaaggtag |
| | | aaggcttgccggacgatgccaccgaattcgaggagcagcgggagacaatcgaaccacggcaagttggggaatacgagtatctaata |
| | | gaggtcgaggctgatggcaagtaccagctggacatcgcctttactgaagccggcgagcaagttccgaaggtctgccgggtatacct |
| | | gacctgcgaagaggcaaaggaggaaggttgcaggagcgaattcgagcggctcatcaagctcaaccgacggcatctcgagaagttcg |
| | | ataccaaggctgttgtccatcttgaccggaacgcacgctcctccagcctgcagtcgtgggtgctggaggatcagaacgtatccaat |
| | | tccttcaggccactggtgatcgcggacgactatgcgtcccggtgggcccctcctgactgggacgccccgcacggccctgtactctc |
| | | gaacgggcgtttccttcatgacccccgccccgaggccacgagcttccaacctcccaagggcttcatcgaggctcggcaggggatcg |
| | | cccggtacatacgtggtagcgacgaccaatcggggctccttgagtcagcgccgcttggtgcctggctatccgaagaccctgggttc |
| | | cgctcccttgtcgaggactaccttggagcgttcatgtcttggctggacgccgacccgggtatcgcctgctggatcgacaccattgc |
| | | cgtctgctccctggagccggatggtcgtaccctgggaaggatcccagacgccatcatcctttcccccctgcacccattgcgcctcg |
| | | catggcactgcttcgcccagaaagtactccgtgacgaggccgagggcgaagccccgtgcccggcagcaagcatcctcgatccggac |
| | | tgcgtccccgatctadgaccatctcgctgcaggcaccgggaggagtggatcaggtcgacttcctttccgtcgaatgcagctccgac |
| | | tactggtccgtgctttggaacggatcccggctgggacaaatacccgatcgcgctcgccgggccccgttcgacagtagcttcgggct |
| | | ggcagttggagggatatcgagcgggttcagccccgcccaggtctcacgagcactcgacgacgtcaccgacctcctggcagccaagc |
| | | ctatcgtcagcctggtagtgtccagcgcaggtggcaccacggatgcatgcaacgaagggttggccacctggtgcaccaagcgattc |
| | | ggcaacggggaccatgacaccccgcggcacggtgtcgggccaaggattgtggaggtattcgataccaggcaggctggccggcccga |
| | | ccaggcgacgatcgccaacctctccgaggacacaggcaaccacgtccgctggtatgacaagcaaccaactgggtccaagccagacc |
| | | tgggcatcattgcccaactagattcggcccaacccgaatccaaggaggtcggaatgctttcgccgatgggaaccggcggactgatc |
| | | aggcaccgcgtcaggcgccaactccaagcctccttcctaagtgaatcccggcagggcctgcagatgccaccctccggcgaaccgtt |
| | | cgcagataaggtttccgcatgcatgctcatgatggaaaggctcagggacggcaaggtcggcctgcagttctcccctaatgtccatg |
| | | cagtgtccagcatgctcgaggaaaacagcgctgggttcgtcgctgtatcgtcgtcagcaatcgaccccgcctgcttcctcggaggc |
| | | tggatacaagggacgtatctatgggactacgacctcccctcgtactcgcatcgcgcaggcgacacaagcggctactacctgttatc |
| | | acaggtcaagcaggctgatcgcgatgcgctacggcgagtcttgaagccccttccgggatgcgaggatctggacgatgatcaggtcg |
| | | agcaaatcctcctcgaggttgcgcggagggggattcctacggtgcgaggcctctccggggacgatacgggggcgacgggcgacctt |
| | | ggcctgttcctcgctgtccggctcctacaggatcagttccgtgtgacaggcaacaaggaaagcctgctgccggtgcttgccggatc |
| | | accggaggactcgacgatagcaataatcatccccgtcgaccccttccggggttacctttccgatcttgcccgctcccttggcaagg |
| | | agcgcaaggatacctccctgtcgcgtcccgatctgctggtagtgggcgtgcgcgcatgcagcgacaagatccacctgcaccttacg |
| | | cccatagaggtcaagtgcaggcaaggagtagtcttcggtgcaggcgaatcaaccgaggcactctcccaagccaaggccctgtcgtc |
| | | attgcttcgtgccatcgaggaacgtgcaggtagttctctggcatggcgccttgccttccagcacctgttgctctcaatggttggct |
| | | ttggcctgcgagtctacagccagcatcaggcagtaggtgggcatgccggccgctgggctagctaccatgaacgtatcgctgcagcc |
| | | atactcagcccaaccccgccgatcagcatcgatgagaaggggcggctgatcgtggtggacgcgtcgctccagagcagcccgcatga |
| | | tcgcgatggcgacaagtacacagagaccattgtcatttccagccgagatgccggtcgtatcatcgttgggaatgacgcacagtcct |
| | | tctatgatggcgtacgtgcaaaggtcgacgactgggggctgctaccctgccaggcaagtgcggccggcaccccaatcgtgcagccc |
| | | gacatcactcccccggacgatgtccagacgggcgaccccatagtagtcccagcagaagatatccccggggcatccaccagtctggt |
| | | cgatcagacatctaccggcgtagcggaaccaggggcaagccctgcccccccaactgacgagccagggacagggatcattctctctg |
| | | ttggcaagactgtggatggtttcgagcctcgatcactatccctgaacatatccgacacccggctcaaccagttgaacattggtgtc |
| | | gttggcgacctcgggacaggcaagacccagttcctcaaatcgttaatcctgcagatatccagggcccgcgaggccaaccgcggaat |
| | | cacgccaaggttcctgatcttcgactacaagcgcgactacagcagccaggactttgtcgaggccacgggcgccaaggtggtgaaac |
| | | cctatcgcctgcccctgaatctcttcgacaccacggggatgggggagtcctccgcaccatggctggacaggtttcgcttcttcgcc |
| | | gacgtactcgacaaggtgtattccggcatcggccccgtgcagcgggacaaacttaagggtgcagtccgcagcgcctacgaggtggc |
| | | tggtgggcaaggccgccagccaacgatctacgatatccatgccgagtaccgagagctgctcgcagggaagtcggactcgccgatgg |
| | | ctatcatcgacgacctagtggacatggaggtcttcgcgcgctcaggggaaacgaagccgttcgacgagttcctggatggagtcgtg |
| | | gtgatatccctcgattccatggggcaggacgacaggagcaagaacctgctcgtcgccatcatgctgaatatgttctacgagaacat |
| | | gctacgcacgccgaagcgccccttccttggcacgtccccacagctccgggccatcgactcgtacctattggtggacgaagcggaca |
| | | acatcatgcgctatgagttcgacgtgctccgcaagttgctactgcagggccgcgagttcgggacgggcgtcatccttgcctcgcag |
| | | tacctgcggcatttcaaggcaggggcaaccgactaccgggaaccattgctgacctggttcatccacaaggtacccaacgcaacacc |
| | | cgcggagcttggagtactcggcttcacctcggacctggcagagctatcagagcgagtgaagacccttcccaaccaccactgtctct |
| | | acaagtcattcgacgtggctggagaggtcatacggggactgcctttcttcgaactcaccaaccaagcctgaccaacgcccggcctg |
| | | cgaatacaggccgggcaaggaggctcctaatgacagacttcctttctcccgcagaacgctcggacaggatgtcacgtatccggggc |
| | | aaggacacgcagcccgagctagcattacgcaaggtccttcaccggctcggactccgataccgattgcatggcgcggggctactagg |
| | | caagccagatctcgtgttcccgcgatacaggaccgtggtattcgtgcatgggtgcttctggcataggcacaagggatgcaatatcg |
| | | ccacgatccctaagagcaacacacccttttggctggagaaattcgaaaagaatgtcgtacgtgacgcgcgagtagcaacagatttg |
| | | caggccttgggatggacggtacttgtcgtatgggagtgtgaactgacatctgccaaaaaagcccagaagactggcgaacgcctata |
| | | tgaggttatccgtagtcgtagccacggaaagtatcggtaatcgactgaagcagccctgcggcctgtagtggtctactgatcccgga |
| | | caccgatttaggcgaaaatcctcgccgtgagagaggtgtccg (SEQ ID NO: 59) |
| 63 | pLG065 | cgaacggagcaggtagatccgcgctaactgacttgcccaatctggctgcattcgtccaacgctaggcggcttcgcaggaaaagcga |
| | | aacggagggagattctacgcgcacctttgtgcagacctgaggctccaccagacctgagagcccggcacgattgactgatcatagga |
| | | gtaaggccaagaagcgacttgatgcgcttgtaaggtaaattctcagcgaatcgaagtaatgacaccgaaacacgtgcggtcgacaa |
| | | ccgtgtaagattgctgataaaaagagcaggacgtcacaagaaatgaacttggaagtagtgccggcgagccggactttcatcgacct |
| | | cttctcgggatgcggaggtttgtcgctgggactttgccaggctggatggaaaggactcttcgccatcgagaaggccacggatgcgt |
| | | tcgagactttccgggagaacttccttggtgagaactcccgctttgcctttgattggcccagctggttggagcagcgcgcacactcc |
| | | atcgatgacgttttggcactgcgcggtctacatttgtcgaaaatgcggggtgaagtcgacctcatcgcgggtggtccgccatgtca |
| | | aggattctcgttcgcgggcaagcgaaacgcgaaggatccccgtaaccagctctcccagcggtacgtcgatttcgtcgagcgactcc |
| | | agccgaagtccctagttctggagaacgttcccggcatgaacgtcgcccataagtatgagcacgggaagagtcgcaagacttactac |
| | | gaaaagcttctgcattcgctttcaatagccggctacgtggtgtcggggcgtgtcttggacgcggctgacttcggcgtcccgcagcg |
| | | ccgcactcgactaattgccgttgggattcggtcggatatcgcggataagcttgcatgcgcggctagctcgactcccgcagacgtgc |
| | | tcgagggcatcttcgatgcaatcaatcaggcaggcaagcgtcagctcgtccgatatggccagggcgcccatgtcacggttcgggac |
| | | gcgatctctgatctcgcgattgggccggccgatcacgagaacaccgaagactacgtgggaagcgagcgatgtgcaggctacaggca |
| | | ggtcaggtaccaggggccgaacacgccttaccagatcgccatggcttctggggtcaccccatccgaaatggacagcatgcgacttg |
| | | cccgtcatcgtcctgatgtagaaaagcgcttcaaggcgatccttgaaacttgcccgcgaggggtcaacttgagcgccgagttgagg |
| | | gcgcagcatagaatgctgaagcataggacggtgccgatgcatcccgaaaagccggcgccaaccctgactaccctgccggatgacgt |
| | | cctgcactaccgagacccgaggatcctgacggtccgggagtacgcccgaattcagtctttcccggactggttccgtttcaagggca |
| | | aatacaccacgggcggggcgtcccgtcgtcatgagtgcccgcggtacacgcaggttggcaatgcggtcccgccgctgctcgggcag |
| | | gccattggctcaggattaatggcgtgcctctctttgagttcaacgcgagtgataagggccagtgcgcccagtctcgcgatggccga |
| | | gaaaaaggcttttgccgtatagcaattagtcagctgcaagaatcgaacaggtggatagacgatgacgaaataccccgatggattgc |
| | | ttgattggtcgggcaatcgggctggaggagtcaagaaactcttctacggcggcagcggccgccccgtcgggaaggtgatagagact |
| | | cctctactcacccgtctctgggaatggtcggatagcgtcgtccagttcgagccgggcattccgcgggcggtgttgctgttgggagg |
| | | gccgggaaacggcaagacagaggcaattgagcagacgcttcgccgaattgactcaaggcttgcgctgagcggagcgctcatcgaca |
| | | agcttgcggctgtcttcgagtccaaggatggagtccccccaggacgccttgtggaggtggatcttggggcgctttcaggggggcgc |
| | | tcgagcgggacaatctcgattgtccaagacgcctcggaggggaatccgggctctcctgatcttccggcgcaattgctctgcaacga |
| | | cctagcaggactcgtcgaagacaacgtgtcaaagcgcatctatttagcgtgcataaatcgcggcgtcctagatgatgccctgatac |
| | | ttgcgacggaaagaggtgacacagaaattggtgctttgctgaagcaaatcatccggtcggtgtcgatggcggcccatggcgtctca |
| | | tgctggcctctgcagggatatccgggcatcgcagtctggccaatggatgtggagaccttggtcgcaggcgtccagggtcaaccttc |
| | | acccgcggagcaggttcttcatattgcggccaatgccgaccattggcctgatttcggggcatgcgaagcgggtcagtattgcccgt |
| | | tttgcacaagtcgcaggctcctttccggcgagccccatgcgggatctctcgccaagctgctccgatggtatgagctggcgagcgga |
| | | aagcgctggaacttcagggacctgttttcccttgtcgcccacctgttggctggaacccctagcaatgccgatgcgtccggttattc |
| | | gccctgcaaatgggcggcaaaacaactgaatccccccggcggcgacccgcgcaaggccgatgtactccgaaagcgcggagtctttc |
| | | ggttgctggcttcccaataccaacacgcgctctttggcgactggccaatcgagcatgcgtcgggtctccgaagagacatcgccgac |
| | | ctagggcttggtgatttcccggcgcttgtggctatccagcagttcctggcgctggataagcggcgggagtcgacggcaaccctccg |
| | | tgcccagctctccggcatgtcatccgtattggatccagcaaaggcaagccccaccttcgaggttagggtaagcgctaatactgtta |
| | | ttcgttacgaagacttggataggcggttcagcctgtccatccaaggaggcagagagtacctccaagaatatcagtgcctctcggag |
| | | atcgagatttcagcactcaaggtccttgaggaggccgacaataagttgtctgatcacttagtcaggcgatctcggccggcgacagc |
| | | aattcgagtccaggcgcttctgagggccatcgcgtgcaggctggcaaggaggtcgattggcgtcaggtgttgtgtcacaaaggatg |
| | | ccgacgtcctcgaggagttccaccgcgtcaccaatggcgattcgtcggcgctgcagcaggcgatcaggcaggtcgaggcacttctc |
| | | aacgtcaatcgccggttcgttgtttgtctcaacaacacctttggtgagccgctgcctcccccagagcggcgcgcgatgcttaccac |
| | | ggacattcagcgcgttaagccggtgcccgccttggagggtgttgagcggccgagatcgccgatgcccttcctgagggtcggcgcac |
| | | aaggcaacgccaggcccatagccctgaccttcgatctcttcaaggcgacgaaatcccttaggcgtggcatggtcgcgtcgtcactt |
| | | ccgaggtcggtggtcgcgcttctcgatacgacccgagctggtcttgcgggagcgatcgtgcgagacgaagacgctctggaaggtgc |
| | | ggagatccggatcggaatcagggatgaggtcatagtgcggacctttggaagtttcgtcatccgccaggagggtgcttgatgtccat |
| | | gcaggagtttctcgcttcaccatggaagaaagaagcctcgcaccgagccttcaacgaatcctcttttggtatgaggtctgccccgg |
| | | agttcgcaactggcgaggtcgtcctgtcttcgctctaccgcgccgtcggctttgacggggtttccgaggagaaagtgccctcgctt |
| | | ggcaatgatttcaggaaggcgctggacaaggaacgcagaaagcagaacgcagctggtggtctgagcccagaagcctggcgcacggt |
| | | cgtggatcgtgtcgtgcaaagtcctaaggttgcgcagcaatcctccaagcgattcctatcgctgtccccggtcgttcccgacgcgg |
| | | ccatctactcgggcgccgcgcgccttggaggaaactcctggaacccggggcggctgatcaagcaaatggtcggaatcgggtcggag |
| | | accatggagggcgcggaaacgctttggggcgaactctacgatgctttgtccgtgacggaagcggatgatgtctgggcaagatggct |
| | | ccaaacagaatttagtcccaggcgcccagagcaaatagcgtgggccccaagaccgatggatcaaccagatttgcttccgcaatccg |
| | | atagacggggagtttcctatcccgctcggcagttcgtggtggacctgcgaggaatcttggatgcgaagtccgccatgacgcggcgg |
| | | cagtggatcacactgctcgaggcgctacttcgaattggatcggtcagccatgtgctgtggctgtgcgacgtcaatgaccgcttgtg |
| | | gcgtgcgatgcgtgcggcgctcgagggcgaggcgagtggcgtgcccgccgatgccgccgccataagaaccgacattctggccgtca |
| | | ggcggcggacgctctcgttcgggaatcccgctgtcccagcgattcgggacctggcctctcgatacctatccgcacgcctgggaatc |
| | | aactgtgtcctttggacgctggacgaacttggcgtgggctcaagtcgactttgttcgtccgaagaaatccttgacttcatcaagag |
| | | cgttcaggccaacgcaggggggctcaaggcccgtggcgtcatggatgccttccattccctgcaagacaaggaagtcaggaccattg |
| | | gctgtaagaaaggagtcggagcaaaccttctggaattcagccagtacacgcttggacagaggcagacgatggaccaggcactccgc |
| | | gggtacgaccagagctatttcctcaggaagaacggggatgccaggaacgcgccatgggttctatctctagggcccgctgccgtact |
| | | tgcgatggtccactcgtgcctacatgcggtggatggaccgcgatcgatacaaaggctttcatcccatctcgggagctacggcatcg |
| | | agtttgatctccacggcgtcaacgatagcgtccttggaaagcaactccgaatgctcggactcgtactggatagcccggatgccgag |
| | | agcggtatgctccttgtgcccccgttcgtagcctgaggaaggaggcaatgatgagcacgctagccaagggaattgcaagctgggtc |
| | | gaaaaagccatggcgcgtgagatcgcgacgctggtggccgggaatatggagtgtcgcgcagtcttctgcggcccgccaaagcacat |
| | | cctgaatcaagtatttgggcatcttatccacggtcgatcgctgatcgaagcgacaagggccgatggtcaggcggttcagtatcccg |
| | | tgatccttcaggtcgaccgcctccctacagggtttcccatcggctccgccacacagtcgggatgccttcagttccatggactcgct |
| | | gccgtcaggaacgacaggaatggtgttttcctagttcttgtcgagcccggtgctcaagcgagcgatacgcatgaatcaactcgaac |
| | | ttcgcttggactcgagccatcggtaaacgagggcggtgcctcgatcattgcctggtggtctgatccattcattcagtcgcttgttg |
| | | attctgccctctcagaactctccggtcgcgacgccgcggctgccaaggatctactaaaggaggcgatgatcgccgccgacgcggca |
| | | gatcagcacgaagtagcgagagttggagcctggcgcgtcatcgaacggttgtgggagctaaaagaacgcggcttgtctcttgacca |
| | | actcgttagcttggccgccggattcccgccctctagcgacggaagtattgaaccgagatccaagaccgccatcctttcagccatcg |
| | | tggacaggatcgaagccgagaacttcggtggcttactgtcgtcccttctgcaaaaagccagggacgatatcgaaaaagaacacatc |
| | | accgcgtgcctctcgaatatgaggggcaggtgcgatgtggttactgcggttcggcgatgtgcgccatatgcgtacatgccttcgga |
| | | cgccatcgctggcgaagtctggtggaagtcgctcactgtcgagcgctgggaagagttgctcgatgatggcgctctacccgatgcgg |
| | | gcggcgacatcattattcagtgtgccaatccgatgatttcgcaccttaagggcatggttcccgtcgtcaagggatccgtgcaactt |
| | | aggatcgaggttccagagaagtacgtgggcaggcggttggaggttatccgcgaggtcccgggtgcgaaggcggcgacgaaggtttg |
| | | gacagttgacgcggaacgcatgatccacgtcgaggacgacgagatccccccccacaagagtccgatgaagtactcggcaagcctcg |
| | | aaggatcagccggaaagaaggcgagcgttcgaattgtctcaatggatggctggctccctggggtggttgcctctgcgacgacggcg |
| | | acaaaaggttccctcccgaaacgctcaaaagcagcgaagttagaggcgtcgctgtctctctccgggcaggggaggcactaccttga |
| | | catctacttaaggccgggcgtcgagctcgcgtcaatgctcgccaccggtagtgacgaggaaggaaatccagacccgtccatcacgg |
| | | cgccaatcggcatggtcgcggagggcgagttcggggtcgaaatcgaaatcgaaggggaatgcttcttcgacatcacgctcagggtt |
| | | ccggaggttgcggatgatcaggtcatccggatcgaattgtcggcggagcaatcaagcccggaagagtgctcaagccacttcgaatt |
| | | gcagctccttaagaactctagcggtcggaagcccagcgcggtccacgttaatgctcagctaagaagtgcgcagcttcaaggttgga |
| | | tgctggagcaggggcgcgctggtcgctcctattatcccttcgttatggccgcggactatgccgccgactggcacaggcgggactgg |
| | | actggcgcagatgacacgatcttctcgaaggctagcttcctgtgcgatccccggccctcgccggaagaaatggcgccgccgcaggc |
| | | tttcatagatgccagagccgcactggccgccaggatcaggggtggtgacggaaatggcttggtcgaaggtgtgccgctcggtgagt |
| | | ggatggcaacggatcccgatttcgccggggaaatagacgtctacttgaaatcctacatgcactggcttgcgagcgatccagatggg |
| | | gcggtttggtgtgacgtagggttggtcgcgcggctcgagcctaacggacttaccttggtgcaagagccggatgcggtgatagttag |
| | | cccgatgcatccggtaagacttgcttggcactgtgtggcccagcgagccatgttccttgccgcacgaaagagaccttgtccagccg |
| | | ccagcatcctcgatccggattgtgtgcccgatgcgatcactctcccactgagaaacgccatgggtggcaagaccaacgccactttt |
| | | ttctcggtcgaatgcagttcggactactggtcgattctttggaacgcggggcgcttggaagccctttcttcacatggggcgacagc |
| | | cccgcttgaccgggagtttggcctactcgtcggcggaatctccggtgggtttagtgtttcgcaggtgcacaaagcgctcgaggaca |
| | | tctgttcgatgctggtggcgaagccggtcgtcggcgtcctggtgtccagtaccgcgagccagaacaatgcgtgcaatgaaggtctg |
| | | ctttcctggggcaggaagtacttcggcggcggggatagggcggcaggcttggacgcctgggtcggggccagcgaggtcaggatcta |
| | | cgacgacagaccggaagatgcccggcctgatgatgcggagatttcaaatctggccgaggatacggcgaacgccgtgcactggtatt |
| | | ccggcacggtggccggcgaggctcccgatctagcgatcatcgcccagcttgagacctccaatcccggtgcactcccaaccaaacta |
| | | aattctccgttgggcttcggtgggctcgtgaggacccgaattcgggagccttccagcatggcggggggtcaactgctccgtgagtc |
| | | gcgcatgtctggtcccgcggcgcccactggcgacgggctggccgacgctgtagcaagtgccatctcgtcgctcgagaacatctcgg |
| | | agcaacgccttggttacgtattcgcccctagcattcatgtgatcaagggggcgctggagagcgcggaatttgccgcagtttcctct |
| | | tcgagcgttgacccggcctgctttctcggaagttggttggagggcacctatctttgggactacgagctcccgtcgtactcaggtcg |
| | | tgccggagacagcaatggctactacttgttgtcacggatcaaggatctcgacctcgaaaccctgagaagcgtggtcaagaggttcc |
| | | ccggttgcgaggagatgccggaagccgtgcttgctggaatagtcgaggaggtcgcacggcgtggtattccaaccgtcaggggcctc |
| | | gccgcaggtgattctggcgcgacgggtgatttggggctactcgtggccacgaggctgcttcaggatagcttccgggcggccgaatc |
| | | aggcgctggtctcctgacgccttggcgcagggagggagacatcgaagagcttgctctcgtcattccggtggatccattccagggct |
| | | atcttgacgatctcgcgaaggcgctaaagcgccctacgctccaccgcccagacctattggtcgcgacggtgcgaatcagtgacctg |
| | | ggagttcaggtccgactgactcccatcgaggtcaagaaccggggtgctggagcggcgatgccgcaatccgatcgagaagccgcgct |
| | | tgcccaggcacgctcgctggcatccctgctagatgcaatgctggcaacgtattctgaggatcaagagatggttctctggcggattg |
| | | cgcaccagaacctcttgacctcgatgatcgggtacgcattccgtgtttacagccaacgtctggcagcccaaggcaagtcgggagac |
| | | tggtcgcgcctgcacgcacgagtcatggaagcaatcctgagctcccaggccgatgtgcgggtggattcgagaggccgcctgatcgt |
| | | gatcgatggctctagccaaagtggtccgagggatacagatggagatggtttccacgagactatcgagctctcgcacaaggatgctg |
| | | cgcttttcatccgtggcgagcacgatgcgctctgcacggccatgaagcagaagctaggtggctgggaaatgttccctgaagggagg |
| | | gatgccggactctccaatcaatcgccgcccgtggcccatgagactgcgcccttggtggatggcggcgttgaggtgccgtcccttca |
| | | cgcgctccaagcaacggcggggcccgagggcagctcgctgccgtcttcgggagtcgaagccatgggcgcgtcgcagccggcctccc |
| | | cgggagccatcgacgtggatggcggcatggcccagtccgggctgatcattcgggtcggtgaaacgatcgatgggtttgagagccaa |
| | | attcggcggctgaatcttggcaacacggccctgaaccaaatgaacatgggagtcgtcggcgatctggggaccggtaagacgcagct |
| | | gctccagtctctggtttaccagatagccaaggggaaagatggaaatagaggtattgagccgagcgtcctcatcttcgactacaaaa |
| | | aggattactcttcgaaggagttcgttgatgcggtagctgccagggtcattagccctcatcaccttcctctcaacttgttcgatgtt |
| | | tcaactgcatcgcagtccatcaatccaaagctcgagcgctacaagttcttctccgacgttctggacaagatctattcagggatcgg |
| | | gccgaagcagcgagaccgccttaagaactccgtcaaggacgcatatgtgcaagccgccgaagggcagtatccaacgatttacgacg |
| | | tccatcgaaattacgtagaagcacttgatggaggcgcggactccctgtcgggaatcctaggcgacctcgtagacatggagctcttc |
| | | acgccggatccaagtgtcgttgtttcgtcggccgaattcctgcgcggagtggtcgtgatatcgctaaatgaacttggttccgatga |
| | | ccggaccaagaacatgctcgtggccatcatgctcaacgtcttctacgagcacatgctgcggatacagaagcggcctttccttgggg |
| | | agaaccgcaatatgcgtgttgtcgactccatgctgctcgttgacgaggccgacaacatcatgaagtatgaattcgacgtcctgcgt |
| | | cgggtcctcctgcagggacgtgagtttggcgtcggggtgatcctcgcttcgcagtacttgagtcacttcaaggcaggtgcgacgga |
| | | ctaccgggagcctttgctttcctggttcatacacaaggtcccgaacgttcgtccgcaggagctttcggcgcttggctttagtgatg |
| | | cggtgggattgccgcaattggcggagcgtatccgtagccttggcgtccatgaatgtctctacaagactcatgacgtgcaaggtgag |
| | | ttcgtccgcggcgcgcccttctacagacggggtgagtgggccaaggaatgacttttcgtcgtgtcgatttatcgcctagttacgct |
| | | tttggtcttaagttgcgttcctaagagaggtgggctgtgtccgacaatgcgtattacgtttatgcgctgaaagatccacggatggc |
| | | gcccgcccagccgttctacataggtaaaggaaccgggacgcgctcccatgaccatcttgtaaggccagacgattcaaagaagggaa |
| | | gcaagatctccgagatcatggcctcagggcgtcaggtgctggtaacccggctcgtggacgggctcacagaagagcaagcgttgaga |
| | | attgaggccgagcttattgccgcttttggcaccctcgatactggggggatgctcctgaattccgttctgccaagcgggttggtaaa |
| | | caagagccgtagctcgctggttgtcccgtctggcgtaagggagaaggctcagattggtctggcccttctaaaggacgccgttctgg |
| | | agctggccaaggcgaatccgactggtatctcgaactccgatgctgcgagcatgctcggcctgcgtagcgactacggcggaggatcg |
| | | aaggactatctgtcgtacagcctcctcgggctgctcatgcgggagggaaagctcgctcgggttgccggcactaagcggcacgttgc |
| | | tcaagtgagctagctgtggggttccggatcgggctggcccgctcggcgctgcgctacgaagctcgcttgcctgccaaggatgctgc |
| | | ggtcatcgaacgcatgaagcactacgccgcgctgtatccgcggttttgctatcgccggatccatatctatctggagcgcgagggct |
| | | tccatctcggctgggaccggatgtt (SEQ ID NO: 60) |
| 64 | pLG066 | gatggactggtactgtagattcaccgtggaccagcgaatctattatgtggtgagcagaacattaacacatcaatgtaacgccgtaa |
| | | tcattgagtctttgccggggacgcttgacatctccgaaagaattatatcgtgagtcttaaggggaatctcttgcttccggttatac |
| | | atttaaccggatctagctataagactgttacatctattgggattaggtcaggacagatagcctgaaagcttttatagtgagggact |
| | | tcagaaataccctagaaaaggaactgttatggtaggttcgcgctggtataaatttgattttcataaccatactccggcttcgcatg |
| | | attacaaaattcctgacatcagccccagagagtggcttctggcttatatgaaacagcatgtcgattgtgttgtaatcagcgatcat |
| | | aacagcggagcctgggtcgacgtgttgaagggtgagctggagaatatgtcccgggacgccagcaccggcgacctgccggaatttcg |
| | | gccactgacactctttccgggggttgaactgacagcgaccggtaacgtacatattctggctgtgctgcacacgcacagtacaagtg |
| | | ccgatgtggaaaggcttctggcccagtgcaataataatagccccattccgagtgaagtccctaaccatcagctcgttcttcaactg |
| | | ggccccgccggcatcatcagtaatatccgccgtaatccgaaggctgtttgtattcttgcgcacattgatgcagccaaaggtgtctt |
| | | aagtctgactaatcaggcagagctcaccgcagcctttcaggaaagtccccatgccgttgagattcgacaccgggtggaggatatca |
| | | ccgacggaacccgccggcggctgattgataatttaccgtggctacggggctctgatgcgcaccatcctgaacaagccggcgtgcga |
| | | acctgctggctgaaaatgtcatcccctgattttgacggactcaggcatgcactgctcgatccggaaaactgtgtgctgtttgatca |
| | | gctccctccggaggaacctgcgtcatatttgcgcagcctgaaattcagaacccgccactgccatcctgtgggtcaggattcggcct |
| | | cggtggaattcagcccgttctataacgctgtaatcggctcaagaggcagcgggaagtccacgctcattgaaagcattcgtcttgca |
| | | atgcgcaaaacagaaggtctcactgcgacccaggggagtaagctggaccagttcattcggacggggatggaagcggattccttcat |
| | | cgaatgtattttccacaaagaaggcacagatttccggctcagttggcgaccagacagtaagcatgaattacatatcttcagtgacg |
| | | gagaatggatgcctgacagtcactggtcggctgaccgttttccactctcgatttacagccagaaaatgctctatgagctggcttcg |
| | | gatactggtgcattcctgcgcgtctgtgatgagagcccggtggttaacaaacgggcctggaaagagcgctgggatcagctggaaag |
| | | ggaatatctgaatgaacaaatcacgttgcggggcctgcgtgccagacagggaagtgcggattcgctgcggggggaattatcggatg |
| | | ctgaacgtgccgtcagtcagctgcagtcaagcgcctattatccggtttgcagacagctggccctcgccagaaacgagctgtccgca |
| | | gcaaccttacccctggagcactttgagcggcgtattgcagccattcaggctctggcagaagaaccgctgcagagatccgatatccc |
| | | gccggaaccttccggtctgctgatggcatttatggcgcgcctgtcatctgtgcaacagcagtatgaccagcggctcaatactctcc |
| | | tggcagaatatgctgcagagctcgcgggtatcaggagagagcaatcttttattgccctccgaacagcagtgagtgaccaggaaaca |
| | | aatgtagaaagtgaagctgtttccctgcgggccagagggcttaatcccgatgttctcaacgaactgatggcacgctgtgagtcact |
| | | gaaaaatgagctgagaaattacgacggtcttgatggggcgatctctgcctctgttgcacggtctgagcagttgctggctgaaatgc |
| | | gtgcccacagaatggcattgacagataaccggaaggcgtttctctcctccctgtcgctcagcgctctggaaatcaaaattcttccc |
| | | ctctgcgccccttatgaagatgttatatctggttaccagacggttaccggcatcagtaattttgccgaacgtatctacgataacga |
| | | tgacgggagcggattactgagcgactttatcagtgaacgtccgttcagcccgttgcctgccgcaacagagaaaaaatacagggcgc |
| | | tggacgagctgaaagcgctgcatcacagcatccggctggataattcagaggctggggcggggcttcatggttctttccggaatcgt |
| | | ctcaggagtctgaatgaccagcagctggatgccctgcaatgctggtatcctgatgacggcatccacatacgttaccagacccccgg |
| | | ggggcagatggaagacattgcctttgcttctccggggcaaaagggagcgagtatgctgcagttcctcttatcctatggcaccgatc |
| | | ctctactactggatcaaccggaggatgacctggactgcctgatgctgagcatgagcgtgatccctgccatcatgtcgaacaagaaa |
| | | cgccggcagctgattatcgtgtcgcactctgcccctatagtggttaacggcgatgcagaatatgttatcagtatgcagcacgatcg |
| | | cacaggcctgtatccaggactctgcggtgcactgcaggaagctccgatgaaggcactgatatgccgtcaaatggaggggggagaaa |
| | | aagcgtttcgttcgcgctatgagcgtattcttagctgaagaacggaaccgtccttaaggcggccatgaccggagagtgggcctggc |
| | | ggctgaatgcctggataaaagacgcaaatgtcagactgatggcctctgcgtctttg (SEQ ID NO: 61) |
| 65 | pLG067 | cctggtcctgccaattgctcccccagccatatgacataatccttttgaataatagggtttttatgcttgtactctagcccattcgc |
| | | ggtatcattttacgatctctcttccagttttatgcttaccgcctttgcctatcgtagaacaatgccgggaagcgttatcagcgatt |
| | | aagggcaaggaatgagaaaaagctggactatagaggaagattgtaagctgctaaccttggtgcgtcagctcttttccgcgctggtc |
| | | agccataaccggctgaatgccacaatgccatttagccagcagctccacgatgcatttgactcacctgaccgcgatgccgcagcatt |
| | | gctttatcgcctcgaacaggcaaaaatcttgggatttgccagccgtcctggtggcgatcccactaaacaactgtttcgctgcctga |
| | | taagcaatgatttggcgctatacgattacagcctcacctttcccaccctcagaaaagcattgcatccagataccgttgcggcagca |
| | | ctaaaccacttcacgattagcaatccacacgaaccactgtccaatactatcaatgaaatcgcgacagccttgcatcttgcccccat |
| | | acaggtggaaaagattctgatcgacagcggccaaataaccatcaatagttaccgcaagtgtgagcgtgttggagagaaaaatatca |
| | | ataataatctgcaagatctcatctctaggcaaattcctgacataacgctgattaaagagattaacgcctgtcgcgcccaagtctct |
| | | caactttaccacgtgcatgaacgtgatggcgctgaggtcatcttcagttccgacggcacggggttcggcaaaagctatggcgtgat |
| | | ccaagggtatgtcgaatatctggagcgcttcgccaaaacccaaaagtcagacgatctgtttcctgaaggtggctttaccaacctgc |
| | | tattcatgtcaccgcaaaaatcacaaatcgacctggacagcagtcagaaagagaaaattctggccgctagcggcgagttcatttgc |
| | | gttctctcccgtaaggatgttgccgacctcgactttatggactgggcctctggtctgaaaaaccgcgaccgctatattcagtggta |
| | | cgaaggggcgaaaggcagcaaatatatcggcggcgctatgcgttcgctcaattatcatgtcttacaaattgatcgctgtgaagagc |
| | | agttaaaaaagctgacaacatacggttctcaggataccaactacgaaagagaaattctcgaagaacagctaaaaaactgccgtcac |
| | | agtatccgcaatacgattgagtcagcctgtaaattactatttggaccagatagtgaaaaagcttccattaaagagtacattcgtcg |
| | | cgggctccaggcgcggcaagagcgaatgcaaaacgcggagacagcacgaaaaccaggaaagcttgaacctaagataagcgtacacg |
| | | aagtctatttcgagcttatcaaacaggtattgcctttcgaagtttgccagtaccgcccgtcagtgctattaatgaccacgaataag |
| | | ttcgacacatcaacttaccgactggcgcctcgtcagcgaggcgaaggtgtgcgttttgagtccgtaggtttcgacttgctgattgg |
| | | cggtaagctgactcccaaagatccacagattagcaccgttgcggcagccggtcataccgggcaggttacctatcttcgcgacgaac |
| | | acttcagacgcaatccagattgtccttttcgccagaaaaatattcgttttacggtgatcattgatgaactacatgaagcctacact |
| | | cgccttgaagaaacatgccatgtaaagctaatcacacaggaaaataacctggcgcacgttatttccgtcgcaggacgtattcacaa |
| | | cgcggtactcagcttagaacgccgaaacaagcccaaagaagcgcaaacgacctttgagcaagagatggtcaaattcatcactactc |
| | | tgcgcaatttactggcggaaaagtgcgaactatcccccggtacaaggctgggatcgatcctggagatgtttcgtgaccagttaggg |
| | | gcatttgaagtcaacggcgacgccgccgaacgcatcatctcaatcacccgcaacgtattcagctttaaccccaaaatgtacgtcaa |
| | | tgaagaagggctgaaacgcattcgcatgcgcaacagcgaaggcgacataacgcgcaccgaactgtattacgaagtcgaaaatgatg |
| | | ccaatgacaccaaccccactctgcacgatctgttccagttggtctccgtcatcctcgccgcctgttctgaaatcaccaaccggcac |
| | | tttaagcgctgggtaaagaatggtggccaggacaactccagcagccagaatacgcctttgggccagtttgttgacgcagccaataa |
| | | cgtagccggcgtggtgcgacatatcttcgatcgcaccaccgataaaaacttgttgattgatcatttctacacttacctgcaaccca |
| | | aaaccgtattcacgatgacgccgatagctgaactcaattacgtgaacaggggagccgagcgcacaattattctggcgttcgagatg |
| | | gatctggtacaagagttgcctgaagccatgctgctgcgtttattaaccggcacgcacaataaagtaattgggcttagcgccaccag |
| | | cggttttagccacaccaaaaacggtaacttcaatcgtcacttcctggcgcactatagccgcgaccttggctaccgggtcgttgaac |
| | | gcgaaaaggcagatatcgatacgcttaaggcattacgcgggttgagggccagtatccgcaacgtagacttcagggtgttcgatgat |
| | | aagcagttaaaattgaccgatatctaccaaaattgtgaaatctatcgcaggacgtatgacaactttttcgacgcgctgaagaaacc |
| | | gctggaatacgacctgaaaaatacctataaacggcgtcagtgccagcgggaactggaagcgttactgcttgccgcctgggagggta |
| | | aaaacagcctgattctgtcactttcagggacgtttaagcgggcctttatcagcgcctggcgcacgcaccagacaacctggcgtcag |
| | | cagtacggtatgcactcccggtgcgatgaaaaaacggataacggtaagaaacatgaccagatcctgacctttaccccattcaaagg |
| | | gcgtcacaccgtccatttggtctttttcgattcaccactggctaatgtcgaagatatcaggcaagaaacctatctccagaacagca |
| | | ataccgtactggtatttatgagcagttataaaagtgcgggtaccggcctcaactactttgttaaataccatgacggcgatattaat |
| | | gatatcaatgcaccacgtctggatgtcgattttgagcgcttagtgctcatcaactcctcgttttacagcgaagtaaaggacaacag |
| | | cggcaacctcaatacattacctaactacgttaccgtgcttaaacactacgccgatgacgatattaccgtccacaagctggccgatt |
| | | tcaacgttaatttcgcccacggcgaaaactatcgcctgttaatggccgaacatgatatgagcttattcaaagtcgtcgtgcaggcc |
| | | gtagggcgagtcgagcgtcgcgacactctattgaaaacagaaatctttttaccccgcgatgtgttccgtaatgttgcatttcagtt |
| | | cgccgctcttagtgaagatagcggtaacgaggtggtatcagaaagtatgtctttgcttaaccaccgactcatggaggagtgcgaaa |
| | | agctgagtcagggccagtcattcaataatgcggaacagcgactgacgtttgagcaagctatcgtcgcgaatggtcgccgcatcgat |
| | | gaaattcacaaacgtgtccttaaaaccgactggattaataaggtacgcgctggcaatctcgattatctcgagatatgtaatttatt |
| | | ccgcgatcctgactcctttaccgatccccagcgctggctggcaaaactccaggctaatcccttgtataccgccaatcgacaaatgc |
| | | aatctgttcacgacgctctgtttatcgatcgtcagcaagggaatcaaacgattttactttgccacaaacgcggcccggatggactt |
| | | gcccacagagattattccgccctgtcggatttcgctggcggcgcaagagagtaccggccagagctcaccctctttccgcagtatag |
| | | aaacgatgtcgattttacccccggcaacctggtcggcgagttgattcgtgaatgtgacaacatccaggaaaaggcattcaaaaaat |
| | | gggtacccaaccccaggctagttccgttgctcaaaggcaatgtcggtgaatatctcttcgataaagtgctaaaaagttatggtgtt |
| | | accccactctccgaccagcaggtgtttgaacgccttgaaccgctggtctatgagttttttgaccgctttattgaagtgggcgacga |
| | | cctgctctgcatcgacgttaagcgctgggcgacacagttggacgatttgacgcgggcagaagaaacgcttgagaaaagcgacaaca |
| | | agattcgccagatccgtaatatcgccagccaaaaggcggatactgaggggcagaaacagctccagacggcgctggcaggccgttat |
| | | gaacgtattcgatttatctatctgaacgtcgcctacagccagaaccctaataatctgatgtggcaggataatgtggatcacacgat |
| | | ccactacctcaacctgttgcaaactgactaccagtattatcagcccaaaaatcgagagagcggacgcgctcaggaaaactcgaaac |
| | | tgcgcatgacattggatataaacccaatgttactaaccctgctgggtgtagaaaagttgccgactaaaggaaaagtatcatgatcc |
| | | ctaatctgaatgagctgacggatactccgattgcccgtaccaatttgatcaagcttgaagaagatcagctgacaacaatccagcgt |
| | | ctattggccccggtatctaatatctatacgatagactttatggttcagcactttactaaagagcgaaaagaaaaatccgctgatta |
| | | ctatgcgcgaattcatcaggaggtaaaaacttgcgtgcggcagaagcttgggcttgaggccggacaggaagtaaaatatgagctgca |
| | | ttgcttacccaattaccatcacgtcttttttttcctggcgcctgctgctgcaccgaacagcctagcgcatcggactttggcagaacg |
| | | cattgaaacgctttgccagcgactcacagctgaaaattatgatttatctcgcctgattcagggattgttcagtctgcatttgaaaat |
| | | ggtaatgctggaacaagccagcgagcgcttttcggtaccgccaacctacttcaactctacgttctatctcaacgctcgcctgagtca |
| | | gcccgtcacgcagaaaagcggcactggagtgatggaggcattcgaactcgacatttatgcatcagaatataacgaactcgcctttac |
| | | cctgcacaaacgaaaatttctggtcgaaccggaggatgaattgcatctctctctggacgatacctgcgtgtggtttaacatcgataa |
| | | tcgtcggctcaaagcccggcgcaaactcgatgcccgggatagcaaactggacttttttcgtgagcgcagcggctatggtgaatgcca |
| | | ggcctatacctataacgtggtcatgaatgccgcctgcgagcggctcagtgaactagagatcccgcatcagcctatcgcatttcaggc |
| | | cacccacgaggtcaatcagttcgctaccgacctcgatcaacaactgactaatacgctgttggtggttaataacggcgtcgaatttag |
| | | cgccacgcaagaagcttatttctttgacacattagccatccagttccccgggtatcaactctggcctctggcgtcgcttaaacattc |
| | | tcagcaaaccggcttttctgagctgcctgccagtacatctattctggtactcaatgcagtagatgaagagcggagcaacagcatccg |
| | | ccagcaagataatgaatctgttgagtacaatgatttctatgcggcctttgccgacgcccgaaaacaacccgaactcaattgggatac |
| | | ttatacccagcttaaactagatcgtttgcaagggtggctaaatcagcaacctctgcccgtagtcttacagggtatgaatattgatca |
| | | caagttgttggatgcgattgattttattaatgaacaattgacaagcaaccctactcaatacgaaatcgatcttacgaagcctcacag |
| | | tcgtctcaagtcagcagttaccttacttaacagtaaggttcgccgaacaaaaaccgagctatggttcaaagagagcttactcaatca |
| | | gcatcacatcccactaccagatttggcggacgggcactataccgcctatgcagtacgcaaaacgaaaagctatctccccctgcttgg |
| | | atatgtcgaactaaaaatagaacacggccaacttagggtggttgataccgggatcgctgaaggtaaattagactatctgtctgttga |
| | | tcccccctctctgggacgattaaagaaattattcgacaaaagcttctatctctacgaccacacagcagatgtcctgcttaccaccta |
| | | caacagctcccgcgtaccgcgcctgattggcccggcgcaatttaatatcgtcgattcatacgcttatcaggaacaagaaaaaactct |
| | | ggcagagcgtaaaggggataaatttaacgggtacgccatcacccgctctgcaaaaccggatcaaaacgtactgccctatctgatatc |
| | | acctggccgctcgaaatacgactcgctgaccaaagcgcaaaagatgaagcatcaccatatttatctgcaaccgcatgagaatggtgt |
| | | atttgttctggtaagcgatgcccagcctacaaatcctactattgcacggcctaacctggtggaaaatctgctgatatgggatgccca |
| | | aggcaaagccgtagatgtatttagccacccgttaactggcgtttatctcaatagctttaccctggatatgctcaggagcggtgaaag |
| | | cagcaagtgttcgatttttgccaagcttgcccggttgatggtagagaactagcggaaaatttagggcggtgtttttagaattcgtta |
| | | tgtgtgaacctaactgatctcccccctgaaaacagtaccagtctaaactgaagtctccggtctttcttcctgctcacagagaggctt |
| | | attaccatgaaaaagacccgttataccgaagaacagattgcgtttgcgctgaaacaggccgaaaccggcacccgcgtcggggaagtc |
| | | tgcagaaagatgggtatttc (SEQ ID NO: 62) |
| 66 | pLG068 | caactgaggcggatatggccggtgcgttcatgtcctgaattaattcgaaagacaaatcgcgttaccaagcgttgcgcgatttagca |
| | | gcaaattgatagcttagccaccaacatttacacgttgtaggttgtcttggccgccattggtcttcagcaacctgcaacgctgatca |
| | | gtcgctcagggaagatgaggtaccgcagatggacaagcacgcccccgagcacctgctccgccttctagcccaaggcgcctcgctgt |
| | | gtggcaccgacagggccgaagcgtttaccgtgcttcaaagagcatccgcattgctctggcgcctggagcctagcgctccccccatg |
| | | tcagcgatcaagcttgaaaatcagcttagcctacccttggaaaagtggttgccggatgcactgaggctagattattcgggcccact |
| | | gctttactccaacatcgcgacgcagacctgcaacgaaatgctgcttgagctcgacgtcagtcagctctgggaagaagtccaggcaa |
| | | gcgtaaatagggtcaagcaggcgtgtcggctgcgggcagagggagaaattcactaccgcaacttcaggcttttcctcattgagcac |
| | | ggcgtgatctttccgtctgaagcccaagatgtcttcatcccgctcaacctctccctgaacgagttctacgaccccatccccctcca |
| | | tctgtatcacaacggcttagtctacttgtgcccggaatgccggtggccaatgaatgcccagcggcacgaagtcagctgcgactcag |
| | | cctggtgccaagacaaaaaaagcctttttgttcgtgaaggtacaagccttctcaaccgtgtgaacaacagcgtgctgcatggccag |
| | | ccggtcgatggccgtctgatgctcaaacctgcgctgtggaaattcaccctgcagccaggacttatcgaaatcgccctggcgagtac |
| | | gctggcgggaaaagggtttgatgtgagtctctggccggatgtggatcgaacagacctccgtatccagttaggccttattgagcagg |
| | | acatcgatgccaaggtttgggtgtccccttacgagttggccaaacacatcgaatcgatcccctccagcaaaccacgttggatcgtg |
| | | attcctgactatcagcgggagagcattccgtttctacgccagcgctgcaagtctggggtgagtgtatttacccaaagccagtgtgt |
| | | gaaggaggccctgaaacatgctccccctttctgataccagcgtcatactgttcctcgccttagccgcgcgttacgtcggcaacgaa |
| | | cccatggtggcggacgcagcagcgctctgcgcgggtcgcacacgaggctggagcacttggtacgtgctatccgagcctgaccagct |
| | | acttatcgctgaaggcttgcgcctacgcccatcctccgtggcgcagcccaaacgcttcgtgatgaccgcagaggaaattatcaagg |
| | | gcgaacgtagcccctttgagttagtcgactctggcaagctcagcagtgagctccacgagcaggattgctatcgcgtttcaccccac |
| | | ctgaacgtcgatcagctcatcagggagcacctagatgcgttgagatatgggcgccccccatcggttcatgcacagattccagactc |
| | | aggggatgtcgttctcaagcacatcacaggtgatcaggtcagggtgttcgtcgtcccacagagcgagcgaggggtgctcagtggcg |
| | | cccaccagtacgttactgtcccaacctcccatgcagcccctgagacgaagtgggaacttgacgctctgaacgagctcgcggagtca |
| | | ctcgatggtgcaaccggattgcacacgaatcatcgaagctcgttggccaacatttggggttcggatccgctacgcacagctgacgc |
| | | aggtcatttttatcgtgtgaacgcgccgactggcaccggtaaaagtgtggctatggtcatgatgtcgatcgatgctgctcgcagag |
| | | gacaccgggtggtgatcgcggtgccaacgttggttgagcttgagaacacggttcggattctcaagcaatccgctgcggtgacagcg |
| | | cctgatatcacggttgcccccctgcactcagcaacacgcgtatacgagcgcggaaagcttcaatttcagcagggtcattctgcacc |
| | | ggcctacgactatgcctgcttactcgatgcctatgcctcggatacgctgcaagttgaacctggaaaagaaccgtgctttaacgttc |
| | | gggtatcgacacaggaagaaggtcgtgcagaacaatcaaagcggctgaatcactgccctttcctgttcaagtgcggacgaacaacg |
| | | atgctgtcgcaagctctggaagcggacgtcgtggtgattaaccatcacgccctgttgtccggaacaacccgcattccattgtccga |
| | | ctcagaccggtgtccaggcccacgcagcttcatagagctgctgctaagaacagcaccggtgtttcttgtcgacgaaatcgacggtc |
| | | tactgaagtctgcgatcgacagcagcgtcatcgaattgaagctgggcaatcaaggtgacaacagcccgctgctccgtctattcaat |
| | | acagtggccggtcgatccagcattcctgagattgatcgaagcagcatgtaccgcgtgaactgggcgcttacctactgcacgctgag |
| | | tgtcagccagctaatgaacctccagcaagaggaatatttcgagtggccaaagaaagaaaccacttggtcggacgcagacgacacgt |
| | | tcattaccgaaaagcttggtattgatcgtgagacgcttgagcacttgttcaacagcacgaaccgcataccgggctatctggaaaag |
| | | ctgagtcaccaccttgctcactggcaatcaaatgggggccagtacaagcttgaggccttggcaatcaatctgggccatctcgtcaa |
| | | agagttgtccgacagcgacttgcttcctgcgcgtctcaaggagcacgatcaaatccgcctcaaggcgtcactcatcttgcgaggca |
| | | cgttagaagcgatcgaaacgcacctgcgcaaccttcaggtcgagctacccagcttcgtgaacgccgaaataccttatgcctacgag |
| | | gtcaaacggagtatcgcagggccggagccgctgagcccgactccgaatggccccttgcagcgagccgtatttggcttcaaacgtaa |
| | | agacaccggagacaacgactcaactctgaacgttgtcgcaatgcgtggggatccgcacagcacactgctttcgctgccagatgtca |
| | | gcgccttgggctatgccggtgtaaagcgattgtttatcggcttctcggcgactgcctacttccccggcgctagcgcttacgatctt |
| | | cgtgctaaggatttcatcgacgttcccgatgtagctggccaggtgactttcgaaaatgtgcctcagacaaccgctatctctggcgc |
| | | tcagttctcgcagcgaaaattcctggtatcaaaattcgccaaagagatttggccgtggctacgcagccgacttgcaagcttggcca |
| | | acgaccccgtcacgcagacgcgtgcccgcctgctgctggtcaccaatagcgatgcagacgctgaagttctggccatgaccctggcc |
| | | aggatgcagggcggtcctggtcagctggtaggctgggttcgtggacggcaaagcgactacaagccgtcctcgctagatgcacagca |
| | | gatgcttgcatacgatgatctcgctgagttcaccaacggccgacacaaggacaaaactctgctggtcagcgccttgggcccaatgg |
| | | cgcgtggacacaacattgtgaacagcgacggattttcagccattggtgctgtggtgatctgtgtacgccctcttccatcgtcagat |
| | | agccccaacaacaatctggcgcacatctgttacgaaaccagcaagtttgtagcgccatccagcagtccgggcgtattgatgatgca |
| | | ggaacggaagcattccaatgcgctgctgcaaaagattcgtaccgcccgccccgcgttcagccagcagccggccaacatccgccact |
| | | acacgatcatgaacatccttgtgagcctcacccaactgatcggtcgtggacgccggggcggcacacctgtgacttgctacttcgcc |
| | | gatgcggcatttctcgaaggtttgaagccgtggcctctgatgcttaacgagagcgttgaacagctcaagcaagacggcgattggaa |
| | | ccagtttgcccgtcatcatgccggcgttgcatcggcacttttgaaatacatcaatggatcagtgaaggacgcacgatgaaggttct |
| | | tgaattacgcaccagcctctttgagttcgatccagcagctttgggacaaagctaccgcgtcgtggtaggcccgcattaccttgatg |
| | | cctggcaagctcttcagggactggtaaggaaaccccatcctggcctaccgaccatagggcttgaggaaatgctcgccaccctctct |
| | | ggagggccggtcaaggtgaacctgtttccgcaaaaagaaggaggcgtctcggcgatccttttgctgaagcccctgcccgttgacac |
| | | catcaacgaagcgctccgcctttgggctatggacgtgatgcagttttacaaacaagaactgctcgaattcgaaggcaaactggtcg |
| | | tcaccgacctggtacctatggacactgcccgcttggtcgcgtccggtgacgtatcgtcccttgcgtacacagtcattccttggttg |
| | | gtaggtcaagcgctgattgcgaagccaatgcaagcagcgaaacctcttaagctttatcaggctgccgacgggtgcgtgctcgcctg |
| | | ggacgacccagtcgtttcggaaagcgacgtacgctacgccagtgcgcttcacgccatcgagcctgcattggtgctgatctacggcc |
| | | aatccaagccctatctacagctgcgggtaaagctgactcaggtgatgccgaatctcaagggtcaaaagaagcatgcctgggtcaaa |
| | | actggcgacctgattgtcaaagcaaaaatccggagcaagcccgacgggcatgggggctgggaaacattttacgaacatcccattga |
| | | aaagttgctgacctttatgggggttccgtcgtttcctccaataatcgagggcgatatccctgtcgacagcgacgtgcgccctatct |
| | | acgccattccaccctcgaaccccttgatcgcgtcaggcactggccccctgtttcttgaccaggcaggattccatctgcttgcttgt |
| | | ctaccaaggacaaagccgcttctggtcagaaaatctgtcgctgttctgcgcgaagaaaagaccaatgctacgggcgaggtgatcga |
| | | cttgaacgtgatggtcttggcagctcacgcagacgtgatgctaaggcttcacggggcgagttcaaacttggccagggacagcaagt |
| | | tcttcaagaaagtcgccccaccacgtgtgacgctgtcacgtctggatgtgccagatgcgcagcgtatgctggaggggcagcatgac |
| | | ctgaacagcctcaacgaatggttattgaatcacgtggttccggcgagcagagtgctcgctcaaaacggcgccaaggtcatgattgt |
| | | tgagaccagtgcatcagcagcatcacgcgaaactggactcgatcccaagcacgtcatccgccgggtgctggcgaagcatggcatcg |
| | | ctacccaattcattatgcacgttgaccccgatgcacaggtgaagaagcgcaagcctaaggaagatgaccgtgatttcaaagcgatc |
| | | aactcgatcatcgaagcgattcggttgagcggccagcaccctgcccctacacccaaggtcaagtcgatgccggccaacactacggt |
| | | agtttcagtcctgctagatcgactccaggacaaaggctgggcgaaatttctacccgtgatcacgcgcaccacgctcggtggccaca |
| | | cccctgaaatcttctggtttgagtctggcgcagagtctgcaggcaaatggttcagctacagcgcgggactgactgcgatccatgcc |
| | | acggacacgctgctgacgcctgatcaattgaaaacactgatcacccaagcccttcttgattgcaaaatcaatccagctgactcgtt |
| | | gatcgtctgcctcgatgcagacctgagaactttttatgcaggcctaaaagacagtcctggtgaggggctaccaaccgtaccggacg |
| | | atgcagcagtagtgcgaatccgtgcggaccatcaggtagcacagatcagtggtagccacaccttgtcgccgcaagcagcccactac |
| | | attggcacgaaggtcggcgcgttccagtcctgtgagagtccctcagtgttttactttgtgtctccatccaagcagtttggcagcgt |
| | | tcgttcgcagcgtgacaacacccgttacgacgtacgggagagagatcttcgggatccttggcaacagctcggcgtcacggaaattg |
| | | ccatcatccagcctggagcctttgacggtgcagctgcggttgccgagcaagtggcgttgctctgtcgcaacccaccactgtgggat |
| | | ggtcatctgcgcctgcctggcccgatgcaccttggcaagcaagtagctgcagatcatccagttatggaagcgcggcgaaagacaga |
| | | ggctaatcgatcagccggttaaagccgcctggtaaccgttcattactagacacgtataagtcataacacccagcatttcacaaaga |
| | | gcgcga (SEQ ID NO: 63) |
| 67 | pLG069 | atttgcctgagacttatttcccgtggcgcttagctagctaagagtgggcatcgtgagcaccattgatgatatgaaatgacggtata |
| | | gcaatttaaccgtctggatttcaccagaaattagtgattcaataggaaattaaatacgttttatatttcaatgtgtatcaaaatca |
| | | ttcctgaaatttcctggtgctatatttgatgaaaacggataaacattctgttgattttaataaaattctgtctttcgatttagagc |
| | | ttacgcgtgatgaaaagttaaggcatatgggggccgtgctggcggaacgcacgttgagtttgaagataaatcaggatgaagcgatt |
| | | catcaattggatgaaatggcaggcgatgcagatttaatcctcggtcataacatactggatcatgatttaccctggattgccaaaca |
| | | acgcgtacgtgctcaaatattattagataaaccaatcattgataccctttatttatcaccgctagcttttcccgcaaatccatacc |
| | | atcggctgattaaagactataaactggtaagagatagcattaacgatccagtgaatgacgctaaattatcgcttcaggtattcacc |
| | | gagcaaatatgtgcgctgcaagaaaagccgctggctcagttgcagctatatcagtatctttttgagcacggcgttgccagccattt |
| | | cagtacacgtgggatggccagcattttttccgcactgacgggtcaggcgtccatatccgccgtagttttacctacgctagttaaat |
| | | cggttgctcagaataaagcatgccctaaccagcttaatcgggttattggcgatgctcttaaacagcctttgcgcttactaccattg |
| | | gcttttgcctgtgcctggctccccgtatcgggagggaattctgttttaccgccctggatatggcgccgttttcccgtcaccgctga |
| | | tatcatccgcgaactgcgtgagcaaaaatgccagtctgaaacttgccgctactgctgtgaaaaccatgatgctcgtcggcatttac |
| | | agaaaattttcgagctgaacgattttcgtaaacttcctgatggctcgccgttacagcgcaatatcgttgagtacggattagctagt |
| | | cgttcactgcttgggatattaccgactagcggagggaagtctttatgttatcaacttcctgcgattgtcaggaatctgcgaaatgg |
| | | ttctttaaccattgttatttcgcctttacaagcgctgatgaaagatcaagtggataatttacgtcataaggcaggtattaaaggcg |
| | | ttgaggccatttcagggatgctaactttacctgagcgcggcgctattcttgagcaggtccgtaagggggatattgcgattctttac |
| | | ctctctcctgagcaattacgtaaccgcgcggtaaaacaagctatcaagcaacgtcagattagtggatgggtttttgatgaggctca |
| | | ctgtttatcaaagtggggccatgattttcgtcctgactatctgtattgtggcaaggttattgaatctttggcgcaggagcagtctg |
| | | tgcagattcctccggtattttgctataccgcaacggcgaagttggatgtgattaatgatatttgtcggtattttgacaaaaaatta |
| | | tcgcacccattagctcgtttttcagggggagtagaaagaattaatcttcactatgaaatcattgcaagtaatggcttgagcaaaat |
| | | tagtcagattttgaatttgctcgataaatttttttctaatgatgatgaaggtgcatgcattatctattgcgcgacccgccgttcgg |
| | | tagatgaaatcagcgatgtgttgacccaacagcaacctttaccggttgctcgtttttatgcccggcttgaaaatagtgaaaagaaa |
| | | gaaatccttgaagggtttattgctaaccgttatcgagttatttgtgctactaatgcctttggcatgggaatagacaaagaaaatgt |
| | | acgtttagtaatacatgcggagatccccggttctctggaaaattatctccaggaggcagggcgtgctgggcgggatacgctggacg |
| | | cgcattgtgtgctattatttgatgagcaggacattgaaaaacagtttcgccttcaggctattagtgaagtaagctttaaagatatt |
| | | tatgcaatatttaagggaatcaaaaagaaagttaatgaaaataatgaagtcgttgccacaagtattgagctaattaatcatcctat |
| | | ggttaaaaccagtttctctatcgatgataacaatgcggatactaaagttaaaacggggatagcgtggctggaacgtgttggttatg |
| | | tggagcgacttgataatataactcaggtttttcagggaaaagtggcctttccttctctggaagaagcgcaaagtaagatggcagcg |
| | | ctgcacttgaatcctgcggcgatggttctctggaatgctgttttacaggcgctattaaatgctaatgacgatgacggacttagtgc |
| | | cgacagcattgctgatgaggttgcccaatttcttccgcataaagaaaataatacgtcaggaattgaagcaaaagatgttatgcgcg |
| | | tattgacacagatggctgatgttggcctggtcaccaggggaatgctgctgaccgtacgtatgcgccccaaagggaaagataatgcg |
| | | aggatcacaactgagttaattcacaatattgaaatcgccatgttagggctgctgcgcgaagctcatcctgatattgaactggggat |
| | | gccatggcctctccagattgcggttatgaatcaagagattattcagcaaggctatgatagaagtaataccacgttactacaaaata |
| | | tattatttagctggtctcaggatgctcgagcaaacggtcataaagggcttattgattttcgttatggtacaaggaacagctaccag |
| | | attattatgtatcgtgactgggcatatatcgaaagagccattttacaacgtcatcgtgtgacaagctccgtactgaattttattta |
| | | tcaattggcattggatagtgatgaaagcagtatcaaaaaagtgatgctttctttctcactggaacaggttatcgattatttaagaa |
| | | aagatgttgatattattccaatgatccaacagagacaggggggggatgagcagcagtggctgatggctggtgcagaacgtgctcta |
| | | ctttatcttcatgaacaacatgccattgtgctgcaaaatgggctggctgttttccggacagcgatgagcttgaaattgcaggctga |
| | | aaaatcgcaacggtatgtcaaagctgattatgaaccactggctctccattatcagcaaaagacgcttcagatccatgtgatgaatg |
| | | aatacgccaggcttggtcttgaaaaacctaactatgcccaacggctcgtacaggattactttgctatggatgccgagtcatttgtt |
| | | ccactttattttaaagggcggcgaaaaattctcgatctggcaaccagcgaaagctcatggaaacgcattgttgaaaatttgcataa |
| | | tcccgatcaggagcaaattgtgcaggcgagccttgaacaaaatacgttagttcttgccggaccaggctcagggaaaagtaaagtta |
| | | ttatccatcgatgcgcctatcttttacgcgtgaagcaggtcgacccgcgtaaaatcctgttgctctgctataaccgtaacgcagcg |
| | | atttccttaagacgcagattgaagtcgttgcttggtaaagatggcgccagcataatggtacaaaccttccacggattagcattgag |
| | | ccttacgggataccagattgagcggaaagataatgacgaaatcgattttgataacctgctctggaaagcaatagctttactcaaag |
| | | gcgatgaaacgcagctcgggttagaagttgaagaacaacgtgaatacctcctcggcgggcttgagtatttactagtggatgaatat |
| | | caggatattgatgagccacagtatcagctgattgccgcgctggcaggtaaaaatgaaagtgaagatgatgctcgtcttaatctcat |
| | | ggcggtgggtgatgacgatcaatctatttatggtttccgtgatgccagcgtgcgatttattcgtttgtttgaaagcgattactccg |
| | | cccgtactcattttttaacgtggaattaccgctctacggccaatattattgcatgttcaaattatcttatcagtcataatcagggg |
| | | agaatgaaatgcgagcatccgatcgtaatcgatcgcgctcgccagatgcttccgccaggcggagagtggagcgcacttgaaccttc |
| | | ggaaggcaaagttgttatccagcattgtaccggcgcggctcagcaggcggcagaagtcgtgcgccaaattcagtatattcaacggc |
| | | tgcagccggaatgccctcttgagaaaattgcggttattgcacgcaatgggctcgacaaaaaggagcttatttgggtccgttcagcc |
| | | cttgcggatgcaggtattccttgccgctttgcgctggagaaagattatggtttccccattcgccactgtcgggagatcgccaatta |
| | | tctgctatggctacgagaaagagcgctcgagtcgctgacgccagcagagctgtgtcagcaactaccggggcgagaccaggcgaacc |
| | | gttggcacgatattatttatgaattaattgagcaatgggagctaagccagggaggcgagccattacctgccgcttattttgaacat |
| | | ttcatactggaatatttacatgcccagcacagccaggttcgctttggcctgggggttttgctgagcaccgtacatggcgtaaaagg |
| | | tgaagagtttgagcatgtcattatattagatggaggttggcgtagttcgcactctctgcaacctgaaaataacgaagaagaacgaa |
| | | ggctcttttatgttggcatgacgcgagcgatatcccgacttgttattatgcatgatgatcgtgcgccaaatccctatatcgaacag |
| | | ttagatccagcggtcatcagccatactgctgcacaagccgttgcgcctgggatcttacgtcgtttctcgatcatcggattgcgcca |
| | | gctctatatcagttttgcaggtggacatccggctggtcatcccattcattcgttacttaccgatatgcaggttggggatagcgtcca |
| | | actggtctctgtcgggaataccatcaaggtgaatgctaatcaatcggcaattgcgcagctttcaagtgccggaaagagccagtggca |
| | | attttctctttccgggatccgcaaaattgaagtgcttgccatgctacagcgcagcaaaacactaacagcagaggattatcaagttgc |
| | | ggtgaaagtggacaattggtatgtaccgatattattggttgaaacccgtgaagaagccgcttatgacaatattacttgaagcagaat |
| | | ac (SEQ ID NO: 64) |
| 68 | pLG070 | tagctattgtgactatgctaaccatatgaatctattgtgtgattatgagtaatgactttttctaatatttgatttttaatgtagta |
| | | acttagctaattttaaaatttgtaaaaggatgtttatgtcgatttatcaaggtggtaacaagttaaatgaggatgattttcgttct |
| | | cacgtttattccttgtgtcaattagataatgttggcgttctgttaggtgctggtgcttctgtcggttgtggtgggaaaacgatgaa |
| | | agatgtatggaaatcgtttaagcaaaactaccctgagcttttgggagcacttattgataaatatcttctggtttcgcaaattgatt |
| | | ctgataacaatttggtcaatgttgaacttttgatagatgaagcaactaaatttctttctgtagctaaaactagacgatgtgaagat |
| | | gaagaggaggaattcaggaaaatattaagttcattatataaagaggttacgaaggctgcattattaacaggagaacagtttagaga |
| | | gaaaaatcagggtaaaaaagatgcgtttaaatatcacaaagagttaatttcaaaattaatttcaaatagacagcccggtcagtcgg |
| | | ctccggcaatttttacaacaaattatgatttggccttagagtgggctgcagaagatttaggaatacagttgtttaatggtttttct |
| | | gggctacatacacggcagttttatccccagaattttgatttggctttcagaaatgtaaatgcgaagggcgaagcaagattcggaca |
| | | ttatcatgcgtatctctataaattacatggctcacttacgtggtatcaaaatgatagcttgactgttaacgaagttagtgcatctc |
| | | aagcatatgatgaatatattaatgacataatcaataaagatgacttttatcgcggtcaacatttgatttatccaggggcgaataaa |
| | | tatagccatacaatcggcttcgtttatggagagatgtttagacgttttggggagtttatttcgaaacctcaaacagcgttgttcat |
| | | aaatgggtttggtttcggtgattatcatataaatagaataatattaggcgcgttactgaatccatctttccatgttgttatatatt |
| | | atcctgaattgaaagaagcaattaccaaagtaagtaagggtggtggttcggaagctgagaaagctattgttactttaaaaaatatg |
| | | gctttcaatcaagtaactgtagttgggggaggaagcaaggcatattttaatagtttcgtagaacatctaccataccctgtgctctt |
| | | tccacgagataatattgttgatgagttggttgaagcaattgctaatctttctaaaggagaaggtaatgtccctttttaaacttact |
| | | gaaatctcggctattggatacgttgtaggattagaaggggaaagaattaggataaacctgcatgaggggttgcaaggcagattagc |
| | | atcgcatagaaagggggtgagctcagtaacgcaaccaggagatcttattgggttcgatgcaggtaatatattagttgtcgcaagag |
| | | tgacagatatggcatttgttgaagcggataaagcgcataaggcaaatgtaggcacatctgatttagctgatatacctctaagacaa |
| | | attatcgcctatgcaattggctttgtgaaaagggagttaaatggttatgtttttatatcagaagattggcgcttacctgcattggg |
| | | ttcttctgctgttcctttgacttcagattttttgaacatcatttatagtattgataaagaagaactcccaaaagcggttgaattag |
| | | gtgtggattctagaactaaaaccgttaagatatttgcaagtgttgataaattattgtcgcgacacttagccgttcttggtagtaca |
| | | ggatatggtaaatcaaatttcaatgctttgttaacgaggaaggtttctgaaaaataccctaactcaagaatagttatttttgacat |
| | | aaatggtgaatacgcgcaagcttttacaggtattccaaatgtaaagcacactattctaggggaatccccaaatgttgatagtttgg |
| | | aaaaaaagcagcaaaagggtgagctatatagtgaagagtattattgttataaaaagataccatatcaggcattaggttttgctggg |
| | | ttaattaaattattaagaccaagtgataaaacacaattgcccgcattaagaaatgcattaagtgcaattaatcggactcattttaa |
| | | aagccgtaatatttacttggaaaaagatgatggtgaaacttttcttttgtatgatgattgtcgtgacacaaatcaaagtaaattgg |
| | | ctgagtggttggatttattaaggcgtagacgtcttaaaagaacgaatgtatggccaccgtttaaaagtttagcgactttggttgct |
| | | gaatttggatgtgtagctgctgaccgttctaatggaagtaaacgtgacgcgtttggttttagtaacgtgttgccattggtaaaaat |
| | | catacaacaacttgcagaggatataagatttaaatctattgttaatttaaatggagggggtgagctagcagatggtggaacgcatt |
| | | gggataaagctatgagtgatgaagttgattacttctttggtaaggaaaaaggacaagaaaatgattggaatgttcatatagttaat |
| | | atgaaaaatttggcacaagatcatgctccaatgttacttagtgcattgttggagatgtttgctgagatactatttagacgtgggca |
| | | ggaacgttcgtatcctacggtacttttgttggaagaagcgcatcattacctgcgtgacccttatgctgaaattgactcacagatta |
| | | aagcatatgaacgacttgctaaagaaggtaggaaattcaaatgctctttaattgtcagtactcagcgaccctcagagctttctcct |
| | | actgttttggcaatgtgttcaaactggttttcgttacgtttgactaatgaaagagatttacaggctctcagatatgcaatggaaag |
| | | cggtaatgaacaaatcttaaaacaaatatcaggtttaccaagaggtgatgctgttgcatttggttctgcatttaatttgcctgtaa |
| | | gaatttcaattaatcaagcaaggccagggccaaaatcttcagatgctgttttttctgaagaatgggctaattgtacagaattacgt |
| | | tgttaattacctgatgtacatggctagtgcaagttggtagcgcatgtctatatgcatttatttgcatgtgttttattgagtgagcg |
| | | cacaagcttgatgacccgacaggtatgtatttagactgaa (SEQ ID NO: 65) |
| 69 | pLG071 | gtgcgccttatgtgattacaacgaaaataaaaaccatcacaccccatttaatatcagggaaccggacataaccccatgagtgcaat |
| | | agaaaatttcgacgcccatacgcccatgatgcagcagtattgaaaaatataacatatccaactgattgtattgaaaatttaaaata |
| | | gccatataacaaaaggttacacataagctactttttggggtttcaggcaagaaactaaaaattattaacgccatcaaattattcac |
| | | atcttaataattagcattgaaatttaatgtttttggttctttgtacatgtcaatggcttgtctttgtggcagaatcataaagctat |
| | | gcaatcattgcattgttattaacacagcatatttttatatacttttaacaccttacctcaaaaaggataacaaagtggacagaagt |
| | | gcggttgatacaattcgtgggtattgttatcaggttgataaaacgattattgagattttttcgttaccacaaatggatgactcgat |
| | | tgatatagagtgcattgaagatgttgatgtctacaacgatgggcatttaactgcgatacaatgcaaatattatgaaagtaccgatt |
| | | ataaccactccgttatatcaaagcccataagattaatgttgtcacactttaaggacaataaagaaaaaggggctaattattatctt |
| | | tatgggcattataaatccggtcaagaaaagttaacactcccattaaaagttgactttttcaaatctaatttcctcacctacaccga |
| | | aaaaaaaatcaaacatgaataccatattgaaaatgggcttaccgaagaggatctacaagcctttttggatcggttagttataaata |
| | | tcaatgcaaaatcatttgatgatcaaaaaaaagaaactatacaaataataaaaaaccatttccaatgtgaagattatgaggcagag |
| | | cattatctttattctaatgctttcagaaaaacatatgatatctcttgtaataaaaaagatagaaggataaaaaaatctgattttgt |
| | | tgaaagtatcaacaaatcaaaagtcttatttaacatatggttttatcaatatgaaggaagaaaagaatatttaagaaaattaaaag |
| | | aatctttcatacgcagaagtgtaaacacctcaccttatgctcgttttttcatcttagaatttcaagacaaaactgatataaaaaca |
| | | gttaaagactgtatatataaaatacaatcaaattggtctaatttatctaaaagaacagatcgaccatattctccttttttactttt |
| | | tcatggcaccagcgatgccaatttatacgaattaaagaatcaattattcaatgaagatctaattttcactgatgggtaccctttta |
| | | aaggaagtgtatttacccccaagatgttaatcgaaggtttttcaaataaagaaatccacttccaatttatcaacgacatagatgat |
| | | ttcaatgaaacactgaacagtattaatataagaaaagaagtttaccagttttatacggaaaactgccttgatatcccatcccaact |
| | | accccaggtaaacatacaagttaaagactttgccgacataaaggagatagtgtaatgagcaggaataatgatattaatgcagaagt |
| | | agtatcggtatcgccaaataaattaaaaatttccgtagacgatcttgaagaatttaagatagcagaagaaaaattaggtgtaggat |
| | | cttatttaagggtttcagataatcaagatgttgctcttctggcgatcatagataatttttctattgaagttaaagaaagccaaaag |
| | | cagaaatacatgatagaagcaagtccaataggtcttgttaaaaatggaaaattctatcgcggtggagattcacttgcacttcctcc |
| | | taaaaaagtggaaccagcgaaattagacgaaataatatccatatactcagatagtatagatataaatgaccgttttactttttcaa |
| | | gcttatcgcttaataccaaagtatccgtacctgtgaatgggaatagatttttcaataaacatatcgctatcgtaggttcaacgggt |
| | | tcaggtaaatcccacactgttgcaaaaatacttcaaaaagccgtagatgaaaagcaagaaggttataagggattaaacaattctca |
| | | tataattatttttgatatacattctgaatatgaaaatgcattccctaattcaaatgtattaaatgtagatacattaacccttccat |
| | | attggctattaaatggtgacgagttagaagagctttttcttgacacggaagcaaatgatcacaatcaaagaaatgtgttccgtcag |
| | | gcaataacattaaataaaaagatacattttcaaggagatccagccacaaaggaaataataagctttcactcgccatattatttcga |
| | | cattaatgaagtcatcaattatattaacaatagaaataatgaaagaaaaaataaagataatgaacatatttggtcagatgaggaag |
| | | gaaatttcaagtttgacaatgaaaatgctcataggttattcaaagagaatgtaactcctgatggaagttcagccggtgctttaaat |
| | | ggaaaacttctcaattttgttgatcgattacaaagtaaaatatttgataagagattagattttattctgggtgaaggtagcaaatc |
| | | cgtaacatttaaagaaacattagaaactttaataagctatggaaaagataaatcaaacataacaatacttgatgtaagcggtgttc |
| | | cttttgaagtacttagcatatgtgtatcattgatatctcgattaatttttgaatttggctatcattcaaaaaaaataaaaagaaaa |
| | | tctaatgaaaaccaagatatcccaatattaattgtttacgaagaagcacataaatatgctcccaaaagtgatctgagcaaatacag |
| | | gacatccaaagaagcaattgagaggattgcaaaagagggtagaaaatacggagtaacccttctccttgcaagtcagagaccttctg |
| | | aaatttcagaaacaatattttctcagtgtaatacttttatctcaatgcgattaactaacccagacgatcaaaattatgttaagcga |
| | | ttactcccggatacagtaggtgatattacaaacctcctaccatcgctcaaagaaggtgaggccttaatcatgggggattcaatatc |
| | | aataccttcgattgtaaaaatagaaaaatgtacaatacccccatcgtcaattgacatcaaatatcttgatgaatggagaaaagaat |
| | | gggtagattcggagtttgataagataattgaacaatggagtaaaagttaatttcagaagtggattcactcttgctcaagagtgaat |
| | | ccactaatatcatatcctaatgatatagtttaataaaatctattctggaatcattaggctgagag (SEQ ID NO: 66) |
| 70 | pLG072 | ccattttttaaaataccctcttaaaggagggtattttaaaattatttgttttaataaaaattaaatattatattcattatcacaac |
| | | caataaaccgtttattttttacacttgcatactataaagacatgaaagatcccccttgtcaggactacgctaaagataataataac |
| | | gtctattttcgtcatatataatatttgcttgttgcatttctaaaaaaaaagagtaaaatatcaaaatttaggagttacttttggac |
| | | ttatatgaaggcaattgacttatttgcgggggctggagggtttagtttatccgcccacaatacaggcgctatagatgttgttgctg |
| | | ctatagaattcgatagcgcggctgcaaacacctacagaaaaaatatgttagaaaggcttgagcataagaccgaacttttacaggaa |
| | | gatattttactcgtaggcccaaaaaagttaagaaaaaaaataaagctcaagaaaggcgagcttgatatgatacttggtggacctcc |
| | | gtgccaaggtttttccagtcatcgaattaatgatgctggtgttgatgatcctagaaataaattacttttaaggtatttcgattttg |
| | | tttgtgaatttaaaccaaaagcttttttggtagaaaatgtctccggtttgttatggaagagacatgaagcccatttgaaacgcttt |
| | | aagtttttggcttccaaaaatggttatactttaattcattgcgatgtattaaatgctcgtgattatggtgttccgcaaaatcgcaa |
| | | acgagttttcattgcaggtgtcagaaatgacattttaaaaaaaagaaataatattgagtttccacctcaagctactcatttcaacc |
| | | ctaattctaatgaagtaaaaaacaattcaaaaaatacgtggagaaccgcatcctctgtttttgagaagatgaatgataacttaatt |
| | | caaagatatatatctgaatactttcttaaacatacttcttactcaattgatgaagcacaagagctacttgaaaacctagaatatca |
| | | agacgcacccataagcgaaaaagatccatgcaacatacatatgataccaactgagcgtatggaagagcgtttcagagccacaaaac |
| | | tcaatggcagtagaagcgatgcaggaaaagaatttgagctaaaatgtcattccaatggatacgcaggccataaagatgtttatggc |
| | | cgcataatgattcacctcccagccaatacaattacaactgggtgtaacaatccatctaagggaagattcattcatccatgggaaaa |
| | | tcacggcatcactttaaggcatgcggcaaggttgcaaacgttccctgatgactatattttttggggtaatgcgacagagcaagcaa |
| | | gacagattggtaatgcagttccccctatgttaggcacaatattaataaatgcattacttaacataattgcacccaatagataaggt |
| | | gtaatgtatgaaaaatatcaaaattagaaacttaaatggaccaaaaaatcatttgatgattacttaccttataataatagaaggtg |
| | | aaaaatggtaatttcagcagcttttcaaacaagagcaaggacaattgatcatctagggcgtgagcaaatagctgattgtccaaccg |
| | | caatttccgagctttggaaaaatgcatatgatgcttatgctcgtaatgtttctctaaatatatttgacggcaatacacctgtggca |
| | | actttagttgatgatgggcatggcatgtcgttagatgacattatcaataagtggcttacagtaggaaccgaatccaaggctacaaa |
| | | aaaagatattccatatgaagatagaaacggaatagatcatattcgagcaaagcaaggtcagaaaggcatcggtcgtctttcttgtg |
| | | cggccttgggctcattaatgcttttagtttccaaaaagaaagatagccctcttgtagcttgcctgctcgattggcgtatatttgaa |
| | | aacccatatttgatgcttaatgatataaagatacccattatggaatgcagtgataacaatgaattaatcactgttataccggaaat |
| | | gtttgatgctttgatgggaaatctatggggtgatggtgatgatatattacgagataaccgtattgaacaagcttgggaaaattatt |
| | | ctgaattagaaagaaatgaaaataattatattacaaaagaagctatcgagaatactgtaattaatgctttttttgaggaaaggcat |
| | | tttcaatcttggcctgtgtggaataataaaaccactcacggcacagccatgtttatagctggaattcatgacgatttaatagctca |
| | | gctatcaacagatgctggttcagaagctcaaggtgcagaggttcgggctaaagaacgctttcttcaaacattaaatagctttgtta |
| | | atccatttaaaagagaaggcgaagaacagattactgatttcaatacaagtgttgtcgcatggaatggtaatctgcaacgatttatc |
| | | atcgatgaagttagaaactttgatatttcaaactttgaccagctagaacatatagttgaaggaagtattgatgaaagtggattatt |
| | | ttccgggaaagtgaaagccttcggagaatggtttgataatattacagtcaaacctaaatctgcatataagaccagaaaagatactc |
| | | gctttggccctttctttttaagattaggcacatttgaagttataagaaaaaatagtacattatcagatgaacagcatgcaaccttc |
| | | gaccgtatccgtgatcagtttggtggagtaatggtttttcgtgatgatttacgtgttatgccatacggacgtgaagataatgactt |
| | | ttttgaaatcgaaaaaagacgttcaaaaaatgctggtttatatatgttcagtaatagggcatgttttggtggtgtatgtataacga |
| | | aagaacataaccccaacctacgagataaagcaggtagagaaggtataattgacaataaagcatctaagttatttagagagatagtc |
| | | gaaaacattttaatagaaattgcaaaaaggtttattggccgcgcatcaaatatacgagatgaaaagctagaggaaataaatgctaa |
| | | acatgctgctttgaaagcagacgaagatagaaaaaaattattacgtaaagagcaaagaagaatcaaaacatcgattcaaagagatc |
| | | gtatttctttagaacatttaagaaatgaattttatgaaatatcacagcttctaagcgacaagaataattttaaagaactagaggag |
| | | ctattacagctcaaagaaaacatcgacgtattggatggtaccctaaaaaacctatctttaggttcagtaccaagaaatttagggag |
| | | tatagagaaagactaccgtcagtatcgcgatttagagattgatgctaaaagtcttttaaagcagattaataactctgtatactcag |
| | | cgcttgatcattttactgttaaagatgattattcaattgctgagaaagactttcgtagcaaagcagccatattacatgcgaaaata |
| | | agaaaattttccaataaaggacgcaatatattaaaagaagagatgttgcgtttcgaaaagataacaaacaatacaaataaagcttt |
| | | ccatgaaaaaacatctcaatatttatccgatctacaagaaaatagaacttcactcaaaaaaacacttgaaaatttagatcttgctt |
| | | atcagattcaagacattgaaataggtcaaacctacgccccatatattaccgcattagaaagcttaagagaggaaattgatttagaa |
| | | ggcctcgcgatctcttcagtcaacgaaaatacacggttgaagaaacaggtagagcaagtgaatgcactcgctcaacttggaataac |
| | | tgtggagataattggtcatgaaatcgaaggtttcgatatgactattgagcgaggtataaatagactgtcatcaacaaacctcgatg |
| | | aatatcagaaaaatgctttatcaagtattacccaagcacatcaatcattaagcgattcttggcgttttttaagcccattaaaatta |
| | | tcaggagataaggtaagagctttcttgagtggaaaagatatttttgattatgttaatcattttttcaacagtaaatttgaaaaaga |
| | | ttcaattgaattttcttgctctactaatttcctagatatttcattatatgatcaaccagccagaatttatcctgtgtttattaatt |
| | | tagtaaacaactcacgatattgggttaaagaaactaaagaagagcgtcgaattattaggttagatgtacttgatggtttgatatat |
| | | gttagtgataatgggccaggggttgatcctgatgacgtgtccgaacttttcactatatttttctccaagaaacaaagaggtggtcg |
| | | cggggttggcctttatctctgcaaacaaaatttagcggtgagtggccatagtattttctacgaaacaagaacagagaaaaaaatac |
| | | taaatggtgctaattttgtaattaatttcaaaggaattaaaaatgcttgataattctactttcgattacaaaccacatttaaaatc |
| | | tgcttatattgatccgattagaactgtgacagtcatcgatgatgaatacccaactattgatgatttaatttcaccgaccaaagaca |
| | | gtttttctcaagacaacatttctcgattaaaagatattattgatataagtcgaagtgaagaatataattggcttttagatgtctat |
| | | aatggaaaagagaagaaaattcaagagggaaccgtatctaaccgtctttatcacagtgatctactaatcttggactatcatttaga |
| | | tggagaggactctggatattgtaaaaaatctatagatattattaaaaatctatctgaaaatcgtcattttaatattgttgcagtgc |
| | | atactaaaggttatgatggacaaaagggttcagttaatgaggtactaatcgatattattacttccttacaggaaagacccgctatt |
| | | agtattttaaatgataaaatcaaatctagaatagatgatgctttagatgaatgggaaatcgaagatccaagtatcagggaagatct |
| | | aattaattcagtttctacattagatttacttttcttgattaataaattcgggtcaaatttaagttcaggatgtttcgactacgaag |
| | | ttcttgatgtttttcataatatatttgatcaaaaaccagacaatataaacatatccaaaatattgatttttaaatggatctcatca |
| | | gaaaagttacatagatacgctgaccaatttaataataagacatcaaagttctttgattgggggacaaatgaaaaccacaattggat |
| | | aaaaacagaagacttatttattactgtccttggtaaaaaagacacaccaatcagtgacataccgaatcaacttttggaggctttgt |
| | | caaactctaaaccacatccgcacaaacttattttatcaaaactcagaagtgaaattgaaagtaatggtagctatgctgcaagtaat |
| | | ataattaacaaaaaattcttacaggcggcgtggctaaaggaattacttcaaaaagaggatgaatatgctatcaaaacagctgcatg |
| | | gcaagcagtaactaaattgtgggaagaattagcatacgaaataaaacagagtcttgatgattttacaattaatcttgtccgcgact |
| | | taaagaaaattaactcacctttaaactatttcatagagaaatctacacttgatgctgaacttgaacaaattaaacatgcaaattgt |
| | | ttcagttgttcaaaaaaaataactgctcatcatttggttacggggcatgttttggagttcaataataatcactggttgtgtctaac |
| | | tcctatgtgtgaccttgttcctggtcagaaaaacggaaatagtttactccctgttacgctcgtgaaaatgtatgatgcgaaagttg |
| | | ctttaaataatacacgtaaaaatatgcaaaacgagcttaaactacccaatttgccagaaatcaacgaagatgaatcaattagacaa |
| | | atactaaattattccacacagaataatctattgttcgttcagtctgaacatgacgggaaaatacatattcttagtttcaccgttgg |
| | | actcgatggcaaggcaaatcctaaagcaatggattgctatgtggaaaatcaaggtattttctctgaagataaaataatagcactaa |
| | | aatatgccaagcccactgaaaatgaaatgaacataatatccgtagaagcaaaaatagttgctgaattacgctacgaatatgctttg |
| | | aatttattaggtagactcggtgtatcaaaatctcgagtcggattagattttatcaactaaggtgcgttagcacgcacctagtctga |
| | | caggtaccagttgtttatataggtatctgtcagactacatcctctttaggtttctctcgcccagataattttttccatcaagtgac |
| | | attttcattgatgtctaactctcagacattaaagtgtctaacttccttattaatgtcacaagcaacaattgaatttcaccgctttt |
| | | gcgagcatgatcgcaataatatcagcccgttacccggttaattcctatgacatcactcgaaacactgcaatcggctatctctaacg |
| | | tctctgtatggcgtcagggtgatgtatgcgcgccgcataaaccgttgctgctgctgtatgtgttgtcacagtacaaagcaggccac |
| | | ccgcgcctgtttaactacggcctagagatccacgaaccactcactcgcctgctaaaagagtttggccccaagcgacgcactgacta |
| | | tcccaatatgcctttctggcgactcagaactgacggcttctgggaaattgctaatgcggaaggctgcaaaccccgtagaggcaaca |
| | | cccagccgacaaagaaagagctgattgataatcaggtagcggggggttttgatgaaacagcttaccagcaactgcttgcacaccct |
| | | gaagtaattgaccaactggcccagcagatcctgatggatcgtttccccgagagtattcagcggatcctcgccaaccaactgggtct |
| | | ggattttatcgaccgttcaaagagccgcgatccgcgtttcagggatatcgtgcttcgggcttaccattcgcgatgtgctttctgcg |
| | | gttacgatctacgactcgatggtgcgctggttggtattgaagccgcccatattcactggaaaacctatggcgggccgtgtgtggta |
| | | aacaacggtctggcgctatgttcgctgcaccacgatgcttttgatatgggcgcattcgggctggatgaaaaccttaccatccgcat |
| | | ctccggcggcgtcagccgtagcccggtggtggataacctgttctggcaacggaacggccagcagttacaccttcctcacgacaaat |
| | | cgctgtggcccactgaacaatacgtcggctggcatcgtaaacagatcttcaaagcctgagaccgtgagcttcgcaggtatcatcga |
| | | ttgcccaaactgctttatcccctacaacggataaattgcttttaacccctatagcggataaatccagcacaccagtgttggacttc |
| | | agaataacgaatccaaactctagccctgagacaccaggctcttgattattattgataccgtattaatctgtacgaagtttgacccg |
| | | c (SEQ ID NO: 67) |
| 71 | pLG073 | gtaacaccgttgaacgtcggctgggtgttgttcataatccctttaaaaggtctggggatggccatgacctcagggcggtagcgtga |
| | | ccaaagttcatatccataccaattatttttatttaaaatatcaacttattcgagttgttttatttagttcaaagaaggtatcaaat |
| | | tgatagttatagattttttttgtggctgtggtggagccagtgaagggctacgtcaggctggctttgatatcgagcttggattagat |
| | | attgaccaacaagcatcagaaacatttaaagctaatttccctgatgcaaaattcatccaagatgatattaggaaaatcgaacctca |
| | | agatatctccgacatcattgatattaaagctaaacggcctttgttactgagtgcatgtgcaccatgtcaaccattttcgcaacaga |
| | | ataaaaataaaactagtgacgactcaaggagaaatctactaaatgaaactcatcgttttattagagaacttcttcctgaatatatt |
| | | atgcttgaaaatgttcctggaatgcaaaaaattgatgaagaaaaagaaggcccatttcaggagtttattaagctacttaaagagtt |
| | | agagtataactatatatcttttatagccaatgctgagaactatgggattccccaaagaagaaaaagactcgtgctcttagctagtc |
| | | gagtaggtaaagttaccctaccagagataacccatggtaaaaataaaatcccattcaaaactgtacgagattatatccaggacttc |
| | | acaaagttatgttcaggagaaaccgaccccaaagatcctttacatagggctggaacactgagccctcttaacctaaaaagaattat |
| | | gcacactccagaaggaggggatagaagaaattggccagaagagttagttaataaatgccataaaaattatgatggccacacagata |
| | | cttatggaagaatgagttgggataagcctgcgcctacacttacgacgaaatgtaatagttactccaatggtcgttttgggcatcct |
| | | gaccccactcaacatagagcaattagcataagagaagcatcaagattacaaacatttcctttaagctatgtttttaaaggttcgct |
| | | gaattcaatggcaaagcaaatcggcaatgctgtaccttgcgaactcgctagactatttgggctacatctcatagaaaattgtacta |
| | | ataaggattcatagatatatggctaaaataagaacaaaggctcgagctttggacatgcttggcagacaacaaattgcaggtatacc |
| | | tactgccttgagtgagttatttaaaaatgctcatgatgcctatgctgataatgtcgaagttgatttttttaggaaagaaaatcttc |
| | | ttatcttgagagatgatggattaggtatgacaaccgatgaatttgaagagaggtggttgactattggaacctccagcaaattaatc |
| | | gacgatgatgcaattaataaaccagcagtggatagtaataaagcctttcgccctatcatgggagagaaaggaataggccgtttatc |
| | | tatcgcagcaattggaccacaggtgctggttcttactagggccaaaagagacaatgagcttaagccattagttgctgcatttgtta |
| | | attggagtttatttgctataccatcacttgatcttgatgatatagaaataccaattagaactattatcaacgacgaatgcttcact |
| | | aaaaaaactcttgatgagatgattgagcaagcaagaaataatttagactctttatcacacaaaatatcaaaatcaaaagtatcaca |
| | | aataaatacacaattatcatcttttgaatttgatcctattctatgggaaaaaaaattaggtgggctaagactatctggagatgggc |
| | | atggaactcacttcataataatgcctaccgaagaaatattaatagatgacatttccacgagcgatagcaataaaacatcagagcag |
| | | tcttctcgcttagaaaaagctttattaggttttacaaacacaatgtacagtgattcaaaccctcctattatagctcgttttagaga |
| | | ctatctggaagatggtgagtgcattgacagaattagcgaatcaattttttttacaccgcaagaattcaatcttgcagatcaccaca |
| | | ttgaaggatggttcaatgaatttggtcaattcagtggaactgtttctgtttatggtgaagagccaattcatcatgtcgtgacttgg |
| | | aaaaataataatcaattaacccaatgcggtccatttaaaataaaattagcgtatattcatggtcggcttcgtgattcacgcttacc |
| | | catggagttgtgggcccctctgaaggagaaaacagatagatatggtggtttatatatctatcgagatggattaagaattttgccct |
| | | atggagattcagatacggattttctaaaaatagaaaagagaagaacgttatccgcttctgaatattttttctcatatcgacgtttg |
| | | tttggagcaatagaattaacaaaagaaaacaatgcttcattagttgaaaaagctgggcgagaaggattcattgaaaataagccata |
| | | taaacagtttaaagaaatgcttgaaaatttcttcatcgaaatcgcaagagatttctttaaggacgatggcgatatgtctgaattat |
| | | ttgttgagacaaagcaacgtagaaatgaagaacatgatttgttatctaaaagatctaaacaaactaaagctaaaaaagatagatta |
| | | aagaaagatctgtatgatttttttgataagttagataatgattactggaatattgaaataaataagctaatcaataaaaacgagga |
| | | atatttctccagtacagaaataacagacaccaatatagattatgtatacaataaaattaaagaacaaaatgatgctatcattaaaa |
| | | atctacgtaattctgtggatataaagaaaccctctggagttggattaacaaaagagttatctaatttatgggatagatatcaaata |
| | | gaaagacaaaaaatactgttatcactaaatgagctaaaagataacgttgatagaaagcttatagaactggataataaaaataatga |
| | | ttttctcaacttacggaagagacttgaagattctttgaatctacaacaaagttactatgaaaaagaactaacaaagttatataatg |
| | | acgctaaaaatgctttgaaagatgtgcaatctaaagcaaataggttaatttctgataataagaaaaaacataagagtgaactaaaa |
| | | aacatttcttatgaattccaatcaactaatctcaatggcaaagatactgcgtatatattggatgtaaaaagaaatctagaaagtaa |
| | | aattgagaatacttcaaacgaagtgattaatgaaataagaaaactaaccgaccagattgcaataattagtgatagtaccacttctg |
| | | aaaatttatcatcggctcaagtaactgaagcaatcgaaactgaacttgaacatttacgagaccaacaagcaaataacgcagagtta |
| | | atactacttggcatggctctttctgtagtacatcatgaatttaatggtaatattagggcaattagaagtgcgctaagggaattaaa |
| | | agcatgggctgacagaaatcctaagcttgatattatataccaaaaaatcagaactagttttgatcacttagatggttatttaaaaa |
| | | cctttacaccattgacaagacgtttaagtcgctctaaaaccaatataactggaactgccattttagaatttatcagagatgtattc |
| | | gatgatcgtcttgagaaagaaggaattgaattattcactacctcaaagtttgttaatcaagaaattgtaacttacacatcaaccat |
| | | ttaccctgtctttataaatctaattgataacgcaatatactggcttgggaaaacaactggagaaaaaagacttatacttgatgcta |
| | | ctgaaacaggatttgttattggtgatactggtcccggtgtttcaactagagatcgagatataatatttgatatgggatttacacga |
| | | aaaacaggagggcgtggaatgggattattcatttccaaagagtgtttatctcgagatggatttactataagattggatgattacac |
| | | tcctgaacagggtgctttctttattattgagccatcagaagaaacaagtgaatagcggatataaataaatgacaagctctactgat |
| | | tttcataaactttctgaagactgcgttcgccgttttttacattctgtagttgctgtagatgacaatatgtcttttggagctggtag |
| | | tgatactttccctacagacgaagatattaatgctttagttgatcccgacgatgatcctacaccaataataacagcatcagcatccc |
| | | caaggatagaatcaactaaatcaaaagcaaaggtaaaaaaccatccttttgattaccaagctctagcagaagctttcgccaaagat |
| | | ggtattgcttgttgcggattattagctaagagttttaatgttgaagaaagagatataattacagcatcatcccacaaggcagatat |
| | | aacaatacttgactgggatatgcaaagcgatagtgggcaatttgctattgaaataataaaatcgataatcgtttcagatataaatt |
| | | ctggaggacgtttacgtcttctttctatttatactggtgaacatgttactgctgttataactaagttgaacaatgagttaaagaaa |
| | | acataccgtagcgtaataaaaaatgatgatagtatttttattgaagataactatgcactcgaacaatggtgtatagttgttattag |
| | | taaagacgtttatgaaaaagatcttccaaatgtgttaataaaaaaattcactaaccttacagctgggttgctatccaacgccgcac |
| | | tctcttgcatttctgaaataagagaaaaaacccatgggatattaacaaaatataataataaattagacactgcatatgtttcccac |
| | | atcttaaatttaataaaatccaaggagtcaagggcatatgcttatgaaaatgctcatgattatgcagtagatttaatttctgaaga |
| | | aataagatcaatattgcaaataagtgaaaacttaaagaaatctctaagcaaaaactccttatcccattggcctatttttcactatg |
| | | caaaaaatggttgtaagaattttctattaactggaaaaaaacaaaaagacttatcagtagaacatctaaggaatatactctctgct |
| | | gattctttagaagaaattcaacacgctattgaacacgcatctttaggtaaaaaggaatacttaagccaagatggtgaagaagataa |
| | | aaagttaatgcaattatgctctctggaaatcacgcgcaggagtttaagatatcattctcatatagataatgtgtccttaaaacaag |
| | | gaactttacttttagatgcatataattttgtctatctatgcatacaaccattatgtgatagcgtcagattgcatgaaaaagccgat |
| | | tttttattcctcaggggaacactggacgataataattacaatttgttaatcgaagatgaatatggcggtttttataaaattaaaat |
| | | gccggcaaaagcttctaatattatttcattttcatttggagtcgaaaatggaaacggtgtcatcatagggaaaaagaacaatctag |
| | | ttaatactgactatatctcattcgttcctttactcgttgaaaaaatatctactccaaaagtattgaaatggatcggggaaataaaa |
| | | acaacgtacgcgcaaaaaataacaactgatattgttgctaatctgtcaagaataggtttagatcaacatgagtggttacgaataaa |
| | | atcaaaagatatataaatgattatatatgccgtcgttttataaaaactggcggcatgtatatctagttagtccatcatagaagtca |
| | | agaaatttagtttgccctatatcttatagaaaatatattttatatgcttaaaaaacaccatctttataagatggcatttatgtgct |
| | | ttgtttcgatcaattacaactg(SEQ ID NO: 68) |
| 72 | pLG074 | gattattatccagcctttgcgcaggagagggcatgaactgctcactctgatagccgctcttgccatagttgagcttactccacaaa |
| | | agtagacacattctgttcttacctagacgcctgctcaaaggcggccgggatgactatagcggtgatccagattgtacctgatccct |
| | | atacatgatttgtatcattgtcaagctttttgaacgatttaatctcttattggagttcatgatagccacttgaatttcgaaaataa |
| | | ggtactatatctagtaaagtcttagtcaatttttggtatatacagtggaagtggaaccatttcgtgtcctttgtttagatggcggt |
| | | ggaatgcgtggcgtgtatcaggcgacgtatctcaatacatttgcacagcgtctgcataactctggtgaaggagtcttagatccagg |
| | | aaaggcatttgatttaattgtgggaaccagtacgggaggcatagttgcctgtgcgctagctgcgggggtctcacttgaaaaggttc |
| | | ttgcactttatcaagtgcatggcggaaaaatattccctcggcaacgattacgtgcactacctcgagtggggaagtatgtccgtggc |
| | | ctattttctggtcttgcgtctggcgaccaggctctgcgagcagtcctttctgattcattcggtaccgaaactatggggcaggtcta |
| | | tattcgtcgtggaattggtttagccatcactacagtggatctgaataggcatgctgccacagtttttaaaacccctcatatgagtc |
| | | gtcttaatggacgtgacaacgatcgactattagtcgatgcctgtatggcgactagcgccgcccctatcctgagatcaatagctcgt |
| | | ctaactgaacctggcggtggagccactgttgattatgttgatggcggtctctgggcaaataatccgggggctgtcggcatgataga |
| | | agctcatgaaatccttcagcagagaggagagattgaacgtccgattcatttatttatgctcggtacgcttccattgcaaggaggtg |
| | | aagaacttaagagcgcagataaattacatcgaggtgttttggggtggggagcagggattaaggccatcacagtaagtatgaattca |
| | | caggcagttgcgtacgactacttggctcggaaaatcgcagaattgcgaggatatggaagttttgcatatcgactcccagcacaatg |
| | | cccatcaggagaactccagaaatatttggaaaatatggacgatgcacgtcctagggtgcttaatgcgcttgcccgacaagccgtct |
| | | cagatgttgattacgcttgggctacggcagaatcagtaagtaaaatgggcgcgtttcgaactgcattggcaagttcgtccaattat |
| | | agttgtcataaatccgaggaacaccatgaccattattgattgtaataaagagatgagagggtatcactcagaagaggtaaacctct |
| | | cgaatgcagagcaggcagaaatgcgcggccgccgcgacaatggtcgaacaaggctccgaaacggattgacaaaggctggtcatcct |
| | | ttgccgaaggagttcagttctcaaggctcttatgcgatgcgaacaatggtccaggatgatgcatgtgactacgatattgatgatgg |
| | | cgcgtatttcgataaagaagaccttaagaactctgaaggcgattatcttagtgcgctagatgttcgtaagcgggttcggaaagcat |
| | | tgaaagacgaccgattggcatatgatgcggttgtcaaaaccaattgtgtgcgtcaaatgtatcccgatggatatcacattgatatc |
| | | cccatttatcgtacgacctgttctaaagatatttgggataatgacatcatagagtatgaattagcaagtggcgacgaatggaccaa |
| | | atcagatgcacgtaaggtaacgagttggtacaacgatgcggttggtaatgaactgaaagcgggggaatctgataccagtcagatac |
| | | gcaggatcaccaaacttactaagaaaatggctaggagccgtaatacctggaaaaaaaagacaaccagtggcatttgtatttcgaag |
| | | ttagttgtagacaatttcgttgcgcgctcaaatcgtgatgatgatgctttgcgtgatacctggaaggcaatcaaattgcagttaga |
| | | agtcagtcaacgtattacccacccggtgtttacggacaaaaatcttgctgaggaaggagacgaatgcgttatttttttccgggaat |
| | | gtttgggtgaggtgctggaaacattaaaggtgctcgacgagcatgactgcacaagtaagaaggctggcgacgcttgggatgaggtg |
| | | tttaatacaacttattttagcgcccagtgtaccacggataacactacatctaaatcgctgctacggcctgcagttgcggccactgc |
| | | tagcctgtctttccctagttatcccgtacaacctaacaaatcatcggggtttgcctgatgaagtgggctatagacgatcccgtgcg |
| | | tttcctgagggagaaggatgaactcacacatcttgaaaccgagacgggttggctaagcacggcttggcgtatatctgaagagggct |
| | | cgatcaccgttgatatcgacatgtttatccatgggcgattgtttgctggggaaatgacatatccggacgcgtttccggattctccg |
| | | ccctacatacgtccgcgagataaatcagagcgatggactaaccatcaatatggcgtgggtggttcactgtgcttgcagtggcgggc |
| | | agataactggcatagtaatgtgactggtgcagatatggtacgcagtgcgcacgagttgctgagtacagaacagcatcctgaattac |
| | | ctcattctgttccctctgcgcatcgcttgacggaggggcaaaaccttaatttcgtatttcgacgttatgtccctacctccgaagtc |
| | | gaaaacatatttactatgctcccacttcagtctagaacccgaatatcatcttcaactgtgtataacgaagggtcggcggtaatgtt |
| | | cacagccagagtcgctgacgaacaggatgagcttcgaaatgttaccgatatccctcaagggctcatcgattttgttagtattttgt |
| | | cgttgtcctatgagggctgggtctttagaagcgactactttagccagaggcaatccttagaatctgtagaagcattaatccagata |
| | | ttgatgatggccggttttaacaccgatgacattctggttaaggaaggggataagttcaaggctaggacgatcatattattaggcaa |
| | | ggaatggtcatcactgcgagtattcctgttagattctggggagcaaccagtgctgcgggagcatcgagttgttagatctccgaact |
| | | caaccttaagactttcggaagaatcacagaagttgagtaagatccgcgtaggaattgttggactgggatccgtaggtagcaaaatt |
| | | gcaatttcacttgctcgttcaggtgtcagacaattcttattagtcgatgacgactatctcacgcctggcaacttggtgcgtcatga |
| | | gttggggtgggcccatgtgggagctcataaggcacgggccgtaagcaatactttagcgcttatagcggctggtgtgaaagtggatg |
| | | taaagactatgcgtcttgcggggcaggaatcggcggtgacagcagcggctgcactaaaggatctgtctaattgcgacttgttgatc |
| | | gatgctacagctaatccagaagtttttttgctgttagctgcgactgcccagcgaaatggaataccgatgtgctggggggagatatt |
| | | cgcaggtggttacggaggcatgatcgctcgagcacgtcctaaacacgacccaaatccattagctgtgcgtgacgcttaccattctt |
| | | atctctcaaccctccctgaagcaccatttaagaatatggctagctatgatgggagtgatgaacaaccacttatagcatacgacagc |
| | | gatgtgggctttattactactgcactgacacggttggctgtggatactgctctatgcagagagccaagcgaatttccgtactcttt |
| | | gtacttgctgggtatgcgacgtgaatggattttcgaggagccatttgacacacggccagtcgaaataagtggagaaggctgggaac |
| | | gcgacgaaaatgctgtgagagatgaagatagggtcgcagttgcaaaggcattggtaaatatgtttcaaggaaaacaaagtgctaac |
| | | actgatcctacctcctaagcagcatgagttaatgatgactgcactccaaaatgctggtcaacgcgaagtcggcgggattcttatgg |
| | | gtgaacatgtcgggacaaatactttcatcgtccgggagataactatacatcgccgtggtacgtttgcttcctttgtacgacgtatt |
| | | gaggatgctattggtgggctccgtgttttttttaaaggaactggatacgattatgttcgcttcaattatatcggtgagtggcattc |
| | | tcacccttcatttgagccatacccaagcagaacagacgatctgtctatgttacagattgtaaaggatgaaaccgttggtgcaaatt |
| | | ttgtggctttgttgataatcaagctcggacctgatggaaaaatggtttcaacagtccatacatatcttcccgatggttcgaagatt |
| | | ctctcaactcttaagattcagccttaactcagaatgtcagattgtgaaattcatcttctagaggctaattgaagcatgctgattat |
| | | tttttgaggcggaagtatgttgcct (SEQ ID NO: 69) |
| 73 | pLG075 | aactcacccgctctgaacgagccccttgaaacacaagacaccgtttttcccttaccataagggataggcaaacgactgtgtttatg |
| | | actaccagcagagacaaaaccatcgaagtgctcggccacccatttgcgcctctaggttgctacgagactgcagaggatccatgtag |
| | | cagattacctcggccatgaagctgctaacggaagcgaagccatagaccgtaggcgatacacgtacgtatggctttccggaagggcg |
| | | atcctagtcaactgtctgatgtccgccaaatctttctcaatactggtcattcaccttttccttgaccggctgtcaggcccaacgtg |
| | | cattcagatcgtcgcctaaatttgttgcatcacgtagagtctgccgcgtgctcgcccctatgccagactagtctgatgtggcggat |
| | | gagataggtcacgacggtggtggctcggtagagtcggcatcgccgagtcaacgatggaacgtaaggggcgtgaatgcaaatcagcc |
| | | gtaagctcaacctttatgagatcgaggatctctaccagtcgcttggtacggattccaatctcaggcttcctatcagcatgagccac |
| | | ggcggggggttgggcgtggatgcttcgctggcccagttcatcgtcacctgggcacgtgcttgcgaaaaaaccgtccttcacctata |
| | | tgcccccgctggcgacgacgccatgacgcaaatcacgcagttggcgcagagtgcttctgggttcttcgcgctgatcatgtgcagtg |
| | | aagtccacgctcagaatcatcaactgatcgatcggcgggaagcgcttctggcgatcaggccccttgtcgatgcgatgttcgcaggc |
| | | gaccttcgtaacacctccaacatccgaggcgcccgtccaacggccatcaatctgttctgcgtgaacaacgcaaagcgtgagttcat |
| | | caagccgttttacttcgatcacgccgtgccgaaagtccagccgagatcttggttctcgactctcttggagacgtcatcgaagctga |
| | | tgaatgctcgcagtggacaaggggcactgcttaggtcaggtctcccggcattgggcagcgtgctttgggagttgatctccaacgct |
| | | gaccagcacgctgtcactgatgtaggcgggaacaagtacaagaaggcgctgcgtggcacctccatcaaactcaaccgaatgagtcg |
| | | tcaggatgcgctgatgtattcagaccaagagccggagttggcgcgctttatcctgaagcatttcctgagagctgaggtactggact |
| | | tcctggaagtctcggtcatcgacagcggtcctggactggcacggcggtggctgacggcgaaggaggggcggccagtagaaagcctg |
| | | gaggagctgagtcttgaggctgagcttgaggccacgctcgattgcttcaaaaagcacattacatccaagccgcagtctccgaactc |
| | | gggtatggggctgcataacgctgttcaagcactcaacaagctcaaggcgttcgtacgcgttcggacgggtcggctttcactgcatc |
| | | aggcttttcagggaagtgatgagattatggagttcgatccgtcgattcgatacggtggccgtgtgttggccgctgtggaaggcact |
| | | gtcttcaccatctgcattccggtgagctgacatgttcgatctcatggattttgaagtcgagttgcgtcagtcaggtaagccggttc |
| | | atgtggtggttttcttcactggccctgatctcctcacagacacgcaagcggctcacgctctacagcaccaattgtcgggttacgtc |
| | | atgcctgacctagtggtgtttctgatgcctggttacaccttggatgaattccgagcacaccaggcaaatgctacatcgcccctgat |
| | | ggcggagctaagccgtaaaggcccaggctcgcctcgcacctacgcgagtgcgttctatgacgtgaatggtgccattaccgagtacg |
| | | tcaatatctctggccctgaggagcagttcgaggaactcatcaagcacaactctaacgctatcgcgaggactggcctgacccacctc |
| | | gtcgaacgctccaacgtgctgaagaaggcgcctgcaggcttcttctactcaaagccctcttctcgggcttcgaactatttcattcg |
| | | ggcggaagacctgctctctgagaccttgcatgcccactacctggcgtttgcatgcctatctctcatcagtaaggcaacggaagatg |
| | | ggatggggacgcccgataccctgtatctggacacaatcgcattgctgcctctggcgctgtccatgcaggtgtacctcatgcgattt |
| | | gagcagccgggctttgcgaatatccggtcattccattcgcacgaaggcctaatcaagggtgggcctttgcccaaggcagtttccgc |
| | | cctgtgtctcatttccgcatcgacccagtgcggcctcgcgcagcaatgggtgaaggtaaacagtgctccgccgacgcgcgtggcca |
| | | ccattctttcatttgagcgctcatcggactcctgctccgtcttgcacacactgaagcagcccgaagactttgaaatgttgggggag |
| | | ggtgaagcgagcgggattcgtctaattcggatccatggcgagcggttcgttgctgagcacagtgaaaccaagctgctgaacatcgg |
| | | cactgatcatgcgccgcccctgctgcaatccaagttctactcgttcatgggggccaacctgttcagctgcttcacccatgaccggc |
| | | caggactgaggcctcggacagtgcatgtcgataaagataacctggtggctgccagcgatttcggtgaatggttcgacagggtactg |
| | | cttgaggaagctgtcgcgtcgacccgttggatcatccacgatgacgacgctgccagtgcggccctggccgatcgagcgatcgctta |
| | | cttagggatgtgtggcgtcaaggtcggtaacaaggtctccttcgatgacttcgatgccaacacgaattttgacgggtctgtcatcg |
| | | tcattgccgctgctgccgaacgtggctcacgcctgcagagtgtgagccgacgcctgcgtaccgctcagcaatcgggtaccaggctt |
| | | tacattacgggggcactcttcgggcgcagctatcaactgatgaaggatctgcagagcaacctgacgcaacctgccaaggatcacag |
| | | ccggtatgttttcaagacgtacatggagatcccggcagcggagcttgcctgcacgagtcattgggccgaagagcagcggctgctca |
| | | tctccttgcattcatttgcggaaactttctcgccagcgattacgcagcgcatggaagtatttgatcgcgcctctactggggggctt |
| | | ggtctgaacccattttggccgagcagtcacaccgggcagccgatgacacttagccgaggctttgcgtttgtcgacggtacgaagga |
| | | tgtgaggggcgcgacgtcaacggatatttacctaaccatcttgtggattctgcagaatgcccggtacagcggtaaggtgcagaacg |
| | | ccaagcggcttgagtccggtgagcttcagcaggtgctcctatcgccggatgtgttctcgcgcttcgacgatggcgttatccaggcc |
| | | gcattcttgcgcgcagcggtgccggcggagcttgactacagggctcatgaaacccacagcctggccatatcggacatcattcagcg |
| | | catcgccgcagggtacggacatgaacgtggtgaagccgccatggagtttgtcatggccttggctatcgggaagatacgactgcaca |
| | | aggatgtcgataaccggctgcggagtaacttgatcaatatcttgacgccgcacgttcaggagatccgttatctgctggatccgaat |
| | | tacgaatcaccgttgtgatcaatttccgctaacccgttgcatgcgaggtatccagttaccggcaactcagctcatggctgagctga |
| | | accctggttgctcttctagtttcgatggcttgccgattgccgggatcacccacctgcgtcggttctgcgacgaaggtctaagggca |
| | | gggtggtggcacctggcttgctcattccgtttgacctcgccaccat (SEQ ID NO: 70) |
| 74 | pLG076 | cgctcagtccggttggtggttttggttggtttggcgattgctcagatcgcacaatccgggctgagttccctttcagtgatctacta |
| | | ttccgcgcagctatttagtggatataatcacgctttgaaaaaaaaacgggtcaattactcttcgccccacagcaacgaataaggag |
| | | aaatttgtgagtaacgtcaacactttccttaaggaaaatttatcttcagtaagtaagaatgtttttgtggctcctggcatccctga |
| | | aaaaaaactgaataatgtcgctaaagcatttaatgttgtggataacttgaatactgtgctagccatttatgacaatacggtatttg |
| | | gtagcgcaaaagatggcatcgtttttaccggtgaaaaactggtcataaaagaagcttttgaaagtccttatgacttgttctacagc |
| | | aatattgaagcagtagaatatatagaagatgtcacggtaaatgataaaggcaaggagaagcgaacagagtctgtttccctcaaact |
| | | aaaaaatggcgaggtaaaacgaatcaaaggcttgatggagtgcaactataagaagttgagcgacattcttaagcataccatcagtg |
| | | actttgatgagttcaaagaagaagatcagctcatcactcttgccgaaatgtcagaagctctcaaagtggcttatgtcaaaatcatt |
| | | gtgaacatggcgttctcagatgatggtcaggttgataaaaaagaatttgccgaaattctcttgttgatgacccgacttgagttaac |
| | | gactgaatcccggtttacactgcgtagttatgtcggttcagaatccagtctgataccggttgaagaattaattgcgatcattgacc |
| | | gggaatgtgtcccaagccataacaaatcaataaaagtctctcttgttaaagacctgattagcattttcatgagtgttaatgaaggt |
| | | gaatataaaaaattcccgtttcttcagcaagtgcaacctttgctgggcgtaactgacgaagaaatagaactcgcagtaatggctat |
| | | tcagcaagattttaagatgttacgggaagatttttccgatgatgcgctgaaacgcagtatgaaagaacttacggcaaaagcaggtg |
| | | cggtaggcgtgccactcgctgctgtctatctctctggctctgtcatcggtatgtccgcagcgggcatcacttctgggcttgcaaca |
| | | cttggacttggtggcgtgctgggtttttcaagtatggcaacaggtatcggtgttgcggtgttattaggtgtaggtgcctataaagg |
| | | gattcgtcatcttacgggtgccaatgaactggataaaaccaagcgccgggaactcatgcttaatgaagtcatcaagcagacacaat |
| | | ccacattgtccgcgctaattaatgatctaaattatatttctggaaagtttaacgacgccctggatgcgcataatcggcaaggagaa |
| | | aaaattctaaaactccagaagatgatgaatgcattgaccggtgcagcagatgaattgaataagaaatctaataaaatgcaaaacag |
| | | tgcactcaaacttaagtgccctgtttatcttgatgaggccaaactcagttcgctgacccgagagcccatcaaaaaacaattccatg |
| | | atgttgttctttcattctacgaagaatatcttgttgaagagcaaaacgatgggaagagtgttgaagtgaaaaaacttaagatcaaa |
| | | gaaaacgcttccactcagcaattagagaaacttgccgcgatctttgaaggcatcggctatttcagagcgggggatgttattaaagg |
| | | caaactaactgggctattctcataatgaaaaaaccagatactcaggtatcggccttgctggtgcagaagcaccagcttgaacaaag |
| | | cgagcatcaattgggtgaccttgatgctgctctagaagcgcttaacgctttgcaaactgataccgaagcttctttagatgaaatga |
| | | ttttggctatggatggtgttctggaacactcaggtatcacgtttgatgaggatatccacacaacggtttctagtgaattcagcgat |
| | | taccttgaatcctgtttgaccacgtcatcgtccagtatcagtaaactgtcgatgatagaaacaatagcgttcaccagcgatatgga |
| | | ctgggaaacctattcccagtccatatcgcagtatgcccataaacacaatatcgatttaatagtcgatccgtttagcgccctgatgt |
| | | ctccaatccaaagaattgctctggaaaaacgtattcaggaagacttgaccttaaagactgcccgctgcgacaaatatgattacatg |
| | | atcgctggcacctgtggcgttattggcggacttatcgatatttttctggtaggcgtacctggagcaggaaaactgacccagcttgc |
| | | agataatgcagtggacggtgccgttgagaaattcgcttcagcctttggatggaagggcagttcagaagcaagcgattcgacaaaaa |
| | | gcgctatcggttttctggagagaaaattcaaaatcaattatgaccatcggcatggcggagatgttgacggtttgttcaggatgaac |
| | | acgaagaatcaccatattaaaagtctcgcccactccccggacttagtcggtttatttttctcgatcctggatcaatttaccagtac |
| | | ggcacattttgtggcagacggaaaattggtttccgtagataccgagacttttgagcttaaagggaataacgttgtctctaaggtat |
| | | ttagtggtttcgtaaactggctgggccaccttttctctgatatggcaggttcttccggtgcagcagggagaggctccggtatcccc |
| | | attcctttcttttcattacttcagtttattaatgtgggtgaatttggccagcatcgccagtctttcgcaaccgtcgccgtccaggt |
| | | ttttgagaaagggtatgacttacggcatggattagcgatggcgatccccgtcatgattactgagttgcttgtgcgaatcacctgga |
| | | cggttaaacaacgttgctatcataagaaggactggggtgaatgtattccttcagcaaataaccctgaactcaggcgaatgttgctt |
| | | gtggcgcatggaaccttgtgtctgatggatgtaggagatgcggcacttcgttcaggaggcgaaatgattcagttcctcctgagaac |
| | | gaacctcatcggctggacgaggtttggaattctagcgattaaagaactccatgtctggtataaagcaggcggaattgatgccaatg |
| | | ctgtagatgaatatatggatcatgaacttcggcgaatgctaaaagcggggtagcgttacggctttgttgaataacattacgtttgg |
| | | gtgcttggctgtaaaaagctaggcaatggcgtatctgtcgacgcaatgcagaaaaggcaacttaattgcgaaacagaaatgttcgg |
| | | tgagttgcttgaccgtcctatggcagctaagtgccagaagtcgacgttgctaacatcagtatgtactcatcggcacagtccatgtc |
| | | agagctattaactatagataaaaattcaataattaataaaataagaaccatctttctaggtggttcttattattaacaataaatat |
| | | tacgatttcaacgagggttagaatg (SEQ ID NO: 71) |
| 75 | pLG077 | cctggtcctgccaattgctcccccagccatatgacataatccttttgaataatagggtttttatgcttgtactctagcccattcgc |
| | | ggtatcattttacgatctctcttccagttttatgcttaccgcctttgcctatcgtagaacaatgccgggaagcgttatcagcgatt |
| | | aagggcaaggaatgggcttctggatatttgttattatgctggcggttatctggcttctgttttccaaaaagaaaaaatcgccgccc |
| | | cccagagtaaacaacaaaatcatcaccaaaataaatcattcatctcgacagaaatctctcaataagccagataacagcatgacaaa |
| | | tatgcattctcaggcctccgatgatgacgaactggcaacctttacttttgtgaacgggcagacggttgaatacagcaccagccgcc |
| | | agccgtcacgagaaaacgccgcccgtagcaataccactccagcgcgatgggtcaaaccgggagaaagcatcaccattcaaaatgtc |
| | | gtcattaatcacggttatttttatttcggcgggcggttaaaaacacattcatcaggagaatatggatatctttataacgatgactc |
| | | cgacgcttcgctggttaatgacgcttttcccatcgagcctggttcacggcattattatgatgagtcactgggatactggcccagct |
| | | ttgccacactctcccctcgctgccgtggcgcctatcttgactggctggcaagcgatcgcagcgatgcgagctgccccgttggctat |
| | | gtttttatctatttttacggtctagaacgccgcgtactggccgatggcacacaagaagccatttctgacgatgaattcaaagcatt |
| | | attcgaagagatatcgcgcctgagaaccgtatttcaggcaagcggttccttccggcattatgcaacgcagttgctggaaatgatga |
| | | tcgttctccgaccgaagttgctttctatatataccgaaaacgaatatttctcatcgaggagttcattactgttcagattaaatcta |
| | | gcgactgtggtcgataaaggacaacctatttgtgccgctctggcactggcatggatatactattttcctgattacaccctgcgcac |
| | | gcctgcccgtcgatgtcatgctgaattttccgcattattcaaacagcgttatactcaaaaatacggtgacggtattgtcgtcaaac |
| | | ccaataaaacacggttgtatttaagctatacccccgccagtggtacgcttcgggaacttcaggtaaaaaaacagatggatcttccc |
| | | gatcccagcgttttaaaagccccagttcagaaattaatttctgttgcagaatcctgtatcaacgcgctggatgcctacagtcgcta |
| | | tctcggtaaaaaagatgcctcaccaagtgatgtcgccgccatcatgctgcttcccgatgaaatactgaccgaagatgcagaacgtc |
| | | tatttgctgaatttaaacactgggcagatgagaaaatccgtgaacattcaggactggcgacagtggctgatttctgggccagactg |
| | | ggtatgcctgtaccggataagattaataagaaagaagccgagctgatgcaaaatttcgcccggcgagcaggctacggcattgcgcc |
| | | ggatatgcgctatcaccttgtcagaccggatccagaaggtcatcttgttttatttcctgaagggcatgcggaattctacgtaccgt |
| | | cggcggaatttacgtcagtctctgtggcgcttcggttgggtgccatgattgcacaaatggacaagcgcgtggatgttgctgaacag |
| | | gccgcgctggagaaaacgattaatcataacgatgcgctgtcgccaacagaaaaacgttcgctgcacgcctacctcacctggcggct |
| | | caatacgcctgcaaatcaggctggtctgaaaggtaaaattgagcaactcagcgataaagataaatccactattggcaacgtgatta |
| | | tcagcgtcgcctgcgcagatggaaaaatcgatccggctgaaatcaaacaactggaaaaaatctacgccagcctcggtctggacagc |
| | | agtgccgttaccagcgatatccaccgactgtcaaccgcagaaacaactccgacagctacgttacaaaccccatcagcgacgagcgg |
| | | cgcgttttctcttgatgaacggatccttgcccgtcatgaatccgacacaacggacgtacgccagttactgaacaccatcttcaccg |
| | | aagatgaacccgcagacgaatccccagcggagatcccgccacacgctggcgcaggtcttgatgaagcacatcatcaactttaccaa |
| | | cgtttgcaggaaaaagaacgctgggcgcgaaacgaagtcgctgagctatgccagcagtttaatttgatgctaagcggcgcgattga |
| | | agcaattaatgactggtctttcgaacaggttgacgccccggtgcttgatgatgacgatgatatttacgttgacctggaaattgcac |
| | | aagaactcaaaggataatttatgtctggcattcgtattcgtctcaaagaaagagacgctattattcagtcactgaagtcaggtgtt |
| | | acgcctaaaattggtattcagcacattcaggttggccgggtcaacgaaataaaagcgctgtatcaggatattgagcgtatcgctga |
| | | tggcggcgcaggattccggctgattattggggaatatggctcaggtaagacattctttttaagcgttgtgcgctcaattgcgctag |
| | | aaaaaaagctggtgacaatcagcgccgatttatccccggacaggcgcatccacgcgacgggtgggcaggcgcgtaacctctactcc |
| | | gagctaatgaaaaatctatccacccgaaataagccggatggaaacgcattattaagcgtggttgagcgctttatcacggaagccag |
| | | aaaagaagcagaaagtacaaatgtgtcagttccgacgattattcaccaaaagctcgccgccctgtctgatatggttggcggttacg |
| | | atttcgccaaagtcattgaatgttactggcagggccacgagcaggataatgagacattgaaatcaaatgccatccgctggctaaga |
| | | ggtgaatacaccacgaaaaccgacgcccgtaacgatctgggtgtgcgcaccattatttctgatgcctctttctacgattcgctaaa |
| | | gctgatgagcctgtttgtccgtcaggccggatacgcgggtctgctggtgaatctggatgagatggtcaatctgtataagctcagta |
| | | acactcaggcccgcgttgccaactatgaacagatactgcgtattctgaatgactgcctgcaagggacggctgaatatatcggtttt |
| | | ttacttggcggtacgccagaattcctgttcgatccgcgcaaggggttgtacagctacgaagcgctccagtcccgactggcggaaaa |
| | | tagcttcgctcagcgggctggtgtcattgattattcgtccccttccctgcacttagccagcctgacgccggaagaactctatattc |
| | | tgttgaaaaaccttcgtcacgtttattccggcggcgatgcggataagtatctggttcctgatgatgctctgacggcatttttacgc |
| | | cactgtagcaacactattggcgatgcctatttccgtacgccacgaaacacgattaaagccttcctggatatgctggccgtgctgga |
| | | acaaaacccatccattcagtggtcacagttaatcgccggtgtcgcgatcgcggaagaaaaacccagtgatatggatgaaataacat |
| | | cggcagaagatgccgatgaggacggtctggccgacttcagattatgatgaacgaataccagcggctggatccacggatacagaagt |
| | | ggatataccggcagggatgggccgatctcagggaactgcaaaaaaaatccgtttcaccgatattagcgggcgatcgggatgttctg |
| | | atcagcgccgcgactgccgcaggtaaaacagaagcgtttttcctgcccgcctgttctgccattgcggatattcagggcggctttgg |
| | | cattttatacatcagcccgcttaaggccctgattaacgatcagtatcgaaggctggaaaacctcggtgatgcgttggagatgccgg |
| | | tcacgccctggcatggtgatgttgcgcagagcaaaaagctgaaagcaaagaagaatcctgccggtattttgcttatcaccccggaa |
| | | tcgctggaagcgatgctgatccgcaatgcgggatggttaaagcaggctttcgcgccactggcatatatcgccattgatgaattcca |
| | | tgctttcatcggttctgagcggggtatgcagcttctctctctgttaaatcgagtcgatcacctgctgggaagaatcaacaatccag |
| | | tcccccgagtcgcactcagcgcaacgctgggggaactggaacaggtgccgttatctctgcggccaaatcaacgtctgccctgtgac |
| | | attattaccgacagtcagactcacgccacgctaaaagtacaggtgaaaggttatctggaaccgctgaccacctcgggccagcaatc |
| | | tccaccgtcggcagagacgcaaatctgccatgatatctttcgcctctgtcgtggtgattcccatctggtgttcgctaatagtcgca |
| | | aacggaccgaaagcattgccgccacgcttagcgatctcagtgaagcgagcatcgttcccaatgagttctttccccatcacggatct |
| | | ctgtccagagatctgcgtgaaacgctggaacagaggcttcaacaaggcaacttacccaccaccgccatctgtacgatgacgttaga |
| | | gcttggcatcgacatcggtaaagtcagctccgttgtgcaagttaccgccccccattccgtagccagcctgcgtcagcgaatgggac |
| | | gctccggtcggcgcgactcgcctgccgtattgagaatgctgattgccgaacatgaactgacgccaacatcaggcattgtcgaccag |
| | | ctcaggcttcagcttgttcagtcgctggccatgatccgcttacttatcggcaacaaatggtttgagccagctgatacccggcagat |
| | | gcactattccaccctgttccatcagatcctggcgatcgtggcgcagtggggaggcgtgcgtgcggatcagatctggtcacagctat |
| | | gcctgcaagggccatttcagaaagtccggatctatgacttcaaaacgttattgaaacatatgggggagcaccagtttctgacccag |
| | | ctctcaagcggcgaactggttctgggcgtcgagggcgaacgtcaggtaaatcaatacaccttctacgccgtgttcagcacgccgga |
| | | agagtttcgcattgtggcggggagcaaaacactgggctccattcccgttgattccccactgatgcctgatcaacacattattttcg |
| | | gcggtcgacgctggaaggtaaccgatatcgatagtgataaaaaagttatttatgtcgaggcgacaaagggtgggcagccgccgtta |
| | | tttggcggacaagggatgtccattcatgatgtcgtccgccaagaaatgctcactatttatcgggaaggcgactaccgcatcaccgt |
| | | tggcaatcgcaaggccgattttgccgataccacggccaaaaacctgtttgatgaagggctgcactgttttcgcaacaataatctgg |
| | | cttcggaatgttttattcagcagagacagcatgtctacattcttccctggctaggcgatcaaaccgtaaacacgttgtcggcatta |
| | | cttatccaacgcggtttcaaggcgggctcatttgctggtgtggttgaagtagaaaaaactacggtctcggaggttaaacaagcgtt |
| | | attcagcgcacttcaggaagggctaccttacgaatcccgtcttgccgaaagcatcgttgaaaagtgcctcgaaaaatatgatgagt |
| | | atttacccgagacgttgctgacgcaggaatatggattacgtgcttttaatattgaacgcgtgacggagtggttgcaggggcattta |
| | | tattaaggggaagaaga (SEQ ID NO: 72) |
| 76 | pLG078 | cgtgattcagttcgccagactgcagcgttttccatgaatataactccatctggtttagaaagagttccaatctaacgatattggga |
| | | ccagaatcacaggcggcagtggctttacgcttacaataactattctatcctgacaattttaagcctcgtttgttacgatgtaaccc |
| | | tataactatgtggttcctcaaccttttttgcccaaaaaatgcccaatgaagtccaaagtggaaaacagatggttatccgttgatga |
| | | gattgcagattacctcgcgattaagcgagacacggtatacaagtagatcgcaaagaaaggtatacctgcacacatgattggacgcc |
| | | tttggaaatttaaaaaggatgaagtagatggctggatacgcgatggcaaagctggcgaaaacagtaatcaagaataaaaaagcaaa |
| | | tttaggagcagtttaatgaaaaccgtacgtagtgcatgccagttgcaaccgaaggccttggaaatcaatgtcggcgaccagattga |
| | | acagcttgatcaaatcatcaacgacaccaatggccaagagtactttaaaaagaccttcatcactgacggttttaaaactttgctct |
| | | ccaagggtatggcacgcttagccggtaaatcaaacgatactgttttccacctgaagcaagctatgggtggtggtaaaacccacttg |
| | | atggtcggctttggtttattagcaaaagatgctgcccttcgaaatagccacttaggatcaatgccataccaatcagattttggctc |
| | | agccaaaatagcagcattcaatggacgcaataatcctcattcctatttctggggtgagatcgctcggcagctaggtcgagagggtg |
| | | tattcagggagtactgggaatccggagccaaagctcccgatgaacaagcatggataaatatttttgatggtgaggaacccatccta |
| | | atcttgttggatgaaatgccaccatacttccactactacagcacccaagtccttgggcaaggaactatagctgatgtagtgacacg |
| | | ggctttttccaatatgttgaccgcagcgcagaagaaaaagaatgtatgtattgtagtttccgatcttgaggcagcttacgatacag |
| | | gaggcaaactgattcagcgtgcattggatgatgctacgcaagaactcggacgcgccgaggtatccattacgccggtaaacctcgaa |
| | | tccaatgaaatctacgagattctgcgtaaacgtttgtttttgtctctgccagacaaaaatgaggtctctgaaattgcgtcgatcta |
| | | tgcatcaagacttgcggaagccgctaaagccaaaaccgtagagcgcagtgcagaagcattggcaaatgacatcgaatctacttacc |
| | | cattccacccaagctttaaaagcatcgttgctttgttcaaagaaaacgaaaagttcaaacaaacccgtggtttgatggagttggtt |
| | | tctagactgcttaaatcggtgtgggaaagcgatgaagaggtgtatttgatcggtgcccaacactttgatctttcgatacacgatgt |
| | | tcgtgagaagctggctgaaatttcagaaatgcgcgatgttatcgcaagagatctttgggactccaccgacagcgctcatgctcaga |
| | | tcattgacctcaataacggcaaccactatgcacaacaggttggtacgctattgctaacagccagcctctccaccgcagtgaactca |
| | | gttaagggcttaaccgagagcgaaatgctggaatgtttgattgatcctaaccatcagggtagtgactaccgaaacgcattcactga |
| | | acttgctaaatcagcttggtatttgcatcaaacacaagaagggcgcaattacttcagtcaccaagaaaatctcaccaaaaagcttc |
| | | agggatatgccgacaaagcacctcaaaataaggttgatgaattaattcgtcaccgactagaggaaatgtatagaccagtcacgaaa |
| | | gaagcatacgaaaaagtactaccactccctgaaatggatgaagcacaggccacactgaggagtggtcgtgccctgttaataatcag |
| | | cccagatggcaaaacaccacctggtgtagtcggcaacttctttaagggcttggtaaacaaaaacaacattctggtattaacgggcg |
| | | ataaatcctctattgccagtatagaaaaggctgcacgccatgtttatgctgttaccaaggcagacaacgaaattacagcatcacat |
| | | ccgcagcgcaaagagttggatgagaagaaagcacagtatgagcaggacttccaaactacagtgctctctgtattcgataagctcct |
| | | gttccccggtaacaatcgaggtgaagacgttttacggcctaaagcgctggatagcacctatccatccaacgaaccatacaacggtg |
| | | aacgccaagtcgtgaagactctcacgtccgaccccatcaagctttacacccagattaacgaaaatttcgacgcactgagagcccga |
| | | gcagagtcattgctgttcggtactttggatgaggcaagaaagacagatttgctcgataagatgaagcaaaaaacacagatgccttg |
| | | gttgccaagccgtggcttcgatcaactcgctatcgaggcataccagcgaggtgtatgggaggatttaggcaatggctatattacga |
| | | aaaagcccaagccaaaaaccactgaggtaatcatcagcgaggactcatcaccggatgatgccggcaccgttcgtcttaaaatcggc |
| | | gtggctaatgcaggtaacagcccacgcattcattatgctgaagatgacgaagttaccgaaagcagcccagtacttagtgataacac |
| | | gctagcaaccaaagcattgcgagtgcagtttttggcagtagaccctaccggtaaaaaccttactggaaacccaaccacctggaaaa |
| | | atcgactgacattacgcaatcgctttgacgaagtggcgagaacagtcgaattgttcgttgccccccgtggcacaatcaagtacacc |
| | | ctagatggttcagaagcacgtaatggtgaaacctacaccgtgccaatccagctcgctgatcaggaagccactatctatgtctttgc |
| | | tgaatgtgatggcttagaagagaagcgaaatttcacctttgcggcagcaggttctaaagaaataccgatcataaaagataagcccg |
| | | ccactctggtcagcccctcacccaaacgtatggatagctcggcaaaaacctacgagggtttgaaaatcgccaaagagaaaggcatt |
| | | gagttcgagcagattagcttaatggttggatctgcaccaaaggtgattcatatatcgctaggtgagatgaaaatcagcgccgaatt |
| | | cattgaaaccgtattaacgcacttgcaaaccgtgttaagtccagaagcccctgtggtcatgaccttcaaaaaagcctacacacaga |
| | | ctgggcatgatcttgagcaatttgttaagcagcttggcattgaaatcggtaatggcgaggtggaacaacgatgaataaaaccgttg |
| | | attttggggcaccgtcagaattcggtatgcatcacttctatgtggagattcccgcagcgccccgtgacgctgttgtgatctatgaa |
| | | gactatggctttgacggtgaagattctcgccgagaaacagtagagtgtcgcctgatattagccagagagctctggactaagatccg |
| | | cgatgacgttcgccgtgactttaacgctcgcctaaagattaagaaacaaagctccggtacttggtctaccggtaaagtgaagcttg |
| | | accgctttcttggacgtgagttgtgcgttcttggctgggcagcagaacatgcctcacccgatgaatgtctggttatttgccaaaag |
| | | tggctggctttacgcccagaagaaagatggtggctttacagtaaaaccgcagctgaagcaggtcgtgatgatcaaacacaacgagg |
| | | ctggcgtaaagcgctctattgcgcgctatcggatggagccaatatcaaattggaaaccaaaaagaagcccaagtctaaaaagctac |
| | | aagttgaagatgagacccaggatctgtttgggtttatggaaaagggagagttttgatggccttgcaaccgtttgaatggagagaca |
| | | aaccgtctcttattgagcacctgttcccggtacaaaaaatatctgccgagacctttaaagaacgaatggcaagccacggtcagttg |
| | | ctggtgtcgttgggtgctttttggaaaggcagaaaacctctcatcttaaacaaagcgtgcattctgggctcattgttaccagcaac |
| | | tgacaacccgcttgaagatttagaggtatttgagctgttaatgggcatcgactctgagtcaatgcaaaagagaattgaggcttcac |
| | | taccagcatcaaaacaagaaacaatcggcgattacttggtattaccctatgccgaacaaatcaggattgctaagcgcccggaagaa |
| | | attgatgaatctcttttcgtccatatttggaatcgggtcaacaatcatcttggtacttctgctcacacttttgcgcaactagttga |
| | | ggaactaggtgttgcacggtttggccataggccaagagtggcagatgtattttctggttcgggtcaaattccgtttgaggctgctc |
| | | gcttaggttgcgatgtctatgcctctgacttaaacccgatctcctgcatgcttacttggggcgctttgaacgttgttggtgcgagc |
| | | gcgcaaaaaagagtagaaatagacaaagcccaacgggatatcgttaagaaagttcaaaaagagattgatgagcttgacattgagtc |
| | | cgatggccgaggatggcgagcaaaggtattcctatactgcgttgaggtgacctgccctgaatccggttggcgtgtgcctttaattc |
| | | caagtttgattatcagcaatagttttcgagttgttgctgagcttaagcccgttcctgctgagaggcgatatgatattagtatccgt |
| | | gaagtatcgactgatgaggaactggagttctataaatcaggcaccatacaagatggcgaggtaattcactcgccagatggaaaaac |
| | | tcagtatcgcgttaatatcaaaacaattcgcggtgactataaagaaggcaaggagaacctaaacaagctgcgaatgtgggagaaaa |
| | | cagactttgctcctcgtcctgacgatatttttcaggatagattattttgcgttcaatggatgaaaaaaaaacctaaaggatcgcag |
| | | tattactacgaatttcgtactgtaaccaatgacgacttaaaacgcgaaaaaaaggtaatagaacatgtcgcatccaaattagatga |
| | | ctggcagaagcaaggtcttgttcctgatatggttattgaagcgggcgataaaacggatgagccaatcaggacgcgaggctggactc |
| | | attggcaccatttattccatccaaggcagttgctatttttgagcttggtgaacaaatattcactcgcagaaggaaaatttaacttc |
| | | ttgcagtgcatgaatcacttgtccaagctaactcgctggcgaccccaggccggtggtggtggcggttctgcggctacatttgataa |
| | | tcaggcgctcaatactctgtacaactacccagttagagcaacaggatctatcgaaaatatcttggctgctcagcacaaccactgtg |
| | | gaatcagcgagaatgtttcctttgtggttaattcacatccagcgccagagttagatgtggaaaacgacatttatattactgatccc |
| | | ccatatggcgatgctgtcaagtatgaagaaatcacagagttctttattgcctggctgaggaaaaatccgccgaaggaatttgccca |
| | | ctggacttgggatagtcgccgatctcttgcggtaaaaggagaagatgagggtttccgtacaggcatggttgctgcttatcgcaaga |
| | | tggcgcagaagatgccagacaatggtttacaggtgctaatgtttacccatcaaagtggcgctatctgggcagacatggctaatatc |
| | | atttgggcgagcggccttcaagttactgccgcatggtacgtagttactgaaactgactctgcattacgtggtggttctaacgtaaa |
| | | aggcaccatcatcctcattttacgcaagcgccatcaggcattagagaccttccgcgatgatttaggttgggaaatcgaagaagccg |
| | | ttaaagagcaagtcgaatcgttaatcggattggataagaaggttcgttcccaaggcgcggaaggcctctacaccgacgctgacctg |
| | | caaatggctggttacgcagccgcgttgaaagtactgacagcttattcccgtatcgacggtaaagacatggtgactgaagccgaggc |
| | | accacgccaaaaaggcaaaaaaacttttgttgatgagttaattgatttcgccgtgcaaacggcagttcagtttttggtgccggttg |
| | | gcttcgagaaaagcgaatggcagaagcttcaagcggttgaacgcttctatctgaaaatggccgaaatggaacaccagggtgcaaaa |
| | | accttggataactatcagaacttcgccaaggcgttcaaggttcaccattttgatcaattgatgagtgatgcctcaaaggctaactc |
| | | tgctcggctaaagctttctaccgagttcagaagtaccatgatgtcaggtgatgccgaaatgactggcactcctctgcgagcccttc |
| | | tttatgccttatttgagatatcgaaagaagttgaagtagacgatgttcttttgcatctcatggaaaactgcccgaattacctgccc |
| | | aataagcaactgcttgccaaaatggcggattacctggctgaaaagcgtgaaggtctaaaaggtaccaaaacgttcaaccctgagca |
| | | ggaagcaagcagcgcgcgtgtccttgcggaagccattcgaaaccagaggttgtaatctatggcgattaagcgcttttcatcccgca |
| | | cagaaagattagatacggaattcctcgctgaatcgttgaaaggggctgctaagtatttccggattgcgggttatttcaggagctcc |
| | | atctttgagcttgtaggcgaagagattgcaaagattccagaagttaagatcatctgtaattccgagcttgatctggctgacttcca |
| | | ggtagctactggccggaatacagcactcaaagagcgctggaatgaagtggatgtagaagctgaagcgctactgaaaaaggagcgct |
| | | accagattttggatcagctattacattcgggtaatgttgagattcgcgtagtccctagggagcggttattccttcacggcaaagca |
| | | ggctcaattcattatgcagatggcagccgtaaatcttttattggctcagtgaatgaatctaaaagcgcattcgctcacaattatga |
| | | gcttgtttggcaagacgatgatgaagaaagtgcggactgggtagaaagagaattttgggcactctggactgaaggcgtcccgctgc |
| | | ctgatgcgatcttagctgaaatccaccgtgtatctaatcgccgggaagtaaccgttgatgtattgaaaccagaggaagtcccagcg |
| | | gcggccatggcagaagcacctatctaccgtggaggggagcagttacagccctggcaacgctcgtttgtgactatgtttctggaaca |
| | | tagggagatctatggcaaggctcgcctactattggctgacgaggtgggtgttggtaaaacgctatcaatggcaaccagtgcattag |
| | | tcagtgctttactagacgatggacctgttttgattctggcaccttctacactcacgattcagtggcaaattgagatgatggacaag |
| | | ctcggtgtgcctgctgcggtttggtcctcgcagaagaaagtttggctgggtgtagaggggcaaatactctcacctcgaggtgatgc |
| | | ctcctctatcaaaaaatgcccttatcgaattgccattatctctaccggactgattatgcatcagcgggagaagactgactttgtta |
| | | aagaagctggaatgcttctgaagaatcgtttcggtaccgttattctggatgaggcgcataaagcccgtattcgtggaggattagga |
| | | gatcaagcttcagaacctaataatctcatggccttcatgctgcagatcggcaggcgtacacggcatctggtactgggtactgcgac |
| | | acctattcaaaccaacgtacgtgagttatgggatttattgggtattttgaactctggtgctgaatttgtactaggcgatgctctgt |
| | | cgccatggcatgaccatgaacaagcgattccgttgataaccggccagactcaggtgacatctgaggctgaagtttggcattggtta |
| | | agcaaccccctgccgccaagcaatgagcaccatactgttcagcaaattcgtgactacctgtccattgataataagtcctttggata |
| | | ttctcatcgtttcgaagatctcgactatatgattcagagtctttggctctccgaatgcatgacacctagcttctttaaagagaaca |
| | | accctatcctacgccatacagtgctgcgtaagcgtaaacagctggaagatgacggtctgttagagcgtgttggggtgaatacacat |
| | | cccattaagcgcaacctagctcagtatcagtcgcggtttgtggggcttggcattccgaccaatacaccattccaggtcgcttacga |
| | | aaaagcggaagagttcagtaagttgcttcagtcacgcactcgagccgcaggcttcatgaaatctttgatgttgcaacggatctgct |
| | | caagtttcgcatcaggcttaaaaactgctcaaaagatgttgaaacatacggtttctgacgaagacgaggatctagttgaagatgtt |
| | | gagcacttactttcagaaatgactcctgcggaggtcgcttgtttaagagagattgaaacacaactgtcacgccccgaagccgttga |
| | | ctcaaaactgaacacagtgaaatggttcttaacggaattccgtaccgatggaaaaacttggctggaacacggctgtattattttca |
| | | gccagtattacgacacggcggagtggatagcgaaagaactggccaagtccttaaaaggcgaagtggtagccgtttatgctggcgtt |
| | | ggtaaaagcggcttattcaggggcgaacagtttaataacgttgaacgcgaattgattaaatccgcagtgaagacgcgcgagattct |
| | | attagtggttgctacggatgccgcctgtgaaggcttaaacctgcaaaccttgggaacactcatcaatgtcgaccttccctggaacc |
| | | catctcgtttagagcagcgcctcgggcgaatcaaacgttttggtcagacacgtaagtttgtggatatgctcaatcttgtgtacagc |
| | | gaaacacaagacgagaaagtttataacgtgctgtcggaacgcttacgcgatacatacgacattttcggcagccttcccgatacgat |
| | | tgatgatgaatggatcgacaacgaggaagaactcaacactcgcatggatgaatacatgcatgaacgaaagaaagctcaagatgcgt |
| | | tctccgttaagtatcgcggtactctcgatcctgatgctcatctctgggaacgttgcgctacagtactgtcacgtagggacattgta |
| | | agtaagctcagcgaaccatggggaagctaattatgttgtgatgtggatgccccgctcagccaaggtcctgcacaactatgttggat |
| | | gctcttttttagagggctacatcatgaattcgatcaaagttattggtacaattctgagtaaatctgtctctcagggtatccatttc |
| | | gagtg (SEQ ID NO: 73) |
| 77 | pLG079 | gccagtcgcttgcaaagtattgagaattgatgtttatttgtgttttgaggtggtctttgaaaccaattttcgttgtcaggtcgagt |
| | | attgggtgcagcagacgctattcaaacattccgtcccggttatccgaaggtttccggctcggtagaaggcctgaagcatgtctctg |
| | | gttttgaagacggttcgggcttttccgagaggtcggactaccgaagaattgcttgttctcgtcggtgcggctttctcaaatgacaa |
| | | gcggcttgcggctctcagcgaactggagacgctatttcgcgatggtttgatagtgaaaggcaaggacggtcgctggcgtgcaaagg |
| | | cagatggtttcaaacccagacatgagagcgtgtcggcttcgagaggtggagggcctgagggcttcgttgatgtcattcacgctgcc |
| | | aatgcattcttctcctcggaaccgacggcggccgaactacctgatcaagaagacgaaagttcagatgctcccgatccgcaagcgct |
| | | actgagatattggcgctcggccttgcgtgccgatccacgaggagccacgacccaggttctcgacaaacatggaatcgagtgggcct |
| | | tgatctctgggcgtggccctatcggtccagaagaagggcaaacgctgactgtttcaatcgaactcgacgcgattgatcctgccttt |
| | | cgagaggctctggtgcgaagggaaggtcacgagaacgcgcttgcagtgggttggccgatggcggtcggacgacgtggcggagttcc |
| | | tgtctttcgacccgttggcatgttagcagcagcttgggatcgtaaggatgaccgtctaatcctgacgattgatgccgatgacgttt |
| | | tggtaaaccctgattgggtcaaaagtgccgctcgtgccagcggctggaagcgcgacgacctcgctgacctttttttcgtggacgat |
| | | gggctggggctgcgggctcaggattttgtggagaaggtaaggattgccgttgccagtcagatacgtggtcgcgttgtcggcgagaa |
| | | tctcgccacacagctcgatgcctcggctcaagggatttttgacagcgccgcgatcttcctaccgactgactcttctttcaccgcgg |
| | | gggctgctcgtgacctggatgccattgcgacatggccgaaggaccgccttgagagaactgcgcttggcgcggtattcgggtttgac |
| | | cttcaagacggcacggacaaggctgctgcaatcgacgcagttccgctgaacaaggaacagttgcgcgcggttcgatccgcatgcca |
| | | agcgcctttgaccgtcgtgaccggtccgcccgggactggcaaaagccaagcgatcgtatctatggccgcgtcagtgctcgcagatg |
| | | gtggcagtgttctcgtcgcctccaagaaccatcaagcgcttgatgctgtggaggaccgtcttggctctcttgctccggacgtccca |
| | | ttcgccatccggacactgaacccgaatgacgaggcggatacgggcttcaaggacgccctcaaacaactcatcgacagcgaaaatgt |
| | | gacgcgcaacgcatctgtcgacgaattcgcattaggcgagctcaaaagcgacgcgatcgcgagaagcgaagtggttagcgtgatcg |
| | | ataagatcacggaaacggaatgcgaaatttccgatattctggaccggattcaagtccgagaggatcgcgggcgccctgacaaccaa |
| | | gactctgaagacgtggatccgagacaaagtctcttactccgctttgtctcttggtttggatcgcttttcgccaagcgtccccccaa |
| | | agtagcgccagtgacagatcattcttcgtcccgccgcggaatgaacgtcaaagagcttcattgcgcgctggcagaaaaaagatatg |
| | | aacgcgatgcgctcgggacacctgacgatccgatcgccttaggcgagaagatccgggaagcgaccgagaatcttctgcctcgcatt |
| | | ctgtccgcccggacacatctcccagaggatgagaggcgcgaaatcgcagaactctacgatgactggacattcgacgggggacgggg |
| | | acatccccctactgatctttcgcgcgtcctcatttcgcatcggcctttgtggcttgcatcgatcttgggcacgcctcgacgcatac |
| | | ctcttgatgacgggctgtttgacctcgtgatcttcgacgaggcgagccaatgcgacatcgcgacggccgttccgttgctggcgcgc |
| | | gcgaagcgggccgtcgttgttggggatgatcgacaactgtcattcatccctcaactgggtcaggcgcaggatcgcaatctcatgca |
| | | ggctcagggcctaccggtcgccagaatgggccgtttcgcccagagtcgccgttcgctattcgatttcgcatcgcgcgtgtctgttg |
| | | ccgacaacaggattactctgaggcaccagtatcgttcagcaggccccatcgtcgattacatcagcgagaacttctacggaaaccag |
| | | ttgcagacctcgtatgacccgaggcgactgaacgtgccagatggggtgcgccctggcctcgcatgggaacatgttcctgctcccgc |
| | | ggtcccgcaaatgggcaacgtcaatccgtcggaagtaagcgcgattgttaggcacctgaaaaagctgatcgttgaagacaaataca |
| | | ctggcagcatcggtgtcataacgccgtttcgcgctcaagtggccgctatcgagaacgcggtcgatgccgtcctggatgaaccgaag |
| | | cgcattgcctgcgagctcaaggttggcacagttgacggttttcagggacaggagcgggatctcatcatgttctcgccttgcgtcgg |
| | | tccacgcagcccgcagtctggcttgaccttctttcagcgagatacgcgccgtttgaacgttgcgatttcgcgggctcgggcggtcg |
| | | cgatgatcttcggcgatcttgattttgcacgttcagggcaatcaaaagcgctggccaagctcgcttcgagggcgacggaagcgcgg |
| | | acgaaacggggcgaaggtgtgttcgacagcgattgggaacgcaaagtctatcacgctctgaaggcccgaggtctggatccgcagcc |
| | | gcagcacgaaatagctgggcggaggctggacttcgcgttgtttggagcgaatgatgtaaagctcgatctcgaggtcgacggacgca |
| | | gatggcacgaaagcccagacggtcgtcgaaagacgtcagacctgtggcgcgatcatcaactgaagtccatgggatggcgggtgcgc |
| | | cggttctgggtggacgaactttcaagggatatggagggttgtcttgaccgagtcgaacaagacctatcgtaagtcgagcaggaaca |
| | | ccgcggttgcgttggggctgggtggcgccgccatccttgcctcgggctttctcgtcctgcaagtcaactcgctcgatcgccgatat |
| | | ggtcgtatcgaggaaaatctgagctactacaccggggaactccaatccgcgcagcagcaactggcttttgctcgtgagcagtttcg |
| | | cgaactttctgaccaaaagcaaagcttgtctcaggaagtcgcgagcgccgaacgcagccttcaaagcgcggctcagagagaggcgg |
| | | atgcgcaggctagtgtcgaagcaagccaggccaaattgactgctgagcgggaccgtttggccgaagcccaaaaaacgattgcggat |
| | | gcgcagcgaattgaacgtgaaactgctcaagctttgctgcgaagaaatggcctcgaaacagaggtggtcaaactgaaaggcgatgt |
| | | gcaggcccttaaggagagccagcaagagttgtctgctggtgttgaccaaacgcaatcggctgtcgatcgcctcgaagagagaagag |
| | | ctgaacttcaacgtgaagtggatagactcgcgcccgccgttgaagaccttcgtgcacaggagcggcttgtcgaacaactgcgaggt |
| | | gacgaggatcgtctcgaacagagcctcgacgatttgaatgcgaacattgcaattgcacggactgaattggcgaccagcgcggaaaa |
| | | ggtcgatgcggccgaggagaggctgcgtgcagggcaggaacaaatagcatccacagaagctcaacttgaaacactgaatttcgaag |
| | | tcgatgacctcgagtcgagacagggcgaactgcaggcaagtgtctcgggagcagagacgcgtctttcttcattgcaaaatgaactg |
| | | gagatcgcacagaacgcggtgacgcgagctgatgcgcagcgcgctgaaactacagaagcactcaacatcgctcaggaacagttttc |
| | | gacgcgaagcgctcagctctctaccctccagtcgcagattgcatcggcagaggaagagcttgccgaacttgaagagagacgggcgg |
| | | aattcagcagattgcaggctcaaatggaccagctgcaagcacgtcgaacgacactagaggaggttctccccgatcttgagaagcga |
| | | gttcaagcagagcgggctaatttgggttctatcacgacagaagtggagacagagctcgggcgagttgctgtactcaaaggccaggg |
| | | ttccagtctggaggccgacatcgagcgcctccaagagcgtcgcgacgaactcgggctggaaacgcagtccgccactgctgaggcgg |
| | | aggccgcgcgcgcatcccttcaagctgagcttggtcaacttgcggaaaccgatgccctttcaagagcgcggactgccgatttgagg |
| | | cgcttgagagaagctcttggagctgctgaaagagagctttccgaacttgaagagagacgggcggaattcagcagattgcaggctca |
| | | aatagaccagctgcaagcacgtcgaacgacactagaggaggttctccccgaacttgagaagcgagttcaagcagagcgggctaatt |
| | | tgggttctatcacgacagaagtggaaacagagctcgggcgagttgctgaactcaaaggccagggttccagtctggaagccgacatc |
| | | gagcgcctccaagagcgtcgcgacgaactcgggctggaaacgcagtccgccactgctgaggcggaggccgcgcgcgcatcccttca |
| | | agctgagcttggtcaacttgcggaaaccgatgccctttcaagagcgcggactgccgatttgaggcgcttgagagaagctcttgctg |
| | | ctgccgatgatgagctttccgagacacgagcggaactgatggacggacagtctgtggaacaggaaccagtatcaaccattagtgaa |
| | | ggcgctggcgcccgtgaaaacgctcagtctgacaactccgcgccatcgagcaccgacaattgaggtaaccgaaaatgcttacggac |
| | | aatacaatacttgtgctggcgattgcgggtgtcctgatactgctcgccgtggttcaactttttctggccgcccgccacgaccgggc |
| | | ggttacggcagcaggcccgatcgaagagcttgccgtctacgagaagcggctggaagaaaaacagcggctcatggacgatcttgaag |
| | | ctgaagtggaaaaacgtcgggaggcaatggccgtcgttactgacctccgggctgaggtcgacggtctacggcgtcagaaggaggag |
| | | ctccttacagaatgggagagtctccgtgaacgtcgcgacgaagttgcggcagttcgcaaggagactgaggacgccgttgtcgaacg |
| | | ccagcaactcgaaacggagatcgccccgcttcgtgcggagtatctggagataaaggaaaggctggaaaaggcggaggagctcattg |
| | | agcgcactgacgccttgagacgagagcacgacgaaatctccacacaggtcaaagatcttcgggacaagaagaggcaacttgaagag |
| | | gccgaggaacgggtttctcgcctggaagagcgttccttcgaacttgagacatcgaatgctcggcttgagggacagaagtcttcgca |
| | | tgaaagcgagttgtccgccttggaagcgcggatcgcctcggaacacggtgggttggcatctgcccaaaccgaacatgctcgcctcg |
| | | atgcagaggttgcggctctgaaccaggaaacccgccgctccaggggcgaaatcgagacgctccaggacactcgaagcgcgcttgat |
| | | gctcgattggcacacctcaaggccgagatagctcgccgagaaggtcgaaccgtcgacggggaaaccggcgaaacggatccgcttcg |
| | | cgagctcaatgaaacaccaccggtcattacggagatgaggacctgggacaacgcgccccgcgagaacgaggcggatgccatcaaac |
| | | gcgtcgaacgccgcctacgcgcaaagggtctcgactacccggctcgcacgcttcgcgcttttcacaccgccatgaaagtaaatgaa |
| | | acaacgcagatggcggtccttgccggtatttccggaacgggcaagagccagctcccgcgtcaatacgcggccggtatgggcatcgg |
| | | tttcttgcaagttccggtgcagccacgttgggatagtcctcaggatctgatgggattttacaactacatcgaaggcaagttccgac |
| | | ccacagacatggcgcgtgcgctttgggcggtcgacgggcttaacaacgacgatgcggaacaggatcgcatgatgatgatcctgctg |
| | | gacgagatgaacctcgcaagggtcgaatactatttctcggacttcctcagcaggctggaaagccgtccgcgtcccgatgacgtcga |
| | | caatgaaaacgaacgcaaggacgctgtgatcgagcttgaaatcccgaacatggaacgcccccccaggatttttccgggctacaacc |
| | | tcttgtttgcgggcactatgaacgaggacgaaagcacgcagtcgctatccgataaagttgtcgaccgtgcgaatatccttcgtttt |
| | | tccgccccgaagaaaatcaaggacggacaggcagaaggaacggtcgagccgattttggccctttcgcaacagacatgggagagctg |
| | | ggggcggtcgagtgcgtctgtcgatggcggtcggcgtgtcaccaaccggattgaacaaatggttgatctgatgcgtgacttcaaac |
| | | ggcctttcggtcatcggctcggacgcgcgatcatggcttacgcggcgaactatcctgaggttgaaggcggccgcggtgtcgacgac |
| | | gctctcgcggatcaattagagatgcgccttctaccgaaactcaggggcgtggaaaccgacatggctggccctcagttctcgaggtt |
| | | gatgacctttgtggaacgcgagctgggggacgacgccttggcccaagcaatcggtgagtcaatgtccctcgccgaggcaaccgggc |
| | | agttcgtatggagtggagtcacgcgttgatgcggtttctggcccgtccctgggcggcgaaagcccttggagaggacgaagcctttg |
| | | ggcccgaagactgtctgatcggtagctaccagggggcgaacccaggcggctacgaatacgtgacgctcttgaggggaaacgtccga |
| | | ggtagcgataccggaactgttctgtttccctatccaaagcgtgaggaagctgtcgggcccgcgcgtaagggcttcccggtgcgccc |
| | | aaggtcggggcacgatcctgccactccggacgaagaagaaggcgcagaggcccttcgacacatgaacgaagttcttgcacgtatcc |
| | | aagaactggaaggtgcgattgaagacccaagcgatacatgggggcgcctgagggatgcttggaagcgcgccgaaaatgaagccgaa |
| | | cccaaaatggctgaaatcgtccggcaggcgcggggcatgcttccggtgcttcgcgatctggaaaaacgcatccgccgggttctacg |
| | | taggcacagggagctaactccccttgatcgggtgcaggagatggatcggacctctatggtgtggctcagccgacagccagggcgaa |
| | | gcatcgcggaacgtgcaggttcttcgcaacgaattcttgcgacggttcgccgtgagaatttcgatacgctcgagaaccgtgtcctg |
| | | catgcctacacgcgtcttgccgcagatgttgcacgcgaatggacccgtgagcaccctcgtgcgaaggacagtgttcgctacaaaca |
| | | ggttgaggcttttaggaaggcctgtcgagtattgtcgcgaacactcagtgacctcggtgtcatgatcgcgtcggccggcgtccagc |
| | | caaactatgtgctcatgcaagatcgcagctatcgagaggttcatgagggatggctgaggcttctcttacgccgaaaaattgtagat |
| | | gatctttgggcttggcaggccgaaacttggacggatttctccgttctttcgatcattcttgccatcgacgaattggaagaggctga |
| | | acttgtcgctcagtcgccgatttcgtggagcggtgaggcaacaggcggacgctggttcaatcaggatcggccaatcgccgtctttt |
| | | ggctgcgcgacaccaaccgcattgttgaagtccaagcacgccctgagcgaccaggaaccatgttgagcgcggcacaagcgcacgtc |
| | | gccctcagaatttccgatcccaaacgggctgaccttccgcgcaggatcgctgtctggacgccacatgccatgcgtagaattgatct |
| | | cgaggatactgtgcggggggcagttcaactgcttcaccaaatccagcccctcgctcagacggaagttttgcggaatgggttgatca |
| | | tgaccccagcacgtggtgtcgcagctgaagagagcgcaactcacggaagagcgatcgttacggcaatcgccataggcccagccggt |
| | | gaagacctagcgaagggattccaggccgtgcgcgacttcattcgcagtgagctatacgaggtcgcaacatgatcgaccgaaaacta |
| | | tgcggcttcgatctcaacggatggagagatttcgttgcgaagaactggcgctccgtgccaggtgaagacgaggtcattggtccgac |
| | | cgatatcgtcacaagtggccctctttcgtcgatcgtgcggatcggggaaagccgcctcgcaggttggatcggaggaccgcaggctg |
| | | acattgctccgcacggtcgcggtggtggttggggtgatgtcgggtcagaacaaagacgcattcccgttcggtcactgctggaaatg |
| | | cgtgatgacggggtcgaaaaactcgcccaggcacttgtgggatctgcgagcggttcggcaaacacagtcgtttcgatcgatgaggg |
| | | cccggatggcgatgaagccgtccaagagcaccttctcgaagcacttgcccgagggaagttccgaaatggctcattggtttggcgac |
| | | cagttcttgccgccttgttcgccattcatcgcgatcaggtttcggaggggcagcttgtaggcgtcgtctcccatcagcgccaaggc |
| | | ttgtcagttcaaaagctgcgtattcgtagcgcaaggaatgtgctcgccccggagcgacgcgaggccgctgcccatataccgtgcga |
| | | cgctggttacgagtccctattccgaggtgcccgcaacgccgctgtcggggcagagggtttttcggcgcgcacagctcatcgtgcga |
| | | tcgcaagctcggtcggaaaagctggtttagggatggattgcaatcctgagatgctccgcatgcccaacggcgattgggagctcttg |
| | | gaccttaataaatttgacgcgtcggaagtggtgagtgtcccgagttccgagctcgatctggccgattgcgacgtcgttcttttcga |
| | | gaccctttgtgaaggtcggctcaaaaaatgcctgagtgatgctatccaaagagcagctccagtcgaggtgctctctcttcccgcaa |
| | | cggctgttgcggaaggtgccttggaagcagcacgccgagccggggacggggaaccgatcttcttcgactttctaccacgattgtcc |
| | | accatcgtgttcggatcggatggcgcaaagaatttcgatctcatacggaaagaagaaacgctcgaagcaggccggacctacagaag |
| | | ccctgaagcagcatctctcgcgataccggcagggcaggagagcgtctctgtctacctgaggaaagaggaagctccctggcctcgaa |
| | | aggcaagggtgtcgcttggagctcctctgaagcatcaagctgccgtctcgctgtgggtcgaacagaaaccggccgccgggcgagcg |
| | | cggatcctcatggaatcgccggacttggggcggaatttcgcggtggattgggatgaagcactggaagaggaacggccctggtctga |
| | | gatcatcgagagcttggatacgcaagtgtcaattcccaaacgtctggttcttccctgcggcatggaggcatggcatgacagcgatc |
| | | gatccgcaggtatgctaactttgctcgaatccgagcctaatcgcagccgcacggattgggcgacccttcggcaaaaactttcacag |
| | | cgtccctttggcaaatactgcatctcaagtgacggcgacgtgcctccggagatcgcggcagaaaccctcgagcggtttgaaattct |
| | | gaccagcaaagcgcttgaggttactgaaaagcgcctgaggggcgaaagcggctacggaacggaagacaatgaggctctcaaattct |
| | | tgagttggcagttccgccgatgcccgcgcgatgtcgcgacgtggctgatggactgtattgaagcgtccgggcgcaaccatccgttc |
| | | gtcaaacatcaagcaagttgggttctcgtatatcagggccttggccgcatcgtcggaaacgaagaggacgaagcgagagcaatgcg |
| | | gttgcttctgacttcgtccattgaggactgggtctggaaccgacaaagcgcggccatggcgttcatgctgtctcgttctgacagcg |
| | | ctccatcttacctggaacgagaagacgtagagaagctgaccaagaggactatcgcggacttccaacgtaatatcggcggccaatat |
| | | acaatgtttaactacgcgcctttcttacttgcaggcctgataagatggcgtctcgttgatcctaaagctttggtgatcggggccga |
| | | cccgttggcggatgacctcttggctatcattgagaaaacagagcacgacctgaaggcccgttgtgggtccaatatgaatttccaaa |
| | | ggcggcggtcgaagttcttgcctatcctccaagacctgaagtcagagctggcgggagaaggttcgaatcctgacctgttgttggat |
| | | atctatggagcgagcggaacgtgaccatgagcgcgcaggtaccaagctgctggatcaagcctctgggacgagcagaagcccgtccg |
| | | ttgtataaaatcaccacagacacaagcaagagatatcgtgatactaaagcgctctggaggattcccatcagacctgatgaacatcg |
| | | caactgcatcgaaccagaaccatcttggcaactaggtgcggaccaaggactgaagcatgtgcctatcgctcaa |
| | | (SEQ ID NO: 74) |
| 78 | pLG080 | gggctgtttggttgaattaaaaatacgaactaaaaccaacaagagtcggaaaaaacttcaaaatgctgcttatggataatagtcatc |
| | | ttaaaaatgtacggaaaaagagactaaaatcagaaaaacatctgttatacattgacttaaagtcatcatctccgctatgagtcctca |
| | | atccaagttgacaaatgtttagccaggagttcccgtgaacgagcatctctctcatatggatgtacataccttgtttgaagaaatgga |
| | | cgagcaggctgatggaataacgtttaaatactcatttgatgacatagcaaagagcaacgcattggttgtcactgagtttgtcaattt |
| | | tgagcgtgacagcacggtagctttactcgccagccttcttactctcccggcacaccaatctcagtgtttgcgctttgagcttctgac |
| | | gagccttgcactaattcactgcaaaggtcagcagatagcaaatatcgatgacgtgaaacgctggtatgtcactattggggagtcgag |
| | | tagtatcgttggagaagatcctgctgaggacgtcttcgtcgcccttgttgataataaaaaaggtgattaccgtgtgctagagggggt |
| | | ttgggaggcggcaggtttttatacacaattaatggtcgaaattgtatccgacatgccggatacgcaccgctatcgctcgctgaaact |
| | | tgctatacaggcaattctccgtctctcagatgtcatttgtgctcgctctggcctttatcgttttcaggaaggcgcagacgaattccc |
| | | tgactctcttgacaccgctggtcttgatgagaaaacgctctgttcaagggtaacgttgtccgagcgttctcttcgagctgaggggat |
| | | caaacttgctgacttagcacctttcattcttgaaccttctcatataagtatgcttggaaatcaggtccctggggagggaatgcttga |
| | | acaacggccattgctccgcacacgcgatggtattgtggttgtacttcctaccgccatgaccattgcacttcgccaggcagtgataac |
| | | atttgcaaagcgcacagaagaattgagcgagctagacaaagcgttagctaacgtctacagccttactttctccgagatgccggtctt |
| | | cggtaatggaggaaggttaagaagactgacatgggagaagtacaaaatgagccgaacaacgatggtaacctccatcgtggatgctgg |
| | | tcatttgatggtacttcagttcgttttgccttccatacagcaatatgccgataccggtttcaacaacttgctacagctagatgaaga |
| | | gaccacgcaatttctagataactctgttgaacaaattacagttgacctcgccaaacaacccggctttcagcgtggcatcgtcgtgcg |
| | | cattgcatgtgggtggggggcgggttttatgggggtccctccccaactgccagatggttggggatttgaatggatgtctggtgcgga |
| | | ctttgtccggttcggggcattacccgatatgtcaccaattgccttctggcgtgtgcaagacgcagtcgaaacgatcaggcaagctgg |
| | | tgttcgattaatcaatatgagcggaactctcaatcttcttgggtggatacgtgccaatgatggccatatggttcctcatgaccagtt |
| | | accagatgaccgtatcacaccggaacacccgctaatgttaatgattcccacgaatttactccgtggtatacgaatagcggcagacac |
| | | aggatatgaccggcatcgcattagtgacaacaatggtaaatggcatcgagtgatgaggccttcggcagaagatttctttcccaccga |
| | | gcgtcagagcaagtgctacgcatcaattgatgatcttgaagcgcaacggctgacctgtgtatatgaggggcagggtaatctttggg |
| | | taacgctcgaagctccagaaatggaagattggatgctcctcgttgagcttgccaaaatggttcgaacatggattgggcggattggc |
| | | gaggcactggaggtcttgagtgagcaaccaataaaaaaatcattaaaggtgtatctgcattttgatggtaacgacaatatcggcag |
| | | atttgatggtgagaatttttctgatgatatgaatacattttggcgacttgaacgaatccatgagcatggggcgattcgtgtggttc |
| | | ttcaagatgggtatcttgcaggttttcgtctaccggataaccgtgcagaacgagctctggtgcgcgcactcggtacggcgtttgcc |
| | | acacttcttcggatgaaagagccagtagacaaaggggtcactgttgagcagatagcggtgcccaatgacagagcgcgcagcttcca |
| | | cataatgcaggcttatgacttcaaccaatatttaggccgttcactaactaaacgtcttttagctattgaagatatcgactcagccg |
| | | cagcccgaattgagctagcatggcgtgctgtttcgacagatgcaccatcacgatatcagggtaaaaaggaagttggaaagctcctt |
| | | aatgatgtggttgatgtgctgatccaagacttactaagcgaactttcaagatttgaccgtaaacagacagtaatgcgattacttga |
| | | aaacgttgtaaaggcacgttgtgaagaggcgcactggcgtagtactgcagcagcggtccttggcttgcatgcaggagaagagggtg |
| | | tcgaagagacgatagctcaagaaatgagccgttatgcgggcgcagcgttaacttcccggctaatcattgaacttgccatctgtgtg |
| | | tgcccgacaagcggtggaattgaaccttctgatatggcactcagtaaacttcttgcacgggcatcactgctttttcgcataggtgg |
| | | tatgtcagatgccgtacgtttcggtgctttgcctgctgatattcgcatctcccccttaggtgatctcctctttcgcgatgaactcg |
| | | gcaaaatggtgcttgaaccaatgctttcaaaagttactaacgaacggtttgaggaacaagcggcacaattcgagcaacactatgtg |
| | | aaaactgccggaggggatgatgagaatagcaaacaagatagtgttgcggctgaaaccaccgaggaccaaaccgatattttccttgc |
| | | attctggaaagcagaaatgggcttcactctcgaggatggaatgcgatttatccagttccttgagtccatcggaatagagcaagaat |
| | | cagcaatcttcgagatgcgaagaagccaattagcggatgctgctaaatcggctgggctcgcagatgaaactattgatgcgttcctc |
| | | aaccagtttatccttagcgcgcgtccgaaatgggatgtagtgcccgatggatttgacctttctgatatatatccctggaggtttgg |
| | | ccgacgcctttcagttgctgtacgtcccttgttacagattgaagagagtcacgatccactaattgttatcgcaccaggactcttga |
| | | atctgtcccttaaatacgttttcgatggcgcatacactgggcaatttaagcgtgacttctttcgcacagagggtatgagagacact |
| | | tggttaggtggagcgcgggaaggacacacattcgaaaaaactttggagagagaacttcgtgaaataggctggacagttcgacgtgg |
| | | cataggctttcctgaaattcttcgcaggaatctaccaggtgatccgggggatattgatcttcttgcctggcgctcagaccgcaatc |
| | | aagttctcgttatcgaatgtaaggacctctcacttgctcgtaattactcagaagttgcctcgcaactatctgaatatcaaggtgat |
| | | gacataaagggcaaaccagataaactcaagaaacaccttaaacgcgtattactagccaaagaaaacatcgataattttgccaagtt |
| | | cacttcgatagcgaatcccgagattgtatcgtggctcgttttcagtggagcatctcccattgcctatgctcaatccaagattgagg |
| | | ctttggcaggaactaatgttggccgcccaagtgatcttctgaacttttgatagatatgctgtgcgataagacgccctggcaactaa |
| | | gttaatcgttcctactactgatagttttaaatcaagg (SEQ ID NO: 75) |

### Variants and Mutations

One or more components of the systems herein may comprise one or more mutations compared to corresponding wildtype counterparts. In some embodiments, the one or more mutations may be in the catalytic domain of an enzyme of a system herein. The mutation(s) may alter (e.g., increase) the activity of the enzyme.

### Polynucleotides and Vectors

The present disclosure further includes polynucleotides comprising coding sequences of one or more components of the systems. In some embodiments, the present disclosure comprise vectors. The vectors may comprise the polynucleotides with coding sequences of one or more components of the systems. In one aspect, the present disclosure provides cells comprising one or more of the polynucleotides and/or vectors herein.

A vector refers to a nucleic acid molecule capable of transporting another nucleic acid to which it has been linked. A vector may be a replicon, such as a plasmid, phage, or cosmid, into which another DNA segment may be inserted so as to bring about the replication of the inserted segment. Generally, a vector is capable of replication when associated with the proper control elements. Examples of vectors include nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques.

Certain vectors may be capable of directing the expression of genes to which they are operatively-linked. Such vectors are referred to herein as “expression vectors.” Common expression vectors of utility in recombinant DNA techniques are often in the form of plasmids. A vector may be a recombinant expression vector that comprises a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. As used herein, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

A vector may be a viral vector, wherein virally-derived DNA or RNA sequences are present in the vector for packaging into a virus. Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some embodiments, the polynucleotide herein may be a part of a vector or a pair of vectors that is/are introduced into cells for inducing diversification (e.g., site-specific mutagenesis) of the variable region and/or support replication of the molecules. Non-limiting examples of vectors include plasmids and virus based vectors, including vectors for phage display that may be used to express a diversified variable region sequence. Other non-limiting embodiments are vectors containing variable sequences that have been subjected to the methods of the instant invention and then removed from an operably linked template region, including by preventing the expression of template regions, so as to produce without further diversification quantities of the variable region-encoded protein for uses including as a diagnostic, prognostic, or therapeutic product.

### Regulatory Sequences

The vectors or polynucleotides may further comprise one or more regulatory sequences. In some cases, the regulatory sequences may direct the expression of the nucleic acids in specific types. The term “operably linked” as used herein refers to linkage of a regulatory sequence to from a DNA sequence such that the regulatory sequence regulates the mediates transcription of the DNA sequence. Regulatory sequences include transcription control sequences, e.g., sequences which control the initiation, elongation and termination of transcription. In some cases, regulatory sequences include those control transcriptions. Examples of such regulatory sequences include promoters, enhancers, operators, repressor, transcription terminator sequences.

The variable region (or the gene overlapping or including the variable region sequence), the template region, and the coding sequence for reverse transcriptase may be operably linked to the same regulatory sequence (e.g., promoter). Alternatively or additionally, the variable region (or the gene overlapping or including the variable region sequence), the template region, and the coding sequence for reverse transcriptase may be operably linked to different regulatory sequences. In some cases, the variable region (or the gene overlapping or including the variable region sequence) and the template region are operably linked to the same regulatory sequence; and the encoding sequence for reverse transcriptase is operably linked to a different regulatory sequence. In some cases, the template region and the coding sequence for reverse transcriptase are operably linked to the same regulatory sequence; and the variable region (or the gene overlapping or including the variable region sequence) is operably linked to a different regulatory sequence.

### Promoters

In some examples, the regulatory sequences are promoters. The promoter may be suitable for expressing the component(s) in the systems, e.g., the variable region, the template region, and/or the reverse transcriptase in desired cells. A promoter refers to a nucleic acid sequence that directs the transcription of a operably linked sequence into mRNA. The promoter or promoter region may provide a recognition site for RNA polymerase and the other factors necessary for proper initiation of transcription when a sequence operably linked to a promoter is controlled or driven by the promoter. A promoter may include at least the Core promoter, e.g., a sequence for initiating transcription. The promoter may further at least the Proximal promoter, e.g., a proximal sequence upstream of the gene that tends to contain primary regulatory elements. The promoter may also include the Distal promoter, e.g., the distal sequence upstream of the gene that may contain additional regulatory elements. In some cases, the promoter may be a heterologous promoter, e.g., promoting expression of nucleic acids or proteins in cells that do not normally make the nucleic acids or proteins.

The promoters may be from about 50 to about 2000 base pairs (bp), from about 100 bp to about 1000 bp, from about 50 bp to about 150 bp, from about 100 bp to about 200 bp, from about 150 bp to about 250 bp, from about 200 bp to about 300 bp, from about 250 bp to about 350 bp, from about 300 bp to about 400 bp, from about 350 bp to about 450 bp, from about 400 bp to about 500 bp, from about 450 bp to about 550 bp, from about 500 bp to about 600 bp, from about 550 bp to about 650 bp, from about 600 bp to about 700 bp, from about 650 bp to about 750 bp, from about 700 bp to about 800 bp, from about 750 bp to about 850 bp, from about 800 bp to about 900 bp, from about 850 bp to about 950 bp, from about 900 bp to about 1000 bp, from about 950 bp to about 1050 bp, from about 1000 bp to about 1100 bp in length.

The promoters may include sequences that bind to regulatory proteins. In some examples, the regulatory sequences may be sequences that bind to transcription activators. In certain examples, the regulatory sequences may be sequences that bind to transcription repressors.

In some cases, the promoter may be a constitutive promoter, e.g., U6 and H1 promoters, retroviral Rous sarcoma virus (RSV) LTR promoter, cytomegalovirus (CMV) promoter, SV40 promoter, dihydrofolate reductase promoter, β-actin promoter, phosphoglycerol kinase (PGK) promoter, ubiquitin C, U5 snRNA, U7 snRNA, tRNA promoters or EF1α promoter. In certain cases, the promoter may be a tissue-specific promoter may direct expression primarily in a desired tissue of interest, such as muscle, neuron, bone, skin, blood, specific organs (e.g. liver, pancreas), or particular cell types (e.g. lymphocytes). Examples of tissue-specific promoters include Ick, myogenin, or thy1 promoters. In some embodiments, the promoter may direct expression in a temporal-dependent manner, such as in a cell-cycle dependent or developmental stage-dependent manner, which may or may not also be tissue or cell-type specific.

In some cases, the promoters may be inducible promoters. The term “inducible promoter”, as used herein, refers to a promoter that, in the absence of an inducer (such as a chemical and/or biological agent), does not direct expression, or directs low levels of expression of an operably linked gene (including cDNA), and, in response to an inducer, its ability to direct expression is enhanced. Examples of inducible promoters include, promoters that respond to heavy metals, to thermal shocks, to hormones, promoters that respond to chemical agents, such as glucose, lactose, galactose or antibiotic (e.g., tetracycline or doxycycline). Examples of inducible promoters also include Drug-inducible promoters, for example tetracycline/doxycycline inducible promoters, tamoxifen-inducible promoters, as well as promoters that depend on a recombination event in order to be active, for example the cre-mediated recombination of loxP sites. Examples of inducible promoters further include physically-inducible promoters, e.g., particular a temperature-inducible promoter or a light-inducible promoter.

The promoters may be suitable for expressing the component(s) in the systems in desired types of cells. In some cases, the promoters are for expressing the component(s) in prokaryotic cells. Examples of such promoters include filamentous haemagglutinin promoter (fhaP), lac promoter, tac promoter, trc promoter, phoA promoter, lacUV5 promoter, and the araBAD promoter. In some cases, the promoters are for expressing the component(s) in eukaryotic cells. Examples of such promoters include the cytomegalovirus (CMV) promoter, human elongation factor-1E promoter, human ubiquitin C (UbC) promoter, and SV40 early promoter. In some examples, the promoters are for expressing the component(s) in yeasts. Examples of such promoters include Gal 11 promoter and Gal 1 promoter. In some cases, the promoters may be used for expressing the components in a cell-free system. In such cases, the promoters may be selected based upon the source of the cellular transcription components, such as RNA polymerase, that are used.

### Codon Optimization

In some embodiments, at least one or more regions of the polynucleotide molecule may be codon optimized for expression in a eukaryotic cell. In certain embodiments, the polynucleotide molecules that encode one or more components of the systems as described in any of the embodiments herein are optimized for expression in a mammalian cell or a plant cell.

An example of a codon optimized sequence is in this instance a sequence optimized for expression in a eukaryote, e.g., humans (i.e. being optimized for expression in humans), or for another eukaryote, animal or mammal as herein discussed. It will be appreciated that other examples are possible and codon optimization for a host species other than human, or for codon optimization for specific organs is known. In some embodiments, an enzyme coding sequence encoding a component in the system is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence.

Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a component in the system corresponds to the most frequently used codon for a particular amino acid.

### Nuclear Localization Signals

In some embodiments, the systems and compositions herein further comprises one or more nuclear localization signals (NLSs) capable of driving the accumulation of the components, to a desired amount in the nucleus of a cell.

In certain embodiments, at least one nuclear localization signal (NLS) is attached to the nucleic acid sequences encoding the components in the systems. In some embodiments, one or more C-terminal or N-terminal NLSs are attached (and hence nucleic acid molecule(s) coding for the components in the systems can include coding for NLS(s) so that the expressed product has the NLS(s) attached or connected). In a preferred embodiment a C-terminal NLS is attached for optimal expression and nuclear targeting in eukaryotic cells, e.g., human cells.

Non-limiting examples of NLSs include an NLS sequence derived from: the NLS of the SV40 virus large T-antigen; the NLS from nucleoplasmin (e.g., the nucleoplasmin bipartite NLS; the c-myc NLS; the hRNPA1 M9 NLS; the sequence of the IBB domain from importin-alpha; the NLSs of the myoma T protein; the NLS of human p53; the NLS of mouse c-abl IV; the NLSs of the influenza virus NS1; the NLS of the Hepatitis virus delta antigen; the NLS of the mouse Mx1 protein; the NLS of the human poly(ADP-ribose) polymerase; and the NLS of the steroid hormone receptors (human) glucocorticoid. Examples of such NLSs include those described in paragraph [00131] in Zhang et al. WO2014093595A1.

In some embodiments, a NLS is a heterologous NLS. For example, the NLS is not naturally present in the molecule it attached to.

In general, strength of nuclear localization activity may derive from the number of NLSs in the nucleic acid-targeting effector protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI).

In some embodiments, a vector described herein (e.g., those comprising polynucleotides encoding the components in the systems comprise one or more nuclear localization sequences (NLSs), such as about or more than about 1, 2, 3, 4, 5, 6, 7, 8, 9, 10, or more NLSs. More particularly, vector comprises one or more NLSs not naturally present in the the components in the systems. Most particularly, the NLS may be present in the vector 5′ and/or 3′ of the the components in the systems. In some embodiments, the the components in the systems comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus.

In certain embodiments, other localization tags may be fused to the Cas and/or transposase(s), such as without limitation for localizing to particular sites in a cell, such as organelles, such mitochondria, plastids, chloroplast, vesicles, golgi, (nuclear or cellular) membranes, ribosomes, nucleoluse, ER, cytoskeleton, vacuoles, centrosome, nucleosome, granules, centrioles, etc.

### Fusion Proteins and Linkers

The components, e.g., proteins, domains, and nucleic acids, in the systems (from the same or different systems) may be associated (e.g., fused). The fusion may be via a linker. The term “linker” as used in reference to a fusion protein refers to a molecule which joins the proteins to form a fusion protein. Generally, such molecules have no specific biological activity other than to join or to preserve some minimum distance or other spatial relationship between the proteins. However, in certain embodiments, the linker may be selected to influence some property of the linker and/or the fusion protein such as the folding, net charge, or hydrophobicity of the linker. In some embodiments, components in different systems may be associated (e.g., fused). In some embodiments, the two or more different systems herein may be associated (e.g., fused). For example, two or more of the ATPase(s), deaminase(s), and reverse transcriptase(s) may be associated (e.g., fused) together.

Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the Cas protein and the ligase by a distance sufficient to ensure that each protein retains its required functional property. Preferred peptide linker sequences adopt a flexible extended conformation and do not exhibit a propensity for developing an ordered secondary structure. In certain embodiments, the linker can be a chemical moiety which can be monomeric, dimeric, multimeric or polymeric. Preferably, the linker comprises amino acids. Typical amino acids in flexible linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180. For example, GlySer linkers GGS, GGGS (SEQ ID NO: 76) or GSG can be used. GGS, GSG, GGGS (SEQ ID NO: 76) or GGGGS (SEQ ID NO: 77) linkers can be used in repeats of 3 (such as (GGS)₃ (SEQ ID NO: 78), (GGGGS)₃ (SEQ ID NO: 79)) or 5, 6, 7, 9 or even 12 or more, to provide suitable lengths. In some cases, the linker may be (GGGGS)₃₋₁₅, For example, in some cases, the linker may be (GGGGS)₃₋₁₁, e.g., GGGGS (SEQ ID NO: 77), (GGGGS)₂ (SEQ ID NO: 80), (GGGGS)₃ (SEQ ID NO: 79), (GGGGS)₄ (SEQ ID NO: 81), (GGGGS)₅ (SEQ ID NO: 82), (GGGGS)₆ (SEQ ID NO: 83), (GGGGS)₇ (SEQ ID NO: 84), (GGGGS)₈ (SEQ ID NO: 85), (GGGGS)₉ (SEQ ID NO: 86), (GGGGS)₁₀ (SEQ ID NO: 87), or (GGGGS)₁₁ (SEQ ID NO: 88).

In particular embodiments, linkers such as (GGGGS)₃ (SEQ ID NO: 79) are preferably used herein. (GGGGS)₆ (SEQ ID NO: 83), (GGGGS)₉ (SEQ ID NO: 86) or (GGGGS)₁₂ (SEQ ID NO: 89) may preferably be used as alternatives. Other preferred alternatives are (GGGGS)₁ (SEQ ID NO: 77), (GGGGS)₂ (SEQ ID NO: 80), (GGGGS)₄ (SEQ ID NO: 81), (GGGGS)₅ (SEQ ID NO: 82), (GGGGS)₇ (SEQ ID NO: 84), (GGGGS)₈ (SEQ ID NO: 85), (GGGGS)₁₀ (SEQ ID NO: 87), or (GGGGS)₁₁ (SEQ ID NO: 88). In yet a further embodiment, LEPGEKPYKCPECGKSFSQSGALTRHQRTHTR (SEQ ID NO: 90) is used as a linker. In yet an additional embodiment, the linker is an XTEN linker. In particular embodiments, the CRISPR-cas protein is a Cas protein and is linked to the ligase or its catalytic domain by means of an LEPGEKPYKCPECGKSFSQSGALTRHQRTHTR (SEQ ID NO: 90) linker. In further particular embodiments, the Cas protein is linked C-terminally to the N-terminus of a ligase or its catalytic domain by means of an LEPGEKPYKCPECGKSFSQSGALTRHQRTHTR (SEQ ID NO: 90) linker. In addition, N- and C-terminal NLSs can also function as linker (e.g., PKKKRKVEASSPKKRKVEAS (SEQ ID NO: 91)).

Examples of linkers are shown in the Table 4 below.

TABLE 4

| | |
|---|---|
| GGS | GGTGGTAGT (SEQ ID NO: 92) |
| GGSx3 (9) | GGTGGTAGTGGAGGGAGCGGCGGTTCA |
| | (SEQ ID NO: 93) |
| GGSx7 (21) | ggtggaggaggctctggtggaggcggtagcggaggcgg |
| | agggtcgGGTGGTAGTGGAGGGAGCGGCGGTTCA |
| | (SEQ ID NO: 94) |
| XTEN | TCGGGATCTGAGACGCCTGGGACCTCGGAATCGGCTAC |
| | GCCCGAAAGT (SEQ ID NO: 95) |
| Z-EGFR_ | Gtggataacaaatttaacaaagaaatgtgggcggcgtgg |
| Short | gaagaaattcgtaacctgccgaacctgaacggctggcag |
| | atgaccgcgtttattgcgagcctggtggatgatccgagc |
| | cagagcgcgaacctgctggcggaagcgaaaaaactgaac |
| | gatgcgcaggcgccgaaaaccggcggtggttctggt |
| | (SEQ ID NO: 96) |
| GSAT | Ggtggttctgccggtggctccggttctggctccagcggt |
| | ggcagctctggtgcgtccggcacgggtactgcgggtggc |
| | actggcagcggttccggtactggctctggc |
| | (SEQ ID NO: 97) |

### Adaptor Proteins

The adaptor proteins may include orthogonal RNA-binding protein/aptamer combinations that exist within the diversity of bacteriophage coat proteins. A list of such coat proteins includes, but is not limited to: Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1.

### Heterologous Components

In some embodiments, when a system or composition herein comprises multiple components, the components may be heterologous, i.e., they do not naturally occur together in the same cell or an organism. In some examples, the system comprises an ATPase and an adenosine deaminase that are heterologous. In certain examples, the system comprises two or more heterologous reverse transcriptases.

### Cas Proteins and Variants

In some embodiments, the systems may further comprise a Cas protein or a variant thereof, and one or more guide molecules. One or more components described herein in the systems may be associated (e.g., fused) with a Cas protein or a variant thereof (a catalytically inactive). The Cas protein and guide molecule(s) may guide the components such as ATPase, deaminase, reverse transcriptase etc. to target a desired target sequence.

The Cas proteins, variants thereof, and guide molecules may be those in a CRISPR-Cas or CRISPR system, refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molce1.2015.10.008.

### Class 1 Systems

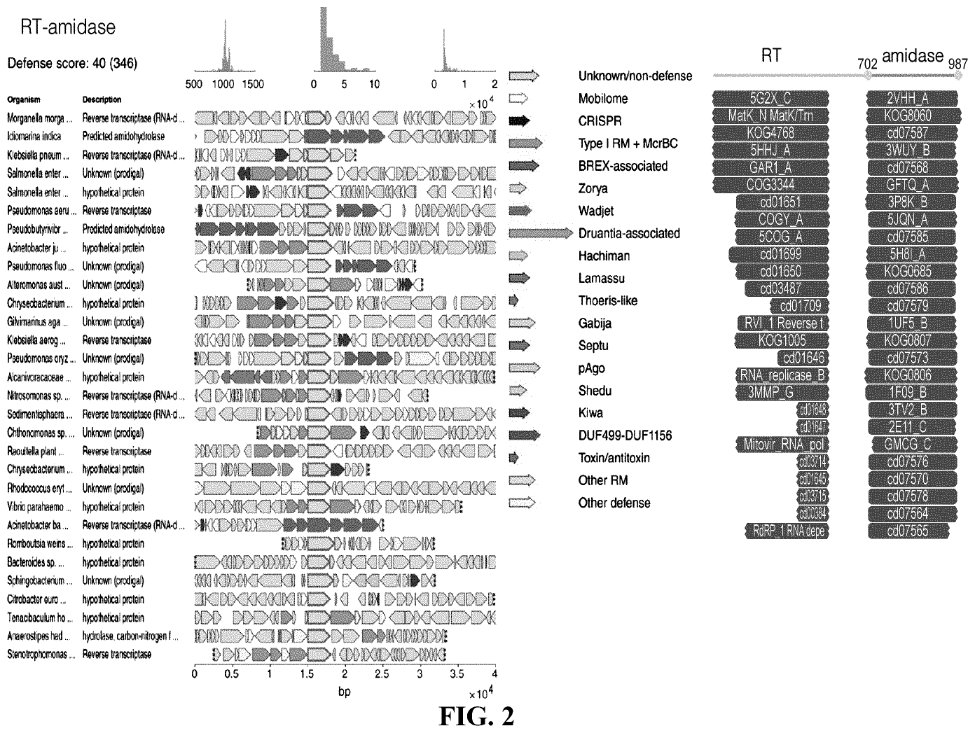

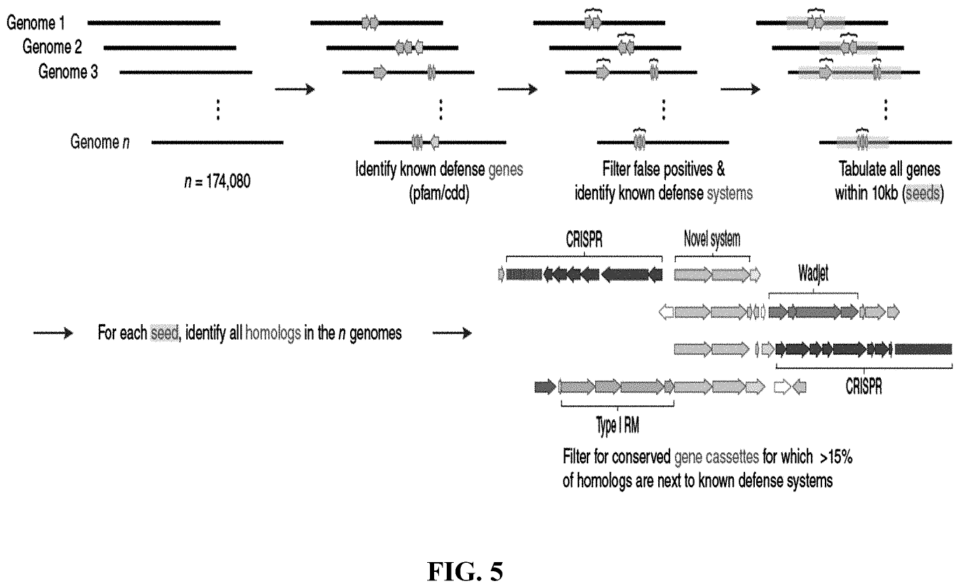

The Cas proteins may be Cas proteins in class 1 CRISPR systems. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g. Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas⁷ subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

The Cas proteins may be Cas proteins in class 2 CRISPR-Cas systems. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SETT/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### Guide Molecules

The guide molecules (i.e., a molecule comprising a guide sequence) refer to polynucleotides capable of guiding Cas to a target genomic locus and are used interchangeably as in foregoing cited documents such as International Patent Publication No. WO 2014/093622 (PCT/US2013/074667). In general, a guide molecule may be any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, e.g., a single stem loop. In certain embodiments, the direct repeat sequence forms a stem loop, e.g., a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Methods of Identifying Defense Systems

The present disclosure further provides methods of identifying defense systems. In some embodiments, the methods are based on the facts that genes of defense systems often form clusters in the genome. Thus, candidate defense system genes may be those co-locate with known defense system genes in the genomes of multiple cells of a species or strain. Accordingly, novel defense system be identified by recording or identifying candidate genes located close to known defense systems and identifying homologs of the candidate genes in multiple genomes of the species or cells. The candidate genes that have a significant number of homologs close to known defense system genes may be selected as a putative novel defense system genes. The selected putative defense system genes may be further validated by experiments, e.g., by testing their effects on phage resistance.

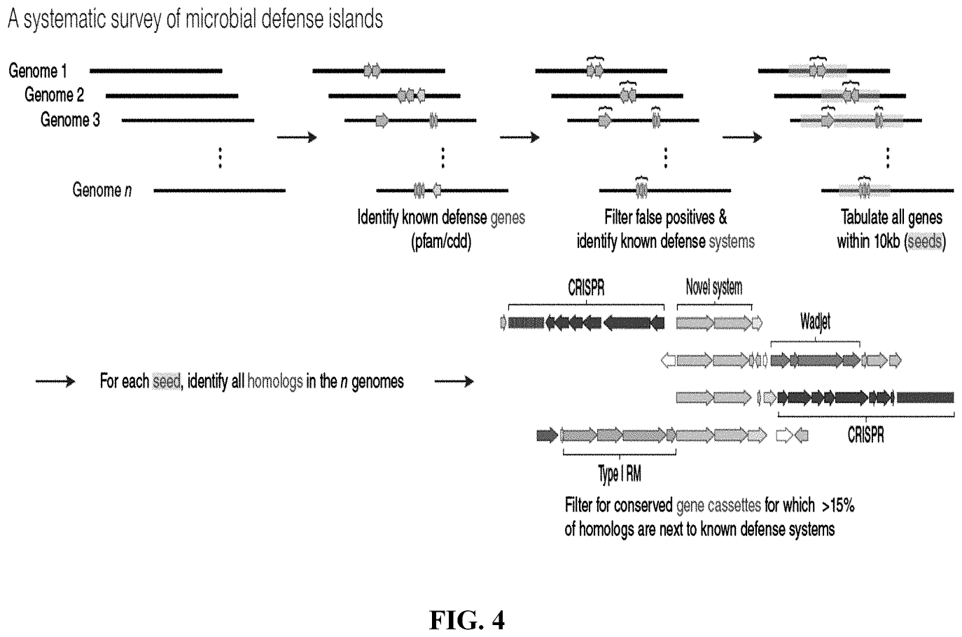

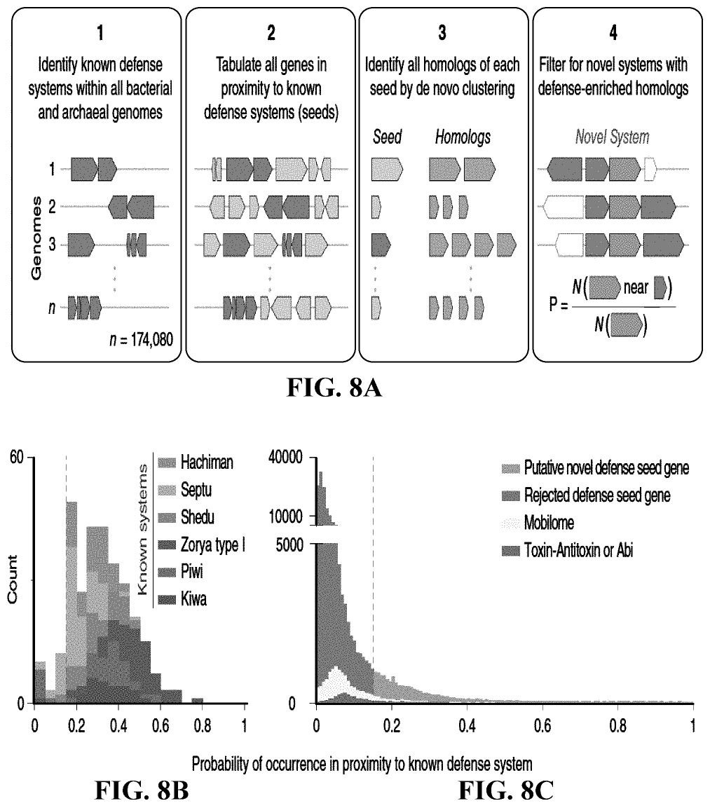

In some examples, the methods of identifying a defense system in a microorganism may comprise identifying genes of known defense systems in a plurality of genomes of the microorganism; recording candidate genes located within 50 kb from the identified genes of known defense systems on the genomes; identifying homologs of each candidate gene on the genomes; and selecting candidate genes wherein at least 10% of homologs of the candidate genes are within 5000 nucleotides and/or 5 genes from one or more known defense systems on the genomes. FIGS. 4 and 8 show flow charts of exemplary methods of identifying novel defense systems.

In some cases, the recorded candidate genes may be located less than 50 kb, less than 40 kb, less than 30 kb, less than 20 kb, less than 10 kb, less than 8 kb, less than 6 kb, less than 4 kb, less than 2 kb, less than 1000 bp, less than 800 bp, less than 600 bp, less than 400 bp, or less than 200 bp from the identified genes of known defense systems on the genomes. In some cases, the recorded candidate genes may be located less than 20, less than 18, less than 16, less than 14, less than 12, less than 10, less than 8, less than 6, less than 4, or less than 2 open reading frames from the identified genes of known defense systems on the genomes.

The methods of identifying defense systems may comprise obtaining sequence data of multiple genomes. The multiple genomes may be those from different microorganism cells of the same species or strain. The sequence data used may be from at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 400, at least 600, at least 800, at least 1000, at least 2000, at least 4000, at least 8000, at least 10,000, at least 20,000, at least 40,000, at least 60,000, at least 80,000, at least 100,000, at least 120,000, at least 140,000, at least 160,000, at least 180,000, or at least 200,000 genomes.

The methods of identifying defense systems may comprise identifying known defense system genes in multiple genomes. The known defense systems or their genes may be identified using sequence alignments and comparing with known sequences, motifs or domains in a protein or nucleic acid domain database. The domains within the gene members of each system may be analyzed bioinformatically using the tools HHpred (Soding J, Biegert A, Lupas A N. (2005) The HHpred interactive server for protein homology detection and structure prediction, nucleic Acids Res. 33: W244-W248; Alva V, Nam S-Z, Soding J, Lupas A N, I. S, S. C, et al. (2016) The MPI bioinformatics Toolkit as an integrative platform for advanced protein sequence and structure analysis, nucleic Acids Res. Oxford University Press; 44: W410-W415), Phyre2 (Kelley L A, Mezulis S, Yates C M, Wass M N, Sternberg M J E. (2015) The Phyre2 web portal for protein modeling, prediction and analysis. Nat Protoc. Nature Research; 10: 845-858), PSI-BLAST (Altschul S F, Madden T L, Schaffer A A, Zhang J, Zhang Z, Miller W, et al. (1997) Gapped BLAST and PSI-BLAST: a new generation of protein database search programs, nucleic Acids Res. 25: 3389-402).

In some examples, the database may be PFAM. The term “pfam” may encompass a large collection of protein domains and protein families maintained by the pfam consortium and available at several sponsored world wide web sites, including for example: pfam.sanger.ac.uk/(Welcome Trust, Sanger Institute); pfam.sbc.su.se/ (Stockholm Bioinformatics Center); pfam(dot)janelia(dot)org/(Janelia Farm, Howard Hughes Medical Institute); pfam(dot)jouy(dot)inra(dot)fr/(Institut national de la Recherche Agronomique); and pfam.ccbb.re.kr/. pfam domains and families are identified using multiple sequence alignments and hidden Markov models (HMMs) (see e.g. R. D. Finnet et al. nucleic Acids Research Database (2010) Issue 38: D211-222). By accessing the pfam database, for example, using any of the above-reference websites, protein sequences can be queried against the hidden Markov models (HMMMs) using HMMER homology search software (e.g., HMMER3, hmmer(dot)j anelia(dot)org/).

In some examples, the database may be NCBI's Conserved Domain Database (CDD) (Marchler-Bauer A, Lu S, Anderson J B, Chitsaz F, Derbyshire M K, DeWeese-Scott C, et al. (2011) CDD: a Conserved Domain Database for the functional annotation of proteins, nucleic Acids Res. 39: D225-D229).

In some examples, the database may be COG. The term “COG (clusters of orthologous groups)” may encompass a large collection of protein families classified according to their homologous relationships available at e.g. the NCBI COG website (www(dot)ncbi(dot)nlm(dot)nih(dot)gov/COG). Each COG comprises a group of proteins found to be orthologous across at least three lineages and likely corresponds to an ancient conserved domain [see e.g. Tatusov et al. Science 1997 Oct. 24; 278(5338):631-7; and Tatusov et al. nucleic Acids Res. 2000 Jan. 1; 28(1): 33-36].

The methods may further comprise filter false positives among the identified known defense genes.

The methods may further comprise, after the false positives of the known defense genes are filtered, identifying known defense systems. A defense system may comprise one or more defense proteins or nucleic acids involved in defense function. Examples of the known defense systems used in the methods include mobilome, a CRISPR system, Type I RM and McrBC system, BREX-associated system, Zorya system, Wadjet system, Druantia-associated system, Hachiman system, Lamassu system, Thoeris-like system, Gabija system, Septu system, pAgo system, Shedu system, Kiwa system, DUF499-DUF1156 system, and Toxin/antitoxin system.

The methods may further comprise recording (e.g., tabulating) candidate genes, which are genes within certain distance of a known defense system gene. The candidate genes may be on the 5′ side or the 3′ side of the defense system gene. For examples, the candidate genes may be within 50 kb, 40 kb, 30 kb, 20 kb, 18 kb, 16 kb, 14 kb, 12 kb, 10 kb, 9 kb, 8 kb, 7 kb, 6 kb, 5 kb, 4 kb, 3 kb, 2 kb, 1 kb, 900 bp, 800 bp, 700 bp, 600 bp, 500 bp, 400 bp, 300 bp, 200 bp, or 100 bp from the known defense system. In some examples, the candidate genes are within 10 kb of a defense system. In some cases, each of the candidate gene is called a seed.

The methods may further comprise, for each of the candidate gene, identifying homologs in the genomes. A homolog of the candidate gene may be a gene that share at least 50%, 60%, 70%, 80%, 90%, 95%, 99%, or 100% sequence identity with the candidate gene. In some examples, the homologs share at least 70% of sequence identity with the candidate genes.

In some cases, the homologs may have an E-value of 10⁻³ or lower, 10⁻⁴ or lower, 10⁻⁵ or lower, 10⁻⁶ or lower, 10⁻⁷ or lower, or 10⁻⁸ or lower. The Expect value or E-value refers to a parameter that describes the number of hits one can “expect” to see by chance when searching a database of a particular size. Essentially, the E-value describes the random background noise. For example, an E value of 1 assigned to a hit can be interpreted as meaning that in a database of the current size one might expect to see 1 match with a similar score simply by chance. The lower the E-value, or the closer it is to zero, the more “significant” the match (e.g., homology, identity) is.

The methods may further comprise selecting putative defense system genes from the candidate genes. The selected putative defense system genes may have at least a portion of the homologs in proximity to the known defense system genes. For example, a selected putative defense system genes may have at least 5%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, or at least 50% of its homologs. In some examples, a selected putative defense system genes may have at least 15% of the its homologs in proximity to the known defense system.

In some embodiments, the selection of putative defense system genes comprises selecting putative cassettes comprising multiple candidate genes. Each of the candidate genes in the putative cassette may have at least 5%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, or at least 50% of its homologs. In some examples, each of the candidate genes in the putative cassette may have at least 15% of the its homologs in proximity to the known defense system.

When a candidate gene or its homolog is in proximity to a known defense gene, the candidate gene or its homolog may be within 1000 nt, 900 nt, 800 nt, 700 nt, 600 nt, 500 nt, 400 nt, 300 nt, 200 nt, 100 nt, 80 nt, 60 nt, 40 nt, 20 nt, 10 nt, 5 nt, 4 nt, 3 nt, 2 nt, or 1 nt from the known defense gene.

### Validation of Identified Defense Systems

In some embodiments, the methods further comprise validating the selected putative defense systems and genes. The validation may be performed by introducing the putative defense system in host cells, infected the cells with virus (e.g., phages), and test phage infection efficiencies. Host cells introduced with a functional defense system may significantly suppress the phage infection efficiency. Examples of methods of validation include those described in Doron S. et al., Science. 2018 Mar. 2; 359(6379), Systematic discovery of antiphage defense systems in the microbial pangenome.

### Methods of Use

The defense systems herein may be introduced to host cells to manipulate the cells' function and activity. In some examples, the defense systems may be introduced to bacteria to manipulate their resistance to phage infection. In some embodiments, the defense systems may be introduced to eukaryotic cells to manipulate the function, structure, level, and/or expression of proteins or nucleic acids.

### Protection of Bacteria

In some embodiments, the defense systems may be introduced to bacteria or other host cells to increase the cells' resistance to an infection. In some cases, the defense systems may be used to protect bacterial fermentation from phage infection and contamination, which is a main cause of slow fermentation or complete starter failure. The lack of bacteria which survive adequately can result in milk products which do not have a desirable taste.

In some embodiments, the defense systems may be introduced to bacteria useful in the manufacture of dairy and fermentation processing such as, but not limited to, milk-derived products, such as cheeses, yogurt, fermented milk products, sour milks, and buttermilk. In some embodiments, the bacteria are useful as a part of the starter culture in the manufacture of dairy and fermentation processing. In some embodiments, the starter culture is a food grade starter culture. Examples of such bacteria include lactic acid bacteria, which encompass Gram positive, microaerophillic or anaerobic bacteria which ferment sugar with the production of acids including lactic acid as the predominantly produced acid, acetic acid, formic acid and propionic acid. Examples of the bacteria include Lactococcus species, Streptococcus species, Lactobacillus species, Leuconostoc species, Oenococcus species, Pediococcus species, Bifidobacterium species, and Propionibacterium species. In some embodiments, bacteria protected in a method of protecting bacteria from phage infection comprises bacteria selected from a Lactococcus species, a Streptococcus species, a Lactobacillus species, a Leuconostoc species, a Oenococcus species, a Pediococcus species, a Bifidobacterium, and a Propionibacterium species. In some embodiments, a method of protecting bacteria from phage infection comprises protecting a Lactococcus species of bacteria. In some embodiments a method of protecting bacteria from phage infection comprises protecting a Streptococcus species of bacteria. In some embodiments a method of protecting bacteria from phage infection comprises protecting a Lactobacillus species of bacteria. In some embodiments, a method of protecting bacteria from phage infection comprises protecting a Leuconostoc species of bacteria. In some embodiments, a method of protecting bacteria from phage infection comprises protecting a Oenococcus species of bacteria. In some embodiments, a method of protecting bacteria from phage infection comprises protecting a Pediococcus species of bacteria. In some embodiments, a method of protecting bacteria from phage infection comprises protecting a Bifidobacterium of bacteria. In some embodiments, a method of protecting bacteria from phage infection comprises protecting a Propionibacterium species of bacteria.

### Enhancing Bacteria Susceptibility to Infection

In some embodiments, the defense systems may be introduced to bacteria or other host cells to decrease the cells' resistance to an infection. In some examples, the defense system may be engineered to reduce or eliminate its defense function. In certain examples, one or more modulating agents that manipulate the function or level of the defense systems may be introduced to the host cells.

In some examples, the present disclosure provides methods of treating bacterial infection in a subject in need thereof, the method comprising administering to the subject a therapeutically effective amount of the anti-Defense System agent, thereby treating the bacterial infection in the subject. In some embodiments, there is provided the agent, for use in the treatment of bacterial infection in a subject in need thereof. In some examples, the present disclosure provides methods of generating cells as reagents that can be easily infected by phages. Such cells may be used as research tools in biotechnology.

### Engineered Cells

The present disclosure provides engineered cells comprising the systems and/or polynucleotides herein. In some cases, the cells may be where the plasmids and/or vesicles are produced. For example, the cells may be host cells, such as bacterial cells. In some examples, the cells may be eukaryotic cells, in which the systems are used for manipulating the function and other activities of the cells.

The cell may be a prokaryotic cell. The prokaryotic cell may be a bacterial cell. The prokaryotic cell may be an archaea cell. Examples of bacterial cells include those from the genus Escherichia, Bacillus, Lactobacillus, Rhodococcus, Rodhobacter, Synechococcus, Synechoystis, Pseudomonas, Psedoaltermonas, Stenotrophamonas, and Streptomyces. Examples of bacterial cells include Escherichia coli cells, Caulobacter crescentus cells, Rodhobacter sphaeroides cells, Psedoaltermonas haloplanktis cells. Suitable strains of bacterial include, but are not limited to BL21(DE3), DL21(DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue(DE3), BLR, C41(DE3), C43(DE3), Lemo21(DE3), Shuffle T7, ArcticExpress and ArticExpress (DE3).

The cell can be a eukaryotic cell. The eukaryotic cells may be those of or derived from a particular organism, such as a plant or a mammal, including human, or non-human eukaryote or animal or mammal as herein discussed, e.g., mouse, rat, rabbit, dog, livestock, or non-human mammal or primate. In some aspects the engineered cell can be a cell line. Examples of cell lines include C8161, CCRF-CEM, MOLT, mIMCD-3, NHDF, HeLa-S3, Huhl, Huh4, Huh7, HUVEC, HASMC, HEKn, HEKa, MiaPaCell, Pancl, PC-3, TF1, CTLL-2, C1R, Rath, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bc1-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalc1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

Further, the cell may be a fungus cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerervisiae, Kluyveromyces marxianus, or Issatchenkia orientalis cell. Other yeast cells may include without limitation Candida spp. (e.g., Candida albicans), Yarrowia spp. (e.g., Yarrowia hpolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains can include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In some aspects, the cell is a cell obtained from a subject. In some embodiments, the subject is a healthy or non-diseased subject.

In some embodiments, a cell transfected with one or more vectors described herein is used to establish a new cell line comprising one or more vector-derived sequences. The cells can be used to produce the engineered systems. In some embodiments, the engineered systems are produced, harvested, and delivered to a subject in need thereof. In some embodiments, the engineered cells are delivered to a subject. Other uses for the engineered cells are described elsewhere herein.

In some aspects, the present disclosure also provides tissues, organs, or subjects (e.g., animals, plants, etc.) comprising one or more cells described above.

### Engineered Animals

The present disclosure further provides engineered organisms that comprise the systems, polynucleotides, and/or vectors. The engineered organism, in some embodiments, can be an animal; for example, a mammal. In aspects, the organism is a non-human mammal. In an aspect, the invention provides a non-human eukaryotic organism; e.g., a multicellular eukaryotic organism, comprising a eukaryotic engineered cell according to any of the described embodiments. In other aspects, the invention provides a eukaryotic organism, preferably a multicellular eukaryotic organism, comprising a eukaryotic host cell according to any of the described embodiments. The engineered organism in some embodiments of these aspects may be an animal, for example, a mammal. In some embodiments, the engineered organism can be an arthropod such as an insect. In some embodiments, the engineered organism can be a farm or other production animals, including but not limited to pigs, goats, cattle, chickens, and sheep.

Various methods of generating transgenic animals that contain exogenous genetic material can be generated by various methods that will be appreciated by those of ordinary skill in the art. Such techniques include, but are not limited to, polynucleotide or virus microinjection into a pronucleus in a developing embryo, cell cytoplasm, or into the vasculature or blastoderm of a developing embryo (for example, in chickens); embryonic stem cell or other stem cell (e.g. pluripotent, multipotent, or induced pluripotent stem cell) manipulation (e.g. introduction of transgene or modification via gene editing); techniques utilizing a cre-lox approach, viral vectors, nuclear transfer, primoridial germ cell manipulation, spermatogonial manipulation. Many variations of these basic techniques have been done and are included within the scope of this disclosure. Exemplary methods for generating various transgenic animals can be found, for example, in any of the following, which are incorporated by reference as if expressed in their entirety: “Transgenic Animal Science: Principles and Methods” (1991) Charles River Laboratory; Hammer R. E, Pursel V. G, et al: Production of transgenic rabbits, sheep and pigs by microinjection. Nature 1985; 315(6021):680-683; Jaenisch R: Germ line integration and Mendelian transmission of the exogenous Moloney leukemia virus. Proc Natl Acad Sci.1976; 73:1260-1264; Brackett B G, Boranska W, Sawicki W, Koprowski: Uptake of heterologous genome by mammalian spermatozoa and its transfer to ova through fertilization. Proc Natl Acad Sci.1971; 68:353-357; Gordon J. W, Scangos G. A, Plotkin D. J, Barbosa J. A, Ruddle F. H: Genetic transformation of mouse embryos by microinjection of purified DNA. Proc Natl Acad Sci.1980; 77:179-184; Lavitrano M, Camaioni A, Fazio V. M, Dolci S, Farace M. G, Spadafora C: Sperm cells as vectors for introducing foreign DNA into eggs: genetic transformation of mice. Cell 1989; 57(5):717-723; Chang K, Qian J, et al: Effective generation of transgenic pigs and mice by linker based sperm-mediated gene transfer. BMC Biotechnol. 2002; 2(1):5; Perry A. C, Wakayama T, Kishikawa H, Kasai T, Okabe M, Toyoda Y, Yanagimachi R: Mammalian transgenesis by intracytoplasmic sperm injection. Science 1999; 284 (5417):1180-1183; Clark J, Whitelaw B: A future for transgenic livestock. Rev. Genet. 2003; 4(10):825-833; Bowen R. A: Efficient production of transgenic cattle by retroviral infection of early embryos. Reprod. Dev. 1995; 40(3):386-390; Shim H, Gutierrez-Adan A, Chen L. R, BonDurant R. H, Behboodi E, Anderson G. B: Isolation of pluripotent stem cells from cultured porcine primordial germ cells. Reprod. 1997; 57(5):1089-1095; Maclean, N: Animals with Novel Genes. Cambridge University Press. Cambridge, UK, 1995; Ebert, K. M, and Schindler J. E. S: Transgenic farm animals: Progress report. Theriogenology 1993; 39: 121-135; Gossler et al: Transgenesis by means of blastocyst-derived embryonic stem cell line, Proceedings of National Academic Science 1986; 83:9065-9069; Makoto Nagano, Clayton J. Brinster, et al: Transgenic mice produced by retroviral transduction of male germ-line stem cells. PNAS2001; 98(23):13090-13095; Alexander Baguisi et al: Production of goats by somatic cell nuclear transfer. Nature Biotechnology 1999; 17:456; Esponda P: Transfection of gametes. A method to generate transgenic animals. J. Morphol. 2005; 23(3):281-284; Andreas Sched, Zonia Larin, et al: A method for the generation of YAC transgenic mice by pronuclear microinjection. Nucleic Acids Research1993; 21(20):4783-4787; Ralph L. Brinster. Germline Stem Cell Transplantation and Transgenesis. Reproductive Biology Journal 2002; 296:2174; Hofmann A, Zakhartchenko V, et al: Generation of transgenic cattle by lentiviral gene transfer into oocytes. Reprod. 2004; 71(2):405-409; Sang H. M: Transgenics, chickens and therapeutic proteins. Vox Sanguinis. 2004; 87(2):S164-5166; Meade H. M, Echelard Y, et al: Expression of recombinant proteins in the milk of transgenic animals. In Gene expression systems: using nature for the art of expression. Academic Press, San Diego. 1999; 399-427; Rudolph N. S: Biopharmaceutical production in transgenic livestock. Trends Biotechnol. 1999; 17(9):367-374; Kuroiwa Y, Kasinathan P, et al: Cloned transchromosomic calves producing human immunoglobulin. Nature Biotechnol. 2002; 20(9):889-894; Swabson M. E, Martin M. J, et al: Production of functional human hemoglobin in transgenic swine. Biotechnology 1992; 10(5):557-559, Niemann H: Transgenic pigs expressing plant genes. natl Acad. Sci.2004; 101(19):7211-7212.

### Engineered Plants and Algae

The engineered organism, in some embodiments, can be a plant and algae that comprise the systems, polynucleotides, and/or vectors. In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants. In some embodiments, the engineered plant is a dicotyledonous plant belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. In some embodiments, the plant is a monocotyledonous plant such as one belonging to an order of the group of: Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g. those belonging to the orders Pinales, Ginkgoales, Cycadales, Araucariales, Cupressales and Gnetales. In some embodiments, the engineered plant can be a plant of a species included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

Specifically, the engineered plants are intended to include without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

The term plant also encompasses Algae, which are mainly photoautotrophs unified primarily by their lack of roots, leaves and other organs that characterize higher plants. Thus, in some embodiments, the modified organism is an algae. “Algae” and “algae cells,” include but are not limited to, algae or cells thereof selected from several eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). The term “algae” includes for example algae selected from Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

As noted above, part of the plant may be engineered to include and/or express one or more components of the engineered system described herein. As used herein, “plant tissue” refers to part of the plant and includes cells. The term “plant cell” as used herein refers to individual units of a living plant, either in an intact whole plant or in an isolated form grown in in vitro tissue cultures, on media or agar, in suspension in a growth media or buffer or as a part of higher organized unites, such as, for example, plant tissue, a plant organ, or a whole plant.

As used herein, “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

### Therapeutic and Diagnostic Applications

In another aspect, the present disclosure provides methods for treating diseases or conditions in a subject with the systems described herein. In some embodiments, the methods comprise administering one or more components of the systems, the polynucleotides, the vectors the cells, or any combination thereof, to a subject (e.g., a subject in need thereof). The systems may comprise or may cause production of therapeutic and/or diagnostic agents, such as the genetic modulating agents. in certain examples, the methods may comprise administering one or more cells comprising the vesicles or plasmids into a subject.

The diseases may be genetic diseases. Genetic diseases that can be treated are discussed in greater detail elsewhere herein. Other diseases include but are not limited to any of the following: cancer, Acubetivacter infections, actinomycosis, African sleeping sickness, AIDS/HIV, ameobiasis, Anaplasmosis, Angiostrongyliasis, Anisakiasis, Anthrax, Acranobacterium haemolyticum infection, Argentine hemorrhagic fever, Ascariasis, Aspergillosis, Astrovirus infection, Babesiosis, Bacterial meningitis, Bacterial pneumonia, Bacterial vaginosis, Bacteroides infection, balantidiasis, Bartonellosis, Baylisascaris infection, BK virus infection, Black Piedra, Blastocytosis, Blastomycosis, Bolivian hemorrhagic fever, Botulism, Brazilian hemorrhagic fever, brucellosis, Bubonic plague, Burkholderia infection, buruli ulcer, calicivirus invention, campylobacteriosis, Candidiasis, Capillariasis, Carrion's disease, Cat-scratch disease, cellulitis, Chagas Disease, Chancroid, Chickenpox, Chikungunya, Chlamydia, Chlamydia pneumoniae, Cholera, Chromoblastomycosis, Chytridiomycosis, Clonochiasis, Clostridium difficile colitis, Coccidioidomycosis, Colorado tick fever, rhinovirus/coronavirus infection (common cold), Cretzfeldt-Jakob disease, Crimean-congo hemorrhagic fever, Cryptococcosis, Cryptosporidiosis, Cutaneous larva migrans (CLM), cyclosporiasis, cysticercosis, cytomegalovirus infection, Dengue fever, Desmodesmus infection, Dientamoebiasis, Diphtheria, Diphylobothriasis, Dracunculiasis, Ebola, Echinococcosis, Ehrlichiosis, Enterobiasis, Enterococcus infection, Enterovirus infection, Epidemic typhus, Erthemia Infectisoum, Exanthem subitum, Fasciolasis, Fasciolopsiasis, fatal familial insomnia, filarisis, Clostridum perfingens infection, Fusobacterium infection, Gas gangrene (clostridial myonecrosis), Geotrichosis, Gerstmann-Straussler-Scheinker syndrome, Giardasis, Glanders, Gnathostomiasis, Gonorrhea, Granuloma inguinales, Group A streptococcal infection, Group B streptococcal infection, Haemophilus influenzae infection, Hand, foot, and mouth disease, hanta virus pulmonary syndrome, heartland virus disease, Helicobacter pylori infection, hemorrhagi fever with renal syndrome, Hendra virus infection, Hepatitis (all groups A, B, C, D, E), herpes simplex, histoplasmosis, hookworm infection, human bocavirus infection, human ewingii ehrlichiosis, Human granulocytic anaplasmosis, human metapneumovirus infection, human monocytic ehrlichiosis, human papilloma virus, Hymenolepiasis, Epstein-Barr infection, mononucleosis, influenza, isoporisis, Kawasaki disease, Kingell kingae infection, Kuru, Lasas fever, Legionellosis (Legionnaire's disease and Potomac Fever), Leishmaniasis, Leprosy, Leptospirosis, Listeriosis, Lyme disease, lymphatic filariasis, lymphocytic choriomeningitis, Malaria, Marburg hemorrhagic fever, measles, Middle East respiratory syndrome, Melioidosis, meningitis, Meningococcal disease, Metagonimiasis, Microsporidosis, Molluscum contagiosum, Monkeypox, Mumps, Murine typhus, Mycoplasma pneumonia, Mycoplasma genitalium infection, Mycetoma, Myiasis, Conjunctivitis, Nipah virus infection, Norovirus, Variant Creutzfeldt-Jakob disease, Nocardosis, Onchocerciasis, Opisthorchiasis, Paracoccidioidomycosis, Paragonimiasis, Pasteurellosis, Pediculosis capitis, Pediculosis corporis, Pediculosis pubis, pelvic inflammatory disease, pertussis, plague, pneumococcal infection, pneumocystis pneumonia, pneumonia, poliomyelitis, prevotella infection, primary amoebic meningoencephalitis, progressive multifocal leukoencephalopathy, Psittacosis, Qfever, rabies, relapsing fever, respiratory syncytial virus infection, rhinovirus infection, rickettsial infection, Rickettsia pox, Rift Valley Fever, Rocky Mountain Spotted Fever, Rotavirus infection, Rubella, Salmonellosis, SARS, Scabies, Scarlet fever, Schistosomiasis, Sepsis, Shigellosis, Shingles, Smallpox, Sporotrichosis, Staphylococcal infection (including MRSA), strongyloidiasis, subacute sclerosing panencephalitis, Syphilis, Taeniasis, tetanus, Trichophyton species infection, Tocariasis, Toxoplasmosis, Trachoma, Trichinosis, Trichuriasis, Tuberculosis, Tularemia, Typhoid Fever, Typhus Fever, Ureaplasma urealyticum infection, Valley fever, Venezuelan equine encephalitis, Venezuelan hemorrhagic fever, Vibrio species infection, Viral pneumonia, West Nile Fever, White Piedra, Yersinia pseudotuberculosis, Yersiniosis, Yellow fever, Zeaspora, Zika fever, Zygomycosis and combinations thereof.

Other diseases and disorders that can be treated using embodiments of the present invention include endocrine diseases (e.g. Type I and Type II diabetes, gestational diabetes, hypoglycemia. Glucagonoma, Goiter, Hyperthyroidism, hypothyroidism, thyroiditis, thyroid cancer, thyroid hormone resistance, parathyroid gland disorders, Osteoporosis, osteitis deformans, rickets, ostomalacia, hypopituitarism, pituitary tumors, etc.), skin conditions of infections and non-infection origin, eye diseases of infectious or non-infectious origin, gastrointestinal disorders of infectious or non-infectious origin, cardiovascular diseases of infectious or non-infectious origin, brain and neuron diseases of infectious or non-infectious origin, nervous system diseases of infectious or non-infectious origin, muscle diseases of infectious or non-infectious origin, bone diseases of infectious or non-infectious origin, reproductive system diseases of infectious or non-infectious origin, renal system diseases of infectious or non-infectious origin, blood diseases of infectious or non-infectious origin, lymphatic system diseases of infectious or non-infectious origin, immune system diseases of infectious or non-infectious origin, mental-illness of infectious or non-infectious origin and the like.

In some embodiments, the disease may be neuronal diseases. The systems herein may be delivered to neuronal cells or related cells for treating such diseases. Examples of diseases and cells include those described in Bergen J M et al., Nonviral Approaches for Neuronal Delivery of Nucleic Acids, Pharm Res. 2008 May; 25(5): 983-998.

### Pharmaceutical Compositions

The systems, polynucleotides, vectors, and cells herein may be formulated as pharmaceutical compositions. A pharmaceutical composition may comprise an excipient, such as a pharmaceutically acceptable carrier, that is conventional in the art and that is suitable for administration to cells or to a subject.

In certain embodiments, the methods of the disclosure include administering to a subject in need thereof an effective amount (e.g., therapeutically effective amount or prophylactically effective amount) of the treatments provided herein. Such treatment may be supplemented with other known treatments, such as surgery on the subject. In certain embodiments, the surgery is strictureplasty, resection (e.g., bowel resection, colon resection), colectomy, surgery for abscesses and fistulas, proctocolectomy, restorative proctocolectomy, vaginal surgery, cataract surgery, or a combination thereof.

The term “pharmaceutically acceptable” as used throughout this specification is consistent with the art and means compatible with the other ingredients of a pharmaceutical composition and not deleterious to the recipient thereof. As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical compositions can be applied parenterally, rectally, orally or topically. For example, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infuse. The skilled person will understand that compositions which are to be administered orally or topically will usually not comprise cells, although it may be envisioned for oral compositions to also comprise cells, for example when gastro-intestinal tract indications are treated. Each of the cells or active components (e.g., modulants, immunomodulants, antigens) as discussed herein may be administered by the same route or may be administered by a different route. By means of example, and without limitation, cells may be administered parenterally and other active components may be administered orally. In some cases, the composition or pharmaceutical composition may by intramuscular injection. In some cases, the composition or pharmaceutical composition may by intravascular injection.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregelatinized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

The pharmaceutical compositions may comprise one or more pharmaceutically acceptable salts. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methyl sulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agents, cells, agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing an inflammatory response (e.g., a person who is genetically predisposed or predisposed to allergies or a person having a disease characterized by episodes of inflammation) may receive prophylactic treatment to inhibit or delay symptoms of the disease.

### Vaccines

The systems, vesicles, plasmids, and cells may be used as vaccines. In some examples, the vesicles may comprise molecules capable of eliciting T cell and B cell immune responses. In some examples, the vesicles may not replicate once delivered in a target cell.

### Bioproduction

The engineered system molecules, vectors, engineered cells, and/or engineered systems can be used for bioproduction of various molecules including engineered systems. In some embodiments, the engineered cells can be used in an in vivo (e.g. a modified animal or plant), in vitro, or ex vivo cell system to produce engineered systems. As previously mentioned, the engineered system molecules, vectors, engineered cells, and/or engineered systems can be used to make a modified animal that can produce engineered systems. In some embodiments, the animal can be engineered to produce engineered systems in one or more bodily fluids or product (e.g. an egg as in the case of modified avians). As previously mentioned, the engineered system molecules, vectors, engineered cells, and/or engineered systems can be used to make a modified plant that can produce engineered systems. In some embodiments, the plant can be engineered to produce engineered systems in one or more parts of the plant. In some embodiments, production can be in a harvestable portion of the plant.

In some embodiments, the objective can be to make and/or harvest a particular molecule from a producer cell. This can be useful for generating and harvesting molecules that are otherwise difficult to generate and/or harvest outside of a cell or via other processes and techniques. In some embodiments, the molecule is one that is naturally produced by the producer cell (which can be an engineered cell). In some embodiments, the producer cell can be engineered to increase production of one or more endogenous molecules. In some embodiments, the producer cell is engineered to produce an exogenous molecule. In some embodiments, endogenous and/or exogenous molecules produced can be packaged into engineered systems, which can be subsequently harvested from the producer cell. The molecules can then be further harvested from the engineered systems. Methods of purifying engineered systems are described elsewhere herein and will be appreciated by those of ordinary skill in the art. Similarly, methods of harvesting the molecules from the engineered systems will be appreciated by those of ordinary skill in the art.

In some cases, endogenous producer cell molecules or exogenous molecules of interest are normally secreted by the producer cell. Packaging these into engineered systems prior to secretion followed by subsequent purification of the engineered systems carrying the packaged endogenous molecule can be an alternative to obtaining conditioned media to obtain these normally secreted endogenous molecules.

The systems (e.g., the systems comprising ATPase(s) and adenosine deaminase(s) described herein) may be used to modify polynucleotides in vitro, in cells, and in vivo. Examples of applications, e.g., in plants, fungi, animals, therapeutic and diagnostic applications, include those described in International Patent Publication Nos. WO 2019/071048 (e.g. paragraphs [0528]-[0837]), WO 2019/084063 (e.g., paragraphs [0676]-[0892]), which are incorporated by reference herein in their entireties.

### Delivery

The one or more components of the systems herein may be introduced to cells for expression. Examples of methods of introducing the components into cell include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). Physical methods of introducing polynucleotides may also be used. Examples of such methods include injection of a solution containing the polynucleotides, bombardment by particles covered by the polynucleotides, soaking a cell, tissue sample or organism in a solution of the polynucleotides, or electroporation of cell membranes in the presence of the polynucleotides. Examples of delivery methods and vehicles include viruses, nanoparticles, exosomes, nanoclews, liposomes, lipids (e.g., LNPs), supercharged proteins, cell permeabilizing peptides, and implantable devices. The nucleic acids, proteins and other molecules, as well as cells described herein may be delivered to cells, tissues, organs, or subjects using methods described in paragraphs [00117] to [00278] of Feng Zhang et al., (WO2016106236A1), which is incorporated by reference herein in its entirety.

### EXAMPLES

### Example 1—Identification of Bacterial Defense Systems

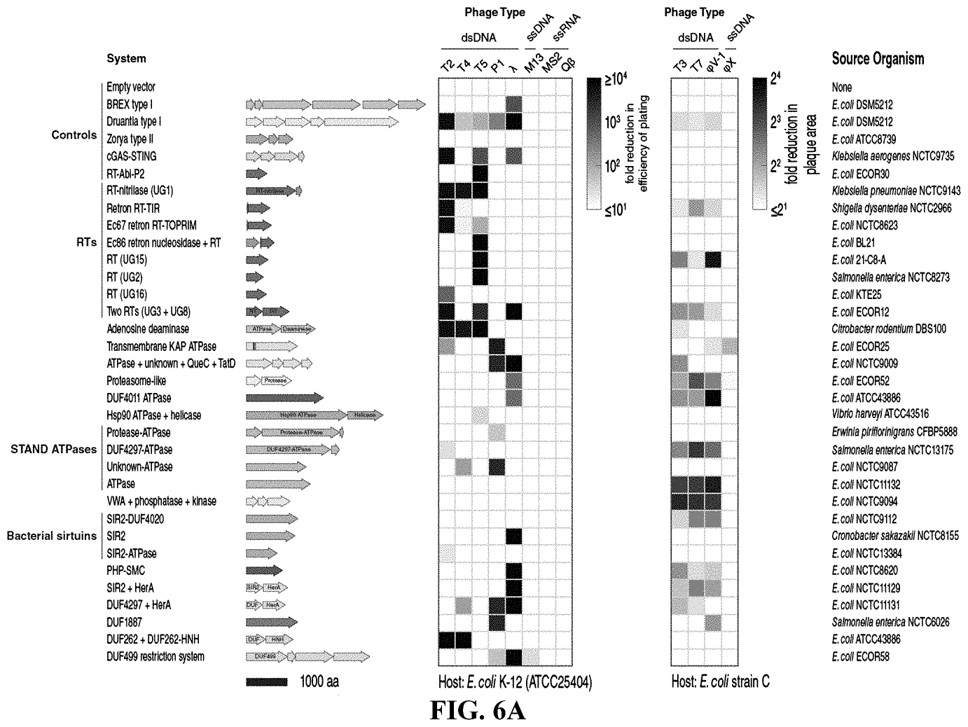

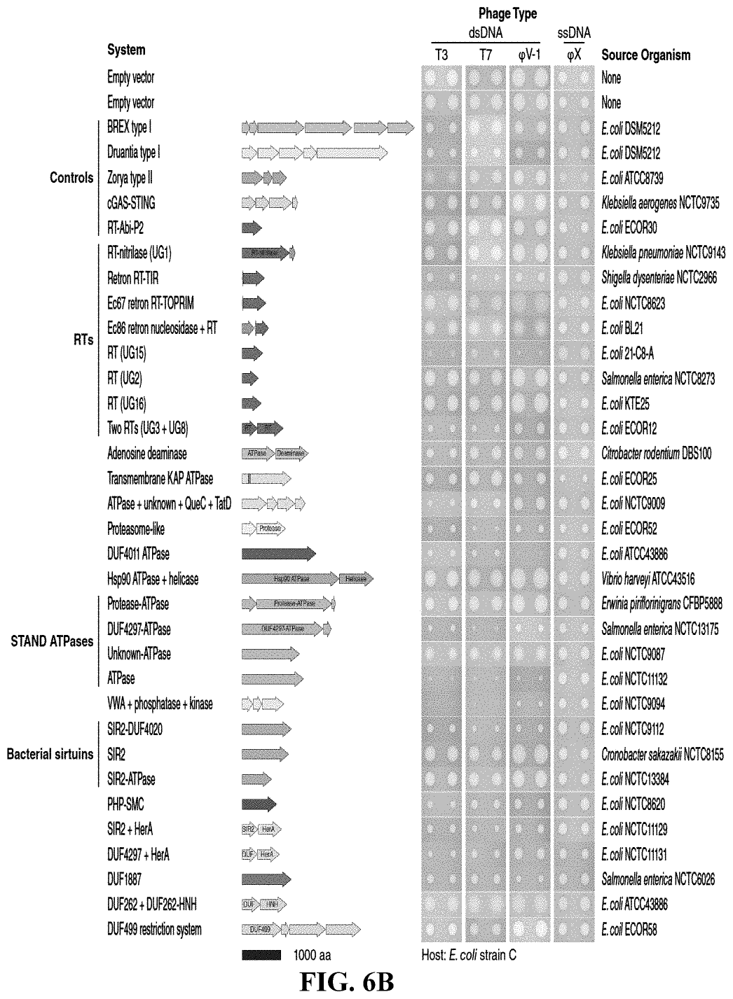

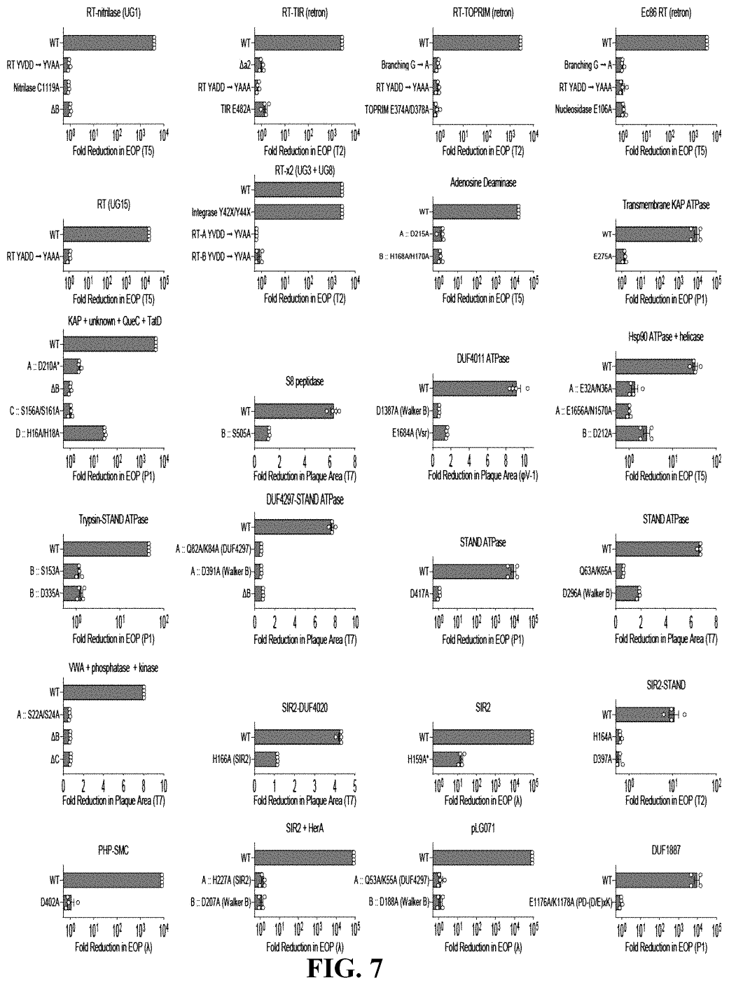

Bacterial defense systems were identified using method outlined in FIG. 5, FIGS. 6A-6B show the examples of the identified bacterial defense systems, their domain structures, and their effects on phage growth. Selected identified bacterial defense systems and mutated forms were tested for their effects on phage growth (FIG. 7).

### Example 2—Diverse Enzymatic Functions Mediate Antiviral Immunity in Prokaryotes

Bacteria and archaea possess multiple defense systems to protect against attacking viruses and other foreign genetic elements through a variety of mechanisms, including sequence-specific endonucleases and toxin-antitoxin systems. Here, using a systematic approach to identify defense-associated genes in bacterial and archaeal genomes, Applicants identified a diverse set of putative defense gene cassettes that remain functionally uncharacterized. Applicants heterologously reconstituted 50 of these cassettes in Escherichia coli, demonstrating that 29 of them mediated defense against specific bacteriophages. These new defense systems include retrons; a widespread family of reverse transcriptases with unusual domain associations; and STAND ATPases, which are homologs of essential eukaryotic apoptosis effectors but whose role in prokaryotes has remained enigmatic. In addition, Applicants demonstrated that a two-gene system containing a divergent adenosine deaminase mediates RNA editing upon exposure to phage, representing a novel mechanism of defense. The discovery of these novel defense systems highlighted the immense untapped diversity of molecular functions employed by microbes in their wars against viruses and provides clues to the evolutionary origins of microbial immune mechanisms.

Bacterial and archaeal viruses are the most abundant, and possibly the most diverse, biological entities on earth (Cobián Güemes et al., 2016; Suttle, 2013). To defend against the incessant and varied virus attacks, prokaryotes have evolved multiple, diverse antivirus defense systems. These include the adaptive immune systems CRISPR-Cas, which provide immunity by memorizing past infection events (Hille et al., 2018), and a variety of innate immune systems, such as restriction-modification (RM)-based systems, including DNA phosphorothioation, DPD, DISARM (Ofir et al., 2018), and BREX (Goldfarb et al., 2015; Gordeeva et al., 2019), which target specific, pre-defined sequences within the phage DNA; abortive infection (Abi) systems, which induce altruistic cell dormancy or death upon phage infection; and additional systems with mechanisms that have not yet been investigated (Doron et al. 2018). Antivirus defense systems range in complexity from a single small protein (e.g., certain types of Abi systems) to large cassettes of eight or more proteins acting in concert (e.g., type I and type III CRISPR-Cas systems).

The arms race between microbes and viruses is a powerful evolutionary force that sculpts the host genomes. A distinctive outcome of this process is the modularity of defense systems, whereby components of one system are often recruited by other systems. For example, restriction-modification enzymes have been found in association with a number of additional proteins, leading to expanded defense systems, such as DISARM (Ofir et al., 2018). Toxin-antitoxin systems are particularly prone to swapping, resulting in nearly every possible combination of toxin and antitoxin (Makarova et al., 2013). Another key feature of the evolution of microbial anti-parasite defense is the persistent exchange of components between defense systems and mobile genetic elements (Koonin et al., 2019). In particular, nucleases encoded by both transposons and toxin-antitoxin modules apparently have been recruited for roles in CRISPR-Cas systems, and conversely, components of CRISPR-Cas systems have been recruited by mobile genetic elements for antidefense and other functions, such as RNA-guided transpositions (Faure et al., 2019; Klompe et al., 2019; Strecker et al., 2019). The extensive modularity and baroque evolutionary patterns of defense systems yield extraordinary diversity and highlight the potential for discovery of additional systems with novel mechanisms.

### Domain-Independent Identification of Uncharacterized Defense Systems

A distinctive property of anti-phage defense genes is their tendency to cluster together within defense ‘islands’ in bacterial and archaeal genomes (Makarova et al., 2013; Makarova et al., 2011). As a consequence, an uncharacterized gene whose homologs consistently occur next to, for instance, restriction-modification genes has an increased probability of being a new defense gene (Shmakov et al., 2019; Shmakov et al., 2018). A recent analysis (Doron et al., 2018) identified and validated 10 new defense systems, based on the requirement that each (putative) system contain at least one annotated protein domain that is enriched within defense islands.

To test whether additional unknown systems existed which either lack annotated domains, or only contain domains that are typically non-defense but have been co-opted in specific instances to perform defensive functions, Applicants developed an expanded computational approach in which putative novel systems were identified independent of domain annotations (FIG. 8A). Applicants analyzed all 174,080 bacterial and archaeal genomes available in Genbank as of November 2018, encoding a total of 620 million proteins. To identify candidate novel defense systems, Applicants first compiled a list of all proteins within 10 kb or 10 open reading frames of known defense systems (see Methods). This list (n=6×10⁵ after redundancy reduction) was a mix of novel defense genes with many non-defense genes. For each entry in the list (‘seed’), Applicants identified all homologs within the original set of genomes with an alignment coverage of at least 70% and an E-value of 10⁻⁵ or lower. Each detected homolog was then assessed for its proximity to a known defense system. For each seed, if the fraction of homologs within 5 kb of 5 genes of a known defense system (‘defense association score’) (Shmakov et al., 2019) was sufficiency high, the seed was retained for further analysis (see Methods). For each retained seed, the gene neighborhoods of 30 representative homologs were examined to identify conserved operons that contain the seed gene and putatively constitute a minimal intact defense system.

To determine an appropriate cutoff for the defense association score, Applicants performed the same analysis for a selected set of seeds from known systems. From this analysis, a value of 0.15 was chosen because >90% of the known seeds had a score higher than this value (FIG. 8B). Applying this threshold to the novel seeds resulted in a final list of 1.5×10⁴ defense gene candidates (10.5% of all seeds; minimum 50 identified homologs) (FIG. 8C). This analysis suggested that uncharacterized defense systems substantially outnumbered the currently known ones. Furthermore, the defense-enriched seeds included a diversity of identified enzymatic activities, including those that had not been previously implicated in antivirus immunity.

### Candidate Defense Systems Exhibited Antivirus Activity in a Heterologous System

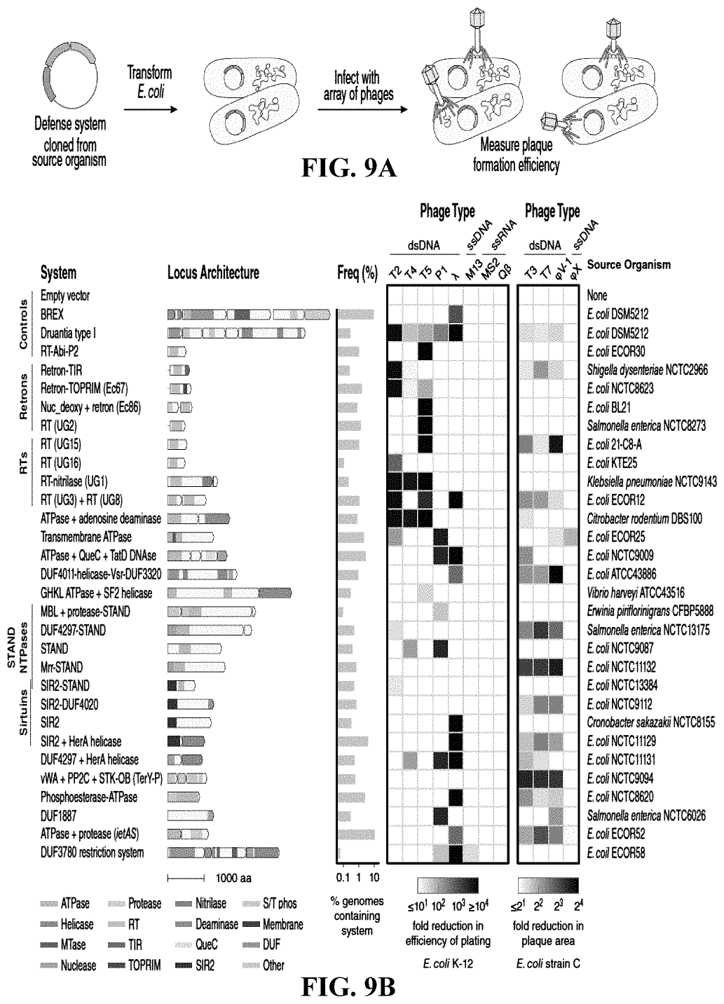

Applicants selected 50 candidate defense systems to test experimentally by heterologous reconstitution in E. coli. Candidate systems were prioritized for testing based on the following criteria: presence of identified molecular functions not previously implicated in defense; broad phylogenetic distribution; and for multi-gene systems, conservation of component genes. For each system, 1-4 homologs were selected and cloned from the source organism into the low-copy vector pACYC and transformed into E. coli (FIG. 9A). BREX type I (Goldfarb et al., 2015; Gordeeva et al., 2019), Druantia type I (Doron et al., 2018), and the abortive infection reverse transcriptase RT-Abi-P2 (Odegrip et al., 2006) were included as positive controls. Each system was then challenged with a diverse panel of coliphages with dsDNA, ssDNA, or ssRNA genomes, and phage sensitivity was compared to that observed with an empty vector control.

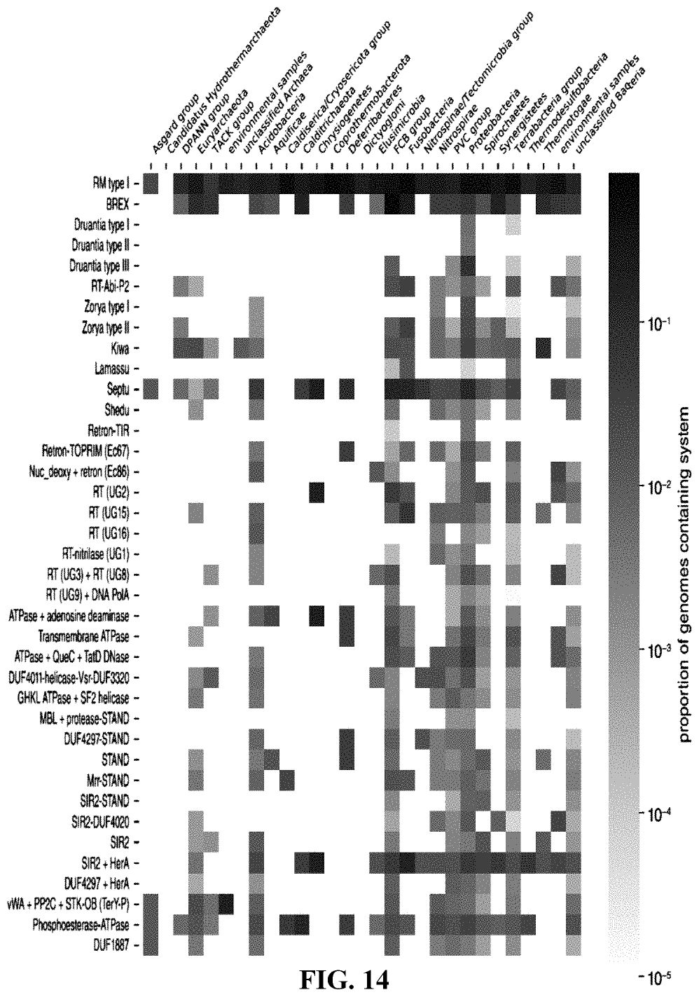

Applicants observed anti-phage activity in at least one homolog for 29 out of the 50 tested candidates (58%). The most active representative in each of these 29 systems was further tested with an expanded panel of phages in two E. coli strains (FIG. 9B). All 29 systems were active against at least one dsDNA phage; three were active against ssDNA phages (M13 or φX174); and none were active against ssRNA phages (MS2 and Q(3). Phage specificity was typically narrow and varied widely across systems. In addition, the abundance of these systems within sequenced genomes spans two orders of magnitude, ranging from ˜0.1% to ˜10% of the genomes (FIG. 9B and FIG. 14).

RADAR Contained a Divergent Adenosine Deaminase that Edits RNA in Response to Phage Infection

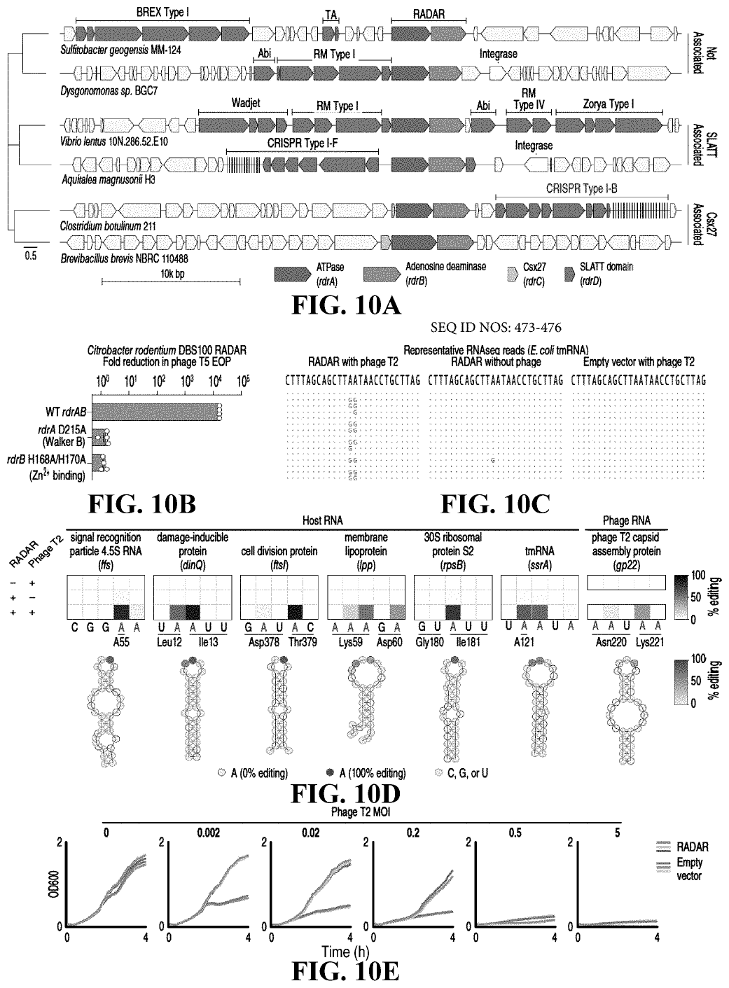

One of the validated systems was a two-gene cassette consisting of a KAP-family ATPase (˜900 residues) and a divergent adenosine deaminase (˜900 residues); this system was active against dsDNA phages T2, T3, T4, and T5. Applicants focused on this system for further investigation because deaminase activity had not previously been implicated in anti-phage defense. These systems appear in diverse defense contexts, adjacent to CRISPR, BREX, RM, Zorya, and Wadjet, and form three distinct subtypes (FIG. 10A). In some cases, this system had the ATPase and deaminase only, but some variants also included a small membrane protein, either a SLATT domain (Burroughs et al., 2015) or the type VI-B CRISPR ancillary gene csx27 (Makarova et al., 2019). Mutations in either the ATPase Walker B motif or in the putative Zn2+-binding H×H motif of the deaminase abolished defense activity (FIG. 10B).

Applicants further tested whether it acted on nucleic acids. Indeed, whole-transcriptome deep sequencing showed an enrichment of A to G substitutions in sequencing reads at specific sites in the presence of phage, whereas C, G, or U bases were not affected (FIG. 10C), consistent with base editing of adenosine to inosine. Editing occurred when both the defense system and the phage were present. In this experiment, expression of the defense system without the phage resulted in a near-baseline level of editing, and no editing was detected in the absence of the system. The editing sites were distributed throughout the E. coli transcriptome as well as the phage transcriptome (FIG. 10D). RNA secondary structure analysis indicated a characteristic stem-loop structure at strong editing sites; specific adenosines in loops were edited with up to ˜90% frequency, whereas adenosines within the stem were not edited within the limit of detection.

Based on these results, Applicants named this system phage restriction by an adenosine deaminase acting on RNA (RADAR). Growth kinetics at varying phage multiplicity of infection (MOI) revealed a threshold MOI above which RADAR-expressing cells had a lower OD600 compared to the empty vector control, suggestive of RADAR-mediated growth arrest (FIG. 10E). Collectively, these results are consistent with an abortive infection mechanism that is activated by phage.

### A Widespread Family of RT-Containing Defense Systems

The defense systems identified by the pipeline herein included a diverse family of reverse transcriptases (RTs). Although RTs are typical components of diverse mobile retroelements as well as retro-transcribing viruses, some RTs encoded in bacterial genomes show no evidence of mobility (Zimmerly and Wu, 2015). Two of these RTs have been previously shown to play a role in anti-phage defense, namely RT-Cas1, which mediated acquisition of CRISPR spacers from RNA via reverse transcription (Silas et al., 2016), and RT-Abi, a set of abortive infection genes that catalyzed untemplated dNTP polymerization in vitro (Emond et al., 1997; Odegrip et al., 2006; Wang et al., 2011).

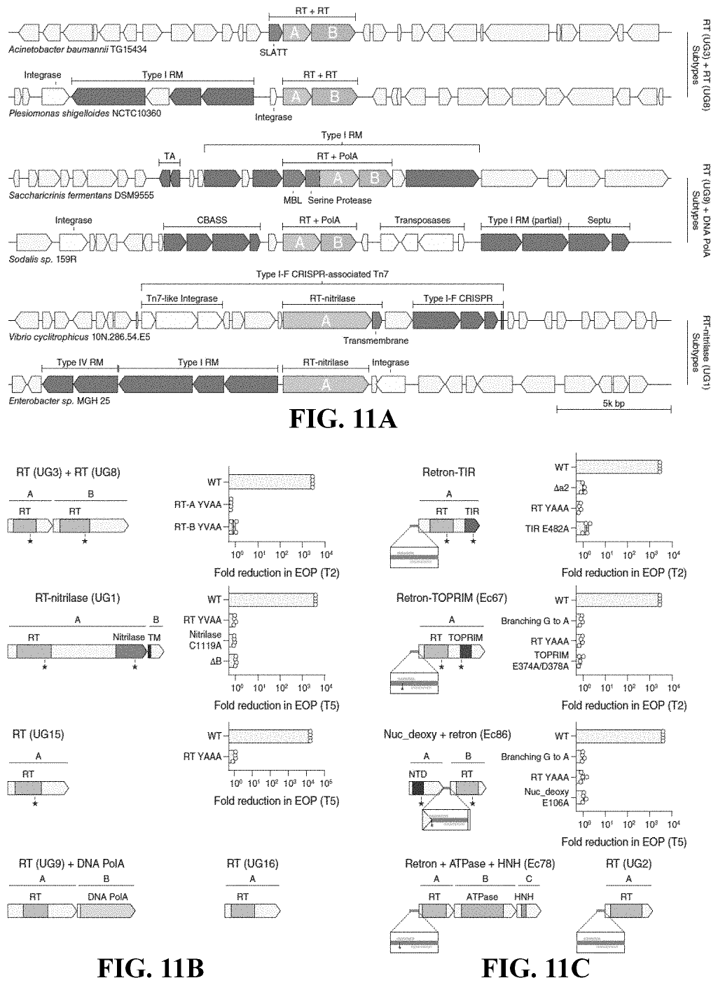

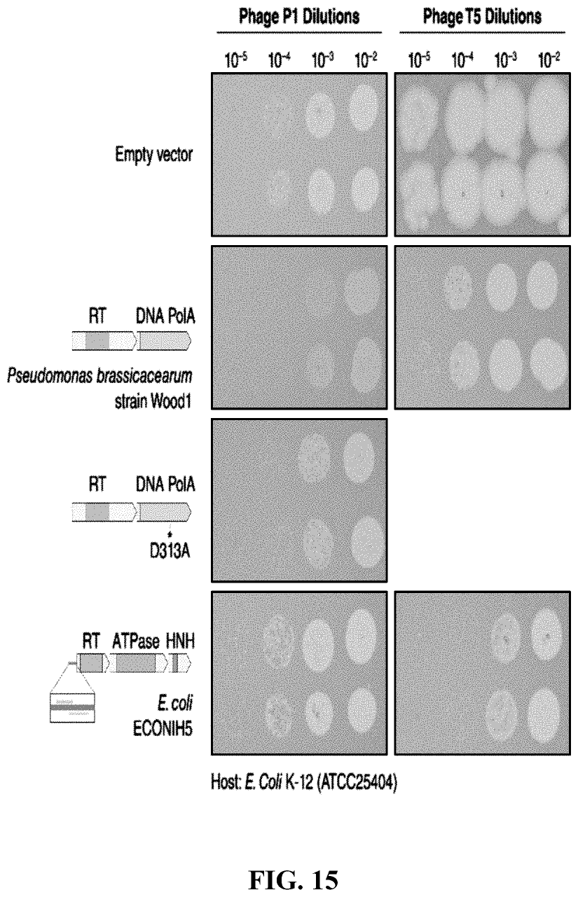

Recent computational analyses have revealed a vast diversity of bacterial RTs, including 16 ‘unknown groups’ (UGs) that either remained functionally uncharacterized, or were identified to perform metabolic roles (Kojima and Kanehisa, 2008; Simon and Zimmerly, 2008; Toro and Nisa-Martinez, 2014; Zimmerly and Wu, 2015). Many of these RTs were independently identified by the computational pipeline herein, suggesting that they might represent a widespread family of uncharacterized defense genes. Applicants found that at least 7 of these RT groups (UG1, UG2, UG3, UG8, UG9, UG15, and UG16) provided robust protection against dsDNA phages (FIG. 9B), and mutations in the (Y/F)×DD (SEQ ID NOS: 1-2) active site of the RTs abolished activity (FIG. 11A-11C). Many of these RTs contained an uncharacterized C-terminal domain, and some were fused to or associated with required enzymatic domains that had not been previously implicated in anti-phage defense, including a nitrilase-family C—N hydrolase and a family A DNA polymerase (FIGS. 11A, B and FIG. 15).

### Retrons Mediated Anti-Phage Defense

Applicants also identified defense functions for a group of retrons, a distinct class of RTs that produce extrachromosomal satellite DNA (multi-copy single-stranded DNA, msDNA) by reverse transcribing a segment of the 5′ region of its own mRNA (Lampson et al., 2005). Retron cDNA is covalently linked to an internal guanosine of the RNA via a 2′-5′ phosphodiester bond. Retrons had been harnessed for bacterial genome engineering (Farzadfard and Lu, 2014), but their native biological function had remained unknown. Applicants found that the original E. coli retrons Ec67 (Lampson et al., 1989) and Ec86 (Lim and Maas, 1989), as well as the Ec78 retron (Lima and Lim, 1997) and a novel TIR domain-associated retron, mediated defense against dsDNA phages. In addition, the absence of additional domains typical for group II introns in the UG2 group, together with the presence of a large upstream region that formed a identified highly structured RNA, suggested that UG2 was yet another retron-like element. Mutations in the (Y/F)×DD (SEQ ID NOS: 1-2) active site of the RT, as well as a G to A substitution at the branching guanosine, abolished activity, indicating that the defense function depends on msDNA synthesis. Notably, these retrons were associated with other domains, including TOPRIM (topoisomerase-primase) (Aravind et al., 1998) and TIR (Tol/interleukin 1 receptor) domains, that were required for activity (FIG. 11C). The TOPRIM domain can possess nuclease activity (Aravind et al., 1998) whereas the TIR domain can be a NAD+ hydrolase that is involved in programmed cell death pathways in animals and plants (Horsefield et al., 2019).

### Additional Molecular Functions

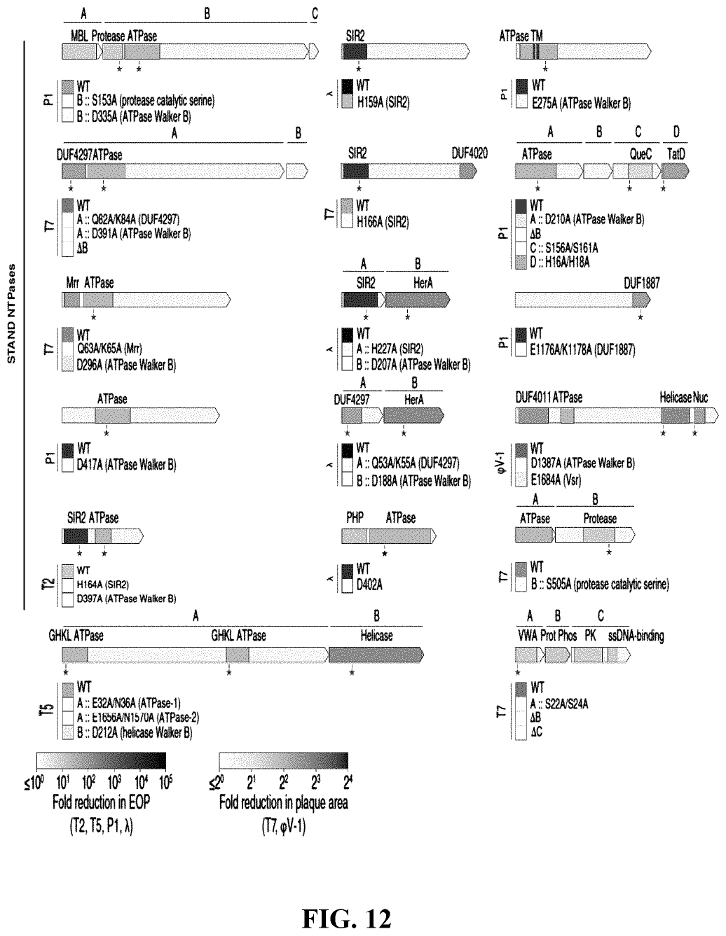

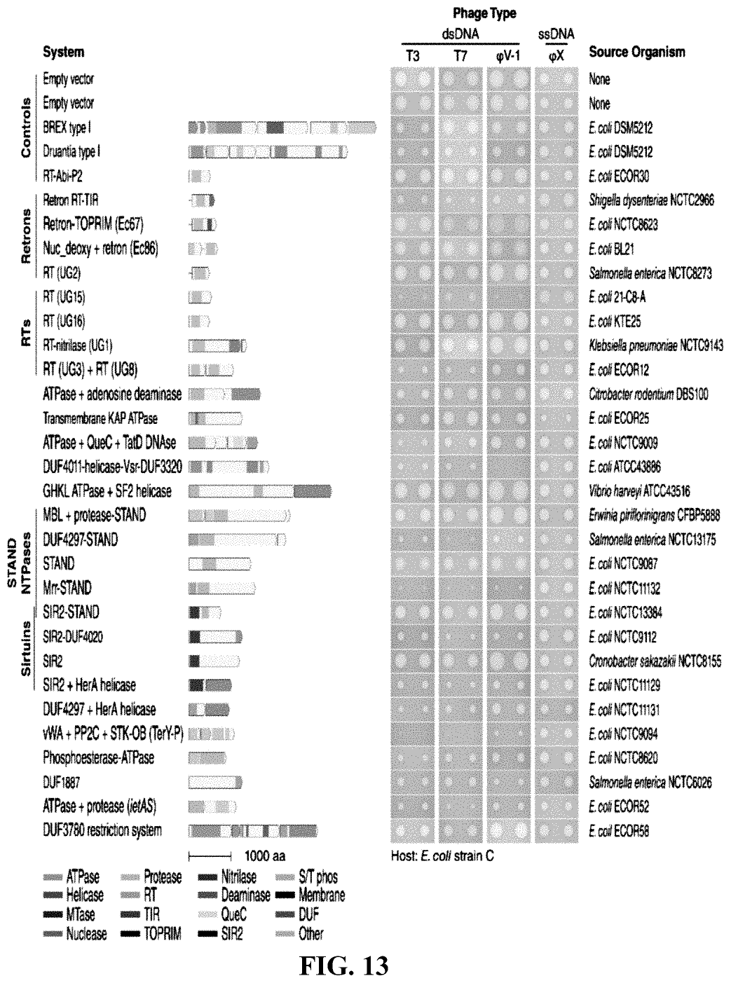

Applicants identified other defense systems with diverse molecular functions, including a three-gene cassette containing a von Willebrand factor A (vWA) domain protein, a PP2C-like serine/threonine protein phosphatase, and a serine/threonine protein kinase provided strong protection against T7-like phages (T3, T7, and φV-1). In this experiment, all three genes were required for activity (FIG. 12). This system, termed the TerY-phosphorylation triad (TerY-P), was previously analyzed computationally in the context of Ter-dependent stress response systems (Anantharaman et al., 2012) and can operate as a phosphorylation switch that couples the activities of the kinase and the phosphatase.

Four systems contained an N-terminal SIR2 (sirtuin) deacetylase domain (FIG. 12), which was present in the Thoeris system (Doron et al., 2018) and had also been detected in the same neighborhoods with prokaryotic Argonaute proteins (Makarova et al., 2009), but had not been functionally characterized in prokaryotes. Additionally, a large 1300 residue P-loop ATPase containing two transmembrane helices inserted into the ATPase domain, similarly to the KAP family ATPases (Aravind et al., 2004), protected against both dsDNA and ssDNA phages.

Applicants also demonstrated defense function for several identified NTPases of the STAND (signal transduction ATPases with numerous associated domains) superfamily (FIG. 12). This expansive superfamily consists of multidomain proteins that include eukaryotic ATPases and GTPases involved in programmed cell death and various forms of signal transduction (Danot et al., 2009; Leipe et al., 2004). Typically, STAND NTPases contain a C-terminal helical sensor that, upon target recognition, induces oligomerization via ATP or GTP hydrolysis, leading to activation of the N-terminal effector domain. The functions of prokaryotic STAND NTPases remain poorly characterized. Those few for which experimental data are available contain a helix-turn-helix domain and have been shown to regulate transcription (Danot et al., 2009). Several identified STAND NTPases were active against dsDNA phages (FIG. 9B); these proteins contained different putative effector domains, including DUF4297 (a putative PD(D/E)×K-family nuclease that is also present in the Lamassu defense system (Doron et al., 2018)), an Mrr-like nuclease, SIR2, a trypsin-like serine protease, and an uncharacterized helical domain.

The findings described here substantially expanded the space of protein domains, molecular functions, and their interactions that are employed by bacteria in anti-phage defense. Some of these functions, in particular RNA editing, had not been previously implicated in defense mechanisms. The high success rate of the identification of defense systems based solely on the evolutionary conservation of the proximity to previously identified defense genes validated the defense island concept (Makarova et al., 2013; Makarova et al., 2011) and demonstrated its growing utility at the time of rapid expansion of sequence databases.

Despite similarities in domain architectures among some of the identified defense systems, their phage specificities differed substantially. The molecular basis of such narrow specificity remained to be uncovered, but these observations emphasized the importance of multiple defense systems for the survival of prokaryotes in the incessant arms race with viruses. Furthermore, these results were compatible with the concept of distributed microbial immunity, according to which defense systems encoded in different genomes collectively protect microbial communities from the diverse viromes they confront. The remarkable variability of the discovered defense systems implied that their sensor and effector components were involved in diverse molecular interactions. Several of the identified defense systems incorporated molecular functions from typically non-defense sources, highlighting the versatility of activities that were recruited for antiviral defense. The notable cases in point include the RNA deaminase activity of the RADAR system, as well as reverse transcriptases of different families, in particular retrons. The demonstration of the defense functions for multiple RTs that were generally associated with mobile genetic elements was consistent with the ‘guns for hire’ paradigm whereby enzymes are shuttled between MGE and defense systems during microbial evolution (Koonin et al., 2019).

The discovered defense systems can be characterized mechanistically, e.g., by mutating the catalytic residues. Applicants showed here that the respective enzymatic components were functionally important. Many of these systems can function via an abortive infection mechanism, e.g., by causing growth arrest or programmed cell death in the infected hosts as demonstrated here for the RADAR system. In particular, this can be the mode of action of STAND NTPases, homologs of essential eukaryotic programmed cell death effectors, whose role in prokaryotes has long remained enigmatic (Koonin and Aravind, 2002; Leipe et al., 2004). In addition, the membrane-associated ATPase can function analogously to the STAND NTPases to which they are distantly related (Aravind et al., 2004).

Many of the identified defense systems contained enzymatic activities as well as identified sensor components that had not been previously detected in defense contexts, suggesting the possibility of reengineering for novel biotechnology applications. Further experimental characterization of these systems, as well as others Applicants identified computationally, can be expected to greatly expand the repertoire of such functions.

### Methods

Detection of known antivirus defense systems. All bacterial and archaeal genomes (n=174,080) were downloaded from Genbank (ftp://ftp.ncbi.nih.gov/genomes/genbank/) in November 2018. For genomes where gene annotations were incomplete or missing, genes were identified using Prodigal (Hyatt et al., 2010). Known defense-related protein domains were annotated using RPSBLAST version 2.8.1 from a set of position-specific scoring matrices curated from the NCBI Conserved Domain Database (CDD) (Doron et al., 2018; Makarova et al., 2011; Marchler-Bauer et al., 2017; Punta et al., 2012). To reduce the false positive rate, a multi-gene system containing a ubiquitous protein domain was required to include two or more of its component genes in close proximity. For example, the type I restriction-modification endonuclease hsdR was called as a defense gene only if the corresponding methylase (hsdM) or specificity protein (hsdS) was also encoded in the vicinity. Toxin-antitoxin systems were excluded from the set of known defense systems due to their overall low enrichment within defense islands.

Candidate novel defense genes. All translated protein-coding sequences within either 10 kb or 10 genes of known defense systems (whichever was greater), including the components of the known defense systems themselves, were compiled into a preliminary list (n=8.7×10⁶). Highly similar sequences (at least 98% sequence identity and coverage) were discarded using the linclust option in MMseqs2 (Steinegger and Riding, 2017, 2018) with parameters—min-seq-id 0.98-c 0.98, resulting in a reduced list of 2.5×10⁶ sequences. A second round of redundancy elimination was then applied to this reduced list, using the default cluster option in MMSeqs2, yielding a final list of 6.0×10⁵ candidate sequences.

Scoring candidate genes for defense enrichment. For each of the 6.0×10⁵ candidate genes, a ‘defense enrichment score’ was computed as (number of homologs in proximity to one or more known defense systems)/(total number of homologs). A gene was considered to be located in proximity to a known defense system if it occurred no more than 5 kb or 5 genes away from the locus encoding that system. Candidate sequences with a defense enrichment score of 0.15 or higher were retained for subsequent analysis, with the exception of mobilome components (such as transposons), toxin-antitoxin, or abortive infection components, which were discarded. This cut-off was chosen because more than 90% of the known defense genes scored higher than this value. To identify homologs of the candidate proteins, all 6.2×10⁸ proteins in the original set of Genbank genomes were tabulated, and highly similar proteins (at least 98% sequence identity and coverage) were removed using linclust, resulting in a reduced list of 1.3×10⁸ proteins. Each seed sequence was then searched against this non-redundant protein sequence database using MiMseqs2. To qualify as homologs, matches were required to have a minimum coverage of 70% and a maximum E value of 10⁻⁵ (parameters—coy-mode 0-c 0.7-e 0.00001).

From genes to defense systems. For each defense-enriched candidate protein, the gene neighborhoods of 30 homologs in proximity to known defense genes were randomly selected and examined on a case by case basis, in order to determine whether the candidate was a stand-alone defense gene system or a member of a conserved multi-gene cassette. Protein domains were identified using HHpred, and the resulting identification were used to infer the involvement of the respective proteins in the activity of the respective identified defense system (Zimmermann et al., 2018).

Abundance estimation of defense systems. To estimate the abundance of each validated defense system within the microbial pangenome, Applicants downloaded n=205214 genomes available in Genbank as of August 2019. For each defense system, initial protein sequence seeds of the signature genes were taken from experimentally validated loci. Initial seeds were aligned and converted into HMM profiles. Applicants then used a constrained 2 iteration HMM profile search to generate highly specific HMM profiles and retrieve related systems as follows. Each ORF of size 150aa or greater with one or more hits was searched against all HMM profiles using HMMER3.1 and assigned to the profile that had the highest scoring match. For each system, ORFs with profile hits with less than 500 bp of intergenic distance on the same strand were grouped into candidate loci. For multi-protein systems, a putative locus was considered a hit if every signature gene profile for the system had a match in the locus with a bitscore of at least 25. For single gene systems, a locus was considered a hit if the protein had a match to the system's single signature gene profile with a bit score of at least 50 and an alignment coverage of at least 70%. Signature proteins from the identified systems were separately clustered at 50% identity using MMseqs2 and subsequently aligned using MAFFT. The alignments were used to create a new set of signature gene profiles as input to the next iteration. For BREX and Type I RM, Applicants used preexisting pfam profiles for the signature genes in place of iterative HMM profile searching. The final abundance was calculated as the number of system hits divided by the number of genomes (n).

Bacteria and phage strains. Phages T2, T3, T4, T5, T7, P1, λ, φV-1, M13, φX174, MS2, and Qβ, as well as host E. coli strains K-12 (ATCC25404) and C (ATCC13706), were obtained from the American Type Culture Collection (ATCC). The genome of phage φV-1, originally isolated from a measles vaccine (Milstien et al., 1977; Petricciani et al., 1973), was sequenced and found to be 92% similar to enterobacteria phage 285P, a T7-like phage (Xu et al., 2014).

Cloning. To facilitate experimental validation using coliphages, the source organism of each candidate defense system was chosen to be as similar as possible to E. coli, in particular, from other strains of E. coli whenever possible. Candidate defense systems were cloned into a variant of the low-copy plasmid pACYC184 containing 7 synonymous mutations in the chloramphenicol resistance gene to remove restriction sites. When possible, genomic DNA from source organisms was obtained from ATCC, NCTC, or DSMZ, and the genes of interest were amplified with Q5 (New England Biolabs) or Phusion Flash (Thermo Scientific) polymerase, using primers with 5′ ends homologous to the ends of the plasmid backbone. Plasmids were assembled using the NEBuilder HiFi DNA Assembly mix (New England Biolabs). When the source organism was not readily available from public culture collections, genes were chemically synthesized (GenScript) with optional human codon optimization of the open reading frames. When possible, the native promoter was retained. For some source organisms outside of Enterobacteriaceae, or when the candidate system was operonized with other upstream genes, the system was placed under a bla or lac promoter.

Sequence verification of plasmids. The full sequences of all plasmids were verified by high-throughput sequencing. To prepare sequencing libraries, 25-50 ng of each plasmid was mixed with purified Tn5 transposome loaded with Illumina adapters and incubated at 55° C. for 10 min in the presence of 5 mM MgCl2 and 10 mM TAPS buffer (Picelli et al., 2014). The quantity of Tn5 was titrated to generate an average fragment size of ˜100-400 bp. Tagmentation reactions were subsequently treated with 0.5 volumes of 0.1% sodium dodecyl sulfate for 5 min at room temperature and amplified with KAPA HiFi HotStart polymerase using primers containing 8 nt i7 and i5 index barcodes. Barcoded amplicons were sequenced on a MiSeq (Illumina) with at least 150 cycles for the forward read. Reads were aligned to the reference plasmid sequence by the Geneious read mapper, and error-free plasmids were retained for subsequent experiments.

Competent cell production. E. coli strains K-12 and C were cultured in ZymoBroth with 25 μg/mL chloramphenicol and made competent using Mix & Go buffers (Zymo) according to the manufacturer's recommended protocol.

Phage plaque assays. E. coli host strains were grown to saturation at 37° C. in Luria Broth (LB). To 10 mL top agar (10 g/L tryptone, 5 g/L yeast extract, 10 g/L NaCl, 7 g/L agar) was added chloramphenicol (final concentration 25 μg/mL) and 526 μL E. coli culture, and the mixture was poured on 10 cm LB-agar plates containing 25 μg/mL chloramphenicol. For phages T2, T4, T5, P1, λ, M13, MS2, and Qβ, dilutions of phage in phosphate buffered saline were spotted on the plates, and plaque counts were recorded after overnight incubation at 37° C. If individual plaques were too small to be counted, the most concentrated dilution at which no plaque formation was visible was recorded as having a single plaque. For phages T3, T7, φV-1, and φX174, a total of 3 μL of phage containing 5×106 virions was spotted, and the area of the plaque was measured after incubation at 37° C. for 68 hr.

Phage cultivation. Phages T2, T3, T4, T7, φV-1, M13, φX174, MS2, and Qβ were propagated in liquid culture. The host E. coli strain for each phage was grown to an OD600 of 0.2-0.4 at 37° C. in LB and infected with a slab of top agar containing phage plaque from a previous lysis. Cultures were grown overnight at 37° C. with 250 rpm agitation. Phages T5, P1, and λ, were propagated by the double agar overlay method; after overnight incubation at 37° C., plaques were scraped in LB. For both liquid culture and double agar overlay, phage samples were centrifuged to pellet cellular debris, and the supernatant was filtered through with a 0.22 μm sterile filter.

Whole transcriptome sequencing. E. coli ATCC25404, containing either an empty vector or the candidate defense system, was grown to log phase in LB and diluted to an OD600 of 0.2. The culture was then split into two tubes, one of which was infected with phage T2 at an estimated MOI of 2. Both subcultures were incubated at 37° C. for 1 hr with 250 rpm agitation. RNA was extracted using TRIzol Reagent (Thermo Fisher Scientific) and treated with DNAse I, followed by a RiboMinus ribosomal RNA depletion kit (Thermo). Sequencing libraries were prepared using NEB Ultra II directional RNAseq library prep kit (New England Biolabs) and paired-end sequenced (2×75 cycles) with a NextSeq (Illumina). Adapter sequences were trimmed from sequencing reads using CutAdapt (with parameters—trim-n-q 20-m 20-a AGATCGGAAGAGC-A AGATCGGAAGAGC (SEQ ID NO: 472)), and trimmed reads were aligned to the E. coli MG1655 reference genome using the Geneious read mapper.

RNA secondary structure. Minimum free energy RNA secondary structures were generated using the Turner (2004) energy parameters at 37° C. (Turner and Mathews, 2010).

E. coli growth kinetics. Cells were grown to log phase in LB and diluted to an OD600 of 0.2. Cultures were infected with phage T2 at varying MOI at grown at 37° C., and the OD600 was measured every 2 min for a total duration of 4 hr on a Synergy Neo2 plate reader (BioTek).
Anantharaman, V., Iyer, L. M., and Aravind, L. (2012). Ter-dependent stress response systems: novel pathways related to metal sensing, production of a nucleoside-like metabolite, and DNA-processing. Mol Biosyst 8, 3142-3165.
Aravind, L., Iyer, L. M., Leipe, D. D., and Koonin, E. V. (2004). A novel family of P-loop NTPases with an unusual phyletic distribution and transmembrane segments inserted within the NTPase domain. Genome Biol 5, R30.
Aravind, L., Leipe, D. D., and Koonin, E. V. (1998). Toprim—a conserved catalytic domain in type IA and II topoisomerases, DnaG-type primases, OLD family nucleases and RecR proteins. Nucleic Acids Res 26, 4205-4213.
Burroughs, A. M., Zhang, D., Schïffer, D. E., Iyer, L. M., and Aravind, L. (2015). Comparative genomic analyses reveal a vast, novel network of nucleotide-centric systems in biological conflicts, immunity and signaling. Nucleic Acids Res 43, 10633-10654.
Cobián Güemes, A. G., Youle, M., Cantú, V. A., Felts, B., Nulton, J., and Rohwer, F. (2016). Viruses as Winners in the Game of Life. Annu Rev Virol 3, 197-214.
Danot, O., Marquenet, E., Vidal-Ingigliardi, D., and Richet, E. (2009). Wheel of Life, Wheel of Death: A Mechanistic Insight into Signaling by STAND Proteins. Structure 17, 172-182.
Doron, S., Melamed, S., Ofir, G., Leavitt, A., Lopatina, A., Keren, M., Amitai, G., and Sorek, R. (2018). Systematic discovery of antiphage defense systems in the microbial pangenome. Science 359.
Emond, E., Holler, B. J., Boucher, I., Vandenbergh, P. A., Vedamuthu, E. R., Kondo, J. K., and Moineau, S. (1997). Phenotypic and genetic characterization of the bacteriophage abortive infection mechanism AbiK from Lactococcus lactis. Appl Environ Microbiol 63, 1274-1283.
Farzadfard, F., and Lu, T. K. (2014). Synthetic biology. Genomically encoded analog memory with precise in vivo DNA writing in living cell populations. Science 346, 1256272.
Faure, G., Shmakov, S. A., Yan, W. X., Cheng, D. R., Scott, D. A., Peters, J. E., Makarova, K. S., and Koonin, E. V. (2019). CRISPR-Cas in mobile genetic elements: counter-defence and beyond. Nat Rev Microbiol 17, 513-525.
Goldfarb, T., Sberro, H., Weinstock, E., Cohen, O., Doron, S., Charpak-Amikam, Y., Afik, S., Ofir, G., and Sorek, R. (2015). BREX is a novel phage resistance system widespread in microbial genomes. EMBO J 34, 169-183.
Gordeeva, J., Morozova, N., Sierro, N., Isaev, A., Sinkunas, T., Tsvetkova, K., Matlashov, M., Truncaite, L., Morgan, R. D., Ivanov, N. V., et al. (2019). BREX system of Escherichia coli distinguishes self from non-self by methylation of a specific DNA site. Nucleic Acids Res 47, 253-265.
Hille, F., Richter, H., Wong, S. P., Bratovič, M., Ressel, S., and Charpentier, E. (2018). The Biology of CRISPR-Cas: Backward and Forward. Cell 172, 1239-1259.
Horsefield, S., Burdett, H., Zhang, X., Manik, M. K., Shi, Y., Chen, J., Qi, T., Gilley, J., Lai, J. S., Rank, M. X., et al. (2019). NAD. Science 365, 793-799.
Hyatt, D., Chen, G. L., Locascio, P. F., Land, M. L., Larimer, F. W., and Hauser, L. J. (2010). Prodigal: prokaryotic gene recognition and translation initiation site identification. BMC Bioinformatics 11, 119.
Klompe, S. E., Vo, P. L. H., Halpin-Healy, T. S., and Sternberg, S. H. (2019). Transposon-encoded CRISPR-Cas systems direct RNA-guided DNA integration. Nature 571, 219-225.
Kojima, K. K., and Kanehisa, M. (2008). Systematic survey for novel types of prokaryotic retroelements based on gene neighborhood and protein architecture. Mol Biol Evol 25, 1395-1404.
Koonin, E. V., and Aravind, L. (2002). Origin and evolution of eukaryotic apoptosis: the bacterial connection. Cell Death Differ 9, 394-404.
Koonin, E. V., Makarova, K. S., Wolf, Y. I., and Krupovic, M. (2019). Evolutionary entanglement of mobile genetic elements and host defence systems: guns for hire. Nat Rev Genet.
Lampson, B. C., Inouye, M., and Inouye, S. (2005). Retrons, msDNA, and the bacterial genome. Cytogenet Genome Res 110, 491-499.
Lampson, B. C., Sun, J., Hsu, M. Y., Vallejo-Ramirez, J., Inouye, S., and Inouye, M. (1989). Reverse transcriptase in a clinical strain of Escherichia coli: production of branched RNA-linked msDNA. Science 243, 1033-1038.
Leipe, D. D., Koonin, E. V., and Aravind, L. (2004). STAND, a class of P-loop NTPases including animal and plant regulators of programmed cell death: multiple, complex domain architectures, unusual phyletic patterns, and evolution by horizontal gene transfer. J Mol Biol 343, 1-28.
Lim, D., and Maas, W. K. (1989). Reverse transcriptase-dependent synthesis of a covalently linked, branched DNA-RNA compound in E. coli B. Cell 56, 891-904.
Lima, T. M., and Lim, D. (1997). A novel retron that produces RNA-less msDNA in Escherichia coli using reverse transcriptase. Plasmid 38, 25-33.
Makarova, K. S., Gao, L., Zhang, F., and Koonin, E. V. (2019). Unexpected connections between type VI-B CRISPR-Cas systems, bacterial natural competence, ubiquitin signaling network and DNA modification through a distinct family of membrane proteins. FEMS Microbiol Lett 366.
Makarova, K. S., Wolf, Y. I., and Koonin, E. V. (2013). Comparative genomics of defense systems in archaea and bacteria. Nucleic Acids Res 41, 4360-4377.
Makarova, K. S., Wolf, Y. I., Snir, S., and Koonin, E. V. (2011). Defense islands in bacterial and archaeal genomes and prediction of novel defense systems. J Bacteriol 193, 6039-6056.
Makarova, K. S., Wolf, Y. I., van der Oost, J., and Koonin, E. V. (2009). Prokaryotic homologs of Argonaute proteins are predicted to function as key components of a novel system of defense against mobile genetic elements. Biol Direct 4, 29.
Marchler-Bauer, A., Bo, Y., Han, L., He, J., Lanczycki, C. J., Lu, S., Chitsaz, F., Derbyshire, M. K., Geer, R. C., Gonzales, N. R., et al. (2017). CDD/SPARCLE: functional classification of proteins via subfamily domain architectures. Nucleic Acids Res 45, D200-D203.
Milstien, J. B., Walker, J. R., and Petricciani, J. C. (1977). Bacteriophages in live virus vaccines: lack of evidence for effects on the genome of rhesus monkeys. Science 197, 469-470.
Odegrip, R., Nilsson, A. S., and Haggard-Ljungquist, E. (2006). Identification of a gene encoding a functional reverse transcriptase within a highly variable locus in the P2-like coliphages. J Bacteriol 188, 1643-1647.
Ofir, G., Melamed, S., Sberro, H., Mukamel, Z., Silverman, S., Yaakov, G., Doron, S., and Sorek, R. (2018). DISARM is a widespread bacterial defence system with broad anti-phage activities. Nat Microbiol 3, 90-98.
Petricciani, J. C., Chu, F. C., Johnson, J. B., and Meyer, H. M. (1973). Bacteriophages in live virus vaccines. Proc Soc Exp Biol Med 144, 789-792.
Picelli, S., Björklund, A. K., Reinius, B., Sagasser, S., Winberg, G., and Sandberg, R. (2014). Tn5 transposase and tagmentation procedures for massively scaled sequencing projects. Genome Res 24, 2033-2040.
Punta, M., Coggill, P. C., Eberhardt, R. Y., Mistry, J., Tate, J., Boursnell, C., Pang, N., Forslund, K., Ceric, G., Clements, J., et al. (2012). The Pfam protein families database. Nucleic Acids Res 40, D290-301.
Shmakov, S. A., Faure, G., Makarova, K. S., Wolf, Y. I., Severinov, K. V., and Koonin, E. V. (2019). Systematic prediction of functionally linked genes in bacterial and archaeal genomes. Nat Protoc 14, 3013-3031.
Shmakov, S. A., Makarova, K. S., Wolf, Y. I., Severinov, K. V., and Koonin, E. V. (2018). Systematic prediction of genes functionally linked to CRISPR-Cas systems by gene neighborhood analysis. Proc Natl Acad Sci USA 115, E5307-E5316.
Silas, S., Mohr, G., Sidote, D. J., Markham, L. M., Sanchez-Amat, A., Bhaya, D., Lambowitz, A. M., and Fire, A. Z. (2016). Direct CRISPR spacer acquisition from RNA by a natural reverse transcriptase-Cas1 fusion protein. Science 351, aad4234.
Simon, D. M., and Zimmerly, S. (2008). A diversity of uncharacterized reverse transcriptases in bacteria. Nucleic Acids Res 36, 7219-7229.
Steinegger, M., and Soding, J. (2017). MMseqs2 enables sensitive protein sequence searching for the analysis of massive data sets. Nat Biotechnol 35, 1026-1028.
Steinegger, M., and Soding, J. (2018). Clustering huge protein sequence sets in linear time. Nat Commun 9, 2542.
Strecker, J., Ladha, A., Gardner, Z., Schmid-Burgk, J. L., Makarova, K. S., Koonin, E. V., and Zhang, F. (2019). RNA-guided DNA insertion with CRISPR-associated transposases. Science 365, 48-53.
Suttle, C. A. (2013). Viruses: unlocking the greatest biodiversity on Earth. Genome 56, 542-544.
Toro, N., and Nisa-Martinez, R. (2014). Comprehensive phylogenetic analysis of bacterial reverse transcriptases. PLoS One 9, e114083.
Turner, D. H., and Mathews, D. H. (2010). NNDB: the nearest neighbor parameter database for predicting stability of nucleic acid secondary structure. Nucleic Acids Res 38, D280-282.
Wang, C., Villion, M., Semper, C., Coros, C., Moineau, S., and Zimmerly, S. (2011). A reverse transcriptase-related protein mediates phage resistance and polymerizes untemplated DNA in vitro. Nucleic Acids Res 39, 7620-7629.
Xu, B., Ma, X., Xiong, H., and Li, Y. (2014). Complete genome sequence of 285P, a novel T7-like polyvalent E. coli bacteriophage. Virus Genes 48, 528-533.
Zimmerly, S., and Wu, L. (2015). An Unexplored Diversity of Reverse Transcriptases in Bacteria. Microbiol Spectr 3, MDNA3-0058-2014.
Zimmermann, L., Stephens, A., Nam, S. Z., Rau, D., Kithler, J., Lozajic, M., Gabler, F., Soding, J., Lupas, A. N., and Alva, V. (2018). A Completely Reimplemented MPI Bioinformatics Toolkit with a New HHpred Server at its Core. J Mol Biol 430, 2237-2243.

TABLE 5
Source organism strains of validated defense systems.

| # | System | Genes | Organism | Strain | Promoter |
|---|---|---|---|---|---|
| | BREX type I | 6 | E. coli | DSM5212 | Native |
| | Druantia type I | 5 | E. coli | DSM5212 | Native |
| | RT-Abi-P2 | 1 | E. coli | ECOR30 | Native |
| 1 | RT_retron-TIR | 1 | Shigella | NCTC2966 | Native |
| | | | dysenteriae | | |
| 2 | RT_retron-TOPRIM (Ec67) | 1 | E. coli | NCTC8623 | Native |
| 3 | Nuc_deoxy + RT_retron (Ec86) | 2 | E. coli | BL21 | Native |
| 4 | RT_UG2 | 1 | Salmonella | NCTC8273 | Native |
| | | | enterica | | |
| 5 | RT_UG15 | 1 | E. coli | 21-C8-A | Native |
| 6 | RT_UG16 | 1 | E. coli | KTE25 | Native |
| 7 | RT_UG1-nitrilase | 2 | Klebsiella | NCTC9143 | Native |
| | | | pneumoniae | | |
| 8 | RT_UG3 + RT_UG8 | 2 | E. coli | ECOR12 | Native |
| 9 | ATPase_AAA + Ada | 2 | Citrobacter | ATCC51459 | Native |
| | | | rodentium | | |
| 10 | ATPase_KAP_TM | 1 | E. coli | ECOR25 | Native |
| 11 | ATPase_KAP + QueC + DNase_TatD | 4 | E. coli | NCTC9009 | Native |
| 12 | DUF4011-Helicase_SF1_Dna2- | 1 | E. coli | ATCC43886 | Native |
| | Nuclease_Vsr-DUF3320 | | | | |
| 13 | ATPase_GHKL + Helicase_SF2_HepA | 2 | Vibrio harveyi | ATCC43516 | bla |
| 14 | MBL + Protease_S1-ATPase_STAND | 3 | Erwinia | CFBP5888 | bla |
| | | | piriflorinigrans | | |
| 15 | DUF4297-ATPase_STAND | 2 | Salmonella | NCTC13175 | Native |
| | | | enterica | | |
| 16 | ATPase_STAND | 1 | E. coli | NCTC9087 | Native |
| 17 | Nuclease_Mrr-ATPase_STAND | 1 | E. coli | NCTC11132 | Native |
| 18 | SIR2-ATPase_STAND | 1 | E. coli | NCTC13384 | Native |
| 19 | SIR2-DUF4020 | 1 | E. coli | NCTC9112 | Native |
| 20 | SIR2 | 1 | Cronobacter | NCTC8155 | Native |
| | | | sakazakii | | |
| 21 | SIR2 + Helicase_HerA | 2 | E. coli | NCTC11129 | Native |
| 22 | Nuclease_DUF4297 + Helicase_HerA | 2 | E. coli | NCTC11131 | Native |
| 23 | vWA + phosphatase_PP2C + STK-IB | 3 | E. coli | NCTC9094 | Native |
| 24 | Phosphoesterase_PHP-ATPase_SMC | 1 | E. coli | NCTC8620 | Native |
| 25 | Nuclease_DUF1887 | 1 | Salmonella | NCTC6026 | Native |
| | | | enterica | | |
| 26 | ATPase_AAA + Protease_S8 | 2 | E. coli | ECOR52 | Native |
| 27 | ATPase_DUF499 + DUF3780 + | 4 | E. coli | ECOR58 | Native |
| | Methylase_DUF1156 + Nuclease_PLD- | | | | |
| | Helicase_HepA | | | | |
| 28 | RT_IG9 + DNA Po1A | 2 | Pseodomonas | Wood1 | lac |
| | | | brassicacearum | | Native |
| 29 | RT_retron _ ATPase_AAA + HNH (Ec78) | 3 | E. coli | ECONIH5 | Native |

TABLE 6
PCR primers used to amplify genomic DNA source
organisms containing validated defense systems.

| # | Primer | Sequence |
|---|---|---|
| BREX | Fwd | gctaacttacattaattgcgttgcgcaACAGCACCACGTTCATCTTCC |
| type I | | (SEQ ID NO: 98) |
| | Rev | ccaaggggttatgctagttattgcgGTTCATTAAAATAGTTACTACGTTAATTCACACCC |
| | | (SEQ ID NO: 99) |
| Druantia | Fwd | gctaacttacattaattgcgttgcgcaGGTGAACGTTTGGTTGATAGGG |
| type I | | (SEQ ID NO: 100) |
| | Rev | ccaaggggttatgctagttattgcgCTCAATGGGCATAATTTTACATTGTGC |
| | | (SEQ ID NO: 101) |
| RT-Abi-P2 | Fwd | gctaacttacattaattgcgttgcgcaACATCCCGTCATCATGCCATC |
| | | (SEQ ID NO: 102) |
| | Rev | ccaaggggttatgctagttattgcgCTCCTCGGAATAGAATGTTATGTTCG |
| | | (SEQ ID NO: 103) |
| 1 | Synthesized | |
| 2 | Fwd | gctaacttacattaattgcgttgcgcaCGCGCTATCACGTAAAATAGGC |
| | | (SEQ ID NO: 104) |
| | Rev | ccaaggggttatgctagttattgcgCGAAAAATCAGCCTTAGCGTTCATAAC |
| | | (SEQ ID NO: 105) |
| 3 | Fwd | gctaacttacattaattgcgttgcgcaGCTCATGTTATGCATGTGCATG |
| | | (SEQ ID NO: 106) |
| | Rev | ccaaggggttatgctagttattgcgATTAGGTCTTCGCTTTATTTAAAGGGTTC |
| | | (SEQ ID NO: 107) |
| 4 | Synthesized | |
| 5 | Synthesized | |
| 6 | Synthesized | |
| 7 | Fwd | gagctaacttacattaattgcgttgcgcaGTCCTTAAACACGACAAAACCTGTG |
| | | (SEQ ID NO: 108) |
| | Rev | cccaaggggttatgctagttattgcgCGCAATGTAACACCCACCC |
| | | (SEQ ID NO: 109) |
| 8 | Fwd | gctaacttacattaattgcgttgcgcaTCTCAACTTCCCCAAATGTCCG |
| | | (SEQ ID NO: 110) |
| | Rev | cccaaggggttatgctagttattgcgTTAGCAAAATACGCCCACGAAGTC |
| | | (SEQ ID NO: 111) |
| 9 | Fwd | gctaacttacattaattgcgttgcgcaGAGGATTTATGCACAAAATCCTGATGC |
| | | (SEQ ID NO: 112) |
| | Rev | ccaaggggttatgctagttattgcgGATTTAATCTGTTGTTCCGAACGG |
| | | (SEQ ID NO: 113) |
| 10 | Fwd | gctaacttacattaattgcgttgcgcaACCGTGCTGGCATGTTTTTAC |
| | | (SEQ ID NO: 114) |
| | Rev | ccaaggggttatgctagttattgcgAGGAAGATCCGTGACCAGGAG |
| | | (SEQ ID NO: 115) |
| 11 | Fwd | gctaacttacattaattgcgttgcgcaGAAATTATTTGGAATGGATGATGGCG |
| | | (SEQ ID NO: 116) |
| | Rev | ccaaggggttatgctagttattgcgACTTCTACCTCCCTTTAGAAAAGTTAATG |
| | | (SEQ ID NO: 117) |
| 12 | Fwd | gctaacttacattaattgcgttgcgcaCGGATTGAATCTGTTTATGAAATTTGGCTG |
| | | (SEQ ID NO: 118) |
| | Rev | ccaaggggttatgctagttattgcgCCGACAGTTGTCACTGTTCTTATTACC |
| | | (SEQ ID NO: 119) |
| 13 | Fwd | ccctgataaatgcttcaataatattgaaaaaggaagagtATGGCGGGTGCTTCAATAGAC |
| | | (SEQ ID NO: 120) |
| | Rev | cccaaggggttatgctagttattgcgTTAGTTACTTGCTTTGTAGAATACCGTTAATGG |
| | | (SEQ ID NO: 121) |
| 14 | Rev | cccaaggggdatgctagttattgcgTCAATCCGTAGCCTCTTCATTCTCG |
| | | (SEQ ID NO: 122) |
| | Fwd | ataaatgcttcaataatattgaaaaaggaagagtATGGTAGCGATAAAAATGTATCCGGC |
| | | (SEQ ID NO: 123) |
| 15 | Fwd | gctaacttacattaattgcgttgcgcaACAATTTTTTGCCATAAGACGCTTTC |
| | | (SEQ ID NO: 124) |
| | Rev | ccaaggggdatgctagdattgcgCATTAGGACTAGTAGAAAAGTCTTGGG |
| | | (SEQ ID NO: 125) |
| 16 | Fwd | gctaacttacattaattgcgdgcgcaGGGATTTCCACCACCTCCC |
| | | (SEQ ID NO: 126) |
| | Rev | ccaaggggdatgctagdattgcgTGCATAGCCAATGAAGATAAACGTG |
| | | (SEQ ID NO: 127) |
| 17 | Fwd | gctaacttacattaattgcgdgcgcaGCGCAGCTGACAAAGATTGAC |
| | | (SEQ ID NO: 128) |
| | Rev | ccaaggggdatgctagdattgcgCGATAATAAAAAGGCTCCAATCCCTG |
| | | (SEQ ID NO: 129) |
| 18 | Fwd | gctaacttacattaattgcgdgcgcaACTAGCTAAGCAATAAGGGCG |
| | | (SEQ ID NO: 130) |
| | Rev | ccaaggggdatgctagttattgcgCAATCTCCGAGGTGGCCC |
| | | (SEQ ID NO: 131) |
| 19 | Fwd | gctaacttacattaattgcgdgcgcaTATTTTGCGTAGCTAGAACGCAATC |
| | | (SEQ ID NO: 132) |
| | Rev | ccaaggggdatgctagdattgcgTGGGTATTAGCTCATATCAGAACTAATACCC |
| | | (SEQ ID NO: 133) |
| 20 | Fwd | gctaacttacattaattgcgdgcgcaGTAAGACAAGGGTTGAGCAGGC |
| | | (SEQ ID NO: 134) |
| | Rev | ccaaggggdatgctagdattgcgCAATGGTGGGCTGATTAATTAGATGAG |
| | | (SEQ ID NO: 135) |
| 21 | Fwd | gctaacttacattaattgcgdgcgcaTAGCTATTGTGACTATGCTAACCATATG |
| | | (SEQ ID NO: 136) |
| | Rev | ccaaggggdatgctagdattgcgTTCAGTCTAAATACATACCTGTCGGG |
| | | (SEQ ID NO: 137) |
| 22 | Fwd | gctaacttacattaattgcgdgcgcaGTGCGCCTTATGTGATTACAACG |
| | | (SEQ ID NO: 138) |
| | Rev | ccaaggggdatgctagdattgcgCTCTCAGCCTAATGATTCCAGAATAG |
| | | (SEQ ID NO: 139) |
| 23 | Fwd | gctaacttacattaattgcgdgcgcaCGTGATGAATGAAGCGGCTAAATAC |
| | | (SEQ ID NO: 140) |
| | Rev | ccaaggggdatgctagdattgcgGTAAATCCTCGGGAAAACACAGG |
| | | (SEQ ID NO: 141) |
| 24 | Fwd | gctaacttacattaattgcgdgcgcaGATGGACTGGTACTGTAGATTCACC |
| | | (SEQ ID NO: 142) |
| | Rev | ccaaggggdatgctagdattgcgCAAAGACGCAGAGGCCATCAG |
| | | (SEQ ID NO: 143) |
| 25 | Fwd | gctaacttacattaattgcgdgcgcaGGGCTGTTTGGTTGAATTAAAAATACG |
| | | (SEQ ID NO: 144) |
| | Rev | ccaaggggdatgctagdattgcgCCTTGATTTAAAACTATCAGTAGTAGGAACG |
| | | (SEQ ID NO: 145) |
| 26 | Fwd | gctaacttacattaattgcgdgcgcaATAGAACGATGAAGGATGGAAGCTAC |
| | | (SEQ ID NO: 146) |
| | Rev | ccaaggggdatgctagttattgcgTTGTATTTTGTTGTGTATGGGCGG |
| | | (SEQ ID NO: 147) |
| 27 | Fwd | gctaacttacattaattgcgdgcgcaCGTGATTCAGTTCGCCAGAC |
| | | (SEQ ID NO: 148) |
| | Rev | ccaaggggdatgctagdattgcgCACTCGAAATGGATACCCTGAG |
| | | (SEQ ID NO: 149) |

| | |
|---|---|
| 28 | Synthesized |
| 29 | Synthesized |

TABLE 7
Predicted protein domains within validated defense systems. Transmembrane helices
were identified using TMHMM, and all other domains were identified using HHpred.

| | | | Representative | | | | |
|---|---|---|---|---|---|---|---|
| ID | Gene | Domain | HHpred Hit | Probability | Start | End | Residues |
| BREX | A | DUF1819 | PF08849.11 | 100 | 6 | 189 | 201 |
| type I | B | DUF1788 | PF08747.11 | 100 | 65 | 187 | 200 |
| | C | ATPase | PF07693.14 | 96.66 | 43 | 348 | 1213 |
| | C | DUF499 | PF04465.12 | 99.88 | 247 | 846 | 1213 |
| | D | Methyltransferase | PF02384.16 | 99.7 | 210 | 622 | 1201 |
| | E | PglZ | PF08665.12 | 99.12 | 474 | 650 | 865 |
| | F | Lon protease | PF13337.6 | 100 | 30 | 484 | 694 |
| | F | Lon protease | PF05362.13 | 99.9 | 486 | 693 | 694 |
| Druantia | A | DUF4338 | PF14236.6 | 99.92 | 45 | 339 | 404 |
| type I | B | CoiA | PF06054.11 | 99.77 | 1 | 182 | 548 |
| | C | Macoilin | PF09726.9 | 96.72 | 167 | 323 | 627 |
| | E | Helicase | PF00270.29 | 98.45 | 99 | 388 | 1836 |
| | E | Helicase | 5V9X_A | 97.55 | 1071 | 1208 | 1836 |
| | E | DUF1998 | PF09369.10 | 98.92 | 1626 | 1710 | 1836 |
| RT-Abi-P2 | A | RT | PF00078.27 | 99.09 | 68 | 291 | 515 |
| 1 | A | RT | PF00078.27 | 99.43 | 105 | 309 | 542 |
| | A | TIR | PF13676.6 | 97.91 | 411 | 536 | 542 |
| 2 | A | RT | PF00078.27 | 99.45 | 48 | 262 | 586 |
| | A | TOPRIM | cd01026 | 96.88 | 367 | 465 | 586 |
| 3 | A | Nuc_deoxy | PF15891.5 | 96.04 | 29 | 128 | 307 |
| | B | RT | PF00078.27 | 99.52 | 53 | 248 | 320 |
| 4 | A | RT | PF00078.27 | 99.63 | 54 | 328 | 425 |
| 5 | A | RT | PF00078.27 | 99.12 | 67 | 296 | 540 |
| 6 | A | RT | PF00078.27 | 99.14 | 59 | 263 | 494 |
| 7 | A | RT | PF00078.27 | 99.06 | 80 | 382 | 1232 |
| | A | Nitrilase | PF00795.22 | 98.89 | 953 | 1216 | 1232 |
| | B | Transmembrane | — | — | 4 | 26 | 144 |
| 8 | A | RT | PF00078.27 | 99.39 | 53 | 251 | 398 |
| | B | RT | PF00078.27 | 98.96 | 63 | 323 | 667 |
| 9 | A | ATPase | PF07693.14 | 99.6 | 33 | 364 | 851 |
| | B | Adenosine deaminase | PF00962.22 | 99.52 | 166 | 831 | 856 |
| 10 | A | ATPase | PF07693.14 | 97.62 | 39 | 390 | 1273 |
| | A | Transmembrane | — | — | 160 | 177 | 1273 |
| | A | Transmembrane | — | — | 199 | 218 | 1273 |
| 11 | A | ATPase | PF07693.14 | 99.8 | 15 | 385 | 643 |
| | C | QueC | PF06508.13 | 99.67 | 150 | 369 | 457 |
| | D | TatD DNase | PF01026.21 | 99.94 | 13 | 254 | 263 |
| 12 | A | DUF4011 | PF13195.6 | 99.81 | 33 | 308 | 1911 |
| | A | ATPase | PF13086.6 | 97.93 | 427 | 552 | 1911 |
| | A | Helicase | PF01443.18 | 97.82 | 1379 | 1636 | 1911 |
| | A | Endonuclease | PF18741.1 | 98.7 | 1683 | 1780 | 1911 |
| 13 | A | GHKL ATPase | 5V44_A | 99.46 | 1 | 241 | 2511 |
| | A | GHKL ATPase | 5V44_A | 99.03 | 1544 | 1756 | 2511 |
| | B | Helicase | 6BOG_B | 100 | 1 | 873 | 893 |
| 14 | A | MBL-fold hydrolase | PF00753.27 | 98.79 | 8 | 324 | 386 |
| | B | Protease | PF02122.15 | 98.23 | 2 | 187 | 1935 |
| | B | ATPase | PF14516.6 | 99.36 | 204 | 535 | 1935 |
| 15 | A | DUF4297 | PF14130.6 | 98.41 | 8 | 223 | 2092 |
| | A | ATPase | PF14516.6 | 99.44 | 250 | 597 | 2092 |
| 16 | A | ATPase | PF14516.6 | 98.93 | 316 | 643 | 1484 |
| 17 | A | Mrr | PF13156.6 | 97.05 | 17 | 162 | 1587 |
| | A | ATPase | PF14516.6 | 99.07 | 204 | 476 | 1587 |
| 18 | A | SIR2 | cd00296 | 99.26 | 22 | 244 | 769 |
| | A | ATPase | PF14516.6 | 97.6 | 312 | 464 | 769 |
| 19 | A | SIR2 | cd00296 | 99.44 | 21 | 253 | 1275 |
| | A | DUF4020 | PF13212.6 | 98.39 | 1114 | 1268 | 1275 |
| 20 | A | SIR2 | cd00296 | 99.47 | 21 | 240 | 1207 |
| 21 | A | SIR2 | cd00296 | 99.59 | 26 | 338 | 415 |
| | B | HerA helicase | 4D2I_B | 100 | 10 | 608 | 610 |
| 22 | A | DUF4297 | PF14130.6 | 99.05 | 1 | 191 | 394 |
| | B | HerA helicase | 4D2I_B | 100 | 7 | 568 | 571 |
| 23 | A | VWA | PF00092.28 | 98.93 | 14 | 203 | 277 |
| | B | Phosphatase | PF00481.21 | 99.74 | 5 | 232 | 239 |
| | C | Kinase | PF00069.25 | 100 | 34 | 296 | 561 |
| | C | ssDNA-binding | PF01336.25 | 96.18 | 344 | 435 | 561 |
| 24 | A | PHP | cd07436 | 99.36 | 4 | 238 | 891 |
| | A | ATPase | PF13166.6 | 99.74 | 266 | 836 | 891 |
| 25 | A | DUF1887 | PF09002.11 | 92.5 | 1105 | 1272 | 1272 |
| 26 | A | ATPase | PF13654.6 | 97.36 | 5 | 349 | 384 |
| | B | Protease | PF00082.22 | 99.87 | 264 | 561 | 754 |
| 27 | A | ATPase | PF07693.14 | 96.47 | 49 | 312 | 1022 |
| | A | DUF499 | PF04465.12 | 100 | 79 | 745 | 1022 |
| | B | DUF3780 | PF12635.7 | 100 | 1 | 187 | 195 |
| | C | DUF1156 | PF06634.12 | 99 | 18 | 81 | 945 |
| | C | Methyltransferase | PF01555.18 | 96.08 | 150 | 202 | 945 |
| | C | Methyltransferase | PF01555.18 | 97.76 | 548 | 682 | 945 |
| | D | PLD | cd09179 | 99.17 | 4 | 177 | 907 |
| | D | Helicase | 6BOG_B | 100 | 218 | 865 | 907 |
| 28 | A | RT | PF00078.27 | 99.35 | 136 | 351 | 613 |
| | B | DNA PolA | 2KFZ_A | 100 | 31 | 515 | 515 |
| 29 | A | RT | PF00078.27 | 99.37 | 34 | 241 | 311 |
| | B | ATPase | PF13175.6 | 99.8 | 64 | 432 | 550 |
| | C | HNH | PF01844.23 | 97.57 | 43 | 85 | 216 |

TABLE 8
Amino acid sequences of validated defense systems.

| # | Gene | Sequence |
|---|---|---|
| BREX | A | MIKNDKAWIGLLGGPLMSRESRVIAELLLTDPDEQTWQEQIVGHNILQASSPNTAKRYAATI |
| type I | | RLRLNTLDKSAWTLIAEGSERERQQLLFVALMLHSPVVKDFLAEVVNDLRRQFKEKLPGNSW |
| | | NEFVNSQVRHLPVLASYSDSSIAKMGNNLVKALAEAGYVDTPRRRNLQAVYLLPETQAVLQR |
| | | LGQQDLISILEGKR (SEQ ID NO: 150) |
| | B | MIDPVLEYRLSQIQSRINEDRFLKNNGSGNEIGFWIFDYPAQCELQVREHLKYLLRHLEKDH |
| | | KFACLNVFQIIIDMLNERGLFERVCQQEVKVGTETLKKQLAGPLNQKKIADFIAKKVDLAAQ |
| | | DFVILTGMGNAWPLVRGHELMSALQDVMGFTPLLMFYPGTYSGYNLSPLTDTGSQNYYRAFR |
| | | LVPDTGPAATLNPQ* (SEQ ID NO: 151) |
| | C | MNIEQIFEKPLKRNINGVVKAEQTDDASAYIELDEYVITRELENHRHFFESYVPATGEPRIR |
| | | MENKIGVWVSGFFGSGKSHFIKILSYLLSNRKVTHNGTERNAYSFFEDKIKDALFLADINKA |
| | | VHYPTEVILFNIDSRANVDDKEDAILKVFLKVFNERIGYCADFPHIAHLERELDKRGQYETF |
| | | KAAFADINGSRWEDERDAYYFISDDMAQALSQATQQSLESSRQWVEQLDKNFPLDINNFCQW |
| | | VKEWLDDNGKNILFMVDEVGQFIGKNTQMMLKLQTITENLGVICGGRAWVIVTSQADINAAI |
| | | GGMSSRDGQDFSKIQGRFSTRLQLSSSNTSEVIQKRLLVKTDEAKAALAKVWQEKADILRNQ |
| | | LAFDTTTTTALRPFTSEEEFVDNYPFVPWHYQILQKVFESIRTKGAAGKQLAMGERSQLEAF |
| | | QTAAQQISAQGLDSLVPFWRFYAAIESFLEPAVSRTITQACQNGILDEFDGNLLKTLFLIRY |
| | | VETLKSTLDNLVTLSIDRIDADKVELRRRVEKSLNTLERLMLIARVEDKYVFLTNEEKEIEN |
| | | EIRNVDVDFSAINKKLASIIFDDILKSRKYRYPANKQDFDISRFLNGHPLDGAVLNDLVVKI |
| | | LTPKDPTYSFYNSDATCRPYTSEGDGCILIRLPEEGRTWSDIDLVVQTEKFLKDNAGQRPEQ |
| | | ATLLSEKARENSNREKLLRVQLESLLAEADVWAIGERLPKKSSTPSNIVDEACRYVIENTFG |
| | | KLKMLRPFNGDISREIHALLTVENDTELDLGNLEESNPDAMREVETWISMNIEYNKPVYLRD |
| | | ILNHFARRPYGWPEDEVKLLVARLACKGKFSFSQQNNNVERKQAWELFNNSRRHSELRLHKV |
| | | RRHDEAQVRKAAQTMADIAQQPFNEREEPALVEHIRQVFEEWKQELNVFRAKAEGGNNPGKN |
| | | EIESGLRLLNAILNEKEDFALIEKVSSLKDELLDFSEDREDLVDFYRKQFATWQKLGAALNG |
| | | SFKSNRSALEKDAAAVKALGELESIWQMPEPYKHLNRITPLIEQVQVNHQLVEQHRQHALER |
| | | IDARIEESRQRLLEAHATSELQNSVLLPMQKARKRAEVSQSIPEILAEQQETKALQMDADKK |
| | | INLWIDELRKKQEAQLRAANEAKRAADSEQTYVVVEKTVIQPVPKKTHLVNVASEMRNATGG |
| | | EVLETTEQVEKALDTLRTTLLAVIKAGDRIRLQ* (SEQ ID NO: 152) |
| | D | MNTNNIKKYAPQARNDFRDAVQIKLTTLGIAADKKGNLQIAEAETIGETVRYGQFDYPLSTL |
| | | PRRERLVKRAREQGFEVLVEHCAYTWFNRLCAIRYMELHGYLEHGFRMLSHPETPTAFEVLD |
| | | HVPEVAEALLPENKAQLVEMKLSGNQDEALYRELLLGQCHALHHAMPFLFEAVDDEAELLLP |
| | | DNLTRTDSILRGLVDDIPEEDWEQVEVIGWLYQFYISEKKDAVIGKVVKSEDIPAATQLFTP |
| | | NWIVQYLVQNSVGRQWLQTYPDSPLKDKMEYYIEPAEQTPEVQAQLAAITPASIEPESIKVL |
| | | DPACGSDHILIEAYNVLKNIYEEGYRGRDIPQLILENNIFGLDIDDRAAQLSGFALLMMARQ |
| | | DDRRIFTRDVRLNIVSLQESLHLDIAKLWQQLNFHQQVQTGSMGDMFAENNALTQTDSAEYQ |
| | | LLMRTLKRFVNAKTLGSLIQVPQEEEAELKVFLDALYREQEGDFQQKTAAKAFIPFIQQAWI |
| | | LAQRYDAVVANPPYMGGNYMETELKNFVSSYYPQGKADLYSSFMVRLLLQLKDNRTLSLMTP |
| | | FTWMNLSSFEELRKIILTNFSIQSLVQPEYHSFFESAYVPICAFSISNTPLSWNAKFFDLSD |
| | | FYGEKNQAPNFQYAIKNDNKCHWKYNRITTDFLTPGYIIAYSLPDSALSCFKTSKKLHDVCN |
| | | LKQGLITGDNERYLRFSHESIYNSFSLNEKRKKTKWFPYQKGGAYRKWYGNNDYVVDWENDG |
| | | YSIKNFYNDKGKLRSRPQNIQFYCKEGLTWTSLTISSLSMRYVPNGYIFDAKGPMCPKSSLD |
| | | IWNILGYANSKVIDIFLKQLAPTMDYSQGPVGNVPFKFNDGDLNEIIKELVNIHKRDWDENE |
| | | TSFEFKRDMLVHFSRDINTIKGSFTLRQGENKKAINRTKFLEEMNNSFFINCFNLTDILSPE |
| | | IELNKITLTHATIEIDIQKIISYAIGCQMGRYSLDREGLVYAHEGNNGFADLVAEGAYKSFP |
| | | ADSDGILPLMDEEWFDDDVTSRVKEFIRTVWGEEYLRENLDFIAEVLKPKKGESALEITIRR |
| | | YLSTQFWKDHLKMYKKRPIYWLFSSGKEKAFECLVYLHRYNDATLSRMRTEYVVPLLARYQA |
| | | NIDRLNDQLDEASGGESTRLKRERDSLIKKFSELRSYDDRLRHYADMRISIDLDDGVKVNYG |
| | | KFGDLLADVKAITGNAPEVI* (SEQ ID NO: 153) |
| | E | MQNQDFIAGLKAKFAEHRIVFWHDPDKRFIEELEQLKLESVTLINMTHESQLAVKKRIEIDE |
| | | PEQQFLLWFPHDAPPHEQDWLLDIRLYSSEFHADFAAITLNTLGIPQLGLREHIQRRKAFFS |
| | | TKRTQALKNLATEQEDEASLDKKMIAVIAGAKTAKTEDILFNLITYQYVNQQIEDDSELENT |
| | | QAMLKRHGLDSVLWEMLNHEMGYQAEEPSLENLLLKLFCTDLSAQADPQQRAWLEKNVLLTP |
| | | SGRASALAFMVTWRADRRYKEAYDYCAQQMQAALHPEDHYRLSSPYDLHECETTLSIEQTII |
| | | HALVTQLLEESTTLDREAFKKLLSERQSKYWCQTQPEYYAIYDALRQAERLLNLRNRHIDGF |
| | | HYQDSATFWKAYCEELFRFDQAYRLFNEYALLVHSKGAMILKSLDDYIEALYSNWYLAELSR |
| | | NWNEVLEAENEMQAWQIPGVPRQQNFFNEVVKPQFQNPQIKRVFVIISDALRYEVAEELGNQ |
| | | INTEKRFTAELRSQLGVLPSYTQLGMAALLPHEQLCYQPGNGDIVYADGLSTSGIPNRDTIL |
| | | KNYKGMAIKSKDLLELKNQEGRDLIRDYEVVYIWHNTIDATGDTASTEDKTFEACRTAVAEL |
| | | KDLVTKVINRLHGTRIFVTADHGFLFQQQALSVQDKTTLQIKPENTIKNHKRFIIGHQLPAD |
| | | DFCWKGKVADTAGVSDNSEFLIPKGQIRFFSGGARFVHGGTMLQEVCVPVLQIKALQKTAAE |
| | | KQPQRRPVDIVAYHPMIKLVNNIDKVSLLQTHPVGELYERPRILNIYIVDNANNVVSGKERI |
| | | SFDSDNNTMEKRVREVTLKLIGANFNRRNEYWLILEDAQTETGYQKYPVIIDLAFQDDFF* |
| | | (SEQ ID NO: 154) |
| | F | MQTHHDLPVSGVSAGEIASEGYDLDALLNQHFAGRVVRKDLTKQLKEGANVPVYVLEYLLGM |
| | | YCASDDDDVVEVQGLQNVKRILADNYVRPDEAEKVKSLIRERGSYKIIDKVSVKLNQKKDVY |
| | | EAQLSNLGIKDALVPSQMVKDNEKLLTGGIWCMITVNYFFEEGQKTSPFSLMTLKPIQMPNM |
| | | DMEEVFDARKHFNRDQWIDVLLRSVGMEPANIEQRTKWHLITRMIPFVENNYNVCELGPRGT |
| | | GKSHVYKECSPNSLLVSGGQTTVANLFYNMASRQIGLVGMWDVVAFDEVAGITFKDKDGVQI |
| | | MKDYMASGSFSRGRDSIEGKASMVFVGNINQSVETLVKTSHLLAPFPTAMIDTAFFDRFHAY |
| | | IPGWEIPKMRPEFFTNRYGLITDYLAEYMREMRKRSFSDAIDKFFKLGNNLNQRDVIAVRRT |
| | | VSGLLKLMHPDGAYSKEDVRVCLTYAMEVRRRVKEQLKKLGGLEFFDVNFSYIDNETLEEFF |
| | | VSVPEQGGSELIPAGMPKPGVVHLVTQAESGMTGLYRFETQMTAGNGKHSVSGLGSNTSAKE |
| | | AIRVGFDYFKGNLNRVSAAAKFSDHEYHLHVVELHNTGPSTATSLAALIALCSILLAKPVQE |
| | | MQMVVLGSMTLGGVINPVQDLAASLQLAFDSGAKRVLLPMSSAMDIPTVPAELFTKFQVSFY |
| | | SDPVDAVYKALGVN* (SEQ ID NO: 155) |
| Druantia | A | MHKYPSIIVNINLREAKLKKKVREHLQSLGFTRSDSGALQAPGNTKDVIRALHSSQRAERIF |
| type I | | ANQKFITLRAAKLIKFFASGNEVIPDKISPVLERVKSGTWQGDLFRLAALTWSVPVSSGFGR |
| | | RLRYLVWDESNGKLIGLIAIGDPVFNLAVRDNLIGWDTHARSSRLVNLMDAYVLGALPPYNA |
| | | LLGGKLIACLLRSRDLYDDFAKVYGDTVGVISQKKKQARLLAITTTSSMGRSSVYNRLKLDG |
| | | IQYLKSIGYTGGWGHFHIPDSLFIELRDYLRDMDHAYADHYMFGNGPNWRLRTTKAALNALG |
| | | FRDNLMKHGIQREVFISQLAENATSILQTGKGEPDLTSLLSAKEIAECAMARWMVPRSIRNP |
| | | EYRLWKARDLFDFISNDSLNFPPFDEIAKTVV* (SEQ ID NO: 156) |
| | B | MNYAIDKFTGTLELAARATKYAQYVCPVCKKGVNLRKGKVIPPYFAHLPGHGTSDCENFVPG |
| | | NSIIVETIKTISKRYMDLRLLIPVGSNSREWSLELVLPTCNLCRAKITLDVGGRSQTLDMRS |
| | | MVKSRQIGAELSVKSYRIVSYSGEPDPKFVTEVERECPGLPSEGAAVFTALGRGASKGFPRA |
| | | QELRCTETFAFLWRHPVAPDFPDELEIKSLASKQGWNLALVTIPEVPSVESISWLKSFTYLP |
| | | VVPARTSITAIWPFLNQKTSINHVECVYSDTILLSTNMAPTSSENVGPTMYAQGSSLLLSAV |
| | | GVETSPAFFILNPGENDFVGVSGSIEQDVNLFFSFYKKNVSVPRKYPSIDLVFTKRNKEKTI |
| | | VSLHQRRCIEVMMEARMFGHKLEYMSMPSGVEGVARIQRQTESNVIKLVSNDDIAAHDKSMR |
| | | LLSPVALSQLSDCLANLTCHVEIDFLGLGKIFLPGSSMLSLDDGKFIELSPNLRSRILSFIL |
| | | QMGHTLHGFSLNNDFLLVEKLVDLQPEPHLLPHYRALVKEVKTNGFECNRFR* |
| | | (SEQ ID NO: 157) |
| | C | MSYQYSQEAKERISKLGQSEIVNFINEISPTLRRKAFGCLPKVPGFRAGHPTEIKEKQKRLI |
| | | GYMFQSHPSSEERKAWKSFSLFWQFWAEEKIDKSFSMIDNLGLKENSGSIFIRELAKNFPKV |
| | | ARENIERLFIFSGFADDPDVINAFNLFPPAVVLARDIVIDTLPRILDELEARISLIADNVEK |
| | | KNNHIKELELKIDAFSEQFDNYFNNEKSSLKIINELQSLINSETKQSDIANKAIDELYHFNE |
| | | KNKQLILSLQEKLDFNALAMNDISEHEKLIKSMANDISEFKNALTILCDNKIKNNELDYVNE |
| | | LKKLTERIDTLEINTSQASEVSVTNRFTKFHEIAHYENYEYLSSSEDISNRISLNLQAVGLT |
| | | KNSAEKLARLTLATFVSGQIIQFSGSLADIIADAIAIAGAPRYHIWRVPVGIISDMDAFDFI |
| | | ETIAESSRCLLLKGANLSAFEIYGAAIRDIVVQRQIHPTNYDHLALIATWKQGPATFPDGGM |
| | | LAELGPVIDTDTLKMRGLSATLPQLKPGCLAKDKWTNIDGLHLDSVDDYVDELRALLDEAGF |
| | | DGGTLWKRMIHIFYTSLIRIPNGNYIYDLYSVLSFYTLTWAKIKGGPVQKIEDIANRELKNY |
| | | SAKISS*(SEQ ID NO: 158) |
| | D | MEWRAVSRDKALDMLSTALNCRFDDEGLRISAVSECLRSVLYQYSISETEEARQTVTSLRLT |
| | | SAVRRKLVPLWPDIADIDNAIHPGIMSILNSLAELGDMIKLEGGNWLTAPPHAVRIDNKMAV |
| | | FFGGEPSCTFSTGVVAKSAGRVRLVEEKVCTGSVEIWDANEWIGAPAEGNEEWSSRLLSGTI |
| | | SGFIDAPGNMSETTAYVRGKWLHLSELSFNKKQIYLCRMSVDNHFSYYLGEIEAGRLCRMNS |
| | | LESSDDVRRLRFFLDTKCNCPLKVRIKISNGLARLRLTRRLPRRETKVLLLGWRESGFENEH |
| | | SGITHHVFPEEILPIVRSAFEGLGIIWINEFTRRNEI* (SEQ ID NO: 159) |
| | E | MINKNKVTERSGIHDTVKSLSENLRKYIEAQYHIRDEGLIAERRALLQQNETIAQAPYIEAT |
| | | PIYEPGAPYSELPIPEAASNVLTQLSELGIGLYQRPYKHQSQALESFLGENASDLVIATGTG |
| | | SGKTESFLMPIIGKLAIESSERPKSASLPGCRAILLYPMNALVNDQLARIRRLFGDSEASKI |
| | | LRSGRCAPVRFGAYTGRTPYPGRRSSRRDELFIKPLFDEYNKLANNAPVRAELNRIGRWPSK |
| | | DLDAFYGQSASQAKTVYSGKKTGKQFVLNNWGERLITQPEDRELMTRHEIQNRCPELLITNY |
| | | SMLEYMLMRPEIRNIFEQTKEWLKADEMNELILVLDEAHMYRGAGGAEVALLIRRLCARLDI |
| | | PRERMRCILTSASLGSIEDGERFAQDLTGLSPTSSRKFRIIEGTRESRPESQIVTSKEANAL |
| | | AEFDLNSFQCVAEDLESAYAAIESLAERMGWQKPMIKDHSTLRNWLFDNLTGFGPIEITLIE |
| | | IVSGKAVKLNILSENLFPDSPQQIAERATDALLALGCYAQRADGRVLIPTRMHLFYRGLPGL |
| | | YACIDPDCNQRLGNHSGPTILGRLYTKPLDQCKCASKGRVYELFTHRDCGAAFIRGYVSSEM |
| | | DFWHQPNGPLSEDEDIDLVPIDILVEETPHVHSDYQDRWLHIATGRLSKQCQDEDSGYRKVF |
| | | IPDRVKSGSEITFDECPVCMRKTRSAQNEPSKIMDHVTKGEAPFTTLWTQISHQPASRPIDG |
| | | KHPNGGKKVLIFSDGRQKAARLARDIPRDIELDLFRQSIALACSKLKDINREPKPTSVLYLA |
| | | FLSVLSEHDLLIFDGEDSRKVVMARDEFYRDYNSDLAQAFDDSFSPQESPSRYKIALLKLLC |
| | | SNYYSLSGTTVGFVEPSQLKSKKMWEDVQSKKLNIESKDVHALAVAWIDTLLTEFAFDESID |
| | | STLRIKAAGFYKPTWGSQGRFGKALRKTLIQYPAMGELYVEVLEEIFRTHLTLGKDGVYFLA |
| | | PNALRLKIDLLHVWKQCNDCTALMPFALEHSTCLACGSNSVKTVEPSESSYINARKGFWRSP |
| | | VEEVLVSNSRLLNLSVEEHTAQLSHRDRASVHATTELYELRFQDVLINDNDKPIDVLSCTTT |
| | | MEVGVDIGSLVAVALRNVPPQRENYQQRAGRAGRRGASVSTVVTYSQNGPHDSYYFLNPERI |
| | | VAGSPRTPEVKVNNPKTARRHVHSFLVQTFFHELMEQGIYNPAEKTAILEKALGTTRDFFHG |
| | | AKDTGLNLDSFNNWVKNRILSTNGDLRTSVAAWLPPVLETGGLSASDWFAKVAEEFLNTLHG |
| | | LAEIVPQTAVLVDEENEDDEQTSGGMKFAQEELLEFLFYHGLLPSYAFPTSLCSFLVEKIVK |
| | | NIRGSFEVRTVQQPQQSISQALSEYAPGRLIVIDRKTYRSGGVFSNALKGELNRARKLFNNP |
| | | KKFIHCDKCSFVRDPHNNQNSENTCPICGGILKVEIMIQPEWGPENAKELNEDDREQEITYV |
| | | TAAQYPQPVDPEDFKFNNGGAHIVFTHAIDQKLVTVNRGKNEGESSGFSVCCECGAASVYDS |
| | | YSPAKGAHERPYKYIATKETPRLCSGEYKRVFLGHDFRTDLLLLRITVGSPLVTDTSNAIVL |
| | | RMYEDALYTIAEALRLAASRHKQLDLDPAEFGSGFRILPTIEEDTQALDLFLYDTLSGGAGY |
| | | AEVAAANLDDILTATLALLESCECDTSCTDCLNHFHNQHIQSRLDRKLGASLLRYALYGMVP |
| | | RCASPDIQVEKLSQLRASLELDGFQCIIKGTQEAPMIVSLNDRSIAVGSYPGLIDRPDFQHD |
| | | VYKSKHTNAHIAFNEYLLRSNLPQSHQNIRKMLR* (SEQ ID NO: 160) |
| RT-Abi-P2 | A | MKKVYELTSEEALSYFLRHDSYTTLELPAYINFTTLLNDINSSIHNKKIKIEPTAKELMGKD |
| | | INYEVLVSKDGLYSWRRITLINPLYYVYFCRKITAPATWEIITEKFKSFESNDLFTCSSIPV |
| | | RKDNSSNIAASVMNWWEDFEQKSLALALEYEFWSTDISNFYPSIYTHSFEWVFISKEEAKKK |
| | | KSKNNPGGLIDSHIQMMMNNQTNGIPLGSTLMDTFAELILGQIDIELRKKTNELKIINYKWR |
| | | YRDDYRIFSNSKDDLDIISKCLVNVLGDFGLDLNSKKTELYEDIILHSLKQAKKDYIKEKRH |
| | | KSLQKMLYSIYLFSLKHPNSKTTVRYLNDFLRNLFKRKTIKDNGQQVDAMLGIISSIMAKNP |
| | | TTYPVGTAIFSKLLSFLYGDDTQKKLTKLEQLHKKLDKQPNTEMLDIWFQRTQAKINLEWNK |
| | | SYKSALCVRINDELTKEKTFSVNNLWNIDWIQGKETSPNKAKILSLLRKTKIVDTDKFDKMD |
| | | DNITPEEVNLFFKEHSN* (SEQ ID NO: 161) |
| 1 | A | MSLHDKLLMHNFALANKKSPDFISELPQIEPKPYSNGHKIKWINHTLTSTEVTPPDNLIKIC |
| | | ILIESGEIAITSVSDIANLLGVPAGQLLYILYRKKDNYRTFEIEKKNGKKRVINAPCGGLSI |
| | | LQTRLKPVLEYFYRPKKSAHGFDCGKSIITNAGMHIKKNFWNIDLENYFESISFARVYGIFK |
| | | SKPFNFAHPAATVLAQLCTHNGKLPQGACTSPILAMASASLDKQLTQFAGRKKISYSRYADD |
| | | ITFSFNQRNIDIIKKNDDGSYSLSETIDNIISKNGFKINYDKFRVQTRNTRQSVTGLWNDKV |
| | | NINRRYIRITRSMIHRWTDDKLKYALLFATEKGYQAKDNNHAIQIFRNHIYGRLSFIKMVRG |
| | | KDYPGYLKLMSYMSHNDPLKTQEGLRAMKETENFDVFICHASEDKKDIAIPIYDELTKLKIS |
| | | AFIDHVEIKWGDSLIDKINAALVKSKYVIAILSANSVNKEWPQKELRAVLASEISSGDVKLL |
| | | TLLKKEDEEVVNLSLPLLSDKFYMVYDNNPEVVANNIKSLLQR* (SEQ ID NO: 162) |
| 2 | A | MTKTSKLDALRAATSREDLAKILDVKLVFLTNVLYRIGSDNQYTQFTIPKKGKGVRTISAPT |
| | | DRLKDIQRRICDLLSDCRDEIFAIRKISNNYSFGFERGKSIILNAYKHRGKQIILNIDLKDF |
| | | FESFNFGRVRGYFLSNQDFLLNPVVATTLAKAACYNGTLPQGSPCSPIISNLICNIMDMRLA |
| | | KLAKKYGCTYSRYADDITISTNKNTFPLEMATVQPEGVVLGKVLVKEIENSGFEINDSKTRL |
| | | TYKTSRQEVTGLTVNRIVNIDRCYYKKTRALAHALYRTGEYKVPDENGVLVSGGLDKLEGMF |
| | | GFIDQVDKFNNIKKKLNKQPDRYVLTNATLHGFKLKLNAREKAYSKFIYYKFFHGNTCPTII |
| | | TEGKTDRIYLKAALHSLETSYPELFREKTDSKKKEINLNIFKSNEKTKYFLDLSGGTADLKK |
| | | FVERYKNNYASYYGSVPKQPVIMVLDNDTGPSDLLNFLRNKVKSCPDDVTEMRKMKYIHWYN |
| | | LYIVLTPLSPSGEQTSMEDLFPKDILDIKIDGKKFNKNNDGDSKTEYGKHIFSMRVVVDKKR |
| | | KIDFKAFCCIFDAIKDIKEHYKLMLNS* (SEQ ID NO: 163) |
| 3 | A | MNKKFTDEQQQQLIGHLTKKGFYRGANIKITIFLCGGDVANHQSWRHQLSQFLAKFSDVDIF |
| | | YPEDLFDDLLAGQGQHSLLSLENILAEAVDVIILFPESPGSFTELGAFSNNENLRRKLICIQ |
| | | DAKFKSKRSFINYGPVRLLRKFNSKSVLRCSSNELKEMCDSSIDVARKLRLYKKLMASIKKV |
| | | RKENKVSKDIGNILYAERFLLPCIYLLDSVNYRTLCELAFKAIKQDDVLSKIIVRSVVSRLI |
| | | NERKILQMTDGYQVTALGASYVRSVFDRKTLDRLRLEIMNFENRRKSTFNYDKIPYAHP* |
| | | (SEQ ID NO: 164) |
| | B | MKSAEYLNTFRLRNLGLPVMNNLHDMSKATRISVETLRLLTYTADFRYRIYTVEKKGPEKRM |
| | | RTIYQPSRELKALQGWVLRNILDKLSSSPFSIGFEKHQSILNNATPHIGANFILNIDLEDFF |
| | | PSLTANKVFGVFHSLGYNRLISSVLTKICCYKNLLPQGAPSSPKLANLICSKLDYRIQGYAG |
| | | SRGLIYTRYADDLTLSAQSMKKVVKARDFLFSHPSEGLVINSKKTCISGPRSQRKVTGLVIS |
| | | QEKVGIGREKYKEIRAKIHHIFCGKSSEIEHVRGWLSFILSVDSKSHRRLITYISKLEKKYG |
| | | KNPLNKAKT* (SEQ ID NO: 165) |
| 4 | A | MNNDDYPWFRKRGYLHFDEPVSLKKAVKYVSSPEKIIKHSFLPFLSFEVKSFKIKKDKSTKQ |
| | | LSKTEKLRPIAYSSHLDSHIYAFYAEYLTGHYELLIQENNLHENILAFRSLNKSNIEFAKRA |
| | | FDTITEMGECSAVALDLSGFFDNLDHQILKHQWCKVIGTEALPQDHFAIYKSITRYSKVDKN |
| | | RAYEILGISKNNPKYNRRKICTPVDFRNKIRKNGLITVNNSQKGIPQGSPTSALLSNIYMLD |
| | | FDTEMRDYAQERGGHYYRYCDDMLFIVPTKYNKTLAGDVAQRIKHLKVELNTKKTEIRDFIY |
| | | KDSTLVANMPLQYLGFIFDGSNILLRSSSLARYSERMKRGVRLAKATMDSKNRIRENKGEAL |
| | | KALFKKKLYARYSHIGRRNFLTYGYRAAKIMNSKAIKRQLKPLQKRLENEILK* |
| | | (SEQ ID NO: 166) |
| 5 | A | MVIFDEKRHLYEALLRHNYFPNQKGSISEIPPCFSSRTFTPEIAELISSDTSGRRSLQGYDC |
| | | VEYYATRYNNFPRTLSIIHPKAYSKLAKHIHDNWEEIRFIKENENSMIKPDMHADGRIIIMN |
| | | YEDAETKTIRELNDGFGRRFKVNADISGCFTNIYSHSTPWAVIGVNNAKIALNTKVKNQDKH |
| | | WSDKLDYFQRQAKRNETHGVPIGPATSSIVCEIILSAVDKRLRDDGFLFRRYEDDYTCYCKT |
| | | HDDAKEFLHLLGMELSKYKLSLNLHKTKITNLPGTLNDNWVSLLNVNSPTKKRFTDQDLNKL |
| | | SSSEVINFLDYAVQLNTQVGGGSILKYAISLVINNLDEYTITQVYDYLLNLSWHYPMLIPYL |
| | | GVLIEHVYLDDGDEYKNKFNEILSMCAENKCSDGMAWTLYFCIKNNIDIDDDVIEKIICFGD |
| | | CLSLCLLDSSDIYEEKINNFVSDIIKLDYEYDIDRYWLLFYQRFFKDKAPSPYNDKCFDTMK |
| | | GYGVDFMPDENYKTKAESYCHVVNNPFLEDGDEIVSFNDYMAIA* (SEQ ID NO: 167) |
| 6 | A | MTSTIDFYESDFSATLYPLKTNQILLKHHSQEMSEYIYQKVINPAYPTDSFLSQQKVFSTKP |
| | | KGHLRRTVKLDPVAEYFTYDVTYRNRKIFRPEVSESRKSFGYIFRNGSRIPIHVSYNEYKQS |
| | | LKKYSELYSHSIHFDIASYFNSLYHHDIIHWFSSKEGVSPADVEALGQFFREINSGRSIDFM |
| | | PQGIYPAKMIGNEFLKFVDLHGRLKSAQIVRFMDDFTIFDNDIETLNNDFIRIQQLLGQVSL |
| | | NINPSKTTFDNVMGDVNETLTQIKSSLKEIITEYEHIPTASGVEWETNIEIIKHLDDEQVNK |
| | | LIDLLKDEKIEESDADLILGFLRTHNDSLLSQMPMLLGRFPNLIKHIYTICSGITDKSGLVK |
| | | ILLSYLNTNNNFLEYQLFWIGAIVEDYLLGVGEYGSVLHKLYELSGDFKIARAKVLEIPEQG |
| | | FGFKEIRNEYLRTGQSDWLSWSSAIGTRNLKSAERNYILDYFSKGSPINYLVASCVKKL* |
| | | (SEQ ID NO: 168) |
| 7 | A | MKLLDKKYYNLEPKYEYLKDSFILGLAWKKTDSFVRTHNWYADILELDKCAFDISDEVTNWS |
| | | NEISKNALSKSDIELIPAPKGASWFINQGKWTTNKDNRKIRPLANISIRDQSFATAVTMCLA |
| | | DAIETRQKDCSLSNLGYAEHVKNKVVSYGNRLVCDWDNERARFRWGGSEYYRKFSSDYRSFL |
| | | QRPIYIGRETVNKVSGIDDVYIISLDLKNFFGSIKINLLLEKIKKISADHYAAKFINDNEFW |
| | | TLANRILSWDWPEESLSLLESLDKEKNVGLPQGLASAGALANAYLIEFDESLISKLRTKIED |
| | | SQIILHDYCRYVDDIRLVISGEALESNKIKESIHALVQGILDETLAQNPSDNEPYLKINDSK |
| | | TYILELSDIDNGSGLTNRINEIQHEVGASSIPERNGLDNNIPALQQLLLTEQDNFSEDVDSL |
| | | FPGFKNDKSIKVESVRRFSAHRLEKSLAKKSKLISPEERKQFDNETSLIAKKLLKAWLKDPS |
| | | IMVIFRKAIAINPNLDAYSTILEIIFSRIQRNRDKRDKYIMLYLLSDIFRSVIDVYRNLESE |
| | | YVDDYQKLMGEVTLFAQKILSCKSFIPNYAYQQALFYLAVINKPFIASNKASFDLARLQCVL |
| | | IKQHLEPLNSSDGYLFEVSAQISKDYRANAAFLLSHTNSNKVVDLIIEKFAFRGGEFWNAIW |
| | | KEIVRMQDKDRINEFRWAISKYESKPNSSEHYLSSVISFKENPFRYEHALLKLGVALVELFD |
| | | DTEKNVWQPDGKQYSPHEIKVKLEGNSTSWGELWRPNFSISCSIDKKGEPGKDPRYISPEWL ANYPQTQNDEQKIYWVCSVLRSAALGNVDYTQRNDLKLDKAKYDGIHSQFYKRRMGMLHTPE |
| | | SIVGSYGTITDWFASFLQHGLQWPGFSSSYISQEDILSITNIIEFKNCLLERLGYLNKQICI |
| | | SSNVPTLPTVVNRPELASNHFRIVTVQQLFPKDTNFHPSDVTLANPDVRWKHREHLAEICKL |
| | | TEQTLNAKLKTESREHTSTADLIVFSELAVHPEDEDIVRALAFRTKAIIFSGFVFCEQDGRI |
| | | VNKARWIIPDSSESGTQWRVRDQGKHHMTSDEVALGIQGYRPSQHIISIEGFIPEGPFKLTG |
| | | AICYDATDIKLAADLRDLTDMFVIAAYNKDVDTFDNMASALQWHMYQHIVITNTGEYGGSTM |
| | | QAPYKEKYHKLISHAHGTGQIAISTADIDLAAFRRKLQTYKKTKTQPAGYNRKH* |
| | | (SEQ ID NO: 169) |
| | B | MDTLVKLATIISPLISAGVAIWAILVAKKTISESKEIAKKTIADTAYQAYLQLAMENPQFSK |
| | | GYSADCRQERDPMYDQYVWYVARMIFCFEKIIEVEVNLKDSSWANTLEKHLKFHSEHFKKTN |
| | | VVEEALYIPPILDLIRCAAN* (SEQ ID NO: 170) |
| 8 | A | MLNQSFSVSNLIKLLKKTDPKRYKTGRNSAEYKKYIADKVNGSIETYSFGSISNSRTNNKNV |
| | | YIFKDFMDVLVARKINDNIKRVYSVKQNNRHDIIKKVNTVLSEPVNYYIYRLDIKSFYESID |
| | | KNIVFQRINNNPIISHNTKKFINGLFKHNAFSANNGLPRGMGLSATLSEIFMEEFDAELARL |
| | | PEVFYASRYVDDIIVFSFYKIPDYKNYFSRILPNGLHLNERKCSEYTIEDTSTKHSEIEFLG |
| | | YSFIIHHGLKNQRRHVVIRISEEKIKKIKRRIALAVKDYSMNSDAELLKKRIKYLTGNTLVN |
| | | SNSNKTDALYSGIYYNYQHLTDKTQLKELDIFKNRMLFSSKGEVGRKILAAGHNLLTAPKKY |
| | | SFLAGFEKRLLSSFKREDIIKINKVW* (SEQ ID NO: 171) |
| | B | MKIKISKSDYKRVLLTDILPYEVPILFSNEGFYKLISENKVLPGTFSEGLKLDSYTIPYSYK |
| | | IKKGLASSRSLGIIHPSTQLRICDFYDKYEHLMVHMCTKSPFSLRYPSKIGSYYYEKDFLKS |
| | | RINLKDGLVQFHNHGFDSQETSSSSHFSYKKYPFIYKFYESYEFHRLERKFRKLLKLDIAKC |
| | | FSHIYTHSVSWAVKSKEFSKVNRTYNSFEGCLDKLFQDANYGETNGIIIGPEFSRIFAEIIL |
| | | QRVDLNVESHLNLEPGIVKDKSYATRRYVDDYFIFADDDETFKLTEFVLANELEKYKLYLNE |
| | | SKKEFIERPFVTGATMAKNDIAEIIEDLYGSLIHTEKLDELTAMVNLNPDVKIQPENMNDLF |
| | | PLKGVWNKKLHADKFIKRIKIAVRKNNTTFDLVSSYLLSAIKSKFFKVIRLLRMFDLSGKED |
| | | ITYKFFSIFNEVIFFIYAMDFRVRQTYIISQVILEINSFANKQASDISEVIKKNTFDELLMC |
| | | MKSMGNIHERPVELSNLLICMKGLGEQYKLNPDEFKDLLGISENECFYDLEYFSTCSMLHYI |
| | | GDDVLYLKMKEDIVLAIQSLISGRNDIKKDTETFMLFLDMMTCPYLTVKHKRIIYRTYVEAN |
| | | TGQKRFTNAVIDSEIDSLKNNVIFFNWSGDADLEHVLYKKELRTAYE* |
| | | (SEQ ID NO: 172) |
| 9 | A | MTSEIVLNLDFPEYKDDFCTDSIDEQDNELWQQQANKKLLSFLEVMGEEARRYKENNSRSTH |
| | | PHYKTLSSYHHAIFISGARGAGKTVFMRNARFSWQKHYNKDLKRPKLYFIDVTDPTLLNIDD |
| | | RFSEVIIASIYATVEKRMKQPDIAQNIKDNFINSLKTLSGALGKSKDYDEYRGIDRIQKYRS |
| | | GIHLEKYFHQFLISSVELLDCDALVLPIDDVDMKIDNAFGVLDDIRCLLSCPLVLPLVSGDN |
| | | DLYRFIAKSKFEELLNRKANSNYAKEGSEIAERLSEAYITKVFPSHVKIPLQPIDELLPYLY |
| | | IHSNEDENKQHTSYSEFIKLVQQKFYFLCNGQERSTNWPQPRSAREVTQLIRSLPPSTLSKE |
| | | DDSGTDLWQRFAVWAEERRDGLALTNVESYLFIKNAKAVEDLNLSNLIAFNPLLQKGKYPWA |
| | | EKDFYKQQSQRRKELNAPETNSGILNTVFSEQRKDFILRSMPALELIMEPMYVTKTVAEKND |
| | | NSALIAIYTHSDYYSQQQNRRCHIFFGRAFEIMFWSVLAKTENLPQEFYEKDKFKSLFGNIF |
| | | KKVPFYSIFSMNPTKVVDEENDDGSEPDFSQKLDDSINELVEDIYIWATSNKLRAFKNKNLI |
| | | PLMTCVFNKVFSMNVLRKNVQDRVKFRDEHLSDLAKRFEYMFINAEFTFIREGVVVNTNVAT |
| | | GAAPARVRNLSEFNRYDKTLSRNMSGILSVKEDNGLTIVKESEGDIADLLFEIWHSPLFKLT |
| | | TRTCYPIGKINSQNTAQENLSSDFNSFFENGINFELIKQYYWQTSNHDNIRTADVREWATSR |
| | | LNEAIILFSWMKESKSIKAKIDGQSYEGRLFRGLQQALEGYEEV* (SEQ ID NO: 173) |
| | B | MFNQDPYWLIPTLCLASDRIFYAQLRDHLGQKSSGERKKEKNGYILVQAAQDYQFYFGGRIR |
| | | KEDVQNNALMWQIETGNENCLSMLDSLSAYFLTWRGNCFEVRRERLEPWLMICSVIDPAWII |
| | | AYAYQQLIKQNVVCDSELISLLTEHQCPFAFPKGRGDISFADNHVHLNGHGYSSISMLNFID |
| | | GNYKVKKGIKWPYRQEYTLFESGLLDKNDLPRWLSAYSSCLLKNVYNSFQQGKRSEVDFTCL |
| | | KDAVETVLADEDKYYFLEVASLYDVVTLQQRVLYEAAQQKYHSHQRWLLYTCGIMLGTESED |
| | | YANALANLIRISNILRNYMVVSAVGLGQFIDFFGFNYRRITKPADTNNRVHYDSSAGISREY |
| | | RVSPDFVLGSGVMPDIYARQLFDFYCTQARKGVPEQGHIVVHFTRSFPDKKSTYDKLLTECR |
| | | ERLRSQCDYFGRFLTSLTLQSIEYKNLSTDEDRSIDIRKLVRGYDVAGNENELQIEVFAPVL |
| | | RVLRAAKFKGEGVNFKRLQRPFITVHAGEDYCHILSGLRAMDEAVEFCMLGEGDRIGHGLAL |
| | | GVDIKLWANRQKRAYLTVGQHLDNLVWAYHQAVLLSQHIVEHIPVMHELRDKIHYWSHQLYS |
| | | ETYTPDLLFKAWLLRRNWPDYKSIISDPANINEWVPDQHILVSTDETTAKARKIWERYLNSG |
| | | LAENDVFNRIISVNCAPDTAQNFSMTFNENEDILSKGELLLYEAIQDFLIEKYSRLGLVIEA |
| | | CPTSNIYIGRLEKYHEHPLFRWNPPDSQWIKPGGKFNRFGLRTGPLSVCINTDDSALMPTTI |
| | | ENEHRLMRDCAIHFYGIGTWMADLWINSIRIKGIEIFKGNHLSQDLDNLI* |
| | | (SEQ ID NO: 174) |
| 10 | A | MIMSTPWLTPIVADSDHAEANAVSYEALTPTELDSDKAGCYISALNYAYEHPDIRNIAVTGP |
| | | YGAGKSSVLKTWCKAHNGTLRVLTVSLADFDMQRHVDESNGDSSSDEGTKNTGSVEKSEYSE |
| | | LQQILYKNKKHELPCSRIDRISDVTAGQILRSASFLTGTILLSGAALFFLAPDYVTTKLSLP |
| | | GAFARYLLECPFGVRVSGAVASVMGSLCLLLNQLHRIGIFDRKVSLDKVDLLKGAVTTRASS |
| | | PSLLNVYIDEIVYFFDSTKYDWIFEDLDRFNNGRIFVKLREINQIINNCLSDRKPVKFIYAV |
| | | RDGIFNSAESRTKFFDFVMPVIPVMDNQNAYEHFVKKFKEEEINNNLSECISRIATFIPNMR |
| | | VMHNITNEFRLYQNLVNSRENLAKLLAMIAYKNLCAEDYHGIDSKKGVLYHFIQSYLDHEIQ |
| | | NELLHSANNELEDMAQSLVAITNEKLANRENLREELLMPYLSKNYSGALVFYTEGRQISLDD |
| | | LIQDEDEFLMLLDKENIQVVTPYNRQNFLMINQRDTEKLKQQYEKRCHLIETKSVDNITRVK |
| | | NNISSLESLRTEILSGTVADIAEKMTNEGFVAWIKKKEDTGVLTIQSEHEQIDFIFFLLSSG |
| | | YLSTDYMSYRSIFIPGGLSETDNLFLKDVMSGKGPEKTFSFHLDNVNNIVERLKKLGVLQRD |
| | | NAQHPAVIRWLIDNDPDTLKNNIMALLSQTGSQRVVSLLMLMQNDFTTYVRLRYLEIFMSDE |
| | | HILNRLLAHLCASEERTPEQKFFVQEIAAHLLCLTEKSNIWQSVEINKRIGELIDSSPILIT |
| | | AVPKGYGDAFFEVLKDNTLSVSYIPGDVGDEKCSVIRKTAGAGLFKYSVSNLKNVYLCLTQD |
| | | KNEERMSFSLYPFHCLESLAISELTEILWTNIEDFILSVFIESEEIDRIPELLNSSEVSMTV |
| | | VEQIIAKMDFCINNLDDIINRSECADNNASGRNIYSMLLQHDRIFPSFDNIEHLLHDTSINT |
| | | SGELVQWVNEKHFEFEPSDIVINDTGIFNNFISELICSPVISEEALLKVLSNLNVVIIDVPE |
| | | NIPLRNAELLCSEKKLAPTVNVFTVLFNALSENVDDINRMNTLLGNLIAQRPEIITQEPEDI |
| | | FYIEGDFDEELASELFRHKLIGMNIKVAALRWLRDNKPGILDKSYLLSLDILAELSPWMGDD |
| | | DLRLTLLKRCLVAGDAGKDALCVVLNSFADESYHGLLPHDRFRKIPHSVDLWEVAELISNLG |
| | | FIQPPKMGSGRDEHKIVTTPVRYVRDVEFYD* (SEQ ID NO: 175) |
| 11 | A | MFLNDQETSTDLLYYTAIASTVVRLVDETSDAPITIGVHGDWGAGKSSVLKMLEAACEKKDK |
| | | THCIWFNGWTFEGFEDAKTVIIETIVEDLVASRPMSTKVAEAAKKVLRRIDWLKMAKKAGGL |
| | | AFTAFTGIPTFDQIKGMYELASDFLSAPQDKLSAADFKAFAEKAGGFIKEADTDSNTLPKHI |
| | | HAFREEFRALLDAAEEKLVVIVDDLDRCLPKTAIETLEAIRLFLFVEKTAFVIGADEAMIEY |
| | | AVKDHFPDLPQSTGPVSYARNYLEKLIQVPFRIPALGTAETRIYTTLLLAENALGSEDDNFK |
| | | ALLNKAREEMKRPWISRGLDREAVMAALNGKIPEWENALLFSLHVTPMLSSGTHGNPRQIKR |
| | | FLNSMMLRQAIADERGFGSDIKRPVLAKIMLAERFYPSVYGKLVQLVSNHPEGKPEALAEFE |
| | | ALVRGGKTAPKSRADSKENSSESEDVQNWLKIDWAIGWAKAEPALSGEDLRPYVFVTRDKHS |
| | | TLSNLVVSSHLIPIMEKLLGPKIGMVKIKGDLEKLSPPDADELFEMLSDKLFQEDSFNRKPR |
| | | GFDGLEYLVETQPHLQRRLIDFARRIPVKKAGGWLATRIAQSLVDPTLIEEYTKLIQEWAS |
| | | DENLSLSKSAKATLQLSGYQH* (SEQ ID NO: 176) |
| | B | MGTSKAYGGPVHGLIPDFVENPSPPTLPPVDPADDSTLDTPLIPPDSSGSGPLSTPKANFTR |
| | | YSRSGSRSSLGKAVAGYVRNGVGGAGRASRRMGASRAAAGGLLGLISDYQQGGATQALERFN |
| | | LGNLAGQSASTALLSLVEFLCPPGGSVDEGVARQAMLETIADMSDVGEENFDELTPDQLKEW |
| | | IGFVVHSIEGRLMADIGKNGIKLPDDIDAIVSIQEDLHDFVDGATRTQLREELRNLTGLSGD |
| | | AIDRKVEEIYTVAFELLAREGERLE* (SEQ ID NO: 177) |
| | C | MSHHTLVARLGTDDNSDLQLSRQSTHLTEINFLKENGKLDFGLGQALNGLSDLGLTPMDVSV |
| | | DLALLAATVTAADTRISRGHNAQDLWTREIALYIPVASPTLWNSQTGLLSRMLNFLTGDRWT |
| | | IHFRSRPVIEFIGLIQRSSKERSVNPTSVCLFSGGLDSFIGAIDLLSNGGTPLLISFIYWDT |
| | | TTSVYQQKCAQLLSERYGQSFSHVRARVGFEKTTIEGEDGENTLRGRSFMFFSLATMAADAL |
| | | GGPVTINVPENGLISLNVPLDPLRVGALSTRTTHPFYMARFNELLGNLGISAHLENPYAYKT |
| | | KGEMAIHCHDHAFLRQHAADTMSCSSPQSTRWNPALNEQQSTHCGRCVPCLIRRASLFTAFG |
| | | TDDTIYRIPDLRSRVLDSSKPEGEHVRAFQFALARLARSPSRAKFDIHKPGPLSDYPDCLAE |
| | | YEGVYLRGMKEVERLLSGVITRPLT* (SEQ ID NO: 178) |
| | D | MKLAGQKPAPQWVDFHCHLDLYPNHSALIRECDISRVATLAVTTTPKAWMRNRELTSDSPYV |
| | | RVALGLHPQLIAEREHEIALLEHYLPSARYVGEIGLDASPRFYRSFEAQERIFSRILNACFE |
| | | QGDKELSIHSVRAAAKVLGHLENTRLTENCKAVLHWFTGSISEARRAVELGCYFSINEEMLR |
| | | SPKHRKLVSFLPFERILTETDGPFVFHEEKAIHPRDVQRTVHEIAQIHHVSDTDAAMRILYN |
| | | LRSLVTNSSHSENSS* (SEQ ID NO: 179) |
| 12 | A | MSTVDTSTAEELNQGGSDFILTSLEAMRKKLLDLTSRNRLLNFPITQKGSSLRIVDELPEQL |
| | | YETLCSEIPMEFAPVPDPTRAQLLEHGYLKVGPDGKDIQLRAHPSAKDWAHVLGIRTDFDLP |
| | | DSHKTVVSDSDRELLEKAHQFELQYAQGQNGKLTGIRSEYVNQGIALSALKEACCLAGYEGL |
| | | EDFERQAKAGNEISISSSNPSHDDNRIQALLYPNELEACLRAIYGKAQTALEESGANILYLA |
| | | LGFLEWYESDSSEKARYAPLFTIPVRCERGKLDPKDGLYKFQLYYTGEDILPNLSLKEKLQA |
| | | DFGLALPLFNEEETPESYFASVKKVVEQHKPKWSVKRYGALSLLNFGKMMMYLDLDPARWPC |
| | | DKRNILSHEVIRRFFTSQSCGQENSGLPGGFGQHEYCIDSYPDIHDKVPLIDDADSSQHSAL |
| | | IDAIRGQNLVIEGPPGSGKSQTITNLIAAALLNGKKVLFVAEKMAALEVVKRRLDRAGLGQF |
| | | CLELHSHKTHKRKVLDDINARLVSQATMPTMEEIDAQILRYEDLKQQLNEYAALINNQWAQT |
| | | GKTIHQILSGATRYRHKLDIDATALHIENLSGKQLDKVTQLRLRDQIVEFSRIYKEVREQVG |
| | | ANAEIYEHPWSGVNNTQIQLFDSARTVDLLQTWQTSIIDFQHSYQEYVDKWALEGESLNTLQ |
| | | YIEQLVEDQSNLPVLCGSEHFPALSELDSPDAIARVRHYLDRFELLQGHYVALSQVIEPQKL |
| | | RLLEQGQSCDFPREELEKYGAAEDFTLRDLVRWLESIQSIHDELSSIYAQLNDFKNALPDGI |
| | | ASYIDDSQAGLLFCSELLSILGALPTELIRVRDPLFDDDDIDAVLRDLMCQIETLRPLRDGL |
| | | STLYQLDQLPSQEMLAHAVAVIQQGGLFAWFKSDWRSAKALLMAQSRKPDTKFAELKRCSAD |
| | | LLKYSELLQRFEQSDFGNQLGNAFRGLDTDCEQLMLLRDWYKKVRACYGIGFGKRVAIGSGL |
| | | FNLDGEIIKGVHLIEKSQISSRLMTLVKRVEHEAKLLPRISSLLEEHASWLGEQGVLMQSYR |
| | | QVRNTLIALQGWFINPDISLEQMTHSSEILQNINDLQISLENDSLQLGAFLQLTPACGAYKN |
| | | KQLTLDTINDTLNFAEQLVDKINCVSLATQIRHLASGSDYDLLCRDGGEIVSKWNEQIKNAE |
| | | LYALETKLERSQWLKSTDGSLNTLIERNERAIQQPRWLNGWVNFIRCYEQMHENGLQRIWSA |
| | | VLAGSLPIEKVELGLALAIHDQLAREVIHIHPELMRVSGSQRNALQKSFKEYDKKLIELQRQ |
| | | RIAAKIACRNIPEGNSGGKKSEYTELALIKNELGKKTRHIPIRQLVNRACNALVAIKPCFMM |
| | | GPMSAAHYLEPGRMEFDLVVMDEASQVKPEDALGVIARGKQLVVVGDPKQLPPTSFFDRSAD |
| | | GEDDDDAAALSDTDSILDAALPLFPMRRLRWHYRSRHEKLIAYSNRHFYNSDLVTFPSPNAE |
| | | SPEYGIKFTYVSKGRFSNQHNIEEAQAVAEAVLHHAHHRPGESLGVVAMSSKQRDQIERAID |
| | | ELRRNRPEFNDAIDGLHAMEEPLFVKNLENVQGDERDVIFISFTYGPSEHGGKVYQRFGPIN |
| | | SDVGWRRLNVLFTRSKKRMHVFSSMRSEDVLTSETSKLGVISLKGFLQFAESGKLDSLTTHT |
| | | GRAPDSDFEVAVMEALNHAGFECEPQVGVAGFFIDLAVKDPGCPGRYLMGIECDGAAYHSAK |
| | | SARDRDRLRQEVLERLGWRISRIWSTDWFSNPDEVLSPIIRKLHELKTLAPDVVVPSYEYVE |
| | | TIESSAEVASDSIDSLMPNLGLKEQLKYFATHVIEVELPNVDADRRLLRPAMLEALLEHQPL |
| | | SRSEFVERIPHYLRQATDVYEAQRFLDRVLALIDGAEAEANDAAFESELA* |
| | | (SEQ ID NO: 180) |
| 13 | A | MAGASIDAIGVINQIKDNLTDRYEDGFPVLKEIIQNADDAGANELTIGWSKGFCNAENELLN |
| | | APALFFINDAPLAEEHRDAILSIAQSSKATSKASVGKFGLGMKSLFHMGEAFFFMSDQWRIE |
| | | HWASDVFNPWDKYRDAWNEFGENDKCQIATKLKGFLSTDKPWFVVWVPLRTKALAKAHNNYI |
| | | IINNFSGDEKLPSFFNQAHLSEKTSEILPQLKNLKDIGFFCESDKGVFDEVTSIQLHEDSSR |
| | | SSFCGEPRLNNGDSFAVFSGKIYSNSNEERCALDYAGCERVIFDERLNQLKDENMGWPKSYQ |
| | | FDKKANLPVEALDKAEQHASVTFSRFKTKGQAYLKANWAVFLPLSQTKELVAVPIEGEYDYN |
| | | LYLHGYFFVDAGRKGLHGHDNLGFSTSLEHVKNDEKKLREVWNIILASEGTFNLVLPALNEF |
| | | CQKLRLPHQIKTVLTKALYDLLIERYRKEVSKSANWIINIDDKGAAWSLLDKNAQCLPIPRP |
| | | ENSDYSRIWSTLPGLSKLLDKKSLYEATGNEFLTEQNQRDSWNITLLEEALGSGVVNAFYRS |
| | | INIEYLLQFLQLAKEQCTTEDFDNLIIPQFREVLSTHKLAELSLNKALNTQVFELVSAPKTV |
| | | VLPIDKDDQSIWELVCKIIPAKLLLPKFLSTHNKPIHDNVTEEELFALLTLVDSYIKKQGER |
| | | LSSDESSACERLITFVIDCVNASEYIQKSDFYQKSGHLKLLKVEALGSQQSTKYRSLNELIV |
| | | LKEKYQLFLRGGERNFGKGLGKELVAVVPGLELCFISKDFEIGGLYEGLTACSEAACLRLLS |
| | | TYPNLGSNSARLALTKVFSAELSTDEEKRGFRYLIHGSKEDDLRQTLWKPNRATNPVWMKIW |
| | | RMCQPEDFPGWCELDEEFSNALTNQYEHFIGVKEQFYKDIISEYRTILPECNFDNFDDWEVE |
| | | QLLADIGSQGDERLWKALPVHRTAHNTRVAITTKCLMEGSATVPSEWDVHLIQHSAIAEVAA |
| | | CQHKWVNHGLPKELIEIALTQSSPAQYSAFILDQLCAIRIANEGIEHELEGKINNTKWLRLA |
| | | SGTEVSPEAILSFSANELPESAKFCELKESNIYMFSQLDGNMFEHDQARGFLREWVAKSNSS |
| | | VCSCILAEAAQHQSYVVGNFSNISAQVLEQISCIPPLMQLSAGWGLLVELYQSQYLSVNENK |
| | | QVMLCKETEPQSLWWALERIADDDIFIGQSKELRKAFLEALCNTEGGVDYLPKLRFRNENGS |
| | | YVSGNTLVSNVAQVVADNLISPQEYAVIESYCSKSALTNGNTSKIIELAGDNAPVLSDYFDD |
| | | WEGMVPPDAIATFIALFAKSGGVEKLVNNYLRQSTLESIKQGYEEKWNSGKGRRGEFSHYPY |
| | | SSLYKSVDFELAICAENAAYMTSIFGERIQVKLQKTPDSLLVHQANKSKTKRIELRRVDTKN |
| | | VSKDQLLRMLAKAVETIFTDVFGAECIRFESEFLKRFGASEQVDIQITRQIVLENVVPLLER |
| | | LQVREEGLCDLRSDYKREQRVLASSDPSVLQDRSRLNSVLTKIKETLENNEKVQSLVLESVR |
| | | KEMSKHFQYSPFSVPFELFQNADDALCELIEMQGDSTNVLTRFDVVSGSDGTLNFYHWGREV |
| | | NYCKSSYVAGKNQFDRDLEKMVSLNVSDKSDGKTGKFGLGFKSSLLLTDIPRLVSGDICAEI |
| | | HAGVLPSVPSKPVMTELNQNVDEYKIGNRKPTLIQLPKCDKKRADLKLVLGRFKSNAGILTV |
| | | FSRQIREINIDEQRFGWSGQALHNIPEVLVGEVKLPTNTSEESNVILRSNRVLIINTESGQF |
| | | LFALDSNGVVSLSNRKNLSSFWVLNPIDEDLKLGFCINAPFAVDIGRSQLAVDNGDNIDLSS |
| | | SLGKALSAVLVKMFAASSNNWNEFAEEVGLGQSSTFIKFWASLWDVITAHWPARLGETNSKA |
| | | ELKQMFTVEDGLLAFYQRCAALPRNLGVKEDSLVQLKNVDTGANKPLTKAFNTLGNHPILQR |
| | | LYKDQQLVGHDTFEFLKSIDFRPNNGALTKLELIDLIGQDFPHNEVNHDRASFYGRLFGKNF |
| | | EKLMSNFEMTVTEKKVLEERFSELKFLNKTGVYVTASKLIVEGSPERDLLSKFAPDSAKLSE |
| | | KYDQASMDLVSFIRRDVSYDIHSWAKQIRSEESNRGGKQEGLCSFLVEGGYLASSLLRKLQT |
| | | DHPAFLTKGRFDPSVLTEKWRWSSSKASAFISIWIDTEEDKARFIVRQAQKEFIPNVTNGEQ |
| | | ILENITNWWNQCRNQSLIDYDKQLYAQPMPWKAMTEDFELETLEVKKGWLKLFYLGSCQTLG |
| | | FNNDVANRNVVSWFEDKGWWDKLAVANGPSPEVWKELMEEYLQTARVDERYRVWIQVLPLYR |
| | | FATKLKDYVALFMNASFIDNLDDLLKPNSSNKLSGSGIQVSELKGTLGIGINFILRELQRHQ |
| | | VLEREYCEDIQKYAFVLPARLRKLLKKMGAGLSFDAEPENSERAYDYFVSALNSETHPLLKD |
| | | FDIPFRVLLADKQAFERCFNFALDEQFEEVYG* (SEQ ID NO: 181) |
| | B | MDNIIRVIHPKFGVGTVEFEKAETSLVRFEHGFEECLKSELEAVADLKSDLVSGQSVAASEL |
| | | ALKTLAHSLKSVNENWSVFSKSNINLLPHQLWVCHRVLRQWPTNQLIADDVGLGKTIEAGLI |
| | | LWPLIERKRVKRLLILTPAPLVEQWHQRMLDMFDIRLSMYAPENDTSRVNYWDSNNMVVASL |
| | | PTLRNDKNGRLERMLNAEPWDMLIVDEAHHLNSTEDKGGTLGFRFIQTLIENDKFESKLFFT |
| | | ATPHRGKEHGFFSLLQLLRPDLFNVKQMDEREMRPFVKDVLIRNNKQFVTDMNGERLFKPLS |
| | | VSSRTYSYSEQEQFIFYDLLTKFIVSGQAYASSLNSRDQRAVMLVLTAMQKLASSSIAAIER |
| | | ALKGRIEKHKLGKQRLQDIEVQQAALLEKREESESQSESEIYSDELAQLELEFIETTTRVQL |
| | | MDDELPRIMELLSACQKVGSETRILTILDILETEFKDRTVVFFTEYKATQALLMGALNKKYG |
| | | EGCVTFINGENRLLNVENGSGVCVDYVTDRYNAAKRFNEGKVRFIISTEAGGEGIDLQQNCF |
| | | SMIHVDLPWNPMRLHQRVGRLNRYGQVKNVEVITLRNPDTVESRIWDLLNTKIDLIMRSVGG |
| | | AMDEPENLMELILGMADSTLFNELFTEAANRKNSESLSAWFDHKTKTFGGESWQKVKDLIGR |
| | | AEKFDYQDLEAVPRLDLGDLKPFFTQMLSFNQRRCKYDENGGLSFLTPHAWLGQFGTRRSYE |
| | | KLHFDRKAKQLDSEADIIGFGHPMFSKAVNQGEQIPGSYAFLNGIEKDLVVFKVQDQVTGTD |
| | | ASVKVSIVGLVLDDNGDCELVKDEDLIGYLNEYLKISNDVDSKRTPEDLVSVIQTANDYLME |
| | | NVSSIGLPFRLPNSEPLTVFYKASN* (SEQ ID NO: 182) |
| 14 | A | MVAIKMYPAKDGDAFLIICDEEKSAFLIDGGYAETFRQHILPDLRELSFNGYRLRLVMATHI |
| | | DSDHIGGLVDFFLVNGHAAEPAVITVDRVWHNSLRAMTRPENNAQKVDSREITDFLRRRYHV |
| | | EADKAKPHEISARQGSSLAASLLAGDYHWNEGKGYQCICTGTSIPNLMCDNSLTILSPSKER |
| | | ISALCLWWRRQLASLGFSGRSSSSEAFDDAFEFFCKREASQVPLPHVINARTPLLERDYARD |
| | | TSPTNGSSIAFSLVLNKKRILMLGDAWAEEWTSLGASGASHHFDIIKISHHGSIRNTSPNLL |
| | | KIIDAPVYLISTDGKKHARHPNLAVLKAIVDRPAAFTRTLYFNYANSASAFMKNYLSASGAQ |
| | | FRIIEGSTDWITL* (SEQ ID NO: 183) |
| | B | MRYAATETEIRNATVLIECAGYTGSGTLIAADKVLTAAHCVVSDDPETPITVTFFGADEDVC |
| | | VNATISEIDTSCDACLLTLSDSVDIPPITLMTQPEREGSQWKAFGYPASRNGPSHYLHGTIS |
| | | QILPRLFHGVDMDLSVSADCVLEEYSGVSGAAILSENKCIAMVRIRMDGGLGAVSLDKLSGL |
| | | LIRNGLIPDDIASLPDSSLSGEVVLNRTEFRDNFESFVLEHKGRAVLLEGSPGSGKTTFCRH |
| | | YQPRSEQLAVAGVYEFTPEDGAGTTFKILPEVFADWLHNQVSILLSGRPARREETEKINLTQ |
| | | KVSDLLHTFSDYWKHKGKYGVIFIDAVNEASECGDEAVSRFTALLPVTLPENVKLVFTAPSL |
| | | SSAGKAFRHWLTPQDCISLTLLSHREVLQLTARELKTSAPSLSLLTRVSDIAQGHPLYLRYI |
| | | LGYLKANPDQVNLEIFPVFSGSIETYYERLWQGLVKDESAVNLLGILSRMRWGIDISSLIPV |
| | | LTPQEQTVFVPTLDRIQHLLLNDKSSALCHQSFAAFINSKTAVINSLLHGRLADFCLTSGES |
| | | YGLINRAYHLLLASHDRHPEAALVCTQEWADACIVKGAQPDELIHDIRQTLKNTLIRADAVA |
| | | SIRLLLLFQRMTFRHHFLFLQSAYHSGLALAALGRPDEALEQLIPSGSLVVDAVDAIVSAQT |
| | | LARMGNSEHALKLLEKVKSAVDQEFERNPVNLSDFIGLSLAWVRAELMAGVVDGHGRTREVV |
| | | EYLYGCGQVVRDNFEQSAHSKSAYTRAFYPLQAEMEAVNIAFNDRSVSLRTVKEKFGSLPEN |
| | | ILDLMLSSVMRAHDIILQHQLPMPQHALQPVWYNLDRLLHTDIPYSNEIRFNSLSSLIFFNA |
| | | PSALIIRMAGSFEVVPEITLLNEENEIAADSIDVSEQGQLWLVSAYLNETQPCPDIKHPSQG |
| | | CSEWLKTLTEAIFWYSGQARRAVIDGNDEKKELLLVKVQNDILPALSYSLEERMAWPNSWAM |
| | | PEQIIPMIYEELVNMFGACWPDKISVTTDFILAHTPQQCGLYSEGYTIRLLNRVIQTLLNEH |
| | | RFLGQSDTTFQLLETLFIAFVSAFTENRQELVPELLNIIPAYISLDAPQLAQDTYTELLGVS |
| | | MGPDWYKEDQFALMTTMLRVIPQHTDTNTTLSQVAGFLEHASGEMTFRRYVRQEKSQFIGEL |
| | | IRRGNYAHGFNYYRQQSCGSHEEMLTQLSHPAADSPHPLKGMRFPGGALDEEHAVECIVSEL |
| | | RNRVDWRLRWGLLEIFSFGSIGNLAVPFAELINEFSADTEDLNEIPKRLHNILHGDVPFSEH |
| | | RNFIKNFTEHLADNHKPLFAEFISLLSEDTSDNDVKPPPSGDANQKGTDTSDDVAMQPGLFG |
| | | KRSAINRAEACMENARKAAARRNTVRASELAVESLHIIQDGDWSVWRKNNHLAELTRTYILD |
| | | NSADAGSVIRAYASLVEKERYAPAWVIASHLTEIAASKFSDQEAQAINQIVLEHNRHMLGNT |
| | | EADAAHFSFLNEPDTSDAGEETLYFLFWLLEHPLKFRRERALEVLKWLASDDDKILGQCVTE |
| | | ALVSDIASRAEALMALTDWVSARSPQRIWDFIVKERSLFEWLEGTTALSQVHLLERVTSRAG |
| | | FVLRNEIAAFERPRKLLLTSEASGQRNIPENLPTWVQSLSQTLAVMEKQGIDIPALLTLLEK |
| | | RVLQQSGLADITVAFELEBCLLARGFTVNRTPSHHRWETMVRFALNQIIHEAAAQDELQNIE |
| | | PLLRAWNPASEECVEPWEVCNRAKQIICAVMEGRHQQASGIEDGFFLHYLDEVEVSREGQTH |
| | | LVEISAVLTTAHNGHESLRPGAESEFNATQTPDERTLSVHLTCQRVKMQPLLFGGATPAAVS |
| | | KKFMQMTGTLPSDFIRRQWRSGRSLSKNRWGEPISRGSLLLMKRTTTLPPGLGLAWYVTVDG |
| | | KLMNIFSYAPRRR* (SEQ ID NO: 184) |
| | C | MKYSMETPKTREEFEARCFHLLNAIKLGRYHGIPGEGNKEQVPFLPNGRVDLANIDTMTRLS |
| | | MNSLYDFHYNRDNYPQFDLSENDENEEATD* (SEQ ID NO: 185) |
| 15 | A | MSDSLLVRTSRDGDQFHYLWAARRALRLLEPQSTLVALTIEGASTTEMGSQPWEDGEELTDI |
| | | AEYYGSNELATATTVRYMQLKHSTMHSDTPFPPSGLQKTIEGFATRYKALIQKIPVETLRTK |
| | | LEFWFVTNRPVSSSFSEAINDAANQHVTRHPHDLAKLEKFTGLQGAELSIFCQLLHIEGQQD |
| | | DLWSQRNILLRESAGYLPDLDTEAPLKLKELVNRKALTESAANPSITRMDVLRALGVDETDL |
| | | FPAPCRIERIENSVSRTQEATLVQRVVEAFGAPVIIHADAGVGKSIFSTHIEEHLPTGSVSI |
| | | LYDCFGLGQYRNASSYRHHHRTALVQMANEMASRGLCHPLIPNAGTGISQYMRAFLHRLSQS |
| | | ISILRASEPLAVLCIIIDAADNAQMAAEEIGETRSFIKDLIREKLPDGVCLVALCRPYRREL |
| | | LDPPPEALTLSLQTFNRDETAAHLHQKFPDASESDVDEFHRLSSCNPRVQALSLSQNLPLND |
| | | TLRLLGPNPKTVEDTIGEVLEKSIARLRDTAGISERAQIDTICSALAILRPLIPLSVLSAIS |
| | | GVAGSAIKSFALDLGRPLIVSGETIQFFDEPAETWFQRRFRPSAADLHQFITKLRPLTKDSS |
| | | YAASVLPALMLEGNQLSELIELAISSQALPETSAVERRDIELQRLQFALKAALRTGRYQDAA |
| | | KLALKAGGECAGDNRQRVLLRDNIDLAAKFVGSNGVQELVSRNAFPDTGWPGSRNAYYAAIL |
| | | SEYPELSGEARSRLRLTMEWLTNWSQLPDDERSRQNVTDQDRAVMLIACLNIHGAEAAAREL |
| | | RRWRPRKLSFDAGKIVAMQLLAHARYDELDQLAIAAGNDISLVMGIVLEARKLHRPVAEQAI |
| | | RRTWRLLKSQRVSIKDRNHANNQTIAAITGMVEMALIQSVCTESESIQLLDRYLPKVPPYAL |
| | | TSEYSKERVAYVRAYALQANLMGSQLALSDLASTEVKKELMAEKRHGESDDLRQLKQYSGVL |
| | | IPWYNLWAKVILGKTRKADLESELSDTQKESTAIKGHSYSEHSLSSNEIANVWFDILEAGNV |
| | | SKDDVENIIKWSQHKGNRVFTPTLHRFSSVCAEISGLGELSYHFAELALSLWRDEHSDAQIK |
| | | ADGYIDLSRSLISLDEPEAKEYFNQAIEVTNKLGDENLSRWEAILDLAEYVAGKTQVPPETS |
| | | YKLARCAELTREYVDRDKHFAWSDTVEILAELCPSSALAIISRWRDRTFGNHRSILAWTIEH |
| | | LVKKNKINALDALPLITFENDWHKCDLLDSVLSSCTDDKDKIMAFEVVYHYTKFNVQNIQNL |
| | | KKLDAISTSLGIEHTELKERISGLQHTETVSKKSSLSSNDNEQGHDQEWESIFKDCDLSSID |
| | | GISAAYEKFRNVPEFYSKETFIKKAISRVKTGKECSFITAIGAIFHWGLYDFKYILESIPDE |
| | | WTSRLSIKTTLAGLIKEYCQRFCMRIRKSRVYEIFPFSLASRLSGISEKEIFGITLEAIAES |
| | | PEPANSDRLFSLPGLLVSKLESNEALDVLSYALDLFDEVLKDEDGDGPWNEKLSPPTHVEDS |
| | | LAGYIWARLGSPEAEMRWQAAHAVLALCRMSRTCVIQGIFQHAINATTLPFCDRNLPFYTLH |
| | | AQLWLMIAAARVALDDGKSLIPNIGYFYHYATTDQPHVLIRHFAARTLLALHDSDLISIPAQ |
| | | EENKLRNINQSTTLPVLDKVEDHRGEDSYTFGIDFGPYWLKPLGRCFGVSQKQLEPEMLRII |
| | | RDVLGFKGSRNWDEDERNKRRYYQDRDNHHSHGSYPRVDDYHFYLSYHAMFMTAGQLLATKP |
| | | LVGSDYDDVEDVFQDWLRRHDISRNDHRWLADRRDIPPKERSSWLNSSSDNRDEWLASISEN |
| | | VFNETLCPSPGLLTLWGRWSDVCSDRKESIIVHSALVSPERSLSLLRALQTTKNVYDYKIPD |
| | | AGDNLEIDHAHYQLKGWIKDIAEYCGEDEFDPWAGNVRFPIPEPASFIIDAMKLTTDKDHRW |
| | | VTSPSDVEPAMISSIWGHLSGKNDEEKSHGYRLCASIHFIKSALETFNMDLILEVDVDRYSR |
| | | NSRYERNNENELDNIPSSTRLFLFRHDGTIHTLYGNYRNGEKTS* (SEQ ID NO: 186) |
| | B | MAHHIAELIYDAEHCTDDIVRTAKQAEIRDSIWSFWSNRYELPIGSRPFQELEPILRTLKGL |
| | | DPENEQPRFFSPYRDLINVEKETSEVQKWLTAAKDIDSAAKILIDYCLSLAAENAIDKSQEW |
| | | VELAQKAGLNKDVDLLEIRIFQLRGTPANTDNPNNAQRRILEKRQKRLEAFLLLGSQLNEQL |
| | | KSQLEALPAIEDEPTDDDEDF* (SEQ ID NO: 187) |
| 16 | A | MEPISITVATYVATKLIDQFISQEGYGCIKKALFPQKRYVDRLYQLIEETAIEFEETYPVES |
| | | GAIPFYHSEPLFEMLNEHIFFKEFPDKEILLDKFKEYPSITPPTQQQLSLFYEMLSLKINNC |
| | | SKLKKLHIEETYKEKIFDINEELIQVKLILRSIDEKLTFHLSDDWLNEKNSQAIADLGGRYT |
| | | PELNVKLEIAEIFDGLGRTNDFSKIFYSHIDSFLVAGKKLHSCDVISSELFEINQSLKEISD |
| | | IYQEINFSKLDEIPINKFNNYVSSCQTAIGGAVSILWELREKSEQVGETKHYSDKYSSTLRM |
| | | LREFDYACNELRIFINSTTVKLANNPFLLLEGKAGIGKSHLLADVIKNRIASGYPSLLILGQ |
| | | QLTSDESPWSQIFKRLQLKITSREFLEKLNLYGKKTGKRVLVFIDAINEGNGNKFWNDNINS |
| | | FVDEIRCFEWLGLIMSVRTTYRNVTISHENVVRNNFEIHEHIGFQNVELEAVSLFYDYYNIE |
| | | RPSSPNLNPEFKNPLFLKLLCEGIKKNGLTKVPVGFNGISNIFNFLVEGVNKSLASPKKYAF |
| | | DPSFPLVKDALNEIIKFKLEIGRNSISLKDAHSVVQSVVNDYVADKTFLSALIDEGLLTKGI |
| | | VRNDDNSTEEVVYVAFERFDDHLTVNFLLNDVENIESEFKPDGRLKKYFHDECDFYIKSGIV |
| | | EALSIQLPERYEKELYEFLPEFSNNLKLLEAFIDSLIWRDIKAIDFEKIRPFINEHVFKFKD |
| | | SFDHFLEAVISISGLVGHPFNANFLHDWLKDYSLANRDSFWTTELKYKYSEDSAFRHLIDWA |
| | | WARTDKSFVSDESIELVATSLCWFLTSSNRELRDCSTKALVSLLEPRIPVLRKIIDKFYGVN |
| | | DPYVWERIFAVALGCTLRTDNIKELKYLAETVYQKVFCSKYVYPNILLRDYAREIIEFANHL |
| | | GLELESIELSKTRPPYNSIWPDKIPSKEELESLYDKEPYRELWSSIMEDGDFSRYTIGTNYN |
| | | HSDWSGCKFNETPVDRKQWKTFKCKLTDQQKDLYDATDPFIYDDKCEGIKFGRVVGRKAQEE |
| | | IKASKKLFKNSLSYDLLSEFENEIEPYLDHNNNLLETDKHFDLRLAQQFIFNRVIELGWDPE |
| | | KHGNFDQQIGTGRGRREAFQERIGKKYQWIAYYEYMARLADNFTRFEGYGDERKENPYQGPW |
| | | EPYVRDIDPTILLKETGTKPGSNKEMWWLNDEVFDWTCSNEDWVKSSTTITNSYAFIEVKDD |
| | | NGDEWIVLESHPSWKEPKIIGNDDWGHPRKEVWYQIRSYIVKVEEFENFRCWAIAQDFMGRW |
| | | MPECTDRYQLFNREYYWSEAFKSFKSDYYGGSDWTSVTDRESGAKIADVSVTSINYLWEEEF |
| | | DKSKIETLNFLKPSNLIFEKMGLKSGEVEGSFNDENGTMVCFAAEAVYASKPHLLVKKEPFL |
| | | TMLRDNGFEIVWTLLGEKGVIGGSLISSHHYGRQEFSGAFYYEDSQLTGSHKTSFTR* |
| | | (SEQ ID NO: 188) |
| 17 | A | MVKPNWDNFKAKFSENPQGNFEWFCYLLFCQEFKMPAGIFRYKNQSGIETNPITKDNEIIGW |
| | | QSKFYDTKLSDNKADLIEMIEKSKKAYPGLSKIIFYTNQEWGQGRKSHEPEGDKNADNYLET |
| | | VGNSNDPKIKIEVDQKAYESGIEIVWRVASFFESPFVIVENEKIAKHFFSLNESIFDLLEEK |
| | | RKHTENVLYEIQTNIEFKDRSIEIDRRHCIELLHENLVQKKIVIVSGEGGVGKTAVIKKIYE |
| | | AEKQYTPFYVFKASEFKKDSINELFGAHGLDDFSNAHQDELRKVIVVDSAEKLLELTNIDPF |
| | | KEFLTVLIKDKWQVVFTTRNNYLADLNYAFIDIYKITPGNLVIKNLERGELIELSDNNGFSL |
| | | PQDVRLLELIKNPFYLSEYLRFYTGESIDYVSFKEKLWNKIIVKNKPSREQCFLATAFQRAS |
| | | EGQFFVSPACDTGILDELVKDGIVGYEAAGYFITHDIYEEWALEKKISVDYIRKANNNEFFE |
| | | KIGESLPVRRSFRNWISERLLLDDQSIKPFIAEIVCGEGISNFWKDELWVAVLLSDNSSIFF |
| | | NYFKRYLLSSDQNLLKRLTFLLRLACKDVDYDLLKQLGVSNSDLLSIKYVLTKPKGTGWQSV |
| | | IQFIYENLDEIGIRNINFILPVIQEWNQRNKVGETTRLSSLIALKYYQWTIDEDVYLSGRDN |
| | | EKNILHTILHGAAMIKPEMEEVLVKVLKNRWKEHGTPYFDLMTLILTDLDSYPVWASLPEYV |
| | | LQLADLFWYRPLKETGERYFISMDIEDEFGLFRSHHDYYPESPYQTPIYWLLQSQFKKTIDF |
| | | ILDFTNKTTICFAHSHFAKNEEEVDVFIEEGKFIKQYICNRLWCSYRGTQVSTYLLSSIHMA |
| | | LEKFFLENFKNADSKVLESWLLFLLRNTKSASISAVVTSIVLAFPEKTFNVAKVLFQTKDFF |
| | | RFDMNRMVLDRTHKSSLISLRDGFGGTDYRNSLHEEDRIKACDDVHRNTYLENLALHYQIFR |
| | | SENVTEKDAIERQQVLWDIFDKYYNQLPDEAQETEADKTWRLCLARMDRRKMKITTKEKDEG |
| | | IEISFNPEIDPKLKQYSEEAIKKNSEHMKYVTLKLWASYKREKDERYKNYGMYEDNPQIALQ |
| | | ETKEIIKKLNEEGGEDFRLLNGNIPADVCSVLLLDYFNQLNNEEREYCKDIVLAYSKLPLKE |
| | | GYNYQVQDGTTSAISALPVIYHNYPMERETIKTILLLTLFNDHSIGMAGGRYSVFPSMVIHK |
| | | LWLDYFDDMQSLLFGFLILKPKYVILSRKIIHESYRQVDYDIKKININKVFLNNYKHCISNV |
| | | IDNKISIDDLGSMDKVLHILNTAFQLIPVDTVNIEHKKLVSLIVKRFSTSLLSSVREDRVDY |
| | | ALRQSFLERFAYFILHAPVSDIPDYIKPFLDGFNGSEPISELFKKFILVEDRLNTYAKFWKV |
| | | WDLFFDKVVTLCKDGDRYWYVDKIIKSYLFAESPWKENSNGWHTFKDSNSQFFCDVSRTMGH |
| | | CPSTLYSLAKSLNNIASCYLQGITWLSEILSVNKKLWEKKLENDTVYYLECLVRRYINNERE |
| | | RIRRTKQLKQEVLVILDFLVEKGSVVGYMSRENIL* (SEQ ID NO: 189) |
| 18 | A | MQVQHHTEPNLKNEIVALFKASQLIPFFGSGFTRDIRAKNGKVPDAIKFTELIRNLAAEKEG |
| | | LTQTEIDEILRISQLKKAFGLLNMEEYTPKRKSKALLGNIFSECKLSDHEKTKIINLDWPHI |
| | | FTFNIDDAIENVNRKYKELHPNRAVQREFISANKCLFKIHGDITEFIKYEDQNLIFTWREYA |
| | | HSIEENKSMLSFLSEEAKNSAFLFIGCSLDGELDLMHLSRSTPFKKSIYLKKGYLNLEEKIA |
| | | LSEYGIEKVITFDTYDQIYQWLNNTLQNVERKSPTRSFELDDSKLMKEEAINLFANGGPVTK |
| | | IVDNKRILRNSITFSQRDVCDDAIKALRNHDYILITGRRFSGKSVLLFQIIEAKKEYNASYY |
| | | SSTDTFDPSIKNSLIKFENHIFVFDSNFFNAQSIDEILTTRVHPSNKVVLCSSFGDAELYRF |
| | | KLKDKKILHTEIQIKNNLINEEGNYLNDKLSFEGLPLYKSSETLLNFAYRYYSEYKNRLSGS |
| | | NLFNKQFDEDSMFVLILIAAFNKATYGHINSHNKYFDIQNFISQNDRLFELESTNTDPSGVI |
| | | ICNSPSWLLRVISEYIDKNPASYKTVSDLIISLASKGFLAASRNLISFDKLNELGNGKNVHK |
| | | FIRGIYKEIAHTYREDMHYWLQRAKSELISAHTIDDLVEGMSYASKVRLDSAEFKNQTYYSA |
| | | TLVLAQLSARALSINNDKIYALSFFESSLESIRNYNNNSRHINKMMDKNDGGFRYAIQYLKD |
| | | NPLIELLPRKDEVNELINFYESRKK* (SEQ ID NO: 190) |
| 19 | A | MQFITNGPDIPDELLQAHEEGRVVFFCGAGISYPAGLPGFKGLVELIYQRNGTTLSEIEREV |
| | | FERGQFDGTLDLLERRLPGQRIAVRRALEKALKPKLRRRGAIDTQAALLRLARSREGALRLV |
| | | TTNFDRLFHVAAKRTGQAFQAYVAPMLPIPKNSRWDGLVYLHGLLPEKADDTALNRLVVTSG |
| | | DFGLAYLTERWAARFVSELFRNYVVCFVGYSINDPVLRYMMDALAADRRLGEVTPQVWALGE |
| | | CEPGQEHRKAIEWEAKGVTPILYTVPAGSTDHSVLHQTLHAWADTYRDGIQGKKAIWKHALA |
| | | RPQDSTRQDDFVGRMLWALSDKSGLPAKRFAELNPAPPLDWLLKAFSDERFKYSDLPRFCVS |
| | | PHVEIDPKLRFSLVQRPAPYELAPQMSLVSGCVSASKWDDVMSHIARWLVRYLGDPRLIIWI |
| | | AERGGQIHDRWMFLIESELDRLAALMRERKTSELDEILLHSPLAIPGPPMSTLWRLLLSGRV |
| | | KSPLQNLDLYRWQNRLKNEGLTTTLRLELRGLLSPKVMLRRPFRYSEDDSSSTDEPLRIKQL |
| | | VDWELVLTADYVRSTLFDLADESWKSSLPYLLEDFQQLLRDALDLLRELGESDDRHDRSHWD |
| | | LPSITPHWQNRGFRDWVSLIELLRDSWLAVRAKDSDQASRIAQNWFELPYPTFKRLALFAAS |
| | | QDNCIPPERWVNWLLEDGSWWLWATDTRREVFRLFVLQGRHLTGIAQERLETAILAGPPREM |
| | | YEDNLEADRWHYLVAHSVWLCLAKLRGAGLVLGESAATRLTEISTAYPKWQLATNERDEFSF |
| | | IWMSGTGDPGFEESIDVDIAPRKWQELVQWLAKPMPERLPFYEDTWSDVCRTRFFHSLYALR |
| | | KLSQDDVWPVGRWREALQTWAEPGMILRSWRYAAPLVLDMPDAVLQEISHAVTWWMEEASKT |
| | | ILCHEETLLALCRRVLMIETSPESSTIRNGIETYDPVSTAINHPIGHVTQSLITLWFKQNPN |
| | | DNDLLPVELKTLFTKLCNVQIELFRHGRVLLGSRLIAFFRVDRPWTEQYLLPLFAWSNPVEA |
| | | KAVWEGFLWSPRLYEPLLIAFKSDFLESANHYSDLGEHRQQFAIFLTYAALGPTEGYTVEEF |
| | | RTAISALPQEGLEVAAQALYQALEGAGDQREEYWKNRVQPFWQQVWPKSRNLATPRISESLT |
| | | RMVIAARGEFPAALAVVQDWLQPLEHLSYDVRLLLESDICSRYPADALSLLNAVTAEQHWGP |
| | | RELGQCLLQIVQAAPQLEQDVRYQRLNEYSRRRSV* (SEQ ID NO: 191) |
| 20 | A | MTNKNKIKPLLNNISARLWDGRAAILIGAGFSRNAKPLTSKARKFPMWNDLGDIFYESVYCK |
| | | KNDNRYSNVLKLGDEVQAAFGRATLDKLIMDHVPDKEYEPSKLHVSLLSLPWIDVFTTNYDT |
| | | LLERASVNVDSRKYDIVLNKNDLMNAERPRIIKLHGSFPSERPFIVTEEDYRKYPLENSPFV |
| | | NTVQQSLIENTLCLIGFSGDDPNFLNWIGWIRDNLGTENSPKIYLIGLFSFNEAQRKLLEKR |
| | | NISIVDLSFLGDFGKDHYLAHQRFIQFLYESKNRDNLIEWPIETNYDRIVFNDGIELKTEKI |
| | | KKCILEWAQSRQSYPNWLILPESNRSNLWQNTIDWLSVANYDVAWDGSDDLDFGYEITWRLN |
| | | KALLPIFNDTSEFLFKLIEKYEINYVSGINNKIIDFDEKYSHITLSLMRFCRQENLIDKWKN |
| | | LNDLLIQNLDRLTPEVKSDYYYENILFSYFNLNFDEARNKLSNWETNKLLPHHEIKRAGLLA |
| | | EFGMLDEAINLLEETLSTIRRNSLLSSRNIDYSSESQEAYGIYILRMFKRSLRLDSKDDDYS |
| | | SEYNSRLATLSQYRSDPENEIKYLEIKLESLPGTFKNTNDTDFDLNKRTVTTYLGGSPTEVR |
| | | SLDAFSFFLLAEELGLPFHIPGMNIFSGIVENAARHIYQYSPEWAIFSIFRTFNKDKAKSLF |
| | | NRNRISSLERKKVEDLFDGYYKKYEQIITKKIEDRLNDKLEIEISTLSIIPEILSRLVTKVS |
| | | FNKKKDIIHLLLKLFNSDNFHQYMETKDLLKRTTSNLSDLQKISLIDEFIDFPSAPPNTQLH |
| | | MGQRYNFLTPFECLLGVTITPPKENSKKIASAKLKKDINDLKSDNLDLRKAVSQKLITLYNL |
| | | EMLNKSDTTKLIKNLWSKRDNFGFPIGSGYYKFFFINNLNPDNENIADKFISIIKTYKFPVQ |
| | | EGKRVSITGGLDEYCTELNGALHHISLPEKTLSEIISKIHDWYVKDRAWLEKRDDLAKEFTL |
| | | RFRNITNIITTILEHHKDKLHAESINEISSLLDKMKEDKIPVNSAVTMLCLKNKSTYLERKD |
| | | IENGLYSFNKDDVIEAINSTYVFIRNNEFPLTIIQAISDKIAWDRNPRLPDCYNLIAYIINS |
| | | CEFTLPDYLIEKILRGLAYQINIDDRDFVDNNEYLNHLEKKLSATKLAASMFRKNETLGIDQ |
| | | PSIIQEWKNMCNSRNEFDEIRNEWNNNI* (SEQ ID NO: 192) |
| 21 | A | MSIYQGGNKLNEDDFRSHVYSLCQLDNVGVLLGAGASVGCGGKTMKDVWKSFKQNYPELLGA |
| | | LIDKYLLVSQIDSDNNLVNVELLIDEATKFLSVAKTRRCEDEEEEFRKILSSLYKEVTKAAL |
| | | LTGEQFREKNQGKKDAFKYHKELISKLISNRQPGQSAPAIFTTNYDLALEWAAEDLGIQLFN |
| | | GFSGLHTRQFYPQNFDLAFRNVNAKGEARFGHYHAYLYKLHGSLTWYQNDSLTVNEVSASQY |
| | | DEYINDIINKDDFYRGQHLIYPGANKYSHTIGFVYGEMFRRFGEFISKPQTALFINGFGFGD |
| | | YHINRIILGALLNPSFHVVIYYPELKEAITKVSKGGGSEAEKAIVTLKNMAFNQVTVVGGGS |
| | | KAYFNSFVEHLPYPVLFPRDNIVDELVEAIANLSKGEGNVPF* (SEQ ID NO: 193) |
| | B | MSLFKLTEISAIGYWGLEGERIRINLHEGLQGRLASHRKGVSSVTQPGDLIGFDAGNILVVA |
| | | RVTDMAFVEADKAHKANVGTSDLADIPLRQIIAYAIGFVKRELNGYVFISEDWRLPALGSSA |
| | | VPLTSDFLNIIYSIDKEELPKAVELGVDSRTKTVKIFASVDKLLSRHLAVLGSTGYGKSNFN |
| | | ALLTRKVSEKYPNSRIVIFDINGEYAQAFTGIPNVKHTILGESPNVDSLEKKQQKGELYSEE |
| | | YYCYKKIPYQALGFAGLKLLRPSDKTQLPALRNALSAINRTHFKSRNIYLEKDDGETFLLYD |
| | | DCRDTNQSKLAEWLDLLRRRRLKRTNVWPPFKSLATLVAEFGCVAADRSNGSKRDAFGFSNV |
| | | LPLVKIIQQLAEDIRFKSIVNLNGGGELADGGTHWDKAMSDEVDYFFGKEKGQENDWNVHIV |
| | | NMKNLAQDHAPMLLSALLEMFAEILFRRGQERSYPTVLLLEEAHHYLRDPYAEIDSQIKAYE |
| | | RLAKEGRKFKCSLIVSTQRPSELSPTVLAMCSNWFSLRLTNERDLQALRYAMESGNEQILKQ |
| | | ISGLPRGDAVAFGSAFNLPVRISINQARPGPKSSDAVFSEEWANCTELRC* |
| | | (SEQ ID NO: 194) |
| 22 | A | MDRSAVDTIRGYCYQVDKTIIEIFSLPQMDDSIDIECIEDVDVYNDGHLTAIQCKYYESTDY |
| | | NHSVISKPIRLMLSHFKDNKEKGANYYLYGHYKSGQEKLTLPLKVDFFKSNFLTYTEKKIKH |
| | | EYHIENGLTEEDLQAFLDRLVININAKSFDDQKKETIQIIKNHFQCEDYEAEHYLYSNAFRK |
| | | TYDISCNKKDRRIKKSDFVESINKSKVLFNIWFYQYEGRKEYLRKLKESFIRRSVNTSPYAR |
| | | FFILEFQDKTDIKTVKDCIYKIQSNWSNLSKRTDRPYSPFLLFFIGTSDANLYELKNQLFNE |
| | | DLIFTDGYPFKGSVFTPKMLIEGFSNKEIHFQFINDIDDFNETLNSINIRKEVYQFYTENCL |
| | | DIPSQLPQVNIQVKDFADIKEIV* (SEQ ID NO: 195) |
| | B | MSRNNDINAEVVSVSPNKLKISVDDLEEFKIAEEKLGVGSYLRVSDNQDVALLAIIDNFSIE |
| | | VKESQKQKYMIEASPIGLVKNGKFYRGGDSLALPPKKVEPAKLDEIISIYSDSIDINDRFTF |
| | | SSLSLNTKVSVPVNGNRFFNKHIAIVGSTGSGKSHTVAKILQKAVDEKQEGYKGLNNSHIII |
| | | FDIHSEYENAFPNSNVLNVDTLTLPYWLLNGDELEELFLDTEANDHNQRNVFRQAITLNKKI |
| | | HFQGDPATKEIISFHSPYYFDINEVINYINNRNNERKNKDNEHIWSDEEGNFKFDNENAHRL |
| | | FKENVTPDGSSAGALNGKLLNFVDRLQSKIFDKRLDFILGEGSKSVTFKETLETLISYGKDK |
| | | SNITILDVSGVPFEVLSICVSLISRLIFEFGYHSKKIKRKSNENQDIPILIVYEEAHKYAPK |
| | | SDLSKYRTSKEAIERIAKEGRKYGVTLLLASQRPSEISETIFSQCNTFISMRLTNPDDQNYV |
| | | KRLLPDTVGDITNLLPSLKEGEALIMGDSISIPSIVKIEKCTIPPSSIDIKYLDEWRKEWVD |
| | | SEFDKIIEQWSKS* (SEQ ID NO: 196) |
| 23 | A | MAYEAQISRTNPAAFLFVVDQSGSMSDKMSSGRSKAEFVADALNRTLMNLITRCTKSEGVRD |
| | | YFEIGVLGYGGQGVSNGFSGSLGGQVLNPISALEQNPARVEDRKRKMDDGAGGIIETAIKFP |
| | | VWFDPIASGGTPMREALTRAAEELVTWCDAHPDCYPPTILHVTDGESNDGDPEEIANHLRQI |
| | | RTNDGEVLILNIHVSSLGNDPIRFPSSDTGLPDAYAKLLFRMSSPLPEHLVRFAQEKGHTVG |
| | | IESRGFMFNAEAAELVDFFDIGTRASQLR* (SEQ ID NO: 197) |
| | B | MKLEFLGTVPKDPEYPKANEDKFAFSEDGRRLALCDGASESFNSKLWADLLARKFTADPKVN |
| | | PEWVASALAEYSATHDFRSMSWSQQAAFERGSFATLIGVEEFEEHQAVEILAIGDSITMLVD |
| | | CGKLICAWPFDNPEKFNERPTLLATLYAHNNFVGGSTFWTRHGKTFYLEKLTQPKLLCMTDA |
| | | LGEWALKQALAEDSGFIELLSLQTEEELAELVLRERAAKRMHIDDSTLLVLSF* |
| | | (SEQ ID NO: 198) |
| | C | MPYPSLEQYNQAFQLHSKLLIDPELKSGTVATTGLGLPLAISGGFALTYTIKSGAKKYAVRC |
| | | FHRESKALERRYEAISRKISSLRSPYFLDFQFQPQGVKVEGISYPIVKMAWAKGETLGEFLE |
| | | VNRRSAQAIAKLSASIESLAAYLEKEKIAHGDFQTGNLMVSDGGATVQLIDYDGMFVDEIKT |
| | | LGSSELGHVNFQHPRRKATNPFNHTLDRFSLISLWLALKALQIDPSIWDKSNSELDAIEFRA |
| | | NDFVDPGSSSILGMLSGIQQLSTHVKNFAAVCASAMEKTPSLGDFIASKNIPISLASISMNG |
| | | DIPVSRLKPGYIGAYTVLSALDYSACLQRVGDKVEVIGKIIDVKLNKTRNGKPYIFVNFGDW |
| | | RGNIFKISIWSEGISALPSKPDASWIGKWISVIGLMEPPYVSGKYKYSHISITVTTIGQMTV |
| | | LSEPDARWRLAGPNESRQTLTSTSSNQEALERIKSKSTTSTPMPMNTNATTANQAILNKLRA |
| | | STQTVAAARAQTQHWPNKSSTHYVAPTGTSASQPVQNIPSPASTSKQQTSQKNIVTKILKWL |
| | | FG* (SEQ ID NO: 199) |
| 24 | A | MVGSRWYKFDFHNHTPASHDYKIPDISPREWLLAYMKQHVDCVVTSDHNSGAWVDVLKGELE |
| | | NMSRDASTGDLPEFRPLTLFPGVELTATGNVHILAVLHTHSTSADVERLLAQCNNNSPIPSE |
| | | VPNHQLVLQLGPAGIISNIRRNPKAVCILAHIDAAKGVLSLTNQAELTAAFQESPHAVEIRH |
| | | RVEDITDGTRRRLIDNLPWLRGSDAHHPEQAGVRTCWLKMSSPDFDGLRHALLDPENCVLFD |
| | | QLPPEEPASYLRSLKFRTRHCHPVGQDSASVEFSPFYNAVIGSRGSGKSTLIESIRLAMRKT |
| | | EGLTATQGSKLDQFIRTGMEADSFIECIFHKEGTDFRLSWRPDSKHELHIFSDGEWMPDSHW |
| | | SADRFPLSIYSQKMLYELASDTGAFLRVCDESPVVNKRAWKERWDQLEREYLNEQITLRGLR |
| | | ARQGSADSLRGELSDAERAVSQLQSSAYYPVCRQLALARNELSAATLPLEHFERRIAAIQAL |
| | | AEEPLQRSDIPPEPSGLLMAFMARLSSVQQQYDQRLNTLLAEYAAELAGIRREQSFIALRTA |
| | | VSDQETNVESEAVSLRARGLNPDVLNELMARCESLKNELRNYDGLDGAISASVARSEQLLAE |
| | | MRAHRMALTDNRKAFLSSLSLSALEIKILPLCAPYEDVISGYQTVTGISNFAERIYDNSDGS |
| | | GLLSDFISERPFSPLPAATEKKYRALDELKALHHSIRLDNSEAGAGLHGSFRNRLRSLNDQQ |
| | | LDALQCWYPDDGIHIRYQTPGGQMEDIAFASPGQKGASMLQFLLSYGTDPLLLDQPEDDLDC |
| | | LMLSMSVIPAIMSNKKRRQLIIVSHSAPIVVNGDAEYVISMQHDRTGLYPGLCGALQEAPMK |
| | | ALICRQMEGGEKAFRSRYERILS* (SEQ ID NO: 200) |
| 25 | A | MNEHLSHMDVHTLFEEMDEQADGITFKYSFDDIAKSNALVVTEFVNFERDSTVALLASLLTL |
| | | PAHQSQCLRFELLTSLALIHCKGQQIANIDDVKRWYVTTGESSSIVGEDPAEDWVALVDNKK |
| | | GDYRVLEGVWEAAGFYTQLMVEIVSDMPDTHRYRSLKLAIQAILRLSDVICARSGLYRFQEG |
| | | ADEFPDSLDTAGLDEKTLCSRVTLSERSLRAEGIKLADLAPFILEPSHISMLGNQVPGEGML |
| | | EQRPLLRTRDGIVVVLPTAMTIALRQAVITFAKRTEELSELDKALANVYSLTFSEMPVFGNG |
| | | GRLRRLTWEKYKMSRTTMVTSIVDAGHLMVLQFVLPSIQQYADTGFNNLLQLDEETTQFLDN |
| | | SVEQITVDLAKQPGFQRGIVVRIACGWGAGFMGVPPQLPDGWGFEWMSGADFVRFGALPDMS |
| | | PIAFWRVQDAVETIRQAGVRLINMSGTLNLLGWIRANDGHMVPHDQLPDDRITPEHPLMLMI |
| | | PTNLLRGIRIAADTGYDRHRISDNNGKWHRVMRPSAEDFFPTERQSKCYASIDDLEAQRLTC |
| | | VYEGQGNLWVTLEAPEMEDWMLLVELAKMVRTWIGRIGEALEVLSEQPIKKSLKVYLHFDGN |
| | | DNIGRFDGENSDDMNTFWRLERIHEHGAIRVVLQDGYLAGFRLPDNRAERALVRALGTAFAT |
| | | LLRMKEPVDKGVTVEQIAVPNDRARSFHIMQAYDFNQYLGRSLTKRLLAEDIDSAAARELAW |
| | | RAVSTDAPSRYQGKKEVGKLLNDWDVLIQDLLSELSRFDRKQTVMRLLENVVKARCEEAHWR |
| | | STAAAVLGLHAGEEGVEETIAQEMSRYAGAALTSRLIIELAICVCPTSGGIEPSDMALSKLL |
| | | ARASLLFRIGGMSDAVRFGALPADIRISPLGDLLFRDELGKMVLEPMLSKVTNERFEEQAAQ |
| | | FEQHYVKTAGGDDENSKQDSVAAETTEDQTDIFLAFWKAEMGFTLEDGMRFIQFLESIGEQE |
| | | SAEEMRRSQLADAAKSAGLADETIDAFLNQFILSARPKWDVVPDGFDLSDIYPWRFGRRLSV |
| | | AVRPLLQEESHDPLIVIAPGLLNLSLKYVFDGAYTGQFKRDFFRTEGMRDTWLGGAREGHTF |
| | | EKTLERELRETGWTVRRGIGFPEERRNLPGDPGDIDLLAWRSDRNQVLVECKDLSLARNYSE |
| | | VASQLSEYQGDDIKGKPDKLKKHLKRVLLAKENIDNFAKFTSIANPEIVSWLVFSGASPIAY |
| | | AQSKEALAGTNVGRPSDLLNF* (SEQ ID NO: 201) |
| 26 | A | MDYLSEVLKIIEGATKANASMASNYAGLLADKLEQKGEVKQARMIRERLLRAPQALAGAQRA |
| | | GGGISLGSLPVDIDSRLNTVDVSYPKLDSSEIFLPAAISTRVEEFITNVQRYDEFVKADAAL |
| | | PSRMLVYGKPGTGKTMLSKYIATRLDFPLLTVRCDTLISSLLGQTSKNLRQVFDYVMQRPSV |
| | | LFLDEFDALAGARGNERDIGELQRVVTSLLQNMDAASEDTVIIASTNHEQLLDPAIWRRFSF |
| | | RIPMPLPDIHQRELIWKNRLKNMICSDLDLSDLSRKSEGLSGAIIEQVSLDARRDAVIEGAS |
| | | VINHHKLYRRLYLAQSLMEGVNLSTYEDEIRWLRSKDKKLFSIRVLANLYKLTSRVISNILK |
| | | ESGAYEQKGYTV* (SEQ ID NO: 202) |
| | B | MSRRGTQFSNAKVTNPMLRIPFSSSDLGAIVNAGGGAKVLVDVTAEYRQGLVRNLTTSKHYL |
| | | ESKLSEYPGSLGTLVFKLRDQGIAKTHRPNKIAQEAGLQNAGHAKIDEMLVAAHAGCFDVLE |
| | | SVILHRNIKAILANLSAERIEPWDENRKVPGGTDGLFESSNILVRLFEYTGEDATYNNYENV |
| | | ISILEQHGVKYDEIRQKCGLPLLRIMDLSPNDRYILDILIDYPGIRTLIPEPKYSAFPVSVS |
| | | DSVGIETNSFPVPSEELPIVAVFDTGVSPIAATITPWVVSRETYVIPPDTSYEHGTMVSSLI |
| | | SGAHFLNDNHPWIPDTKSKIHDVCALDENGSYISDLILRLADAVNKRPDIKVWNLSLGGGPC |
| | | NEQTFSDFAMELDRLSDKFGILFVVAAGNYVDEPIRTWPNPDPLGGADLISSPGESVRALTV |
| | | GSVSHMEANDALSEIGTPTPYTRRGPGPVFTPKPDIIHAGGGVHRPWNVGASSLKVVGPDNR |
| | | LCSNFGTSFAAPIVASLAAHTWQRIATNTDFNVSPSLIKALLIHSAQLSSPDYSPSERRYLG |
| | | AGIPNEVIETLYDSDDRFTLIFQTFLVPGVRWRKDNYPIPSALIQNGKFKGEIVITAAYAPP |
| | | LNPNAGSEYVRANVELSFGLIENNTIKGKVPMEGENGQSGYERAQIEHGGKWSPVKIHRKAF |
| | | NKGITSGNWALQAKTTLRANEPALMEPLPVTIVVTLKSLDGNTQVYADGVRALNANNWAHYP |
| | | LPARVPVSV* (SEQ ID NO: 203) |
| 27 | A | MKTVRSACQLQPKALEINVGDQIEQLDQIINDTNGQEYFKKTFITDGFKTLLSKGMARLAGK |
| | | SNDTVFHLKQAMGGGKTHLMVGFGLLAKDAALRNSHLGSMPYQSDFGSAKIAAFNGRNNPHS |
| | | YFWGEIARQLGREGVFREYWESGAKAPDEQAWINIFDGEEPILILLDEMPPYFHYYSTQVLG |
| | | QGTIADVVTRAFSNMLTAAQKKKNVCIVVSDLEAAYDTGGKLIQRALDDATQELGRAEVSIT |
| | | PVNLESNEIYEILRKRLFLSLPDKNEVSEIASIYASRLAEAAKAKTVERSAEALANDIESTY |
| | | PFHPSFKSIVALFKENEKFKQTRGLMELVSRLLKSVWESDEEVYLIGAQHFDLSIHDVREKL |
| | | AEISEMRDVIARDLWDSTDSAHAQIIDLNNGNHYAQQVGTLLLTASLSTAVNSVKGLTESEM |
| | | LECLIDPNHQGSDYRNAFTELAKSAWYLHQTQEGRNYFSHQENLTKKLQGYADKAPQNKVDE |
| | | LIRHRLEEMYRPVTKEAYEKVLPLPEMDEAQATLRSGRALLIISPDGKTPPGVVGNFFKGLV |
| | | NKNNILVLTGDKSSIASIEKAARHVYAVTKADNEITASHPQRKELDEKKAQYEQDFQTTVLS |
| | | VFDKLLFPGNNRGEDVLRPKALDSTYPSNEPYNGERQVVKTLTSDPIKLYTQINENFDALRA |
| | | RAESLLFGTLDEARKTDLLDKMKQKTQMPWLPSRGFDQLAIEAYQRGVWEDLGNGYITKKPK |
| | | PKTTEVIISEDSSPDDAGTVRLKIGVANAGNSPRIHYAEDDEVTESSPVLSDNTLATKALRV |
| | | QFLAVDPTGKNLTGNPTTWKNRLTLRNRFDEVARTVELFVAPRGTIKYTLDGSEARNGETYT |
| | | VPIQLADQEATIYVFAECDGLEEKRNFTFAAAGSKEIPIIKDKPATLVSPSPKRMDSSAKTY |
| | | EGLKIAKEKGIEFEQISLMVGSAPKVIHISLGEMKISAEFIETVLTHLQTVLSPEAPVVMTF |
| | | KKAYTQTGHDLEQFVKQLGIEIGNGEVEQR* (SEQ ID NO: 204) |
| | B | MNKTVDFGAPSEFGMHHFYVEIPAAPRDAVVIYEDYGFDGEDSRRETVECRLILARELWTKI |
| | | RDDVRRDFNARLKIKKQSSGTWSTGKVKLDRFLGRELCVLGWAAEHASPDECLVICQKWLAL |
| | | RPEERWWLYSKTAAEAGRDDQTQRGWRKALYCALSDGANIKLETKKKPKSKKLQVEDETQDL |
| | | FGFMEKGEF* (SEQ ID NO: 205) |
| | C | MALQPFEWRDKPSLIEHLFPVQKISAETFKERMASHGQLLVSLGAFWKGRKPLHNKACILGS |
| | | LLPATDNPLEDLEVFELLMGIDSESMQKRIEASLPASKQETIGDYLVLPYAEQIRIAKRPEE |
| | | IDESLFVHIWNRVNNHLGTSAHTFAQLVEELGVARFGHRPRVADVFSGSGQIPFEAARLGCD |
| | | VYASDLNPISCMLTWGALNVVGASAQKRVEIDKAQRDIVKKVQKEIDELDIESDGRGWRAKV |
| | | FLYCVEVTCPESGWRVPLIPSLIISNSFRVVAELKPVPAERRYDISIREVSTDEELEFYKSG |
| | | TIQDGEVIHSPDGKTQYRVNDCTIRGDYKEGKENLNKLRMWEKTDFAPRPDDIFQDRLFCVQ |
| | | WMKKKPKGSQYYYEFRTVTNDDLKREKKVIEHVASKLDDWQKQGLVPDMVIEAGDKTDEPIR |
| | | TRGWTHWHHLFHPRQLLFLSLVNKYSLAEGKFNFLQCMNFILSKLTRWRPQAGGGGGSAATF |
| | | DNQALNTLYNYPVRATGSIENILAAQHNHCGISENVSFVVNSHPAPELDVENDIYITDPPYG |
| | | DAVKYEEITEFFIAWLRKNPPKEFAHWTWDSRRSLAVKGEDEGFRTGMVAAYRKMAQKMPDN |
| | | GLQVLMFTHQSGAIWADMANIIWASGLQVTAAWYVVTETDSALRGGSNVKGTIILILRKRHQ |
| | | ALETFRDDLGWEIEEAVKEQVESLIGLDKKVRSQGAEGLYTDADLQMAGYAAALKVLTAYSR |
| | | IDGKDMVTEAEAPRQKGKKTFVDELIDFAVQTAVQFLVPVGFEKSEWQKLQAVERFYLKMAE |
| | | MEHQGAKTLDNYQNFAKAFKVHHFDQLMSDASKANSARLKLSTEFRSTMMSGDAEMTGTPLR |
| | | ALLYALFEISKEVEVDDVLLHLMENCPNYLPNKQLLAKMADYLAEKREGLKGTKTFNPEQEA |
| | | SSARVLAEAIRNQRL* (SEQ ID NO: 206) |
| | D | MAIKRFSSRTERLDTEFLAESLKGAAKYFRIAGYFRSSIFELVGEEIAKIPEVKIICNSELD |
| | | LADFQVATGRNTALKERWNEVDVEAEALLKKERYQILDQLLHSGNVEIRWPRERLFLFIGKA |
| | | GSIHYADGSRKSFIGSVNESKSAFAHNYELVWQDDDEESADWVEREFWALWTEGVPLPDAIL |
| | | AEIHRVSNRREVTVDVLKPEEVPAAAMAEAPIYRGGEQLQPWQRSFVTMFLEHREIYGKARL |
| | | LLADEVGVGKTLSMATSALVSALLDDGPVLILAPSTLTIQWQIEMMDKLGVPAAVWSSQKKV |
| | | WLGVEGQILSPRGDASSIKKCPYRIAIISTGLIMHQREKTDFVKEAGMLLKNRFGTVILDEA |
| | | HKARIRGGLGDQASEPNNLMAFMLQIGRRTRHLVLGTATPIQTNVRELWDLLGILNSGAEFV |
| | | LGDALSPWHDHEQAIPLITGQTQVTSEAEVWHWLSNPLPPSNEHHTVQQIRDYLSIDNKSFG |
| | | YSHRFEDLDYMIQSLWLSECMTPSFFKENNPILRHTVLRKRKQLEDDGLLERVGVNTHPIKR |
| | | NLAQYQSRFVGLGIPTNTPFQVAYEKAEEFSKLLQSRTRAAGFMKSLMLQRICSSFASGLKT |
| | | AQKMLKHTVSDEDEDLVEDVEHLLSEMTPAEVACLREIETQLSRPEAVDSKLNTVKWFLTEF |
| | | RTDGKTWLEHGCIIFSQYYDTAEWTAKELAKSLKGEVVAVYAGVGKSGLFRGEQFNNVEREL |
| | | IKSAVKTREILLVVATDAACEGLNLQTLGTLINVDLPWNPSRLEQRLGRIKRFGQTRKFVDM |
| | | LNLWSETQDEKVYNVLSERLRDTYDIFGSLPDTIDDEWIDNEEELNTRMDEYMHERKKAQDA |
| | | FSVKYRGTLDPDAHLWERCATVLSRRDIVSKLSEPWGS* (SEQ ID NO: 207) |
| 28 | A | MSEQFVSEAAGTPHLAEQDDGLKNLKLLEESFNTDKLNSSEQKKLQELRSILSPLLKKGGVL |
| | | ADLFQDGKDVLAFPIDVDSVLQHLNQDMRDDWFTDTLQHKDLLSNKQSLHEVLHELLNEGNG |
| | | QYIGSFRSVYNIPKKGLGIRYSLETDFYDRFIYQAICTFLIQFYDPLLSHRVLSHRFNKDRK |
| | | SEKYIFKSRIDLWQTFEGVTRTALSNNQSLLATDLINCYENITIETIRTAFERSIEHINTSG |
| | | PNKVLIRNAVQTLCNLLSRWGYSERHGLPQNRDASSFIANWLNDIDHEMVRLGYDYYRYVDD |
| | | IRYICPNTRVAKKALTELINQLRKVGMNINSGKTKILTQDSTANEVDEFFPTSDDRSLTIDN |
| | | MWRSRSRRVIARSAKYIFQELKECIEEKQTQSRQFRFAVNRLIKLTDAGIFDIHATIATDLK |
| | | ALLISSLEDHAASTDQYCRLLGILDLNEHELNDIYNHLSDHERSVHSWQNFHLWLLLANRKY |
| | | KSTNLITLATARIESDILQPEIAAIFIYLKCVGEAQVLIDNISKFESAWPYYHQRNFLLACS |
| | | DFDHNQLKPLISKLGPKLKWTGSRAKPYFTNGMPLVERDKIAMLDLYDEITPYD* |
| | | (SEQ ID NO: 208) |
| | B | MTESKKALLFIADYTDQGQDRIFLWSDGTLGEVTISDLVDQKHELVCHDLWLIAPSLYRATN |
| | | KLPSNITDIEELRILTSGKKKERESRDKKDISQLLSSFVSEETIARYKEIFNRKIPLDEAVL |
| | | SSIGEALLKCSEWKSDANTAGEWERFITERPVNDYLIRSTSEGISISEEKLRYHKNKIEFEF |
| | | YMALKSFSSDYDMPLEVPSDQAVIEYLEPKGFDFTGLDVDYILNFVPMQSHFAEDLIRLRKI |
| | | QNSRRVLAAIPLSQSRIYPIVDSFGSITSRIYFKDPSLQNLAKHHRDILIPDTNKQLSYIDY |
| | | DQFEAGVMAALSGDEKLLELYNSSDVYEIAAKEIFDDKSKRKQAKRLFLSYAYGMKRQHILA |
| | | AAQGFGADRQNAKKFFEQFKTFEAWKVLVHEEFHRTGRIGTALGNYMHRERKGELTSKEKRS |
| | | AISQIVQGTASLIFKKALLCLSSISEVKLKLPMHDAVLLEHPADYDMDRVINIFSEIMSEHF |
| | | QNKIQGKASLSQFHEDL* (SEQ ID NO: 209) |
| 29 | A | MSVIRGLAAVLRQSDSDISAFLVTAPRKYKVYKIPKRTTGFRVIAQPAKGLKDIQRAFVQLY |
| | | SLPVHDASMAYMKGKGIRDNAAAHAGNQYLLKADLEDFFNSITPAIFWRCIEMSSAQTPQFE |
| | | PQDKLFIEKILFWQPIKRRKTKLILSVGAPSSPVISNFCMYEFDNRIHAACKKVEITYTRYA |
| | | DDLTFSSNIPDVLKAVPSTLEVLLKDLFGSALRLNHSKTVFSSKAHNRHVTGITINNEETLS |
| | | LGRDRKRFIKHLINQYKYGLLDNEDKAYLIGLLAFASHIEPSFITRMNEKYSLELMERLRGQ |
| | | R* (SEQ ID NO: 210) |
| | B | MTKQYERKAKGGNLLSAFELYQRNSDKAPGLGEMLVGEWFEMCRDYIQDGHVDESGIFRPDN |
| | | AFYLRRLTLKDFRRFSLLEIKLEEDLTVIIGNNGKGKTSILYAIAKTLSWFVANILKEGGSG |
| | | QRLSEMTDIKNDAEDRYSDVSSTFFFGKGLKSVPIRLSRSALGTAERRDSEVKPAKDLADIW |
| | | RVINEVNTINLPTFALYNVERSQPFNRNIKDNTGRREERFDAYSQTLGGAGRFDHFVEWYIY |
| | | LHKRTVSDISSSIKELEQQVNDLQRTVDGGMVSVKSLLEQMKFKLSEAIERNDAAVSSRVLT |
| | | ESVQKSIVEKAICSVVPSISNIWVEMITGSDLVKVTNDGHDVTIDQLSDGQRVFLSLVADLA |
| | | RRMVMLNPLLENPLEGRGIVLIDETELHLHPKWQQEVILNLRSAFPNIQFITTTHSPIVLST |
| | | IEKRCIREFEPNDDGDQSFLDSPDMQTKGSENAQILEQVMNVHSTPPGIAESHWLGNFELLL |
| | | LDNSGELDNHSQVLYDQIKAHFGIDSIELKKADSLIRINKMKNKLNKIRAEKGK* |
| | | (SEQ ID NO: 211) |
| | C | MRELARLERPEILDQYIAGQNDWMEIDQSAVWPKLTEMQGGFCAYCECRLNRCHIEHFRPRG |
| | | KFPALTFIWNNLFGSCGDSRKSGGWSRCGIYKDNGAGAYNADDLIKPDEENPDDYLLFLTTG |
| | | EVVPAIGLTGRALKKAQETIRVFNLNGDIKLFGSRRTAVQAIMPNVEYLYTLLEEFDEDDWN |
| | | EMLRDELEKIESDEYKTALKHAWTFNQEFA* (SEQ ID NO: 212) |

Sequence of vector backbone. Inserts were cloned between the HindIII and EcoRI restriction sites (underlined).

(SEQ ID NO: 213)
CCCGCGCCCACCGGAAGGAGCTGACTGGGTTGAAGGCTCTCAAGGGCATC
GGTCGAGATCCCGGTGCCTAATGAGTGAGCTAACTTACATTAATTGCGTT
GCGCAAGCTTCTGCAGAATTCGCAATAACTAGCATAACCCCTTGGGGCCT
CTAAACGGGTCTTGAGGGGTTTTTTGCTGAAACCTCAGGCATTTGAGAAG
CACACGGTCACACTGCTTCCGGTAGTCAATAAACCGGTAAACCAGCAATA
GACATAAGCGGCTATTTAACGACCCTGCCCTGAACCGACGACCGGGTCGA
ATTTGCTTTCGAATTTCTGCCATTCATCCGCTTATTATCACTTATTCAGG
CGTAGCACCAGGCGTTTAAGGGCACCAATAACTGCCTTAAAAAAATTACG
CCCCGCCCTGCCACTCATCGCAATACTGTTGTAATTCATTTAACATTCTG
CCGACATGGAAGCCATCACAGACGGCATGATGAACCTGAATCGCCAGCGG
CATCAGCACCTTGTCGCCTTGCGTATAATATTTGCCCATAGTGAAAACGG
GGGCGAAGAAGTTGTCCATATTGGCCACGTTTAAATCAAAACTGGTGAAA
CTCACCCAGGGATTGGCTGAGACGAAAAACATATTCTCAATAAACCCTTT
AGGGAAATAGGCCAGGTTTTCACCGTAACACGCCACATCTTGCGAATATA
TGTGTAGAAACTGCCGGAAATCGTCGTGGTATTCACTCCAGAGCGATGAA
AACGTTTCAGTTTGCTCATGGAAAACGGTGTAACAAGGGTGAACACTATC
CCATATCACCAGCTCACCGTCTTTCATCGCCATACGGAACTCTGGATGAG
CATTCATCAGGCGGGCAAGAATGTGAATAAAGGCCGGATAAAACTTGTGC
TTATTTTTCTTTACGGTCTTTAAAAAGGCCGTAATATCCAGCTGAACGGT
CTGGTTATAGGTACATTGAGCAACTGACTGAAATGCCTCAAAATGTTCTT
TACGATGCCATTGGGATATATCAACGGTGGTATATCCAGTGATTTTTTTC
TCCATTTTAGCTTCCTTAGCTCCTGAAAATCTCGATAACTCAAAAAATAC
GCCCGGTAGTGATCTTATTTCATTATGGTGAAAGTTGGAACCTCTTACGT
GCCGATCAACGTCTCATTTTCGCCAAAAGTTGGCCCAGGGCTTCCCGGTA
TCAACAGGGACACCAGGATTTATTTATTCTGCGAAGTGATCTTCCGTCAC
AGGTATTTATTCGGCGCAAAGTGCGTCGGGTGATGCTGCCAACTTACTGA
TTTAGTGTATGATGGTGTTTTTGAGGTGCTCCAGTGGCTTCTGTTTCTAT
CAGCTGTCCCTCCTGTTCAGCTACTGACGGGGTGGTGCGTAACGGCAAAA
GCACCGCCGGACATCAGCGCTAGCGGAGTGTATACTGGCTTACTATGTTG
GCACTGATGAGGGTGTCAGTGAAGTGCTTCATGTGGCAGGAGAAAAAAGG
CTGCACCGGTGCGTCAGCAGAATATGTGATACAGGATATATTCCGCTTCC
TCGCTCACTGACTCGCTACGCTCGGTCGTTCGACTGCGGCGAGCGGAAAT
GGCTTACGAACGGGGCGGAGATTTCCTGGAAGATGCCAGGAAGATACTTA
ACAGGGAAGTGAGAGGGCCGCGGCAAAGCCGTTTTTCCATAGGCTCCGCC
CCCCTGACAAGCATCACGAAATCTGACGCTCAAATCAGTGGTGGCGAAAC
CCGACAGGACTATAAAGATACCAGGCGTTTCCCCCTGGCGGCTCCCTCGT
GCGCTCTCCTGTTCCTGCCTTTCGGTTTACCGGTGTCATTCCGCTGTTAT
GGCCGCGTTTGTCTCATTCCACGCCTGACACTCAGTTCCGGGTAGGCAGT
TCGCTCCAAGCTGGACTGTATGCACGAACCCCCCGTTCAGTCCGACCGCT
GCGCCTTATCCGGTAACTATCGTCTTGAGTCCAACCCGGAAAGACATGCA
AAAGCACCACTGGCAGCAGCCACTGGTAATTGATTTAGAGGAGTTAGTCT
TGAAGTCATGCGCCGGTTAAGGCTAAACTGAAAGGACAAGTTTTGGTGAC
TGCGCTCCTCCAAGCCAGTTACCTCGGTTCAAAGAGTTGGTAGCTCAGAG
AACCTTCGAAAAACCGCCCTGCAAGGCGGTTTTTTCGTTTTCAGAGCAAG
AGATTACGCGCAGACCAAAACGATCTCAAGAAGATCATCTTATTAATCAG
ATAAAATATTTCTAGATTTCAGTGCAATTTATCTCTTCAAATGTAGCACC
TGAAGTCAGCCCCATACGATATAAGTTGTAATTCTCATGTTAGTCATGC

### Example 3—Diverse Enzymatic Activities Mediate Antiviral Immunity in Prokaryotes

Bacteria and archaea are frequently attacked by viruses and other mobile genetic elements and rely on dedicated antiviral defense systems, such as restriction endonucleases and CRISPR, to survive. The enormous diversity of viruses suggests that more types of defense systems exist than are currently known. By systematic defense gene prediction and heterologous reconstitution, here Applicants discovered 29 widespread antiviral gene cassettes, collectively present in 32% of all sequenced bacterial and archaeal genomes, which mediate protection against specific bacteriophages. These systems incorporate enzymatic activities not previously implicated in antiviral defense, including RNA editing and retron msDNA synthesis. In addition, Applicants found a diverse set of other defense genes. These results highlight an immense array of molecular functions that microbes employ against viruses.

Domain-independent identification of uncharacterized defense systems

Many antiviral defense genes in bacterial and archaeal genomes show a distinctive tendency to cluster together within defense ‘islands’ (7, 10). As a consequence, an uncharacterized gene whose homologs consistently occur next to, for instance, restriction-modification genes has an increased likelihood of being involved in defense (11, 12).

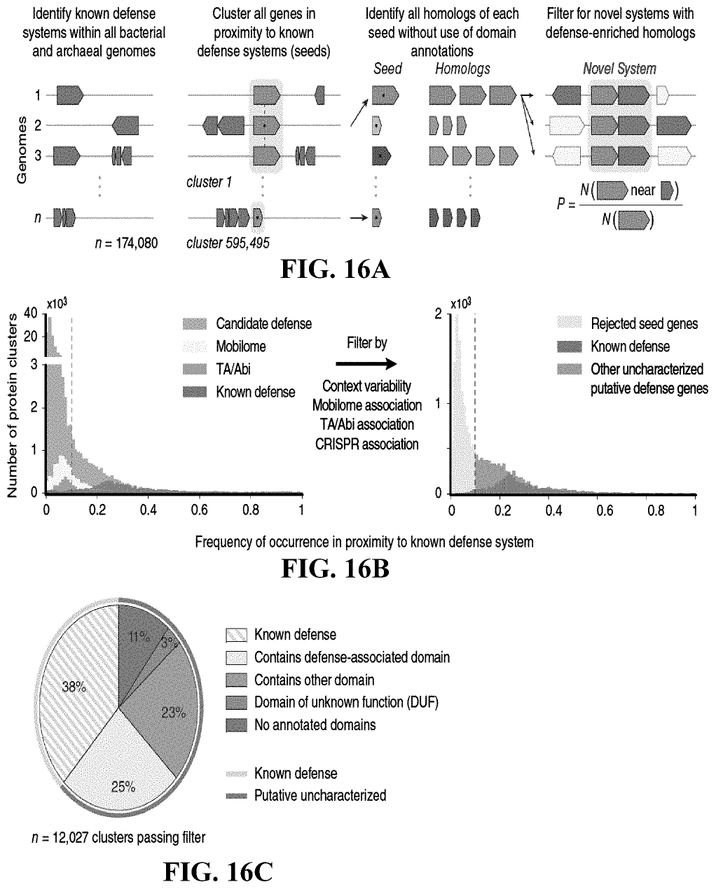

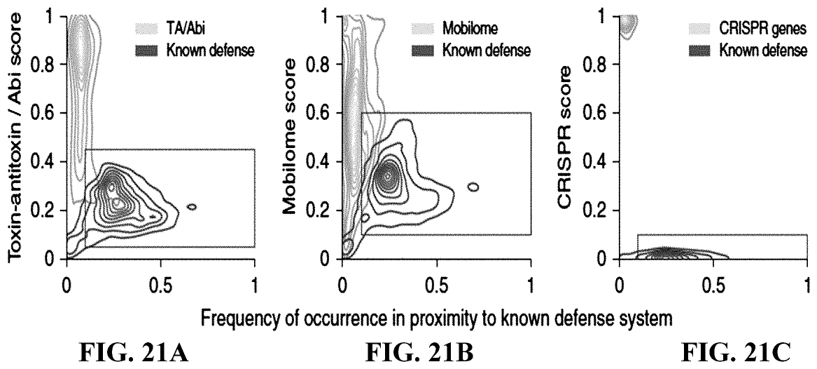

Applicants found that additional, unknown defense systems exist which either lack annotated domains, or only contain domains that are not typically associated with defense but have been co-opted in specific instances to perform defense functions. Applicants developed an expanded computational approach in which novel defense systems were identified independent of domain annotations (FIG. 16A). Applicants analyzed all bacterial and archaeal genomes available in Genbank as of November 2018, collectively encoding 620 million proteins. To identify candidate novel defense genes, Applicants first compiled a list of all genes within 10 kb or 10 open reading frames away from known defense systems (see Methods). This initial list (n=8.7×10⁶) which evidently contained both novel defense genes and non-defense ones, was clustered to yield 6×10⁵ representative sequences (“seeds”). To distinguish between defense and non-defense seeds, Applicants identified all homologs of each seed present in Genbank and analyzed their gene neighborhoods. The seed was predicted to be a defense gene if these neighborhoods resembled those of known defense gene—in particular, if a high percentage of homologs were located in proximity to known defense genes (“defense score”) and displayed context diversity (FIGS. 16B, 21A-21D, and Methods). All clustering and homolog detection steps were performed based on amino acid sequences, without invoking existing domain annotations and thus allowing the identification of novel types of defense genes.

After all filtering and curation steps, Applicants identified a total of 7,472 seeds that represented candidate defense genes, along with 4,555 seeds for known defense genes under the same analysis parameters (FIG. 16C). These seeds were analyzed with additional, more sensitive analysis of their domain content. Of the uncharacterized genes, 1,687 (23%) had either no annotated domains or contained only domains of unknown function (DUFs), and an additional 2,756 (37%) contained only domains that are different from the characteristic domains of known defense genes. These results suggested the existence of a diverse set of defense genes with mechanisms that remain to be investigated.

Candidate defense systems exhibit antiviral activity in a heterologous system

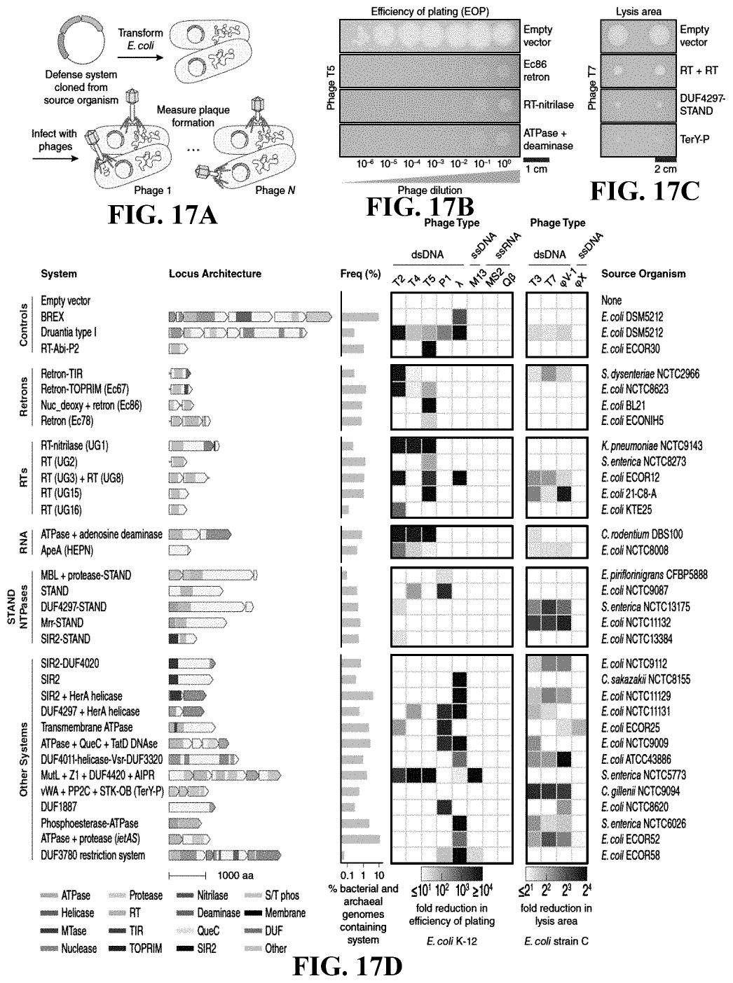

To characterize the functional diversity among the predicted defense genes, Applicants selected 48 candidate systems to test experimentally for defense activity. Candidate systems were prioritized based on the presence of predicted molecular functions not previously implicated in defense; broad phylogenetic distribution; the presence of at least one protein larger than 300 amino acids (to increase the likelihood of the presence of enzymes); and, for multi-gene systems, conservation of the component genes. Because wild-type bacterial strains are likely to harbor multiple active defense systems, thereby maintaining phage resistance even if one of the systems were knocked out (13), Applicants elected to assay activity by heterologous reconstitution. For each system, 1-4 homologs were selected, cloned from the source organism into the low-copy vector pACYC and transformed into Escherichia coli (FIG. 17A), comprising a total of 395 kb of exogenous DNA (see tables 9-16 for sequence, accession, and source organism information). Three previously identified defense systems, BREX type I (13, 14), Druantia type I (4), and the abortive infection reverse transcriptase RT-Abi-P2 (15) were included as positive controls. Each system was then challenged with a diverse panel of coliphages with dsDNA, ssDNA, or ssRNA genomes, and phage sensitivity of the bacteria was compared to that observed with the empty vector control (FIGS. 17B-17C).

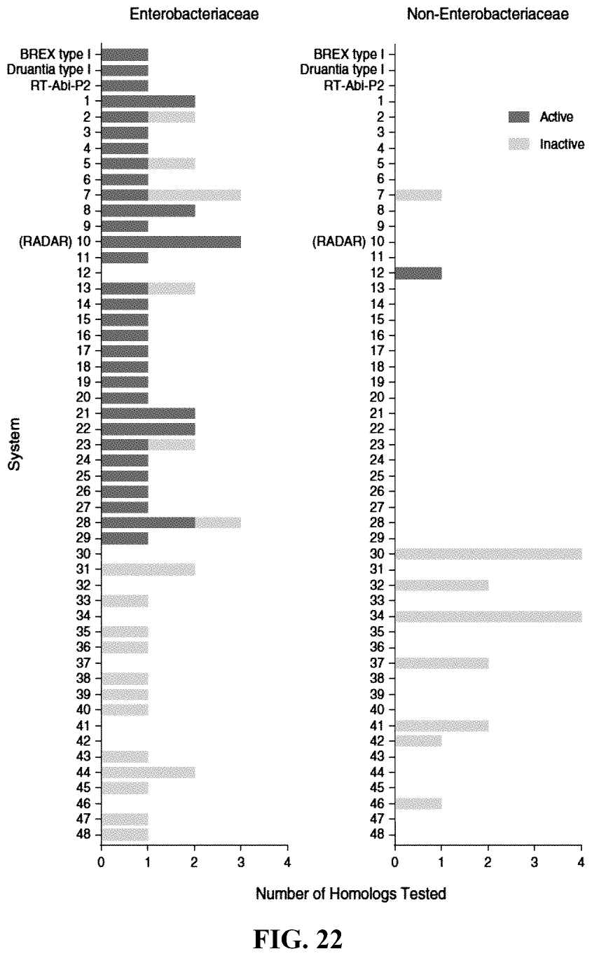

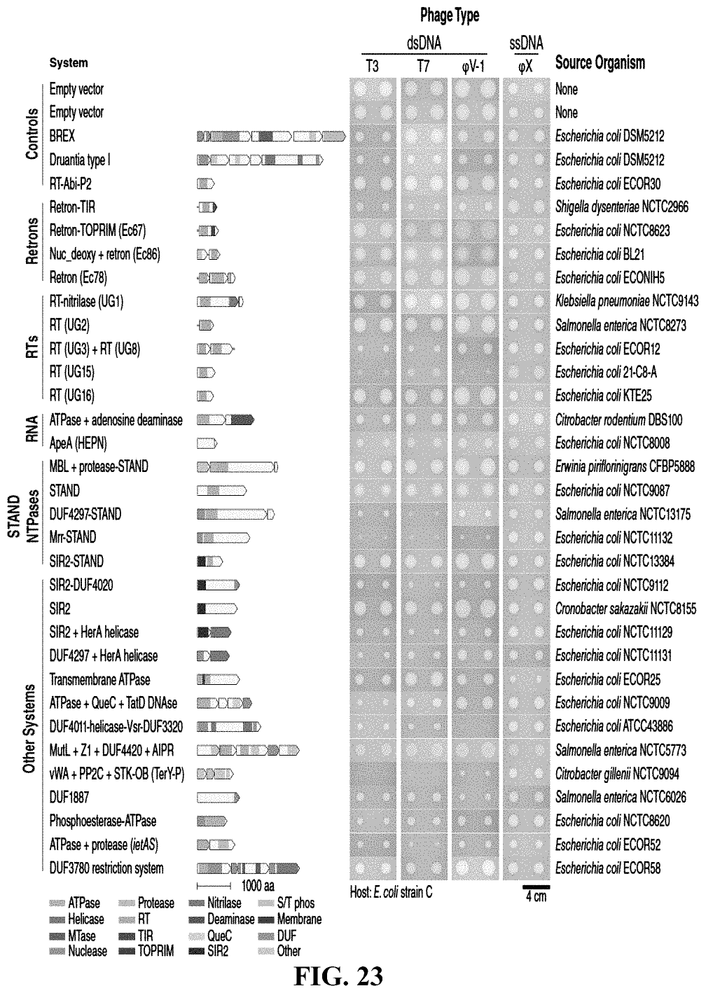

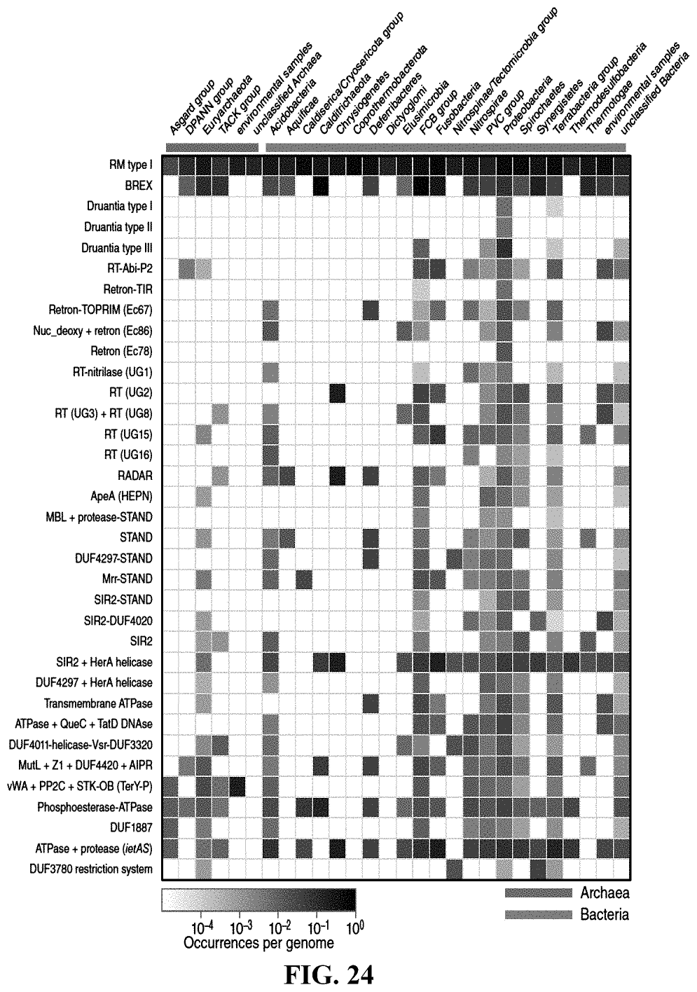

Applicants observed anti-phage activity for 29 of the 48 tested candidates (60%) (FIG. 22). Systems from source organisms outside the Enterobacteriaceae family, which had Escherichia and closely-related genera including Salmonella and Klebsiella, had little to no activity, suggesting the importance of host compatibility. The most active representative in each of these 29 systems (representing 4% of the uncharacterized defense seeds) was further tested with an expanded panel of phages in two E. coli strains (FIGS. 17D and 23). All 29 systems were active against at least one dsDNA phage, and four were active against ssDNA phages (M13 or φX174). Phage specificity was typically narrow and varied widely across systems. The abundance of these defense systems among the sequenced bacterial and archaeal genomes spans two orders of magnitude, ranging from ˜0.1% to ˜10% of the genomes (FIG. 17D). Overall, 32% of all sequenced bacterial and archaeal genomes contain at least one of these novel defense systems, which are broadly distributed across bacterial and archaeal phyla (FIG. 24).

RADAR with a divergent adenosine deaminase that edits RNA in response to phage infection

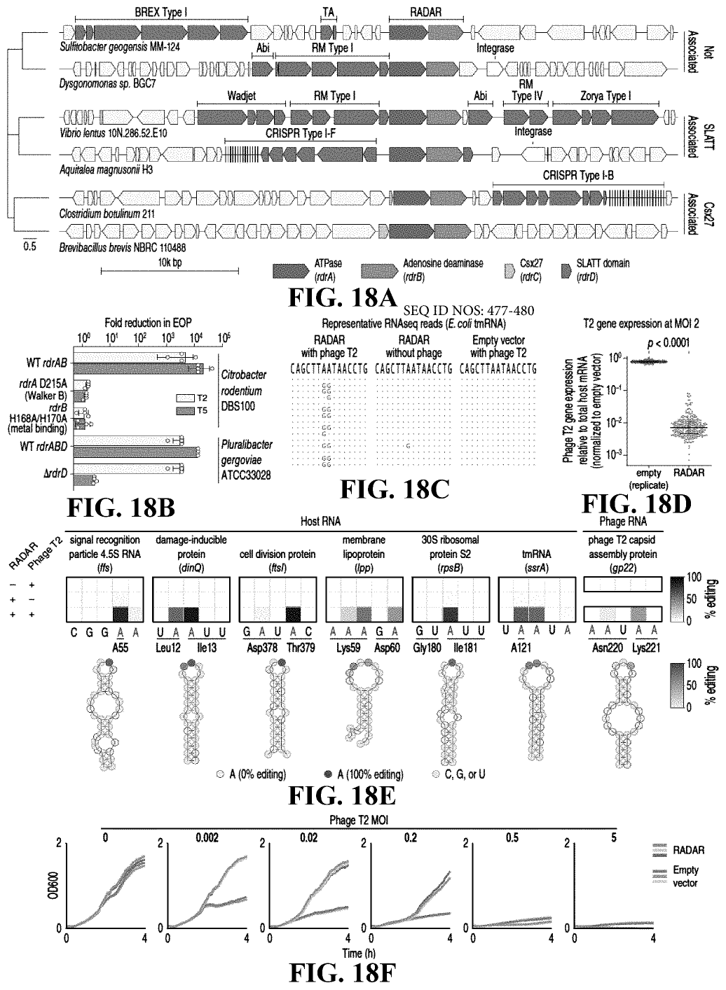

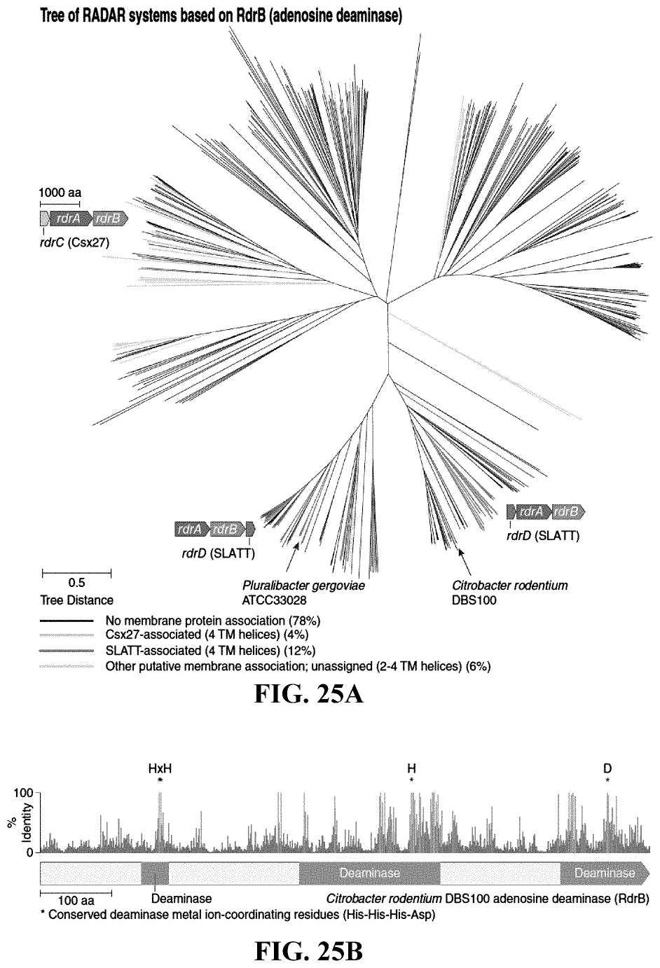

Applicants identified a two-gene cassette consisting of an ATPase (˜900 residues) and a divergent adenosine deaminase (˜900 residues) that was active against dsDNA phages T2, T3, T4, and T5. Because deaminase activity had not been previously implicated in antiviral defense, Applicants focused on this system for further investigation. The system appeared in diverse defense contexts and forms three subtypes (FIGS. 18A and 25A). In most cases, it had the ATPase and deaminase only, but some variants also included a small membrane protein, either a SLATT domain (16) or the type VI-B CRISPR ancillary protein Csx27 (17). Mutations in the ATPase Walker B motif or in the putative divalent metal cation-binding H×H motif of the deaminase abolished defense activity, whereas the SLATT domain membrane protein was required for resistance against phage T5 but not against phage T2 (FIG. 18B).

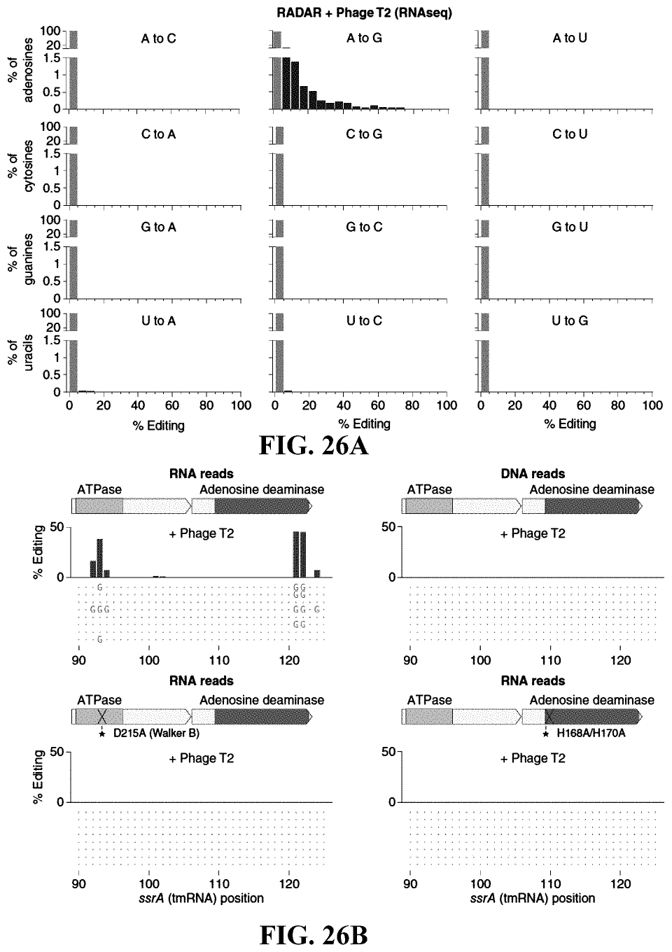

Given the large size of the deaminase compared to typical metabolic adenosine deaminases and its sequence divergence due to large insertions within the deaminase domain (FIG. 25B), Applicants found that it acted on nucleic acids rather than on free nucleosides or nucleotides. Applicants performed whole-transcriptome sequencing and found an enrichment of A to G substitutions in sequencing reads at specific sites in the presence of phage, whereas C, G, or U bases were not affected (FIGS. 18C and 26A), consistent with RNA editing of adenosine to inosine. Furthermore, the overall expression of phage genes, including early genes, was reduced by ˜100-fold even at a multiplicity of infection (MOI) of 2 (FIG. 18D). Since most of the cells in the culture were expected to be infected, this suggested that defense activity occurs early in the infection cycle, which was not evident from efficiency of plating (EOP) alone.

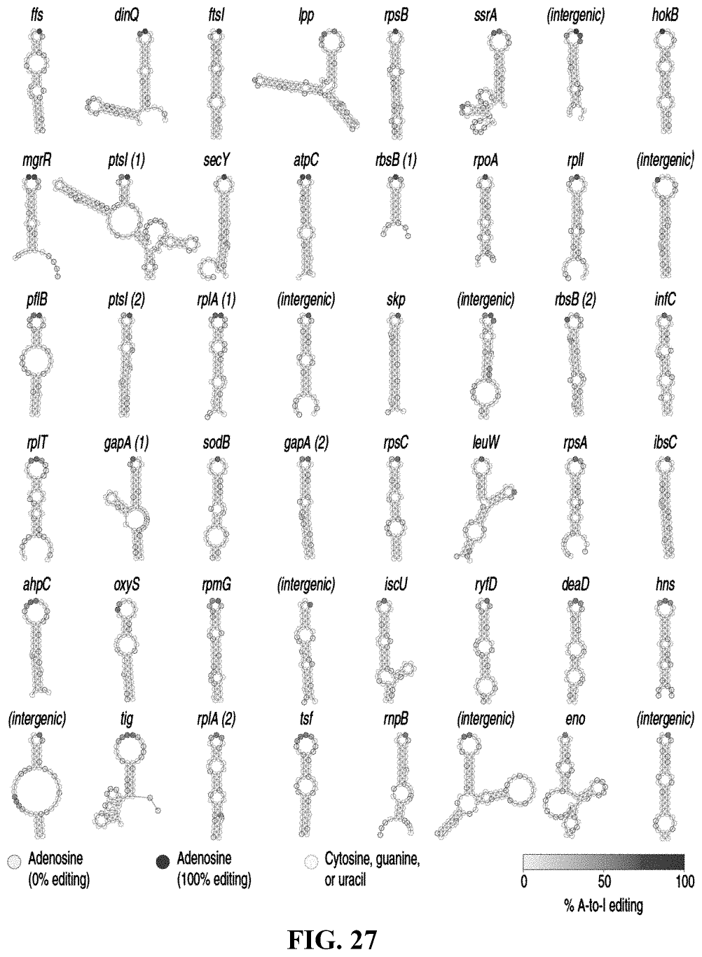

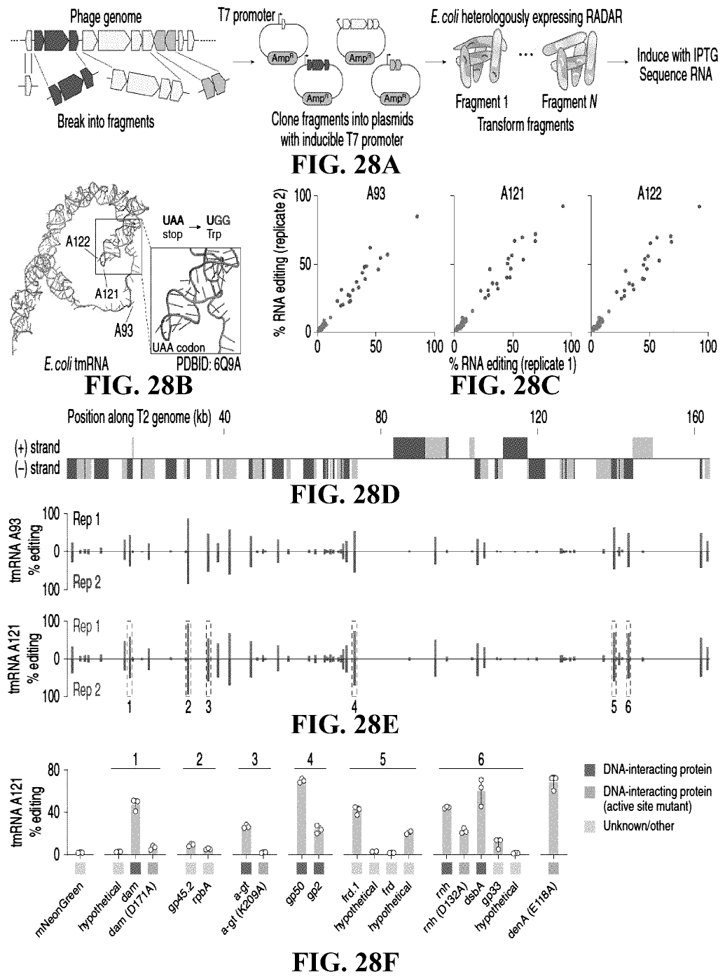

RNA editing occurred only when both the defense system and the phage were present; expression of the defense system without the phage resulted in a near-baseline level of editing, and no editing was detected in the absence of the system. Mutations in the ATPase or deaminase active sites abolished editing, and no DNA editing was detected (FIG. 26B). Editing sites were broadly distributed throughout the E. coli transcriptome (FIGS. 18E, 26A, 27, and table 17), and editing could also be induced by co-expressing specific phage proteins with the system (FIGS. 28A-28F and table 18). RNA secondary structure predictions indicated a characteristic stem-loop structure at strong editing sites; specific adenosines in loops were edited with up to ˜90% frequency, whereas adenosines within the stem were not edited within the limit of detection (FIGS. 18E and 27). Finally, some of the editing sites were deleterious to the host cell, resulting in nonsynonymous mutations such as at the UAA stop codon of the transfer messenger RNA (tmRNA) (FIG. 28B), which rescues ribosomes stalled during translation (18).

Based on these results, Applicants named this system phage restriction by an adenosine deaminase acting on RNA (RADAR). Growth kinetics at varying phage multiplicity of infection (MOI) revealed a threshold MOI above which RADAR-expressing cells had a lower OD600 compared to the empty vector control, suggestive of RADAR-mediated growth arrest (FIG. 18F). Together with the abundance and broad distribution of editing sites in the host transcriptome (FIGS. 26A-26B, 27), these results are consistent with an editing-dependent abortive infection mechanism that is activated by phage.

A widespread family of defense systems containing reverse transcriptases

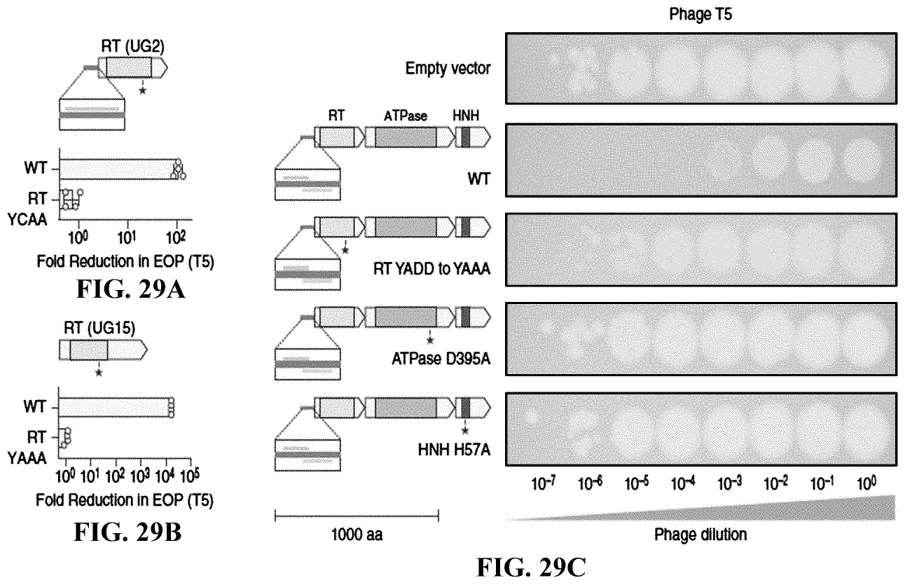

Applicants discovered that a family of uncharacterized reverse transcriptases (RTs) are active defense systems. Although most RTs in prokaryotes are components of mobile retroelements, distinct clades of RTs that lack the hallmarks of mobility also exist, including 16 ‘unknown groups’ (UGs) (19-22). Applicants independently identified many of these uncharacterized RTs via the pipeline, suggesting that they might be defense genes (FIG. 19A). Indeed, six of these candidates (UG1, UG2, UG3, UG8, UG15, and UG16) provided robust protection against dsDNA phages. In all cases, mutations in the RT active site ((Y/F)×DD (SEQ ID NOS: 1-2) to (Y/F)×AA) abolished activity (FIGS. 19B and 29A-29B). Applicants named these genes defense-associated RTs (DRTs).

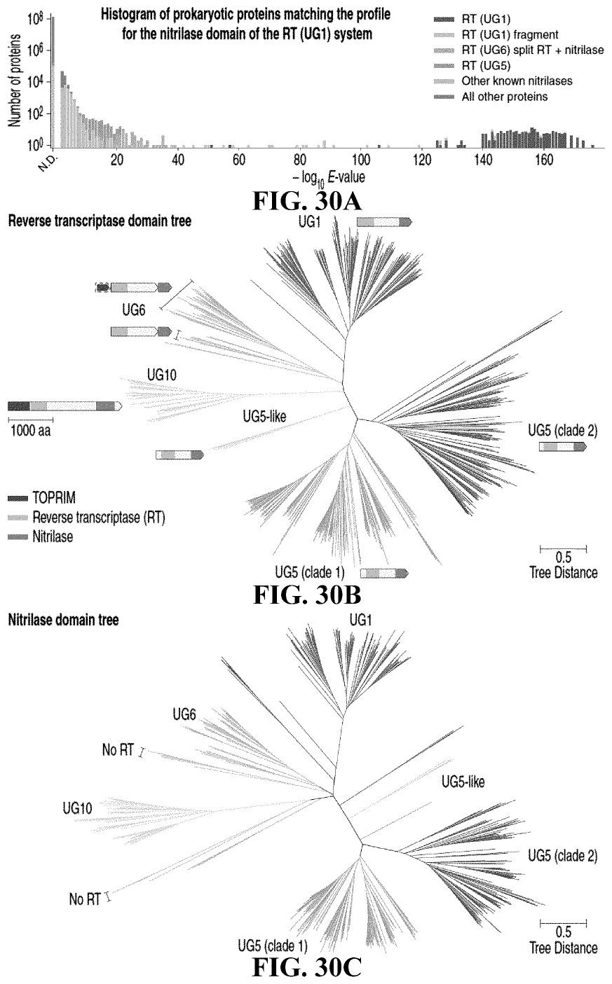

Each of these RT systems displayed a distinct pattern of phage resistance (FIG. 17D). Moreover, while UG2 (drt2), UG15 (drt4), and UG16 (drt5) act as individual genes, the UG3 (drt3a) and UG8 (drt3b) RTs were components of the same defense system (DRT type 3), with both RTs required for defense activity. Like RADAR, some subtypes of the UG1 (DRT type 1) and DRT type 3 systems were also associated with small membrane proteins (FIG. 19A). Moreover, DRT type 1 encompassed a much larger protein (˜1200 residues) than the other five RTs and also contains a C-terminal nitrilase domain. Mutation of the catalytic cysteine of the nitrilase (C1119A) abolished the activity (FIG. 19B). Nitrilases typically function in processes unrelated to defense, such as nucleotide metabolism and small molecule biosynthesis (23). Thus, DRT type 1, which is divergent from typical nitrilases and forms a distinct clade in the phylogenetic tree of the nitrilase family (FIGS. 30A-30C), exemplifies a non-defense domain that was apparently co-opted for a defense function.

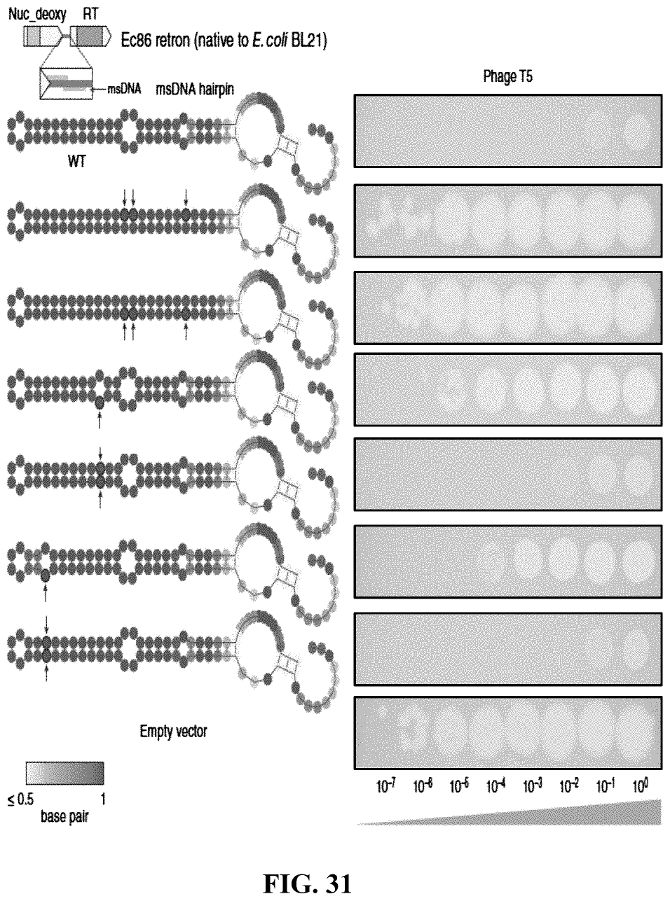

To further characterize these RTs, Applicants performed whole transcriptome sequencing of RT-expressing E. coli during phage infection. These experiments revealed substantial differences in phage gene expression across the different RTs (FIG. 19C). For instance, DRT type 1 strongly suppressed the expression of phage late genes, such as capsid proteins, whereas early and middle genes were not substantially affected, suggesting that it is active prior to the late stage of infection but does not prevent the injection of phage DNA into the host cell. In contrast, DRT type 3 did not strongly suppress expression of any of the phage genes, despite growing at a rate similar to DRT type 1 during phage infection (FIG. 31A). Transcriptome sequencing also identified a highly expressed, structured non-coding RNA at the 3′ end of the DRT type 3 system that is required for activity (FIGS. 19B, 19D-19E).

Retrons Mediate Anti-Phage Defense

Applicants also found that retrons, a distinct class of RTs that produce extrachromosomal satellite DNA (multi-copy single-stranded DNA, msDNA), are active anti-phage defense systems. The retron msDNA is produced from the 5′ UTR of its own mRNA and is covalently linked to an internal guanosine of the RNA via a 2′-5′ phosphodiester bond (24). First identified over 30 years ago, retrons have been harnessed for bacterial genome engineering (25), but their native biological function has remained unknown. Applicants found that the original E. coli retrons Ec67 (26) and Ec86 (27), as well as a homolog of the Ec78 retron (28) and a novel TIR (Toll/interleukin 1 receptor) domain-associated retron, mediate defense against dsDNA phages. Of note, the Ec86 retron is natively present in the widely-used laboratory E. coli strain BL21. Mutations in the (Y/F)×DD (SEQ ID NOS: 1-2) active site motif of the RT, as well as at the branching guanosine, abolished activity, indicating that the defense function depends on msDNA synthesis (FIGS. 19B and 29C). Furthermore, perturbations to the msDNA also abolished activity (FIG. 31), suggesting that its structure, and not simply formation, is essential for the defense activity. Indeed, a single nucleotide mismatch in the msDNA hairpin reduced activity by 100-1000 fold, but introducing a second mutation on the complementary strand to restore the structure of the msDNA also restored wild-type activity (FIG. 31). Notably, these retrons are associated with other domains, including TOPRIM (topoisomerase-primase) (29), TIR (30), a nucleoside deoxyribosyltransferase-like enzyme, and the Septu defense system (4), all of which play a role for activity (FIG. 19B).

Additional Molecular Functions of Defense Systems

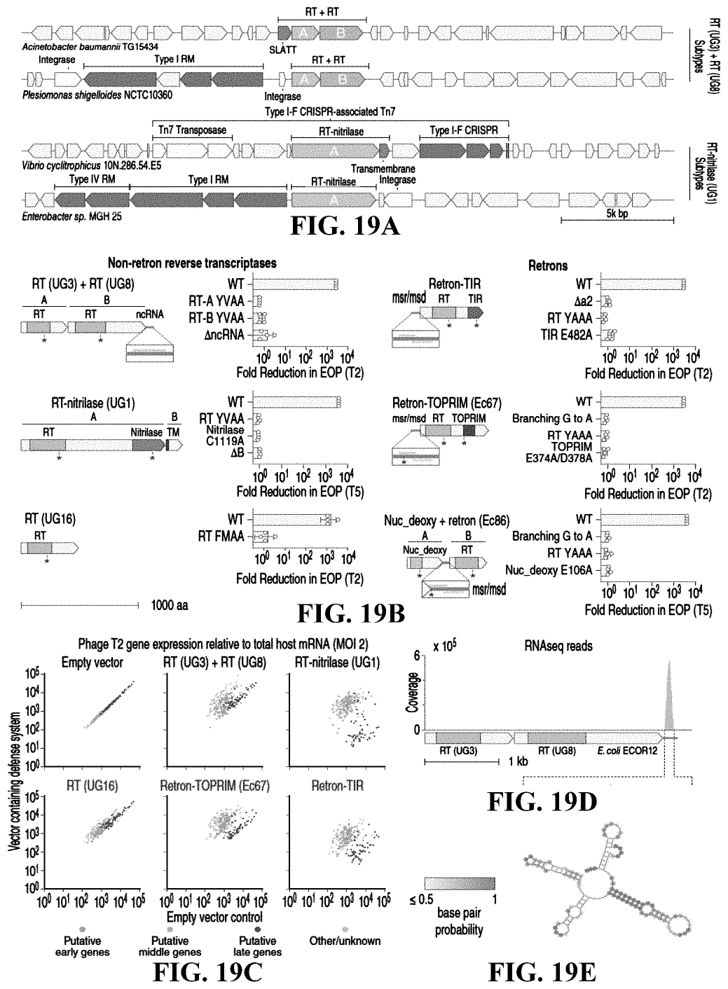

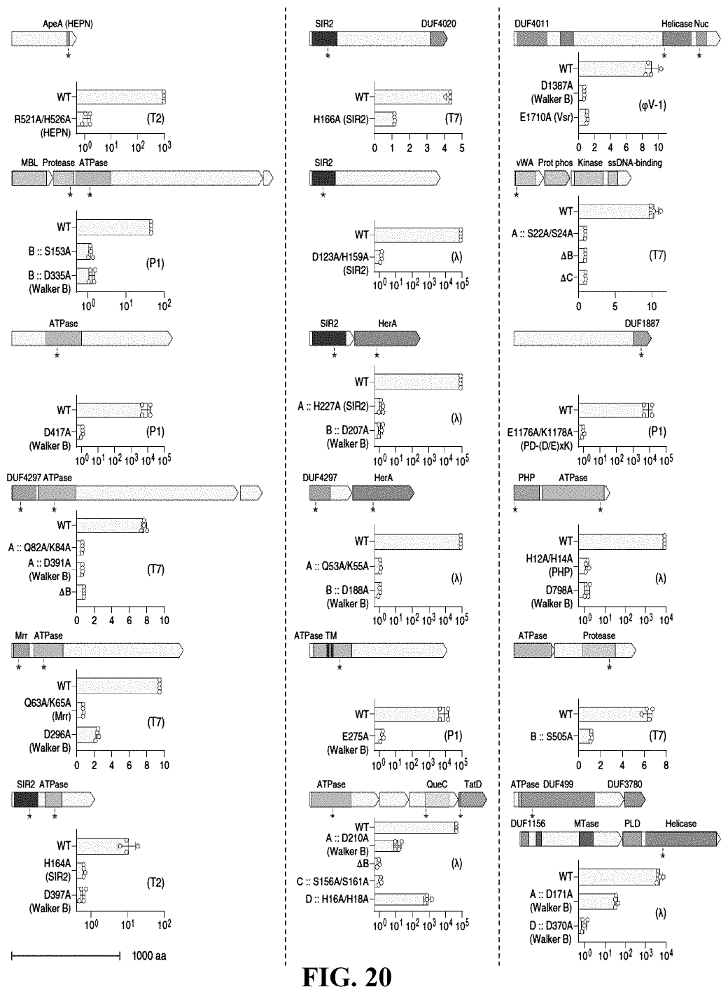

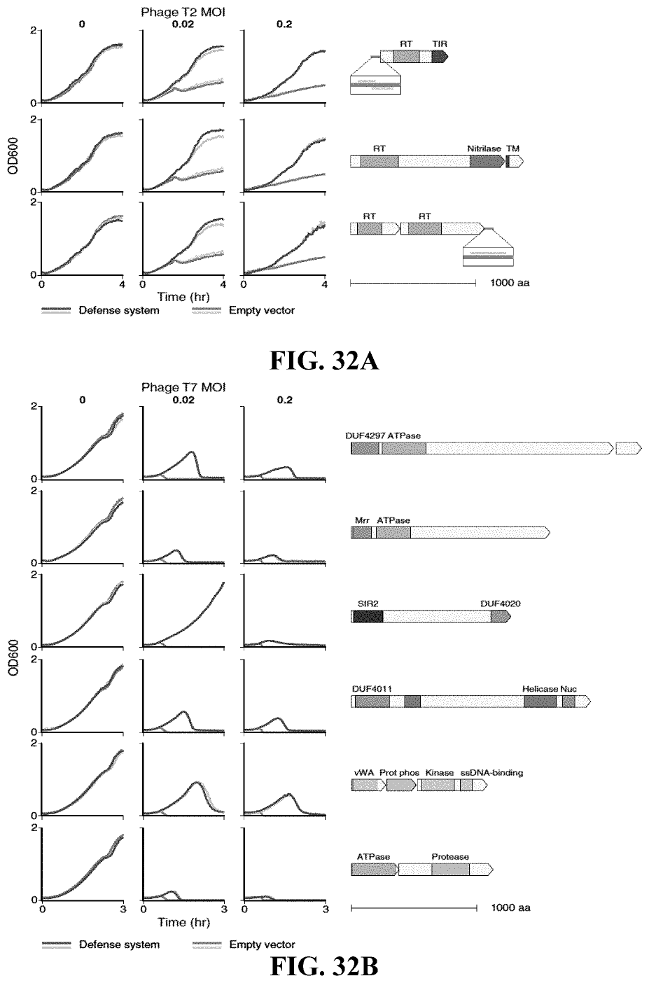

Applicants investigated several additional systems with diverse components (FIGS. 20, 32A-32B). These include a three-gene system containing a von Willebrand factor A (vWA) metal ion binding protein, a PP2C-like serine/threonine protein phosphatase, and a serine/threonine protein kinase that provided strong protection against T7-like phages (T3, T7, and φV-1). This system, dubbed TerY-phosphorylation triad (TerY-P), has been previously analyzed computationally in the context of tellurite resistance-associated stress response and might operate as a phosphorylation switch that couples the activities of the kinase and the phosphatase (31).

Additional systems include proteins containing a SIR2 (sirtuin) deacetylase domain that is also present in the recently-discovered Thoeris system (4) and has also been detected in the same neighborhoods with prokaryotic Argonaute proteins (32); ApeA, a predicted HEPN-family abortive infection protein (33) and a putative ancestor of the type VI CRISPR effector Cas13; a ˜1300 residue P-loop ATPase containing an unusual insertion of two transmembrane helices into the ATPase domain, similar to the KAP ATPases (34); and a four-gene cassette containing a 7-cyano-7-deazaguanine synthase-like protein (QueC), suggestive of small molecule biosynthesis. All of these components are essential for defense activity (FIG. 20).

Finally, Applicants also demonstrated defense functions for several predicted NTPases of the STAND (signal transduction ATPases with numerous associated domains) superfamily (FIG. 20). This expansive superfamily comprise multidomain proteins that include eukaryotic ATPases and GTPases involved in programmed cell death and various forms of signal transduction (35, 36). Typically, STAND NTPases contain a C-terminal helical sensor domain that, upon target recognition, induces oligomerization via ATP or GTP hydrolysis, leading to activation of the N-terminal effector domain. The role of the STAND NTPases in prokaryotes has long remained enigmatic (35, 37); the few for which experimental data are available contain a helix-turn-helix domain and have been shown to regulate transcription (36). Several STAND NTPases were active against dsDNA phages (FIG. 17D); these proteins contained different putative effector domains, including DUF4297 (a putative PD-(D/E)×K-family nuclease), an Mrr-like nuclease, SIR2, a trypsin-like serine protease, and an uncharacterized helical domain. Applicants named these systems antiviral ATPases/NTPases of the STAND superfamily (AVAST). As homologs of essential eukaryotic programmed cell death effectors, AVAST systems are likely to function via an abortive infection mechanism, i.e. by causing growth arrest or programmed cell death in infected hosts.

These findings substantially expanded the space of protein domains, molecular functions, and interactions that are employed by bacteria and archaea in antiviral defense. Some of these functions, including RNA editing, have not been previously implicated in defense mechanisms. The high success rate of defense system prediction based on the evolutionary conservation of their proximity to previously identified defense genes supported the defense island concept (4, 7, 10) and demonstrated its growing utility at the time of rapid expansion of sequence databases. Furthermore, the computational approach implemented in this work provided for a substantial expansion of the range of the identified putative defense systems. Many of these previously unknown defense systems contain enzymatic activities as well as predicted sensor components that potentially could be engineered for novel biotechnology applications.

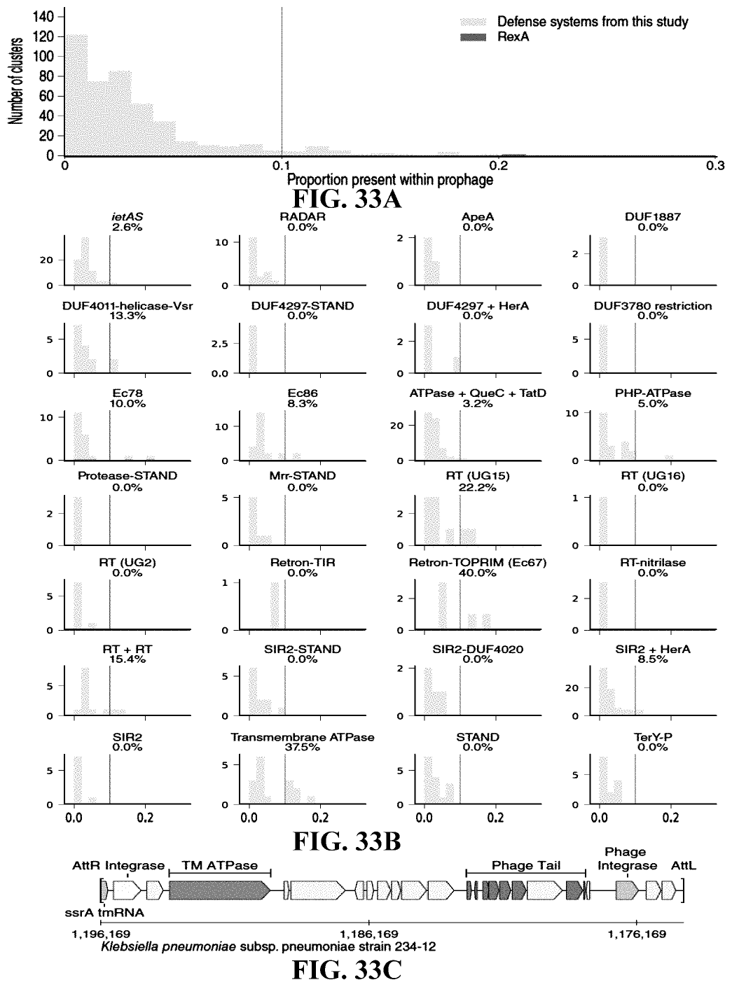

Despite similarities in domain architectures among some of the identified defense systems, their phage specificities differ significantly, emphasizing the importance of multiple defense mechanisms for the survival of prokaryotes in the arms race against viruses. These observations are compatible with the concept of distributed microbial immunity, according to which defense systems encoded in different genomes collectively protect microbial communities from the diverse viromes they confront (38). Additionally, several of the identified defense systems incorporate molecular functions from typically non-defense sources, highlighting the versatility of activities that are recruited for antiviral defense. These include the RADAR deaminase, nitrilases, and reverse transcriptases of different families, including retrons. The demonstration of defense functions for multiple RTs, which are generally associated with mobile genetic elements, is consistent with the ‘guns for hire’ paradigm whereby enzymes are shuttled between MGEs and defense systems during microbial evolution (8). Finally, most of these defense systems do not appear to be substantially enriched within prophages, suggesting that they are dedicated host defense genes, rather than virus superinfection exclusion modules (FIGS. 33A-33C and Methods).

The overall patchy pattern of phage specificity observed for the different defense systems was unexpected. In some cases, the same system exhibited widely varying levels of protection against similar phages; for instance, DRT type 3 offered full protection against phage T2 but no protection against phage T4, which is ˜98% identical to T2.

The range of domains contained within these systems indicates that they employ diverse biochemical activities. The identification of these defense systems, as well as others Applicants have predicted computationally, provides a foundation for mechanistic investigation.

The results described here have broad implications for understanding antiviral resistance and host-virus dynamics in natural populations of microbes, as well as for technological applications such as the development of anti-bacterial therapeutics, DNA and RNA editing, molecular detection, and targeted cell destruction.
1. C. A. Suttle, Viruses: unlocking the greatest biodiversity on Earth. Genome 56, 542-544 (2013).
2. A. G. Cobián Güemes et al., Viruses as Winners in the Game of Life. Annu Rev Virol 3, 197-214 (2016).
3. F. Hille et al., The Biology of CRISPR-Cas: Backward and Forward. Cell 172, 1239-1259 (2018).
4. S. Doron et al., Systematic discovery of antiphage defense systems in the microbial pangenome. Science 359, (2018).
5. J. E. Samson, A. H. Magadan, M. Sabri, S. Moineau, Revenge of the phages: defeating bacterial defences. Nat Rev Microbiol 11, 675-687 (2013).
6. J. Bondy-Denomy, A. Pawluk, K. L. Maxwell, A. R. Davidson, Bacteriophage genes that inactivate the CRISPR/Cas bacterial immune system. Nature 493, 429-432 (2013).
7. K. S. Makarova, Y. I. Wolf, E. V. Koonin, Comparative genomics of defense systems in archaea and bacteria. Nucleic Acids Res 41, 4360-4377 (2013).
8. E. V. Koonin, K. S. Makarova, Y. I. Wolf, M. Krupovic, Evolutionary entanglement of mobile genetic elements and host defence systems: guns for hire. Nat Rev Genet, (2019).
9. G. Faure et al., CRISPR-Cas in mobile genetic elements: counter-defence and beyond. Nat Rev Microbiol 17, 513-525 (2019).
10. K. S. Makarova, Y. I. Wolf, S. Snir, E. V. Koonin, Defense islands in bacterial and archaeal genomes and prediction of novel defense systems. J Bacteriol 193, 6039-6056 (2011).
11. S. A. Shmakov, K. S. Makarova, Y. I. Wolf, K. V. Severinov, E. V. Koonin, Systematic prediction of genes functionally linked to CRISPR-Cas systems by gene neighborhood analysis. Proc Natl Acad Sci USA 115, E5307-E5316 (2018).
12. S. A. Shmakov et al., Systematic prediction of functionally linked genes in bacterial and archaeal genomes. Nat Protoc 14, 3013-3031 (2019).
13. J. Gordeeva et al., BREX system of Escherichia coli distinguishes self from non-self by methylation of a specific DNA site. Nucleic Acids Res 47, 253-265 (2019).
14. T. Goldfarb et al., BREX is a novel phage resistance system widespread in microbial genomes. EMBO J 34, 169-183 (2015).
15. R. Odegrip, A. S. Nilsson, E. Haggard-Ljungquist, Identification of a gene encoding a functional reverse transcriptase within a highly variable locus in the P2-like coliphages. J Bacteriol 188, 1643-1647 (2006).
16. A. M. Burroughs, D. Zhang, D. E. Schïffer, L. M. Iyer, L. Aravind, Comparative genomic analyses reveal a vast, novel network of nucleotide-centric systems in biological conflicts, immunity and signaling. Nucleic Acids Res 43, 10633-10654 (2015).
17. K. S. Makarova, L. Gao, F. Zhang, E. V. Koonin, Unexpected connections between type VI-B CRISPR-Cas systems, bacterial natural competence, ubiquitin signaling network and DNA modification through a distinct family of membrane proteins. FEMS Microbiol Lett 366, (2019).
18. C. D. Rae, Y. Gordiyenko, V. Ramakrishnan, How a circularized tmRNA moves through the ribosome. Science 363, 740-744 (2019).
19. S. Zimmerly, L. Wu, An Unexplored Diversity of Reverse Transcriptases in Bacteria. Microbiol Spectr 3, MDNA3-0058-2014 (2015).
20. N. Toro, R. Nisa-Martinez, Comprehensive phylogenetic analysis of bacterial reverse transcriptases. PLoS One 9, e114083 (2014).
21. K. K. Kojima, M. Kanehisa, Systematic survey for novel types of prokaryotic retroelements based on gene neighborhood and protein architecture. Mol Biol Evol 25, 1395-1404 (2008).
22. D. M. Simon, S. Zimmerly, A diversity of uncharacterized reverse transcriptases in bacteria. Nucleic Acids Res 36, 7219-7229 (2008).
23. H. C. Pace, C. Brenner, The nitrilase superfamily: classification, structure and function. Genome Biol 2, REVIEWS0001 (2001).
24. A. J. Simon, A. D. Ellington, I. J. Finkelstein, Retrons and their applications in genome engineering. Nucleic Acids Res 47, 11007-11019 (2019).
25. F. Farzadfard, T. K. Lu, Synthetic biology. Genomically encoded analog memory with precise in vivo DNA writing in living cell populations. Science 346, 1256272 (2014).
26. B. C. Lampson et al., Reverse transcriptase in a clinical strain of Escherichia coli: production of branched RNA-linked msDNA. Science 243, 1033-1038 (1989).
27. D. Lim, W. K. Maas, Reverse transcriptase-dependent synthesis of a covalently linked, branched DNA-RNA compound in E. coli B. Cell 56, 891-904 (1989).
28. T. M. Lima, D. Lim, A novel retron that produces RNA-less msDNA in Escherichia coli using reverse transcriptase. Plasmid 38, 25-33 (1997).
29. L. Aravind, D. D. Leipe, E. V. Koonin, Toprim—a conserved catalytic domain in type IA and II topoisomerases, DnaG-type primases, OLD family nucleases and RecR proteins. Nucleic Acids Res 26, 4205-4213 (1998).
30. S. Horsefield et al., NAD. Science 365, 793-799 (2019).
31. V. Anantharaman, L. M. Iyer, L. Aravind, Ter-dependent stress response systems: novel pathways related to metal sensing, production of a nucleoside-like metabolite, and DNA-processing. Mol Biosyst 8, 3142-3165 (2012).
32. K. S. Makarova, Y. I. Wolf, J. van der Oost, E. V. Koonin, Prokaryotic homologs of Argonaute proteins are predicted to function as key components of a novel system of defense against mobile genetic elements. Biol Direct 4, 29 (2009).
33. V. Anantharaman, K. S. Makarova, A. M. Burroughs, E. V. Koonin, L. Aravind, Comprehensive analysis of the HEPN superfamily: identification of novel roles in intra-genomic conflicts, defense, pathogenesis and RNA processing. Biol Direct 8, 15 (2013).
34. L. Aravind, L. M. Iyer, D. D. Leipe, E. V. Koonin, A novel family of P-loop NTPases with an unusual phyletic distribution and transmembrane segments inserted within the NTPase domain. Genome Biol 5, R30 (2004).
35. D. D. Leipe, E. V. Koonin, L. Aravind, STAND, a class of P-loop NTPases including animal and plant regulators of programmed cell death: multiple, complex domain architectures, unusual phyletic patterns, and evolution by horizontal gene transfer. J Mol Biol 343, 1-28 (2004).
36. O. Danot, E. Marquenet, D. Vidal-Ingigliardi, E. Richet, Wheel of Life, Wheel of Death: A Mechanistic Insight into Signaling by STAND Proteins. Structure 17, 172-182 (2009).
37. E. V. Koonin, L. Aravind, Origin and evolution of eukaryotic apoptosis: the bacterial connection. Cell Death Differ 9, 394-404 (2002).
38. A. Bernheim, R. Sorek, The pan-immune system of bacteria: antiviral defence as a community resource. Nat Rev Microbiol 18, 113-119 (2020).
39. D. Hyatt et al., Prodigal: prokaryotic gene recognition and translation initiation site identification. BMC Bioinformatics 11, 119 (2010).
40. M. Punta et al., The Pfam protein families database. Nucleic Acids Res 40, D290-301 (2012).
41. A. Marchler-Bauer et al., CDD/SPARCLE: functional classification of proteins via subfamily domain architectures. Nucleic Acids Res 45, D200-D203 (2017).
42. M. Steinegger, J. Soding, MMseqs2 enables sensitive protein sequence searching for the analysis of massive data sets. Nat Biotechnol 35, 1026-1028 (2017).
43. M. Steinegger, J. Soding, Clustering huge protein sequence sets in linear time. Nat Commun 9, 2542 (2018).
44. R. J. Roberts, T. Vincze, J. Posfai, D. Macelis, REBASE—a database for DNA restriction and modification: enzymes, genes and genomes. Nucleic Acids Res 43, D298-299 (2015).
45. D. Cohen et al., Cyclic GMP-AMP signalling protects bacteria against viral infection. Nature, (2019).
46. G. Ofir et al., DISARM is a widespread bacterial defence system with broad anti-phage activities. Nat Microbiol 3, 90-98 (2018).
47. K. Katoh, K. Misawa, K. Kuma, T. Miyata, MAFFT: a novel method for rapid multiple sequence alignment based on fast Fourier transform. Nucleic Acids Res 30, 3059-3066 (2002).
48. L. Zimmermann et al., A Completely Reimplemented MPI Bioinformatics Toolkit with a New HHpred Server at its Core. J Mol Biol 430, 2237-2243 (2018).
49. J. C. Petricciani, F. C. Chu, J. B. Johnson, H. M. Meyer, Bacteriophages in live virus vaccines. Proc Soc Exp Biol Med 144, 789-792 (1973).
50. J. B. Milstien, J. R. Walker, J. C. Petricciani, Bacteriophages in live virus vaccines: lack of evidence for effects on the genome of rhesus monkeys. Science 197, 469-470 (1977).
51. B. Xu, X. Ma, H. Xiong, Y. Li, Complete genome sequence of 285P, a novel T7-like polyvalent E. coli bacteriophage. Virus Genes 48, 528-533 (2014).
52. S. Picelli et al., Tn5 transposase and tagmentation procedures for massively scaled sequencing projects. Genome Res 24, 2033-2040 (2014).
53. E. S. Miller et al., Bacteriophage T4 genome. Microbiol Mol Biol Rev 67, 86-156 (2003).
54. D. H. Turner, D. H. Mathews, NNDB: the nearest neighbor parameter database for predicting stability of nucleic acid secondary structure. Nucleic Acids Res 38, D280-282 (2010).
55. Y. Zhou, Y. Liang, K. H. Lynch, J. J. Dennis, D. S. Wishart, PHAST: a fast phage search tool. Nucleic Acids Res 39, W347-352 (2011).
56. D. Arndt et al., PHASTER: a better, faster version of the PHAST phage search tool. Nucleic Acids Res 44, W16-21 (2016).
57. J. Strecker et al., RNA-guided DNA insertion with CRISPR-associated transposases. Science 365, 48-53 (2019).
58. S. E. Klompe, P. L. H. Vo, T. S. Halpin-Healy, S. H. Sternberg, Transposon-encoded CRISPR-Cas systems direct RNA-guided DNA integration. Nature 571, 219-225 (2019).
59. E. V. Koonin, K. S. Makarova, Y. I. Wolf, Evolutionary Genomics of Defense Systems in Archaea and Bacteria. Annu Rev Microbiol 71, 233-261 (2017).
60. S. Yamamoto, K. Kiyokawa, K. Tanaka, K. Moriguchi, K. Suzuki, Novel toxin-antitoxin system composed of serine protease and AAA-ATPase homologues determines the high level of stability and incompatibility of the tumor-inducing plasmid pTiC58. J Bacteriol 191, 4656-4666 (2009).

Materials and Methods

Detection of known defense systems. All bacterial and archaeal genomes (n=174,080) were downloaded from Genbank (NCBI) in November 2018. For genomes where gene annotations were incomplete or missing, genes were predicted using Prodigal (39). Known defense-related protein domains were annotated using RPSBLAST version 2.8.1 and the set of position-specific scoring matrices curated from the NCBI Conserved Domain Database (CDD) (4, 10, 40, 41). To reduce the false positive rate, a multi-gene system containing a ubiquitous protein domain was required to include two or more of its component genes in close proximity. For example, the type I restriction-modification endonuclease hsdR was called as a defense gene only if the corresponding methylase (hsdM) or specificity protein (hsdS) was also encoded in the vicinity. Genes were predicted for known defense systems including HsdRMS, McrBC, BREX, Druantia, Zorya, Wadjet, Thoeris, Hachiman, Lamassu, Gabjia, Septu, Shedu, Kiwa, pAgo, and other RM systems. Toxin-antitoxin systems were excluded from the set of known systems due to their overall low enrichment within defense islands (FIGS. 21A-21D).

Candidate novel defense genes. All translated protein-coding sequences within either 10 kb or 10 ORFs of known defense systems (whichever was greater), including the components of the known defense systems themselves, were compiled into a preliminary list (8.7×10⁶ genes), which was expected to consist of both defense and non-defense genes. Highly similar sequences (at least 98% sequence identity and coverage) were discarded using the linclust option in MMseqs2 (42, 43) with parameters—min-seq-id 0.98-c 0.98, resulting in a reduced list of 2.5×10⁶ sequences. These sequences were then further clustered using the cascaded clustering option in MMSeqs2, yielding a final list of 6.0×10⁵ representatives (“seeds”).

Scoring candidate genes for defense enrichment. For each of the 6.0×10⁵ seeds, a “defense enrichment score” was computed as (number of homologs in proximity to one or more known defense systems)/(total number of homologs). A gene was considered to be located in proximity to a known defense system if it occurred no more than 5 kb or 5 ORFs away from the locus encoding that system. CRISPR-Cas systems were omitted from the defense score calculation due to their low defense island association (10). Candidate sequences with a defense enrichment score of 0.1 or higher were retained for subsequent analysis, with the exception of predicted mobilome components (such as transposons), which were discarded. This cut-off was chosen because more than 90% of the known defense genes scored higher than this value, whereas most mobilome, toxin-antitoxin, and other non-defense genes scored lower (FIGS. 16B, 21A-21D). To identify homologs of the candidate proteins, all 6.2×10⁸ proteins in Genbank were tabulated, and highly similar proteins (at least 98% sequence identity and coverage) were removed, resulting in a reduced list of 1.3×10⁸ proteins. Each seed sequence was then searched against this non-redundant protein sequence database using MMseqs2. To qualify as evidence of homology, the resulting alignments were required to have a minimum coverage of 70% and a maximum E value of 10⁻⁵ (parameters—coy-mode 0-c 0.7-e 0.00001). The set of identified homologs was further clustered at 90% sequence identity to perform stringent redundancy reduction. In order to accurately compute defense association frequencies, seeds with fewer than 50 homologs after redundancy reduction were discarded.

Filtering defense-enriched genes based on context diversity. To select for genes that are likely to encode components of independent defense modules, defense-enriched seeds were further required to have sufficient context diversity. For each seed, the number of homologs within 5 kb or 5 ORFs of different defense system categories was counted, and the seed was retained if the entropy of this list, defined as Σ−pᵢln pᵢ, where pᵢ is the normalized frequency of category i, was at least 0.9. This value corresponds to halfway between 2 and 3 non-zero entries in the case of a uniformly distributed frequency vector. Seeds were further filtered based on the proportion of homologs next to predicted toxin-antitoxin/Abi, mobilome, and CRISPR-Cas genes (FIGS. 21A-21D).

Refining the classification of putative defense genes. A total of 12,027 seeds passing filter was identified, consisting of both known and putative defense genes. To determine whether each gene was putative or known, the original classification was refined as follows. A list was compiled of the amino acid sequences of reported homologs of known systems, including 288,776 restriction-modification proteins from REBASE (44); 517 proteins for BREX (14); and 27,775 proteins for other recently-identified systems (4, 45, 46). This list was supplemented with additional curated homologs and, following redundancy reduction, searched against the putative defense seeds using MMseqs2. Seeds that matched one or more of these known defense genes (at least 70-80% coverage with a maximum E value of 10⁻⁵) were labeled as known. A subset of labels were adjusted by an additional round of manual curation, resulting in a classification of 4,555 known and 7,472 putative defense genes.

Domain analysis of predicted defense genes. The 7,472 putative defense seeds were further analyzed with additional, more sensitive methods to assess their domain content. For each seed gene, a multiple sequence alignment (MSA) of its homologs was created using MAFFT (47). If the number of homologs was 1,000 or fewer, all homologs were included in the alignment; otherwise, 1,000 homologs were randomly selected for inclusion. MSAs were searched against the Pfam 32.0 database using HHpred (48), and domain predictions with at least 80% probability were retained. Of these 7,472 genes, 3,029 (41%) contained at least one pfam domain that has been reported to be defense-associated (4, 10, 45). Although some of these 3,029 proteins could be distant homologs of known defense proteins, many were included in this category because they contained ubiquitous pfam domains that are also employed by some known defense systems (in particular, AAA-family ATPases, helix-turn-helix (HTH) motifs, and (P)D−(D/E)×K-family nucleases); these are predicted to be uncharacterized defense genes. The remaining 59% either had no domain hits or contained only domains that were not in the set of defense-associated pfams.

From genes to defense systems. For each selected candidate defense protein, the gene neighborhoods of 30 homologs in proximity to known defense genes were randomly chosen and examined to identify conserved (predicted) operons that contained the seed and could be expected to constitute a minimal, intact defense system. Protein domains were predicted using HHpred, and the resulting prediction was used to infer the potential involvement of the respective proteins in the activity of the respective predicted defense system.

Estimation of defense system abundance. To estimate the abundance of each validated defense system in microbial genomes, Applicants downloaded n=205,214 genomes available in Genbank as of August 2019. For each defense system, initial protein sequence seeds encoded by the corresponding signature genes were taken from experimentally validated loci. Initial seeds were aligned and converted into HMM profiles. Applicants then used a constrained 2 iteration HMM profile search to generate highly specific HMM profiles and retrieve related systems as follows. Each ORF of size 150aa or greater, with one or more hits, was searched against all MINI profiles using HMMER3.1 and assigned to the profile that had the highest scoring match. For each system, ORFs with profile hits with less than 500 bp of intergenic distance on the same strand were grouped into candidate loci. For multi-protein systems, a putative locus was considered a hit if every signature gene profile for the system had a match in the locus with a bit score of at least 25. For single gene systems, a locus was considered a hit if the protein had a match to the system's single signature gene profile with a bit score of at least 50 and an alignment coverage of at least 70%. Signature proteins from the identified systems were separately clustered at 50% identity using MMseqs2 and subsequently aligned using MAFFT. The alignments were used to create a new set of signature gene profiles as input to the next iteration. For BREX and Type I RM, Applicants used preexisting pfam profiles for the signature genes in place of iterative MINI profile searching. The final abundance was calculated as the number of hits for the given system divided by the number of genomes (n).

Bacteria and phage strains. Phages T2, T3, T4, T5, T7, P1, λ, φV-1, M13, φX174, MS2, and Qβ, as well as host E. coli strains K-12 (ATCC25404) and C (ATCC13706), were obtained from the American Type Culture Collection (ATCC). The genome of phage φV-1, originally isolated from a measles vaccine (49, 50), was sequenced and found to be 92% similar to enterobacteria phage 285P, a T7-like phage (51).

Cloning. To facilitate experimental validation using coliphages, the source organism of each candidate defense system was chosen to be as phylogenetically similar as possible to E. coli, in particular, from other strains of E. coli whenever possible. Candidate defense systems were cloned into the low-copy plasmid pACYC184. When possible, genomic DNA from source organisms was obtained from ATCC, NCTC, or DSMZ, and the genes of interest were amplified with Q5 (New England Biolabs) or Phusion Flash (Thermo Scientific) polymerase, using primers with 5′ ends homologous to the ends of the plasmid backbone. Plasmids were assembled using the NEBuilder HiFi DNA Assembly mix (New England Biolabs). When the source organism was not readily available from public culture collections, genes were chemically synthesized (GenScript). When possible, the native promoter was retained. For source organisms outside of Enterobacteriaceae, or when the candidate system was operonized with other upstream genes, the system was placed under a bla or lac promoter.

Sequence verification of plasmids. The full sequences of all plasmids were verified by high-throughput sequencing. To prepare sequencing libraries, 25-50 ng of each plasmid was mixed with purified Tn5 transposome loaded with Illumina adapters and incubated at 55° C. for 10 min in the presence of 5 mM MgCl2 and 10 mM TAPS buffer (52). The quantity of Tn5 was titrated to generate an average fragment size of ˜100-400 bp. Tagmentation reactions were subsequently treated with 0.5 volumes of 0.1% sodium dodecyl sulfate for 5 min at room temperature and amplified with KAPA HiFi HotStart polymerase using primers containing 8 nt i7 and i5 index barcodes. Barcoded amplicons were sequenced on a MiSeq (Illumina) with at least 150 cycles for the forward read. Reads were aligned to the reference plasmid sequence by the Geneious read mapper, and error-free plasmids were retained for subsequent experiments.

Competent cell production. E. coli strains K-12 and C were cultured in ZymoBroth with 25 μg/mL chloramphenicol and made competent using Mix & Go buffers (Zymo) according to the manufacturer's recommended protocol.

Phage plaque assays. E. coli host strains were grown to saturation at 37° C. in Luria Broth (LB). To 10 mL top agar (10 g/L tryptone, 5 g/L yeast extract, 10 g/L NaCl, 7 g/L agar) was added chloramphenicol (final concentration 25 μg/mL) and 526 μL E. coli culture, and the mixture was poured on 10 cm LB-agar plates containing 25 μg/mL chloramphenicol. For phages T2, T4, T5, P1, λ, M13, MS2, and Qβ, dilutions of phage in phosphate buffered saline were spotted on the plates, and plaque counts were recorded after overnight incubation at 37° C. If individual plaques were too small to be counted, the most concentrated dilution at which no plaque formation was visible was recorded as having a single plaque. For phages T3, T7, φV-1, and φX174, a total of 3 of phage containing 5×10⁶ virions was spotted, and the area of the zone of lysis was measured after incubation at 37° C. for 68 hr. A total of 2-4 technical replicates was collected for each infection condition. Initial screening of defense system candidates was performed in E. coli K-12 (ATCC25404), excluding phage φX174 due to its inability to infect E. coli K-12; systems with observed defense activity were further tested as described above.

Phage cultivation. Phages T2, T3, T4, T7, φV-1, M13, φX174, MS2, and Qβ were propagated in liquid culture. The host E. coli strain for each phage was grown to an OD600 of 0.2 -0.4 at 37° C. in LB and infected with a slab of top agar containing phage plaque from a previous lysis. Cultures were grown overnight at 37° C. with 250 rpm agitation. Phages T5, P1, and λ were propagated by the double agar overlay method; after overnight incubation at 37° C., plaques were scraped in LB. For both liquid culture and double agar overlay, phage samples were centrifuged to pellet cellular debris, and the supernatant was filtered through with a 0.22 μm sterile filter.

Phage genome sequencing. DNA from phage φV-1 was isolated using QuickExtract DNA extraction solution (Epicentre) following the manufacturer's recommended protocol. After tagmentation and PCR amplification steps described earlier for plasmid sequence verification, the library was sequenced on a MiSeq with 200 cycles for the forward read and 110 cycles for the reverse read. Trimmed reads were assembled into contigs with SPAdes 3.13.0 using the—careful option, and contigs were subsequently scaffolded into a full genome using the genome sequence of enterobacteria phage 285P (51) as a reference.

Whole transcriptome sequencing. E. coli ATCC25404, containing either an empty vector or the candidate defense system, was grown to log phase in LB and diluted to an OD600 of 0.2. The culture was then split into two tubes, one of which was infected with phage T2 at an estimated MOI of 2. Both subcultures were incubated at 37° C. for 1 hr with 250 rpm agitation. RNA was extracted using TRIzol Reagent (Thermo Fisher Scientific) and treated with DNAse I, followed by a RiboMinus ribosomal RNA depletion kit (Thermo). Sequencing libraries were prepared using NEB Ultra II directional RNAseq library prep kit (New England Biolabs) and paired-end sequenced (2×75 cycles) with a NextSeq (Illumina). Adapter sequences were trimmed from sequencing reads using CutAdapt (with parameters—trim-n-q 20-m 20-a AGATCGGAAGAGC-A AGATCGGAAGAGC (SEQ ID NO: 472)), and trimmed reads were aligned to the E. coli MG1655 reference genome using the Geneious read mapper.

Phage fragmentation. Phage fragments were amplified from the genome of phage T2 by PCR, cloned into an ampicillin-resistant plasmid after an IPTG-inducible T7 promoter, and sequenced verified as previously described. Each fragment was then transformed into NovaBlue(DE3) E. coli expressing the Citrobacter rodentium RADAR system. Independent colonies for each fragments were grown to saturation at 37° C. in LB with 25 μg/mL chloramphenicol and 100 μg/mL ampicillin. Cultures were then diluted 1 to 5 in the same media, and IPTG was added to a final concentration of 0.5 mM. After 4 h growth at 37° C., cells were pelleted by centrifugation, and total RNA was extracted by a Direct-zol RNA purification kit (Zymo). The E. coli tmRNA was subsequently amplified by RT-PCR (QuantBio) and sequenced with a MiSeq (Illumina).

E. coli growth kinetics. Cells were grown to log phase in LB and diluted to an OD600 of 0.2. Cultures were infected with phage T2 at varying MOI at grown at 37° C., and the OD600 was measured every 2 min for a total duration of 4 hr on a Synergy Neo2 plate reader (BioTek).

Classification of phage genes. Phage T2 genes were classified as putative early, middle, or late genes based on the closest promoter on the same strand, as annotated based on the genome of phage T4 (53). Genes that could not be unambiguously classified were labeled as unknown.

RNA secondary structure prediction. Minimum free energy RNA secondary structures were predicted using the Turner (2004) energy parameters at 37° C. (54).

Prophage analysis. Prophage and phage DNA sequences were downloaded from PHASTER (55, 56). All clusters (seed gene plus identified homologs) with hits matching the experimentally validated systems, as well as one cluster matching the rexA gene of phage lambda as a positive control, were searched against the PHASTER database with tblastn for near identical matches (≥95% identity). For each cluster, phage association frequency was calculated as the number of proteins in the cluster with unique matches to the PHASTER database divided by the total number of unique proteins in the cluster (number of proteins after clustering at 90% sequence identity). The cutoff for frequent phage association of a system was defined as half of the frequency for rexA. Applicants note that PHASTER does not predict all instances of prophages and prophage remnants, and Applicants have also considered an alternative approach of identifying prophage association based on proximity to integrases, which may allow a greater number of prophages to be identified. However, a challenge with the latter approach is that defense islands often appear to derive from mobile genetic elements other than prophages and contain many integrases that originate from non-phage sources (e.g., CRISPR-associated transposases (57, 58)), leading to a high rate of false positives. The use of PHASTER provided the advantage of substantially reducing the false positives that would otherwise be expected for an approach based on integrase association.

Computational analysis of the RT (UG1) nitrilase domain. Homologs of the RT (UG1) defense gene were identified with a PSIBLAST search seeded on the experimentally validated sequence (WP_115196278.1), and highly similar homologs (≥90% identity) were removed. An MSA of the nitrilase domain was then created using MAFFT, and a custom position-specific scoring matrix (PSSM) was derived from this alignment. Bacterial and archaeal proteins in Genbank (redundancy-reduced at 98% sequence identity and coverage) were then searched against this profile with RPSBLAST, and the E-values of proteins with a match covering a minimum of 20% of the length of the profile were recorded. Known nitrilase enzymes were identified using a separate RPSBLAST search against the same set of Genbank proteins using 36 PSSMs from the CDD database (E-value≤10⁻⁶; minimum 40% profile coverage): cd07197, cd07564, cd07565, cd07566, cd07567, cd07568, cd07569, cd07570, cd07571, cd07572, cd07573, cd07574, cd07575, cd07576, cd07577, cd07578, cd07579, cd07580, cd07581, cd07582, cd07583, cd07584, cd07585, cd07586, cd07587, COG0388, pfam00795, PLN02504, PLN02747, PLN02798, PRK10438, PRK13286, PRK13825, TIGR00546, TIGR03381, and TIGR04048.

Establishing an abi response. Abortive infection (abi) systems, which are based on altruistic cell suicide or dormancy (59), typically induce non-specific or deleterious biochemical activity targeting the host cell that also interferes with the phage reproduction cycle. Abi responses can be characterized through traditional assays such as efficiency of the center of infection (ECOI), adsorption, host survival, and one-step growth curve measurements. However, because the events of phage DNA injection and expression of toxic early genes are likely to be deleterious to an infected cell even if the production of progeny phages is ultimately suppressed, these assays may not be informative in terms of distinguishing between abi vs. non-abi mechanisms. An alternative approach to establishing the existence of an abi response is to identify the biochemical activity of the defense system, which Applicants have focused on for the RADAR system.

Gene knockouts vs. heterologous reconstitution. To further assess the feasibility of performing knockout experiments in the source bacterial strains for each defense system, Applicants performed analyses which suggested that different defense systems with overlapping phage specificities often co-occur. For instance, E. coli strain DSM5212 contains both BREX type I and Druantia type I (FIG. 2D), both of which were included as positive controls; if BREX were to be knocked out in this strain, the presence of Druantia would likely ensure that its phage resistance profile across the 12 phages in Applicants' assay would remain unchanged. Similarly, the SIR2+HerA system from E. coli strain NCTC11129 primarily confers resistance to phage lambda (FIG. 2D); the source strain NCTC11129 additionally contains BREX type I, which also confers resistance against phage lambda. Collectively, these observations suggested that the knockout of a single defense system may not be sufficient to make its corresponding source strain phage-sensitive, motivating the use of heterologous reconstitution as the primary assay for defense activity.

TABLE 9
List of validated defense systems and their domain architectures.

| # | WT | Mutants | Type | Name | Domain Architecture* |
|---|---|---|---|---|---|
| 1 | FIG. 17D | FIG. 19B | Retron | Retron-TIR | RT_etron-TIR |
| 2 | FIG. 17D | FIG. 19B | Retron | Ec67 | RT_retron-TOPRIM |
| 3 | FIG. 17D | FIG. 19B | Retron | Ec86 | Nuc_deoxy + RT_retron |
| 4 | FIG. 17D | FIG. 29C | Retron | Ec78 | RT_retron + ATPase_AAA + HNH |
| 5 | FIG. 17D | FIG. 19B | RT | DRT type 1 | RT_UG1-nitrilase |
| 6 | FIG. 17D | FIG. 29A | RT | DRT type 2 | RT_UG2 |
| 7 | FIG. 17D | FIG. 19B | RT | DRT type 3 | RT_UG3 + RT_UG8 |
| 8 | FIG. 17D | FIG. 29B | RT | DRT type 4 | RT_UG15 |
| 9 | FIG. 17D | FIG. 19B | RT | DRT type 5 | RT_UG16 |
| 10.A | FIG. 17D | FIG. 18B | RNA | RADAR | ATPase_AAA + ADA |
| 10.B | FIG. 18B | FIG. 18B | RNA | RADAR | ATPase_AAA + ADA |
| 11 | FIG. 17D | FIG. 20 | RNA | apeA | RNase_ApeA |
| 12 | FIG. 17D | FIG. 20 | STAND | AVAST type 1 | MBL + Protease_S1-ATPase_STAND |
| 13 | FIG. 17D | FIG. 20 | STAND | AVAST type 2 | ATPase_STAND |
| 14 | FIG. 17D | FIG. 20 | STAND | AVAST type 3 | Nuclease_DUF4297-ATPase_STAND |
| 15 | FIG. 17D | FIG. 20 | STAND | AVAST type 4 | Nuclease_Mrr-ATPase_STAND |
| 16 | FIG. 17D | FIG. 20 | STAND | AVAST type 5 | SIR2-ATPase_STAND |
| 17 | FIG. 17D | FIG. 20 | Other | dsr1 | SIR2-DUF4020 |
| 18 | FIG. 17D | FIG. 20 | Other | dsr2 | SIR2 |
| 19 | FIG. 17D | FIG. 20 | Other | SIR2 + HerA | SIR2 + Helicase_HerA |
| 20 | FIG. 17D | FIG. 20 | Other | DUF4297 + | Nuclease_DUF4297 + Helicase_HerA |
| | | | | HerA | |
| 21 | FIG. 17D | FIG. 20 | Other | tmn | ATPase_AAA_TM |
| 22 | FIG. 17D | FIG. 20 | Other | qatABCD | ATPase_AAA + QueC + DNase_TatD |
| 23 | FIG. 17D | FIG. 20 | Other | hhe | HEPN_DUF4011-Helicase_SF1_Dna2- |
| | | | | | Nuclease_Vsr-DUF3320 |
| 24 | FIG. 17D | — | Other | mzaABCDE | Ankyrin-sigma + ATPase_MutL + |
| | | | | | ATPase_AAA-Z1 + |
| | | | | | Nuclease_DUF4420 + AIPR |
| 25 | FIG. 17D | FIG. 20 | Other | TerY-P | vWA + phosphatase_PP2C + STK-OB |
| 26 | FIG. 17D | FIG. 20 | Other | upx | Nuclease_DUF1887 |
| 27 | FIG. 17D | FIG. 20 | Other | ppl | Phosphoesterase_PHP-ATPase_SMC |
| 28 | FIG. 17D | FIG. 20 | Other | ietAS** | ATPase_AAA + Protease_S8 |
| 29 | FIG. 17D | FIG. 20 | Other | Restriction- | ATPase_DUF499 + DUF3780 + |
| | | | | like system | Methylase_DUF1156 + Nuclease_PLD- |
| | | | | | Helicase_HepA |

*Dashes (-) indicated domain fusions and (+) represents separate proteins.
**ietAS is also a previously-described plasmid stabilization toxin-antitoxin system (60).

TABLE 10
Source organism strains of validated defense systems and controls.

| # | Source Organism | Strain | Promoter | Codon | Genes | bp |
|---|---|---|---|---|---|---|
| BREX | Escherichia coli | DSM5212 | Native | Native | 6 | 13703 |
| type I | | | | | | |
| Druantia | Escherichia coli | DSM5212 | Native | Native | 5 | 11823 |
| type I | | | | | | |
| RT-Abi-P2 | Escherichia coli | ECOR30 | Native | Native | 1 | 1921 |
| 1 | Shigella dysenteriae | NCTC2966 | Native | Native | 1 | 2064 |
| 2 | Escherichia coli | NCTC8623 | Native | Native | 1 | 2038 |
| 3 | Escherichia coli | BL21 | Native | Native | 2 | 2188 |
| 4 | Escherichia coli | ECONIH5 | Native | Native | 3 | 3551 |
| 5 | Klebsiella pneumoniae | NCTC9143 | Native | Native | 2 | 4451 |
| 6 | Salmonella enterica | NCTC8273 | Native | Native | 1 | 1780 |
| 7 | Escherichia coli | ECOR12 | Native | Native | 2 | 4995 |
| 8 | Escherichia coli | 21-C8-A | Native | Human | 1 | 1838 |
| 9 | Escherichia coli | KTE25 | Native | Native | 1 | 1608 |
| 10.A | Citrobacter rodentium | DBS100 | Native | Native | 2 | 5526 |
| 10.B | Pluralibacter gergoviae | ATCC33028 | Native | Native | 3 | 6689 |
| 11 | Escherichia coli | NCTC8008 | Native | Native | 1 | 1981 |
| 12 | Erwinia piriflorinigrans | CFBP5888 | bla | Native | 3 | 7246 |
| 13 | Escherichia coli | NCTC9087 | Native | Native | 1 | 5109 |
| 14 | Salmonella enterica | NCTC13175 | Native | Native | 2 | 7175 |
| 15 | Escherichia coli | NCTC11132 | Native | Native | 1 | 4964 |
| 16 | Escherichia coli | NCTC13384 | Native | Native | 1 | 3411 |
| 17 | Escherichia coli | NCTC9112 | Native | Native | 1 | 4212 |
| 18 | Cronobacter sakazakii | NCTC8155 | Native | Native | 1 | 4329 |
| 19 | Escherichia coli | NCTC11129 | Native | Native | 2 | 3308 |
| 20 | Escherichia coli | NCTC11131 | Native | Native | 2 | 3419 |
| 21 | Escherichia coli | ECOR25 | Native | Native | 1 | 4415 |
| 22 | Escherichia coli | NCTC9009 | Native | Native | 4 | 5408 |
| 23 | Escherichia coli | ATCC43886 | Native | Native | 1 | 5958 |
| 24 | Salmonella enterica | NCTC5773 | Native | Native | 5 | 9416 |
| 25 | Citrobacter gillenii | NCTC9094 | Native | Native | 3 | 3605 |
| 26 | Salmonella enterica | NCTC6026 | Native | Native | 1 | 4100 |
| 27 | Escherichia coli | NCTC8620 | Native | Native | 1 | 3066 |
| 28 | Escherichia coli | ECOR52 | Native | Native | 2 | 3676 |
| 29 | Escherichia coli | ECOR58 | Native | Native | 4 | 9809 |

TABLE 11
PCR primers used to amplify validated defense systems and controls.

| # | dfd | Sequence |
|---|---|---|
| BREX | Fwd | gctaacttacattaattgcgttgcgcaACAGCACCACGTTCATCTTCC |
| type I | | (SEQ ID NO: 14) |
| | Rev | ccaaggggttatgctagttattgcgGTTCATTAAAATAGTTACTACGTTAATTCACACCC |
| | | (SEQ ID NO: 215) |
| Druantia | Fwd | gctaacttacattaattgcgttgcgcaGGTGAACGTTTGGTTGATAGGG |
| type I | | (SEQ ID NO: 216) |
| | Rev | ccaaggggttatgctagttattgcgCTCAATGGGCATAATTTTACATTGTGC |
| | | (SEQ ID NO: 217) |
| RT-Abi- | Fwd | gctaacttacattaattgcgttgcgcaACATCCCGTCATCATGCCATC |
| P2 | | (SEQ ID NO: 218) |
| | Rev | ccaaggggttatgctagttattgcgCTCCTCGGAATAGAATGTTATGTTCG |
| | | (SEQ ID NO: 219) |
| 1 | Locus synthesized | |
| 2 | Fwd | gctaacttacattaattgcgttgcgcaCGCGCTATCACGTAAAATAGGC |
| | | (SEQ ID NO: 220) |
| | Rev | ccaaggggttatgctagttattgcgCGAAAAATCAGCCTTAGCGTTCATAAC |
| | | (SEQ ID NO: 221) |
| 3 | Fwd | gctaacttacattaattgcgttgcgcaGCTCATGTTATGCATGTGCATG |
| | | (SEQ ID NO: 222) |
| | Rev | ccaaggggttatgctagttattgcgATTAGGTCTTCGCTTTATTTAAAGGGTTC |
| | | (SEQ ID NO: 223) |
| 4 | Locus synthesized | |
| 5 | Fwd | gagctaacttacattaattgcgttgcgcaGTCCTTAAACACGACAAAACCTGTG |
| | | (SEQ ID NO: 224) |
| | Rev | cccaaggggttatgctagttattgcgCGCAATGTAACACCCACCC |
| | | (SEQ ID NO: 225) |
| 6 | Locus synthesized | |
| 7 | Fwd | gctaacttacattaattgcgttgcgcaTCTCAACTTCCCCAAATGTCCG |
| | | (SEQ ID NO: 226) |
| | Rev | cccaaggggttatgctagttattgcgTTAGCAAAATACGCCCACGAAGTC |
| | | (SEQ ID NO: 227) |
| 8 | Locus synthesized | |
| 9 | Locus synthesized | |
| 10.A | Fwd | gctaacttacattaattgcgttgcgcaGAGGATTTATGCACAAAATCCTGATGC |
| | | (SEQ ID NO: 228) |
| | Rev | ccaaggggttatgctagttattgcgGATTTAATCTGTTGTTCCGAACGG |
| | | (SEQ ID NO: 229) |
| 10.B | Fwd | gctaacttacattaattgcgttgcgcaTGTGGTTAGTTATCACAGCACTAACC |
| | | (SEQ ID NO: 230) |
| | Rev | ccaaggggttatgctagttattgcgGTGTATAAGAATCCGAGACCGAAC |
| | | (SEQ ID NO: 231) |
| 11 | Locus synthesized | |
| 12 | Fwd | ataaatgctcaataatattgaaaaaggaagagtATGGTAGCGATAAAAATGTATCCGGC |
| | | (SEQ ID NO: 232) |
| | Rev | cccaaggggttatgctagttattgcgTCAATCCGTAGCCTCTTCATTCTCG |
| | | (SEQ ID NO: 233) |
| 13 | Fwd | gctaacttacattaattgcgttgcgcaGGGATTTCCACCACCTCCC |
| | | (SEQ ID NO: 234) |
| | Rev | ccaaggggttatgctagttattgcgTGCATAGCAATGAAGATAAACGTG |
| | | (SEQ ID NO: 235) |
| 14 | Fwd | gctaacttacattaattgcgttgcgcaACAATTTTTTGCCATAAGACGCTTTC |
| | | (SEQ ID NO: 236) |
| | Rev | ccaaggggttatgctagttattgcgCATTAGGACTAGTAGAAAAGTCTTGGG |
| | | (SEQ ID NO: 237) |
| 15 | Fwd | gctaacttacattaattgcgttgcgcaGCGCAGCTGACAAAGATTGAC |
| | | (SEQ ID NO: 238) |
| | Rev | ccaaggggttatgctagttattgcgCGATAATAAAAAGGCTCCAATCCCTG |
| | | (SEQ ID NO: 239) |
| 16 | Fwd | gctaacttacattaattgcgttgcgcaACTAGCTAAGCAATAAGGGCG |
| | | (SEQ ID NO: 240) |
| | Rev | ccaaggggttatgctagttattgcgCAATCTCCGAGGTGGCCC |
| | | (SEQ ID NO: 241) |
| 17 | Fwd | gctaacttacattaattgcgttgcgcaTATTTTGCGTAGCTAGAACGCAATC |
| | | (SEQ ID NO: 242) |
| | Rev | ccaaggggttatgctagttattgcgTGGGTATTAGCTCATATCAGAACTAATACCC |
| | | (SEQ ID NO: 243) |
| 18 | Fwd | gctaacttacattaattgcgttgcgcaGTAAGACAAGGGTTGAGCAGGC |
| | | (SEQ ID NO: 244) |
| | Rev | ccaaggggttatgctagttattgcgCAATGGTGGGCTGATTAATTAGATGAG |
| | | (SEQ ID NO: 245) |
| 19 | Fwd | gctaacttacattaattgcgttgcgcaTAGCTATTGTGACTATGCTAACCATATG |
| | | (SEQ ID NO: 246) |
| | Rev | ccaaggggttatgctagttattgcgTTCAGTCTAAATACATACCTGTCGGG |
| | | (SEQ ID NO: 247) |
| 20 | Fwd | gctaacttacattaattgcgttgcgcaGTGCGCCTTATGTGATTACAACG |
| | | (SEQ ID NO: 248) |
| | Rev | ccaaggggttatgctagttattgcgCTCTCAGCCTAATGATTCCAGAATAG |
| | | (SEQ ID NO: 249) |
| 21 | Fwd | gctaacttacattaattgcgttgcgcaACCGTGCTGGCATGTTTTTAC |
| | | (SEQ ID NO: 250) |
| | Rev | ccaaggggttatgctagttattgcgAGGAAGATCCGTGACCAGGAG |
| | | (SEQ ID NO: 251) |
| 22 | Fwd | gctaacttacattaattgcgttgcgcaGAAATTATTTGGAATGGATGATGGCG |
| | | (SEQ ID NO: 252) |
| | Rev | ccaaggggttatgctagttattgcgACTTCTACCTCCCTTTAGAAAAGTTAATG |
| | | (SEQ ID NO: 253) |
| 23 | Fwd | gctaacttacattaattgcgttgcgcaCGGATTGAATCTGTTTATGAAATTTGGCTG |
| | | (SEQ ID NO: 254) |
| | Rev | ccaaggggttatgctagttattgcgCCGACAGTTGTCACTGTTCTTATTACC |
| | | (SEQ ID NO: 255) |
| 24 | Fwd | tgagctaacttacattaattgcgttgcgcaATGATGAAGATCACCTAAAATGATAGGTTG |
| | | (SEQ ID NO: 256) |
| | Rev | cccaaggggttatgctagttattgcgCAGCTGTTAATTGTATATTGATGCGATGC |
| | | (SEQ ID NO: 257) |
| 25 | Fwd | gctaacttacattaattgcgttgcgcaCGTGATGAATGAAGCGGCTAAATAC |
| | | (SEQ ID NO: 258) |
| | Rev | ccaaggggttatgctagttattgcgGTAAATCCTCGGGAAAACACAGG |
| | | (SEQ ID NO: 259) |
| 26 | Fwd | gctaacttacattaattgcgttgcgcaGGGCTGTTTGGTTGAATTAAAAATACG |
| | | (SEQ ID NO: 260) |
| | Rev | ccaaggggttatgctagttattgcgCCTTGATTTAAAACTATCAGTAGTAGGAACG |
| | | (SEQ ID NO: 261) |
| 27 | Fwd | gctaacttacattaattgcgttgcgcaGATGGACTGGTACTGTAGATTCACC |
| | | (SEQ ID NO: 262) |
| | Rev | ccaaggggttatgctagttattgcgCAAAGACGCAGAGGCCATCAG |
| | | (SEQ ID NO: 263) |
| 28 | Fwd | gctaacttacattaattgcgttgcgcaATAGAACGATGAAGGATGGAAGCTAC |
| | | (SEQ ID NO: 264) |
| | Rev | ccaaggggttatgctagttattgcgTTGTATTTTGTTGTGTATGGGCGG |
| | | (SEQ ID NO: 265) |
| 29 | Fwd | gctaacttacattaattgcgttgcgcaCGTGATTCAGTTCGCCAGAC |
| | | (SEQ ID NO: 266) |
| | Rev | ccaaggggttatgctagttattgcgCACTCGAAATGGATACCCTGAG |
| | | (SEQ ID NO: 267) |

TABLE 12
Protein accession numbers of defense system
components (proposed gene names underlined).

| # | Gene | Name | Protein Accession |
|---|---|---|---|
| BREX | A | brxA | WP_085962535.1* |
| type I | B | brxB | WP_000566901.1 |
| | C | brxC | WP_001019648.1 |
| | D | pglX | WP_021524842.1 |
| | E | pglZ | WP_001180895.1 |
| | F | brxL | WP_001193074.1 |
| Druantia | A | druA | WP_000549798.1 |
| type I | B | druB | WP_001315973.1 |
| | C | druC | WP_021520530.1 |
| | D | druD | WP_000455180.1 |
| | E | druE | WP_000608843.1 |
| RT-Abi-P2 | A | — | WP_047657908.1 |
| 1 | A | — | WP_005025120.1* |
| 2 | A | Ec67 | WP_000169432.1 |
| 3 | A | — | WP_001034589.1 |
| | B | Ec86 | WP_001320043.1 |
| 4 | A | Ec78 | WP_001549208.1 |
| | B | ptuA | WP_001549209.1 |
| | C | ptuB | WP_001549210.1 |
| 5 | A | drt1a | WP_115196278.1 |
| | B | drt1b | WP_040189938.1 |
| 6 | A | drt2 | WP_012737279.1 |
| 7 | A | drt3a | WP_087902017.1 |
| | B | drt3b | WP_062891751.1 |
| 8 | A | drt4 | GCK53192.1 |
| 9 | A | drt5 | WP_001524904.1 |
| 10.A | A | rdrA | WP_012906049.1 |
| | B | rdrB | WP_012906048.1 |
| 10.B | A | rdrA | WP_155731552.1 |
| | B | rdrB | WP_064360593.1 |
| | C | rdrD | WP_064360592.1 |
| 11 | A | apeA | WP_000706972.1 |
| 12 | A | avs1a | WP_023654314.1 |
| | B | avs1b | WP_084007836.1* |
| 12 | C | avs1c | WP_023654316.1 |
| 13 | A | avs2 | WP_063118745.1 |
| 14 | A | avs3a | WP_126523998.1 |
| | B | avs3b | WP_126523997.1* |
| 15 | A | avs4 | WP_044068927.1 |
| 16 | A | avs5 | WP_001515187.1 |
| 17 | A | dsr1 | WP_029488749.1 |
| 18 | A | dsr2 | WP_015387030.1* |
| 19 | A | — | WP_021577683.1 |
| | B | herA | WP_021577682.1 |
| 20 | A | — | WP_016239654.1 |
| | B | herA | WP_016239655.1 |
| 21 | A | tmn | WP_001683567.1 |
| 22 | A | qatA | STG85056.1 |
| | B | qatB | STG85057.1 |
| | C | qatC | STG85058.1 |
| | D | qatD | STG85059.1 |
| 23 | A | hhe | WP_032200272.1 |
| 24 | A | mzaA | VEA06816.1* |
| | B | mzaB | VEA06814.1 |
| | C | mzaC | VEA06812.1 |
| | D | mzaD | VEA06810.1 |
| | E | mzaE | VEA06808.1 |
| 25 | A | terY | WP_115257868.1 |
| | B | — | WP_115257869.1 |
| | C | — | WP_115257870.1 |
| 26 | A | upx | WP_060647174.1 |
| 27 | A | ppl | STM52149.1 |
| 28 | A | ietA | WP_000385105.1 |
| | B | ietS | WP_001551050.1 |
| 29 | A | — | WP_000860009.1 |
| | B | — | WP_001044652.1 |
| | C | — | WP_001207938.1 |
| | D | — | WP_000985714.1 |

*Probable error in annotated protein start position corrected.

TABLE 13
Predicted protein domains within validated defense systems and controls. Transmembrane
helices were predicted using TMHMM, and all other domains were predicted using HHpred.

| | | | | Representative | | | |
|---|---|---|---|---|---|---|---|
| ID | Gene | Residues | Domain | HHpred Hit | Probability | Start | End |
| BREX | A | 201 | DUF1819 | PF08849.11 | 100 | 6 | 189 |
| type I | B | 200 | DUF1788 | PF08747.11 | 100 | 65 | 187 |
| | C | 1213 | ATPase | PF07693.14 | 96.66 | 43 | 348 |
| | | | DUF499 | PF04465.12 | 99.88 | 247 | 846 |
| | D | 1201 | Methyltransferase | PF02384.16 | 99.7 | 210 | 622 |
| | E | 865 | PglZ | PF08665.12 | 99.12 | 474 | 650 |
| | F | 694 | Lon protease | PF13337.6 | 100 | 30 | 484 |
| | | | Lon protease | PF05362.13 | 99.9 | 486 | 693 |
| Druantia | A | 404 | DUF4338 | PF14236.6 | 99.92 | 45 | 339 |
| type I | B | 548 | CoiA | PF06054.11 | 99.77 | 1 | 182 |
| | C | 627 | Macoilin | PF09726.9 | 96.72 | 167 | 323 |
| | D | 347 | (none) | — | — | — | — |
| | E | 1836 | Helicase | PF00270.29 | 98.45 | 99 | 388 |
| | | | Helicase | 5V9X_A | 97.55 | 1071 | 1208 |
| | | | DUF1998 | PF09369.10 | 98.92 | 1626 | 1710 |
| RT-Abi-P2 | A | 515 | RT | PF00078.27 | 99.09 | 68 | 291 |
| 1 | A | 542 | RT | PF00078.27 | 99.43 | 105 | 309 |
| | | | TIR | PF13676.6 | 97.91 | 411 | 536 |
| 2 | A | 586 | RT | PF00078.27 | 99.45 | 48 | 262 |
| | | | TOPRIM | cd01026 | 96.88 | 367 | 465 |
| 3 | A | 307 | Nuc_deoxy | PF15891.5 | 96.04 | 29 | 128 |
| | B | 320 | RT | PF00078.27 | 99.52 | 53 | 248 |
| 4 | A | 311 | RT | PF00078.27 | 99.37 | 34 | 241 |
| | B | 550 | ATPase | PF13175.6 | 99.8 | 64 | 432 |
| | C | 216 | HNH | PF01844.23 | 97.57 | 43 | 85 |
| 5 | A | 1232 | RT | PF00078.27 | 99.06 | 80 | 382 |
| | | | Nitrilase | PF00795.22 | 98.89 | 953 | 1216 |
| | B | 144 | Transmembrane | — | — | 4 | 26 |
| 6 | A | 425 | RT | PF00078.27 | 99.63 | 54 | 328 |
| 7 | A | 398 | RT | PF00078.27 | 99.39 | 53 | 251 |
| | B | 667 | RT | PF00078.27 | 98.96 | 63 | 323 |
| 8 | A | 540 | RT | PF00078.27 | 99.12 | 67 | 296 |
| 9 | A | 494 | RT | PF00078.27 | 99.14 | 59 | 263 |
| 10.A | A | 851 | ATPase | PF07693.14 | 99.6 | 33 | 364 |
| | B | 856 | Adenosine | PF00962.22 | 99.52 | 166 | 831 |
| | | | deaminase | | | | |
| 10.B | A | 907 | ATPase | PF07693.14 | 99.48 | 29 | 349 |
| | B | 914 | Adenosine | PF00962.22 | 97.63 | 789 | 901 |
| | | | deaminase | | | | |
| | C | 245 | SLATT | PF18183.1 | 96.01 | 120 | 241 |
| | | | Transmembrane | — | — | 44 | 63 |
| | | | Transmembrane | — | — | 78 | 100 |
| | | | Transmembrane | — | — | 127 | 146 |
| | | | Transmembrane | — | — | 151 | 168 |
| 11 | A | 601 | HEPN | PF18739.1 | 86.57 | 507 | 532 |
| 12 | A | 386 | MBL-fold hydrolase | PF00753.27 | 98.79 | 8 | 324 |
| | B | 1935 | Protease | PF02122.15 | 98.23 | 2 | 187 |
| | | | ATPase | PF14516.6 | 99.36 | 204 | 535 |
| | C | 93 | (none) | — | — | — | — |
| 13 | A | 1484 | ATPase | PF14516.6 | 98.93 | 316 | 643 |
| 14 | A | 2092 | DUF4297 | PF14130.6 | 98.41 | 8 | 223 |
| | | | ATPase | PF14516.6 | 99.44 | 250 | 597 |
| | B | 207 | (none) | — | — | — | — |
| 15 | A | 1587 | Mrr | PF13156.6 | 97.05 | 17 | 162 |
| | | | ATPase | PF14516.6 | 99.07 | 204 | 476 |
| 16 | A | 769 | SIR2 | cd00296 | 99.26 | 22 | 244 |
| | | | ATPase | PF14516.6 | 97.6 | 312 | 464 |
| 17 | A | 1275 | SIR2 | cd00296 | 99.44 | 21 | 253 |
| | | | DUF4020 | PF13212.6 | 98.39 | 1114 | 1268 |
| 18 | A | 1207 | SIR2 | cd00296 | 99.47 | 21 | 240 |
| 19 | A | 415 | SIR2 | cd00296 | 99.59 | 26 | 338 |
| | B | 610 | HerA helicase | 4D2I_B | 100 | 10 | 608 |
| 20 | A | 394 | DUF4297 | PF14130.6 | 99.05 | 1 | 191 |
| | B | 571 | HerA helicase | 4D2I_B | 100 | 7 | 568 |
| 21 | A | 1273 | ATPase | PF07693.14 | 97.62 | 39 | 390 |
| | | | Transmembrane | — | — | 160 | 177 |
| | | | Transmembrane | — | — | 199 | 218 |
| 22 | A | 643 | ATPase | PF07693.14 | 99.8 | 15 | 385 |
| | B | 274 | (none) | — | — | — | — |
| | C | 457 | QueC | PF06508.13 | 99.67 | 150 | 369 |
| | D | 263 | TatD DNase | PF01026.21 | 99.94 | 13 | 254 |
| 23 | A | 1911 | DUF4011 | PF13195.6 | 99.81 | 33 | 308 |
| | | | ATPase | PF13086.6 | 97.93 | 427 | 552 |
| | | | Helicase | PF01443.18 | 97.82 | 1379 | 1636 |
| | | | Endonuclease | PF18741.1 | 98.7 | 1683 | 1780 |
| | | | DUF3320 | PF11784.8 | 98.1 | 1841 | 1885 |
| 24 | A | 679 | Ankyrin repeat | COG0666 | 99.52 | 10 | 188 |
| | | | Sigma | COG1191 | 99.81 | 411 | 657 |
| | B | 500 | MutL | COG0323 | 99.81 | 1 | 352 |
| | C | 952 | ATPase | PF13872.6 | 97.51 | 117 | 349 |
| | | | Z1 | PF10593.9 | 100 | 437 | 672 |
| | D | 342 | DUF4420 | PF14390.6 | 100 | 9 | 317 |
| | E | 601 | AIPR | PF10592.9 | 100 | 245 | 562 |
| 25 | A | 277 | vWA | PF00092.28 | 98.93 | 14 | 203 |
| | B | 239 | Phosphatase | PF00481.21 | 99.74 | 5 | 232 |
| | C | 561 | Kinase | PF00069.25 | 100 | 34 | 296 |
| | | | ssDNA-binding | PF01336.25 | 96.18 | 344 | 435 |
| 26 | A | 1272 | DUF1887 | PF09002.11 | 92.5 | 1105 | 1272 |
| 27 | A | 891 | PHP | cd07436 | 99.36 | 4 | 238 |
| | | | ATPase | PF13166.6 | 99.74 | 266 | 836 |
| 28 | A | 384 | ATPase | PF13654.6 | 97.36 | 5 | 349 |
| | B | 754 | Protease | PF00082.22 | 99.87 | 264 | 561 |
| 29 | A | 1022 | ATPase | PF07693.14 | 96.47 | 49 | 312 |
| | | | DUF499 | PF04465.12 | 100 | 79 | 745 |
| | B | 195 | DUF3780 | PF12635.7 | 100 | 1 | 187 |
| | C | 945 | DUF1156 | PF06634.12 | 99 | 18 | 81 |
| | | | Methyltransferase | PF01555.18 | 96.08 | 150 | 202 |
| | | | Methyltransferase | PF01555.18 | 97.76 | 548 | 682 |
| | D | 907 | PLD | cd09179 | 99.17 | 4 | 177 |
| | | | Helicase | 6BOG_B | 100 | 218 | 865 |

TABLE 14
Sequence of vector backbone. Inserts were cloned
between the HindIII and EcoRI
restriction sites (underlined).

CCCGCGCCCACCGGAAGGAGCTGACTGGGTTGAAGGCTCTCAAGGGCATC
GGTCGAGATCCCGGTGCCTAATGAGTGAGCTAACTTACATTAATTGCGTT
GCGCAAGCTTCTGCAGAATTCGCAATAACTAGCATAACCCCTTGGGGCCT
CTAAACGGGTCTTGAGGGGTTTTTTGCTGAAACCTCAGGCATTTGAGAAG
CACACGGTCACACTGCTTCCGGTAGTCAATAAACCGGTAAACCAGCAATA
GACATAAGCGGCTATTTAACGACCCTGCCCTGAACCGACGACCGGGTCGA
ATTTGCTTTCGAATTTCTGCCATTCATCCGCTTATTATCACTTATTCAGG
CGTAGCACCAGGCGTTTAAGGGCACCAATAACTGCCTTAAAAAAATTACG
CCCCGCCCTGCCACTCATCGCAATACTGTTGTAATTCATTTAACATTCTG
CCGACATGGAAGCCATCACAGACGGCATGATGAACCTGAATCGCCAGCGG
CATCAGCACCTTGTCGCCTTGCGTATAATATTTGCCCATAGTGAAAACGG
GGGCGAAGAAGTTGTCCATATTGGCCACGTTTAAATCAAAACTGGTGAAA
CTCACCCAGGGATTGGCTGAGACGAAAAACATATTCTCAATAAACCCTTT
AGGGAAATAGGCCAGGTTTTCACCGTAACACGCCACATCTTGCGAATATA
TGTGTAGAAACTGCCGGAAATCGTCGTGGTATTCACTCCAGAGCGATGAA
AACGTTTCAGTTTGCTCATGGAAAACGGTGTAACAAGGGTGAACACTATC
CCATATCACCAGCTCACCGTCTTTCATCGCCATACGGAACTCTGGATGAG
CATTCATCAGGCGGGCAAGAATGTGAATAAAGGCCGGATAAAACTTGTGC
TTATTTTTCTTTACGGTCTTTAAAAAGGCCGTAATATCCAGCTGAACGGT
CTGGTTATAGGTACATTGAGCAACTGACTGAAATGCCTCAAAATGTTCTT
TACGATGCCATTGGGATATATCAACGGTGGTATATCCAGTGATTTTTTTC
TCCATTTTAGCTTCCTTAGCTCCTGAAAATCTCGATAACTCAAAAAATAC
GCCCGGTAGTGATCTTATTTCATTATGGTGAAAGTTGGAACCTCTTACGT
GCCGATCAACGTCTCATTTTCGCCAAAAGTTGGCCCAGGGCTTCCCGGTA
TCAACAGGGACACCAGGATTTATTTATTCTGCGAAGTGATCTTCCGTCAC
AGGTATTTATTCGGCGCAAAGTGCGTCGGGTGATGCTGCCAACTTACTGA
TTTAGTGTATGATGGTGTTTTTGAGGTGCTCCAGTGGCTTCTGTTTCTAT
CAGCTGTCCCTCCTGTTCAGCTACTGACGGGGTGGTGCGTAACGGCAAAA
GCACCGCCGGACATCAGCGCTAGCGGAGTGTATACTGGCTTACTATGTTG
GCACTGATGAGGGTGTCAGTGAAGTGCTTCATGTGGCAGGAGAAAAAAGG
CTGCACCGGTGCGTCAGCAGAATATGTGATACAGGATATATTCCGCTTCC
TCGCTCACTGACTCGCTACGCTCGGTCGTTCGACTGCGGCGAGCGGAAAT
GGCTTACGAACGGGGCGGAGATTTCCTGGAAGATGCCAGGAAGATACTTA
ACAGGGAAGTGAGAGGGCCGCGGCAAAGCCGTTTTTCCATAGGCTCCGCC
CCCCTGACAAGCATCACGAAATCTGACGCTCAAATCAGTGGTGGCGAAAC
CCGACAGGACTATAAAGATACCAGGCGTTTCCCCCTGGCGGCTCCCTCGT
GCGCTCTCCTGTTCCTGCCTTTCGGTTTACCGGTGTCATTCCGCTGTTAT
GGCCGCGTTTGTCTCATTCCACGCCTGACACTCAGTTCCGGGTAGGCAGT
TCGCTCCAAGCTGGACTGTATGCACGAACCCCCCGTTCAGTCCGACCGCT
GCGCCTTATCCGGTAACTATCGTCTTGAGTCCAACCCGGAAAGACATGCA
AAAGCACCACTGGCAGCAGCCACTGGTAATTGATTTAGAGGAGTTAGTCT
TGAAGTCATGCGCCGGTTAAGGCTAAACTGAAAGGACAAGTTTTGGTGAC
TGCGCTCCTCCAAGCCAGTTACCTCGGTTCAAAGAGTTGGTAGCTCAGAG
AACCTTCGAAAAACCGCCCTGCAAGGCGGTTTTTTCGTTTTCAGAGCAAG
AGATTACGCGCAGACCAAAACGATCTCAAGAAGATCATCTTATTAATCAG
ATAAAATATTTCTAGATTTCAGTGCAATTTATCTCTTCAAATGTAGCACC
TGAAGTCAGCCCCATACGATATAAGTTGTAATTCTCATGTTAGTCATGC
(SEQ ID NO: 268)

TABLE 15-A
Sequences of validated defense systems (sequences shown in Tables 15-B and C)

| Row | | | | Source | | | | Gene | | |
|---|---|---|---|---|---|---|---|---|---|---|
| No. | # | Name | Description | Organism | Strain | bp | Gene | Name | Accession | Residues |
| 1 | Control | BREX type I | — | Escherichia coli | DSM5212 | 13703 | A | brxA | WP_085962535.1* | 201 |
| 2 | | | | | | | B | brxB | WP_000566901.1 | 200 |
| 3 | | | | | | | C | brxC | WP_001019648.1 | 1213 |
| 4 | | | | | | | D | pglX | WP_021524842.1 | 1201 |
| 5 | | | | | | | E | pglZ | WP_001180895.1 | 865 |
| 6 | | | | | | | F | brxL | WP_001193074.1 | 694 |
| 7 | Control | Druantia type I | — | Escherichia coli | DSM5212 | 11823 | A | druA | WP_000549798.1 | 404 |
| 8 | | | | | | | B | druB | WP_001315973.1 | 548 |
| 9 | | | | | | | C | druC | WP_021520530.1 | 627 |
| 10 | | | | | | | D | druD | WP_000455180.1 | 347 |
| 11 | | | | | | | E | druE | WP_000608843.1 | 1836 |
| 12 | Control | RT-Abi-P2 | — | Escherichia coli | ECOR30 | 1921 | A | | WP_047657908.1 | 515 |
| 13 | 1 | — | Retron-TIR | Shigella | NCTC2966 | 2064 | A | | WP_005025120.1* | 542 |
| | | | | dysenteriae | | | | | | |
| 14 | 2 | Ec67 | Retron-TOPRIM | Escherichia coli | NCTC8623 | 2038 | A | Ec67 | WP_000169432.1 | 586 |
| 15 | 3 | Ec86 | Nuc_deoxy + | Escherichia coli | BL21 | 2188 | A | | WP_001034589.1 | 307 |
| 16 | | | retron | | | | B | Ec86 | WP_001320043.1 | 320 |
| 17 | 4 | Ec78 | Retron + ATPase + | Escherichia coli | ECONIH5 | 3551 | A | Ec78 | WP_001549208.1 | 311 |
| 18 | | | HNH | | | | B | ptuA | WP_001549209.1 | 550 |
| 19 | | | | | | | C | ptuB | WP_001549210.1 | 216 |
| 20 | 5 | DRT type 1 | RT-nitrilase (UG1) | Klebsiella | NCTC9143 | 4451 | A | drt1a | WP_115196278.1 | 1232 |
| 21 | | | | pneumoniae | | | B | drt1b | WP_040189938.1 | 144 |
| 22 | 6 | DRT type 2 | RT (UG2) | Salmonella | NCTC8273 | 1780 | A | drt2 | WP_012737279.1 | 425 |
| | | | | enterica | | | | | | |
| 23 | 7 | DRT type 3 | RT (UG3) + RT | Escherichia coli | ECOR12 | 4995 | A | drt3a | WP_087902017.1 | 398 |
| 24 | | | (UG8) | | | | B | drt3b | WP_062891751.1 | 667 |
| 25 | 8 | DRT type 4 | RT (UG15) | Escherichia coli | 21-C8-A | 1838 | A | drt4 | GCK53192.1 | 540 |
| 26 | 9 | DRT type 5 | RT (UG16) | Escherichia coli | KTE25 | 1608 | A | drt5 | WP_001524904.1 | 494 |
| 27 | 10.A | RADAR | ATPase + | Citrobacter | DBS100 | 5526 | A | rdrA | WP_012906049.1 | 851 |
| 28 | | | deaminase | rodentium | | | B | rdrB | WP_012906048.1 | 856 |
| 29 | 10.B | RADAR | ATPase + | Pluralibacter | ATCC33028 | 6689 | A | rdrA | WP_155731552.1 | 907 |
| 30 | | | deaminase | gergoviae | | | B | rdrB | WP_064360593.1 | 914 |
| 31 | | | | | | | C | rdrD | WP_064360592.1 | 245 |
| 32 | 11 | apeA | ApeA (HEPN) | Escherichia coli | NCTC8008 | 1981 | A | apeA | WP_000706972.1 | 601 |
| 33 | 12 | AVAST | MBL + protease- | Erwinia | CFBP5888 | 7246 | A | avs1a | WP_023654314.1 | 386 |
| 34 | | type 1 | STAND | piriflorinigrans | | | B | avs1b | WP_084007836.1* | 1935 |
| 35 | | | | | | | C | avs1c | WP_023654316.1 | 93 |
| 36 | 13 | AVAST | STAND | Escherichia coli | NCTC9087 | 5109 | A | avs2 | WP_063118745.1 | 1484 |
| | | type 2 | | | | | | | | |
| 37 | 14 | AVAST | DUF4297-STAND | Salmonella | NCTC13175 | 7175 | A | avs3a | WP_126523998.1 | 2092 |
| 38 | | type 3 | | enterica | | | B | avs3b | WP_126523997.1* | 207 |
| 39 | 15 | AVAST | Mrr-STAND | Escherichia coli | NCTC11132 | 4964 | A | avs4 | WP_044068927.1 | 1587 |
| | | type 4 | | | | | | | | |
| 40 | 16 | AVAST | SIR2-STAND | Escherichia coli | NCTC13384 | 3411 | A | avs5 | WP_001515187.1 | 769 |
| | | type 5 | | | | | | | | |
| 41 | 17 | dsr1 | SIR2-DUF4020 | Escherichia coli | NCTC9112 | 4212 | A | dsr1 | WP_029488749.1 | 1275 |
| 42 | 18 | dsr2 | SIR2 | Cronobacter | NCTC8155 | 4329 | A | dsr2 | WP_015387030.1* | 1207 |
| | | | | sakazakii | | | | | | |
| 43 | 19 | — | SIR2 + HerA | Escherichia coli | NCTC11129 | 3308 | A | | WP_021577683.1 | 415 |
| 44 | | | | | | | B | herA | WP_021577682.1 | 610 |
| 45 | 20 | — | DUF4297 + HerA | Escherichia coli | NCTC11131 | 3419 | A | | WP_016239654.1 | 394 |
| 46 | | | | | | | B | herA | WP_016239655.1 | 571 |
| 47 | 21 | tmn | Transmembrane | Escherichia coli | ECOR25 | 4415 | A | tmn | WP_001683567.1 | 1273 |
| | | | ATPase | | | | | | | |
| 48 | 22 | qatABCD | ATPase + QueC + | Escherichia coli | NCTC9009 | 5408 | A | qatA | STG85056.1 | 643 |
| 49 | | | TatD | | | | B | qatB | STG85057.1 | 274 |
| 50 | | | | | | | C | qatC | STG85058.1 | 457 |
| 51 | | | | | | | D | qatD | STG85059.1 | 263 |
| 52 | 23 | hhe | DUF4011-helicase- | Escherichia coli | ATCC43886 | 5958 | A | hhe | WP_032200272.1 | 1911 |
| | | | Vsr | | | | | | | |
| 53 | 24 | mzaABCDE | MutL + Z1 + | Salmonella | NCTC5773 | 9416 | A | mzaA | VEA06816.1* | 679 |
| 54 | | | DUF + AIPR | enterica | | | B | mzaB | VEA06814.1 | 500 |
| 55 | | | | | | | C | mzaC | VEA06812.1 | 952 |
| 56 | | | | | | | D | mzaD | VEA06810.1 | 342 |
| 57 | | | | | | | E | mzaE | VEA06808.1 | 601 |
| 58 | 25 | TerY-P | vWA + PP2C + | Citrobacter | NCTC9094 | 3605 | A | terY | WP_115257868.1 | 277 |
| 59 | | | STK-OB | gillenii | | | B | | WP_115257869.1 | 239 |
| 60 | | | | | | | C | | WP_115257870.1 | 561 |
| 61 | 26 | upx | DUF1887 | Salmonella | NCTC6026 | 4100 | A | upx | WP_060647174.1 | 1272 |
| | | | | enterica | | | | | | |
| 62 | 27 | ppl | Phosphoesterase- | Escherichia coli | NCTC8620 | 3066 | A | ppl | STM52149.1 | 891 |
| | | | ATPase | | | | | | | |
| 63 | 28 | ietAS | ATPase + protease | Escherichia coli | ECOR52 | 3676 | A | ietA | WP_000385105.1 | 384 |
| 64 | | | | | | | B | ietS | WP_001551050.1 | 754 |
| 65 | 29 | — | Restriction-like | Escherichia coli | ECOR58 | 9809 | A | | WP_000860009.1 | 1022 |
| 66 | | | system | | | | B | | WP_001044652.1 | 195 |
| 67 | | | | | | | C | | WP_001207938.1 | 945 |
| 68 | | | | | | | D | | WP_000985714.1 | 907 |

*Probable error in annotated protein start position corrected.

TABLE 15-B
Sequences of validated defense systems
(Cloned sequences corresponding to row No. 1-68 in Table 15-A)

| Row No. | | Cloned Sequence |
|---|---|---|
| 1 | Control | acagcaccacgttcatcttccttttttaactgattttacagagactttaatacagttaaaatttta |
| 2 | | tttcctgagctgtaatcgattaagttgatgcatttaatgggaatgatatagggtcatttccagtct |
| 3 | | cacttatagaaatggctaaagcatgactctcgccaaaaccgtttatgtgttgtacataacgcgatc |
| 4 | | atccctctcacaaattgccttttctcatggcatctcgcccggtcccccattacaatcactttttgt |
| 5 | | tttttgcgagctgcattccagtcttcagagggtttttcgatgattaaaaatgacaaggcatggata |
| 6 | | ggagacttgctgggcggaccgctcatgagcagggaaagccgcgtcattgccgaactgttgctaacc |
| | | gatcccgatgaacagacatggcaagagcaaattgttggccacaacattttacaagcctcttctcct |
| | | aacaccgcaaaacgttacgcggcaacaatcaggcttcgcctgaacacgctggataaaagcgcgtgg |
| | | acattgattgccgaaggtagtgaacgggaacgccaacaacttctgtttgtggctctgatgctacat |
| | | tcgccggtagttaaggattttctggctgaagtggtgaacgatctgcgcaggcagttcaaggaaaag |
| | | ttgcctggcaatagctggaacgaatttgtgaatagccaggttcgcctacatccggtactcgccagc |
| | | tactcagattcatctattgcaaaaatgggaaacaatctggtgaaggcgcttgctgaagcgggttat |
| | | gtggatacgccccgcagacgtaacctgcaggcagtttaccttttaccggaaactcaggcagtgtta |
| | | cagcgcctgggacaacaggacttgatatctattctggagggaaaacggtgatagatcccgttcttg |
| | | aatatcgcctgtctcaaatccagagtcgcattaacgaagatcgcttcctcaaaaataacggctccg |
| | | gaaatgaaattggtttttggatctttgattatcccgcgcagtgcgaactgcaggtacgggagcatt |
| | | tgaaatatctgctccggcatctggaaaaggaccataaatttgcctgtctgaatgtcttccaaatca |
| | | tcatcgatatgctcaatgaacgcggccttttcgagcgcgtctgccagcaggaagtcaaagtgggta |
| | | ctgagacgctgaaaaagcagcttgctggtccgttaaatcagaaaaagatcgctgattttatagcga |
| | | aaaaagtcgatctggctgcccaggattttgtcattcttaccggcatgggcaacgcctggccattag |
| | | tacgcggtcatgaactgatgagtgccttgcaggatgtcatggggttcaccccactgctgatgtttt |
| | | atcctggcacctacagcgggtacaacctttccccgctcacagacaccggttcacaaaattattatc |
| | | gcgctttcagactggtaccagatacgggacccgcagcaacattgaatcctcaatgaagagcataac |
| | | aatgaatattgaacagatttttgaaaaacctctaaaacgaaatataaacggggtagtcaaagcaga |
| | | gcaaaccgatgatgccagcgcgtacatcgagttagatgaatatgtcatcacccgcgaactggaaaa |
| | | ccatcttcgccatttcttcgaatcctatgttcctgccactggcccggaacggatccgtatggaaaa |
| | | caagatcggcgtatgggtttcaggcttcttcggttcaggtaaatcgcactttattaagattctttc |
| | | ttatcttttatctaaccgcaaagttacacataacggtacggaacgtaatgcttactccttctttga |
| | | agataaaatcaaagatgcattattccttgccgatattaacaaagcggtgcattacccgactgaagt |
| | | cattctgttcaatattgattcgcgtgccaacgtagatgacaaagaagatgccattcttaaagtctt |
| | | cctgaaagttttcaacgaacgcattggatactgcgctgattttccgcatattgcccatcttgagcg |
| | | cgagctggataaacgcggtcagtatgaaacctttaaagccgcgtttgccgatatcaatggctcgcg |
| | | ctgggaagacgagcgcgacgcttactacttcatcagcgatgacatggcacaagcattaagccaggc |
| | | cacgcagcagagtcttgaatcctcccgccaatgggtggaacaactcgacaaaaacttcccgctgga |
| | | tatcaataatttttgccagtgggtaaaagagtggctggatgacaatggtaagaacatcctctttat |
| | | ggtggatgaagtcggtcagttcattggcaaaaatacgcaaatgatgctgaagctgcagactattac |
| | | tgaaaaccttggggtaatttgcggtggccgcgcatgggttatcgtgacttcgcaggccgatatcaa |
| | | cgcggcaatcggtggtatgagcagtcgcgacggacaggacttctccaagatccaggggcgcttctc |
| | | tacacgcctgcaactttccagctctaacacatcagaagttatccagaaacgtttgttggtaaagac |
| | | tgacgaagcaaaagcggcactggcaaaagtgtggcaagagaaagccgatatcctgcgtaaccagct |
| | | ggcttttgacactacaacaactactgcactacgtccttttaccagcgaagaagagttcgttgacaa |
| | | ctacccgtttgtcccgtggcactatcagattctgcaaaaagtgtttgaatctattcggacgaaagg |
| | | tgcagcgggtaaacaattggccatgggtgagcgttctcagctggaggcattccagacggcggcgca |
| | | gcaaatctcagcgcaagggctggattctctggtgcctttctggcgcttctatgccgccattgagag |
| | | cttcctggaacctgccgttagccgcaccatcactcaggcttgccagaatggcattcttgatgagtt |
| | | cgatggcaacctgcttaaaacgctgttcctgatccgctatgtggaaacgctgaaaagcaccctgga |
| | | taacctggtcacattgtctatcgataggatcgatgccgataaagttgagttgcgccgccgggtcga |
| | | aaaaagtctcaacacgcttgaacgcctgatgctcattgcgcgcgttgaagataaatatgtgttcct |
| | | gaccaacgaagagaaagagatcgaaaacgagatccgtaacgttgatgtcgatttctctgcgatcaa |
| | | caaaaaactggcatcgatcatctttgatgacattctgaaaagccgtaaatatcgttatccggctaa |
| | | caagcaagactttgatatcagccgcttcctgaacgggcatccattagacggcgcagtgcttaacga |
| | | tctggtggtgaagatcctgacccctaaagatccgacttattcgttctataacagcgatgcgacctg |
| | | tcgcccttatacgtcagaaggcgacggctgtattttgattcgtctgcccgaagagggccgtacctg |
| | | gagcgatattgatttagtcgtccagactgaaaagttcctcaaagataacgccgggcaacgtccgga |
| | | acaggcaaccctgctctcagaaaaagcgcgtgaaaacagcaaccgggaaaaattactccgtgttca |
| | | gttggaatcactacttgcagaagcagacgtctgggcgattggcgaacgcttaccgaaaaaatcctc |
| | | cacgccatcgaacattgtcgatgaagcctgccgttacgtgattgaaaacaccttcggcaagctgaa |
| | | gatgctgcggccttttaacggtgacatctcccgtgaaattcatgcattactgacggttgagaacga |
| | | caccgaactggatctcggtaacctcgaagagtccaaccccgacgccatgcgcgaggtagaaacctg |
| | | gatcagcatgaatatcgaatacaataaacctgtgtatttacgcgatattctgaaccattttgcgcg |
| | | tcgcccttatggctggcccgaagacgaagtgaaactgctagtagcccgtctggcctgcaaaggtaa |
| | | attcagcttcagccagcaaaacaacaacgtcgagcgaaaacaggcgtgggagttatttaataacag |
| | | ccgccgccatagcgaattgcgtctgcataaagttcgccgtcatgatgaagcgcaggtgcgtaaagc |
| | | cgcgcaaaccatggctgacatcgctcagcagccgtttaacgaacgggaagagccggcgctggttga |
| | | acatattcgtcaggtatttgaagagtggaagcaagagctgaacgtattccgcgccaaggcagaggg |
| | | cggaaacaatccggggaaaaacgagattgaatccggtctgcgcctgcttaatgccattcttaatga |
| | | gaaagaagattttgccctgatcgaaaaagtctcatcgctgaaagatgaacttctggatttcagcga |
| | | agaccgtgaagatttggtcgacttctaccgtaagcaattcgccacctggcaaaaactgggtgctgc |
| | | gctgaatggcagctttaaatctaaccgcagcgcgctggaaaaagacgccgcagcggttaaagcgct |
| | | gggcgagctggaaagcatctggcaaatgccggaaccttataagcatctcaatcgcatcacgccgtt |
| | | gattgaacaggtccagaacgtcaaccatcagttagtcgaacagcatcgccagcacgccctcgaacg |
| | | cattgacgcccgcattgaggaaagccgtcaacgcttgctggaagcgcacgccacgtcggagctgca |
| | | aaacagcgttctgctgccgatgcaaaaagccagaaaacgcgctgaagtcagccagtcgattccgga |
| | | aattttggcggaacagcaagagacaaaagcgctgcaaatggatgcagataaaaagattaacctgtg |
| | | gatcgacgagctgcgtaaaaagcaagaagcacaactccgggcagcaaatgaagctaaacgcgctgc |
| | | cgactcagaacagacttatgttgtggtggaaaaaaccgttatccaaccggtaccgaaaaaaacgca |
| | | tctggtgaatgtcgccagtgagatgcgtaatgccaccggtggtgaagttctggaaacgaccgaaca |
| | | ggtggaaaaggcgctcgacacgttacgcacaacgctgctggccgtcattaaagcaggcgatcgcat |
| | | tcgccttcagtaactcccatttcagggcagcactctgctgccctttgcaggattttctatgaatac |
| | | caataacattaaaaaatatgccccacaggcccgtaacgacttccgcgatgcggtgatccagaagct |
| | | aacgacgcttgggatcgctgcagataaaaaaggcaatttgcagattgccgaggccgaaaccattgg |
| | | cgagaccgtgcgttacggtcagtttgattacccgttatcgacccttccccgccgcgaacggctggt |
| | | aaaacgcgcccgtgagcagggttttgaggtgctggttgagcactgcgcctacacctggtttaaccg |
| | | cttatgtgcaattcgctatatggagctacacggttatcttgagcacggcttccgtatgttgtccca |
| | | cccggagacgccgaccgcgtttgaggtgctggatcatgtgccggaagtggcagaagccctgctgcc |
| | | ggaaaataaggcgcagctggttgaaatgaagcMccggtaatcaggacgaagccctgtaccgcgaac |
| | | tgctgctggggcagtgccacgccctgcaccacgcgatgccgttcctgtttgaagcggtagatgacg |
| | | aagcggaactgctgttgccggataacctgacccgtaccgactctattctgcgtgggctggttgatg |
| | | atattccggaagaagactgggagcaggtagaggttatcggctggctgtatcagttctatatttcgg |
| | | aaaagaaagatgccgtgattggcaaagtggtgaagagcgaagatattcctgccgccacccagctgt |
| | | ttacgccaaactggattgtgcagtatctggtacaaaactccgttggccgccagtggttgcagacct |
| | | acccggactcgccgctgaaagacaaaatggagtactacatcgagcctgcggaacaaacgccggaag |
| | | tgcaggcgcagctggcggcgattaccccagccagcattgaacccgaaagtattaaagtgctcgacc |
| | | cagcctgcggctccggtcatattttgattgaagcctataatgtgctgaaaaatatctacgaagagc |
| | | gtggttatcgcgggcgtgatattccacaactgattctggaaaataatatttttggtcttgatatcg |
| | | acgaccgcgcggcacagctttccggctttgcattattaatgatggcgcgtcaggatgaccgcagaa |
| | | tatttacccgcgatgtacgtctgaatattgtctctttgcaggaaagcctgcatctggatatcgcca |
| | | aactctggcagcaactgaatttccaccagcaggtacaaaccggcagtatgggggatatgtttgctg |
| | | aaaataacgcgttaacccaaactgacagcgcagaatatcagctgctgatgcgcacgctgaaacgct |
| | | ttgtgaatgcaaaaacgctgggctcactgattcaggtgccgcaggaagaagaagcggaactgaagg |
| | | tattcctggacgcgttgtatcgcctggaacaggaaggcgatttccagcagaagacggcggcaaaag |
| | | cgtttattccgtttattcagcaggcgtggattttagcgcagcgatatgatgcggtagtggcgaatc |
| | | cgccgtatatggggggtaattatatggagacagaacttaagaatttcgtctcttcttactaccctc |
| | | aaggaaaggcggatctttattcttcatttatggtcagattacttttacaattaaaagataatcgca |
| | | ctttaagcctaatgaccccctttacttggatgaatttatcatcatttgaagagctccgaaaaatta |
| | | tacttacaaatttcagcattcagtcattagtacagcctgaatatcattcattttttgagtcagctt |
| | | atgtcccaatttgtgcttttagcatttcaaataccccattaagctggaatgcaaaattttttgatt |
| | | tatcagatttttatggagaaaaaaatcaagctccaaattttcagtatgcaattaaaaatgacaata |
| | | aatgtcattggaaatataacagaatcaccacggactttctatgtactcccggatatatcattgctt |
| | | actctctgcctgattctgcgttatcttgcttcaaaacatccaaaaaacttcatgatgtttgcaatc |
| | | taaaacaaggattaattactggtgataatgaaagatacctaagattctggcatgaaatcagctata |
| | | actctttcagtctcaatgaaaaaagaaaaaaaacaaaatggttcccatatcaaaaaggtggtgcat |
| | | accgtaaatggtatggtaataatgattatgttgttgactgggagaatgatggttattccattaaaa |
| | | acttttataatgacaaaggtaaattacgctcacgccctcaaaacatacaattttattgtaaagagg |
| | | gtttaacatggacaagtttaactatttcgtcactatcgatgagatatgtaccaaatggatatattt |
| | | ttgatgcaaaaggacctatgtgttttccgaaatcctctttggatatctggaatattcttggctatg |
| | | cgaatagcaaagtaatagatatatttctcaaacaattagcgcccaccatggattattctcaagggc |
| | | ctgttggaaatgtcccattcaaatttaacgatggtgatttgaacgagataataaaagaactcgtaa |
| | | acattcacaaacgtgactgggatgaaaatgaaacatcttttgagtttaagagagatatgttggttc |
| | | atttttcaagagatattaacactattaagggtagttttacactaaggcaaggggaaaataaaaaag |
| | | cgattaacagaacaaaatttttagaagaaatgaataactctttctttataaattgctttaatctaa |
| | | ctgatattttatctccagaaattgaactaaacaaaatcacgttaacgcatgcaactattgaaattg |
| | | atattcaaaaaataatttcatatgcaataggctgccaaatgggacgttactcccttgatcgcgaag |
| | | gtctggtatacgctcatgaaggcaataatggcttcgccgatcttgtcgccgaaggtgcttataaaa |
| | | gcttcccggctgatagtgacggcattctgccgctaatggatgaagagtggtttgacgatgacgtca |
| | | cctctcgcgtcaaggagtttatccgcaccgtttggggcgaagaatatttgcgcgaaaacctcgatt |
| | | ttatagccgaagttctcaagcccaaaaaaggcgaatctgcgctggagaccattcgtcgctatcttt |
| | | ccacccagttctggaaagatcatctgaaaatgtataaaaagcgtccaatctactggctattcagct |
| | | ccggtaaagagaaagcgtttgagtgcttggtgtatctgcatcgctataacgatgccacgctgtcga |
| | | gaatgcgtaccgaatatgtggtgccgctgctggcgcgttatcaggccaatattgatcgcctgaacg |
| | | atcaacttgatgaggcttctggcggtgaatccacacgtctgaaacgcgaacgcgacagcctgatca |
| | | aaaaattcagcgaactgcgcagctatgacgatcgcctgcgtcactatgctgatatgagaatcagta |
| | | ttgatctcgacgatggcgttaaggttaactacggcaagtttggcgatctgctggcagatgtcaaag |
| | | ccatcaccggcaatgccccagaggtgatctaaaccagacggcacgttctcctgttgccgggttctg |
| | | cccggtggcaaataccaccgggaaacgcgccgctgctgacatttctccacctcacttcatgataaa |
| | | atgcgccaccgtgtcaaaatctccttttcgcgttttggcgctttcttattcatcgtaacaacatgg |
| | | gattgtgaacttgcaaaatcaggactttattgctggccttaaagctaaatttgccgaacatcgcat |
| | | cgttttctggcacgatcccgataaacgttttattgaggaactggaacagctcaagcttgaaagcgt |
| | | cacgctaatcaacatgacccacgagtcacagctggcggtaaaaaaacgcatcgagattgatgagcc |
| | | agaacagcagttcctgctgtggttcccccatgatgcgccgcctcatgaacaagactggctgctgga |
| | | tatccgcctttacagcagcgaattccatgccgattttgccgccatcaccctgaacacgctgggcat |
| | | tccccagcttggcctgcgcgagcatattcagcgacgcaaggccttcttcagcactaaacgcacgca |
| | | ggcgctgaaaaatctggcgacagaacaggaagatgaagcctcgctggataagaaaatgattgcggt |
| | | gatcgctggcgcaaagaccgcgaaaaccgaagacattttgttcaacctgattacccagtacgttaa |
| | | ccaacaaatagaagacgacagcgaactggaaaacacgcaggcgatgctgaaacgccacggtctgga |
| | | ctcggtattgtgggaaatgctcaaccacgaaatgggctaccaggcagaggagccatcgctggaaaa |
| | | cctgctcctgaaactgttttgtaccgatctctctgcccaggccgacccacagcagcgcgcctggct |
| | | ggaaaaaaatgtcctgctgacgccatccggcagagcatctgccctggcatttatggtgacctggcg |
| | | tgccgatcgtcgctataaagaggcttatgactactgcgctcagcaaatgcaggccgccctgcaccc |
| | | ggaagatcattaccgactcagctcgccgtatgatttgcacgaatgcgaaaccaccctcagcatcga |
| | | acaaaccattattcatgcgctggtaacacagctgctggaagagagcaccacgctcgatcgggaagc |
| | | ctttaaaaaactgctctctgagcgccagagcaaatactggtgtcagacacaaccagagtattacgc |
| | | catctatgacgcattgcgccaggctgagcggttgctgaacctgcgcaatcgccacatcgatggttt |
| | | ccactaccaggacagcgccaccttctggaaagcctactgcgaagaactgttccgcttcgaccaggc |
| | | ttatcgcctgtttaatgaatatgccttgctggttcacagcaaaggagcgatgatcctcaagagcct |
| | | ggatgattatatcgaggcgctctacagcaactggtatctggcagagttaagccgtaactggaacga |
| | | agtgctggaagcggaaaatcgtatgcaggcgtggcaaatccctggcgtgccgcgtcagcagaactt |
| | | cttcaatgaggtggtgaagccacagttccaaaatccgcaaatcaaacgcgtgttcgtgataatttc |
| | | cgatgccctgcgttatgaagtggcggaggagctggggaatcaaatcaataccgagaaacgctttac |
| | | cgcagaactgcgctcgcagctcggcgtgctccccagctacacccaactgggaatggcggcattgct |
| | | gccccatgaacaactttgctatcaacccggtaacggcgacatcgtttatgctgatgggctgtcgac |
| | | ctcgggtattcctaaccgcgataccattctgaagaactataagggaatggcgataaaatcgaagga |
| | | ccttctggagttaaaaaatcaggaagggcgagaccttattcgcgattacgaagtggtgtatatctg |
| | | gcataacacgattgatgccactggcgacacggcatccacggaagataaaaccttcgaagcgtgccg |
| | | cacggcggtggctgaactgaaagatttagtcaccaaggtgatcaaccgcctccacggcacacgcat |
| | | ttttgttacggcggatcacggtttcctgttccagcaacaggcgctttcggttcaggataaaaccac |
| | | tctgcaaattaagccggaaaacaccatcaagaaccacaaacgctttattatcggccatcagcttcc |
| | | cgccgatgatttttgctggaaagggaaagtggcggataccgcaggcgtgagcgacaacagcgagtt |
| | | cctgattccgaaagggatccagcgcttccatttctctggcggcgcgcgcttcgttcatggcggcac |
| | | catgttgcaggaggtttgcgttccggtattgcagataaaagccctgcaaaaaaccgccgcagaaaa |
| | | acagccacagcgccgcccggtggatattgtcgcttaccatccgatgattaagctagtgaacaatat |
| | | cgataaagtgagcctgttgcagacgcatccggtgggcgaactttatgaaccgcgtatcctgaacat |
| | | ttacattgtcgacaacgccaacaatgtggtctcgggcaaagagcgcatcagctttgacagtgataa |
| | | caacaccatggaaaaacgcgtacgcgaagttacgctgaagctgattggcgctaacttcaaccgtcg |
| | | caatgagtactggttgatactggaagacgcacaaacggaaacggggtatcagaagtacccggtcat |
| | | tatcgatctggcgttccaggatgatttcttctaagtgaggcgatatgcaaacccatcatgatttac |
| | | ctgtttcaggcgtatccgcaggggaaattgcctccgagggttacgatctggacgccctgctgaacc |
| | | agcattttgctggtcgcgtggtgcgtaaagatctcaccaagcaactcaaggaaggggcaaacgtcc |
| | | cggtgtatgtgcttgagtatctgctcggcatgtactgcgcctctgacgatgacgatgtggtcgagc |
| | | aagggttgcaaaacgttaagcgtattctggctgataactatgtgcgcccggatgaagcggagaaag |
| | | tgaagtcgctgatccgcgagcgtggttcgtacaaaatcatcgataaagtgtcggtgaaactgaacc |
| | | agaaaaaagacgtttacgaagcccagctttctaacctcggcatcaaagacgcgctggtgccctcgc |
| | | agatggttaaagacaacgagaagctactgacgggcggtatctggtgcatgattaccgtcaactatt |
| | | tctttgaagaagggcagaagacctcacccttctcattgatgacgctcaagcctatccagatgccga |
| | | atatggatatggaagaggtgttcgatgcgcgtaaacactttaaccgtgaccagtggatcgatgtgc |
| | | tgctgcgctcggtgggtatggagcccgccaatattgagcaacgcaccaaatggcaccttatcaccc |
| | | gtatgatcccgttcgtggagaacaactataacgtttgcgagctggggccgcgtggcaccggtaaaa |
| | | gccatgtgtataaagagtgttctcctaactccctgttagtttccggcgggcaaacgaccgttgcca |
| | | acttgttctacaacatggccagtcgccagatcggcctggttggcatgtgggatgtggtagcgttcg |
| | | acgaagtcgcggggatcactttcaaagataaagacggcgtgcaaatcatgaaagattacatggcgt |
| | | caggatctttttctcgcggcagagattcgattgaaggtaaagcgtcgatggttttcgtcggcaaca |
| | | tcaatcaaagcgtagagactctcgttaaaaccagccatttgctggcaccatttccgactgcgatga |
| | | ttgatacagcatttttcgaccgctttcatgcctatattcccggttgggaaatccccaaaatgcgcc |
| | | cggaattctttaccaaccgttacgggctgattacggattatctcgctgaatatatgcgcgaaatgc |
| | | gcaaacgcagtttctctgatgcgattgataaattctttaagctgggtaacaacctcaaccagcgtg |
| | | acgttattgccgttcgacgtaccgtgtcggggttgttaaaactcatgcatcccgatggcgcgtaca |
| | | gcaaagaagatgtgcgagtctgcctgacctatgcgatggaagttcgccgccgcgtgaaagagcaac |
| | | ttaaaaaactgggcggtctggagttcttcgatgtgaactttagctacatcgacaacgaaacgctgg |
| | | aagagttttttgtgagcgtaccggaacagggcggcagcgaacttattcctgccggaatgccaaagc |
| | | cgggtgttgtgcatctggtcactcaggcagaaagcggcatgaccgggctgtatcgttttgaaacac |
| | | agatgactgccggtaatggtaagcatagtgtatcgggtctgggttcaaatacctccgcgaaagaag |
| | | ctatccgcgtcggtttcgattacttcaaaggcaatttgaatcgggtaagcgcggccgcgaaattct |
| | | ccgatcatgaatatcaccttcatgtcgttgaactgcataatactggcccaagcaccgcaaccagtc |
| | | ttgctgcgcttatcgctttatgttcgatattgctggcaaaaccggtgcaggaacagatggtggtgt |
| | | tgggcagtatgacgcttggtggggtaattaacccggtgcaggatcttgccgccagtttgcagctcg |
| | | ccttcgacagcggtgcaaaacgggttctgttgccgatgtcctcggctatggatattccaacggttc |
| | | cggcagagttatttaccaagtttcaggtgagtttttactcagacccggttgatgctgtttataagg |
| | | cgctgggtgtgaattaacgtagtaactattttaatgaac(SEQ ID NO: 269) |
| 7 | Control | ggtgaacgtttggttgatagggtagtaaaactagtaatcatcctataattagctatattcgtggtt |
| 8 | | attagattgaaaacagataacattaacaaaatctataaatcgatttgaatgatttttttcatcaat |
| 9 | | actgttgtaagctcctgctatcaaaagttttgcacacaatctataagctcccagaattgcttgtat |
| 10 | | aaatgctatcattggcgctgtcccgatcgagggagcaaggaggggactctcttgtgccatgcgatt |
| 11 | | aatcactggggctctaagtgaaatttagtgggactaaatactaattggaacgtgagataaaaatgc |
| | | acaaatatccctctataatagttaatatcaaccttcgagaagccaaactgaaaaagaaggtacgtg |
| | | agcatttacaatccttgggttttacaagatctgattctggagcgctccaggccccgggaaatacca |
| | | aagatgtaatacgggctcttcatagttctcaacgagctgagcggatatttgcaaaccaaaagttca |
| | | taacgctaagagcggcaaagcttattaaatttttcgcatccggcaatgaggtcattccggataaga |
| | | tttcaccggtacttgaacgtgtaaagtcaggaacctggcaaggagatctctttaggttagcagcat |
| | | taacttggtccgtacctgtttcaagcggatttggaaggcgtctccggtatcttgtatgggatgaaa |
| | | gcaacggaaaattgatagggctgatcgcaattggtgaccctgtgttcaaccttgcagtccgagata |
| | | atttgattgggtgggatactcatgccagaagttcccggcttgttaatttgatggatgcatacgtcc |
| | | tcggtgctcttcccccttataatgccctgctgggaggaaaattaattgcatgtctgcttcgtagcc |
| | | gcgatctttatgatgactttgcaaaggtctatggtgataccgttggagtaatatctcaaaaaaaga |
| | | aacaagcacgtcttttggctattacaacaacatcgtctatggggcgctcatcggtatataaccgtt |
| | | taaagctggatggaattcaatatttaaaatcgattggatatacaggcggttgggggcattttcata |
| | | tacctgatagcttgttcattgaattacgtgattacttacgtgatatggatcacgcttatgcagatc |
| | | attatatgtttggtaatgggcctaactggcgtttacgtacaactaaggcagctttaaatgcactag |
| | | gatttagagataatttgatgaagcatggaattcaacgtgaagtgtttatcagtcagctagcagaaa |
| | | atgcaactagtattctgcaaacaggcaaaggtgaaccagatctaacctctttgctttctgctaaag |
| | | agatagctgagtgtgcgatggcacgatggatggttccacgatcaattcgcaatccagaatatcggc |
| | | tttggaaagcaagagatctatttgattttattagtaatgactcgctaaactttcccccgtttgacg |
| | | agatagcgaaaacagttgtctaatcttaactgaagggggagtaagtgaattacgctattgataagt |
| | | tcaccgggacactgatattagcagctcgagcaacgaaatatgctcaatatgtttgcccagtttgta |
| | | aaaaaggtgttaacctccgtaaagggaaggttatacccccatattttgctcatttgcccggacatg |
| | | gtacgtcagactgtgaaaattttgttcccggaaattctatcattgtcgaaactattaaaactattt |
| | | caaagcgatatatggatttgcgcttattgattcctgtcggaagtaatagtcgagagtggtcattag |
| | | aattagtgttgccaacctgtaatttatgtagagcaaagataacgttagatgtaggaggcagaagcc |
| | | aaacgcttgatatgaggagtatggtaaagagtcgccagattggtgctgaattatcagtaaaatctt |
| | | accgtattgtttcatatagtggtgaaccagatccaaaatttgtaacagaagttgaaagagaatgcc |
| | | caggtttaccttctgagggagcagcagttttcactgctttagggcgtggggcatcgaagggatttc |
| | | cacgagcacaagagttaagatgtactgaaacatttgcctttctttggcgacaccctgttgctccag |
| | | attttcctgatgaattagaaataaaaagtttagctagtaaacagggatggaatttagctcttgtta |
| | | caattcctgaagtcccttctgtggagagtatttcatggctaaaatcttttacataccttcctgttg |
| | | ttcctgccagaacatctattacagcaatttggccgttcctaaatcaaaaaacaagtattaatcatg |
| | | tcgaatgtgtttattctgacacaatattgttgtcaacaaatatggcaccaacatcatcagaaaatg |
| | | ttggaccaactatgtacgcacaaggttcctctttattactttcagcggttggtgttgaaacatcac |
| | | ctgctttcttcattctaaatcctggagaaaatgactttgtgggcgtttctggctcaattgagcagg |
| | | acgtaaacttatttttttctttctataaaaaaaacgtttctgtacccagaaaatatccctcaatag |
| | | atttggtttttactaagaggaataaagaaaagaccatcgtttccttacatcaaagaagatgcattg |
| | | aagttatgatggaagcacgaatgtttggccataaattagaatacatgtctatgccttctggtgttg |
| | | aaggagtggcaagaattcaaagacaaactgaaagtaatgttattaagttagtttctaatgatgaca |
| | | ttgcagctcatgataagagcatgcggttactatctcctgttgcgttatctcaattatctgattgct |
| | | tagcaaacttaacatgtcatgtagaaatagattttttaggtcttggtaaaatatttttacctggtt |
| | | cttctatgctatcattagatgacgggaaatttattgaattatctcctaatcttcgctcacggatat |
| | | taagttttatacttcaaatggggcacaccctccatggttttagtttaaataatgattttttattag |
| | | ttgagaaattagtggatttgcagccggaaccacacttattaccgcattatagagcattggtaaaag |
| | | aagttaagaccaatggatttgaatgtaaccgctttagataaggtgccttcgaatgagttaccaata |
| | | tagccaagaggcaaaggaacggatctctaagttgggacaatccgaaattgttaactttatcaatga |
| | | gatttctccaactttacgacgtaaagcttttggttgtttaccaaaagtaccgggattcagggcagg |
| | | acatcccactgaaattaaagaaaaacagaaaagattgattgggtatatgttccagtcacatccttc |
| | | ctctgaggagagaaaagcatggaaaagtttttctcttttttggcagttttgggctgaagagaaaat |
| | | tgacaaatcatttagtatgattgataatttaggattaaaagaaaactctggctctatttttattag |
| | | agagcttgctaaaaactttcctaaagttgctagagagaatatcgagcgcctgtttatctttagtgg |
| | | gtttgctgatgatccagacgttataaatgcatttaacctttttcctcctgcagttgttcttgcccg |
| | | cgatatcgtgattgatactcttccaattcgtttagatgagcttgaagcacgtattagtttaattgc |
| | | cgataatgttgagaaaaaaaataatcatattaaagaacttgagttaaaaatagatgctttttccga |
| | | acagtttgataattactttaataatgaaaagagcagtttaaaaataattaatgaactacaatcttt |
| | | gataaactcagagactaaacaatctgatattgctaataaagctattgacgagctttatcattttaa |
| | | tgaaaaaaacaaacagctaatattatctcttcaagaaaaattagattttaatgctctggctatgaa |
| | | tgatatttctgagcatgaaaaattgataaaaagtatggctaatgacatttcagaatttaaaaatgc |
| | | attaacgatcttgtgtgataataaaataaagaataacgagttagattatgtcaatgaattaaaaaa |
| | | actcactgaacgaatagatacacttgaaataaacacatctcaagctagcgaagtgagtgtcaccaa |
| | | tagatttacaaaattccatgaaatagcgcactatgaaaattatgagtatattcatcctccgaagac |
| | | atatctaatagaatttctttaaatttacaggctgttggattgacaaaaaattcagcagaaaaattg |
| | | gctagattgacattagctaccttcgtttctggacaaatcattcaattcagtggctctttggcagat |
| | | attatcgcggatgcaattgccattgctattggtgcaccacgttatcacatatggagagttccagtt |
| | | ggtattatttctgacatggatgcttttgattttatagagactatagctgaatcatctcgctgtctc |
| | | cttttgaaaggggccaatctttcagcatttgagatttatggagcggcaattagagatatagttgtt |
| | | caacggcaaatacatccaacaaattatgaccatctggcattgatagctacctggaaacaaggccca |
| | | gctacattccctgatggaggaatgttggccgagttgggacctgttattgatactgatacattaaaa |
| | | atgcgtggtttatcagctactttaccccaattgaaaccaggttgtcttgccaaggataaatggaca |
| | | aatattgatggactacatcttgatagtgttgatgattatgtagatgaattaagagcattactggac |
| | | gaagctggatttgatgggggaactttgtggaagagaatgattcatattttctatacttcactcata |
| | | aggatccctaatggaaattatatttatgatctttattctgtcttgtctttttatactcttacatgg |
| | | gcaaaaattaaaggtggccccgtccaaaagatagaagatattgccaatcgtgaattaaaaaattat |
| | | agtgcaaaaatatcttcttgaggaggtggttaatggagtggagagcagtatcacgagacaaagcac |
| | | tggatatgttatcaactgcattaaattgtcgatttgatgatgaagggttgagaatttcagcagttt |
| | | cagaatgcttaaggagcgtattatatcaatattctatatctgaaacagaagaagctaggcaaactg |
| | | taacctcgcttcgactcactagtgcagtaaggcgaaaattggtacctttatggccagacattgctg |
| | | atattgataatgctatacatccgggcattatgtctatattgaacagcttggctgaattgggtgaca |
| | | tgattaagttagaaggtggtaattggctaacagctcccccacatgcagtacgaattgacaataaga |
| | | tggctgttttttttggtggagagccttcctgtacattttcaacgggcgtggtagctaaatctgctg |
| | | gaagagttcgcttggttgaagaaaaagtgtgtactggaagtgttgaaatctgggatgcaaatgagt |
| | | ggattggtgccccagcagaaggcaatgaagaatggtcatccagactactatctggaactatttccg |
| | | gctttatcgatgcacctggcaatatgagtgaaacgactgcatatgtgcggggaaaatggctccatt |
| | | tgtcagaactttcttttaataaaaagcaaatctacttatgcagaatgtccgttgataatcactttt |
| | | cctattatttaggagaaattgaagctggacgcttatgtagaatgaattcgttagaatcgtctgatg |
| | | atgtcagaagattacgtttttttctcgatacaaaagataattgtccgctaaaggtccgtatcaaaa |
| | | tatctaatgggctagcaagattaagattaaccagaagattaccaagacgagaaacgaaggtactcc |
| | | tgctaggctggagagaatcaggttttgaaaatgaacattcaggaataacacaccatgtattccccg |
| | | aggaaatattacccatagtgcgtagcgcttttgaagggcttggtattatttggattaacgaattca |
| | | cgcgacggaatgaaatatgattaataaaaataaagtaactgaacgttcaggtatacatgataccgt |
| | | gaaaagccttagtgaaaatctgagaaaatacattgaggcacaatatcatatccgggatgaagggtt |
| | | aattgctgagcgacgagcgcttttacagcaaaatgaaactattgctcaagctccttatatagaagc |
| | | aaccccaatttatgaacctggtgcgccatacagtgaattgcctattcccgaagcagcaagtaatgt |
| | | gctaactcaactatcagaacttggaattggcctctatcaacgcccctataaacaccaatcacaggc |
| | | acttgagtcatttcttggcgaaaacgcttctgatctggtcattgcaacaggtacaggctccggtaa |
| | | gactgaaagctttctaatgccaattattggaaaattggcgattgaatcttccgagagacctaaatc |
| | | tgcatcccttccaggttgtagagcaattttattatatccaatgaatgcattagttaacgatcaact |
| | | tgctcgtatcagacgtctttttggtgattctgaagcctctaaaatactgagatctggaagatgtgc |
| | | ccctgtacgctttggcgcttatacgggaagaacgccttaccctggtcgtcgtagctctagacgaga |
| | | cgagctttttatcaaaccccttttcgatgagttttacaataaactcgcaaataacgcccccgtacg |
| | | tgcggaactgaaccgcattggtcgctggccaagtaaagatcttgatgctttttatgggcaaagcgc |
| | | atctcaggctaaaacctacgtctcaggcaaaaaaacgggtaagcaatttgttttgaacaattgggg |
| | | ggagaggctaattacccagcctgaggatcgtgagctaatgacccggcatgaaatacagaatcgctg |
| | | tccagaattactgataacgaactactccatgcttgagtatatgctgatgcgacctatcgagcgtaa |
| | | tatttttgagcagactaaggaatggctcaaagctgatgagatgaatgagcttatcttagtgcttga |
| | | tgaagcgcatatgtatagaggagcagggggagcagaggtagcccttttaatacgtcgcctctgtgc |
| | | tcggttggatattccccgggaacgtatgcgctgcatccttaccagtgctagtctagggtccattga |
| | | ggatggagaacgttttgcccaagacttaactggcttatcaccaacctcttcgaggaaatttcgaat |
| | | tattgagggtacaagggaatcgcgtcctgagtcacaaattgttaccagtaaagaagctaatgcact |
| | | ggctgaattcgacctaaattcatttcagtgcgtagctgaggatcttgaatctgcatatgcagcaat |
| | | agagtctcttgccgaacgaatgggctggcaaaagccgatgataaaagatcatagtacactacgtaa |
| | | ttggttatttgataatttgactggttttggtcctattgaaacgcttattgaaatagtttcaggtaa |
| | | agcggttaagctaaatatcttgagtgaaaacctttttccagactctccacagcaaatcgcagagcg |
| | | agcaacagatgcattactcgcattgggttgctatgctcagagggcatccgatggcagagtgcttat |
| | | tccaactcgcatgcatcttttttatcggggattaccaggtctttatgcctgtatagatcccgattg |
| | | taatcaacgtttgggtaaccatagcgggccaactatacttggccgcctttatacgaaaccactgga |
| | | tcaatgtaaatgcgcttcaaaagggcgagtctacgaattatttacccaccgtgactgcggtgcggc |
| | | ttttattcgtggatacgttagttccgaaatggactttgtatggcaccagccgaacggaccattatc |
| | | agaagatgaggatatcgatcttgttcccatagatatattggtcgaggaaacacctcatgtacatag |
| | | tgattaccaggacagatggctacatatagcaacaggacgcctttctaaacagtgtcaagatgagga |
| | | ttctggttatcgtaaagtctttatacctgaccgagttaagtctggatcagaaattacatttgatga |
| | | atgccctgtttgtatgcgtaagacaagaagtgctcagaatgaaccgtctaaaattatggatcatgt |
| | | tacaaaaggggaagcaccttttacaacgttagtacgtacacagatatctcaccagccagcgagtcg |
| | | tcctattgatggtaaacatcccaatgggggaaaaaaagtacttattttttctgatggccgacaaaa |
| | | agcagctcggcttgcacgtgatattcctagagatattgagcttgatttgtttcggcaatccattgc |
| | | tctcgcctgttctaaactgaaagatatcaatcgggaacccaaaccaacatcagtactttaccttgc |
| | | tttcctatcagtcctttctgaacatgacttgcttatttttgatggggaagattcacgaaaagttgt |
| | | aatggcccgtgatgaattttatcgtgattataatagcgatctggctcaagcttttgatgatagctt |
| | | cagcccccaagagtcaccgtcacgatataaaatagcgttgcttaaacttttatgtagcaattacta |
| | | ttctctttccggaacaacagttggttttgttgaaccatcgcagcttaaatcaaaaaaaatgtggga |
| | | agatgtgcagtccaagaagctcaatattgagagcaaggatgttcatgctttagctgttgcttggat |
| | | tgataccttactcactgaatttgcttttgatgaatctattgattcgacactacgaatcaaagcagc |
| | | tggattctacaaacccacttggggtagtcaaggacggtttggaaaagctcttaggaaaaccctgat |
| | | acagtatcctgctatgggggagctttatgtggaagttttggaggagatttttcgtactcatctgac |
| | | attaggaaaagatggtgtctactttcttgctccaaatgcactacgtctgaaaatagatctcttgca |
| | | tgtctggaaacaatgtaatgactgcacggcactaatgccatttgctttagaacattctacttgcct |
| | | tgcttgtggtagtaacagtgtcaaaacagtcgagccgtcggaaagcagctatattaatgcacgaaa |
| | | aggattctggcgttcgccggtagaagaagttttggtttcaaattcgcggcttctaaaccttagcgt |
| | | tgaagagcatactgctcaactctcacatagagatagggccagcgttcatgccactacagaactcta |
| | | cgaactgagattccaagatgttcttattaatgataacgacaagcccattgatgtacttagttgtac |
| | | gacgacgatggaagtgggggttgatattggatctctggttgctgttgctttaagaaacgtccctcc |
| | | gcaacgagaaaattatcagcaacgtgctgggcgagcaggccgccgtggcgcatctgtttcaacggt |
| | | ggttacatattctcaaaatggccctcatgatagttattatttccttaatcctgaacgcattgttgc |
| | | aggttctcctcgtacacctgaagtgaaagtaaataatcccaaaatagccagaagacacgttcattc |
| | | ttttttagttcagaccttttttcacgagttaatggaacaaggaatttataatcccgcagagaaaac |
| | | tgccatacttgagaaagcacttggtactacacgagatttttttcatggagcaaaagatactggcct |
| | | aaatctcgatagctttaataattgggttaaaaaccgtattctatctactaatggtgatttgagaac |
| | | aagtgttgcagcatggcttcctcctgttcttgaaactggagggctttctgccagtgactggtttgc |
| | | taaggtagcagaggaatttttaaatacactccatgggctggctgaaattgttccacaaactgccgt |
| | | tcttgttgatgaggaaaatgaagatgatgagcagacttctggtggaatgaaatttgcacaagaaga |
| | | attacttgagttcctgttttaccatggtttattaccaagttatgcatttcctacaagcctctgtag |
| | | tttcttggtagaaaaaattgtaaagaatattagaggttcttttgaggtgcgaacagtacaacagcc |
| | | tcagcaatcaatttctcaggctctgagtgaatatgccccgggacgtttgattgttattgataggaa |
| | | aacctatcgctctggtggtgttttttctaatgcattgaaaggcgaactaaaccgggcaagaaagct |
| | | tttcaataatcccaaaaagtttattcattgcgataagtgctcttttgtccgcgatcctcataataa |
| | | tcagaatagcgaaaatacttgtccgatctgtggtggcattctaaaagtagaaataatgattcagcc |
| | | cgaagtctttggacctgaaaatgccaaggaacttaatgaggacgacagagagcaagaaatcaccta |
| | | tgtaacagcggcacaatatccacaacctgttgatcctgaagattttaagttcaataatggaggtgc |
| | | tcatattgtttttactcacgcaatagatcagaaactggtgacggtgaaccgagggaaaaatgaggg |
| | | ggagtccagtggtttttcagtatgttgcgaatgtggtgcggcctccgtttatgattcctactcacc |
| | | ggcaaagggggcacatgaaagaccgtataaatatatagcaactaaggaaacgcctcgcttatgctc |
| | | tggcgagtataaacgcgtttttctcggacatgatttccgtactgatttgcttttattacgaataac |
| | | cgttgggtctccgcttgtaactgatacttcaaatgctatcgttttacggatgtatgaagatgcatt |
| | | atatacaatagcggaagcactaaggcttgcagctagtcgccataaacaactggatcttgatcctgc |
| | | tgagtttggctctggtttcagaattttacccactatagaggaagatactcaggcattggatctctt |
| | | cctttatgatactttatccggcggtgcgggttatgcggaagtagcagcagcgaatctagatgacat |
| | | tcttactgcaacactcgcattgttagaaagctgtgagtgcgatacctcctgtacagattgtctcaa |
| | | tcatttccacaaccagcatatacaaagccgtctcgataggaaactaggtgcatctttacttcgtta |
| | | tgcactatacggaatggttcctcgttgtgcttcacctgatattcaggtagaaaaattgtctcaatt |
| | | gagggcaagtctggaattggatggttttcaatgcataattaagggaactcaggaggcacctatgat |
| | | tgtgagtttgaatgaccgttctattgcagtgggaagttatcctggtcttattgatcgacccgactt |
| | | tcaacacgacgtatataagtcaaagcatactaatgctcatatagcctttaatgaatatcttcttcg |
| | | ttcaaatctgccacaatcgcatcaaaatattagaaaaatgttgcgctgatagcagcagtattgagt |
| | | gccctaaagccctgtagggcactcaaggttttcagtgcgtgagcgggctttaactgaagccataaa |
| | | tgtacgtatgggagaaaatgtgaccatttaactcgccagcaactattgcacaatgtaaaattatgc |
| | | ccattgag (SEQ ID NO: 270) |
| 12 | Control | acatcccgtcatcatgccatcacgacgcgctgagacgctgaaaaaataaaatcagcaccaccgtca |
| | | gcgcgcagtgctttccccgcctcgcccgcccgcttcatgagacggttttaatgcagttgcattatg |
| | | tcccgctcctcagtgctgcgctccatcctgattacaaaaaccgttatcaaaaacacatgcaaatag |
| | | acgcagtcaaatgcgctaccgcctctcgcaataccttcaatttcatgataaaaaacatcatcccta |
| | | acaagagcattatcctcatgaaaaaagtatatgaactaaccagtgaagaagcactgtcatattttc |
| | | ttcgccatgactcctacacaacattagaattaccggcttatattaatttcaccacattattaaatg |
| | | atattaattcatctatccataacaaaaaaattaaaattgaaccaaccgccaaggagctgatgggta |
| | | aagatatcaattatgaggtgcttgtcagtaaagatggtctatatagctggcgtaggataacactta |
| | | tcaatcccctttattatgtctacttctgtagaaaaatcacagcaccagcaacctgggaaatcataa |
| | | cagaaaaattcaaatcttttgaatcaaacgacctttttacatgttcaagcatccccgtcagaaaag |
| | | acaactcgtcaaacattgctgcgtctgtaatgaattggtgggaagattttgaacaaaaaagccttg |
| | | cccttgctcttgaatacgaattcatgttcagcactgacatctcaaacttctacccatcaatatata |
| | | ctcatagttttgaatgggtattcatatcaaaagaagaggcaaagaagaaaaaaagcaaaaataacc |
| | | cagggggattaattgacagccacattcaaatgatgatgaacaaccagacaaatggtattccactcg |
| | | gcagcacattgatggatacatttgctgagcttatcttgggtcaaatcgatatagaattaagaaaaa |
| | | aaactaacgaactcaaaataataaactacaaggtagtacgctaccgtgatgattaccggatcttct |
| | | ctaatagcaaagatgatttagacataatatcaaaatgtttagtcaatgtattgggcgattttggtt |
| | | tagatctaaactcaaaaaaaactgaactatatgaagacatcatacttcattcgttgaaacaagcta |
| | | aaaaagactacatcaaagaaaaaagacataagtcactccagaaaatgctctattcaatatatttat |
| | | tttcacttaaacatccaaactcgaaaacaaccgttagatatctaaatgattttcttaggaatttat |
| | | ttaagcgaaagacaattaaagataacggccaacaggttgatgctatgcttggtattatttcaagca |
| | | tcatggcaaaaaaccctacaacgtacccagtaggaacggcaattttctcaaaactcctcagttttc |
| | | tttatggtgatgacacccaaaaaaaattaacaaagctagaacaactccataaaaaactggataaac |
| | | aacccaatacagaaatgcttgacatatggtttcagcgaactcaagcaaaaataaacctagagtgga |
| | | ataaatcttataagtcagctctatgcgtccgtataaatgatgaactcacaaaagagaaaacatttt |
| | | ctgtaaataatttatggaatattgactggatccaaggaaaagaaacaagccccaataaagccaaaa |
| | | tattatccttgctaagaaaaacaaaaatcgttgacacagataaatttgataaaatggatgacaata |
| | | taacacctgaagaagttaatctattctttaaagagcacagcaattaatatcccaaagccatgttag |
| | | taacataacatggcttttttaaatcactcattatcagttatcaagaacgaacataacattctattc |
| | | cgaggag (SEQ ID NO: 271) |
| 13 | 1 | agttaatgactattgtgagcgagaaacgcgctactactatatatagacagacaagatgcacttact |
| | | gaataaatactcataacggagaaaccagctgtatagtgaacaatagatttccagtagcatattttt |
| | | acttcacttttagttattaatatgataatcataaactacggctctgccttaaatttgtgaggttgt |
| | | ttcgcctcgaaggaactaatgttaggacatacgccaccgttcagtcgatggtaacgcttcttaact |
| | | agtggtccgctaagtgatgcgcaaagtgattgggcagagccgaaacgtttacaatccgataggagt |
| | | tggttttgtcgctacatgataaattattaatgcataacttcgcattagccaataaaaaaagccctg |
| | | acttcatatctgaacttcctcaaattgaacctaaaccatacagcaatggacataaaattaaatgga |
| | | taaaccacacacttactagcactgaagttactccccctgataacctgattaaaatatgcatattga |
| | | ttgagtcaggggaaattgctataacatcagtaagtgatattgccaatttacttggagttcctgctg |
| | | gccaattactttatatactatatcgtaaaaaagataattatcgtacttttgaaatagaaaagaaga |
| | | atggtaaaaaaagagtcattaatgctccttgtggcggtctatcgatactccaaacgagactaaagc |
| | | ccgttcttgaatatttctacaggccaaagaaatctgctcatggttttataaaaggaaagagcatca |
| | | ttactaatgctgggatgcatattaaaaaaaattttgtcgtaaacattgatctagaaaactatttcg |
| | | aatcaataagttttgctagggtttatggaatatttaaaagtaaaccttttaattttgctcatcctg |
| | | cagctactgttttagctcagttatgtactcacaatggaaaattacctcaaggtgcgtgtacatcgc |
| | | caatattagcaaatattgcatcagcttctctagacaaacagctcacccaatttgcaggaagaaaaa |
| | | aaatatcttattctaggtatgctgacgacataactttttctttcaatcagagaaatattgatataa |
| | | tcaaaaaaaacgacgacggaagttatagtcttagtgaaactatagacaatattatttcaaaaaatg |
| | | gctttaaaataaattatgataaatttagagttcaaaccagaaatacaagacaaagtgttactggct |
| | | tagtggttaatgataaagttaacattaacagaagatatataagaattacacgttcaatgattcata |
| | | gatggacagatgataagctaaagtatgcacttctctttgctacagaaaaaggatatcaggcaaagg |
| | | ataataaccacgcaattcaaattttccgaaatcatatttatggaaggcttagctttataaaaatgg |
| | | ttagagggaaagactatccaggatatttaaaactgatgtcatacatgagtcataacgatccattaa |
| | | aaacccaagaaggattgcgagcaatgaaagaaacagaaaactttgatgtttttatatgccatgcaa |
| | | gcgaagacaaaaaagacattgcaattccaatatatgacgagttaactaaacttaaaatttcagcct |
| | | tcatagatcatgttgagataaaatggggcgactccttaattgataaaataaatgcagcactagtta |
| | | aatcaaaatatgtcatcgctattttatctgctaattcagtcaataaggaatggcctcaaaaagaat |
| | | taagagcagttttagccagcgaaatatcgagtggcgacgtaaaacttttgaccttattaaaaaaag |
| | | aagacgaggaggtcgtaaacctatcattacctttacttagtgataagttttatatggtctatgata |
| | | ataatcctgaagtagtcgccaacaatattaaatcactcttacaacgataattctctcacaaaagaa |
| | | aatgtgcagattgatgcgtattaagtattaatctgcacatacaaaaaaaataataaaataatacat |
| | | ttttcataacttgtagg(SEQ ID NO: 272)t |
| 14 | 2 | cgcgctatcacgtaaaataggcaaaatacttctggaaaacagaaagttgaagtgatatgttcataa |
| | | acacgcatgtaggcagatttgttggttgtgaatcgcaaccagtggccttaatggcaggaggaatcg |
| | | cctccctaaaatccttgattcagagctatacggcaggtgtgctgtgcgaaggagtgcctgcatgcg |
| | | tttctccttggccttttttcctctgggatgaagaagaaatgacaaaaacatctaaacttgacgcac |
| | | ttagggctgctacttcacgtgaagacttggctaaaattttagatgttaagttggtatttttaacta |
| | | acgttctatatagaatcggctcggataatcaatacactcaatttacaataccgaagaaaggaaaag |
| | | gggtaaggactatttctgcacctacagaccggttgaaggacatccaacgaagaatatgtgacttac |
| | | tttctgattgtagagatgagatctttgctataaggaaaattagtaacaactattcctttggttttg |
| | | agaggggaaaatcaataatcctaaatgcttataagcatagaggcaaacaaataatattaaatatag |
| | | atcttaaggatttttttgaaagctttaatttcggacgagttagaggatattttctttccaatcagg |
| | | attttttattaaatcctgtggtggcaacgacacttgcaaaagctgcatgctataatggaaccctcc |
| | | cccagggaagtccatgttctcctattatctcaaatctaatttgcaatattatggatatgagattag |
| | | ctaaactggctaaaaaatatggatgtacttatagcagatatgctgatgatataacaatttctacaa |
| | | ataaaaatacatttccgttagaaatggctactgtgcaacctgaaggggttgttttgggaaaagttt |
| | | tggtaaaagaaatagaaaactctggattcgaaataaatgattcaaagactaggcttacgtataaga |
| | | catcaaggcaagaagtaacgggacttacagttaacagaatcgttaatattgatagatgttattata |
| | | aaaaaactcgggcgttggcacatgctttgtatcgtacaggtgaatataaagtgccagatgaaaatg |
| | | gtgttttagtttcaggaggtctggataaacttgaggggatgtttggttttattgatcaagttgata |
| | | agtttaacaatataaagaaaaaactgaacaagcaacctgatagatatgtattgactaatgcgactt |
| | | tgcatggttttaaattaaagttgaatgcgcgagaaaaagcatatagtaaatttatttactataaat |
| | | tttttcatggcaacacctgtcctacgataattacagaagggaagactgatcggatatatttgaagg |
| | | ctgctttgcattctttggagacatcatatcctgagttgtttagagaaaaaacagatagtaaaaaga |
| | | aagaaataaatcttaatatatttaaatctaatgaaaagaccaaatattttttagatctttctgggg |
| | | gaactgcagatctgaaaaaatttgtagagcgttataaaaataattatgcttcttattatggttctg |
| | | ttccaaaacagccagtgattatggttcttgataatgatacaggtccaagcgatttacttaattttc |
| | | tgcgcaataaagttaaaagctgcccagacgatgtaactgaaatgagaaagatgaaatatattcatg |
| | | ttttctataatttatatatagttctcacaccattgagtccttccggcgaacaaacttcaatggagg |
| | | atcttttccctaaagatattttagatatcaagattgatggtaagaaattcaacaaaaataatgatg |
| | | gagactcaaaaacggaatatgggaagcatattttttccatgagggttgttagagataaaaagcgga |
| | | aaatagattttaaggcattttgttgtatttttgatgctataaaagatataaaggaacattataaat |
| | | taatgttaaatagctaatgaacagccctaacgttatgaacgctaaggctgatttttcg |
| | | (SEQ ID NO: 273) |
| 15 | 3 | gctcatgttatgcatgtgcatgaaaaccactgcataaagcgggcaggcgtggcggggatacgagcg |
| 16 | | cgcgccatgtggtatggagattggatctattcataacttgatgtataaagtagaaaaaaaagcggg |
| | | gagattatgaataaaaaatttaccgatgagcagcaacaacagcttataggacatctcacaaagaaa |
| | | ggcttctatcgaggagctaatattaaaataaccatttttctatgtggtggtgacgttgctaatcat |
| | | caatcttggcgtcatcaattatcacaatttttagcaaagttcagtgatgttgatatattttatcca |
| | | gaagatctatttgatgatcttttggctggtcaagggcagcatagccttttaagtttagaaaatatt |
| | | ctggctgaagctgtcgatgtaataattttatttcctgaaagtccggggtctttcacagagcttggt |
| | | gcgttctctaataatgaaaacttaaggagaaagttgatttgcattcaagatgcaaaatttaaatca |
| | | aaacgtagctttattaactatggtcctgttcgcctgttgcgtaagtttaattcaaaatctgttttg |
| | | cgttgtagttcaaatgaactaaaagaaatgtgtgattcatctattgatgttgccagaaaattacga |
| | | ttatataaaaaattaatggcatctattaagaaggttaggaaagaaaataaagtatcaaaagatatt |
| | | ggaaatatattatacgcagagcggtttctattgccttgtatctatttactggatagtgtcaactac |
| | | cgcacactgtgtgaactagcttttaaagcgataaagcaagatgatgttttatctaaaattattgtt |
| | | agatccgttgtttctcgtctaataaatgaacgaaaaatacttcaaatgactgatggttatcaggtc |
| | | actgctttgggggctagctatgttaggagcgtctttgatagaaagacacttgaccgattgcggctt |
| | | gagattatgaattttgaaaaccgtagaaaatcaacatttaactatgataagattccgtatgcgcac |
| | | ccttagcgagaggtttatcattaaggtcaacctctggatgttgtttcggcatcctgcattgaatct |
| | | gagttactgtctgttttccttgttggaacggagagcatcgcctgatgctctccgagccaaccagga |
| | | aacccgttttttctgacgtaagggtgcgcaactttcatgaaatccgctgaatatttgaacactttt |
| | | agattgagaaatctcggcctacctgtcatgaacaatttgcatgacatgtctaaggcgactcgcata |
| | | tctgttgaaacacttcggttgttaatctatacagctgattttcgctataggatctacactgtagaa |
| | | aagaaaggcccagagaagagaatgagaaccatttaccaaccttctcgagaacttaaagccttacaa |
| | | ggatgggttctacgtaacattttagataaactgtcgtcatctcctttttctattggatttgaaaag |
| | | caccaatctattttgaataatgctaccccgcatattggggcaaactttatactgaatattgatttg |
| | | gaggattttttcccaagtttaactgctaacaaagtttttggagtgttccattctcttggttataat |
| | | cgactaatatcttcagttttgacaaaaatatgttgttataaaaatctgctaccacaaggtgctcca |
| | | tcatcacctaaattagctaatctaatatgttctaaacttgattatcgtattcagggttatgcaggt |
| | | agtcggggcttgatatatacgagatatgccgatgatctcaccttatctgcacagtctatgaaaaag |
| | | gttgttaaagcacgtgattttttattttctataatcccaagtgaaggattggttattaactcaaaa |
| | | aaaacttgtattagtgggcctcgtagtcagaggaaagttacaggtttagttatttcacaagagaaa |
| | | gttgggataggtagagaaaaatataaagaaattagagcaaagatacatcatatattttgcggtaag |
| | | tcttctgagatagaacacgttaggggatggttgtcatttattttaagtgtggattcaaaaagccat |
| | | aggagattaataacttatattagcaaattagaaaaaaaatatggaaagaaccctttaaataaagcg |
| | | aagacctaat (SEQ ID NO: 274) |
| 17 | 4 | acgtgtcttgatttaagttgacttcaagactataaagtctcaagtaacagtcggttagcttccttc |
| 18 | | atgggttggtcatgccgggttgttaagtatggctgtttgcgataagctttaaatactctttagcgt |
| 19 | | tggacggttacgtctagtcgggtgattagccagactctaacttattgaacgtattaagggttgcga |
| | | aagtgtcgcaacccgagatcgttcctctctcgggttgcgacactttcgcttcctcaagtaaagagt |
| | | gaagcccggcgcaaatgcgccgggccattttcaggtactgttatgtctgttattcgtggattagct |
| | | gcggttttacgtcaaagtgactccgatatcagcgcctttcttgtaaccgccccgagaaagtacaaa |
| | | gtttacaaaatccctaagcgtacgacgggatttagagtcattgcccagcctgccaaagggctaaaa |
| | | gatatccaacgagcctttgttcagctctatagcctccctgttcatgatgcttcaatggcctatatg |
| | | aaagggaagggaattcgtgataatgctgcagcacatgctggcaaccagtatctcctaaaggcggat |
| | | ctggaggatttttttaactcaattacaccggcaattttttggcgttgcattgaaatgtcatctgcg |
| | | caaacacctcaatttgaacctcaggataagctttttattgaaaagatccttttctggcaaccgata |
| | | aagcgtcgcaaaaccaaattgatattgagtgttggtgcgccttcttcaccagtcatatccaatttc |
| | | tgtatgtatgagttcgataatcgaattcatgcggcttgcaagaaggtggagataacatacacacgc |
| | | tatgcagatgatctcacgttctcgtctaatatccctgatgtactgaaagcagttccttcaacgctt |
| | | gaggtcttactgaaggatttatttggaagcgcgctcagacttaatcacagcaaaacggttttttca |
| | | tcaaaagcacataaccggcatgtgactggtataacaataaataatgaagagacactttcactcggg |
| | | cgcgatagaaaaagatttatcaaacatctgattaaccagtataagtatggactccttgataatgag |
| | | gataaagcttatctgatcgggctgttagcatttgccagccatatcgagcctagtttcatcacacgg |
| | | atgaacgaaaaatactcattagaactcatggaacgcctgagaggacagagatgaccaagcaatatg |
| | | aaagaaaagcaaagggtggaaatttactgtcagcattcgaactttaccaacgtaatagtgataaag |
| | | cgcctggtctgggtgaaatgttagtgggtgagtggttcgaaatgtgcagggattacattcaggatg |
| | | gacatgttgatgagtcaggaatatttcgtccagataatgcgttctatcttcgccgcctgacgttaa |
| | | aggattttcgccgtttctctcttctggaaattaaactcgaagaagatctgacagtcattattggca |
| | | acaatggtaaagggaagacaagtatcttatatgcgattgcaaaaacgctgagttggttcgtcgcga |
| | | acatcctgaaggaaggtggtagtggacaaaggttaagcgaaatgactgacataaaaaatgacgctg |
| | | aagacaggtattcagatgtcagtagcactttcttctttggcaaaggacttaagagtgtgccgatca |
| | | gattgtcacgctcagcccttggtacagccgaaaggcgggacagcgaggttaagcctgccaaggatt |
| | | tagctgatatatggcgagtcatcaatgaggtgaatacgatcaacttgccgacgttcgctctttaca |
| | | acgttgagcgatcgcaaccgtttaaccgcaacataaaagataataccggacgcagagaagagcgct |
| | | ttgatgcctatagtcaaacgctcggtggcgcaggacgtttcgatcatttcgttgagtggtacattt |
| | | acctccataagcgtactgtatcagatatctcaagttctattaaagaacttgaacaacaggttaatg |
| | | acttacagcgtaccgttgatggcggtatggtttcggtaaaatcacttctggaacagatgaagttta |
| | | agcttagtgaagctatagaaagaaatgatgctgcggtttcctcgagagtgttaactgagtctgttc |
| | | aaaaaagtattgttgagaaagcaatctgctcggttgtccctagtatcagcaatatatgggttgaaa |
| | | tgataacgggttctgatttagtcaaagttacaaatgatgggcatgatgttactattgaccaattat |
| | | ctgacgggcagcgtgtatttctgtcgttggtggccgatcttgcgcgaagaatggttatgctgaatc |
| | | ccctgctggaaaatccattagagggacgtggcattgttttaattgatgaaatagaacttcaccttc |
| | | atcctaagtggcagcaggaagttatcctgaacctgcgcagtgcattccctaacattcaatttatta |
| | | ttacaacacacagtcccattgttctttctacaattgagaaacgctgtattcgtgagtttgagccca |
| | | acgatgatggcgaccaatcattccttgattctcccgatatgcaaacaaagggaagtgagaatgctc |
| | | aaattcttgagcaggtaatgaacgtacattctacaccgcctggtattgctgaatctcattggttag |
| | | gtaattttgaactattgcttttagataattctggagaacttgataaccactctcaagtgctttacg |
| | | accaaatcaaggcgcactttggcatcgatagtattgagttgaagaaagcagatagccttattcgca |
| | | ttaataagatgaagaataaactgaacaagataagggccgagaaggggaaatagtaatgagagagtt |
| | | agcccggctggagagaccggagattcttgaccagtatatagccggtcaaaatgactggatggagat |
| | | tgatcagtctgcggtatggccgaaattaactgaaatgcagggcggattttgtgcctattgcgagtg |
| | | ccggttgaacagatgtcatattgagcatttcaggccaaggggaaagtttcctgctctgacgtttat |
| | | ctggaataacctgtttggttcttgtggcgattcaagaaaaagtggcgggtggtcacgttgcggtat |
| | | atataaggacaatggtgctggcgcctacaatgctgatgatcttataaaacctgatgaagaaaatcc |
| | | tgacgactacctgctatttctcactactggagaggttgtaccggctatcggactcacggggagagc |
| | | gcttaaaaaagcgcaggaaactatccgtgtttttaacctgaacggtgacataaagttgtttggcag |
| | | tcgcagaactgcagtgcaagcaatcatgcctaatgtcgaatatttgtatactctactcgaagagtt |
| | | tgacgaagatgactggaatgaaatgcttagagatgagctcgaaaagatagaatctgatgaatacaa |
| | | aacggccctaaaacatgcatggactttcaaccaagagttcgcataatcctaaa |
| | | (SEQ ID NO: 275) |
| 20 | 5 | gtccttaaacacgacaaaacctgtgatacttaccatggattcctctatgaaggaaaggtagtatag |
| 21 | | ccattttgggtgatacatacagtgaatgtcattgctgtagttgaagtgagtaagagcgcttaagat |
| | | taagttgagagaaaatgaaactacttgataaaaagtattacaacctcgagcccaaatatgagtacc |
| | | ttaaggactcatttattttaggactggcatggaaaaaaacagatagttttgtaagaactcacaatt |
| | | ggtatgcagatattttagagctggacaagtgtgcgtttgatattagtgatgaagtcactaattggt |
| | | caaacgagatctcaaagaacgctctttccaaaagtgatattgaattgataccggctccaaaaggag |
| | | caagctggttcattaatcaaggtaaatggactaccaataaagataatagaaagataaggcctttgg |
| | | ctaacatatctattagggatcagtcttttgctacagcagtaacaatgtgccttgctgatgctatag |
| | | aaacaagacagaaagactgttcgttgagcaatcttggctatgctgagcatgtaaagaacaaggttg |
| | | ttagttacggaaataggcttgtctgcgattgggacaatgaaagggcaagatttcgttggggaggaa |
| | | gtgaatattataggaagttctcttccgattatcgaagctttctacaaagacctatctatataggca |
| | | gggaaacagtaaataaagttagcggaattgatgatgtatatatcatcagtttagatctgaaaaatt |
| | | ttttcggttctataaaaataaaccttctgttagaaaaaatcaaaaaaatatccgctgatcattatg |
| | | cagctaaattcataaatgataatgaattttggactttggcgaatcggattttaagttgggattggc |
| | | ctgaagaatctttatctttacttgagagtttggatataaaagaaaaaaatgttggtcttccccagg |
| | | gattagcttctgctggtgctctggcgaatgcatatctcattgagtttgatgaatctttaatttcta |
| | | agcttcgtactaagatagaagacagccaaataatactgcatgattattgtcgatatgtcgatgata |
| | | ttagattagtgatttcaggagaagcactagaaagtaataagattaaggaatctattcatgcattag |
| | | ttcagggcattcttgatgagacattggctcaaaatccgtcagataatgaaccatatttaaaaatta |
| | | acgatagcaagacttatattcttgagctttcagacattgacaacggaagtgggcttacaaatcgaa |
| | | tcaatgaaattcagcatgaagtaggagcttcgagtatcccagagcgtaacggactcgataataata |
| | | tcccggcacttcaacaattattactgaccgaacaggataatttttccgaggatgttgatagtttat |
| | | ttcccgggtttaaaaatgataagtcgataaaggtagaatctgtacgtagattttctgcccataggc |
| | | tggaaaaaagtttggctaaaaaaagcaagctaatttcacctgaggagaggaaacaatttgataatg |
| | | aaacctcactgattgcaaaaaaattattaaaagcttggctaaaagatccatcaattatggttatct |
| | | tccgcaaagcgatagctatcaatcctaatctagatgcttatagcaccattcttgaaattatttttt |
| | | caagaatacaacgcaatcgtgataaacgagataaatatataatgctgtatcttctttctgatatat |
| | | ttcgtagcgtcattgatgtctatcgaaacctagaatcagaatacgtcgacgattatcaaaaattga |
| | | tgggtgaagttacattgtttgcccaaaaaatactttcctgcaaatcttttattccaaattacgcat |
| | | atcagcaagcattattttatctcgcagtgatcaataaaccatttatagctagtaataaagcttctt |
| | | ttgatcttgcaaggcttcaatgcgtcttaattaaacagcatttagaaccgttgaatagtagtgatg |
| | | gatacctatttgaggtatctgctcaaatcagtaaagactaccgagcaaatgccgcttttctacttt |
| | | ctcatacaaatagtaacaaagtagtagacttaattatcgaaaaatttgctttccgaggaggtgaat |
| | | tctggaatgcaatttggaaagaaattgttaggatgcaagataaagataggattaacgaatttagat |
| | | gggccatatcaaaatatgagtcaaagccaaatagttcggagcactatctttcatcagtgatcagtt |
| | | tcaaggaaaacccatttagatatgaacatgcgcttctcaagctaggtgtagcattagttgaactct |
| | | ttgatgatacagagaaaaacgtatggcaacctgatggtaagcagtattctccacatgaaataaaag |
| | | taaaattagaaggtaactcaacctcatggggtgaattatggcgtccaaattttagtatttcatgct |
| | | cgatagataagaaaggtgaacctggtaaagacccacgctatataagccctgagtggttggcaaatt |
| | | atccacagactcaaaatgatgaacaaaaaatctattgggtttgcagtgtgctaagaagtgctgctt |
| | | taggcaatgtagattatactcaaagaaatgatttaaaacttgataaagctaagtatgatggtatcc |
| | | attctcagttttacaagcgacgtatgggaatgttacatacaccagagtcaattgttggttcatatg |
| | | gaactataacagattggtttgcaagttttcttcagcatggattgcaatggccaggtttttcttctt |
| | | cgtatataagccaagaagatatattgtcaattactaatattattgagtttaaaaactgtttattgg |
| | | aacggctaggctacttaaataagcagatatgtatttcatcgaatgttccaaccttaccgactgttg |
| | | tcaacaggcctgaattagcatctaaccattttagaattgttacggttcagcagttatttcctaagg |
| | | atactaatttccatccttctgacgtgactttggctaatcccgatgtgcgctggaagcacagagagc |
| | | accttgcggaaatctgtaagctaacggagcaaactttaaatgcaaaacttaaaactgagtctaggg |
| | | aacatacaagcacagctgatctaatcgttttttctgagttagcagttcacccagaagatgaagata |
| | | tagttagagcactggcatttagaaccaaagccatcattttttccggctttgtcttctgtgaacaag |
| | | atggccgaatagttaacaaagctcgttggattattccagactcttcagagtctgggacccaatggc |
| | | gtgtccgtgatcaggggaaacatcatatgaccagtgatgaagtggctcttggcattcaaggatata |
| | | gaccatcccaacatattatttcaattgagggtcaccctgagggaccatttaaattaactggtgcga |
| | | tttgctacgatgcaacagatataaagcttgcggcagatctgagagatttgactgacatgtttgtca |
| | | ttgcagcatacaataaagatgtagacacatttgataatatggcttcagcactacaatggcatatgt |
| | | atcagcatattgttattacgaatacgggagaatatggaggctcaactatgcaagccccgtacaaag |
| | | agaaatatcataaattgatttctcatgctcatgggactggtcaaatagcaattagtactgctgata |
| | | tagatttagcagcattcaggcggaagctacaaatatataaaaagaccaaaacccagcctgctggat |
| | | acaatagaaaacattaaggatttttatggatactttagttaagttagctacaattatttctccatt |
| | | aattagtgctggagtagctatttgggcaattttggttgctaaaaaaaccatcagtgaaagcaaaga |
| | | aattgccaagaaaaccatcgctgatacggcctaccaagcatatttgcaattagccatggagaaccc |
| | | acaattttcgaaaggctacagcgcagattgtagacaggagcgagaccctatgtatgatcaatatgt |
| | | ttggtacgtggctaggatgatattctgctttgagaaaatcatcgaggttgaagtaaacttaaaaga |
| | | tagttcttgggcaaatacgttggaaaaacatttgaagtttcattctgaacattttaagaaaacgaa |
| | | tgttgtcgaagaggctctctatattccccctattttggatctcataagatgtgcagctaactaata |
| | | acttatcccaataggattatattccacacgataagcccactggaaaatgtaacatcccaagatagt |
| | | ttttgggattgtttcccagtgggcggaaagtatcatgatagttgtcacccccggtggagctgcaaa |
| | | gatttttatggggtgggtgttacattgcg (SEQ ID NO: 276) |
| 22 | 6 | acacgatataaaaccatctcattgcttgctgggttaactgagttgctgaatttttttctagaattt |
| | | cgcaaaatttaataggtaaaccttgtttttttaaatttacgatgatataaaaataatgccctaaac |
| | | aaaggtttaggggtattgtacaggttgtcaagcctcccacaggtcttggtgaaaccaatcactgtg |
| | | acgacggtaagcaacacttggatgatattcataattgactccacgctactgattacattatacagc |
| | | atatctaacatttgcggcgaggttcacaatttgtatttaggtactgattgtggatgagaaggttgg |
| | | agaaagaccacttggttaagccggaggatgtgtcctagaattgtcgctattctgtcatcctccggt |
| | | tttgctaatttcattcagggaatataatgaataatgatgattacccatggttcagaaaacgtggtt |
| | | atttgcatttcgatgaacctgtttcattaaaaaaagcggttaaatatgtttcctctccagaaaaaa |
| | | taataaaacattcttttctgccatttttaagctttgaagtaaaatcgtttaaaatcaaaaaagaca |
| | | aatcaacaaaacaattaagtaaaactgaaaaattaagacctattgcctattcctcacatttggata |
| | | gtcatatttatgcattttacgcagaatatcttactggacattatgaattattgatccaagaaaaca |
| | | atttacacgagaacatccttgccttcagatctttaaataaaagcaatatagaatttgccaagagag |
| | | catttgatacaattactgaaatgggtgagtgtagcgctgttgcattagatctttctggtttttttg |
| | | acaatttagatcatcaaattttgaaacaccagtggtgcaaagttattgggactgaagcgttgccgc |
| | | aagaccattttgccatatacaaaagtataacaagatattctaaagttgataaaaatagagcgtatg |
| | | agattttaggtatatcaaagaataaccccaagtataatagacgcaagatctgcacccctgttgatt |
| | | ttagaaataagattagaaaaaatggtcttattatagttaataattcccaaaaaggtataccccaag |
| | | gctcgccaattagtgctctactttcaaatatatatatgcttgactttgatattgaaatgagagatt |
| | | acgcgcaggaacgtggtggccattattatcgctattgtgatgatatgctattcattgtaccaacta |
| | | agtataataaaactctagcaggtgatgtagcccagcggattaagcatcttaaggtagaactcaata |
| | | ctaagaaaactgagattcgagattttatatacaaagacagtaccttagtggcaaatatgcctttac |
| | | agtatcttgggtttatttttgatgggagtaatatattattacgttcatcttctctcgcaagatatt |
| | | cggaacgaatgaaaagaggtgtccgcttagcaaaagctacaatggacagcaagaataggattagag |
| | | aaaataaaggtgaagctttaaaagctttatttaagaaaaaattatatgccagatattcacatattg |
| | | gaagaaggaattttttgacttatggttatcgcgccgcgaagatcatgaattcgaaagctataaaaa |
| | | gacagttaaaaccattgcagaaaagattggaaaatgaaatactaaaataaatatttgctggcccga |
| | | atcatacagggccacaatacagttgaaaacaagctataataaacaacatctaatttttatatac |
| | | (SEQ ID NO: 277) |
| 23 | 7 | tctcaacttccccaaatgtccgtattcatccataaataccctgatttataacaattttaccgtttt |
| 24 | | ttagtccatcatcgtccgcagccatccagtagaatccgataaagaatgtgtataggattgtgtata |
| | | tgttcctgttcggtcatggattcctatacacatgcctttaaacgatatgcagattcgccgcgctaa |
| | | gcctgaagataaaccctatacgcttggggatgggcaaggcttgtcattgcttatagaacctaatgg |
| | | aagcaagagctggcggttccgctatcgctatgccggtaaacccaagatgatctcgcttggcgttta |
| | | cccaacgatcactcttgccgatgctcgttcccgtcgtgatgaagctcgaaaacttgtggcagaagg |
| | | aaagaaccctagtgatgttcgaaaagagcaaaagctggctctgcaagcagagtcagagaacgcctt |
| | | cgaaaagatagccagagagtggcatcaacttaaatctgctaaatggtcggcaggatatgcatcaga |
| | | catcatggaagcgtttaagaacgacatttttccttatgtgggaacaaggcctgtgagtgagattaa |
| | | accgctagagctgctgaacgtactgcgtaaaattgagaaacgtggtgcgttggagaaaatgcggaa |
| | | agtgcgtcagcgttgctctgaagtgtttcgctacgcaattgcaacgggtagagcggagtacaatcc |
| | | tgcggcagatctttccagcgctctcgaagtgcaccaatccaatcatttcccgttcctaaaagctga |
| | | tgagatacccgaatttctgcgtgccttagagagttacaccgggagtaagcttgtccagatagcaac |
| | | gaaattactgatgattacgggcgtgagaaccatcgaattacgcgcggcattatggcaagaatttga |
| | | tctggataacgctatttgggaaattcctgctgaaaggatgaaaatgcgcaggccgcatcttgtgcc |
| | | attgtcgacccaagcgttagatttactccatgaactcaagataatgacagggaactatcgttatgt |
| | | ttttccaggacggaacgatccgaacaaaccgatgagcgaagctagcataaatcaagttatcaagcg |
| | | tatcggttacgaaggccgactcactggtcacgggttcagacatatgttatcaacaattttgcatga |
| | | agaaggttttcaatcagcatttattgaagtccaattagctcatgttgatagaaataatataagagg |
| | | aacttataatcatgccatataccttatggaaaggcagaagatgatgcaatggtacagtgattatct |
| | | tcgcaaaaaaaaggggttataatatgttaaaccagtcattttccgtttcgaacttaattaagcttt |
| | | taaaaaaaaccgatccaaaaagatacaaaattggtaggaattcagctgaatataaaaaatatatag |
| | | ctgataaagttaatggctcaattgaaacatactcatttggttcgatctcaaattcaagaattaaca |
| | | acaaaaatgtgtatatatttaaagattttatggatgtacttgtcgccaggaaaataaatgataaca |
| | | ttaagcgtgtgtatagtgttaaacaaaacaacagacatgacatcataaaaaaagtaaatacagtgt |
| | | taagtgagcctgtaaattattatatttacaggctggatattaagagtttttatgaatcaatagata |
| | | aaaatatcgttttccaaagaattaataataacccgattatttctcataatactaaaaaatttatca |
| | | atggtctttttaaacataacgctttctctgcaaataacggacttccccgtggtatgggattaagtg |
| | | cgactttatcagaaatatttatggaggaatttgatgctgagttggcgaggctgcctgaagtatttt |
| | | atgcttcaagatatgtggatgatatcatagttttttcattctataaaataccagattataaaaatt |
| | | atttttcaaggattttaccaaatggattacatttaaatgaaagaaagtgcagtgagtataccatag |
| | | aggacacttcaactaaacattctgaaattgagtttttgggatattcatttattatacaccatggat |
| | | taaaaaatcagcgtcgtcatgttgtgatcagaatttcggaggagaaaataaagaaaataaaaagaa |
| | | ggattgcacttgcggtaaaagattactcaaataattctgatgcagaactcttgaagaaaagaataa |
| | | agtatttaactggtaatatattagtaaactccaatagtaataaaactgatgctttatatagtggaa |
| | | tttattacaattatcaacatttaactgataaaacacagctcaaggaacttgatatatttaagaata |
| | | ggatgctattttcttcaaagggcgaggtggggagaaaaattttagcagcaggtcacaacttattaa |
| | | ctgcgcctaaaaaatactcatttttggctggttttgaaaaacggctactgtcttcttttaaacggg |
| | | aagatattattaaaataaataaggtttggtgattcatgaaaattaaaatatcgaagagtgattata |
| | | aaagagtacttctcacggatattttaccatatgaagtccctatccttttttctaacgaaggtttct |
| | | ataagttaatttctgaaaataaagttttacccggaacattttcagaaggccttaagctggattctt |
| | | ataccatcccttactcctataaaataaaaaaggggctggcgagttctcgaagccttggcattatac |
| | | atccttcaacgcagttaagaatctgtgatttttatgataagtatgaacatttgatggttcatatgt |
| | | gtacaaaaagtccgttttcgctacgttatcctagcaaaatagggagctattattacgaaaaggact |
| | | tcttaaaaagtagaataaatctaaaagatggtcttgtacaatttcataatcatggctttgattccc |
| | | aagaaacttcctcatcttcccatttttcatataagaaatatcctttcatctataagttttatgagt |
| | | catatgaatttcatagattggaaaggaagtttaggaaacttttaaagcttgatattgctaagtgtt |
| | | ttagtcatatatatacacacagcgtttcatgggctgtaaaatctaaagaattctctaaggttaata |
| | | gaacttataacagctttgaaggttgtttggataagctttttcaagatgccaattatggtgaaacaa |
| | | atggcataataattgggcctgaattttcaaggatatttgcggagattatattacagcgcgttgact |
| | | tgaatgttgagtctcatttgaatcttgagccaggcatagttaaagataagagctatgctataagac |
| | | gttacgttgatgattattttatatttgcggatgatgatgaaacatttaagctaatagaatttgtac |
| | | tggcaaatgaactcgaaaaatataagctttatttgaatgaatctaaaaaggaatttatcgagaggc |
| | | cattcgtgactggagctacgatggctaaaaatgatattgcagaaatcattgaggatttatatggat |
| | | cgttaatccatactgagaagttggatgagttaacagctatggttaatttaaatccagacgtcaaaa |
| | | ttcagcctgaaaatatgaatgacctttttccattgaaaggtgtgtggaataaaaagctacacgcgg |
| | | acaaatttataaaacgaatcaaaattgcggttagaaaaaacaataccacatttgatcttgttagct |
| | | catacttattaagtgcgattaagagtaagtttttcaaagtaattaggctgttgaggatgttcgatc |
| | | tgtcaggaaaagaagatataacttataaattcttctcaatattcaatgaggtgattttttttattt |
| | | atgctatggattttcgagtccgacagacatacataattagccaagttattttggaaataaattcat |
| | | ttgctaataagcaagcttcagacattagtgaagttataaaaaagaatacttttgatgagcttctta |
| | | tgtgcatgaaaagcatgggtaatattcatgagaggccagtggagttatctaacttacttatatgta |
| | | tgaaaggtttgggggagcagtataaactcaatccagatgaatttaaggatttgttgggtattagtg |
| | | agaatgagtgtttttacgatttagaatatttttctatatgcagcatgttacactatataggcgatg |
| | | atgttctctatctaaaaatgaaagaagatattgtccttgctatacagagtttgataagtggtcgga |
| | | acgatataaaaaaagacactgaaacatttatgctattccttgatatgatgacgtgcccatatctta |
| | | cagttaagcataagagaataatttatagaacatatgtcgaagcaaatacaggtcaaaaaagattta |
| | | cgaatgcagtaattgattctgaaattgattctttaaaaaataatgtaatcttttttaactggtctg |
| | | gagatgctgatcttgagcacgttctttataaaaaagagttgcgaacagcatatgaatagtagtatt |
| | | ttaatttcgttaaagggttgcgatgcctaaggtttcgacctgaagcagataccggaagatcggctt |
| | | ttgaatgttcatccgaaagatattcgcgatacgttttgaggatggaccgatttagacacactattg |
| | | ccttttagctaaacaggccgcgaaagcggcctttttaatgaatcagatttcccctcaccgatctca |
| | | atacttcccctcagcgtgcgcagccccgcccgcctgcccgcttcgcttaacagactggttttcatg |
| | | caccccttaaatcgtctcagaagccaccacacaagggctttcgcgtcaaaaatggcgcatgagact |
| | | catgcgttttcatgcgccatagatatgcactcatacgctctcaggccagctagggaaaaagcgtaa |
| | | aaaatcccggtactggaccgagacttcgtgggcgtattttgctaa (SEQ ID NO: 278) |
| 25 | 8 | agcatcggagcaaagtaactcaataccgaacaataaatatgagcccttcgtgaaaccgggtaaggt |
| | | caaactcataaaccaacaaaaggggaaaagtgggatatgtgaggcgtgtatgatttttatttattg |
| | | ggcttcgttaaaaatggtgatttaatagccctttaaatttatcactttttaactaactccgagggt |
| | | ttatggtcatttttgacgagaagcgacacctgtacgaggcactgctgcggcataactacttcccta |
| | | atcagaaaggctctatttccgaaatccccccttgtttcagctcccggacctttacaccagagatcg |
| | | ccgagctgatctctagcgatacctccggccggagatctctgcagggctacgactgcgtggagtact |
| | | atgccaccaggtataacaatttcccacgcacactgagcatcatccaccccaaggcctactccaagc |
| | | tggccaagcacatccacgacaattgggaggagatcaggtttatcaaggagaacgagaacagcatga |
| | | tcaagcccgatatgcacgccgacggcaggatcatcatcatgaattacgaggatgccgagaccaaga |
| | | caatcagggagctgaacgacggattcggcaggcgctttaaggtgaacgccgatatcagcggctgtt |
| | | tcaccaatatctattctcacagcatcccttgggccgtgatcggcgtgaacaatgccaagatcgccc |
| | | tgaacacaaaggtgaagaatcaggacaagcactggtctgataagctggactactttcagcggcagg |
| | | ccaagagaaacgagacccacggagtgcctatcggaccagccacatcctctatcgtgtgcgagatca |
| | | tcctgagcgccgtggataagaggctgcgcgacgatggcttcctgtttcggagatacatcgacgatt |
| | | acacctgctattgtaagacacacgacgatgccaaggagttcctgcacctgctgggcatggagctga |
| | | gcaagtataagctgtccctgaacctgcacaagaccaagatcacaaatctgcctggcaccctgaacg |
| | | acaattgggtgtctctgctgaacgtgaatagcccaaccaagaagcggttcacagatcaggacctga |
| | | acaagctgagctcctctgaagtgatcaacttcctggattacgccgtgcagctgaacacacaagtgg |
| | | gcggcggctccatcctgaagtacgccatcagcctggtcatcaacaatctggatgagtataccatca |
| | | cacaggtgtacgactatctgctgaatctgtcctggcactaccccatgctgatcccttatctgggcg |
| | | tgctgatcgagcacgtgtacctggacgatggcgacgagtataagaacaagttcaatgagatcctgt |
| | | ctatgtgcgccgagaacaagtgcagcgatggcatggcctggaccctgtacttctgtatcaagaaca |
| | | atatcgacatcgacgatgacgtgatcgagaagatcatctgctttggcgattgtctgtccctgtgcc |
| | | tgctggatagctccgacatctatgaggagaagatcaacaatttcgtgtctgatatcatcaagctgg |
| | | actacgagtatgatatcgaccggtactggctgctgttttatcagagattctttaaggacaaggccc |
| | | caagcccctacaacgataagtgtttcgacatcatgaagggctatggcgtggacttcatgcctgacg |
| | | agaattacaagacaaaggccgagtcctattgccacgtggtgaacaacccctttctggaagacggag |
| | | acgagattgtgagtttcaacgactacatggctatcgcatgacttttaggcctcatt |
| | | (SEQ ID NO: 279) |
| 26 | 9 | aagtgaacggatgtatattgagtgcaatgtgattaactatctgttgttacaatatttagataggtg |
| | | ataaaatatgacatctaccattgatttttatgaatctgatttctcagccacattatacccattaaa |
| | | aaccaatcaaatattactcaagcatcactcacaagagatgtcagaatatatttatcagaaggtcat |
| | | taatcctgcatatccaacagatagttttctgtctcagcaaaaagtcttttcgactaaacctaaagg |
| | | tcatttgagacgaactgtaaaattagatccagtagctgagtattttatttatgatgttatctatcg |
| | | aaacaggaagatatttaggccagaagtaagcgagtcgagaaaaagctttggatatatttttaggaa |
| | | cggtagcaggatacctatccacgtttcctataatgaatataaacaaagcttaaaaaaatattctga |
| | | gctatattctcacagtatacattttgacatagcatcttattttaatagtttatatcaccatgatat |
| | | aatccactggtttagctcaaaagaaggagttagccctgcggatgttgaagctctcggacagttttt |
| | | tcgcgaaattaactcaggacgaagtatcgattttatgccccaaggaatttatccggcaaaaatgat |
| | | cggtaatgagtttctaaaattcgttgatttacatggtcgcctaaaatctgctcaaatagtaagatt |
| | | tatggatgactttactatttttgacaatgacattgaaacactaaataatgatttcatcagaataca |
| | | gcagttattagggcaagtatccttaaatataaatccgtcaaaaaccacatttgacaatgtgatggg |
| | | agatgtgaatgaaaccttaactcagatcaagtcatcacttaaagaaatcattacggaatatgaaca |
| | | tatacctacagcctcaggggtagaggtagtcgagactaatattgaaatcataaagcaccttgatga |
| | | tgaacaagttaacaaattaatagacttgctaaaagatgaaaaaatagaagagtctgatgccgattt |
| | | aattcttggttttttgagaactcataatgatagtttactttctcagatgccaatgctattaggcag |
| | | attcccaaatttaataaaacatatttatacgatctgttcaggtattaccgataaatcaggattagt |
| | | aaaaatattgctcagctatttaaatactaataataactttttagaatatcaattgttttggattgg |
| | | agcaatagttgaagactatctattaggtgtaggtgagtatggctccgttttacacaagttatatga |
| | | gttatctggtgattttaaaattgccagagcaaaagtattagagataccggaacagggttttggttt |
| | | caaagaaataaggaatgaataccttagaaccggacaatcagattggttatcatggtcttcggctat |
| | | cggtacgagaaatcttaaatcagcagagagaaactatattcttgattatttctcaaaaggctcacc |
| | | aataaattatcttgttgcatcttgcgtcaagaaactttaatttaaaagccaccttcttgaaaggtg |
| | | gctttaaaaaatacctttagttcc (SEQ ID NO: 280) |
| 27 | 10.A | gaggatttatgcacaaaatcctgatgcgaaatgttttcaaaaattgtcaggttaacgttcctgcag |
| 28 | | atctttgcgttacatgtcatttctggatcctttcccgacaggttaggttgtgattgatatgatgcc |
| | | catctctcattttagtgatcgttatccctttataaacaggagtttatatgttatctatatgcaata |
| | | gacttaaatcgatatacgtgcgcagcttacgattcacctctctacttactatttaaggaaaagagt |
| | | gaggggagaattgattttcattaagatattatgagagaattatgactagtgaaatagtgttaaatc |
| | | ttgatttcccagaatataaggatgatttttgtactgatagcattgatgagcaagataatgagttgt |
| | | ggcagcaacaggccaataaaaagctactttcgtttctcgaggtgatgggggaggaagcaagacgat |
| | | ataaagaaaataattcccgtagtacgcatccacattataagacattgagtagttatcaccatgcaa |
| | | tctttatcagtggcgcgcggggggcggggaaaactgttttcatgagaaatgccagatttagctggc |
| | | aaaaacattataataaagatctaaaacgccctaagctatattttattgatgtgattgacccgacgc |
| | | tattgaatattgatgaccgtttttctgaagtcattatcgcttcaatatatgctacggtagaaaagc |
| | | ggatgaagcaacctgatattgcgcagaatatcaaagataattttattaattcgcttaagacgttgt |
| | | ccggtgcattaggtaaatcaaaagattatgatgaatataggggcattgatcgtattcaaaaatatc |
| | | gttctggaatccaccttgaaaaatatttccatcagttcttgatttcaagcgttgagttactggatt |
| | | gcgatgcgctggttttgccgattgatgatgttgatatgaaaatagataacgcttttggtgttctgg |
| | | acgatattcgctgcctgttgtcatgtccattagttctaccattagttagtggggataatgatcttt |
| | | atcggttcattgccaaaagtaaatttgaggaattattaaatcgtaaagcaaactctaattatgcta |
| | | aagaaggcagcgagatagcagaaagattatcagaagcatatattactaaagtattccccagccatg |
| | | tgaagatacccctccaaccgatagatgagttgttgccatatctttatatacattctaatgaagatg |
| | | aaaataaacaacatacaagctattctgaatttatcaaacttgtacaacaaaaattctactttcttt |
| | | gtaatgggcaagaacgaagcacaaattggccgcagccgagaagcgcacgtgaagttacgcaactaa |
| | | tccgttctttacctccgtctactcttagtaaggaagatgattcgggaactgatttatggcaacgct |
| | | tcgctgtctgggcggaagaacgtcgcgatggattagcattaaccaatgttgaatcttatctgttta |
| | | ttaagaatgcgaaagcagtagaagatttaaatctgtcaaatcttattgcttttaatcctttactgc |
| | | aaaaaggaaaatatccctgggcagaaaaggatttttataaacagcagtcccaacgtcggaaagagc |
| | | tcaatgcccccgaaacaaattcaggtatccttaataccgtattttccgaacaaaggaaagatttta |
| | | ttttaagaagtatgcctgcgctggaactcattatggagcctatgtatgtcactaagacggtagcag |
| | | aaaaaaatgataattctgcgcttatagcgatctatacccattctgattattacagccagcagcaga |
| | | acagacgatgtcatatattttttggcagagcttttgaaataatgttctggtcagtattagcgaaaa |
| | | ctgaaaatcttccacaagaattttatgaaaaagataagtttaaatctttatttggtaatattttca |
| | | aaaaagtaccattctactcaatattttcaatgaaccctacaaaggttgttgatgaagaaaatgacg |
| | | atggcagtgaacctgatttttcgcaaaaactggacgatagcattaatgaactggtggaagatatat |
| | | atatctgggcaaccagtaataaattgcgagccttcaaaaataaaaatttaatacccttaatgacgt |
| | | gcgtttttaataaggtattttcacagatcaatgtactgagaaaaaacgtgcaggacagagttaaat |
| | | ttagagatgaacatttgtcagatctggctaagcgatttgagtatatgtttattaatgctatcttta |
| | | ctttcatcagagaaggggtagttgtcaataccaatgtggcaacaggcgcagctcctgccagagtac |
| | | gtaatttatcagagtttaataggtatgataaaacattatccaggaatatgtccgggattttatccg |
| | | tgaaagaggataatggcttaacgatagtcaaagagagtgagggcgatatcgcagatctgttatttg |
| | | aaatttggcatagcccattatttaaattaacaaccaggacatgttacccaataggtaaaataaatt |
| | | cgcaaaatacggcccaggaaaatttatcatcagattttaattcattttttgaaaatggtatcaact |
| | | tcgaattgataaaacaatattattggcaaacttcaaatcatgataatatcaggacagcagacgtta |
| | | gggaatgggcaacttcacgtcttaatgaagcaatcatccttttttcatggatgaaagaaagcaagt |
| | | ctattaaagcgaaaattgacggacagagctacgagggtcggctctttcgcgggcttcagcaggcgc |
| | | tggaaggttatgaggaggtctgagtatgtttaatcaggatccttattggctcattcctaccctttg |
| | | tctggcatcagaccgaattttttatgcacaattgcgagaccacttaggccagaaaagtagcggtga |
| | | acgcaaaaaagaaaaaaatggatatatactggtacaggcggcacaagactatcaattctattttgg |
| | | cggccgtattcggaaagaggatgtgcaaaataatgccttaatgtggcagatagaaactggtaatga |
| | | aaattgcttatcgatgcttgatagtttgtcagcatatttcctcacatggcgcggcaattgttttga |
| | | ggtcaggcgtgagcgacttgaaccctggctgatgatctgttccgtgatagatcccgcatggattat |
| | | tgcctatgcataccaacaattgattaaacaaaatgttgtatgtgatagtgagcttatttctttgct |
| | | gacagaacatcaatgtccatttgcctttccaaaaggcagaggggacatttcctttgctgataatca |
| | | tgtccatcttaatggtcatggttatagttcaatttcaatgctgaactttatagatggaaattataa |
| | | ggttaaaaaagggataaaatggccctatcggcaggaatacaccctctttgaaagtggtcttctgga |
| | | taaaaatgatcttccccgctggctgtccgcttatagctcttgcttacttaaaaatgtatataattc |
| | | atttcaacaaggaaaaagatccgaggtagatttcacatgtctgaaggatgcggtcgaaacggtgct |
| | | tgcggatgaggataaatattattttttagaggtagcttcgctatatgatgttgtcaccttgcagca |
| | | aagagtgctttatgaagccgcccagcagaaatatcactcacatcaacgttggttactgtatacttg |
| | | cggaataatgttaggtacagaatctgaagattatgcgaatgcgctggctaacctgatccgaatcag |
| | | caatattctaagaaactatatggttgtatctgcggttggattgggacaatttattgattttttcgg |
| | | cttcaactatcgtcgaataacaaagccagctgatacaaacaaccgagttcattatgattcttctgc |
| | | tggtatttccagagaatatcgtgtctctcctgattttgtactgggtagcggcgtaatgcctgatat |
| | | atatgccaggcaacttttcgatttttattgtacccaagcacgcaagggcgtacccgaacaaggaca |
| | | tattgttgttcattttacacgttcctttcctgacaaaaaatcaacatatgataaattgctaaccga |
| | | gtgtcgcgaacggttacgttctcagtgtgattattttggccgttttttaacatcgcttactttgca |
| | | gtcgatagaatataaaaatttatctactgatgaagatcgaagcatagacattagaaaattagttcg |
| | | tggctatgatgttgctggaaatgaaaacgagctacaaatagaggtatttgccccggttctccgggt |
| | | actgcgtgctgctaaatttaaaggggagggggtgaactttaaaaggctacagcgcccttttattac |
| | | tgtacatgctggtgaggattattgtcatatactcagtggccttcgggctatggatgaagccgttga |
| | | attttgtatgttaggagaaggcgatcgtatagggcatggattagctctgggagtagatataaaact |
| | | atgggcgaatcgccaaaagcgagcatacctgacggttggacaacatcttgataatttggtttgggc |
| | | atatcatcaggcagtattactttctcaacatattgtcgagcatataccagtaatgcatgaattaag |
| | | ggataagatccattattggtctcatcaattatatagtgaaacttatacgccagatttactctttaa |
| | | agcatggctgctccgccgtaactggccggattataagtcaatcatatctgatccagcaaatatcaa |
| | | tgaatgggtgcctgaccaacatattttagtcagtacagatgagactacagctaaggccagaaaaat |
| | | ttgggaacgttatttaaatagcggtctggcagaaaatgatgtttttaacagaataatttcagtaaa |
| | | ttgtgcgcccgatacagcgcaaaatttttcaatgacctttaatgaaaatgaagatattttatccaa |
| | | aggggaattattattgtatgaagctatccaggatttcttaatcgaaaaatatagtaggttgggttt |
| | | agtcatagaagcttgtccaacctcaaatatttatattggcagactggagaaatatcatgagcaccc |
| | | attattccgttggaatcctcctgactcccaatggattaaacctggtgggaaatttaatcgctttgg |
| | | attgcgcacaggacctttatctgtctgtataaatacagatgacagtgcattgatgccaaccacaat |
| | | tgaaaacgaacatcgcttaatgagagactgcgccatacatttttatggtattggaacatggatggc |
| | | ggatttatggataaactcaatacgcataaaaggtattgaaatattcaaaggtaatcatttaagtca |
| | | ggatttagataatttaatctaaatgtaaacaagaaatccacgcaaatgcgtggattttaagtcaac |
| | | ttattattctctgaaacggtttaaccgttcggaacaacagattaaatc (SEQ ID NO: 281) |
| 29 | 10.B | tgtggttagttatcacagcactaacctattttcgagctttttgattgaccaataccatttctttta |
| 30 | | attatgaataatgatgcgtcaaccgatggcgaacgggccaaatccactcttctacaactgcccatt |
| 31 | | gtcacggtgtggaataattaaaaattttagatttttgagattattctcattaccatcttgatttta |
| | | tttggttttgcatcaaaattcatagttcacaagcttttctcactccaaaaacaactgtaaagggat |
| | | tattgtgaacacgatatacataccattagacagcggagagtctgcggttcttaaggatccagatac |
| | | cttacttccccgaaatatttacgaacagcttactcgatttattgaaaaggctgttaatgaagtacc |
| | | gaagcctcacgaagcgcttaatgaaacccgtagccataaggctatatcgattgacggcgcaagggg |
| | | gacaggaaaaacgtcggtgctagtgaatttgaacgactatctgcagagtaatgctcagcaactggc |
| | | ggggaaaattcatatccttgatcctatcgatccgactctacttgaagatggtgagtcgctgttctt |
| | | gcatattattgttgctgccgtgcttcatgataaagagatcaaaactgcccaaagcagagacctcga |
| | | taagtccagagtgtttacccagaagcttgagaacttggcacacggactggagtccgttgatttgca |
| | | acagaatcaacgtggaatggataaaattcgctccttatatggcagcaagcatctggcaaattgcgt |
| | | tgaagagtttttaaaatctgcgttggagttgatcggaaagaaattattgatactaccgattgatga |
| | | tgtggacacttcactaaaccgggcatttgaaaatctggaaatattgcgtcgttatcttacctctcc |
| | | gtatgttttgccggtagtgagcggcgatcgccgtttatatgatgaggtctgctggcgagattttca |
| | | tggaaggttgaataaggattcagcatataatcgcaagaacacatatgatattgctagagatttggc |
| | | aattgagtatcagcgtaaaattctgccgctaccgcgcagactgagtatgcccgatgtaagtgatta |
| | | ctggcagcaagatggtatcgaagttacgctagataaaaatggcattcctctgcgtaattttatggc |
| | | atggttgaaaatatttattactggccccgtgaatggccttgagggtagtgatttacctctaccgat |
| | | accttcaatacgtgctttaacccagttcatcaaccattgcagggatttaattcgtgagcttcctga |
| | | accattcagaaagaaagtcagtacgctggccttacgtcgtatgtggcaaatgcctgatgttcctct |
| | | tgatgttcttgaaagttttgctgaaaaacatcgggaattgagtaaagaagctaagcgtgaatatgg |
| | | ggaggcttacaagctattttatgatggactaaagaattttactgcttgggatagtaaggcttatct |
| | | agaagatgataaacaatctgcatggctcgataggttgtgtgagtattttcgttttgaacctaaggc |
| | | tggggctgtgtttttaacgcttcaggcaaaacagttctgggtctcatgggcgcagggtgacaatcg |
| | | taatcaatcgattcttgcgactccgctttttcaacccttattgcataattttcgtgaatacgatgt |
| | | ctttgaaaggtatgatgatctttctgattgggaatctcagttaagaacaaggttaccggagagttg |
| | | gttgactgccattaaagggcaaaaaacgcttttaccctatcctgtagcagaagcgggaattaatac |
| | | cagtttaaagtggaggtattgggaagaattagagaactatgggtttgatcctgctttggaaagcaa |
| | | ggcaaatttccttttgtccacgttgatgcagaggaatttttatacaaactctaaacagtcagtcgt |
| | | gataaatattggtagagtttttgaaataattattgctagtcttgtttcggatttagagttggccga |
| | | cttgcagagaattagacaacgttctccattttactctgctagcgcgcttgcacctaccaaaacgtt |
| | | agatttggaagaggattttacgaaaaagaatacaagatttatgaataacagaagtgaaactgacag |
| | | agacatttctgatgatattcttgttgatgtgccggataaaaatgaggacgcatggaaaaaaatttg |
| | | tgatgaaataaaccattggagaaagacacacaatgtggctagtacaaacttatcaccttggctggt |
| | | ttataaggtctttaataaaacatatagtcaggttgctaataatgtgtttgttcccagtggaatgca |
| | | aaatgttgatgcggctctaaatgtttttggtagggttttttatgcagtttggtcagcatttggtag |
| | | ttttgaaaaaggcgaattgttcggactatccgatgtggttgctacaactaatattatttcggcaaa |
| | | aaatttttataatcatgataacttccgagtgaatgttggaccgtttacgcctgagcaaaaccaaaa |
| | | ttctgacagcgatcgtgaggcatatcagcatcgcaaaatgtatggtgaaaaaaccagagcggtaag |
| | | ttatgtattagcaactcatccgctgaaaaaatggatcgacgaggtattacgcactgagtttaaaca |
| | | aaaacagaatgctcagattcagaccgagagaaaaatgccgattcaggctgagaaaattatagatat |
| | | cagcccggcaagagagtttatcacaagaaaactttcattaaattcacactcccggttggttaaaac |
| | | acgtataataaaacagcttaagatgttatatccaaactacgataaggctaaggacttcattgatga |
| | | agttacaaaccacttccctcagaatgatcccgcaattaatacgcttcagaaagcatttgcagaact |
| | | ttaccccgatggtgacaaataatgttaactcggtctctaagtgaacatgctgcagggtgttttttc |
| | | actgatgagcgtctgtcacaacgctttctagatatccttttatcgccacccaaggattttgaaacg |
| | | tggtcatcattgcaggaggaatctttcaagctgctcgttaagagcatcgatagccgatatccacgc |
| | | acttaccggttaaccgacgtacgccagcttgtggggaacatatgtgacaacgggttactgacgagt |
| | | ccgacactaccttggctcgatgtcattgcggatcagttactgttgcggaatggcgacttactctat |
| | | taccgcgaaaataaggttcaagactacgtgcgaatagctgcggaactcgaccctgcccttctagtg |
| | | ggatggcgtcttggcgactggcttttgcaaagcccaccgccgcgattgacggacataacccgtgtg |
| | | gtgatggcgcagaatccgttttttgctccacctgctaatgcaggtaaaccttttgccgaggggcac |
| | | gtacatctcgggggagtgacggctggagatactattttggatggctatctttttgaagagattgaa |
| | | ctacccaaaagcaaagatatgttgttgtgggcgcacaaagagcatgatgagttaacaccgttgata |
| | | aatcgagcaaagtctttgcttacagttctactttctgccccccctcaaacggtttctgagcaaact |
| | | caaaatggttttgatcagcgtaaaactgtatctgagaagtacaaggcattacagaacccaatggat |
| | | agcatccatcgtctcccagactggttattgcttgctaaaaagaatcgcggaactgaaagcgtcagc |
| | | cccggctggtttttaaaccaactggcgcatgcctccgaaaaaaaacatccctcgcgctggctgtgg |
| | | ctgcagctatacctttgccactcttatcagcttaaagacactcatccactggagcgcacggcaata |
| | | ctctgtttttggcttacggtaaatgcgctacggcgtcacattattatggacggacaggggcttgcg |
| | | tgttttaccgagcgttattttaatggtgctttacgtgcgggtaagaaagctgacagtagcaatatg |
| | | cgctacctgtttgccggtaaagacgatgtggccgaagtgaaagcatccccaaaggctttcgatcat |
| | | gagatggtcactggattttcctcgacattgctgaaaaccctcggcattccagctgtttttccaccg |
| | | tatatttttggtgagcatgagattaagccagatgaacgcgtgctgcgctatattggagcactggag |
| | | cgctggcagttttgtgggcacttttctcgctctaaaactgcaagtcgcggcaagcgagcaaaggct |
| | | gatttgcaggctaactggacagaagcggagcgattgttacagaaactgtacagtcataatggctgg |
| | | aatcatcccgtcttcttagggggtaaacgtaacccacattttcattttcagccgtcgaactggttt |
| | | cgggggcttgatgttgcaggggatgaaaacgtactaaaaattgcaggctttgccccgatgctgcgc |
| | | tggctacgaagtggattatatcccgtaccagaagggcttcgcgccagtatgagttttcatttcagt |
| | | attcatgccggggaggattacgcacatccggcgtcaggattgcgtcatattgatgaaacggttcgc |
| | | ttctgcgaaatgcgggagggagaccggctaggacatgctctggctctcggaattgaacctgcgctc |
| | | tgggcgaaacggcatggtgaaatgatactacctctggatgaacatttagataatcttgtctggcag |
| | | tggcactatgctacgcttttatcggcttcattgcctctcgctcaggcggtattaccgctgcttgag |
| | | cgtagaattgcacgctttattgcacggtgcgaatggtgcaaaaagagacctccgcaaatagataac |
| | | agtgtggtggggaaacaggcctgtagtgatgataaacctctggaaaatattacacctgatacgctc |
| | | taccgggcctggctactgcggcgtaattgttcatatcgactccagcaactccacggcggttcccct |
| | | ttgacctcgcaagagaaatgtgcgctgccggattgggccacgctcagcgataaaggcaatgtggcg |
| | | gcgcagctttatcagcaaagacactcgagtctccttgacgatatgccgccgcaactggtagttgtg |
| | | cgtgtagcggacgaatggggaactcaggagcttattggcttgggaaatcctggtaaactgcgtcag |
| | | caggctcttgacggtaaagatatcctccaagacattgatacgccggtagagctgcaatttatgcat |
| | | gctttacaggactatttgctagatcactatgatcgtaaagggttaattatagaaaccaacccaaca |
| | | tcaaacgtatatatcgcgcgattcaaaaagcacgtagagcatcctatttttcgttggaatcctccg |
| | | gatgaagaactgttgaaaccaggcgctgaatttaatcgttatggattgcgccgtgggccagtcagg |
| | | gttctggtcaatactgacgatccagggattatgcctacgacattacggacggaatttttactactg |
| | | cgagaggctgcgattgagcgtggtgtcagccgaacgatggcagaatattggctggaaaggctgcgc |
| | | ctgtacgggctggaacagtttcagcgtaatcatttaaatgtatttgaagttattgaatagaggatt |
| | | ttatcgtgagtggtacattcccttacttgcaatatacggatgtcaatgggctacaacctaagctca |
| | | aagaagagttgaaaaatttacggagaaaagagtatttgtcctactggcctcgttttctgatacgta |
| | | gaatttcgctttatgctcttccattcctcatgttcttcacttttttcttttgtctgagtctgacga |
| | | agaaagttggggcagaggaagtgactaatattcttggaaccgtgagtatatccttcagtagttgcc |
| | | tgctgctggggattattatttctggtgtcgtgttactcttgcagtggacgtgcttcaactgtaaat |
| | | acagtccgcaggatacgaatggagttgttggggctcgtaagttaaattataaattacttgctcatg |
| | | ttgtatttgttattgcatgcgtgcttttatttgtttttatttattgcaccaataataaagtgtttt |
| | | atggttttatcgtgtttcttggtttgacattattaccattggtaattgaccgtaccttgggggtga |
| | | ctcgtcaaaatgaacgtcacaaactctatatcagaaggttagagcgcctcgatgaattgaatattc |
| | | tccgggagaaaatgaatattaaattcgaagaatcccatttcatcgagtatatgaagcttgttgatg |
| | | aagctgatcacggaaaaaaccaggatacagtaagcgatacatcctattttatgacgttgatagaaa |
| | | ataagctaaaagtgtaatcggttttaatatgatgctgtataaaaaactacgcaattgcgtggtttt |
| | | ttgtcggactatgagggcaaggttgccctaaaacagaggttaaacgttgggatgtgatttattgca |
| | | catcatgccgtgcccatccagtagaatccggttcgaaatgtgtataggattgtgtatatgtttctg |
| | | ttcggtctcggattcttatacac (SEQ ID NO: 282) |
| 32 | 11 | ttttagaaatattgtgtaaaacttcttactctttactggtcatccctcagtcgtggaaaaaacaca |
| | | ctgttccatataggttttatttgtgatataatgaacaagttcttatttaagaaacctataaacatt |
| | | aagcgacggaaatatatcatgaaaatagtcagcaataccgtttgggatggacttaaactgcctgat |
| | | tatagggctcgtttttttatagaagtttggaaggagattttgtacgtcaacactccttcattttat |
| | | caatctaaaatgattaatacgatgtcaggtgccgaggagttagtcgaagccattgatgattacata |
| | | caagatgataagagtaaaaaaagcttattatcaatgatagaagattacaaaggtaatttaaaaaaa |
| | | gactctatagcaaaagacacttttaaaaacttgcatgcaacgctgttaaaaaaaattgagactgtt |
| | | cctgacccaatatctagtaattatattttagaattaaaaacaattgttaaattagtattatccaaa |
| | | gaaagtgactattatcacgaacttaaaaagcagctaaaatcatctattttgtctaacgctgatttg |
| | | aataaaaaagcccgtttaatggactccatttatcaattaactaaaagctttattggctatctcctg |
| | | tggaaggggtattcaccaacttatttatataatagaatggagtatcttacgagaattaaaaattat |
| | | ggcagtagagacttttccgctcaatttaatagttgccttgataaattaactattaggattcatgat |
| | | tatacagtttattttcttattacccctttgtctaaatatctgattgaattgaataatatccttgat |
| | | gttagctttatcaatcgagaaggtattattaatgaaaaaaactacaataaaatttcacaaggggtt |
| | | gaatcttcggtattagccaaaattgttgttaatacaacagactacgtttccgcggcgtggcaggca |
| | | aatgaaaaactggataaagtcatagattatttagaaatagagaagccagaatataatattagatat |
| | | tctcctgtatgtcttacagagttttcaaatggtagattcacacaccgtcagactataaacataggc |
| | | agattgaaacaattcattacaagtaaaaattacagcattcttgaaaatatacctaatgagtccaag |
| | | gtactcttacgagagtctataaaactagacagatatgatgtactgacaagatctttaaggtattta |
| | | agagttgcaaaagaatcaacttcacttgagcaaaaattgctgggcgtatggatagctcttgaatgt |
| | | attttcgagagcacatcaggtaatatcatttctggaataactaaccatatccctacgttctatagc |
| | | actcaaagtctagaaattagaattagatattctaaagatttattagaagcccgattgaagcctatt |
| | | tcagatagccttttagagattacagccaatcagaaatctaaatttcgagacctttctttaaaagaa |
| | | tactttgacatagtgaaaatcgaaaaaaacaggaataaaattttcgatgagttagtttccaagggg |
| | | gatgagtttgccgtttttcgactaataaaaatatttgaatcattcggaacgtcaaagaaaataaat |
| | | gatagatttaatgatactaaaaaggatgttgagtctcagctttatagaatttacaaggtaagaaat |
| | | aaaataacccatagagcatactacggaaatattaggccccaattagtggatcatctttatagctat |
| | | ttactaagtgcatatagcacactaatttatagtttaagatataatgcaataaataaatttgaacca |
| | | caagatatgtttaatgcatatattatctcgtgcgagagtttaatattcaatgttgaagaagaaaaa |
| | | aaacttgaaaatataactatggatgaaataattttatcatagtgaatgttttctaggtgtcgtatt |
| | | c (SEQ ID NO: 283) |
| 33 | 12 | atggtagcgataaaaatgtatccggcaaaggatggggatgcttttcttattatttgcgatgaggaa |
| 34 | | aaaagtgcatttctgattgacggaggctacgcggaaacgttcaggcaacatattttgcctgactta |
| 35 | | cgtgagctgagttttaacggttaccggttacgtctggtcatggcaacacatattgattcagatcac |
| | | attggtggtctcgtggacttctttcttgtaaatggacacgcagcagagcctgcagtgattactgtt |
| | | gaccgcgtatggcacaacagcctcagggcgatgacgagacccgaaaataatgcacaaaaagtggat |
| | | tcccgagaaatcactgactttttgagacggagatatcatgtcgaagccgataaagccaaaccgcat |
| | | gaaatcagcgcgcgtcaggggagttcactggctgccagccttctggctggcgattatcattggaat |
| | | gagggaaaagggtatcagtgtatctgcaccggtacctccattcccaacttgatgtgcgataacagt |
| | | ctaacaattctgagcccctctaaggagagaatttcagcgctctgcctgtggtggcgcagacaactt |
| | | gcatcgctgggcttttcgggacggtcctcctcgagtgaggcatttgatgatgctttcgaatttttt |
| | | tgtaaaagggaagcatctcaggttcctcttccgcatgtcatcaatgcaagaacaccgttgcttgag |
| | | agggattatgcacgggatacctcgccaacaaatggcagttcgatagcgttcagtctggtgctcaat |
| | | aagaagagaatattgatgctaggagatgcctgggcggaagaagttgtgacatctctgggtgccagt |
| | | ggggcgtcccatcattttgatatcattaaaatctcacatcacggtagtattagaaacacaagcccg |
| | | aatcttttaaagatcatagatgctcctgtgtacctgatctcaaccgacggaaaaaagcatgccaga |
| | | caccctaacctggcggttctgaaagcgattgtggacagacctgcggcgtttacgcgaacgctctat |
| | | tttaactatgccaacagcgcatctgcttttatgaaaaattacctttctgcaagtggtgcacaattc |
| | | agaatcattgaaggatcaacggattggataacactgtgagatatgctgctactgaaactgaaataa |
| | | ggaacgcaactgtactcattgaatgcgcgggttacactggttccggaaccctgatcgcagcagaca |
| | | aggtccttacggctgcacattgtgtagtatcggatgatcctgagacaccaattacagtgacatttt |
| | | ttggtgcggatgaagacgtctgtgtcaatgcgacaatttcagaaatagatacatcgtgcgatgcct |
| | | gtctgctaacactttctgactctgtcgacattccgcctattacacttatgacacagccggagcgag |
| | | agggaagccaatggaaagcctttggctatccggcatcacgcaatgggccatcacattatcttcatg |
| | | gcactataagtcagattttaccaaggcttttccatggcgttgatatggatttgtcggtcagtgccg |
| | | attgtgttctggaagagtacagtggagtttctggtgccgccattctatcagaaaataaatgcattg |
| | | cgatggtgcgcatcaggatggatggtggactaggtgcagtaagtcttgataagttaagcggtttgc |
| | | tgattcgaaacggcctcatcccagatgacattgcatccctgccagattcatcactgtcgggtgaag |
| | | ttgtcctgaaccgcacagaatttcgcgacaactttgaatcgttcgtcctggagcacaagggacgtg |
| | | cagtgcttttggaaggtagtcccggctctggtaagactaccttctgccgccattatcagccccgta |
| | | gtgagcaactcgcagtggcgggtgtctatgaatttacaccggaagacggtgctggtacgacattca |
| | | aaattcttcctgaggtatttgccgattggctgcataaccaggtttctatactgctttcaggtaggc |
| | | ctgctcgcagggaggaaacagaaaagatcaatctgacccaaaaggtgtctgaccttctacatactt |
| | | tctcagattactggaagcacaaaggaaaatatggcgtcattttcattgatgctgtgaatgaggcaa |
| | | gcgagtgcggggatgaggcagtatcgcgctttacagcattactgccggtgacacttccggagaacg |
| | | tcaaacttgttttcaccgcaccatcattatcatcagctggtaaggctttccggcactggctcacac |
| | | ctcaggattgtatcagcctaacgcttttaagccatagggaggtgttacagctaacagctcgagagc |
| | | ttaaaacttccgccccttctttgtcactactcacacgagttagtgatatagctcagggccatccac |
| | | tttatctccgatacattcttgggtatctgaaagcgaatccggatcaggttaatctggagatattcc |
| | | cggttttcagtggcagcattgaaacctactacgaaaggctctggcaggggctggttaaggatgaga |
| | | gcgctgtaaatctgctcggtattctctcgcggatgcgctggggcattgatatttcatcactgatcc |
| | | ctgttctaacaccgcaggaacagacggtgtttgttccaacccttgaccgtattcagcatctgcttc |
| | | ttaatgataaatcatcagcattgtgccaccaatcatttgcggcgtttatcaacagtaaaacggcgg |
| | | taattaactcgctgctgcacggacgccttgccgacttctgccttaccagtggagagagttatggcc |
| | | tgattaatcgcgcttatcacctgctcctagcctctcacgacagacatcctgaagccgcattggtgt |
| | | gcacgcaggaatgggctgacgcctgtatcgtcaagggggctcagccggatattctaattcacgata |
| | | tccgtcagaccctgaagaacacgcttattcgtgccgatgcagtggcatcgattcgtctgttgctgc |
| | | ttttccaacgcatgaccttcagacaccattttttgtttctgcagtcagcttatcactcaggccttg |
| | | ccctggctgcacttggcagaccggatgaggcccttgagcagctcataccatctggaagcctcgttg |
| | | ttgatgcagttgatgcaattgtcagcgcacagactctcgcgcgtatgggaaacagtgaacacgcgc |
| | | tgaagctattggaaaaggtgaagtcagctgtcgaccaagaatttgaacgcaatcccgtcaatctat |
| | | ctgattttatcggcctttccctggcttgggtgagagctgagctgatggctggggtggttgatggcc |
| | | acggacgcacacgcgaggttgttgagtatttgtacggttgtgggcaagtcgttcgcgataattttg |
| | | aacaatcagcgcatagtaaatcagcatatacacgcgctttttatcctcttcaggcagaaatggaag |
| | | ccgtgaacatagcctttaatgaccgctccgtatctttacggacggttaaagaaaagtttggtagct |
| | | taccggaaaatattcttgatctgatgctcagttcagttatgcgggcacatgacatcattctgcaac |
| | | atcagttgccgatgccccagcatgctttgcaacccgtttggtacaatctggacagattacttcata |
| | | ctgatattccgtattcgaacgaaattcgttttaattcattaagtagccttatttttttcaatgcgc |
| | | cttctgctcttattatcaggatggcgggggtattttctttcgaagtagtacccgaaataacgttgc |
| | | tcaatgaagaaaatgagatagcagcagacagcattgacgttagtgaacagggacaactctggctgg |
| | | tgagcgcctaccttaatgaaacgcaaccctgtcccgatattaaacatccgagtcagggatgttctg |
| | | aatggctcaagacattgactgaggctattttttggtacagcgggcaggcgcgccgggcagttattg |
| | | acggcaacgatgagaaaaaagaactgcttttagtcaaggtgcagaatgatattctccctgctcttt |
| | | cgtactcgctggaagagcgcatggcatggccgaattcatgggcaatgcctgaacagattatcccca |
| | | tgatttacgaagagttagtaaacatgttcggcgcatgctggcccgataagatatcagtgatcactg |
| | | atttcattctggctcatacgcctcagcaatgtggactttattccgaggggtacaggcgtttactga |
| | | acagagttattcagactcttctaaatgagcatcggtttttggggcaatctgatacgacatttcaac |
| | | tacttgagacgttgcatgcgtttgtttctgcttttactgagaatcggcaggagctggttcctgaat |
| | | tactgaatattattccagcttatattagccttgatgctcctcagctggcacaggacacttacactg |
| | | agcttttaggtgtgtcgatgggccctgactggtacaaagaagaccaatttgccctcatgacaacta |
| | | tgctgcgcgtgataccacagcatacagacacaaatactacactttcacaagttgcaggattccttg |
| | | aacatgcttcgggtgaaatgacatttaggcgttatgttaggcaggaaaaatcacagtttattggcg |
| | | aacttattcgtcgtgggaattatgcacacgggtttaactattatcgtcagcagtcctgcggatccc |
| | | atgaggaaatgctcacccaacttagccacccagctgcagatagccctcatccattgaaaggcatgc |
| | | ggttcccggggggagcgctggatgaggaacatgctgtagaatgcattgtcagtgaactgcgaaaca |
| | | gagtcgactggcggcttcgctggggacttcttgaaatattcagctttggcagtattggtaatcttg |
| | | cagtgccctttgctgaacttatcaatgaattttctgcagacactgaagaccttaatgaaataccca |
| | | aaaggttgcacaacattttacatggtgatgtgcctttctcagaacacagaaattttatcaaaaatt |
| | | tcacagagcaccttgcagacaaccataagccactctttgctgaatttatcagtttgctatccgaag |
| | | acactagcgataacgacgttaagcctcccccctctggtgatgctaaccagaagggtactgatacct |
| | | cagatgatgtggcaatgcagccaggactttttgggaagcgttctgcgatcaatagggctgaagcct |
| | | gcatggaaaatgcccgaaaagccgcagcacgcagaaacacagttcgtgcaagtgagttagccgttg |
| | | aaagcctgcatataattcaggatggtgactggtcagtctggagaaagaacaaccatctggcggaac |
| | | ttacacggacgtacatattggacaactctgcggatgcaggttcggtcattcgtgcttatgcttcgc |
| | | ttgtagaaaaagaacgttatgccccggcatgggtaattgctagtcatctcatcgaaatagcagcca |
| | | gtaaattctctgatcaagaagcccaagctattaaccagatcgtacttgaacacaaccgccacatgc |
| | | ttgggaataccgaagcggatgctgcgcatttttcttttcttaatgaacctgatacctcagatgcag |
| | | gtgaagaaacactctattttctgttttggctgctggaacacccactgaaattcagacgcgaacggg |
| | | ctctggaagtactgaagtggcttgcatcagacgatgataagattctgggccaatgcgtgacggagg |
| | | cactcgtttcagacattgcctcacgagctgaagcactaatggcattgacagactgggtgtcagcta |
| | | gatctcctcagcgaatatgggactttatagttaaagagcgcagcctttttgaatggcttgaaggca |
| | | ctactgcactaagccaagtccatctcctggagcgagtaaccagcagagcgggatttgttttaagaa |
| | | atgagattgccgcatttgagcgaccccgaaagcttttactgacatcagaagcctctggacaacgga |
| | | atattccagaaaatttaccaacatgggtgcaatccttgtcgcagacccttgccgtgatggaaaagc |
| | | agggaatagatatcccagctttgcttaccttactcgaaaaacgggttttacagcagagtggattgg |
| | | ctgatatcacggtggcttttgagctggaaaagttacttgcgcgtggttttactgtgaatagaacac |
| | | caagtcaccatcgctgggagacgatggtgcgatttgcattaaaccagatcatacatgaggcggccg |
| | | cacaggatgaactgcaaaacattgaacccttgctacgtgcctggaaccccgcgtcagaggagtgtg |
| | | ttgagccgtgggaggtttgtaaccgggcaaaacagattatctgcgctgttatggaaggtagacatc |
| | | agcaagcttcgggcatagaggatggctttttcttgcattatcttgatgaagtggaggtttcccgag |
| | | aaggtcaaacgcatctggtggaaatctcagcggtgttaacgacagctcataatggtcatgagagcc |
| | | ttagaccaggtgcagaaagcgaatttaatgcaacacagacacctgatatagagcggacgcttagtg |
| | | tgcaccttacatgccagcgagtcaaaatgcagcctttgctttttgggggagctacgcctgccgcag |
| | | tgtcgaaaaagtttatgcagatgactggaacgttgccttcagactttattcgcaggcaatggcgaa |
| | | gcgggcgttctcttagtaaaaacagatggggggaaccaataagcagaggaagtctgttactcatga |
| | | aaagaacaactaccctccctccaggactgggcttagcgtggtatgtcactgtcgatgggaagttga |
| | | tgaatatattttcatatgccccgaggaggagataatgaaatacagttcaatggaaacgccaaaaac |
| | | gcgagaggaatttgaggctcgctgttttcacctgctcaatgcgatcaagttaggacggtatcatgg |
| | | cattccgggtgaaggtaacaaagagcaggttccttttctccctaacggacgagttgatctggcaaa |
| | | cattgataccatgactcgcctctcgatgaactcgttatatgatttccactataacagggataatta |
| | | tccgcagtttgatctctctgaaaatgacgagaatgaagaggctacggattga |
| | | (SEQ ID NO: 284) |
| 36 | 13 | gggatttccaccacctcccaccgaccatctaagactttatgccactgtccctaggactgctatgta |
| | | ctaggagcggatgttaaactcagactcgtttcagctacattgcgttttgaataatattccatcata |
| | | ataactctttgaaaaatgtgatcttttcatttataacactgatgacttgcttatctcattgggata |
| | | tcggaggagaatacttaactatgacaagcccgattattatgacactggctatattatatagattga |
| | | tattaaaatgtaggattaggttcttgccaaggtgtcaagatttacagataggtttaaaaccatata |
| | | aatatgttttacggtgagatacaatacatattgtaaggcataaacgcttggtaaaattttaattat |
| | | tggaagaagctaatcatggaacccatatcaattacagtggcaacttatgtagcaactaaacttatt |
| | | gatcaattcatctctcaagaaggatatggttgtattaagaaagcattattcccccaaaaaagatat |
| | | gtggatagattatatcaactaattgaagagacggcaattgagtttgaagaaacatatccagtagaa |
| | | agtggagcaataccattttatcattccgaaccattgtttgagatgttgaatgagcacatctttttt |
| | | aaagagttccctgacaaagagatattattagacaagttcaaagaatatccaagtatcactccccca |
| | | actcaacaacaactcagccttttttatgagatgttatcattaaaaatcaataattgttcgaagtta |
| | | aaaaagctacatatcgaagaaacgtataaagaaaaaatattcgatattaatgaagagctcattcaa |
| | | gtcaaacttattttacggtctatagatgagaaactaacttttcacttaagtgatgattggttaaat |
| | | gaaaaaaatagtcaagcaatagctgacttgggaggtcgatacacacccgaactcaacgtaaagcta |
| | | gaaatagcagagatatttgatggcctcggtagaactaatgatttttctaaaatattttattcgcat |
| | | atagatagctttctggtcgctggaaagaaattacatagttgcgatgtaatttcctcagaattattt |
| | | gaaataaaccagtccttaaaagaaatttctgatatatatcaggagattaatttttctaaattagat |
| | | gaaatccctataaataaatttaataactatgtttctagctgccagacagctattggcggagcggta |
| | | tcaatattgtgggaactccgagaaaagtcagagcaagtaggtgaaaccaagcattacagtgataag |
| | | tattcatctactctgcgaatgcttcgggaatttgactatgcgtgcaatgaattacgtatattcatt |
| | | aattcaacaacagtgaagttggctaacaacccattcttacttctcgaaggaaaagcaggaattggt |
| | | aagtctcatttactggctgatgtgattaaaaatcgaattgcttctgggtatccttcactactcata |
| | | ctagggcaacaacttacttcagatgaatctccatggtcacaaatcttcaagagattacagcttaaa |
| | | atcacttctcgtgaattcctagaaaaactgaatttatatggcaaaaaaacaggaaaaagagtctta |
| | | gtttttattgatgctattaatgaaggtaatggaaataaattctggaatgacaatattaacagtttt |
| | | gtcgatgaaatcagatgctttgaatggcttggtctgataatgtcagtcagaacaacatatagaaat |
| | | gtaacaatttcacatgagaatgttgtgcgaaataattttgaaattcatgaacatattggattccag |
| | | aacgttgagttggaagcggttagtctattttatgattattacaatattgagaggccttcatctcct |
| | | aaccttaatccagagtttaaaaatcctctatttcttaagttattgtgtgaaggcattaagaaaaat |
| | | ggtttaaccaaagtgcctgttggatttaatgggatttcaaatatttttaactttttagttgaaggg |
| | | gtaaataaatcattagcatcgccaaaaaaatatgcattcgatcccagttttcctcttgttaaagat |
| | | gctctcaatgaaatcataaaattcaaattagagattggtcgtaatagtatttcacttaaagatgct |
| | | cactcagtggttcaatctgtagttaatgattatgttgctgataaaaccttcctcagcgccttgatt |
| | | gacgaaggattattgactaaaggcatagtgagaaatgatgataattctactgaggaagtagtttat |
| | | gtggcttttgaaaggtttgatgatcatttaactgttaattttttattaaatgatgttgaaaatatc |
| | | gaaagtgaatttaagcctgatggtcgtctgaaaaaatattttcatgatgaatgtgatttttatata |
| | | aaatcgggaatagtagaggcgttgtctattcaattgccagaaaggtatgaaaaagagctttatgaa |
| | | tttctgccggagttcagcaataatcttaaattactagaagcctttattgatagcttgatatggcgc |
| | | gatattaaggctattgatttcgaaaaaattagacctttcatcaatgaacatgtttttaaatttaaa |
| | | gatagttttgatcatttcctcgaggcagtgatctctatttcaggtttagttggccatccctttaat |
| | | gctaatttcttgcatgattggctaaaagattattctttggcaaatcgagattcgttttggactaca |
| | | gaacttaaatataaatatagtgaagactcagcatttaggcatctaatcgattgggcatgggccaga |
| | | acagataaaagctttgtttcggatgagtcaatcgagctagttgcaactagtttatgctggttttta |
| | | acttctagtaaccgagaacttcgagattgctcaactaaggctttagtgagtttactcgagccaaga |
| | | attcctgtattgagaaaaataattgataagttttatggtgtaaatgatccttacgtttgggaaaga |
| | | atatttgcagttgcattaggctgtacattgcgaactgataatattaaagaactaaaatatttagcc |
| | | gaaactgtttaccaaaaggtattttgttctaagtatgtgtatccaaatatattacttagagattat |
| | | gctagagagattattgaatttgctaatcatcttggattggaacttgaaagcattgaattatccaag |
| | | actagaccaccctacaacagcatttggcctgacaagattccttcaaaagaggaactagagtccctt |
| | | tatgataaagaaccttatcgggaactctggagctctattatggaagatggtgacttttcacgatat |
| | | actattggaacaaattataatcattctgattggtctggttgcaagtttaatgaaacccctgttgac |
| | | cgtaagcaagtttttaaaactttcaaatgtaaactaactgatcaacaaaaagacttgtatgatgcc |
| | | acagatcctttcatttatgatgataaatgcgaaggaattaaatttggtcgtgtggtcggtagaaaa |
| | | gcacaggaagaaataaaggcgagcaagaaattatttaagaattcattgtcatacgatctgttaagt |
| | | gagtttgaaaatgaaatagagccatacctggatcataataataatctgctggaaactgataaacac |
| | | tttgatcttcgactagctcaacaatttatattcaatcgtgttatagagcttggttgggatccggag |
| | | aagcatggtaattttgaccaacaaataggaactggacgtggacgtagagaggcattccaagaacgg |
| | | attggtaaaaaataccaatggattgcttattatgaatacatggcaaggctagccgataattttact |
| | | cgttttgaaggttatggtgacgaacgaaaggaaaatccataccaagggccatgggagccttacgta |
| | | agagatatagatcccactatcttacttaaagaaactggaacgaaaaaaataagcaataaagaaatg |
| | | tggtggcttaatgatgaagtgtttgattggacttgctctaatgaagactgggttaaaagttctact |
| | | actataactaattcatatgcttttattgaagttaaagatgataatggtgatgaatggatagtatta |
| | | gaaagtcatccatcatggaaagaaccaaaaattattggaaacgatgattgggggcacccacgaaaa |
| | | gaggtttggtatcagatcagaagttatatcgttaaagttgaagaatttgaaaattttagatgttgg |
| | | gcaatagctcaagactttatgggcaggtggatgccggaatgtactgatagataccaattatttaat |
| | | agggagtactattggtccgaagcatttaagtcttttaaatcagattattatggtggatctgactgg |
| | | acttcggtaacagaccgggagtctggagctaagatagctgatgttagtgtcacttcgattaattat |
| | | ttgtgggaagaggagttcgacaaatcaaaaatagaaactttgaattttttgaagcctagtaactta |
| | | atctttgaaaagatgggattaaaaagtggggaagtagagggtagcttcaatgatgaaaatggaact |
| | | atggtttgctttgcagctgaagctgtatatgcttcaaagccgcatctacttgttaaaaaagaacca |
| | | tttttaacaatgttaagggacaatggttttgaaatcgtttggacattattaggtgaaaagggcgtt |
| | | atagggggctcactcatatcaagtcatcattatggtcgacaggagtttagtggagcattttattat |
| | | gaagacagtcagctaacaggaagtcataaaactagctttacgagataaaaatgaatctcagagctg |
| | | aatatataagtagtattagaaaccgggttatacttaagaaatcaatcttaagtgtggcagtcgaat |
| | | ggtagctaatatgctagcggcgctaatgcctgtttgttgctcataacaggcattcactttagttat |
| | | ggcagaaaagtatacatgctgggttgggaaagtgtgaaagaaaggaagattgctgcgccgtttgtc |
| | | gtcacgtttatcttcattggctatgca (SEQ ID NO: 285) |
| 37 | 14 | acaattttttgccataagacgctttcctgaaactcttctcattctcagcaggaaagcgttctcttc |
| 38 | | tcaatactctctggttatagagtattaaaaaataaggagttataatccttgtagcccaactgacat |
| | | aaggacgatgctcaatgtctgacagcctgcttgttcgcaccagtagagatggcgatcagtttcatt |
| | | atctttgggcggctcgccgcgcccttcgactactggaacctcagtcaactcttgttgccctgacca |
| | | ttgaaggggcatcaacgacggaaatgggctctcagccagtggttgaggatggggaggagctgattg |
| | | atattgctgaatattacggcagtaacgagctcgcaacagcaacaactgttcgttatatgcagctaa |
| | | agcattcaacaatgcactcagatactccatttccccctagtgggttacaaaaaaccatcgaaggtt |
| | | ttgcaacccgttataaggcacttatacaaaaaataccggtagaaacgttacgcactaaactcgagt |
| | | tctggtttgtgacgaaccgtccagtcagtagcagcttcagtgaagcgatcaatgatgccgcgaacc |
| | | aacacgttacacgccatccacatgatctggcgaaacttgagaaatttaccgggcttcaaggcgctg |
| | | agttatcgatattctgccagcttttacatatagaaggtcagcaggacgatttatggagtcagcgga |
| | | atatcctgctaagagaatcagcgggatatctccccgacctggatactgaagcccctctgaaattaa |
| | | aagagctggttaacagaaaagcgttaaccgaaagcgccgcaaatccttccattaccagaatggatg |
| | | tgttgcgtgctttgggggtggatgaaacagatctttttcctgcgccctgtcgtattgaaagaatag |
| | | aaaattccgtctcaagaactcaagaggcgacgctggttcaacgtgttgttgaagcattcggcgcac |
| | | ctgtgatcatccatgccgatgccggtgtggggaaatcaattttctctactcatatagaggagcatc |
| | | ttcccactggttctgttagcatcttatatgactgtttcggactgggtcagtaccgtaacgcgtctt |
| | | cctaccgccaccaccatcgtacagcattggttcagatggctaatgaaatggcatctcgtggtctct |
| | | gtcatccattgatcccaaatgctggtactggcatatcccagtatatgcgtgcgtttctgcatcgcc |
| | | tttctcagagcatttcaatactccgggcctctgagcccttggccgtattgtgtattattattgatg |
| | | ctgcggacaatgcacagatggcggcggaagaaatcggtgaaacgcgttcttttatcaaagatttaa |
| | | ttagagaaaagcttcctgatggagtctgccttgttgcactttgccgaccttatagacgggaattac |
| | | ttgatccacctcctgaagcactcacattatccctacaaacttttaatcgcgatgagacagccgctc |
| | | atcttcaccaaaaatttccagatgccagcgaaagtgatgttgacgagttccatcgtctaagctctt |
| | | gcaacccccgggttcaggctctgtcattatcacaaaatcttccactgaacgacacattgagacttt |
| | | tggggccaaatcccaaaacggtagaagatactattggtgaagtgctggaaaaatccattgctcgct |
| | | tacgtgatacagccggaatatctgaacgtgctcaaattgatacgatttgttccgcactggcaatat |
| | | tgcgtccattaattccattatctgtgctatctgccatttccggagtagctggttctgctattaaaa |
| | | gtttcgcacttgatctgggacgcccgttaatcgttagtggcgagactattcagttctttgatgaac |
| | | cggccgaaacatggtttcagaggcgctttaggccatcggccgctgatctgcatcagtttattacta |
| | | aactgagaccactaacaaaagatagttcctatgcagcatcagttttacctgcattgatgctggaag |
| | | gaaaccagctttctgaactgatcgagctagcgatatcctcacaagctctgcctgaaaccagcgcgg |
| | | ttgaacgcagggacatagaacttcaaagattacagtttgcgttaaaagcagccttacgcacaggtc |
| | | gataccaggatgcggctaaactggcactgaaagctggtggagaatgcgcgggtgacaacaggcaaa |
| | | gagtcctgctgagggacaatatcgatctggcagcaaaatttgtgggaagcaacggcgttcaggaac |
| | | tggtttcccgtaacgcatttccagatactggctggcctggctccagaaatgcttattatgccgcaa |
| | | tactttccgaatatcctgaactctcaggagaggcccgcagtcgccttcgactcaccatggagtggt |
| | | taacaaactggagtcaattaccagatgatgagcggagcaggcaaaatgttaccgatcaggacagag |
| | | cggtaatgctcattgcctgcctgaatattcatggcgcggaagcggcagcaagggagctcagaaggt |
| | | ggcggcctcgaaaactatcttttgacgctggaaaaattgttgccatgcagttactggcccacgccc |
| | | gttatgatgaacttgatcagttggctattgcggctggaaacgatatcagcctggttatgggaattg |
| | | tactggaagcaagaaaacttcaccgtccagtcgctgaacaagcaatcagaagaacctggcgcttgt |
| | | taaaaagtcagcgagtcagcattaaagacagaaaccacgctaataaccagacaatagcagcaatca |
| | | ctggcatggttgaaatggcgcttatccaatctgtttgtactgaatcagaaagcatccagttgttgg |
| | | atcgttatttaccaaaggttcccccctatgctctgacttctgagtatagtaaagaaagagttgctt |
| | | acgtccgggcatatgctctgcaggcaaacctgatgggctctcaattagcgcttagcgatttagcct |
| | | ccacagaggttaaaaaagaacttatggctgaaaaacgccacggcgaatctgatgacctgcgtcaac |
| | | tgaagcagtacagcggagtattaatcccttggtataatttatgggccaaagtaattcttggtaaaa |
| | | caaggaaagcagacttagaaagtgagctaagtgatactcaaaaagaatcgacggctattaaaggtc |
| | | attcttactctgagcattcattatcatcaaatgagatcgcaaatgtatggtttgatattctgatcg |
| | | aagcaggtaatgtatcaaaagacgatgtggaaaacatcatcaaatggagtcagcataaagggaata |
| | | gagtattcacaccaacgcttcaccgtttcagttctgtatgtgcagagatttcagggcttggagagc |
| | | tttcatatcacttcgcagaacttgccttatctttatggagggatgagcactctgatgctcagatca |
| | | aagctgacggctatatagacctttcccgttcactcatttcacttgatgaaccagaagctaaagaat |
| | | actttaaccaagcgattgaagttacaaataagttaggcgatgaaaatttaagtcgatgggaagcga |
| | | tacttgatcttgctgaatatgttgctggtaaaacgcaagtccctcctgaaatttcctataaactag |
| | | cccgatgtgcggaactaaccagagaatatgttgatcgtgataaacattttgcatggagtgatactg |
| | | ttgagattttggctgagttatgtccatcttcagccctagcaataataagtcgttggcgtgaccgta |
| | | catttggcaatcatagaagcatactggcatggaccattgagcatcttgtaaagaaaaataaaatta |
| | | atgcactcgatgcacttcctttaatcacatttgagaatgattggcataaatgcgacttgcttgatt |
| | | cagttttatcctcgtgtactgatgacaaagataagatcatggcattcgaagtggtttaccactata |
| | | caaaatttaacgtacaaaatatccaaaatcttaaaaagctggatgctatttctacatcattaggta |
| | | ttgaacacacagaactgaaagaaagaatttcaggtctacaacatactgagacggtttcaaaaaaat |
| | | ccagtctctcatcgaatgataatgagcaaggccatgaccaggaatgggagtccatttttaaagatt |
| | | gtgatttatcgtctattgatggtattagtgcagcatacgaaaaatttcgtaatgttcctgaattct |
| | | attccaaagaaaccttcatcaagaaagcaataagccgagttaagacgggcaaagaatgtagtttca |
| | | ttactgccattggtgctatatttcactgggggctttatgattttaaatatattcttgaatctatac |
| | | ccgacgaatggacatctcgtttaagcattaaaaccaccctggcaggtttaataaaagaatattgcc |
| | | aacgcttctgtatgcgaatcagaaaaagtcgcgtttacgagatttttcccttcagtctggccagca |
| | | ggctttctggtataagtgaaaaagagattttcggtattaccctggaggccattgcagaatcgccag |
| | | agcccgcaaactctgaccgtttatttagccttcctggccttcttgttagtaaactggagagtaatg |
| | | aagcgttagatgtattatcttatgccttggatttattcgacgaggtgctaaaagatgaggatggtg |
| | | acggcccatggaacgagaaattatctccgccaactcatgtagaggattcacttgcaggctatattt |
| | | gggcgcggctgggttctccggaggcggaaatgcgctggcaggcagcacatgcggttctggcactat |
| | | gtcgaatgagtcgtacatgcgttatacaaggaattttccagcacgcaataaatgctaccactttac |
| | | ctttttgtgatcgcaatctgcccttttataccctccatgctcaattgtggttgatgatcgctgctg |
| | | caagggttgcgctggatgatggaaaatcgctgattcccaatattggttatttctaccattatgcca |
| | | ctactgatcagccacatgtattaatccgtcattttgctgccagaactttacttgcactgcatgata |
| | | gcgacctgatctctatcccagcacaagaagagaataaactccgaaatataaaccagtctacgactc |
| | | tccctgtgcttgataaggttgaagatcatagaggtgaagattcatatacttttggtatcgactttg |
| | | gcccttactggctaaaacctctgggacgttgtttcggtgtatctcaaaaacagttagaacctgaaa |
| | | tgcttcgcattattcgtgatgttcttggttttaaaggtagccgcaactgggatgaggatgagcgta |
| | | ataaacgacgctattatcaagacagagataatcatcacagtcatggttcctatccacgggtcgatg |
| | | actaccatttttacttgtcataccatgcaatgtttatgaccgctgggcagttattagcgacaaaac |
| | | cattagttggtagtgactacgacgatgtcgaggatgttttccaggactggttaagaagacatgata |
| | | tttctcggaacgatcatcgctggctcgccgatcggagagatattccccccaaagagcgctccagtt |
| | | ggcttaatagcagttctgacaatagggatgaatggctagcgtcaatctctgaaaatgtatttaacg |
| | | aaacactatgtcccagccccggactattaacgctatggggacgttggtctgacgtttgttcagatc |
| | | gaaaagaatctattattgtccattctgcgttagtatcgccggagcgatctttatcgctcctcagag |
| | | cattacaaacaactaaaaatgtatatgactataaaatccctgatgctggagataatcttgaaatag |
| | | atcacgcacactatcagctaaaaggatggattaaagatattgctgaatactgtggaattgatgagt |
| | | ttgatccctgggcaggtaatgtaaggtttccaatcccagaaccagcctcatttatcattgatgcga |
| | | tgaaattaactactgataaagatcatcgggtatggtattcaccttctgatgttgaaccggcgatga |
| | | tttccagtatctggggccatctatcaggtaaaaatgatgaggaaaaatcacatggttataggctat |
| | | gtgcttcaatacacttcataaaatcagcattagaaacattcaacatggatctcattttagaggttg |
| | | atgttgatcgctattcacggaacagcagatatgaacggaataatgaaaatgagctcgacaatatcc |
| | | cttcaagcactcgactcttcctcttccgacatgacggaaccatccacacgctatacggcaattata |
| | | gaaatggggaaaaaactagttgatgagcttgagctaaatgactctgttgatacattaagcagatgg |
| | | atggctcatcatatcgcagagctcatttatgatgctgaacattgtacagacgacatcgtccgtaca |
| | | gctaaacaagcggagattagggactctatctggtcattctggtctaacagatacgaattgccaatt |
| | | ggtagcagaccatttcaggagctcgaacctattctaagaaccttaaaaggtcttgatcctgaaaat |
| | | gagcaaccgagatttttttcaccttaccgagatctaattaatgtagaaaaagaaaccagtgaggtc |
| | | caaaaatggctaaccgccgctaaggatattgattcagcagcaaaaatactgattgattactgttta |
| | | tcgttagcagcagaaaatgctatcgataaatcccaagaatgggtggaattagcacagaaagctgga |
| | | ttgaacaaagatgttgatctgcttgaaattcgtatctttcagttacgaggtaccccagccaataca |
| | | gacaatcccaataatgcacaacggagaatactggaaaaaaggcaaaaaaggcttgaagcttttctc |
| | | ttattgggctcccagttaaacgaacaactcaaatctcagcttgaagccttaccagcaattgaggat |
| | | gagccaacggatgacgacgaagacttttgatatgacttgctttagcactggagacggctcacaaga |
| | | cggaccacataatagcctaacccaagacttttctactagtcctaatg (SEQ ID NO: 286) |
| 39 | 15 | gcgcagctgacaaagattgaccgtgagcgctctgatggagaaagacgatagttgctgagtacgata |
| | | tcgagggtacatttctctgtgtaggggtagttatttacaaaaaaataggagaataattaaatggtc |
| | | aaaccaaactgggataactttaaagctaaatttagtgagaatcctcaaggtaattttgagtggttt |
| | | tgctacttgttgttctgtcaagaattcaaaatgcccgcaggtatatttagatataagaatcaatct |
| | | ggtatcgaaactaatccaataaccaaagataatgaaattatcggttggcaatctaaattctatgac |
| | | acaaaattgtcggataacaaagctgatcttatagaaatgattgagaaaagcaaaaaggcttatcca |
| | | ggattaagtaaaatcattttctatactaatcaagagtgggggcaggggagaaagtcccatgaacct |
| | | gaaggcgataagaacgctgataattatttggaaactgtcggaaatagtaacgatcccaaaataaaa |
| | | attgaagttgatcagaaagcatatgagtcgggtatcgaaatagtatggagagttgctagttttttt |
| | | gaatcaccgtttgtaatagttgagaatgaaaagattgctaaacatttcttctcccttaatgaaagc |
| | | atctttgatttattagaagaaaagcgcaagcacacagaaaatgttttatatgaaattcaaaccaat |
| | | atagagttcaaagacagaagtattgaaattgacagacgacattgcatagaacttctacatgagaat |
| | | ctagttcagaaaaaaattgtcatcgtcagcggagaaggtggggttggaaaaacagcagttatcaaa |
| | | aaaatttatgaagcagaaaaacaatacactcctttctatgtctttaaggctagcgagtttaaaaag |
| | | gacagcattaatgagttattcggtgcgcatggcttagacgatttctctaatgctcatcaagacgaa |
| | | ttacgtaaagtcatagtcgtagattctgctgaaaagcttttagaactgaccaatatcgatcctttt |
| | | aaagaattcctgactgttttaataaaggataaatggcaggttgttttcacaacccgtaacaattac |
| | | ttggcagatctgaactatgctttcatagatatttataagataactcctggaaacttagtaataaag |
| | | aaccttgaacgcggcgagctaatagagttatctgataacaatggatttagccttcctcaagatgtt |
| | | cgattattagaactaatcaaaaatccattttatctaagtgaatatttgaggttctataccggtgaa |
| | | agcatcgattatgtgagcttcaaagaaaagctatggaataagattatcgtcaaaaataaaccttct |
| | | cgggagcagtgtttcttagcgactgcttttcagcgggctagtgagggccaattttttgtctccccg |
| | | gcatgtgatactggaattttagatgagttagttaaagacggaattgtcggctatgaagctgctggt |
| | | tacttcattacacatgatatatacgaggaatgggcattagaaaagaaaatttctgtcgattatatc |
| | | cgtaaagcgaacaataacgagttcttcgaaaaaataggagaatcacttcctgttcgccgtagtttt |
| | | cggaattggatatctgaacgattgcttttagatgaccagtccataaagccttttatcgcagaaata |
| | | gtctgtggagaaggaatatcaaatttttggaaagacgagttatgggtagctgtccttctttccgac |
| | | aattcaagcatattttttaattactttaaaagatatttacttagtagtgaccagaatctattaaaa |
| | | agacttactttcttattgaggcttgcttgcaaggacgttgattacgatctgcttaaacagttaggt |
| | | gtaagtaattcagatctgctttccattaaatatgttcttactaagcctaagggaactggttggcag |
| | | agtgtgatccaatttatctatgaaaatttagatgaaatagggatcagaaatattaattttatactt |
| | | cctgtgattcaggagtggaatcaaagaaacaaagtgggtgaaacgactcgattatctagtttgata |
| | | gctctaaaatattatcaatggactatagatgaggatgtctatttatccggaagggataatgagaaa |
| | | aatattctgcatacgattcttcatggggcggccatgattaaacctgaaatggaagaggttttagtt |
| | | aaggttcttaaaaataggtggaaagagcatggtaccccatatttcgaccttatgaccttaatcctt |
| | | actgacttagattcatatccggtttgggcatctctcccggaatatgttctacaattggcagatctg |
| | | ttctggtatcggccacttaaagaaacaggcgaacgttatcacagtatggatattgaagatgagttc |
| | | ggtctatttaggtctcatcacgactattatccagaaagtccatatcagactcctatatattggtta |
| | | ctacaatcacagttcaaaaaaacaatagactttattcttgattttacgaacaagacaacgatatgt |
| | | tttgcccactcccattttgctaaaaacgaaattgaagaagtagatgtctttattgaagaaggaaag |
| | | tttataaagcaatatatatgcaatcgtctgtggtgctcataccgaggaacacaggtctctacctac |
| | | ttactttcatcaattcatatggcattggaaaagttttttcttgagaattttaaaaatgcagactcg |
| | | aaagtgttggaaagttggcttcttttcttgttaagaaataccaagtcagcttctatttctgcagta |
| | | gttacgagtattgtacttgcattccctgagaagacattcaatgtagctaaagtactattccaaaca |
| | | aaggacttcttccgttttgatatgaatcgaatggttctagacagaacacataaaagttcattaatc |
| | | tccctcagggatggctttggcggtacagattacagaaactctttgcacgaagaagatagaattaaa |
| | | gcttgcgatgatgtgcatagaaatacttatcttgaaaatcttgccttgcattatcaaattttcagg |
| | | agtgaaaatgtaacggagaaagatgccattgaaaggcaacaagtgctctgggatattttcgacaaa |
| | | tactataatcagcttccagatgaagctcaagaaactgaagccgataagacgtggaggctctgcttg |
| | | gcaagaatggatcggcgaaagatgaaaataactaccaaggagaaagatgaagggattgagatatca |
| | | ttcaatcctgagattgaccctaaactaaagcaatatagtgaggaagcaataaagaaaaactccgag |
| | | catatgaagtatgtaacgctgaaactatgggcaagctataaaagagaaaaggatgaacgttataag |
| | | aattatggaatgtatgaggacaatccgcaaattgctttacaagagaccaaagaaataataaaaaag |
| | | cttaatgaggaagggggtgaagatttcagactattaaatggtaatataccagcagacgtttgttct |
| | | gtattactgttagattattttaatcagttgaataatgaagagagagaatactgtaaagatattgtt |
| | | ctagcgtattctaaacttccgttgaaggaaggctataattatcaggtacaagatggaacaacctcg |
| | | gcaatttcagccttacccgtgatttatcataattatccaatggaaagggagactataaaaacaata |
| | | ttacttttgacactgtttaatgaccactctattggaatggcaggtgggcgctactcagtatttcct |
| | | agtatggtgattcataaattatggctagactattttgatgatatgcagtccctattgtttggtttt |
| | | ttgattttaaagccaaaatatgtaatcctttcaagaaaaatcattcatgaaagttatcgtcaagta |
| | | gactatgacattaaaaaaataaatattaataaggtgtttttaaataactataagcattgcatatca |
| | | aatgtcatcgataataaaatatctatagatgatttgggaagtatggataaagttgatctacatatt |
| | | ttgaacacagctttccaattaattccagttgatactgttaatattgaacataagaaattggtttcc |
| | | ttaattgttaaaagattttctacaagcctattgtcaagtgttcgagaagatagagttgattacgct |
| | | cttcggcagtctttcttggaaagatttgcctactttacgcttcatgcgcccgtgagcgatattccc |
| | | gattatataaaaccttttcttgatggtttcaacggttcagagcctatttcagagttatttaaaaaa |
| | | tttattctcgtcgaagatagattaaatacttacgccaaattttggaaggtttgggatttgtttttt |
| | | gataaagtggttactttgtgcaaggatggagataggtattggtatgtagataaaattataaaaagt |
| | | tacctttttgctgaatctccatggaaagaaaactctaatggttggcacacatttaaagatagcaat |
| | | agtcaattcttttgcgatgtatctaggactatgggccattgcccttcaactttatattctcttgcc |
| | | aaatctttgaataacattgccagttgctatcttaatcaaggtataacttggctttcagaaatattg |
| | | tcggttaataaaaagctatgggaaaagaaattggaaaatgatactgtttattatttggaatgtttg |
| | | gttaggcggtatattaacaatgagcgtgagcgaattagacgaaccaaacagttgaaacaagaggtc |
| | | ttagtaatattggattttttggtagagaaaggatcggttgttggttatatgtcacgggaaaatatt |
| | | ctgtgatgtagttgaaaataataattttaatgagagcttttccaatttaggctccagggattggag |
| | | cctttttattatcg (SEQ ID NO: 287) |
| 40 | 16 | actagctaagcaataagggcgatcggctctcccatagatcgaggccgaatgatgttagcaatgttc |
| | | actcttggctggaatctgccagaaatcgaggtcatatggtctgctttgagtgaggagcgcaaatgg |
| | | ataaagccctcatgagttctttttcaatgacctaacttttgagaggcactgggttagatcatgttt |
| | | catgtttgcaatacaatatatatttaaacttaggtttataacttaaatgttagttcctgatctaaa |
| | | ccagattattaatcactcctagagtgaaatgagttaagccaagagttgataaaattaacagttttt |
| | | tttacaatatctggatgtttgctagcgaacaggcatctaaaataactatgctgagctaaacttaca |
| | | attcaaattgtaccgaggataaaatgcaagtacaacatcatactgaaccaaacttgaagaatgaga |
| | | ttgtggctttatttaaggcttctcaattgatacctttttttggcagtggatttactagagatatta |
| | | gagcaaaaaatggtaaagttcctgatgctattaaatttacggagttgattaggaatatagcggcag |
| | | aaaaagaagggttaacacaaacagaaatagatgaaattctaagaatcagccagcttaaaaaagcgt |
| | | ttggacttctaaatatggaggaatatatacccaaacgaaaatcgaaggcattattaggtaacattt |
| | | tttcagagtgtaaactctctgatcacgaaaagacaaaaataataaatttagattggcctcatattt |
| | | tcacgtttaatattgacgatgctatagaaaacgttaataggaaatacaaaattctgcatccaaatc |
| | | gagcagttcagagagaatttatatctgctaataagtgtctattcaaaattcatggcgatattactg |
| | | aatttattaaatacgaagatcaaaatctgatatttacttggcgtgaatatgcacacagtatagaag |
| | | aaaataaatccatgctatcctttttatctgaggaagccaaaaactcagctttccttttcataggtt |
| | | gcagtcttgatggagagcttgatttaatgcatttatcaagaagcacaccatttaagaaatcaattt |
| | | atttgaagaaaggatatttaaatttagaagaaaaaatagctctttcggagtacggcatcgaaaaag |
| | | taattacctttgacacttacgatcagatatatcaatggttaaataacacacttcagaatgttgagc |
| | | gaaaatcccccacaagaagtttcgaactcgatgactccaagttaatgaaagaagaggctataaatt |
| | | tattcgctaatggaggccctgtaactaaaatagtggataataaaagaatcctgcgaaattctataa |
| | | ctttttctcaacgagatgtctgtgatgatgcaattaaagcactacgtaatcatgactatatcctaa |
| | | ttacaggtcgacgtttcagcggaaaatctgtacttttatttcaaattattgaggcaaaaaaagaat |
| | | ataatgcctcttattactcttcgactgacacattcgatccttccattaaaaactcattgataaaat |
| | | tcgagaatcatatattcgttttcgactctaatttctttaatgcacaaagcattgatgaaattttaa |
| | | ccacaagggtgcatcctagtaacaaagttgttttatgctcgagttttggtgacgcagagttatata |
| | | gattcaagttaaaggataaaaagatattacataccgaaattcagattaaaaataacttgattaatg |
| | | aagaaggtaactatctcaatgataagctttcttttgaggggctaccactttataaatcttcagaaa |
| | | cgttgttgaattttgcttatcgatactatagcgagtataaaaatagactaagtggttctaatttat |
| | | ttaataagcaatttgatgaagattcaatgtttgttttgattttaattgcagcttttaataaagcca |
| | | catatggtcatatcaacagtcacaataaatattttgatattcagaattttatttcgcaaaatgata |
| | | gattatttgaattggagtcaactaacacagatccaagtggagttataatctgcaattcaccatcct |
| | | ggcttttaagagttatcagtgagtatattgataagaatcctgcatcttataaaacagtatctgatt |
| | | taataatatctcttgcgtcaaaaggatttcttgcagcatcaaggaaccttataagctttgataaac |
| | | taaatgaacttgggaatggaaaaaatgtccataaatttatcaggggtatatataaggaaattgcac |
| | | atacctatcgtgaagatatgcactactggttacaaagggctaagtcagaattaatatcggcacaca |
| | | caattgatgacctcgtcgaaggaatgagttatgcaagcaaagtaagactcgatagtgccgagttta |
| | | aaaatcaaacttattacagtgccacattagtattagcgcagttgtctgcaagggctctatctataa |
| | | ataatgataaaatatatgcgctgagcttctttgaaagtagcctagaatccatccggaattataata |
| | | ataactcaaggcacataaacaaaatgatggataaaaatgatggtggctttagatatgcaatacaat |
| | | atcttaaggataatccattaatagaactccttcctcgtaaggacgaagttaatgaattaattaact |
| | | tctatgagagtcgtaagaaataatcatccttaaattaataaatggcaagtaactcattcccttgtc |
| | | atttattaaactcttaagagccttatcccgaaaagtattaatctgagctaataagattgtttttca |
| | | gctatgtcattattttattgccaatatatttacacttaagcattgacaggtagcggatagttattt |
| | | ttggcttgtaaataagccttttaataatagaactgtaagacaatcgctctgattttttgaaattta |
| | | tctcaatgttaaattcttccgcttttggcacaaacgggctagagcagacagatttaatgagataag |
| | | ggtatagatgaattctccatacccttgaacgattacttcccagttgatttgcttggtttcagtcct |
| | | ggggtattaccgggtgtatccttattatcacgtctgcgttgatcgggttttcctgttgattttgca |
| | | attggttttggaccaggtttaagccccataatcgtactccttagccatgtcagaggttattcctca |
| | | gtgtggatataaggggagcggtaagaattatcaagcttggatgggcggtgaaaaatgactacttga |
| | | ctattatgtgagcaatgtcagcttttgacatttagaggccagcccattactgaagtaagccaaaaa |
| | | tgagtcgcgatgagccctcaacaatgagggccacctcggagattg (SEQ ID NO: 288) |
| 41 | 17 | tattttgcgtagctagaacgcaatcaaatctagcagtccgctttgttcggagttcggacattatga |
| | | gttggcaagtaaagtagcttgctaggaagccggatttgcacggtcggtataataagatgtaacccc |
| | | ttgccttcatttactcgaatgaacgtgcacattggataggaggaaaaggaatgcaattcattacca |
| | | acggccctgatattcctgatgagcttttgcaggcgcacgaggaagggcgcgttgtgttcttctgtg |
| | | gagcaggcatttcctaccctgctggtttacctggtttcaaagggttggtagaactaatttaccaga |
| | | ggaacggaacaacactttcagaaattgagcgtgaggttttcgagcgtgggcaatttgacggcacat |
| | | tagatttgctggaacggcgcttaccagggcagcgtatagccgtccgacgcgcgttggaaaaagccc |
| | | ttaagccaaagctccgtcgtaggggcgctattgatactcaggcggcgctgttacgtttagcccgta |
| | | gccgcgagggtgcccttcgattggtcactaccaactttgaccgtctctttcatgtggcagctaaac |
| | | gtacaggccaggcttttcaggcctatgtagcgccgatgctgccaattccaaaaaacagccgctggg |
| | | atggacttgtatacctgcatgggctgttaccggaaaaggcggatgatactgccctgaatcgtctgg |
| | | ttgttaccagcggtgactttggcttggcttatctcactgagcgttgggcagctcgctttgtgagtg |
| | | agttatttcgtaactatgtggtctgcttcgttggctacagcatcaacgacccggtactgcgctaca |
| | | tgatggatgcgcttgcagcagatcggaggctcggtgaagtcacaccacaagtatgggcactggggg |
| | | agtgtgagccggggcaggagcaccggaaagccatcgagtgggaggccaaaggggtcactcctatcc |
| | | tttacaccgtaccggcgggctccactgatcattcagtgctgcatcaaacgttgcacgcttgggcag |
| | | atacttatcgagatggtatacagggcaaaaaggctatagtcgtcaaacatgctctggcccgcccgc |
| | | aggacagcactcgtcaggacgatttcgttggtcggatgttgtgggccttgtcagataaatcaggtt |
| | | taccagcaaaacgctttgcggaactcaatcctgcaccgccgctggattggttattgaaagctttct |
| | | cggacgaacgatttaaatacagcgatctgccacgcttttgtgtatctccgcatgtcgaaattgacc |
| | | cgaaactccgattcagtctggttcagcgtcctgcgccctatgagctggccccgcagatgtcgctgg |
| | | tttctggatgtgtcagtgctagcaaatgggatgacgtaatgtcccatatagcccgttggctagttc |
| | | gttatctgggcgaccctaggttgatcatatggattgctgaacgcggcggacaaatacacgaccgtt |
| | | ggatgtttctgattgagagcgaactagatcgcttagcagcactgatgcgggagcgtaagacttctg |
| | | agttagatgaaattctcttgcattcccccctggctattcctggtccacctatgtctactttatggc |
| | | ggcttctgcttagtggtcgtgtgaaatcgccattgcagaacctggatttgtatcgttggcaaaacc |
| | | gcttaaagaatgaaggcttgacgactacattgcgcttggagttacgcgggttgctttctcccaagg |
| | | ttatgttgaggcggccgtttcgctatagtgaagacgattcgagcagcactgatgaacccttgcgaa |
| | | tcaagcaattggtggattgggagctggtgctgactgctgattacgtacgttcaaccctgttcgacc |
| | | ttgctgacgagtcatggaaatcgtccttgccatacctgttggaagattttcagcagttgttgcgtg |
| | | atgcactggacttgttgcgggagttgggagagtccgacgatcgtcacgaccgctcgcattgggatt |
| | | tgccgtccatcactccgcactggcagaaccgggggttccgcgattgggtgagcctgattgaattac |
| | | ttcgggattcatggttagccgttcgagccaaagacagcgatcaggcctcgcgcattgctcagaatt |
| | | ggtttgagttgccatatcccaccttcaaacgtctggcactgtttgccgcaagccaagacaactgca |
| | | taccacctgagcggtgggttaattggttgttagaggacggttcatggtggttgtgggccacggata |
| | | ctcggcgagaggtattcagactgtttgttttgcagggacgacatctgacaggaattgcacaagagc |
| | | gtctggaaactgctatcttggcagggcctccgcgcgagatgtacgaggataatttggaagcagaca |
| | | ggtggcattatttggtggctcattccgtctggttgtgtctagcgaagctcaggggagcgggccttg |
| | | ttttgggagagtctgcggctacacgtttgacggaaatatccacagcatacccaaaatggcaactgg |
| | | caaccaacgagcgtgatgaattctctcactggatgagcggaaccggtgatccaggcttcgaggaga |
| | | gtatagatgtcgacattgcgccccgtaagtggcaggaattagtgcaatggctcgcaaagcctatgc |
| | | cagaaagactgcctttctatgaggacacttggagtgatgtttgccgtacgcgcttttttcacagtc |
| | | tgtatgcgttacgtaaactatcacaagatgatgtgtggcctgttggtcggtggcgtgaagctctgc |
| | | agacttgggctgaaccagggatgattttgcgttcgtggcggtacgccgcaccgttggtgcttgaca |
| | | tgcctgacgcagtacttcaggagatttcccacgctgtcacttggtggatggaggaggcttcgaaga |
| | | ccatcctctgccacgaggagattctactggccctttgtcgtcgggttctgatgatagaaacaagcc |
| | | cagagtctagcaccattcgaaacggaattgagacctatgatcctgtttctacggcgatcaatcatc |
| | | ccattgggcatgtcacgcaatcactgatcaccctatggttcaaacagaacccgaatgacaatgatt |
| | | tgcttcctgttgaattgaaaacacttttcaccaaattgtgtaatgtacagatagagctattccgcc |
| | | atggtcgggtgttgctggggtcgcggctgatcgcattttttcgcgtagatcgaccttggaccgaac |
| | | agtatctattgcccttgtttgcttggagtaatcccgtcgaagcaaaagctgtgtgggaaggcttcc |
| | | tctggtcgccacgcctgtatgaaccgttgctgatagctttcaagtcagattttttggagagcgcca |
| | | atcactattctgatcttggcgagcaccggcagcaattcgctattttcctgacttatgcagctctgg |
| | | gccctaccgagggatataccgtggaggagttccgaacggcaattagtgctcttccacaagaaggtc |
| | | tggaggtagccgcgcaggcgttataccaggcacttgaaggtgcgggcgatcagcgcgaggagtatt |
| | | ggaaaaatcgtgtccagccattttggcaacaggtttggccaaagtcccgcaacttggccaccccac |
| | | gcatatccgaatcgttgactcgtatggtgattgctgcccgaggtgaatttccggcggctttggcag |
| | | tggtgcaggactggctgcaaccgctcgaacaccttagctacgacgttcgccttttgctagaatcag |
| | | atatttgcagccgatatcctgcggacgctctatccctgctgaatgccgtgattgccgaacaacact |
| | | gggggcctcgagagttggggcaatgcttgcttcaaattgttcaagctgctccacaactggagcaag |
| | | atgttcgttatcagcgattaaatgaatattctcgaaggcgcagcgtgtgaaagtgacaggcgttgg |
| | | acagtgcgaactgtggagcctaacaaggtaaagacactctaactgataatgctgcgccgctcgtgc |
| | | aatgcaatacagtttttatctagcggtgaattatggtgttaaaagttagcccctgacacagggtgg |
| | | gtagttggctctgtgtcattgatgggtattagttctgatatgagctaataccca |
| | | (SEQ ID NO: 289) |
| 42 | 18 | gtaagacaagggttgagcaggctactaatcgttacacaggctaacaaaggcatattaagacgattt |
| | | gtagcgctgtaaccttgaaaattatgtacaagcgccccgcattacgtcgttttaaaggccatcgga |
| | | ttcaggcccgacgcggcttcacgcgattataaccgtgaaaaatcccccccgcatagaacctgaatt |
| | | atccccgccgccgcgcagaactgacagcgcttcagaaccgttaaccctctcagaaatcccgctttt |
| | | ttactgtaaaaaaccatgcataaggtgcatggttttgcatgcgtttcaccgacactgaatcccccg |
| | | ccagcgccagcagtagcgtgccctgaggccgttaatgcacccgtattaaaagcgccctgttaagcg |
| | | agcaggcggggcggggcgagcattgcgcgtcggtgttaccaattctatatggacattgagcaattc |
| | | aaatataataaaggttgggtatatttcgtcctcaacgatgtcaaaaactgcaaaagcgtattataa |
| | | ttcagatcattttcagaccacctattttaatcatgcatgcaaaatggaatatgtgatgacaaataa |
| | | aaacaaaatcaaaccattattaaataatatatccgctcgcctttgggatggtcgtgcagctatatt |
| | | gataggagctgggttcagtcggaatgcaaagccattaacaagcaaggcaagaaagtttccaatgtg |
| | | gaacgacttaggtgacattttttatgaaagtgtttactgcaaaaaaaacgacaatagatattcaaa |
| | | tgtattgaagctaggagatgaagttcaggctgcatttggtagagcgacacttgataaattaatcat |
| | | ggatcatgttccagataaagaatatgaaccatccaaattacatgtttcccttctttccttgccgtg |
| | | gattgatgtttttacgactaattatgatacattacttgagcgagcaagtgttaatgtcgactccag |
| | | aaaatatgacattgtccttaataaaaatgatttaatgaatgctgaaagaccaagaattataaaact |
| | | gcatggtagcttcccatcagaaaggcccttcatagttacggaggaagattacagaaagtatccttt |
| | | agaaaattctccttttgtgaataccgttcaacaatcattgattgagaatactctatgtctgatagg |
| | | attttcgggtgacgatcctaacttcttaaattggattggttggataagagataatcttggcacaga |
| | | aaattcacccaaaatatacttgatcggtcttttttcatttaatgaagcacaacgtaagcttttaga |
| | | aaaaagaaatatttccattgttgatttaagttttctaggtgattttggcaaggatcattatctagc |
| | | acaccaacgctttatccaattcttatacgaatcaaaaaatcgagacaacctaatagagtggccaat |
| | | agaaaccaattatgacagaattgtttttaatgatggcattgaattaaaaactgagaaaattaaaaa |
| | | gtgtatcttagaatgggctcagtcaagacaatcatacccgaactggcttattttgccggaatcaaa |
| | | cagaagtaatttatggcaaaacactatagattggttatctgttgctaattatgatgtcgcttggga |
| | | tggttctgatgatcttgattttggatatgaaattacatggcgactaaataaagctttgctaccaat |
| | | tttcaatgatacatcagaattcttatttaagttgattgaaaaatatgagatcaattacgtttcggg |
| | | gataaataataaaatcattgactttgatgaaaaatactctcatataaccctcagtttaatgagatt |
| | | ctgtcgacaagaaaaccttattgataaatggaagaatctaaacgatttattaattcaaaatcttga |
| | | tcgattaacaccagaggtaaaatctgattattattatgaaaatatattattttcatacttcaattt |
| | | aaacttcgatgaagccagaaacaaactctccaactgggaaacgaataaactcctcccccatcatga |
| | | aataaaaagagcaggattacttgccgaatttggaatgcttgatgaagcaatcaatcttcttgaaga |
| | | aactttatctacgattcgaagaaacagtttgctttcatctagaaacattgactattccagtgaatc |
| | | tcaagaagcatatggaatctatattttgcgaatgtttaaacggagtttgcgtttagatagcaaaga |
| | | tgacgattattcatctgagtataactcgcggttggctacattatcacaatatcgcagcgatcctga |
| | | aaacgaaataaaatacctagaaattaaactagagtcactaccaggtaccttcaagaataccaatga |
| | | cacggatttcgatcttaacaaaagaacggtgaccacttatttaggaggaagcccaacagaagtgag |
| | | gtcattagatgcttttagtttctttctactggcagaggaacttggcctccctttccacataccagg |
| | | aatgaacatttttagtggaatagttgagaatgcagctcgacatatttatcaatactctccagagtg |
| | | ggctattttttcaatatttagaacatttaacaaggataaggccaagagtctattcaatcgaaatag |
| | | aatttcgtctcttgagcgaaaaaaggttgaagatttatttgatggatactacaaaaaatatgagca |
| | | aattatcacaaaaaaaatagaagatagattaaacgataaacttgagatagaaatttctacgctatc |
| | | aatcattcctgaaattctttcccggctagttacaaaagtatcatttaataaaaagaaagacattat |
| | | tcaccttttgcttaaactgtttaactcggataattttcatcaatacatggagactaaagatctatt |
| | | aaagcgcactatttccaatttgagcgacttacaaaagatctcactaatagatattttcattgattt |
| | | cccctccgcgcctcccaatacccaattacatatgggtcaaagatacaacttccttactccatttga |
| | | atgtctattaggggttacaataacccccccaaaagaaaactctaaaaaaatcgcatctgcaaaatt |
| | | aaaaaaagatataaacgatttaaaaagtgataatttagacttgaggaaagctgtatcacaaaagct |
| | | cataacattatataacctagaaatgcttaacaaatctgacacgactaaacttataaaaaacctttg |
| | | gtcaaagcgtgataactttggattcccaataggcagtggttactataaatttttctttataaacaa |
| | | ccttaacccagataatgaaaatatagccgacaaattcatttctataattaaaacatacaaatttcc |
| | | tgtgcaagaaggaaaaagagttagtattacaggtgggttagatgagtattgtactgaactcaatgg |
| | | agcgctacaccatataagtcttccagagaaaaccctatctgaaataatttcaaaaatacatgactg |
| | | gtatgtcaaggatcgggcctggcttgaaaaaagagatgatttagccaaggagttcactcttagatt |
| | | cagaaatatcacaaatatcataacgacaattttagaacaccataaggacaaattacatgctgaatc |
| | | tataaatgaaatatcaagcctactagataaaatgaaagaagacaagatacctgtaaactcagcagt |
| | | aacaatgctttgtctgaaaaataaaagcacttacctcgagagaataaaagatatagagaatggact |
| | | atatagctttaataaagatgatgttattgaagctatcaactcaacttatgtctttattagaaacaa |
| | | tgaatttccactaaccatcattcaagctatcagcgataaaatcgcatgggatagaaaccctcgcct |
| | | tcctgattgctacaatttaattgcatatataattaactcgtgtgaatttactcttccagattattt |
| | | aatagagaaaatccttcgagggctggcatatcaaataaacattgatgatagagattttgttgataa |
| | | caatgaatatttgaatcaccttgagaaaaaacttagtgcaacaaagctggctgcttctatgtttag |
| | | aaaaaatgaaacactaggtattgaccaaccttctatcattcaagagtggaaaaacatgtgcaactc |
| | | tagaaatgagttcgatgaaattaggaatgaatggaacaacaatatataaataaaggaagaacaccc |
| | | aatttatattgggtgttctgttcacgaaacccttttaccataatcgaatggcaatataaattgaga |
| | | ttgaaatttattctcatctaattaatcagcccaccattg (SEQ ID NO: 290) |
| 43 | 19 | tagctattgtgactatgctaaccatatgaatctattgtgtgattatgagtaatgactttttctaat |
| 44 | | atttgatttttaatgtagtaacttagctaattttaaaatttgtaaaaggatgtttatgtcgattta |
| | | tcaaggtggtaacaagttaaatgaggatgattttcgttctcacgtttattccttgtgtcaattaga |
| | | taatgttggcgttctgttaggtgctggtgcttctgtcggttgtggtgggaaaacgatgaaagatgt |
| | | atggaaatcgtttaagcaaaactaccctgagcttttgggagcacttattgataaatatcttctggt |
| | | ttcgcaaattgattctgataacaatttggtcaatgttgaacttttgatagatgaagcaactaaatt |
| | | tctttctgtagctaaaactagacgatgtgaagatgaagaggaggaattcaggaaaatattaagttc |
| | | attatataaagaggttacgaaggctgcattattaacaggagaacagtttagagagaaaaatcaggg |
| | | taaaaaagatgcgtttaaatatcacaaagagttaatttcaaaattaatttcaaatagacagcccgg |
| | | tcagtcggctccggcaatttttacaacaaattatgatttggccttagagtgggctgcagaagattt |
| | | aggaatacagttgtttaatggtttttctgggctacatacacggcagttttatccccagaattttga |
| | | tttggctttcagaaatgtaaatgcgaagggcgaagcaagattcggacattatcatgcgtatctcta |
| | | taaattacatggctcacttacgtggtatcaaaatgatagcttgactgttaacgaagttagtgcatc |
| | | tcaagcatatgatgaatatattaatgacataatcaataaagatgacttttatcgcggtcaacattt |
| | | gatttatccaggggcgaataaatatagccatacaatcggcttcgtttatggagagatgtttagacg |
| | | ttttggggagtttatttcgaaacctcaaacagcgttgttcataaatgggtttggtttcggtgatta |
| | | tcatataaatagaataatattaggcgcgttactgaatccatctttccatgttgttatatattatcc |
| | | tgaattgaaagaagcaattaccaaagtaagtaagggtggtggttcggaagctgagaaagctattgt |
| | | tactttaaaaaatatggctttcaatcaagtaactgtagttgggggaggaagcaaggcatattttaa |
| | | tagtttcgtagaacatctaccataccctgtgctctttccacgagataatattgttgatgagttggt |
| | | tgaagcaattgctaatctttctaaaggagaaggtaatgtccctttttaaacttactgaaatctcgg |
| | | ctattggatacgttgtaggattagaaggggaaagaattaggataaacctgcatgaggggttgcaag |
| | | gcagattagcatcgcatagaaagggggtgagctcagtaacgcaaccaggagatcttattgggttcg |
| | | atgcaggtaatatattagttgtcgcaagagtgacagatatggcatttgttgaagcggataaagcgc |
| | | ataaggcaaatgtaggcacatctgatttagctgatatacctctaagacaaattatcgcctatgcaa |
| | | ttggctttgtgaaaagggagttaaatggttatgtttttatatcagaagattggcgcttacctgcat |
| | | tgggttcttctgctgttcctttgacttcagattttttgaacatcatttatagtattgataaagaag |
| | | aactcccaaaagcggttgaattaggtgtggattctagaactaaaaccgttaagatatttgcaagtg |
| | | ttgataaattattgtcgcgacacttagccgttcttggtagtacaggatatggtaaatcaaatttca |
| | | atgctttgttaacgaggaaggtttctgaaaaataccctaactcaagaatagttatttttgacataa |
| | | atggtgaatacgcgcaagcttttacaggtattccaaatgtaaagcacactattctaggggaatccc |
| | | caaatgttgatagtttggaaaaaaagcagcaaaagggtgagctatatagtgaagagtattattgtt |
| | | ataaaaagataccatatcaggcattaggttttgctgggttaattaaattattaagaccaagtgata |
| | | aaacacaattgcccgcattaagaaatgcattaagtgcaattaatcggactcattttaaaagccgta |
| | | atatttacttggaaaaagatgatggtgaaacttttcttttgtatgatgattgtcgtgacacaaatc |
| | | aaagtaaattggctgagtggttggatttattaaggcgtagacgtcttaaaagaacgaatgtatggc |
| | | caccgtttaaaagtttagcgactttggttgctgaatttggatgtgtagctgctgaccgttctaatg |
| | | gaagtaaacgtgacgcgtttggttttagtaacgtgttgccattggtaaaaatcatacaacaacttg |
| | | cagaggatataagatttaaatctattgttaatttaaatggagggggtgagctagcagatggtggaa |
| | | cgcattgggataaagctatgagtgatgaagttgattacttctttggtaaggaaaaaggacaagaaa |
| | | atgattggaatgttcatatagttaatatgaaaaatttggcacaagatcatgctccaatgttactta |
| | | gtgcattgttggagatgtttgctgagatactatttagacgtgggcaggaacgttcgtatcctacgg |
| | | tacttttgttggaagaagcgcatcattacctgcgtgacccttatgctgaaattgactcacagatta |
| | | aagcatatgaacgacttgctaaagaaggtaggaaattcaaatgctctttaattgtcagtactcagc |
| | | gaccctcagagctttctcctactgttttggcaatgtgttcaaactggttttcgttacgtttgacta |
| | | atgaaagagatttacaggctctcagatatgcaatggaaagcggtaatgaacaaatcttaaaacaaa |
| | | tatcaggtttaccaagaggtgatgctgttgcatttggttctgcatttaatttgcctgtaagaattt |
| | | caattaatcaagcaaggccagggccaaaatcttcagatgctgttttttctgaagaatgggctaatt |
| | | gtacagaattacgttgttaattacctgatgtacatggctagtgcaagttggtagcgcatgtctata |
| | | tgcatttatttgcatgtgttttattgagtgagcgcacaagcttgatgacccgacaggtatgtattt |
| | | agactgaa (SEQ ID NO: 291) |
| 45 | 20 | gtgcgccttatgtgattacaacgaaaataaaaaccatcacaccccatttaatatcagggaaccgga |
| 46 | | cataaccccatgagtgcaatagaaaatttcgacgcccatacgcccatgatgcagcagtattgaaaa |
| | | atataacatatccaactgattgtattgaaaatttaaaatagccatataacaaaaggttacacataa |
| | | gctactttttggggtttcaggcaagaaactaaaaattattaacgccatcaaattattcacatctta |
| | | ataattagcattgaaatttaatgtttttggttctttgtacatgtcaatggcttgtctttgtggcag |
| | | aatcataaagctatgcaatcattgcattgttattaacacagcatatttttatatacttttaacacc |
| | | ttacctcaaaaaggataacaaagtggacagaagtgcggttgatacaattcgtgggtattgttatca |
| | | ggttgataaaacgattattgagattttttcgttaccacaaatggatgactcgattgatatagagtg |
| | | cattgaagatgttgatgtctacaacgatgggcatttaactgcgatacaatgcaaatattatgaaag |
| | | taccgattataaccactccgttatatcaaagcccataagattaatgttgtcacactttaaggacaa |
| | | taaagaaaaaggggctaattattatctttatgggcattataaatccggtcaagaaaagttaacact |
| | | cccattaaaagttgactttttcaaatctaatttcctcacctacaccgaaaaaaaaatcaaacatga |
| | | ataccatattgaaaatgggcttaccgaagaggatctacaagcctttttggatcggttagttataaa |
| | | tatcaatgcaaaatcatttgatgatcaaaaaaaagaaactatacaaataataaaaaaccatttcca |
| | | atgtgaagattatgaggcagagcattatctttattctaatgctttcagaaaaacatatgatatctc |
| | | ttgtaataaaaaagatagaaggataaaaaaatctgattttgttgaaagtatcaacaaatcaaaagt |
| | | cttatttaacatatggttttatcaatatgaaggaagaaaagaatatttaagaaaattaaaagaatc |
| | | tttcatacgcagaagtgtaaacacctcaccttatgctcgttttttcatcttagaatttcaagacaa |
| | | aactgatataaaaacagttaaagactgtatatataaaatacaatcaaattggtctaatttatctaa |
| | | aagaacagatcgaccatattctccttttttactttttcatggcaccagcgatgccaatttatacga |
| | | attaaagaatcaattattcaatgaagatctaattttcactgatgggtacccttttaaaggaagtgt |
| | | atttacccccaagatgttaatcgaaggtttttcaaataaagaaatccacttccaatttatcaacga |
| | | catagatgatttcaatgaaacactgaacagtattaatataagaaaagaagtttaccagttttatac |
| | | ggaaaactgccttgatatcccatcccaactaccccaggtaaacatacaagttaaagactttgccga |
| | | cataaaggagatagtgtaatgagcaggaataatgatattaatgcagaagtagtatcggtatcgcca |
| | | aataaattaaaaatttccgtagacgatcttgaagaatttaagatagcagaagaaaaattaggtgta |
| | | ggatcttatttaagggtttcagataatcaagatgttgctcttctggcgatcatagataatttttct |
| | | attgaagttaaagaaagccaaaagcagaaatacatgatagaagcaagtccaataggtcttgttaaa |
| | | aatggaaaattctatcgcggtggagattcacttgcacttcctcctaaaaaagtggaaccagcgaaa |
| | | ttagacgaaataatatccatatactcagatagtatagatataaatgaccgttttactttttcaagc |
| | | ttatcgcttaataccaaagtatccgtacctgtgaatgggaatagatttttcaataaacatatcgct |
| | | atcgtaggttcaacgggttcaggtaaatcccacactgttgcaaaaatacttcaaaaagccgtagat |
| | | gaaaagcaagaaggttataagggattaaacaattctcatataattatttttgatatacattctgaa |
| | | tatgaaaatgcattccctaattcaaatgtattaaatgtagatacattaacccttccatattggcta |
| | | ttaaatggtgacgagttagaagagctttttcttgacacggaagcaaatgatcacaatcaaagaaat |
| | | gtgttccgtcaggcaataacattaaataaaaagatacattttcaaggagatccagccacaaaggaa |
| | | ataataagctttcactcgccatattatttcgacattaatgaagtcatcaattatattaacaataga |
| | | aataatgaaagaaaaaataaagataatgaacatatttggtcagatgaggaaggaaatttcaagttt |
| | | gacaatgaaaatgctcataggttattcaaagagaatgtaactcctgatggaagttcagccggtgct |
| | | ttaaatggaaaacttctcaattttgttgatcgattacaaagtaaaatatttgataagagattagat |
| | | tttattctgggtgaaggtagcaaatccgtaacatttaaagaaacattagaaactttaataagctat |
| | | ggaaaagataaatcaaacataacaatacttgatgtaagcggtgttccttttgaagtacttagcata |
| | | tgtgtatcattgatatctcgattaatttttgaatttggctatcattcaaaaaaaataaaaagaaaa |
| | | tctaatgaaaaccaagatatcccaatattaattgtttacgaagaagcacataaatatgctcccaaa |
| | | agtgatctgagcaaatacaggacatccaaagaagcaattgagaggattgcaaaagagggtagaaaa |
| | | tacggagtaacccttctccttgcaagtcagagaccttctgaaatttcagaaacaatattttctcag |
| | | tgtaatacttttatctcaatgcgattaactaacccagacgatcaaaattatgttaagcgattactc |
| | | ccggatacagtaggtgatattacaaacctcctaccatcgctcaaagaaggtgaggccttaatcatg |
| | | ggggattcaatatcaataccttcgattgtaaaaatagaaaaatgtacaatacccccatcgtcaatt |
| | | gacatcaaatatcttgatgaatggagaaaagaatgggtagattcggagtttgataagataattgaa |
| | | caatggagtaaaagttaatttcagaagtggattcactcttgctcaagagtgaatccactaatatca |
| | | tatcctaatgatatagtttaataaaatctattctggaatcattaggctgagag |
| | | (SEQ ID NO: 292) |
| 47 | 21 | accgtgctggcatgtttttacggagtgacgctttcattaacctgtacacgaacttctattccggca |
| | | tcatgacaggcctgcagccactgcgccacttccagcggatcgccctcccggcgtaccactctgcct |
| | | tctttattccataactgcagacaggtgctgccgtcgagacgcaccacaaaatccccacggcaggcc |
| | | tgataggggtttgagggccaaccgtacgaaaacgtacggtaagaggaaaattatcgtcttaaaaat |
| | | cgatttatgctatcacagtcgtctcttcaggtaagtacggttgcctttgcctgctttcttctcgtc |
| | | tggttaagttaagaaattcagagatccatgcttgagataaaagcggaataaaaccagtaaaatgta |
| | | actaaaacaacaacggaattgtatcaatgataatgtccacaccgtggctgacaccgatcgttgccg |
| | | atagtgatcatgctgaggcaaatgcagtgagctatgaagcactgactccgacagaactcgactcag |
| | | ataaagcaggctgttatatcagcgcgcttaattatgcttatgaacatccggatatccggaatattg |
| | | ctgttaccgggccgtatggggcagggaaaagctcagtattaaaaacatggtgcaaagctcacaatg |
| | | ggacactgcgggtgttaaccgtttctcttgctgattttgatatgcagagacatgtggatgaaagta |
| | | atggggacagcagtagtgacgaagggacgaaaaatactggtagtgttgaaaaatctattgaataca |
| | | gtattctgcaacaaatactctacaaaaataaaaagcatgagcttccctgttcccgcattgaccgta |
| | | tatcagatgtgactgcgggacaaatattgcggtctgcgtcttttctgacaggaaccattttactga |
| | | gtggagctgctttatttttccttgcgccggattacgttacaacaaagctatctttgccgggagcat |
| | | tcgcccgttaccttcttgaatgcccgtttggggtgcgtgtgtccggtgcagtggcatctgtgatgg |
| | | gatcgttatgcctgcttttgaaccagttacatcgtatcggtatatttgacaggaaagtaagtcttg |
| | | ataaagtggaccttctgaaaggcgctgttacaacccgggcatcatcaccttctttacttaatgtct |
| | | atattgatgaaattgtctatttttttgattcgactaaatatgatgtagtgatattcgaagatcttg |
| | | accgttttaacaatggccggattttcgtgaaattgcgggaaatcaatcaaattattaataactgcc |
| | | tttctgacagaaaacctgtaaaatttatttatgctgtcagagatggtattttcaactcagcagagt |
| | | caagaacgaaattctttgattttgttatgcctgttattccagtgatggataaccagaatgcttatg |
| | | agcattttgttaaaaaattcaaagaagaagagataaataataacttaagcgaatgtatttctcgta |
| | | ttgcgacatttattcccaatatgcgtgtaatgcataatattacaaatgagtttcgactctatcaga |
| | | atttagtcaatagtcgggaaaatctggccaaactacttgccatgatagcatataaaaatctctgtg |
| | | cggaagattatcatggtatagatagtaaaaaaggtgttctttatcattttattcaaagctacttag |
| | | accatgaaattcagaatgaattattacattctgcaaataacgaacttgaggatatggcacagtcac |
| | | ttgtagcgataacaaatgaaaaactcgcaaaccgggaaaatctgcgcgaagaactgctcatgcctt |
| | | accttagtaaaaattatagcggcgcgcttgttttttatacagaaggaaggcaaataagtcttgatg |
| | | atttgatacaagatgaagatgaatttctcatgcttttagataaggaaaatattcaggtcgttaccc |
| | | cctataacagacaaaattttctcatgataaatcagcgggatacagaaaaactgaagcagcagtatg |
| | | aaaaacgatgccatttaattgaaactaaatctgttgataatataaccagagtgaaaaataatattt |
| | | ccagtctggagtcattgaggaccgaaattctttccggaactgtagctgatatagcagaaaagatga |
| | | caaatgaaggctttgttgcctggataaagaagaaagaggatacaggtgtcctgacgattcagtcgg |
| | | aacatgaacagattgattttatattttttctgttatcaagtggttatttatcaacagattacatgt |
| | | cctatcgctcaatcttcattcccggagggctgagtgagacagataatttatttcttaaggatgtta |
| | | tgtctggtaaaggtccggaaaaaacattctcattccatcttgataacgttaataatattgttgaac |
| | | gactcaaaaagctgggggttctgcagcgtgacaatgctcaacatcctgctgttatcagatggctga |
| | | ttgataatgaccctgataccctgaaaaacaatataatggcattactgagtcagacgggtagccagc |
| | | gtgtggttagtttgctgatgttgatgcagaacgatttcacaacgtatgttcgcctgcgttacctgg |
| | | agatttttatgtcagatgaacatatactgaacagattgctggcacatttatgtgcgtcagaagaac |
| | | gcacacccgagcaaaagttttttgttcaggaaatagcggcacacctgttatgcctgactgaaaaat |
| | | caaatatctggcaatcggttgagattaataaacgtatcggtgagcttatagattcctccccaattc |
| | | ttattactgctgtgccaaaaggatatggtgatgcgttttttgaagtgttgaaagataatacacttt |
| | | cagtttcatatattccaggtgatgtgggagacgagaagtgttctgttatcaggaaaattgcgggtg |
| | | caggattattcaaatattccgtcagtaatcttaaaaatgtttatctttgcctgacgcaagacaaga |
| | | atgaagaaagaatgtcattctctctttatccgtttcattgtctcgagtccctggctatttctgaat |
| | | taacagaaattctgtggactaacatagaagattttattttatcggtatttattgaatcggaagaga |
| | | ttgatcgtattcctgaattgctgaattcttctgaagtctcaatgactgttgttgaacagattatag |
| | | ccaaaatggatttttgtataaataatctggatgatattattaatcgttcagagtgtgcggacaata |
| | | atgcttcagggagaaatatctatagcatgctgttgcagcatgacaggatttttccatcctttgata |
| | | atattattcatttattgcatgatacatcaattaatacttccggtgaacttgttcagtgggtaaatg |
| | | agaaacactttgaatttgaaccatctgatatagtcataaatgatacaggaatatttaataatttta |
| | | tttctgaattaatttgctcgccagtcatttcagaagaagctttactgaaagtactgagtaatttaa |
| | | acgttgttattatcgatgtgcctgaaaacattccattgcgaaatgctgaactgttatgttcagaga |
| | | aaaaactggcaccgacagttaatgtctttacggtgttgtttaatgctctcagtgaaaatgttgatg |
| | | atattaacaggatgaatactctgcttggtaaccttattgcccagcgtcctgagattattacccagg |
| | | agccagaagatattttttatatcgagggtgactttgatgaagaactggcaagcgaactttttcgtc |
| | | acaagctaatcggtatgaatataaaagttgccgctttacgctggttgcgtgataacaaaccgggaa |
| | | ttcttgataagagctacctgctgtcattagatattctggcagaactgagtccctggatgggtgacg |
| | | atgatctgcgcctgacactgcttaaacgttgtctggttgccggggatgctggcaaagacgcgcttt |
| | | gcgtggtgctgaacagttttgctgatgagagctatcatggactgttaccacatgacaggttcagga |
| | | aaatccctcactccgtggatttgtgggaagtggccgaattaatcagcaatcttggatttattcagc |
| | | cgccaaaaatggggtcagggcgtgatgaacacaaaattgttattactcccgtacgctatgtccgtg |
| | | atgttgagttttatgactgagcatcattgatacggtgttttaattgccttaaatacaaaaataaaa |
| | | acagattaatgcttaatgtgcattaatctgttttagttatcaatggctgttaattattgttaattt |
| | | tacattaatctttctttttcttcaggaagatccgaaaactcctggtcacggatcttcct |
| | | (SEQ ID NO: 293) |
| 48 | 22 | gaaattatttggaatggatgatggcgcttgattactggaacaggtctatgacatgaaggttatgat |
| 49 | | ttgttcactgctatgaggttaacactttaacaatttcccttactattcttgtactaattccttcca |
| 50 | | aatacttctgcttgagattaggatttatcctcttgtagtgttatttacaataaagattgtgatgct |
| 51 | | gatttaacccaacgtgttgtcagttgccttgctgaactaagttcagtatctagaaattagctcttg |
| | | atacatgagcgaatcagcgaaaattttcatcccgaccaattaatgaccgtaatggataggatgttg |
| | | ctgctatttggcttccatgagggaacatatgtttttaaacgatcaagaaacgtccactgacctgct |
| | | gtactacaccgctatcgccagcacagtggttaggcttgttgatgaaacgtcagatgcacccattac |
| | | gattggtgtgcatggtgattggggggcgggaaaatcaagcgtactaaaaatgcttgaggctgcctg |
| | | cgagaaaaaggataaaacgcactgtatctggtttaacggatggacgtttgagggattcgaagatgc |
| | | taaaactgtaatcatcgaaaccatcgtcgaggatcttgttgcctcgcgcccgatgagcaccaaggt |
| | | ggcagaagcagcaaaaaaggttcttcgtcgaattgactggttgaaaatggccaagaaagcgggggg |
| | | actggcgtttaccgcatttactggcatacccacatttgatcagattaaggggatgtacgaactggc |
| | | atccgactttctaagtgctccgcaggacaagctttctgctgcagatttcaaagcgtttgctgaaaa |
| | | agcaggaggcttcatcaaagaggccgatactgatagtaatacgctacccaaacatattcatgcttt |
| | | ccgtgaggagttcagggcgctgcttgatgctgctgaaattgaaaagctagtggtgatcgttgacga |
| | | tcttgatcgctgcctgcctaaaaccgcgattgaaacgctcgaagctattcgccttttcttgtttgt |
| | | agagaaaactgcatttgttatcggtgcagatgaagccatgatcgaatatgcggtaaaagaccattt |
| | | ccccgacctgcctcaaagcaccgggccggtaagttatgcacgcaactatcttgaaaagctcataca |
| | | ggttccatttcgaatccccgcactgggaactgcagaaacgcgtatatataccacgttgttgcttgc |
| | | agaaaatgcgttgggttcggaggacgacaattttaaagcattgctcaataaagcacgggaagagat |
| | | gaagcgtccttggatcagccgcgggcttgacagagaggcagtgatggcagcgttaaatggaaagat |
| | | tccggaggttgtggaaaacgcgctgctattcagcctacacgttacccctatgcttagttcggggac |
| | | acatggtaatccaaggcagattaaacgctttttgaactcaatgatgttacgccaggcgattgctga |
| | | tgaacgcgggttcggtagtgacattaagcgtcctgtactggcaaaaattatgcttgctgagcgttt |
| | | ttaccccagcgtatacggaaagcttgttcagcttgtatctaatcatccagagggaaaaccggaagc |
| | | tttggcggagtttgaagccttggtcagaggggggaaaactgctccgaagagtcgcgctgacagcaa |
| | | agagaattcctcagagtctgaagacgtccaaaactggctgaagattgattgggcgatcggttgggc |
| | | aaaagcagagcccgcactttctggagaggatcttcgtccatatgtgtttgtcactcgtgacaaaca |
| | | cagtactttgagtaatctggtcgtatcaagccatctcattcctataatggagaaacttcttggtcc |
| | | gaaaattgggatggtgaaaatcaaaggggatttagagaaactgagtccaccggatgctgatgaatt |
| | | attcgaaatgcttagcgataagcttttccaagaagacagtttcaatcgaaaaccaagaggatttga |
| | | cggcctcgaatatctcgtagaaacacaacctcaccttcaaaggagattgattgattttgcacggcg |
| | | cattcctgtaaaaaaagcagggggatggcttgctacccgtattgcgcaaagcctagtggaccctac |
| | | gttaatagaagaatatacaaaactgatccaagaatgggcgagtcaggacgaaaatctgtccctctc |
| | | taaatcagcaaaagcaaccctccagttatcgggatatcaacattaatgggaacctcaaaagcttac |
| | | ggggggcctgttcatggcctaatccccgatttcgtggagaatccatctccaccgaccctgccgcct |
| | | gttgaccctgcggatgatagcacgctggatacgccgctcattccaccggattcgagtggctcaggg |
| | | ccacttagcacaccgaaagcaaactttactcgatactcccgttcaggaagtcgtagttctctgggt |
| | | aaggcggtcgctggatatgtccgcaatggagtggggggcgcaggcagggccagccgccgtatgggg |
| | | gcctcacgcgctgcagcagggggactgctcggtctcatcagcgactatcagcagggaggtgctact |
| | | caggctcttgagcgcttcaatcttggtaatttggcagggcagtctgcatcgactgctcttctctcc |
| | | cttgttgaatttttatgccctccaggtggttctgttgacgagggggttgcgcggcaggctatgcta |
| | | gagaccatcgccgatatgtctgatgtaggagaggagaattttgatgagctcactcccgatcaatta |
| | | aaagaagtctttattggtttcgtggttcactccattgaagggaggctcatggcggatattggtaaa |
| | | aatgggatcaagttaccagacgacatagacgctatcgtcagtatccaggaggacctgcatgatttt |
| | | gttgatggagctactcgtacacagctccgtgaggagctgaggaatcttacagggctttcaggggat |
| | | gctatagacagaaaagtggaggagatttacaccgtggcatttgaattacttgcccgagaaggggag |
| | | agattggaatgagccatcataccttagttgcccgtttgggcactgacgataactccgatttacagc |
| | | tcagccgccaaagcacgcatctgacagaaattaattttctcaaagagaacggtaaactggatttcg |
| | | gtctcgggcaggcgctgaatggtttgagtgatcttggtttaacgccaatggatgtctccgtggatc |
| | | tggcactactggccgcaacggtgactgcggcggacacccgaatctcacgtgggcataacgctcaag |
| | | atctgtggacgcgcgaaattgcactttatatcccggtagcttccccgacattatggaatagtcaga |
| | | ctggattgctcagcaggatgttgaattttcttaccggcgaccgttggacaattcatttccgctcgc |
| | | gccctgttattgagcacgggctcattcagcgatcctctaaggaacgttcggtgaaccctacttctg |
| | | tttgcttgttttccggggggctcgacagcttcatcggtgccattgatttattatctaatgggggaa |
| | | ccccccttctgatcagccactactgggatacgactaccagcgtttatcagcagaagtgtgctcagc |
| | | tgctgtcggagcgatatggacaatcgttcagccatgtgcgagctcgtgttgggtttgaaaaaacaa |
| | | cgattgagggagaagatggagaaaacacccttcgtggccgctctttcatgtttttctcgctcgcga |
| | | caatggccgcagacgccctcggcgggccggtcacgataaacgtccctgaaaatggtttgatctctc |
| | | tcaacgttcccctcgatccgcttcgtgtcggagcgctaagtactcggacaacccatccgttttaca |
| | | tggcgcgttttaatgagctgctgggcaaccttggcatcagtgcacatctggaaaatccctacgcct |
| | | acaaaaccaaaggtgagatggctatccattgccatgaccatgcttttctaaggcaacacgcggctg |
| | | acaccatgtcatgttcgtctccgcaaagtacgcgttggaaccctgcgctgaatgagcagcaatcaa |
| | | cacactgtggccgatgtgttccatgcttaatcaggcgagcatcattgtttacagctttcggcacgg |
| | | acgatacgatttaccgtatcccggatctccgtagccgggtactggacagctctaagcctgaaggtg |
| | | aacacgttcgggcatttcaatttgctctggcaagattggcgcgatcaccgagtcgagcaaaatttg |
| | | atattcacaaaccagggccgctcagcgactatcccgactgcttagctgagtatgaaggtgtttatc |
| | | tgagaggaatgaaagaagttgaacgcctgctgagtggagtcataacgaggccccttacatgaaatt |
| | | agcaggacagaagcccgctccacaatgggtcgattttcactgtcatctggatctataccccaatca |
| | | ctctgcactcatccgtgaatgtgacatttcacgtgttgccacgctagcggtgacgacaacccccaa |
| | | ggcatggatgcgtaaccgggagttaacttccgattctccttatgttcgtgtcgcacttggtctaca |
| | | tccccagctgattgcggaacgtgagcatgagatagcgttactggagcactatctcccttctgcacg |
| | | ttacgttggggagatagggcttgatgccagcccgcgcttttatcgcagctttgaagcacaggagcg |
| | | gattttttcccgtattctgaatgcctgtttcgagcagggggataagattctcagcatccacagcgt |
| | | tcgcgctgcagccaaagtgttgggacatttggaaaacaccagacttactgaaaattgcaaggctgt |
| | | cctacactggttcactgggagtatctccgaggctcgacgagctgttgaacttggatgctatttctc |
| | | tattaatgaagagatgctacgttctcctaaacatcgaaagctggtgtcctttttgcctttcgaacg |
| | | tatcttgacggagaccgatggaccttttgtgtttcacgaagaaaaagcgatacaccctcgtgatgt |
| | | gcagcgtacggttcatgaaatcgcgcagatccaccacgtatcggacacagatgctgctatgagaat |
| | | actttataatcttcgaagtttagtcaccaatagttctcacagtgagaatagttcatgaatctaatt |
| | | agttggattaatacaggggaatagttgaatacttcagtcccctaaaagctaatatgctctatgtca |
| | | tctaatgataagtggctccaaagagccacttatcattaacttttctaaagggaggtagaagt |
| | | (SEQ ID NO: 294) |
| 52 | 23 | cggattgaatctgtttatgaaatttggctgctatcaactaatgggcgttaagttgattgtatgatc |
| | | tgattgataaagaaggggagactaaaaatctcctcttctttgcagcagtttactgcggtctttttg |
| | | tgatgcatcagcataaaacgttttacttgtggaccctaagaaatggagaacattatgtcgactgta |
| | | gatacctctacagcagaggaactcaatcaaggaggctcagattttattctgacttccctcgaggct |
| | | atgcgtaagaagttattggaccttacgtctcgaaatcgacttttgaatttccctatcactcaaaaa |
| | | gggtcttcactacgtattgttgatgaattaccagaacagctttatgaaaccctttgctcggaaatc |
| | | ccgatggaatttgctcctgtgcccgatccaactagagcgcagctgttagagcatggctatctcaaa |
| | | gttgggccagatggtaaagatatacagttaagagctcatcctagcgctaaggattgggcgcacgtc |
| | | ttaggaatccgtacagattttgatttaccagatagccataaaacggttgtttctgattcagataga |
| | | gagttgctggaaaaagcccatcagtttatcttgcaatatgcccaaggccagaatggaaaattaaca |
| | | gggattcgttctgaatacgttaatcaaggtatagctttgtcagcgttgaaggaggcgtgctgctta |
| | | gcaggctatgaagggcttgaggattttgaacgacaggcaaaggctgggaatgagattagtatatct |
| | | tcttccaatccctctcatgacgataatcggatacaggctctgctttatccaaatgaactggaagct |
| | | tgtttgcgcgccatctatggtaaggctcaaactgctttggaggagagtggcgccaacatcttgtat |
| | | ttggcgttagggttccttgagtggtatgaaagcgattcctctgaaaaggcacgttatgcaccgtta |
| | | tttacaattccggtgagatgtgaacgaggaaaattagatccgaaggatggtctttacaagtttcaa |
| | | ctttattacacgggtgaagatattttgcccaatctctctttgaaggaaaaacttcaggctgacttt |
| | | ggcctcgctcttcctttgttcaatgaagaggaaactccagagtcttattttgcttcggtgaagaag |
| | | gttgtagagcagcacaaacctaaatggtctgtgaaacgttatggtgcacttagcttgctcaatttt |
| | | ggcaagatgatgatgtatcttgacctcgatcctgcccgctggccttgtgacaagcgcaatatattg |
| | | tctcatgaagtaattcgtcgctttttcaccagtcagagctgtggtcaagagaattccggcttacct |
| | | ggtggcttcggtcagcatgagtactgcatcgatagttaccctgatattcatgacaaggttccacta |
| | | atcgatgatgcggatagctcgcagcacagtgcgttgatcgatgctatccgtggtcaaaacttagtc |
| | | attgagggccctcctggtagtggcaaatcacaaacgatcaccaacttgattgcagcagctctgctc |
| | | aacggtaagaaagtcctgtttgtggcagagaagatggctgcactggaggttgtcaaacgtcgcttg |
| | | gatcgtgcggggctaggtcaattttgcttagagttgcacagtcataaaactcataagcgcaaggtg |
| | | ctggatgatattaatgctcgcttggtgagtcaggcgaccatgcctactatggaagagattgatgct |
| | | cagattttgcgttatgaagatcttaagcagcagctcaatgaatatgccgcattgatcaataaccaa |
| | | tgggcgcaaacaggcaaaacgatccatcagattttgagtggtgcaacccgttatcgtcacaaatta |
| | | gatattgatgcaacagcacttcatatcgaaaacctttccgggaagcagttggataaagtgacccaa |
| | | ttacggctgcgtgaccaaatagtagaatttagccgcatctacaaagaggttcgtgagcaggtgggg |
| | | gctaatgcagaaatatatgagcacccttggagcggtgtgaataacacacaaattcaattgtttgac |
| | | agcgctcgtatagtcgatttgctacaaacttggcagacatcaattatcgactttcaacatagctat |
| | | caagaatatgtagataagtgggcgttagaaggcgaaagccttaatacgcttcaatatattgagcaa |
| | | ttggtagaagatcagtcgaatcttccagtgttgtgtggttcagagcatttcccagcacttagtgag |
| | | ctagattcacccgatgccattgcacgggtgcgtcactatttagataggttcgagttgctacaaggt |
| | | cattatgtggccttgagccaggttatcgagcctcaaaagctacgacttttagaacaaggacaatcg |
| | | tgtgactttcctcgtgaagagctggaaaaatatggtgcagcagaggatttcactttacgtgatttg |
| | | gtcaggtggcttgaatccatccaatcaattcatgatgagttatcatctatttatgcgcaattaaac |
| | | gatttcaaaaatgctttgccagatggtattgcttcgtatatcgatgattcgcaagctggattgcta |
| | | ttctgctctgagttgttgtcgattctgggtgctttaccgactgagcttattagagttcgagatcct |
| | | ctttttgatgatgatgatatcgatgcagtattgcgcgacttaatgtgtcaaatcgaaacattgcgt |
| | | cctttaagagatggtctatctactttgtatcaattggaccagttgccttcccaagagatgctcgcg |
| | | catgccgttgctgttatccagcaagggggattatttgcatggtttaagagtgattggcgtagtgcc |
| | | aaggcactgctcatggcgcaatctcgaaagcctgacactaagtttgctgagttaaaacgctgctca |
| | | gctgatttgctcaagtattcggagctgttacaacggtttgaacaaagtgactttggtaatcaactt |
| | | ggtaatgcattccgagggttggacaccgactgtgaacaactcatgttattgcgtgattggtacaag |
| | | aaggtccgagcttgttacgggataggttttggaaagcgagttgcgataggctctggattatttaac |
| | | ctagatggtgagattatcaaaggtgtgcatttaatcgagaaatcgcagattagctcaagattaatg |
| | | actttggttaaacgggtcgagcacgaggctaagttattaccgcgtatttctagcttgttggaagaa |
| | | catgcatcttggttaggtgagcaaggtgtattgatgcaatcttaccgacaggtgcggaatactctc |
| | | attgccttgcagggatggtttatcaatccagatatatcattagagcagatgactcattcctccgag |
| | | attttgcaaaacataaacgatcttcagatatcccttgaaaatgactcgttacagttaggggcgttt |
| | | ttacaattaaccccattggcttgcggtgcgtataaaaataatcaactgacgttagacactattaac |
| | | gacacgctgaattttgccgagcaactggttgataagataaattgcgtatccttggctacccagatc |
| | | agacatttggctagtggtagtgattacgatttactatgtcgtgatggtggagaaatagtttcgaaa |
| | | tggaatgaacagattaaaaatgctgagttatatgcgctagaaacaaagttagagcggagtcagtgg |
| | | ctcaagtcgactgatggttctcttaatacattaatcgagcgcaacgaaagagcaatacagcaaccc |
| | | cgttggttgaacgggtgggttaactttattcgttgttacgagcagatgcatgaaaatggattgcag |
| | | cgaatctggagtgctgtacttgcgggctcgctcccgattgaaaaagttgaattgggtttagcatta |
| | | gcaattcatgaccagctggcgcgggaggttattcacatccaccctgaattgatgagagtttccggc |
| | | tcacagcgcaatgctttgcagaagtcatttaaagagtacgacaaaaaactgattgaattacaacgt |
| | | cagcggattgcagcaaaaattgcttgccgaaatataccagaagggaattctggtggtaagaaaagt |
| | | gaatatacagaactagctttgatcaaaaatgagttgggtaaaaaaaccagacatattccaattagg |
| | | caattggttaaccgtgcatgtaatgcgctggttgcaattaaaccttgtttcatgatggggccaatg |
| | | tcagcagctcattacctagaacctggacgaatggaatttgatctggtggtgatggacgaagcgtct |
| | | caggtgaagccagaggatgcattgggtgtcatcgcgaggggcaagcaactagtggtcgttggtgac |
| | | ccgaaacagctaccaccaaccagtttctttgatcgaagtgccgacggagaagatgacgatgatgcc |
| | | gcggctttaagtgatactgacagcattttggatgctgctttgccactgtttcctatgagacgtttg |
| | | cgttggcactatcgttcacgacatgaaaagttgattgcatactctaaccgccatttttataacagt |
| | | gatttggtgatattcccttccccaaatgctgagtctccagagtatgggattaaatttacctatgtg |
| | | tcaaaaggtcggttctccaatcaacacaatattgaagaagcccaagcagttgctgaggccgtactt |
| | | catcatgcgcatcaccggccgggtgagtcactcggggtagtggccatgagttccaagcaacgcgat |
| | | caaattgagcgcgctatcgatgaattgcgccgaaatcgccctgaatttaacgatgcaatcgatggc |
| | | ttacatgccatggaagagccactttttgtgaaaaaccttgagaacgttcaaggggatgagcgtgat |
| | | gtaatctttatttcctttacctatggaccttctgagcatggtggaaaggtttatcaacgctttgga |
| | | cctatcaattccgatgttggctggcgtcgcttgaatgtgcttttcactcgatcaaaaaaacggatg |
| | | catgtgtttagttcaatgcgttctgaagatgtattgacgagtgaaaccagtaaacttggtgttatt |
| | | tcgttgaaaggttttttacagtttgccgaaagtggcaaactagattccctcacaacgcataccggc |
| | | agggctccagatagtgactttgaggttgctgtaatggaagcactcaatcacgctgggtttgagtgt |
| | | gaacctcaggtaggggttgcaggattctttattgatctagctgtgaaagatccaggttgtcctggc |
| | | cgttatttaatgggcatagagtgtgatggtgcggcttatcactcagctaaatctgctcgtgatcgt |
| | | gaccgtttgcgtcaagaggttctggagcgtttgggttggagaattagccgcatttggtccactgat |
| | | tggttcagtaatcctgatgaggttctatctccgattatccgtaaactccatgagcttaaaacattg |
| | | gctccagacgttgttgtaccttcctatgaatatgtcgaaacgattgagtcaagcgctgaagtggcg |
| | | tctgactcaattgattctcttatgcccaatttggggcttaaggagcaacttaagtattttgccaca |
| | | catgtcattgaggttgagcttcctaatgttgatgctgatcgtcgtttgttgcggcccgcaatgctt |
| | | gaggctttgctggaacatcagcctttatcacgttccgagtttgttgaacgaatacctcattatctg |
| | | cggcaagcaacagatgtatacgaagcacaacgctttcttgaccgagtcttggcattaattgatggc |
| | | gcagaggctgaagcgaatgatgcagcgtttgagtctgaattggcataattagttaaaggtaataag |
| | | aacagtgacaactgtcgg(SEQ ID NO: 295) |
| 53 | 24 | atgatgaagatcacctaaaatgataggttgtttttatacagtaccaaattcaattttctctctata |
| 54 | | agatagattgcatttccgcggatgtagtttacaagggaaagacggtcaacatgcatcgcactattt |
| 55 | | ctgagttttatcgcattccccctttacttattcgggcgctaaaaagtggaatttcctccgtggtgg |
| 56 | | agtttcatctcaacaggggattacccaaagattcacgagattctctgggaaacagcccattgatga |
| 57 | | ttgcggcccagtatggacatttcgctatttgcgaaatgttgttgagtgcgggtgttgatgttgaac |
| | | atcaaaacaacctcgggcttcgcgctagtgaccttgcgcaggagcaaaaattgcgtgatctgttgg |
| | | cccgttatcgtcagcctctttcacttgccgaactggaacgctctgtggtttcagtcgaggactcag |
| | | aaacagaggcagaattacccagcgctgaaatcccgatggattttatgctgtgggatgcagaagttg |
| | | aattgaagcccgccgaagataatctgacgttaagacatgcttccgctgaagcccagcaattattat |
| | | cacgctatcgcccgaaagataactctgctgagtggagcgatatcgaactcacgctccctgaaccac |
| | | tgacgccagtttctcactctccgcaaaattaccctcatctctcaacgttgctcattggcgcactgg |
| | | atacggggcgtatctctttgcgtgacatctggcatgccggggaagaggatttcggtatgcagtggc |
| | | ctgaattccggctcagcgtagaggcattgatcagggacttaccgctgattgtggatgacgatgata |
| | | ttattccgcctgacgctgctccggcgacattatcggtgagtgaacctcttgaaccctggtttgatg |
| | | ctttcaatgcattgcggcagttcggcatcgttgaaaactatctcgtggatatccgccagtgggatg |
| | | tcgtggataaaacaaaagaagaacgactcggccagcgcatggatacggcgctaattaatctgataa |
| | | gaatcctggcgggtttatccgaagcggaatatatgcagttgctgcagcccaattaccttccggagc |
| | | cagcgcctgagatttctgaagaggaagacgtcgcagaagaagcggatgaggaaatgcctcccgtat |
| | | ccgatgacgatgacgataacgatgacactatcagctttatcgagcttcttgttctgctgagaagtg |
| | | ggaaagcaggcgagtatcaggataatcatatcccccgcccggagtatgccgacctgcaacagatag |
| | | ttgagcgcgcccgaacgcttatccctgatgaaggtcataaaataagtctgtatgtcagcagttaca |
| | | gagaggcttgggaggggctgatccacgccaacttgcgtctggtcgtcaccatcgcgaataaatatc |
| | | gcgggcggggattagatgtcgaggacctgatccaggaaggtaatctgggtttgatcaaggccgttg |
| | | aaaaattcgactatcgacgcggatttaaattctccacgtatgccacctggtggatccgccagaaga |
| | | tcagccgcgcgattgccgatcaggcgcagctcatccgtttacccgttcacttctatgagcaattca |
| | | ggcgctggcgaaacagtcgggatcaattgctgtatcgccaggggataacgcccacgatcaaacggc |
| | | tgcaagcattgactgaccttccagaaaatcaactcaagcggatggcaaaatatgaagaacagacgg |
| | | tgttgattggcgattttcatgatgacgcccaggacagcgaagcggcgctgtcgggagacgcgatcc |
| | | tgaccggaaaggatttcaccagtgctcccgttcagtctctcgagctaagagaatgtgtttcattgg |
| | | tgctggaaacgttgttgccacgcgaaaaacagatcataaaaatgcgttttggcatcggtatgacgc |
| | | aagatttcacgctggaagaggtgggtaaacagtttgatgtcacgcgagaaaggatacgtcagatag |
| | | aagccaaagcgctccgtaagctccgctatcacagccgggcgtcgaaattaggcggcttcgtcgaac |
| | | agtgggaaaccgcgttgagcgagatgcaggaagaagaagaatgacgaccatgcgccatgcgccacc |
| | | gaatgcagccattatgatcgaagcgctgcgagggctcggttacaacactgccaccgcactggctga |
| | | catcatcgacaacagcattagtgccggtgcccgtaaggtcgatctgacctttcactggcgtgagtc |
| | | ggatagctatatcgtggttcgggataatggttgcggcatgtcggccgctgaactggatgttgcgat |
| | | gcggctgggggtcaaaaacccgctgacaaagcgttcaggacacgatctgggccgcttcggtctggg |
| | | actcaaaaccgcctccttttcgcaatgtcgccgtctgacggtcgcctccaaaaaagaggagataac |
| | | gaccatcctgcggtgggatctggacattctcgccgccagtacggacgacggttggtatttgcttga |
| | | aggcgctgacccaggaagtcaggaggcgttagcaaatgaggaacctgactcccacggtacggtggt |
| | | gctgtgggacgttttagaccgaattgtcacccccggctacggtgagaaagatttcctcaatctgat |
| | | ggatggcgttgaacaacatctggcgatggtatttcaccgattccttgaggggaacgctccccgact |
| | | cactctcaccctcaatggtcgcaaaattaaagcttgggatccctttctcagcgggcatccttccaa |
| | | gccctggcattcgccttcggcaatggcgccaggcgctcctgccgtgaaggtggagtgtcatgttct |
| | | gccgcatcaggatcacctgacgacgcaggagtatcaacaggctcaaggaccggcaggctggacggc |
| | | ccagcaaggattttatgtataccggaatgagcgattgctggtggcgggcaactggcttggactcgg |
| | | aagcccccgggcctggacgaaagatgaaacccaccgccttgcgcgaatccgtctggatatccctaa |
| | | tgatgccgacatagactggaagattgatattcgtaagtcgatggcccgcccaccggtttcgctgcg |
| | | gccttggttaacccaactggcgcaatcaacgcgtgatcgtgcggtacggacatttgcaaaacgcgg |
| | | gaaaatgaataagcgcaagcccggcgaggaacttgttcagctctggcaagcgcagaagacgccatc |
| | | cggtgttcgttatcagatttcgttacaacatcctgttatcagcaatgtcctttcgcaggccggtga |
| | | gttatctccacaaattcaggccatgctaagactgattgaggaaaccgttccagttcagcaaatctg |
| | | gcttgatacggctgagacaaaagagacgccgcggacaggttttgaaactgcaccgcccgcagaggt |
| | | gttgtccgtattgcaggtgatgtaccagactatggttggacagcaggcgatgtcaccggcgctggc |
| | | gaaacagcacctgcaaaatatggaacccttcgataattatcccgaattaattgcactactccccga |
| | | cgatcaacatgagaaatcgctatgagtcttaatcccttggatgacacgcaactgagtgtattgcag |
| | | attgtgcaaacgttcctgcaaagtcaggataaaagcacgatcacgcccggtattctgcgccaacat |
| | | attgatatggtttgtcagatgaaacctgagtggagccgccttgatagtcgggagatcctggtcgaa |
| | | gagttgatccgccgttacagcatctggatgggagaagattcttctctgagtaatgacgaagggcat |
| | | caaccctggctgaccgctgatgcgaaacgcgagtggcgctactggcatcgatatcgccagtggctt |
| | | ggcaaaacgatgccttggggagtcctggatacccttgaccgttcaacggatcgtgttctgggatta |
| | | cttgagcaaccggggcgggaagggcgttgggaccgacgtgggctggtggtcggccatgttcagtcg |
| | | gggaagaccagccactataccggtctaatctgtaaagccgcggatgcgggatataagataatcatt |
| | | gtgctcgctggtttgcataacaacctccgctcgcagacccaaatgcgtcttgatgaaggatttctt |
| | | ggttacgagacgagcccactcagagaaaaagtgaccatcattggggtgggcgctattgatagcgat |
| | | cctgtcattcgtcccaactacgtcactaaccgatctgaaaagggcgacttcagcgccggagtggct |
| | | aagaatctggggatcagccccgagcaacggccctggctgttcgtagtaaagaaaaataagtctatt |
| | | ttgaagcgcctgcatacctggattgagaaccatgttgccaccagcgttgaccccatcaccggaaag |
| | | cgttttgtttcggaattaccgctgctgatgattgatgatgaagcggataacgcctcagttgatact |
| | | ggggaaatcgtctacgatgacgatggaaaaccggatgctgaacatcagccaacggcaataaatagt |
| | | ctgattcgtaagctgttgatgcagtttagccgtaaggcgtatgtcggatataccgctacgcccttt |
| | | gccaatatttttattcacgagagcaatgaaacacgtgacgaaggtccggatttgttcccttccgcc |
| | | tttatcattaatctcggcgcaccctctaactacatcggccctgcgagggtatttgggcgggccacc |
| | | gcggaaggccggagcggagagtttcctttgattaggcgagtgagtgatcactgtagcgatgacgga |
| | | aaaagggggtggatgccggtttctcataagagttcgcactatcccacactggatacgctaactcat |
| | | ttcccggactcgttaaaacacgctatcgacagttttttactagcatgctgtgtcagagaattacgc |
| | | ggtcagggagagaaacacagttcgatgctggtccatgtgactcgcttcaataaggtgcaatcggtt |
| | | gtttatgaaaatattgatgcctacattcaggacgtgaggcagcgactgacgcgaaggattggacac |
| | | gaaccttttttacatcagcttgagtcactctggcaggccgattttttgccgacgaatcaggcgatc |
| | | cgcgaagttatgccgcagcaggttccggacgacgccttcgaatggcaggagatcgtcgacaagctg |
| | | tataccgtgatagaaaacgtgtcggtacgaatgataaacggaacggcgaaggatgcgcttgattat |
| | | tcggacagtgcgacaggcttaaaagtcattgcgattggcggagacaaactggctcgagggctaacg |
| | | cttgagggattatgcactagttattttttacgcgcctcccgcatgtatgacacgttaatgcagatg |
| | | gggcgttggtttggttatcgccagggatatctggatgtatgccggctttataccaccgatgagctg |
| | | attgaatggtttgagcacattgcggatgcgtcagaagagctgcgggaagagtttgacaatatggtc |
| | | gccagcggcggcaccccacgtgatttcgggctaaaagtgaaatcacaccctgtgttaatggtgacc |
| | | tcgcccttaaaaatgcgtagcgcgcgttcactatggctctctttcagcggcacagtggtcgaaacg |
| | | atttcgttgtttaaagaacaggagtatcacaagcgtaactacgtggctttccagcgtctaaccggg |
| | | cgcgtcggtgctggcgcgccgatacctgagagacgacgcggagataagattgaaaaatggaatggg |
| | | gtcatttggcaaaatatctcccctgagccgatcatcgatttcttaacggaatatgagacccatgct |
| | | caggccagaaaagctaacagcaaactactggcggattttgttacgcggatgaatcgcgttgatgaa |
| | | ctcacccaatggacggtggcggtgatagggggtggcatcgatcgccatcacgatgtttgcggcttt |
| | | tccgtaccgcttatgatgcgtaaagcgtctgaaggggtcactgaccgttattccattggccgttta |
| | | ctttccccacgcgatgaagggattgactgtgatgaatcaacttggcttgctgcgctggaagaaacg |
| | | cagcgtatttttcatgccgatcccggacgcaatgaagggcgagaggagcccgtcgttccaggtggc |
| | | gtggtactgcgtcggattaaaggatttggcattaacgacattccagcacagcgtcaaaaaggttta |
| | | ttgctcatttacttactggacccgcagcaggcattgtcggcagcggaatatcaggaagatgcctta |
| | | cctgtggtggcttttggcatcagttttccgggaagccgcagtggggtaacggtggagtacaaagtg |
| | | aacaacgtactatgggagcaagagtatggtgcggctgagtaaagacgatctgctggcggcctggaa |
| | | agccttagatcgatctcagatagacgaactgcctggcgctcagggctggcgcgggattcggctttt |
| | | tacgcaccagggctgtagctttcatgccgggcgtcgtcagcctgataatgaagaaatgctgattgc |
| | | cgtgtttcctcatcctctttcgcctgggtcggcggcgctgccatcttgtaaaggattccgcgttga |
| | | gatggccggaacagaggagggggggcagaacggtttgatgatccgtcgccagcaaacagggaatgt |
| | | ggatgtctttacgacgatgattctggatattctccattcgctcctgaacgtttcgaaaccgcgcct |
| | | gtttgaaactctgcttcgtcggattcgtttatggcaggcgtttatggagcgcgatacccgtccact |
| | | cagtcaagaagaagaagttgggttaatcggcgaattgacgtgtctggagcggttgatcgagagcgg |
| | | tcttgctccgtcaacggcagtcgaagcatggataggaccgcagcatgggctacaggattttgcact |
| | | cgatgaacgcgccattgagataaaaagcactacggcagcgaagggtttttgcataactatccactc |
| | | tcttgaacaactggactggcagcgggcaggatcgcttgtattgtgtggtttgcgcttcagcgagca |
| | | tcccaccggcgcaaccctgaatgacatcattagccgtcttcgtcaacggtttgagggaaacgctac |
| | | ggcggcttgtatttttgagggatcactttgtcatgtcggatatttcactgaacatgctgaattcta |
| | | tacacgtcatttcttgctgacagaggcgttcgcactccccattgaagcggattttccctctttgac |
| | | gcatgccaatgtcccgttgccggtggtgagtgcgcgctatcaactcgaactccagacacttattcc |
| | | tcaggcccaagattttaaccattgcttgtcagactttgcaggattaccgcatggaaattattgatt |
| | | ttttacgtcaaacccagaatgagattcgcaaggaatatcaggatcaaatggctcagccaggggttg |
| | | agtcgccttttccggagctgatttttaccgatattgttatgcgtcatatggccgatatcggcatga |
| | | cattcgatgatgccgagacgtgtcactttatggcgaaagtcagtggacacaatgtgcgtctcagcg |
| | | gttatgccttctcagaagatggcgatcaacttgacctttttgtcagtatttatcacggtagcgacg |
| | | agctctgtcacgtcccggatgctgagacaaaagcgattgccggccactgcattcaatttttgcaga |
| | | agtgcgttgacggtaaattatcatccacgctcgatcagtccaatgatgcctggcaactggtgacga |
| | | ccatcgaacagtcctatgcggaactggagcaaatcagaatttatgtactgaccgatggtcaggtga |
| | | aaacccgctggtatcagtcacgggacgtggccggtaaaaccattaaattagaggttatggacattg |
| | | tccgtttgtttaaccactggcaggaaggtaagccacgcgatgaactgcaggttaattttgatgagg |
| | | tggctgggggggcgcttccctgtgtctggatcccggatgaaatgggtgagtacgattatgcgctga |
| | | cggtggttccgggagagacactgcgatttatctatgaaaaatatggcaaccggattctggaagcga |
| | | acgttcgctcgtttctgagtcagacggggaaagtcaacaaggggattcgtgacactttacgtgagc |
| | | agcctgagcgttttatggcttataacaacggcattgtgattgttgccgatcaggtcaggcttggtg |
| | | aagcaccgggaggtggccctggtattgcgtggatgcaggggatgcagatcgtcaacggtgggcaga |
| | | cgacggcttccatgtttttcaccaaaaagaaatttccggcaaccaatctgcgtaacgtgcgtgtac |
| | | ccgcaaaagtaattgtgctgaaacagacgaataatgcacaagaagagatgttaattgcggatattt |
| | | cgcgcttctcaaatagccagaataaagtcaatatttccgatctgtcagccaatcgaccagtacatg |
| | | tacagctggaaaaaatggcaaacacggtgtattgcccggacggatacagtcgttggttttacgagc |
| | | gagcaaatggcagttataaggttatgctggaacgagaaggtaaaacaccggcgggcattaaacggt |
| | | taaaagacgcaattcctccatcccgtcggataacgaaaacggatttcgcaaaatatcactgtgcct |
| | | ggctccagcgtccggatttagtcagcctcggtgggcagaaaaactttgccgcattaatgacgatga |
| | | ttgacaaggatactgagcgttatggggatgaactgaacattgaaacttttaaaaattacattgcac |
| | | aggctattatttataaaaaagcctataagttgattaattcacttttccccgcatttaaggcgaata |
| | | tcgccgcctatactgttgccgcctattcacatctttatggtaacaaaacggatctggcagagatct |
| | | ggaatcaacagggtatcgaggaaactatggggaatcgtcttgtcagcttggctcaccgagtaaata |
| | | gccttctgactgaatcggcaaatggcaggatgatttctgaatgggcgaaaaagccggagtgctggg |
| | | actacgtgcgcagtaaaatctatttctccgcacagggaaaaaaggatgacttctcgcatggtgaaa |
| | | ttgcatgatgagttcagtatcaacatgatatgtgagtattactgacgtatggcagcggttgttttg |
| | | tatggatgtgctatggcatcgcatcaatatacaattaacagctg (SEQ ID NO: 296 |
| 58 | 25 | cgtgatgaatgaagcggctaaatacattaatgataattataatttaattcattaaaatcagtaata |
| 59 | | tataaatataaaagttgtgaaatgtgatattcgtcaaagcatgtcaaaaagttttgactgttcttt |
| 60 | | aggcatcattcgcaattgtctaacaacttgataggataggaacaatctcaaaaaggaaaatgacat |
| | | atggcatacgaagctcaaatcagccgtactaatccagcagcatttcttttcgtcgtcgatcagtca |
| | | ggttcaatgtccgacaaaatgtcttccggccgaagcaaggctgagtttgtcgccgatgctcttaat |
| | | cgaactttaatgaacctaatcactcgctgcactaagtctgaaggcgtacgtgattatttcgaaatt |
| | | ggtgttttgggttatggcggtcaaggggtttctaatggtttctctggttcactgggaggacaagtc |
| | | ctcaatccaatttctgctctcgaacagaatccagccagagtagaagatcgcaaacggaagatggat |
| | | gatggagctggcggaatcatcgagacagcaattaagtttccagtatggttcgatcctattgctagt |
| | | ggcggcacgcctatgcgtgaagccctgaccagagccgccgaagagttggtgacttggtgtgatgcc |
| | | catccggattgctatcctccgactatcctgcatgtgactgacggcgaatcaaacgacggtgacccg |
| | | gaagagattgccaatcatctacgacaaattcgcaccaatgacggtgaagttctgattcttaatatc |
| | | catgtcagttctctcggaaatgatccaatcagattcccctcctcagacactggcttaccggatgcc |
| | | tacgctaaactgcttttccgtatgtccagccctcttccggaacatctggtgcgtttcgcgcaggaa |
| | | aaaggtcatacggtcggtatagaatctcgtggattcatgttcaacgctgaggctgccgaactcgtc |
| | | gatttcttcgacatcggaacccgcgcttctcagttgcgttgattcagcaatgaaactggagttctt |
| | | agggacagttccgaaagatcctgaataccctaaggcgaatgaagataaatttgccttctccgaaga |
| | | tgggagaaggctggcgctatgtgatggcgcgagtgagtccttcaactcaaagttatgggccgatct |
| | | tcttgctcgtaaatttactgcagatccgaaagtaaatcctgaatgggtagcatctgctttagcgga |
| | | atattctgccacgcatgacttcccttctatgtcctggtcccagcaagcggcattcgaaagaggcag |
| | | ttttgcgacactaataggtgtagaggaatttgaagagcatcaggcggtagagattcttgctattgg |
| | | agatagcatcaccatgctggttgattgcgggaaactcatttgcgcatggcctttcgataatccaga |
| | | aaaatttaatgagcggccaacactgcttgctacgctgtacgctcataacaatttcgtcggtggaag |
| | | cactttctggacacggcatgggaaaactttttaccttgaaaaactcacccaacccaaactcctctg |
| | | tatgacagatgcgctcggcgaatgggcactgaaacaagcgctggcagaggattctggttttatcga |
| | | attactttcgctgcaaactgaagaagagcttgcagagttagttctgagagagcgtgcagcaaaacg |
| | | tatgcatatcgacgactcaacgctgcttgtactatcgttttaacgcggaaagtaaagatgccttac |
| | | ccatctcttgaacaatacaaccaagcgtttcagctacatagtaagctgctaatcgatcctgaattg |
| | | aaatctggtaccgttgccacgacagggttgggtctccccctagccatcagcggtggctttgcactg |
| | | acctatacaatcaaatcaggcgctaagaaatacgccgttcgttgctttcatagagagtcaaaagcc |
| | | ttagaacgccgttatgaggctatatccaggaagatttcaagccttcgctctccctactttctcgat |
| | | ttccagtttcagccccaaggggtcaaagtcgaaggaatatcataccctatcgtcaaaatggcatgg |
| | | gccaagggagagacgctaggagaattccttgaggtcaacaggcgttctgcacaagcaatagcgaaa |
| | | ctatctgcatcgattgaatcacttgccgcctaccttgaaaaagaaaaaattgcacatggtgatttc |
| | | cagactggaaacctgatggtctccgacggaggtgcaaccgtccagttaatcgactatgacggcatg |
| | | ttcgttgatgagattaagacattaggaagctcggagttggggcatgtcaattttcagcatccccgt |
| | | cgtaaagcaacgaatccgttcaatcacactctggatcgtttctcactaatttcactctggctggct |
| | | cttaaagccttgcaaatcgatccgtccatttgggataaatcaaattcggaactggatgcaatcatt |
| | | tttcgagctaatgactttgtagaccccggttcatcttccatcttagggatgctatcgggaattcaa |
| | | cagctttccacccatgtaaagaattttgccgcagtctgcgcttcagcgatggaaaaaacgccttcc |
| | | ctcggtgacttcattgcaagtaaaaacattcccatatcgctagcttcgatcagtatgaatggggat |
| | | attccagtcagcaggctgaaacccggttatatcggtgcctacaccgtcctgtcagccttggattac |
| | | agtgcttgccttcagcgagttggtgataaagttgaagttatcggaaagattattgacgtcaaactc |
| | | aataagacccgaaatggcaaaccatatatctttgttaatttcggagattggcgcggtaatatcttt |
| | | aaaatatcaatatggagtgaaggcattagcgctttaccttcaaaacccgatgcctcatggataggg |
| | | aaatggattagtgtaatcggccttatggaaccgccttacgttagcgggaaatacaaatattcacat |
| | | atctcaattacagtaacgactatcggtcaaatgaccgttctttcagaaccagatgcccgctggcgt |
| | | cttgctgggccaaacgaaagtcgacaaacattaacttctactagcagtaatcaggaagccttggag |
| | | cgcattaagagtaagagcaccacttcaactcctatgcccatgaacactaacgccacaactgcaaat |
| | | caggcaatccttaacaagttacgggcttctacgcaaactgtagcggcagcaagagcgcaaactcag |
| | | catgtagtacctaataaatcatcaacgcattatgtggcaccgacgggaacatcagcttcgcagcca |
| | | gttcaaaatattccgagccctgctagtacctcaaagcagcaaacctctcaaaaaaatatagttaca |
| | | aagattttgaaatggctttttggatgattggtacttgtaaagaacaagcgcaatttcagtggccgt |
| | | atcacttgcgcttgaggtgcctgcgggtatgatcttgcgacatacaccactaaaacgaattcgtgg |
| | | cggcacttttagcctgcccctgtgttttcccgaggatttac (SEQ ID NO: 297) |
| 61 | 26 | gggctgtttggttgaattaaaaatacgaactaaaaccaacaagagtcggaaaaaacttcaaaatgc |
| | | tgcttatggataatagtcatcttaaaaatgtacggaaaaagagactaaaatcagaaaaacatctgt |
| | | tatacattgacttaaagtcatcatctccgctatgagtcctcaatccaagttgacaaatgtttagcc |
| | | aggagttcccgtgaacgagcatctctctcatatggatgtacataccttgtttgaagaaatggacga |
| | | gcaggctgatggaataacgtttaaatactcatttgatgacatagcaaagagcaacgcattggttgt |
| | | cactgagtttgtcaattttgagcgtgacagcacggtagctttactcgccagccttcttactctccc |
| | | ggcacaccaatctcagtgtttgcgctttgagcttctgacgagccttgcactaattcactgcaaagg |
| | | tcagcagatagcaaatatcgatgacgtgaaacgctggtatgtcactattggggagtcgagtagtat |
| | | cgttggagaagatcctgctgaggacgtcttcgtcgcccttgttgataataaaaaaggtgattaccg |
| | | tgtgctagagggggtttgggaggcggcaggtttttatacacaattaatggtcgaaattgtatccga |
| | | catgccggatacgcaccgctatcgctcgctgaaacttgctatacaggcaattctccgtctctcaga |
| | | tgtcatttgtgctcgctctggcctttatcgttttcaggaaggcgcagacgaattccctgactctct |
| | | tgacaccgctggtcttgatgagaaaacgctctgttcaagggtaacgttgtccgagcgttctcttcg |
| | | agctgaggggatcaaacttgctgacttagcacctttcattcttgaaccttctcatataagtatgct |
| | | tggaaatcaggtccctggggagggaatgcttgaacaacggccattgctccgcacacgcgatggtat |
| | | tgtggttgtacttcctaccgccatgaccattgcacttcgccaggcagtgataacatttgcaaagcg |
| | | cacagaagaattgagcgagctagacaaagcgttagctaacgtctacagccttactttctccgagat |
| | | gccggtcttcggtaatggaggaaggttaagaagactgacatgggagaagtacaaaatgagccgaac |
| | | aacgatggtaacctccatcgtggatgctggtcatttgatggtacttcagttcgttttgccttccat |
| | | acagcaatatgccgataccggtttcaacaacttgctacagctagatgaagagaccacgcaatttct |
| | | agataactctgttgaacaaattacagttgacctcgccaaacaacccggctttcagcgtggcatcgt |
| | | cgtgcgcattgcatgtgggtggggggcgggttttatgggggtccctccccaactgccagatggttg |
| | | gggatttgaatggatgtctggtgcggactttgtccggttcggggcattacccgatatgtcaccaat |
| | | tgccttctggcgtgtgcaagacgcagtcgaaacgatcaggcaagctggtgttcgattaatcaatat |
| | | gagcggaactctcaatcttcttgggtggatacgtgccaatgatggccatatggttcctcatgacca |
| | | gttaccagatgaccgtatcacaccggaacacccgctaatgttaatgattcccacgaatttactccg |
| | | tggtatacgaatagcggcagacacaggatatgaccggcatcgcattagtgacaacaatggtaaatg |
| | | gcatcgagtgatgaggccttcggcagaagatttctttcccaccgagcgtcagagcaagtgctacgc |
| | | atcaattgatgatcttgaagcgcaacggctgacctgtgtatatgaggggcagggtaatctttgggt |
| | | aacgctcgaagctccagaaatggaagattggatgctcctcgttgagcttgccaaaatggttcgaac |
| | | atggattgggcggattggcgaggcactggaggtcttgagtgagcaaccaataaaaaaatcattaaa |
| | | ggtgtatctgcattttgatggtaacgacaatatcggcagatttgatggtgagaatttttctgatga |
| | | tatgaatacattttggcgacttgaacgaatccatgagcatggggcgattcgtgtggttcttcaaga |
| | | tgggtatcttgcaggttttcgtctaccggataaccgtgcagaacgagctctggtgcgcgcactcgg |
| | | tacggcgtttgccacacttcttcggatgaaagagccagtagacaaaggggtcactgttgagcagat |
| | | agcggtgcccaatgacagagcgcgcagcttccacataatgcaggcttatgacttcaaccaatattt |
| | | aggccgttcactaactaaacgtcttttagctattgaagatatcgactcagccgcagcccgaattga |
| | | gctagcatggcgtgctgtttcgacagatgcaccatcacgatatcagggtaaaaaggaagttggaaa |
| | | gctccttaatgatgtggttgatgtgctgatccaagacttactaagcgaactttcaagatttgaccg |
| | | taaacagacagtaatgcgattacttgaaaacgttgtaaaggcacgttgtgaagaggcgcactggcg |
| | | tagtactgcagcagcggtccttggcttgcatgcaggagaagagggtgtcgaagagacgatagctca |
| | | agaaatgagccgttatgcgggcgcagcgttaacttcccggctaatcattgaacttgccatctgtgt |
| | | gtgcccgacaagcggtggaattgaaccttctgatatggcactcagtaaacttcttgcacgggcatc |
| | | actgctttttcgcataggtggtatgtcagatgccgtacgtttcggtgctttgcctgctgatattcg |
| | | catctcccccttaggtgatctcctctttcgcgatgaactcggcaaaatggtgcttgaaccaatgct |
| | | ttcaaaagttactaacgaacggtttgaggaacaagcggcacaattcgagcaacactatgtgaaaac |
| | | tgccggaggggatgatgagaatagcaaacaagatagtgttgcggctgaaaccaccgaggaccaaac |
| | | cgatattttccttgcattctggaaagcagaaatgggcttcactctcgaggatggaatgcgatttat |
| | | ccagttccttgagtccatcggaatagagcaagaatcagcaatcttcgagatgcgaagaagccaatt |
| | | agcggatgctgctaaatcggctgggctcgcagatgaaactattgatgcgttcctcaaccagtttat |
| | | ccttagcgcgcgtccgaaatgggatgtagtgcccgatggatttgacctttctgatatatatccctg |
| | | gaggtttggccgacgcctttcagttgctgtacgtcccttgttacagattgaagagagtcacgatcc |
| | | actaattgttatcgcaccaggactcttgaatctgtcccttaaatacgttttcgatggcgcatacac |
| | | tgggcaatttaagcgtgacttctttcgcacagagggtatgagagacacttggttaggtggagcgcg |
| | | ggaaggacacacattcgaaaaaactttggagagagaacttcgtgaaataggctggacagttcgacg |
| | | tggcataggctttcctgaaattcttcgcaggaatctaccaggtgatccgggggatattgatcttct |
| | | tgcctggcgctcagaccgcaatcaagttctcgttatcgaatgtaaggacctctcacttgctcgtaa |
| | | ttactcagaagttgcctcgcaactatctgaatatcaaggtgatgacataaagggcaaaccagataa |
| | | actcaagaaacaccttaaacgcgtattactagccaaagaaaacatcgataattttgccaagttcac |
| | | ttcgatagcgaatcccgagattgtatcgtggctcgttttcagtggagcatctcccattgcctatgc |
| | | tcaatccaagattgaggctttggcaggaactaatgttggccgcccaagtgatcttctgaacttttg |
| | | atagatatgctgtgcgataagacgccctggcaactaagttaatcgttcctactactgatagtttta |
| | | aatcaagg (SEQ ID NO: 298) |
| 62 | 27 | gatggactggtactgtagattcaccgtggaccagcgaatctattatgtggtgagcagaacattaac |
| | | acatcaatgtaacgccgtaatcattgagtctttgccggggacgcttgacatctccgaaagaattat |
| | | atcgtgagtcttaaggggaatctcttgcttccggttatacatttaaccggatctagctataagact |
| | | gttacatctattgggattaggtcaggacagatagcctgaaagcttttatagtgagggacttcagaa |
| | | ataccctagaaaaggaactgttatggtaggttcgcgctggtataaatttgattttcataaccatac |
| | | tccggcttcgcatgattacaaaattcctgacatcagccccagagagtggcttctggcttatatgaa |
| | | acagcatgtcgattgtgttgtaatcagcgatcataacagcggagcctgggtcgacgtgttgaaggg |
| | | tgagctggagaatatgtcccgggacgccagcaccggcgacctgccggaatttcggccactgacact |
| | | ctttccgggggttgaactgacagcgaccggtaacgtacatattctggctgtgctgcacacgcacag |
| | | tacaagtgccgatgtggaaaggcttctggcccagtgcaataataatagccccattccgagtgaagt |
| | | ccctaaccatcagctcgttcttcaactgggccccgccggcatcatcagtaatatccgccgtaatcc |
| | | gaaggctgtttgtattcttgcgcacattgatgcagccaaaggtgtcttaagtctgactaatcaggc |
| | | agagctcaccgcagcctttcaggaaagtccccatgccgttgagattcgacaccgggtggaggatat |
| | | caccgacggaacccgccggcggctgattgataatttaccgtggctacggggctctgatgcgcacca |
| | | tcctgaacaagccggcgtgcgaacctgctggctgaaaatgtcatcccctgattttgacggactcag |
| | | gcatgcactgctcgatccggaaaactgtgtgctgtttgatcagctccctccggaggaacctgcgtc |
| | | atatttgcgcagcctgaaattcagaacccgccactgccatcctgtgggtcaggattcggcctcggt |
| | | ggaattcagcccgttctataacgctgtaatcggctcaagaggcagcgggaagtccacgctcattga |
| | | aagcattcgtcttgcaatgcgcaaaacagaaggtctcactgcgacccaggggagtaagctggacca |
| | | gttcattcggacggggatggaagcggattccttcatcgaatgtattttccacaaagaaggcacaga |
| | | tttccggctcagttggcgaccagacagtaagcatgaattacatatcttcagtgacggagaatggat |
| | | gcctgacagtcactggtcggctgaccgttttccactctcgatttacagccagaaaatgctctatga |
| | | gctggcttcggatactggtgcattcctgcgcgtctgtgatgagagcccggtggttaacaaacgggc |
| | | ctggaaagagcgctgggatcagctggaaagggaatatctgaatgaacaaatcacgttgcggggcct |
| | | gcgtgccagacagggaagtgcggattcgctgcggggggaattatcggatgctgaacgtgccgtcag |
| | | tcagctgcagtcaagcgcctattatccggtttgcagacagctggccctcgccagaaacgagctgtc |
| | | cgcagcaaccttacccctggagcactttgagcggcgtattgcagccattcaggctctggcagaaga |
| | | accgctgcagagatccgatatcccgccggaaccttccggtctgctgatggcatttatggcgcgcct |
| | | gtcatctgtgcaacagcagtatgaccagcggctcaatactctcctggcagaatatgctgcagagct |
| | | cgcgggtatcaggagagagcaatcttttattgccctccgaacagcagtgagtgaccaggaaacaaa |
| | | tgtagaaagtgaagctgtttccctgcgggccagagggcttaatcccgatgttctcaacgaactgat |
| | | ggcacgctgtgagtcactgaaaaatgagctgagaaattacgacggtcttgatggggcgatctctgc |
| | | ctctgttgcacggtctgagcagttgctggctgaaatgcgtgcccacagaatggcattgacagataa |
| | | ccggaaggcgtttctctcctccctgtcgctcagcgctctggaaatcaaaattcttcccctctgcgc |
| | | cccttatgaagatgttatatctggttaccagacggttaccggcatcagtaattttgccgaacgtat |
| | | ctacgataacagtgacgggagcggattactgagcgactttatcagtgaacgtccgttcagcccgtt |
| | | gcctgccgcaacagagaaaaaatacagggcgctggacgagctgaaagcgctgcatcacagcatccg |
| | | gctggataattcagaggctggggcggggcttcatggttctttccggaatcgtctcaggagtctgaa |
| | | tgaccagcagctggatgccctgcaatgctggtatcctgatgacggcatccacatacgttaccagac |
| | | ccccggggggcagatggaagacattgcctttgcttctccggggcaaaagggagcgagtatgctgca |
| | | gttcctcttatcctatggcaccgatcctctactactggatcaaccggaggatgacctggactgcct |
| | | gatgctgagcatgagcgtgatccctgccatcatgtcgaacaagaaacgccggcagctgattatcgt |
| | | gtcgcactctgcccctatagtggttaacggcgatgcagaatatgttatcagtatgcagcacgatcg |
| | | cacaggcctgtatccaggactctgcggtgcactgcaggaagctccgatgaaggcactgatatgccg |
| | | tcaaatggaggggggagaaaaagcgtttcgttcgcgctatgagcgtattcttagctgaagaacgga |
| | | accgtccttaaggcggccatgaccggagagtgggcctggcggctgaatgcctggataaaagacgca |
| | | aatgtcagactgatggcctctgcgtctttg (SEQ ID NO: 299) |
| 63 | 28 | atagaacgatgaaggatggaagctacatattctcggtactaagatttatttttctgacacaaaatg |
| 64 | | accatttggcgttacataatcccaaaaaaacgtatcaaaaatctcaaaatgcgttacgattagaga |
| | | gtattttgattctgcgtgctcattttttgattgctgtggctttttgttgtgggagtgttgaatgga |
| | | ttatttatcagaagtgttaaaaatcattgaaggtgcaacaaaggcaaatgcttcgatggctagtaa |
| | | ttatgctgggttgctggcagataagctcgaacaaaaaggggaggtcaagcaagccagaatgataag |
| | | agaaaggttgcttagagctccccaggcgttggcaggagctcaaagggctggaggtgggatatctct |
| | | gggctcattaccggtagatattgatagtcgactcaacactgttgatgtcagttatcctaaattaga |
| | | cagttcagagatttttctgcctgcagcaatcagtacccgtgttgaagagtttatcactaatgttca |
| | | acgttatgatgagtttgttaaagctgatgcagcattgccgagtcgtatgctcgtgtatggaaagcc |
| | | aggaacaggtaagactatgttatctaagtacatcgctacccgcttagattttccacttcttacagt |
| | | gcgttgcgatactttgattagtagtttattgggacaaaccagcaaaaatcttagacaggttttcga |
| | | ttatgtaatgcagaggccatcagtgctttttttagacgaatttgatgctttagctggagcaagagg |
| | | taatgagagagatataggtgagcttcagcgagttgtcatttcactattgcagaatatggatgcggc |
| | | atcagaggatacggtaattattgcctcaactaaccatgagcaacttctggatcctgcaatctggag |
| | | gcgatttagcttcagaattccaatgcctctgcctgacatacatcagagagagttaatttggaaaaa |
| | | tcgtttaaagaatatgatatgtagcgatctagatttaagtgatttatcaagaaaatcggaaggatt |
| | | atccggagcaataattgaacaggtgagcttggatgcacgtagggatgcagttattgaaggtgcaag |
| | | tgtgataaatcaccataaattgtataggcgtttgtatcttgctcaatcgcttatggaaggtgtaaa |
| | | tttaagcacttacgaagatgaaattcgttggttacgttctaaagataaaaaattattttctatcag |
| | | agttcttgctaatttgtacaaacttacatcaagagtaatttcaaacattctgaaggagtcaggagc |
| | | atatgagcagaaggggtacacagtttagtaacgcaaaagttacaaacccaatgttaagaatccctt |
| | | tttccagtagtgacttgggtgcaatagtaaacgctggcggtggggcaaaggtattggttgatgtaa |
| | | cagccgaatatagacaagggctagtaagaaatttaacaaccagtaaacattatttagaatccaaac |
| | | tttcagagtaccctggaagcttgggtactttggttttcaaattaagagaccagggaatagccaaaa |
| | | cgcataggccgaacaaaattgctcaagaggctggattgcaaaatgccggtcatgccaaaatagatg |
| | | aaatgttggttgctgctcatgccggctgttttgacgtattagagtcagtcattttacatcggaata |
| | | ttaaagcgattttggctaatctaagcgcgattgagcgcattgaaccttgggatgagaataggaagg |
| | | ttccaggaggcactgatggtttgtttgaatcatcaaacatccttgtacgactatttgagtacacag |
| | | gtgaagatgcaacttacaacaactatgaaaacgttatttctatattagaacaacacggagttaaat |
| | | atgatgagattagacaaaaatgtggtcttcccttattaaggataatggatttatccccaaatgata |
| | | gatatatattagacattctcattgattacccgggtataagaacgttaattcctgaaccaaaatatt |
| | | cagcattcccggttagtgtaagtgattctgttggcattgaaacaaatagctttcccgtaccatcag |
| | | aagaattacccattgttgctgtatttgacactggggtaagccccatcgcggcaacaattactcctt |
| | | gggtagtgagtagggaaacatacgtaattcctcctgatacgagttatgaacatgggactatggtgt |
| | | cttcattgatatcaggcgctcattttttaaatgacaatcatccatggattcctgatacaaaatcta |
| | | aaatccatgatgtttgtgccttagatgaaaatggatcttatatatcagatttaattctgaggctag |
| | | cagatgctgtaaataaaagaccagatataaaagtctggaatttgtctttgggaggcggaccatgta |
| | | atgagcagacgtttagtgattttgcgatggagttagatcggctcagcgataaatttggtattttgt |
| | | ttgtagttgctgcaggtaattatgtagatgaacctatacgtacatggccaaatcctgatccgcttg |
| | | gaggtgctgatttaatttcctctcctggagagtcagtccgagcactaacagttggttcagtttctc |
| | | atatggaagctaatgatgctttaagtgaaattggaacaccgacaccatatactcgtcgtggccctg |
| | | ggcctgtatttactccaaagccagatataatccatgctggcggtggggttcatagaccttggaatg |
| | | taggagcaagcagtttaaaggtcgtagggccagataataggctttgctctaattttggtactagtt |
| | | ttgctgctccaattgtggcaagtttagctgcgcatacatggcagagaatagccactaatacagact |
| | | ttaatgtttcaccatcattgattaaagcattattaattcattccgctcaattatcttctcctgatt |
| | | actcgccaagtgaaagacgctatttgggagcgggaattcctaatgaagttattgagaccttatatg |
| | | atagtgatgataggtttactctgattttccaaacattcttggttcctggggtgaggtggagaaagg |
| | | ataactatcccataccatcggcacttattcaaaatggaaaatttaaaggtgagattgtaattactg |
| | | ctgcatatgcaccaccactgaaccctaatgccggcagtgaatatgttcgcgcgaacgtagagctaa |
| | | gttttggcttaattgagaataatactataaaaggaaaagtgcctatggaaggagaaaacggtcaat |
| | | ctggatatgagagagctcaaattgagcatggtggaaagtggtcaccagtaaaaattcatcgcaagg |
| | | catttaataaaggaattacttcgggtaactgggctcttcaggctaaaacaacgttgagagcgaatg |
| | | aaccggccttaatggagcctttacctgtaactattgtagtaactttaaaatcattagatggaaaca |
| | | cacaagtttatgctgatggcgtaagagctttaaatgctaataactgggctcactatccattgcctg |
| | | ctcgtgtgccagtttccgtataacaactatataaatcaaacccgctgtagcgggtttgatttattt |
| | | gtgggtgtgttttataaaaataccgcccatacacaacaaaatacaa (SEQ ID NO: 300) |
| 65 | 29 | cgtgattcagttcgccagactgcagcgttttccatgaatataactccatctggtttagaaagagtt |
| 66 | | ccaatctaacgatattgggaccagaatcacaggcggcagtggctttacgcttacaataactattct |
| 67 | | atcctgacaattttaagcctcgtttgttacgatgtaaccctataactatgtggttcctcaaccttt |
| 68 | | tttgcccaaaaaatgcccaatgaagtccaaagtggaaaacagatggttatccgttgatgagattgc |
| | | agattacctcgcgattaagcgagacacggtatacaagtagatcgcaaagaaaggtatacctgcaca |
| | | catgattggacgcctttggaaatttaaaaaggatgaagtagatggctggatacgcgatggcaaagc |
| | | tggcgaaaacagtaatcaagaataaaaaagcaaatttaggagcagtttaatgaaaaccgtacgtag |
| | | tgcatgccagttgcaaccgaaggccttggaaatcaatgtcggcgaccagattgaacagcttgatca |
| | | aatcatcaacgacaccaatggccaagagtactttaaaaagaccttcatcactgacggttttaaaac |
| | | tttgctctccaagggtatggcacgcttagccggtaaatcaaacgatactgttttccacctgaagca |
| | | agctatgggtggtggtaaaacccacttgatggtcggctttggtttattagcaaaagatgctgccct |
| | | tcgaaatagccacttaggatcaatgccataccaatcagattttggctcagccaaaatagcagcatt |
| | | caatggacgcaataatcctcattcctatttctggggtgagatcgctcggcagctaggtcgagaggg |
| | | tgtattcagggagtactgggaatccggagccaaagctcccgatgaacaagcatggataaatatttt |
| | | tgatggtgaggaacccatcctaatcttgttggatgaaatgccaccatacttccactactacagcac |
| | | ccaagtccttgggcaaggaactatagctgatgtagtgacacgggctttttccaatatgttgaccgc |
| | | agcgcagaagaaaaagaatgtatgtattgtagtttccgatcttgaggcagcttacgatacaggagg |
| | | caaactgattcagcgtgcattggatgatgctacgcaagaactcggacgcgccgaggtatccattac |
| | | gccggtaaacctcgaatccaatgaaatctacgagattctgcgtaaacgtttgtttttgtctctgcc |
| | | agacaaaaatgaggtctctgaaattgcgtcgatctatgcatcaagacttgcggaagccgctaaagc |
| | | caaaaccgtagagcgcagtgcagaagcattggcaaatgacatcgaatctacttacccattccaccc |
| | | aagctttaaaagcatcgttgctttgttcaaagaaaacgaaaagttcaaacaaacccgtggtttgat |
| | | ggagttggtttctagactgcttaaatcggtgtgggaaagcgatgaagaggtgtatttgatcggtgc |
| | | ccaacactttgatctttcgatacacgatgttcgtgagaagctggctgaaatttcagaaatgcgcga |
| | | tgttatcgcaagagatctttgggactccaccgacagcgctcatgctcagatcattgacctcaataa |
| | | cggcaaccactatgcacaacaggttggtacgctattgctaacagccagcctctccaccgcagtgaa |
| | | ctcagttaagggcttaaccgagagcgaaatgctggaatgtttgattgatcctaaccatcagggtag |
| | | tgactaccgaaacgcattcactgaacttgctaaatcagcttggtatttgcatcaaacacaagaagg |
| | | gcgcaattacttcagtcaccaagaaaatctcaccaaaaagcttcagggatatgccgacaaagcacc |
| | | tcaaaataaggttgatgaattaattcgtcaccgactagaggaaatgtatagaccagtcacgaaaga |
| | | agcatacgaaaaagtactaccactccctgaaatggatgaagcacaggccacactgaggagtggtcg |
| | | tgccctgttaataatcagcccagatggcaaaacaccacctggtgtagtcggcaacttctttaaggg |
| | | cttggtaaacaaaaacaacattctggtattaacgggcgataaatcctctattgccagtatagaaaa |
| | | ggctgcacgccatgtttatgctgttaccaaggcagacaacgaaattacagcatcacatccgcagcg |
| | | caaagagttggatgagaagaaagcacagtatgagcaggacttccaaactacagtgctctctgtatt |
| | | cgataagctcctgttccccggtaacaatcgaggtgaagacgttttacggcctaaagcgctggatag |
| | | cacctatccatccaacgaaccatacaacggtgaacgccaagtcgtgaagactctcacgtccgaccc |
| | | catcaagctttacacccagattaacgaaaatttcgacgcactgagagcccgagcagagtcattgct |
| | | gttcggtactttggatgaggcaagaaagacagatttgctcgataagatgaagcaaaaaacacagat |
| | | gccttggttgccaagccgtggcttcgatcaactcgctatcgaggcataccagcgaggtgtatggga |
| | | ggatttaggcaatggctatattacgaaaaagcccaagccaaaaaccactgaggtaatcatcagcga |
| | | ggactcatcaccggatgatgccggcaccgttcgtcttaaaatcggcgtggctaatgcaggtaacag |
| | | cccacgcattcattatgctgaagatgacgaagttaccgaaagcagcccagtacttagtgataacac |
| | | gctagcaaccaaagcattgcgagtgcagtttttggcagtagaccctaccggtaaaaaccttactgg |
| | | aaacccaaccacctggaaaaatcgactgacattacgcaatcgctttgacgaagtggcgagaacagt |
| | | cgaattgttcgttgccccccgtggcacaatcaagtacaccctagatggttcagaagcacgtaatgg |
| | | tgaaacctacaccgtgccaatccagctcgctgatcaggaagccactatctatgtctttgctgaatg |
| | | tgatggcttagaagagaagcgaaatttcacctttgcggcagcaggttctaaagaaataccgatcat |
| | | aaaagataagcccgccactctggtcagcccctcacccaaacgtatggatagctcggcaaaaaccta |
| | | cgagggtttgaaaatcgccaaagagaaaggcattgagttcgagcagattagcttaatggttggatc |
| | | tgcaccaaaggtgattcatatatcgctaggtgagatgaaaatcagcgccgaattcattgaaaccgt |
| | | attaacgcacttgcaaaccgtgttaagtccagaagcccctgtggtcatgaccttcaaaaaagccta |
| | | cacacagactgggcatgatcttgagcaatttgttaagcagcttggcattgaaatcggtaatggcga |
| | | ggtggaacaacgatgaataaaaccgttgattttggggcaccgtcagaattcggtatgcatcacttc |
| | | tatgtggagattcccgcagcgccccgtgacgctgttgtgatctatgaagactatggctttgacggt |
| | | gaagattctcgccgagaaacagtagagtgtcgcctgatattagccagagagctctggactaagatc |
| | | cgcgatgacgttcgccgtgactttaacgctcgcctaaagattaagaaacaaagctccggtacttgg |
| | | tctaccggtaaagtgaagcttgaccgctttcttggacgtgagttgtgcgttcttggctgggcagca |
| | | gaacatgcctcacccgatgaatgtctggttatttgccaaaagtggctggctttacgcccagaagaa |
| | | agatggtggctttacagtaaaaccgcagctgaagcaggtcgtgatgatcaaacacaacgaggctgg |
| | | cgtaaagcgctctattgcgcgctatcggatggagccaatatcaaattggaaaccaaaaagaagccc |
| | | aagtctaaaaagctacaagttgaagatgagacccaggatctgtttgggtttatggaaaagggagag |
| | | ttttgatggccttgcaaccgtttgaatggagagacaaaccgtctcttattgagcacctgttcccgg |
| | | tacaaaaaatatctgccgagacctttaaagaacgaatggcaagccacggtcagttgctggtgtcgt |
| | | tgggtgctttttggaaaggcagaaaacctctcatcttaaacaaagcgtgcattctgggctcattgt |
| | | taccagcaactgacaacccgcttgaagatttagaggtatttgagctgttaatgggcatcgactctg |
| | | agtcaatgcaaaagagaattgaggcttcactaccagcatcaaaacaagaaacaatcggcgattact |
| | | tggtattaccctatgccgaacaaatcaggattgctaagcgcccggaagaaattgatgaatctcttt |
| | | tcgtccatatttggaatcgggtcaacaatcatcttggtacttctgctcacacttttgcgcaactag |
| | | ttgaggaactaggtgttgcacggtttggccataggccaagagtggcagatgtattttctggttcgg |
| | | gtcaaattccgtttgaggctgctcgcttaggttgcgatgtctatgcctctgacttaaacccgatct |
| | | cctgcatgcttacttggggcgctttgaacgttgttggtgcgagcgcgcaaaaaagagtagaaatag |
| | | acaaagcccaacgggatatcgttaagaaagttcaaaaagagattgatgagcttgacattgagtccg |
| | | atggccgaggatggcgagcaaaggtattcctatactgcgttgaggtgacctgccctgaatccggtt |
| | | ggcgtgtgcctttaattccaagtttgattatcagcaatagttttcgagttgttgctgagcttaagc |
| | | ccgttcctgctgagaggcgatatgatattagtatccgtgaagtatcgactgatgaggaactggagt |
| | | tctataaatcaggcaccatacaagatggcgaggtaattcactcgccagatggaaaaactcagtatc |
| | | gcgttaatatcaaaacaattcgcggtgactataaagaaggcaaggagaacctaaacaagctgcgaa |
| | | tgtgggagaaaacagactttgctcctcgtcctgacgatatttttcaggatagattattttgcgttc |
| | | aatggatgaaaaaaaaacctaaaggatcgcagtattactacgaatttcgtactgtaaccaatgacg |
| | | acttaaaacgcgaaaaaaaggtaatagaacatgtcgcatccaaattagatgactggcagaagcaag |
| | | gtcttgttcctgatatggttattgaagcgggcgataaaacggatgagccaatcaggacgcgaggct |
| | | ggactcattggcaccatttattccatccaaggcagttgctatttttgagcttggtgaacaaatatt |
| | | cactcgcagaaggaaaatttaacttcttgcagtgcatgaatcacttgtccaagctaactcgctggc |
| | | gaccccaggccggtggtggtggcggttctgcggctacatttgataatcaggcgctcaatactctgt |
| | | acaactacccagttagagcaacaggatctatcgaaaatatcttggctgctcagcacaaccactgtg |
| | | gaatcagcgagaatgtttcctttgtggttaattcacatccagcgccagagttagatgtggaaaacg |
| | | acatttatattactgatcccccatatggcgatgctgtcaagtatgaagaaatcacagagttcttta |
| | | ttgcctggctgaggaaaaatccgccgaaggaatttgcccactggacttgggatagtcgccgatctc |
| | | ttgcggtaaaaggagaagatgagggtttccgtacaggcatggttgctgcttatcgcaagatggcgc |
| | | agaagatgccagacaatggtttacaggtgctaatgtttacccatcaaagtggcgctatctgggcag |
| | | acatggctaatatcatttgggcgagcggccttcaagttactgccgcatggtacgtagttactgaaa |
| | | ctgactctgcattacgtggtggttctaacgtaaaaggcaccatcatcctcattttacgcaagcgcc |
| | | atcaggcattagagaccttccgcgatgatttaggttgggaaatcgaagaagccgttaaagagcaag |
| | | tcgaatcgttaatcggattggataagaaggttcgttcccaaggcgcggaaggcctctacaccgacg |
| | | ctgacctgcaaatggctggttacgcagccgcgttgaaagtactgacagcttattcccgtatcgacg |
| | | gtaaagacatggtgactgaagccgaggcaccacgccaaaaaggcaaaaaaacttttgttgatgagt |
| | | taattgatttcgccgtgcaaacggcagttcagtttttggtgccggttggcttcgagaaaagcgaat |
| | | ggcagaagcttcaagcggttgaacgcttctatctgaaaatggccgaaatggaacaccagggtgcaa |
| | | aaaccttggataactatcagaacttcgccaaggcgttcaaggttcaccattttgatcaattgatga |
| | | gtgatgcctcaaaggctaactctgctcggctaaagctttctaccgagttcagaagtaccatgatgt |
| | | caggtgatgccgaaatgactggcactcctctgcgagcccttctttatgccttatttgagatatcga |
| | | aagaagttgaagtagacgatgttcttttgcatctcatggaaaactgcccgaattacctgcccaata |
| | | agcaactgcttgccaaaatggcggattacctggctgaaaagcgtgaaggtctaaaaggtaccaaaa |
| | | cgttcaaccctgagcaggaagcaagcagcgcgcgtgtccttgcggaagccattcgaaaccagaggt |
| | | tgtaatctatggcgattaagcgcttttcatcccgcacagaaagattagatacggaattcctcgctg |
| | | aatcgttgaaaggggctgctaagtatttccggattgcgggttatttcaggagctccatctttgagc |
| | | ttgtaggcgaagagattgcaaagattccagaagttaagatcatctgtaattccgagcttgatctgg |
| | | ctgacttccaggtagctactggccggaatacagcactcaaagagcgctggaatgaagtggatgtag |
| | | aagctgaagcgctactgaaaaaggagcgctaccagattttggatcagctattacattcgggtaatg |
| | | ttgagattcgcgtagtccctagggagcggttattccttcacggcaaagcaggctcaattcattatg |
| | | cagatggcagccgtaaatcttttattggctcagtgaatgaatctaaaagcgcattcgctcacaatt |
| | | atgagcttgtttggcaagacgatgatgaagaaagtgcggactgggtagaaagagaattttgggcac |
| | | tctggactgaaggcgtcccgctgcctgatgcgatcttagctgaaatccaccgtgtatctaatcgcc |
| | | gggaagtaaccgttgatgtattgaaaccagaggaagtcccagcggcggccatggcagaagcaccta |
| | | tctaccgtggaggggagcagttacagccctggcaacgctcgtttgtgactatgtttctggaacata |
| | | gggagatctatggcaaggctcgcctactattggctgacgaggtgggtgttggtaaaacgctatcaa |
| | | tggcaaccagtgcattagtcagtgctttactagacgatggacctgttttgattctggcaccttcta |
| | | cactcacgattcagtggcaaattgagatgatggacaagctcggtgtgcctgctgcggtttggtcct |
| | | cgcagaagaaagtttggctgggtgtagaggggcaaatactctcacctcgaggtgatgcctcctcta |
| | | tcaaaaaatgcccttatcgaattgccattatctctaccggactgattatgcatcagcgggagaaga |
| | | ctgactttgttaaagaagctggaatgcttctgaagaatcgtttcggtaccgttattctggatgagg |
| | | cgcataaagcccgtattcgtggaggattaggagatcaagcttcagaacctaataatctcatggcct |
| | | tcatgctgcagatcggcaggcgtacacggcatctggtactgggtactgcgacacctattcaaacca |
| | | acgtacgtgagttatgggatttattgggtattttgaactctggtgctgaatttgtactaggcgatg |
| | | ctctgtcgccatggcatgaccatgaacaagcgattccgttgataaccggccagactcaggtgacat |
| | | ctgaggctgaagtttggcattggttaagcaaccccctgccgccaagcaatgagcaccatactgttc |
| | | agcaaattcgtgactacctgtccattgataataagtcctttggatattctcatcgtttcgaagatc |
| | | tcgactatatgattcagagtctttggctctccgaatgcatgacacctagcttctttaaagagaaca |
| | | accctatcctacgccatacagtgctgcgtaagcgtaaacagctggaagatgacggtctgttagagc |
| | | gtgaggggtgaatacacatcccattaagcgcaacctagctcagtatcagtcgcggtttgtggggct |
| | | tggcattccgaccaatacaccattccaggtcgcttacgaaaaagcggaagagttcagtaagttgct |
| | | tcagtcacgcactcgagccgcaggcttcatgaaatctttgatgttgcaacggatctgctcaagttt |
| | | cgcatcaggcttaaaaactgctcaaaagatgttgaaacatacggtttctgacgaagacgaggatct |
| | | agttgaagatgttgagcacttactttcagaaatgactcctgcggaggtcgcttgtttaagagagat |
| | | tgaaacacaactgtcacgccccgaagccgttgactcaaaactgaacacagtgaaatggttcttaac |
| | | ggaattccgtaccgatggaaaaacttggctggaacacggctgtattattttcagccagtattacga |
| | | cacggcggagtggatagcgaaagaactggccaagtccttaaaaggcgaagtggtagccgtttatgc |
| | | tggcgttggtaaaagcggcttattcaggggcgaacagtttaataacgttgaacgcgaattgattaa |
| | | atccgcagtgaagacgcgcgagattctattagtggttgctacggatgccgcctgtgaaggcttaaa |
| | | cctgcaaaccttgggaacactcatcaatgtcgaccttccctggaacccatctcgtttagagcagcg |
| | | cctcgggcgaatcaaacgttttggtcagacacgtaagtttgtggatatgctcaatcttgtgtacag |
| | | cgaaacacaagacgagaaagtttataacgtgctgtcggaacgcttacgcgatacatacgacatttt |
| | | cggcagccttcccgatacgattgatgatgaatggatcgacaacgaggaagaactcaacactcgcat |
| | | ggatgaatacatgcatgaacgaaagaaagctcaagatgcgttctccgttaagtatcgcggtactct |
| | | cgatcctgatgctcatctctgggaacgttgcgctacagtactgtcacgtagggacattgtaagtaa |
| | | gctcagcgaaccatggggaagctaattatgttgtgatgtggatgccccgctcagccaaggtcctgc |
| | | acaactatgttggatgctcttttttagagggctacatcatgaattcgatcaaagttattggtacaa |
| | | ttctgagtaaatctgtctctcagggtatccatttcgagtg (SEQ ID NO: 301) |

TABLE 15-C
Sequences of validated defense systems (Sequences encoded by the genes
corresponding to rows 1-68 of Table 15-A)

| Row | |
|---|---|
| No. | Sequence |
| 1 | MIKNDKAWIGDLLGGPLMSRESRVIAELLLTDPDEQTWQEQIVGHNILQASSPNTAKRYAATIRLRLNTLDKSAWTLIAEG |
| | SERERQQLLFVALMLHSPVVKDFLAEVVNDLRRQFKEKLPGNSWNEFVNSQVRLHPVLASYSDSSIAKMGNNLVKALAE |
| | AGYVDTPRRRNLQAVYLLPETQAVLQRLGQQDLISILEGKR* (SEQ ID NO: 302) |
| 2 | MIDPVLEYRLSQIQSRINEDRFLKNNGSGNEIGFWIFDYPAQCELQVREHLKYLLRHLEKDHKFACLNVFQIIIDMLNERGLF |
| | ERVCQQEVKVGTETLKKQLAGPLNQKKIADFIAKKVDLAAQDFVILTGMGNAWPLVRGHELMSALQDVMGFTPLLMFYP |
| | GTYSGYNLSPLTDTGSQNYYRAFRLVPDTGPAATLNPQ* (SEQ ID NO: 303) |
| 3 | MNIEQIFEKPLKRNINGVVKAEQTDDASAYIELDEYVITRELENHLRHFFESYVPATGPERIRMENKIGVWVSGFFGSGKSH |
| | FIKILSYLLSNRKVTHNGTERNAYSFFEDKIKDALFLADINKAVHYPTEVILFNIDSRANVDDKEDAILKVFLKVFNERIGYC |
| | ADFPHIAHLERELDKRGQYETFKAAFADINGSRWEDERDAYYFISDDMAQALSQATQQSLESSRQWVEQLDKNFPLDINN |
| | FCQWVKEWLDDNGKNILFMVDEVGQFIGKNTQMMLKLQTITENLGVICGGRAWVIVTSQADINAAIGGMSSRDGQDFSKI |
| | QGRFSTRLQLSSSNTSEVIQKRLLVKTDEAKAALAKVWQEKADILRNQLAFDTTTTTALRPFTSEEEFVDNYPFVPWHYQI |
| | LQKVFESIRTKGAAGKQLAMGERSQLEAFQTAAQQISAQGLDSLVPFWRFYAAIESFLEPAVSRTITQACQNGILDEFDGNL |
| | LKTLFLIRYVETLKSTLDNLVTLSIDRIDADKVELRRRVEKSLNTLERLMLIARVEDKYVFLTNEEKEIENEIRNVDVDFSAI |
| | NKKLASIIFDDILKSRKYRYPANKQDFDISRFLNGHPLDGAVLNDLVVKILTPKDPTYSFYNSDATCRPYTSEGDGCILIRLP |
| | EEGRTWSDIDLVVQTEKFLKDNAGQRPEQATLLSEKARENSNREKLLRVQLESLLAEADVWAIGERLPKKSSTPSNIVDEA |
| | CRYVIENTFGKLKMLRPFNGDISREIHALLTVENDTELDLGNLEESNPDAMREVETWISMNIEYNKPVYLRDILNHFARRPY |
| | GWPEDEVKLLVARLACKGKFSFSQQNNNVERKQAWELFNNSRRHSELRLHKVRRHDEAQVRKAAQTMADIAQQPFNER |
| | EEPALVEHIRQVFEEWKQELNVFRAKAEGGNNPGKNEIESGLRLLNAILNEKEDFALIEKVSSLKDELLDFSEDREDLVDFY |
| | RKQFATWQKLGAALNGSFKSNRSALEKDAAAVKALGELESIWQMPEPYKHLNRITPLIEQVQNVNHQLVEQHRQHALERI |
| | DARIEESRQRLLEAHATSELQNSVLLPMQKARKRAEVSQSIPEILAEQQETKALQMDADKKINLWIDELRKKQEAQLRAAN |
| | EAKRAADSEQTYVVVEKTVIQPVPKKTHLVNVASEMRNATGGEVLETTEQVEKALDTLRTTLLAVIKAGDRIRLQ* (SEQ |
| | ID NO: 304) |
| 4 | MNTNNIKKYAPQARNDFRDAVIQKLTTLGIAADKKGNLQIAEAETIGETVRYGQFDYPLSTLPRRERLVKRAREQGFEVLV |
| | EHCAYTWFNRLCAIRYMELHGYLEHGFRMLSHPETPTAFEVLDHVPEVAEALLPENKAQLVEMKLSGNQDEALYRELLL |
| | GQCHALHHAMPFLFEAVDDEAELLLPDNLTRTDSILRGLVDDIPEEDWEQVEVIGWLYQFYISEKKDAVIGKVVKSEDIPA |
| | ATQLFTPNWIVQYLVQNSVGRQWLQTYPDSPLKDKMEYYIEPAEQTPEVQAQLAAITPASIEPESIKVLDPACGSGHILIEA |
| | YNVLKNIYEERGYRGRDIPQLILENNIFGLDIDDRAAQLSGFALLMMARQDDRRIFTRDVRLNIVSLQESLHLDIAKLWQQL |
| | NFHQQVQTGSMGDMFAENNALTQTDSAEYQLLMRTLKRFVNAKTLGSLIQVPQEEEAELKVFLDALYRLEQEGDFQQKT |
| | AAKAFIPFIQQAWILAQRYDAVVANPPYMGGNYMETELKNFVSSYYPQGKADLYSSFMVRLLLQLKDNRTLSLMTPFTW |
| | MNLSSFEELRKIILTNFSIQSLVQPEYHSFFESAYVPICAFSISNTPLSWNAKFFDLSDFYGEKNQAPNFQYAIKNDNKCHWK |
| | YNRITTDFLCTPGYIIAYSLPDSALSCFKTSKKLHDVCNLKQGLITGDNERYLRFWHEISYNSFSLNEKRKKTKWFPYQKGG |
| | AYRKWYGNNDYVVDWENDGYSIKNFYNDKGKLRSRPQNIQFYCKEGLTWTSLTISSLSMRYVPNGYIFDAKGPMCFPKS |
| | SLDIWNILGYANSKVIDIFLKQLAPTMDYSQGPVGNVPFKFNDGDLNEIIKELVNIHKRDWDENETSFEFKRDMLVHFSRDI |
| | NTIKGSFTLRQGENKKAINRTKFLEEMNNSFFINCFNLTDILSPEIELNKITLTHATIEIDIQKIISYAIGCQMGRYSLDREGLVY |
| | AHEGNNGFADLVAEGAYKSFPADSDGILPLMDEEWFDDDVTSRVKEFIRTVWGEEYLRENLDFIAEVLKPKKGESALETIR |
| | RYLSTQFWKDHLKMYKKRPIYWLFSSGKEKAFECLVYLHRYNDATLSRMRTEYVVPLLARYQANIDRLNDQLDEASGGE |
| | STRLKRERDSLIKKFSELRSYDDRLRHYADMRISIDLDDGVKVNYGKFGDLLADVKAITGNAPEVI* (SEQ ID NO: 305) |
| 5 | MQNQDFIAGLKAKFAEHRIVFWHDPDKRFIEELEQLKLESVTLINMTHESQLAVKKRIEIDEPEQQFLLWFPHDAPPHEQD |
| | WLLDIRLYSSEFHADFAAITLNTLGIPQLGLREHIQRRKAFFSTKRTQALKNLATEQEDEASLDKKMIAVIAGAKTAKTEDIL |
| | FNLITQYVNQQIEDDSELENTQAMLKRHGLDSVLWEMLNHEMGYQAEEPSLENLLLKLFCTDLSAQADPQQRAWLEKNV |
| | LLTPSGRASALAFMVTWRADRRYKEAYDYCAQQMQAALHPEDHYRLSSPYDLHECETTLSIEQTIIHALVTQLLEESTTLD |
| | REAFKKLLSERQSKYWCQTQPEYYAIYDALRQAERLLNLRNRHIDGFHYQDSATFWKAYCEELFRFDQAYRLFNEYALLV |
| | HSKGAMILKSLDDYIEALYSNWYLAELSRNWNEVLEAENRMQAWQIPGVPRQQNFFNEVVKPQFQNPQIKRVFVIISDAL |
| | RYEVAEELGNQINTEKRFTAELRSQLGVLPSYTQLGMAALLPHEQLCYQPGNGDIVYADGLSTSGIPNRDTILKNYKGMAI |
| | KSKDLLELKNQEGRDLIRDYEVVYIWHNTIDATGDTASTEDKTFEACRTAVAELKDLVTKVINRLHGTRIFVTADHGFLFQ |
| | QQALSVQDKTTLQIKPENTIKNHKRFIIGHQLPADDFCWKGKVADTAGVSDNSEFLIPKGIQRFHFSGGARFVHGGTMLQE |
| | VCVPVLQIKALQKTAAEKQPQRRPVDIVAYHPMIKLVNNIDKVSLLQTHPVGELYEPRILNIYIVDNANNVVSGKERISFDS |
| | DNNTMEKRVREVTLKLIGANFNRRNEYWLILEDAQTETGYQKYPVIIDLAFQDDFF* (SEQ ID NO: 306) |
| 6 | MQTHHDLPVSGVSAGEIASEGYDLDALLNQHFAGRVVRKDLTKQLKEGANVPVYVLEYLLGMYCASDDDDVVEQGLQN |
| | VKRILADNYVRPDEAEKVKSLIRERGSYKIIDKVSVKLNQKKDVYEAQLSNLGIKDALVPSQMVKDNEKLLTGGIWCMIT |
| | VNYFFEEGQKTSPFSLMTLKPIQMPNMDMEEVFDARKHFNRDQWIDVLLRSVGMEPANIEQRTKWHLITRMIPFVENNYN |
| | VCELGPRGTGKSHVYKECSPNSLLVSGGQTTVANLFYNMASRQIGLVGMWDVVAFDEVAGITFKDKDGVQIMKDYMAS |
| | GSFSRGRDSIEGKASMVFVGNINQSVETLVKTSHLLAPFPTAMIDTAFFDRFHAYIPGWEIPKMRPEFFTNRYGLITDYLAEY |
| | MREMRKRSFSDAIDKFFKLGNNLNQRDVIAVRRTVSGLLKLMHPDGAYSKEDVRVCLTYAMEVRRRVKEQLKKLGGLEF |
| | FDVNFSYIDNETLEEFFVSVPEQGGSELIPAGMPKPGVVHLVTQAESGMTGLYRFETQMTAGNGKHSVSGLGSNTSAKEAI |
| | RVGFDYFKGNLNRVSAAAKFSDHEYHLHVVELHNTGPSTATSLAALIALCSILLAKPVQEQMVVLGSMTLGGVINPVQDL |
| | AASLQLAFDSGAKRVLLPMSSAMDIPTVPAELFTKFQVSFYSDPVDAVYKALGVN* (SEQ ID NO: 307) |
| 7 | MHKYPSIIVNINLREAKLKKKVREHLQSLGFTRSDSGALQAPGNTKDVIRALHSSQRAERIFANQKFITLRAAKLIKFFASGN |
| | EVIPDKISPVLERVKSGTWQGDLFRLAALTWSVPVSSGFGRRLRYLVWDESNGKLIGLIAIGDPVFNLAVRDNLIGWDTHA |
| | RSSRLVNLMDAYVLGALPPYNALLGGKLIACLLRSRDLYDDFAKVYGDTVGVISQKKKQARLLAITTTSSMGRSSVYNRL |
| | KLDGIQYLKSIGYTGGWGHFHIPDSLFIELRDYLRDMDHAYADHYMFGNGPNWRLRTTKAALNALGFRDNLMKHGIQRE |
| | VFISQLAENATSILQTGKGEPDLTSLLSAKEIAECAMARWMVPRSIRNPEYRLWKARDLFDFISNDSLNFPPFDEIAKTVV* |
| | (SEQ ID NO: 308) |
| 8 | MNYAIDKFTGTLILAARATKYAQYVCPVCKKGVNLRKGKVIPPYFAHLPGHGTSDCENFVPGNSIIVETIKTISKRYMDLRL |
| | LIPVGSNSREWSLELVLPTCNLCRAKITLDVGGRSQTLDMRSMVKSRQIGAELSVKSYRIVSYSGEPDPKFVTEVERECPGL |
| | PSEGAAVFTALGRGASKGFPRAQELRCTETFAFLWRHPVAPDFPDELEIKSLASKQGWNLALVTIPEVPSVESISWLKSFTY |
| | LPVVPARTSITAIWPFLNQKTSINHVECVYSDTILLSTNMAPTSSENVGPTMYAQGSSLLLSAVGVETSPAFFILNPGENDFV |
| | GVSGSIEQDVNLFFSFYKKNVSVPRKYPSIDLVFTKRNKEKTIVSLHQRRCIEVMMEARMFGHKLEYMSMPSGVEGVARIQ |
| | RQTESNVIKLVSNDDIAAHDKSMRLLSPVALSQLSDCLANLTCHVEIDFLGLGKIFLPGSSMLSLDDGKFIELSPNLRSRILSF |
| | ILQMGHTLHGFSLNNDFLLVEKLVDLQPEPHLLPHYRALVKEVKTNGFECNRFR* (SEQ ID NO: 309) |
| 9 | MSYQYSQEAKERISKLGQSEIVNFINEISPTLRRKAFGCLPKVPGFRAGHPTEIKEKQKRLIGYMFQSHPSSEERKAWKSFSL |
| | FWQFWAEEKIDKSFSMIDNLGLKENSGSIFIRELAKNFPKVARENIERLFIFSGFADDPDVINAFNLFPPAVVLARDIVIDTLPI |
| | RLDELEARISLIADNVEKKNNHIKELELKIDAFSEQFDNYFNNEKSSLKIINELQSLINSETKQSDIANKAIDELYHFNEKNKQ |
| | LILSLQEKLDFNALAMNDISEHEKLIKSMANDISEFKNALTILCDNKIKNNELDYVNELKKLTERIDTLEINTSQASEVSVTN |
| | RFTKFHEIAHYENYEYLSSSEDISNRISLNLQAVGLTKNSAEKLARLTLATFVSGQIIQFSGSLADIIADAIAIAIGAPRYHIWR |
| | VPVGIISDMDAFDFIETIAESSRCLLLKGANLSAFEIYGAAIRDIVVQRQIHPTNYDHLALIATWKQGPATFPDGGMLAELGP |
| | VIDTDTLKMRGLSATLPQLKPGCLAKDKWTNIDGLHLDSVDDYVDELRALLDEAGFDGGTLWKRMIHIFYTSLIRIPNGNY |
| | IYDLYSVLSFYTLTWAKIKGGPVQKIEDIANRELKNYSAKISS* (SEQ ID NO: 310) |
| 10 | MEWRAVSRDKALDMLSTALNCRFDDEGLRISAVSECLRSVLYQYSISETEEARQTVTSLRLTSAVRRKLVPLWPDIADIDN |
| | AIHPGIMSILNSLAELGDMIKLEGGNWLTAPPHAVRIDNKMAVFFGGEPSCTFSTGVVAKSAGRVRLVEEKVCTGSVEIWD |
| | ANEWIGAPAEGNEEWSSRLLSGTISGFIDAPGNMSETTAYVRGKWLHLSELSFNKKQIYLCRMSVDNHFSYYLGEIEAGRL |
| | CRMNSLESSDDVRRLRFFLDTKDNCPLKVRIKISNGLARLRLTRRLPRRETKVLLLGWRESGFENEHSGITHHVFPEEILPIV |
| | RSAFEGLGIIWINEFTRRNEI* (SEQ ID NO: 311) |
| 11 | MINKNKVTERSGIHDTVKSLSENLRKYIEAQYHIRDEGLIAERRALLQQNETIAQAPYIEATPIYEPGAPYSELPIPEAASNVL |
| | TQLSELGIGLYQRPYKHQSQALESFLGENASDLVIATGTGSGKTESFLMPIIGKLAIESSERPKSASLPGCRAILLYPMNALVN |
| | DQLARIRRLFGDSEASKILRSGRCAPVRFGAYTGRTPYPGRRSSRRDELFIKPLFDEFYNKLANNAPVRAELNRIGRWPSKD |
| | LDAFYGQSASQAKTYVSGKKTGKQFVLNNWGERLITQPEDRELMTRHEIQNRCPELLITNYSMLEYMLMRPIERNIFEQTK |
| | EWLKADEMNELILVLDEAHMYRGAGGAEVALLIRRLCARLDIPRERMRCILTSASLGSIEDGERFAQDLTGLSPTSSRKFRII |
| | EGTRESRPESQIVTSKEANALAEFDLNSFQCVAEDLESAYAAIESLAERMGWQKPMIKDHSTLRNWLFDNLTGFGPIETLIEI |
| | VSGKAVKLNILSENLFPDSPQQIAERATDALLALGCYAQRASDGRVLIPTRMHLFYRGLPGLYACIDPDCNQRLGNHSGPTI |
| | LGRLYTKPLDQCKCASKGRVYELFTHRDCGAAFIRGYVSSEMDFVWHQPNGPLSEDEDIDLVPIDILVEETPHVHSDYQDR |
| | WLHIATGRLSKQCQDEDSGYRKVFIPDRVKSGSEITFDECPVCMRKTRSAQNEPSKIMDHVTKGEAPFTTLVRTQISHQPAS |
| | RPIDGKHPNGGKKVLIFSDGRQKAARLARDIPRDIELDLFRQSIALACSKLKDINREPKPTSVLYLAFLSVLSEHDLLIFDGED |
| | SRKVVMARDEFYRDYNSDLAQAFDDSFSPQESPSRYKIALLKLLCSNYYSLSGTTVGFVEPSQLKSKKMWEDVQSKKLNIE |
| | SKDVHALAVAWIDTLLTEFAFDESIDSTLRIKAAGFYKPTWGSQGRFGKALRKTLIQYPAMGELYVEVLEEIFRTHLTLGK |
| | DGVYFLAPNALRLKIDLLHVWKQCNDCTALMPFALEHSTCLACGSNSVKTVEPSESSYINARKGFWRSPVEEVLVSNSRLL |
| | NLSVEEHTAQLSHRDRASVHATTELYELRFQDVLINDNDKPIDVLSCTTTMEVGVDIGSLVAVALRNVPPQRENYQQRAG |
| | RAGRRGASVSTVVTYSQNGPHDSYYFLNPERIVAGSPRTPEVKVNNPKIARRHVHSFLVQTFFHELMEQGIYNPAEKTAILE |
| | KALGTTRDFFHGAKDTGLNLDSFNNWVKNRILSTNGDLRTSVAAWLPPVLETGGLSASDWFAKVAEEFLNTLHGLAEIVP |
| | QTAVLVDEENEDDEQTSGGMKFAQEELLEFLFYHGLLPSYAFPTSLCSFLVEKIVKNIRGSFEVRTVQQPQQSISQALSEYA |
| | PGRLIVIDRKTYRSGGVFSNALKGELNRARKLFNNPKKFIHCDKCSFVRDPHNNQNSENTCPICGGILKVEIMIQPEVFGPEN |
| | AKELNEDDREQEITYVTAAQYPQPVDPEDFKFNNGGAHIVFTHAIDQKLVTVNRGKNEGESSGFSVCCECGAASVYDSYSP |
| | AKGAHERPYKYIATKETPRLCSGEYKRVFLGHDFRTDLLLLRITVGSPLVTDTSNAIVLRMYEDALYTIAEALRLAASRHK |
| | QLDLDPAEFGSGFRILPTIEEDTQALDLFLYDTLSGGAGYAEVAAANLDDILTATLALLESCECDTSCTDCLNHFHNQHIQS |
| | RLDRKLGASLLRYALYGMVPRCASPDIQVEKLSQLRASLELDGFQCIIKGTQEAPMIVSLNDRSIAVGSYPGLIDRPDFQHD |
| | VYKSKHTNAHIAFNEYLLRSNLPQSHQNIRKMLR* (SEQ ID NO: 312) |
| 12 | MKKVYELTSEEALSYFLRHDSYTTLELPAYINFTTLLNDINSSIHNKKIKIEPTAKELMGKDINYEVLVSKDGLYSWRRITLI |
| | NPLYYVYFCRKITAPATWEIITEKFKSFESNDLFTCSSIPVRKDNSSNIAASVMNWWEDFEQKSLALALEYEFMFSTDISNFY |
| | PSIYTHSFEWVFISKEEAKKKKSKNNPGGLIDSHIQMMMNNQTNGIPLGSTLMDTFAELILGQIDIELRKKTNELKIINYKVV |
| | RYRDDYRIFSNSKDDLDIISKCLVNVLGDFGLDLNSKKTELYEDIILHSLKQAKKDYIKEKRHKSLQKMLYSIYLFSLKHPNS |
| | KTTVRYLNDFLRNLFKRKTIKDNGQQVDAMLGIISSIMAKNPTTYPVGTAIFSKLLSFLYGDDTQKKLTKLEQLHKKLDKQ |
| | PNTEMLDIWFQRTQAKINLEWNKSYKSALCVRINDELTKEKTFSVNNLWNIDWIQGKETSPNKAKILSLLRKTKIVDTDKF |
| | DKMDDNITPEEVNLFFKEHSN* (SEQ ID NO: 313) |
| 13 | MSLHDKLLMHNFALANKKSPDFISELPQIEPKPYSNGHKIKWINHTLTSTEVTPPDNLIKICILIESGEIAITSVSDIANLLGYP |
| | AGQLLYILYRKKDNYRTFEIEKKNGKKRVINAPCGGLSILQTRLKPVLEYFYRPKKSAHGFIKGKSIITNAGMHIKKNFVVNI |
| | DLENYFESISFARVYGIFKSKPFNFAHPAATVLAQLCTHNGKLPQGACTSPILANIASASLDKQLTQFAGRKKISYSRYADDI |
| | TFSFNQRNIDIIKKNDDGSYSLSETIDNIISKNGFKINYDKFRVQTRNTRQSVTGLVVNDKVNINRRYIRITRSMIHRWTDDK |
| | LKYALLFATEKGYQAKDNNHAIQIFRNHIYGRLSFIKMVRGKDYPGYLKLMSYMSHNDPLKTQEGLRAMKETENFDVFIC |
| | HASEDKKDIAIPIYDELTKLKISAFIDHVEIKWGDSLIDKINAALVKSKYVIAILSANSVNKEWPQKELRAVLASEISSGDVKL |
| | LTLLKKEDEEVVNLSLPLLSDKFYMVYDNNPEVVANNIKSLLQR* (SEQ ID NO: 314) |
| 14 | MTKTSKLDALRAATSREDLAKILDVKLVFLTNVLYRIGSDNQYTQFTIPKKGKGVRTISAPTDRLKDIQRRICDLLSDCRDEI |
| | FAIRKISNNYSFGFERGKSIILNAYKHRGKQIILNIDLKDFFESFNFGRVRGYFLSNQDFLLNPVVATTLAKAACYNGTLPQG |
| | SPCSPIISNLICNIMDMRLAKLAKKYGCTYSRYADDITISTNKNTFPLEMATVQPEGVVLGKVLVKEIENSGFEINDSKTRLT |
| | YKTSRQEVTGLTVNRIVNIDRCYYKKTRALAHALYRTGEYKVPDENGVLVSGGLDKLEGMFGFIDQVDKFNNIKKKLNK |
| | QPDRYVLTNATLHGFKLKLNAREKAYSKFIYYKFFHGNTCPTIITEGKTDRIYLKAALHSLETSYPELFREKTDSKKKEINLN |
| | IFKSNEKTKYFLDLSGGTADLKKFVERYKNNYASYYGSVPKQPVIMVLDNDTGPSDLLNFLRNKVKSCPDDVTEMRKMK |
| | YIHVFYNLYIVLTPLSPSGEQTSMEDLFPKDILDIKIDGKKFNKNNDGDSKTEYGKHIFSMRVVRDKKRKIDFKAFCCIFDAI |
| | KDIKEHYKLMLNS* (SEQ ID NO: 315) |
| 15 | MNKKFTDEQQQQLIGHLTKKGFYRGANIKITIFLCGGDVANHQSWRHQLSQFLAKFSDVDIFYPEDLFDDLLAGQGQHSLL |
| | SLENILAEAVDVIILFPESPGSFTELGAFSNNENLRRKLICIQDAKFKSKRSFINYGPVRLLRKFNSKSVLRCSSNELKEMCDS |
| | SIDVARKLRLYKKLMASIKKVRKENKVSKDIGNILYAERFLLPCIYLLDSVNYRTLCELAFKAIKQDDVLSKIIVRSVVSRLI |
| | NERKILQMTDGYQVTALGASYVRSVFDRKTLDRLRLEIMNFENRRKSTFNYDKIPYAHP* (SEQ ID NO: 316) |
| 16 | MKSAEYLNTFRLRNLGLPVMNNLHDMSKATRISVETLRLLIYTADFRYRIYTVEKKGPEKRMRTIYQPSRELKALQGWVL |
| | RNILDKLSSSPFSIGFEKHQSILNNATPHIGANFILNIDLEDFFPSLTANKVFGVFHSLGYNRLISSVLTKICCYKNLLPQGAPSS |
| | PKLANLICSKLDYRIQGYAGSRGLIYTRYADDLTLSAQSMKKVVKARDFLFSIIPSEGLVINSKKTCISGPRSQRKVTGLVIS |
| | QEKVGIGREKYKEIRAKIHHIFCGKSSEIEHVRGWLSFILSVDSKSHRRLITYISKLEKKYGKNPLNKAKT* (SEQ ID NO: |
| | 317) |
| 17 | MSVIRGLAAVLRQSDSDISAFLVTAPRKYKVYKIPKRTTGFRVIAQPAKGLKDIQRAFVQLYSLPVHDASMAYMKGKGIRD |
| | NAAAHAGNQYLLKADLEDFFNSITPAIFWRCIEMSSAQTPQFEPQDKLFIEKILFWQPIKRRKTKLILSVGAPSSPVISNFCMY |
| | EFDNRIHAACKKVEITYTRYADDLTFSSNIPDVLKAVPSTLEVLLKDLFGSALRLNHSKTVFSSKAHNRHVTGITINNEETLS |
| | LGRDRKRFIKHLINQYKYGLLDNEDKAYLIGLLAFASHIEPSFITRMNEKYSLELMERLRGQR* (SEQ ID NO: 318) |
| 18 | MTKQYERKAKGGNLLSAFELYQRNSDKAPGLGEMLVGEWFEMCRDYIQDGHVDESGIFRPDNAFYLRRLTLKDFRRFSL |
| | LEIKLEEDLTVIIGNNGKGKTSILYAIAKTLSWFVANILKEGGSGQRLSEMTDIKNDAEDRYSDVSSTFFFGKGLKSVPIRLSR |
| | SALGTAERRDSEVKPAKDLADIWRVINEVNTINLPTFALYNVERSQPFNRNIKDNTGRREERFDAYSQTLGGAGRFDHFVE |
| | WYIYLHKRTVSDISSSIKELEQQVNDLQRTVDGGMVSVKSLLEQMKFKLSEAIERNDAAVSSRVLTESVQKSIVEKAICSVV |
| | PSISNIWVEMITGSDLVKVTNDGHDVTIDQLSDGQRVFLSLVADLARRMVMLNPLLENPLEGRGIVLIDEIELHLHPKWQQ |
| | EVILNLRSAFPNIQFIITTHSPIVLSTIEKRCIREFEPNDDGDQSFLDSPDMQTKGSENAQILEQVMNVHSTPPGIAESHWLGNF |
| | ELLLLDNSGELDNHSQVLYDQIKAHFGIDSIELKKADSLIRINKMKNKLNKIRAEKGK* (SEQ ID NO: 319) |
| 19 | MRELARLERPEILDQYIAGQNDWMEIDQSAVWPKLTEMQGGFCAYCECRLNRCHIEHFRPRGKFPALTFIWNNLFGSCGD |
| | SRKSGGWSRCGIYKDNGAGAYNADDLIKPDEENPDDYLLFLTTGEVVPAIGLTGRALKKAQETIRVFNLNGDIKLFGSRRT |
| | AVQAIMPNVEYLYTLLEEFDEDDWNEMLRDELEKIESDEYKTALKHAWTFNQEFA* (SEQ ID NO: 320) |
| 20 | MKLLDKKYYNLEPKYEYLKDSFILGLAWKKTDSFVRTHNWYADILELDKCAFDISDEVTNWSNEISKNALSKSDIELIPAP |
| | KGASWFINQGKWTTNKDNRKIRPLANISIRDQSFATAVTMCLADAIETRQKDCSLSNLGYAEHVKNKVVSYGNRLVCDW |
| | DNERARFRWGGSEYYRKFSSDYRSFLQRPIYIGRETVNKVSGIDDVYIISLDLKNFFGSIKINLLLEKIKKISADHYAAKFIND |
| | NEFWTLANRILSWDWPEESLSLLESLDIKEKNVGLPQGLASAGALANAYLIEFDESLISKLRTKIEDSQIILHDYCRYVDDIR |
| | LVISGEALESNKIKESIHALVQGILDETLAQNPSDNEPYLKINDSKTYILELSDIDNGSGLTNRINEIQHEVGASSIPERNGLDN |
| | NIPALQQLLLTEQDNFSEDVDSLFPGFKNDKSIKVESVRRFSAHRLEKSLAKKSKLISPEERKQFDNETSLIAKKLLKAWLK |
| | DPSIMVIFRKAIAINPNLDAYSTILEIIFSRIQRNRDKRDKYIMLYLLSDIFRSVIDVYRNLESEYVDDYQKLMGEVTLFAQKIL |
| | SCKSFIPNYAYQQALFYLAVINKPFIASNKASFDLARLQCVLIKQHLEPLNSSDGYLFEVSAQISKDYRANAAFLLSHTNSNK |
| | VVDLIIEKFAFRGGEFWNAIWKEIVRMQDKDRINEFRWAISKYESKPNSSEHYLSSVISFKENPFRYEHALLKLGVALVELF |
| | DDTEKNVWQPDGKQYSPHEIKVKLEGNSTSWGELWRPNFSISCSIDKKGEPGKDPRYISPEWLANYPQTQNDEQKIYWVC |
| | SVLRSAALGNVDYTQRNDLKLDKAKYDGIHSQFYKRRMGMLHTPESIVGSYGTITDWFASFLQHGLQWPGFSSSYISQEDI |
| | LSITNIIEFKNCLLERLGYLNKQICISSNVPTLPTVVNRPELASNHFRIVTVQQLFPKDTNFHPSDVTLANPDVRWKHREHLA |
| | EICKLTEQTLNAKLKTESREHTSTADLIVFSELAVHPEDEDIVRALAFRTKAIIFSGFVFCEQDGRIVNKARWIIPDSSESGTQ |
| | WRVRDQGKHHMTSDEVALGIQGYRPSQHIISIEGHPEGPFKLTGAICYDATDIKLAADLRDLTDMFVIAAYNKDVDTFDN |
| | MASALQWHMYQHIVITNTGEYGGSTMQAPYKEKYHKLISHAHGTGQIAISTADIDLAAFRRKLQIYKKTKTQPAGYNRKH* |
| | (SEQ ID NO: 321) |
| 21 | MDTLVKLATIISPLISAGVAIWAILVAKKTISESKEIAKKTIADTAYQAYLQLAMENPQFSKGYSADCRQERDPMYDQYVW |
| | YVARMIFCFEKIIEVEVNLKDSSWANTLEKHLKFHSEHFKKTNVVEEALYIPPILDLIRCAAN* (SEQ ID NO: 322) |
| 22 | MNNDDYPWFRKRGYLHFDEPVSLKKAVKYVSSPEKIIKHSFLPFLSFEVKSFKIKKDKSTKQLSKTEKLRPIAYSSHLDSHIY |
| | AFYAEYLTGHYELLIQENNLHENILAFRSLNKSNIEFAKRAFDTITEMGECSAVALDLSGFFDNLDHQILKHQWCKVIGTEA |
| | LPQDHFAIYKSITRYSKVDKNRAYEILGISKNNPKYNRRKICTPVDFRNKIRKNGLIIVNNSQKGIPQGSPISALLSNIYMLDF |
| | DIEMRDYAQERGGHYYRYCDDMLFIVPTKYNKTLAGDVAQRIKHLKVELNTKKTEIRDFIYKDSTLVANMPLQYLGFIFD |
| | GSNILLRSSSLARYSERMKRGVRLAKATMDSKNRIRENKGEALKALFKKKLYARYSHIGRRNFLTYGYRAAKIMNSKAIK |
| | RQLKPLQKRLENEILK* (SEQ ID NO: 323) |
| 23 | MLNQSFSVSNLIKLLKKTDPKRYKIGRNSAEYKKYIADKVNGSIETYSFGSISNSRINNKNVYIFKDFMDVLVARKINDNIKR |
| | VYSVKQNNRHDIIKKVNTVLSEPVNYYIYRLDIKSFYESIDKNIVFQRINNNPIISHNTKKFINGLFKHNAFSANNGLPRGMG |
| | LSATLSEIFMEEFDAELARLPEVFYASRYVDDIIVFSFYKIPDYKNYFSRILPNGLHLNERKCSEYTIEDTSTKHSEIEFLGYSFI |
| | IHHGLKNQRRHVVIRISEEKIKKIKRRIALAVKDYSNNSDAELLKKRIKYLTGNILVNSNSNKTDALYSGIYYNYQHLTDKT |
| | QLKELDIFKNRMLFSSKGEVGRKILAAGHNLLTAPKKYSFLAGFEKRLLSSFKREDIIKINKVW* (SEQ ID NO: 324) |
| 24 | MKIKISKSDYKRVLLTDILPYEVPILFSNEGFYKLISENKVLPGTFSEGLKLDSYTIPYSYKIKKGLASSRSLGIIHPSTQLRICD |
| | FYDKYEHLMVHMCTKSPFSLRYPSKIGSYYYEKDFLKSRINLKDGLVQFHNHGFDSQETSSSSHFSYKKYPFIYKFYESYEF |
| | HRLERKFRKLLKLDIAKCFSHIYTHSVSWAVKSKEFSKVNRTYNSFEGCLDKLFQDANYGETNGIIIGPEFSRIFAEIILQRVD |
| | LNVESHLNLEPGIVKDKSYAIRRYVDDYFIFADDDETFKLIEFVLANELEKYKLYLNESKKEFIERPFVTGATMAKNDIAEII |
| | EDLYGSLIHTEKLDELTAMVNLNPDVKIQPENMNDLFPLKGVWNKKLHADKFIKRIKIAVRKNNTTFDLVSSYLLSAIKSK |
| | FFKVIRLLRMFDLSGKEDITYKFFSIFNEVIFFIYAMDFRVRQTYIISQVILEINSFANKQASDISEVIKKNTFDELLMCMKSMG |
| | NIHERPVELSNLLICMKGLGEQYKLNPDEFKDLLGISENECFYDLEYFSICSMLHYIGDDVLYLKMKEDIVLAIQSLISGRND |
| | IKKDTETFMLFLDMMTCPYLTVKHKRHYRTYVEANTGQKRFTNAVIDSEIDSLKNNVIFFNWSGDADLEHVLYKKELRTA |
| | YE* (SEQ ID NO: 325) |
| 25 | MVIFDEKRHLYEALLRHNYFPNQKGSISEIPPCFSSRTFTPEIAELISSDTSGRRSLQGYDCVEYYATRYNNFPRTLSIIHPKAY |
| | SKLAKHIHDNWEEIRFIKENENSMIKPDMHADGRIIIMNYEDAETKTIRELNDGFGRRFKVNADISGCFTNIYSHSIPWAVIG |
| | VNNAKIALNTKVKNQDKHWSDKLDYFQRQAKRNETHGVPIGPATSSIVCEIILSAVDKRLRDDGFLFRRYIDDYTCYCKTH |
| | DDAKEFLHLLGMELSKYKLSLNLHKTKITNLPGTLNDNWVSLLNVNSPTKKRFTDQDLNKLSSSEVINFLDYAVQLNTQV |
| | GGGSILKYAISLVINNLDEYTITQVYDYLLNLSWHYPMLIPYLGVLIEHVYLDDGDEYKNKFNEILSMCAENKCSDGMAWT |
| | LYFCIKNNIDIDDDVIEKIICFGDCLSLCLLDSSDIYEEKINNFVSDIIKLDYEYDIDRYWLLFYQRFFKDKAPSPYNDKCFDIM |
| | KGYGVDFMPDENYKTKAESYCHVVNNPFLEDGDEIVSFNDYMAIA* (SEQ ID NO: 326) |
| 26 | MTSTIDFYESDFSATLYPLKTNQILLKHHSQEMSEYIYQKVINPAYPTDSFLSQQKVFSTKPKGHLRRTVKLDPVAEYFIYD |
| | VIYRNRKIFRPEVSESRKSFGYIFRNGSRIPIHVSYNEYKQSLKKYSELYSHSIHFDIASYFNSLYHHDIIHWFSSKEGVSPADV |
| | EALGQFFREINSGRSIDFMPQGIYPAKMIGNEFLKFVDLHGRLKSAQIVRFMDDFTIFDNDIETLNNDFIRIQQLLGQVSLNIN |
| | PSKTTFDNVMGDVNETLTQIKSSLKEIITEYEHIPTASGVEVVETNIEIIKHLDDEQVNKLIDLLKDEKIEESDADLILGFLRTH |
| | NDSLLSQMPMLLGRFPNLIKHIYTICSGITDKSGLVKILLSYLNTNNNFLEYQLFWIGAIVEDYLLGVGEYGSVLHKLYELSG |
| | DFKIARAKVLEIPEQGFGFKEIRNEYLRTGQSDWLSWSSAIGTRNLKSAERNYILDYFSKGSPINYLVASCVKKL* (SEQ ID |
| | NO: 327) |
| 27 | MTSEIVLNLDFPEYKDDFCTDSIDEQDNELWQQQANKKLLSFLEVMGEEARRYKENNSRSTHPHYKTLSSYHHAIFISGAR |
| | GAGKTVFMRNARFSWQKHYNKDLKRPKLYFIDVIDPTLLNIDDRFSEVIIASIYATVEKRMKQPDIAQNIKDNFINSLKTLS |
| | GALGKSKDYDEYRGIDRIQKYRSGIHLEKYFHQFLISSVELLDCDALVLPIDDVDMKIDNAFGVLDDIRCLLSCPLVLPLVS |
| | GDNDLYRFIAKSKFEELLNRKANSNYAKEGSEIAERLSEAYITKVFPSHVKIPLQPIDELLPYLYIHSNEDENKQHTSYSEFIK |
| | LVQQKFYFLCNGQERSTNWPQPRSAREVTQLIRSLPPSTLSKEDDSGTDLWQRFAVWAEERRDGLALTNVESYLFIKNAK |
| | AVEDLNLSNLIAFNPLLQKGKYPWAEKDFYKQQSQRRKELNAPETNSGILNTVFSEQRKDFILRSMPALELIMEPMYVTKT |
| | VAEKNDNSALIAIYTHSDYYSQQQNRRCHIFFGRAFEIMFWSVLAKTENLPQEFYEKDKFKSLFGNIFKKVPFYSIFSMNPT |
| | KVVDEENDDGSEPDFSQKLDDSINELVEDIYIWATSNKLRAFKNKNLIPLMTCVFNKVFSQINVLRKNVQDRVKFRDEHLS |
| | DLAKRFEYMFINAIFTFIREGVVVNTNVATGAAPARVRNLSEFNRYDKTLSRNMSGILSVKEDNGLTIVKESEGDIADLLFEI |
| | WHSPLFKLTTRTCYPIGKINSQNTAQENLSSDFNSFFENGINFELIKQYYWQTSNHDNIRTADVREWATSRLNEAIILFSWM |
| | KESKSIKAKIDGQSYEGRLFRGLQQALEGYEEV* (SEQ ID NO: 328) |
| 28 | MFNQDPYWLIPTLCLASDRIFYAQLRDHLGQKSSGERKKEKNGYILVQAAQDYQFYFGGRIRKEDVQNNALMWQIETGN |
| | ENCLSMLDSLSAYFLTWRGNCFEVRRERLEPWLMICSVIDPAWIIAYAYQQLIKQNVVCDSELISLLTEHQCPFAFPKGRGD |
| | ISFADNHVHLNGHGYSSISMLNFIDGNYKVKKGIKWPYRQEYTLFESGLLDKNDLPRWLSAYSSCLLKNVYNSFQQGKRS |
| | EVDFTCLKDAVETVLADEDKYYFLEVASLYDVVTLQQRVLYEAAQQKYHSHQRWLLYTCGIMLGTESEDYANALANLIR |
| | ISNILRNYMVVSAVGLGQFIDFFGFNYRRITKPADTNNRVHYDSSAGISREYRVSPDFVLGSGVMPDIYARQLFDFYCTQAR |
| | KGVPEQGHIVVHFTRSFPDKKSTYDKLLTECRERLRSQCDYFGRFLTSLTLQSIEYKNLSTDEDRSIDIRKLVRGYDVAGNE |
| | NELQIEVFAPVLRVLRAAKFKGEGVNFKRLQRPFITVHAGEDYCHILSGLRAMDEAVEFCMLGEGDRIGHGLALGVDIKL |
| | WANRQKRAYLTVGQHLDNLVWAYHQAVLLSQHIVEHIPVMHELRDKIHYWSHQLYSETYTPDLLFKAWLLRRNWPDYK |
| | SIISDPANINEWVPDQHILVSTDETTAKARKIWERYLNSGLAENDVFNRIISVNCAPDTAQNFSMTFNENEDILSKGELLLYE |
| | AIQDFLIEKYSRLGLVIEACPTSNIYIGRLEKYHEHPLFRWNPPDSQWIKPGGKFNRFGLRTGPLSVCINTDDSALMPTTIENE |
| | HRLMRDCAIHFYGIGTWMADLWINSIRIKGIEIFKGNHLSQDLDNLI* (SEQ ID NO: 329) |
| 29 | MNTIYIPLDSGESAVLKDPDTLLPRNIYEQLTRFIEKAVNEVPKPHEALNETRSHKAISIDGARGTGKTSVLVNLNDYLQSN |
| | AQQLAGKIHILDPIDPTLLEDGESLFLHIIVAAVLHDKEIKTAQSRDLDKSRVFTQKLENLAHGLESVDLQQNQRGMDKIRS |
| | LYGSKHLANCVEEFLKSALELIGKKLLILPIDDVDTSLNRAFENLEILRRYLTSPYVLPVVSGDRRLYDEVCWRDFHGRLNK |
| | DSAYNRKNTYDIARDLAIEYQRKILPLPRRLSMPDVSDYWQQDGIEVTLDKNGIPLRNFMAWLKIFITGPVNGLEGSDLPLP |
| | IPSIRALTQFINHCRDLIRELPEPFRKKVSTLALRRMWQMPDVPLDVLESFAEKHRELSKEAKREYGEAYKLFYDGLKNFTA |
| | WDSKAYLEDDKQSAWLDRLCEYFRFEPKAGAVFLTLQAKQFWVSWAQGDNRNQSILATPLFQPLLHNFREYDVFERYDD |
| | LSDWESQLRTRLPESWLTAIKGQKTLLPYPVAEAGINTSLKWRYWEELENYGFDPALESKANFLLSTLMQRNFYTNSKQS |
| | VVINIGRVFEIIIASLVSDLELADLQRIRQRSPFYSASALAPTKTLDLEEDFTKKNTRFMNNRSETDRDISDDILVDVPDKNED |
| | AWKKICDEINHWRKTHNVASTNLSPWLVYKVFNKTYSQVANNVFVPSGMQNVDAALNVFGRVFYAVWSAFGSFEKGEL |
| | FGLSDVVATTNIISAKNFYNHDNFRVNVGPFTPEQNQNSDSDREAYQHRKMYGEKTRAVSYVLATHPLKKWIDEVLRTEF |
| | KQKQNAQIQTERKMPIQAEKIIDISPAREFITRKLSLNSHSRLVKTRIIKQLKMLYPNYDKAKDFIDEVTNHFPQNDPAINTLQ |
| | KAFAELYPDGDK* (SEQ ID NO: 330) |
| 30 | MLTRSLSEHAAGCFFTDERLSQRFLDILLSPPKDFETWSSLQEESFKLLVKSIDSRYPRTYRLTDVRQLVGNICDNGLLTSPT |
| | LPWLDVIADQLLLRNGDLLYYRENKVQDYVRIAAELDPALLVGWRLGDWLLQSPPPRLTDITRVVMAQNPFFAPPANAG |
| | KPFAEGHVHLGGVTAGDTILDGYLFEEIELPKSKDMLLWAHKEHDELTPLINRAKSLLTVLLSAPPQTVSEQTQNGFDQRK |
| | TVSEKYKALQNPMDSIHRLPDWLLLAKKNRGTESVSPGWFLNQLAHASEKKHPSRWLWLQLYLCHSYQLKDTHPLERTA |
| | ILCFWLTVNALRRHIIMDGQGLACFTERYFNGALRAGKKADSSNMRYLFAGKDDVAEVKASPKAFDHEMVTGFSSTLLKT |
| | LGIPAVFPPYIFGEHEIKPDERVLRYIGALERWQFCGHFSRSKTASRGKRAKADLQANWTEAERLLQKLYSHNGWNHPVFL |
| | GGKRNPHFHFQPSNWFRGLDVAGDENVLKIAGFAPMLRWLRSGLYPVPEGLRASMSFHFSIHAGEDYAHPASGLRHIDET |
| | VRFCEMREGDRLGHALALGIEPALWAKRHGEMILPLDEHLDNLVWQWHYATLLSASLPLAQAVLPLLERRIARFIARCEW |
| | CKKRPPQIDNSVVGKQACSDDKPLENITPDTLYRAWLLRRNCSYRLQQLHGGSPLTSQEKCALPDWATLSDKGNVAAQLY |
| | QQRHSSLLDDMPPQLVVVRVADEWGTQELIGLGNPGKLRQQALDGKDILQDIDTPVELQFMHALQDYLLDHYDRKGLIIE |
| | TNPTSNVYIARFKKHVEHPIFRWNPPDEELLKPGAEFNRYGLRRGPVRVLVNTDDPGIMPTTLRTEFLLLREAAIERGVSRT |
| | MAEYWLERLRLYGLEQFQRNHLNVFEVIE* (SEQ ID NO: 331) |
| 31 | MSGTFPYLQYTDVNGLQPKLKEELKNLRRKEYLSYWPRFLIRRISLYALPFLMFFTFFFCLSLTKKVGAEEVTNILGTVSISF |
| | SSCLLLGIIISGVVLLLQWTCFNCKYSPQDTNGVVGARKLNYKLLAHVVFVIACVLLFVFIYCTNNKVFYGFIVFLGLTLLPL |
| | VIDRTLGVTRQNERHKLYIRRLERLDELNILREKMNIKFEESHFIEYMKLVDEADHGKNQDTVSDTSYFMTLIENKLKV* |
| | (SEQ ID NO; 332) |
| 32 | MKIVSNTVWDGLKLPDYRARFFIEVWKEILYVNTPSFYQSKMINTMSGAEELVEAIDDYIQDDKSKKSLLSMIEDYKGNLK |
| | KDSIAKDTFKNLHATLLKKIETVPDPISSNYILELKTIVKLVLSKESDYYHELKKQLKSSILSNADLNKKARLMDSIYQLTKS |
| | FIGYLLWKGYSPTYLYNRMEYLTRIKNYGSRDFSAQFNSCLDKLTIRIHDYTVYFLITPLSKYLIELNNILDVSFINREGIINEK |
| | NYNKISQGVESSVLAKIVVNTTDYVSAAWQANEKLDKVIDYLEIEKPEYNIRYSPVCLTEFSNGRFTHRQTINIGRLKQFITS |
| | KNYSILENIPNESKVLLRESIKLDRYDVLTRSLRYLRVAKESTSLEQKLLGVWIALECIFESTSGNIISGITNHIPTFYSTQSLEI |
| | RIRYSKDLLEARLKPISDSLLEITANQKSKFRDLSLKEYFDIVKIEKNRNKIFDELVSKGDEFAVFRLIKIFESFGTSKKINDRF |
| | NDTKKDVESQLYRIYKVRNKITHRAYYGNIRPQLVDHLYSYLLSAYSTLIYSLRYNAINKFEPQDMFNAYIISCESLIFNVEE |
| | EKKLENITMDEIILS* (SEQ ID NO: 333) |
| 33 | MVAIKMYPAKDGDAFLIICDEEKSAFLIDGGYAETFRQHILPDLRELSFNGYRLRLVMATHIDSDHIGGLVDFFLVNGHAAE |
| | PAVITVDRVWHNSLRAMTRPENNAQKVDSREITDFLRRRYHVEADKAKPHEISARQGSSLAASLLAGDYHWNEGKGYQC |
| | ICTGTSIPNLMCDNSLTILSPSKERISALCLWWRRQLASLGFSGRSSSSEAFDDAFEFFCKREASQVPLPHVINARTPLLERDY |
| | ARDTSPTNGSSIAFSLVLNKKRILMLGDAWAEEVVTSLGASGASHHFDIIKISHHGSIRNTSPNLLKIIDAPVYLISTDGKKHA |
| | RHPNLAVLKAIVDRPAAFTRTLYFNYANSASAFMKNYLSASGAQFRIIEGSTDWITL* (SEQ ID NO: 334) |
| 34 | MRYAATETEIRNATVLIECAGYTGSGTLIAADKVLTAAHCVVSDDPETPITVTFFGADEDVCVNATISEIDTSCDACLLTLS |
| | DSVDIPPITLMTQPEREGSQWKAFGYPASRNGPSHYLHGTISQILPRLFHGVDMDLSVSADCVLEEYSGVSGAAILSENKCI |
| | AMVRIRMDGGLGAVSLDKLSGLLIRNGLIPDDIASLPDSSLSGEVVLNRTEFRDNFESFVLEHKGRAVLLEGSPGSGKTTFC |
| | RHYQPRSEQLAVAGVYEFTPEDGAGTTFKILPEVFADWLHNQVSILLSGRPARREETEKINLTQKVSDLLHTFSDYWKHKG |
| | KYGVIFIDAVNEASECGDEAVSRFTALLPVTLPENVKLVFTAPSLSSAGKAFRHWLTPQDCISLTLLSHREVLQLTARELKT |
| | SAPSLSLLTRVSDIAQGHPLYLRYILGYLKANPDQVNLEIFPVFSGSIETYYERLWQGLVKDESAVNLLGILSRMRWGIDISS |
| | LIPVLTPQEQTVFVPTLDRIQHLLLNDKSSALCHQSFAAFINSKTAVINSLLHGRLADFCLTSGESYGLINRAYHLLLASHDR |
| | HPEAALVCTQEWADACIVKGAQPDILIHDIRQTLKNTLIRADAVASIRLLLLFQRMTFRHHFLFLQSAYHSGLALAALGRPD |
| | EALEQLIPSGSLVVDAVDAIVSAQTLARMGNSEHALKLLEKVKSAVDQEFERNPVNLSDFIGLSLAWVRAELMAGVVDGH |
| | GRTREVVEYLYGCGQVVRDNFEQSAHSKSAYTRAFYPLQAEMEAVNIAFNDRSVSLRTVKEKFGSLPENILDLMLSSVMR |
| | AHDIILQHQLPMPQHALQPVWYNLDRLLHTDIPYSNEIRFNSLSSLIFFNAPSALIIRMAGVFSFEVVPEITLLNEENEIAADSI |
| | DVSEQGQLWLVSAYLNETQPCPDIKHPSQGCSEWLKTLTEAIFWYSGQARRAVIDGNDEKKELLLVKVQNDILPALSYSLE |
| | ERMAWPNSWAMPEQIIPMIYEELVNMFGACWPDKISVITDFILAHTPQQCGLYSEGYRRLLNRVIQTLLNEHRFLGQSDTTF |
| | QLLETLHAFVSAFTENRQELVPELLNIIPAYISLDAPQLAQDTYTELLGVSMGPDWYKEDQFALMTTMLRVIPQHTDTNTT |
| | LSQVAGFLEHASGEMTFRRYVRQEKSQFIGELIRRGNYAHGFNYYRQQSCGSHEEMLTQLSHPAADSPHPLKGMRFPGGA |
| | LDEEHAVECIVSELRNRVDWRLRWGLLEIFSFGSIGNLAVPFAELINEFSADTEDLNEIPKRLHNILHGDVPFSEHRNFIKNFT |
| | EHLADNHKPLFAEFISLLSEDTSDNDVKPPPSGDANQKGTDTSDDVAMQPGLFGKRSAINRAEACMENARKAAARRNTVR |
| | ASELAVESLHIIQDGDWSVWRKNNHLAELTRTYILDNSADAGSVIRAYASLVEKERYAPAWVIASHLIEIAASKFSDQEAQ |
| | AINQIVLEHNRHMLGNTEADAAHFSFLNEPDTSDAGEETLYFLFWLLEHPLKFRRERALEVLKWLASDDDKILGQCVTEAL |
| | VSDIASRAEALMALTDWVSARSPQRIWDFIVKERSLFEWLEGTTALSQVHLLERVTSRAGFVLRNEIAAFERPRKLLLTSEA |
| | SGQRNIPENLPTWVQSLSQTLAVMEKQGIDIPALLTLLEKRVLQQSGLADITVAFELEKLLARGFTVNRTPSHHRWETMVR |
| | FALNQIIHEAAAQDELQNIEPLLRAWNPASEECVEPWEVCNRAKQIICAVMEGRHQQASGIEDGFFLHYLDEVEVSREGQT |
| | HLVEISAVLTTAHNGHESLRPGAESEFNATQTPDIERTLSVHLTCQRVKMQPLLFGGATPAAVSKKFMQMTGTLPSDFIRR |
| | QWRSGRSLSKNRWGEPISRGSLLLMKRTTTLPPGLGLAWYVTVDGKLMNIFSYAPRRR* (SEQ ID NO: 335) |
| 35 | MKYSSMETPKTREEFEARCFHLLNAIKLGRYHGIPGEGNKEQVPFLPNGRVDLANIDTMTRLSMNSLYDFHYNRDNYPQF |
| | DLSENDENEEATD* (SEQ ID NO: 336) |
| 36 | MEPISITVATYVATKLIDQFISQEGYGCIKKALFPQKRYVDRLYQLIEETAIEFEETYPVESGAIPFYHSEPLFEMLNEHIFFKE |
| | FPDKEILLDKFKEYPSITPPTQQQLSLFYEMLSLKINNCSKLKKLHIEETYKEKIFDINEELIQVKLILRSIDEKLTFHLSDDWL |
| | NEKNSQAIADLGGRYTPELNVKLEIAEIFDGLGRTNDFSKIFYSHIDSFLVAGKKLHSCDVISSELFEINQSLKEISDIYQEINF |
| | SKLDEIPINKFNNYVSSCQTAIGGAVSILWELREKSEQVGETKHYSDKYSSTLRMLREFDYACNELRIFINSTTVKLANNPFL |
| | LLEGKAGIGKSHLLADVIKNRIASGYPSLLILGQQLTSDESPWSQIFKRLQLKITSREFLEKLNLYGKKTGKRVLVFIDAINEG |
| | NGNKFWNDNINSFVDEIRCFEWLGLIMSVRTTYRNVTISHENVVRNNFEIHEHIGFQNVELEAVSLFYDYYNIERPSSPNLN |
| | PEFKNPLFLKLLCEGIKKNGLTKVPVGFNGISNIFNFLVEGVNKSLASPKKYAFDPSFPLVKDALNEIIKFKLEIGRNSISLKD |
| | AHSVVQSVVNDYVADKTFLSALIDEGLLTKGIVRNDDNSTEEVVYVAFERFDDHLTVNFLLNDVENIESEFKPDGRLKKYF |
| | HDECDFYIKSGIVEALSIQLPERYEKELYEFLPEFSNNLKLLEAFIDSLIWRDIKAIDFEKIRPFINEHVFKFKDSFDHFLEAVISI |
| | SGLVGHPFNANFLHDWLKDYSLANRDSFWTTELKYKYSEDSAFRHLIDWAWARTDKSFVSDESIELVATSLCWFLTSSNR |
| | ELRDCSTKALVSLLEPRIPVLRKIIDKFYGVNDPYVWERIFAVALGCTLRTDNIKELKYLAETVYQKVFCSKYVYPNILLRD |
| | YAREIIEFANHLGLELESIELSKTRPPYNSIWPDKIPSKEELESLYDKEPYRELWSSIMEDGDFSRYTIGTNYNHSDWSGCKFN |
| | ETPVDRKQVFKTFKCKLTDQQKDLYDATDPFIYDDKCEGIKFGRVVGRKAQEEIKASKKLFKNSLSYDLLSEFENEIEPYLD |
| | HNNNLLETDKHFDLRLAQQFIFNRVIELGWDPEKHGNFDQQIGTGRGRREAFQERIGKKYQWIAYYEYMARLADNFTRFE |
| | GYGDERKENPYQGPWEPYVRDIDPTILLKETGTKKISNKEMWWLNDEVFDWTCSNEDWVKSSTTITNSYAFIEVKDDNGD |
| | EWIVLESHPSWKEPKIIGNDDWGHPRKEVWYQIRSYIVKVEEFENFRCWAIAQDFMGRWMPECTDRYQLFNREYYWSEA |
| | FKSFKSDYYGGSDWTSVTDRESGAKIADVSVTSINYLWEEEFDKSKIETLNFLKPSNLIFEKMGLKSGEVEGSFNDENGTM |
| | VCFAAEAVYASKPHLLVKKEPFLTMLRDNGFEIVWTLLGEKGVIGGSLISSHHYGRQEFSGAFYYEDSQLTGSHKTSFTR* |
| | (SEQ ID NO: 337) |
| 37 | MSDSLLVRTSRDGDQFHYLWAARRALRLLEPQSTLVALTIEGASTTEMGSQPVVEDGEELIDIAEYYGSNELATATTVRYM |
| | QLKHSTMHSDTPFPPSGLQKTIEGFATRYKALIQKIPVETLRTKLEFWFVTNRPVSSSFSEAINDAANQHVTRHPHDLAKLE |
| | KFTGLQGAELSIFCQLLHIEGQQDDLWSQRNILLRESAGYLPDLDTEAPLKLKELVNRKALTESAANPSITRMDVLRALGV |
| | DETDLFPAPCRIERIENSVSRTQEATLVQRVVEAFGAPVIIHADAGVGKSIFSTHIEEHLPTGSVSILYDCFGLGQYRNASSYR |
| | HHHRTALVQMANEMASRGLCHPLIPNAGTGISQYMRAFLHRLSQSISILRASEPLAVLCIIIDAADNAQMAAEEIGETRSFIK |
| | DLIREKLPDGVCLVALCRPYRRELLDPPPEALTLSLQTFNRDETAAHLHQKFPDASESDVDEFHRLSSCNPRVQALSLSQNL |
| | PLNDTLRLLGPNPKTVEDTIGEVLEKSIARLRDTAGISERAQIDTICSALAILRPLIPLSVLSAISGVAGSAIKSFALDLGRPLIV |
| | SGETIQFFDEPAETWFQRRFRPSAADLHQFITKLRPLTKDSSYAASVLPALMLEGNQLSELIELAISSQALPETSAVERRDIEL |
| | QRLQFALKAALRTGRYQDAAKLALKAGGECAGDNRQRVLLRDNIDLAAKFVGSNGVQELVSRNAFPDTGWPGSRNAYY |
| | AAILSEYPELSGEARSRLRLTMEWLTNWSQLPDDERSRQNVTDQDRAVMLIACLNIHGAEAAARELRRWRPRKLSFDAGK |
| | IVAMQLLAHARYDELDQLAIAAGNDISLVMGIVLEARKLHRPVAEQAIRRTWRLLKSQRVSIKDRNHANNQTIAAITGMV |
| | EMALIQSVCTESESIQLLDRYLPKVPPYALTSEYSKERVAYVRAYALQANLMGSQLALSDLASTEVKKELMAEKRHGESD |
| | DLRQLKQYSGVLIPWYNLWAKVILGKTRKADLESELSDTQKESTAIKGHSYSEHSLSSNEIANVWFDILIEAGNVSKDDVE |
| | NIIKWSQHKGNRVFTPTLHRFSSVCAEISGLGELSYHFAELALSLWRDEHSDAQIKADGYIDLSRSLISLDEPEAKEYFNQAI |
| | EVTNKLGDENLSRWEAILDLAEYVAGKTQVPPEISYKLARCAELTREYVDRDKHFAWSDTVEILAELCPSSALAIISRWRD |
| | RTFGNHRSILAWTIEHLVKKNKINALDALPLITFENDWHKCDLLDSVLSSCTDDKDKIMAFEVVYHYTKFNVQNIQNLKKL |
| | DAISTSLGIEHTELKERISGLQHTETVSKKSSLSSNDNEQGHDQEWESIFKDCDLSSIDGISAAYEKFRNVPEFYSKETFIKKAI |
| | SRVKTGKECSFITAIGAIFHWGLYDFKYILESIPDEWTSRLSIKTTLAGLIKEYCQRFCMRIRKSRVYEIFPFSLASRLSGISEKE |
| | IFGITLEAIAESPEPANSDRLFSLPGLLVSKLESNEALDVLSYALDLFDEVLKDEDGDGPWNEKLSPPTHVEDSLAGYIWARL |
| | GSPEAEMRWQAAHAVLALCRMSRTCVIQGIFQHAINATTLPFCDRNLPFYTLHAQLWLMIAAARVALDDGKSLIPNIGYFY |
| | HYATTDQPHVLIRHFAARTLLALHDSDLISIPAQEENKLRNINQSTTLPVLDKVEDHRGEDSYTFGIDFGPYWLKPLGRCFG |
| | VSQKQLEPEMLRIIRDVLGFKGSRNWDEDERNKRRYYQDRDNHHSHGSYPRVDDYHFYLSYHAMFMTAGQLLATKPLV |
| | GSDYDDVEDVFQDWLRRHDISRNDHRWLADRRDIPPKERSSWLNSSSDNRDEWLASISENVFNETLCPSPGLLTLWGRWS |
| | DVCSDRKESIIVHSALVSPERSLSLLRALQTTKNVYDYKIPDAGDNLEIDHAHYQLKGWIKDIAEYCGIDEFDPWAGNVRFP |
| | IPEPASFIIDAMKLTTDKDHRVWYSPSDVEPAMISSIWGHLSGKNDEEKSHGYRLCASIHFIKSALETFNMDLILEVDVDRYS |
| | RNSRYERNNENELDNIPSSTRLFLFRHDGTIHTLYGNYRNGEKTS* (SEQ ID NO: 338) |
| 38 | MAHHIAELIYDAEHCTDDIVRTAKQAEIRDSIWSFWSNRYELPIGSRPFQELEPILRTLKGLDPENEQPRFFSPYRDLINVEKE |
| | TSEVQKWLTAAKDIDSAAKILIDYCLSLAAENAIDKSQEWVELAQKAGLNKDVDLLEIRIFQLRGTPANTDNPNNAQRRIL |
| | EKRQKRLEAFLLLGSQLNEQLKSQLEALPAIEDEPTDDDEDF* (SEQ ID NO: 339) |
| 39 | MVKPNWDNFKAKFSENPQGNFEWFCYLLFCQEFKMPAGIFRYKNQSGIETNPITKDNEHGWQSKFYDTKLSDNKADLIEM |
| | IEKSKKAYPGLSKIIFYTNQEWGQGRKSHEPEGDKNADNYLETVGNSNDPKIKIEVDQKAYESGIEIVWRVASFFESPFVIVE |
| | NEKIAKHFFSLNESIFDLLEEKRKHTENVLYEIQTNIEFKDRSIEIDRRHCIELLHENLVQKKIVIVSGEGGVGKTAVIKKIYEA |
| | EKQYTPFYVFKASEFKKDSINELFGAHGLDDFSNAHQDELRKVIVVDSAEKLLELTNIDPFKEFLTVLIKDKWQVVFTTRN |
| | NYLADLNYAFIDIYKITPGNLVIKNLERGELIELSDNNGFSLPQDVRLLELIKNPFYLSEYLRFYTGESIDYVSFKEKLWNKII |
| | VKNKPSREQCFLATAFQRASEGQFFVSPACDTGILDELVKDGIVGYEAAGYFITHDIYEEWALEKKISVDYIRKANNNEFFE |
| | KIGESLPVRRSFRNWISERLLLDDQSIKPFIAEIVCGEGISNFWKDELWVAVLLSDNSSIFFNYFKRYLLSSDQNLLKRLTFLL |
| | RLACKDVDYDLLKQLGVSNSDLLSIKYVLTKPKGTGWQSVIQFIYENLDEIGIRNINFILPVIQEWNQRNKVGETTRLSSLIA |
| | LKYYQWTIDEDVYLSGRDNEKNILHTILHGAAMIKPEMEEVLVKVLKNRWKEHGTPYFDLMTLILTDLDSYPVWASLPEY |
| | VLQLADLFWYRPLKETGERYHSMDIEDEFGLFRSHHDYYPESPYQTPIYWLLQSQFKKTIDFILDFTNKTTICFAHSHFAKN |
| | EIEEVDVFIEEGKFIKQYICNRLWCSYRGTQVSTYLLSSIHMALEKFFLENFKNADSKVLESWLLFLLRNTKSASISAVVTSIV |
| | LAFPEKTFNVAKVLFQTKDFFRFDMNRMVLDRTHKSSLISLRDGFGGTDYRNSLHEEDRIKACDDVHRNTYLENLALHYQ |
| | IFRSENVTEKDAIERQQVLWDIFDKYYNQLPDEAQETEADKTWRLCLARMDRRKMKITTKEKDEGIEISFNPEIDPKLKQYS |
| | EEAIKKNSEHMKYVTLKLWASYKREKDERYKNYGMYEDNPQIALQETKEIIKKLNEEGGEDFRLLNGNIPADVCSVLLLD |
| | YFNQLNNEEREYCKDIVLAYSKLPLKEGYNYQVQDGTTSAISALPVIYHNYPMERETIKTILLLTLFNDHSIGMAGGRYSVF |
| | PSMVIHKLWLDYFDDMQSLLFGFLILKPKYVILSRKIIHESYRQVDYDIKKININKVFLNNYKHCISNVIDNKISIDDLGSMD |
| | KVDLHILNTAFQLIPVDTVNIEHKKLVSLIVKRFSTSLLSSVREDRVDYALRQSFLERFAYFTLHAPVSDIPDYIKPFLDGFNG |
| | SEPISELFKKFILVEDRLNTYAKFWKVWDLFFDKVVTLCKDGDRYWYVDKIIKSYLFAESPWKENSNGWHTFKDSNSQFF |
| | CDVSRTMGHCPSTLYSLAKSLNNIASCYLNQGITWLSEILSVNKKLWEKKLENDTVYYLECLVRRYINNERERIRRTKQLK |
| | QEVLVILDFLVEKGSVVGYMSRENIL* (SEQ ID NO: 340) |
| 40 | MQVQHHTEPNLKNEIVALFKASQLIPFFGSGFTRDIRAKNGKVPDAIKFTELIRNIAAEKEGLTQTEIDEILRISQLKKAFGLL |
| | NMEEYIPKRKSKALLGNIFSECKLSDHEKTKIINLDWPHIFTFNIDDAIENVNRKYKILHPNRAVQREFISANKCLFKIHGDIT |
| | EFIKYEDQNLIFTWREYAHSIEENKSMLSFLSEEAKNSAFLFIGCSLDGELDLMHLSRSTPFKKSIYLKKGYLNLEEKIALSEY |
| | GIEKVITFDTYDQIYQWLNNTLQNVERKSPTRSFELDDSKLMKEEAINLFANGGPVTKIVDNKRILRNSITFSQRDVCDDAIK |
| | ALRNHDYILITGRRFSGKSVLLFQIIEAKKEYNASYYSSTDTFDPSIKNSLIKFENHIFVFDSNFFNAQSIDEILTTRVHPSNKV |
| | VLCSSFGDAELYRFKLKDKKILHTEIQIKNNLINEEGNYLNDKLSFEGLPLYKSSETLLNFAYRYYSEYKNRLSGSNLFNKQ |
| | FDEDSMFVLILIAAFNKATYGHINSHNKYFDIQNFISQNDRLFELESTNTDPSGVIICNSPSWLLRVISEYIDKNPASYKTVSD |
| | LHSLASKGFLAASRNLISFDKLNELGNGKNVHKFIRGIYKEIAHTYREDMHYWLQRAKSELISAHTIDDLVEGMSYASKVR |
| | LDSAEFKNQTYYSATLVLAQLSARALSINNDKIYALSFFESSLESIRNYNNNSRHINKMMDKNDGGFRYAIQYLKDNPLIEL |
| | LPRKDEVNELINFYESRKK* (SEQ ID NO: 341) |
| 41 | MQFITNGPDIPDELLQAHEEGRVVFFCGAGISYPAGLPGFKGLVELIYQRNGTTLSEIEREVFERGQFDGTLDLLERRLPGQR |
| | IAVRRALEKALKPKLRRRGAIDTQAALLRLARSREGALRLVTTNFDRLFHVAAKRTGQAFQAYVAPMLPIPKNSRWDGLV |
| | YLHGLLPEKADDTALNRLVVTSGDFGLAYLTERWAARFVSELFRNYVVCFVGYSINDPVLRYMMDALAADRRLGEVTPQ |
| | VWALGECEPGQEHRKAIEWEAKGVTPILYTVPAGSTDHSVLHQTLHAWADTYRDGIQGKKAIVVKHALARPQDSTRQDD |
| | FVGRMLWALSDKSGLPAKRFAELNPAPPLDWLLKAFSDERFKYSDLPRFCVSPHVEIDPKLRFSLVQRPAPYELAPQMSLV |
| | SGCVSASKWDDVMSHIARWLVRYLGDPRLIIWIAERGGQIHDRWMFLIESELDRLAALMRERKTSELDEILLHSPLAIPGPP |
| | MSTLWRLLLSGRVKSPLQNLDLYRWQNRLKNEGLTTTLRLELRGLLSPKVMLRRPFRYSEDDSSSTDEPLRIKQLVDWEL |
| | VLTADYVRSTLFDLADESWKSSLPYLLEDFQQLLRDALDLLRELGESDDRHDRSHWDLPSITPHWQNRGFRDWVSLIELLR |
| | DSWLAVRAKDSDQASRIAQNWFELPYPTFKRLALFAASQDNCIPPERWVNWLLEDGSWWLWATDTRREVFRLFVLQGR |
| | HLTGIAQERLETAILAGPPREMYEDNLEADRWHYLVAHSVWLCLAKLRGAGLVLGESAATRLTEISTAYPKWQLATNERD |
| | EFSHWMSGTGDPGFEESIDVDIAPRKWQELVQWLAKPMPERLPFYEDTWSDVCRTRFFHSLYALRKLSQDDVWPVGRWR |
| | EALQTWAEPGMILRSWRYAAPLVLDMPDAVLQEISHAVTWWMEEASKTILCHEEILLALCRRVLMIETSPESSTIRNGIETY |
| | DPVSTAINHPIGHVTQSLITLWFKQNPNDNDLLPVELKTLFTKLCNVQIELFRHGRVLLGSRLIAFFRVDRPWTEQYLLPLFA |
| | WSNPVEAKAVWEGFLWSPRLYEPLLIAFKSDFLESANHYSDLGEHRQQFAIFLTYAALGPTEGYTVEEFRTAISALPQEGLE |
| | VAAQALYQALEGAGDQREEYWKNRVQPFWQQVWPKSRNLATPRISESLTRMVIAARGEFPAALAVVQDWLQPLEHLSY |
| | DVRLLLESDICSRYPADALSLLNAVIAEQHWGPRELGQCLLQIVQAAPQLEQDVRYQRLNEYSRRRSV* (SEQ ID NO: 342) |
| 42 | MTNKNKIKPLLNNISARLWDGRAAILIGAGFSRNAKPLTSKARKFPMWNDLGDIFYESVYCKKNDNRYSNVLKLGDEVQA |
| | AFGRATLDKLIMDHVPDKEYEPSKLHVSLLSLPWIDVFTTNYDTLLERASVNVDSRKYDIVLNKNDLMNAERPRIIKLHGS |
| | FPSERPFIVTEEDYRKYPLENSPFVNTVQQSLIENTLCLIGFSGDDPNFLNWIGWIRDNLGTENSPKIYLIGLFSFNEAQRKLL |
| | EKRNISIVDLSFLGDFGKDHYLAHQRFIQFLYESKNRDNLIEWPIETNYDRIVFNDGIELKTEKIKKCILEWAQSRQSYPNWL |
| | ILPESNRSNLWQNTIDWLSVANYDVAWDGSDDLDFGYEITWRLNKALLPIFNDTSEFLFKLIEKYEINYVSGINNKIIDFDEK |
| | YSHITLSLMRFCRQENLIDKWKNLNDLLIQNLDRLTPEVKSDYYYENILFSYFNLNFDEARNKLSNWETNKLLPHHEIKRA |
| | GLLAEFGMLDEAINLLEETLSTIRRNSLLSSRNIDYSSESQEAYGIYILRMFKRSLRLDSKDDDYSSEYNSRLATLSQYRSDPE |
| | NEIKYLEIKLESLPGTFKNTNDTDFDLNKRTVTTYLGGSPTEVRSLDAFSFFLLAEELGLPFHIPGMNIFSGIVENAARHIYQY |
| | SPEWAIFSIFRTFNKDKAKSLFNRNRISSLERKKVEDLFDGYYKKYEQIITKKIEDRLNDKLEIEISTLSIIPEILSRLVTKVSFN |
| | KKKDIIHLLLKLFNSDNFHQYMETKDLLKRTISNLSDLQKISLIDIFIDFPSAPPNTQLHMGQRYNFLTPFECLLGVTITPPKEN |
| | SKKIASAKLKKDINDLKSDNLDLRKAVSQKLITLYNLEMLNKSDTTKLIKNLWSKRDNFGFPIGSGYYKFFFINNLNPDNEN |
| | IADKFISIIKTYKFPVQEGKRVSITGGLDEYCTELNGALHHISLPEKTLSEIISKIHDWYVKDRAWLEKRDDLAKEFTLRFRNI |
| | TNIITTILEHHKDKLHAESINEISSLLDKMKEDKIPVNSAVTMLCLKNKSTYLERIKDIENGLYSFNKDDVIEAINSTYVFIRN |
| | NEFPLTIIQAISDKIAWDRNPRLPDCYNLIAYIINSCEFTLPDYLIEKILRGLAYQINIDDRDFVDNNEYLNHLEKKLSATKLA |
| | ASMFRKNETLGIDQPSIIQEWKNMCNSRNEFDEIRNEWNNNI* (SEQ ID NO: 343) |
| 43 | MSIYQGGNKLNEDDFRSHVYSLCQLDNVGVLLGAGASVGCGGKTMKDVWKSFKQNYPELLGALIDKYLLVSQIDSDNNL |
| | VNVELLIDEATKFLSVAKTRRCEDEEEEFRKILSSLYKEVTKAALLTGEQFREKNQGKKDAFKYHKELISKLISNRQPGQSA |
| | PAIFTTNYDLALEWAAEDLGIQLFNGFSGLHTRQFYPQNFDLAFRNVNAKGEARFGHYHAYLYKLHGSLTWYQNDSLTV |
| | NEVSASQAYDEYINDIINKDDFYRGQHLIYPGANKYSHTIGFVYGEMFRRFGEFISKPQTALFINGFGFGDYHINRIILGALLN |
| | PSFHVVIYYPELKEAITKVSKGGGSEAEKAIVTLKNMAFNQVTVVGGGSKAYFNSFVEHLPYPVLFPRDNIVDELVEAIANL |
| | SKGEGNVPF* (SEQ ID NO: 344) |
| 44 | MSLFKLTEISAIGYVVGLEGERIRINLHEGLQGRLASHRKGVSSVTQPGDLIGFDAGNILVVARVTDMAFVEADKAHKANV |
| | GTSDLADIPLRQIIAYAIGFVKRELNGYVFISEDWRLPALGSSAVPLTSDFLNIIYSIDKEELPKAVELGVDSRTKTVKIFASV |
| | DKLLSRHLAVLGSTGYGKSNFNALLTRKVSEKYPNSRIVIFDINGEYAQAFTGIPNVKHTILGESPNVDSLEKKQQKGELYS |
| | EEYYCYKKIPYQALGFAGLIKLLRPSDKTQLPALRNALSAINRTHFKSRNIYLEKDDGETFLLYDDCRDTNQSKLAEWLDL |
| | LRRRRLKRTNVWPPFKSLATLVAEFGCVAADRSNGSKRDAFGFSNVLPLVKIIQQLAEDIRFKSIVNLNGGGELADGGTHW |
| | DKAMSDEVDYFFGKEKGQENDWNVHIVNMKNLAQDHAPMLLSALLEMFAEILFRRGQERSYPTVLLLEEAHHYLRDPYA |
| | EIDSQIKAYERLAKEGRKFKCSLIVSTQRPSELSPTVLAMCSNWFSLRLTNERDLQALRYAMESGNEQILKQISGLPRGDAV |
| | AFGSAFNLPVRISINQARPGPKSSDAVFSEEWANCTELRC* (SEQ ID NO: 345) |
| 45 | MDRSAVDTIRGYCYQVDKTIIEIFSLPQMDDSIDIECIEDVDVYNDGHLTAIQCKYYESTDYNHSVISKPIRLMLSHFKDNKE |
| | KGANYYLYGHYKSGQEKLTLPLKVDFFKSNFLTYTEKKIKHEYHIENGLTEEDLQAFLDRLVININAKSFDDQKKETIQIIK |
| | NHFQCEDYEAEHYLYSNAFRKTYDISCNKKDRRIKKSDFVESINKSKVLFNIWFYQYEGRKEYLRKLKESFIRRSVNTSPYA |
| | RFFILEFQDKTDIKTVKDCIYKIQSNWSNLSKRTDRPYSPFLLFHGTSDANLYELKNQLFNEDLIFTDGYPFKGSVFTPKMLI |
| | EGFSNKEIHFQFINDIDDFNETLNSINIRKEVYQFYTENCLDIPSQLPQVNIQVKDFADIKEIV* (SEQ ID NO: 346) |
| 46 | MSRNNDINAEVVSVSPNKLKISVDDLEEFKIAEEKLGVGSYLRVSDNQDVALLAIIDNFSIEVKESQKQKYMIEASPIGLVK |
| | NGKFYRGGDSLALPPKKVEPAKLDEIISIYSDSIDINDRFTFSSLSLNTKVSVPVNGNRFFNKHIAIVGSTGSGKSHTVAKILQ |
| | KAVDEKQEGYKGLNNSHIIIFDIHSEYENAFPNSNVLNVDTLTLPYWLLNGDELEELFLDTEANDHNQRNVFRQAITLNKKI |
| | HFQGDPATKEIISFHSPYYFDINEVINYINNRNNERKNKDNEHIWSDEEGNFKFDNENAHRLFKENVTPDGSSAGALNGKLL |
| | NFVDRLQSKIFDKRLDFILGEGSKSVTFKETLETLISYGKDKSNITILDVSGVPFEVLSICVSLISRLIFEFGYHSKKIKRKSNEN |
| | QDIPILIVYEEAHKYAPKSDLSKYRTSKEAIERIAKEGRKYGVTLLLASQRPSEISETIFSQCNTFISMRLTNPDDQNYVKRLL |
| | PDTVGDITNLLPSLKEGEALIMGDSISIPSIVKIEKCTIPPSSIDIKYLDEWRKEWVDSEFDKIIEQWSKS* (SEQ ID |
| | NO: 347) |
| 47 | MIMSTPWLTPIVADSDHAEANAVSYEALTPTELDSDKAGCYISALNYAYEHPDIRNIAVTGPYGAGKSSVLKTWCKAHNG |
| | TLRVLTVSLADFDMQRHVDESNGDSSSDEGTKNTGSVEKSIEYSILQQILYKNKKHELPCSRIDRISDVTAGQILRSASFLTG |
| | TILLSGAALFFLAPDYVTTKLSLPGAFARYLLECPFGVRVSGAVASVMGSLCLLLNQLHRIGIFDRKVSLDKVDLLKGAVTT |
| | RASSPSLLNVYIDEIVYFFDSTKYDVVIFEDLDRFNNGRIFVKLREINQIINNCLSDRKPVKFIYAVRDGIFNSAESRTKFFDFV |
| | MPVIPVMDNQNAYEHFVKKFKEEEINNNLSECISRIATFIPNMRVMHNITNEFRLYQNLVNSRENLAKLLAMIAYKNLCAE |
| | DYHGIDSKKGVLYHFIQSYLDHEIQNELLHSANNELEDMAQSLVAITNEKLANRENLREELLMPYLSKNYSGALVFYTEGR |
| | QISLDDLIQDEDEFLMLLDKENIQVVTPYNRQNFLMINQRDTEKLKQQYEKRCHLIETKSVDNITRVKNNISSLESLRTEILS |
| | GTVADIAEKMTNEGFVAWIKKKEDTGVLTIQSEHEQIDFIFFLLSSGYLSTDYMSYRSIFIPGGLSETDNLFLKDVMSGKGPE |
| | KTFSFHLDNVNNIVERLKKLGVLQRDNAQHPAVIRWLIDNDPDTLKNNIMALLSQTGSQRVVSLLMLMQNDFTTYVRLRY |
| | LEIFMSDEHILNRLLAHLCASEERTPEQKFFVQEIAAHLLCLTEKSNIWQSVEINKRIGELIDSSPILITAVPKGYGDAFFEVLK |
| | DNTLSVSYIPGDVGDEKCSVIRKIAGAGLFKYSVSNLKNVYLCLTQDKNEERMSFSLYPFHCLESLAISELTEILWTNIEDFIL |
| | SVFIESEEIDRIPELLNSSEVSMTVVEQIIAKMDFCINNLDDIINRSECADNNASGRNIYSMLLQHDRIFPSFDNIIHLLHDTSIN |
| | TSGELVQWVNEKHFEFEPSDIVINDTGIFNNFISELICSPVISEEALLKVLSNLNVVIIDVPENIPLRNAELLCSEKKLAPTVNV |
| | FTVLFNALSENVDDINRMNTLLGNLIAQRPEIITQEPEDIFYIEGDFDEELASELFRHKLIGMNIKVAALRWLRDNKPGILDKS |
| | YLLSLDILAELSPWMGDDDLRLTLLKRCLVAGDAGKDALCVVLNSFADESYHGLLPHDRFRKIPHSVDLWEVAELISNLGF |
| | IQPPKMGSGRDEHKIVITPVRYVRDVEFYD* (SEQ ID NO: 348) |
| 48 | MFLNDQETSTDLLYYTAIASTVVRLVDETSDAPITIGVHGDWGAGKSSVLKMLEAACEKKDKTHCIWFNGWTFEGFEDAK |
| | TVIIETIVEDLVASRPMSTKVAEAAKKVLRRIDWLKMAKKAGGLAFTAFTGIPTFDQIKGMYELASDFLSAPQDKLSAADF |
| | KAFAEKAGGFIKEADTDSNTLPKHIHAFREEFRALLDAAEIEKLVVIVDDLDRCLPKTAIETLEAIRLFLFVEKTAFVIGADE |
| | AMIEYAVKDHFPDLPQSTGPVSYARNYLEKLIQVPFRIPALGTAETRIYTTLLLAENALGSEDDNFKALLNKAREEMKRPWI |
| | SRGLDREAVMAALNGKIPEVVENALLFSLHVTPMLSSGTHGNPRQIKRFLNSMMLRQAIADERGFGSDIKRPVLAKIMLAE |
| | RFYPSVYGKLVQLVSNHPEGKPEALAEFEALVRGGKTAPKSRADSKENSSESEDVQNWLKIDWAIGWAKAEPALSGEDLR |
| | PYVFVTRDKHSTLSNLVVSSHLIPIMEKLLGPKIGMVKIKGDLEKLSPPDADELFEMLSDKLFQEDSFNRKPRGFDGLEYLV |
| | ETQPHLQRRLIDFARRIPVKKAGGWLATRIAQSLVDPTLIEEYTKLIQEWASQDENLSLSKSAKATLQLSGYQH* (SEQ ID |
| | NO: 349) |
| 49 | MGTSKAYGGPVHGLIPDFVENPSPPTLPPVDPADDSTLDTPLIPPDSSGSGPLSTPKANFTRYSRSGSRSSLGKAVAGYVRNG |
| | VGGAGRASRRMGASRAAAGGLLGLISDYQQGGATQALERFNLGNLAGQSASTALLSLVEFLCPPGGSVDEGVARQAMLE |
| | TIADMSDVGEENFDELTPDQLKEVFIGFVVHSIEGRLMADIGKNGIKLPDDIDAIVSIQEDLHDFVDGATRTQLREELRNLTG |
| | LSGDAIDRKVEEIYTVAFELLAREGERLE* (SEQ ID NO: 350) |
| 50 | MSHHTLVARLGTDDNSDLQLSRQSTHLTEINFLKENGKLDFGLGQALNGLSDLGLTPMDVSVDLALLAATVTAADTRISR |
| | GHNAQDLWTREIALYIPVASPTLWNSQTGLLSRMLNFLTGDRWTIHFRSRPVIEHGLIQRSSKERSVNPTSVCLFSGGLDSFI |
| | GAIDLLSNGGTPLLISHYWDTTTSVYQQKCAQLLSERYGQSFSHVRARVGFEKTTIEGEDGENTLRGRSFMFFSLATMAAD |
| | ALGGPVTINVPENGLISLNVPLDPLRVGALSTRTTHPFYMARFNELLGNLGISAHLENPYAYKTKGEMAIHCHDHAFLRQH |
| | AADTMSCSSPQSTRWNPALNEQQSTHCGRCVPCLIRRASLFTAFGTDDTIYRIPDLRSRVLDSSKPEGEHVRAFQFALARLA |
| | RSPSRAKFDIHKPGPLSDYPDCLAEYEGVYLRGMKEVERLLSGVITRPLT* (SEQ ID NO: 351) |
| 51 | MKLAGQKPAPQWVDFHCHLDLYPNHSALIRECDISRVATLAVTTTPKAWMRNRELTSDSPYVRVALGLHPQLIAEREHEI |
| | ALLEHYLPSARYVGEIGLDASPRFYRSFEAQERIFSRILNACFEQGDKILSIHSVRAAAKVLGHLENTRLTENCKAVLHWFT |
| | GSISEARRAVELGCYFSINEEMLRSPKHRKLVSFLPFERILTETDGPFVFHEEKAIHPRDVQRTVHEIAQIHHVSDTDAAMRIL |
| | YNLRSLVTNSSHSENSS* (SEQ ID NO: 352) |
| 52 | MSTVDTSTAEELNQGGSDFILTSLEAMRKKLLDLTSRNRLLNFPITQKGSSLRIVDELPEQLYETLCSEIPMEFAPVPDPTRA |
| | QLLEHGYLKVGPDGKDIQLRAHPSAKDWAHVLGIRTDFDLPDSHKTVVSDSDRELLEKAHQFILQYAQGQNGKLTGIRSE |
| | YVNQGIALSALKEACCLAGYEGLEDFERQAKAGNEISISSSNPSHDDNRIQALLYPNELEACLRAIYGKAQTALEESGANIL |
| | YLALGFLEWYESDSSEKARYAPLFTIPVRCERGKLDPKDGLYKFQLYYTGEDILPNLSLKEKLQADFGLALPLFNEEETPES |
| | YFASVKKVVEQHKPKWSVKRYGALSLLNFGKMMMYLDLDPARWPCDKRNILSHEVIRRFFTSQSCGQENSGLPGGFGQH |
| | EYCIDSYPDIHDKVPLIDDADSSQHSALIDAIRGQNLVIEGPPGSGKSQTITNLIAAALLNGKKVLFVAEKMAALEVVKRRL |
| | DRAGLGQFCLELHSHKTHKRKVLDDINARLVSQATMPTMEEIDAQILRYEDLKQQLNEYAALINNQWAQTGKTIHQILSG |
| | ATRYRHKLDIDATALHIENLSGKQLDKVTQLRLRDQIVEFSRIYKEVREQVGANAEIYEHPWSGVNNTQIQLFDSARIVDLL |
| | QTWQTSIIDFQHSYQEYVDKWALEGESLNTLQYIEQLVEDQSNLPVLCGSEHFPALSELDSPDAIARVRHYLDRFELLQGH |
| | YVALSQVIEPQKLRLLEQGQSCDFPREELEKYGAAEDFTLRDLVRWLESIQSIHDELSSIYAQLNDFKNALPDGIASYIDDSQ |
| | AGLLFCSELLSILGALPTELIRVRDPLFDDDDIDAVLRDLMCQIETLRPLRDGLSTLYQLDQLPSQEMLAHAVAVIQQGGLF |
| | AWFKSDWRSAKALLMAQSRKPDTKFAELKRCSADLLKYSELLQRFEQSDFGNQLGNAFRGLDTDCEQLMLLRDWYKKV |
| | RACYGIGFGKRVAIGSGLFNLDGEIIKGVHLIEKSQISSRLMTLVKRVEHEAKLLPRISSLLEEHASWLGEQGVLMQSYRQV |
| | RNTLIALQGWFINPDISLEQMTHSSEILQNINDLQISLENDSLQLGAFLQLTPLACGAYKNNQLTLDTINDTLNFAEQLVDKI |
| | NCVSLATQIRHLASGSDYDLLCRDGGEIVSKWNEQIKNAELYALETKLERSQWLKSTDGSLNTLIERNERAIQQPRWLNG |
| | WVNFIRCYEQMHENGLQRIWSAVLAGSLPIEKVELGLALAIHDQLAREVIHIHPELMRVSGSQRNALQKSFKEYDKKLIEL |
| | QRQRIAAKIACRNIPEGNSGGKKSEYTELALIKNELGKKTRHIPIRQLVNRACNALVAIKPCFMMGPMSAAHYLEPGRMEF |
| | DLVVMDEASQVKPEDALGVIARGKQLVVVGDPKQLPPTSFFDRSADGEDDDDAAALSDTDSILDAALPLFPMRRLRWHY |
| | RSRHEKLIAYSNRHFYNSDLVIFPSPNAESPEYGIKFTYVSKGRFSNQHNIEEAQAVAEAVLHHAHHRPGESLGVVAMSSK |
| | QRDQIERAIDELRRNRPEFNDAIDGLHAMEEPLFVKNLENVQGDERDVIFISFTYGPSEHGGKVYQRFGPINSDVGWRRLN |
| | VLFTRSKKRMHVFSSMRSEDVLTSETSKLGVISLKGFLQFAESGKLDSLTTHTGRAPDSDFEVAVMEALNHAGFECEPQVG |
| | VAGFFIDLAVKDPGCPGRYLMGIECDGAAYHSAKSARDRDRLRQEVLERLGWRISRIWSTDWFSNPDEVLSPIIRKLHELK |
| | TLAPDVVVPSYEYVETIESSAEVASDSIDSLMPNLGLKEQLKYFATHVIEVELPNVDADRRLLRPAMLEALLEHQPLSRSEF |
| | VERIPHYLRQATDVYEAQRFLDRVLALIDGAEAEANDAAFESELA* (SEQ ID NO: 353) |
| 53 | MHRTISEFYRIPPLLIRALKSGISSVVEFHLNRGLPKDSRDSLGNSPLMIAAQYGHFAICEMLLSAGVDVEHQNNLGLRASDL |
| | AQEQKLRDLLARYRQPLSLAELERSVVSVEDSETEAELPSAEIPMDFMLWDAEVELKPAEDNLTLRHASAEAQQLLSRYRP |
| | KDNSAEWSDIELTLPEPLTPVSHSPQNYPHLSTLLIGALDTGRISLRDIWHAGEEDFGMQWPEFRLSVEALIRDLPLIVDDDD |
| | IIPPDAAPATLSVSEPLEPWFDAFNALRQFGIVENYLVDIRQWDVVDKTKEERLGQRMDTALINLIRILAGLSEAEYMQLLQ |
| | PNYLPEPAPEISEEEDVAEEADEEMPPVSDDDDDNDDTISFIELLVLLRSGKAGEYQDNHIPRPEYADLQQIVERARTLIPDE |
| | GHKISLYVSSYREAWEGLIHANLRLVVTIANKYRGRGLDVEDLIQEGNLGLIKAVEKFDYRRGFKFSTYATWWIRQKISRA |
| | IADQAQLIRLPVHFYEQFRRWRNSRDQLLYRQGITPTIKRLQALTDLPENQLKRMAKYEEQTVLIGDFHDDAQDSEAALSG |
| | DAILTGKDFTSAPVQSLELRECVSLVLETLLPREKQIIKMRFGIGMTQDFTLEEVGKQFDVTRERIRQIEAKALRKLRYHSRA |
| | SKLGGFVEQWETALSEMQEEEE* (SEQ ID NO: 354) |
| 54 | MTTMRHAPPNAAIMIEALRGLGYNTATALADIIDNSISAGARKVDLTFHWRESDSYIVVRDNGCGMSAAELDVAMRLGV |
| | KNPLTKRSGHDLGRFGLGLKTASFSQCRRLTVASKKEEITTILRWDLDILAASTDDGWYLLEGADPGSQEALANEEPDSHG |
| | TVVLWDVLDRIVTPGYGEKDFLNLMDGVEQHLAMVFHRFLEGNAPRLTLTLNGRKIKAWDPFLSGHPSKPWHSPSAMAP |
| | GAPAVKVECHVLPHQDHLTTQEYQQAQGPAGWTAQQGFYVYRNERLLVAGNWLGLGSPRAWTKDETHRLARIRLDIPN |
| | DADIDWKIDIRKSMARPPVSLRPWLTQLAQSTRDRAVRTFAKRGKMNKRKPGEELVQLWQAQKTPSGVRYQISLQHPVIS |
| | NVLSQAGELSPQIQAMLRLIEETVPVQQIWLDTAETKETPRTGFETAPPAEVLSVLQVMYQTMVGQQAMSPALAKQHLQN |
| | MEPFDNYPELIALLPDDQHEKSL* (SEQ ID NO: 355) |
| 55 | MSLNPLDDTQLSVLQIVQTFLQSQDKSTITPGILRQHIDMVCQMKPEWSRLDSREILVEELIRRYSIWMGEDSSLSNDEGHQ |
| | PWLTADAKREWRYWHRYRQWLGKTMPWGVLDTLDRSTDRVLGLLEQPGREGRWDRRGLVVGHVQSGKTSHYTGLICK |
| | AADAGYKIIIVLAGLHNNLRSQTQMRLDEGFLGYETSPLREKVTIIGVGAIDSDPVIRPNYVTNRSEKGDFSAGVAKNLGISP |
| | EQRPWLFVVKKNKSILKRLHTWIENHVATSVDPITGKRFVSELPLLMIDDEADNASVDTGEIVYDDDGKPDAEHQPTAINS |
| | LIRKLLMQFSRKAYVGYTATPFANIFIHESNETRDEGPDLFPSAFIINLGAPSNYIGPARVFGRATAEGRSGEFPLIRRVSDHC |
| | SDDGKRGWMPVSHKSSHYPTLDTLTHFPDSLKHAIDSFLLACCVRELRGQGEKHSSMLVHVTRFNKVQSVVYENIDAYIQ |
| | DVRQRLTRRIGHEPFLHQLESLWQADFLPTNQAIREVMPQQVPDDAFEWQEIVDKLYTVIENVSVRMINGTAKDALDYSD |
| | SATGLKVIAIGGDKLARGLTLEGLCTSYFLRASRMYDTLMQMGRWFGYRQGYLDVCRLYTTDELIEWFEHIADASEELRE |
| | EFDNMVASGGTPRDFGLKVKSHPVLMVTSPLKMRSARSLWLSFSGTVVETISLFKEQEYHKRNYVAFQRLTGRVGAGAPI |
| | PERRRGDKIEKWNGVIWQNISPEPIIDFLTEYETHAQARKANSKLLADFVTRMNRVDELTQWTVAVIGGGIDRHHDVCGFS |
| | VPLMMRKASEGVTDRYSIGRLLSPRDEGIDCDESTWLAALEETQRIFHADPGRNEGREEPVVPGGVVLRRIKGFGINDIPAQ |
| | RQKGLLLIYLLDPQQALSAAEYQEDALPVVAFGISFPGSRSGVTVEYKVNNVLWEQEYGAAE* (SEQ ID NO: 356) |
| 56 | MVRLSKDDLLAAWKALDRSQIDELPGAQGWRGIRLFTHQGCSFHAGRRQPDNEEMLIAVFPHPLSPGSAALPSCKGFRVE |
| | MAGTEEGGQNGLMIRRQQTGNVDVFTTMILDILHSLLNVSKPRLFETLLRRIRLWQAFMERDTRPLSQEEEVGLIGELTCLE |
| | RLIESGLAPSTAVEAWIGPQHGLQDFALDERAIEIKSTTAAKGFCITIHSLEQLDWQRAGSLVLCGLRFSEHPTGATLNDIISR |
| | LRQRFEGNATAACIFEGSLCHVGYFTEHAEFYTRHFLLTEAFALPIEADFPSLTHANVPLPVVSARYQLELQTLIPQAQDFN |
| | HCLSDFAGLPHGNY* (SEQ ID NO: 357) |
| 57 | MEIIDFLRQTQNEIRKEYQDQMAQPGVESPFPELIFTDIVMRHMADIGMTFDDAETCHFMAKVSGHNVRLSGYAFSEDGDQ |
| | LDLFVSIYHGSDELCHVPDAETKAIAGHCIQFLQKCVDGKLSSTLDQSNDAWQLVTTIEQSYAELEQIRIYVLTDGQVKTR |
| | WYQSRDVAGKTIKLEVMDIVRLFNHWQEGKPRDELQVNFDEVAGGALPCVWIPDEMGEYDYALTVVPGETLRFIYEKYG |
| | NRILEANVRSFLSQTGKVNKGIRDTLREQPERFMAYNNGIVIVADQVRLGEAPGGGPGIAWMQGMQIVNGGQTTASMFFT |
| | KKKFPATNLRNVRVPAKVIVLKQTNNAQEEMLIADISRFSNSQNKVNISDLSANRPVHVQLEKMANTVYCPDGYSRWFYE |
| | RANGSYKVMLEREGKTPAGIKRLKDAIPPSRRITKTDFAKYHCAWLQRPDLVSLGGQKNFAALMTMIDKDTERYGDELNI |
| | ETFKNYIAQAIIYKKAYKLINSLFPAFKANIAAYTVAAYSHLYGNKTDLAEIWNQQGIEETMGNRLVSLAHRVNSLLTESA |
| | NGRMISEWAKKPECWDYVRSKIYFSAQGKKDDFSHGEIA* (SEQ ID NO: 358) |
| 58 | MAYEAQISRTNPAAFLFVVDQSGSMSDKMSSGRSKAEFVADALNRTLMNLITRCTKSEGVRDYFEIGVLGYGGQGVSNGF |
| | SGSLGGQVLNPISALEQNPARVEDRKRKMDDGAGGIIETAIKFPVWFDPIASGGTPMREALTRAAEELVTWCDAHPDCYPP |
| | TILHVTDGESNDGDPEEIANHLRQIRTNDGEVLILNIHVSSLGNDPIRFPSSDTGLPDAYAKLLFRMSSPLPEHLVRFAQEKG |
| | HTVGIESRGFMFNAEAAELVDFFDIGTRASQLR* (SEQ ID NO: 359) |
| 59 | MKLEFLGTVPKDPEYPKANEDKFAFSEDGRRLALCDGASESFNSKLWADLLARKFTADPKVNPEWVASALAEYSATHDFP |
| | SMSWSQQAAFERGSFATLIGVEEFEEHQAVEILAIGDSITMLVDCGKLICAWPFDNPEKFNERPTLLATLYAHNNFVGGSTF |
| | WTRHGKTFYLEKLTQPKLLCMTDALGEWALKQALAEDSGFIELLSLQTEEELAELVLRERAAKRMHIDDSTLLVLSF* |
| | (SEQ ID NO: 360) |
| 60 | MPYPSLEQYNQAFQLHSKLLIDPELKSGTVATTGLGLPLAISGGFALTYTIKSGAKKYAVRCFHRESKALERRYEAISRKISS |
| | LRSPYFLDFQFQPQGVKVEGISYPIVKMAWAKGETLGEFLEVNRRSAQAIAKLSASIESLAAYLEKEKIAHGDFQTGNLMV |
| | SDGGATVQLIDYDGMFVDEIKTLGSSELGHVNFQHPRRKATNPFNHTLDRFSLISLWLALKALQIDPSIWDKSNSELDAIIFR |
| | ANDFVDPGSSSILGMLSGIQQLSTHVKNFAAVCASAMEKTPSLGDFIASKNIPISLASISMNGDIPVSRLKPGYIGAYTVLSAL |
| | DYSACLQRVGDKVEVIGKIIDVKLNKTRNGKPYIFVNFGDWRGNIFKISIWSEGISALPSKPDASWIGKWISVIGLMEPPYVS |
| | GKYKYSHISITVTTIGQMTVLSEPDARWRLAGPNESRQTLTSTSSNQEALERIKSKSTTSTPMPMNTNATTANQAILNKLRA |
| | STQTVAAARAQTQHVVPNKSSTHYVAPTGTSASQPVQNIPSPASTSKQQTSQKNIVTKILKWLFG* (SEQ ID NO: 361) |
| 61 | MNEHLSHMDVHTLFEEMDEQADGITFKYSFDDIAKSNALVVTEFVNFERDSTVALLASLLTLPAHQSQCLRFELLTSLALIH |
| | CKGQQIANIDDVKRWYVTIGESSSIVGEDPAEDVFVALVDNKKGDYRVLEGVWEAAGFYTQLMVEIVSDMPDTHRYRSL |
| | KLAIQAILRLSDVICARSGLYRFQEGADEFPDSLDTAGLDEKTLCSRVTLSERSLRAEGIKLADLAPFILEPSHISMLGNQVPG |
| | EGMLEQRPLLRTRDGIVVVLPTAMTIALRQAVITFAKRTEELSELDKALANVYSLTFSEMPVFGNGGRLRRLTWEKYKMS |
| | RTTMVTSIVDAGHLMVLQFVLPSIQQYADTGFNNLLQLDEETTQFLDNSVEQITVDLAKQPGFQRGIVVRIACGWGAGFM |
| | GVPPQLPDGWGFEWMSGADFVRFGALPDMSPIAFWRVQDAVETIRQAGVRLINMSGTLNLLGWIRANDGHMVPHDQLP |
| | DDRITPEHPLMLMIPTNLLRGIRIAADTGYDRHRISDNNGKWHRVMRPSAEDFFPTERQSKCYASIDDLEAQRLTCVYEGQ |
| | GNLWVTLEAPEMEDWMLLVELAKMVRTWIGRIGEALEVLSEQPIKKSLKVYLHFDGNDNIGRFDGENFSDDMNTFWRLE |
| | RIHEHGAIRVVLQDGYLAGFRLPDNRAERALVRALGTAFATLLRMKEPVDKGVTVEQIAVPNDRARSFHIMQAYDFNQYL |
| | GRSLTKRLLAIEDIDSAAARIELAWRAVSTDAPSRYQGKKEVGKLLNDVVDVLIQDLLSELSRFDRKQTVMRLLENVVKA |
| | RCEEAHWRSTAAAVLGLHAGEEGVEETIAQEMSRYAGAALTSRLIIELAICVCPTSGGIEPSDMALSKLLARASLLFRIGGM |
| | SDAVRFGALPADIRISPLGDLLFRDELGKMVLEPMLSKVTNERFEEQAAQFEQHYVKTAGGDDENSKQDSVAAETTEDQT |
| | DIFLAFWKAEMGFTLEDGMRFIQFLESIGIEQESAIFEMRRSQLADAAKSAGLADETIDAFLNQFILSARPKWDVVPDGFDL |
| | SDIYPWRFGRRLSVAVRPLLQIEESHDPLIVIAPGLLNLSLKYVFDGAYTGQFKRDFFRTEGMRDTWLGGAREGHTFEKTLE |
| | RELREIGWTVRRGIGFPEILRRNLPGDPGDIDLLAWRSDRNQVLVIECKDLSLARNYSEVASQLSEYQGDDIKGKPDKLKK |
| | HLKRVLLAKENIDNFAKFTSIANPEIVSWLVFSGASPIAYAQSKIEALAGTNVGRPSDLLNF* (SEQ ID NO: 362) |
| 62 | MVGSRWYKFDFHNHTPASHDYKIPDISPREWLLAYMKQHVDCVVISDHNSGAWVDVLKGELENMSRDASTGDLPEFRPL |
| | TLFPGVELTATGNVHILAVLHTHSTSADVERLLAQCNNNSPIPSEVPNHQLVLQLGPAGIISNIRRNPKAVCILAHIDAAKGV |
| | LSLTNQAELTAAFQESPHAVEIRHRVEDITDGTRRRLIDNLPWLRGSDAHHPEQAGVRTCWLKMSSPDFDGLRHALLDPEN |
| | CVLFDQLPPEEPASYLRSLKFRTRHCHPVGQDSASVEFSPFYNAVIGSRGSGKSTLIESIRLAMRKTEGLTATQGSKLDQFIR |
| | TGMEADSFIECIFHKEGTDFRLSWRPDSKHELHIFSDGEWMPDSHWSADRFPLSIYSQKMLYELASDTGAFLRVCDESPVV |
| | NKRAWKERWDQLEREYLNEQITLRGLRARQGSADSLRGELSDAERAVSQLQSSAYYPVCRQLALARNELSAATLPLEHFE |
| | RRIAAIQALAEEPLQRSDIPPEPSGLLMAFMARLSSVQQQYDQRLNTLLAEYAAELAGIRREQSFIALRTAVSDQETNVESE |
| | AVSLRARGLNPDVLNELMARCESLKNELRNYDGLDGAISASVARSEQLLAEMRAHRMALTDNRKAFLSSLSLSALEIKILP |
| | LCAPYEDVISGYQTVTGISNFAERIYDNSDGSGLLSDFISERPFSPLPAATEKKYRALDELKALHHSIRLDNSEAGAGLHGSF |
| | RNRLRSLNDQQLDALQCWYPDDGIHIRYQTPGGQMEDIAFASPGQKGASMLQFLLSYGTDPLLLDQPEDDLDCLMLSMSV |
| | IPAIMSNKKRRQLIIVSHSAPIVVNGDAEYVISMQHDRTGLYPGLCGALQEAPMKALICRQMEGGEKAFRSRYERILS* |
| | (SEQ ID NO: 363) |
| 63 | MDYLSEVLKIIEGATKANASMASNYAGLLADKLEQKGEVKQARMIRERLLRAPQALAGAQRAGGGISLGSLPVDIDSRLN |
| | TVDVSYPKLDSSEIFLPAAISTRVEEFITNVQRYDEFVKADAALPSRMLVYGKPGTGKTMLSKYIATRLDFPLLTVRCDTLIS |
| | SLLGQTSKNLRQVFDYVMQRPSVLFLDEFDALAGARGNERDIGELQRVVISLLQNMDAASEDTVIIASTNHEQLLDPAIWR |
| | RFSFRIPMPLPDIHQRELIWKNRLKNMICSDLDLSDLSRKSEGLSGAIIEQVSLDARRDAVIEGASVINHHKLYRRLYLAQSL |
| | MEGVNLSTYEDEIRWLRSKDKKLFSIRVLANLYKLTSRVISNILKESGAYEQKGYTV* (SEQ ID NO: 364) |
| 64 | MSRRGTQFSNAKVTNPMLRIPFSSSDLGAIVNAGGGAKVLVDVTAEYRQGLVRNLTTSKHYLESKLSEYPGSLGTLVFKLR |
| | DQGIAKTHRPNKIAQEAGLQNAGHAKIDEMLVAAHAGCFDVLESVILHRNIKAILANLSAIERIEPWDENRKVPGGTDGLF |
| | ESSNILVRLFEYTGEDATYNNYENVISILEQHGVKYDEIRQKCGLPLLRIMDLSPNDRYILDILIDYPGIRTLIPEPKYSAFPVS |
| | VSDSVGIETNSFPVPSEELPIVAVFDTGVSPIAATITPWVVSRETYVIPPDTSYEHGTMVSSLISGAHFLNDNHPWIPDTKSKI |
| | HDVCALDENGSYISDLILRLADAVNKRPDIKVWNLSLGGGPCNEQTFSDFAMELDRLSDKFGILFVVAAGNYVDEPIRTWP |
| | NPDPLGGADLISSPGESVRALTVGSVSHMEANDALSEIGTPTPYTRRGPGPVFTPKPDIIHAGGGVHRPWNVGASSLKVVGP |
| | DNRLCSNFGTSFAAPIVASLAAHTWQRIATNTDFNVSPSLIKALLIHSAQLSSPDYSPSERRYLGAGIPNEVIETLYDSDDRFT |
| | LIFQTFLVPGVRWRKDNYPIPSALIQNGKFKGEIVITAAYAPPLNPNAGSEYVRANVELSFGLIENNTIKGKVPMEGENGQS |
| | GYERAQIEHGGKWSPVKIHRKAFNKGITSGNWALQAKTTLRANEPALMEPLPVTIVVTLKSLDGNTQVYADGVRALNAN |
| | NWAHYPLPARVPVSV* (SEQ ID NO: 365) |
| 65 | MKTVRSACQLQPKALEINVGDQIEQLDQIINDTNGQEYFKKTFITDGFKTLLSKGMARLAGKSNDTVFHLKQAMGGGKTH |
| | LMVGFGLLAKDAALRNSHLGSMPYQSDFGSAKIAAFNGRNNPHSYFWGEIARQLGREGVFREYWESGAKAPDEQAWINI |
| | FDGEEPILILLDEMPPYFHYYSTQVLGQGTIADVVTRAFSNMLTAAQKKKNVCIVVSDLEAAYDTGGKLIQRALDDATQEL |
| | GRAEVSITPVNLESNEIYEILRKRLFLSLPDKNEVSEIASIYASRLAEAAKAKTVERSAEALANDIESTYPFHPSFKSIVALFKE |
| | NEKFKQTRGLMELVSRLLKSVWESDEEVYLIGAQHFDLSIHDVREKLAEISEMRDVIARDLWDSTDSAHAQIIDLNNGNHY |
| | AQQVGTLLLTASLSTAVNSVKGLTESEMLECLIDPNHQGSDYRNAFTELAKSAWYLHQTQEGRNYFSHQENLTKKLQGY |
| | ADKAPQNKVDELIRHRLEEMYRPVTKEAYEKVLPLPEMDEAQATLRSGRALLIISPDGKTPPGVVGNFFKGLVNKNNILVL |
| | TGDKSSIASIEKAARHVYAVTKADNEITASHPQRKELDEKKAQYEQDFQTTVLSVFDKLLFPGNNRGEDVLRPKALDSTYP |
| | SNEPYNGERQVVKTLTSDPIKLYTQINENFDALRARAESLLFGTLDEARKTDLLDKMKQKTQMPWLPSRGFDQLAIEAYQ |
| | RGVWEDLGNGYITKKPKPKTTEVIISEDSSPDDAGTVRLKIGVANAGNSPRIHYAEDDEVTESSPVLSDNTLATKALRVQFL |
| | AVDPTGKNLTGNPTTWKNRLTLRNRFDEVARTVELFVAPRGTIKYTLDGSEARNGETYTVPIQLADQEATIYVFAECDGLE |
| | EKRNFTFAAAGSKEIPIIKDKPATLVSPSPKRMDSSAKTYEGLKIAKEKGIEFEQISLMVGSAPKVIHISLGEMKISAEFIETVL |
| | THLQTVLSPEAPVVMTFKKAYTQTGHDLEQFVKQLGIEIGNGEVEQR* (SEQ ID NO: 366) |
| 66 | MNKTVDFGAPSEFGMHHFYVEIPAAPRDAVVIYEDYGFDGEDSRRETVECRLILARELWTKIRDDVRRDFNARLKIKKQSS |
| | GTWSTGKVKLDRFLGRELCVLGWAAEHASPDECLVICQKWLALRPEERWWLYSKTAAEAGRDDQTQRGWRKALYCAL |
| | SDGANIKLETKKKPKSKKLQVEDETQDLFGFMEKGEF* (SEQ ID NO: 367) |
| 67 | MALQPFEWRDKPSLIEHLFPVQKISAETFKERMASHGQLLVSLGAFWKGRKPLILNKACILGSLLPATDNPLEDLEVFELLM |
| | GIDSESMQKRIEASLPASKQETIGDYLVLPYAEQIRIAKRPEEIDESLFVHIWNRVNNHLGTSAHTFAQLVEELGVARFGHRP |
| | RVADVFSGSGQIPFEAARLGCDVYASDLNPISCMLTWGALNVVGASAQKRVEIDKAQRDIVKKVQKEIDELDIESDGRGW |
| | RAKVFLYCVEVTCPESGWRVPLIPSLIISNSFRVVAELKPVPAERRYDISIREVSTDEELEFYKSGTIQDGEVIHSPDGKTQYR |
| | VNIKTIRGDYKEGKENLNKLRMWEKTDFAPRPDDIFQDRLFCVQWMKKKPKGSQYYYEFRTVTNDDLKREKKVIEHVAS |
| | KLDDWQKQGLVPDMVIEAGDKTDEPIRTRGWTHWHHLFHPRQLLFLSLVNKYSLAEGKFNFLQCMNHLSKLTRWRPQA |
| | GGGGGSAATFDNQALNTLYNYPVRATGSIENILAAQHNHCGISENVSFVVNSHPAPELDVENDIYITDPPYGDAVKYEEITE |
| | FFIAWLRKNPPKEFAHWTWDSRRSLAVKGEDEGFRTGMVAAYRKMAQKMPDNGLQVLMFTHQSGAIWADMANIIWAS |
| | GLQVTAAWYVVTETDSALRGGSNVKGTIILILRKRHQALETFRDDLGWEIEEAVKEQVESLIGLDKKVRSQGAEGLYTDA |
| | DLQMAGYAAALKVLTAYSRIDGKDMVTEAEAPRQKGKKTFVDELIDFAVQTAVQFLVPVGFEKSEWQKLQAVERFYLK |
| | MAEMEHQGAKTLDNYQNFAKAFKVHHFDQLMSDASKANSARLKLSTEFRSTMMSGDAEMTGTPLRALLYALFEISKEVE |
| | VDDVLLHLMENCPNYLPNKQLLAKMADYLAEKREGLKGTKTFNPEQEASSARVLAEAIRNQRL* (SEQ ID NO: 368) |
| 68 | MAIKRFSSRTERLDTEFLAESLKGAAKYFRIAGYFRSSIFELVGEEIAKIPEVKIICNSELDLADFQVATGRNTALKERWNEV |
| | DVEAEALLKKERYQILDQLLHSGNVEIRVVPRERLFLHGKAGSIHYADGSRKSFIGSVNESKSAFAHNYELVWQDDDEESA |
| | DWVEREFWALWTEGVPLPDAILAEIHRVSNRREVTVDVLKPEEVPAAAMAEAPIYRGGEQLQPWQRSFVTMFLEHREIYG |
| | KARLLLADEVGVGKTLSMATSALVSALLDDGPVLILAPSTLTIQWQIEMMDKLGVPAAVWSSQKKVWLGVEGQILSPRG |
| | DASSIKKCPYRIAIISTGLIMHQREKTDFVKEAGMLLKNRFGTVILDEAHKARIRGGLGDQASEPNNLMAFMLQIGRRTRHL |
| | VLGTATPIQTNVRELWDLLGILNSGAEFVLGDALSPWHDHEQAIPLITGQTQVTSEAEVWHWLSNPLPPSNEHHTVQQIRD |
| | YLSIDNKSFGYSHRFEDLDYMIQSLWLSECMTPSFFKENNPILRHTVLRKRKQLEDDGLLERVGVNTHPIKRNLAQYQSRF |
| | VGLGIPTNTPFQVAYEKAEEFSKLLQSRTRAAGFMKSLMLQRICSSFASGLKTAQKMLKHTVSDEDEDLVEDVEHLLSEMT |
| | PAEVACLREIETQLSRPEAVDSKLNTVKWFLTEFRTDGKTWLEHGCIIFSQYYDTAEWIAKELAKSLKGEVVAVYAGVGK |
| | SGLFRGEQFNNVERELIKSAVKTREILLVVATDAACEGLNLQTLGTLINVDLPWNPSRLEQRLGRIKRFGQTRKFVDMLNL |
| | VYSETQDEKVYNVLSERLRDTYDIFGSLPDTIDDEWIDNEEELNTRMDEYMHERKKAQDAFSVKYRGTLDPDAHLWERC |
| | ATVLSRRDIVSKLSEPWGS* (SEQ ID NO: 369) |

TABLE 16A
Additional tested homologs of predicted defense systems

| System | | Observed | # | Source | | Pro- | | | |
|---|---|---|---|---|---|---|---|---|---|
| # | Name | Activity | Genes | Organism | Strain | moter | Codon | Gene A | Gene B |
| 1 | Retron-TIR | + | 1 | Escherichia coli | NCTC9024 | Native | Native | STF89551.1 | — |
| 2 | Retron-TOPRIM | − | 1 | Escherichia coli | NCTC13441 | Native | Native | WP_000476153.1 | — |
| 5 | RT-nitrilase | − | 1 | Escherichia coli | N1 | Native | Human | WP_001121606.1 | — |
| | (UG1) | | | | | | | | |
| 7 | RT (UG3) + RT | − | 2 | Escherichia coli | NCTC9091 | Native | Native | STJ76581.1 | STJ76580.1 |
| | (UG8) | | | | | | | | |
| 7 | RT (UG3) + RT | − | 2 | Salmonella | NCTC6026 | Native | Native | WP_001530977.1 | WP_001185451.1 |
| | (UG8) | | | enterica | | | | | |
| 7 | RT (UG3) + RT | − | 3 | Acinetobacter | NCTC7412 | Native | Native | WP_000227776.1 | WP_000620968.1 |
| | (UG8) | | | calcoaceticus | | | | | |
| 8 | RT (UG15) | + | 1 | Escherichia coli | STEC66 | Native | Human | WP_032207424.1 | — |
| 10 | ATPase + | + | 2 | Escherichia coli | NCTC11116 | Native | Native | WP_096949333.1 | WP_001538182.1 |
| | adenosine | | | | | | | | |
| | deaminase | | | | | | | | |
| | (RADAR) | | | | | | | | |
| 13 | STAND | − | 1 | Escherichia coli | NCTC10650 | Native | Native | SQB54359.1 | — |
| 21 | Transmembrane | + | 1 | Escherichia coli | NCTC8620 | Native | Native | WP_048228060.1 | — |
| | ATPase | | | | | | | | |
| 22 | ATPase + QueC + | + | 4 | Escherichia coli | ECOR10 | Native | Native | WP_000269401.1 | WP_000537316.1 |
| | TatD DNAse | | | | | | | | |
| 23 | DUF4011- | − | 1 | Citrobacter | NCTC9067 | Native | Native | WP_115191085.1 | — |
| | helicase-Vsr- | | | braakii | | | | | |
| | DUF3320 | | | | | | | | |
| 28 | ATPase + | + | 2 | Escherichia coli | ECOR12 | Native | Native | OWD36540.1 | OWD36541.1 |
| | protease (ietAS) | | | | | | | | |
| 28 | ATPase + | − | 2 | Escherichia coli | NCTC9008 | Native | Native | WP_001460375.1 | WP_020244573.1 |
| | protease (ietAS) | | | | | | | | |
| 30 | Retron-protease | − | 1 | Proteus | 127_PMIR | Native | Native | WP_161800346.1 | — |
| | | | | mirabilis | | | | | |
| 30 | Retron-protease | − | 1 | Yersinia | 404/81 | Native | Native | WP_054888011.1 | — |
| | | | | aleksiciae | | | | | |
| 30 | Retron-protease | − | 1 | Yersinia | 3016/84 | Native | Native | WP_054872116.1 | — |
| | | | | bercovieri | | | | | |
| 30 | Retron-protease | − | 1 | Yersinia | ST5081 | Native | Native | WP_050337179.1 | — |
| | | | | enterocolitica | | | | | |
| 31 | RT-nitrilase | − | 1 | Escherichia coli | NCTC4169 | Native | Native | WP_001521910.1 | — |
| | (UG5) | | | | | | | | |
| 31 | RT-nitrilase | − | 1 | Klebsiella | KPNIH39 | Native | Native | WP_023301376.1 | — |
| | (UG5) | | | pneumoniae | | | | | |
| 32 | TOPRIM-RT- | − | 1 | Pseudomonas | DSM16299 | bla | Native | WP_084139843.1 | — |
| | nitrilase (UG10) | | | rhizosphaerae | | | | | |
| 32 | TOPRIM-RT- | − | 1 | Vogesella | DSM3303 | bla | Native | WP_120809745.1 | — |
| | nitrilase (UG10) | | | indigofera | | | | | |
| 33 | RT (UG7) | − | 1 | Escherichia coli | NCTC9069 | bla | Native | WP_000064054.1 | — |
| 34 | RT (UG9) + PolA | − | 2 | Photorhabdus sp. | CRCIA-P01 | lac | Native | WP_118986603.1 | WP_118986604.1 |
| 34 | RT (UG9) + PolA | − | 2 | Pantoea sp. | B40 | lac | Native | WP_042677494.1 | WP_128574327.1 |
| 34 | RT (UG9) + PolA | − | 2 | Vibrio | DSM17657 | lac | Native | WP_051241322.1 | WP_083962817.1 |
| | | | | litoralis | | | | | |
| 34 | RT (UG9) + PolA | − | 2 | Pseudomonas | Wood1 | lac | Native | WP_080587824.1 | WP_027911782.1 |
| | | | | brassicacearum | | | | | |
| 35 | DUF4297- | − | 1 | Escherichia coli | NCTC9036 | Native | Native | WP_060615938.1 | — |
| | STAND | | | | | | | | |
| 36 | DUF4297- | − | 1 | Salmonella | NCTC10718 | Native | Native | WP_115407481.1 | — |
| | STAND | | | enterica | | | | | |
| 37 | ATPase_GHKL + | − | 2 | Pectobacterium | CFBP3304 | bla | Native | WP_005974598.1 | WP_005974600.1 |
| | Helicase_SF2 | | | wasabiae | | | | | |
| 37 | ATPase_GHKL + | − | 2 | Vibrio | ATCC43516 | bla | Native | WP_061066216.1 | WP_061066217.1 |
| | Helicase_SF2 | | | harveyi | | | | | |
| 38 | ATPase_GHKL- | − | 1 | Raoultella | NCTC9528 | Native | Native | WP_112150151.1 | — |
| | DUF3684- | | | planticola | | | | | |
| | DUF3883 | | | | | | | | |
| 39 | TerY-P + helicase + | − | 7 | Obesumbacterium | DSM2777 | Native | Native | WP_057631338.1 | WP_057631339.1 |
| | HEPN + | | | proteus | | | | | |
| | ATPase + | | | | | | | | |
| | DUF2357 | | | | | | | | |
| 40 | Kinase-helicase | − | 2 | Escherichia coli | NCTC13919 | Native | Native | WP_000877066.1 | WP_001294844.1 |
| 41 | Helicase-DUF559 + | − | 5 | Plasticicumulans | DSM25287 | Native | Native | WP_132537919.1 | WP_132537920.1 |
| | SMC + McrB + | | | lactativorans | | | | | |
| | DUF2357 + | | | | | | | | |
| | ATPase | | | | | | | | |
| 41 | Helicase-DUF559 + | − | 5 | Yoonia | DSM29955 | bla | Native | PUB10544.1 | PUB10545.1 |
| | SMC + McrB + | | | sediminilitoris | | | | | |
| | DUF2357 + | | | | | | | | |
| | ATPase | | | | | | | | |
| 42 | GTPase + | − | 3 | Pantoea | DSM3873 | Native | Native | WP_084873987.1 | WP_084873988.1 |
| | GTPase + TM | | | cypripedii | | | | | |
| 43 | TM + GTPase + | − | 3 | Escherichia coli | NCTC10962 | Native | Native | STI27515.1 | STI27516.1 |
| | GTPase | | | | | | | | |
| 44 | Dcm + HerA + | − | 5 | Pseudomonas | NCTC10727 | Native | Native | WP_031690635.1 | WP_004363346.1 |
| | Vsr | | | aeruginosa | | | | | |
| 44 | Dem + HerA + | − | 5 | Aquimonas | DSM16957 | Native | Native | SDD97145.1 | SDD97170.1 |
| | Vsr | | | voraii | | | | | |
| 45 | RecQ | − | 1 | Klebsiella | NCTC11696 | Native | Native | WP_032728854.1 | — |
| | | | | oxytoca | | | | | |
| 46 | Histidine kinase + | − | 2 | Pseudomonas | NCTC13717 | Native | Native | WP_003450792.1 | WP_003450790.1 |
| | phosphoribosyltrans- | | | aeruginosa | | | | | |
| | ferase | | | | | | | | |
| 47 | PH-TerB- | − | 2 | Klebsiella | NCTC11357 | Native | Native | WP_126494466.1 | WP_023316678.1 |
| | DUF726 + TM | | | pneumoniae | | | | | |
| 48 | TerB + DUF2791 + | − | 3 | Escherichia coli | NCTC9024 | Native | Native | VDY98671.1 | VDY98669.1 |
| | Lhr helicase | | | | | | | | |

| System | | | | | | |
|---|---|---|---|---|---|---|
| # | Gene C | Gene D | Gene E | Gene F | Gene G | bp |
| 1 | — | — | — | — | — | 2393 |
| 2 | — | — | — | — | — | 2569 |
| 5 | — | — | — | — | — | 4154 |
| 7 | — | — | — | — | — | 3648 |
| 7 | — | — | — | — | — | 3818 |
| 7 | WP_000837118.1 | — | — | — | — | 4236 |
| 8 | — | — | — | — | — | 1951 |
| 10 | — | — | — | — | — | 5533 |
| 13 | — | — | — | — | — | 4781 |
| 21 | — | — | — | — | — | 4037 |
| 22 | WP_000192874.1 | WP_000020778.1 | — | — | — | 4891 |
| 23 | — | — | — | — | — | 6502 |
| 28 | — | — | — | — | — | 3678 |
| 28 | — | — | — | — | — | 3917 |
| 30 | — | — | — | — | — | 2009 |
| 30 | — | — | — | — | — | 1946 |
| 30 | — | — | — | — | — | 2032 |
| 30 | — | — | — | — | — | 1996 |
| 31 | — | — | — | — | — | 3679 |
| 31 | — | — | — | — | — | 3479 |
| 32 | — | — | — | — | — | 7494 |
| 32 | — | — | — | — | — | 7656 |
| 33 | — | — | — | — | — | 3894 |
| 34 | — | — | — | — | — | 3208 |
| 34 | — | — | — | — | — | 3211 |
| 34 | — | — | — | — | — | 3196 |
| 34 | — | — | — | — | — | 3382 |
| 35 | — | — | — | — | — | 6514 |
| 36 | — | — | — | — | — | 6261 |
| 37 | — | — | — | — | — | 10166 |
| 37 | — | — | — | — | — | 10210 |
| 38 | — | — | — | — | — | 5918 |
| 39 | WP_057631340.1 | WP_057631341.1 | WP_057631342.1 | WP_057631343.1 | WP_080376085.1 | 12191 |
| 40 | — | — | — | — | — | 6873 |
| 41 | WP_132537921.1 | WP_132537922.1 | WP_132537923.1 | — | — | 11931 |
| 41 | PUB10546.1 | PUB10547.1 | PUB10548.1 | — | — | 11041 |
| 42 | WP_084873989.1 | — | — | — | — | 4789 |
| 43 | STI27517.1 | — | — | — | — | 4577 |
| 44 | WP_004363343.1 | WP_003131012.1 | WP_071534163.1 | — | — | 11911 |
| 44 | SDD97192.1 | SDD97211.1 | SDD97232.1 | — | — | 11635 |
| 45 | — | — | — | — | — | 5424 |
| 46 | — | — | — | — | — | 4088 |
| 47 | — | — | — | — | — | 3637 |
| 48 | VDY98667.1 | — | — | — | — | 6037 |

TABLE 16B
(cloned sequences of systems #1-48)

| System | | |
|---|---|---|
| # | Name | Cloned Sequence |
| 1 | Retron- | atccctgaattccccgaaggtgaacaatccactgttcacccttcaccgtatattaacccgttatcacactgaaattaaaagagaaaaatgaaaggtgaacagtgtgaacaatca |
| | TIR | aatcaaaaaaactttctactcccactatagcctgactggtcgtctccaaaacgagcggaaaagcatcaacaatgaatagttaactgttaactccgcgccaactcattaccactta |
| | | actcaatgatattaaatggaaaactatcgaaatgaatactctgcaaaattaaatgcaaaaaaatatatgccagtcaaatttcgttacgcactctcttccaagaaagagataaatgc |
| | | tttatacgtccaccatactatgttatttttttaatacggctctgccttaaatctgtgaggttgtttcgcctcgaagtatcttatgttagcacatcacgctaccaatcagcggttagttactt |
| | | gacgtaactgttaattggctaaagtttgcatagagtgattgggcggagccgtaaatttagtccataaatacagtaacgaggtagagagtgtctttacatgacaagctactgatgc |
| | | ttagtctcaattcggcgaataaagaagaagatgagacaatcccggagttacctaagttagagcctcagccctatcaagctggaaataagttgaaatgggataataaagagctg |
| | | aaaaatcagcccatcacttcaaagaatgacattaatgtaatatgcaaaaaaattgaaaacaaaagcattgtaattacatcagcaaacgatgtagccaatctgttagaagtcccg |
| | | gtcggacaattattatttattttatataataaaaaagataactatagaacttttgaaataaaaaagaaaaatggaaaaagtagaatcataaatgcacctcaaggcggtttatcaattc |
| | | tgcaagagaaattaaagccagttcttgagtacttttatcgccccaaaaaaccagcacatggatttattaaggataaaagtatattaacaaatgcagaaaaacatacaaagaaaa |
| | | aatatgttgttaatgtagatttagaaaattattttggttcagtcactttcgctagagtatatgggatatttaaaagtaagccatttaatttctctcatcctgcggcgagtatattagctca |
| | | actatgtactaaggatggaaaattacctcaaggagcatgtacctcccctgttctagcaaatttagcatcagcctcactcgataaacacctaacccaactggcacgtagaaaaaa |
| | | catcacatatacaagatatgcagatgatattactttttcatttaatcaacgacaagtcagagaaatcataacgctagataatgaaaataattttgaattgggcgaggcgattatctct |
| | | gtgatagagaaaagtggcttcagcataaacacaagtaaattcagagttcagaaaagaaatgaacgtcaaaaagttactggtctagtggtaaatgaaaaagtaaatgttgagcg |
| | | taaatatcttagagttactcgttcattagttcataaatggagagaagacaagttaacatcagcattgttgtttgttactaaaaaaggttttaaggcaacaaataacgaacatgctata |
| | | tcaatttttcgcaatcatatttatgggcgattgagttttataaaaatgatccgtggtgaggacttcccgttatatcttaaattaatggctgaaatgagtcatcatgatcctttaaaaaca |
| | | aaagaagggcttagagcaatgaaagaaactgaaacttacgatgtatttatttgtcatgcaagcgaagataaaacatccatcgcaattccaatttacgaagaattaattaaattaa |
| | | atatatcaacattcatagatcatgttgaaataaattggggcgattcattaatccaaaaaattaactcagctcttgtaaagtctaaatatgtaattgccattctttcggctaattctgtag |
| | | ataaacattggcctaagaaagaattgcattctgtgcttgcaagagaaatcactgaaggtgaagtaaaattacttactcttgtaaaagaagcagatgaagcaatagttgctgaatc |
| | | tttgccgctcttaagtgataagctttatatgacctataaagataatccggcagaagttgcagataaggttcgtgcgcttttaaacaagtgacagctactgtcaaatgtgtataaagt |
| | | cattgatattttatataaaatcaatggattgcaatccatataagattccttatgcatcagtgacccggtgctcgcccggtcactgcttcagtcccagcagaactcagacgaggcg |
| | | cttaacatctaacgggatgccaacccgacgtttggttttatcggctatctagcctatatagaagca (SEQ ID NO: 370) |
| 2 | Retron- | cacgtaaatatgaaaactgttagcccacatagcccaacaaaaatatttgatagttaaccttctgttactaaagaaaacaggaaagtaaaagtgggctaaagcttatgcgccctc |
| | TOPRIM | gatgttgggctagccccaaaaacggtaaatttagcttaagtgcataattggttagctcaaaagcattatttttcatttaaataaattagttaattggtcttgtttagatgattcaactgg |
| | | gctgactactttctttgtatatactccggataaattttcccagctaacttgcctaatcatcactctgatgccagaaatgaacagaacgcaaaccatctataacttattgaggattttga |
| | | aaaaaattgattgggggcttgagttatatgatgactatgctaatttaatacggcacatgcaggtagatttgttggttgtggtatcgcaatcagtgttaacaaggtcgggagtattcg |
| | | ccctctgactgccgtcaagtcatcttggcgtcaccgttaaatgcgtaagagtacctgcatgtgcattaacataatcaataatggaatttactgttatgtttaaacctacctatctggc |
| | | aaggctgcaggcttgttgtaacaaatttgaactggctgatttgcttcagattaaagttacatttctgactaatgttttgtatagaataaggccagaaaatcaatacaaaaaatttacta |
| | | taaagaaaaagtctggaggagagcgggagatctttgctcctgatgaaaaactgaaagatattcaacaacgactttctgaacttctatatatatgccaggaagaaatttgggcaa |
| | | aaaataatattaaacaaaatgtatcacatggttttgagaagaataaaactataattacaaatgctgagaggcatcgagataaaaatattgtatttaatattgatattgagaatttcttc |
| | | ccatcctttaattttggtcgcgtgcgaggatattttattgcaaaccaaaatttcaagttacatccaaatgttgcaaccattattgcgcagatagcctgcctggatggatcgcttccgc |
| | | aaggaagcccttgttctccagtaataactaatcttatttgtaggattttagatttcagattatcaaagctagcagtcacatatggttgtagttacagccgctatgcagatgacattac |
| | | gttttcaacaaacaaaaaaaacatccctgatgcattagtttctaatgagaaagaaaacgaaccaggtaagatattggtagaagaaattcatcgtgcaggcttcactttaaaccat |
| | | aataaaaacagagtgtctaggtgtacatcaagacagcaagttacaggtttaactgtaaataaaaaaataaatgtaagcagagagtatataaagaatacaagagcgatggcgc |
| | | attctttatactttgaaggttcgtatacacttattgagaaagatggaaaacatagaaagggcacccttagtgaattagaagggcgatttgcatttatcgatatgcttgataaatataa |
| | | taatgtggaagcaaagaaaaatgcgcgtcctgagagatatgtggttaaaggatttgggttggattttaagcagagacttaactccagagagaaagcatacagcaaattcctat |
| | | actataaaaatttctatggaaatgagcaaataacaatcttaacagaagggaaaactgacccggtttatcttaagtgtgcaattgattctttgtttttggattaccctcagttagttaga |
| | | gaggaaaaaaacacaaagaatagagtgttaaaagttaatttatttaaaaccaatgacaagaaaaaatattttctcgatttgtctggtggagctgcagactattcgaggtttttcag |
| | | acgacatggtttactttgtaaagcgtatgaaaaacagcctcctaaaaatccagtgataattttattagataatgacacagggccatctgacttcataaatcaaataataaaggatta |
| | | ttcgcatctaccaaaaaaagcggaggatgttagaaaaggggcgttttatcacttagagagtaatttatatgttctttttactccgttattaccaggggataactattcttcactagag |
| | | gatttttttgaaccaaaagttttgcaaatgaagtataatggaaaaagcttcgataaaagcaataatcatgacagttctactacatttggaaaagatagatttgctacttatatagtaa |
| | | gggaaaatagaaaaactatcgatttttcattattcaaacccatacttgattcaattattgaaatcaaaaaacattttatcaatctacacccatcaaagtgatggttatgaaaagagat |
| | | aaaaatgctgatgtcaaaagaggcttatgctcggcacagtggagtgagctgccaaactgtcgatgactgggtagccggtggggcggaagtagttatgtcccgtagcaaggt |
| | | taagatttgctcttgtgtgtggggaaccttagtcaattactttcctggcgcactgtgttagattttgtaaaattttaaaagactaaagatttaatatcacttctccatggaggttgtg |
| | | (SEQ ID NO: 371) |
| 5 | RT- | gtggcaagattataccccatcaggcataagatgctttgacttataacgcatcagtttgaaacacaatggtgatgggggtcacaggggctgacatgtacttttaagattaaaaag |
| | nitrilase | cattaacatctacttttgaagaaaacagaaaaaaacaatcacaaacctttaaaaacaaaaactatgccaattattaataaaaagtatcaagagcttcagttaacagatgagtacat |
| | (UG1) | taccgatccactgctcatggccctagcctggaagaaaagccatcactacatacgtaccacaaattggtatgctgacaactttgaactagacctgtcggctttggacctaatgca |
| | | gcactgtaaagattgggtcaagagaatgcaggacaaaaaagaatttaaattttcagagctacaacttgttcctgtaccaaaagcctgtaaatgggagtttaagactgtcgaaaa |
| | | taaggttctatggcaaccttgtgatgaaaaagaacttaccctacgcccccttgcccatatacccatagctgaacaaaccatcatgacattagtcatgatgtgcctagccaataca |
| | | atagaaaccaagcaaggaaacccagacaccagctatgacatcgtccaccagaaaggtatcgtcaattacggaaatagactttattgtcagtatattgacgataaagcagagc |
| | | acagcttcggtgcaacagtgacatatagtaaatacttcactgattatcggaaatttttaaataggccttatcattttgcgtcaaaagcgcaaggtgaaatttcgccggacgaagcc |
| | | gtttacatcatagaactagatcttgcgaagtttttcgatttagtaaacaggaagactctaattcaaaagataaaaaaccatatcagtgagtcaataaacaataaagaaaacccact |
| | | cgccaatcatttatttaaatgttttgcaaactgggactggactgcatctagcataaaaaattatgacatatgcaagtcagacgaagtaacagaaataccaaaaggcatccctcaa |
| | | ggattggttgcagcagggtttctatcaaatatttacttacttgaattagatcaattcttgcataataaaattaacacagacataactgatgacattaaatttgttgattactgtcgatatg |
| | | tcgatgacatgcgatttgtggttaaggttaaaaaatcaaaaaataataataccgcattcataaatgatgtaataaccaatcttcttaaaaatgagatagataatcttggactgataat |
| | | taatcctaaaaaaacaaaagtagaaatttttagaggcaaatccgcaggcatctcgcgtagcttggaaaacatccagaccagattaagcggcccaatatcaatggatagcgcc |
| | | aacgaacaacttgggcatcttgagtcattattaagtctgacaaaaaccgattttgaaccaccgaaaaatggtaaatcaaatagattagctgagattgaaaaagaccgtttcgatg |
| | | tcagggaggacactcttaagcgcttttctgccaataaaatcagtaagatactaaaagagttaagacatttcatctcgcaggatatagatactgatggggaggttattgccgggg |
| | | aatgggattatctgcaagaacgtttggcacggcgttttattgtctgttggagccatgacccgtcactggcactgctactcaagaaagggctggaacttttccctgatcctaagct |
| | | attagaccctatacttgaacagctttgctcactcattgaaagcgataatgaaaaacaaagtgcagtagctacttattgccttgctgaaatatttcgacattcagcaatgactattcat |
| | | aaaaaagacacctatgcattccctgcacaagccaatgtggatgggtactttgaaaaaatacaacattgcgccgcgacattcattaataagcgcagcgcctctgacaacgaaa |
| | | cttggaacctgttaattaatcaggctagttttctgttgcttgtgcgtttagataatacattagaaaaaaatggcactgatgccaggcatgatcttatcttaaaactggcatcaggcttt |
| | | agaacaattacacttcccactaaaatggatagcaagactatagcctcatgtattttgttggctagtcaattagttaaagataacaaaccatttattcgctcctgcgcttctttgtgcg |
| | | aaagaatttatgacaaagaacacgtcataaaattgaagaaaatagttagcataatatcacatcaaaacttatcattgtttaaatccttagtttatcattcacgacctttacaacagaa |
| | | gtggctaaactcagactccgtgaaaataataattaatgaatgccatatagatatacaacctttggcgacttctttaggcatgataaaaagtagtcactcattacttagaatcatatc |
| | | aagacctgataacccatttgccaatgagataatggcattaaaactgatgcaagcccttttattggacaggattgtttgcctggataataaaaaagattatcaaataagtgtagcaa |
| | | acaccaaagtgacgtttcataactactccaaccctccaacatcgaatgtcttcgatgcaggaatggatatggatgcaaaattattcaaatcatcgggatgggtcgattctattttc |
| | | acggatgatgcagacactcaaatattgtatagagttgccatgtgcatccgttcagtactactcggcaaacaagactggacagattttggtcaagcaatttcccccaaacagggt |
| | | tatcggggtattaaaactagtagagacaaacgtcaattggggatgatgacaacacctgagtccattgccggtgagaactctcaggtttctggttggcttaccacactcttatcca |
| | | agttgcttgcctggccgggaatttcagtgggtgataatggatatcaatggccagcaatttttacagtagatgctgtcagaaaactagttgatgctcggctgagtaaacttaagca |
| | | ggattactgcaaactatcaggaactccgggacttacagaaaaaatacagttcaactggtctgactcgaaaaaagccctaacagttgctatggtccagtcaaaactgcctgcaa |
| | | cgaaagattttgtcagccatggacttcttttaaactccgcaaagtatagagtgattcatcgcagacatgttgctgaagtggctgatttagttgtaaaacacacgcttgcacaaaaa |
| | | acaactcaacgaactcatggtgaaaaaatagagaacattgatttaatagtatggcctgagctcgctgtacatagtgacgatttggatgtactcatcgccttatctagaaaaacga |
| | | atgcaatcatatactcgggcctgacatttattgagcaacctggaatcaaaggaccaaataattgtgccgtttggattgtcccacctaaaagcaatagcagccagaaagaaatg |
| | | ataagacttcaaggcaagcataatatgatggaagatgagaaaggccgggttgaaccctggagaccataccaattgatgcttgaacttgttcacccccaatttactgataaaaa |
| | | aggatttgttctcacaggctccatttgttatgacgcaaccgacatcgcgctaagtgcagatctcagggataaatcaaatgcttatcttgtagcagcattaaacagggatgttaata |
| | | cattcgattccatggttgaagcactgcattatcatatgtaccagcatgttgtgctcgttaactcaggggaattcggaggatcttacgctaaagcaccttacaaggagccgtttaat |
| | | cgtttgattgctcatgttcatggcaatgatcaggtagctataagtacgtttgaaatgaacatgtttgatttccgtcgtgataatataggaaaaagtatgcaatccgggttagataaa |
| | | aaaactgctcctgcaggaatcataatgtaataaatattagatatttttatattagaggtgaggagatggcgtcacctctaatattttcgctgattgtatttagcatcaaataataaagg |
| | | tacaattaatttaagtgactatcatgaaaaaattagttccgccatatcaagtaaccccggcacaaatctatcgttccgttgccagttctacagccattgaaaccggaaaac |
| | | (SEQ ID NO: 372) |
| 7 | RT | gcgttgaatggtataactatggcacggttaccgcatgttttgagctgtaatcgaagttatgaaaattgctatataaagcggtcgctgttgtggagatacgattgcgggaagtgat |
| | (UG3) + | ggaaagagctataaaaagtacagaggatagtttaatgagggtattatgaaccgtcagccgtttacttcagcagcacttaaacgaaacttaagtgaaagtgagaaggcttattat |
| | RT | tttaaaaaaaataatgttgctgagttagaatcattaattagtgatgccgttttaattgctaatgagaattttcgctctggtgtgagtgtaaagaaactaaatattaagggacgctgcgt |
| | (UG8) | ttacactgcttcatgtttgaaggaaaaaataatacttagacattgcaatgcaaatttaaaatgccttgaatcgcttcgtcccaaacaacgaaatacaataattagtgagcttaaaatt |
| | | tatttggaagaaggtactccattcaaaatatatcgtttggatataaagtctttctttgaatcaattgatttaccgcagctttttcagctcttacataacgaaacacgactgtctagacat |
| | | acaaaaaatttgctagaatggtatcttaaatcgtgtgaaaggcttcactcttcgaaaggattacctagagggttagaaattagtcctatgttatcagaattgtacttggcacaatttg |
| | | ataatagtattcataggcatccagaagtattttattattcaagatttgtagatgatatggtaatcgtttcaagtggttgtgaatgtgaagcgtcctttatggaatttatacaagatgtatt |
| | | accaaagggattggctttaaataaaaataaattaaaaatatctccatgcataccaaagagaagtaagggtttaaataaacaggataaattgcttcatgaatttgactttctagggt |
| | | actcgttttctataatagacacacctttgagcaaagatggtgagattaatagctgttacagaaaggttgttgttaatttatctaaatctcgcctgaagaaaattaaaacaagaatag |
| | | ctaggtctttctactcttatcatattaatggtgattttaaactattgctagacaggatttcttttttgactagtaacagggatttaaatcgcaaaataaaatcgttaagttctttagaaaaa |
| | | agcaagataagtacaggtatttattacagtaatgcgaagttagatgttgactccatatccctaaaaaaattagatgactttttgctatattgtgtgcaatctaatactgggcgtttgaa |
| | | tagtgttgcaaaaaaaccttttaatttgaagcaaaaaaaagaactgctaagaaatagttttagaaaaggctttgtggatagagtatatagaaagtataactttaagcgctatactga |
| | | gattacaaaaatatggttataaagaaaaacattaaacttgataagaaagattatctcagggctttactatgtgatacactgcccggtgattgtccaattattttttcaaatgatggctt |
| | | atatataaacttaacagaatatgatagagtttgtaatgatttgttacattttactccggtttcttctttcttaaaaaaaatagttaaccctaatttagactcttctattagtgtcgcagatcg |
| | | ccaccgagaaaagaagaaacaaagctccccatttggctattgtatagtaaaagatgcctttagccaaagacatctttctttaattcacccaagatctcaaattaattattcggaatt |
| | | ttataaaacatactcatccgttatcacattaaatactttaaaaagtaatttttctattcgctacccacgtaaggtcgctaactctttctttttatatgaaaataatgctttggaaaaatata |
| | | aaggggaagatatcgaaacaacaaaggatgagttaatgaggaaatattcatcctcttattttagttatggcggtttcaacaggatatataaactatttcaaagtaagatgtttattg |
| | | agcttgagaaaagattctcggtgatgtggatgttagatgtatcacattgttttgatagcatatatacgcattcggtttcttgggcattaaaaaataaatcatatatcaaaaaacatgtt |
| | | aaacacagcaatcaatttggacaagaattagatacactgatgcaacgtagcaataataatgaaacaaatggaatacctattggttcagagtttagcagggtttttgcagaattaa |
| | | tatttcagcgaattgattgcaatattgagtcatgccttcttagtgaacatggatgggttaataataaagattatgttatattgagatatgtagatgattttattgttttttgtaatggtgagt |
| | | caagtgccgaagttattacaaaaataattaatgtgaagttaaatgaatataatctacaattaaatgtaaacaagcttaagaagtattctaggccattttgcactagcaagacaagtt |
| | | tgattgtcaaagttaatgaattaattcgcaatttagaaattaaactgtatgaaaaacgtgatagtggctttactttaaataaaataagaagtaagcatgatttaaagatatatgtaatta |
| | | atcatgtcaagtctatatgcattgaaaatcaagtgtcttattctgatgtttcatcatatataatatcatctctttccaaaagattaatatcaataattgatatattacgagttcaagaaaat |
| | | gaagatgatgtagatgtaaaaaaaaggattaaggacttaattttcacaataaccgatattatgttgttctttttcagtgttaacccaactgtttcatcatcttataaattatcaaagaca |
| | | atggttgttgttaataactatttgaatgaaatatctagtgactatagtagtatttttatgactacgttagtgaatgctgcggaaaacattaattttggtgagaatgataatgggctgttta |
| | | ttgatgatttcatttcaattgaaaaggttaatttaatcttggctgctactttttttggagataattatcttataagtgacagtttttttcatggagttatacataaaaagaaattggactactt |
| | | tactataatctcactgctattctattttagaaacagaagatcattccgaaaattgaagtgtataatagagggtgaaataaaggaaatattaagttctaatatggatttgctgcaatcat |
| | | cggaaaaggcacatttatttttggatgtcatgtcatgtccatttgtctcaatagagacaaggcgttttttatatagaaaatatctcaagagctatgagccaaagctgaacagaagtc |
| | | atctggagattgagaatgatttgcaatctctgcttcaaacatattggtttgtcaagtgggatgagttagatattgtgaaaatgattgagaaaaaagaattgaaagaaagctattaat |
| | | ttgataaatatgagtcgtggtcagtttcaaaatacttacgtcatcgtcgtcggtgtattttatatcgattatgaagacgatttcgctggaactgaaatcggcttgaatgcttaaactta |
| | | agctaaaaaaacagtttgagaccaaagcctaaattattaggctttggattttcaggttcagttgagagtaattgctgtctg (SEQ ID NO: 373) |
| 7 | RT | agatacagtctccatcatactcagaggcgcataccccttacatatctcaggtttatctggcttaggctatgacgctaacccactagagaatcggagaaaagtaaagactgtttga |
| | (UG3) + | tttgtgagcttgattgattgcaatttaagcgctcgacacagggcaggatgccaaacaccttcaacagagaggtcggtagctccagcatatgcaagctaacgttgctttggaact |
| | RT | tcaactaagtaccaagagtggacggttccttagtatcaggcaagtatatgattgcacctagcggtgtaaagagttataaaaaagcataaaacgttgtattgtgagactttaatga |
| | (UG8) | accggcagccatttacttcatcagcacttaaacgtaatttaagcgaaagtgagaaagcctattattttagcaaaggaaatagcgaaaaattagaatcattaattaacgatgcagt |
| | | attaattgccaatgaaaattttcgttctggagtcagtgtcaaaaaattaaacatcaaggggcgttgtgtttattccgcatcgaatttaaaagaaaaattaatactgaggcattgcaat |
| | | tccaatctgaagtgtctggaatcacttttgcctaaacaaagaaataaaataattgatgaattgaagctttatcttagagaaggcacacagtttagggtttatcggctagatataaag |
| | | tctttttttgagtccatccagttgccccagctttttaaatatatgcatgatgagtcgagactatccaggcatactaaaaacctgctagaatggtatcttaaagcttgtgagcgtattca |
| | | tgccacacaaggcttacctagagggcttgaaattagtccaatgctatctgaattatatttgtcagagtttgatcgcaatatcaatcgacatccagaagtattttattactccaggttt |
| | | gtagatgacatggtgattatttcaagtgggaatgaagaccaaaagacctttatgaaacaggtagtggatttccttcctaacggtttgaaactaaataaaaacaagctaaacatat |
| | | cccctttaattcctaaaagaagtaaaggggataataataatgataaattactccataaatttgatttccttggttattcttttgcagttatagatacaccattagcaaagaatacagtaa |
| | | acatcatatatagaaagataattattgacctatcaagcggtcgattgaaaaaaataaaaacaagaatatcaagagccttttatgcatttaagaataatggtgattataagctattact |
| | | agacaggatttcttttctaactagcaatagagatttaaacagaaaaattaaatcactgagttcaactgagaagaccaaaattagcaccggaatatattatagcaacgctcggctt |
| | | gacgaaaactccaagacactaaagcaactggataactttttaatttattgtgtaatgtcaaatagagggcgtttgaatagtgttgccaagcattctttaagtataaaccaaagaaa |
| | | ggaattattgcgaaataattttacgaaaggtttttctgcaagaatttataggaaatataattttcaacgttatacagagattactaaaatatggctctaaaaaagaatattaaacttgat |
| | | aaaaaggattataccagagctttgttgtgtgatacccaaccagcagactgtccgattattttctcaaatgatgggctttatgctaatttggcatattttgatgttaactataaaacatc |
| | | aacagattttactcctctttcatctttcttaaaaaaaataattaacccatcgttggacttgtctattacggttgatgaaagagagcagaaaaggaaaaaacagagcttccctttcggt |
| | | tactgtattgttaaagattcttttagcttgagacgtctttctttaattcatccgagatctcaacttaattattgtgagttttacaaaaattattcatcagttataacctacaattcatcaaaga |
| | | gtaattattcaataagatatcctaagaaagttgccaattcattctttttatatgagaagaatggagcggaaagatataaaggggaggatattgaaactactgaggatgaattaatg |
| | | aggaagtactcttcttcatatttttcgtatggtggtttcaatagaatatataaattattccaaagtaaatctttctttgaacttgaaaaaagattctctataatgtggatgctggatgtatc |
| | | acattgttttgatagtatctatactcactcagtgtcgtgggctttaaaaaataaagcttacattcgcaagcatgtaactaacagtaatcagtttggtcaagaattagatacattgatgc |
| | | agcgaagtaataataatgaaacaaatggcatcccaataggctctgaatttagtagaatatttgccgaattgatcttccaacgaatcgacaataatattgagttggatcttatggat |
| | | gagcatgggtggaaaaataaaaaagactatgtgatattaaggtatgttgatgattttattgtgttttgcaataatgaatcgaatgcagaaataatttctaaaactattaatgtgaaatt |
| | | aaatgagtttaatctccaactaaataaaaataaattcaaaaaatattcaagaccattctgcactagcaaaacaggacttattatcaaagttaatgagttaattcaaaatttggaatca |
| | | aaattatacgaaaagcatgacggcaatattgttcttaataagataagaaataagcatgatttgaaagtatatatgattaataacattaagtctatatgcttagatagtcaggcttctta |
| | | ttcagatgtatcgtcctatttgttatcctcactgtctaaaagattaatagcacttatccatcacttttcttttgagaaaaataaagatgaagaatttaaaaaaatcaaagatgtaatattt |
| | | acactatctgatttaatgttattcttttttagcgttaatccaacagtatcatcctcgtacaaattatctaaatcaatgatcattattaatgattatttgaaagggatttcaagtgattatagt |
| | | aatatttttatgacatcattggtaaatactgctgaaaatatcaattttggtgataatgacaatggattatttatagatgattttatatccattgaaaaggtcaatttaattttggcagcaac |
| | | gttttttggggataaccacctggtaagtgaatctttttttgatgggattttgcaccaaaagaaattagattactttacaatcatatccttattattctatttcaggaatagaaattcatttca |
| | | ggcacttaagagtatagttgaaagaaaaattatagaattactatgtccagatatggatttgttacagtcttcggagaaggcacatttatttttggatgtaatgtcttgtccatttgtatc |
| | | aataaaaacaagaagatttatatatataagatatctaaagtcttttgagccaaaaaatctaagaacccactctgagattgagaatgatttgcaatcaatgctccaatgctactggttt |
| | | gtcaagtgggatgagttagatcttttaaagatgatagagaaaaaagaattgaaggaaacttattgatctgataaaacattaatgtggtcagtttcgaaatacttacgcattattggt |
| | | aagataaaatcttatgttaccaataatgtgatttcgctagatttggaatcggcttaactgcttaaacttatgctaacagaattgcttaagacctaaccattctttggaatgagatggg |
| | | gcttccaggtccagttgagagtagtcactta (SEQ ID NO: 374) |
| 7 | RT | cttgagtttgcgtaagataatttcgtgaaaattaaagcaattaatataaaaaatgtaattactagtgtgtacagatatgaaaaatgatagttataaaaccatatgaaaattgaagaaa |
| | (UG3) + | gagttcaatttttgccttgtcagtaacaaataggtagcttattgaaaaaagataaaaaattaacaaaaaatcaataaattcatatagaataaaaatattaaagaaatgaaataagtg |
| | RT | tttgcttcatcagttttagggatacattaaagtggttgataaagaaaaatattatactggattaataaaagatataaaaatagtagcttatgcaagattcaataaaatacgtcgtttaa |
| | (UG8) | agagaaataattttttaggattgttatctatttcggtagtttctatcttagttattatattatcaattgtagaaaaaatttataatataaaaacaatgagtttaattccattgtttgaaccaaat |
| | | atagaaatatggttcttttgtatacttgcttcaataattattctttgtatatctattgcactctctactatgaagattgatattgaaatagaaaggttaaataaaagtgcagttgaacttaat |
| | | gaagtaaggcggaaaattgaatttaatattgagaatagtaattatcaaaatagtacattgtttgataaatatcttgaaataataaagtcagacttaataaatcatgatgaggttgatta |
| | | taaaataaataagtatttagtcagtaaagttggtagtaagtttgcttattatcgaatgtattttattgatcagaattttacatcaatattttatctttttataacatttttaagcttttcttca |
| | | attatttcaattattttgcaggtaatgttgaagtgataagacaagattttagtgtaaattccctgttgagaatcacaactaaaaatgaaattgttaaatttaacttgggtcgtaataaggaa |
| | | gagtatgctattgcattatctcaagtttctaattatctattagagggcaatgaaataatagataatttaagctgtagaatagaaagaaataaagttatatttagtactaattcaattaat |
| | | actttttatgctttaaaaaaaatttctaaagatttaagccgattgtataaaattgagcctcctaatagagatgatatttctgaacaaatttatagaatttttgaacactctacaagctata |
| | | gtattgtaaggttagacattaaaagtttttatgaaaatattcaatataatgaggtaattaaaaagctggatagagataaaatactagttgcaaaatctattaaaattcttaaggatttat |
| | | ataactttattgataatggtttaccacgaggtttatctataagtcctattttgtcagaaatatttatgaaagaagtcgatcaacaaattagaaatatagatcatgtatactattatgctag |
| | | atatgttgatgacataatagtaatttcaacagataagagtgattctatatatgaaaaaacaattaaagttttagagaaatatgatttaaatgttaatagtaagagatatataaaaaata |
| | | ttcctgctgtgaacaataatgaaatctcaactttatataagtttgattacttaggatataagtatattatagatacaatttcatataaaaataaacgaatagttaaagcggaactgtca |
| | | gatgataaaaaaagaaaaattaaaactagaataatacatagtcttttagatagagtttataatacaacgcattatgatcgggaggagttgttaattaagcgattaaaagtgttatcc |
| | | tctaactactcaataacatataatgaattgtcaaaaactaatttaaaagctggtatgttttatagtcataggttagtaaataattatggtatttttagtgaatttaataaatttttatctaaa |
| | | gctatctactgtcaacaaaacaatttctttggtaaagctatgtcgcagattcctagtaaagaaaaagaaaatattattaaaagtatttgttttgttagtggatttaaagataaaaacttt |
| | | attgagttagagagggttgaaatggaacgagtaaaaaagtgttggaaaaataaacgatataagaagctttgaggtaaaaatgaaaagtaagatttatttagataaaaaggatttt |
| | | tatagagtattgttaactgatgtattaccctatgaagtaccttttattttaagtaatgaaggtttttatagaaacttaaaaagcaactcatttcattcagttactaaaaaaatattagaatt |
| | | aactttatttacttcacaagtaaacactaatccttttaattttaaaatctctaaagatgatagtaattttaggaagttatatttagttcacccaagttcacaaataaaaatatcaaatttata |
| | | taaaaattattatcaattaattacgcatttgtgtagtagaagttctttttcacttagatatccaacttatgttgcaaaagctttttatagtatagaaagagatagatctaattccgaaaatt |
| | | ataaagatgaagatattgaattactgtcacaaaaaagccctaaatatgcaagtacttattttgtatataaagatatcagttttttatataaattctatgattcttatagatttcaccgtatt |
| | | gaaaaaaagtttaataaactattaaagtttgatattgctaaatgttttgactcaatatcaacatttcaattacctagatcagttaataaaaattgtagctttgaaagtcatacagatatac |
| | | atagttttgaacatttattttcttcaattatgaaaggtgcttatcatggtaatacacatggtattgtaataggaccagagttttctagaattttcgctgaaattttattgcaatctatagatg |
| | | tagcaataaaaaataagttaagaaatgaaatgggaattaaggagggtgttgattatgttataaaaagatatgtagatgattattttttattttataataatgagcaaacttcaaatttaa |
| | | tttttgaatgtattgttgaagaactttctaagtatagactattttgcaatgaatcaaaaagtattaggactactattccttttattacaggtattactattgctaaacatgaaataaggaa |
| | | gagattagaaactttttttgaattatttgagtcaataaataataaagatgattatattgggctaaaattaaatcattattataaaatatcaaatcaattaattagtgatattaagtgtattgt |
| | | ttttaataataatgtaagttattcaagtatttctggttatttttttactttaatgaaaaatcatgttttgcatataaaaaatagtttttcttttgaggataaatctaaagttgaaaatttaagt |
| | | aagttatttcttattattcttgatgtttcgttttttgtttactgtatgaattttaaagttagaagcacatatttaatttctcaaattatagttttgattagtactattgctgaatcatttgatt |
| | | taaatttgatagatttaattaataaaaaaatatatgatgaggtggatttggttttaaagataaagtcaaattcaaacttattgaataatattgaaattttaaatctattaattgctgttagaga |
| | | tattgatcttaattatcagatcttagtagatgatcttatgttattgttttcttcagaaaggattaataagtataattatttctctttaatgacttttttattttatgttcaaaggaaaaaacag |
| | | tatcagcctatcagagatagaatttatgcaataataattcaaaaatttaatcagaataatctaaatgtctcaaatgattctgagttaattcacattttttttgactcacttagctgtccttatt |
| | | taactaaaaatcaaaaaattaatataactaactctgcattaaattctattattaaattaaatgataatgaaattgatgtttttgtagaagaaatgagcaaaactaattggtttattgactggaa |
| | | cttgcaaacaaaagatgcaattcagcgtttgctgatgaaaaaagaattgaaatcaccctatgaaaattgagataattaagctagaaactagatatacctccgacatttgttggttgatt |
| | | ttacacactatataactcctagtttctataaaaggatgtttctaacatccttttattttttttgagatttaatttttcttttagtgacaactaagttttactataactaatagc (SEQ ID |
| | | NO: 375) |
| 8 | RT | aattccccgaaaatccgcccgtttttactgaaaaaagccatgcatcgataaggtgcatggctttgcatgcgttttcctgcctcattttctgcagaccgcgccattcccggcgcgg |
| | (UG15) | cctgagcgtgtcagtgcaactgcattaaaactgccccgcaaagcgggcgggcgaggcggggaaagcactgcgcgcaagctatgtgaggtgatgtgtaatacatatcacg |
| | | aatagcgtaggtagctgttggctttgcctgatcaaggtgacagtatacatatcttaaaatataaatatttatgattatttatttgaaagaggttgaataatgatttttgatgaaaaaaga |
| | | catttatatgaagctctgctgcggcataattattttccgaatcagaaggggacgatttcagaaatcccaccatgtttttcttcaagaacttttacaccagaaatttgtgaattaatagt |
| | | ttctaatgagccggggaaaagaaaattacatggatacgattgtgtcgaatactcatcgactaggtataataactttcccagagtattatccttaattcacccaagagcatatgcac |
| | | agttagcaaagcatttgtatgagtcttgggatgagattcgaaaaatcaaagaaaataaaaacagtatgattaaacctgaaatgcatcctgacggtagactttttatcatgaattat |
| | | gaggatgcagaaacaagaactgtaagggagttaaacgatggatttggaagacgatttaaagttaaaactgatatcgcaggatgttttaacaatatatattcacactcaattcctt |
| | | gggctgttgtcggtgtgaataaggcaaagacatcaatgaataagcataaaaatagccaagatgttcattggagtgatagattggattattatcaaagacaaacaagacgaggc |
| | | gaaactcatggtgtccctgttggacctgcaacgtcaagtattgtatgtgagataatattaagttccatagataatattcttgagaataaaggattcttattcagacgttacattgatga |
| | | ttatacatgttattgtaaaactcatgatgaagcgaaagagtttctccatgttttaggtactgaactttctaagttaaagttatctctaaatttgcataaaactaaaattaccagtcttccc |
| | | agtacattgaatgatgattgggtgtcgttgcttagtattaactctccatccaggagagtattcaggaataatgactcggatatattatctgcatctgaggttataagctttttggattat |
| | | gcggtacaacttcatctgacgaatgggggcggtagtatattaaagtatgctatatctttaattattaataaagtagatgaggcgtcagcaagagagatgtacgactacgttttaaa |
| | | tctgagttggcactatcctatattaattccatatttagatgtattgcatccaaagattaacattaatgatgaggtcaggttaaaacttaatgaggttttgaattcctgcatagataataa |
| | | gttttctgatggcatggcttgggtgttgtattattgcttaaaatattccattgatattgacagttgtctcattagtaagatttttgaaaacggtgattgcctaagtatttgtattttggataa |
| | | aactggaagatatgataaggaaatagaagaattttctaaaaatataatttcattggattatttgtatgaggttgataaatattggatattgttttatcagcgattctattcagggaaagg |
| | | atataatccttacaatgatgattgttgtttcgatataatgaaaacatatggagttaattttatgcctgatgatggttatcaaacgaaagctgaacactattgtaatatagtaaatagtcc |
| | | atttcttgagaatgatgaacaagtaataagttttaacgattattgttcataatttataattagcctccg (SEQ ID NO: 376) |
| 10 | ATPase + | actgctcgacaaaacgaaccgttcattcgcgaggatggtggcagtgaatgaggtggtcagttttatcagcgcttcaaggtagctttataggatggattgtagcgaagtgccca |
| | adenosine | acaaattgattgaagctaagggcattgagcattgcatgcatcatgctcagactgacaaaaaatcaaaataaatggattgatacggacatgacagacagcgtacagactgaaa |
| | deamnase | ctaccgagggaaaaatcatcatcaacttgtttgctcccaatcttcccggaagtaccaaagaagatgatctcattcagaaatctctgcgtgaccagttggttgagagtatccgaaa |
| | (RADAR) | ctcgattgcttatcctgacaccgataagtttgctgggctaacacggtttattgatgagtccggccgtaatgtattttttgtggatggtactcgcggtgcgggtaaaactacttttatc |
| | | aatagcgtggtcaaatctctgaacagtgatcaagatgatgtcaaagtcaacatcaagtgtttgccgaccatcgaccccaccaagttgccgcgtcatgagccaattttggtcact |
| | | gtgactgcccgtctgaataaaatggtgtccgacaaattaaaaggatactgggcgtcgaatgactatagaaaacaaaaagaacaatggcagaatcatcttgcacaacttcagc |
| | | gtggtttacatctgctgacagacaaggaatataagccggaatatttcagtgacgctttgaaactggatgcccagcttgattactccattggtggtcaggatttgtcagaaatcttt |
| | | gaggagctggttaaacgcgcgtgtgaaattctcgactgcaaagccattttgattacttttgatgatattgatactcagtttgacgcgggttgggatgtacttgaatctattcgtaaat |
| | | tctttaacagccggaaattggtggtggtagcgacaggtgacttgcgtctatattcccaattgattcgcggtaaacaatacgaaaattacagcaaaactttgctcgaacaggaaa |
| | | aagagagcgtccgcttagcagagcgaggctatatggttgaacaccttgaacagcaatatttattaaaactttttccggtacaaaaacgtattcaattgaaaacaatgttgcaattg |
| | | gtcggcgaaaagggaaaagccggtaaagaggagatcaaggttaaaaccgagccaggcatgcaggatattgacgccatagatgttcggcaagcaattggcgatgctgtta |
| | | gggaaggccttaatttgagagagggatcagatgctgacatgtatgtaaatgaactgctgaagcagccagtgcggttgttgatgcaggtgcttcaggatttctatacaaaaaaat |
| | | atcatgccacatcggtaaagcttgatggtaaacaaagcagaaatgaaaggcctaatgagttatcagttccgaatttacttagaaatgccttatatggctcgatgctaagcagcat |
| | | ttatcgtgcagggttaaattatgaacagcatcgatttggtatggattcgctctgtaaggacatttttacctatgtaaagcaggatcgtgattttaacactgggttttatttacggcctc |
| | | agtcagaaagcgaagcattaagaaattgctctatttacttagcgtctcaggtgagtgaaaactgtcagggcagtctgtcaaagttcctacagatgcttttggttggttgtggctct |
| | | gtcagcatattcaaccaatttgtgaccgagttagcacgagctgaaaatgatagagaaaaattcgaacagcttattagtgagtatgtagcttatatgtctgttggcagaattgaaag |
| | | tgcctcacattgggctaatcgatgttgtgcggtggttgcaaacagccctaatgatgagaaaattggtgtttttcttggcatggtgcaattaaatcgtaaatcacgacaacacatgc |
| | | ctgggggttacaaaaaatttaacattgatactgagaatggcctagcaaaagccgcaatggcgtcttccttgagtacggtagcttcaaataatcttatggatttctgtagtgtttttaa |
| | | tctgattggtgctattgcagatatctcagcatgccgttgtgaaaggtcagccattactaatgcttttaataaagttatagctcagacaacatgtattgttcccccatggagcgaggc |
| | | tgctgttcgtgcagaaatgaaaggctcaagtaaaagtgcagataacgatgctgctgttttggatgtagaccttgatcccaaggatgatggcgtgattgatgaaagtcagcagg |
| | | atgacgcaacggaattttctgatgccattactaaagttgagcaatggcttaaaaacgtaaacgaaatcgagattggaattcgtccgtcggcacttttgattggtaaagtatggag |
| | | tcggttctatttcaaccttaataatgtagctgatcaacataaaaccagactctatagaaatgcagagcatggacgaatggctagtcaatcaaatgccgcgaaaattatgcgtttta |
| | | atgttttagcatttcttcatgcggtattggttgaagagagtttatatcattcggttagtgatagggaatatatcggtgaggggttaagactaaatccagttacttcagttgatgagttt |
| | | gagaaaaagataaaaataattggtgagaaattaaaagcggataataaaacatggaaaaatacccatccattgtttttcttattaattagctgtccaattctacatccgttcatttttcc |
| | | tgttggtgggattaattgttcagtcaaagcactgaacaaagaaacaagtttcaataagctgattgatgaaattgttggcgataaattactttctgatgaagaatgggactatctgac |
| | | taaaaataatgatcaaaaaacaaacactagacaacaaatttttcaaaatactataacatcgctgaattcctccacaatcgtcggagcatcatacgataaggatacaccagccag |
| | | gaaaaccaagtcacctttattaggtgatagcgaagaaaaatgataatggccttcgtataaggattgggtatggaaaggtttcttcttaactcaacagttctgttatataggctaag |
| | | cacagtctctttggatgaggtatcacttgatgagagagtggagtcatctgtattccttgctcaatacgaacaggctcgtagtttacctgatcatgtagctaaatctgcttggtcatat |
| | | ttagtgcaacaaatcaaacagcggaatatgaaactcggcccagtagcaatcttacgcctgatagctgaaaagtttattaaaaacgagaaaggtggccccaaaatcgatctac |
| | | ctatgttctcggaatggcaaacgctgatgagtcgagtatcgtgtctaccaattatagcgtgtcatcaggtatttaatccagggccagccagtcaggaatatagttttcgctggcct |
| | | ttatacccatatcacccgacggttgaagactacattacccgtgaatgcttacatgaaactcaccaacacctaaatggcagtaccagtgcagaagagtgttggctggatgcact |
| | | caaacacccagaagcatgcctcagagattttgagaagggctgggcatctcaagagatgaaacaactctgcgcccagattgatccatctctgacacctagaatcttcaaggat |
| | | cgtttgcaaatcgcctgtaatattcgcgaaattctttgtcgggttgctcagggcgtggaattgccagagtggatagcatcaatgcaaaatccgcagcaactggcgaatagcac |
| | | aattctgcataatggccgggagtatgggtttgcgacagtttggccaattgacgacaaatacagtcaggagtctgagttttgctggctaaccggattgttggaaaaatggcggttt |
| | | aatgcgccagaagggttagaacgattgctttggatttacctgctgattcaaaatcagtacttgaccttactggttcagcgagacgattttttcggatttgaacagttccagaattac |
| | | accatgacggagttgagggaggaaacagagaaatcttatttgtctcgttttaaacatgctcatggtgcaggagtgtattctcaggtgcgttatctggaaggacgttttgctccga |
| | | agagcgaccccaacaaaatgcaaaagctgctcttcagtgtgttaagaggatattgggaatatctgagtgctcatatgtccatggaatgggtgcatgaaaagcctctgactatat |
| | | cgcaagtgctcgataacctcgaactggttgaacctcatggcaagtgtgtagagctggcgctagtgccgcactttatcaaaagaaagcccaaaaatggtgaggcctatcctca |
| | | cgcattactattcaaagacctgaaaaatcaggcagctattctgatggacatgctgaagtctgaaccgcgtctgacaggctggattcgaggagtagatgccgcagctaatgag |
| | | atgcacgcaccacctgagttattttgccccttgttccgggtactagccaaatcaggtattgctcattttacctatcatgttggcgaggactttccgcatctgatcagtggtattcgct |
| | | ccattgatgatgccttgagatttttaccattgcgtaatggcgatcgtcttggtcactgcacggcgattggtattacacctagcatctggaaacgctctttgccattgtccttatccat |
| | | gaccaaagagacgagattgctcgatttggtgtttatctggcgggaacttcgaagtcatccggaactgctgcgttacgctagtgatgcagcgattgaagctgttcgcttggctca |
| | | taaagtgttttcgctggaagaggaagtctcgattaccacccttgatcaggtatttgaaatgcgggggctgttggccgaatcggaaggcctactgagtgagctaaatgaaccatt |
| | | aaaacccaaatccctctggttggaagagtatgagcgcgccagagagttggttaaaacaacgggtatgaaaaggccgttgaagttgtataagcaatggctaacatctgacaat |
| | | gtgcgaaagcagcgtgctgaatatgttgaagttgccctagaatatttgccggatgaagcagttgttgcattacaacaagctgtaatggcaaaaatggcagaccgaaacattgc |
| | | gatagaatgcccaccgaccagcaatacacgtatcagtcagtaccgaaacgtcagcgagcatcatatctttcgctggatgggcttgccgggtgaggcgattgaaggtgatgtt |
| | | cctatgtctatttgccttggctctgatgatccggggatcttcgctgcggacttgaaatccgagttctatcatctgttcgttgtgttaacccgaaagttcggtttgtcgccagcagatg |
| | | ctttgagaaaggtagctgaggtgaacgagaatgggcgcatttatcgctttcatgatgtcagctagcctgtatacattgaggattctgtaattgttcaagaccagcagtgctcattg |
| | | ctaactatctat (SEQ ID NO: 377) |
| 13 | STAND | aaatctctttcgcgtcaatagtggtaatatttttttatcattgtcctctttctactgacatactgattgtccgacagtggagccagtcgaaattgttgacagctagtcggggctcgtct |
| | | ggtctttctagcagtaagaaacgtattaatattggatcgccactagtttaacagatacctcagaattatttatagactgacaccaccccggcagacgatcctgccctataggaag |
| | | ctaagtggaaacttatccagtaacagcttgtcgattttatcccagagggtgttcctcaggatgtatcgctgaaatcaaatccagcactaagaatgaggggtgagaaaccatttcc |
| | | ttggtgggtctttgaccatttctgttgaactaatgtttttgggttatcaaggatacaaattcaaggcagtgtttcactaaaccttacctcgcttcaataccaatacatttttaatgggtat |
| | | aatatgtgactgcttttgccgcattattgacaggaacaaggactggtgatgaatattgatttcagtttaattcgtagcgcccccaaaagccgtaacgatagctttgaagcactcgc |
| | | cgtacagttatttaggaaaacctgtcgagtaccgacaaattcaacatttattagtctgcgtggagatggtggagacggtggcgttgaggcatatttccgctcaccggacggtgc |
| | | cgtattcggtgttcaggcaaaatactttttccagcttgcttccgcagagcttacacagattgatagttcccttaaagctgcgctaagcaaccatcccacactaaccgaatactgga |
| | | tttatataccgtttgacctgaccgggcgtgttgctgcgggaaagcgaggaaaaagccaggcggaacgctttgaagaatggaaaagtaaagtcgaatcggaagcgtcagcg |
| | | aaagggaagtcactttctattgtcctttgtaccgctgctgttatctgcaatcaattacttgagatagacccttacggagggatgcgcaggtattggtttgatgacacgttgctgaca |
| | | acagctcaaattcaacaatgtctggaggacgccattgcttttgccgggccaagatatacttcaatgctggatgtggtgacgaatgctcatgtcggcctggatttctttggtggga |
| | | ctggtgacttttgcgagtggtacgaaacatcattaacaccaatcgttcgagagttccattcactgaatggatacggacgcaaatcgctggatatactcggcgaaacccgtgcta |
| | | catctgccacggcattgattgaagaaataattgcctactgtgagagcatgagagataacaatgtcacggccacatcggttacagatctttccgtcgctctgtcatccctattgac |
| | | acttttcgctgatgcccgccatgctcaagaagataaattttatgaaaagcatggcaagcatagtgatacagaatcgttccgacagttccacgcagagtatatgtgtgcatttcct |
| | | gccggagatatggatgcggcgagaaaatgggaagagcaggcgcagcaactgcaaaatttgctgacttctcaggtcattggtgccgcaacagcacattccttactgctggtt |
| | | gggccagcgggtatcggcaaaacccacgcgattgtcagcgcagcattgcgtcgactggaacatggtggtttttcactggtcgtctttggagacgactttggcaaagcagag |
| | | ccttgggaagtgctacgcagtaaaatagggctgggtgccgccatcgatcgttcgacattatttgaatgcatacaggcctgcgccgaacatactggcttaccttttgtcatttatat |
| | | cgatgcattgaacgaaagcccgcgagaagtgcgctggaaggacaagcttcccgaattgctcgctcaatgcaagtcttatccagacatcaaaatctgcgtttcaacccgagat |
| | | acctatcgcaatcttgtggtcgattcacgctttccagggtttgctttcgaacacatcggtttttcaggacatcaattcgaagcggtacaagctttcgcagcctactatgagctggat |
| | | gcagagattacaccacttttttcacccgaactcggtaatcctttatttttacacttggcctgtaaaacgctaaagggcgaaggccgtgacagtctggatatttctttgccgggtttta |
| | | cctctctgtttcaaggacatctcaaacattgcgatgttttaattcgagaacgcctccactacgcaaaccctcgtaatctggtaagggctgcaatgatggcactcgcgaaaaccct |
| | | gacacatgagttgccgcagaaccgaacgtgggaaacctgttgcgaagcactgagcaaaatagtgggaactgagaccacacctgaatcctttttaaatgcattggcacatga |
| | | aggcctcattatcctttctgttgtagatgaggataccttcctgatccgtctgggttatcaacgctacggtgacatactccgtgctatcagccttgtggaaactcttgattcggataca |
| | | gtaaaactagcggagaaaattgcagcgttaacagaagaagatgctggattgctggaagctcttgccgccgtgctgccagagaaaactgctcttgaaattactgctgaagaag |
| | | taggattaccatccgaacaagcccataagctgttcatccagtcattggtttggcgctcccgacaaagtgtagtggaagaaattgatgaacacatccatgcagcactgcataca |
| | | cctggattatgggagtcggtttatgaagcgctgttttcacttagtctggttcctgaccatcgtctaaacgcaactaactggctggggccatttttacggcagtcatccttagctgaa |
| | | cgtgacacctacttgtcattagctgcgctgggatcatttgataataagactgctgtctattcactcatccatgcagcactatttgctgacataacccattggcctgctgaaagccg |
| | | gaggctggccagtctaacacttgcctggctcacttcgtgtgctgaccgccgaatcagggatttatcctcaaaagggctaagcagaatcctggcaaactacccggagaactgc |
| | | caaacagtaatcagtgaatttgcatattgtgatgatgattacgtattagagcgtattagccttgctatctacagtgcatgcttattgtcataccaacgcagaaatgcgtttatgccag |
| | | cgctccctggtctattaagcattgcgtcagatagcaagaatattctgctccgggatacggttcagctattagtaaacttgttgaaaacaggagaatttcccacagccgtaacaag |
| | | ccaattacagcattaccagacaaacgtatcattaccatcacgatggcctgtactggcggatgtcaaacccctcctagatctggaacatttaccatcaaacatggtgctctgggg |
| | | agaatccatggccccggatttctggcgttatcaggtggaatcgaagatttccggctttgacttggagagcgccaatatcagccatgaaaacattgcctgttggttaatgcgaga |
| | | agcacttaatttaggatatcccggttataaccactgcgcgctcaattatgatcgccatatcgggagtcagtatggctcgggacggggtagaaaagggtatgctgaccgactcg |
| | | gtaaaaaatattactggatcgccttacatcgactactgggcattctggccagtaatgttcccgcactggaagacccatattccgactacgaacctacaagtgatcttctatggtc |
| | | agtcgacgtccgtaaagttgacctgaccgatgtacgcgatatcaccgcagaaggtgtctatccagtactgatggaggaaacaaattatgcattccctgaccacaattcagatat |
| | | caaaggttgggttaggaccgatgattttccaccttatgaagcttgtcttattcgaactgacgaggaaggagagcagtgggtagcgctttcacatagctattgggatgacgataa |
| | | agcgccgaatgaaaatagctgggattccccgtacttgggagtgcgtgcttcctactcaagcgcactcataaatgaaagcatccagaactttaaacagaaaagatcacgcgat |
| | | attttccaatataatcagggaagtagttgttatcgcggttatcttgctgaatatcctgacagcccggtatacaaacaacttcttaatagtgatgaagatagtgaagcgtttaattttac |
| | | agaagtcagtttactgcgcggaaacgaatgggaatacgactactcatataccatgcccgagcgccaggataacctcattgcgccatgcctgggaattattcaaaaactcgaa |
| | | cttttatgggattgtcaaagcggttgggttgatcattctggcaaacttatcgccttccatcaaaaaggtgtaaaacaacgcggacttttcatccatcgttcggcattgaacgcctat |
| | | ctgtccataacaggtgaagagcttatacatcgccgttttgctaacagaggatattttgatttagctggtcgtaatagcacgcaaatagacctgaaaacttggatccagtaccggg |
| | | cagacaaggcaccggtagttttacgagaagaggaactgccgtttaactgctgacaacgatacttattaagtaatcaactggctgccttggcatcgaatgccagaagagccatt |
| | | tcgcactaccaatttaagtagactgaaggaatacttggtacaagcaaacgcacgccatatcggatagaggggact (SEQ ID NO: 378) |
| 21 | Trans- | attatctgccaaccgataagatggctgcctaagtcgtagcgattcagcactgttttagcggcgctcgattgcaaagtcgtgctttgctgacttgcgattgtgctctttacgagcaa |
| | membrane | agctttcaggtatagtaagtgctaactgtagtgtaaaattatagggatagatgaagaaaacaacgaggctttagctaatctttgcagttgtgtctgctataataaggcgaaatttta |
| | ATPase | tctgcatgattttgtttgattaactccgaaagccagctctctcggtgaagattgggaagggatatcaatgagtgatgatagctataaatttcaaaagttaacgccgttcagcgatgt |
| | | tgagctgggtgtatataaaaatgcgatagattttgtttttgccaataacgatctaaaaaatgttgcgatatcagggcaatatagcgcaggaaaaagtagtcttatcgaatcctataa |
| | | gaaaagtcattcaaatataaagtttgttcatatctcacttgctcatttcagatcgattgaggaagctgaaactaatgaaccaagtaaagatataaatgaaaccgcgttagaaggta |
| | | aagttcttaaccagttaattcaccaaattaatgctgatgatattccccagacacattttaaagtaaagaaaaaaataaaaactaacaacattgtgataaacaccatctttacggtgtt |
| | | atttatcgccatgatactacatatcacgctatttaataagtgggaaaagtttgtttcacttttatctgaaggtaatataaagacactacttacattatcaactaaatacgatacgctttta |
| | | attagtgggtttatatgtactatcctatcttgtattttcatttacaagttaataaaaacccaaaagaatcgtaatgttcttaagaaaataaatttacagggtaatgaaatagagatttttg |
| | | aagaaagtaacgagtcttatttcgatagatatttaaatgaagtattgtaccttttcgagaacgttgatgctgatgccattgtttttgaagacatggaccgttttaatagtaataacatct |
| | | ttgaacgtcttcatgaggttaacagactggttaatattcaacgggacacagcagggcacaagaaatcgacgttacgttttatttacttgcttcgtgatgatatcttcatttcgaagg |
| | | atagaaccaaattctttgattatatcattccagttattcctgttgttgatagttctaactcttacgatcagtttatcacacattttgatggtggtggtattctcaagttgttcaatgaaagat |
| | | ttctacaagggatgtctttatatattgatgatatgagaatattgaagaatatttataacgaatttcaaatttattataacaaattaaacacgacagaacttgactgtaataaaatgttgg |
| | | ccattattgcctataagaatattttcccaagagattttagtgagttgcaacttaatcaaggtatggtttataccatatttagtgaaaaagacaaccttattattgaagaaataaagaaa |
| | | atagaaaaagatattagagatagaaaaaaagagattgaggcaatcaatgatgaaatactcaactctagtcaggaggttgatgctatatacgataaggaattatctagatataata |
| | | atcatcctcactataatcaggctgagaaagctgatatagcaaagagaagggcggctagaaaagaaagtgttgaaaataaatttaatggtaaaatagaagaaattaatgagctt |
| | | atatcaagatcaagagaaagtttggttgattctagaaacaaaagacttaaagaagtaataactagagaaaacattgatgaaatatttaaactcacctataccaatgaaattggag |
| | | aggaaagagactttaatgaaataaaaagcagtgagcattttgacttgcttaaataccttattcgtgatggttatattgatgaaacctataccgactatatgacctatttttatgaaaat |
| | | agcctgagtcgaattgataagatgtttttacgcagcattaccgatcaaaaaggcaaagagttcacttatcaactcaagaaccccaagctggtcgttgcccgccttcgagaagtg |
| | | gattttgaacaggaagaggcgcttaattttgatttattagcttatctgcttcaaacgccagcccaggtaaacttaataaaacgtttattcaaacaactaagaaaagatagaagagtt |
| | | gagtttattcgtggttactttgaaactgagagggctcagcctgtcttcattaatcgattaaatacacagtggcctgagtttttttcttatgcgctgacagagagtgaattttctgctgat |
| | | tgggttaaactctactctataggcacgttttattattctgccaatgacgccatcgaggccattaatattgatgattgtctgactgattacatctctgattcggcaggttatttagcaata |
| | | tcagaaccgaaggttgacaaattaattagtggttttaagttgcttaacgtctcttttgtcagtattaaatttgaaaacgcaaataaagtactctttgatgcggtttaccagcattcactt |
| | | tatgatattaatttttccaacctgaccttaatgctgagtaaggtttacacgcttaatagtgaagatgatattcgccataagaactatacactagtgatgtcacaacctgattctccctt |
| | | ggctagttatgttaataaccatattagggactatctggatatggttttatctagttgtgatggttcaatcgtggatgatgaatccattgttttatccgttcttaataatgagggaatatct |
| | | gatgaacaaaaaggccagtatataaacgctttgcaaactttcgtgacatctctgagtgaggttgagagcgaatctttatggtcatctttgttggataaagatagagcagtgtgctc |
| | | tgaggaaaatattgtctcttattttgaacatgttgatggactggatgactcacttatcgaatttatcaatagaactgatgtagacctgaattttcaaaatattaatattgataacgagct |
| | | taaaggtaaattatttaaatcgattgttatctgtaatgatttatcaaatgataaatatgaaaaattaatttgctcactaaatattatttgtaaaacatcctttagcgctagtaatatcgcga |
| | | gtgataagttcaaaatattagtggataaaaatattattcgtatgaatgttgcgccacttaatttcatacgagataactattcagagcaactttcctattatattcataagaatatcaggg |
| | | catacgttgaattaatgacgattgataactttattttggatgaggctatatcaatactttcttggaaagttgatgatgatttgaaagttaagctactcgagtttgttaaaactccgttgg |
| | | ctatttatagtaagaattactctcaggtcgttaatgactatattttagaaaataattttaaaccagatgaacttctaatcttgacgtcatcttataaaacttggggaacctctactcagtc |
| | | gctcatcttgagtcgagcaatacaggatatatcagcattgatagcaagtcctaatgatgtttctgaaccgttactaaaaaacctgtttgtcgcagagggactgaatatgcagaat |
| | | aaaatagcactgctaatcgctttgttgccgggtaaggatttgagtaagacgacttgcaaagagtatcttgatctgcttggtttatcggagttcagtaaaattttggggcgaggcaa |
| | | acctaaaattgaagttgattcaactaatcaaagtttattaacagcattaagagataaccacttcttctctgattttgaggtggataatgaaaatcccacttattataaaataacaagg |
| | | cggcgctctatgtttggctcagatacatagcattatgtatttttctacagtttgggcacttttatagtgcccaatttttacgctgaaacttacgcagataatctgactttttcccagttga |
| | | cgagtacacctag (SEQ ID NO: 379) |
| 22 | ATPase + | atctatagcagtcatcatattggattattggtgaagtggtacactgaatttgcccacctgaacagagttggttttatcaaacctgtagtttactcaatgacgtaaaaattggtgatgt |
| | QueC + | aaaggatataaaaatgtggtcagacaaagagtcatcagaagactacctaaattttggtgaagtatctcagttagccgtggatgtacttaccacgaaagatatgttaccagtatct |
| | TatD + | atcggaatttttggaaactggggggcaggtaaatcctctctgttaaaactgatagagcaaaaacttgagcaagacgacaaagattggattgttatcaattttgactcttggctcta |
| | DNAse | tcaggggtacgacgacgcccgtgccgcacttcttgaagtcatcgctacagaattgacaaaagctgctgaaggtaattctacccttatatcaaaaactaagagactccttagtcg |
| | | agttgatggttttagagctatgggattactagctgagggtacagctttaatggcaggattacctactggcggtttgctttctagggggattggtgcattaagaaatatcaccgatg |
| | | gcatccagagccaggaagagtatgaggctttaggcaatatagctaaagaaggtaaagaaactgcttgtggtttgattaaaccacaaacaaaaaaaagcccccctcagcaga |
| | | ttgatgcctttcgtaaggaatatggggaaattctagaagaacttggaaagccactcattgtggtaatagataacctagaccgctgtctccctgccaatgctatccatacacttgaa |
| | | gctatcaggctattccttttcttgactaatacagcctttattattgcagcagatgaggacatgattcgctcttctgtggctgattacttcaaaggggcatcacagcgccatcaaata |
| | | gattatctggataagctaatccaggttcctattcgggtgcctaaggctggggtccgtgagatccgttcgtatctgttcatgctttatgccattgaacatggcttagaaggcgaaaa |
| | | aataactatgctccgtgagggcttagagaaggcgttacagcaatcctggaaagatgaaccaatctcacgtcaggaggccttaaaaatgactggtgaagcggatgatagcaa |
| | | cctcgcgctggcgtttgcgcgtgctgaccgtattgctcccattttagccaactctccaattattcatggtaatcccaggatcgttaaacgcttgttgaatgttgtgaaaatgcgatct |
| | | caaattgcgaagcgacgagcaatgcctttggatgaagcaattattactaagctagtaatttttgaacgctgtgttggagtggatggcaccgctgatttatatcatctcgtggatatt |
| | | gaacaaggtgttccccagatacttaaacagcttgacgataatggcggtcaaatacctactgatgcaccaaagacatggactgatagtccaacgactaaatctttcatcagtcaa |
| | | tgggcccaacttgaacctcgtcttggtgggattgacttaagggccgccatatatctgtcccgagaaactatgccaataggtgcatatgtggttggtttatcgccatctggacgg |
| | | gaagtactaaatgcactaattgaattgaaaaacactagttctcctacagcagaaaaccttttgaaagcacttcctcgtgaggagcaaatacctgtaatggaaggtttaattaacc |
| | | agttacggcaggtatcagattgggatcgtaagcccagaggcttttccggcgcatgtctgttggcccgctactcaacagatgcagccagcatattaattcgttatctacaggaatt |
| | | acagttggggatgaaacgaccagcgtggatgactgcagcattaaaagatgaacaatggaataaggacgcttaatgggaacatcacaatcaagtaaaggtccaggaggtgg |
| | | ctctccgctggttccaccatgggctgatgatcagccacagcaaccgttaccctcgccgcaagaaaggaggtttgcgccatttcgagaatcgttgggaaatgcggtatcaaat |
| | | ggaaatcgagcagatttcagaaaagccatagggcactacgcgcgaaaagcctccggagggagcagtaacgctgctcggcgattagggagtgtcacgcaagctggggcc |
| | | gaattatttggggctttagtgggaatgccttcggctcccggagaaccaagcatcgatttgggcagtttggcaggccttccatgcgaaatagcaatatcaactattgctcaagctt |
| | | taacatcacaggatggtgactcagaaaagatctgtgcggccatgaaccatgctttagtggaggctcttgatggcgtagaaattttcgatcctcaaaaaataactgatggtttgat |
| | | tgttgacacaatgattggttatctagcggaaagtattttccttcagatggtaatggattctaatagggcatggaacaaagcagatacaccttcaaaggcaattcatgcagaaattg |
| | | aactccgggaattgattaaagttgttgttgataaacatatggcaccaaaacttgccggtaacataagatcgttcacacgaaaccaaatggtaaaaattgaacgtcaggccattat |
| | | tgaggcctggcaagaatgggaggcataccagtgacacaattagttttccatcataaacatcaccatttgccgccagcaagtgagaaagtgttacctgttcagctatatggatta |
| | | agtggtcagaggcgcggagatatatctgttatcgggaatcctgcgattgatcggatcagacgtttgggagtacagcttccagctaaggtcatggattttctgagtgttgcattag |
| | | cagtaactgcagcagatactttcgttcagcgtgaaagttccgaggatggttggacccgccaattgtcgttacgactcccccttcatgaaccatccagatggattagtctaaaga |
| | | aagaacttgagagtgctttgcattttcttagtggagacatctgggatttcgaattttgtgacgatggttatgcaccgccagagccttatagccagcattcaaggcatcgtctgatta |
| | | agctaaaagggcttgactgtgtcagcttattttcaggaggtctggattcagctattggtgcaatagatcttctggctgcagggcgcgctccacttttggttagtcatgcttataaag |
| | | gggataagtctcgtcaagatcagattgctgaaaaattaagtggccaattttcgcgctttgagattaatgctgacccacacatttatcaaggcgtgactgatattacgatgcgaact |
| | | cgtagcctcaattttcttgcccttgcggccgtaggtgcttgtgccgtacaagagatatctcaacaagaaaagattgatttgttcgtacctgaaaatggatttatctcattaaatgca |
| | | ccacttactccacggcggataggttcgctgagcacacgaacaacacatccacattttattacgagcatacaaaagatctttgatgcgctcggtatttcttgtcaaataatcaatcc |
| | | atatcagtttaagacaaaaggaaaaatgatctccgaatgttcaaataagcagctcttatctaaaattgtggaaagtacagtatcctgcagtcattggaaacgaatggggcagca |
| | | atgtggggtatgtataccgtgtatcattcgacgagcatcacttcatgcagggggaattagtagagatgttgaatatattttccagtccttagctaaagtaatgaatgaaatagatc |
| | | gcagggacgacctgatcgcccttaggattgcgatcacgcagaaatcgactttgaaaataggtacatggattgccaaaagtggccctttgcctacggcagaatttgataatttca |
| | | agcaagtatttaaggatggcctagatgaggttgaaagctatttactgagtgagaacatagtatgagcatcgatatgcactgtcatctagacttatatcctcggccagacctcgtg |
| | | gctgaagaaagtaaacgtcgagggacttatattctgtcggtgacaacaacacctaaagcatggcatggtacttctttattggctaaagaaagtcaacgaatccgaactgctctt |
| | | gggctacatcctcaaatcgcgcatcaaagatcgcatgagttagacctgtttgattcattgctttcggaaactaagtatgtaggggaaatagggcttgatggtggacagggattta |
| | | aagaacattgggatattcaattgaaagtgttccgacacattctcaacagtgtaaatcgggctggtggcaagattatgactatccatagtcggggaagtgcatcagcggtgcttg |
| | | atgagattgaaaatatcgatggggtggcaatattgcattggttcactggaacacctaagcagcttgaaagggcaattgatttaggatgctggttctcagtggggcctgctatgct |
| | | cgatacaataaagggtaaggccttagttttgaaaatacccaaatcacgcattcttacagaaacagatgggccatttgctaagtttcgtaatgacccactaatgccatgggatagt |
| | | gggattgcagagaaacagttagccgcattatgggggattagtcagatggaggttaatgctcagctagttgataattttaaggtattatgtacatcataagaatgaaaaacttagat |
| | | atgcatttacagttcaattcatttttcgtcatcagttaattacacataaaattaaaagtaagaatatatctaccctgtgaatgagcaaggcggatttatatagtttgtaattagtttaaat |
| | | gtaagcagttcgtcagagtgcgtattccgctctattcgatcacggattggccgttatgaccc (SEQ ID NO: 380) |
| 23 | DUF4011- | gctatcctacctcagattactgggctgacctaatctatagatcaggttctctttatactttatgttagcgaaatactaagatgcttcttagtgacgacctcttgacggtagaggacgc |
| | helcase- | gtgcatagattttacaatcactgcctttcgccccctaacctaatccgcgaatgatgcatcctgaacttgcgcgccagttcttatactcgccgtcagagcaatcaaattgctgatgc |
| | Vsr- | tttctgcctgttcaaggcatctcctgtcgtcagcaatactgtgcatatttgattgatttcctcttaaggagaattagtttcatgggtattaaagcgcaggtgagtatcgcgcacaagc |
| | DUF3320 | tggggttcacatcacaccaaaatgcagttccgctgttacgtgagcttatcttgcataatgagtccgaagagacatttcaggatctgacactgcatctgaggaccgtgccagctg |
| | | tgctcgaagaaaaaaaatggaatatcgatcgcctgcttcccggtacttcacttgatatcagagatcgggatatcaaacttaatgctgaatggctagccgaactgactgaaagc |
| | | gtactctgcgaagtcacgctaagtttgcgccagggtgaggaagaactcttcattacccattacccgcttgaggcactggcgaaaaatgaatggggcggcagtgcaatgattg |
| | | aattgctcccttcatttattattcctaatgatccggctgtggatcgtgtactcaaggcaacctctgatgtccttcgccgtgcaggcaaggatgacgctcttaatggttatgaaagca |
| | | agtcgagaactcgtgtctgggaaattgcctcagctctctggactgctgtttgcaacctcaatatcagttatgcccttcccccagccagttttgaacgcaatggccagaaaattcg |
| | | cactccaggagccattctggaaggaaaagtcgcgacctgtctggatacaacattattatttgcttcagcactggaacagattggtctgaattcactgctaatgctcagtgaaggt |
| | | catgcgtttgctggtgtctggttacaaccgcaggaattttcgcagctagtgacagatgacgtctctgcggtgcgcaaacgtgtcgacctgaaagaaatggtcgtatttgagaca |
| | | actctcgcgaccagagctcacccgccttcatttactcaggcatctgatgaagcgttaaagcatcttaacgaggatgtttttcacgcagccattgattcccgtcgcgcgcgtatgc |
| | | agaaaattcggccactggctctggggggcactcgccttgaagaccagtcggatgcctgcgaggttattttgcatgggtttgaggaagccccctatatccccgatgttgatattg |
| | | atatcgagacaactggcgaaaaagaagccggggggcggctggtacagtggcaacgaaaacttctggacttaaccacccgtaaccgcctgttacacctgtctgaaagcgct |
| | | aaaggcattcgtttgatctgtgcgaatccgggccatcttgaagataaactggctgaaggcaaacgcattcgcattgtcccgctccctgatctcgaaagcggcggccgcgatg |
| | | ccgaactttatcagcagctcacaaatgagaacctgcaggaagaatacgctcagattgcgctggaacgcggtgaagtcgtctcctcaatggaaaaataccgcctcgagtcatc |
| | | cctgatcgacctctatcgaaaatcgaaaagtgatctcgaggaaggtggtgccaacactcttttcctcgctgttggcttccttaaatggaaaaaatctgctgatgaccccaaaagt |
| | | tactctgctccactgatactgctgccgattcaacttgaccgtaaaagtgcactttcgggcgtgaccatgcgtttgctggaagaagagccccgcttcaaccttacactgcttgagc |
| | | tgctgcataatgactttgctctgacaatcaacggcctcgatggtgatctacccaccgatgaaagtggtgttgatgtggatggtatctggaatatggtacggcgtgctgtacgcg |
| | | acatacccggtttcgaagtcacccgcgatgtcgtgattggcacattctcttttgccaaatatctgatgtggaaagatctcatcgaccgggcacctcagctgatgcaaagtgcgc |
| | | tggtaaagtatcttatcgaacgcggccaggaaaatgccgttctggataagagcggagaagtcatcaacgctcatgaactcgatgacaacatcaatacgcaggatcttttcttg |
| | | ccgttgcctgcagattcctcgcaaatcgccgctgttgtagcctctgcaaaaggcagggattttgttctggatggcccacccggtaccggtaagtcgcaaaccatagccaatat |
| | | gatcgcgcataaccttgcgctaggcaggcgcgtactttttgtcgctgaaaagaaagcggcgctggatgtggtctatcgtaggcttgaggcccagggactcggtgaattttgtc |
| | | tggaactgcactcgagcaaaacgtccaagatggattttctgaaacagctcgagcgggcatgggatgcgcgtgatctactaaccaccgaggagtggaaggaagaagcggc |
| | | caaggtgcagcacctgcgtgacaaactcaatgaggttgtccgtttgctccatcggcgctggcccaatggcttaacactccatcaggcaatgggcacagttatcagggatgca |
| | | agtagcgccacgccgcactttagctggcctgcatcgactttgcattcttctgcagagatgacacagttcagagagatagtaaaacgtctggagctgaaccgtgatgcatggaa |
| | | acagcacggcgatcattttgaactcatcgcgcaggctgactggaccaatggatggcagtcctctctcattgctgcagcaaactcattgcctgcaaccatcgatcaccttgaag |
| | | acgcgaccgaggcgttactgaaggcgacgggagttactctgctctctaccgagccggagagactgtcgcagttaacttcattctgtgaattattgtcggaagcttacggcatt |
| | | gatctgagtttcatgttcgcaccggatgccgcaagccgtatagagtcagcgaataaagccgttcacctcctgaaagagattgaagcgacaaaggctaatctgtcagttaccta |
| | | cccttgtaacagttggcagcacgttaatgtcccacagatcagaaacgcacttgacgtcgctgacaaaaaattctggttctttgcgaccagtgcccgcaagaaagtcattggtg |
| | | aagttatccgacaacactcgctaacgtcagcccccgacttatccgttgatctccccattgctgaaactctgcagacattgctgcaacgtctgaccgagcttaactctgctactgt |
| | | atctctgccgggatgggttggactggataccaacgttgcacagttgcagaccaccctgcaacttgccgaatctatccgcaattcgcttggtggtttcgcttcttcgccacagca |
| | | gttggccgagatccgcactgcggtaaaaaacctgattgttgatgccaatgaccttctcggttcgcagggcgttatctccgcactaacccggaaactgcgcacagcgatcgcc |
| | | gatttcaatgatgcacaggttagcttctgcaatctgataaaaccatctgaggataaaccatcgctcccggcactgcgtgactgcgcactcaatatcctgcaacatcagtccgct |
| | | cttaaagcctggagtgactggagccgtgtgcgtgaggaagcgatttcacatggcctgcaaccagtgatcaacgcgctggtccatcttgactcaggagacatcagcgcggca |
| | | gagatttttgaaactgcctattgccgctggtttgcatcgtggatgatcgattcagagccgctgctgcacaattttgtgccggctgagcacatgagtgatattgaggcttaccgtac |
| | | gcaaaccgatcgtctgtccaaactggcagtacgctacatccgtgcccgtttatgtggcgtcattcctgcaaaaaatgaggtcagcaagcagggtggttttgctctgcttaaaca |
| | | tgaactacagaaatcccgtcgtcataaaccggtacgtcagatggcagcagaaatgggagatgccatggccaaacttgccccctgcatgcttatgagtccgctttcagtcgcc |
| | | cagttcctgccctcggaccaggacttgtttgaccttgtgattttcgatgaagcatcgcagattgccccgtgggatgctatcggcaccatggcgcgtggcaaacaggtggtaat |
| | | cgctggcgatccccgccaaatgccgcctaccagcttttttaatcgtgcagccaatgacactgacgatgatactgaagaagatatggaaagcattctggatgagtgtcttgctgc |
| | | cggcctgtataaccacagcctgagctggcattaccggagccgtcatgaaagcctgattaccttctccaaccatcgctactatgacagtagcctgattacgttccccgcttcgga |
| | | aacaaagcaaagtgctgtccagtggtgcaaggttgcaggcgtctactctaaagggaaaggacgtcataatcaggccgaggcagaagcgatcgtcgctgaaacggtgaag |
| | | cgactgactgataaagagttcgttgcatcaggcagatcgataggcattatcacgctgaataccgaacagcaaaagctagtcagcgatctgctggaccgtgccagacagcaa |
| | | caccctgaaattgaacccttcttccagtctgaactggaagaacctgttgtggttaaaaacctcgaaacggttcagggggatgaacgcgatttgatcatactctgcatcgggtac |
| | | ggcccgactgaaccgggcgcaaatacaatgtcgatgaattttggaccgcttaatcgcgagggaggctggcgccgactgaatgttgccgtcacacgtgcgcggcaggaaat |
| | | gatggtcttcagctcgttcgatccttcctttatcgaccttaatcggaccaacgcccgcgcggttgctgacctcaaacactttattgagtttgcccagcgcggccctgtagctcttg |
| | | cccaggcagtacgtgggtctgtaggcggttatgactcaccgtttgaagaggcagtggcaaatggcctgagaagaaaaggctggcatgttgtcccgcaaattggcgtatccc |
| | | gtttccgtattgatttggggatcgttcatccggataagcctggcgactatcttgtcggtgttgaatgtgacggcgccacttaccatagcgcagcaacagcacgcgatcgcgata |
| | | aagtccggagctccatcctgcagggcctgggctggaaattactgcgcctctggtcaacagaatggtggattgataaagaaggcgcactcgacaggctggatgcagcaata |
| | | agtcgcctgctggaggactccagagcagcggaagccgcactgattgctgaagcagaaaaacaaaagcagattacgccagtcatcgctcccgtaaccaatgatgtcagtga |
| | | tgacatactggtttctgaaactacacctgtcgctaatgatgcggaaatatccgcgtcagtaacccctgtcatcccgcttactgccaaagtaagcgaagatgatggtaacactgg |
| | | gctgaggtatgcatctttagcttctcagaataacgacaagccagtgaatgtcggtaagtatgtcgttaacgatcttcaggaatggtgcgacaggacagatgcagaacaattcta |
| | | tatcgctgaatatgatgagacacttaaaaccctcattgaagcggtggtgacaagtgaatcaccggtcctggatacaacgcttgtgcaacgcatcgcacgtatacacggcttca |
| | | ctcgcgccggcagactgatacgtgaacgcgtaatggaaattgtggatcaacactatcaccttgcaaccgatcactcaggtgaagacttcgtctggctgtccgcagcgcaacg |
| | | tgctgactggaatgtgtttcgtttgccagccacggataacgacattcgtcaggttgacgcgatccccagtgaggaattacgcgcactggcgctgagtattgaaggtgacaata |
| | | agatacaggaaatgacccgctcgcttggcattaaacgcctgactagtcaggcaaaaaaaaggattgaatcagtacttgatgttgtttgaaggtcaaccgtgtggaaaacctctt |
| | | ttagagactaacagtctgaaatatagagtcttattcgatcatcttgagaccgaatgtattagagtcgatttctgacacctcttatcgtggttttctgcatcaccaacatcgaccagtt |
| | | gggcgtaatcaaggaggacgtctggaaaacgaatctatggtcactcccgtttttgcaacaccgattttgacaataagttggtttgcttgaatctattcggcatcagaatggaatttt |
| | | ttttccacgcctcgatgagttccgcgcctgatgaa (SEQ ID NO: 381) |
| 28 | ATPase + | ttaatgcaaacgcatcaggaagggcagacctagtcacatgtagaatacgatagcaataaaaaagtctaattagaatgcaaattgatgcaactctatgccctccaagaactcca |
| | protease | aacctgaaagatttatgtaaaacatagtgttcgtttcaccaaaatacatataaactacattaaaatagaaatttgtctcacctataagccatttagacaacagattaatgaggtttgta |
| | (ietAS) | tcacaaatgaccacaaacgagatactttcgcagcttatcagtcttggactcaaaggggataaagttgcttttgttcggcaggcttcgaaactcgcgcgttcctatgattctatggg |
| | | gctgcctgagcttgcttcagccattagaggtagtattcaagataaaaacacgtttaacttgcagaaagtatcacgcagtacatcacctatttttgaacgtcttgatacattacctgt |
| | | agataaagaaactaaatttgatttagcagacgtaactcaaccgtcttctgaaattcaactcccattgttgaaagatagcactctgaaaaaaattaaagaatttttgactttcactgaa |
| | | cgagctaaagaattaaaggatgccggtcttggcgtgacatcctctatgattttatatgggccaccaggttgtggtaaaaccttgacatcaaaatatattgcatcctgtctaaattta |
| | | ccgcttcttactgcaagatgtgactccttagtctcatcatatctggggtctacttctaaaaatatcaggcagctatttgagtatgcaagtaaagcaccatgtgttttatttctagatga |
| | | actagattctctagcaaaggctagagatgatcagcatgagttaggtgaactgaagagggtggtggtttctttattgcaaaatattgacaatctacctgaagaaacaatattgattg |
| | | ctgcaagtaatcatgaaaatcttctagatagcgcagtttggaggcgctttgagtatagaatatctattggattgcctgattttgaagtcagaaaacaactatttgaacaatattcaaa |
| | | cataaaagctacatatgacgattttgttgatgaccttgcggaaatatcatcagggctaaactgctcatttatagaacaatgctgcttaagatctgagcgacatgctctggtttacaa |
| | | taataaacaaatcgatacccgatttttagtcgaggctatcttagaagcgaagggagttacatttgatgaagaagataatttacttataaagattgtgaccactctcagagaataca |
| | | atcccaaaagatttacaatacgaaagatagcaaaaatactagggctttcaaatgctaaagtgtcaaggctaactaagaactatagagagatattatgagtaacaaagaaagac |
| | | caataaaaataattgaggcgacacctcaagattttactgaaaaaacatataatttcggaaagaaacaacctatccgaacagtaacaactagtctaaaaaatagactcaaacaa |
| | | gaagtcgatgacgttaaaaattttttccagagctcatttaaaaaatggcccaatataccggcggtggctagagttactcttcatgaaaaagctcttgctaagtcacatcgcccatc |
| | | aagcctattaggtgataatacatgtcccgtaataggcagtgataattttggagaattacttataagtgttactgaaaaagggttagcacaacttcgcaaaaaaattgaaaatagca |
| | | ctaattctcataatgggacagtacatattgctgtaattgaaaagatcgaaccttttagtcttaaccatgatgttatagataaaaataaatcagatagttttcttctgaaactctttgacc |
| | | ataaagatagaacaactaaccgcagtatcgacaaagaattaatggaatttgcagatgaactaggaatacaaaaacccaaaaagtatgatatcagttcagatttgagtatatatg |
| | | aagtaaaagggaatgataacatcgcccaactggcaagttttattggcatacgaaaattagaacctatgccaacatttggtcttactcatacagtatcgcaatatattcctgctgaa |
| | | actctagacctagatgattttcccttacctcaagaggataaacattatccactactcggaattatagatagcggagtcgatcccaataacaacatacttaggccatggatttggga |
| | | tagtttagatttagtaaaaggagaacacgactattctcatgggaacatggttgcaagtttagcaattaatggaagatggttgaataactatgctggttttcctcaatgccaagctga |
| | | aattgttgatgttgcagcctttcccaaagatggtacgctcaaattgccacaattaatgaaagctatccgagaggctgtgaccacctatccagaagtacgtgtatggaatctgtca |
| | | ttaggttgtcaatccccatgttctgaagacagcttctctgaattggggcattttttaaatgcacttcatgatgagcatgattgtcttttcgtcgtagcatccggcaactacatttatgat |
| | | cctcaacgaacctggcctcctcaagaattaggtgggcatgacagaatatcagcccccgcagattctgttcgttcattaactgttggctcagttgcccatttagaatcgtctgactc |
| | | tgtggtcaaaagatttgaaccttcatctttttctagaagaggtcccggcccagcctttatacccaaaccagagataaatcactttggaggtaattgtgacagtaaattaaactgtg |
| | | aacataccggaatcatagctattggcgaggacaatgctctttgcgaaagtattggcacaagtttatcagcaccgttaatctcaagtttagcggcatcactgtggcatgaactaga |
| | | tgttaatggttctatttcaccatcgcctgaacgtatcaaggcactattaattcattctgcgttaaaaaactcaccagccaaaacggagcattatgcgtttaattatcaaggatttgga |
| | | cgcccaagcgatcatataaatgatattattggttgcaataaaaatgagattacatttctatttgaaatagatacccgagaaggtattgaattcagtagaacgccatttgtaatacca |
| | | cagtcattacgtactgaggatggaaaattcacaggtgaaattattatgacactcgtttattctccaccgcttgattatgactacccatctgaatattgccgttctaatgtggatgtgtc |
| | | attcgggacttacacttatgatccagttaacgctaaatggatacatagcggaaaaattccacaaataaaagaaaagagtgaattatttgaaaaggtactgatagaaaatggcttc |
| | | aaatggtctccagtcaaagtttatagaaaacaatttccgcaaggtataaatggggagcaatggagacttaaacttgatgttcagagacgagcagagcaagagcctctatcttc |
| | | acctcaacgtgctgtattggctattacgttaagatctcttgccaattctactacagtctacaacgaagccgaggttgaaataaataatcttggttggaaagaaactgatattgttgtt |
| | | cgtgaacaaccaaaaatcaggattcgtcaaaaataagcattatggtcaccttttataggtgaccattta (SEQ ID NO: 382) |
| 28 | ATPase + | gggacactcaggttacataacaatgagtgatacagttcacgtagtgaaggtactatgcctaggtgtttgattacactttgatcattgatgatacgctcatgaaggtattactttcct |
| | protease | gtaatgagcaggtaggtaacgatgtcgaactaaatgaatttatagtaaactttgcaacaagagaacaagggagtatgaggggttatggctactgcagagcagatcaaagcttt |
| | (ietAS) | attgaaaagccacgttgatcgtgatgatcagcgtttcttttctattgctttgcaggtggcagctaaggaagcaaggcaaggtcatcataagcttgctaatgatataaaaaacttag |
| | | ttgataaaaatcagaaaacaacgagttctgtaggtttagttgaaaaacgacttacaccatttgttaagcagcctgatggtgatcttaaggggttacttgagcaaacgaacaagcc |
| | | agtacatcttcaagatctggtgatttctggaagcgttagggaaagattgaatcaggttctgcttgaacaaaaacagaaagataaactttctgagtttgggcttattccaagaagaa |
| | | aaattcttttcactggtcctcccggtactggtaagacaatgtccgcatcagtcattgctacagagttaaagctaccactttatacagtcgtcttagataatctaatcactcgctatat |
| | | gggtgaaactgcagctaagctgcgtttaatttttgaccacatacggcaaacaagagctgtatatttttttgacgagttcgatgctataggaactcagcgtggcgctcagaatgac |
| | | gttggagaaattcgtagggtcttaaattcttttttaatgtttgtagagcaggatgattctgagagcatagttttagctgcaaccaatcatccagagcttttagatcgcgccttatatag |
| | | acgatttgacgatattataccgttcacaaggcctgaggataatctaatcaggaatcttattgaacagagactcgctgtctttgacctcggtaatttattttggagtgagatcattgat |
| | | agtgcttcaggtctaagtgcagcggagatcacgcgagcaagtgaagatgctgccaaagaatcagtgctttataatgcaaacaatattacaaccgatttgttagtaaaggctata |
| | | aagcgtaggcaagaaagtagacaataagggatgaaatgactaccaacaagaggcatattttattaaacggctatgtttcccccgaaaactatcgctctaggagcaatggtcgt |
| | | agtccccaagtcccagctcgtgatcgagcggtacatggtatatcattactaaatcagtatagccgtatattgaatcattatgatgaaagaccgaggcttccccctgttactgatga |
| | | aaaagggatttatgttaggctaatcagttttgaacaatgcgatcttcctatagataaaatcgataatacttatttcaagctttgttctttagttaaatcaaataatcgtgaaactgcgatt |
| | | atatacattaatgaaaatgacagaactaaattcactaaaaaaataaatgactatttgaatccatcgaaggatggtatcgagttccctagaaatcatttgttaattgatagcatacaa |
| | | aatatcgagttagcagatataacttctttctggacagataaaaaagatcttattccggatgatcacggtgttgaaaagtggtttgagctttggcttaagggtaataaggaggatgt |
| | | gctaaatattgctcggcgtttatgcgaaagaattaatggaaggctcgggaatacttctattaattttttcgatactactgttgttcttatccgtacgagtctatcgagattaaaagtttg |
| | | tcctgaattaatatctaatttaaaagagataagatcagcgagggatgatatatcagttatagttaattccttacctacagaacagcatcagtgggcagaaaatgttgctgcaagaa |
| | | ttacgcgtaacaatgaagctgatgtttctgtttgtatattagatacaggtgttaactacaataatccactattatctagatttactaactcatcactggcagctgcttgggacatatctt |
| | | ggccacttttcgatgattataatcaaaggccttataatgaccacggttccagacaagcaggactatgtgtttatggagatttcctgtctgttttattgaacgatcaggacatttcgat |
| | | tccgtacaatatcgaatcaggaaggatactacctccaagagctactaatgatcctaatctttatggagctattactacaggaacgtcaagtcgtctggagctggaaaacccgaa |
| | | ctggcgcagagtttattcgcttgctgtgacagcagagcctaatactcttggaggccaaccgtcctcatggtctgcagagattgacaagtttagttttggtttagaggatgatatcc |
| | | gcagattatttataatttctgcgggtaactctcaacctacaaatttagaattagattattgggattcagtgactcttgctgaaattgaagatcctgctcaatcttggaatgcattaactg |
| | | taggggcgtatactgataaaacaacccatacagaccgcgaatatgatggttggtctcctttcgctatgtcagaagatattgcaccgtcatctcggtcatcggtatcctggggatg |
| | | gaaaaagcatgccccatataagccagatttagtagaggaaggcggaaacaaacttatatcacctagccgtgatgaaatcacaaatacaattgaattatctttgctcacaacctc |
| | | tggcagggcaacaaatcaattgtttgaagttaattcagatactagcgcagcctgtgctctagtatcaaaacatgctgctatgctaatggctcagtacccagaatattggcctgaa |
| | | actattaggggattacttgttcatacagcaagatggactagtcgtatgcacgaacgatatagaacagaacgtgcacaggggacaccaaaatcggctaaagaaagcttattaa |
| | | ggatggttggttatggagtacctaatttaaatcgagcaatgcatagtgcggaaaatgcacttacattaatatctcagtcggaaatcaccccatttaaaagagatggttctactgat |
| | | cctacattgaatgaaatgcatctgttttcactcccttggcccgtagaagctcttcgcttactaccaccagaaacaaatgttattttaagaatcacattgtcgtattttattgaacctaat |
| | | ccaagtcaaaaaggattcagacgacaatattcgtatcaatctcatggattgagatttgcagttattagacctaatcagacccttgaaaatttccgtgcttcgataaaccgtaatgc |
| | | gaataatgaagaatacaatggacctgaaggagatgcgtcaggatggtttctggggcctcaactcagagttagaggttcattacactcagatgcttggaaaggcagtgctgca |
| | | gatttaacagagatgaatactatcgctgtctatcctgttggtggatggtggaaatatcgtactgcgcaggatcgctatattaacaatgttaaatatagtttattggttagcatagatg |
| | | taccagatgagaacattgatatttacagtgagattcaaaacattattcaaattgataatcaaatagatattgaacattaaggttttatgcctaaggtttaatgagtttgaaatgaaaaa |
| | | tcctttactaattggctgggtcgatgataaagacctggccatctttttatacggaaatgatttatgttttattttactaaatttatattagaaccatcgtgcagattgtgataattccttcat |
| | | actgattttttacctattatagttgatttttgttgcttgatatctctctttaatacaacggcgtagtac (SEQ ID NO: 383) |
| 30 | Retron- | tctatctaaaagtatacatatagtatttcaatgaaggttatattatattttgtggctgttttctaattttatcaataagattattgcaaaaggctgataaatataatagctttattatatcgga |
| | protease | ggagttgatttaactttcctatactatctgtataggctaataccaatggcaattttgccctcaaattggtctccttaatgtttatcaacgtgttatacggtagtgataaaacctcctccg |
| | | atatttttctcatgaattgggatattttaaatatgttttgctcagtaaccaagttgcatgaatgtaaaaatgttgaacaattatactattttttaggatgtgaagaggctgaaattagtag |
| | | gtttttatatagtggagtaattaaataccgctctttttccatacttaaaaaaaatggtaattttagaaatataagagcacctgtaaagtatttaaaagaaattcagtataagataaagg |
| | | atgagctcgaaaaatattataccccgaaatcatgtactcatggttttatagctggaaggaatataatcacaaatgcgaaacctcatataagaaaagaatttattttaaatatagattt |
| | | aaaggatttttttgattcaattaattttggacgagttagtcgtttatttcaaagccaacctctaaacttgccagagaatgttgcccatgttttggcacatatttgttgctataatagagcc |
| | | ttacctcaaggtgctcccacatccccaattatatctaatatgatatcttatcgtttagacagacaattgaaggagttggcaagaaataatgcgtgtacttataccagatatgcagat |
| | | gatataactttttcttttactaaaactaaaaagtatcttccaaaatcaattgtttctttaagtaaagataataacattatactaggccatgaattaaaaaaggtaattgaagataattggt |
| | | ttgaaataaatgaaggaaaagtaaggttacaacataaaacacaaagacaatcagtaacaaatattacggttaacactaaaattaatataagtagaaaatttaaaaaacaaacttc |
| | | agctatggttaatgcattatttaaatatggagcatctaaagctgaaagagaatattttagtaagtatcacaagggttatatagcagaaaggcaatataataagattaaagaaaaac |
| | | caggtttattatttacacaaaaagtaagaggaaggttgaattatatccgattagtttgtggtaagaataatgaaagctggagaaagctcatgtataaatatactgtggcaatagga |
| | | caacctaatgaggagtacaatagaacattgtgggatattgctggtgattcaacgttcattctttggtcgaattcctcacaaggaagtggtttttttcttgaaaatattggtttagttac |
| | | aaatgagcatgtaatcgaaggaatagaaaacagcaatattaataatgatctaataatactttggttaccaaatgaaagaaaagaatatattgagttacacttagcttggaaagatg |
| | | ataatactgatttagctgtaattacttctaatatatcttttcttgacataaagcctttacaagtagagccagttcctatttatgatataggaacagaagtatatgcagttgggtatcctaa |
| | | ttatgacgccagaggctcaattggaaaacctactattattacagcaaaaataacgagtataattactcgagaaaggcaagaaagaatcgttatagaccaaccaatagtacatg |
| | | ggcatagtggtggggtcgttttaaatgctgatggacgtgtaataggcattgttgcaaatggaaatgccgagggggaattaagagtagttcctaatgcttttattcctattgaaatat |
| | | tattaaatgagcacaagttacgaactaaatcataaaattattattcttaaaataattaaatattttttaaaaccactagtttgataactagcggttttttatttttggagtacat (SEQ |
| | | ID NO: 384) |
| 30 | Retron- | ctttaaaatgtttcatacagcatacttgtataaaaaaaactttatgctataaagacataagtggcggcctttgagtttaactttcctacgactatctgcgtaggtcatttttcaacggca |
| | protease | gttttgcactctaagtttgccgataagtttgtcgcgcagctggcaatagagaaaacatggccgccactcttccatataaggatttttatgccctcattttcattaaaagaatgtaatg |
| | | acgtttggaaattatgtgatttactgggagttaacttcgaaaatctatctaaaaaagtatatccaagtaataataggttatatagatgtttctttattccaaagaaatctggtggactaa |
| | | gagaaatatactgccctattaaatcacttaagaacttacaaaagaaaataaaaatagagctagaaaaagaaataaaatacagatcgcctgcacatggatttattaaagggaaa |
| | | agtataataacaaatgctgaacaacatataggaaaaactatagtacttaacttagacctcgaagattttttcaaaaatatacattttggcagaataaaaaaattatttgaatcaagcc |
| | | cattaaatttaaaacactctgtatcaactttccttgctcatatctgttgtagaggtggtgtattaatagctggttcgccaacatctccgattatatcaaatatgatttgttataaattagat |
| | | ggtcaacttcaacgtttagctaaaaaaaaccactgtacatacaccagatatgcagatgatataacattctcttttacttgctcagaaagaaagttgccgagagggatcgtacatat |
| | | agatgaaagttcattattaggttttaaattaggcgatgagttatctgaaattatttcaagtaacaacttcactctaaatgaatctaaaataagattaagtcgaaaatcacaacgtcaa |
| | | gaagtaacgggtttaatagtaaattcaaaagtaaacgtaaaaagagacttcattcgcagaacatcatctatgattcatgctctaaaaattcatggtgctgaagacgcagaaaaa |
| | | gaacattatttaaaatataaaaaaacttatataccagaaagacaaaataaaagacaaaaggataaacctggagatctatacacaaaagtaatcaaagggagactaaactatctt |
| | | agaatggttagaggtgaggattgtaacttgtggcgtaaacttatgtatgattttactgttgcaatgaagaatccagatgagtcttataaacgaacatggttagacgatgcggcag |
| | | agtctactgtgatatttaacacttacgatgggtgcggcagtggttttttaataaatcatgatatcaaaaaatatcccaatggactcattattactaattatcacgtgattcctgagata |
| | | aatagtgataatatttcaaacattgaagttcatacatggatgaatccttctaaaggatttttattacttaaatttgtagcttcaagtaaagacttagatattgctatattaactgcggaca |
| | | taccatttccagttagtaagtttttggttgtaaattcatgtcctaactatagacctggaattaaaattcataccataggatatccagattattcatctggagaggatccaacttttatatc |
| | | tacaaaaattaaaggtaaaactacatatcatggtcaattgagatatcagatcatagatgaaataaaacatgggaatagcggaggccctgtctttgattcagatagaaaagtcata |
| | | ggcattgtgtctaatggaaacgaaaaaggtgcaccaaaaaacaataagagtagcttcataccaatcgagaccttgcttgattttataaattgtcaaaagtaaatgttttaaaaaaa |
| | | ccatacattgataactatatttttacacagtaaaaaacaccataatcttatatggatatcagatta (SEQ ID NO: 385) |
| 30 | Retron- | aagaaaaaggaatcttctaaattaatgaaactataattatacgaatcagtaataccacagttattgacatattttgtaataagctttatttttactaaagcacagtacatcatacaaatt |
| | protease | taattttctactgacttatcagcggtagccataaacgtgtatcttctgcctcagctatcctacagtttcttgtggattgtcgtcattgcaaaagagaaaactagatgatgtattgtgct |
| | | cccctttttaaaggactcgcatacaatgtttgacccattcaaagtagcgccgccaaaattgaaactacatcaatgtgtagacgttcatgagctttctgcaatattaggaacgaact |
| | | acaatcagttatcaaaattaatatatcctaccactcaaaattcttattattgtttcagtattgataaaatgaacgggaacaagcgagttataaatgcacccaaaaataaattaaagtc |
| | | gatacaaagacgattagcatatttacttaatgagtattatcctgtcagggatgttgctcatggttttattaaaaataaaagtattgtgtcaaatgcagaacagcatgttcttaaaaact |
| | | gcgtattcaatatagatttagaaaacttctttggtcagatccatttcgggcgtatacgtaatttattattttcaccgccatttaacttttcaacttcggtatcaacagtaatttcacatattt |
| | | gctgtagtgatggttttcttcctcaaggtgcaccaacatctcctataatatctaatttaatatgttataaattagataatgaacttaggcgattggccgtttatcataaatgtacttatac |
| | | aagatatgtagatgatataacattctcttttacatgcaaagcaaatagaataccatcacaaatagttgtatcttcaggaaatacggtaacgccaggtaatgagataaatgcaataa |
| | | taacaaggaatggtttctctataaacgacaaaaaaaccagactgcaacaaaagaatgaaaggcaaatagttactgggatagtggtaaataaacggacaaatgttcaacgga |
| | | gttttgtccgaaaaacaaactcaatgctgtatgcatgggaaaaatttggagctatcttagctgaaaaggattactttgataaatacaatagcaagattaaaactataaaactaaaa |
| | | gatttcattgataatccgggagagttatttaagagtatcgtaaaaggaaggataaactatataaaaatggttagagggaaagatgatgtaatatatagaaaattcgcccatagga |
| | | tatcttgtttattcggcaagtttgataataggtatcttaaaacaccgtatgattttgctattgaatctacatttgtactcgaaaatagatgtgatgactcacaaggtactgcatttttacta |
| | | gagagaatagggttggttacaaaccatcatgtcgtagaagatatctgtgatatcacagatgagtttattgacttattcttatggaatgaaataggcaatattcgaaagacaaaattc |
| | | ataatgtcaaacaaactgtttgatattgccgttttcgaaagaacatccgacttcgacaatataacaccattaaaaattggtgatgatagtggaataaaaaatggtactgttattaca |
| | | gtaattggtttcccacaatattctcctggtgaaagcgcttatgtgaatacaggaaaggtaattcaatcgaaaactatgtatggtaataaattttggcttattgatatacctgttattcat |
| | | ggaaatagtggtgggccagtattaaatgacaaatttgaagttataggtattgctagcatcggtacagcgaagaacgatagttcatctaaacttcatgggttcattcccatatcgac |
| | | tttattaagatatacgggtgaagataagccttaatctctctttctctaagtgatttttaaagcgcctacagtccatactgtctggcgtttttttttgttaccggtcatacgtgccattctga |
| | | tgctgagaatatgacattgggcat (SEQ ID NO: 386) |
| 30 | Retron- | ttacattactatataatatgcaattaaaatgaataatttatactattgacatattttgtaatacgctatattttttaacggcacagcgcattttatcacaatttaactttctactgactatctg |
| | protease | cggtagccataaacatgtaacttctgcatcggccgactttccgtatctcgcatgtttgccgaatttgcaaaagagaaaatagataaagtgcactgtgccctatttaaaggaatgat |
| | | aataaaatgtttaatccaaccaatatattaccaccaaaaataaaattaaataaatgtggtgatgtacatatattagctgcgttatttaatttaacttatgaagatctatctaaattaattta |
| | | tccaactccaaatagatcctattatcaatttgctatcgataaaaaaaatggtagtaaacgggtgattagcgctcccaaaaagaaattaaaaatcgttcaaaaaaagatagcagat |
| | | gaattacttacactttatcctattcgtgatgtttctcatggttttattaaaggaaaaagtattgtttctaatgcggaaaaacatgttcttaaaagttgcgtacttaatatagatctcgaag |
| | | atttctttggaagtatacatttcggaagagtaagaaatttgttaacttcaccttcatttaatatacccttacctgtagcaacagtgatttccaatatatgttgttataacggatccattcc |
| | | acaaggagcacctacatctcctattatttccaatttaatatgttataagttagataatgaattacgacaactcgctggtaaatataattgcacctatacgagatatgtcgatgatataa |
| | | cattctcattcacatataaagccaaaagaataccatatcaactagttacctctgatgccaacataataaatataggagttgaattagaggaaataataactagaaatggtttttcaa |
| | | ttaacaaaaacaaaactagattacagagtaaaaatgaaagacaaactgtcacaggaatagttgtaaataagaaaactaatttacagcgaaaattcatacggcaaacctcatcc |
| | | atgttgtatgcatgggaaaaacatggcgtagtagctgctgaaaatgaacactttgttaaatataacaaaaaaaataagctaataaaattaagggatttcgtagataaaccagga |
| | | gagttgttcaaaagaatagtaaaaggtcgaataaattatataaaaatggttagaggtgaagacgatataatatatcgtaaatttgctcacagaatatcttgtttatttggcaatgtaa |
| | | ataatagatatttgaaaactccatctgattttgctattgattcgatttttatcttagaaaatgaggtggatatatcacaaggtacagctttcctcttagaggatgttggtattgtaactaa |
| | | ttatcatgttgttccaagtatagatgaatataatgatattgacttatctctttttcgatataatgaattggataataaaagaaaagtaaagttcataatgtcaaataagttatacgacttg |
| | | gcgatattcgatactaatggcaattttgatgatataaagaaattttccataggggatgattctaatttaaaggtaggttcagaaatatctgttattggcttcccacaatataccacgg |
| | | gagagtacccttatataaataccggtaaaatagtccaatctaaagctcttttcaataataaaatctggcttgttgatatacctattattcatggaaatagtggtggtccagtttttaatg |
| | | agaaatttgaaattattggcgttgcctcaaatgggacggagagaaatgatcagtcatcaaagttacatggcttcataccaatatcaacactaataaaatttattagcagtaaatga |
| | | ttttaatattaaagtgataagcgcccctgttacgcacacagagaggcgcttttttatttcacctctcatgatgaatcgtttcgagccaaaaaggcagagt (SEQ ID NO: |
| | | 387) |
| 31 | RT- | agggatacgccacagcaagaaatagtttacttattcctcattttgtcgactaaaaatcgacattaaacaaaaaattcaaacttaatcactttcgggaaaaatgtgacaaatatatgc |
| | nitrilase | tcggactggttgcggggagcgtgtaacatggatacaaatcaaaattattgccagcctcactgatggattactggtgtcaagagccccccttcgggcatgaaacggctggcta |
| | (UG5) | attctgtacagactgtaatctaaggacgataacgcatgacatatcaggcaattttcactggctgggatgatctgacgattgaagaccttctggtcgcttaccggaaagcaaaag |
| | | ccgatagcttctttgagaatacatttcctgttgctatcaaatttgccgagtatgagcaggaattacttgaaaacctgcaaaaactcttagatcttttgcagagcgaagatggattca |
| | | gtagcaataagaagttgattggcaaatttcgtttgttaccgaaaaaattaaccacaaagaaaaaacatgaatcccaaaatggacacgtccacttttctaatcctaaacgagcag |
| | | ccgaccatttatttaataattttgatctgataccagagtttcgtattattggtgacttcccggttgatagtcacattatctctgcactatggattaacatggtcgggcataaatttgatgc |
| | | cagcttagataactgttgctatggcgcgcggctaaagcgtattcgtaatgatgaattatttagcaatgagcaggataatccattccatatcagtgccgtgggttcttttagccccta |
| | | cttccagccctaccaaaaatggcgtggtgatggcttaaaagctatacgtgacgagttggaaaaagatcgtgacattatcgccgcctcactggatttaaaaagttactatcatttta |
| | | ttgatccactggctataacctctgatgatctctataacacactaaacataaaactgactgaggatgaaaaagcgtttactgcacagttagcagtattcttaaagcactggtctgac |
| | | ggcgcagcggcatttggaaagaaaatagcgtacaaaacacctgttattaatggtggtctggtcattggattaacagccagtcggatcatttcaaatatattgctacaccattggg |
| | | ataaattagtcattgaaaaactatcaccaattcactacggtcgttatgtcgatgatatgttccttgtaatacgcgatacagggacaattactaataatcacgaatttatgttattgctg |
| | | caagataggcttggcaatgattgcgtttatttgaaaaacgagcaaaaacaaatatggcaaatacagcagggcgagcatttccagggtaagaccaccatccagttacaatccg |
| | | ataagcaaaaacttttcgtgcttcaagggagggctggaatagacctgctcgacagtatcgaaaaggagatctacgagctttctagtgaacaccgcttgatgccttcaccggat |
| | | caactggaacactccaccgcagctaaagtcctttccgctgccggtagtgtaggtgaaaatgccgatactctgcgccgtgcggatggattaaccattcgtcgtttgggctggtc |
| | | actgcaattacgctacgttgaaacactggcacgagatctgcctccaagtgaatggaaagaacagcgggaagagttttatcagtttgcctacaaccatattcttagggctgataa |
| | | tctatttgcacattttagttatctgccaaggctgcttggctttgctatcagtatgaatgaatggcagcacgcggaaaaaattgtacttaaagcttacgaatccatcaacctgttggc |
| | | atcggtgattacttcaggtaaggaagtgaatataaatggttgcaaaactcgagcagtaaatgatctttggcgctgtataaaaggcacattaagctggctatttgttgatgcagcg |
| | | acacgatattacagtcctgacagattatttcttgataaacgttcaaagaaagaagagtgccttgcggatacattttttaatcatatttcacaaagtctgacgaatctaaaggatttac |
| | | tggatcttcgctttgattcagcagatttttatttaaaagcgccattggtagctcgagctgatttagcaaaggaaccttataaacagatcgtaaagagtcagtcggcagaaaaactt |
| | | gttaatcagcgtgatagtaaaaaagaagttaaaatactgaaattaatgagcgactcatcgcttattgatattgacgttattaagctatttttgaaatcaaccaagaatacccgactg |
| | | gaaaaagtggctaaaggaaatcgtaagaacgaaagttacctaccttacattttccctacacgtcctttaacacccgctgaaatatcagaactggcccccgaatgtgttggatta |
| | | ccctccacatccgacaaaaaaccagatgagagaccgtccaccatttgggcaaaatatactcaagcattacgcggagtatggatcaaaccgacgttgctagcatcggagcag |
| | | gactcagatgaagcgacaaaaaaagctcggcctaagaaattcattcatattggcacagacaggaaacataaagttgtcgttgcgctaaccagcattaaaacagaggaggac |
| | | gactgggctaaaatggcctgcaataaatctaacttgtcccgttcaaggtaccagcggatttctgaactggttaatgcaacattgaaactatctcctaaacctgattatgttttattcc |
| | | ctgagctttcaatcccgttacgctgggttaacagtattgctgatcgtttgagttcggcgggtatcagtctaattgcgggaacagaataccgccacttagacgataatcaactgaa |
| | | gagtgaggccgtacttgtcctttcagataacagactcggctatccagcgagtgtcaaaatatggcaacccaagctggaacccgccgtaggtgaagatgaggcattattttcaa |
| | | tttatggtaagtcttgggattcgacacttaatgttaaacaacgtaagccggtatatattcatcacggcgtcaattttggcgttatgatttgctctgaactccagaatagtaaagcgag |
| | | gatccgttttcagggcgcactcgatgcattaatggtattgagctggaataaagatctagatacgtttgcatcgttgattgaatcagcagcgctggatattcatgcctatactatttta |
| | | gtgaataaccgaaaatacggcgatagtcgcgtacgttccccggcaaaagaaccctttatgcgtgatattgctcgtgtgaagggcggtgataatgactttgtggtcgctgcaac |
| | | gctggatatcgactcgttaagggcatttcagagcagggcaaaacgctggcctaaaggcggcgataaattcaaaccgttacctgaaggattccagttggcaaagaaccgcaa |
| | | aaagctaccgccaaaataagaaactgattttcgctattaataatcagggtatttttgcgtgagatgttggtaaacatgatgtagcccttgccactcatgaccaatcgcagtatcttt |
| | | ctcccgcgcctgcaaaatcaggcgtcgggattagcctcctgaagaaatcttatcggcgacacatgacgcgccagcgtctttttttgtgttgttcgcacggttacatc (SEQ |
| | | ID NO: 388) |
| 31 | RT- | ttttcaaaggagtttcgctttccaaatatacaagaaatcattatttctaaaggtatctataagtggatgattcgttttattggaacagttgcattctcgttaattaaagcggctgcttccg |
| | nitrilase | accggcgaatggtcattcagaagctgagaatgtggttattttttaaagaggaattggcatgattattagccttgaagagcttggccttgcctaccgaaaagcaaaagtcgatctg |
| | (UG5) | tactattcatcccatgtttcgctggaagcaattgcgtcttacgaagagtccctacatacgaatctgacggttctgcaggaaaaaatacaaggtgacgacgaatcatgggtggaa |
| | | gagaatgagttcactggcaactggtttctggccacaaaatctgtagacatgtcttgctgggaacagcagcgagaaccgcaagctaacggtctcatattttcctcacctgctgaa |
| | | aagtgggcatatgcttgcaacccaatggctgataaaaacgaacaaaaaaaaatcaaagccgagtttcgagtaatggctcaatgcagtctggattttcatgttctctcgactcttt |
| | | ggatgttaaaagtcgggcatctttttgatgccaaattatctacctgtgcttacggtaaccgcctgcgccgtactctagatggaaaagacatcaatgcactttcaattggttcttttca |
| | | accttacctcagaccttttcgtgattggcgtgacaatggcattaacgccatgcggagcgcgctaagtgaaagcaaaaaaatcgtggcactcactgctgatgttagttctttctat |
| | | cacgaactgaatcccgggtttatgcttgatccaaccttcgtcaaagatattttggagttggaactcactgctgaacaaagcaagcttaatcgattattcattaatgcgttaaaagca |
| | | tgggcaattgagactccgttgaagaaagggttaccagtaggtctccctgcttcagctgttgttgccaacgtagccctgatcgagctggatcgcgttattgagcagcaagtcgc |
| | | acctatatattacggacggtatgtagatgacatcattctggtcatggaaaatggtgcgaatttccgttccatggcagagctatggcaatggttgttcgcccgttcttccggcaaac |
| | | tggactgggtaaagggcgaggaaaacaaacagatcagttttcaaccaaactacctgcatgacagccagattcgttttgcaaatgcgaagaataaagtgtttatccttgcgggt |
| | | gactccggaaaaaccttagtggaagctattgctcatcagatttatgaacgagccagcgagtggcgagccatgcctcggttaccgcattcctcgaacaatgttggaactgattt |
| | | gcttgctgcaactcaaagtaatggcgaagtcgctgacaatttgcgtaaagcagatgcactgactatgcgtagggctggttttgccatcaaactacgcgactttgaagcctatga |
| | | gcgtgacctgcaaccgggcacatggaaaggccatcgccaggcattttttcgggcatttattgatcatgttgtggtgctgccacaattctttgatttatcagtctacctaccccgag |
| | | tgatccgactggccacggcctgtgaggactttgtcgaactgcgcaaacttatcttagcgctcgagaatatttgcgatgaagttcgagaaaattgcctccttaccatcaaggcgt |
| | | gtcctgatgatcacctcccttttgaagcagagattattggcaaatggagggctcagctttttagcagtgtgcttgaagctatcgttgcggcatttcctccgcgtatttccaaggtgg |
| | | gtaagcaaacctggaatgaccatttaaaaaactggcacgcccggtgtgggctagacattcaatattcgggtcgtgatttttcattaaagggctaccaagaacagcaggcgag |
| | | attattctctttcgacttagcgcacatgccattccgctttattggtctaccaaaagagatgattgctcaacggggcatacccgctccgaaaacagtagcccactgtgcggaagc |
| | | agcagaattactgcctgatattgtcgttttgggtaatcaggttgtagcaaaatggtgcaaatttaaaatcattccacatggactgctatttgccacccggcctttcagcctgccgg |
| | | aactctttatcctaaacaatgaggcttatacagcttcagctcagcaagaaatgcgagctattattttcgctgttcgcggttttgtactcggtaataaaacaccttgtgtcgataaaca |
| | | aggcatattgcaaatccctgacggccaatctgctggaaaatatggggttgccatatctagctggaaaacgtccatgtcaagctggactgcggcggtcatgcgttcagccgat |
| | | ccggatgcaaaccgttacgctcgcttatgtcgcttgcttgatggtgtgatagcccaaccacataacagtcgttacttaattctgccggagctctcactccctgcgcactggtttatt |
| | | agaattgcccgtaagttacaaggtcgcgggatttcacttgtcaccggcattgaatatttacatgccagtaaagcaagagtacgcaatcaggtatgggcttccttgtctcatgatg |
| | | gattgggttttccttcactaatgatttaccgtcaggacaaacaacgcccagcactgcatgaagagcaggaattacaacgaatagcagggctagaaatgaaaccagaaaaga |
| | | aatggacaacgcctcccatcattcaacacggtgattttcgtttttccttgttgatttgtagtgagctgaccaatattagttatcgcgcagcgctgcgtggcaacgttgacgcgctgt |
| | | ttgtgccagaatggaatcaggatactgaaactttcaatgccttggtcgagtctgctgcgctagatatccatgcttacatcatccaatgcaatgaccgccagtatggcgatagcc |
| | | gcatccgaggccctttcaaagatagctggaagcgtgatgtattgcgagtcaaaggtggtattacagattattgtgtaataggcgaaattgacgtacattctttacgacaatttcaa |
| | | agtagctatcgttctcctggtaaaccctttaagccggttccggatggatttgagatagagcactctcgaaaaatgttgccagaagcataagtaaaattggaaaaaaatatcgatg |
| | | caggttattaaagatgaggcaacatgccatagtcaatcataacctgcagatgtaatttgaaactgcatgttgagaattacggatttatttgtgtattcaccctcgcataaaaatgaa |
| | | gtagctttcatattccacactactgataccccctgaaaatatataactaaaaaaaacaattttaaaacatgaggtaggaatagcaatctgactgtgatgtagttatttttttgatgaag |
| | | ataattaggtgctcgttgttc (SEQ ID NO: 389) |
| 32 | TOPRIM- | atgccccgtatcaacgttgagaaactgctgcttgagatcgaaatcgacaaggtggcagagcgattgggtatggcgcttaggagcgaatcagctacgcgcaagctcacgctg |
| | RT- | tgcccgttccatgacgataaaactccttcccttctaattgatacgagcagagataattctggacagcattaccactgctttgcctgcggtgaacatggagatgcaatcgatctgg |
| | nitrilase | tgaagggagttcttcatatcgatttcaaaggtgcattagagtggctgtcaccaaactctactaccacccctgtaaatagggcgagaaaacagaaggctatgcagcctgagca |
| | (UG10) | gccagaaggctcagggcttgcgcaagcttataagttatacctgttaagcaatgacaagcaacgactagctaactgggtgactgatcgcaagcttgatatttttttgatggaagat |
| | | gcaggattcatatacgcacacaaaaactcactatctaaacaggtttcctcaagaaaagattttggaacgaagcgtgaattagcagcaacattggaagaagcgaacctaatac |
| | | gcaaaatccttccaagctcggggttccaaaactactatttaaatctacagtcaatccacgacaacaactatatagactttttttcaggggatcgaatcgtattcccgataagagac |
| | | gatcagaaaaaactactaggccttgccgcccgggcggtagatgagcaaccagcaaaatacctattctcaaaaaactttccaaaatccaaagctatttttagaatagagcaagc |
| | | tacaaccactctacgagcattggctaagcgaggcgaaacagatctacgcttatatatctgcgaaggattttttgacgctctaagattggaaagcttgggatttcctgcagtagca |
| | | gtaatgggaacatcaattagcaaagaacaaattaagattatgaaagggcttagcgacacgctcccttcaaagctagcctctttgacaatctgtatttgttttgatcgcgatgaagc |
| | | gggattaagaggagcatccgaggctgtactaaaattcttaggcgctaatctcgacgtggtatttgtatggcctactactgctcagcttacaagcgcagaccattcaaacacaag |
| | | cataaaagatcctgacgaatatttgagaaatttgtccgcgccgcaggccaagtcacttatcgatgtttccacctatggacctgtagtagcagtactagcaaatcagtttggtgtg |
| | | catgccgacgaactgcttgaaaatctaaagtggaacagtgccagtcgctctcgaaaatacaggtcatttgagaaaactcgtgctgaactcaggaaagttgtagccaaccccc |
| | | atctccaatcaagcgacctttttttaaatggccgaacagatcttgactcggcggctcaaatagaatggattgattttttaagtgtcgacattgcgactgaagccgctccatcggaa |
| | | tgttatcttaccaactcaggcaccagactaaaccacgcccgactgctcgcctatatgggctcacgaagaggagagttgccctgcgaagaatcaaaatgggagcggttagat |
| | | attgcggcaagtgcattcaatgtgttgctcgctgaacgattggctaatgaaatacatggacccatcgacccgttcgaggccgtatgggtgccgaggtccttcggcgcagaag |
| | | agccgagattaaaggtgatgcctcaacctgaggatttaatagcgcatcagtacttactaaatgagctacttacagaacgctgggatgcttccgctctcggtgttacagcattca |
| | | gccagtgcataccagctgtccgctattaccgcgaagaaagaaaaactgttacgacaggaatatctaccccctcagataacacccaacctattatacttgaacagacgctaagt |
| | | ttcgcctatcaaattgatatggaggttattgagggcaggcagccagcttcagatcagggaatgtttcgtccgttcctagactgctggcgagactttatgcagtcccttaaaaatc |
| | | aagccaaatctataaattacgtgcatgttatccgcctcgatgtcagtcgatattacgaccgcatccgcagacacgtcgtaagagacagcattcaaccatttatacaacaagctct |
| | | ggaaactgtcgctgataatgcaccggcgtttgctgaactgatgaaaatacaagcatctgcggatgaagcagcggacaaatccgcaataattgtcgagcaattatgcgacatg |
| | | ctctttggctacccataccttagccctgataacgggagaattaataaatcagatcccttacgcggtattcctcaaggcccagtaatctcagcatggttaggctcagtggctttgttt |
| | | ccagtagatctcgcggcactggaaatgatgaacaaatacaatgtagacggggaaactcatctagggtatgcaaggtatgtagatgacatagttttactagctagcagctccgt |
| | | acttcttgaggaactgagagagctagttgatcaaaaaactcggagcttagacctggcgttggtcgcgaaagctgacgctattccgccaatgtctgctgaggaatttgcagatta |
| | | tgcaaatcaagggcgagctttagaagcatctggtccagcgtgggaaccaccgttggctggcgatggtgaagcggggtgggagttttggtcaggcactcccccctcagata |
| | | gacaatctgccctgcaactgctatcaaattgggagatatacaaaagcccaatagaaataatcttgcaaacagtgaaaacgtccttcctagctatggatttacgttctagcgagct |
| | | tgcaaagggagcaaggctaatatggtacgttgtagcatccgacctcctctcagctgacattgatccaagcgatgcggcagatttagcgtgggaaatttatgatcgctattggaa |
| | | ggaatgtactgaggagtgtgggtggcagttaaacccggatagtttcggatgggaggcaccgaatctgttcgcacttgagggactggaaaagcttatagatcataaaaatagc |
| | | ctccaatcgggtttaactgctttagaaaataccgttcggcacaaacgcatctctttcctagctagaaccgtgcttggggagcggttcaaactgcatgctcttgaaagcagctcta |
| | | cgcttaagcaccagatagataaaagactagatctcctcgaatggaaagcgtcaaaatcgtgcggaatgcccgttcgtagaactaaatcctacgcagagcgatcaatgtatatt |
| | | cgctcctggcaacccttcaactggttccatgccgcagtagaagatttcatgctcgcggatcagtccagcggatccgacccattgagttcatatgtcactcagttccaatctatag |
| | | aaaagagcatcagacctaatcacgccgcttcttatgagttcttccggtatttactgccatccgatggcagcgatagcgatcttgagtttttctcaaaaacagagaatcgatactcc |
| | | ggcttagcaattcagattttggttgcattagtccctcgggaaagcataatacagattctctcaaatagagcgcgcttactttgtcctctagaagctggtaaaaaactattagtcatg |
| | | ccccctcttcctggcgtcaatcagcaacgtatagttgcttgccagatcgatagctcctcagaaaacaaaatcaaaaaaatcagctcgtttgagtgctatgaaatagattcaacta |
| | | aaaccaataccacatctctagacttttttggtgcaaactctgcgggcgtagttgtgcttacacccacatggaacaccgaagcccaacctcaatccgccatacttcgatcaaact |
| | | cagaagtcccgaaaaatcttttgttggaggtatttgagaaaccgtcaaccggtttcccttccgctattcagggattgaagcacgtagcctcactatatagagccattgtggtaata |
| | | atggctgaatacgagaggcaaaatgatggtttagagcttatacccgcttggccataccttgccacagatatgacctctgggaactgctacctaatttgtgagggcgtaacgaa |
| | | aggagaagtaggaaaccgagcatttgtaagagacggtgggcgggccctaagaaccattgagataccgatatacgaagcccagttgtggcgagccggggttgcgctaagc |
| | | gattacataggcctgcacgacgatattgctaaatttagctcctccgaatccgaaatacctttggatgcgacaacgcttgccgccccgtcacagtacgtgctacgaagccaactt |
| | | cgtaaactgaggggtgcctttgctaactcacaaatagggcggcgcgttatgcccccaagttttcttccggcaagtgttgaacgtgcgcttgagttattggagcattttccggaa |
| | | gactcagatagtacaaagatgcagctaatgcatctgcttgccactgaaaccgaaactgcgggaatgcgcgtccgctatgagaaaaatattgaggtcacagagctcacggtat |
| | | ttctacgtgcggtcgccgacagggttctaacgaaactacccttaagcataggtgaggtcattgctgcaccgactacagcagtcagtggcctgaggagagacctgagtgggg |
| | | tcttgacccttgccagaagcatatggtcgatggatgaagaagaaaaactctctccaatttttgcgtggaagatttttcgagctggaattgtaggtattggtatcgctgttgctctac |
| | | gggggattatagcttcactaagaagccacggggggtttgcacgctttgagggatttgattttccagcggaatgggagcttccccctgccacagcagttttatccgaaccggcg |
| | | acaacagataaaaccactgatgaaaatgtaagcctcctcgaccatttccgggtactcgtatcacatctcggacaccgaatgaggttggacgacaacggcgagccacaaatc |
| | | ccagaagaaatcagcacagaaataagaaaatacgctacagcattagcgggcctcactactaaagactcaactgcggtggacgcaagcgactggcctttctttgatatcagc |
| | | gaaaaagtttttgataccctaaatatagaattattagagaacgtcagcaatctaatcaaaaacttagattccgcgcttggtctccaggtaattttggttacgcaacaatcatacggc |
| | | ttcaatgctcaaaccaaacgcttcactgactcaagaggacttgcatgggatataaagccatggatgatctcgcaatacccattgcgtgctcgccacgttgaggagtgttttgatc |
| | | aagaccgtagaatcgtacgtgtatggagcgagatttacgaaaaaaacagtcaacgcctgctttctatatcagtactaggcgagcctttcgcatcaattgcactatgtaaggactt |
| | | ggaatcgccttatgccgagactaaaaatgtagacagcaagcacaacactgtattaggtcctagcgagcagggttctgaaagcgcacccatagatatttcaccgattcttgaaa |
| | | ctgctgagcctgaggccgagactgccttagcagacacacaattaataccaaccccaaaccaaactagcactgaagacagctttgataaaatagatactgagcgtaatacaac |
| | | acacaataaaaaactaccgcttaccgacgcaacactcaacgcccgaaagaattcatttagaaatagccagctaacagcctggagcgataggaagtccaataaaaaccctgc |
| | | ccatgttcgggtagctctatttcagtgggaccaagagctgagctatgcacaccctatggtggaggccaccccacaaaaatggcctttcagttccgtctgtaaaccagcagtttt |
| | | aaaagaacttaaacgcctatataactctccctatcaagcccttttgaatgcaactgaatctgccggtcaacaccacctatggaaaaacgaaaatatttccctacccagctggggt |
| | | gagcttcgtcgtcggcgattattgctcaacgcagtgaacgcatgccagtcatttggcgtggacttattgatacttcctgaatactcagtccgtgcagaaactgttaagtggttaaa |
| | | agaagagtgcttacccggaaagacggtagcggttttagcaggaacatttttagctttcgactccggtccgccccccctaaaacaaagcgcgagcctcaacctcttgtggccc |
| | | gtaccgcgtgatattgccgaatgcctcaaaccgcttgcacccaaaacaaatgaagatgctatgtccttgagtgacaagattgacaagggcattgtattgcaatggggcagatc |
| | | aaagaaataccgatcagtagctctaaatgagttcatccggcctggaactgatcctctcacccccctgttcatgcccggaaaaataatagatgaattgagacgtgcaaattggg |
| | | atctggacgctgatggtgttgttaagttgctagccaacacagagttgccacttgcgaatttcatggagctgatatgctctgagattttcctgttcacgagcccaaccaacattcca |
| | | gagatggcaagagattatgtttcaatgtgtgcaagatttggcttcggcgctgcagaagctcaagtctgggcggatctcaaactactatctaaatggctttcggtctgttccaagc |
| | | ctggtggtgccgactctagacgatcaattttgatcgtacctgccgcgaccactcgtactgctgattattggatagcaggccaagctggcttgcttgccgccggcactacaactg |
| | | tatttatcaatggcgtaggatctgggcttaagggtggcagttgttttattggcagagagagctggaaaacaggggctggttctcacggttacattgagaccattacgccatacc |
| | | atggctggtcaaaaggaatttactataatagcaaacatgacccactgagcgaaattgatcaagcattggtgatcgcagatatcgatcctcataacatgcttgaaggcaaaccta |
| | | gacctcagatgctgccagttcccttacagctagtggcatacctaccaatcgttgaaactgtcgacgaaacaagcttggaccaaactctctgtgacgcagttcaggttgaccata |
| | | acaatattgcaagaattaatcagggtcagcgattgggtggacgacttaaaagtcgaaatgagttctggcaacttatcacgcaaagtataaataatgatgtcgacaacgactttat |
| | | cattaacttcagtaaatactttactgatgggaaagcgattcttgagcgagcaaactctttcttcaacaatggacaccaacagcctttttcatcggtagttaagctagacctgctctg |
| | | ctctccggcactttacgactggctagaggccgatatgacgttgcgggagggtgaggcgttacccaacatctcagtcccttcatggaccaaataa (SEQ ID NO: 390) |
| 32 | TOPRIM- | atggatcggtttgacattggtgaggtacttgcgaagtcgcctttagatgaagtagtacggcgcctcggcatcgagaccgagaggcggggaaaccaactcagtgcaatctgc |
| | RT- | ccatttcaccaagacactcgaccgtcgctgcgtttttttccagcggacagcagatctcccgagcattttcattgttttgcgtgtggcgcacacggccatgcgatcgacttagttaa |
| | nitrilase | gcaagtccaaagtgtagatttcttgccggcggtgcaatggctttcgcagagctttggcatcaaagacatccggcgacagccaaagaatcagccagatcgcaaaggcgccat |
| | (UG10) | cgaaggcgcacaggcattcgcgcttcggatatttgatgagcaccacgatacacaacgattccggacttggtgcgaagagcgagccttcgaggctgatttcctgtaccgcca |
| | | gggggtgcgctgtgtgcctcactcggttctcgtgcaagagttggcgtcgagaagcacaggcgagcgtgttgagctgatcgatggcctgcttgctctcggcctgattaagcgc |
| | | ttgcaacaagcatcccattcggatcagtacaagcttagctttccagatcaattccaattgcagttccaagactacttccacgacgggcgtgttttgatcccgatctatggtggtgc |
| | | cgcaaagcgaccggaactggttggcttcgcgggacgggcactgctggctgtgccgccagaaggagtccccaaatacttgttaagcccagggtttcaaaaagccaaatacc |
| | | tgttcaatgcgccgagtgccttttcgtcagcaacgggggaactgagggacggcgacactgcaacgttatatctcgtggagggcttcctagatgccctacgcctgcaggcgtt |
| | | aggcttgaacgcagtggcgcttatgggcacctcactcagcaatgggcagttagagctgctgaagcacttcgttgatggcctgccacagggcaaggctgagtttgtacttagc |
| | | atcttcctcgacaacgataaagctgggtttgcagggacggatcggttggtgcgacgcctgctgggtttgtccggagttgatctgcgctggattggccttgatggctataccaac |
| | | cgtccgcttggcaaggatccggacacttgtctaaaagtgctttcgagccgagtggaggcaacggactggttgcaggacttcaatcggccggccgaggcagccttgctggta |
| | | tccgaattgggagacattgatgcctccgaactgccgaacgaacgctgggctgaactgaattccagtgctcgggagcgggcggtgtacaagactgcgacgactattcgaca |
| | | ggttcgtggctcgcggcctttacagggcgtgattcagcgactgaaggctacagaagagagttgggctaccgaactttgtgaattgctgggtaccgttgaaggaacacagcg |
| | | gaatcggagttccgtgttgtttctccagggcttggaagagcgcctctctcatgcccgaaatttggcgtatcacggatcgcgccgtggcgagctcccatgcgatgaagaatctt |
| | | ggctgactttggatttgagtgcgcgcctgtttgatcgcattgcccaacaacgattggcagagcgtggctggatccaagccgccccatatgatgcagtccacctgccgcgcaa |
| | | gcttacggctaatactacggtactggatgacccgcgtcgcaaggttatgccacacccggccgatttgcacttgcaacagttgctgctgaatgaactgctgacgcagcggcac |
| | | gacttgctgagtgtcgaaggcaagaccttctcggaatggattcctgctgttcgctggttttctgccacccgcaaagtcgaagtgactgggccgtttgacgacctccccgctgc |
| | | agaaggggaggagaccacattgagttttggctaccaagtagatatggatgtgctggagggcagcaagaccccgtcagaccaaggcatgttcaggccctacgggcagtgtt |
| | | ggcgcgacttcatgagcagtttgagcaggcagtgccacgctatcggcggtcgagtgcatgtgcttcgactggacgcccagcgctactacgactccattcagcgttatgtggt |
| | | acgcgatgcactactggactcgatcaaaggggctttgacgggaaccggggcgggcatcttcggcccactacttggccggagcgaaacagctagcacgcaggaggtcgc |
| | | agaggctctggtcgacaaggtttgtaacttcctctttggccaccaataccggcccccaaatacaagagctgtcggctctagtctggatgcgattgggattccgcagggtccgg |
| | | ttctatctgcatatattggtaccatcgccttgttcccggtggatgctgcggcgcgcaggttcatgcgtcgcaacgtccgaccggggcaggatggtatgaacctgccccgcgtg |
| | | ggctatgcccgttatgtggacgacatcgtgctgttcgcagacagcgaagcgctgctggccgagttacaagaggtcctccagaccgagtcagctaagttgtctatctcactgat |
| | | aaacaagggcgaacgcattagatccggcacgccagagcaggtgatgcaccagctcaatgagggacgcagtctggcagcttcggtgccggcttgggaaccaccattcgtt |
| | | ggcgatggtgagtctggatggggtctcggcggcgatctgccagacgtagaccggcaatgcgctttgaaaatgctgcgacatcccgcactgatggacgagccgaaattgat |
| | | tcaggagcaggtcaggcaagccatgcaggctcctgacctccgtccaaacgatctgggcctgtgcgcccgatggttgtggtggcaggtggccactgaactgtccaacgaat |
| | | ctccgcaaaacgacccaagctcggcttggagtcgctactggcagttgtggcgacatgtttgcgaggggcacgactgggccggggagttcgaacgaaggggctacgcac |
| | | agctatacgctgtggaaggcctggacaaattactcgattccaacccttggatggagaatgaacaaacccatagcgaagtaccgcagaaacgggcaattcgtattgggcttgc |
| | | gaagctggtcatctcggcggggttcttctcggaggtgcaaccttctgagaataacgtgcatgtccagcggcgcgcgcgtcttgtggccggtaaggcgcggcagctttccgg |
| | | cgggctgtcgaccactctactaagtcagccacaagacacgcagccggttacgacgatcgagtggttgtgcatggctgctgaattggtacgtgcggcccctgtcgatattgct |
| | | ggcgctgaaggtacgcccccgattctagcgcccatcaagaatcgggttgctcttggcaccgtggatgctgtggcatcgcaggtctgcgaagtgctacggcttgcggatact |
| | | caggatgggaagcttggtgacgtattacccaacccagtgcaggatgacgtagcgcggctagcacttggtttggtgatagataacgcgacccccaatcagcggctggctgtt |
| | | ctgaccaagttcccgggactgctgagtatccgcagtaacggtgacgagctttccttggttcagcgtttacctatcacggagataacgtcactgtgggccttgggtgagccgca |
| | | aaacggggctcgatatctctaccggttctccttgcccccttcgccccttgcgtctcgagacctggcctgcgttgaacttgcgagcgatggcatgccagaggccaggttggag |
| | | gcattgagcttcgaatctacgtcgctcggcccccaatcgtgccctcaccaattggtaagagagaagagcattgaaagtgtttcatgggcgaagtttgacttggattcatcgccc |
| | | aatttgagtcggactgaactggcggttcgcctgtacgtcgcgctagtggccatgcagcggaaggacacaagcgatgctgatctaatgtacgttccttttgcaccacagctattc |
| | | cgatcaggcgatgccacgcagccaacgctgcacttggttgcagaacctgtgaagcgccatacgctaggtgtgagcgcctggtaccgggattgcgatgggcgggtgcgta |
| | | cggttagtgttccacacgtcggtgctgacctatggcgtgcgggctgggcggtggccgacgcattgggcatggcggtagacatgtcaggagaaaccggtctgcgcgatga |
| | | gcaactgtcggacaagacgccgatctcggttgagcactatctactccgtcagcagttgcgcaagctgcagggtgtttacttgtctgaggcccagacattgcgcaaagatgaa |
| | | cagaccggcctgccgcgcacagtaatgcgggcgctgcagcttctgggcgaattcgatggtcgtgcggaacctgaccagcaagtgcgacagttactggttatggaggcgg |
| | | aaacacgggcgatggccttgcgtctacagcagcaggggggcgagagtttgcacgcgctgttgcatcaggtgtttccagccgtgctgaacaaactgcccttgtgggccatcg |
| | | attgcttggccctgcctaaccagcccgccgaacaccaaccgctgcggccagatttggcactcatgctgtcgttgtgcacggccatggagggttattggggccaggggggg |
| | | gcagcgcatcaccatacaaccactccggctctgcgtgcggcgctagctttggcaacagcgggagcagggttgcgtgggagcgttgccgcgctatggggtctgacacagg |
| | | cgcgtggtgccctgcggatgcccgagcgccttgacctgccagccgcttggccgttgcctgatatggtgcgcacggatccgcagtcggactacaaagccatgcgccaatgg |
| | | ctcatcgaaggcgattggccagcgctgtgccgcaccagcccttggcactggatgctcgcgctgaccggtctgttgggtgccaacttcccacaggcttttgaactgcctcagtt |
| | | gcagcaggtctttaccgcgttggcagcttggcagagccaactaagcgctgaggacggcgcctccgtatggccttatgatgggctgccagtactggatccgcagcagtggg |
| | | cgacatttctcgacgcattgcctctggcgatcaggcaaatcgacgatttgcttggcatgcgggtggccccctgtactgccccacggtatcgccgcaacccccataccggcga |
| | | gttcaccgatgccagcaatcaagattggctgcttggcaagtcgcagttcacaggactaggtgctgttgaccgcattgcacggcgtaccaccggcggacgcattctaaacgtc |
| | | tggacagagacccggagaaaggctgacgatgagctactggcagtgcatacgctggatcgaaagctgggggcctggttggaacgcgccgatcaccccgagacagcgta |
| | | cgacggcacgggcgctcctgtggccatgccctcggagaagcctgctggcgaaatcgtcgagcaggtattggctacctttgtgccggatgtcgctgagtctgcctcagacct |
| | | agcccaaagctctactgacgaactgacggagaagcctactggcaaaatcggtgagcagatattggctccctctgtgccggatgtcgccgagcctgccccagaccttgccc |
| | | aaagcgctgctgacgaaccgacggagaggcctgctggcgaaatcgtcgagcaggtaatggctccctctgtggcggatgtctccgagtctgccccagatcttgcccaaagc |
| | | tctactgaaaaaccgacgatgcagcctgtggctgagatggacggcggagccaatattgagtacagcaaggatgttgatcgcttggcggagcacctggacatttcacagaag |
| | | cagtcccgaaagagtcgtgctgatcacaagaattcgaaggcccatttccgcgttgcattgttccaatggcaggtcgaggacacctatacacatcctctgagcgaagtcggttt |
| | | gcgaggcctgcccattggtgaaggggctaaggccgaactgcgtggaatggtcgctgccaatggtgacctctcggtcgctgacaaggccgccaaacggggtgaggagca |
| | | ccaatggaccaacaacgtgaaggtcatgtcctggcatgagcacagacgccggacattgatacgtcaggcattgaatgcttgcaaggatcttggcgtgcaattgcttgtgttgc |
| | | cggaggtctcggttcggagagacacggttgagtggctcgaaggcgtactgaaagactttgaagggttggcggtactggcgggtacctatcgccacttttccaccagagcgg |
| | | aagaccgcgaccaccttcgcgcaccgctgacgttgctctggcggcccgagaccgaaatggccaaggcgcttgggcttgggaatgagaacacgacattcaagttcgaacg |
| | | cggcaagaagtatcgtgcggtggctgctaatgagttgttccggcccgatttgagtcagctctctccgctctacacagaagtgaagctgatggaggaggtcaagagggaact |
| | | caaccgtcgaggacgaagcatgcttgggccagatcaactgcctgagctggctcatgcactggtgcatttgtcgccacccctgcgctattgtatggaactgatttgctcggagc |
| | | tctttctgctgaccagtccggccaattttgaaccactgaggaaagaggtgaacatgctcttgcagcggttcccttcgtactctgaggatacgaagaaattgattcgggatgacat |
| | | cgaggcggtcggtgagctgctgactgttgcccagagaaaccgggagcggcgttcggtgcttctggtccctgcatttacgagccgcagtaacgactattggcacgcagggc |
| | | aggccagtgtgttggcttccggcacggccactgtgttctgtaacgctgcccacaagaacagtgctggtgggagctgcttcattggcattaattcagtgagtcgctcgtcggag |
| | | accgcagggattgttaactctttgacgccttatcacggctggcaaaagggcatcctgcaggcgaactctgaaggggcgctttcgaagcatgatcaggcgcttgtggtcgtag |
| | | atattgatccagtacatgtggtgagtggtaaaccgaggccacagctgttaccagagcccatgtccttggtggcctatctgccagtgatcgaactgatggacaaggaccaaac |
| | | cgctgatggtgtagtgcgtgcattggaggcggaacttgaggatccaggcatggggggtaaagccagggagctgcttgcggcaacgggcttccatgcgcatgacaagtttt |
| | | acagggcttaccagacgcttctcaatgaaaaagggtctgacatcagcaaagcgcacggcgcaaaggcgttggatgattttgtgaagttcttcgcagacccggatgcgttgc |
| | | gcaagcgtttcttagcttggcaagatgaacgacatcagcagccgagtctcgtgtccggaagcctgcagttggagccggcatggctcgatttcttggttgcggatatgacatgc |
| | | atcgatcagatggccaaagtgagggtgccgccatggaaggagaacttgggaataggtgggccttctctagcgagtgactcgtga (SEQ ID NO: 391) |
| 33 | RT | tctccacttcttcaaacatccgtatttatccataaccgcactgttttataaaagattttttgtttttactgttcgtattagtccataactttccagtagaatccagtactaaatgtgtatagg |
| | (UG7) | attatgtatatgttcctgttcgattttggaattctatacacatgcccctaaatgatatgcagattcgccgtgctaaacctgaagctaaagcctatacacttggggatgggcaagggt |
| | | tgtctttacttgtagagccaaatggaagtaaaagctggcgatttcgttatcgctatgccggtaaacccaaaatgatctcgcttggtgtttacccaacgatcactcttgctgatgctc |
| | | gttcccgtcgtgatgaagctcgaaaacttgtggcagaaggaaagaaccctagtgaggttcgaaaagagcaaaagctggctctgcaaacagagtcagagaacgccttcgaa |
| | | aagatagccagagagtggcatcaacagaagtctaccaaatggtcggcgggatatgcatcagacatcatggaagcgtttaagaacgacatttttccttatgtgggaacaaggc |
| | | cagtgggagagattaaaccgctagaactgcttaatgtgctgcgtaaaatcgaaaagcgcggtgcattagaaaaaatgcgcaaagttcggcagcgatgctcagaagttttccg |
| | | ctatgccattgctactggaagggctgagtttaaccctgctgcggatctttcaagcgccctcaatgtacaccaatcaaatcatttcccgttcttaaaggctaatgagatacctgattt |
| | | tcttcgcgccttaaacggatataccggaagtcggcttgtcctgattgccacgaaattgctcatgattacaggtgttagaaccatcgaattacgtgcggcattatggtcagaatttg |
| | | atttagataacgctatttgggaaattcctgctgaaaggatgaaaatgcgcagatcacaccttgtgcctttgtcgactcaagcgttagatttgctaaatgaactcaagatgatgaca |
| | | gggaagtatagttatgtttttccggggcggaacgatccgaacaagcctatgagtgaggcgagtattaaccaagttatcaagcgtattggttatggtggaaaacttactggtcat |
| | | ggatttcgacattccttatctactatcctccacgaaaaaggatatgattcggcttggatagaaatacagcttgctcatatagataagaataatattagaggtacgtataatcatgct |
| | | caatatattgataaacgccgtgatatgatgcagtggtattcagattatatttttattaaggagaatgtgaatgagtaacgagtttgatagtagtaaactagaaaattgctttgagcttg |
| | | cattggaaaatattataaagcacggcgatacagatattttcccttacccatttgaaagtcggttatttgaagatgataaggagaaagtaaaaactgcattaatgcaaacatttaatg |
| | | actttgaaaataaaaggatcgagattccaccaaacataattaatagcttttcaagtattggttattatggttacaggtgggcgacccaaattgatccattctggaatgctttttttcttg |
| | | ggttagttttaaaaatcgctgatgatattgaaaggaatagatctactaaaacgcaggtttattcatatcgctttaaaccaaaccttgctgatggttctctttttgataaagagatctctt |
| | | ggagaaaatatcaagaagacagtatctctgaatgttctaacgatgaaataaagtatgtacttacatgcgatatagcagatttctatccgcgtatttatcaccaccgtttagaaaatg |
| | | cgttagatagagtcgaccccaataaagattactctgggaaaatcaagaaattactacagacatttagtgaaacaaaatcatatggagtaccagttggatgtcctgcctctagaat |
| | | attagcagaactagctctagattctattgataaattattgtctatgaatagaatcaactataagcgttatgtcgacgactttgttattttttgtaactctagagaggatgctcataagatt |
| | | ttaactttgcttagtaaaaaactgatggaaaatgaagggctaactttacagaaacaaaaaaccaatattgttactaaagaagagttcctttcagtaactaaagctaagttgcatgg |
| | | taatgatgaagatgaagaatctcctatgaaggctaaatttatgagtcttcctataagattcgatccttactcagcaaatgcgatagaggaatatgaagagataaaggaatctttaa |
| | | aagattttgacttgttagctatgctgagtagtgagttacaaaaatcaaaaattaaccaatcttttagcaagcatttgataaaggcattctcagcaacatcagatgaaataataagta |
| | | gtgctttcaaagtaatgtttaataacttgcatgagttatatccaatatttacaactataattcaagtagctaactccaactggcaaaaattaagcacagaaaccaaagatattattctt |
| | | gataaaataactgcactaattaaacaagattcatatattttgagtactgagctcaacttagcctatgtagcccgaatgctctcaaaagaaaattcagaaaaatccaccctaatcctt |
| | | agtgaaatatacaataacaatccagaaagcatcttagtcaagaacatagttacacagtcaatggcaaaaattaattcttacgcatggctttctgatatcaaaaaaaatttctctgca |
| | | atgcatccgttgcagagaagactattgatcgtttccagttacatcttaggtgatgaaggacggcactggagagagcataataagaaaacattcaactttgtagaggtgatttaca |
| | | gggattgggcaagtaaaaggcataccgcaagaaatcttgaggatgcgctatgatatctgaattaacgttttctcgaaaattcacttcattttggaatcaattgcttccaaatgctaa |
| | | taatttcatacgcatcattaacggcagtctcatcgaggacgtttatcctcctctagatgactgcgctaataggtcaaataacgtctttgttaatgagtgcgcatttaatttatataggg |
| | | caatacagaatgattcgttagacagaaatattctttcagcacatgatatcttccataatgctgattttcaggttgtttttgaaaaaacaaaagaatatctacagcggttcgcttacggt |
| | | tctaacttcaagctacccttaagcatggttgagtacaatgccataagggaaatagcaagaaacattttgtctcgatatggaatggaaaaccaaattgaagtgtctccacaattcg |
| | | atggatgcggagtaataaataattcatatggcgatatttattattcaaatgttcttgtggaaataaaatcaggagataggaagtttagtgtttacgatcttagacaggtgctaatatat |
| | | ttcactttaaacttttactcaaaaaacaaaagaaacatcaagagatttgagcttttcaatcctcggatgggtatcacttatagtgataccattgtcaaccttagcaaagagttggcgt |
| | | ttattcaacctgaagaattgtactttgagataatgaattctattacagaagaaaatttcatagtaactgaaatgcaacgctagatatcatgcagaccgctacaatccattgtagtggt |
| | | ctatttctaaacgttccttctgacgaataaagccaaaataccaaatagaattaaagaaaattataatatcagccttagcgcgcaatgctccccccgccacgcccgcccgctttgc |
| | | ggggcggttttaatgcagttgcactgacacgctcaga (SEQ ID NO: 392) |
| 34 | RT | atgtcatataatgaaaatgactgggataaagaacatctactatcgtttccaataaatgtgaaagcggtgattgcacatatgcgtcaggacatgagagacgattggtttcctgatc |
| | (UG9) + | ctctatcctataatgacctatttgaaaaagcggatgatctcagagaagtactaatggagttgctgcttgaaggtaatgggcgctatgaagggaatctacgaaatttatgtaacata |
| | PolA | cccaaaaaagggcttggcataagatattctctagaaactgatttttacgatagatttatttatcaggcaatttgttcatttttaattcctttttttgatccattactttcgcctcgagttttag |
| | | ggcatcgatataacaaaaaaagaactaaggaaaagtacctttttaagtctaggattgaattatggcaaacttttgaaggtgtaacctatactgcaatcactagtagtaaagctttg |
| | | atggctacagatgttcttaattattttgaaaatatatctatcgataaagtcaaagaaagctttgagttactaatcccccaggtgaaagcaaatggcgcggaaaaattaaagatcag |
| | | aaatgcaatcaatacactctgtgaattactttgcaagtgggggttcagtaaatttcacggattaccacaaaatagagatccttcttcattcatagctaatgtcatgcttaattctatcg |
| | | atcagaaaatggttgttttaggttatgattattatcgttatgtggatgatattagaataatttgcccagatataagtagtgctaggcgttcactaattgagttaattggtgcattaagaa |
| | | ctattggaatgaacatcaattcaagtaaaacaaaaatacttacatctgattcagataaggatttggtagcagaattttttccgtcacttgatgatagaagtataactatagataatat |
| | | gtggaagtcacggaatcgaagaattattgccagatctgccaagtatattcatgcaatgattaaggattgtatagagagacaagaaacacaatctagacaatttcgatttgcagtt |
| | | aaccgcttgataaaacttgttgatgcaaatgtttttgacgtacattcttcattaggtgaagaattgcttgatatgattataagtacctttatcgatcacccagcctctacagatcaatac |
| | | tgtagattaatttgtgctttgcagccgttagataaacattttgaaaaaataacagatttcctatgtgatcatgattctgcgatacattcgtggcaaaactatcatatctggttgacttta |
| | | gcctaccataattttaaatcagaccagttaattgagacggcatgtgagcggttgaatttaatttcaaatgatccagaggttgcggctgtatttatatacttgtcttgtattggtgagac |
| | | ggaaaaactcattccggtaatctctcaatttgatgccagttggcctaacaggcatcaacgaagttttcttcttgcaactaaagatttgcctcaagactcattaaaaaaaatagttga |
| | | aaaattgacaattaagcttaggaatacggctagaagggctacgccacactattataataatcgcccgttagcagaacggaagtttcctaagattgttgatctatatgacgaggtt |
| | | accacctatgattgatgctcaacctaaagtatttttatttattaaagattattctgagttaggtgaagataggtattttctattaaatgggaatgtcttctctgaggtttgtgcagagcaa |
| | | atagtatcacaaacagagctgattgtttgccacgattattggttaatcgctccgtcaatttggatgtctattgggtcactcccatctttgattgtagatgtagatgaattccaaattatt |
| | | gtatctggaatgaagaaagaaagattgttaagagactgcaaggatatcacgagaaggtcgaatatatatgaaggtaatgaggacttatgttctaggtattttaaaatatttaacc |
| | | gaactttaccttttgaagaggcggtttttagggactttagccttttactaagggaacattatctttcagttaaaaattatgcatctttaaatgatgagttatatcggtttgaaagtataga |
| | | gattcctgtttcgagatatgttataaattcaatttgcaggggaattaaaataaatcagggccaacttttaatacataaaaaaaaccttgagcatgatttctacactgcattgaaagaa |
| | | tactcagcaaaatataatgtacctcttgaagtacctgatgatcaagatgttatagagtatttagagcctatgggatatgattttacgggtgtagacgtggactatatccttaaatttgt |
| | | ccctatggaaagtaattacgcgaaagatgtattgtcgcttaggaaactatctcgatctagaaacgttcttaattctatacctttaagcacgcgccgtgcttatccgatggttgatact |
| | | tttgggtctattacttctaggatttatttaagagacccatccttgcagaatcttgcaaaaaagcatcgtaacatactaattccggacgatagaaagcgatttgtatatgttgactatg |
| | | atcaatttgaggctggaataatggcagctctttcacaagatgaggagctgttatcattatactcggggaaagatatgtatgtgggtttcgctgagaaacttttcaataatataaata |
| | | tgaggaaggacgcgaagaggttatttctgtcatatgcttatgggatgtcgatgaaatcattgatagatgcagcggtaggatttggtgcgaatagaaaggtggctaaggaaatat |
| | | tcaaaagctttgtctattttgaaaaatggaaagaagggatatggagtgattttgccagaagtggcaagattgggactgctaatggtaattaccttatacgtgatagagaggggc |
| | | cattagatggaaaagagaaacgttcatctgtaagtcaagtgattcaaggaacagcttcattaatatttaaggaagccttgatgtcgctggaagctttgaaagctgtagaattatta |
| | | ttgcctatgcatgatgctgttttggtacaggtgccgttagatttcgaggataaagttatagcagaattgcttgcaaatgttatgtctgaccattttggacaaaagattgtaggtaaag |
| | | cttctatcgacactttctttgaagattaa (SEQ ID NO: 393) |
| 34 | RT | atgtcattatctaatttagagaataaaaaagacgatggtctatttcatttcccaattgatgttgatgctgtgcttcttcatttgaaacaggatatgcgagatgattggtttcctgactgt |
| | (UG9) + | cttcagtatgaagaccttttttataagaaaaacaacattaccgaaaaagtagagggcaagattgtttctggacatggtgtctacgatactgacattcggtttatccacgatatcccc |
| | PolA | aagagtactttggggttaagatattccctcgaaacagacttttacgatagatttatctatcaagcgatttgtagttttttaatgccttattttgacccattaatatcgaatcgagtttttag |
| | | tcatagatacaatgaacatcgaaccaaagaaaagtatatttttaaaaatagaattgacttatggcaaaatttcgaaggcatcaccaagctagggatatgtgatgataactatctttt |
| | | ggtcaccgacttacttaactattttgagcatatttcaattggaaatatccaaaaatcctttatagatttacttcctaaagttaaagcgacaggaaaagtcaaaagccaaattagaag |
| | | cgccatccacactttatgtactttacttgagaagtggtgttttaataatcttcatggattacctcaaaatagggatgcatcatcatttattgcaaatatagtattaaccgccgtcgataa |
| | | agctatggttcaaaaaggctatgattattttcgctacgttgatgatataagaattatatgcaaaaatgaatttcacgcaaaaaaagccttgaatattctcatatttgaacttcgaaag |
| | | cttgggatgaatattaactctaaaaagacaaatatatactcttcgtcatcatcccaaagtgataaagaagaactattccctggtttcgatgaaagaagcattgccattgacaacat |
| | | gtggaaatcaaggagtaaaaacgtaataatcagatctattccagaattaactaatatgttaatcgaactaattgataaaaatgaaactcaaagtcgcaggtttagattctgtattaa |
| | | tagaattataaaactagtctcaactggattatttaaaagtggttcaattctatcaaataaagtagttggcgcattgattaaggcattatatgaacaaccggcctcttctgatcaaatat |
| | | gcaagcttttggttgatttaaaattcacaaaaaaacataaaatcgctttagaggaatttataaccaatgatgagctatgtatttacggatggcaaaaccatcatatttggatattattg |
| | | tctctaaagaatatttccacaaaaaaaataattgaccgtgccaagtgcatttgcaatatacaacccataccatctgaagcatccgcatgcttcatatttttagccatgaatagtgaa |
| | | tttaaatacctagataccttagctgacaaattagacaggacatggtcatttcagctgcaacgccattttctccttgcaattagaagctcaaaaaaaacttcatcaccagagcttata |
| | | aaacatgtactgccagcgatacaaggaaccgtaaggggggttaaaatgaacaaaaaattaaaaaatatttttattcatgcaaacccaaaccctgtctctttttctgaaatctacaa |
| | | tgagttaagtccttatgattgatcaatacaacattcttttatatctaaaagactttcaagctaaagggaaggatcgctattttctatttaaagaaaacttgctatcggaagtacaagca |
| | | gatgaattgtttaatttagactcacatttaatcactcatgattatacaatcatttctgagagtatatttaaaaaatgccataaactccctaataaagttgttgacattgtcgattttaagaa |
| | | atttctattacaagaaaaaatcaccgaaaaaaacaaagattcctttaagataaaagaaatcattaaagacgaattccaagacaaaaatgacttaatagaatactttgagatatttta |
| | | taagaagaagcctttcaatattgatacctatctcttatttgctcataaaatatcagatggatatgagcgtttactcgctgaatcgttggcattaggagagcaggatagatatttcaac |
| | | attgaaattccatgctataacgcattgtgcactcatctggctgctggcataaaaatcaacaacgaaaaattaaaagaatataagaacgagataaattatgattattttaaaaaaata |
| | | aagtcatttagtgaaaccttcaacttcatgtatgaaatgccttctaatgaaagcatcaagcgatatgtcacagagaagggatatagtcttagcgaagagtctttagattatataatt |
| | | gagtttattccaatgcctgatgattttggcaaaaaagttcgtgagttacaaaaaataaatgcaactagaaatacattcttgagcatgcctcactcaaggaacacaatttacccatc |
| | | agttgatgtaaatggctccgtaacttcaaggatatatttaaagtcacccaccattcaaaatatatcaaaaaattacagagacatattcattgctgataaaggatgcgcgttgagtta |
| | | tgttgattatgaccagtttgaagttggcattatggctcactttagcgatgacgagaaattaatcgaaatttattctgatgctgacatatacttaaaattctctgaggatgtatttggaac |
| | | cgctgagaaaaggaaaattgccaagcggttatttttgtcttttacctatggaatgagtaaagaaaacctcattaaggtcgtcgaagaaaatcaaggcaacattagaaaagcaag |
| | | agaattcttttcttcatttaaaaagtttgatgaatggagggcgcgtactgtacaacagttttcagacgaaggtagagtcgggacacttcatgggaatttcttgaagataaaaaacg |
| | | caggagatctctcaaatagagaaaaaagatcgtgcattagtcaagttatacagggcacaggttcattaatttttaaaaaaaccatcatcgaaatatctaaaattaaagatttaaaa |
| | | ataatcatccccatgcatgatgcacttttgattcagcatcctgatgactttaatgctgatataattattaaaatatttgaagatgtcatgagcgatacattaaaaaatgaaaggcttat |
| | | cactaaggcttcattgggaacttttatttaa (SEQ ID NO: 394) |
| 34 | RT | atgaatacattcaaagcagaacaacttctaacatttcctattgatacaaatgcaacattaaagcatctacgacaggacatgaaagatgactggttttatgatgcaattaggtatga |
| | (UG9) + | agatctactctctaataagactgacttgcaacgtgttttagctgaaaatcttaatatcaaccatggtaattataaatcaggtgacaaagctatttatgatgtgccaaaacgtgcattg |
| | PolA | ggtctacgctatactttagaaacagatttttatgaccgctttctatatcaggctatatgtacttttttaatgccttatttcgatcctcttttatctaatcgagtttttagccatcgatataata |
| | | aatatggtaattcaaagtatctttttaagcatcgtattgaattgtggaatacatttgaaaatattagctatgtttcactaattgatgataaaacacttttaataacagaccttctcaattatt |
| | | ttgaacaaataaatattgaatcaattgaaagttcattcattagaatgatagcagaccttaatgtatcaggggcagaaaaaaacacgattagaagtgctattagcactttgaaagttt |
| | | tattagagaaatggtgttataacgataagcatggattgcctcaaaatcgtgatgcttcatcatttattgcgaatgtcgttcttgattctgttgacaaaaaaatggtaaagaaaggata |
| | | tgattattttcgttacgttgatgatattaggattatatgtaatgatgaaatggaagcaaggagagctttgaatgacctgatttttgaattaagaaagttagggttgaatataaattcca |
| | | aaaagacagaaatactcaataaacatagtggaaataaagaggatttttttcctagtaaagatgacactatgactttaattgatactatgtggagatctaaaagtaagaaagttatc |
| | | gcaagatcgattccaattctttttgagtttttaaaaaatcagatcgacgagggaaaaactcaaagtagacctttccgttattgtataaatagatttaagaccttgatatcatctaattta |
| | | tttgaggctaaatcagttttagctagagagattgcagatacattaattggggagctagggaaacagccggtttccacagatcaattttgtaaactcttaatggatttggacttgtca |
| | | aatgagcaaaataaagtcatatctaattatatagtaaatgaaaatgtagcgatatatggttggcaaaattataatttaatactacttatggctcataataaatattttgatgataatttga |
| | | ttgatttttgcaagctgaaaattgaaaagaaaattaaaagcccagaaacaccagcatgttttatttatttggcatcaattggcttgcagaatgaggttgaaaagtttattgattctttt |
| | | gataacacttggccatatcaacatcaacgatactttttaatagcacttcaagacacatcaccaaaaaaattacaaccaatgtttggtaaggtaggatatcgtctaaaagggaccg |
| | | ttaaaagattaaaggaaaataaactatttaaaggcgagtcaatataccttaaggattttaactcgactttaattcaagaaatatatcatgagatatcaccatatgagtaaaggaaaa |
| | | gtggtttttcttgtttatcaaaaagacttttcagaaagtggaaaagaccgatattttatatttgataatgaaagtctttttgaggtaacagtacaagaactcgttagttataaatgtttca |
| | | ttgttacacatgacttttggttgatttcaagctctatatataaaagtgcaaatgtattaccgaataagattattgatgttgtacttttagcaaagattgtatctggagttaaatctgttact |
| | | agtgatactcaaccatgggatatatcaaaaactatcaaaccaatattctcaaaatctgaggactttaattattatatggatgtgtattataggaggaaaagttttgattttgacatatat |
| | | cttctttttgcacataagctctgtgaatattttgaaagtttaagtgaaacttcctatcaacaagaggaaacgagtaggttttatagtttagaattaccagtatataatttaatgactttag |
| | | ctgtttgtagagggataaaaatagataatgaaacttttcgagagcacaaggaaaacttacaattagatttttatcgagaattaaaaaagttttctgagaagcatgatgtattgtatg |
| | | agttaccaaaagaaggtgatattcgggaaaagttaattacattgaattatcatgttgatggcgtgtctatagattttctacttgatttcataccctccatagatggatatacggatgat |
| | | cttcgccgtttgcagaagataaataaaagctatcaaatatttaattcaatatcgagctcctctaatagattgcatcctatagttgaatctcattggacatcaacatctcgaatttattat |
| | | aaatctcctgcaattcaaaatattgctaaaaagtatagggatatttttataccagatgcaggtaagatattgagttacgtcgattatgatcaatttgagatcggagttatggcttatat |
| | | ttcaaaagatcctatgatgattgaaatatatacgagaacagatgcttatagtgattttgctattaaagtttttaacgataaaaataaacgaaaaagtgccaaggtaatatttctttcat |
| | | atgtttatggtatgtcaatggataatataaagaaatctacaataagcatgggagggaactctggcaagcttcaagattactttgaaaaatttgaggtttttgaaagttggaaacaa |
| | | agtgtttggaaagaatttgagagtgaaggtcgaattggtactatcaagtctaactatttaaaaagggcaggtgaaggtaagttaacagaaaaagaaaaaagaatttctgtaaat |
| | | cacgttattcaaggtacagcaacttatatttttaagcttgctctgttagaagtttcaaaagttgatgatatagatatattgatcccaatgcatgatgcggcacttattcagcatactga |
| | | aaaagtaagttctgaaaaatttaaagaaatatttgaaaatgttatgacagaagtattaccaggtattcaaggaaaagcttcattagaagatttctatatttcagaataa (SEQ ID |
| | | NO: 395) |
| 34 | RT | atgagtgaacaattcgtgtccgaggcggcaggaactccgcatctggcagagcaggatgatggtcttaaaaatctgaagttattgattgaatccttcaatacagacaaactgaa |
| | (UG9) + | ctccagcgaacaaaagaaactccaagaactccggtccattctttcaccactactaaaaaaaggtggcgttttagcagacttatttcaagacgggaaagacgttttagcatttcc |
| | PolA | gatcgacgtcgacagtgtcctgcaacatttaaaccaagatatgagggatgactggtttactgacacacttcaacacaaagatcttctctcgaacaaacaatcccttcatgaagtc |
| | | ctacatgaattgttaaatgaaggaaatggacaatatatcggctctttcaggagtgtttacaatataccaaaaaaagggctagggattagatactcgctagaaactgacttttacga |
| | | cagatttatatatcaagcaatctgtaccttcctaatacaattttatgatccactcttatctcatcgagtactaagccacagattcaataaagatagaaaatcagagaaatacatattta |
| | | aaagccggattgatttatggcaaactttcgaaggggtaactagaacggcactcagcaataatcaatcactactagcaaccgatctaatcaattgctatgaaaatattacaattga |
| | | aacaatccgcacagcgtttgagcgatcaattgaacatataaatacttccggtccaaataaagtattaattaggaatgcagtgcaaaccctctgcaaccttttgtcgcgatgggga |
| | | tacagtgaacgtcacggcctgcctcaaaaccgcgacgcatcgtcattcatcgcaaacgttgtcttgaatgatattgaccatgaaatggtgcgattagggtacgattattatcgat |
| | | acgtggacgacatcagggtaatttgtcccaacacgagagtcgcaaagaaagcgttgaccgagcttataaatcagctcagaaaggtcgggatgaatataaattctggaaaaa |
| | | caaaaattttaacccaagactcgactgctaatgaagttgatgagtttttcccaacatctgacgatcgaagcctcacaatcgacaacatgtggagatcaagaagcagaagggtt |
| | | attgcgcgttcagcaaaatatatatttcaaatattgaaagagtgcatcgaagaaaaacaaacacagtccaggcagtttcgattcgcggtaaaccgactaatcaagctgaccgat |
| | | gcaggcatttttgatattcatgcaaccatagcaacagacttaaaagcactcttaattagctcacttgaggaccatgcggcttcgaccgatcagtactgcagacttcttgggattct |
| | | agacctcaacgagcacgagctcaatgatatttacaaccatctcagtgatcatgagcgctcggttcactcttggcaaaattttcatctatggttacttctagcaaatcgcaaatataa |
| | | aagcactaatttaataacgctagcaactgcaagaatagagtccgacatacttcaaccagagatagcggccatctttatttatctaaagtgtgttggtgaagcacaagttttaattg |
| | | ataacatttccaaatttgagtctgcctggccatattaccatcagcgaaattttctattagcctgtagcgattttgatcataatcaactgaaacctttaatttctaagctaggccctaaac |
| | | ttaaatggaccggtagcagagccaagccttattttactaatggtatgcctttggtcgaacgagacaaaatagccatgcttgatctttatgatgagatcacaccatatgactgaatc |
| | | caaaaaagccttactttttatagctgactatacagaccaagggcaagacagaatcttcttatggtcagatggcactttaggtgaagtcaccatatctgatttagtagatcaaaagc |
| | | atgagcttgtctgccatgacttatggttaatcgccccatcgctctatcgggcgacaaacaaactaccatccaacatcacagatattgaagaacttcgaatcctcacttctggaaa |
| | | gaaaaaagaaagagaatcgagagacaagaaagacatatcccaactcctgtcctcgtttgtttccgaagaaactattgcaagatataaagagatttttaaccgtaagataccttta |
| | | gatgaagctgttctgtcttcaattggcgaagccctattaaaatgctcagaagttgtaaaaagcgatgcaaatactgccggtgaatgggagagattcatcacaatcgaacgccc |
| | | cgtaaacgactatctaataagatcaacatcagaaggtatttctatttctgaagaaaaacttagataccataaaaacaaaatagaattcgaattctatatggcattgaagagtttttct |
| | | tccgactacgatatgcctctagaggttccctccgatcaagccgttatcgaatacctagagcctaaaggctttgactttaccggcctagacgtggattacattttaaatttcgtccct |
| | | atgcaatcacattttgcagaggacttaattcgcttaagaaagattcaaaattcacgtagagtattagcagccattcccttgagccaaagtagaatttatccgatagtcgatagcttt |
| | | ggatctatcacctcaagaatctacttcaaagacccgtcgttacaaaatttggcaaaacaccatcgagacattttaattccagataccaacaagcagttgtcctacatagactacg |
| | | accaatttgaagcaggcgtaatggccgcactctccggcgatgagaaactattagagttatataacagtagcgatgtatatgaaattgctgcaaaagaaatatttgacgacaag |
| | | agcaagagaaagcaagccaagaggctatttctttcttatgcctatggcatgaagcgacaacacatccttgctgcagcgcagggctttggtgcagatcgccaaaacgctaaga |
| | | aattctttgagcaattcaagacattcgaagcttggaaagtcttagttcacgaagagtttcaccgtacgggaagaattggcactgcgcttggcaattatatgcaccgtgagcgaa |
| | | aaggagaactaacaagcaaggaaaaaagatctgctatcagccaaattgtgcaagggactgcctcgttaatattcaagaaagcattactatgcttgagttcaatatctgaagtaa |
| | | aactaaaactgccaatgcacgacgctgttttgctggaacatcccgcagactacgacatggatcgggtaatcaatattttttcagaaataatgtctgaacattttcaaaataagatt |
| | | caaggcaaggcgtcattaagccaattccatgaagatctataa (SEQ ID NO: 396) |
| 35 | DUF4297- | gaaatttcgcgacagagatccttaacggtgcgtcgagcttcgacggaattcagaataatgatggtctggtgttcggtgaatcgtgctttgcgcatggcgatctcctatcagaac |
| | STAND | aaaaccagtatgccggatgatctctaaaagtgaatggaccgatatgcagggatgcttacagtgggtcttcgacctttataagcatagtaaagaatagaatatgccaatgtacga |
| | | taatctgtgcactctattacctgcgcaaaaaagtacaccagaattgtttgtctggtttggcaaattgagatcattaggcggcatagcgaatgactttaaatgaaaagcccgattca |
| | | tcaataaagattgttaaaacaaaaaccttgcccccagcagagggcgagcgccgggcaatgcgtggctatatgggccaatatgaaagagccggtgcagccatttatgctgaa |
| | | ttagagcgtgggcaattggagtggataggcgtagcggaccgcagtgcgggtatcgttgatgatttagtacttggatttaatggccttatcgttgggcaccagttcaaaacgtcc |
| | | cgtttccctggtacatttacagtacagacactcttagtagggtctgatggtctgcttaagccattagtttgcgcctggcaaaatctttgtagtgctaacccaacgtctcaggtagaa |
| | | attcgtttagttgtcaacgattatccatcagttaacgacgctcccggaatggaagctccagctcatagcgctgccttccttgatgagtttgaacattatcccaaacgcacgcttga |
| | | ggaatggcgctacagtaactggggccgtttagtcgaaatattatttcaacattcctgcctaggtgacgatgatttcgagagattttttcatgcgttgcgcataattcatggttctgca |
| | | gcagattttatacaattccataaactcagtgcagaacaagcgagactggcgtctgatatagcaaaaatattacctcgactggtctccgataaacgagatagggatcgatggtcc |
| | | tgtgaagaactattatatgaactagggtggaaagatcccaccaaaacacgccacttacatcgttttcccatcggtgctcacgtccaacgcaaccgcgatacggaactacaact |
| | | tctccagacgatacgcaacacaatccagggctatgtggcattgattgggcctccaggttcggggaaatcgaccttgctacagacaaccctagctaccgagtataacactcgg |
| | | gtcgtgcgctatctggctttcataccgggcgctgcgcaaggtgtagggcgcggggaagctgatgatttcttcgaagacatttctgcccagttacgcagcagcgggctgcctg |
| | | gacttcgccttcgagacagcagccaatttgaaaggcgcgaacaattcggtgaactgctcaaacaagctggcgagcgttatcaacgtgatacagtaagaaccatcattattgtt |
| | | gatgggctggatcatatcccccgcgaagaactaccagcccattcgctgttaggggaattgccgctgcctgcagccatccctttgggcgtgacatttatacttggcacccagcg |
| | | actggaactcaggcatctcaaacccgcagtacaggaacaggctgggcatccggatcgtctcgtaacaatgcatccacttgagagagtggcggtcgccaggatggcagac |
| | | gttttaggtcttgattcaaccatttcgcgtgtaaaactttatgaacttagccgcggtcatccgctggcggccaattatctcattaaggcactgttatcggctgatgaacaggacata |
| | | tcatgcatcctcgccggagggatggaatttaatggcgatattgaatcagtttacgcatctgcctggagagaaatcgcaaacgaccctgatgttatgcatgtactgggtttcattg |
| | | cccgtgtcgaagctccgatgccgctgaaattgctggcaacaatcgtagatgctcaggcgatagagcgtaccttaaagaccgtccggcatttactcaaggaaacctcaaagg |
| | | ggtggactgtattccataacagcttccgtctatttgtgctctccaaaccaaagataacactgggcagtatagatgaaacctattcacaacatatttatcgtgaattagctaaactat |
| | | ctcgtcatgcaccagaacattcattacagtcctggctaacactgcgctatctcgcccggtcaggagagcgtgatgaacttctggcactcgcaactccagcatattttcgacacc |
| | | agtttgcacatggacgttcctgttcagagattgatgcggacattcacttggctctgattgctgcgcgttccacgtatgatggtgtaattgccacacggttattactttgccgtgatg |
| | | agatatccagacgaactcaagcactggagtatgccaatgaacttccgcgcgcgatgttaaaagttggcgatattgatgcggcgatctctttcgtccaggactttcccaatgcg |
| | | ggctatgaagttgttgaccttcttttggaacagggtgattttgaccgcgcgaaagaactgtttgagcaccttgagccattatctcaattgcatacccccagattcgagcactatgg |
| | | ggattcgcataatctacaagaattcaaaaaatgggcaaaacgagttgttcacttccgcgacgctgagcaaattaagcaggcaatagactatttgaccgttgaggggtttaaac |
| | | acgccacaagtgtatcaaccgatgaaaatatttcctctattcgcgaacagttaaagtggacagtggtcgaggcaattgttaactggcaatcagacgttaatattcaggatacctg |
| | | caatcagtatggcattcatgtgcaagagataccggttttgatgactcaggctggatttattgctagagacagaggaaataacaccttagcatcggaattatttaagactgccatg |
| | | gcattgtctgattttaatgatgtttctaatggggggcgaagatcgattgcattattttatgccacatcaggctgcaccgatctggcttcaaaattattcgaaaacctttttgcgcctgc |
| | | aatttcgatgggagacaatgaattagaatcaacaaaagcactgacgcttgcagccatggaacatgcgcaactttgcgttttgctcggcaaatccttgcccgacgtagtcacctc |
| | | aacacacgctatcttacgaccgctgcagacacatgcttcagaaacgggacgcttgttggggctgtccataataaatgcctcatgtattccttctggaaatattaaaatggtctgtc |
| | | gcatggtgatgagatatgtaatgcaactcaatagctattctggaaacgatacctatcaggctcaattggcattgacagctacatcaccactgatttgtacattaattaaaatttctg |
| | | cgctgtgtggtaaggttgaatattattcagtaataaatgaaattgataatgcaatgcctgctttaatattaaaaggcaatacactactccggcgtgaaatagcattggcaatgtatc |
| | | aggctgacggtgaccgtgaaagggcggccgccagatttgagcctatggtaaacgagttggtagaaaatacacctagcgagcaactcgagactctgtcagttctggcaaac |
| | | agctttgctgcaattggcgatgttgaccgggcactaaacttacttgcttcgatacatgaccactgtttaggctacgctctggcagcgcgtaaggaccctttatactctgtttggaa |
| | | agacatattgattttggccaatgcggcagacccagaacaccgtgctcaacgaataggtcagttgatacgacaggttgatggtatgaaggaaaccgagggagcatctgccgc |
| | | atatcgtttgacagaagtgttaatcaatgaagcaatgcgtatgaatgcgcacagtggttataccgtggcacagaaactcagcaactgggggctgattccatggccaaatcagg |
| | | taaatgaactggtaattggtatgctagatcgccgtcctgaaatggtgtttctctgtacacaaatttggtgcgggctatgccttccattctacattgaaccctattatcgtgaccctac |
| | | acatgtaggcaattatattgacgttgctgcaaatgcagcggggccttcatcaattgccaaactggtatcaattctattaccggcaatccaggttcatagtcgagctcacgagcga |
| | | ctcacgctaataaatcgcctgagcaaggcggcattaagacacggttataccgataaccaacttgataatgccattactcgatggacttcagaggcccccgaagcccgccgct |
| | | cctacacgccacaaacgtacgacgaagcttcaacccttgacgaacttcaacaggcatttgaatcaaatgattccgaacctgagtatcatgcgccttatcgtttttgtgagcttgc |
| | | agagtccgccgcattagacaaggtggtgaaaatgtatgagtgctggcattgcctgcagtcggatgcacgttgtcgttttttggttgcagagcggctagttaatgcgggggaca |
| | | cgacgttagccagaaaattagttgatgattacgataccagtagtgaccgggagatgtcatggagccaatggttaggaggaaatcgattccgtctcttccacgcgcgtaagcta |
| | | ctcgatggagcagcaattcatcatgaagcatatgaagacttcatcagttcaattgtggctgggaaagagagcaccatgtcgttgctaacagatatggcagacattcttcctgtg |
| | | atctgtgagtcgccagactggcccgccgtctggtctatcctggcagagcagatgtctttcactcgcgaacaccgtattggtgaacttttcgaatttggaaatgaaaatatgaccg |
| | | acgaagagttacttgcggaattgctccatttttcattacgattgcctatcaccgaagctcgacgacacgcagagaaaactgcactaattctggcggtacattcaacaggaggg |
| | | caaatcgtatttgagaacaccataacacgactcctgaacggcacccttgatgaaccattccaggcattgcaaattttgcttttgctaaaacagaaccactttgctgctaaatttggt |
| | | gatttagtctctggccttacgaatcatcgtgatgtagctgttgctgaagctgcgtgcttgttagcacaatattggcagctacctgtatcgattgattttcatccgttgccgttgaccta |
| | | tcgattggcactcgacggagaccctgatcatgaaaatgctctgttagatcctgtgagtggggcaatgcgtattgaagtcgacttaggatggacacaaatgcttcgtcccgttgc |
| | | acggagacttgcagagtttgctgattgtgacgaaatgaacatacgccagcgtgccgcaacgtttattcagcaatggggagggctggcagcctttggccctggagcaacaaa |
| | | aaaaatcgaatctcagttacgcacactctcaatgcaaatcacctatcttaagccccatgcttacattggcatactggcacttcgtcatgtcgctggagagctgagcttggcaggc |
| | | ttgctctcgccaagggataaaccatcgctactggaacaaatggatgcagtacttccgccaactcctcgccctgaaatgcaaatccggccaactggcattaggcgaccgctta |
| | | aagtcaaggatgccccgtggagtgaagctgaagaaatgtggacaaatttggttgacgaggatgttaaaccctggataggtcgtgccgacgaattcgtaatagccgaggtttc |
| | | acaattcaaaatgcatgatacccggcgtgctgaatatcaggtctatcgtattagcgcacctcaaattcatatttctgatgccaaattcatggcatggtatcaaagtttgcccgctgt |
| | | cgtttggctgggaaaaatgatcccacttgacgaagacctcgcaccgacaatagtcaggcgtgtagtaagctccatcgggacaatgtcttcgccgggatatgccattgcattat |
| | | gtcctaatatccagatgcatctgggatggcatgaatgctgcgagatgcctaatatttataccgaccagaactcaacaatcgtagcaagattagtgaactggcgagacgccgg |
| | | gccagtggatattgatgatgattatatatggggggaaggttgctatctgacgctttccaatgcaggcctgatacaagtcaagactctgttcggcgaattcaccgtgcgtaatttc |
| | | gcaagcagggctgttcggcaattgcgacaaggcgaagcgcaaatgataaagacagctcagaatcagttcccgatactgtagcgagacgatttcacaacacggttcgattac |
| | | ctgacttctccaaccatggtctgaagaagtcagggagtgtagatcatgccggcattctgtttctgaatggcgcaggatttcgggtcagggtcaccacaacaggcttgtccttttc |
| | | t (SEQ ID NO: 397) |
| 36 | DUF4297- | ttgtgcgtagcacttctccagtttttgttgaaacagataaagagactaaatcgatcattcgaacccaaaaatggccgatttgatgcagacaacgatttaagccatatctggtagcg |
| | STAND | caatcgtcacctatgacaaaagttacatacttgtaatattctgaattcaatattcttcgtgaaattcattcaatgcttctttgagtagtgttttggcgttatgataatttcctaaatatcata |
| | | aggttatcaggcggtgatgtatgaggcgatttgtctatggcgattaaaaacagcgcaatcatttatgcaggctatgattatcagacactccaaggtgtcaggctactggcggatt |
| | | ggctcaatacaccaactaaatataaccgaatagcatttgaggctgatgcgaaacaagttgatgctccacaaggcattgatgatattgtctgcgaacgtcaggatggtaaaaca |
| | | gatttttggcaagttaagtttacgccagataccgacaaagaagacaatcaactatcatgggaatggttactgaaacgtagtggtcatagtattcgagctcgttctatactgcaaa |
| | | aaatagctgatgctgttgataaagtacctgcggaaagaaggggagatattactcttttgaccaataaaatacctaatcgtgagatagcaacttgcttgcgaaataacaaaatag |
| | | attggaatcaggttccaattgctaagcagcaaagcattattcttcagttaggtacccaggaaagagcaaagcaatttttcgatatattacaaatatgtcatagtgatcaaagttata |
| | | cgcgattaaatagtattgtcccagaactacttcgcaaacataccaacgaggagggggtatatcgcctgattgaacgagctaaacgttgggctatccagcgtaattcaccttcg |
| | | gatggtggatggatatgtcttgaacatattcgtgcagtgatttcaactaatagacctgaacctattccgcagacttttgtcttgccagataactatattgttcctgatgcagattttca |
| | | cgacaaattcattgattcactttttaatcctactaatcgattagttgtcttaactggtgctccaggaaagggtaaaagtacttacatcagccatatttgtcagatattacaaactcgcg |
| | | agtttccttatattcgccatcattattttcttgggttagatgatcgtacgacagatagattaagtcccagaatcgttgctgaagacttgatgtgtcaggtcaaagcattttgctcacaa |
| | | atcgaaatgaaaaattatcatgcagagcacctacataaagtgctggctgaatgtgggcagatatataaagaagaaggtaaacgatttttcatcattattgatggtttggatcatgt |
| | | ctggcgtgataacggcaaagataaatctccactggatgagctattttgccaattgttaccgttgcctgataatgtaacattattggttggtactcaaccagtagatgatgagctatt |
| | | gccatcaagattgttacagaacagtccaagagaagaatggttgcacctaccaaatatgtcaggcgatgctattcgtaaatatctatcgggacaagttgaaagtggccgtatcgt |
| | | attcaattttcatcaaagccagtatgaagaagttttatcacagtgtgctgagttgttgactactaaaactcagggatatcctcttcatgttatctactcatgtgaaaaattacatgttga |
| | | aggtaaagggttatcgcactgggaaatagaaaacctgcctcgctgcgaaggcggaaacattacaaattattataatgaattatggaaaatattaaattacgagcaacgcgatat |
| | | tcttcatctctgttgtgcttttccttttttatggcctgccacatcattttctgagattttttctgagaggactgaaactataccgaatgttaaggctgtaatccatttgctttatgagtccatt |
| | | gctggattaagaccgtttcatgaaagcttgattgtttttacccgtagcacaactgaacatgagaatagaataaaattattattgccagcgctaatttcatggctggagaaaagcgc |
| | | acccaaaccgataaaaaattgttggtactggtcatgtcttgcttacaatggtgatccatatcctttaagaaatggcttaactagagactggatattggaacggttggctgaagggt |
| | | atcgacaggatgagtttattcgattactcactcaggctgaaacttctgctttagccgaagggcattttagtgaggcctatcagcatcgttcacgcaagactcgactacttaatgct |
| | | aggttgcaaatctgggatatgtcgacgttgggcgtttgcagtatgattaatgcttctgaagcattgcttaaacaatatcaatctacccagaatgtcagttcaccaaagatactggc |
| | | aactttggctatcgctttatggtttcgtaatcatttcgatgaagcaaagcgcattacaagattggcgttacaacgctactcaaatgaatcatccgtatataccaataaaaatagcga |
| | | tgagtcgcgtgctgacattcgtttattaatcaaagctgctgttttgactgagtgtttcgatgaaaaatggttggcaaccggttcagtacacaagtggagtgatagtaatattaatct |
| | | gcttatcgaatgtgcggaatataaatcagatataggattactattttcattacatgatgtttttaagcaaactgtcataaaaaataaaatagtaaatgcgattgtcagagttgggattg |
| | | ttgaacaaatagatttagaatactggccacatttttctggtcttgactccgctctgctgcggttatacagtcatttatccactgcacatccatgttcacttataacagagcaaggtga |
| | | aagtgaaatcggtagatatcatgttcatccagaagtatcctacgatgaatggttctatgacagccttttttatcgtcttaatgccagtggagattattgttggctaccggttagcacg |
| | | ggggaaggacaggaggaagtcagcagtcattttctccatttaaatgatttctcagatattattgctgaaagtatggctctaaatattcaacaaagcttcagcgatttttgttcacttat |
| | | tgctttggtatcagatcttaaagatcatcaaatgcaaatccaacagaagcgaatgttttttaaaactgattgggtaagcattgctttaaatttacacttaatcatgcattgcaagccg |
| | | gttaatacggaagaaattgatattattcttaattctgagcatacagccctgtatcggctgcataaaactattcttaactttcatagtagagccttcgaatctgatgcaatagcaaactt |
| | | tctggtatttgaggatgggaggcagaaggaaaaactacaagagacaaatgaatatttggcgaataatcttgagttgtcagagattgcgcttcattatgatctcaatcaatcaattt |
| | | tttttgagcgagtcaagttatgttgggactatggtctgggatacggacatcataaagatatagctctgaatcaggtgctgactgcaataaaaactattgcaactgttgagcctaaa |
| | | tatgcattaacgcagcttgagcgtgtgagtccattggttcataatatttgtgacttcacagatggtgaccatactcaacattccgtaacggaattgtctgcgctatatgctcatctttc |
| | | tccccttactttaagtagtatctatgacagttatgttagcgagggtgagtggtatgatgcggataatgcattaacgcaatacttaaaacatgctgatctatcatcacctttcgttgag |
| | | agtttatgccggacattactagatgatgggcaaattgaaataatacagaatcgtgctaaagacaatgccatattgactacgttttggccggaaatattaccacgaaaaatggatt |
| | | atagtagtagcgcaaaacgttcattaagggggactgaaaaatttgatccagcaaaaatcagccctgctgatgtaactaatttactcaatgttcggtcaagttatgaaaatattcct |
| | | aagtggtatcattattggaaagaccaaggaaaagttacagaagtaattaacgtattgctgccaatcattaataatggcttgccagaatatagtgaatttcgttatatattatctgattt |
| | | atttgaagatacattgcgtttgaaaggtaaaaaatatgcttttcccattttagtgcaggaacatattcagcgaaatggttggggtgaatggggggagtctgatgatcaaacatatg |
| | | ctcggttagataaagttatcagattgtatccggataaaattgatgactttctttacaagacgactcgacttcatcactataaaactaaagaagagaacttggtaattcccgggaata |
| | | agctaacatatttattagtaaatgtaggccgagtggatgaggcgaaaagtctatgtgaagcgatgatttcggaggtagaggcagaaacccagaatcttccgttgtgcaaacct |
| | | caatggcaatgggagggagaattagataacgatatgatcgccgttaaattcatcattcgtcgtcttttttggcctgttcaatgtgtaaaacatcttgtcgctgatcaattgtctcatct |
| | | cttagttaatggtcaatgtgctgaagaaattgaaaatttacttgtagttgagatgggaaatcgtcaactggagtcagaggtggtagatattttaactgttctctggttagctagtttg |
| | | aaaggttataaggttcagaataatatatcttcctttatttatgctcgtagctttctttcagatgcattgctggaggctatcgttccaaatttaccaaacctcagtcgctatcaagtgctg |
| | | tataaacatcctgatgatgatggtaatcactatggctttgaaaaaacacttggcaatgaacttccccatatattttgggatgaagtaaaaaggcttgaggagaaatctggagctc |
| | | cggctaaaatattaatgaaaaaagaatggaatgatatttgttataatcatgttcaacgatgggaaagggttgattatttcttcggttcagagcgtgatggttttactatgagtttttcc |
| | | acaaggaatacacgatttggtatatctgcatacttgagaaccattaaccggcttatcaacgaatttagaatgccaaagcattatgcagaacattattcgatttgtttaatgtcagcc |
| | | aacccattattttattccgtatctaatcaccgacctggttggttacctttatggcaatatggggagattaccacaaaggaaaatgtaaaaacatatgttgaggaatgcctgaatgc |
| | | attcaaaaatgaacaggaaaattcaatattaggagcattgtcattacctgtacgcatcgatgaaaataattggttagatattacggctgttatggggatacaaacagaagaatatg |
| | | cctcttttaagatacaacatgccgactgtggtcatagtgtagatagtttacttcaagcttatagaaatattaaattttcatttgcaaaatgggctgaataccaaaattgtgtaccactat |
| | | tgggaagtacacgcgaattactgagaatagcacggtgggatataatgtacgaatttcgtgggcttttctcattcggttgccaggaacaggttactgcctacccggctaaaaatc |
| | | gtattaacttcgattatcagggtaaaaccatcggctatagtgacttctggcaagcaataccattatcaatttatcctaaggatatacgctcacctgttgctacttacactgcttatgat |
| | | aaggaccttgcctgtaactggaaaaatcatagcgtactgaaaaagcctaatatcatgttatgtgattgtaaggtactaaagagagaaaatagttacagtccttttgaaatatcaga |
| | | tattcgttttcactttgaatctgagccgttatagtaaggattattttgcgataattaatcaacggggagctggtcaaagtgcctgctcccatattgactaatatacaaatgtgtttgtta |
| | | agacctttccaaaggtagggggaattatgaatttccgctcctcgctcatagccgcctgccagatttaaccccaccctaccacagggccccctcaagccaagccgccgccaat |
| | | acaattttcccccacaccaaaacgcctccctccctagagcacgtactcacaacgccga (SEQ ID NO: 398) |
| 37 | ATPase_ | atggctaaagcgcactccacgccgctcaacgatattgcgattatcgctgcgaatttaaaagaccgttataaaaatggcttccctgttctgaaagaaattgtgcaaaacgcagat |
| | GHKL + | gacgcacaagcgtcatcattaatctttggctggagccctggtattgctggggcagatcaccctttattgggcgatcccgcgcttttctttatcaataatgcgccgctgacactcg |
| | Helicase_ | aagatgtagaggggatcctctccattggcattggcactaaaccgggtgatgaaaatgcggtggggaaatttgggctcggtatgaaaagcctgttccatctcggtgaagtatttt |
| | SF2 | tttaccagtcctttgactggcatactgcttcggccaaatcagacgtttttaacccctgggacagttacagatcttcttgggccgaggtgagcgagcaggataaagttcgtattga |
| | | ggatgaagtccgcgcaattacccaaaatgcgtgtgatgattatttcgttgtctgggttccgctgcgttcagagagtatctatcaggcgcgccaggatgatgaaaactttattattg |
| | | tcggcgaagactatcgttatgaggtgcctgattttatttcagacccgggactcggggataagctcgccagcctgttaccgctgatgaaaaccttgcaggacattgagctggtc |
| | | gtgaaaacagggcaggggtatcagcgtcaaatacatatctcgctgcctgaaaaggcaactcgcccacaatttaccaatcttaatggtgctggggaatggcaaggccacatta |
| | | ccgttcagcgtgctggattgccggaccctcagcaaaaattctacgtcgggcatgaggttttgctgaatgctcctgagttttctgccctgaaatcacaacgcgcctggccattca |
| | | gttattcacgagaaggtaagaagactgcggataaagcgctgcctcatgccgctgtggtgatgctggcggagaaagtaccagaaggagaggcaacgctggcggtggaatg |
| | | ggcggtgtttttacctttgggtgagcaggacaccgcgcagcatgcgcagaaacaaacattctctatttctggtcagtactcgtatcaaattattctgcacggttactttttcatcgat |
| | | gccgggcgagtgggtatccaggggctggctacactcaccagcgccacgccgttattcaatgccccagattctccaggccaggaacaactggttcaggaatggaaccgctg |
| | | tcttgctactcagggaacgttgccgctattaccgaaagcgcttgcctctcttatgtcgcttattcacgccagggatgcggaaaaagcggcaatttcggatggtgtgcgtagagc |
| | | tttacgcaacaataatgcctggttccactgggtaacgttgtaccatctgtgggtatgcgaactaacgcgggatggaagtcagtggtgtttagttgatgcgaacactcccgttcgt |
| | | cgattgcctgccacaccttcaggtgaagcgcatcgcccctgggaagtgctgcccgctctggaaagtctgggtgtaacgcaccgatttatcgatgaaacgcagcagaatatct |
| | | acaacgaatttaaaagtaagtggcagttgtcggagattcaggtgttgctgcatagcgtacccgaaatggtgttcactagcttaaagcttacaaattatctcaatcaattgctgaaa |
| | | gaactgccgattcagtcagacagctttgtgcttgacctgattgcattgctcagaaaaacgttatttagcgtgccgctggttgagctctcacgtaaccaggcggcgatcggagaa |
| | | ttgatggcgttcattcgtccgacctggcgttacaggattgccattgaccgtcaggagcaggccctgtgggaaacgcttgggcgtaccgctatggataggttgttggttcctgct |
| | | tttctcgataacagtaaagaacctgccagcgcatctctgaattgggagacggttggcagcctgctgcaagcgatgcagaaacaggcttctgccagcgataactttgaaaaatt |
| | | ggtgcgggattttattggcaagctctcatctcccgatcgtcaggagctataccgtcggtttgataccttgaaggtctttaaggtttcacagccaacggggatatcttacctggag |
| | | acgcgctgtcacttgcttgaactaaaacaaaagcgaaggatattcaaacttggcgggagcgctaattttggtatgggtttaagcgcattgttgcagcaggcattgcttgaaaaa |
| | | gaaatcgtattgatcaccaatgatattaaccagaccttatttggtggttctgaatattcagaagcaaaggagtgtgacagcgaaggggttatccatctgcttgagcttcaccctcg |
| | | tctggattcgccgacaaaacgtatcgatttactcaataaaatggctgcggacggggacaaatttagcgccggagatcggcttgtctatcgctatctgatgcacggtaattcgga |
| | | tgatactggtgaagctgaattgtggaaggcgggtaaagcgcatcccgtatgggcaaaaattctttctgatgccgattcggagcaggtcaagtggactattatttcgccagaaat |
| | | tgagcagaatcttggactgactcccggattcgagaaggcgcttaggcttgatagtgtaacgccggatcatgtgatccaccgcttcaaagaaagccttgaatatctggagtttga |
| | | tgacttatctgcagaagatgcggaagaagttctgatgcacattggccgctctatgggcgaaacaatgtggcggcagatggctcttcatcgtagggaaggcaaagaggggta |
| | | tatatcccttgatgatcgttgtttcttgcgtggggggcgcattgaactgcccactgaattgaatgacaacgtgacgttcatccaacccgccagtcagccagagatgcaggatca |
| | | gcagcgcaaatatctgacaatggtgaacgccgaacatgcggtcatgctggctttatccgggccgaacccggaacgttactgcgactttatcctgcaattgttaatgcaaccga |
| | | cgaatgatttgtcttcagagagagcattcaataacctgcgccgccaaaaatggctattgcaccgcggtgtggcgatggcaccagaaaatattctggatattagcgcggcaga |
| | | ctatccggagatcgcgaagctgacagaagcgacgccgctcatcgctctgcttgaggatattgctctcccagatgaggctaactgtgcgctgagttcattggtcgtgcgaggc |
| | | aaggctgcgttttacaaggcgctcactgtagcaggtacacttccactttatgcaatcggtagcagcttacgtctcactgatacgattattcttcaggccagtgacaggtcgtacg |
| | | cgtttgagagctttgacggttggttgctcttaattgagtgtctcaaaggtgctgagtcgcttgagggtaatgaggctatcaatgcgctgagtttttcgcatccggttacagacaag |
| | | atagttgctagctaccggcatctcgttgacagcatgaatccaacccaaagtggtgaattgcgtaaagcactgttaagcacgctgtgtcatacccattcagatcccgccagcgta |
| | | ctgcgttcaatcccgctcagaacggctgctgatacctgggcgttagccaccaatctctgttatggcgtaacgggagcagaacgtagtgctgtcctacatgacgacgactggg |
| | | cgtatttgtccccttggctgcaggctaatgacttgtcggtagacagtactgagtccgaagggcatctcagtcatgttgagcattctgccaatgtcttaagggaatactttgcgccc |
| | | tgggaacgctgggttccacgtaaggcaattgctgcactgctggctttgctggcggggaatcgtaaggttcataagctatgtgagagctacctggggttgcaaagttatgccct |
| | | gttcgtgaatgaactgtcgcaagacagcaaacccttaactaaccatgacgctcactttgcagagttaacgctcttacagtgcattgagaaatatgcctttgccgtgaaggtttac |
| | | gaagaaaacacgttgcaggttcattctctgttccaggaacgtttgaccgtggcgctggcaactgacctggatacgatctttgtgggtcagcacggctacgctttttataccggtc |
| | | aggcaccgcaaatcttcattcgccgattttccccagaccagtatacgcctcagcaacttttggcgattctgaaacgcagcaccagctggctgcaggaaggtatttatctgcaga |
| | | aggcaaggctagacacgctctggcaatcctttgagcaggccgagcagttggatgtgaatatcgcgcgcgtcactatcctgaacagcattgttgagcgcctgaaaacactgg |
| | | gccttaaaaactctcagcttaacgttttaatgagagcctatgagagtgagcttcactctcttgctgaaagtagtgacggcaagttgctccacagctcgaggctcactgaaattgt |
| | | ctatgacattgcaaatgctatccaggatcgccctgaactgcaggctgaaatattaacggcggtcagaaagcgtatagaggatgctcagtatcagccatcaagcgttccttttga |
| | | gctgttccagaatgccgatgatgcagtagaagagttgttcaagctggatagcgatgcccgtcatgagcgggtacaccagaaatttatggtgaaagagcaaaacggcggatt |
| | | gtcattcttcaactgggggagagaaattaaccgctttcagagcgtgaaaaatgagcaagtcgagaatgtacatgatggctacaaaaacgatctgaaaaaaatgctggcgcttt |
| | | accagtcggataaagagcagggcgttaccggcaagttcggtctcggcttcaaaagctgtctgctggtgtctgatcatccttacctattgtcggggcggctggcgactaaaata |
| | | gcgggtggaattgtgcccgaatcctgtgatgctgaaagttataaacaactaaaccaactcactgaaagtgccgcgacaaatggcctgtcacctactcttgtgtatttgccactg |
| | | cgccagcatatgcaagcggaagtggtgttaaaagattttactctgtatgcaggtttgctaagtctttatgcacgtaacttgtgccagattgtcattgatgagcatgaatggcgctg |
| | | ggagcctgttcagtatgcacgtattcctggtctgtcattgggcaaggttatgctgcctaacggcaagggtgctcagtcgccagtgcgggtggtggtttaccagactgaaatcg |
| | | atgatgagcgctgccatctggttttccaggtcacgcgtaggggcctgagaagttttgatactcatattccgcgattgtggaacttgtcgccattgatgagtgatacccggcagg |
| | | gctttttgattaacgctggatttgaggttgatattggtcgacgccagttggctattgaagctgaccgtaatcggggcattatccagaaagcgggagcaaaagttcattcgctgct |
| | | ggaattactttggtgggaaacggagcataactgggaggagctggttgttgagtgggaactgagccctgaattgacccatactcagttctgggaaagcttctgggacgtgatgt |
| | | ctacaggcattagtaacgatattaacgcgatggaaaacgaaaaattgctacagcagctttacgaaagcgaaaatggcatcatgagcttctatcgctcatatcccgcgctgcct |
| | | aacggatttaaagagcaggctgccggactgataacgtggagcgacagagtgcgtagcgcggatgaactggtttctcgtctggcgagttcactgattcatctccctgcgtttca |
| | | ggcattgcacagtgcacagtgcctggtggcagacacgacgggaagcaaacttaaagtcgaaagtaaactgtcgcttgaatcattaataagctcgtcgttgccggataaaca |
| | | gggtgttgatatccagcatctgtcaccgcgggatgctgaaaagctggcagtcgtatttaacgaagagttcgacaagcgactgggtgaactgacaggctggcaggacaaaat |
| | | tgaggctttcagaaaacagctgataaacctgcatgtgcaaacacaagcaggctctacacgcccgattagccaaattttgctcggtaacactccttgtgccgaaaaaaatgaac |
| | | ggatgatctctgggtttgcacctaccgatgccatcatttcatcatcatattctaagcaggcctgtgaatttattgtttattgcaaacgcagaagtcagggatatgtttttgaggattta |
| | | gtcaaatgggcaaagcgcaaaggcctggcggctgataatcaaaagcggcaggcattttgtcgttttctgattgaaggactggaaggggagaaactggcgggtatgctgatg |
| | | gaagagataccaccggactggttgcttgaacttaagctgcgcccaggcgccttcccggcagactggcactggagcaataatgatattgcctctctcctgcaggggcggttac |
| | | tgactaacattgacagaacaaaggcatgggagcgcgagattcgggagacaccggaagaatacgaaccgttggtgacaccaggtgaagccgtacaaaaaatacacacct |
| | | ggtgggagaggaaccagcaggaagagttggtgaaatacaatgctcggctctaccctgaaggctggtttgactgggaagctttaagaaatgcctctgacgatcagcgttcac |
| | | gcctggcgttattgaaactcctgtatctaggctcatgccagaccattgggcggactcaggaagagcaacacagtgccgcaattgagtattttgaggacaaaggctggtggga |
| | | aacctttatcaaccctgatgcagcgcagcaatggctggatgtgatggacaattatctggaggattctttgtacggagatacctaccgtatctggctgcaaatattgcctctgtatc |
| | | gtttttcaaagcatttagattcctatcgcaaactactggatatgtcggaagcgttccttgaggatattggggatttgctgcgaccggcatccagtttcaatctttcgggaacgggc |
| | | gtgggaactgtagtcccggagttacgtgcaactctgggtactggggtgaacttcatcttccgtgaattggtgcgtaataacgtatttatcgattccagcattcatcgatattgtttct |
| | | ctgcgccggaacgcgtcaggcgtctgttactggcgatggagttcgacgaaatggatgttaagcaatccactgccagtgactcgcttctgctgtggacgtttttccgcgaacat |
| | | ctcggtgaggaagatgcgacctttaatcattgtttcgacataccgctgcgcattttaaccagcgaagggaaacgctcacttcgtattgagatatttggacaggatcccctggatt |
| | | acgtatgaaaatgatctttcagcagggccagcaggtacgacatgaacgctttgggctggggacgattgaactcttgcgggaaaacactgcactcattcgtttcgagtcgagtt |
| | | ttgaagaacgtccactttccgaactggagccggtgcgcagtgctcaggatgctttggcagaaggaaattatgacgatctgcgtgaagttctggcgcgcagtcaggcgcttgc |
| | | gatccgctccatcaatgatagttggggggtgttctctacttcacgtatcaacctgctgccgcatcagttatgggtatgtcaccgcgtgttacggcaatggccggtacaaaagct |
| | | gattgctgatgacgtagggttggggaaaaccgttgaggcggggctaatcctttggccgctgctggctaaaaagcgtgtgcagcgtctgttggttttagcgcctgcatcgttagt |
| | | accgcagtggcaggagcgtttgcggcagatgtttgatattcgtttgtccctctactccgcggaaattgatactgagcgatcagattactggaatacgcatccctgggtggtcgc |
| | | ttcattgccgacactgcgaaaagatattaatggcaggcacgagcgaatgctcaaagcagacgactgggacttgctgatcatcgatgaagcacatcaccttaactcgctagaa |
| | | gattcgggggcgactcagggctatcgatttgtgcagaagcttatcgatcacggaaagttcgcctcacggctttttttcacagctaccccccatcgcgggaaaaattacggcttc |
| | | tttgctctgttgaggcttttacgtccagacttatttgacgtgaataagccatttgaaactcagcagcatcatgttcgggatgttgtgattcgcaataataagcaaaccgtcacgaat |
| | | atggacggtgagcgtttgttcaagaccgtcaacgtgacctcacagacctatcatttttctgaggctgaacagtcattctatgaccggctcacacgatttattctttcagggcaggc |
| | | ctacgcttcgtcgctaagctctgcaaaccagcaggccgtgcaactggtgttaacggcaatacagaaactggcggcaagttcggtagcggcaatttatgccgcaataaatgg |
| | | gcgtatcgccaggctcggggaaaatcagaaaaagctgcaggcgctgaatgatgaaatgaatgccatcatgagtgattctcaggccccggatctcgatgatgcctacattgc |
| | | gcttgaaagcgaatatgttgaaatgtctgcttcggttcaacttatgcaaaatgagctgcccatgcttgaagagctgcaggcgcttgcggggaatgtggaatcggaaacgaaaa |
| | | tccagaccttgcttcatgtgctggaaaacacgtttcttaatcgcaccgtcgtattctttactgaatataaagcgacacaggccctgctaattaatactctgaatgctcgctttggcta |
| | | tggttgcgtcagctttatcaatggcgaaggacgcctggaagggatttacaataaacagggcgtcaaaacgtcatggagtatggatcgctaccatgctgcggagcaatttaaa |
| | | agcgggcaggtacgctttattgtttgtactgaagccggtggtgaaggtattgatttgcaggacaactgttattccatgattcatgttgatctgccgtggaatccgatgcgtcttcac |
| | | cagcgtgtagggcgactcaaccgctatggtcaaaaaaatcaggttgaagttattactttacgcaaccccgatactgtagagtccagaatatgggacttgttaaacagcaaaata |
| | | accacagtcatgcgttctttgggcgacgcgatggaggaaccggaagatctgttgcagcttattcttgggatgagtgataaagtttttttcaattcactttttgctgatggcctgaca |
| | | caaaagccagaaactctaaatacgtggttcgattctagagcagggaccttcggtggtcagtcagccgtcagcgtggttaaaggtcttgtaggccatgcggataagttcgagta |
| | | tcagaacttagatgaggttccgaagcttgatcttatccatatgtatggtttcctcgagaacatgctgaaattgaatggacaccgtctggacaatgataagggtgttcttagctttgt |
| | | cactcccaaagactggatcacacagtttggtatcaagaagaaatataacaatatgacttttgaacgtgttcctacagagaaatcgttagaagtgcttgggatagggcatgtgatt |
| | | attaataatgctattaatcaggctgagaaatttaacgcctctacggcagtagcaaggggtatttcctcagctttactgatttacacattgagagaccagattactggcgatagtaat |
| | | gtacaatcattttcagttgttggagtggtactggaagataatattcaaattttggtcaacgctgagttagtcaataaactggcttttatatatgacaacctacctaaaggttcgacgg |
| | | tgattaagcttgacagtgcattccatgttaattttgagagggatataaagcgtgctgaggccgcattagatctctttattcctgggttgaatttaccctatgagcaagtagtatggca |
| | | acatacagcaacttttttgccacagtaa (SEQ ID NO: 399) |
| 37 | ATPase_ | atggcgggtgcttcaatagacgctattggtgtgattaaccaaatcaaagacaacttaacagaccgatacgaggatggctttcctgtccttaaagagatcattcaaaatgctgac |
| | GHKL + | gatgcgggtgcgaacgaattaactattggttggagtaaaggtttctgcaatgcagaaaatgaactactcaatgcgccagcgctgttttttatcaatgatgcaccactggcagag |
| | Helicase_ | gaacaccgtgatgccattttatcgatagcgcagagctcgaaagctacatctaaggcatcagttggaaagtttggtttgggaatgaaaagtttgtttcatatgggtgaggcattctt |
| | SF2 | ctttatgtccgatcaatggcgaattgagcattgggcgtcagatgttttcaatccatgggataagtatcgtgatgcatggaatgaattcggtgaaaatgacaaatgccagatcgca |
| | | acaaagttaaaagggtttttaagtaccgataagccttggtttgttgtttgggtcccgttgcgtacaaaagcgctagctaaagcacacaataactacattatcatcaacaactttagt |
| | | ggtgatgaaaaactccctagtttctttaatcaggctcacttatcagagaaaacttctgagattttgcctcaactcaagaatctcaaagacatcggctttttctgcgagtctgacaag |
| | | ggtgtgtttgatgaagtgacctccatacagttacatgaagattcgtctcgaagctctttttgcggtgaaccgcgattaaataatggagactcttttgcagtcttctcagggaaaatc |
| | | tattcaaattcgaatgaagagcgttgtgcactggactatgcaggatgcgagcgagtcatctttgatgagcgtttaaatcaattaaaagacgaaaatatggggtggcctaagagt |
| | | tatcagttcgacaagaaagcgaacttgcctgttgaggctctcgacaaagctgaacagcatgcttctgtaacattttcgcgttttaaaacaaaggggcaagcgtacctcaaagcc |
| | | aactgggctgttttccttcccttaagccaaaccaaggaacttgttgctgtgcctatcgagggggagtacgactacaatctctatttacacggctacttctttgttgatgctgggcgt |
| | | aaggggttgcatggccacgacaatcttgggttttctacctccctagagcatgtaaaaaatgatgagaaaaagctgcgtgaggtttggaacatcattctagccagtgaggggac |
| | | attcaacctcgttttaccggctctaaatgagttttgtcagaagttaaggctgccacatcaaataaaaactgttttgaccaaggctttgtacgatctcctcatagaaagatatagaaa |
| | | agaagtatccaagagcgccaattggataatcaatatcgatgacaagggggctgcttggtctttacttgataagaatgcccaatgcttaccgatccctcgtccagagaatagtga |
| | | ttactctcgaatttggtcaacgttgcctggtttgagtaagttactggataaaaagtcactgtatgaagccacgggtaatgaatttttaaccgagcagaatcaacgtgatagttgga |
| | | atattacgctcctggaagaagcgttaggaagtggtgttgtcaacgcattttacagatcaatcaatattgaatatctgcttcagttccttcaactagctaaggagcagtgcacgacg |
| | | gaagattttgataacctgattattccacagttccgagaggtattgtctactcataagcttgctgaactttcattgaacaaggctcttaacacgcaagtttttgagcttgttagcgcac |
| | | ctaaaaccgtcgtactaccaattgataaagatgatcaatctatttgggaacttgtctgcaagatcattcctgcaaagctactgctccctaaatttctgtctactcacaataagccaat |
| | | tcatgacaatgtcactgaagaagagctcttcgcacttttaaccctagtagatagctacatcaaaaaacagggtgaacgtttatcctctgatgaatcgtctgcctgtgagcgtctca |
| | | ttacatttgttattgattgtgtaaatgcaagtgaggtaatccaaaaaagcgatttttatcagaagagtgggcatttaaagcttctaaaagtggaagctcttggttcgcaacagagca |
| | | caaaatatcgctccttaaacgaactcatagtgttaaaagaaaaataccagctgtttcttcgtggaggggagcggaactttggtaaagggttggggaaagagctagttgcagtc |
| | | gtgcctggcttggagctttgttttataagcaaggattttgaaattggtggcctatatgaagggcttaccgcttgttctgaagccgcgtgcctacgactgctttccacgtacccaaat |
| | | cttggttcaaattcggcaagactagcgctcactaaagtattctctgccgagctctctacagatgaggagaaaagaggtttccggtatttgattcacggcagcaaagaagacga |
| | | cttgagacaaacgctttggaagccaaacagggcaactaacccagtatggatgaaaatttggcgtatgtgtcagccagaagatttccctggatggtgtgagttagatgaagagt |
| | | tttctaatgctttgacaaaccagtacgaacattttattggcgttaaagagcagttctataaagacattatctctgaatacagaacaatactgcctgaatgcaattttgataactttgat |
| | | gactgggaagtggagcaactgctcgcagatattggtagtcaaggagatgaaaggctatggaaagcgttgcctgtccataggacagctcataacactagagtcgcgattacg |
| | | accaaatgcctgatggaaggaagtgcaacagttccaagtgaatgggatgttcaccttattcaacattcagccattgctgaagtcgccgcttgccagcataaatgggtgaatcat |
| | | ggtctacctaaagagctgatcgagattgcgcttacccaatcaagtccagctcagtattccgcatttattttggaccagctctgcgctattcgtattgcgaatgaaggaattgagca |
| | | tgagttggaaggcaagataaataataccaagtggctgcgattagcgtcaggaaccgaggtttcaccggaagctattttatctttctctgccaatgagctgcctgagtctgcaaa |
| | | gttctgcgagttaaaagagtcaaacatttacatgttctctcaactcgatggaaacatgtttgagcacgatcaagcacgtggtttcttgagagagtgggtcgcaaaaagtaacag |
| | | ctcagtttgctcgtgcattttggcagaagccgcgcaacatcaaagttatgtagttggtaatttttccaacatttctgctcaggtgctagaacagatttcatgcatcccgccattgatg |
| | | cagctatctgcaggctggggcttactggttgagctctaccaaagccaatatctttcagtgaatgaaaacaagcaagtgatgctatgtaaggaaacagaaccacaatcattatg |
| | | gtgggcgctggagcgtattgctgatgatgatattcacggtcagtcaaaggaacttcggaaagcatttttagaagcgttgtgtaacaccgagggaggcgttgattatcttcctaa |
| | | actgagatttcgcaatgagaacggaagttatgtatcgggcaacacactggtatcgaatgttgctcaggtagttgctgataacttaatttcgccacaagaatacgcagtcattgag |
| | | agttattgcagtaaatctgctctcacgaatggtaatacgtcaaaaatcattgagttagcgggcgataatgcgccagtacttagtgattacttcgatgactgggaagggatggttc |
| | | cccctgatgccatagcgacatttatagcactgtttgctaaatctggtggcgtcgagaaattggttaacaattatctaagacagtcaacgctggagtcgataaagcaggggtatg |
| | | aggaaaagtggaactccggaaagggacgtagaggcgaattttcacactatccgtatagctcgttatataaaagtgttgattttgaactggcaatttgtgcagaaaatgcggcgt |
| | | acatgacgtcgattttcggcgaaagaattcaagttaaattacaaaaaacaccagattcattgcttgttcaccaagcgaacaagtccaagacgaaaaggatagagcttcgccga |
| | | gttgatacaaagaatgtatcaaaagaccaacttctccgcatgcttgccaaagctgtagaaacgatttttactgatgtgtttggtgcagagtgtattcgatttgaaagtgaatttttga |
| | | agaggtttggtgcttcagaacaggtagatattcagattacccgacagatagtcttggagaatgttgtccccctacttgaaaggcttcaagtgcgagaagaaggactttgtgattt |
| | | acgttcagattacaaacgtgaacagcgtgttttggcgagcagtgatccttctgtactacaagatcgctcacgccttaacagcgtccttacgaagattaaagagactcttgaaaat |
| | | aacgaaaaagtgcaatctttggtactcgaatctgtacgaaaagagatgagtaaacatttccaatactcgcctttcagcgtgccatttgagctgtttcaaaatgccgatgatgcttt |
| | | gtgtgaacttattgaaatgcagggcgactcaaccaatgtactgactcgatttgatgtggtttctggcagtgatgggactcttaacttctaccattgggggagagaggttaactact |
| | | gtaaaagttcatatgtcgcaggcaaaaaccaatttgaccgcgacttagaaaagatggtgagtctcaacgtttcggataagtcagatggaaaaacaggcaagtttggactggg |
| | | ctttaaaagttcattgcttcttaccgacattccacgtttggtgagtggtgatatttgtgcagaaattcatgctggcgtattaccgagtgttcctagcaaaccagtgatgacggaactt |
| | | aatcaaaatgtcgatgagtataaaattggaaatcgtaaaccgacattaatccagttgcctaaatgtgataagaagcgggcagatttgaagttggttttgggacgtttcaaaagta |
| | | acgctggcattctcacggttttttcacgacaaattcgagaaatcaatattgatgagcagcgatttgggtggtcgggacaggctctccataatatccctgaagtacttgtcggtga |
| | | agtgaaactgccaacaaatacttctgaagagtctaacgttatccttcgaagtaatagagtgcttattatcaataccgagtccggtcagttcctttttgctttggattctaacggagtt |
| | | gtttctctttcgaatcgaaaaaacctaagtagcttttgggtgttaaacccgattgacgaagatctgaaattgggtttctgcatcaacgcgccatttgcggttgatattggtcgctctc |
| | | agcttgctgtagataacggagacaatatcgatctttccagttcactcggcaaagcgttatcagctgtgttggtcaaaatgtttgcagcttcttcgaataattggaatgaatttgctg |
| | | aagaggttggcctgggacaaagcagcacatttatcaagttttgggcgtcactttgggatgtaataacagcccattggccagcaaggcttggagagacgaactctaaagctga |
| | | actgattaaacaaatgttcacagtggaagatggtctgcttgcgttttaccagagatgtgcggctcttcctcgaaatcttggtgtaaaggaagattctcttgttcaacttaaaaacgtt |
| | | gatactggagcgaataaacctttgaccaaggcatttaataccttgggaaatcacccgatacttcaacggctatataaagaccaacaactcgtcgggcatgacacctttgagtttt |
| | | tgaagagtatcgattttagaccgaataatggtgcgttaactaagctcgaattgatcgatttgattggacaggactttcctcacaatgaagtaaaccacgacagagcaagtttctat |
| | | ggtcgcctatttggtaaaaactttgaaaagttaatgtcgaattttgaaatgacagtgactgagaaaaaggtgttggaagagcgtttttctgaattgaagtttctcaacaaaaccggt |
| | | gtatacgtgactgcaagcaaactgattgttgaggggagccctgagagagacttgctatccaagtttgcaccagacagcgcgaagttaagtgaaaaatatgaccaagcatcaa |
| | | tggacttggttagcttcattcgtcgtgacgtaagctatgacattcattcatgggctaagcaaataagatctgaagaatctaacaggggaggaaagcaggaagggttgtgtagct |
| | | tccttgttgaaggcggctatttagcatcatcgcttctcagaaaactacagacggatcaccccgcgtttcttacaaagggacgttttgatccgagcgtattaacagaaaaatggcg |
| | | ttggagttcttcaaaggcttcggctttcattagcatttggattgatacagaggaagataaagcaaggcacgtacgacaagcgcaaaaagagtttattccgaatgtgaccaatgg |
| | | tgagcagatcctcgaaaacatcacgaactggtggaatcaatgtcgtaatcaaagcttaattgattatgacaaacagctctatgctcaaccaatgccttggaaggcaatgacag |
| | | aggacttcgagcttgaaacgttagaggttcgtaaaggttggttgaagttgttctatttagggagttgccaaacattaggtttcaataacgatgtagctaatcggaatgttgtttcttg |
| | | gttcgaggacaaggggtggtgggataaactagccgttgccaatggtcctagccctgaagtatggaaagaattaatggaagaatatcttcaaacagcacgcgttgatgagcgt |
| | | tatagagtttggattcaagttcttcctttgtatcgctttgctactaagctcaaggactatgtcgctctcttcatgaacgcttcctttattgataatcttgatgatttgttaaaaccaaatag |
| | | ttcaaacaagttatcaggctctggcatccaagtatctgagttaaaaggaacgctcggtattgggattaatttcattttacgagagttgcaaaggcaccaagttttggagcgtgagt |
| | | attgtgaagatatccaaaagtacgcatttgttttgcctgctcgattacgaaagttactcaaaaaaatgggagcaggtttaagctttgacgcagagccagagaattcagagcgag |
| | | cttacgactatttcgtttcggcattaaatagtgaaacccaccctcttcttaaggactttgacatcccatttagagtcttgttggctgataagcaagcgtttgaacgttgttttaattttgc |
| | | tctagatgagcagtttgaggaagtatatggataacattatacgcgttattcacccaaaattcggtgtcggtaccgtcgaattcgaaaaagctgagacatctcttgtccgatttgaa |
| | | catggttttgaggagtgtttgaaaagtgagcttgaggcggtcgctgatcttaagtccgatcttgtttctggacagagtgtcgctgcctctgaacttgcgttaaaaacattagcgca |
| | | ctcactaaaaagtgttaatgaaaattggagtgttttttctaaatcgaacattaatttacttcctcatcagttatgggtatgccatcgagttctaaggcaatggccaacaaatcaactg |
| | | attgctgatgatgttggtttaggtaaaacgatagaggcgggcttgattttatggccccttatcgagaggaaaagagtcaagcgtcttctgattttgacgccagcacctttggttga |
| | | gcagtggcaccaaagaatgcttgatatgtttgatattcgtttgagtatgtatgcaccagaaaatgatacctcgcgcgtcaattactgggactcaaacaatatggttgtcgcttctct |
| | | acctacgctaaggaacgacaagaatgggcgtttagagcggatgttaaatgctgagccgtgggatatgctcattgttgatgaggcgcaccatctaaattcaacggaagataag |
| | | ggtggaacgttaggctttcgctttatacagacgttgattgaaaatgataagtttgaatcgaagttattttttacagcgacgccgcatcgaggaaaagaacacggattcttctcctta |
| | | ttgcagttgctgagaccggatttgttcaacgttaagcaaatggatgagcgagaaatgcgcccatttgtgaaagatgtgttgattcgaaacaataaacaatttgttacggatatga |
| | | atggtgagaggttatttaaacctctgtctgtgtcctcaagaacttacagttacagtgaacaagagcaacatttctatgacctcttaaccaagtttattgtatcgggtcaagcgtatg |
| | | catcctctttgaattcaagggatcaaagagcggttatgttggttcttaccgcaatgcagaagctcgcttctagttcaattgcagctatcgagagagctctaaaaggacggataga |
| | | gaaacataaactaggtaagcaacgtcttcaggatattgaagttcaacaggctgctttattagaaaagcgtgaggagtcagaatcgcagtctgaaagcgagatatacagtgatg |
| | | aattagcgcaattagaactggaatttattgaaacgacaacgcgggttcaattgatggatgatgagctccctagaattatggagttgttgtctgcttgtcagaaagttggctctgaa |
| | | acaagaattttaacaatattagatatcctagaaacggagttcaaagatagaactgtcgtcttttttactgagtataaagctacgcaagcgctattaatgggtgctttgaataaaaag |
| | | tatggtgaaggctgcgttacttttattaatggtgaaaatcgtcttctgaatgtagagaatggctcaggagtatgtgttgattatgtcaccgatagatacaatgccgcgaagcgtttt |
| | | aatgaaggcaaagtacgatttataatttctacagaggctggtggtgaagggattgatttacaacaaaattgtttttcaatgattcatgtcgacttgccttggaacccgatgcgactt |
| | | catcaacgtgtggggaggttgaatcgatatgggcaagtcaaaaacgtagaagtaatcactcttcgaaatcctgataccgtcgagtcaagaatctgggatttgctgaatacgaa |
| | | gatcgatttaatcatgcgttcggttggcggtgcgatggatgagccagaaaacctaatggagttgatattaggtatggcggatagcacattgtttaatgagttgtttacagaagca |
| | | gccaatcgtaaaaactctgaatctctctctgcttggtttgaccataaaacaaaaacattcggtggcgagtctgtagtgcaaaaagtgaaagacttgattggtagagcagaaaaa |
| | | tttgactatcaagatcttgaggctgtaccgcgtttagatcttggagatttaaaaccgttttttactcagatgctttcatttaatcaaagacgttgtaagtatgatgaaaatggtggtttat |
| | | cgtttttgacacctcacgcatggttggggcaatttggaaccagacgctcgtatgagaaattgcattttgaccgcaaagctaaacagcttgattcagaagctgacatcataggctt |
| | | tgggcatcccatgttttcaaaagcggttaatcaaggagagcaaatccctggaagttacgcgtttcttaacggtatagagaaagatcttgtagtgtttaaggttcaagatcaggtta |
| | | cgggaaccgatgcatcagtaaaagtgagtattgttggactggtgctcgatgataatggcgattgtgaattggtcaaggacgaagaccttatcgggtatttaaacgagtatctta |
| | | aaatttccaatgatgttgactctaaacgtacaccagaggatttagtgtctgttattcaaactgctaatgattatctaatggagaatgtgtcatcaattggcttaccatttaggctgcct |
| | | aattctgaaccattaacggtattctacaaagcaagtaactaa (SEQ ID NO: 400) |
| 38 | ATPase_ | gtcatagtcccttacggagataattcattgaaattaatatcttatacagcacatgtaaatagccgtggtgtatttttatccaatgaatcgttacaaaaataagatgcatgcccaccct |
| | GHKL- | gttctgtgtgaacgctacgaccagctacggatttataccaaaagtaggaattctatatgtcacgtattaccatcaacgttttatggttaaccgtaccaatagcgcggaagtgggc |
| | DUF3684- | atgagcgaagtagcagatcaacagcaattggaaactcagccagcgggtgatgacctcctgcaaggtgtcaaacgcgttctcaggcatgccgttcaggcgtacggggatgg |
| | DUF3883 | gttaaaggtttatcaaagcctgcaaaatctcaacgaggtgattggcacggagtacggtaatcgggtcatttatgagttgattcaaaatgcgcatgatgcgcatacgtccgaaga |
| | | acgtgggcggatagctgtcagcctggtgcttgaaaacctttcacggggaacgctctacatcgctaatgatgggcgagggtttcgccatcaggatgttgaagcggtcaaaaac |
| | | ctggcgatcagctccaaagagattggcgaaggtattggcaataaggggcttggatttcgcagtatcgaggcgctgacgcaatccgtgaggatctattctcgctcaaatacga |
| | | acggcaaggaccgatttgagggttactgtttccgtttcgcagatactgacgaaatcgcgcataatattcgcgatctcggtgttgatgacgcgatcagcaacgaagttgccaaa |
| | | acgcttccccgctatcttgtgcctgttcctctagatgatcaaccggaggatgtccgcacttttgcccgcaacggtttctccaccgttatcgtggcaccgttagaaactgaagcgg |
| | | cagttacgcttgccagaacgcaggtgaaggagctgaccaatcgcgatgttccactgatgcttttcctcgatcgtattaccgaaatcagtatcgaaattttatccccggatgagaa |
| | | agccgaaaagcgcaccatgcaacggcaggaaaaggcgctgggaagtattcctgacgcgcctgatgtcagtctctacgaagtcgatataggtcagcggaaacgctttttagt |
| | | ggccagaagcaatgtcgataaagcgcgcgtgcagcaagcggtgagcgatagcttattgactgcacctcagctaaagcgttggctgaactggcaagggataccggttgtttc |
| | | tgtcgccgttggcctgaacaaatcaacagtaacttctggaagactctacaactttttgccaatgggcactgaggccgcttcaccgatttgcggctatatcgatgcaccatttttta |
| | | ccgatattgacaggcgtaacacgaacatgagtttgcagctgaaccggctgttaatggaagtggctgcggaaacctgtgccgctgctgctttgtccgtcgtatcccgtgagctg |
| | | gatataggtgcatctgcggtttttgatctgtttgcctggacgggggaacatcgtcgcatgatgcaaacagcactggaacggaaagatacttcgctcagcaaagcccgcctgat |
| | | tccggtgatggctccgccaggaaaacagcaatggtcgagtcttgaagaagtcagtatctggccggaggtgaaatttgccatcctgaagccgaaagacgttgccagatacag |
| | | tggcgcgcagttggtttctagcgaattgaatacgccgcgcatagtgcgtttgagggagataacaaaatttccctatatgtatcagtcattagatccttcggcgcagacactggtg |
| | | aaatgggcagaagcctttgccctttcgctggtggaacggaaattctcccctgccagttggaccaaattctatgatgatttggtcaccttgtttgctgcggtaaaagtgaaactca |
| | | acacacttgagaactgcctgatcctgtatgaccgccagggcaaactccggcccgcaggcgggcataacagtaatgaacacaatggcgtttttgtacgtcggcatgtatccag |
| | | aggcgacaaaaagaaagataagcgtaccgggattccgttgccgccagcgattgtttctcggcgctaccggtttctggatgaaaaaatcgtgcttagtgcggcgacgttcaat |
| | | gcgtttaccgtcgccgacctgataagagagtacgatccgatcaaagccctgtcagggctgaatacggccctgagtaataaggcgacagtcagacagcgccaggatgcact |
| | | attgtgggcatttgaggtctggcgcagcagtagtgtcgttgtcgatgtggagctgaaaaaagccgatctccatattcccgtgcagtcgggttggtgtgcggcaagcaaggcta |
| | | tgttttcatcctcctggacgccaacagggaaggttgtggaaagctatttaaccggcgcgatggggatctcgcctgactgccgtctggcagcgggtttgttattgattgagctgc |
| | | aagactggccgggcgtcgtgcaaaacagcaaaaccgactggattaaattcctccgcgtgcttggcgttgcagatggattacagccggttgaatctaaggtaagagcgcgag |
| | | catatggcgatagttggaatagctttttacgcaatggcgacgagcatgaggggtttgatagcgactggagggcagaagtaaagcgggcacatataagtttctaccatcctca |
| | | gacggtctatacctcggaaggaaaaacatggcgattgcccgggcaacttgagcacgcaacattgccagacgatctgagggagctgttgtgtacgctgattttcgcctttctga |
| | | agtcgcagactacggagttttttacctttgaggtcggtcgttttgagcgacagaattcgcaaacagactcccgtacgctgccaacgccgcttggcacttttttacgcactaaagc |
| | | ttggcttgccagcactagctcactatctgaaggattgcattttagccgtccagatgcgtgctgggcttcgcgggagcggcgcaataaacctccgcgtttcctagaccatttgatt |
| | | gagcacaacgttgatattattgaagagagtcaactagcggagcgcttgttttctgcgaaaattggcctacgtgattggaatcataccgggacggcgttggatcgcattaaaga |
| | | actggtctacattgttccgcagttgaacgctggcgataaggcggatttacagcgggaatatcaacgaagctggcgtgatatcctcgacagcgacgaagctcttcccgacgga |
| | | ttggacctgattgtttttcgccgtgggcagcatgaagtgctgcgcggcaacagcgatctgcctcctgcggtgattgtcaccagtattgcacaaaaaattgaagcacaaatgctt |
| | | gcttctgcaggctacgcaatactcggtattggcctggatgagaccgatacactcgtctcctgcctcggtgatacgggacgattttcaccccgtaagattaatgacggcggagt |
| | | gcaactttacctcgatggtaagccgttttatcccgatgagagcgatccgttgcttatctccttcgacatgaactggttaccggaaatcctggttattggtctggcgttactcgggg |
| | | aaaacttagagcggggcgttcacgccaccaaggttgataagcagctgcgcgcaatcagggtacgccgttgtaagaccctctcttttgccgtgcagggcgatgatgccaccc |
| | | caacggagtcgttcgtcagctattcctggccccatgaaacgatgccgacgctgattattgaagaggggctggtgtttaactggcagaccttagcgaagatttcccgcaacctc |
| | | tcacggctggtggataaccggttacgtttcattgaaaccttacttttgcgcctcgcagttggtcgcgataatggctcgttgagtaaaccggatgacgttaccctggcttgggaga |
| | | tgaattgcgatgttcaaacgatccgtgatcattacgcccgactgcgcacggacatcactcatgtgatagacatgctacttcctgtggtgacgtatctcaacggtattgagcttgct |
| | | caggttctcaagcgggaatatgccttatctaggtcagtatttgatgtgcgtagttggatttcatcacatctatctgatagtgatatacctgctgaaaagctgctggacgtgtgtgaa |
| | | acagcaaccgatcgggttgaactccgtaaaatgctgtcgtttgattttcagcaatttaacctggctctggaagcgttaggggaaacaccgctgtccaatgaggatgctctgcgc |
| | | agattttttacggcctttgtcgggcagaggcgttcacatattatcgatcggttacgccgacactatctggcgacctttgataccggcggagatttgtcacaatacgttcagcataa |
| | | atctttgggcttcatttccttcaactctgaatggattttgacacatgaaaccttggaaaaggagatggtggactcgcaggttgacacgcaacttttgagtgcgttaggaccggac |
| | | aatggtgaagagctgtctgcacttaatacgttattagacgcgaatcgtaaaaatgtgcgcgaatttgccatgcaggctcagccgcgagtttccgcctggtgcagacaaaatga |
| | | tgtcccggtgaatgctcactggcagtacaacgatcctcaggcgttttgccgacagctcgaaaataagggctttcttgatttccggctctttgagccggattcactaccggattac |
| | | tgcctgcgcgccgggctatggccaccaacgatgccgcccagcctagatcaggatgtgctgaatatcgacatgaggaaagtttcccaggaaaaagaacgcgctgagcagg |
| | | caaaacggcaacaggaacttgagcgtcgcagtatctttttttccgggcagtcgcttgatacagccagcccgctatttgccgatcaacttcgggaactggcgagtaccgatagt |
| | | agttggcaggtgcgcagccagcacaagacgcaggccttgatggattttggcgtggtgacaatgcgtcaggcgagcggcggaggttgcggaaaaagaaccgggcgtgc |
| | | gtatcgggagcctcgattgacacctgcacagcagcaagccatggggctggcgagcgagtggctggcttttcagtatctgcgcgatcgctttccggattatacggatgaaact |
| | | tgctgggtatctggtaatcgggcttcgttttgcgggggcgaggaaggagatgattcggccgggtatgatttcatagtgaagacgccgaaagtggaatggcttttcgaagtcaa |
| | | atccaccctcgaagatggtcaggagtttgaactgactgccaatgaacttcgtgtggcaagtgcggcggctaaagacgcaagccgacgttaccgaatcctctacgtcccttat |
| | | gtgctttcgccggatagatggtgcgttatcgaattaccaaacccgatgggcgataaaacacgcaatcacttcagcgttgtggggcatggatctttgcgtttgcgttttcagcgg |
| | | caggagaactgacagcaaccctgctcagggaaacctgagcggggtttttaaatatggcctctatggataggggacactttctgcagtaaatggataataagaaagctaacgtt |
| | | gaagtctgattctgccattttccacgacagctaaatgctggatcttctttttaggatcccaacatacctagcagtaggacgtaagtatgcttgagttcatctcgatatccttgtttctg |
| | | aatgacaggcattactatttcgtgggtgtgaaccgatgaagggggtgatgtcattggaaaataatgaggtagtagcaaggagaagttctgctcttatcatagtgaaaaagcgg |
| | | tttgggaacaaatcggaactgata (SEQ ID NO: 401) |
| 39 | TerY-P + | accttcttcgctaactgatggctaatgaggccgtaataaaacttaccttacctgtaaatacttttactactcattcagatcagaatgaagaggtttattttatttcattgaaaattaataa |
| | helicase | ataaaaatattggcacggtatgtgcttatacagaatgccattttactaacaaggaatttaccgatgtcggaattaaaaaaatttcaggtacaaacagcacgtgcattgccggtgat |
| | + HEPN + | tgtgttggcggataccagtgggagtatgtcaacagatggcaagattgatgcacttaatctggggctcagggaaatgcttgatagttttaaacaagagagccgcctgcgcgctg |
| | ATPase + | aaattcaggtcagcgttattacgtttggtggtcaccaggctgaagttagcttgccattgacgcctgctcaccagttgcaaagtattacctccctggaggcaaatggcatgactcc |
| | DUF2357 | actgggtggcgcactatcgctggcctgcgagattattgaaaatccaacgcgaaaatttcagccgattatcgtgcttatctccgatggctaccctaacgacgactgggaagccc |
| | | cttttgctcgcctgattcacggtgaacttactgccaaggcctcccgttttgccatggctatcggtgcagatgccgatgaatcaatgctcaacgaatttgcaaatgatcctgaggct |
| | | cctctcttccacgcagaaaacgcgcgtgacattcgccgttttttcagagcggtaagcatgagcgtcagcgcacgaagccgttccgcaaccccgaatcagtctacaccgttgc |
| | | agatcccgagtgctgatgatcaggactgggagttctgatgcgcctgtacgcttctggcacctcggtacgtggtcccgcacaccaacaggatgatgaacccaatcaggatgct |
| | | gtagggatttacggtctgcgtggtggctggtgtattgccgttgctgacgggttgggtagccgatcaaaaagtcatttgggttcccgtaaggcagtcaatctgctgcggcagatc |
| | | atgcgcggtgcggagatgctggtcgctgccgaagtgactccagcgttacgtgaagcttggctaaaccactttggtactgactatcacgattacgaaactacctgtttgtgggc |
| | | ctgtgtcgaggcgtcgggccatggcgtgatcggacaggtaggcgatggcctgctgctggtcagaagtgctggggtgttcaacgtaatgagcacaccacgacggggttaca |
| | | gcaatcacactgagactctggcacagcgtgcacatttagatagttgcagtgccagagtggcattaacccaacccggagatggcgtactgatgatgaccgacggtatcgctg |
| | | atgaccttatcccggatcagctggagtcattctttaatgctatctaccaacggatacggcaatgcagcaagcgtcgtacacgtcgctggttaacacaggaacttaacggctggt |
| | | cgactccaaatcatggtgacgacaagagcctcgctggaattttcaggatggactgaccacatgacatcaatagtaaaaacgcaaccaaaacgcgtggtgaaggataccag |
| | | gggatcaagttacgagctgacagaggtaattaaccgtggtggacaaggcattgtttaccggacgacctatccgcaaaccctggtgaaaggttttactaatcaggacccacag |
| | | gaacgccagcgctggcgcaaccatattacatggctgctcagccaggatcttagcgacctcaaacttgcacgtccattaatacttctggcggagcctcgctttggttacgtaatg |
| | | gagctgatggatggcctggttccattggatagcctgttgaacagctttataaacgcaggggaggagtctctggcggattatctgcgtcagggaggactccgtcggcggattc |
| | | gtatcctttgccagctggcacgcacactcaatcagcttcacgcacgcggcatgttgtatggtgatctctcccccagcaatatttttgtttcagacgatccaagacacgcggaga |
| | | cctggcttatcgactgcgataacatcagcctgacagcccatcacaatctgactctgcataccgtggactatggtgctcccgaagtggtcaggggagaatcgttactgtccagc |
| | | ctgaccgatgtatggagcttcgccgtcattgcctggcaactgctgactcataaccatccgtttaaaggggaactggtcagtaatggtcctcctgagatggaagaagctgccat |
| | | gcgcggtgaatacccgtggatcaatgacgcacaggatgacgcgaatcactgcttcgtcaatctgccaccggagctgattgcacatagtgcactgccaactctcttcgctcgc |
| | | tgctttgaacagggaaggtttgaacctcatgagcgtccgggtatggctgaatggcttgaggcgctgagtgctgtggatgagcgtctgtttacctgtgacagctgtgggggaa |
| | | gcacgctcctggcagaggaagcagaaagcgcgaacgatgccgtttgcttttactgtgacagtcccgccgaccgcctcctggtccggtttagtgaatatgtgactgagcaaca |
| | | agacggctcgaatccagacaccaaaaccttgattgccacagggcgaaatgtatggctgcagccaggtcaccgtgttgagttaaagcgcctgttgccaagttttatctatgacc |
| | | actggccatcagatcatctgcagattgattacaccgcccgcgggattgggatccatccgttgcttggcggagagctatacctacaacgcggtgaaactatcaaaccactgcg |
| | | ggggtttcagggactcaaaaacgagctgcgcggaacaggtggggagccttggcagatccatatcggcgatcctggccagtcgcatgtaatctggcagttcacgtggtgac |
| | | aatatatgaaaattaacgaatttccactgatgtccaaagatattctgctgctggaaacggataaaggaaccaccgggttccggccaaagcaagctatcacctttcaggcgtatg |
| | | gtgagaattggctggcggtacagggggatcattgcgtaagtgtccagtgctcccctggtgatcacgaactctttagccgtctggtgatgagggatcaggttcgttggttgctga |
| | | ccagtaaagcggaaaaacagttgcgggttcaatattgcacgcctgttgaagtcacaccaatgcagctcgagttgggaattgatgagcgaattgcggaagaccttttcgcgaa |
| | | aaaacagatcaataacaacgatattgagcttgcctgccgctggtttgaagagacttttattgtccatagcgagtcagaaagtgactggttaacggttggccgttttagcaatcat |
| | | gcagccaaaggtggttttcagctattgggaaacggctggcgtgcggatgttgagcgcaacccggaccacggctttcttatcagacgtattactggtcatttaagccatgatac |
| | | aggcttctcgttgctggttggacacttcgccttccgggatatgtcagttgctgcggtgctgaatagtgcaacccagcaggcaatgctcgatgccgcactgcgagacagtgcc |
| | | agctaccttgagctctggaatctctacaacgataaagagtggcagagcgagttgaaaaaggccgaaacgctgggtgttctgcgctttgttgcgtgcgagggcaccgaagct |
| | | ggccgggaaaatgtctggcatctgactccccgaactcctgaagaatacagagaatttcgccagcgctggcgcgcgctcgatctgcccgcaggcactcaggttgacctggg |
| | | cgctgaaactcccgactgggcagaagaactcagtaccgaagaggatacggtactgaaaacgccgcgcgggaagatcgagttcgctgatgaatatgtggtctttacttcagc |
| | | ctcgaatcgccgagacgtgcgccccgcaaagcctgaaggatggctctacctctcgttggcaggatatcgcacagtcggcaaacgtcgcctggcggcaaaacgtgccattg |
| | | attccggtaaacgcatgccacagttgaagtggctgctggaaggggtcgttgttcctgctgctcggcgtcgcaacatccaggggatgacaccctacgcccgcgaaatctttaa |
| | | gggtggcaaaccaacgggcaaccaggaactggctgtgtttaccgctctgaacacacccgacattgctatcgtaattggcccgcccggaacagggaaaacccaggtgatc |
| | | gctgcgctacagcgacgtctggcggaagaggcccaggaaaagaatattgctgctcaggttttaatcagcagttttcagcatgatgccgtcgataacgcgctggaccgcagt |
| | | gacgttttcggtctgcctgcatcacgtgtgggcgggcgtcgtgcttcagtagaagacgagtcaccactggatccctggttgtctcgccacgccagtcatctgcaggagaaaa |
| | | ttgctgaccagtatcaacgctacccggagttgaaaacaattgccgacctcacttcccggcttgccctgcagcgattggcaaacgacctgcctcaacaacgggcagaggcttt |
| | | ttcgcatatttatcaggacgtcaattccctggcagagaaagggctggtcacggactcccggcttgagatacgtctgcaggactatattaagcatctgaaacaggatggtgttgc |
| | | tgaggtcagtacggtgatgaatgtagcagtattgcgccgcattcgcgcgttacggaccactcagactgctttctcagatgatggtgccgatcgtgcctgggatttgctgcgatg |
| | | gttgaagcggaatgttcctgacatcgacgctgagctgacctcggtattggaaatagctgccgatgccagagaagttcctgtggcactcgtcgagtgccagcaacagctgctg |
| | | gagcgttttctgcccgattatcgacctccggccctcaaaaataagatcgatgatgaaggactggctctactgaatgacctcgacaagcatctttccgacttgatgcatcggcgt |
| | | aagcagggtgtggcatgggtgcttgaacaaatggccgatacgctggagatggaccgccgtgccgcacaggaggtggtggatgaatacgccatggtggtgggagcgacc |
| | | tgccagcaggccgccgggcaacagatggccagcctcaagtcggtttcaggagtcaagagcagtgacattgagttcgataccgtagtcgttgacgaggctgcacgcgcca |
| | | accctcttgacctgtttgtgcctatgtcgatggccacgcggagaattattctggtcggcgacgaccgccagcttccgcatatgctggaaccggatattgaaggccagttacag |
| | | gaggagcatcagcttacggcactgcaactggctgcctttcgttcaagtctttttgagcgcatgaggctaaagctactggacctgcaaaagaaagataatttacagagggttgtg |
| | | atgcttgataagcagttccgcatgcatccactgctgggagatttcatcagccagcagttttatgaaaaagaagggctggggagagtggaaccaggccgtagcgcagagga |
| | | atttgtctttgacgaaggtttcctgagagcgctggggccactggcgtcggcctatcgtgacaaggtctgccagtggatcgacctgcccgcttctgctgggctggcagaaaaat |
| | | caggaaccagccgtatccgcaccattgaagcggagcgtattgctcaagaggtggcacagttactgaaagccggaggagaaaccctctctgttggggtaattactttctatgc |
| | | cgcacaacgagaactgattatggaaaagttatccgaaatcaggctggaaggcgtgccactgatggaaaaacgtaacggaacctatgaaccgcatgaaaactttcgctgggt |
| | | gcgcaagtaccgtgctgacggttcgttcagccaggaagagcggttacgagtaggttcggtggatgccttccagggtaaagagttcgatgttgtactgctatcctgcgtgcgc |
| | | acctggcgtcagccgaggtcctcatctgccgccgatgatgcagctgccagggaacaaatgcttaatgaactgttcggtttcctgcgtctgcctaaccgcatgaacgtcgccat |
| | | gagccgacaacgacagatgctgctttgcttcggcgatgcagcactggccaccgctcccgaagccctggaagccgcgccagcactggcagcatttcataccttatgcggag |
| | | gcgttcatggcactcttcgctgaaacaggtatttatattcaatctgccccacggccgcagggtgaagcgcgcccgatactctggccagtcaggatacatagggtgctctaccc |
| | | ggaaagctatcaggctcagatcaatgtcttccaacgcgcaattctcggattggtacgagcgcgcgtcgtacgtccgaccgaactggcagaactgaccggtctgcaccctaa |
| | | acttattacgcttatcctggcacaaagcgtcagtaatggctggcttgagtccggtgaagataccctcacttcagcgggtcagcggttgctggatgatgaggatgacggtattg |
| | | gcaaacaaaaatcaggctatgtattgcaggatgctgtaagcggaaagttctggccgcgtctggtcagcacattgaagcaaatcgaaccggtcaatcctctggataaatatcc |
| | | gcaatttatactgaccaggaaaacaggagcgacactgcgacctttcctgatgaatgccagccgatcgccactgccgcctctggaacgcaaagaactgaagcgtgcctggc |
| | | gtgactatcgtgacgactatcgtgccagtcagcaactgggcgtcagccgtttgccgccacacattaacctgcacggtctgcagcagctagaggaaccaccgcagtgcgca |
| | | cgaatactggtgtggatcaccactgatcgagagagtggacagctatggagtgccgcggacccatttgctctgcgcagtaacgcatggtggctggacctgccttcaatcgtg |
| | | gaaagtgactcccggttgcaaaagatactggaaccgctggttgtggtgccacgcgccgcagaacaaacctaccagcagtggcttgaggctatcgcgcacgaaactgatttt |
| | | aagatgatgagtcaatacccttgggccgaacgtttaccggatgtgaaacgttatttggtggcgctattggtacatagagggaggatcgagcagggtgataacggtcaaagtg |
| | | agctggatgccgcactgaacgagtgccagaagctgctggaggttgttatgcagtggctgattcgtcgtcatccagccaacgcggaattattacccaagggccgcctggata |
| | | aaattaatacggccaacttgctcaaggatatgaaaataccagcatttaccccatcagttattgatggcctatctggccagataatacgtcaggtgcgctacgcatgtagcaacc |
| | | catccggctcattgaaggcactactttttgcagcggctgtcggtgcgaaccaggatccacagcacccattttggtcactggatgactcagcgttacaactgccaatgctgctgc |
| | | aactggcggatcgtcgcaacaagagtagtcatggacagagtaaatatcttgataagccggtacaggaactcactcagcagatggttgaggaaagtatcagttatgcattgag |
| | | ttttaccgaacgttttaaggaatggatgtaatgtcaaaacgagcacaacagaagtatacctcacctattcccaagcagagaaatggctctgctgcggcatctgccatcaccaca |
| | | cttcagaggtctgcaatgacaaccgagtcgcagattattgccgcagcccatcacacagctcagagtgaaaagcttccaaaagatatcgattttgatgtgacatggctggaacg |
| | | tatcagtcaacgtcttcagcaggaaggagatgatcaatttgtctcctggcttcagacatttactcttttctgccagaaactggcgcaaagggatgaagagacgcaagcagcag |
| | | cacagcgtattcaacagctggagctgacgctggaggagcaaagcgaaaagttagaacaggaccgtgttgaacatgacattcaagctcgggaactggcggaaaagaaag |
| | | ccgggatcgtgagcaaagaacgagagctgaatgaacgtgagctcaacgccaaagcgggcttcagcgagcagaatgcagcatcgctgcgaaacctgacccagaggcag |
| | | cagttactcgaccagcagcatcaggaggatattcaacagctcatcacacaaaagcaggggttaatgcgggaaatatcgcaggccattgtccagttgacccagttacaaatcc |
| | | agcaaagcgacgcggaggcacagcgcagcttgtcactggaccagcgcgaagaagacatcatcaggaaagaggaggatctgaagcgcgccagccgtcgtctggaacg |
| | | agacgagcggtctgtagaggcggagagacaggcgctgaacgaatgtttggctgaagcaatgcaaacagaacgccttgagtttgaaaagaagctggatcagaaagagcgt |
| | | cagttcgacaaagctcaggaacgggtgcaaaacctcagtgaacgcctcatggaatgggaggaacttgatcaggcgctcaatggccaatccgcttcgcaaatgctgaatga |
| | | gctggataagttacgcgatgaaaaccgcgaacttaaaagtcagttcgcgcacactaacctagcagagctggagcgcgagaacaaatctctggccaacagcaaaagcgctc |
| | | ttaaaaatcagctggaaaatctgcttgcagagatggacaagctacaacgcgaggtggatcttcagcgagtggctgcgacccagcttgagacagtggcacgggagaagcg |
| | | gcttcttgagcagcagaaacatctgcttggtcaccagattgatgagattgaagctcgtattggcaagctgaccgatgccagcaaaacccagacgccgttccctgccatgtcac |
| | | aaatggacgagaagaatgggctcaacgcaaaacgtgatcatcgagaggtcggtgacctgaaaaattttgccagtgagcttcagcagcgtattgctcaggcggaagagagc |
| | | gtgcagctattctatccactggaaagtatccagctgctgcttggtggtctggcgatgagccaactgcacctgttccaagggatcagcgggaccggaaaaaccagcctcgcc |
| | | aaggcctttgcaaaagcgatggggggattttgtaccgatatttcggtgcaggctggctggcgtgaccgcgacgatcttctaggccactataatgccttcgagcggcgctatta |
| | | cgagaaagactgccttcaggcactctaccgtgctcaaacaccgtactggcaggacacctgtaatgtcattcttctcgatgagatgaatctttctcgaccggagcagtattttgct |
| | | gagtttctctcggccctggagaagaacagccacgctgatcgaaaaattgcccttaccgaaacagctttactcaatgccccggaacggctcgttgaaggacgccatattctggt |
| | | accaggtaacctgtggtttattggcaccgccaaccatgatgaaaccacaaatgagctggccgacaaaacctacgatcgtgcccatgtgatgacactaccgaagcacgacac |
| | | tcgctttcctgtcagggagatggagaaaaccagctattcgtggcggtcactgcatgaagcctttgctaaagcaaaaacgcaacatgcggaaacggtcaggaacatgctgga |
| | | gcaactgtccggtcatgaatttactcacctgctggaaacagattttggcatcggctggggcaaccgttttgacaagcaggcgatggatttcatcccggtgacgatggcctccg |
| | | gggcagaagctgggcgcgcgctcgatcatctgctggcgacccgtattatgcgctcaggtaaggttaccgggcgctataatattggcttggaatcggtcacacgactcaaag |
| | | aagaacttgaatttttctggattcaggtcggtctgcaaggcgatccggttgaatctatggcattgctggaggcagatatccgccgtctgtcaggtgcgcgctgatgtggcacga |
| | | tcgtttaactggtaggcaacatgcacatcttccgcaacggattgatcacgggcgttactcaatcgaggcttcccctctgacgctaaatggacatacaccgaattttttcggattg |
| | | ctggtcagcgacggcggagcaaattgtcggctggacgatacgctgcataacttcattcagcctccgcccggccatgaagaggaaacccggctgctggaggaagccatca |
| | | ccacgatcggtgccgcagttgatgatgacatcagtgtgctatcgccgctgatgccagcagctattgtcgataatcaaagccttttgctacattcgaacgtgcactgctggagg |
| | | tgatacaaaaaggacatttacagcatatatcacagcggccgcggctggatttacgttatgacgatgaggtggccgacgttgcccgcgtgcgtcgtctggcaaagggtgcact |
| | | ggtacatctggcgtcacactccgaatgctggcagcgtcagacactcggcggcgtggtacccaagcagatactggcacagtttagcgaagatgatttcaatatctacgagaat |
| | | cgggtttatgcgcgattactggataagatcgaacgtcatttgtatcaccggctgcgcactttgagaagcctgcaatctactcttgcccaagcactggacttctatcaatctcagg |
| | | aggtgaattaccgcctgcgcaatgctatttgtcagttgtgggggatgacttacgatgaggatgcgactgatggcgcatctcggcagctcaacgccacattggcgacgctgga |
| | | gcaaattttccgcatcatttccggtctgcgacaaagcggcctctatctgcgggtaagtcgtactgcgcaagtgacaggtggagttcatatgacgaatattttaagtcacgatcct |
| | | cactatggtcatttgcctttactatgggcacagttggctgacggggctcagcccgaaaatttgcctcaacaacgcctcagagtgaaccagagcctggcagctgcgtatagca |
| | | gctatgccgggttggtgttacgccatgcgttgcagccctggttacacggtaagagtgaaggaagctgggctggtcgcactctgcgacttcgccagcaaggcatggaatggc |
| | | tgctgagctgtgattccaatgacagtgccagtgaagagacgctgttgtctctggtgccatttctgaaccaccagcaggtagcggtagacctaccggaaaatcggtatatcgcc |
| | | tggccttgcgtggggcatttacagcaggcattacctgataaagagggctggattcggctttcacctttagatatgtactgtgtagagcgttttggcttactgatagataaaattctt |
| | | agccgggaattattgcgaaactttgcccgtccggttatccgtattccccggtgcgtattaccacttgctacaaaactgtcttcactgacagttgatcaacagttaaatcagataac |
| | | actgcatggggatctgactaaagctgagctggaacaattaacctctcatttaatcaacaacaatgctagcacacaggcagaggaaattacgctgcgataccgggaatggcg |
| | | agcattgcaacagtgccctgtctgcgaccatacaaccgaactggtttatcaatatcccggtggatttaaaaccctctgtaaaaactgcaataccgctcgttatttcagccagcat |
| | | gaaaatgcacacttttttgaacaaaccagaacagtagaaagagaaagtaaaaccttcctggctcaggggcggagagtttttaactttcagttttagcagggtttttacgactcgc |
| | | tgcatttttaaagagttaagaataatgaaacttcagggcatcttttatatatcggtattacgcaaatcagtagtttcggttgcgcgttttgtatacataccggcaagtgtccaatcaca |
| | | gtgaatagccaaaatcgccgggagcacgttcggtcagcctgcggacatggtttttatcacgt (SEQ ID NO: 402) |
| 40 | Kinase- | ggattcaccattatagtgacatgttcaagatgatgatatatctttgaaaagtgttctctttgcgaacggtatagaatttctagcgttacttttcataattacactttttagggttaggcag |
| | helicase | gcacaatctatgcgctgtcttagataactacatccatttttactggactaccaccaacaaaaatttagtggtgcaggagaaaacgtgaagtatcagatagtaggtggtgctggcc |
| | | tgcaccgcagcgaaaccaaaacagttgatatgatggttaagcagttaccagatagttggtttggctatgctggcttagttgttactgatagccaagggtcgatggaaatcgatat |
| | | gctaattattactgctgaccgtctgctattagtcgagcttaaagagtggaatggtaacatcacatttgaaggggggaagtggctgcaaaatggtaagtcacgaggcaaaagtc |
| | | cctatcagatcaagcgtgagcatgcactgcgactaaaagatttgttgcaggaagagttatctcgtaagctgggttactttttgcatgttgaggctcatgtagtgctgtgtggcaca |
| | | gctggtcctgaaaacttgccattaagtgagaggcgctatgttcatacccgtgatgaattcttgactataggtaacccaaaaaattacgaaaagctggtgcaacacactaacttttt |
| | | tcatctttttgaagggggaaagcctcgaccaaattctgatgaggcattacctataattaagtccttctttgaaggaccaaaagtcaggcctttgccactaaaagaaagcggttatc |
| | | ttgcgaacgataagccattctttagtcaccctcacatggtctacaacgaattcagggctacccacaaagacaatagtcaacacagaggtctgctacggcagtggaactttgat |
| | | gccttgggtgtagcaaacgcaatgcaaacattgtgggctgagatagctctgcgtgagactcgagtcggtcgcctagttcgtcatggcagcgcaactatgcaggattatatgtt |
| | | gcgtgctgtaagggaactatccgaggaggatataactgatgatgcccgtgagctgtatgagttacgccgtagttttagccgattagatgagattctagatagcgaagctgacg |
| | | gatggagtaaatctgagcgtattgatcgcgttcgtgcattattagctccattctcggaattacatagcttgggtatcagtcattgtgatattgacccgcacaatctatggtacgcag |
| | | gggatcagaagagcattgtcgttactggctttggcgcagcctcactggagggacataatagcctagagtcattgcgtccgacattgcaaagtgctccatatattttgcccgaag |
| | | atgcttttgaagaagcagttgagccctatcgcctagatgtattcatgttggctgtaattgcttatcgtatttgttttgcaggtgaatcattactgactcctggacagatgcctgaatgg |
| | | agagctccattaactgatccttttagcggtattctaaatagctggtttgagcaagctcttaaccttgagccaagtaaacgctttccacgtgcggacataatgctcaatgagtttaat |
| | | gcagctactaaggaacatagccaagaatttgatgaagctaaccagatttatcaagaattaaagcaaaacaaattctttcgcgaagggatgaacagcgttggtgtgttaattgag |
| | | tttcctccacttcctgaacagttgtctatggtttactctgctcttgctgctattgctacgactggcagcatcagttatcactgtgaacaaggtgggaaagctctgcaggtaaaattgt |
| | | gggatggtgttattttgacccctcaacaacctggtgttaaccgccgtatccacgcttttaagcaacggatcgataagcttacgcatataaatctgccaactcctaaggtgcagtc |
| | | ctatggactattaggacaaggcggcttgtatgtagtgagcgagtatgtggatggcctaccgtggtcacagtttattgctgagaacgtgttagtacaatcccaacgttttacaattg |
| | | cggaaaagttgatcaacaccattcatgcttttcatgaaaagcagttacctcatggagatctttgcccagagaaactgctggtacaagtcggggagcagacagtaattactctga |
| | | ttggattgcttgaattcagtgatgaattaactgcagataatcgctaccagccagagaatcccgaaagtactgatgcttttgggcgagattgctttgcagtatatcgtatggtggag |
| | | gagctatttagtgaagatatgccagtactggtgcaggctgagctagaacgcgcaaaacaaaccgttgacggtatacctatcgcgctcgatcctttgctgcagtcaattcgagc |
| | | accggaacaagctgagattaatcaagttgtggcgtctgagtcacaggataaggtaattcctgtttgctggggcacagatgattggccgcaagaagtgaagcttctagaacaa |
| | | aatgatgggatctattattttcaatgtaactggtcatctaacccacgctttgcgcatgaattgcgttgttacatcactggcctaggagagcggctattgatagacttagatcctgata |
| | | atcgcactattaatagaatagtgtatgaaaaaggattatcgatcgaagaaagtataaaggctggtaaatattcccaggctaaaattaatactcaactttcattacaacgtggctca |
| | | cttaatcagcgtaatacttttattgaactactgtttaacctcgagccagtaattgatgccatcattgagcgagctaatcctaatcaagagatggatgaagatgacttcgatagtagt |
| | | gagtcaagcccaattgagttatggcaggcattatctgatacagaagtagacctacgagatatagtcaacatcgactctactgactttcaggaatcaccgagtggttgcttactct |
| | | acccatatactacggaatccggtgctgacctcagctttgaacttgatgataagatcattgtttatattaaagataagcgtgaatcagtgcaattaggggaattgcagctaagtgag |
| | | actacgccgagtctattggctattcgctttgattttgatgctgctcgtaagcgaattagtagcggcagccagctacaattggaatcgatccgtgacaaatcatcaagagagttgc |
| | | gtcaaagagcccttcaacgggtaattgaaaacaaagcagagatccagcatctgccacagtattttgattaccaccagaaaccctgcatgcagcaaatgcaaccgcggccat |
| | | ccgcggagacattacgcgcactttatgatcagcctggacaacgttttaatgaacagcagctaatggcatttcaacagttggtcgagtttggaccagttggagttctgcagggac |
| | | cacctggaacaggtaaaacaacatttatttcaaaatttattcactatctgtatcaacattgcggtgtgaataacattcttttggtcgggcaatcccatgcctctgttgataatgtagcc |
| | | atcaaggctcgagagctctgccatacgaaaggaatggaactggatacagtacgtattggtaatgaacttatgattgatgagggtatgctaagtgttgcaactaaagctcttcag |
| | | cgacagattcagcataaatttcaccgtgaatatgatctgcgagttagctccctaggaaagcgcctagggatggccccattattagtccaacagttatgtcagttacatcgtacgc |
| | | tgaatcccttgatggtgacatatggccaatatagccgtgagctggataaagtagaacaaataaagagtagtagtattagtcatcaagagcgactggctgaattattagaacaaa |
| | | gcaatcagcttaaactgcgaacacaagaaattattaactcaatattcgatgacagcttgctgaaaactcttgtctatgatgaaaccttgataagacagttggctgagcaagttgc |
| | | catacaatacaattataacaatccagagaaccttgaacgttttatgcagctattggaaatgagccaagagtggatggatgtattacgcggcggcgaggctggatttgatcgattt |
| | | atgttcaaaagtaagcgattggtttgtggaactcttgttggtgttgggaatcgtcgactagaactagctgagtccagctttgattgggtaatagttgatgaggctggccgagcac |
| | | aagctgctgaattgatggtagcgctgcaatcaggcaagcgggtgctgttggtaggggatcataaacaattgccaccattctatcatcaacagcatcttaagttagcctctaaga |
| | | aattagaactcgggaaagggatcttttatgagtctgattttgaacgtgcttttaaagcaacaggcggcgtaacactcgatactcaatatcgaatggtagaaccaattggcgagtt |
| | | agtatcggagtgcttttacgctcaagatatcggtaaactgcattcatcgaggaaagtctcgccagattggtattccaagttaccaatcccttggaacaaaactgttacttggatcg |
| | | atagttcgagccctaatgaagcaggtgcagaagaacataagggtaatggtcgttactataatcaacgagaagtccggctactgctagaggctttgcagtcattgtcgagtgat |
| | | ggctgcattgcacagcttgagcaaactattaccacagaacagccatatcctattggtataatcacaatgtatcgtcagcaaaaagaggaaattgacaatgctatcagtcgggct |
| | | gaatgggctgcatcgttacgtggtttgatcaagatcgataccgttgattcatatcagggccaggaaaacaagataattatcctcagtctggttcgcgataatcccaacaaactac |
| | | aaggtttcctgcgcgacgcgccgcgaataaacgttgctatttcgcgagctcaagaaaggttattgattctgggagcaaggcgtatgtggtcaaagaccaataatgattcagca |
| | | cttggaaacgttcatgaatttattagtaaacaggttgcagtagatgaacccaactaccaaatcctgtgtggtcaaagtctgcttggagataacaactaatgtcagaaccacgtct |
| | | gggtaatctgattaccgttttactacctgcgcgtagttacaagatcaactgcgctttgaccactgaaaaactgatgcctggaattgaacagtttgcatgtcgcttgctgctgattttt |
| | | gatcaactctatcccagcgagttacagaattactttggtctaactgatcgtgagcgagaggtattgcttgatgggttgctggctaacagactgatcaacattaatcctgatgggc |
| | | atattgaggctagctcattcctacgtaagcatgcagctaataatggtgggaagccaagtttagttaaatatcaagaatgtacggaggaagttgcattcgatctactaactctttcg |
| | | atatgtaaaccgcaaccaaatcgtcgttttacttctggactgccagagctattgccgcggcatcagatcgggggagatgctgctgcggtaacagaggcttttagttcccagttt |
| | | cggcaccatcttttgctcagccgcaacagcgagtatgagcgtcaacggactaaattatataagataatgggctgtagttcgcatgagatggtgcagctcccaatagagataga |
| | | ggttagctacggtgtttctgctgggagcattgagccgcagaaatttactcgttcctatgaatatttaggtaacacccggctgccgctttcaaacgagctggaagctcatatcgca |
| | | gattttttgggagaacataaactagatgaattcggtatcgactgtgaagatttctgtaaactagcaaatgataaagtgttgttacaatttgctaatggttataagttcaactattccgg |
| | | ctggatagaggctcgtgaacaacgtaaaactggctacggtacttcattgactaccggcatgttaggggctgtttatttgccgcacaattctaagctgttcattagtatgttgcataa |
| | | tgcattacgtgattatataggtaaaacagctccaaaagcgctgtggtatagcagtaaagtaccactgtggggagctaatggtagtcaactttcgcgttttactcgcgctctaggc |
| | | gatatacttggcaattatgccgatgataagattgctcgcatttcgcttttacactcaagtgcagatgaaggtgaaaaacgtcaagagcgtaagcggcacttaggtcgttttcctac |
| | | cggtattggccttacttcagaggctaaatttgatcgtttggagatcctcttaattcctgatgtgattgctttggtgcaataccacggtcaacctaattctgatagtgcattaaccctgc |
| | | cgattggttatataactgttgagccagagcgtttagaattacttaaaaaactaatgattaagcgaactgaaggggctgttgcaaccattacttggtctgaatcaaaatttgaaaatt |
| | | tagcttcgctattacctgttgagtttctgattaaactgaataagaaaagcggtgaagatgtggatgctgcaataaaaaaaatgcagatctataaccgtgctgaaaccgcacggg |
| | | caattttatcgctacgcaagtagcatttatattgcaacgaataaatttttctaggttgctatgaactagctaaagggcaacaaatagataaacggcgttattcatgtcaaatgagat |
| | | aatgttaaattgatagggatttataccccgccggccattttgaatggtcggagttgttataaacgtta (SEQ ID NO: 403) |
| 41 | Helicase- | ggggcgaaaaggggaatgccggtcattgccggacgagtgcaccttaaaatgtgcggcagggggcgcccgcgggctgatccatttggcagaatggccgtgcatgcgacg |
| | DUF559 + | atcgagcgcgggagacggctgaccctgatggacaaacgcgctttgagcgagcgggacatctgcactaagttcatcacgtcgtggcttgacagatgtttgccttgaccggtc |
| | SMC + | gaatagccccattcggggccgtgtactttgcaaatgggccgaggtgcccgaaaaaccggtctggagccaggacaagaattacagtgcgcgaaccccaccggttactcac |
| | McrB + | agcccgcttattggagttgatcgaaacccatcccgaaggattgcgactcgacgaggttcaggcgcgtacgcgtgttgaagggtgtcgcgcgggagtcgatgatctcgcag |
| | DUF2357 + | cagcgctactcgatctccagcaccaaggtcttgcacatataaacgcagcccggcgctggtttccgaagcgggcggcgagtgtacgaccatcctccgcagtcactggttcgg |
| | ATPase | atgacgtggcgggtgcagggctggtgctgcaggcgctaccggcgcgcatcactggcaacgatatggcggtagcaccagcacctgcattgagtgctaccggcacctcgct |
| | | caagccgacttggggcctgttacgcagcctgctgccgtattacgccgaggcgctagcccgcaatgaacgggcgttgctactcggaacgcctgagcgctacggcgagcag |
| | | ttcctgctcgtggcaccacgcggccgatggtggccagcagcagggttaggctacgggctagaactctcgcgtacgcatctgccggttgcttttctcaccgcgttagcccgac |
| | | gcacgcgcgaaccgattcatgtagcctaccccatcgcgctggtgcggccccgcgacgccgcgcgcagcccctttctgttaccagtggcaactgtggcagcggactggac |
| | | cctcgacgccgagaaactgcgcctgaatctgccggcccaaacgccggcgatcgaatggtcgtgggtgcgcggacagcgccagcgcggacgccagattcgcgagttgct |
| | | cgatgcacttgatgtcaatgctgacgacgaagtctggcgggcaggctccttcgtcgactgggcgaccttcgtcgatcgtctcgctgcaaccacccctaccgaggtgcgcac |
| | | accgctcgatctcgctcagcccaacaatgagttggattgtggccaggcgggcggtatttacggggcgttggggctgttcctgtcgagcgaattgcagttcgcgcgcggggc |
| | | ggtgcgtgatctcaagtccatgacgcagtggtcagatgacgagctggccacaacggcgctggctgcgtgcttcagcgatgccatccacaaggcaccgaatccggtcatcg |
| | | ttccggtgctggagccgcttgtgcttggcgaggatcagcttgcggccgtgcgtgccgggctaaacgatcggctgaccgtggtaaccgggccgcccgggaccggcaagtc |
| | | acaggtcgccgttgccctgatggctagcgcagcgcttgtcggtcgcagcgtcctgtttgccagccgcaatcatcaggcgatcgacgcagtcgtcgggcggctggccgaag |
| | | tagttgaagaccggccgctggtaatccgtgccaatgcgcgcgaaagcgatgacagcttcgactttacccgtgcgatcgaagccatcctcgcgcggcccggtggtgagagg |
| | | cccggcgaagggctggctggctcgatcgaagtgctgacgcggctcgatgcggcacggaccgctgcgatcgaacaggccgccactgctaaccaagcgatcaacgaact |
| | | cgggcggctggaagcagcgatcggagatctgacggcagcccttggcatcgacgcagccgctccactaccgcgggatctgcccgctgccacacgacccttgcatagttgg |
| | | ctagagcgcctgtttgcgccttgggtacggtaccggcgactacaacggctacggcgtctagcgctgggatggggccagcttggttttggcgagtgcgacgaatcgacgct |
| | | ggagctacacgaacaacgtctactcgacctgcaggagctggctgcgctgcgggtcgagcgggatcaggcagaggcagccgtgcgtcaactccgttcaaccggcgatcc |
| | | gatcgcgctcggagagcggctgtgcgcttcatccaaattgcgtctgcaggggctcgccgaactgcttatcgagtgtgcgcctgaagatcgccgtgcgttgaccgcgttgcg |
| | | cggcgatctggctctggcgcgcggtgatggcgccgccggtgctgcccgtgctcgggaactctggtcggctcagcgagccctgatcctcggccagatgccgctatgggcc |
| | | gtgtcaaacctcggcgcagccagccgcattccgctggtacccgggttgttcgattatgtggtgcttgacgaggcatcgcagtgtgatatcgcttcggctttgccgctgctggc |
| | | ccgggctcggcaggcgatcgtgattggtgatcccgcgcagcttacgcatatctcccaagtgcgccgggagtgggaagccgaaaccctgcgcaatgccggcttgatgagg |
| | | cctggcatcggcagctatttgttctcgaccaacagtttgttccatcttgctgctgctgccgccggcgaccatcacctgctgcgcgatcacttccgctgccatgaagatattgccg |
| | | actacattagtgccacattctacggcaatcgcctgcggccattgaccgacccgcgtagcctgcgggcaccagtcggacaggcagccggttttcactggacgaccgcgccc |
| | | ggtccgatccaaccagcccgcaccggctgctttgcaccagccgagatcgaagccatcgtgcacgaattgcattggttgctgggtgagggcggcttcactggaagcattgg |
| | | cgtagtcacatcgtttcgcgaacaggccaaccgtctacgcgaccgcatcgagcattgtttgagtgccgaggcgattgcaagcgcacgattggaggttcacaccgctcacgg |
| | | cttccagggcgatgcgcgcgatgtgattctactcagtttatgtatcggtccggatatgccggctggggcgcgagccttcctgcacgacacgggaaatctcgttaatgttgcgg |
| | | tgagccgtgcccgcgccgtttgccatatcttcggcaacctggagtatggagctcactgcggtatccggtatgtcgaggcactgctggcacggcgccatcgaacaggcgatg |
| | | ccactgccagtttcgaatccccctgggaagaaaagctctggcgcgccttggctgagcgcggtatcgagacaacaccacaatacccgattgccggtcgccggcttgatctgg |
| | | cattgctgaccgacagtgtgcgtctcgatattgaggtcgatggcgaccgttttcatcgcgacctcgacggtcggcgcaaggtgggtgatctatggcgagatcatcaattgcag |
| | | gcgctcggctggcgggtcgtgcgcttctgggtttacgaactgcgggagaacatggatggttgcgtcgaacgcatccttgtccacatccgaagcaccgattactgagcatcac |
| | | cgttccccaccagcagcagccgtgccaccagcgaattggcggcgaatgcaactcgtgctcgggctggccggggctctggcgctggctagcctcgtcactgtattggtggg |
| | | tgtaatcggcgacgccaccgaacgcgagagttggcgagtacggcgtagcgagcatcaggaggtgctgggcgcgctcagcaccgcacgtgcccagcttgatgaggaagt |
| | | cgccaacctacgccgtaatcgtgctgcgctcgatgcagacctgaatcgtctccggaccagcgccgaagctgagcagggcggcgcagcacggctgcgtgaggaagtcgc |
| | | cgcactacgccaggagctcgccgccggccgcgccgagttggctgtggctacgcagcggcgcgacaccctgcaggctgcagtgaagacggccgatacgacgctggcg |
| | | gaactgaacgcgcgccgcgatgaggccgagcgtcagaccggtgaggcagcagaacgccggcgggtcgcggccgaagccgagcgggccgcgaaggcccagcaga |
| | | gcaaggccgaacaagcccgcgacagtgcggttgcacagcagaaggaggctgagcggcgcatcgagcagatccttcaggacctgaaaaccgccgaagaacgagtagg |
| | | tggactgcgcacgcaagaggctcaactaaaagcggctacaactgcctccactgccgaacgtgaccggctggatgctgaagccaagcggctcggactggagcttgtcaag |
| | | ctcgatcagcagcgccagcagcttgagcgcgatacccgtactaccgccgaaactcgacggacggccgaggggctccagcagcagctcgaccaagcgaaccgggatct |
| | | cggtaccgtccgcgaagccctgaagaccgcgcaggggcagctagccgaaacgcgcggccagcagacccaactcgccgacgaactggcccggctgcgcgcacagaa |
| | | aaccggcctggatggcgtgatcaccgcggctgctaacgctcaagcggaacttgacaaactgcaggctcagcagaaacgggcggagcaagcagcagaaacgacgcgtc |
| | | tcgatgttcgtcagctcgaatctcggaaaacggcactggaagccgacatcatcaaattcaccgccagcggcaaggatttggaaaagttccgtgccgaactggctgatacca |
| | | atgcagaactcgaacgtctgcgtcagcaattggttgaggcacggagccggcgcgagactatcgcgattgaagtggaacgcctaacgcaacagcgcggcgaactggagc |
| | | gcaccatcggttcactaacgccgcgagcgcaggaggccgaagcgctacggatccggctccagcaagacaacggcactttgctcgccctgcgcgagcagattgaacgctt |
| | | gcgcactgaacgtgacagcttgcagcagccggtcacatcttccatgcatgtccccggcgacaacgccgcggcacgctgatcaaggatcgcgctgatggacacgaacacc |
| | | ctggtctggcttgcatcgggtggcacgcttgccggcatcgtcagtgttatcaccgcattggtgtgcggcatgcactacggtgcggcgctacgccgcataccggctgcggcct |
| | | ttttggaagatatcgtcgcacgcgtcgcaactcgtcgcgaggaactcgaacggctggatgcccaattgggcgagcgccacaacggcctccagggcctgcggggcgaaa |
| | | cggagatgctgacggcccgccgggatgccttggcagcgcaactgcgcgaactgcaggaggacctggttgcactcgatgggcgccgggccgacatcgcttcggtgcgc |
| | | gatgagttggcggaagcacggacgcaacttgccatgctcgtcagtgaactgaccgaacggcggacgcagcaggagcaactcgaacgcgcggccgaacgtgcccgtgc |
| | | acaactgtccctgctcgaagaacgccggagcgagatcgaggcaatcgatacagccgagcgcgaagcacggatacggctcaccgaggcgcagacggaactgggcacc |
| | | gtcgtccaggcgcgggaagcggcacggcgtgaagccgaggcggcagcgcgcgacagggagatgctggcaacgaacatcgaccggctcaccgatgagcgcaacga |
| | | actgcgcgctgacatcgccagtctccaagccgaacgcaatccgctgtcgactgaagttcagggcctgcgccggcacttggagcagttgcatcttcagcagcaggcactcg |
| | | acggcgatcttcaacgcctgcaatccctacagccggtactggaagataaaatcagcggcctgcaacaggaagttgttacccggaccgctgaactcaaagaccttcaggcc |
| | | gaacgtgatccgctgtcgactgaagttcagggtctgcgccggcacttggagcagttgcaccttcagcggcagacactcgacggcgatcttcaacgcctgcaatccctacag |
| | | ccggtactggaagacaaaatcagcggcctgcaacaggaagttgttacccggaccgctgagctcaaagaccttcaggccgaacgtgatccgctggcagcggacattgatg |
| | | gcctgcgtcggcaactcgaaccgctgcgtacacagtgcgacgaagtcgaagcggaactcgcccgccgccgcgccgaactcgccgcgatcgagcaggagatccgtacc |
| | | aaaggcggtggtagcgtcggcaacccggaagacgtgctcgccgatctcgaacaggcaccggcttgtctggtcggcgacggcggcaggggaccgttgatgccgaatcc |
| | | gcagcgcgacgacgacgaaacagcaatgctcggccgcgtgcggacacaccttgatcggctccgtctgcactttcccgagcgcactctttatgcttttcatactgcgctcaag |
| | | acggcaacgattagtccgcttacagtgctggccggcatttccggtaccggcaagagtcagctgccgcgccgctatgccgaagcaatgggtatccatttcttgaaactgccgg |
| | | ttcaaccacgttgggatagcccgcaggacatgctcggtttctacaattatttggagaagcgctacaaagcgaccgaatttgcacgggctctggtgcatttcgacacgtacaact |
| | | ggccgcttgcccggcctttcaaggatcggctactgttgatcctgcttgacgaactgaacctcgctcgcgtcgagtactacttcagcgagtttctgagccaactcgaaggccgt |
| | | cccgccccgggcgatcgcgatcctgagcacatccgcagttcggaaatcgtgctcgatactggcggcgttggcggaccgccgccacgcatctatcccggccacaacctgc |
| | | tgttcgtcggcacgatgaacgaggatgagtcgacacagacactttccgacaaggtgctcgatcgcgccaacctgctgcgcttcccgcgccccgaaaaactggccggaga |
| | | aacgctggcgagcggcggcgagccggcggaaggcttcctgccggcctctcgctggcatgcgtggcggcgcagttttggcacgctgccggcaacgctgcgcgaaccag |
| | | tcgaacgttggatccacgatctcaatgagcatctagacgggctgcatcgaccgttcgcgcaccgtgtcaatcaggcgatgctcgcctacatcgccaactatccgggtgtcgc |
| | | cgagccgatggcgcaaaccagtcctctggatcaggcccgcattgcctttgccgatcaactcgaacagcgcattctgccgaagctacgaggcattgacctgggtgactctgg |
| | | agtcacccagcacctcgaccgcatccgtgcgttgatcgacaacgagttgcatgatgcaacactggctcgcgcctttcagcgcgccgcgcaagatgacggcagcggcagg |
| | | ccgttcgtgtggaaaggcgtacgccgtgaatcgatatgatcccgctggtgctggctatgccatggggactactggcacagactccgatcgccggccagccgacgcgccga |
| | | ccgttacatgacggtgaaacggtcgaactcgatgggcggtacggtgccatggtggcgctacccgagcggaccgacctgcaactgggcagtcggcgctggccggtgcag |
| | | gtggaaggtgccgcctttgcctggttcgagggatcctttcggttggtgtcgctgccgactgcagccttgaccagcgaacgtcagatccggttcgatcttctaacggcgggcg |
| | | agtctgtgctgagtgtcgggctcgtgttgcgtaatcatctactgcgtccgcgcggagccggacgtgacgatccggccgccgatgcattgcacacctttgtgttgcaggttctc |
| | | gaccgcatccgtgaggccgaaccgtccggtgccggagacgattgggatgatctcggcaccggttgggcgcggctgcgcaccgcctggcttgagcgcgatgcgcagatc |
| | | gaagaagcgcgccgcgatctgatcgtcgaacatgctgaacaactcccggcccacatcacagaaatcgctatccacccgcgtcgggtgctcaaacgcacccgcgagttgct |
| | | gccgatcgatcgtatccaggaactcgacaccgcctgtctcgaatggctgatccggcagcccggcgttaccgttgccgaaaaggccggtccgcgccagcgactgctcggc |
| | | atcgcgcgcgaggagcatctcgatacgctcgaaaaccgggtgctgaaagatttcctgcgtctgagcgtcgaggctgccagcgtctggcagcgggagaaccggcgttttca |
| | | caacagtgagcgcgcccggctggtcgggcgttatctcgcgctgtgccgcatgcatcatcgcgaactgtgcgcggctggcatcggtgaccccatgcccccggtcgctccga |
| | | atttcgtgctgcaacaagattcccgctaccgcgtgatctggcgcgcgtaccgcgaactgttgagcgctgagcagcgtatggacgatctctggcgctggcagtgtcggttgtg |
| | | gagcgacttcgctcggcttgtcgtggtgatgggggtgcaagagttgtgcgacaagccgagtgcgctctcgcccctcttcgtgcgcagggaacaggcaagcggacgctggt |
| | | cggacacgctcggcctgctcggtgtattcctgatcgacctgaacggcaggtcgtatgtggcggaagtctgtgatgcgagccagttgccccgaaacgacacgtcacgagcg |
| | | aagctggcgtcctggcagtatgcactcggttgcacagcactcatccgcctcatcgatttgtggagtgggcattgtgcgagcctgtgtgtctgggccatgcatagcgctacagc |
| | | cgagacgcttccgttgaccgagttggtcgcttcagccgatgaagccctgagtacggccatcagacaggaaggtctgcgcaacggcgagcaacttcgggcacgtggactg |
| | | gtgatccgctcggcgccgccgggaaagaccgagtacgccacccaggctgggcaggtctacggactgacgctggccatcgggtcggaacatatccgcgaggcgcttgg |
| | | cgagtgcactttgatcctgcaggacagtctggagcgcctgtttgcatgagcggagtgcacggcattgatctcaatggtgtgctcgattgcgtggtgcgcctcgatcgggcac |
| | | cgcgaccagcgccgacaccgccggtgatcgtctccggttcaccacagggcctgctgacgggagccgcggcactgcaatcgccctgcggccgacctggcatggaagcc |
| | | gaggaaggtatccgcctgccagtgctggccctgctgcacgcgctcagtggtgaggggcggcacgatacgcacgatacggccgtgctgctcggccgacacctgcgtagc |
| | | ctgttgtccgatgatacgcatgctgctgtcgtcgcagtgcctgacacacctggtttcgacgaacgagctcgcacccggctgctggatggcgcgctacgcgccgggctcgat |
| | | ctgcacctactatggcgcccggtcgcagcgttgcttggttggggcgaaacactgggaaacggcgaactccaagccctgcacggccggacggcctgcgtcgtgcagttgtt |
| | | gccggacggcatctcgattggcgatttcggcctcgaatgcgtggtgcagggtggccggccgacgttagtaccggtgcgccggcgcgacggcgaacgtcaattttactcgt |
| | | ggagcggtggtggactggttgcactgctcgcgcgcgaagctggaaccgacgaagccagtctgtgggtcggaccgtgggtatggaaggtcttgcttgggcagcctgcaga |
| | | acgcgaggtgctggccgacccgcatgcaccgggtggttggcgactcgccagcggtccttccacactgtgcggcgccttagccgcggagttgcgcacaggcctgcgtata |
| | | gcactcggagccgcgcgctcggcactgcgcaatgcagcggtcattctgatcgaggggcctatcgccgatgcaccgcttttggacgcaatgcagccaacactcgcgctac |
| | | gccagatcgtggctgcggaactgaccgtggtgctcggcccgacggtgtccgcaagactcgtcgccatgccgctcgccgatgctctaattgccagaggggccgctatctgt |
| | | gctgcgcgtcaagcggcgcggcagatcacgtattacgatttcctgccgatgctcgaaatcaatgtgctgcaggccggagagcatgcgttcgttgaactcatcggtcgcgaa |
| | | gagcgcatcgcggggggcatgagttacacgaatacgttggccgatcgcttcaccgttgccgcaagcacgcgctcgctcgagttctacctgctgaaagaggacgaagcag |
| | | gcgctcgtcacagcgaaacggtgctgccggtaccgccggcagccgacgtggaaatcagcctgcacgtcacgcagacacccgctcaaggctacgcacgcgtggagata |
| | | ctctcggccgtccggggcgcgctcggtgaagcaccgatcctgctcgattggtcagcgatgacagagattgaaggctcgcgcgaggatattctgcgcgaactcgaattcga |
| | | ggggctcggctatccggacatcgtaccgcaacgtgcacatcacctgctctgggattaccagcgcagtgacggcatgactatcgctgccgcgatgcgggccttcaattgtaa |
| | | gcctatcctaagttcaccgcgcaaccagtacaatcaattggttaaacaaacgcgcgcactcgtcgggctgcgcagcaatctgttttttctgacaaagggcaccagttctgatcg |
| | | tagtgcttacaccgccgtcgattcggatggccaattgccacctggaatcgcgccgacaatccaacaggaattcgaaaactttcgagtgcggctcgacacggattttgccgca |
| | | atcaccagcgtccgtaatcgacaagatatcgcaacccggcgtgaattggcgcgactgggcgcctgcttgtatgcagcgtgtcctaatgcaattgttcattacttccaacgcatt |
| | | gtcgcacgtagcgccgatgacctgacactggtgttgcatgccggcaaagtgctgagcaccgaaccagatcttgacagtcttttccattattgcgcgtctcgctacgatgaagc |
| | | catccgcgctgtcaagagactgtcggtccacgtggtacgcgcggcaggcgatgctttggcttatcatgaaaaagctggaggcattcttgataaccgaagcgctgacaagttg |
| | | gctgaagctgcgctcctattgctaaaggaggaaatccaggcacataattacaaaatacgattccgtgccgccgcgcgactcggcctatttctgttacgccaccggcagcggc |
| | | ggcgcgatttcctgcatccgagtagcgctgacacggctaatcgtcggcgtgccaaagagttcgatgccctgttgatccaggctatcgcatcgaagcgccttaaccaagatct |
| | | ggaaaatgccttggaagaaatccgtgcacaaatccgatatcgcggtacaaatgcgatcgttgatatcgatcctgacgaagatggcgagattaacgagaacgaagtggagta |
| | | gaggctgttgggcacccgctcgccatccctgtcgagcatcccggcttcgcgggcgcccatcccgtgcctttacggcgtgttcaacggccccggttcgccctgcgtatcggg |
| | | ctcctgctacgcccgtcgagacgcgctgcgcagactcgacgctcaaatggcttgacgccattctccctggctacc (SEQ ID NO: 404) |
| 41 | Helicase- | atgtctctggttttgaagacggttcgggcttttccgagaggtcggactaccgaagaattgcttgttctcgtcggtgcggctttctcaaatgacaagcggcttgcggctctcagcg |
| | DUF559 + | aactggagacgctatttcgcgatggtttgatagtgaaaggcaaggacggtcgctggcgtgcaaaggcagatggtttcaaacccagacatgagagcgtgtcggcttcgaga |
| | SMC + | ggtggagggcctgagggcttcgttgatgtcattcacgctgccaatgcattcttctcctcggaaccgacggcggccgaactacctgatcaagaagacgaaagttcagatgctc |
| | McrB + | ccgatccgcaagcgctactgagatattggcgctcggccttgcgtgccgatccacgaggagccacgacccaggttctcgacaaacatggaatcgagtgggccttgatctctg |
| | DUF2357 + | ggcgtggccctatcggtccagaagaagggcaaacgctgactgtttcaatcgaactcgacgcgattgatcctgcctttcgagaggctctggtgcgaagggaaggtcacgag |
| | ATPase | aacgcgcttgcagtgggttggccgatggcggtcggacgacgtggcggagttcctgtctttcgacccgttggcatgttagcagcagcttgggatcgtaaggatgaccgtctaa |
| | | tcctgacgattgatgccgatgacgttttggtaaaccctgattgggtcaaaagtgccgctcgtgccagcggctggaagcgcgacgacctcgctgacctttttttcgtggacgatg |
| | | ggctggggctgcgggctcaggattttgtggagaaggtaaggattgccgttgccagtcagatacgtggtcgcgttgtcggcgagaatctcgccacacagctcgatgcctcgg |
| | | ctcaagggatttttgacagcgccgcgatcttcctaccgactgactcttctttcaccgcgggggctgctcgtgacctggatgccattgcgacatggccgaaggaccgccttgag |
| | | agaactgcgcttggcgcggtattcgggtttgaccttcaagacggcacggacaaggctgctgcaatcgacgcagttccgctgaacaaggaacagttgcgcgcggttcgatc |
| | | cgcatgccaagcgcctttgaccgtcgtgaccggtccgcccgggactggcaaaagccaagcgatcgtatctatggccgcgtcagtgctcgcagatggtggcagtgttctcgt |
| | | cgcctccaagaaccatcaagcgcttgatgctgtggaggaccgtcttggctctcttgctccggacgtcccattcgccatccggacactgaacccgaatgacgaggcggatac |
| | | gggcttcaaggacgccctcaaacaactcatcgacagcgaaaatgtgacgcgcaacgcatctgtcgacgaattcgcattaggcgagctcaaaagcgacgcgatcgcgaga |
| | | agcgaagtggttagcgtgatcgataagatcacggaaacggaatgcgaaatttccgatattctggaccggattcaagtccgagaggatcgcgggcgccctgacaaccaaga |
| | | ctctgaagacgtggatccgagacaaagtctcttactccgctttgtctcttggtttggatcgcttttcgccaagcgtccccccaaagtagcgccagtgacagatcattcttcgtccc |
| | | gccgcggaatgaacgtcaaagagcttcattgcgcgctggcagaaaaaagatatgaacgcgatgcgctcgggacacctgacgatccgatcgccttaggcgagaagatccg |
| | | ggaagcgaccgagaatcttctgcctcgcattctgtccgcccggacacatctcccagaggatgagaggcgcgaaatcgcagaactctacgatgactggacattcgacgggg |
| | | gacggggacatccccctactgatctttcgcgcgtcctcatttcgcatcggcctttgtggcttgcatcgatcttgggcacgcctcgacgcatacctcttgatgacgggctgtttga |
| | | cctcgtgatcttcgacgaggcgagccaatgcgacatcgcgacggccgttccgttgctggcgcgcgcgaagcgggccgtcgttgttggggatgatcgacaactgtcattcat |
| | | ccctcaactgggtcaggcgcaggatcgcaatctcatgcaggctcagggcctaccggtcgccagaatgggccgtttcgcccagagtcgccgttcgctattcgatttcgcatcg |
| | | cgcgtgtctgttgccgacaacaggattactctgaggcaccagtatcgttcagcaggccccatcgtcgattacatcagcgagaacttctacggaaaccagttgcagacctcgta |
| | | tgacccgaggcgactgaacgtgccagatggggtgcgccctggcctcgcatgggaacatgttcctgctcccgcggtcccgcaaatgggcaacgtcaatccgtcggaagta |
| | | agcgcgattgttaggcacctgaaaaagctgatcgttgaagacaaatacactggcagcatcggtgtcataacgccgtttcgcgctcaagtggccgctatcgagaacgcggtc |
| | | gatgccgtcctggatgaaccgaagcgcattgcctgcgagctcaaggttggcacagttgacggttttcagggacaggagcgggatctcatcatgttctcgccttgcgtcggtc |
| | | cacgcagcccgcagtctggcttgaccttctttcagcgagatacgcgccgtttgaacgttgcgatttcgcgggctcgggcggtcgcgatgatcttcggcgatcttgattttgcac |
| | | gttcagggcaatcaaaagcgctggccaagctcgcttcgagggcgacggaagcgcggacgaaacggggcgaaggtgtgttcgacagcgattgggaacgcaaagtctatc |
| | | acgctctgaaggcccgaggtctggatccgcagccgcagcacgaaatagctgggcggaggctggacttcgcgttgtttggagcgaatgatgtaaagctcgatctcgaggtc |
| | | gacggacgcagatggcacgaaagcccagacggtcgtcgaaagacgtcagacctgtggcgcgatcatcaactgaagtccatgggatggcgggtgcgccggttctgggtg |
| | | gacgaactttcaagggatatggagggttgtcttgaccgagtcgaacaagacctatcgtaagtcgagcaggaacaccgcggttgcgttggggctgggtggcgccgccatcc |
| | | ttgcctcgggctttctcgtcctgcaagtcaactcgctcgatcgccgatatggtcgtatcgaggaaaatctgagctactacaccggggaactccaatccgcgcagcagcaact |
| | | ggcttttgctcgtgagcagtttcgcgaactttctgaccaaaagcaaagcttgtctcaggaagtcgcgagcgccgaacgcagccttcaaagcgcggctcagagagaggcgg |
| | | atgcgcaggctagtgtcgaagcaagccaggccaaattgactgctgagcgggaccgtttggccgaagcccaaaaaacgattgcggatgcgcagcgaattgaacgtgaaac |
| | | tgctcaagctttgctgcgaagaaatggcctcgaaacagaggtggtcaaactgaaaggcgatgtgcaggcccttaaggagagccagcaagagttgtctgctggtgttgacca |
| | | aacgcaatcggctgtcgatcgcctcgaagagagaagagctgaacttcaacgtgaagtggatagactcgcgcccgccgttgaagaccttcgtgcacaggagcggcttgtcg |
| | | aacaactgcgaggtgacgaggatcgtctcgaacagagcctcgacgatttgaatgcgaacattgcaattgcacggactgaattggcgaccagcgcggaaaaggtcgatgc |
| | | ggccgaggagaggctgcgtgcagggcaggaacaaatagcatccacagaagctcaacttgaaacactgaatttcgaagtcgatgacctcgagtcgagacagggcgaact |
| | | gcaggcaagtgtctcgggagcagagacgcgtcttttttcattgcaaaatgaactggagatcgcacagaacgcggtgacgcgagctgatgcgcagcgcgctgaaactaca |
| | | gaagcactcaacatcgctcaggaacagttttcgacgcgaagcgctcagctctctaccctccagtcgcagattgcatcggcagaggaagagcttgccgaacttgaagagag |
| | | acgggcggaattcagcagattgcaggctcaaatggaccagctgcaagcacgtcgaacgacactagaggaggttctccccgatcttgagaagcgagttcaagcagagcgg |
| | | gctaatttgggttctatcacgacagaagtggagacagagctcgggcgagttgctgtactcaaaggccagggttccagtctggaggccgacatcgagcgcctccaagagcg |
| | | tcgcgacgaactcgggctggaaacgcagtccgccactgctgaggcggaggccgcgcgcgcatcccttcaagctgagcttggtcaacttgcggaaaccgatgccctttcaa |
| | | gagcgcggactgccgatttgaggcgcttgagagaagctcttggagctgctgaaagagagctttccgaacttgaagagagacgggcggaattcagcagattgcaggctcaa |
| | | atagaccagctgcaagcacgtcgaacgacactagaggaggttctccccgaacttgagaagcgagttcaagcagagcgggctaatttgggttctatcacgacagaagtgga |
| | | aacagagctcgggcgagttgctgaactcaaaggccagggttccagtctggaagccgacatcgagcgcctccaagagcgtcgcgacgaactcgggctggaaacgcagtc |
| | | cgccactgctgaggcggaggccgcgcgcgcatcccttcaagctgagcttggtcaacttgcggaaaccgatgccctttcaagagcgcggactgccgatttgaggcgcttga |
| | | gagaagctcttgctgctgccgatgatgagctttccgagacacgagcggaactgatggacggacagtctgtggaacaggaaccagtatcaaccattagtgaaggcgctggc |
| | | gcccgtgaaaacgctcagtctgacaactccgcgccatcgagcaccgacaattgaggtaaccgaaaatgcttacggacaatacaatacttgtgctggcgattgcgggtgtcct |
| | | gatactgctcgccgtggttcaactttttctggccgcccgccacgaccgggcggttacggcagcaggcccgatcgaagagcttgccgtctacgagaagcggctggaagaaa |
| | | aacagcggctcatggacgatcttgaagctgaagtggaaaaacgtcgggaggcaatggccgtcgttactgacctccgggctgaggtcgacggtctacggcgtcagaagga |
| | | ggagctccttacagaatgggagagtctccgtgaacgtcgcgacgaagttgcggcagttcgcaaggagactgaggacgccgttgtcgaacgccagcaactcgaaacgga |
| | | gatcgccccgcttcgtgcggagtatctggagataaaggaaaggctggaaaaggcggaggagctcattgagcgcactgacgccttgagacgagagcacgacgaaatctc |
| | | cacacaggtcaaagatcttcgggacaagaagaggcaacttgaagaggccgaggaacgggtttctcgcctggaagagcgttccttcgaacttgagacatcgaatgctcggc |
| | | ttgagggacagaagtcttcgcatgaaagcgagttgtccgccttggaagcgcggatcgcctcggaacacggtgggttggcatctgcccaaaccgaacatgctcgcctcgat |
| | | gcagaggttgcggctctgaaccaggaaacccgccgctccaggggcgaaatcgagacgctccaggacactcgaagcgcgcttgatgctcgattggcacacctcaaggcc |
| | | gagatagctcgccgagaaggtcgaaccgtcgacggggaaaccggcgaaacggatccgcttcgcgagctcaatgaaacaccaccggtcattacggagatgaggacctg |
| | | ggacaacgcgccccgcgagaacgaggcggatgccatcaaacgcgtcgaacgccgcctacgcgcaaagggtctcgactacccggctcgcacgcttcgcgcttttcacac |
| | | cgccatgaaagtaaatgaaacaacgcagatggcggtccttgccggtatttccggaacgggcaagagccagctcccgcgtcaatacgcggccggtatgggcatcggtttctt |
| | | gcaagttccggtgcagccacgttgggatagtcctcaggatctgatgggattttacaactacatcgaaggcaagttccgacccacagacatggcgcgtgcgctttgggcggtc |
| | | gacgggcttaacaacgacgatgcggaacaggatcgcatgatgatgatcctgctggacgagatgaacctcgcaagggtcgaatactatttctcggacttcctcagcaggctg |
| | | gaaagccgtccgcgtcccgatgacgtcgacaatgaaaacgaacgcaaggacgctgtgatcgagcttgaaatcccgaacatggaacgcccccccaggatttttccgggcta |
| | | caacctcttgtttgcgggcactatgaacgaggacgaaagcacgcagtcgctatccgataaagttgtcgaccgtgcgaatatccttcgtttttccgccccgaagaaaatcaagg |
| | | acggacaggcagaaggaacggtcgagccgattttggccctttcgcaacagacatgggagagctgggggcggtcgagtgcgtctgtcgatggcggtcggcgtgtcacca |
| | | accggattgaacaaatggttgatctgatgcgtgacttcaaacggcctttcggtcatcggctcggacgcgcgatcatggcttacgcggcgaactatcctgaggttgaaggcgg |
| | | ccgcggtgtcgacgacgctctcgcggatcaattagagatgcgccttctaccgaaactcaggggcgtggaaaccgacatggctggccctcagttctcgaggttgatgaccttt |
| | | gtggaacgcgagctgggggacgacgccttggcccaagcaatcggtgagtcaatgtccctcgccgaggcaaccgggcagttcgtatggagtggagtcacgcgttgatgcg |
| | | gtttctggcccgtccctgggcggcgaaagcccttggagaggacgaagcctttgggcccgaagactgtctgatcggtagctaccagggggcgaacccaggcggctacga |
| | | atacgtgacgctcttgaggggaaacgtccgaggtagcgataccggaactgttctgtttccctatccaaagcgtgaggaagctgtcgggcccgcgcgtaagggcttcccggt |
| | | gcgcccaaggtcggggcacgatcctgccactccggacgaagaagaaggcgcagaggcccttcgacacatgaacgaagttcttgcacgtatccaagaactggaaggtgc |
| | | gattgaagacccaagcgatacatgggggcgcctgagggatgcttggaagcgcgccgaaaatgaagccgaacccaaaatggctgaaatcgtccggcaggcgcggggca |
| | | tgcttccggtgcttcgcgatctggaaaaacgcatccgccgggttctacgtaggcacagggagctaactccccttgatcgggtgcaggagatggatcggacctctatggtgtg |
| | | gctcagccgacagccagggcgaagcatcgcggaacgtgcaggttcttcgcaacgaattcttgcgacggttcgccgtgagaatttcgatacgctcgagaaccgtgtcctgca |
| | | tgcctacacgcgtcttgccgcagatgttgcacgcgaatggacccgtgagcaccctcgtgcgaaggacagtgttcgctacaaacaggttgaggcttttaggaaggcctgtcg |
| | | agtattgtcgcgaacactcagtgacctcggtgtcatgatcgcgtcggccggcgtccagccaaactatgtgctcatgcaagatcgcagctatcgagaggttcatgagggatgg |
| | | ctgaggcttctcttacgccgaaaaattgtagatgatctttgggcttggcaggccgaaacttggacggatttctccgttctttcgatcattcttgccatcgacgaattggaagaggc |
| | | tgaacttgtcgctcagtcgccgatttcgtggagcggtgaggcaacaggcggacgctggttcaatcaggatcggccaatcgccgtcttttggctgcgcgacaccaaccgcatt |
| | | gttgaagtccaagcacgccctgagcgaccaggaaccatgttgagcgcggcacaagcgcacgtcgccctcagaatttccgatcccaaacgggctgaccttccgcgcagga |
| | | tcgctgtctggacgccacatgccatgcgtagaattgatctcgaggatactgtgcggggggcagttcaactgcttcaccaaatccagcccctcgctcagacggaagttttgcg |
| | | gaatgggttgatcatgaccccagcacgtggtgtcgcagctgaagagagcgcaactcacggaagagcgatcgttacggcaatcgccataggcccagccggtgaagaccta |
| | | gcgaagggattccaggccgtgcgcgacttcattcgcagtgagctatacgaggtcgcaacatgatcgaccgaaaactatgcggcttcgatctcaacggatggagagatttcg |
| | | ttgcgaagaactggcgctccgtgccaggtgaagacgaggtcattggtccgaccgatatcgtcacaagtggccctattcgtcgatcgtgcggatcggggaaagccgcctcg |
| | | caggttggatcggaggaccgcaggctgacattgctccgcacggtcgcggtggtggttggggtgatgtcgggtcagaacaaagacgcattcccgttcggtcactgctggaa |
| | | atgcgtgatgacggggtcgaaaaactcgcccaggcacttgtgggatctgcgagcggttcggcaaacacagtcgtttcgatcgatgagggcccggatggcgatgaagccgt |
| | | ccaagagcaccttctcgaagcacttgcccgagggaagttccgaaatggctcattggtttggcgaccagttcttgccgccttgttcgccattcatcgcgatcaggtttcggagg |
| | | ggcagcttgtaggcgtcgtctcccatcagcgccaaggcttgtcagttcaaaagctgcgtattcgtagcgcaaggaatgtgctcgccccggagcgacgcgaggccgctgcc |
| | | catataccgtgcgacgctggttacgagtccctattccgaggtgcccgcaacgccgctgtcggggcagagggtttttcggcgcgcacagctcatcgtgcgatcgcaagctcg |
| | | gtcggaaaagctggtttagggatggattgcaatcctgagatgctccgcatgcccaacggcgattgggagctcttggaccttaataaatttgacgcgtcggaagtggtgagtgt |
| | | cccgagttccgagctcgatctggccgattgcgacgtcgttcttttcgagaccctttgtgaaggtcggctcaaaaaatgcctgagtgatgctatccaaagagcagctccagtcg |
| | | aggtgctctctcttcccgcaacggctgttgcggaaggtgccttggaagcagcacgccgagccggggacggggaaccgatcttcttcgactttctaccacgattgtccaccat |
| | | cgtgttcggatcggatggcgcaaagaatttcgatctcatacggaaagaagaaacgctcgaagcaggccggacctacagaagccctgaagcagcatctctcgcgataccg |
| | | gcagggcaggagagcgtctctgtctacctgaggaaagaggaagctccctggcctcgaaaggcaagggtgtcgcttggagctcctctgaagcatcaagctgccgtctcgct |
| | | gtgggtcgaacagaaaccggccgccgggcgagcgcggatcctcatggaatcgccggacttggggcggaatttcgcggtggattgggatgaagcactggaagaggaac |
| | | ggccctggtctgagatcatcgagagcttggatacgcaagtgtcaattcccaaacgtctggttcttccctgcggcatggaggcatggcatgacagcgatcgatccgcaggtat |
| | | gctaactttgctcgaatccgagcctaatcgcagccgcacggattgggcgacccttcggcaaaaactttcacagcgtccctttggcaaatactgcatctcaagtgacggcgac |
| | | gtgcctccggagatcgcggcagaaaccctcgagcggtttgaaattctgaccagcaaagcgcttgaggttactgaaaagcgcctgaggggcgaaagcggctacggaacg |
| | | gaagacaatgaggctctcaaattcttgagttggcagttccgccgatgcccgcgcgatgtcgcgacgtggctgatggactgtattgaagcgtccgggcgcaaccatccgttcg |
| | | tcaaacatcaagcaagttgggttctcgtatatcagggccttggccgcatcgtcggaaacgaagaggacgaagcgagagcaatgcggttgcttctgacttcgtccattgagga |
| | | ctgggtctggaaccgacaaagcgcggccatggcgttcatgctgtctcgttctgacagcgctccatcttacctggaacgagaagacgtagagaagctgaccaagaggactat |
| | | cgcggacttccaacgtaatatcggcggccaatatacaatgtttaactacgcgcctttcttacttgcaggcctgataagatggcgtctcgttgatcctaaagctttggtgatcggg |
| | | gccgacccgttggcggatgacctcttggctatcattgagaaaacagagcacgacctgaaggcccgttgtgggtccaatatgaatttccaaaggcggcggtcgaagttcttgc |
| | | ctatcctccaagacctgaagtcagagctggcgggagaaggttcgaatcctgacctgttgttggatatctatggagcgagcggaacgtga (SEQ ID NO: 405) |
| 42 | GTPase + | tcgcgatcaaggggtgagcaggggataaacgcaaagacattgaagttgaggagaatttagttgccttacctgcgaaaaatctgagcgatcttgcattaaagattttctatctca |
| | GTPase + | ggccgatgctcataagagcatttcctgaatttcaccctttttttgctcgccatccctctgcgaataaggacaccgcgccagatatgtcactcatcacccatacattagaaaacctc |
| | TM | acaaaagccttgcgtactgcgttgcgtgtctcaattgaatgcaatgagcgcagcgaaaatacccataaaattttaaacgtgttacgtcaggttgagctgacgctgatgctgcat |
| | | caacaacctatctatgccattgccggtacgcagggagcgggtaaaaccactctggcaaaaagcctgctgggcattgacgatagctggcttgaggcgaatccgggacggg |
| | | gcgagcagataccgttatttattgagcaacggcacgatgttcagggtgattatccgcaatttatttatgtctgtgctcaccacaaaaccggtgaaatttttgacagccagccgcg |
| | | cagtggcgatgagctgaaacagatgctgcgtgactggtcgcaaatggtgaatcaggagatagaagggggcaaaatcctctatccgaaattaatcattaataagtcagacagt |
| | | tttattgatgaagagatggtctgggcgctgttgcccggctacgagatcagcaacagccagaatcatcgctggcagggcatgatgcggcatgtcatggtcaacgccagaggc |
| | | gtgttgctggtcactgacccgacgttaatggcaaatacgaaccagagcctgctggtgaacgatctgcgcagtgtgttcgccgatcgttctccggtgattgtcgtgaccaaaac |
| | | agaaagcctgaacgatgcggagaaggccgaggtaaaagcgagcgctgccgcactttttcatgagacctcctcaccggtggtcgctgccggtgtcgataatcaagcgcagt |
| | | ggataggtgagctccgcactgcatttgctgagggtatccataatagcgccgcgtcagaagcggccgcgatcgaacgtttgatgactctggtcaatgacgatgttgcggatatt |
| | | attgataacctgaatctgctgtacgcggagcaggacagtggcgaggaacgtaccgtcgctattcttgaagcgttcgataaagcagccgagcgctatgaacagcaactgcgt |
| | | aaagccatcaaacgagaaactgacgggcatcggcaaaaagccactgaatcttgccagcgccgttatcaggaagaagaagaagggccggtcaataatttaaaaggactcg |
| | | gtcgtcgtctgatgtttcagggggcggagattgatcgtgaacgcaaaaatcgggtactggacgcctggcaaacccgctttgagcagcaatctctggccgatcacaatatggt |
| | | cgcgctggaaacgctcaaccgtcgtgagttgaggcattacggtctttcacaggagacgctgtcaccccaacggttgacctcgcccgcggcgacaatgggatatttgtcggt |
| | | ggctgaggaggataatttttcctcgctggcccctttgcgccatctgctgggatcggctgcaacaagggatgcgccgccgcagttagaccagctttccacggtattaaaagtgc |
| | | tgcctgccatgacgatggaatatgcgcgcggttgggtggcgatcaaccaggcgatgcccgcagcgtcagagctaaccagcgagttgcggccacaacaaattctcgacgc |
| | | gatttttagcgcgcagagtagcatccacccggtgaaaaccgcgctgatggcgtttatcggtgccgacgccgcggacggcacgctggatggcgaagtgggcactccgcag |
| | | aatgaagatagcggcgtatttacgcctgtcgcgatagcaggcaaagcgatgctggtcggtgcggcggtttatgcgttgtatcaggtggcgggcgtggtgagtgagagtgat |
| | | aaagctcaggcctggtatattgaacggatgatgaaggaactggcgcaatataatgaaaacgtcatcatcgagcgttatcaggacacgatgggcgatctgcgtcagctgattg |
| | | aaatcaacctcaaccgtttatttggcgtgcaggatgtcctcacgcagaaaagctatctctggttagctattcagggactcacgacggtacaaaaggaagcccggcagtatgaa |
| | | gccagtatcaaacaatatctggcgtgatatttgccatgagcgttatcgatgggcggaaaatagctacatcaacctgctgcgtcaggttgatgccgagcggttaatccagcctc |
| | | atgcagacatctcccgccagatatcggtcattgtctatggtccgacgcaggtgggaaaaacctccctgattctgaccctgctgggcgtcagggatgactgttttaaagaactta |
| | | accagctgctgcgtggtgggcaggcattaggtcacgcgtcaacggcgcgaacttaccgttaccggatatcacgggatgatgcctggtattttagccacaaagaccagggaa |
| | | caaccgcctggtcggatagcggggcggcagatattttcgccagcctgcgtgcagaggttcaggcgggcaggcgctactttgacagtatcgacgtatttattccgcaacgttt |
| | | cttccatcctcagcagcggcaaaatggtttgttaatccgcgacctgccgggtattcaggctgcggatgacaatgaaagggaatatgtgactcagcttgccagccagtttattcg |
| | | ttctgcggatgtgatcctgctgaccggcaaagcggattatttaggctttctgaaacccgaggagttgggtaatgacctactggctgactggttctggcagccacatcgctacaa |
| | | aattgtattaacccggacttttagcaacagttccattcgggaaatgttgcgccgtgtttcccccgataaatcctggctgcaggcttatttgtttgagcaaatcaatacgctggaatt |
| | | gcaacttccggcggagatgcgtcaacacatttatccgctcgaatgcggtcactcctggcaaaccctgattgaggggggtgacgattatgctgactattgccaacggttgcgtg |
| | | agcagatattaaccgacctgcgccatcatatgttgcaggcggtccatccactttctcgtttacgtacgggatacgccttacctgaattaattatccgccaccgggacaagttgca |
| | | gcagcagtacacagcgctgcacagcacgctggacaaagaacaggaatattacctgcgtaaaaaagagcagctgtcgtctgtgcagactgaatattcccggcatctggcaa |
| | | agagccagacacgactggacagattgcagcggctacgggaacggctgaataaaagacaggcgcgcaacgcgcatcaatccatcgctgtgccaccgatgggcacaaga |
| | | acggtcagtgccttactgaaaatgattgctgaggcaagagaagagatggcgcttcatccggcgttaaagcaccttcctgcccatttcgctgcgcaacagattaaccaccatgc |
| | | cttcacggcgattgagcaaaagctgcatggctatcatgcggataattatctctttgccagcaactataagcatgactatcaggaaacgatcaacgcgatcaaacaacacctga |
| | | aactgatcaccacattagccgctaatttccagcgtagtgagctggagagacacatcaaggaacatcgtcgtcgccagcaacgtttacaacaccacaccacccggcgagac |
| | | aaactcctgacggcagtgaccaataagcttacgcgcatcaatacgcagcaacaggaattaacgcacagccatatgcgtgacgaggatcattatcagcagctgattggcgag |
| | | agccgtcgctttcaggaactgatcagagtggcgaaaaatgaacgagccaccctgattgaacaacacattaggcgtacggatattggtcaggctgagcgactggcctggcta |
| | | ctcgctgcccgtgcgttaaagaaagactacgaatatgtcagagcattaggagagtagtgcatgtcagtggaacatgacccggttattgcgcaggataatgacgagcggatg |
| | | ctggatgaattggtgcaggaactgtttctgaccttgctgacgcgtgagctggcgcaacagaaagcggttatcgaaaccattaatgacaacgtctcgtatcaggctggtgagtc |
| | | attaaaatcgttgaaacgggagatcaaactttccatcagcaccctgtcgaatgcgcaacagcaatatcaggaagagcaggccatcgccagggaggaatacgagaagcgg |
| | | ctggagcagcagactcaaacatttgccagtgatgcggaaaaaaatcaccaacagtcacagcagcagatggcagcacttcggcaaggtgagcagcagctggctgcacagt |
| | | taacagatttgcagcaacagcatgccacacttcatcagcgctcaggtcagatgctgaatagcattaaatggctggtggtggggctggggggcgtcaacctgctgctgtttgc |
| | | ggctgtcatcatgatgttttttctcgggcatcgataatcatccgcgcatgcaggtttgtccggatatggtgcgcctggtgcaccatgacttttctctggcacggataaacggacg |
| | | cacaggcagcgaatgacgcgccctgaataaactggcacaacttctgcattcatttcctcaggcttgtatacaaggccgcataccg (SEQ ID NO: 406) |
| 43 | TM + | atcagggcaaggaccgttgcccatatgtgactggttttggtgtcggctatgtggccaggctgcgtgaaagctactgatcgctttttaatctaagtggtggatttatatgatcaatc |
| | GTPase + | attattgataaactcatgaagaaacctaatttatttaataaaattaaaaagtatacgattagatattgcgggtgtagatatgactcaccacattaaaggtcaaggcagacatcaggt |
| | GTPase | gacgttgctctctgacgtgcttgatgattttgtcacagaagataaaaacacgttgaagagagaaaaatgaataccgcagaagactttaaccgcctctatgccgacgtttcacgc |
| | | aatattcagcagacgctgactgatatcgctgcacttcatgttgaaaatgaagagggaaagcagcagctacaatcgatggtcactcagttgcaatccctgcaggatggctttaa |
| | | ccagaagctcacgtggctgcaaaagcatgccgaatgggacaaatttaccctggcattctttggcgaaaccaacgccggtaagagtacgataatcgaatcgctgcgcatcttg |
| | | tttgacgaagaatcccgccgccagctgctgcaaaaaaaccacaacgacctggaaaaagccgagctggaattacaggaaatctcggaacgactgcgcagcgacttagggc |
| | | ggatctatagcgatgtagtggataaaatcaccgatatcagtttttccgctctgcgtctgatgcaaattctcgacaatgaaagcgccctgcgtcacaaacgggaagaggaagag |
| | | agcaaggaacgcctgctggttgaaaagacggaaagccagtcgcgattgcaaattctgcaaaaacacaccagcgccaaaacacgattaaccctgtgcattgccgccgtcat |
| | | ctcttttgtcgcaggcgcaggcgcgagcgccgccgtggtgttcaatatgatggcggggcaataggatgagtaacgcactagatcttcaggctagtaccacgtcagtacgttc |
| | | gcaacgaaagtcctcattgaatattcaggagctcctgaataaaacgctgcctcacctggttcagaccataatcaggaatgagagattaaaaaacaccctacttcaggttgatg |
| | | gtctcattatcggtaccggcgaggcggattttaccaaagggaatacccgctacgccttacatattgacgataagaccttccatctgctggacgtacccggcattgaaggcaat |
| | | gagtcacgctatatcagccaggtgaaggaggctatcgccgaagcgcatatggtagtgtacgttaacggtaccaacaaaaagcctgaaaccgccaccgccgaaaagatca |
| | | aatcatacctcgaatacggtacgcaggtttatccgctggttaacgtgcgtggatatgccgacgcctatgaattcgaagaagatcgccacgatctgatgcagcaaggaggcgc |
| | | aggagaagcgctgaagcaaaccgtcggggtactgcaaccggtgctgggctccgatgtgctgcttcccggtaactgcgttcaggggctgctggccttctgcgggctagcct |
| | | atgacgatgcgacgcaaagcaccactatccacccctcgcgcgcgcacaacctcgccacgcaacagaaacgctatttccagcacttttcttctcgtcgggagatgcaggaatt |
| | | tagccagattgacgccattgcccgcgtcattcgcggtaaagtcgccacttttcgcgaagatattgttgaaagcaacaaaggcaaagtgcgagagtcactgggtcagtatctac |
| | | aggtactaaacacgcaactcaccaatcatcgcgcattIctaaagaaaacagagccggaatttgacaaatgctgcgtcgcctttgctaacgccattgcagcctttgaacgccga |
| | | atcatcaataaccgccgtaaccgctggaacgactttttcaatgatctgatggaaaaaagcgacgacattgttgaagacgattttggtgataaagaggcgattgcccagcgtatt |
| | | agccagcagtttaaatcgcgtcgcgtcgaggtgaaaaaattaatgctccaggacactgaggagggcgttaaggccttacaggagcagatgattcaagcggtggctcgtttgt |
| | | tgcaagatattaagcacattgagttccagcagcatgtcgatttcgcccacggcggtgaattcgaatttggtcgcgagatcgcgctgggttatgaccttgggttaagggatttcg |
| | | gctcaatggcctttaaaatcggcagctacgccttaagcggcgccacagtcggtagcgccttcccggtgatcggtacggccattggtgccgtagcaggcgctttagtcggcgt |
| | | cgtcatgaccgttgtcggtttctttaccagcaaagcgtcgaaagttcgcaaagcgcaggggaaagtgcgcgacaagctagaaagcgccagagataaagcgctggacggt |
| | | attgatgatgaggtccgtaacctggttgcggctatcgagaatgaactgaaaagcagcctgctgcaaaaagtgaatgccatgcatacggcattgcagcagccgatcgccatttt |
| | | cgaacagcaaatcacgcaagtcacccatttaaaaaatcaactcgagaacatgccttatggaacaattcaaacagttcagtattgagaagcaggctgccattaactcgctgcta |
| | | cagctgcgcggcatgctggaaacgctgggcgaaatggagatcgatgtcaacgacgatctgcaaaaaatcgcgtcggccatcacagccgttgagtccgacgtgttgcgcat |
| | | tgccctgttgggggctttttcggacggtaaaaccagcgttatcgccgcctggctcggcaaaatcatggaagatatgaatatctcgatggacgaatcttctgaccgtctgagcat |
| | | ctataagccggaaggattacccggagaatgtgagatcgtagataccccggggctgtttggtgataaagaacgagaaatagacggcaaacaggtgatgtatgaagatctcac |
| | | caaacgttttatttccgaagcgcatctgcttttttacgttgtcgatgccactaatccgcttaaagagagtcacagcgccatcgcaaaatgggtgctacgcgatctgaataagctgt |
| | | catcgaccatcttcatcatcaacaaaatggatgaagtgactgatttaaccgatcaggcgctgtttgcagaacaggcggccatcaaaaaagagaacctaaagggcaagctac |
| | | agcgcgcggcaaacctgaatgcgctagagcttgaacagcttaatattgtttgcattgcttcaaatccaaacggtcgtggccttcccttctggttcaacaaacctgaacattacga |
| | | aagccgctcacgcatcaacgatctcaaaacagttgccgctgagattctgaaaaccaatgttcccgaagtgctgctggcgaaaactggcatggatgtggtgaaagatatcgtc |
| | | acccagcgtatcaccagcgcccagctgcatctcagcaaactcagcacgttcgttgcgaaaaatgatgaagatacttcgcgttttacatgcgatatccagcaaagccgtaacg |
| | | aggtcaaacgtctggctggcgaaatgtttgaagaacttagtttgctggaaaagcagctgatgagccagctacgcccgttggagctggatggcattcgcccctttatggacga |
| | | cgaactgggctataacgatgagggcgtcggctttaaattacacctgcgtattaagcatattgtggatcgcttttttgcgcaatcctccgccgtcacgcagcgactgtcggacga |
| | | tattactcgtcagcttaattccagcgagagcttcttaagcggagttggcgaaggggcatttaaatccctcggcggcgtgtttaaagggatttccaaaattagcccggagacgat |
| | | taaaaccacgatttttgctgcacgcgataccattgggcaattaacgggctatgtctacacctttaaaccgtgggaagcgaccaaactggctggcggcatcgctaagtgggctg |
| | | gtccggccggggccgcatttaccatcggctctgatctatgggatgcctataaagcgcatgaacgtgagcgagagctggaagaggcgaaaaatgagttgacccggatgatc |
| | | aaagatccgttcagcgatatctatagcgtcttgagttcagatgaaaagacgttcgctttctttgccccccagattcaagagatggaaaaagtcatttgcgatctgacagaaaaaa |
| | | gcgacaccattcggaagagccagcaaaagctaagcatactccagcagaagctcgagcagtttaaccgttcgagcgagcagcaagtgtcctgatacacaaacggcagccc |
| | | gcaggccacgtttagttataaatcaaactaaacgtggccaggtgacatgccccccgttgattaacacacgttatcgtcgggtggaaaggacaacctcctacgtccgcttcaca |
| | | gcggacactcaggtttaacagtccagtacgtttagcttacggataaatcattttatgatgatgtggagaatgggggat (SEQ ID NO: 407) |
| 44 | Dcm + | gacagcttccagggtatcgtggacgcgtcatgcaaagagatggggatgagggattttaatattctaccccttgtaccccatgccagtggtcgacctcataaatcattgattttaa |
| | HerA + | aagcctcacttagggcgctcgctgccaccgatgccccacgatgcctgacgatcttcaacgactccccgcaaaagtccctatgcctcggaaaagccgccaaccccaacaac |
| | Vsr | accacctaacaacaagaaacaggacctcgtgccgagcttgttagcgcgactgactagccgtccgaaagcaaaaacaccgcgagccaaacaaggcaatttcttgcccccct |
| | | aaggaaccacctgaggattgaacaccagcgcagcttactgtatataaaaacagttaaagtcctgttctcaggctgcatctggatcacacagccgccgttactcggaaacacg |
| | | gcggattagcgcgcacgctcaggccctccagccctaacggaatatgaatatccagaaaatcaaacacatatcagcctcacgcagcgcatagcgccctgccagaacacag |
| | | caggaagtcattgcgtttgcgttcctggcaatccatcattcacggttagggcccctataagacctgcagaagcagcgcgccatgggcagacccggcaaaagcccccaaac |
| | | gggtgtggagaagctttatggagaaggaaatcccccacgaaggattcacaggctctagtaaagagccgctccagacgctccttccctttaatatcgatgaacccgggcagg |
| | | agcccatgaaaatccaagatttccccccactccccgcctccgaacagccgttgatgtttgcagacttgtttgcaggctgtggtggcctgtccctcggtctctcactttcaggcat |
| | | gaacggcgtgtttgccatcgaacgcgacaagatggctttctcgaccctatccgccaacttgcttgaagggcggaaggtgccggctccgcagttttcatggccctcatggcta |
| | | ggcaagaaagcctgggcaatcgacgaggttctcgaaaagcacccgattgagctcagtcagctaaagggcaagatccatgtcttggcaggaggaccaccctgccaaggttt |
| | | cagctttgcaggaaaaaggaatgaatccgacccccgcaacaagctgttcgagaagtacgtcgaaatggtccaggccatccgaccatcggcccttgtcctggaaaatgtccc |
| | | tggaatgaaggtggcgcacgccacaaagaaatggaagcaactaggtatctcgatcaagccccagtcctactacgacaagctggtagagagtctggacaggatcggatac |
| | | cacgtccagggcaatatcgtcgactcctctcgcttcggggtacctcagaagcgcccacgcctgatagtaattgggctcagaaaggacctggcccagcacctcgaaggcgg |
| | | ggtagcccgagcctttgtgctgctagaggaagcccggctcaagcagctacaagagttcgaccttcccgaggccatccatgccgaggatgccatctcggatatggagatag |
| | | gtcacgcgggaacgaggccctgcaatgaccctgactcccctaggaaattcgaagagattgcctataccggccctcgaacggcgttccaaaggctcatgcatcgaggctgt |
| | | gatggcaccatcgatagcttgcgcctcgccaggcacaagccagagataaaggctaggttccaggcgatcatcgacgaccccaactgtgccaagggcgtacggatgaacg |
| | | ccgagatacgccaagcatatggactcaagaaacaccgcatctacccaatgcaggccagcgctccggctcccactatcacgacactgccggacgatgtcctccactacaag |
| | | gagcccaggatactgaccgttcgggagtctgctcgactgcagtcattcccggactggttccagttccgaggaaaattcaccactggcggtagccaacggacgaaggagtg |
| | | cccgcgctacacccaggtgggcaacgcggtaccaccttatttggcacgcgccgtcggcttggctatcaaggcaatgttggatgaggccgtgatgctcgccggccaacagg |
| | | cagagcgagaacaagaagagaaaatgatagccatcgcttgaacacataggagtcgaggggaatggatagctcccaactggaaggggcgcaatacccggccgcgcttgt |
| | | cgactgggccggccatcactcaggaggcgtaaaaaggctgctggataaaaatagcggccagcctaacaagcagctgctacggacgaaccttttgtcccgtctccaggcct |
| | | gggctaacaggcttcccaccgagacctcagctgtccccaggattgtcctgcttgtgggtggtcccgggaatgggaagacagaggcaatcgagtgcaccatccgctggctc |
| | | gacgagagcctcggctgcgatggccggttggtcgaggaactctcgaaagccttccatccctcaaccggctccgcagtcccccggctggccagggtagatgccggcagcc |
| | | ttgccaagctagatagcagactgagcctcgacattgtccaggatgcctctgctaccgccgggcatgagggaagcaccgcccccgtccttcttatagaggagcttgccaggc |
| | | tactggatggacctccgacccaagcctatctctgctgtgtcaatcgtggtgtcctcgatgatgccctgatccacgcaatagacaacaatctggaacaagcacgaactcttctcg |
| | | aggcggttacccgggctgtaagcctggcgtacaacgcgccttcatgctggcccctcgagggtttcccatccattgcagtctggccgatggatgccgagtcgctcttggtaaa |
| | | gccggacgacgagcccgtagcccctgccgagatactcctaggccaagccactgctcccgatatgtggccagcgaaaggggaatgcccagcaggcgacaaatgcccttt |
| | | ctgcgccagccaggccatcctcgcgcgggatgagaacagggcatccttgctgaagatattgcgctggtatgagctcgccagtggcaagcgttggagtttccgggacctgtt |
| | | ctccctcacctcgtacttgctagcaggccaccatcctgtagtccacgatccctcagggactccccaccagtccactccttgccaatgggctgcgaaccttgtcgacctcgacc |
| | | aaaaggccctaacggcgaaaaggcatggcaagcagtcgctaactgccattttccacctgtcgacttcgagctaccaacatgcgctcttccatcgctgggacaaggacgcag |
| | | ctacctcgctccgccgcgacctcaaggatcttggcctcgagaaggaactcgagatggaggaagggcgaaccctaatggggcttgtctatttcctttcggagcgcaaaagcc |
| | | actatctcccagcgaccatcgcccctctgctggaggggctggtcgaaacgctagatccagccttcgcaagcccagacggagaagttgcagtcagcagtcgaaacacaata |
| | | gtcctcggcgacttggatatgcgtttcagtcggtccctggccggaggtattgaattcgttcgtaagtaccaggtgctatcgccaaacgagctcgatttactccggcgcctatcc |
| | | gcatcagacgccatgctttcgttaccgagcatacggcgcaagaggccggtggccgccagccgagtccagcacgtcctccgtgatttcgcatgtcgcctagtacgcagaag |
| | | catatgcacccggacggccatcgtggcggacgctcccattctcgaggcattccagcaggtcgtcgaggacagcgacaagcaccatcacctcttcaaggtggtaaggcaa |
| | | gtaaaggaattgctgaacactgggaaggagttcgaggtgtcactaaccactacctttggccaaccactcccccctcgacaacgccaggcaacgctggtcgtcccgcagag |
| | | cccggtccggatgtccccccagaacaacaagggacgccctcacccaccgatttgctatctccatgtcggccaagggcaatcagtccagccagtcccactgacctacgacc |
| | | ttttcaaagccgtgaaggaactggaaagagggctctcacctgcatcccttccacgcacagtcgttgcactgctggacacgactaaggcccggctttccggcccgattgtccg |
| | | cgaccatgaactactcgatgatgcccggatccgcatcggcgcagatggcacggtggtcggccgctcgtggaatggttttgctgaaagccgggaggacgacgtatgagcct |
| | | tgcggatttcaagcagaccccgtggagcaaatcacatccgaactaccagaagtcggccctggcaatcagccctgcccctgagtatgcgagctcggaagtcctgcttgcctc |
| | | gctctaccgaaccataggcttcgcaacagccagcgagggcggcgtgccgcaggccgggcgagatctagacaagcgtatccagaaactccgcgagaaacgccaatccc |
| | | caccaacaggagcggtagtcggtgtagaggcttggaatactgtgcttcacgggatcctggagagcccgaagcttcccaaccagtcgtccaagcgtttcctccaggtaacgc |
| | | ccatcgtacccggggccgcactcttctccgggtctgcccgtctgagcagcaactcgtggcccgcaggcagcttgattcgccgcatggtctgcctgggatcgatggatgggg |
| | | agacggcgcaacgactttggcaacgcctcttcgctgcattgaacgtggacgacgaggacgatgtcttcgcacgctggcttgaccaagagacatcggcgtggaacccggg |
| | | agcaagcaactgggcactctcgccaatacccgcggacgagatggtcacgttggagacggcagatttcctggggatcccctttctccccgcccggcgatttaccaaggacct |
| | | acaggccatcatgcaggccaagggttcaatgacccgccggcagtggactagccttctcgaggcattgcttcgcctggcagccgcatcccacgtgacgtggctgtgcgacg |
| | | tccacgccaggacttggagctgcctgtgggccgcactaacggatggcattgctccttccagtgaactggaagcaagacgggcgctgttcccggaagccccgcagtacatg |
| | | acgtacgggggaaaagccctccaaggcatcaaggacaaggtgtctagctacctaaatgcccggctgggaatcaatgccctcctctggtctctggcgcagataggagctcc |
| | | ctattctggcaacctctcctcgagcgccggaattgctgcactttgccagcatattcgtcagcacaaggccgagcttactcgcctaggcacgcttgagacgattgccgatgtgc |
| | | gcgagcaagaagcccgtgcgcttctttgcaagaaaggcatcggctctaacctgctggagtttgcgcggcacgtccttgggcaacgccaggctgcagtcccattgctgagg |
| | | gggtacgaccagggatacatcctgaagaagaaaggcagcagcccgtccagcccatgggttgtctccctcggccccgtcgccgtgcttgccttggtccactgcgcccttgc |
| | | aggaatgggcggtccccgctcggtccaccggcttggacagcacctagaggcttatggcatggccgtggacaagcatgacattggcaggaacgacctgggccaccagttg |
| | | cgaatgctcggcctagtgctagatagccccgatgccgaaagtggcatgctgctactccccccgttccccataaaccaagccagccagggcccggaacatgaatagacttg |
| | | cacactggcttgccgccactgtccacgagaaagtcaggggctcgacacaagggttcggaggtaccagcctagaatatcggcttatcttccgcggcccacccctcgagcta |
| | | ctcgaaccggcctacgacgagctggcccgcaacggagggatccaggtgccaagcggggcagacggaggactggtgaccctgccggtactgctccagtatccagccgg |
| | | ccagctgcagggacccaggccacgcatcggagcatccggtaagtgtgacaacgaccacttgcttgatatacgcaacgaccctgccaaccctagctttattgccctggtccc |
| | | gccgggactgcacaacaacctctcgatcgagtcaaccaccgacgaattcggattgggggcagccaccagcacggggcatgcatccttcgaacaatggtgggaggatgg |
| | | ctttgtccagcaagcagtcaacgaggcgttgatcgctgccggcataacggacgcccagagggatgacgccaggggcctggtccgcgcaaccgcagcctcggtcgacga |
| | | ggtggatccagacaagggaggtcatcgcgcggcctggcgcctactctcgcgcatctactcgatagcaaacgtgaatcaagggttgcctgcaggaacagcgctatcactgg |
| | | catgtggtcttcccccaatgaaggagggaggaatttccgccaagactcagctttcggtcctgggaaaaatcgccgacgagcttgcggacggtttcaagactggcatcgagc |
| | | gcctggcacaaggcgtccaacaaggggttgcgcaagcgctgcgcgaactgctttcccatctccactcgaattgcgacgtacctacggccttcgagcgtgccacagcggctt |
| | | tctacctgcccagtgccgatattgaactggcgcctcctccatcctggtggaccacgctcaccaccgagcagtggacggaactacttgccgacgagcctgacgaggtcgtcg |
| | | gcgagctaacgatccggtgtaccaatagtttgatccctatggggaaaggcttgccggccgtagtacgggacaaagtcgagctattgatttccacaagcgaagagagccaac |
| | | caaaggagctcctgttgacaggcggatcctacggcaaggttccgacgtcattgccagcgggccctaatgggactaccagccacattgacctatttccctcctcccacaaagc |
| | | gccaatgagctacaaggtttccgcggacggctgcaagcctgcgagcgtccgggtcatctccctcgcgagctggaagcccggaatactcgttacctgcaggcttgcgacaa |
| | | agctctcgccaccgaggaagccccgcaagaactcagctgcgatggactgggaaacatccctgtcgctgccgggctccggtcgttatgagctccagctccaccttgctccg |
| | | ggggcgagcattggaaaggtagaaggcttgccggacgatgccaccgaattcgaggagcagcgggagacaatcgaaccacggcaagttggggaatacgagtatctaata |
| | | gaggtcgaggctgatggcaagtaccagctggacatcgcctttactgaagccggcgagcaagttccgaaggtctgccgggtatacctgacctgcgaagaggcaaaggagg |
| | | aaggttgcaggagcgaattcgagcggctcatcaagctcaaccgacggcatctcgagaagttcgataccaaggctgttgtccatcttgaccggaacgcacgctcctccagcc |
| | | tgcagtcgtgggtgctggaggatcagaacgtatccaattccttcaggccactggtgatcgcggacgactatgcgtcccggtgggcccctcctgactgggacgccccgcac |
| | | ggccctgtactctcgaacgggcgtttccttcatgacccccgccccgaggccacgagcttccaacctcccaagggcttcatcgaggctcggcaggggatcgcccggtacat |
| | | acgtggtagcgacgaccaatcggggctccttgagtcagcgccgcttggtgcctggctatccgaagaccctgggttccgctcccttgtcgaggactaccttggagcgttcatg |
| | | tcttggctggacgccgacccgggtatcgcctgctggatcgacaccattgccgtctgctccctggagccggatggtcgtaccctgggaaggatcccagacgccatcatccttt |
| | | cccccctgcacccattgcgcctcgcatggcactgcttcgcccagaaagtactccgtgacgaggccgagggcgaagccccgtgcccggcagcaagcatcctcgatccgg |
| | | actgcgtccccgatctactgaccatctcgctgcaggcaccgggaggagtggatcaggtcgacttcctttccgtcgaatgcagctccgactactggtccgtgctttggaacgg |
| | | atcccggctgggacaaatacccgatcgcgctcgccgggccccgttcgacagtagcttcgggctggcagttggagggatatcgagcgggttcagccccgcccaggtctca |
| | | cgagcactcgacgacgtcaccgacctcctggcagccaagcctatcgtcagcctggtagtgtccagcgcaggtggcaccacggatgcatgcaacgaagggttggccacct |
| | | ggtgcaccaagcgattcggcaacggggaccatgacaccccgcggcacggtgtcgggccaaggattgtggaggtattcgataccaggcaggctggccggcccgaccag |
| | | gcgacgatcgccaacctctccgaggacacaggcaaccacgtccgctggtatgacaagcaaccaactgggtccaagccagacctgggcatcattgcccaactagattcgg |
| | | cccaacccgaatccaaggaggtcggaatgctttcgccgatgggaaccggcggactgatcaggcaccgcgtcaggcgccaactccaagcctccttcctaagtgaatcccg |
| | | gcagggcctgcagatgccaccctccggcgaaccgttcgcagataaggtttccgcatgcatgctcatgatggaaaggctcagggacggcaaggtcggcctgcagttctccc |
| | | ctaatgtccatgcagtgtccagcatgctcgaggaaaacagcgctgggttcgtcgctgtatcgtcgtcagcaatcgaccccgcctgcttcctcggaggctggatacaagggac |
| | | gtatctatgggactacgacctcccctcgtactcgcatcgcgcaggcgacacaagcggctactacctgttatcacaggtcaagcaggctgatcgcgatgcgctacggcgagt |
| | | cttgaagccccttccgggatgcgaggatctggacgatgatcaggtcgagcaaatcctcctcgaggttgcgcggagggggattcctacggtgcgaggcctctccggggac |
| | | gatacgggggcgacgggcgaccttggcctgttcctcgctgtccggctcctacaggatcagttccgtgtgacaggcaacaaggaaagcctgctgccggtgcttgccggatc |
| | | accggaggactcgacgatagcaataatcatccccgtcgaccccttccggggttacctttccgatcttgcccgctcccttggcaaggagcgcaaggatacctccctgtcgcgt |
| | | cccgatctgctggtagtgggcgtgcgcgcatgcagcgacaagatccacctgcaccttacgcccatagaggtcaagtgcaggcaaggagtagtcttcggtgcaggcgaatc |
| | | aaccgaggcactctcccaagccaaggccctgtcgtcattgcttcgtgccatcgaggaacgtgcaggtagttctctggcatggcgccttgccttccagcacctgttgctctcaat |
| | | ggttggctttggcctgcgagtctacagccagcatcaggcagtaggtgggcatgccggccgctgggctagctaccatgaacgtatcgctgcagccatactcagcccaaccc |
| | | cgccgatcagcatcgatgagaaggggcggctgatcgtggtggacgcgtcgctccagagcagcccgcatgatcgcgatggcgacaagtacacagagaccattgtcatttc |
| | | cagccgagatgccggtcgtatcatcgttgggaatgacgcacagtccttctatgatggcgtacgtgcaaaggtcgacgactgggggctgctaccctgccaggcaagtgcgg |
| | | ccggcaccccaatcgtgcagcccgacatcactcccccggacgatgtccagacgggcgaccccatagtagtcccagcagaagatatccccggggcatccaccagtctggt |
| | | cgatcagacatctaccggcgtagcggaaccaggggcaagccctgcccccccaactgacgagccagggacagggatcattctctctgttggcaagactgtggatggtttcg |
| | | agcctcgatcactatccctgaacatatccgacacccggctcaaccagttgaacattggtgtcgttggcgacctcgggacaggcaagacccagttcctcaaatcgttaatcctg |
| | | cagatatccagggcccgcgaggccaaccgcggaatcacgccaaggttcctgatcttcgactacaagcgcgactacagcagccaggactttgtcgaggccacgggcgcc |
| | | aaggtggtgaaaccctatcgcctgcccctgaatctcttcgacaccacggggatgggggagtcctccgcaccatggctggacaggtttcgcttcttcgccgacgtactcgaca |
| | | aggtgtattccggcatcggccccgtgcagcgggacaaacttaagggtgcagtccgcagcgcctacgaggtggctggtgggcaaggccgccagccaacgatctacgatat |
| | | ccatgccgagtaccgagagctgctcgcagggaagtcggactcgccgatggctatcatcgacgacctagtggacatggaggtcttcgcgcgctcaggggaaacgaagcc |
| | | gttcgacgagttcctggatggagtcgtggtgatatccctcgattccatggggcaggacgacaggagcaagaacctgctcgtcgccatcatgctgaatatgttctacgagaac |
| | | atgctacgcacgccgaagcgccccttccttggcacgtccccacagctccgggccatcgactcgtacctattggtggacgaagcggacaacatcatgcgctatgagttcgac |
| | | gtgctccgcaagttgctactgcagggccgcgagttcgggacgggcgtcatccttgcctcgcagtacctgcggcatttcaaggcaggggcaaccgactaccgggaaccatt |
| | | gctgacctggttcatccacaaggtacccaacgcaacacccgcggagcttggagtactcggcttcacctcggacctggcagagctatcagagcgagtgaagacccttccca |
| | | accaccactgtctctacaagtcattcgacgtggctggagaggtcatacggggactgcctttcttcgaactcaccaaccaagcctgaccaacgcccggcctgcgaatacagg |
| | | ccgggcaaggaggctcctaatgacagacttcctttctcccgcagaacgctcggacaggatgtcacgtatccggggcaaggacacgcagcccgagctagcattacgcaag |
| | | gtccttcaccggctcggactccgataccgattgcatggcgcggggctactaggcaagccagatctcgtgttcccgcgatacaggaccgtggtattcgtgcatgggtgcttct |
| | | ggcataggcacaagggatgcaatatcgccacgatccctaagagcaacacacccttttggctggagaaattcgaaaagaatgtcgtacgtgacgcgcgagtagcaacagat |
| | | ttgcaggccttgggatggacggtacttgtcgtatgggagtgtgaactgacatctgccaaaaaagcccagaagactggcgaacgcctatatgaggttatccgtagtcgtagcc |
| | | acggaaagtatcggtaatcgactgaagcagccctgcggcctgtagtggtctactgatcccggacaccgatttaggcgaaaatcctcgccgtgagagaggtgtccg (SEQ |
| | | ID NO: 408) |
| 44 | Dcm + | cgaacggagcaggtagatccgcgctaactgacttgcccaatctggctgcattcgtccaacgctaggcggcttcgcaggaaaagcgaaacggagggagattctacgcgca |
| | HerA + | cctttgtgcagacctgaggctccaccagacctgagagcccggcacgattgactgatcataggagtaaggccaagaagcgacttgatgcgcttgtaaggtaaattctcagcg |
| | Vsr | aatcgaagtaatgacaccgaaacacgtgcggtcgacaaccgtgtaagattgctgataaaaagagcaggacgtcacaagaaatgaacttggaagtagtgccggcgagccg |
| | | gactttcatcgacctcttctcgggatgcggaggtttgtcgctgggactttgccaggctggatggaaaggactcttcgccatcgagaaggccacggatgcgttcgagactttcc |
| | | gggagaacttccttggtgagaactcccgctttgcctttgattggcccagctggttggagcagcgcgcacactccatcgatgacgttttggcactgcgcggtctacatttgtcga |
| | | aaatgcggggtgaagtcgacctcatcgcgggtggtccgccatgtcaaggattctcgttcgcgggcaagcgaaacgcgaaggatccccgtaaccagctctcccagcggta |
| | | cgtcgatttcgtcgagcgactccagccgaagtccctagttctggagaacgttcccggcatgaacgtcgcccataagtatgagcacgggaagagtcgcaagacttactacga |
| | | aaagcttctgcattcgctttcaatagccggctacgtggtgtcggggcgtgtcttggacgcggctgacttcggcgtcccgcagcgccgcactcgactaattgccgttgggattc |
| | | ggtcggatatcgcggataagcttgcatgcgcggctagctcgactcccgcagacgtgctcgagggcatcttcgatgcaatcaatcaggcaggcaagcgtcagctcgtccgat |
| | | atggccagggcgcccatgtcacggttcgggacgcgatctctgatctcgcgattgggccggccgatcacgagaacaccgaagactacgtgggaagcgagcgatgtgcag |
| | | gctacaggcaggtcaggtaccaggggccgaacacgccttaccagatcgccatggcttctggggtcaccccatccgaaatggacagcatgcgacttgcccgtcatcgtcct |
| | | gatgtagaaaagcgcttcaaggcgatccttgaaacttgcccgcgaggggtcaacttgagcgccgagttgagggcgcagcatagaatgctgaagcataggacggtgccga |
| | | tgcatcccgaaaagccggcgccaaccctgactaccctgccggatgacgtcctgcactaccgagacccgaggatcctgacggtccgggagtacgcccgaattcagtctttc |
| | | ccggactggttccgtttcaagggcaaatacaccacgggcggggcgtcccgtcgtcatgagtgcccgcggtacacgcaggttggcaatgcggtcccgccgctgctcgggc |
| | | aggccattggctcaggattaatggcgtgcctctctttgagttcaacgcgagtgataagggccagtgcgcccagtctcgcgatggccgagaaaaaggcttttgccgtatagca |
| | | attagtcagctgcaagaatcgaacaggtggatagacgatgacgaaataccccgatggattgcttgattggtcgggcaatcgggctggaggagtcaagaaactcttctacgg |
| | | cggcagcggccgccccgtcgggaaggtgatagagactcctctactcacccgtctctgggaatggtcggatagcgtcgtccagttcgagccgggcattccgcgggcggtgt |
| | | tgctgttgggagggccgggaaacggcaagacagaggcaattgagcagacgcttcgccgaattgactcaaggcttgcgctgagcggagcgctcatcgacaagcttgcgg |
| | | ctgtcttcgagtccaaggatggagtccccccaggacgccttgtggaggtggatcttggggcgctticaggggggcgctcgagcgggacaatctcgattgtccaagacgcct |
| | | cggaggggaatccgggctctcctgatcttccggcgcaattgctctgcaacgacctagcaggactcgtcgaagacaacgtgtcaaagcgcatctatttagcgtgcataaatcg |
| | | cggcgtcctagatgatgccctgatacttgcgacggaaagaggtgacacagaaattggtgctttgctgaagcaaatcatccggtcggtgtcgatggcggcccatggcgtctca |
| | | tgctggcctctgcagggatatccgggcatcgcagtctggccaatggatgtggagaccttggtcgcaggcgtccagggtcaaccttcacccgcggagcaggttcttcatattg |
| | | cggccaatgccgaccattggcctgatttcggggcatgcgaagcgggtcagtattgcccgttttgcacaagtcgcaggctcctttccggcgagccccatgcgggatctctcgc |
| | | caagctgctccgatggtatgagctggcgagcggaaagcgctggaacttcagggacctgttttcccttgtcgcccacctgttggctggaacccctagcaatgccgatgcgtcc |
| | | ggttattcgccctgcaaatgggcggcaaaacaactgaatccccccggcggcgacccgcgcaaggccgatgtactccgaaagcgcggagtctttcggttgctggcttccca |
| | | ataccaacacgcgctctttggcgactggccaatcgagcatgcgtcgggtctccgaagagacatcgccgacctagggcttggtgatttcccggcgcttgtggctatccagca |
| | | gttcctggcgctggataagcggcgggagtcgacggcaaccctccgtgcccagctctccggcatgtcatccgtattggatccagcaaaggcaagccccaccttcgaggtta |
| | | gggtaagcgctaatactgttattcgttacgaagacttggataggcggttcagcctgtccatccaaggaggcagagagtacctccaagaatatcagtgcctctcggagatcga |
| | | gatttcagcactcaaggtccttgaggaggccgacaataagttgtctgatcacttagtcaggcgatctcggccggcgacagcaattcgagtccaggcgcttctgagggccatc |
| | | gcgtgcaggctggcaaggaggtcgattggcgtcaggtgttgtgtcacaaaggatgccgacgtcctcgaggagttccaccgcgtcaccaatggcgattcgtcggcgctgca |
| | | gcaggcgatcaggcaggtcgaggcacttctcaacgtcaatcgccggttcgttgtttgtctcaacaacacctttggtgagccgctgcctcccccagagcggcgcgcgatgctt |
| | | accacggacattcagcgcgttaagccggtgcccgccttggagggtgttgagcggccgagatcgccgatgcccttcctgagggtcggcgcacaaggcaacgccaggccc |
| | | atagccctgaccttcgatctcttcaaggcgacgaaatcccttaggcgtggcatggtcgcgtcgtcacttccgaggtcggtggtcgcgcttctcgatacgacccgagctggtct |
| | | tgcgggagcgatcgtgcgagacgaagacgctctggaaggtgcggagatccggatcggaatcagggatgaggtcatagtgcggacctttggaagtttcgtcatccgccag |
| | | gagggtgcttgatgtccatgcaggagtttctcgcttcaccatggaagaaagaagcctcgcaccgagccttcaacgaatcctcttttggtatgaggtctgccccggagttcgca |
| | | actggcgaggtcgtcctgtcttcgctctaccgcgccgtcggctttgacggggtttccgaggagaaagtgccctcgcttggcaatgatttcaggaaggcgctggacaaggaa |
| | | cgcagaaagcagaacgcagctggtggtctgagcccagaagcctggcgcacggtcgtggatcgtgtcgtgcaaagtcctaaggttgcgcagcaatcctccaagcgattcct |
| | | atcgctgtccccggtcgttcccgacgcggccatctactcgggcgccgcgcgccttggaggaaactcctggaacccggggcggctgatcaagcaaatggtcggaatcggg |
| | | tcggagaccatggagggcgcggaaacgctttggggcgaactctacgatgctttgtccgtgacggaagcggatgatgtctgggcaagatggctccaaacagaatttagtccc |
| | | aggcgcccagagcaaatagcgtgggccccaagaccgatggatcaaccagatttgcttccgcaatccgatagacggggagtttcctatcccgctcggcagttcgtggtgga |
| | | cctgcgaggaatcttggatgcgaagtccgccatgacgcggcggcagtggatcacactgctcgaggcgctacttcgaattggatcggtcagccatgtgctgtggctgtgcga |
| | | cgtcaatgaccgcttgtggcgtgcgatgcgtgcggcgctcgagggcgaggcgagtggcgtgcccgccgatgccgccgccataagaaccgacattctggccgtcaggcg |
| | | gcggacgctctcgttcgggaatcccgctgtcccagcgattcgggacctggcctctcgatacctatccgcacgcctgggaatcaactgtgtcctttggacgctggacgaactt |
| | | ggcgtgggctcaagtcgactttgttcgtccgaagaaatccttgacttcatcaagagcgttcaggccaacgcaggggggctcaaggcccgtggcgtcatggatgccttccatt |
| | | ccctgcaagacaaggaagtcaggaccattggctgtaagaaaggagtcggagcaaaccttctggaattcagccagtacacgcttggacagaggcagacgatggaccagg |
| | | cactccgcgggtacgaccagagctatttcctcaggaagaacggggatgccaggaacgcgccatgggttctatctctagggcccgctgccgtacttgcgatggtccactcgt |
| | | gcctacatgcggtggatggaccgcgatcgatacaaaggctttcatcccatctcgggagctacggcatcgagtttgatctccacggcgtcaacgatagcgtccttggaaagca |
| | | actccgaatgctcggactcgtactggatagcccggatgccgagagcggtatgctccttgtgcccccgttcgtagcctgaggaaggaggcaatgatgagcacgctagccaa |
| | | gggaattgcaagctgggtcgaaaaagccatggcgcgtgagatcgcgacgctggtggccgggaatatggagtgtcgcgcagtcttctgcggcccgccaaagcacatcctg |
| | | aatcaagtatttgggcatcttatccacggtcgatcgctgatcgaagcgacaagggccgatggtcaggcggttcagtatcccgtgatccttcaggtcgaccgcctccctacag |
| | | ggtttcccatcggctccgccacacagtcgggatgccttcagttccatggactcgctgccgtcaggaacgacaggaatggtgttttcctagttcttgtcgagcccggtgctcaa |
| | | gcgagcgatacgcatgaatcaactcgaacttcgcttggactcgagccatcggtaaacgagggcggtgcctcgatcattgcctggtggtctgatccattcattcagtcgcttgtt |
| | | gattctgccctctcagaactctccggtcgcgacgccgcggctgccaaggatctactaaaggaggcgatgatcgccgccgacgcggcagatcagcacgaagtagcgaga |
| | | gttggagcctggcgcgtcatcgaacggttgtgggagctaaaagaacgcggcttgtctcttgaccaactcgttagcttggccgccggattcccgccctctagcgacggaagta |
| | | ttgaaccgagatccaagaccgccatcctttcagccatcgtggacaggatcgaagccgagaacttcggtggcttactgtcgtcccttctgcaaaaagccagggacgatatcga |
| | | aaaagaacacatcaccgcgtgcctctcgaatatgaggggcaggtgcgatgtggttactgcggttcggcgatgtgcgccatatgcgtacatgccttcggacgccatcgctgg |
| | | cgaagtctggtggaagtcgctcactgtcgagcgctgggaagagttgctcgatgatggcgctctacccgatgcgggcggcgacatcattattcagtgtgccaatccgatgattt |
| | | cgcaccttaagggcatggttcccgtcgtcaagggatccgtgcaacttaggatcgaggttccagagaagtacgtgggcaggcggttggaggttatccgcgaggtcccgggt |
| | | gcgaaggcggcgacgaaggtttggacagttgacgcggaacgcatgatccacgtcgaggacgacgagatccccccccacaagagtccgatgaagtactcggcaagcctc |
| | | gaaggatcagccggaaagaaggcgagcgttcgaattgtctcaatggatggctggctccctggggtggttgcctctgcgacgacggcgacaaaaggttccctcccgaaac |
| | | gctcaaaagcagcgaagttagaggcgtcgctgtctctctccgggcaggggaggcactaccttgacatctacttaaggccgggcgtcgagctcgcgtcaatgctcgccacc |
| | | ggtagtgacgaggaaggaaatccagacccgtccatcacggcgccaatcggcatggtcgcggagggcgagttcggggtcgaaatcgaaatcgaaggggaatgcttcttc |
| | | gacatcacgctcagggttccggaggttgcggatgatcaggtcatccggatcgaattgtcggcggagcaatcaagcccggaagagtgctcaagccacttcgaattgcagctc |
| | | cttaagaactctagcggtcggaagcccagcgcggtccacgttaatgctcagctaagaagtgcgcagcttcaaggttggatgctggagcaggggcgcgctggtcgctcctat |
| | | tatcccttcgttatggccgcggactatgccgccgactggcacaggcgggactggactggcgcagatgacacgatcttctcgaaggctagcttcctgtgcgatccccggccc |
| | | tcgccggaagaaatggcgccgccgcaggctttcatagatgccagagccgcactggccgccaggatcaggggtggtgacggaaatggcttggtcgaaggtgtgccgctc |
| | | ggtgagtggatggcaacggatcccgatttcgccggggaaatagacgtctacttgaaatcctacatgcactggcttgcgagcgatccagatggggcggtttggtgtgacgta |
| | | gggttggtcgcgcggctcgagcctaacggacttaccttggtgcaagagccggatgcggtgatagttagcccgatgcatccggtaagacttgcttggcactgtgtggcccag |
| | | cgagccatgttccttgccgcacgaaagagaccttgtccagccgccagcatcctcgatccggattgtgtgcccgatgcgatcactctcccactgagaaacgccatgggtggc |
| | | aagaccaacgccacttttttctcggtcgaatgcagttcggactactggtcgattctttggaacgcggggcgcttggaagccctttcttcacatggggcgacagccccgcttgac |
| | | cgggagtttggcctactcgtcggcggaatctccggtgggtttagtgtttcgcaggtgcacaaagcgctcgaggacatctgttcgatgctggtggcgaagccggtcgtcggc |
| | | gtcctggtgtccagtaccgcgagccagaacaatgcgtgcaatgaaggtctgctttcctggggcaggaagtacttcggcggcggggatagggcggcaggcttggacgcct |
| | | gggtcggggccagcgaggtcaggatctacgacgacagaccggaagatgcccggcctgatgatgcggagatttcaaatctggccgaggatacggcgaacgccgtgcact |
| | | ggtattccggcacggtggccggcgaggctcccgatctagcgatcatcgcccagcttgagacctccaatcccggtgcactcccaaccaaactaaattctccgttgggcttcgg |
| | | tgggctcgtgaggacccgaattcgggagccttccagcatggcggggggtcaactgctccgtgagtcgcgcatgtctggtcccgcggcgcccactggcgacgggctggc |
| | | cgacgctgtagcaagtgccatctcgtcgctcgagaacatctcggagcaacgccttggttacgtattcgcccctagcattcatgtgatcaagggggcgctggagagcgcgga |
| | | atttgccgcagtttcctcttcgagcgttgacccggcctgctttctcggaagttggttggagggcacctatctttgggactacgagctcccgtcgtactcaggtcgtgccggaga |
| | | cagcaatggctactacttgttgtcacggatcaaggatctcgacctcgaaaccctgagaagcgtggtcaagaggttccccggttgcgaggagatgccggaagccgtgcttgc |
| | | tggaatagtcgaggaggtcgcacggcgtggtattccaaccgtcaggggcctcgccgcaggtgattctggcgcgacgggtgatttggggctactcgtggccacgaggctg |
| | | cttcaggatagcttccgggcggccgaatcaggcgctggtctcctgacgccttggcgcagggagggagacatcgaagagcttgctctcgtcattccggtggatccattccag |
| | | ggctatcttgacgatctcgcgaaggcgctaaagcgccctacgctccaccgcccagacctattggtcgcgacggtgcgaatcagtgacctgggagttcaggtccgactgact |
| | | cccatcgaggtcaagaaccggggtgctggagcggcgatgccgcaatccgatcgagaagccgcgcttgcccaggcacgctcgctggcatccctgctagatgcaatgctg |
| | | gcaacgtattctgaggatcaagagatggttctctggcggattgcgcaccagaacctcttgacctcgatgatcgggtacgcattccgtgtttacagccaacgtctggcagccca |
| | | aggcaagtcgggagactggtcgcgcctgcacgcacgagtcatggaagcaatcctgagctcccaggccgatgtgcgggtggattcgagaggccgcctgatcgtgatcgat |
| | | ggctctagccaaagtggtccgagggatacagatggagatggtttccacgagactatcgagctctcgcacaaggatgctgcgcttttcatccgtggcgagcacgatgcgctct |
| | | gcacggccatgaagcagaagctaggtggctgggaaatgttccctgaagggagggatgccggactctccaatcaatcgccgcccgtggcccatgagactgcgcccttggt |
| | | ggatggcggcgttgaggtgccgtcccttcacgcgctccaagcaacggcggggcccgagggcagctcgctgccgtcttcgggagtcgaagccatgggcgcgtcgcagc |
| | | cggcctccccgggagccatcgacgtggatggcggcatggcccagtccgggctgatcattcgggtcggtgaaacgatcgatgggtttgagagccaaattcggcggctgaa |
| | | tcttggcaacacggccctgaaccaaatgaacatgggagtcgtcggcgatctggggaccggtaagacgcagctgctccagtctctggtttaccagatagccaaggggaaag |
| | | atggaaatagaggtattgagccgagcgtcctcatcttcgactacaaaaaggattactcttcgaaggagttcgttgatgcggtagctgccagggtcattagccctcatcaccttc |
| | | ctctcaacttgttcgatgtttcaactgcatcgcagtccatcaatccaaagctcgagcgctacaagttcttctccgacgttctggacaagatctattcagggatcgggccgaagca |
| | | gcgagaccgccttaagaactccgtcaaggacgcatatgtgcaagccgccgaagggcagtatccaacgatttacgacgtccatcgaaattacgtagaagcacttgatggag |
| | | gcgcggactccctgtcgggaatcctaggcgacctcgtagacatggagctcttcacgccggatccaagtgtcgttgtttcgtcggccgaattcctgcgcggagtggtcgtgat |
| | | atcgctaaatgaacttggttccgatgaccggaccaagaacatgctcgtggccatcatgctcaacgtcttctacgagcacatgctgcggatacagaagcggcctttccttgggg |
| | | agaaccgcaatatgcgtgttgtcgactccatgctgctcgttgacgaggccgacaacatcatgaagtatgaattcgacgtcctgcgtcgggtcctcctgcagggacgtgagttt |
| | | ggcgtcggggtgatcctcgcttcgcagtacttgagtcacttcaaggcaggtgcgacggactaccgggagcctttgctttcctggttcatacacaaggtcccgaacgttcgtcc |
| | | gcaggagctttcggcgcttggctttagtgatgcggtgggattgccgcaattggcggagcgtatccgtagccttggcgtccatgaatgtctctacaagactcatgacgtgcaag |
| | | gtgagttcgtccgcggcgcgcccttctacagacggggtgagtgggccaaggaatgacttttcgtcgtgtcgatttatcgcctagttacgcttttggtcttaagttgcgttcctaag |
| | | agaggtgggctgtgtccgacaatgcgtattacgtttatgcgctgaaagatccacggatggcgcccgcccagccgttctacataggtaaaggaaccgggacgcgctcccatg |
| | | accatcttgtaaggccagacgattcaaagaagggaagcaagatctccgagatcatggcctcagggcgtcaggtgctggtaacccggctcgtggacgggctcacagaaga |
| | | gcaagcgttgagaattgaggccgagcttattgccgcttttggcaccctcgatactggggggatgctcctgaattccgttctgccaagcgggttggtaaacaagagccgtagct |
| | | cgctggttgtcccgtctggcgtaagggagaaggctcagattggtctggcccttctaaaggacgccgttctggagctggccaaggcgaatccgactggtatctcgaactccg |
| | | atgctgcgagcatgctcggcctgcgtagcgactacggcggaggatcgaaggactatctgtcgtacagcctcctcgggctgctcatgcgggagggaaagctcgctcgggtt |
| | | gccggcactaagcggcacgttgctcaagtgagctagctgtggggttccggatcgggctggcccgctcggcgctgcgctacgaagctcgcttgcctgccaaggatgctgc |
| | | ggtcatcgaacgcatgaagcactacgccgcgctgtatccgcggttttgctatcgccggatccatatctatctggagcgcgagggcttccatctcggctgggaccggatgtt |
| | | (SEQ ID NO: 409) |
| 45 | RecQ | atttgcctgagacttatttcccgtggcgcttagctagctaagagtgggcatcgtgagcaccattgatgatatgaaatgacggtatagcaatttaaccgtctggatttcaccagaa |
| | | attagtgattcaataggaaattaaatacgttttatatttcaatgtgtatcaaaatcattcctgaaatttcctggtgctatatttgatgaaaacggataaacattctgttgattttaataaaa |
| | | ttctgtctttcgatttagagcttacgcgtgatgaaaagttaaggcatatgggggccgtgctggcggaacgcacgttgagtttgaagataaatcaggatgaagcgattcatcaatt |
| | | ggatgaaatggcaggcgatgcagatttaatcctcggtcataacatactggatcatgatttaccctggattgccaaacaacgcgtacgtgctcaaatattattagataaaccaatc |
| | | attgataccctttatttatcaccgctagcttttcccgcaaatccataccatcggctgattaaagactataaactggtaagagatagcattaacgatccagtgaatgacgctaaatta |
| | | tcgcttcaggtattcaccgagcaaatatgtgcgctgcaagaaaagccgctggctcagttgcagctatatcagtatctttttgagcacggcgttgccagccatttcagtacacgtg |
| | | ggatggccagcattttttccgcactgacgggtcaggcgtccatatccgccgtagttttacctacgctagttaaatcggttgctcagaataaagcatgccctaaccagcttaatcg |
| | | ggttattggcgatgctcttaaacagcctttgcgcttactaccattggcttttgcctgtgcctggctccccgtatcgggagggaattctgttttaccgccctggatatggcgccgtttt |
| | | cccgtcaccgctgatatcatccgcgaactgcgtgagcaaaaatgccagtctgaaacttgccgctactgctgtgaaaaccatgatgctcgtcggcatttacagaaaattttcgag |
| | | ctgaacgattttcgtaaacttcctgatggctcgccgttacagcgcaatatcgttgagtacggattagctagtcgttcactgcttgggatattaccgactagcggagggaagtcttt |
| | | atgttatcaacttcctgcgattgtcaggaatctgcgaaatggttctttaaccattgttatttcgcctttacaagcgctgatgaaagatcaagtggataatttacgtcataaggcaggt |
| | | attaaaggcgttgaggccatttcagggatgctaactttacctgagcgcggcgctattcttgagcaggtccgtaagggggatattgcgattctttacctctctcctgagcaattac |
| | | gtaaccgcgcggtaaaacaagctatcaagcaacgtcagattagtggatgggtttttgatgaggctcactgtttatcaaagtggggccatgattttcgtcctgactatctgtattgt |
| | | ggcaaggttattgaatctttggcgcaggagcagtctgtgcagattcctccggtattttgctataccgcaacggcgaagttggatgtgattaatgatatttgtcggtattttgacaaa |
| | | aaattatcgcacccattagctcgtttttcagggggagtagaaagaattaatcttcactatgaaatcattgcaagtaatggcttgagcaaaattagtcagattttgaatttgctcgata |
| | | aatttttttctaatgatgatgaaggtgcatgcattatctattgcgcgacccgccgttcggtagatgaaatcagcgatgtgttgacccaacagcaacctttaccggttgctcgttttta |
| | | tgcccggcttgaaaatagtgaaaagaaagaaatccttgaagggtttattgctaaccgttatcgagttatttgtgctactaatgcctttggcatgggaatagacaaagaaaatgtac |
| | | gtttagtaatacatgcggagatccccggttctctggaaaattatctccaggaggcagggcgtgctgggcgggatacgctggacgcgcattgtgtgctattatttgatgagcag |
| | | gacattgaaaaacagtttcgccttcaggctattagtgaagtaagctttaaagatatttatgcaatatttaagggaatcaaaaagaaagttaatgaaaataatgaagtcgttgccac |
| | | aagtattgagctaattaatcatcctatggttaaaaccagtttctctatcgatgataacaatgcggatactaaagttaaaacggggatagcgtggctggaacgtgttggttatgtgg |
| | | agcgacttgataatataactcaggtttttcagggaaaagtggcctttccttctctggaagaagcgcaaagtaagatggcagcgctgcacttgaatcctgcggcgatggttctct |
| | | ggaatgctgttttacaggcgctattaaatgctaatgacgatgacggacttagtgccgacagcattgctgatgaggttgcccaatttcttccgcataaagaaaataatacgtcagg |
| | | aattgaagcaaaagatgttatgcgcgtattgacacagatggctgatgttggcctggtcaccaggggaatgctgctgaccgtacgtatgcgccccaaagggaaagataatgc |
| | | gaggatcacaactgagttaattcacaatattgaaatcgccatgttagggctgctgcgcgaagctcatcctgatattgaactggggatgccatggcctctccagattgcggttat |
| | | gaatcaagagattattcagcaaggctatgatagaagtaataccacgttactacaaaatatattatttagctggtctcaggatgctcgagcaaacggtcataaagggcttattgatt |
| | | ttcgttatggtacaaggaacagctaccagattattatgtatcgtgactgggcatatatcgaaagagccattttacaacgtcatcgtgtgacaagctccgtactgaattttatttatca |
| | | attggcattggatagtgatgaaagcagtatcaaaaaagtgatgctttctttctcactggaacaggttatcgattatttaagaaaagatgttgatattattccaatgatccaacagag |
| | | acaggggggggatgagcagcagtggctgatggctggtgcagaacgtgctctactttatcttcatgaacaacatgccattgtgctgcaaaatgggctggctgttttccggaca |
| | | gcgatgagcttgaaattgcaggctgaaaaatcgcaacggtatgtcaaagctgattatgaaccactggctctccattatcagcaaaagacgcttcagatccatgtgatgaatga |
| | | atacgccaggcttggtcttgaaaaacctaactatgcccaacggctcgtacaggattactttgctatggatgccgagtcatttgttccactttattttaaagggcggcgaaaaattct |
| | | cgatctggcaaccagcgaaagctcatggaaacgcattgttgaaaatttgcataatcccgatcaggagcaaattgtgcaggcgagccttgaacaaaatacgttagttcttgccg |
| | | gaccaggctcagggaaaagtaaagttattatccatcgatgcgcctatcttttacgcgtgaagcaggtcgacccgcgtaaaatcctgttgctctgctataaccgtaacgcagcg |
| | | atttccttaagacgcagattgaagtcgttgcttggtaaagatggcgccagcataatggtacaaaccttccacggattagcattgagccttacgggataccagattgagcggaa |
| | | agataatgacgaaatcgattttgataacctgctctggaaagcaatagctttactcaaaggcgatgaaacgcagctcgggttagaagttgaagaacaacgtgaatacctcctcg |
| | | gcgggcttgagtatttactagtggatgaatatcaggatattgatgagccacagtatcagctgattgccgcgctggcaggtaaaaatgaaagtgaagatgatgctcgtcttaatct |
| | | catggcggtgggtgatgacgatcaatctatttatggtttccgtgatgccagcgtgcgatttattcgtttgtttgaaagcgattactccgcccgtactcattttttaacgtggaattacc |
| | | gctctacggccaatattattgcatgttcaaattatcttatcagtcataatcaggggagaatgaaatgcgagcatccgatcgtaatcgatcgcgctcgccagatgcttccgccagg |
| | | cggagagtggagcgcacttgaaccttcggaaggcaaagttgttatccagcattgtaccggcgcggctcagcaggcggcagaagtcgtgcgccaaattcagtatattcaacg |
| | | gctgcagccggaatgccctcttgagaaaattgcggttattgcacgcaatgggctcgacaaaaaggagcttatttgggtccgttcagcccttgcggatgcaggtattccttgcc |
| | | gctttgcgctggagaaagattatggtttccccattcgccactgtcgggagatcgccaattatctgctatggctacgagaaagagcgctcgagtcgctgacgccagcagagct |
| | | gtgtcagcaactaccggggcgagaccaggcgaaccgttggcacgatattatttatgaattaattgagcaatgggagctaagccagggaggcgagccattacctgccgctta |
| | | ttttgaacatttcatactggaatatttacatgcccagcacagccaggttcgctttggcctgggggttttgctgagcaccgtacatggcgtaaaaggtgaagagtttgagcatgtc |
| | | attatattagatggaggttggcgtagttcgcactctctgcaacctgaaaataacgaagaagaacgaaggctcttttatgttggcatgacgcgagcgatatcccgacttgttattat |
| | | gcatgatgatcgtgcgccaaatccctatatcgaacagttagatccagcggtcatcagccatactgctgcacaagccgttgcgcctgggatcttacgtcgtttctcgatcatcgg |
| | | attgcgccagctctatatcagttttgcaggtggacatccggctggtcatcccattcattcgttacttaccgatatgcaggttggggatagcgtccaactggtctctgtcgggaata |
| | | ccatcaaggtgaatgctaatcaatcggcaattgcgcagctttcaagtgccggaaagagccagtggcaattttctctttccgggatccgcaaaattgaagtgcttgccatgctac |
| | | agcgcagcaaaacactaacagcagaggattatcaagttgcggtgaaagtggacaattggtatgtaccgatattattggttgaaacccgtgaagaagccgcttatgacaatatt |
| | | acttgaagcagaatac (SEQ ID NO: 410) |
| 46 | Histidine | aactcacccgctctgaacgagccccttgaaacacaagacaccgtttttcccttaccataagggataggcaaacgactgtgtttatgactaccagcagagacaaaaccatcga |
| | kinase + | agtgctcggccacccatttgcgcctctaggttgctacgagactgcagaggatccatgtagcagattacctcggccatgaagctgctaacggaagcgaagccatagaccgta |
| | phospho- | ggcgatacacgtacgtatggctttccggaagggcgatcctagtcaactgtctgatgtccgccaaatctttctcaatactggtcattcaccttttccttgaccggctgtcaggccca |
| | ribosyl- | acgtgcattcagatcgtcgcctaaatttgttgcatcacgtagagtctgccgcgtgctcgcccctatgccagactagtctgatgtggcggatgagataggtcacgacggtggtg |
| | trans- | gctcggtagagtcggcatcgccgagtcaacgatggaacgtaaggggcgtgaatgcaaatcagccgtaagctcaacctttatgagatcgaggatctctaccagtcgcttggt |
| | ferase | acggattccaatctcaggcttcctatcagcatgagccacggcggggggttgggcgtggatgcttcgctggcccagttcatcgtcacctgggcacgtgcttgcgaaaaaacc |
| | | gtccttcacctatatgcccccgctggcgacgacgccatgacgcaaatcacgcagttggcgcagagtgcttctgggttcttcgcgctgatcatgtgcagtgaagtccacgctca |
| | | gaatcatcaactgatcgatcggcgggaagcgcttctggcgatcaggccccttgtcgatgcgatgttcgcaggcgaccttcgtaacacctccaacatccgaggcgcccgtcc |
| | | aacggccatcaatctgttctgcgtgaacaacgcaaagcgtgagttcatcaagccgttttacttcgatcacgccgtgccgaaagtccagccgagatcttggttctcgactctcttg |
| | | gagacgtcatcgaagctgatgaatgctcgcagtggacaaggggcactgcttaggtcaggtctcccggcattgggcagcgtgctttgggagttgatctccaacgctgaccag |
| | | cacgctgtcactgatgtaggcgggaacaagtacaagaaggcgctgcgtggcacctccatcaaactcaaccgaatgagtcgtcaggatgcgctgatgtattcagaccaaga |
| | | gccggagttggcgcgctttatcctgaagcatttcctgagagctgaggtactggacttcctggaagtctcggtcatcgacagcggtcctggactggcacggcggtggctgac |
| | | ggcgaaggaggggcggccagtagaaagcctggaggagctgagtcttgaggctgagcttgaggccacgctcgattgcttcaaaaagcacattacatccaagccgcagtct |
| | | ccgaactcgggtatggggctgcataacgctgttcaagcactcaacaagctcaaggcgttcgtacgcgttcggacgggtcggctttcactgcatcaggcttttcagggaagtg |
| | | atgagattatggagttcgatccgtcgattcgatacggtggccgtgtgttggccgctgtggaaggcactgtcttcaccatctgcattccggtgagctgacatgttcgatctcatgg |
| | | attttgaagtcgagttgcgtcagtcaggtaagccggttcatgtggtggttttcttcactggccctgatctcctcacagacacgcaagcggctcacgctctacagcaccaattgtc |
| | | gggttacgtcatgcctgacctagtggtgtttctgatgcctggttacaccttggatgaattccgagcacaccaggcaaatgctacatcgcccctgatggcggagctaagccgta |
| | | aaggcccaggctcgcctcgcacctacgcgagtgcgttctatgacgtgaatggtgccattaccgagtacgtcaatatctctggccctgaggagcagttcgaggaactcatcaa |
| | | gcacaactctaacgctatcgcgaggactggcctgacccacctcgtcgaacgctccaacgtgctgaagaaggcgcctgcaggcttcttctactcaaagccctcttctcgggct |
| | | tcgaactatttcattcgggcggaagacctgctctctgagaccttgcatgcccactacctggcgtttgcatgcctatctctcatcagtaaggcaacggaagatgggatggggac |
| | | gcccgataccctgtatctggacacaatcgcattgctgcctctggcgctgtccatgcaggtgtacctcatgcgatttgagcagccgggctttgcgaatatccggtcattccattcg |
| | | cacgaaggcctaatcaagggtgggcctttgcccaaggcagtttccgccctgtgtctcatttccgcatcgacccagtgcggcctcgcgcagcaatgggtgaaggtaaacagt |
| | | gctccgccgacgcgcgtggccaccattctttcatttgagcgctcatcggactcctgctccgtcttgcacacactgaagcagcccgaagactttgaaatgttgggggagggtg |
| | | aagcgagcgggattcgtctaattcggatccatggcgagcggttcgttgctgagcacagtgaaaccaagctgctgaacatcggcactgatcatgcgccgcccctgctgcaat |
| | | ccaagttctactcgttcatgggggccaacctgttcagctgcttcacccatgaccggccaggactgaggcctcggacagtgcatgtcgataaagataacctggtggctgccag |
| | | cgatttcggtgaatggttcgacagggtactgcttgaggaagctgtcgcgtcgacccgttggatcatccacgatgacgacgctgccagtgcggccctggccgatcgagcgat |
| | | cgcttacttagggatgtgtggcgtcaaggtcggtaacaaggtctccttcgatgacttcgatgccaacacgaattttgacgggtctgtcatcgtcattgccgctgctgccgaacgt |
| | | ggctcacgcctgcagagtgtgagccgacgcctgcgtaccgctcagcaatcgggtaccaggctttacattacgggggcactcttcgggcgcagctatcaactgatgaaggat |
| | | ctgcagagcaacctgacgcaacctgccaaggatcacagccggtatgttttcaagacgtacatggagatcccggcagcggagcttgcctgcacgagtcattgggccgaaga |
| | | gcagcggctgctcatctccttgcattcatttgcggaaactttctcgccagcgattacgcagcgcatggaagtatttgatcgcgcctctactggggggcttggtctgaacccatttt |
| | | ggccgagcagtcacaccgggcagccgatgacacttagccgaggctttgcgtttgtcgacggtacgaaggatgtgaggggcgcgacgtcaacggatatttacctaaccatc |
| | | ttgtggattctgcagaatgcccggtacagcggtaaggtgcagaacgccaagcggcttgagtccggtgagcttcagcaggtgctcctatcgccggatgtgttctcgcgcttcg |
| | | acgatggcgttatccaggccgcattcttgcgcgcagcggtgccggcggagcttgactacagggctcatgaaacccacagcctggccatatcggacatcattcagcgcatc |
| | | gccgcagggtacggacatgaacgtggtgaagccgccatggagtttgtcatggccttggctatcgggaagatacgactgcacaaggatgtcgataaccggctgcggagtaa |
| | | cttgatcaatatcttgacgccgcacgttcaggagatccgttatctgctggatccgaattacgaatcaccgttgtgatcaatttccgctaacccgttgcatgcgaggtatccagtta |
| | | ccggcaactcagctcatggctgagctgaaccctggttgctcttctagtttcgatggcttgccgattgccgggatcacccacctgcgtcggttctgcgacgaaggtctaagggc |
| | | agggtggtggcacctggcttgctcattccgtttgacctcgccaccat (SEQ ID NO: 411) |
| 47 | PH- | cgctcagtccggttggtggttttggttggtttggcgattgctcagatcgcacaatccgggctgagttccctttcagtgatctactattccgcgcagctatttagtggatataatcac |
| | TerB- | gctttgaaaaaaaaacgggtcaattactcttcgccccacagcaacgaataaggagaaatttgtgagtaacgtcaacactttccttaaggaaaatttatcttcagtaagtaagaat |
| | DUF726 + | gtttttgtggctcctggcatccctgaaaaaaaactgaataatgtcgctaaagcatttaatgttgtggataacttgaatactgtgctagccatttatgacaatacggtatttggtagcg |
| | TM | caaaagatggcatcgtttttaccggtgaaaaactggtcataaaagaagcttttgaaagtccttatgacttgttctacagcaatattgaagcagtagaatatatagaagatgtcacg |
| | | gtaaatgataaaggcaaggagaagcgaacagagtctgtttccctcaaactaaaaaatggcgaggtaaaacgaatcaaaggcttgatggagtgcaactataagaagttgagc |
| | | gacattcttaagcataccatcagtgactttgatgagttcaaagaagaagatcagctcatcactcttgccgaaatgtcagaagctctcaaagtggcttatgtcaaaatcattgtgaa |
| | | catggcgttctcagatgatggtcaggttgataaaaaagaatttgccgaaattctcttgttgatgacccgacttgagttaacgactgaatcccggtttacactgcgtagttatgtcg |
| | | gttcagaatccagtctgataccggttgaagaattaattgcgatcattgaccgggaatgtgtcccaagccataacaaatcaataaaagtctctcttgttaaagacctgattagcattt |
| | | tcatgagtgttaatgaaggtgaatataaaaaattcccgtttcttcagcaagtgcaacctttgctgggcgtaactgacgaagaaatagaactcgcagtaatggctattcagcaaga |
| | | ttttaagatgttacgggaagatttttccgatgatgcgctgaaacgcagtatgaaagaacttacggcaaaagcaggtgcggtaggcgtgccactcgctgctgtctatctctctgg |
| | | ctctgtcatcggtatgtccgcagcgggcatcacttctgggcttgcaacacttggacttggtggcgtgctgggtttttcaagtatggcaacaggtatcggtgttgcggtgttattag |
| | | gtgtaggtgcctataaagggattcgtcatcttacgggtgccaatgaactggataaaaccaagcgccgggaactcatgcttaatgaagtcatcaagcagacacaatccacattg |
| | | tccgcgctaattaatgatctaaattatatttctggaaagtttaacgacgccctggatgcgcataatcggcaaggagaaaaaattctaaaactccagaagatgatgaatgcattga |
| | | ccggtgcagcagatgaattgaataagaaatctaataaaatgcaaaacagtgcactcaaacttaagtgccctgtttatcttgatgaggccaaactcagttcgctgacccgagag |
| | | cccatcaaaaaacaattccatgatgttgttctttcattctacgaagaatatcttgttgaagagcaaaacgatgggaagagtgttgaagtgaaaaaacttaagatcaaagaaaacg |
| | | cttccactcagcaattagagaaacttgccgcgatctttgaaggcatcggctatttcagagcgggggatgttattaaaggcaaactaactgggctattctcataatgaaaaaacc |
| | | agatactcaggtatcggccttgctggtgcagaagcaccagcttgaacaaagcgagcatcaattgggtgaccttgatgctgctctagaagcgcttaacgctttgcaaactgata |
| | | ccgaagcttctttagatgaaatgattttggctatggatggtgttctggaacactcaggtatcacgtttgatgaggatatccacacaacggtttctagtgaattcagcgattaccttg |
| | | aatcctgtttgaccacgtcatcgtccagtatcagtaaactgtcgatgatagaaacaatagcgttcaccagcgatatggactgggaaacctattcccagtccatatcgcagtatgc |
| | | ccataaacacaatatcgatttaatagtcgatccgtttagcgccctgatgtctccaatccaaagaattgctctggaaaaacgtattcaggaagacttgaccttaaagactgcccgc |
| | | tgcgacaaatatgattacatgatcgctggcacctgtggcgttattggcggacttatcgatatttttctggtaggcgtacctggagcaggaaaactgacccagcttgcagataatg |
| | | cagtggacggtgccgttgagaaattcgcttcagcctttggatggaagggcagttcagaagcaagcgattcgacaaaaagcgctatcggttttctggagagaaaattcaaaat |
| | | caattatgaccatcggcatggcggagatgttgacggtttgttcaggatgaacacgaagaatcaccatattaaaagtctcgcccactccccggacttagtcggtttatttttctcga |
| | | tcctggatcaatttaccagtacggcacattttgtggcagacggaaaattggtttccgtagataccgagacttttgagcttaaagggaataacgttgtctctaaggtatttagtggttt |
| | | cgtaaactggctgggccaccttttctctgatatggcaggttcttccggtgcagcagggagaggctccggtatccccattcctttcttttcattacttcagtttattaatgtgggtgaa |
| | | tttggccagcatcgccagtctttcgcaaccgtcgccgtccaggtttttgagaaagggtatgacttacggcatggattagcgatggcgatccccgtcatgattactgagttgcttg |
| | | tgcgaatcacctggacggttaaacaacgttgctatcataagaaggactggggtgaatgtattccttcagcaaataaccctgaactcaggcgaatgttgcttgtggcgcatgga |
| | | accttgtgtctgatggatgtaggagatgcggcacttcgttcaggaggcgaaatgattcagttcctcctgagaacgaacctcatcggctggacgaggtttggaattctagcgatt |
| | | aaagaactccatgtctggtataaagcaggcggaattgatgccaatgctgtagatgaatatatggatcatgaacttcggcgaatgctaaaagcggggtagcgttacggctttgtt |
| | | gaataacattacgtttgggtgcttggctgtaaaaagctaggcaatggcgtatctgtcgacgcaatgcagaaaaggcaacttaattgcgaaacagaaatgttcggtgagttgctt |
| | | gaccgtcctatggcagctaagtgccagaagtcgacgttgctaacatcagtatgtactcatcggcacagtccatgtcagagctattaactatagataaaaattcaataattaataa |
| | | aataagaaccatctttctaggtggttcttattattaacaataaatattacgatttcaacgagggttagaatg (SEQ ID NO: 412) |
| 48 | TerB + | cctggtcctgccaattgctcccccagccatatgacataatccttttgaataatagggtttttatgcttgtactctagcccattcgcggtatcattttacgatctctcttccagttttatgc |
| | DUF279 + | ttaccgcctttgcctatcgtagaacaatgccgggaagcgttatcagcgattaagggcaaggaatgggcttctggatatttgttattatgctggcggttatctggcttctgttttcca |
| | Lhr | aaaagaaaaaatcgccgccccccagagtaaacaacaaaatcatcaccaaaataaatcattcatctcgacagaaatctctcaataagccagataacagcatgacaaatatgca |
| | helcase | ttctcaggcctccgatgatgacgaactggcaacctttacttttgtgaacgggcagacggttgaatacagcaccagccgccagccgtcacgagaaaacgccgcccgtagcaa |
| | | taccactccagcgcgatgggtcaaaccgggagaaagcatcaccattcaaaatgtcgtcattaatcacggttatttttatttcggcgggcggttaaaaacacattcatcaggaga |
| | | atatggatatctttataacgatgactccgacgcttcgctggttaatgacgcttttcccatcgagcctggttcacggcattattatgatgagtcactgggatactggcccagctttgc |
| | | cacactctcccctcgctgccgtggcgcctatcttgactggctggcaagcgatcgcagcgatgcgagctgccccgttggctatgtttttatctatttttacggtctagaacgccgc |
| | | gtactggccgatggcacacaagaagccatttctgacgatgaattcaaagcattattcgaagagatatcgcgcctgagaaccgtatttcaggcaagcggttccttccggcattat |
| | | gcaacgcagttgctggaaatgatgatcgttctccgaccgaagttgctttctatatataccgaaaacgaatatttctcatcgaggagttcattactgttcagattaaatctagcgact |
| | | gtggtcgataaaggacaacctatttgtgccgctctggcactggcatggatatactattttcctgattacaccctgcgcacgcctgcccgtcgatgtcatgctgaattttccgcatt |
| | | attcaaacagcgttatactcaaaaatacggtgacggtattgtcgtcaaacccaataaaacacggttgtatttaagctatacccccgccagtggtacgcttcgggaacttcaggta |
| | | aaaaaacagatggatcttcccgatcccagcgttttaaaagccccagttcagaaattaatttctgttgcagaatcctgtatcaacgcgctggatgcctacagtcgctatctcggta |
| | | aaaaagatgcctcaccaagtgatgtcgccgccatcatgctgcttcccgatgaaatactgaccgaagatgcagaacgtctatttgctgaatttaaacactgggcagatgagaaa |
| | | atccgtgaacattcaggactggcgacagtggctgatttctgggccagactgggtatgcctgtaccggataagattaataagaaagaagccgagctgatgcaaaatttcgccc |
| | | ggcgagcaggctacggcattgcgccggatatgcgctatcaccttgtcagaccggatccagaaggtcatcttgttttatttcctgaagggcatgcggaattctacgtaccgtcg |
| | | gcggaatttacgtcagtctctgtggcgcttcggttgggtgccatgattgcacaaatggacaagcgcgtggatgttgctgaacaggccgcgctggagaaaacgattaatcata |
| | | acgatgcgctgtcgccaacagaaaaacgttcgctgcacgcctacctcacctggcggctcaatacgcctgcaaatcaggctggtctgaaaggtaaaattgagcaactcagcg |
| | | ataaagataaatccactattggcaacgtgattatcagcgtcgcctgcgcagatggaaaaatcgatccggctgaaatcaaacaactggaaaaaatctacgccagcctcggtct |
| | | ggacagcagtgccgttaccagcgatatccaccgactgtcaaccgcagaaacaactccgacagctacgttacaaaccccatcagcgacgagcggcgcgttttctcttgatga |
| | | acggatccttgcccgtcatgaatccgacacaacggacgtacgccagttactgaacaccatcttcaccgaagatgaacccgcagacgaatccccagcggagatcccgccac |
| | | acgctggcgcaggtcttgatgaagcacatcatcaactttaccaacgtttgcaggaaaaagaacgctgggcgcgaaacgaagtcgctgagctatgccagcagtttaatttgat |
| | | gctaagcggcgcgattgaagcaattaatgactggtctttcgaacaggttgacgccccggtgcttgatgatgacgatgatatttacgttgacctggaaattgcacaagaactcaa |
| | | aggataatttatgtctggcattcgtattcgtctcaaagaaagagacgctattattcagtcactgaagtcaggtgttacgcctaaaattggtattcagcacattcaggttggccgggt |
| | | caacgaaataaaagcgctgtatcaggatattgagcgtatcgctgatggcggcgcaggattccggctgattattggggaatatggctcaggtaagacattctttttaagcgttgt |
| | | gcgctcaattgcgctagaaaaaaagctggtgacaatcagcgccgatttatccccggacaggcgcatccacgcgacgggtgggcaggcgcgtaacctctactccgagcta |
| | | atgaaaaatctatccacccgaaataagccggatggaaacgcattattaagcgtggttgagcgctttatcacggaagccagaaaagaagcagaaagtacaaatgtgtcagttc |
| | | cgacgattattcaccaaaagctcgccgccctgtctgatatggttggcggttacgatttcgccaaagtcattgaatgttactggcagggccacgagcaggataatgagacattg |
| | | aaatcaaatgccatccgctggctaagaggtgaatacaccacgaaaaccgacgcccgtaacgatctgggtgtgcgcaccattatttctgatgcctctttctacgattcgctaaag |
| | | ctgatgagcctgtttgtccgtcaggccggatacgcgggtctgctggtgaatctggatgagatggtcaatctgtataagctcagtaacactcaggcccgcgttgccaactatga |
| | | acagatactgcgtattctgaatgactgcctgcaagggacggctgaatatatcggttttttacttggcggtacgccagaattcctgttcgatccgcgcaaggggttgtacagctac |
| | | gaagcgctccagtcccgactggcggaaaatagcttcgctcagcgggctggtgtcattgattattcgtccccttccctgcacttagccagcctgacgccggaagaactctatatt |
| | | ctgttgaaaaaccttcgtcacgtttattccggcggcgatgcggataagtatctggttcctgatgatgctctgacggcatttttacgccactgtagcaacactattggcgatgcctat |
| | | ttccgtacgccacgaaacacgattaaagccttcctggatatgctggccgtgctggaacaaaacccatccattcagtggtcacagttaatcgccggtgtcgcgatcgcggaag |
| | | aaaaacccagtgatatggatgaaataacatcggcagaagatgccgatgaggacggtctggccgacttcagattatgatgaacgaataccagcggctggatccacggatac |
| | | agaagtggatataccggcagggatgggccgatctcagggaactgcaaaaaaaatccgtttcaccgatattagcgggcgatcgggatgttctgatcagcgccgcgactgcc |
| | | gcaggtaaaacagaagcgtttttcctgcccgcctgttctgccattgcggatattcagggcggctttggcattttatacatcagcccgcttaaggccctgattaacgatcagtatc |
| | | gaaggctggaaaacctcggtgatgcgttggagatgccggtcacgccctggcatggtgatgttgcgcagagcaaaaagctgaaagcaaagaagaatcctgccggtattttg |
| | | cttatcaccccggaatcgctggaagcgatgctgatccgcaatgcgggatggttaaagcaggctttcgcgccactggcatatatcgccattgatgaattccatgctttcatcggtt |
| | | ctgagcggggtatgcagcttctctctctgttaaatcgagtcgatcacctgctgggaagaatcaacaatccagtcccccgagtcgcactcagcgcaacgctgggggaactgg |
| | | aacaggtgccgttatctctgcggccaaatcaacgtctgccctgtgacattattaccgacagtcagactcacgccacgctaaaagtacaggtgaaaggttatctggaaccgctg |
| | | accacctcgggccagcaatctccaccgtcggcagagacgcaaatctgccatgatatctttcgcctctgtcgtggtgattcccatctggtgttcgctaatagtcgcaaacggac |
| | | cgaaagcattgccgccacgcttagcgatctcagtgaagcgagcatcgttcccaatgagttctttccccatcacggatctctgtccagagatctgcgtgaaacgctggaacaga |
| | | ggcttcaacaaggcaacttacccaccaccgccatctgtacgatgacgttagagcttggcatcgacatcggtaaagtcagctccgttgtgcaagttaccgccccccattccgta |
| | | gccagcctgcgtcagcgaatgggacgctccggtcggcgcgactcgcctgccgtattgagaatgctgattgccgaacatgaactgacgccaacatcaggcattgtcgacca |
| | | gctcaggcttcagcttgttcagtcgctggccatgatccgcttacttatcggcaacaaatggtttgagccagctgatacccggcagatgcactattccaccctgttccatcagatc |
| | | ctggcgatcgtggcgcagtggggaggcgtgcgtgcggatcagatctggtcacagctatgcctgcaagggccatttcagaaagtccggatctatgacttcaaaacgttattga |
| | | aacatatgggggagcaccagtttctgacccagctctcaagcggcgaactggttctgggcgtcgagggcgaacgtcaggtaaatcaatacaccttctacgccgtgttcagca |
| | | cgccggaagagtttcgcattgtggcggggagcaaaacactgggctccattcccgttgattccccactgatgcctgatcaacacattattttcggcggtcgacgctggaaggta |
| | | accgatatcgatagtgataaaaaagttatttatgtcgaggcgacaaagggtgggcagccgccgttatttggcggacaagggatgtccattcatgatgtcgtccgccaagaaat |
| | | gctcactatttatcgggaaggcgactaccgcatcaccgttggcaatcgcaaggccgattttgccgataccacggccaaaaacctgtttgatgaagggctgcactgttttcgca |
| | | acaataatctggcttcggaatgttttattcagcagagacagcatgtctacattcttccctggctaggcgatcaaaccgtaaacacgttgtcggcattacttatccaacgcggtttc |
| | | aaggcgggctcatttgctggtgtggttgaagtagaaaaaactacggtctcggaggttaaacaagcgttattcagcgcacttcaggaagggctaccttacgaatcccgtcttgc |
| | | cgaaagcatcgttgaaaagtgcctcgaaaaatatgatgagtatttacccgagacgttgctgacgcaggaatatggattacgtgcttttaatattgaacgcgtgacggagtggtt |
| | | gcaggggcatttatattaaggggaagaaga (SEQ ID NO: 413) |

TABLE 17
Genome coordinates of RADAR editing sites in Figure 27

| | | Position in genome | % A-to-I |
|---|---|---|---|
| | | (Genbank: | RNA |
| Site # | Gene | GCA_000005845.2) | editing |
| 1 | ffs | 476502 | 82 |
| 2 | dinQ | 3647752 | 88 |
| 2 | dinQ | 3647753 | 57 |
| 3 | ftsI | 92547 | 90 |
| 4 | lpp | 1757597 | 52 |
| 5 | rpsB | 190414 | 76 |
| 6 | ssrA | 2755713 | 61 |
| 6 | ssrA | 2755714 | 56 |
| 7 | (intergenic) | 3647944 | 69 |
| 7 | (intergenic) | 3647945 | 97 |
| 8 | hokB | 1492029 | 95 |
| 9 | mgrR | 1622894 | 87 |
| 9 | mgrR | 1622895 | 87 |
| 10 | ptsI (1) | 2534135 | 80 |
| 11 | secY | 3443842 | 78 |
| 12 | atpC | 3915927 | 69 |
| 12 | atpC | 3915928 | 76 |
| 13 | rbsB (1) | 3937080 | 76 |
| 14 | rpoA | 3440833 | 74 |
| 15 | rplI | 4426356 | 73 |
| 16 | (intergenic) | 2002020 | 70 |
| 17 | pflB | 951380 | 68 |
| 17 | pflB | 951381 | 58 |
| 18 | ptsI (2) | 2534211 | 68 |
| 19 | rplA (1) | 4179468 | 66 |
| 19 | rplA (1) | 4179469 | 68 |
| 20 | (intergenic) | 127818 | 68 |
| 21 | skp | 200777 | 67 |
| 22 | (intergenic) | 2518138 | 51 |
| 22 | (intergenic) | 2518139 | 66 |
| 23 | rbsB (2) | 3937116 | 65 |
| 24 | infC | 1800153 | 65 |
| 25 | rplT | 1799499 | 64 |
| 26 | gapA (1) | 1863658 | 64 |
| 27 | sodB | 1735694 | 62 |
| 28 | gapA (2) | 1862864 | 61 |
| 29 | rpsC | 3449386 | 61 |
| 30 | leuW | 697012 | 61 |
| 31 | rpsA | 962878 | 60 |
| 32 | ibsC | 3056901 | 60 |
| 33 | ahpC | 639397 | 59 |
| 33 | ahpC | 639398 | 56 |
| 34 | oxyS | 4158372 | 59 |
| 35 | rpmG | 3811305 | 58 |
| 36 | (intergenic) | 780980 | 57 |
| 37 | iscU | 2660065 | 57 |
| 38 | ryfD | 2734233 | 56 |
| 39 | deaD | 3306635 | 56 |
| 40 | hns | 1292675 | 56 |
| 41 | (intergenic) | 4392565 | 56 |
| 42 | tig | 456390 | 56 |
| 42 | tig | 456391 | 56 |
| 43 | rplA (2) | 4178970 | 56 |
| 44 | tsf | 191433 | 51 |
| 44 | tsf | 191434 | 55 |
| 45 | rnpB | 3270434 | 54 |
| 46 | (intergenic) | 781019 | 54 |
| 46 | (intergenic) | 781020 | 52 |
| 47 | eno | 2906708 | 52 |
| 48 | (intergenic) | 3071334 | 51 |

TABLE 18A
Description of phage T2 fragments in FIGS. 28C-28E

| Fragment | Length | A93% | | A121% | | Gene | | | |
|---|---|---|---|---|---|---|---|---|---|
| # | (bp) | editing | | editing | | # | Accession | Gene | Description |
| 1 | 2392 | 28 | 23 | 37 | 32 | 1 | AYD82599.1 | rIIA.1 | hypothetical protein |
| | | | | | | 2 | AYD82598.1 | rIIA | protector from prophage-induced early lysis |
| 2 | 1818 | 5 | 5 | 6 | 6 | 1 | AYD82600.1 | gp39 | DNA topoisomerase II large subunit |
| 3 | 261 | 6 | 6 | 8 | 9 | 1 | AYD82601.1 | gp39.1 | hypothetical protein |
| 4 | 1423 | 8 | 5 | 10 | 8 | 1 | AYD82606.1 | — | hypothetical protein |
| | | | | | | 2 | AYD82605.1 | cef | modifier of suppressor tRNAs |
| | | | | | | 3 | AYD82604.1 | goF | mRNA metabolism modulator |
| | | | | | | 4 | AYD82603.1 | gp39.2 | hypothetical protein |
| | | | | | | 5 | AYD82602.1 | — | hypothetical protein |
| 5 | 3570 | 6 | 9 | 7 | 11 | 1 | AYD82613.1 | srd | anti-sigma factor |
| | | | | | | 2 | AYD82612.1 | dda.1 | hypothetical protein |
| | | | | | | 3 | AYD82611.1 | dda | DNA helicase |
| | | | | | | 4 | AYD82610.1 | dexA.2 | hypothetical protein |
| | | | | | | 5 | AYD82609.1 | dexA.1 | hypothetical protein |
| | | | | | | 6 | AYD82608.1 | dexA | exonuclease |
| 7 | 1339 | 38 | 44 | 49 | 56 | 1 | AYD82628.1 | — | hypothetical protein |
| | | | | | | 2 | AYD82627.1 | dam | DNA adenine methyltransferase |
| 8 | 201 | 4 | 2 | 5 | 3 | 1 | AYD82629.1 | — | hypothetical protein |
| 9 | 442 | 1 | 1 | 2 | 2 | 1 | AYD82635.1 | dmd | discriminator of mRNA degradation |
| | | | | | | 2 | AYD82634.1 | gp61.4 | hypothetical protein |
| 10 | 2956 | 22 | 20 | 29 | 27 | 1 | AYD82638.1 | uvsX | RecA-like recombination protein |
| | | | | | | 2 | AYD82637.1 | gp40 | head vertex assembly chaperone |
| | | | | | | 3 | AYD82636.1 | gp41 | helicase |
| 11 | 2697 | 2 | 2 | 3 | 3 | 1 | AYD82644.1 | gp43 | DNA polymerase |
| 12 | 687 | 3 | 3 | 5 | 4 | 1 | AYD82648.1 | gp45 | sliding clamp |
| 13 | 588 | 85 | 85 | 93 | 92 | 1 | AYD82650.1 | gp45.2 | hypothetical protein |
| | | | | | | 2 | AYD82649.1 | rpbA | RNA polymerase binding protein |
| 14 | 1203 | 52 | 46 | 59 | 53 | 1 | AYD82657.1 | a-gt | DNA alpha glucosyl transferase |
| 15 | 545 | 27 | 22 | 48 | 40 | 1 | AYD82664.1 | gp55.2 | hypothetical protein |
| | | | | | | 2 | AYD82663.1 | gp55.1 | hypothetical protein |
| 16 | 3394 | 60 | 57 | 69 | 67 | 1 | AYD82674.1 | gp49 | recombination endonuclease VII |
| | | | | | | 2 | AYD82673.1 | nrdD | anaerobic ribonucleotide reductase subunit |
| | | | | | | 3 | AYD82672.1 | nrdG | anaerobic NTP reductase small subunit |
| | | | | | | 4 | AYD82671.1 | — | hypothetical protein |
| | | | | | | 5 | AYD82670.1 | gp55.8 | hypothetical protein |
| | | | | | | 6 | AYD82669.1 | nrdH | glutaredoxin |
| 18 | 2329 | 3 | 2 | 5 | 3 | 1 | AYD82686.1 | nrdC.5 | hypothetical protein |
| 19 | 528 | 5 | 5 | 8 | 8 | 1 | AYD82689.1 | nrdC.8 | hypothetical protein |
| 20 | 303 | 2 | 1 | 3 | 2 | 1 | AYD82690.1 | nrdC.9 | hypothetical protein |
| 21 | 2659 | 30 | 31 | 33 | 36 | 1 | AYD82699.1 | mobD.2 | hypothetical protein |
| | | | | | | 3 | AYD82693.1 | nrdC.11 | hypothetical protein |
| 22 | 902 | 6 | 6 | 7 | 7 | 1 | AYD82706.1 | rI.1 | hypothetical protein |
| | | | | | | 2 | AYD82705.1 | rI | lysis inhibition regulator |
| | | | | | | 3 | AYD82704.1 | rI.-1 | hypothetical protein |
| 23 | 2602 | 4 | 4 | 6 | 7 | 1 | AYD82724.1 | ip4 | hypothetical protein |
| | | | | | | 2 | AYD82721.1 | vs.7 | hypothetical protein |
| | | | | | | 3 | AYD82720.1 | vs.6 | hypothetical protein |
| | | | | | | 4 | AYD82719.1 | vs.5 | hypothetical protein |
| | | | | | | 5 | AYD82718.1 | vs.4 | hypothetical protein |
| | | | | | | 6 | AYD82717.1 | vs.3 | hypothetical protein |
| 24 | 495 | 6 | 5 | 10 | 8 | 1 | AYD82725.1 | e | lysozyme murein hydrolase |
| 25 | 594 | 7 | 5 | 9 | 8 | 1 | AYD82730.1 | e.6 | hypothetical protein |
| 26 | 177 | 3 | 3 | 4 | 4 | 1 | AYD82731.1 | — | hypothetical protein |
| 27 | 264 | 3 | 2 | 4 | 3 | 1 | AYD82732.1 | e.8 | hypothetical protein |
| 28 | 351 | 7 | 6 | 10 | 10 | 1 | AYD82733.1 | — | hypothetical protein |
| 29 | 402 | 5 | 4 | 8 | 5 | 1 | AYD82734.1 | trna.1 | hypothetical protein |
| 30 | 991 | 2 | 2 | 6 | 4 | 1 | AYD82737.1 | trna.4 | putative membrane protein |
| | | | | | | 2 | AYD82736.1 | trna.2 | hypothetical protein |
| | | | | | | 3 | AYD82735.1 | — | hypothetical protein |
| 31 | 309 | 6 | 5 | 8 | 9 | 1 | AYD82738.1 | ip7 | hypothetical protein |
| 32 | 255 | 20 | 19 | 26 | 25 | 1 | AYD82739.1 | ip5 | hypothetical protein |
| 33 | 1423 | 28 | 27 | 36 | 36 | 1 | AYD82742.1 | gp1 | deoxynucleoside monophosphate kinase |
| | | | | | | 2 | AYD82741.1 | gp57A | chaperone for tail fiber formation |
| | | | | | | 3 | AYD82740.1 | gp57B | hypothetical protein |
| 34 | 1277 | 54 | 54 | 69 | 72 | 1 | AYD82745.1 | gp50 | head completion protein |
| | | | | | | 2 | AYD82744.1 | gp2 | DNA end protector protein |
| 35 | 8107 | 2 | 2 | 3 | 3 | 1 | AYD82755.1 | gp9 | baseplate wedge tail fiber connector |
| | | | | | | 2 | AYD82756.1 | gp10 | baseplate wedge subunit and tail pin |
| | | | | | | 3 | AYD82757.1 | gp11 | baseplate wedge subunit and tail pin |
| | | | | | | 4 | AYD82758.1 | gp12 | short tail fibers protein |
| | | | | | | 5 | AYD82759.1 | wac | fibritin |
| | | | | | | 6 | AYD82760.1 | gp13 | neck protein |
| | | | | | | 7 | AYD82761.1 | gp14 | neck protein |
| 36 | 5149 | 33 | 37 | 46 | 50 | 1 | AYD82762.1 | gp15 | tail sheath stabilizer and completion protein |
| | | | | | | 2 | AYD82763.1 | gp16 | small terminase protein |
| | | | | | | 3 | AYD82764.1 | gp17 | large terminase protein |
| | | | | | | 4 | AYD82765.1 | gp18 | tail sheath protein |
| 37 | 492 | 4 | 4 | 6 | 6 | 1 | AYD82766.1 | gp19 | tail tube protein |
| 38 | 1284 | 2 | 3 | 3 | 4 | 1 | AYD82773.1 | gp24 | capsid vertex protein |
| 39 | 1476 | 35 | 33 | 45 | 40 | 1 | AYD82863.1 | gp24.3 | hypothetical protein |
| | | | | | | 2 | AYD82775.1 | gp24.2 | hypothetical protein |
| 40 | 1807 | 17 | 23 | 23 | 30 | 1 | AYD82776.1 | inh | inhibitor of prohead protease |
| 41 | 832 | 1 | 3 | 2 | 3 | 1 | AYD82781.1 | uvsY | recombination, repair and ssDNA binding |
| | | | | | | | | | protein |
| | | | | | | 2 | AYD82780.1 | uvsY.-1 | hypothetical protein |
| | | | | | | 3 | AYD82779.1 | uvsY.-2 | hypothetical protein |
| 42 | 1025 | 1 | 1 | 2 | 2 | 1 | AYD82783.1 | gp26 | baseplate hub subunit |
| | | | | | | 2 | AYD82782.1 | gp25 | tail lysozyme |
| 43 | 6240 | 1 | 1 | 1 | 1 | 1 | AYD82784.1 | gp51 | baseplate hub assembly protein |
| | | | | | | 2 | AYD82785.1 | gp27 | baseplate hub subunit |
| | | | | | | 3 | AYD82786.1 | gp28 | baseplate hub distal subunit |
| | | | | | | 4 | AYD82787.1 | gp29 | baseplate hub subunit tail length |
| | | | | | | | | | determinator |
| | | | | | | 5 | AYD82788.1 | gp48 | baseplate subunit |
| | | | | | | 6 | AYD82789.1 | gp54 | baseplate subunit |
| 44 | 291 | 1 | 1 | 2 | 2 | 1 | AYD82790.1 | alt.-3 | hypothetical protein |
| 45 | 4155 | 2 | 2 | 3 | 3 | 1 | AYD82792.1 | alt | ADP-ribosyltransferase |
| | | | | | | 2 | AYD82791.1 | alt.-1 | hypothetical protein |
| 46 | 366 | 6 | 7 | 8 | 9 | 1 | AYD82801.1 | gp30.7 | hypothetical protein |
| 47 | 177 | 6 | 6 | 9 | 9 | 1 | AYD82802.1 | gp30.9 | hypothetical protein |
| 48 | 249 | 2 | 3 | 3 | 4 | 1 | AYD82803.1 | rIII | lysis inhibition accessory protein |
| 49 | 336 | 1 | 2 | 2 | 2 | 1 | AYD82804.1 | gp31 | head assembly cochaperone with GroEL |
| 50 | 1698 | 4 | 3 | 6 | 4 | 1 | AYD82809.1 | cd.2 | hypothetical protein |
| | | | | | | 2 | AYD82808.1 | cd.1 | hypothetical protein |
| | | | | | | 3 | AYD82807.1 | cd | deoxycytidylate deaminase |
| | | | | | | 4 | AYD82806.1 | gp31.2 | hypothetical protein |
| | | | | | | 5 | AYD82805.1 | gp31.1 | hypothetical protein |
| 51 | 276 | 3 | 3 | 5 | 5 | 1 | AYD82810.1 | cd.3 | hypothetical protein |
| 52 | 3683 | 5 | 6 | 7 | 8 | 1 | AYD82823.1 | td | thymidylate synthetase |
| | | | | | | 2 | AYD82822.1 | nrdA.2 | hypothetical protein |
| | | | | | | 3 | AYD82821.1 | nrdA.1 | hypothetical protein |
| | | | | | | 4 | AYD82820.1 | nrdA | ribonucleoside-diphosphate reductase |
| | | | | | | | | | subunit alpha |
| 53 | 1448 | 45 | 62 | 58 | 69 | 1 | AYD82827.1 | frd.1 | hypothetical protein |
| | | | | | | 2 | AYD82826.1 | — | hypothetical protein |
| | | | | | | 3 | AYD82825.1 | frd | dihydrofolate reductase |
| | | | | | | 4 | AYD82824.1 | — | hypothetical protein |
| 54 | 366 | 1 | 2 | 2 | 3 | 1 | AYD82828.1 | frd.2 | hypothetical protein |
| 55 | 228 | 11 | 11 | 16 | 16 | 1 | AYD82829.1 | frd.3 | hypothetical protein |
| 56 | 909 | 2 | 3 | 3 | 4 | 1 | AYD82830.1 | gp32 | single-stranded DNA binding protein |
| 57 | 2162 | 40 | 48 | 51 | 67 | 1 | AYD82834.1 | rnh | RnaseH |
| | | | | | | 2 | AYD82833.1 | dsbA | double-stranded DNA binding protein |
| | | | | | | 3 | AYD82832.1 | gp33 | late promoter transcription accessory protein |
| | | | | | | 4 | AYD82831.1 | — | hypothetical protein |
| 58 | 4997 | 3 | 2 | 5 | 3 | 1 | AYD82835.1 | gp34 | long tail fiber proximal subunit |
| | | | | | | 2 | AYD82836.1 | gp35 | hinge connector of long tail fiber proximal |
| | | | | | | | | | connector |
| 59 | 417 | 42 | 48 | 46 | 54 | 1 | AYD82859.1 | — | hypothetical protein |
| | | | | | | 2 | BBC14887.1 | ndd.6 | putative outer membrane protein |
| | | | | | | 3 | AYD82858.1 | ndd.5 | putative outer membrane protein |
| 60 | 1166 | 26 | 27 | 29 | 31 | 1 | AYD82862.1 | rIIB | protector from prophage-induced early lysis |
| | | | | | | 2 | AYD82861.1 | denB.1 | hypothetical protein |

TABLE 18B
DNA sequences of fragments #1-60 in Table 18A

| Frag- | |
|---|---|
| ment | |
| # | DNA sequence |
| 1 | atgaaatcatatagagtaaatttagaactttttgataaagcagttcatcgagaatatagaatcattcaacgctttttcgatatgggagaagccgaagaatttaaaaaccgctttaaggatattagag |
| | ataaaattcaatccgacaccgcaactaaagatgaattactagaagttgctgaagttattaagcgtaatatgaattaatgaggaaattatgattatcaccactgaaaaagaaacaattcttggtaat |
| | ggttctaaatcaaaagcatttagcatcacagcatctcctaaagtatttaaaattctgtcatctgatttgtatacaaacaaaattcgcgcagtagtccgtgaattgattactaacatgattgatgccca |
| | tgctctcaatggaaatcctgaaaaatttatcattcaagttccaggacgattagatccgcgatttgtttgtcgagattttggtccgggtatgagtgattttgatattcagggtgatgataattctcctgg |
| | gctgtataattcatacttcagttcatctaaagctgaatctaatgatttcattggtggatttggtttaggttctaaatctccgtttagttatactgatacgtttagtattacttcataccataaaggtgaaatt |
| | cgtggttatgtagcttacatggatggtgatggcccacagattaaacctacattcgtaaaagaaatgggtccagatgataaaactggcattgaaatcgtagttccagttgaagaaaaagacttta |
| | gaaactttgcttatgaagtttcttatatcatgcggccgttcaaagatttggctatcattaatagtcttgaccgtgaaattgactattttccggattttgatgattattacggcgtaaatccagaaagata |
| | ctggcctgatcgtggtggattatatgctatctatggcggtattgtttatcctattgatggtgttattagagaccgcaactggttaagcattcgcaatgaagtgaattacattaagtttccaatgggttc |
| | acttgatattgctccatctcgcgaggctctttcacttgatgatcgtactcgtaaaaatattattgagcgagttaaagaactcagtgagcaagcatttaatgaagatgtaaaacgatttaaagaatct |
| | acatctcctcgtcacacatatcgtgaattgatgaagatggggtattctgctcgagattatatgattagtaattcagtcaaattcacgactaaaaatctgtcatataagaagatgcagagtatgtttg |
| | aacctgatagtaagttatgcaatgcaggagttgtgtatgaagtaaatcttgaccctcgactgaagcgcattaagcaaagtcatgaaacttcagccgttgcatcaagttatcgtctgtttggtatta |
| | atacaacaaaaattaatattgttattgataatattaaaaatcgtgttaatattgtccgtggattagcacgtgcgttagatgatagtgaatttaataacactttgaatattcatcacaatgagcgtcttct |
| | gtttattaacccagaagtagaatcgcagattgatttgcttcctgatattatggcaatgtttgaaagtgatgaagttaacattcattatttgtcagaaatcgaagctttagttaaaagctatattccaaa |
| | ggtagttaaaagtaaagctcctcgtcctaaagctgctacagcatttaagtttgaaattaaagacgggcgctgggaaaaagaggaactatttacacttacgtcagaagcagatgaaattactgg |
| | ttatgtagcgtatatgcatcgttctgatattttctctatggatggtactacatctctttgtaatccatctatgaatattttgattcgtatggctaatcttattggcattaatgaattttatgttattcgtccgctt |
| | ttacagaaaaaggtaaaagaactcggtcagtgccaatgtatttttgaaactctacgcgatttatatgtagatgcttttgatgatgtagattatgataagtatgtaggttattcaagttcagctaaacg |
| | atatattgataaaattatcaagtatcctgagctagattttatgatgaagtacttcagtgtagatgaagtttctgaagaatatacacgactcgctaatatggttagttcattacagggtgtatattttaat |
| | ggtggaaaagataccattggtcatgacatctggacagtaactaatctttttgatgtattatcaaataatgcttcaaaaaacagtgataaaatggttgctgagtttaccaagaaattccgtattgtttc |
| | cgacttcatcggatatcgcaactctttaagtgatgatgaagtttctcaaatcgctaaaactatgaaggcccttgcggcctaa (SEQ ID NO: 414) |
| 2 | atgattaagaatgaaattaaaattctgagcgatattgaacacatcaaaaagcgtagtggcatgtatattggctcttctgctaatgaaatgcatgagcgctttctgtttggtaaatgggaaagtgttc |
| | agtatgtacctggtcttgttaagcttattgatgaaattatcgataactcagtagatgaaggtattcgtactaagtttaaattagcaaataaaattaatgttactattaaaaacaatcaagtaacagttg |
| | aagataacggtcgtggtattccacaagcgatggttaaaacacctactggtgaagaaattcctggtccagttgctgcatggactattccaaaagcaggtggtaactttggtgatgataaagaac |
| | gcgtcaccggtggtatgaatggtgttggttctagtttgacaaacattttttctgtgatgtttgtcggtgaaactggcgatggtcaaaataatattgtagttcgttgttcaaatggcatggaaaataaa |
| | tcatgggaagatattcctggaaaatggaaaggaactcgtgttactttcattcctgattttatgtcatttgaaactaatgagctgtcccaagtttatcttgacattacacttgatcgtctccagacgctt |
| | gctgtagtttatcctgatattcaatttacctttaatggtaaaaaggttcagggcaattttaagaaatatgcacgacagtatgatgaacatgctattgttcaagaacaagaaaattgttctattgcggtt |
| | ggtcgttcaccggatggttttcgtcagttgacgtacgtcaataacattcatactaagaatggtggccatcatattgactgtgttatggatgatatttgtgaagaccttattccacaaatcaaacgta |
| | aattcaaaattgatgtaactaaagcacgtgttaaagaatgtttgactatcgttatgtttgttcgcgatatgaaaaacatgcgatttgactctcaaactaaagaacgacttacttctccttttggtgaaa |
| | ttcgtagtcatattcaacttgatgctaaaaagatttcacgcgctattctaaataatgaagcaattttaatgccaattattgaagcagcattagctcgtaaattggcggcggaaaaagcagcagag |
| | acaaaggcagctaaaaaagcttctaaagctaaggttcataaacatatcaaagcgaatctttgtggtaaagatgctgatactactcttttcttgactgagggtgattctgctatcggatatcttattg |
| | atgttcgtgataaagaacttcatggtggttatccattgcgtggtaaagttcttaatagctggggtatgtcatatgccgatatgcttaaaaacaaagaactatttgatatttgcgcaatcactggtcta |
| | gttcttggtgaaaaagctgaaaacttgaattatcataatattgctattatgactgatgctgaccatgatggtctaggaagcatttatccttctctgctcggattttttagtaattggccagaattgtttg |
| | agcaaggacgaattcgctttgtcaaaactcctgtaatcatcgctcaggtcggtaaaaaacaagaatggttttatacagtcgctgaatatgagagtgccaaagatgctctacctaaacatagcat |
| | ccgttatattaaaggacttggctctttggaaaaatctgaatatcgtgaaatgattcaaaatccagtatatgatgttgttaaacttcctgagaactggaaagagctttttgaaatgctcatgggagat |
| | aatgctgaccttcgtaaagaatggatgagccagtag (SEQ ID NO: 415) |
| 3 | atgaaatatattaatcgttctatcgcagcattagtattagcagtgtctttagtaggatgtactgatgctgataatgcaacaaaagttttgtcttcaagtggttttactaatattgaaatcactggatata |
| | attggtttggttgctctgaaaatgatttccagcatactggatttcgtgctattggacctaccgggcagaaagtagaaggaacagtatgttctggtttattcttcaaagattcgactatccgttttaaat |
| | aa (SEQ ID NO: 416) |
| 4 | atggaaaacttaattatcatcgagcaatctttcaacgattatggtatggcttatggttatcgtgcgataatggaagattctcgtggatgtgttatcgatattgctgaatgtaaagatttactgcagctt |
| | ttgaagattgttcgcaaaaattgggattgtgaaaatattaaagttcgaattgttacagaagaagaaactgtttttcatgatgtaaaattcgctaaaggtgctgctactcttctgaaacgtatcgctcc |
| | actgttcaattaatgaggaaattataatgaaacgtaaaattgttcagaactgcactaatgatgaatttgaagatgtattattcgatccagatttggtagtagttcaaaaggaacacaccatcaagttt |
| | actcacttgacttcggtttatgtgtatgagaaagtcggtgataaacaaccaatttacggtgtatttcgtgaaattactgaagatggcacaacttactggaaggaaatttattaatggctattaaattt |
| | gaagttaataaatggtatcaatttaaaaataaacaagctcaagaaaattttattaaagaccatactgataacggaatctatgcacgccgtttaggtatgcatccttttaaaattttagatgttgattat |
| | ctttggcgtcctactaaaattgtgacatctactggcacagttggatatgcaacacacggtgatatccttgacgaaaactttatctggctttctactaacgaagctgggttctttgatgaagtggaaa |
| | atccatatcaggcagttgaagagcaagagcaggaagagaaagagcaagaacaaatagaagatttcacagaattcccagtaatgaaagttactattgaaaataatgaacaggcatggtcctt |
| | gtatcaaatgctgaaagcacactttaaggaataattatgccaatgtatgattataaatgccaatccgaagattgcgggcatgaatatgaaaaaattaaaaagatttctgaacgagaaaatgatgt |
| | ttgccctaaatgtcatcgtttgtctactcgtcggccttctgctcctaagcatgtgaatggtggtttttacgacttacttaaagggtaattatgtttaaaatcggtaagaaatattgcattcgtgaaggt |
| | gaagaacagaaatatctactttctgctagtaataggaatagttctattaatgctgtaatattgactagtgaatttatcgttgaagatatgaaaggtcataatgttacaatgattagtacagcatctgg |
| | aaatgatggaaaaattcttcatagttgtcagagtaatgttctaatttatgatgaagaatttgacttcttcaaagaagtttccgaagattttgattttgaatgtactattactatgaaatctggtgaccctc |
| | tttcttttacagttagatga (SEQ ID NO: 417) |
| 5 | atgaagctgcataatatgtctaataatcaaattcgtaaaattaaacgtcgtttagagcatactcaggcatctgctaaaagacgttctaaagattttaacttagacttcaattacattaagaacatttta |
| | gaccaaaaagtttgcgcttactcgggagaaccttttgataatcgtattgaaggagagaaattatcattagaacgttttgataataacgttggatacattaaagggaatgttattgcagtaaagaaa |
| | aagtataatacatttcgttctgattatactttagaggagttaattgaaaaacgtgatttgtttgctttgcgaattggtcgttcatctgcgaaaaaagttcataaactaaatttagatgaaaagaaatgg |
| | gctaaaatcaaaaagacttataatcaaattaaagctatacagaaaaaacgtgaaaaccgaattgaacacatttctcagattctaaatcaaaacagacctctgacattaagctaagaattatagc |
| | acttaaagctcgtattgatggttctcgtatagcagaaggcgctgaagttgttaaattgaacgttcttcttaaaggctcggattggaaaactgtgaaaaagttgtcagaagcagaaatgcaatatg |
| | atatgtgtgataaaattattcaaggtgtagagcggtatcaaaacttgtcttttattgataaacttaaactgaaaagaggatatccgctaaattgttcaatttttaaacttatccgaggataatatggttt |
| | atgtatatgcgatagtttaccgagacaaagacggatttacggcgccagttccgcttgatgaacatcgtcctgctgtattttttgaatggaagattgctgataaagtatttaccactcttaaagagca |
| | gtatcaactagctttaggtaagggaattccaagattagttgagactccacgcaagttttggtttaataaaatagaagttaaacatgttaagcctgatgtagacacacaaagattatatcggcgaat |
| | tttagatactgggcgtattgttagtataccaattgcagggaatttacgatgacatttgatgatttgaccgaaggtcaaaaaaatgcctttaacattgttatgaaggctattaaagaaaagaaacatc |
| | atgtaactattaatggacctgctggtaccggtaagactactcttactaagttcatcattgaagctttaatatctacgggtgaaactggtattattttagcagctcctactcatgcagctaaaaagatt |
| | ctttcaaaactatcatggaaagaagcgagtactattcatagtattcttaaaattaacccagtaacatacgaagaaaacgttctttttgaacaaaaagaagtaccagatttagctaaatgcagggta |
| | ttaatctgcgatgaagtgtcaatgtatgatagaaagctatttaaaattctgctttcaactatcccgccgtggtgtactataattggaataggcgataataagcaaattagacctgttgacccagga |
| | gaaaatactgcttatatcagtccattctttacacacaaagatttttatcagtgtgaactcactgaagttaaacgcagtaatgctcctattattgatgtagctactgacgttcgtaacggtaagtggatt |
| | tatgataaagttgttgacgggcatggagtacgtggatttactggtgataccgctttacgcgattttatggtaaattatttttcaatcgtcaaatctttagatgatttgtttgaaaatcgcgtaatggcat |
| | ttacgaataaatctgttgataagttaaatagcattattcgtaaaaagatttttgaaactgataaagattttattgttggtgaaattattgtaatgcaggaaccattaattaaaacatataaaattgatgg |
| | aaagcctgtgtcagaaattatttttaataacggacaattagttcgtattatagaagcagagtatacatcaacgtttgttaaagctcgtggtgttcctggagaatacttaattcgtcattgggatttaac |
| | agtagaaacttacggcgatgatgaatattatcgtgaaaagattaaaataatttcatctgatgaagaactatataagtttaacctatttttaggtaaaacagcagaaacttataaaaattggaacaaa |
| | ggtggaaaagctccatggagtgatttttgggatgctaaatcacagttcagtaaagtgaaagcacttcctgcatcaacattccataaagcgcaaggtatgtctgtagaccgtgctttcatttatac |
| | accttgtattcattatgcagatgctgaattggctcaacaacttctttatgttggtgttacccgtggtcgttatgatgtattttatgtatgattaaatttgaggaagctattcgtggaaataactaaagatc |
| | agttttatcttcttcaagataaagtaagcgaaatttatgaaattgctcatggtaaaaatcgtgaaactgtaaaaattgaatctagtaagttgatgcttcaattagaagaaattgaacgagatttaattg |
| | cgttagaattcttttgtggcgaagtgaaaactgttacaattaatgattatgttttaggcgaaattagctatctttatgaggcgattattaatgattgaattaagttggtgccagtttaaatctcttatgac |
| | aaatgttaaagctgtcattgaagaaaatcagggtcctgaaaatattactattcgcgaaaaagctttaaagatagtatacagtcttgaagaaatacaaaaagatattgaatctatggcaaaatttatt |
| | gatgagcctattaataaagtttatattcaagactatactgtaggtcaaattcgcgatttagcgaggaaagtttaatgtttgattttattatagattttgaaacaatgggaagtggtgaaaaagcagct |
| | gttattgatttggctgtaattgcttttgaccctaatccagaagtcgttgaaacattcgatgaattagtttcacgtggcattaaaatcaaatttgatttaaaaagccaaaaaggacatcgtctttttacta |
| | aaagcactatcgaatggtggaaaaatcaatctcctgaagctcgaaaaaatattgcaccatcagatgaagatgtaagcactatcgatggtattgcaaaatttaatgattacatcaatgcacataat |
| | atcgatccttggaaatctcaaggctggtgtcgtggaatgtcatttgattttccaattttagtcgatctcattcgtgatattcaacgccttaatggtgtatctgagaatgagcttgacacatttaagttag |
| | aaccatgtaaattctggaatcagcgtgatattcgtaccagaattgaagcacttctgcttgttcgtgatatgaccacgtgtcctcttccaaaaggaactttagatggattcgttgcgcatgattctatt |
| | catgactgtgcgaaagacatcctgatgatgaagtatgctttgcgatatgctatgggtcttgaagatgctccatcagaggaagaatgcgatcctctatctcttccaacaaaacgataa (SEQ |
| | ID NO: 418) |
| 7 | atgattaataaaattgtgcatgaaatggctttaaacggagattcatataaaatatctgccgtagttgaaaatttcatacttaataaagtaaaagaatatttcactgattgttcagttagttatcaagaa |
| | aaaatggttttaattgatgatactgaaaaatcaaataatttgttttgctctaattttataactaaaaagcgtactagaagatttgatattgttatttctcgcaacggtaaaaagcatataattgaaattaa |
| | acaccaagttggtggaggtacagctattgattcggttggaatatatttagaagataaagagaaattaaaagaatacacaaaaactgaaaaccctgtgtcattgatgatattagattttttgccatg |
| | cggatattatccacgtaataaatggacaaaaagagaatcatttactgataatccaaccatccaagcaaggtttaatgaatatgctaaatcacaaaacgtgttagtattattatcaaatacatatgat |
| | gaagaattgtataattcatttttgctgcaataaatgagagaatataatgctaggagctatcgcgtatacgggtaataaacaatcattattacctgaacttaagcctcactttccgaaatatgacaga |
| | ttcgtggatttattttgtggaggtttatcagtgtctttgaacgtcaatggtcctgtattggccaatgatattcaagaaccaattattgaaatgtataagcgtcttattaatgtatcatgggatgacgtttt |
| | aaaagtaataaagcaatacaaactatcaaaaacatcaaaagaagagtttttgaaattacgtgaagattataataaaactagagatcctcttttactttatgttcttcattttcacgggtttagtaatat |
| | gattcgtataaacgataaaggaaattttactactccgtttggaaaaagaactataaacaaaaatagtgaaaaacgctttaatcactttaaacaaaattgtgataaaataatctttagttcattgcattt |
| | taaagatgtcaaaattctagacggcgattttgtatatgtggaccctccgtatcttataacagttgctgattataataaattttggtcagaagaagaagaaaaagaccttttaaatcttttagattcttta |
| | aatgacagaggaataaaatttggactgtcgaatgttttagagcatcacggaaaggaaaacactcttcttaaagaatggtctaaaaaatataatgttaagcatcttaataaaaaatacgtctttaac |
| | atatatcattccaaagaaaagaatggaactgatgaagtatatatttttaattaa (SEQ ID NO: 419) |
| 8 | atggtacaaaaattaatggcacttgttaatgccatcaaaggtaataaaaagcgtatagcttttactatttctgctatggtaggaattttactctggaactttattttatcacctgttgcaattgcacatg |
| | gtattaatattccaatagttactcttgatacattcgtagatttagcatttgctttagttgggttaatttaa (SEQ ID NO: 420) |
| 9 | atggaattggtaaaggtagtttttatggggtggtttaagaatgaaagcatgtttactaaagaaaccacaatgatgaaagatgacgttcaatgggctactactcaatatgctgaagttaataaagc |
| | attagttaaagctttcattgatgataagaaagtgtgtgaagtggattgccgaggataatatgcatattgttttatttaaacctactccgtataacgtcaggaaaaatacgcaattcaaagcacttatt |
| | gcagatacgtgggaattggtgttagatattccagcagaagaaagtcctccatttggtcgagtggaatttattaagtttgctgttcgccctacgaagcggcagattcgccaatgcaaaagatactt |
| | tcgtaagatcgttaagctagagaaacagtttgtaacatgtgattacgcaaaagttttaaaataa (SEQ ID NO: 421) |
| 10 | atgtctattgcagatttaaaatcccgtttgattaaagcttccacttctaaaatgactgctgagctgactacatctaaattctttaatgaaaaggatgtaatccgtacaaaaatcccaatgcttaatatt |
| | gctatttctggtgcgattgatggtggtatgcagtctggtttaactattttcgcagggccttctaaacactttaaatcaaatatgtctttgactatggttgcggcatatttgaacaaatatcctgacgcg |
| | gtttgtctattctatgatagcgaatttggtattactccagcttatttgcgatccatgggagttgacccggaacgagtaattcatacgccaattcagtcagttgaacagctgaaaattgatatggtga |
| | accagcttgaagctattgagcgtggtgaaaaggttattgtattcatcgactcaatcggtaatatggcttctaagaaagaaacggaagatgccttgaatgaaaaatctgtggcagatatgactcg |
| | tgctaaatcactgaagtcattattccgtattgttactccttattttagcattaaaaatattccgtgtgttgcggttaaccatacaattgaaacaattgaaatgtttagtaaaaccgtgatgacaggtggt |
| | acaggcgtaatgtattcggctgatactgtattcattatcggtaaacgtcagattaaagatggttctgatcttcaggggtatcaatttgttctaaatgtagaaaaatctcgtaccgttaaagaaaaaa |
| | gtaaattttttattgatgttaaatttgacggtggtatcgatccttattctggattgttagatatggctctagaattaggattcgtggtaaaacctaagaatggttggtatgctcgtgaatttcttgacgaa |
| | gaaaccggcgagatgattcgcgaagaaaaatcttggcgtgcaaaagatactaactgcactacattctggggtcctttatttaagcatcaaccattccgagatgctattaaacgtgcttatcagtt |
| | aggtgctattgatagtaatgaaattgttgaagctgaagttgatgaattgattaactcaaaggttgaaaaatttaaatctccagaaagtaaaagtaaatcagcagctgatttagaaactgacctcga |
| | acagttaagtgatatggaagaatttaatgaataaagatgatttagatttagatctagaaattatcgatgaatccccctcttcggagggggaagaagaaagaaaagaacgtctttttaatgagtct |
| | cttaagataattaaatccgctatggaaaatgttatccaggagattgtcattaaactagaagatggttctacacatatagtgtatgtaacaaaactggattgggttgatggaaaggttgtaatggac |
| | tttgctgttcttgaccaagaaagaaaagctgagttagctcctcatgtagaaaaatgtattacaatgcaattacaagatgcatttaataaaaggtcaaagaaaaaatttaaattcttttaaggagtaa |
| | gtgtggtagaaattattctttctcatctcatatttgatcaagcttatttttcaaaagtttggccatatatggattcagaatattttgaaagtggtccagctaaaaatacattcaaattaattaaatctcatgt |
| | taatgagtaccatagcgttccatctattaatgcgttaaatgttgcattagaaaatagttcatttactgaaacagaatattctggtgtaaaaacacttatttcaaaactagctgattctccggaagacc |
| | acagctggttagtaaaagaaacagaaaaatatgttcagcaaagggcgatgtttaatgctacgtctaaaataatcgaaattcaaactaatgctgagcttcctccggaaaaacgaaataagaaaa |
| | tgccggatgttggtgctattcctgacatcatgcgccaagcattatcaatttcatttgatagctacgttggtcatgattggatggatgactacgaagcacgttggctatcttatatgaataaagctcg |
| | taaggttccatttaaactcagaattctaaacaaaattactaaaggcggagctgagactggaacactgaacgttttaatggctggcgttaacgtcggtaagtcattaggattgtgttcattggcag |
| | cagattatttacagctcggacataatgttctttacatttcaatggaaatggcagaagaagtctgtgctaaacgtattgatgctaatatgcttgatgtttctcttgatgacattgatgatgggcatatttc |
| | ttacgctgagtataaaggaaaaatggaaaaatggcgtgagaaatctactctcggtcgtttaatcgttaaacagtatcctaccggtggagcagatgctaatacatttcgatcgcttttaaatgaatt |
| | gaagctcaaaaagaattttgttccaacaatcattattgtcgactatctaggtatttgtaaatcttgccgcattagagtttattcagaaaatagttacacaactgttaaagctattgcagaggaattgc |
| | gtgctctggctgttgaaaccgaaactgttctttggactgcagcacaggttggtaaacaagcttgggactcttccgatgttaacatgagcgatattgcagaatctgccggtcttccagcaacagc |
| | cgattttatgcttgcagtcattgaaaccgaggagctagcagctgctgaacaacaactcattaagcaaatcaaatcacgatatggtgataaaaacaaatggaataagtttttgatgggtgttcaaa |
| | aaggaaatcagaaatgggtagaaattgaacaagattctactccaactgaagtgaacgaagtagcaggttcacaacagattcaggctgagcagaatcgctatcaaagaaatgaatccactcg |
| | agctcagttagatgctttggcgaatgaattaaaattttag (SEQ ID NO: 422) |
| 11 | atgaaagaattttatatctctatcgaaacagtcggaaataatattattgaacgttatattgatgaaaacggaaaggaacgtactcgtgaagtagaatatcttccgactatgtttaggcattgtaagg |
| | aagagtcaaaatacaaagacatctatggtaaaaactgtgctcctcaaaaatttccatcaatgaaagatgctcgagattggatgaagcgaatggaagacatcggtctcgaagctctcggtatga |
| | acgattttaaactcgcttatatcagtgatacgtatggttcagaaattgtttatgaccgaaaatttgttcgtgtagctaactgtgacattgaggttactggtgataaatttcctgacccaatgaaagca |
| | gaatatgaaattgatgctatcactcattatgattcaattgacgaccgtttttatgttttcgaccttttgaattcaatgtacggttcagtatcaaaatgggatgcaaagttagctgctaagcttgactgtg |
| | aaggtggtgatgaagttcctcaagaaattcttgaccgagtaatttatatgccatttgataatgagcgtgatatgctcatggaatatattaatctctgggaacagaaacgacctgctatttttactggt |
| | tggaatattgaggggtttgacgttccgtatatcatgaatcgcgttaaaatgattctgggtgaacgcagtatgaaacgtttctctccaatcggtcgggtaaaatctaaactaattcaaaatatgtac |
| | ggtagcaaagaaatttattctattgatggcgtatctattcttgattatttagatttgtacaagaaattcgcttttactaatttgccgtcattctctttggaatcagttgctcaacatgaaaccaaaaaagg |
| | taaattaccatacgacggtcctattaataaacttcgtgagactaatcatcaacgatacattagttataacatcattgacgtagaatcagttcaagcaattgataaaattcgtgggtttatcgatctag |
| | ttttaagtatgtcttattatgctaaaatgcctttttctggtgtaatgagtcctattaaaacttgggatgctattatttttaactcattgaaaggtgaacacaaggttattcctcaacaaggttcgcacgtta |
| | aacagagttttccgggtgcatttgtatttgaacctaaaccaattgctcgtcgatacattatgagttttgacttgacgtctctgtatccgagcattattcgccaggttaacattagtcctgaaactattc |
| | gtggtcagtttaaagttcatccaattcatgaatatatcgcaggaacagctcctaaaccaagtgatgaatattcttgttctccgaatggatggatgtatgataagcatcaagaaggtatcattccaa |
| | aggaaatcgctaaagtatttttccagcgtaaagattggaaaaagaaaatgttcgctgaagaaatgaatgccgaagctattaaaaagattattatgaaaggcgcagggtcttgttcaactaaacc |
| | agaagttgaacgatatgttaagttcactgatgatttcttaaatgaactatcgaattatactgaatctgttcttaatagtctgattgaagaatgtgaaaaagcagctacacttgctaatacaaatcagc |
| | tgaaccgtaaaattcttattaacagtctttatggtgctcttggtaatattcatttccgttactatgatttacgaaatgctactgctatcacaatttttggtcaagttggtattcagtggattgctcgtaaaa |
| | ttaatgaatatctgaataaagtatgcggaactaatgatgaagatttcatcgcagcaggtgatactgattcggtatatgtttgtgtagataaagttattgaaaaagttggtcttgaccgattcaaaga |
| | gcagaacgatttggttgaattcatgaatcagtttggtaagaaaaagatggaacctatgattgatgttgcatatcgtgagttatgtgattatatgaataaccgcgagcatctgatgcatatggaccg |
| | tgaagctatttcttgccctccgcttggttcaaagggtgttggtggattttggaaagcgaaaaaacgttatgctctgaacgtttatgatatggaagataagcgatttgctgaaccgcatctaaaaat |
| | catgggtatggaaactcagcagagttcaacaccaaaagcagtgcaagaagcactcgaagaaagtattcgtcgtattcttcaggaaggcgaagagtctgtccaagaatattacaagaacttc |
| | gagaaagaatatcgtcaacttgactataaagttattgctgaagtaaaaactgcgaacgatatagcgaaatatgatgataaaggttggccaggatttaaatgtccgttccatattcgtggtgtgct |
| | aacttatcgtcgagctgttagtggtctgggtgtagctccaattttggatggaaataaagtaatggttcttccattacgtgaaggaaatccgtttggtgataagtgcattgcttggccatcgggtac |
| | agaacttccaaaagaaattcgttctgatgtactatcttggattgactactcaactttgttccaaaaatcgtttgttaaaccgcttgcgggtatgtgtgaatcggcaggtatggactatgaggaaaaa |
| | gcttcgttagacttcctgtttggctga (SEQ ID NO: 423) |
| 12 | atgaaactgtctaaagatactactgctctgcttaaaaatttcgctactattaactctggtattatgcttaaatccggtcaatttattatgactcgcgcagttaatggtacaacttatgcggaagcaaat |
| | atttctgacgttattgattttgatgtagcgatttacgatttgaacggttttctcggtattctgtctctagttaatgatgatgcagaaatttcccagtcagaagatggaaatattaaaattgctgatgctcg |
| | ttcaacaattttttggccagcagccgatccgagtacagtagttgctcctaataaaccaattccattcccggtagcatctgttgttactgaaattaaagctgaagaccttcaacaactgttgcgtgta |
| | tctcgtggtctgcaaattgatacaattgctatcacggtaaaagaaggtaaaatcgtaattaacggttttaataaagtagaagattctgctctgacccgtgttaaatattctttgactcttggtgattat |
| | gatggtgaaaatacatttaatttcattatcaatatggcaaatatgaaaatgcaaccaggaaattataaacttctgctctgggcaaaaggtaaacaaggtgctgctaaatttgaaggtgaacacgc |
| | gaattatgtagtagctcttgaagctgattctacccacgatttttaa (SEQ ID NO: 424) |
| 13 | atggaatattcaactggacagcatctattaactattcctgaaataaaacgatatattctgagaaataatttttctaatgaagagcatatagttactgaatctatgcttaggaatgcatttaaagcaga |
| | atatacaaaaataatgtccaatagaaatgaagcttggactgttactgattattatgactaaaggtgtattatgactaaaattactgtgaattatactgttgatgtaaaagatattcagccaaaacacg |
| | tgcgttctgaatcaaatccacaaaaccaaaataaaattcgtcgagcatgggttttgtctctttctgataacgcaatggaagttattcagaacaaaattaaatctgcacctgctcgtcatgcgtatta |
| | tgaagctatcgatcgtgaagtaagtaataaatggattgaactaatgcgcaaacatactacagaatccctaaacgccggtgctaaatttattatgacttcatgtggtgaacgccttgaagatgatt |
| | attgcggtaatgcagatgaacgtctaattgttgctgctcaaattgttgcggaaacaattgcggctgattttaatcgttaa (SEQ ID NO: 425) |
| 14 | atgaaagtatgtatttttatggctcgaggtcttgaaggttgcggtgtaactaaattttctcttgagcaacgtgattggtttattaaaaatggtcatgaagtaactttggtttatgctaaagataaatcatt |
| | tactcgtaactgtgcgcatgattataaatcattttcaattccggttttattggcaaaagaatacgataaaacacttaagctggtaaatgattgtgatattctaattatcaattcagttcctgctacttcag |
| | ttgaagaagacactattaataactataaaaaaattattgataacattaaaccttcggttcgtgttgtagtttatcaacatgaccattcttctctttctttgcgtcgaaatttgggattagaagaaactgtt |
| | cgtcgagctgatgttatttttagccattctgataatggtgattttaataaagttctgatgaaagaatggtatccagaaactgtttctctgtttgatgatattgaagaagcgccgacagtatataactttc |
| | agcctcctatggatattgcgaaggttcggtcaacctactggaaagatgtttctgaaattaacatgaatatcaaccgttggattggtcgtacgactacatggaaaggtttttatcagatgtttgatttt |
| | cacgaaaaacatcttaaacctgcaggactaagtactattatggaaggtctggaacgttctccagcgttcattcctattaaagaaaaaggaattccatacgagtattatcgtcttcatcaagtaga |
| | ccaaattaaaattgctcctaatttaccaacgcaaattcttgaccgttatgtaaatagcgaaatgcttgaacgcatgagtaaatccggatttggttatcagttgagtaagttggacaaaaaatatcta |
| | caacgttctttagaatatactcatctcgagcttggtgcatgtggaacaattcctgttttctggaaatcaacgggtgataatttaaaattccgtgttgataatactcctttgacctcgcatgatagcggt |
| | atcatttggtttgatgaaaatgacatggaatcaacattcgagcgtattaaagaactgtcatctgaccgaactctttatgaccgcgaacgtgaaaaagcatatgaatttttgtatcagcatcaagatt |
| | caagcttctgctttaaagaacagtttgacattattacaaaataa (SEQ ID NO: 426) |
| 15 | atgactattcaaattaaaaacgccatcaattcttacgcatatgataaagtagtttctttgttagaaaaaggcgatattgtaactcctcaaattttggataaatgggaaaaagagcttcatcagacga |
| | tgaaacagaatgatcagaagattggacgcaatactgtccgtgaattgttggttcaatatatcttgtcagaatttgatgttaaagcttttggtgtagaatctaaagcttatcaaaagcatgaaatttcc |
| | gataaaactattcgtcgcatgaaaaatcaacgcaagaaaaaatttgcagacctgaaaattactaaggtataattatgaacgaagctcttattaacgatttgcgtcttgctggatatgaagtaaata |
| | caaatggcattggtttaattcaaattgaaggaaacggattcatccttgagtatgaatttagccaatggtggttatacgctaattacggtgaattaattgaatatgttgaccaatttgattcactagatg |
| | cagctcttggagcggctaagctgatgaattcttga (SEQ ID NO: 427) |
| 16 | atgttattgactggcaaattatacaaagaagaaaaacaaaaattttataatgcacaaaacggtaaatgcttaatttgccaacgagaactaaatcctgatgttcaagctaatcacctcgaccatga |
| | ccatgaattaaatggaccaaaagcaggaaaggtgcgtggattgctttgtaatctctgcaatgccgcagaaggtcaaatgaagcacaaatttaatcgttctggcttaaagggacaaggggttg |
| | attatcttgaatggttagaaaatttacttacttacttaaaatccgattacacccaaaataatattcaccctaactttgttggagataaatcaaaggaattttctcgtttaggaaaagaggaaatgatgg |
| | ccgagatgcttcaaagaggatttgaatataatgaatctgacaccaaaacacagttaatagcttcattcaagaagcagcttagaaagagtttaaaatgacaattgaaaaagaaattgaaggattg |
| | attcataaaactaataaagaccttttaaacgagaatgctaataaagattctcgtgtttttccaactcaacgggaccttatggctggtattgtgtctaaacacattgccaaaaatatggtcccgtctttt |
| | attatgaaagcgcatgaaagcggaattattcatttccatgatattgattattcccctgctcttccatttactaattgctgtttagtagatttaaaaggaatgcttgaaaacggatttaagcttggtaatg |
| | cacagattgaaactcctaaatcaattggcgttgctactgcaattatggcacaaattactgcacaggttgcttctcaccaatacggcggaacgacttttgccaatgtagataaagtactttctcctta |
| | tgttaaacgcacatatgcaaaacatattgaggatgcagaaaaatggcaaatcgctgatgcgttgaattatgctcaatctaaaacagaaaaagacgtatacgatgcattccaagcttatgaatat |
| | gaagtaaatactctctttagttcaaacggacaaacgccttttgtaacaattacatttggtacgggaactgactggactgaacgaatgattcagaaagcaattctgaaaaatcgcattaaaggtctt |
| | ggccgtgatgggataactcctattttccctaagcttgttatgttcgttgaagaaggtgttaatctttataaagacgatccgaactatgatattaagcagcttgctttagagtgtgcaagcaaaagga |
| | tgtatcctgatattatttcagctaagaacaataaagctatcactggttcatctgttcctgtttctccaatgggttgccgtagtttcttgggcgtatggaaagattcgactggcaatgaaattcttgatg |
| | gacgtaataatcttggtgttgtaacactgaatcttcctcgcatcgcgttagattcttatattggaacacagttcaatgaacagaaatttgttgaattgttcaatgaacgaatggatttatgttttgaag |
| | ctttgatgtgtagaattagttccttaaaaggagttaaagctactgttgctcctattctttaccaagaaggtgcattcggggttcgtcttaaacctgatgacgacataattgagttatttaaaaacggta |
| | gaagttcagtgtctttaggatacattggtattcacgaattgaatattcttgtcggtcgtgatattggacgagaaattttaactaaaatgaatgctcatcttaaacagtggactgaaagaaccggattt |
| | gcttttagtttatattctactcctgctgaaaacctttgttatcgcttctgtaaactcgatacagaaaaatatggaagcgtaaaagatgttaccgataaaggctggtacactaacagtttccatgtttca |
| | gtagaagaaaatattactccgtttgaaaagatttctcgtgaagcgccatatcatttcattgcgacaggtggtcacatttcttatgttgaacttcctgatatgaaaaataacttaaagggtcttgaggc |
| | cgtatgggattatgctgcacaacatttagattattttggtgttaacatgccggtagataaatgttttacatgtggaagtacccatgaaatgactcctactgaaaacggatttgtttgttctatttgtgg |
| | agaaactgatcctaaaaagatgaacacaataagaagaacgtgtggttatttgggaaatccgaacgaacgtggatttaatctcggcaaaaataaagaaatcatgcatagggttaagcatcaat |
| | gaattatgatagattttatccttgcgattttgtgaatggccctggttgcagggtcgttcttttcgttacaggttgtttgcataaatgtgaagggtgttataataaatcaacatggaatgctagaaatgg |
| | tattccattcactggtgaaacactagaacaattaattgaatgtttgaataatgattatatagaaggattgactataactggaggagaccctctctatccggataatcgagatgtcattcattgcattg |
| | ttcaaacagtaaaaaatctttatcccaataaaagcatttggttgtggacaggatataagtttgaagatattaaacaactagaaatgcttaaatatgttgatgttattattgatgggaagtatgagaaa |
| | aatcttccgactaaaaagctgtggcgaggatcagataatcagcgactttggtcaaataccgatggggtgtggaaacatgattaaattgaattacattatggatactataaatgatatgatttttcat |
| | tttggtccagaattttattcccaatatagtttagtgcttatcaatgcttggttaattaattaagggtaaaatatgtataaatttcgtaaaggtttagctgattttcttacaactgtaacattctttctgtttatg |
| | gcagttggagctattttccttattccttttattgctatatttttcgtgattagtttaatttctccagaaaagggcttatcttccagtgagttcaatgagcgcctggataaaattactaacaagctgaatgct |
| | gctcttagtaaggaatagttgtgaaacaaaataagattgaagtctatggaattccagatgaagtaggtcgttgtcctggatgtcaatcagttacaaaacttctaaaggagctcaatgctcctttta |
| | ctttctataaagttcttacaaataatggtaagattgagtatgatcgtccactgattgtatctcttgctaaacgcgctggattcacatctcttaacattcgttatccagtcattttcattaatgattctagac |
| | aaaagaacattaaacacttcaaagaaactctcatttcacttggatatgatagagatatcatagaagattaa (SEQ ID NO: 428) |
| 18 | atgaaacagttgataattaaaagattgaatttattgatatgttgtttatgtatagtaattgcatatggttattacgcaattaatgattatatgcattataaagattatgatgttactgtagttaataccctta |
| | caggaactcaaggaaaggggtctagtttatcgtttattgccgtatatgaactcaaagacggttatagatttagcgaatatatttcgccagagatgtattcatcaatagaaaaaggcgataattact |
| | gtaagtttacgtcctttcgacgtaaaacagacattgtttgataatattgtttggttctttggaatggtattagttcaatctatatgtggtacttatatagtctgttcaatcttattccgcgtaattagtaaaa |
| | ttgagtgaggaaaatatgtcagtagtaattaataatgtcaatgcagtaattaaatctttagttaataaaaaaatgatgaatgaatggactgtacttcgtcgtggagagccagataaattttttcatag |
| | atttaacccaactttggatttgaatgttattgacagagatgttcatgctgaaattttagataaatttaaagttgatattggatttggattagaaaaacatttacagcgaacaaacgggtctggaatga |
| | gtttatctaatcgcatcatgaaagcccttaataaaattggagcattgtctcgtattaacgcgagtgaaatccttcgtaattataataaaggatatgacctttatggccgactaatgccgaaattatca |
| | ttcgatcaaatgattgcggatttgtgggaaaatcaacgacgattattagcattaggcgctcgattagctaaaggtctagataaacaaatgatttttaaaactaataatacagaagaccttaaatgc |
| | tttaaatttagtactcgtggagatgattattacgtcagagctcgctctacagattatgtcaatatggggcatcatctctgtttagcttttgaagttttaaaagaagctTgaacgttagaatattcatct |
| | ggtgctaaatgcccgattggttcaaattgcattttaatttatcgcccgaatgaatccagttcaactaaattgcctacaaaacctgtaccagttcgtagtaacgaaaaacattctgaacaaattgatt |
| | attttaataaacagattgaagagctgaatatttctattcaacaatatgacgatgaaatctttagactatctggattgagtagtaaagctaaatctgaacgtgaaaaattaattaaaattgttgatttact |
| | taaatcttaaggaacaccatgaaaactcgttctcaaattgaagatatggttcgtaatgccagctatactcgtgatgctatgacatttttgtgtgaaaataatttagaccttaataaagttaattgttcc |
| | attcacgcctttaaacatctgaacagcagtgaatgggtgcgtaattttaatgaagcagggtatattacacaaatgactgctcgtgagcagctcgttgatttctgtaaaactattgattataaaaatc |
| | ctctatttgttcaaggcgttggtcagagtaaggttgatttatcaacaggattttttaatccaaatcattatcgtcttgaatggagatttattgctctattccgtaaacaattaaagcaaattttgtcgact |
| | gctagtcgattaaaaggttctgatattaacttaaagaatctgaaatttgatggttatactcttcagatggaagtaagaccattaaaagaaaataatagaactgcacgaattagctttaaacctaata |
| | caaaaaattctctttcaatttgtgaatgccttaaatcacagttgatagaagcatttaagtatatggatgttgttgctagtgttcagtctaagatttcacagcatttcgaacgatttaaattaggcacaac |
| | aacgtatgaacttgatatggtcgttttatttaaatacgattttttgagaaaggacgaagttgtacaagagaaaaagcaggaagtgcaagataacttaaatttatctaattacttatcaaacgatccta |
| | aattttggatgtatagttcaggtaataaagatgcatggaaattcaataaagtgaattttcttcctattgaaaatccgagtcttaaacctgttgaaaaatggcacgcggatgcgattgagaagtctat |
| | caaggcagtagatgatgaactcgttaaagcaactaatgaagtgttagaagctgaaaagatgctagaaaaagcacaagaaaaagtcaaaaatctcacgaagcaacgttctaaactgaacaat |
| | gcactaaatgcactgaactag (SEQ ID NO: 429) |
| 19 | atgaacgctaaagatattttcaacctggtaaattacaacgatggtaaatttaaatctgaagcacaaagcaagttctttaatgacatctcaatcggaggtgaaatcacagttgatggaggacaaat |
| | ttacaaatcccgttggaattggatcgttattatcgatgagattggtattgtagaaatttacaaaaatacgaataaaaatcgtacattacactggtctcgtgatactaacgaacagtacaaaaagga |
| | taaagcatctaagttatctcgtgtaactcaagaagatattgagttcatcaagaaagatattttgatgtatgataacttaattgctgaagagcaagctgttattgataaatttgacgagattaaagcttc |
| | tcgtgaaattcctgattttatgaaagaatcagtaaatgaacgatacactctcatttcagagcgtattgaaacttacaaaaagcaaagagctgaacgccaaaatactcttcggaagtttgaagaac |
| | ggttaaagacggtactcgcataa (SEQ ID NO: 430) |
| 20 | atgttatactcaaaggctcgtgaaatttacgaaactaagattaaagaagctgtatttcaattcgcaacaacgatgcgatggacaaatgattgggaatattcaaaaaatcataagaagcccctgg |
| | tgacaagaaaggctcatatgttagtgttaatagaccgtgagcagattaaagcccgagaagccctccagaatcataaaaaggctgcctttgaatggtttatggataacactgctcctgagacta |
| | agaaagcagtgagcgcgtggttcagtggaaaaaattgtgaaagaagtttcttttag (SEQ ID NO: 431) |
| 21 | atgaaagttttgtttgttgtgtatgtgatgattcaatataattacccaatgtttacttataatttggtgaataacattattgatatgattcagaggagtatgtaattatgagtgagtcgaagagaatcaat |
| | atgaaacgattagtattagaagatagtgtgctttttggtgaattagcgatcgaaaaagtaaataacatgtatcgtttgacgcaagaagatgatatgttatattacgcctagtgaaattgttcgtttaa |
| | cccaaattgaatatgcttacactgataaaattgtaagcattaatgatgagcataaaattcatttttattcttcatgcccaggatttaatattaaaagcgagtcaatgtgcttatcaattaataattggga |
| | taattttataactaacattaaatatttttatgattctactaaaagaaaacataatttaaaatggtttaaaaatgtaatgctattattactaactcctgtaatcagaatgatgaaactattttaaatgtttcaaa |
| | atgctatgaagagggagatgtagtatctattcgtcaaattgacgattttcgatcgcatatcattacattaaacaaagacgaagctattgcactaaagacttatcttgattctgttattccaactatgat |
| | ttcaaagtgaggaaatatgtttatttcaagtggaagtggtttaattcgtgttgaatttaaaaatgacatcttccttagtcaaggagatgatattattaaaatgagttatgacgaaatcaagaaaatttg |
| | tcatactcttgaaagccgtggaaaagtaaatgctgttttgacattggtgatttatgggtaacgctttatgaagtatccgaaggatttaacattgaagatgaaaataacattttagctattgataaaag |
| | aactgatttgcttgatgtattaaaagcctatgaacagtcaaacggtggaagaaaagctgtattgatttatcaaaaaccgcattcatgtggaactgcttcaatcatttcaaatattgaaggcgaagtt |
| | gatacttatatgtgttttaaaagctggtggtgaccgtcatccggattttatttctattcgtcaaaacaatggagaaatttcattatcaaaatcagaagctgaagctatgattaagtatttaacaaccgt |
| | tacgccttcaatgaaaggataattatgattattaatgaaaactcttggcactataaattattcaaactgtttaacgatgaatggcaacgacctaagacactatgcgcatatttttggtctattgcctcc |
| | tacatttttcgtttctatttttgggtgtgctatactcgtagggctaacaattatttgtgcagaaagcctacaacgttggcttattttcggtagtttatggactcttcttccatcggcatttatacttgcgcttt |
| | tggttgttttacttattatcggttcatttgttattcctgcacatttgcgtgaaaaatataaagattataaatggaaaaaggattatgctttacacgtagaaaatattgatagggcgtataaaggtttacct |
| | cctattcaacctaagaaatcgattatcgttgaatttttaaaagttcgtaaagctaaagtatgtcctgttattgaatataaggctgaatgatgaaaacagtaatgaaaagctattttggtagtcatcttta |
| | tggaacttctaccccagaatctgatgtagattttaaagaaatttttgttcctcctgctcgcgatattcttatcggaaatgtcaaagagcacatgagcaaaaacactaacaacacatcatctaaaaa |
| | cactaaagatgatattgaccatgaactatacagtcttaaatatttctttaaattagcagcagatggtgaaactgtagcgttagatatgcttcacactcaacctgaactagtggttaaatctgatttgc |
| | ctgatgtgtggaagtttattcaagacaaccgttctcgtttttatacgactaacatgaaatcatatttaggatatgtccgtaagcaagcttctaaatacggtgtcaagggttctcgtttggctgcattac |
| | gtgatgtattgaaagtagttaatcaaatccccgagcaatgggttgattaccaagaagatggttctattaagcagcgtcgtactaaagttgaagatattaagcatcgtcttccagaaaacgaattct |
| | gtgaatgggtgttccataatcatgagaaaacaggcccacaaacgttctacactgtattgggtcgtaaatatcagacaacgctttctcttattgagcttaagcagtcactgaacaaattagatgct |
| | gaatatggtgaacgtgcccgtaaggccgaagccaacgaaggcattgactggaaagctctgagccatgcttgtcgtggtggacttcaactattggaaatttacaaaactggtgacttggtttat |
| | ccacttcaagacgctccatttattctcgacgtgaagttgggtaaacatccatttaaaacggttcaagagtttttggaagatgtggtcgatcaagtagaagcagcatctactgaagcttctaagaa |
| | cggtatgcagcaaaaagtagacatgggtttctgggatgacttccttgagaaggtttatcttgaaaaccaccgaagttattataaatga (SEQ ID NO: 432) |
| 22 | atgctacaattaactgaaaagcaacttcgcaatcttactgtgcttcaattagatgaaattcgtagggaagttggaaatatcatttcagctttgcgtcgagaagtatcacttaaccaatctccggca |
| | gactatactagattgcgaaattttgaaaaataccttgataaagttaaggccgtgcatcggcataaagtaaatacaggacaaaaatgataggaggcctttatggccttaaaagcaacggtactat |
| | ttgccatgctaggattgtcatttgttttatctccatcaattgaagcgaatgtcgatcctcattttgataaatttatggaatctggtattaggcacgtttatacactttttgaaaataaaagcgtagaatcg |
| | tctgaacaattctatagttttatgagaacgacctataaaaatgacccgtgctcttctgattttgaatgtatagagcgaggcgcggagatggcacaatcatacgctagaattatgaaaattaaattg |
| | gagactgaatgaaattcagcgacttttcacaaagtggaaaaccttcaaaggcagatgaatacttaggtttattaatggctgcacaagcttattttcattctgcacattttgaaactaaaagttatgct |
| | agacacaaagcatacgattttattttttccgagttgccagatttgattgataaattttgtgagcaatatttggggtattctggtagaaaatacacaccttcaattccagatgccagtaaacttcctacc |
| | gacacaattaaaatgattgatcgcatactagaccaatctaacagcatttataaagaaatgcctccagccatccaaagcacgatagatgatattactggaatgttttaccagagtaagtatcttcttt |
| | ccctcgaataa (SEQ ID NO: 433) |
| 23 | atgaaaacctatcaagaatttattactgaagcagctattaattctcaaattattgctgaatcttttactgatcttttgaaatttaaaaaaggtcagaaaatcactgctgtattggatgatggtacagaa |
| | gttgagatggatgtacagggatataattatgcagtagatggaaaactgtataataaatctcatgctaaatttgattcatttgacgactttgttaatacagttgaagatgaaaaaactcgtcgatccat |
| | tgcaactggtgatgctaaggttcttatggcacatggtcatgaacgcattcgcgctaaacagaataaaatgggtgaagataatttcgcattagttggttatcaatctggtaaacaaacttatggcta |
| | tcaacgtactgctaccatgtataacaaaaatggtaaaattgcctttgtgaatagtaaaggttctattcagtacgttaaatcgttcaaataacatgggaacaacctggacctcatgattctgtgagg |
| | gattcccgccaacctgtaataatgtcgagcccaagcgcggtaatgggtaaatacagaaatggacaattcatgcgccatggaatggcccaaatttagagagaagaaatgagaacatttttaac |
| | tggtccttatctatccctgatgaatgcttttacacaccattctgatgctagagtagaagaaatttgtaaaaacgaatatcccgccatttgaagacttacttaaacagtattgcacacttcgactagat |
| | ggtggacgtcaatctggtaaatcaattgctgtgactaactttgctgctaattggttgtatgatggcggaacagttattgttctttctaatacttcagcttacgctaaaatttctgcaaataacatcaaa |
| | aaggaattttcgcgttattctaatgatgatatacgttttcgtttatttactgattctgtgcgcagttattggtaataaaggaagcaagttcagaggtttatcgctttcgcgaattttgtatataattgatga |
| | gcctgtcaaatctcctgatatggataagatttatagtgtccatattgacactgtacactgctgctgtaatattaaatgttgtattggtggtattactcgtccacagtttttcgtaatcggaatgcaatga |
| | tgacagacactcagcttttcgaatatctttatttttcgccaaaaactattaaaaataaattggtgaatcattttgaaattttggcaaaaaataacattttaagcgaattttatcctaagcaatacaaatta |
| | caaaaaggcgtattcaaaggatgcagagttttgtgtactgctcctaatgcacggctaatgaataaaattccatattttaccatggaatttattgatggaccttttaaaggattaattacccacagttt |
| | aatggcatatgattctgagccatttttaattaaagaacaatcttggataaatttattttctaattgaggtttatatgaaagcatatcaaattcttgaaggcacacataaaggtactatttattttgaagat |
| | ggtattcaagcacgaattattgtctctaaaacctttaaagaggactcttttgtagacccagaaattttctatggtttgcatgcccgtgaaattgaaattgagccacaacctacagttaaaattgaag |
| | gtggtcaacacctgaacgttaacgttctgcgtcgtgaaactctggaagatgcagttaagcatccggaaaaatatccgcagctgaccatccgtgtatccggttatgcagttcgctttaactctctg |
| | actccggaacagcagcgcgacgttatcgctcgtacctttaccgagagtttgtaatggcaaagataattattgaaggttctaaagatgtgataaatgctttcgccgagtggtttagtaattcaggc |
| | gaacagcaatttaatgaagcctggaatatgggtgatattgatggaatttatcctacgacagaagtttctgttcagggatatggcattcatgaacctattcgtttagttgaatatgatttatgtactggt |
| | gaggaagtcaaatatgattgaagatattaagggataaaccacatactgaagagaaaatcggtaaagtgaatgctatcaaagacgctgaagttcgtttaggacttatctttgatgctttatatgat |
| | gaattctgggaagcactagataattgtgaagactgtgaattcgcgaagaattatgctgaaagcctcgatcagttaactattgctaaaacgaaactcaaagaagccagtatgtgggcttgtcgtg |
| | cagtgttccaaccagaggaaaaatactaatggatcaattaagcgcagggtttggttatgagtattatactgcacctcgtcgtgtatctgttgctcctaagaaaattcaaagtcttgatgacttcca |
| | ggaagtagtccgtaacgctttccaggactatgcacggtatcttaaagaagattcgcaggactgtctcgaagaagatgaaattgcttactatacgcagcgtcttgaacagctcaaaaatctacat |
| | gaggttcgtgcagaagtttcaaagtctatgaataaattgattagatttaaagaataa (SEQ ID NO: 434) |
| 24 | atgaatatatttgaaatgttgcgtatagatgaaggtcttagacttaaaatctataaagacacagaaggctattacactattggcatcggtcatttgcttacaaaaagtccatcacttagtgttgctaa |
| | atctgaattagataaagctattggacgtaattgcaatggtgtaattaccaaagacgaggctgaaaaactctttaatcaggatgttgatgctgctgttcgcggaattctgagaaatgctaaattaaa |
| | accggtttatgattctcttgatgcggttcgtcgttgtgcattgattaatatggtcttccaaatgggagaaaccggtgtggcaggatttactaactctttacgtatgcttcaacaaaaacgctgggat |
| | gaagcagcagttaacttagctaaaagtagatggtataatcaaacacctaatcgcgcaaaacgagtcattgcaacgtttagaactggcacttgtgacgcgtataaaaatctataa (SEQ ID |
| | NO: 435) |
| 25 | atgaacacactgaagaaaattgttgagtttattcgcactaaacttggttctgctatggctaaaaatctatctgttgaagaacagtatactgccgcagcagcaaaactgcttgataaaattaaagac |
| | ctaaaaactgcttctgttaaatctattaatgaagaaaaacgtattcgtgaacttattgttgaaaagaataaacaggctgaatcaaaagagcgtgaaattcgcaagcttctttccgaaggtcaagat |
| | gtaacaatgcatgctaaactcggtttgctatatcgtcgaacagctgaacagctgactactaaagctgatggttatgctgaaatgcgaattgaaatcgctaagaaagtagttgagttagatgatg |
| | ctcgccaagaacttgcagttaaattggaatatatccgtgaaactcgtgcagcaaatgcccttggaattagtactgctgatgatgtagttgaaattgcagcactgactaaggttgatattgaagat |
| | actcttgctcgagttgaaacctttaatggcaatatttctggggttgaaactacctctgccgatgttcaggaatatattaattctctgaaataa (SEQ ID NO: 436) |
| 26 | atgactactttaattatttggttcgacgaaaatgaagaaacatattgcgtgaacattggcgaaagcccaatgccagaatttgaatcttcagataaaaactcggttgtatcttgggctgaaggttat |
| | aaagcagcaaaaggcgatgttgaaatagtttacaaactatccggagtataa (SEQ ID NO: 437) |
| 27 | atggataattacggtgaactgttcaacttctttatgaaatgtgtttcagaagatttcggtcgtacagtgaatgatattaaagttatcggtcctgaccatccgatgtttgaaacttacgcagtaatggg |
| | taatgaagatggtcagtggtatactgtaaaggtcgtgattaacatgttcactgctgaaggttatgttaaactgtcttctaaagtttaccatgataacgacgaaatcgcagaagaatatttcaataat |
| | atgaaataa (SEQ ID NO: 438) |
| 28 | atgaaaggtaatgtttatttagtcgttcatgatttaacattctattttaatcataatgacactgttatttctgaacgtgtaattaatttgctttatcagcatgcagactatgtttatgtcgaaaacgaattta |
| | ggcattggcaatttctcaaaaatcgttcatttggtttagatggttacgaatactttgaacgtaaagaccttttagataaaattccattatctacacaataccaaaatcacaagtctttacataaatgcc |
| | ggctaattcgaaatgctgaatccgcgtatgaagcaattgatttatggcgtaaacgccgtgaacagattgatgctttaaaagaatattaa (SEQ ID NO: 439) |
| 29 | atgaatggctattggtggaaatcaacgggaaaatatgataagcgtggaagaaagggtcatgaatactgcatgtgccgtttcggtgataaaggaccatattcattaaataacatatattgcgcaa |
| | ctaataatcaaaatacaaaagatgcgagactaaatgatagatttcctccaaaatctaaaaattttaattttaatggtcgaaaacactcggcacagtccttagaaaaaatttctaaaaataatgcaa |
| | gtaccttaagcaaagatgagataactagacgattaaaaatattagaaaattttaatatggatgaacgaggttttattaaaaattatgcaaacgctataaatgttagccatactcaagctagaaagtt |
| | tttaaataaatattacataaaataa (SEQ ID NO: 440) |
| 30 | atgaaacgttgtgaattaattcgaaatgttgctattgcaatttctgcttccgcttttagtttttcaatgtttgttggatttatatgcggattattgactacagcagaaaatgtgttttcacttgtagtagcatt |
| | tttaattggtttaatcgctatcgttatggataaaatttctaaaggttaataatgattctttatgcgaaagtatcgtccgttgaaaatggatataaatatgatcaagatgcggctaaagccttgattgatg |
| | attatggcattttaacatgttttgaagttgaaaaggtttacattgaccgttcatcttctcaagttaaattagtgaaggaagaccgtaaatttaatacagtaaattttgatttctttattgaaacagaaaaa |
| | ggtcctcttgaatatgatattttcaagaatcctttgggtcttgaatgtattaaatatacttacattaatatggtgaacaaatgtatattcgtttaggcagcacaattcctaagggttacgtaattgatgtc |
| | actacctgggaaaatgatggtgataactataaaaccaaaacactgtttggcgtagaagagcatgagctccaacaatttaaatatcttttgaagaagtttaagagtcgtcattctagcactaaagc |
| | tgaccgttattgtggtaatgggttgttcagcgagcaagagctttttatatatgaatatttggttgaaggactgttctcagaccaactttatccagaattcattaaaaaggtctttgatatagaagttga |
| | ccttggtaataaatccgaagaagatgaagaacgtgtatttgacttattctttgtgaatggtaataagatatttgaaggcctcattgatattcttggtcatgcttctgaatactatgaatatgatttcttgc |
| | gtgtagttgaacatgtagaatttgcttatatcgaagaagaaattgttttgccgactgttaaaatggttgatttgctttaa (SEQ ID NO: 441) |
| 31 | atgaaaacatttaaagaatttatcaatgaagcggctgcgccaaagacattcgttattaatactcagacgagtcttgacgatgagtatgcagaggcaattctgaagtcacttgctaagaacggcg |
| | ttgaagtaatcgcctcggactttaagaaaggggcttccgagatgtttatttctataactaaaggatctaaagctaagatcaaatcatcattcggagttgctcgtaccgatcaaatcgacaatcatg |
| | actttaaacaaactggtgctaaacggcagaatacaattgcatcacgcggaataaaatag (SEQ ID NO: 442) |
| 32 | atgaaaactttcaaagagtttgctacaaaaactactattactgaatcttcccatggtatggaagtaaaacttggaatggctttagctgaagctgagcgtcttttctctcgtattaaagaacttgctgc |
| | tgttgatccttcatcttttaaaggagaccaaactaaagttaaagcgcttttagcattatgctctgatgcaggcgaaatcgctaagaacggttctaagatgaagaaacgattagaagatttaaaata |
| | a (SEQ ID NO: 443) |
| 33 | atgaaactaatctttttaagtggtgtaaagcgtagtggaaaagatactactgctgattttatcatgagcaattattctgcagttaaataccaacttgctggtcctattaaggatgcattggcttatgca |
| | tggggagtatttgcagcaaacactgactatccttgcttaactcgtaaagagtttgaaggaattgactatgatcgtgagactaatttaaatctgactaaattagaagtaatcacgattatggaacaa |
| | gcattttgctatcttaatggtaaaagcccaattaaaggtgtgtttgtttttgatgacgaaggaaaagaatcagttaatttcgtagcatttaacaagattactgacgttataaataatattgaagatcaa |
| | tggtcagtccgtcgtctgatgcaagccctaggtacggatttgattgttaataacttcgaccgcatgtactgggtaaaattatttgctttagattatcttgataaatttaactcaggttatgattattatat |
| | cgttcctgatacccgtcaagatcatgaaatggatgcggctagggcgatgggtgctacagtaattcatgtagttcgtcctggtcaaaaatccaatgatacacatattacagaagctggattgcca |
| | attcgtgatggcgatttagtaattacaaacgatggttctattgaagaacttttttctaaaattaaaaatacactaaaggtactataatgtctgaacaaactattgaacaaaaactgtctgctgaaatc |
| | gtaactctgaaatctcgcattcttgatacacaggaccaagcggctcgtctgatggaagaatccaaaattctgcaaggaactttggctgaaattgctcgtgcagtaggtatcactggcgatacc |
| | atcaaagttgaagaaatcgttgaagctgtcaagaatcttactgctgaatctgcagatgaagcaaaagatgaagaataatggaatttaaagacttttcaacgggtctttatgtagcagctaagtttt |
| | cagaattaacacttgatgcgctggaagaactccagcgctctttacgtgttcctaatccagttcctagagaaaaaattcattcgactatatgttattcaagagtaaatgttccatatgttccatcgagt |
| | ggaagttttgaagtagcttcttctggacatttagaagtatggaaaacacaagatggatcgactcttgtacttgtgctagattctgaatatctgcgctgtcgacacatgtatgcgcgggcattaggt |
| | gctacacatgattttgatgattacacaccgcatataacattgtcttataatgttgggcccctatcatttagcggtgatgtacaaattccggtcgtattagatcgtgaatacaaagagcctcttaaact |
| | cgattgggcagatgatttaaaataa (SEQ ID NO: 444) |
| 34 | atggcatattctggaaaatgggttcctaaaaatatatcaaagtatagaggtgaccctaaaaaaattacgtatagatcaaattgggaaaaattcttttttgaatggttagataaaaatccagaaatta |
| | ttgcatggggtagtgaaacagcagtaattccttatttttgtaatgcagaagggaaaaaacgtagatacttcatggatatttggatgaaagattcttctgggcaagaattttttattgaaataaaacct |
| | aaaaaagaaacacaaccaccggttaaaccagcacatctaacaaccgcagcgaagaaaagatttatgaatgaaatttatacatattctgttaataccgacaaatggaaagcagcacaatcttta |
| | gctgaaaagcgtggaataaaatttagaattctaacagaagatggattacgagctcttggctttaagggggcataatggctatttttcaaataattaatgaaagcactccccaagttccaaaggtt |
| | aagcaatcattaaacgaaaagaaatggattcagataggtcttgaatacaaaaaggccaaagcaaaaggaatgacaggaaagcaatttgctgaagaaagaggaatcaaatactctacgttta |
| | cttcagcaatgtcaaaatatgcttcaggaattaaaacggctgaaaagattcaaaaacttgaatcaaaaccaatgaataaactcaataagcaagaaagacaactgcttatgataaattcattcag |
| | acaaacattgcgtgataaaattcgtaatgaaggtgcagcaattaataataaaaccagaaagtggtttgccgaaactattaagcaagtaaaaggacataaagttgttcgcccgcagccgggac |
| | gaatatatgcttttgcttatgatgctaaacacaaggaaactcttccttattgggataaatttcctttgataatttaccttggtttaggtaagcataatttaatgtacggattgaacttgcactatattccac |
| | ctaaagctcgtcagcaatttctagaagagcttttaaagcaatatgcaaatacacctactattactaataaaacgaaattaaaaattgattggagtcaagtgaaaggatttagaggtgcagatcaa |
| | atgattaaggcgtatatacctggtaatattatgggtagccttgttgaaatcgccccgaaagactgggcgaacgttgtgttgatgccacttcagcagttcgtttcaaaaggaaaacgtttctctgc |
| | aaacaaagtctggtcaaatatctaa (SEQ ID NO: 445) |
| 35 | atgttcattcaagaaccaaagaaattgattgataccggcgaaattggtaacgcttctactggtgatatcttattcgacggtggtaataaaattaatagtgattttaacgcaatttataatgcgtttgg |
| | cgatcagcgtaaaatggcagtagcaaatggcactggagcagatggtcaaattatccatgctactggatattatcaaaaacactctattacagagtacgcaactccagtaaaagttggcactag |
| | gcatgatattgatacctctactgtaggtgttaaagttatcattgaaagaggcgaacttggcgactgcgttgaatttattaactctaatggatcaatatcagttactaatcctctaacaattcaagctat |
| | tgattcaattaaaggtgtttcaggtaatttagtagtaactagcccatatagtaaagttactttacgctgtatttcatctgataattctacatcggtttggaattattctattgaaagtatgtttggacaaaa |
| | ggaatcaccagctgaaggtacatggaatgtttctacatccggatcagttgatattccactatttcaacgcactgaatacaatatggctaaattgctagttacgtgccaatcagtagatggaagaa |
| | aaattaaaacagcagaaataaatattcttgtggatactgttaattcagaggtaatttcttctgaatatgctgtcatgcgagttgggaatgaaaccgaagaagatgaaatcgctaatattgcatttag |
| | tattaaagaaaactatgtaacggcgactataagttcttcaactgtcggtatgagagcagcagttaaagttatcgctacgcagaaaatcggggtggctcaataatgaaacaaaatattaatatcg |
| | gtaatgttgtagatgatggtaccggtgactacctgcgtaaaggtggtataaaaataaatgaaaactttgatgagctttattatgaactcggtgatggtgatgttccatattcagccggtgcctgga |
| | aaacttataatgcttcatcaggacaaacattaacagcagaatggggaaaatcatacgctattaatacatcttctggaagagtgactataaatcttccaaagggtacagttaatgattacaacaag |
| | gtaattagagctagagacgtatttgctacatggaacgtcaacccagttacactagtagctgcttccggcgatacgattaaagggtctgcagtaccagttgaaattaatgttcaattcagcgattt |
| | agaactagtgtattgtgccccaggacgttgggaatatgtcaaaaataaacaaattgacaaaattaccagttcagacattagtaatgtagctcgtaaagaatttttagtcgaagtccaagggcaa |
| | acagactttttagatgttttcagtggaactagttataatgtaaataacatcagagtaaaacatcgtggtaacgaattatattatggcgatgtgtttagcgaaaacagcgattttggctctccaggcg |
| | aaaatgaaggagaactggttcctcttgatggatttaatattcgattaagacagccttgtaatattggtgacactgttcaaattgaaacatttatggatggtgtatcgcagtggagaagttcatatac |
| | aagacgtcaaattagattgttagattcaaaattaacgtcaaaaacttctctagaaggaagtatttacgttactgatttatcaacaatgaaatcaattccattttctgcttttggattaattccaggagaa |
| | cctattaatcctaattctcttgaagttagttttaatggaattttacaagaattggctggaacagttggaatgccattatttcattgtgttggtgccgattcagacgatgaagtagaatgctctgttttag |
| | gtggaacttgggaacaatctcataccgattattcagttgaaactgatgaaaacggcataccagaaattttacatttcgatagagtatttgagcatggtgacattatcaatatcacctggtttaataa |
| | tgatttgggtacattattaacaaaagatgagattattgatgaaactgataatctctatgtatcgcaaggaccgggagtagatatttccggtgatgtaaatttaacagactttgataaaattggttggc |
| | caaatgtagaagcagttcaatcttatcaacgcgaatttactgctgtttcaaatatctttgatacgatttatcctattggaactatatatgaaaacgctgttaatccaaataaccctgttacatatatggg |
| | attcggctcatggaaattatttgggcaaggaaaagttttagttggatggaatgaagatatttcggaccctaactttgctctaaataacaacgatttagattctggtggaaatccttcgcatactgca |
| | ggcggaacaggtggttctacttctgttacattggaaaatgctaatcttcctgcaaccgagacagatgaagaagttctaatagttgatgaaaatggatcagtcattgttggtggatgtcaatacga |
| | tccagatgaatccggtccaatttatactaaataccgtgaagctaaagcatctactaactctactcacactccgccaacatcaataactaacattcaaccatatattacagtttatcgttggataagg |
| | attgcataatgagtttacttaataacaaagcgggagttatttcccgcttagccgattttcttggttttagacctaaaactggcgacattgatgtaatgaatcgtcaatcagtcgggtcagtgacaat |
| | atctcaattagcgaaaggattttatgaaccaaacatagaatcagctattaatgacgttcataatttttctataaaagacgttggtacaattattactaataaaactggtgtttctcctgagggtgtttct |
| | caaactgattattgggcattttctggaactgtaacagacgattctcttcctccgggttctcctgttacggtattagtatttggtcttccagtttcagcaacaactggaatgacggcaattgagtttgtt |
| | gcaaaagttcgtgttgcccttcaagaagctattgcatcatttactgctatcaactcatataaagaccatccaacagatggtagtaaattagaagttacttatttagataatcaaaaacatgtattaag |
| | cacatattctacatatggaataactatttcgcaggaaattatttctgagtctaaacctggctatggtacatggaatttattaggcgcacaaactgtaactttagataatcagcagactcctacagtat |
| | tttatcattttgagagaacagcatgagtaataatacatatcaacacgtttctaatgaatctcgttatgtaaaatttgatcctaccgatacgaattttccaccagagattactgatgttcaggctgctat |
| | agcagccatttctcctgctggcgtaaatggagttcctgatgcatcgtcaacaacaaagggaattttatttcttgccactgaacaggaagttatcgatggaactaataataccaaagcagttacac |
| | cagcaacgttggcaacaagattatcatatccaaacgcaactgaagctgtttacggattaacaagatattcaaccgatgatgaagccattgccggagttaataatgaatcttctataactccagct |
| | aaatttactgttgctcttaataatgtctttgaaactcgtgtttcaactgaatcatcaaatggggttattaaaatttcatctttaccgcaagcattggcaggtgcagatgatactactgcaatgactccat |
| | taaaaacacaacaattagctgttaaattgattgcgcaaattgctccttctaaaaatgctgctacagaatctgagcaaggtgtaattcagttagctacagtagcacaggctcgtcagggaacttta |
| | agagaaggatacgcaatttctccttatacgtttatgaattctactgctactgaagaatataaaggcgtaattaaattaggaacgcaatcagaagttaactcgaataatgcttctgttgcggttactg |
| | gagcaactcttaatggtcgtggttctacgacgtcaatgagaggcgtagttaaattaactacaaccgccggttcacagagtggaggcgatgcttcatcagccttagcttggaatgctgacgttat |
| | ccaccaaagaggcggtcaaactattaatggaacacttcgcattaataatacgcttacaatagcttcaggtggggcaaatattaccggaacagttaacatgactggcggttatattcaaggtaa |
| | acgcgtcgtaacacaaaatgaaattgatagaactattcctgtcggagctattatgatgtgggccgctgatagtcttcctagtgatgcttggcgtttttgccacggtggaactgtttcagcgtcaga |
| | ttgtccattatatgcttctagaattggaacaagatatggcggaagctcatcaaatcctggattgcctgacatgcgcggtctttttgttcgtggctctggccgtggctctcatttaacaaatccaaat |
| | gttaatggtaatgaccaatttggtaaacctagattaggtgtaggttgtactggtggatatgttggtgaagtacagaaacaacagatgtcttatcataaacatgctggtggatttggtgagtatgat |
| | gattctggggcattcggtaatactcgtagatcaaattttgttggtacacgtaaaggacttgactgggataaccgttcatacttcactaatgacgggtatgaaattgacccagcatcacaacgaaa |
| | ttccagatatacattaaatcgtcctgaattaattggaaatgaaacacgtccatggaacatttctttaaactacataattaaggtaaaagaatgacagatattgtactgaatgacttaccattcgttga |
| | cggccctcctgcagagggccagagccgcatttcctggattaaaaacggcgaagaaatattaggagctgacacgcagtatggaagcgaaggttcaatgaatagacctacagtttctgtacta |
| | agaaatgtcgaagttctcgataaaaacattggaatacttaaaacatctttagaaaccgcaaatagtgatattaaaacaattcagggcatcttagatgtatctggtgatattgaagctttggcccaa |
| | ataggtatcaataaaaaggatatttctgacctcaaaacgctaaccagtgaacatacagaaatattaaatggacctaatagtacagttgacaacattcttgctgatattggtccatttaactctgag |
| | gccaactctgtatacagaacaatcagaaatgatttactgtggataaagcgtgaacttggacaatacgcaggtcaagatattaatggtcttcctgttgtaggaaatcctagtagtggaatgaagc |
| | atcgcattattaataatactgatgccattacttcacagggaatacgtttaagcgaattagaaacaaaatttattgaatctgatgtaggttctttgactattgaagttggtaatcttcgtgaagagcttg |
| | gaccgaaaccaccatcattttcacaaaacgtttatagtcgtttaaatgaaattgacactaaacagacaacatttgaatctgacattagtgctattaagacctcaataggatatccaggaaataatt |
| | cgattattactagtgttaatacaaacactgataatattgcatctattaatttagagctaaatcaaagtggaggtattaaacagcgtttaaccgttattgaaacttctattggttcagatgatattccttc |
| | gagtattaaaggccaaatcaaagataatacaactttaatcgaatctctaaatggaatcgtcggtgaaaacacttcatctggtttaagagcgaatgtttcatggttaaacaaaattgttggaactga |
| | ttctagcggtggacaaccttctccttctgggtctcttttaaaccgagtttctacaattgaaacttctgtttcaggattgaataacgatgttcaaaacctacaagtagagattggtaataatagcgcag |
| | gaattaaagggcaagttgtagcgttaaatactttagtaaatggaactaatccaaacggttcaacagtcgaagaacgcggattaaccaattcaataaaagctaacgaaaccaacattgcatcag |
| | ttacacaagaagtgaatacagctaaaggtaatatatcttctttacaaagcggtgttcaagctctccaagaagccggttatattcctgaagcgccaagagatgggcaagcttacgttcgtaaaga |
| | cggcgaatgggtattgctttctacctttttatcaccagcataacatggggccgcaaggccccaaaggattttaaatgtcaggatataattctcagaatccaaaggaactcaaagatgtcattcta |
| | agacgtttaggggctccaattattaatgttgagttaacacccgatcaaatttacgattgtatccagcgtgccctagaattatacggtgaataccattttgatggactcaataaagggtttcatgtgtt |
| | ttacgtaggggatgacgaagaaaagtacaagaccggagtcttcgatttaagaggttctaacgtatttgcagtaactcgcattttacgcacaaatattgggtcaataacatctatggatggaaac |
| | gctacatatccgtggtttactgactttcttttgggaatggctggtattaatggcggaatgggaacgtcttgtaatagattttatggaccaaatgcctttggtgccgatttggggtattttactcaactt |
| | accagttatatgggaatgatgcaggatatgctctctcctattccagacttttggtttaattcagcaaatgaacagctcaaagtcatgggaaacttccaaaaatatgatttaattatcgtagaaagct |
| | ggactaaatcatacattgatacaaacaaaatggttggaaatacagtaggatatggaacagtcgttccacaagataactggtcattatctgaacgatataataaCccagacaacaatttagtag |
| | gtcgtgttgttggtcaagacccaaatgttaagcaaggtgcttacaataatcgttgggtgaaagactatgcaacagctttagctaaagaattaaatggtcaaattttagcacgccaccagggaat |
| | gatgcttcctggcggtgttacaattgatggacaacgcttaatagaagaagctcgattagaaaaagaagcactgcgcgaagaattatacttacttgaccctccatttggaattttggtaggttaat |
| | atggctacttacgataaaaatctttttgctaaattggaaaaccgcacaggttattctcagaccaatgaaactgaaatactaaatccttatgtaaatttcaatcattataaaaacagccaaatattagc |
| | tgatgtattagtagctgaaagcattcaaatgcgaggtgtagaatgctattatgttccaagagagtatgtttcccctgatttgatattcggcgaagacttgaaaaataaatttactaaagcttggaaa |
| | tttgctgcatatttaaattcatttgaaggatatgaaggagctaaatcgttctttagtaattttggtatgcaagtacaggatgaagttactttgtccattaatccaaacttgtttaaacaccaagtaaatg |
| | gaaaagaaccgaaagaaggcgatttgatatattttcctatggataacagcttatttgaaattaactgggttgaaccatatgatccattttatcaattaggccaaaacgctattcgtaaaattacggc |
| | aggtaaattcatttattctggagaagaaattaatccagttctacagaaaaatgaaggaattaacattccagaatttagtgaattagaattaaatcctgttcgcaatcttaacggtattcatgacatta |
| | atattgatcagtatgctgaagtagatcaaattaattctgaagctaaagaatatgttgaaccctatgttgttgtcaataacagaggcaaatctttcgaatctagcccatttgataatgatttcatggatt |
| | aa (SEQ ID NO: 446) |
| 36 | atgtttggttatttttataattcgtcttttagacgatatgctaccttgatgggcgatttgttttcaaatatccaaatcaaacgtcagttagaatctggtgataagtttatacgtgttcctattacatatgcat |
| | caaaggaacactttatgatgaaattgaataaatggacatcaataaattcacaagaagatgtagctaaagttgaaaccattctacctcgtataaatttacatttagttgattttagctataatgctccat |
| | ttaaaacaaacattttaaatcagaatttactgcaaaaaggtgcaacttctgtagtatcgcagtataatccatctcctattaaaatgatttatgaattgagtatctttactcgctacgaagatgatatgtt |
| | tcaaatagttgaacagattcttccatattttcaacctcattttaatacaactatgtacgagcagtttggaaatgatattccatttaaaagggatatcaaaattgtactgatgtctgctgctatagacga |
| | agctatagatggggataatttatctcgtcgtagaattgaatggtcattaacatttgaagtaaatggatggatgtatcctccagtagatgatgcagaaggattaattcgtactacttatacagattttc |
| | acgccaatacaagagatttgcctgatggcgaaggtgtttttgaatctgtcgatagcgaagttgttcctcgagatattaacccagaagactgggatggaacagtaaaacaaactttcactagtaa |
| | tgtaaatagaccaacaccgccagaacctcctggcccaagaacatagaggttattatggaaggtcttgatataaacaaacttttagatatttctgacctccccggaattgacggggaggaaatc |
| | aaagtatatgaacctctgcaattagtagaagttaaaagcaatccacaaaaccgtactcctgacttagaagatgattatggagtagttcgtcgaaatatgcattttcaacaacaaatgctaatgga |
| | cgcggccaagatttttcttgagacggcaaagaatgctgattctcctcgtcacatggaagtatttgcaactcttatggggcaaatgactacgacgaacagagaaatactgaagcttcataaagat |
| | atgaaagatattacatctgagcaggttggcaccaaaggcgctgttcctacaggtcaaatgaatattcagaatgcgacagtattcatgggttcaccaacagaattaatggacgaaattggtgat |
| | gcttacgaggctcaagaagctcgtgagaaggtgataaatggaacaaccaattaatgcattaaatgatttccatccgttaaatgaagctggaaaaattttaataaaacacccaagcttagcgga |
| | aagaaaagatgaagatggaattcattggataaaatctcagtgggatggaaaatggtatcctgaaaaattcagtgattaccttcgtctacacaaaatagtaaaaattccaaacaactctgataag |
| | cctgaattatttcaaacttataaagataagaataataaaagatctcggtatatgggtcttcctaacttgaaacgagctaatattaaaacacaatggactcgtgaaatggttgaggaatggaaaaa |
| | atgccgagacgatattgtttattttgcagaaacatactgtgctattactcatattgactatggtgtcataaaggttcaattacgtgactatcagcgtgatatgctcaaaataatgtcatctaaacgtat |
| | gactgtttgtaatctatcgcgtcagctcggtaaaacaacggtagtagctattttccttgcacactttgtatgttttaacaaggataaagctgtaggtattcttgcgcacaaaggctcaatgtctgcg |
| | gaagttttagaccgtactaagcaagcaattgaactgcttcctgactttttacagccaggtatagttgaatggaataagggttcaattgaactagataatggttcttcaattggcgcttatgcttcctc |
| | tcctgacgcagttcgtggtaactcgttcgcaatgatttacattgacgaatgtgcgtttattccaaacttccatgattcctggcttgctattcaaccagtaatttcatctggtcgtcgttcgaaaattatt |
| | attactacgactcctaatggattaaatcatttttatgatatttggactgctgctgttgaaggtaaatctggatttgaaccatatactgctatttggaattcagttaaagaacgtctttataacgatgaag |
| | atatttttgacgatggatggcaatggagcatacaaaccattaatggttctactttagctcaatttcgtcaagaacacaccgcagcgtttgaagggacttctggtacattaatttcgggaatgaaatt |
| | agctattatggatttcattgaagtaactccagatgatcatggttttcatcgatttaaaagccctgaaccagatagaaaatatattgcaactctagactgctcagaaggtcgtgggcaagattacca |
| | cgctttgcatattattgatgttaccgatgatgtgtgggaacaggttggtgttttgcactcaaacactatttctcatttaattctacctgacatcgttatgcgttatttagtagaatacaatgaatgccca |
| | gtttatattgaattaaatagtactggtgtgtcagttgcaaaatcgctttatatggatttagaatacgaaggtgttatctgtgattcatatactgatttaggaatgaaacaaactaaacgcacgaaagc |
| | agtaggatgttccacgctaaaagaccttattgaaaaagataagcttattattcatcaccgagcgactattcaagaatttagaacgtttagtgaaaaaggcgtgtcttgggcggctgaagaaggt |
| | tatcacgacgatttagtaatgtctttagtaatttttggatggttatcaacacaatcaaaatttattgattatgcggataaagatgacacgcgattagcatctgaagtattttcaaaagagcttcaagat |
| | atgagcgacgactacgcgccagttatatttgtggattcggttcattctgctgagtatgttccagtatctcatggtatgtcaatggtataaatatattaaagcatattaaagaggattaaaaatgacttt |
| | attatctccgggcattgagctcaaagaaactacggttcaaagcaccgtggttaataactctactggtacagcagctttggccggtaaattccagtggggtcctgcttttcagattaaacaggtta |
| | caaatgaagtagatttagttaatacttttggtcaaccaaccgctgaaactgctgactattttatgtctgcgatgaatttcttgcagtacggaaatgacttacgagtagttcgtgctgttgatagagat |
| | accgctaaaaactcatcaccaatcgctggtaatattgaatacacaatttctaccccaggtagtaactatgcggttggagataaaatcacagtcaaatatgtttcagatgatattgaaactgaagg |
| | taaaattactgaagtagacgcagatggaaaaattaagaaaattaatattcctactgcaaaaattatcgctaaagcgaaagaagtcggtgaatatccaacactaggttctaactggactgcgga |
| | aatttcttcatcttcctctggtttagctgcagtaataactcttggaaaaattattactgattctggtattttattagctgaaattgaaaatgctgaagctgctatgacagcggttgactttcaagcaaatc |
| | ttaaaaaatatggaattccaggagtagtagcgctttatccaggcgaattaggcgataaaattgaaattgaaatcgtatctaaagctgactatgcaaaaggagcttctgcattactcccaatttatc |
| | caggtggtggtactcgtgcatctactgccaaagcagtgtttggatatggaccgcaaactgattcacaatacgctattatagttcgtcgcaatgatgctattgttcaaagcgttgttctttcaactaa |
| | gcgtggtgaaaaagatatttacgatagtaacatctatatcgatgactttttcgcaaaaggcggctcagaatatatttttgcaactgcacaaaactggccagaaggcttctctggaattttaactct |
| | gtctggtggattatcatcaaatgctgaagtaacagcaggagatttgatggaagcttgggacttctttgctgaccgtgaatccgttgatgttcaactgtttattgcgggttcttgtgccggtgaatct |
| | ttagaaacagcatctactgtccaaaaacacgtcgtttcaattggggatgctcgccaagattgcttagtattgtgctctcctccgcgtgaaactgtagttggaattcctgtaactcgtgcagtagat |
| | aatttagttaactggagaactgcggcaggttcatacactgataataactttaatatcagttcaacctacgcagcaattgatggtaactataagtatcagtatgacaaatataatgatgtgaatcgtt |
| | gggttccattagcagctgatattgctggtttatgcgcaagaactgataacgtatctcagacttggatgtctccagctggttataatcgtggccagattcttaacgttattaaacttgctattgaaact |
| | cgccaggctcagcgcgaccgtttataccaagaagctatcaacccagtaactggtacaggtggcgatggttacgtattgtatggtgataaaacagctacttctgttccttctccatttgatcgtatt |
| | aacgttcgtcgtctgtttaatatgttgaaaacgaatatcggacgtagttcaaaatatcgtttgttcgaattaaacaacgcgtttactcgttcatcattccgcacagaaactgcccagtacttgcagg |
| | gaattaaagctctcggtggaatttatgaatatcgtgtagtttgcgatacaacaaataacactccgtcagtaattgatagaaatgagtttgttgcaacattctacatccaacctgcgcgcagtataa |
| | attatattactttgaatttcgtcgcaacggctactggtgcagatttcgatgagttaactggtcttgcaggttaa (SEQ ID NO: 447) |
| 37 | atgtttgtagatgatgtaacacgcgcgtttgaatcaggtgattttgcgcgacctaacttattccaagtagaaatttcttatcttggacaaaattttacgtttcaatgtaaagccactgctttaccagct |
| | ggtattgtagaaaaaattccagtcggatttatgaaccgtaaaattaacgtagcaggcgatcgtacattcgatgactggactgttacagtaatgaacgatgaagctcatgatgctcgccagaagt |
| | tcgttgattggcaaagcattgctgcggggcaaggaaacgaaattactggtggaaaacctgcagagtataaaaagagcgctatcgttcgtcaatatgctcgtgacgctaaaacagtaacaaa |
| | agaaattgaaattaaaggtctgtggcctactaacgtgggtgaacttcaattagattgggattcaaacaatgaaatccaaacatttgaagtaactcttgctctcgattattgggaataa (SEQ |
| | ID NO: 448) |
| 38 | atggctaaaatcaacgaacttctgcgcgaatcaaccacaacgaatagcaactcaatcggtcgcccaaatctcgttgctttgactcgcgctaccactaaattaatatattctgacattgtagcaac |
| | gcaaagaactaatcaacctgttgctgctttttatggtatcaaataccttaacccagacaacgaatttacatttaaaactggtgctacttatgctggcgaagctggatatgtagaccgagaacaaat |
| | cacagaattaacagaagagtctaaattaactctcaataaaggcgatttattcaaatataataatatcgtttataaagtattagaagatacaccatttgctgatattgaagaaagcgacttagagctg |
| | gctcttcagattgcaattgttcttttaaaggttcgtctattttctgacgcagcgtcaacaagcaaatttgaaagctctgatagtgaaattgcggatgctagattccagattaataaatggcaaaccg |
| | cggttaaatctcgtaaacttaaaactggcatcacagttgaattagcgcaagatttagaagcaaatggattcgatgctcctaatttcttggaagatttgcttgcaactgaaatggcagatgaaatca |
| | ataaagatattctgcaatctttgattacagtgtcaaaacgctataaagttacaggaattactgatagtggattcatcgatttgagttatgcgtctgcacctgaagctggtcgttcattataccgaatg |
| | gtatgtgaaattgtttcgcatatccaaaaagaatcaacttatacagcaacgttctgtgttgcttctgctcgtgccgctgcgattcttgctgcatcaggttggttaaaacataaaccagaagatgac |
| | aaatatctttcacaaaatgcctacgggttattagctaatggtttaccgctttattgcgatactaacagcccattagattatgtaatcgttggtgtagtagaaaatatcggtgaaaaagaaattgttgg |
| | atcaattttctatgctccgtatacagaaggtctcgacttagatgaccctgaacatgtaggcgcatttaaagttgttgttgatccagaaagcttacaaccgtctatcagtttattagttagatatgcttt |
| | atcagcaaatccttataccgtagcaaaagatgaaaaagaagcaagagtaattgatggtggagacatggataaaatggcgggtcgttcagatttgtctgttttattaggtgttaaattaccaaaaa |
| | ttattattgatgaataa (SEQ ID NO: 449) |
| 39 | atgagaactgaggttgtggtgtttactcttcatgagtctggaaagtcattcattgaaattgctcgtgaattaaacttacatgcaaaagaagtggctgtattatgggctcgagctatgactgctaag |
| | aataaatttgaaactcgagaaaaagttgtctatagaaaaagacatatcaataaaaaggtgaaaaatggaacagtatgaactttatgaaaatgaatcttttgctaatcaattacgcgaaaaagcat |
| | taaaaagtaaacagtttaagctagagtgttttattaaagatttttcggaacttgctaataaagcagctgaacaaggtaaaacatattttagttattatactgctcgcgataaattgattactgaagaa |
| | attggtgattggctgagaaaagaaggatttaattttaaagtcaatagtgatcagcgtgatggtgattggttagaaattacattttgaggattaattatgtttaaaaagtagcagtcttgaaaatcatta |
| | caactctaaatttattgaaaaactttacagcttgggattgactggcggcgaatgggtagctcgtgaaaagattcacggcacaaatttctcattgattattgagcgtgataaagtaacttgtgctaa |
| | acgtactggaccgattcttcctgctgaagatttctttgggtatgaaattattctaaagaattacgctgattccattaaagcagtacaagatattatggaaacctcagcggttgtatcttatcaagtctt |
| | tggcgaattcgctggacctggcattcagaagaatgttgattatggcgataaagatttttatgtatttgacattattgtcactacagaaagtggtgatgtgacttatgttgatgattatatgatggaatc |
| | attctgtaatacatttaaatttaaaattgctccacttttaggtcgcggtaaatttgaagagcttattaaattgccaaatgatttagattctgtcgtccaagattataattttacagtagaccatgctggatt |
| | agttgatgcaaataaatgcgtttggaatgccgaagcaaaaggcgaagtatttactgctgaaggatatgtattgaaaccttgttatccttcttggcttcataatggaaatcgtgtagcaattaaatg |
| | caagaattccaaatttagtgaaaagaaaaagtctgataagcctattaaagctaaagttgaactatcagaagctgataacaaattggtgggaattttagcttgttacgttacactgaaccgtgtaaa |
| | taacgttatttctaaaattggcgaaattggtccaaaggattttggaaaggtgatggggctaactgttcaagatattttggaagaaacttctcgtgaaggtattactctaactcaagcagataatcct |
| | tctttgattaaaaaggaattagttaaaatggtaagatgtacttcgtccagcttggattgagttggtgagctaa (SEQ ID NO: 450) |
| 40 | atgatagataaagattatattgcagagctgaaggctcttgatgataacaaagaagctaaagctaaattagctgaatatgctgaacagtttggtataaaggtcaaaaagaataaatcttttgataat |
| | atcgttgttgatattgaagaagccctccagaagctcgctagtgaacctatgccagagactgatgggttatctattaaagacttaattgatgctgctgatgccgcagagggattaaaatatgacg |
| | atgaagaagtcaatccagaagcagcacttctgattgattctccggttaaatctgacattaaaattgaagtagtagaaacggataaaattcctgaaaataccgatgttttgattgaagatactccttt |
| | tgttgaagaaaagtttgaacaagctgtagctgagattattgaatctgaaaagccgtctgtatttactcttccggaaaactttagtccgaatcttcagctgattggaaaaaatccaggattctgcact |
| | gttccttggtggatttatcaatggattgctgaaactccggattggaaatctcacccaactagttttgaacatgcgtcagcacaccaaactttatttagcttaatttattacattaaccgcgacggatc |
| | agttttaattcgtgaaacacgcaattcttctttcgtaacattaaaataaggataacttatgacttttacagttgatataactcctaaaacaccgacaggggttattgatgaaaccaagcagtttactg |
| | ctgcacccagtggtcaaactgaaggtggaactattacctatgcttggagcgtagataatgttccacaagatggagctgaagcaacttttagttatacctgccggtcaaaagactattaaagtag |
| | ttgcaacaaatacaattccagaagctgaagctgaaacagcagaagctactacaactatcacagttcaaaataagacacaaacgaccaccttagctgtaactcctaatagccctgacgctgga |
| | gtaatcggaaccccagttcaatttactgctgccttagcttctcaacctgatggagcatctgctacgtatcagtggtatgtagatgattcacaagttggtggagaaactaactctacatttagctata |
| | ctccaactacaagtggagttaaaaaatcaagtgtgtagctcaagtaaccgcgacagattatgatgcactaagcgttacttctaatgaagtgtcattaacggttaataagaagacaatgaatcca |
| | caggttacattgactcctccttctattaacgttcaacaagatgcttcggctacatttactgctaatgttactgatgctccagaagaagcgcaaattacttattcatggaagaaagattcttctcctgta |
| | gaagggtcaactaatgtatataccgttgatacttcatctgttggaagtcaaactattgaagtgactgccgtcgttactgctactgattatgatagcaaaacagttaaaacaacaggtcaagttcag |
| | gtaactgataaagttgctccagaaccagaaggtgaattaccttatgttcatcctcttccacatcgtacttcagcttacatctggtgcggttggtgggttatggatgaaatccaaaaaatgactgaa |
| | gaaggtaaagattggaaaactgaagatccagagtaaatactacctgcatcgttacactcttcagaagatgatgaaagactatccagaagttgatgtccaagaatcgcgtaatggatacatcat |
| | tcataaaactgctttagaaactggtatcatctatacctatccataa (SEQ ID NO: 451) |
| 41 | atgagattagaagatcttcaagaagaattgaagaaagatgtgtttatagattcaactaaattacagtatgaagcagctaataatgtgatgttatacagtaaatggcttaataagcattcaagtatta |
| | aaaaggaaatgcttagaattgacgcacagaaaaaagttgctcttaaagctaaattagactactactcgggacgaggagatggtgatgaatttagtatggatcgttacgagaaatcagaaatga |
| | agacagttctatcagcggataaggatgttttaaaggttgatacctcgttacagtattgggggattttattagatttctgtagcggagctcttgatgctatcaaatcacgcggatttgctattaagcat |
| | attcaagacatgcgggcatttgaggctggaaaataatgagatatagcattgatgatgcttttaattatgaagaagaatttgaaactgagattcaattcttaatgaaaaagcataatcttaagcgtc |
| | aggatattcgtatcctggccgatcacccgtgtggtgaagatgtcctttatattaaaggaaaatttgccggatatcttgatgaatatttttattctaaagatatgggcattgatatgcatatgagagttg |
| | tataaatagatataattcagaggagacaatcatgtcagataagatttgtgttgtctgtaaaactccaatcgattctgcattggttgttgaaacagacaaaggtcctgtacatcctgggccttgctat |
| | aattacattaaagaactaccagtttcagaaagttcggaagaacaattaaatgaaacacaacttttgctatag (SEQ ID NO: 452) |
| 42 | atgtatgaatacaaatttgatgtgagagttggttctaaaataatcaattgtcgcgcattcacgcttaaagaatatctagaacttattactgccaaaaataatggttccgtagaagtaattgttaaaaa |
| | gctaatcaaagactgcacaaatgcaaaagatttaaaccgccaagaatcagaactattgctgattcatttatgggcgcattctcttggagaagttaatcacgaaaactcctggaagtgcacctgt |
| | ggaactgaaataccaacccatataaatctattacatacacaaatagatgcaccagaagacctctggtatacactgggtgacattaaaattaaattccgataccctaaaatttttgatgataaaaat |
| | atagcccacatgatagtatcatgcatagaaacgattcatgctaacggtgaaagcattccagttgaagacttaaatgaaaaagaactagaagatttatattctatcatcacagagtcagatattgt |
| | agctataaaagatatgcttttaaagcctaccgtttatttggctgttccaattaaatgtccagagtgtggaaaaacccatgctcatgtaataagaggcctcaaagagttctttgagttactataatgg |
| | caaatattaataagctttattctgacattgacccggaaatgaaaatggattggaacaaagatgtttccagatcacttggattaaggtcaattaaaaacagtcttttgggaattattacaacaagaaa |
| | aggttcaagaccgtttgaccctgaatttggatgtgatttatcagatcagctttttgaaaatatgactcctcttactgctgacacggttgagcgcaatatcgaaagcgcagtaagaaactatgagc |
| | cacgtattgataaattatcagttaatgtgataccagtttatgatgattatactctgatagtagaaatacgcttttcggtcatcgataaccctgatgatattgagcagataaaactgcaactggcttcg |
| | agtaatagggtataa (SEQ ID NO: 453) |
| 43 | atggcaaacattattcgttgtaaattaccagatggtgttcatcgttttaaaccatttacggtagaagattatcgagattttttgttagttcgaaacgatatagaacatcggtcaccacaagaacaaaa |
| | agaaataattactgatttaattgatgattattttggagactatccgaagacttggcaaccatttatatttttgcaggtatttgtagggtcaataggtaaaactaaagtaccggtcacatttgtatgtcca |
| | aaatgtaaaaaagaaaagacagttccatttgaaatatatcaaaaagaattaaaggaacctgtttttgatgtagctaatgttaaaattaaattaaagtttccttctgagttttatgaaaataaagcaaa |
| | gatgattactgaaaatattcattctgttcaagtagatgaaatatggtatgattggaaggaaattagtgaatcaagccaaatagaacttgttgatgccatcgagatagaaacattagaaaaaattct |
| | cgatgcaatgaatcctattaatttaactctacatatgtcatgctgtaataagtacattaaaaaatacactgatatagtagacgtgtttaagctgttagttaacccagatgagatatttactttttatcaa |
| | attaatcacacactcgtaaaaagtaattatagcttaaattcaataatgaaaatgattcctgccgagcgcggattcgtattaaaactgattgagaaggataaacaataatgagtatgttgcaacgc |
| | cccggatatccaaatctcagcgttaaattatttgatagctacgacgcttggagtaataatagatttgttgaattagctgctactattaccacattaactatgcgggattctctttatggacgaaatga |
| | aggaatgctgcagttttatgattctaaaaacatccatacaaaaatggatggaaatgaaataattcagatttctgtagctaatgcaaatgatattaataatgttaaaacacgaatttatggatgtaag |
| | catttttccgtgtcagtagattcaaaaggtgataacatcattgctattgaattgggaactattcattctatagaaaatcttaaatttggtagacaatttttccctgatgcaggtgaatctataaaagaaa |
| | tgcttggtgtcatttatcaggatcgcacattattaactccagcaataaatgctataaatgcttatgttcctgatattccatggactagcacatttgaaaactatttgtcatatgtaagagaagttgctct |
| | agctgtaggaagcgacaaatttgtatttgtatggcaagacatcatgggagttaacatgatggactatgatatgatgataaatcaagaaccatatccaatgattgtcggtgagccatctttaatag |
| | gtcaattcatccaagaattaaaatatccattagcatatgatttcgtttggttgactaaatcgaatccttacaaacgtgatccaatgaaaaatgctactatctatgctcattcatttttagattcttcactg |
| | ccaatgattactacaggaaagggtgaaaactctattgtagtgtcaagatcaggtgcttattctgaaatgacttataggaatggatatgaagaagctattcgtcttcaaactatggcacaatatgac |
| | ggttatgctaaatgttctactgtcggtaattttaacttgactcctggtgttaaaattatttttaatgatagtaaaaaccaatttaaaacagaattttacgttgatgaagttatccatgaattatccaataat |
| | aattcagtaactcatctatatatgttcactaatgcaacgaaactggaaacaatagacccagttaaggttaaaaatgaatttaaatctgatactaccactgaagaaagtagttcttccaataagcaa |
| | taaagaagtttctattcctaaaatgggtcttaaacattataacattttaaaggatgttaaaggtcctgatgaaaatttaaaacttcttattgattctatttgtccgaatttatcaccggcagaagttgattt |
| | cgtttctattcatttattggaatttaatggaaagattaaatctcgtaaagaaatagatggctatacttatgacattaatgatgtttatgtatgccaaagattagaatttcaataccaaggaaatacatttt |
| | attttagacctcctggaaaatttgaacaatttttaacggtgagcgatatgttatctaaatgcttgcttaaggtcaacgatgaagttaaagaaattaattttcttgagatgccagcattcgttttaaaatg |
| | ggcaaatgatatttttacaactttagcaattcctggccctaatggtccaataaccggaattggcaatattattggattatttgaatgaaaaagccacaagaaatgcaaacgatgcgtagaaaagtt |
| | atttcagataataaaccaacacaggaagcggctaaatccgcttctaacactttatctggacttaatgatatatctacgaaattggatgatgctcaagctgcttctgaattaatagctcaaactgtcg |
| | aagaaaaatcgaatgaaatagttggagcaattggtaacgtagaaaacgcagtgagtgatactactgccggttctgagttaattgctgaaactgtcgaaattggcaacaatattaataaagaaat |
| | cggtgaatcactcggaagcaaattagataaattaacaagtttactagagcaaaaaattcagacagctggaattcaacagactggaactagtttagccacagttgaaagcgctattcctgttaaa |
| | gtcgttgaggatgatactgctgaatctgtgggtcctttattaccggctcccgaagcagttaataatgatcctgacgctgattttttccctacccctcagccagttgaacccaaacaagaatcgcc |
| | agaagaaaaacagaaaaaagaagcatttaacttaaaattatctcaagctttagataaattaacaaagactgttgattttggatttaagaaatccatttcaattagtgataaaatatcaagcatgttat |
| | ttaagtacaccatcagtgctgctattgaagctgctaaaatgactgcaatgatattggctgttgttgttggaatagacctgttgatggttcactttaaatattggtcagataaattttcaaaagcctgg |
| | gatttatttaatactgactttactaaattctctagcgaaaccggaacttggggtcctttattacagagcatctttgattctattgataaaattaaacaactttgggaagcgggagattggggtggatt |
| | gacagtagctattgttgaagggcttggaaaggttctttataatttaggagaacttattcagcttggaatggctaaattatctgcggcaattcttcgagtcattcctggcatgaaggatactgctgat |
| | gaagtagaaggaagagcactagaaaatttccaaaattctactggagcatctctcaataaagaagaccaagaaaaagtagcaaattatcaagataaacgaatgaatggagaccttggcccaa |
| | tagcagaaggactagacaaaatctctaactggaaaactcgtgcatctaactggattcgtggtgtagataataaagaagcactgactactgacgaagaacgtgcagcagaagaagaaaaatt |
| | aaagcagctttcacctgaagaaagaaaaaatgctttaatgaaggccaatgaagctcgtgccgcgatgattcgttttgaaaaatatgctgattcagctgatatgagtaaagactcaacggttaaa |
| | tcagttgaagctgcctatgaagaccttaaacagcggatggatgacccggatttaaataattcgccggcagttaaaaaagaacttgcttctagatttgctaaaattgatgctacttatcaagagct |
| | caagaaaaatcagcctaatgccaaacctgaaacttctgctaaatcaccagaagcgaaacaggtccaggttattgaaaagaacaaagcacaacaagctcctgttcaacaagcatctccttca |
| | atcaataatactaataatgttattaagaaaaatactgtcgttcataatatgacacctgttacgagcacaactgctcctggtgtatttggcgcgactggagttaattaaggaataatatggcaattgtt |
| | aaagaaataactgctgatttaattaaaaagtccggtgagacaatttcagccggacagagcactaaatcagaagtaggaattaaaacatacacagcccagtttccaactgggcgtgctagtgg |
| | taatgacactacaggggacttccaggtaacagatctatataagaatggattattatttactgcatacaatatgtcatctagggattctggaagtcttagatcgatgagatctaactactcttcttcat |
| | cttcgagtattttacgtacagccagaaacactattagtagtacagtatcaaaactatcaaatggattaatatcaaataataattcaggaacaataagtaaagctcctgtcgcaaacattcttttacc |
| | gagatctaaatctgatgttgatacatcatcacatagatttaatgatgttcaagaaagccttatcagtagaggcggaggtactgctactggagtgctaagtaatattgcttcaaccgcagtatttgg |
| | ggcgttggaaagtataacacaaggtataatggctgataataatgaacagatttatacgacagccagaagtatgtatggtggtgctgaaaatagaactaaagtgtttacatgggatttaactcca |
| | cgttcaacagaagatttaatggctattattaatatctatcaatattttaactatttttcttatggtgaaacgggtaaatctcaatatgctgctgaaataaaggggtatttagatgattggtatcgttctac |
| | gttaattgaacctttatctccggaagacgcagctaaaaataaaacactatttgagaaaatgacatcgagtttaactaacgttctagtagtttcaaacccgacggtttggatggtgaaaaactttgg |
| | tgcaacatctaagtttgatggaaaaacggaaatatttggtccatgccaaatacagagcatcagatttgataaaacacctaacggtaactttaacggattagctattgctccaaatctccctagtac |
| | atttactctcgagattactatgagagaaattatcacgttaaaccgtgcttctttatatgcggggactttttaatgtattctttagaggaatttaataatcaagcaataaacgcagatttccaacgtaata |
| | atatgtttagctgcgtttttgcaacaactccatcaactaaaagctcttcgttgataagttcaattagcaacttttcttataataacttgggcctaaattcagattggttaggattaactcaaggtgatatt |
| | aatcagggaattacaacgctaattacagctggcacacaaaaactaataagaaaatcgggggttagtaaatatcttattggtgccatgagtcaacgtacagttcaaagtttattaggctcatttac |
| | agttggtacatatttaattgacttctttaacatggcatataactcatctggattgatgatatactctgtaaaaatgccagagaatagattatcctatgaaactgattggaactacaactctcctaatatt |
| | cgtataactgggagagaattagaccctttggttatttcatttagaatggattcagaatcgtgtaattaccgtgcaatgcaagactgggttaatgctgttcaagacccagtaactggattacgtgct |
| | ctgccacaagatgtcgaggcagatatccaggttaatcttcattctcgtaatggattgcctcatactgcggtgatgttcaccggatgtattccagtgtcagtgagcgctcctgagttatcatatgat |
| | ggagataaccaaataactacatttgatgttacttttgcgtatagagtcatgcaggctggagcagttgataggcaagctgcgcttgaatggcttgaatctgctgctataaatggtattcaaagctct |
| | tctggaaataatggaggtgttactgaactatctagttcgctttcacgacttagtagattaggaggaactgcaggaagcatttcaaacattaatactatgacagggattgtcaattcgcagagtaa |
| | aatattaggagcaatataa (SEQ ID NO: 454) |
| 44 | atgaaatcttctttgcgctttttaggtcaagaacttgtagttgaaggcgttattcctgctgataatgcttttaacgaagcggtttacgatgaatttattaaaatttttggaacagataaaaagttcggaa |
| | tttttccttctgaaaatttttcaaagccagaacagactgaaagcattttccagggtgtagtaacaggtaaatttgagtcagaagctccggtaaaaattgaagtttatattgaagacagtttagttgct |
| | tcagtttctgctttcatttcattccgtaaataa (SEQ ID NO: 455) |
| 45 | atggaactcattacagaattatttgacgaagatactactcttccgattacaaacttaaatccaaagaagaaaataccacaaattttttcagttcatgttgatgatgcaattgaacaaccaggctttc |
| | gtttatgtacctatacatctggaggtgatactaatcgcgatttaaaaatgggcgataaaatgatgcatattgttccttttacattaactgctaaaggttcaattgctaaattaaaaggtcttggtccaa |
| | gcccaattaattatatcaattcagtttttactgttgcaatgcaaacaatgcgtcagtataaaattgatgcttgtatgcttcgtattcttaagtctaaaactgctggtcaagctcgacaaattcaagttatt |
| | gctgatagacttatccgtagtcgttcaggtggcagatacgtccttcttaaggaactctgggattatgataaaaagtatgcatatattcttatacatcgcaaaaatgtatcactagaagacattccag |
| | gagttccggaaattagtaccgagctctttactaaagttgaatcgaaggtcggtgatgtttatatcaataaagatactggagctcaagtaactaaaaacgaggcaattgcagcatctattgcaca |
| | agaaaatgataaacgtactgaccaagctgtaatcgttaaagttaaaatttcccgtagagcaattgcgcaaagtcaatcattggaatcttctagatttgaaagtgaattattccagaagtatgaatc |
| | taccgcagctaatttcaataagcctgctaccgctcctttaattcccgaagcagaagaaatgaaaattggaattaattcattagcttctaaaacaaaggcagcaaaaattattgccgaaggaact |
| | gcgaatgaacttcactatgactataaattcttttcaaaaagtgaggttgatgaagtttctgaaaaaattaaagatgtaatttttaacgcgattaaaaatgaaccaactacttcaataaaatgtttaga |
| | gaaatacgcggcagctgtcaatcaattctttgaagaatataaagataattggcttgataaacataataaaactcgtaaagggcagccagatgaagtctggggagaaataactaaaaatgcctg |
| | gaatgcagcaaaaactaaattcctcaaacgaatgatttatagtttttctggaattggtgctggtccaatgattgatattactattgcttgtgatggttctaaatatacaccatcacaaaagcgcggta |
| | ttagagagtattgtggttcaggatatacagacattaataatcttcttttaggtcgttacaatccagaacgatatgatgtaatgagtgaaaaagaaattgaatctgctataaataatttagattcagctt |
| | ttgaaaatggtgaccgcataccggaaggcattacagtttatcgtgctcaaagtatgactgctcctatatacgaagcgctagttaaaaataaagtgttctatttcagaaattttgtatctacttctttaa |
| | ctcctatcatttttggacgttttggaattacacatgctggtattggtcttttagaaccagaagctcgcaatgaattaacagttgataaaaatgaagaaggaataactattaatccaaacgaaataag |
| | agcgtataaagaaaatcctgaatacgttaaagttcaaataggatgggcaattgatggagctcataaagttaatgttgtatatccaggaagtctcggaatagcaacagaagctgaagttattcta |
| | ccgcgcggattgatggtcaaagttaataaaataactgatgcttctaataatgacggaaccacgtctaataatacaaaactcattcaagctgaagttatgaccacagaagaactcaccgaatcg |
| | gtaatctatgacggagaccgtttaatggaaaccggcgaagtagttgcaatgacaggtgatattgaaatagaagacagagttgactttgcatcatttgtttcatcaaatgttaaacagaaagtag |
| | aatcatctctcggaattattgcgtcttgcatagatattacaaacatgccttacaagttcgttcaaggataaatcatggaacttattacagaattatttgacggcgcttcggcgccggttgttaactta |
| | aatcctaagcataaaataccacaaatttttgctattcaagccggcgaagaaagcgtgcttcctggatttagattttgtacatacacctctggtggtgatacaaataaaaacgttaagccaggcga |
| | taaaatgatgcatatcgtaatgataggtgtcaacgagaaattatcgctggttaagcttagaaacttgggtggaaatccaattggcgtcattaatgctgtttttgatactgctcttcaaacaatgaaa |
| | cagtataaaatcgacgcatgcttattccgcgtactaaaaagtaaaacaaatggcgcagctcgtcaaatgcaagttattgctgaccgtttagtacgtactaaaggagcaggtcgatatgttctttt |
| | aaaggaaatctgggactatgataaaaagtatgcatatattatggtttaccgtaaaaatgccaatttagaagacattccaggtgtacctcctatttcaactgagttattcgcaaaagttgaatcgaa |
| | ggtcggtgatgtttatgtagatgttaaaacaggtgatgctgttcctaaagctgtcgctgttgctgcttctattgctttagaaaatgataaacgtactgaccaagcggttattcagaaaactaaaatta |
| | gtcgtcgattagcagcacaagctcaatattctactgtcgatgcttcacttcagggtgatagcttcgctgccaagaaatatcaagagtttgaatctaaagttccggtatataaagcagaaggacc |
| | aatgaactctggcgttattcagattggttcaaacttcagcaaaggagctatcggtggtatgagaagtgcttctcgttttaaatctagcgattatgaactagaaaacttccgaaatcatattgcatta |
| | gcccatgcacgtttacgtgatccatctatcaagttacagagcgatataacatatcaaggttctcaagaatatttaaagaataaagaattctttgattataaaactgataaaattttaagtgatcttgct |
| | gatattaatatttctaatagctttgatgttattaagaaaattatcaatgatttggttaaaggttctaaagctacgccagatgaaaagacagttattattcaatttgtcatgaatggcatttataaattgatt |
| | aatgaatctgctgcccaggcatatgaatatgcaagcactgaagtaactccaaaaggactgactcaggctgagtctgatgtaattgaagattattgtgcagattcatatgttgaaatgaactcgtt |
| | ccttttgggtaaaccagattctacccgtgaagaatatatggaacgtgctattaagcacatcgagacgttggattctgcattcgctaaaggttcagttcttcctccaggaactacgctttatcgcgg |
| | acaagaagttacctttaaaactttgcgtcacaacattgaaaacaaaatgttctatttcaagaacttcgtatcgacatcacttaaaccaaatatctttggcgagcatggtaaaaactatatggctcta |
| | gatgattccggtgcagtattttctggagaaggagaaggttccgttgatgcagaagatttgatgcatatgggtagtcattctacatatgctaatgaagatgctgaaactagcgtgggtatggtaat |
| | taaaggagctgagcgaatcaaagttatcgttccaggtcatttatcaggatttccatcagaagctgaagttattctaccgcgtggaattttactgaagattaataaagtaagtacgtactttatgaaa |
| | gaaactgcttataacaagtatctaatcgaaggtacaatcgttcctccttctgaacaattagaagaatcagtatatgatggagaccatttaatggaaactggtgaagttcgtccaatggctggattt |
| | aatcaattccttgtagaagaatcaaaagaagaggaaaacgaagtttctcaaatattagcttctttggttaacatcaacggaatgtctaaaaagttcaaaatgtag (SEQ ID NO: 456) |
| 46 | atgaactacatcaactttgaacgtaaatatgtttctaatggtattgcaggttctattgatactatctgcctttggaaacatcaaaatggatcagtatgcgaaattgaacagtatatgactcctaactat |
| | gtttatatgcgatttgaaaatggcatcacggtttcaatcacaatggaaggttccaactttaaaatcgctctggatgatgattttcgtcaacgcgatttagggactcatccttgctggaatggtgcta |
| | atcgcaagcttttggttaaaacttggattcgtcatattctgagtaacagagctaaacctgagcatcttgaagcaatctttgatgtagttcttaacgaatttgatatttaa (SEQ ID NO: 457) |
| 47 | atggcaaaacaagctaaagcaaagaaagcagttgaaaagaaagttggtgattctaaacgcgctggctacaagcgtgggtcgaactctcgtatcaatcaaactgttgagaagatcatgcgcc |
| | gagcacgtgcggttcttcgagatgatgcttctcgttttggtaagcagaaagcataa (SEQ ID NO: 458) |
| 48 | atgattaaacaattacaacacgctcttgaactgcaacgaaacgcatggaataatggtcacgaaaactatggcgcatctattgatgttgaagccgaagctcttgaaatcctgcgttatttcaaaca |
| | tctgaatcctgctcaaactgcattagctgccgagcttcaggaaaaagatgaacttaaatatgctaagcctctggcttctgccgcgcgaaaagcagttcgtcactttgtggtaacattgaagtaa |
| | (SEQ ID NO: 459) |
| 49 | atgtctgaagtacaacagctaccaattcgtgctgtcggtgaatatgttattttagtttctgaacctgcacaagccggcgatgaagaagttacagaatcaggacttattatcggtaaacgtgttcaa |
| | ggtgaagttcctgaactgtgtgtaattcactctgtcggtcctgatgttcctgaaggtttttgtgaagttggtgatttgacttctcttccagttggtcaaattcgaaacgttccgcatccttttgtagctct |
| | gggtcttaagcagccaaaagaaattaaacaaaaattcgttacctgtcattataaagctattccgtgtctttataagtga (SEQ ID NO: 460) |
| 50 | atgctgctaagtgaaaaaccgattactgttaaagaattccaagaaaaagttaagctatttgcgcaggaattggtaaataaggtttctgaacgatttcctgaaacatcggttcgtgttattaccgaa |
| | actcctcgttcagtattagtaattgtgaatccaggtgatggcgatcaaatatcgcatcttaaactggattttgatggattagttgaagcacaaagggtgtatggcgtactatgatgaatttaactga |
| | tataattgataattgtcttgaaaatgatactggcgatcatagagcgcttgactctgaaacagcaaagttcattagaataactttaatgaatgatactctggtgaatagtattcatccttctgtgtatga |
| | tgctattattgtgacgaagtatccagttgagcttcataaaaagatgactggcgcagtttttattgataagaaaaaccgctttaaagatgggcagaatataattagttctgttattaaaagtataacta |
| | aacttcgtcacgaaatttatcgtgttgaaactgctaaatctgcttatctggtgattatgaaatgaaagcgagtacagtacttcaaattgcatatttagtatcgcaggaatcaaaatgttgctcctgga |
| | aggtaggagcagtaattgaaaagaatggacgtattatttctactgggtataatggttcacccgcagggggtgtgaactgttgtgattatgctgctgagcaaggttggttgctgaataagcctaa |
| | acatactatcattcaaggccataagcctgaatgcgtatcatttggttcaactgatcgttttgtcttggcgaaagaacatcgtagtgctcactctgaatggtcgtctaaaaatgaaattcatgctgag |
| | ctaaatgcaattttgtttgctgcacgaaatggttcttctattgaaggtgctactatgtatgtaacactttctccttgtccagattgtgcaaaagcgatagctcaatctggtattaaaaagctggtttatt |
| | gcgaaacatatgataaaaataaacctggctgggatgatattctgcgaaatgcaggtattgaagtgtttaatgttcctaagaaaaacttgaataagttaaactgggaaaatatcaacgaattctgc |
| | ggtgaataatgaaatttcgtttggtaaagctcacagcaattagttcttattctaatgagaacatctcgtttgctgtagagtataagaaatattttttctctaaatggaaacagtattataagacaaattg |
| | ggtttgtattgataaaccatatagttggaaatctgatttagaaaaattccaaaaattactttccacccttaaagaacgtggaacaactcatattaaaactgtaataggtaaataaatgaaactgaca |
| | actgagcagaaagtagcaattcgtgaaattttgaaaactaaattgtccatgggtgtttcaaacgtagtttttgaaaagtctgatggtactattcgtactatgaaaggtactcgtgatgcagactttat |
| | gccaaccatgcaaaccggtaaattgactgaatctactcggaaagaatctacggatatgattccagtatttgatgttgaacttggcgcttggcgaggtttttctattgacaaattgatttctgttaatg |
| | gtatgaaagttgagcatttgcttcaatttattggtaaataa (SEQ ID NO: 461) |
| 51 | atgtttcctacttattctaaaatcgtagaagtagtgtttagccaaattatcgctaataatatgtttgaaaaacttgataacgcagccgagcttcgaatccatgctcaagtgactcatgtattgaacact |
| | ttgcttccagaccaggtggattctgttgccattacgctgtatccaggttccgcgcatatcattgttgtattcggtcttgatgctgagctagtcatcaaaggcgatattcgttttgaatcgcagacag |
| | cagaattcaaagcaatttaa (SEQ ID NO: 462) |
| 52 | atgaaacaataccaagatttaattaaagacatttttgaaaatggctatgaaaccgatgatcgaacaggcacaggaacaattgctttgttcggtactaaattacgctgggatttaagtaaaggtttt |
| | cctgcagtaacaactaaaaagctcgcctggaaagcttgcattgctgagctactttggtttttatcaggaagcacaaatgtcaatgatttacgattaattcagcatgattcattaattcaaggcaaa |
| | acagtctgggatgaaaattacgaaaatcaagcaaaagatttaggataccatagcggtgaacttggtccaatttatggaaaacagtggcgtgattttggcggtgtagaccaaattgtagaagtt |
| | attgatcgtattaaaaaactgccgaatgataggcgacaaattgtttctgcgtggaatccagctgaacttaaatatatggcattaccgccttgtcatatgttctatcagtttaatgtgcgtaatggcta |
| | tttggatttgcagtggtatcaacgatcagtagatgtttttcttggtcttccatttaatattgcatcatatgctgcgttagttcatattgtagctaagatgtgtaatcttattcctggagatttgatattttctg |
| | gcggtaatactcatatctatatgaatcacgtagaacaatgtaaagaaattttgcgtcgtgaacctaaagagctttgtgagctggtaataagtggtctaccttataaattccgatatctttctactaaa |
| | gaacaattaaaatatgttcttaaacttaggcctaaagatttcgttcttaacaactatgtatctcacccgccaattaaaggaaagatggcggtataattttaatttaattgcgaggatatatgattttac |
| | gatttaaagatacttctggtgtcgttctttttacacttcctaatccaagcgagttagaagttccaggaccaaatcagcctattatcatttatggcaaaaaatattatactcataaaatgactcgtgagt |
| | attttgataataaaatttctacagttaaaacttcttcagattgttactatgatattactgttttaacggaaaaacaatatgacgaattatcgccgcgcgggccgtctatgccaggtagtgaataaatat |
| | aaatccgactttgatgttaatattcaccgtggtacattttggggaaattacgtcggtaaagatgctggcagccgggaggctgccattgaattattcaaaaaagattttatacgtcgaattaaatcc |
| | ggagaaataactaaagaacatttagagcctttacgtggaatgaggctaggatgcacatgtaaaccaaagccgtgtcatggtgatataatagctcatatagttaaccgattgtttaaagacgattt |
| | tcaagttgaggacttatgcaattaattaatgttatcaaaagtagtggtgtttctcagagctttgacccgcaaaaaattattaaagttttatcttgggcagctgaaggaacatctgtagatccttatga |
| | attatatgaaaatattaaatcatatctccgtgatggaatgaccactgatgacattcagactattgtcattaaggctgctgcgaattctatttcggttgaagaacctgattatcaatatgtagctgcac |
| | gctgtttaatgtttgctcttcgtaaacatgtttatgggcagtatgaaccgcgttcatttattgaccatatttcttactgtgtaaatgaaggtaaatacgaccctgaattgttgtcaaaatattctgcaga |
| | agaaattacatttttagaatcaaaaattaagcacgagcgggatatggaatttacttattccggggcgatgcaattaaaagaaaaatatctcgttaaagataaaaccactggtcaaatttatgaaa |
| | ctccacagtttgcatttatgactattggaatggcattgcatcaagatgaacctgttgacagattaaaacatgttattcgtttttatgaagcagtatctactcgacagatttcactgccaactcctattat |
| | ggctggttgccgtactccaactcggcagtttagttcatgcgttgttattgaagctggtgattcattaaagtcaattaataaagcttctgcttcaattgttgaatatatttctaaacgcgctggaattgg |
| | aattaacgttggtatgattcgtgccgaaggttctaagattggcatgggtgaagtacgccatactggtgttattcctttttggaaacattttcagactgctgttaaatcatgttcacagggtggaattc |
| | gtggcggcgctgctactgcttattatcctatttggcatttggaagttgaaaatcttctcgttttgaaaaataacaaaggcgtagaagaaaaccgcatccgtcatatggattatggtgttcaactga |
| | atgatttgatgatggaacgattcggaaagaacgattacattactttgttcagtccgcatgaaatgggtggagagctgtattattcttattttaaagaccaagaccgtttccgtgaattatacgaagc |
| | agcagaaaaagaccctaatattcgtaaaaagcgtattaaagcccgtgaactatttgaattgctcatgactgaacgttcaggaacagcaaggatttatgtgcagttcattgataatacgaataact |
| | atactccgtttattcgtgaaaaggcacctattcgtcagagtaacttgtgctgtgaaattgctattccaacaaatgatgtgaatagtcctgatgctgaaattggattgtgtactctctctgcattcgtac |
| | tagataattttgactggcaagaccaagataaaattaatgaattggcagaagttcaagttcgtgctcttgataatctgttggattaccaaggatatccagttcctgaagcagaaaaagctaaaaag |
| | cgtcgtaaccttggtgtaggtgttactaactatgcagcttggctggcaagtaactttgcttcttatgaagatgctaacgatttaacacatgaactatttgagagattacagtatggactcattaaag |
| | catccattaagctcgccaaagaaaaaggaccttgtgaatattattcagacactcgttggtctcgaggcgaattacctatcgactggtacaataaaaagattgaccaaatcgcagctccaaaata |
| | cgtttgtgactggtcgtcgctgcgggaagaccttaagctctttggcatccgtaatagcacattatcagcacttatgccatgtgagtcatcttcccaagtttctaacagtacaaacggtatcgagc |
| | ctccacgtggaccagtctctgttaaagaatcaaaagagggttcctttaatcaagtcgtgcccaatattgaacataacatagacctatatgattatacatggaaattagctaagaaaggtaataaa |
| | ccttatcttacgcaggtagctattatgctgaaatgggtatgtcaatcagcttcagcgaatacatattatgacccgcagatttttccaaaaggaaaggttccaatgtcaataatgattgatgacatgt |
| | tatacggatggtattatggcattaaaaatttctattatcataatacccgcgatggttctggtactgatgattatgaaatagaaactccaaaagctgaagattgttcatcctgtaaattatga (SEQ |
| | ID NO: 463) |
| 53 | atgagattacaacgccaaagcatcaaagattcagaagttagaggtaaatggtattttaatatcatcggtaaagattctgaacttgttgaaaaagctgaacatcttttacgtgatatgggatggga |
| | agatgaatgcgatggatgtcctctttatgaagacggagaaagcgcaggattctggatttaccattctgacgtcgagcagtttaaaactgattggaaaattgtgaaaaagtctgtttgaaggaga |
| | tgatatgatttttgtatttgaatttatgaatgatgaattcgattatgcaatttttaacgcattgcataatcctgatttaaatgaatttaatgaaatgttttctgacgctttgagtatgtcagaagaatactgc |
| | ggagaatgtcaacgtgtttgtgtgacagtctttgaaaacaaagaaaagacgtatgaagaattattctttgacgctaataaagccactgaatggtttattgaaaggggttttgcgtaatgattaaatt |
| | ggtattcgcttattctccaactaaaacggtcgaaggctttaatgaattagcattcggtttatgtgatggtttaccatggggacgagttaaaaaggacctccagaattttaaagctcgtactgaagg |
| | tacaattatgattatgggtgctaaaacgttccagtcattgtctacattacttcctggtcgtagccatattgtagtatgtgacctcgagcgtgattatcctgaaactaaagacggtgatttagcacattt |
| | ctatattacatgggagcagtacataacttacatttctggcggttcaattcaagtgtcaagtcctaatgcaccattcgaggctatgcttgatcagaattctaatgtaagcgtaattggcggacccgc |
| | tttgttatatgctgcattaccttgtgcggatgaagtagttgtttctcgcatcgttaaaaggcatcgtgttaattcaacggttcaattagatgcaagttttcttgatgatataagcaagcgtgaaatggtt |
| | gaaacccattggtataaaatagatgaagtaacaacccttacggaatcagtgtataaatgaaataacgcgtggcggaaaatatgaactttaattattaccctattctattagaaaaagacgcgaa |
| | acaaccaaaatggcagggtcctcagtttattaaaggcgtctatcaattagtagttcctaaagacaagatttatagcagttgtttcactgaatccgcttgcagtattttcggtaatagttctccgtatt |
| | ggaattttgatataaaactggatagaaatatcgatatttggttgaaagccatggatattggcaatattacgtttgatgagaataattatcatattattggtcgcttttctaaacgcggtaaagaattat |
| | atttcactcctgaaatcgaaagaaaatttgatgctaaaccgtattga (SEQ ID NO: 464) |
| 54 | atgtatattggcaaaaagtatgaacttgttccaagacttattgatacatttattaattatcgcccacgttctaattcatcaatagttaaaattattgaagaaaatggcgggtggtttgaagttaaagaa |
| | actttctttgttgatggatttagagcaataaaacacattgaatgcgcaaatggaaagcatttttactttaacatttgtgaagatgaatttcattgttttcgtgagtataaagaacagacttctgaagaa |
| | gatgaaatcgaagacaaggtttctggcgtaacaaaaattcactgcattgtagacgaaaacaatgtagatgaaatcattgaacttttgcgaaaaactttcaaaaagtag (SEQ ID NO: |
| | 465) |
| 55 | atggctaaagttgatattgacatcgttgattttgaatatattgaagaaattattcgtaatcgttatcctgaacttagtatcacaagcgtgcaagattctaagttttggagtattcaaatcgttattgaag |
| | gtcctcttgaagacctcacccgctttatggctaatgaatattgcgatggtatggattctgaagacgcagaattttacatgggactgattgaacaataa (SEQ ID NO: 466) |
| 56 | atgtttaaacgtaaatctactgctgaactcgctgcacaaatggctaaactggctggaaataaaggtggtttttcttctgaagataaaggcgagtggaaactgaaactcgataatgcgggtaacg |
| | gtcaagcagtaattcgttttcttccgtctaaaaatgatgaacaagcaccatttgcaattcttgtaaatcacggtttcaagaaaaacggtaaatggtatatcgaaaattgctcatctacccacggtga |
| | ttacgattcttgtccagtatgtcagtacatcagtaaaaatgatttgtacaacactgacaataaagagtacggtcttgttaaacgtaaaacttcttactgggctaacattcttgtagtaaaagatccag |
| | ctgctccagaaaacgaaggtaaagtatttaaataccgtttcggtaagaaaatctgggataaaatcaatgcaatgattgcagttgatgttgaaatgggtgaaactccggttgatgtaacttgtccg |
| | tgggaaggtgctaactttgtactgaaagttaaacaagtttccggatttagtaactacgacgaatctaaattcctgaatcaatctgcgattccaaacattgacgatgaatctttccagaaagaactg |
| | ttcgaacaaatggttgacctttctgaaatgacttctaaagataaattcaaatcgttcgaagaactgagcactaagtttagtcaagttatgggaactgctgctatgggtggtgccgcagcgactgc |
| | tgctaagaaagctgataaagttgctgatgatttggatgcattcaatgttgatgacttcaatacaaaaactgaagatgattttatgagctcaagctctggcagttcatctagtgctgatgacacgga |
| | cctggatgaccttttgaatgacctttaa (SEQ ID NO: 467) |
| 57 | atggatttagaaatgatgctggatgaagattacaaagagggaatttgctttattgactttagtcaaattgcgctttcaactgctttagtaaacttcccagataaagaaaaaattaatttatcaatggtt |
| | cgtcatttgatattgaactcaattaagtttaatgtcaaaaaagcaaaaacgcttggatacactaaaatcgtgttgtgtattgataacgcgaaatctggatattggcgtcgtgattttgcttattattata |
| | agaaaaaccgtggaaaagcacgagaagaatctacttgggactgggaaggttattttgaatccagccataaagttatagatgaattgaaagcttatatgccatacattgttatggatattgataag |
| | tatgaagcggatgaccatattgctgttcttgttaaaaagttctctttagaaggacataagattttaatcatttcgtcggatggtgactttacacagcttcacaaatatccaaatgttaagcaatggtct |
| | ccaatgcataagaaatgggttaaaattaaaagcggttctgctgaaattgactgtatgactaaaatccttaaaggcgacaaaaaggataacgttgcttcagttaaagtacgatctgacttctggttt |
| | accagagttgaaggtgaacgaactccttcaatgaaaacttcaatcgttgaagccattgctaatgaccgtgagcaagctaaggtgcttctcacagaatctgaatataatcgttataaagaaaattt |
| | agttctaattgattttgattatattcctgataatattgcttcaaacattgtgaattactataattcatataaattaccaccgcgtggcaaaatttattcatattttgtaaaagcgggtctttctaaattaacta |
| | atagcattaatgaattttgaggtgaataatggctaaaaaagaaatggttgaatttgatgaagctatccatggcgaagacttggctaaatttattaaagaagcatctgatcataaactgaaaatttcc |
| | ggttataatgaactgattaaagatattcgaattcgtgctaaagatgaacttggcgttgatggtaagatgtttaatcgtctattagctttgtatcataaagataaccgtgatgtgtttgaagctgaaact |
| | gaagaggtagttgaactttatgacacagttttctctaaatgatattcgtccggtcgatgagaccggtctttcagaaaaagaactttcaatcaagaaagaaaaggatgaaatagcaaagcttcttg |
| | atcgtcaagaaaatggatttattattgaaaaaatggtagaagagtttggaatgagttatcttgaagctacaacagcattcttagaagaaaattctattcctgaaactcaatttgctaaatttattcctt |
| | cgggtataattgaaaaaattcagtcagaagctattgacgaaaatcttttacgtccttctgttgttcgctgtgaaaaaactaatacattagattttctactatgattaaattccgcatgcctgctggtgg |
| | tgaaagatacattgatggtaaatcagtttataaattatacttaatgataaaacagcatatgaatggaaagtatgatgttattaagtataattggtgcatgcgggtgtctgatgccgcttatcaaaag |
| | cgaagggataagtattttttccagaagttatcagaaaaatataaattaaaggaacttgctttaatttttataagtaatttggttgctaaccaagatgcttggattggtgacatctctgacgctgatgca |
| | cttgtgttttatcgtgaatatatcggacgcttaaagcaaattaaatttaagtttgaagaagatattcgcaacatttattattttagtaaaaaagttgaagtttctgcttttaaagaaatctttgaatataat |
| | ccaaaggttcaatcaagttatatttttaaactgcttcagtcgaatataatttcgtttgaaacgtttatcttgcttgattcgtttttaaatataattgataaacacgatgaacagactgataatttagtctgg |
| | aataattattctataaagttaaaggcttatagaaaaattttaaatattgattcacagaaagctaaaaatgttttcattgaaactgtgaaatcttgcaagtattaa (SEQ ID NO: 468) |
| 58 | atggccgagattaaaagaaagttcagagcagaagatggtctggacgcaggtggtgataaaataatcaacgtagctttagctgatcgtgccgtaggaactgacggtgttaacgttgattactta |
| | attcaagaaaatacagttcaacaatatgatccaactcgtggatatttaaaagattttgtaatcatttatgataaccgcttttgggctgctataaatgatattccaaaaccagcaggagcttttaatag |
| | cggacgctggagagcattacgtaccgatgcaaactggattacggtttcatccggttcatatcaattaaaatccggtgaagcaatttcggttaatactgcagctggaaatgacatcacgtttactt |
| | taccatcttctccaattgatggtgatactatcgttctccaagatattggaggaaaacccggagttaaccaagttttaattgtagctccagtgcaaagtattgtaaactttagaggtgaacaagtac |
| | gttcagtactaatgactcatccaaagtcacagctagttttaatttttagtaatcgtctgtggcaaatgtatgttgctgattatagtagagaagctgtaattgtaacaccagcgaatacttatcaagca |
| | caatcaaacgattttatcgtgcatagatttacttctgccgcaccgataaatattaaacttccgagatttgctaatcacggagatattattaatttcgttgatttagataaactaaatccactttatcatac |
| | aattgttactacatacgatgaaactacttcaatacaagaagatggaactcattctattgaagaccgtacatcaatcgacggtttcttgatgtttgatgataatgagaaattgtggagattgtttgacg |
| | gggacagtaaagcacgtttacgtatcataacgactaattcaaacattcttccaaatgaagaagttatggtatttggtgcgaataacggaacaactcaaacaattgagcttcagcttccaactaat |
| | atttctgttggtgatactgttaaaatttccatgaattacatgagaaaaggacaaacagttaaaatcaaagctgctgatgaagataaaattgcttcttcagttcaattactgcaattcccaaaacgctc |
| | agaatatccgcctgaagctgaatgggtaactgtccaagaattagtttttaacggtgaaactaattatgttccagttttggagcttgcttatattgaagattctgatggaaaatactgggttgtacag |
| | caaaacgttccaaccgtagaaagagtagattctttaaatgattctactagagcaagattaggcgtaattgattagctacacaagctcaagctaacgtcgatttagaaaattctccacaaaaaga |
| | attagcaattactccagaaacgttagctaatcgcactgctactgaaactcgcagaggtattgcaagaatagcaactactgctcaagtgaatcagaacaccacattctcttttgctgacgatattat |
| | catcactcctaaaaagctgaatgaaagaactgctactgaaactcgcagaggtgttgctgaaattgctacgcagcaagaaactaatacaggtactgatgatactacaatcatcactcctaaaaa |
| | gcttcaagcccgtcaaggttctgaatcattatctggtattgtaacttttgtatctactgcaggtgctactccagcttctagccgtgaattaaatggtacgaatgtttataataaaaacactaataattta |
| | gttgtttcacctaaagctttggatcagtataaagctactccaacgcagcaaggtgcagtaattttagcagttgaaagtgaagtaattgctggaaaaagtcaggaaggatgggcgaatgctgttg |
| | taacgccagaaacgttacataaaaagacatcaactgatggaagaattggtttaattgaaattgctacgcaaagtgaagttaatacaggaactgattatactcgtgcagtcactcctaaaacttta |
| | aatgaccgtagagcaactgaaagtttaagtggtatagctgaaattgctacacaagttgaattcgacgcaggcgtcgacgatactcgtatctctacaccattaaaaattaaaaccagatttaata |
| | gtactgatcgtacttctgttgttgctctatctggattaattgaatcaggaactctctgggaccattatacccttaatattcttgaagcaaatgagacacaacgtggtacacttcgtgtagctacacaa |
| | gttgaagctgctgcaggaaaattagataatgttttaataactcctaaaaagcttttaggtactaaatctaccgaatcgcaagagggtgttattaaagttgcaactcagtctgaagctgtggctgga |
| | acgtcagcaaatactgctatatctccaaaaaatttaaaatggattgtgcagagtgaaccttcttggagagcaactactacggtaagagggtttgttaaaacttcgtctggttcaattacattcgttg |
| | gtaatgatacagtcggttctacccaagatttagaactttatgagaaaaataattatgcagtatcaccatatgaattaaaccgtgtattagcaaattatttgccgttaaaagcaaaagctgtagatag |
| | taatttattggatggtctagattcatcccagttcattcgtagggatattgcacagacggttaatggttcactaaccttaacccaacaaacgaatctgagtgcccctcttgtatcatctagtactgcta |
| | cgtttggtggttcagtttcggcaaatagtacattaactatttctaatactggtacgacttcttctcgatttacatttgagaaaggtcctgcttctggtagtaatgctgattctgcattgtatgttcgtgtat |
| | ggggtaataagtacagcggcggttctgatgtaactcgtgcaacgattatagaattctctgatgctaccggctctcatttctattctcaaagagatacgtcaaataatgtgttgttcaacatttcagg |
| | tacgatgcaatcagtcaacgctagcgttcgtggtgttctgaacgttacaggtgtctcaacgtttaatagttcagttacagccaatggtgaattcatcagtaaatcaccaaatgcttttagagcaat |
| | aaatggaaattacggattctttattcgtaatgctggtaatgacacctattttatgctcactgcagcaggtgatcagagcggtggatttaatggattacgtccattatcaattaataatcaatccggtc |
| | aggttacgattggtgaaagcttaatcattgccaaaggtgctactataaattcaggtggtttgactgttaactcgagaattcgttctcagggtactaaaacatctgatttatatacccgtgcgccaac |
| | atctgatactgtaggattctggtcaatcgatattaatgattcagccacttataaccagttcccgggttattttaaaatggttgaaaaaactaatgaagtgactgggcttccatacttagaacgtggc |
| | gaagaagttaaatctcctggtacactgactcagtttggtaatacacttgattcgctttaccaagattggattacttatccaacgacgccagaagcgcgtaccactcgctggacacgtacatggc |
| | agaaaaccaaaaactcttggtcaagttttgttcaggtatttgatggtggaaaccctcctcaaccttctgatattggtgctttaccttctgataatgcaacaatcggaaacttgacaataagggattt |
| | cttaaggattggtaatgtccgcattattccagaccctgtgaataaatctgttaaattcgagtggattgaataagaggtattatggaaaaatttatggctaagtttggacaaggatacgtccaaacg |
| | ccatttttatcggaaagcaattcagtacgatttaaattaagcatagcgggatcttgcccgctttctacagcaggaccatacgttaaatttcaagataatcctgtaggaagtcaaacatttagcgca |
| | ggtcttcatttaagagtttttgacccttccaccggagcattagttgatagtaagtcatatgctttttcgacttcaaatgatactacatcagctgcttttgttagcttcatgaattctttgacaaataatag |
| | aattgttgctatattaactaacggaaaggttaattttcctcctgaagtagtatcttggttaagaactgcaggaacgtctgcttttccatctgattctatattgtcaagattcgacgtatcatatgctgctt |
| | tttatacttcttctaaaagagctattgcattagagcatgttaaactgagtaatagaaaaagcacagatgattatcaaactattttagatgtcgtatttgacagtttagaagatgttggagctaccggg |
| | tttccaagaagaacgtatgaaagcgttgagcaatttatgtcagcggttggtggaactaataacgaaatcgcgcgtttaccaacttcggctgctataagtaaattatctgattataatttaattcctg |
| | gtgatgttctttatcttaaaacacagctatacgccgatgccgatttacttgctcttggaactacgaatatatccattcgattttataatgcatcaaatggatatatttcctcgacacaagctgaatttac |
| | cgggcaagctggtgtttgggaattaaaagaagattatgtagttgttccagaaaatgcagtaggatttacgatatatgcacaaagaactgcacaagctggtcaaggtggaatgaggaacttaa |
| | gcttttctgaggtatcaagaaatggtagtatttcgaaacccgctgaatttggtgtcaatggtattcgagttaattatgtctgtgaatctgcttcacctccggatataatggtacttcctacacaagcat |
| | cgtctaaaactggtaaagtgtttgggcaagaatttagagaagtataa (SEQ ID NO: 469) |
| 59 | atgtttactacagctgaactaaaacgagcaaaagctaagaaagggcaaggaaaatataaagctgaattagttaaagaacttcagtttgctgaggctgaattgaattcaatgattattcaaaatg |
| | ctccagaaactgaaattgctcttaaacgtattgcgaataagtgtcttcgtgatgcaatcgtcgatcttttagcggattattgagtaaaatgaaaatcgttgagattgaactatgagttcattatggtg |
| | gtgttttgtttggttaattagtattccattaatttgtttaacatttacttttgtgatgaggttattatgaaaatttttaattctgtacttattgcttgtgcgtggtgggttgcacaagtttcggcagtagtgattg |
| | gtattcacatttattacgaatatttttaa (SEQ ID NO: 470) |
| 60 | atgtacaatattaaatgcctgaccaaaaacgaacaagctgaaattgttaaactgtattcaagtggtaattacacccaacaggaattggctgattggcaaggtgtatcggttgacacaatccgtc |
| | gtgttttgaaaaatgctgaagaagctaaacgccctaaagttactattagcggtgatattacagttaaagttaatagcgatgcagttattgctccagttgctaaatctgacattatttggaatgcatct |
| | aaaaaattcatttcaattactgttgacggtgtaacttataacgcaactcctaatactcattcaaactttcaggaaattcttaatctgcttgtagcggataagctggaagaagctgcgcaaaaaatta |
| | atgttcgtcgcgctgttgaaaaatatatttccggcgatgttcgaattgaaggtggaagcttgttctatcaaaatattgaattgcggtctggtttggttgatcgtattcttgactcgacggaaaaagg |
| | cgaaaactttgaattttattttccgttcttggaaaatctgctggaaaacccaagccaaaaagcggtatctcgactctttgatttcttggtagcaaacgatattgaaatcaccgaagatggttacttct |
| | atgcttggaaagtagttcgtgacaactactttgactgtcactcaaacacctttgataacagtccgggtaaagtagttaaaatgccacgtactcgtgtgaatgacgatgatacacaaacttgttctc |
| | gtggtctgcatgtgtgttctaaatcttatattcgtcactttggcagttcaaccagtcgagttgtaaaagttaaagtacatccgcgtgatgtagtatcaattccgattgattacaacgatgctaaaatg |
| | cgtacctgccaatacgaagtagttgaagacgttactgaacaatttaaataagggcttcggcccttatcatattaaggaaaattatgttaggttatcaagcacgagtaaaagaagaatacgatca |
| | attaatgctcaaaattaatgcactgagtaaatttttagaaagcacaaagtttctaacggttagtgcagttgagcaagaactgctactttcgcagtttatctcaatgaaatcttatgctgagtgtctag |
| | agaaaagaattgcgcaattcaaataa (SEQ ID NO: 471) |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. An engineered system comprising an ATPase and an adenosine deaminase wherein the ATPase and the adenosine deaminase are derived from same or different prokaryotes.

2. The engineered system of claim 1, wherein the ATPase comprises a sequence of WP_012906049.1 or WP_155731552.1, and the adenosine deaminase comprises a sequence of WP_012906048.1 or WP_064360593.1.

3. The engineered system of claim 1, wherein the ATPase comprises 1100 or less amino acid residues.

4. The engineered system of claim 1, wherein the adenosine deaminase comprises 1100 or less amino acid residues.

5. The engineered system of claim 1, further comprising a membrane protein.

6. The engineered system of claim 5, wherein the membrane protein comprises a SLATT domain or Csx27.

7. The engineered system of claim 1, wherein the system is configured to modify a target nucleic acid.

8. The engineered system of claim 7, wherein the target nucleic acid is RNA.

9. The engineered system of claim 7, wherein modification of the target nucleic acid comprises causing an A to G mutation in the target nucleic acid.

10. The engineered system of claim 1, further comprising one or more phage proteins.

11. The engineered system of claim 10, wherein the one or more phage proteins are in Tables 18A-18B.

12. An engineered system comprising one or more reverse transcriptases comprising one or more UG1, UG2, UG3, UG8, UG15, or UG16 reverse transcriptase.

13. The engineered system of claim 12, comprising a first and a second reverse transcriptase.

14. The engineered system of claim 13, wherein the first and the second reverse transcriptases are comprised in a protein.

15. The engineered system of claim 12, further comprising:
a SLATT domain;
a DNA polymerase;
a family A DNA polymerase;
a serine protease domain linked to or associated with the one or more reverse transcriptases;
an MBL domain;
a nitrilase;
a nitrilase, wherein the nitrilase and the one or more reverse transcriptases are comprised in a protein, and the nitrilase is at a C-terminus of the protein; or
a protease.

16. (canceled)

17. (canceled)

18. (canceled)

19. (canceled)

20. (canceled)

21. (canceled)

22. (canceled)

23. The engineered system of claim 12, wherein the one or more reverse transcriptase comprises (Y/F)XDD (SEQ ID NOS: 1-2), wherein X is any amino acid.

24. An engineered system comprising a retron or one or more molecules encoded by the retron.

25. The engineered system of claim 24, wherein the retron is an Ec67 retron, Ec86 retron, or Ec78 retron.

26. (canceled)

27. (canceled)

28. The engineered system of claim 24, wherein the retron is a Tol/interleukin 1 (TIR) domain-associated retron.

29. The engineered system of claim 28, wherein the TIR domain has NAD+ hydrolase activity.

30. The engineered system of claim 24, wherein the retron is a topoisomerase-primase (TOPRIM) domain-associated retron.

31. The engineered system of claim 30, wherein the TOPRIM domain has nuclease activity.

32. An engineered system comprising:
an NTPase of a STAND (signal transduction ATPases with numerous associated domains) superfamily;
an NTPase of a STAND superfamily, DUF4297, Mrr-like nuclease, SIR2, a trypsin-like serine protease, and/or a helical domain;
von Willebrand factor (VWF), a PP2C-like serine/threonine protein phosphatase, and a serine/threonine kinase;
SIR2;
transmembrane ATPase;
ATPase, QueC synthase n, and TatD endonuclease;
S8 peptidase;
DUF4011, a helicase, and a Vsr endonuclease;
a silent information regulator (SIR)2-DUF4020;
SIR2-STAND-TPR;
a Polymerase and Histidinol Phosphatase (PHP)-ATPase;
SIR2 and HerA;
DUF1887;
DUF499, DUF3780, and DUF1156 methyltransferase and a helicase;
a Type I-E CRISPR-associated ATPase; or
ApeA.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. The system of claim 1, wherein the system comprises two proteins fused together.

50. The system of claim 1, comprising one or more components in a retrotransposon system.

51. A polynucleotide comprising coding sequences for one or more proteins in the system of claim 1.

52. A vector comprising a polynucleotide of claim 51.

53. A cell comprising the polynucleotide of claim 51.

54. A method of identifying a defense system in a microorganism, the method comprising:
identifying genes of known defense systems in a plurality of genomes of the microorganism;
recording candidate genes located within 10 kb or 10 open reading frames from the identified genes of known defense systems in the genomes;
identifying homologs of each candidate gene in the genomes; and
selecting candidate genes wherein at least 10% of homologs of the candidate genes are within 5000 nucleotides or 5 genes from one or more known defense systems on the genomes.

55. The method of claim 54, wherein identifying genes of known defense systems comprises identifying known defense genes and filtering false positive hits among the identified known defense genes.

56. The method of claim 54, further comprising validating the selected candidate genes.

57. The method of claim 54, wherein the homologs of the candidate genes share at least 70% sequence identity with the candidate genes and/or the homologs have an E-value of 10⁻⁵ or lower.

58. The method of claim 54, wherein the recorded candidate genes are within 10 kb from the identified genes of known defense systems on the genomes.

59. The method of claim 54, wherein at least 15% of homologs of the selected candidate genes are within 5000 nucleotides or 5 genes from one or more known defense systems on the genomes.

60. The method of claim 54, wherein the plurality of genomes comprises at least 100,000 genomes.

61. The method of claim 54, wherein the known defense systems comprise one or more of a CRISPR system, Type I RM and McrBC system, BREX-associated system, Zorya system, Wadjet system, Druantia-associated system, Hachiman system, Lamassu system, Thoeris-like system, Gabija system, Septu system, pAgo system, Shedu system, Kiwa system, DUF499-DUF1156 system, and Toxin/antitoxin system.

62. The method of claim 54, wherein the microorganism is E. coli.
